(19) United States
(12) Patent Application Publication
(10) Pub. No.: US 2021/0074926 A1
(43) Pub. Date: Mar. 11, 2021

(54) Compound For Organic Electronic Element, Organic Electronic Element Using Same, And Electronic Device Thereof

(71) Applicant: Duk San Neolux Co., Ltd, Cheonan-si (KR)

(72) Inventors: Jeong Keun PARK, Seoul (KR); Hye Ryeong KIM, Cheonan-si (KR); Ho Young JUNG, Cheonan-si (KR); Jung Hwan PARK, Hwaseong-si (KR); Sun Hee LEE, Cheonan-si (KR); Gyu Min LEE, Cheonan-si (KR); Sun Pil HWANG, Ansan-si (KR); Seok Hyun KIM, Seongnam-si (KR)

(21) Appl. No.: 16/950,339

(22) Filed: Nov. 17, 2020

Related U.S. Application Data
(63) Continuation of application No. 16/794,653, filed on Feb. 19, 2020.
Application No. 16/794,653 is a continuation of application No. 15/317,797, filed on Dec. 9, 2016.

(30) Foreign Application Priority Data
Jun. 12, 2014 (KR) 10-2014-0071264
Jun. 20, 2014 (KR) 10-2014-0076034
Jul. 7, 2014 (KR) 10-2014-0084320
Aug. 8, 2014 (KR) 10-2014-0102197

(51) Int. Cl. H01L 51/00; C07D 471/04; C09K 11/06; C07D 403/04; C07D 209/82; C07D 403/14; C09K 11/02

(52) U.S. Cl. CPC H01L 51/0072; C07D 471/04; C09K 11/06; C07D 403/04; C07D 209/82; H01L 51/506; C09K 11/02; H01L 51/0052; H01L 51/0061; H01L 51/0067; C07D 403/14

(57) ABSTRACT

The present invention provides a compound that can increase the light-emitting efficiency, reduce the driving voltage, and improve the durability of an element, an organic electronic element using the same, and an electronic device thereof.

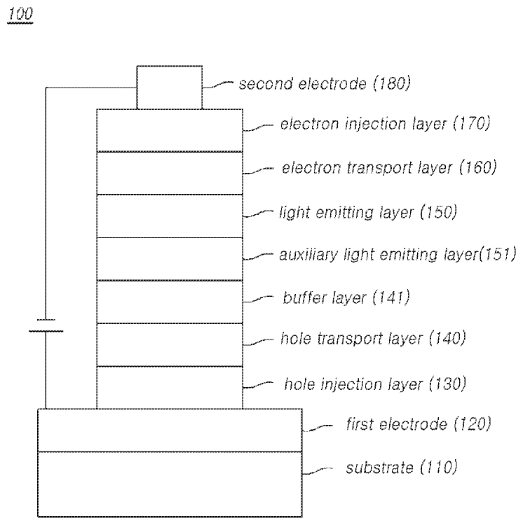

### CROSS-REFERENCE TO RELATED APPLICATIONS

This application is a continuation of U.S. patent application Ser. No. 16/794,653 filed Feb. 19, 2020, which is a continuation of U.S. Pat. No. 15,317,797 filed Dec. 9, 2016, which is a U.S. National Stage application of International Application No. PCT/KR2015/005938 filed on Jun. 12, 2015, which claims priority to Korean Patent Application No. 10-2014-0102197 filed Aug. 8, 2014, Korean Patent Application No. 10-2014-0071264 filed Jun. 12, 2014, Korean Patent Application No. 10-2014-0076034 filed Jun. 20, 2014, and Korean Patent Application No. 10-2014-0084320 filed Jul. 7, 2014, all of which are hereby expressly incorporated by reference herein in their entireties.

### TECHNICAL FIELD

The present invention relates to a compound for an organic electronic element, an organic electronic element using the same, and an electronic device thereof.

### BACKGROUND ART

In general, an organic light emitting phenomenon refers to a phenomenon in which electric energy is converted into light energy of an organic material using an organic material. An organic electronic element utilizing the organic light emitting phenomenon usually has a structure including an anode, a cathode, and an organic material layer interposed therebetween. In many cases, the organic material layer may have a multilayered structure including multiple layers made of different materials in order to improve the efficiency and stability of an organic electronic element, and for example, may include a hole injection layer, a hole transport layer, a light emitting layer, an electron transport layer, an electron injection layer, or the like.

A material used as an organic material layer in an organic electronic element may be classified into a light emitting material and a charge transport material, for example, a hole injection material, a hole transport material, an electron transport material, an electron injection material, and the like according to its function.

Further, the light emitting material may be divided into a high molecular weight type and a low molecular weight type according to its molecular weight, and may also be divided into a fluorescent material derived from electronic excited singlet states and a phosphorescent material derived from electronic excited triplet states according to its light emitting mechanism. Further, the light emitting material may be divided into blue, green, and red light emitting materials and yellow and orange light emitting materials required for better natural color reproduction according to its light emitting color.

When only one material is used as a light emitting material, there occur problems of shift of a maximum luminescence wavelength to a longer wavelength due to intermolecular interactions and lowering of the efficiency of a corresponding element due to the deterioration in color purity or a reduction in luminous efficiency. On account of this, a host/dopant system may be used as the light emitting material in order to enhance the color purity and increase the luminous efficiency through energy transfer. This is based on the principle that if a small amount of dopant having a smaller energy band gap than a host forming a light emitting layer is mixed in the light emitting layer, then excitons generated in the light emitting layer are transported to the dopant, thus emitting light with high efficiency. With regard to this, since the wavelength of the host is shifted to the wavelength band of the dopant, light having a desired wavelength can be obtained according the type of the dopant.

Currently, the power consumption is required more and more as the size of display becomes larger and larger in the portable display market. Therefore, the power consumption is a very important factor in the portable display with a limited power source of the battery, and efficiency and life span issue also is solved.

Efficiency, life span, driving voltage, and the like are correlated with each other. For example, if efficiency is increased, then driving voltage is relatively lowered, and the crystallization of an organic material due to Joule heating generated during operation is reduced as driving voltage is lowered, as a result of which life span shows a tendency to increase. However, efficiency cannot be maximized only by simply improving the organic material layer. This is because long life span and high efficiency can be simultaneously achieved when an optimal combination of energy levels and T1 values, inherent material properties (mobility, interfacial properties, etc.), and the like among the respective layers included in the organic material layer is given.

That is, in order to allow the organic electronic element to sufficiently exhibit excellent characteristics, most of all, materials constituting an organic material layer in the element, for examples, a hole injection material, a hole transport material, a light emitting material, an electron transport material, an electron injection material, and the like need to be supported by stable and efficient materials, but the development of stable and efficient materials for the organic material layer for an organic electronic element is not sufficiently achieved. Therefore, the development of new materials is continuously required, and especially, the development of an electron transport material and a light emitting material is urgently required.

### DETAILED DESCRIPTION OF THE INVENTION

### Technical Problem

In order to solve the above-mentioned problems occurring in the prior art, an object of the present invention is to provide a compound capable of achieving high luminous efficiency, a low driving voltage, and an improved lifespan of an element, an organic electronic element using the same, and an electronic device.

### Technical Solution

In accordance with an aspect of the present invention, there is provided a compound represented by the following formula.

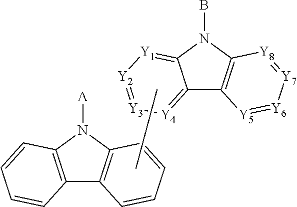

In accordance with another aspect of the present invention, there is provided a compound represented by the following formula.

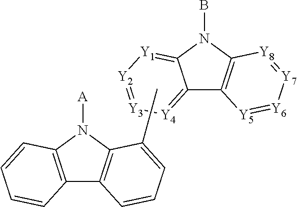

In accordance with another aspect of the present invention, there is provided a compound represented by the following formula.

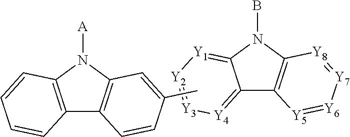

In accordance with another aspect of the present invention, there is provided a compound represented by the following formula.

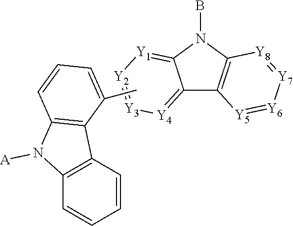

In accordance with another aspect of the present invention, there is provided a compound represented by the following formula.

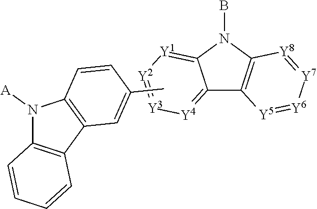

In another aspect of the present invention, there are provided an organic electronic element using the compound represented by the above formula, and an electronic device.

### Advantageous Effects

The use of the compound according to the present invention can achieve high luminous efficiency and a low driving voltage of an element and significantly improving an improved lifespan of an element.

### BRIEF DESCRIPTION OF THE DRAWING

The FIGURE illustrates an example of an organic electronic light emitting element according to the present invention.

### MODE FOR CARRYING OUT THE INVENTION

Hereinafter, some embodiments of the present invention will be described in detail with reference to the accompanying illustrative drawings

In designation of reference numerals to components in respective drawings, it should be noted that the same elements would be designated by the same reference numerals although they are shown in different drawings. Further, in the following description of the present invention, a detailed description of known functions and configurations incorporated herein will be omitted when it may make the subject matter of the present invention rather unclear.

In addition, terms, such as first, second, A, B, (a), (b), or the like may be used herein when describing components of the present invention. Each of these terminologies is not used to define an essence, order, or sequence of a corresponding component but used merely to distinguish the corresponding component from other component(s). It should be noted that if it is described in the specification that one component is “connected,” “coupled” or “joined” to another component, a third component may be “connected,” “coupled,” and “joined” between the first and second components, although the first component may be directly connected, coupled or joined to the second component.

As used in the specification and the accompanying claims, unless otherwise stated, the following is the meaning of the term as follows.

Unless otherwise stated, the term “halo” or “halogen” as used herein includes fluorine (F), bromine (Br), chlorine (Cl), and iodine (I).

Unless otherwise stated, the term “alkyl” or “alkyl group” as used herein has a single bond of 1 to 60 carbon atoms, and means aliphatic functional radicals including a linear alkyl group, a branched chain alkyl group, a cycloalkyl group (alicyclic), or an alkyl group substituted with a cycloalkyl.

Unless otherwise stated, the term “haloalkyl group” or “halogen alkyl group” as used herein means an alkyl group substituted with halogen.

The term “heteroalkyl group” as used herein means an alkyl group of which at least one of carbon atoms is substituted with a hetero atom.

Unless otherwise stated, the term “alkenyl” or “alkynyl” as used herein has, but not limited to, double or triple bonds of 2 to 60 carbon atoms, and includes a linear alkyl group, or a branched chain alkyl group.

Unless otherwise stated, the term “cycloalkyl” as used herein means, but not limited to, alkyl forming a ring having 3 to 60 carbon atoms.

The term “alkoxyl group”, “alkoxy group” or “alkyloxy group” as used herein means an alkyl group to which oxygen radical is attached, but not limited to, and, unless otherwise stated, has 1 to 60 carbon atoms.

The term “alkenoxyl group”, “alkenoxy group”, “alkenyloxyl group”, or “alkenyloxy group” as used herein means an alkenyl group to which oxygen radical is attached, but not limited to, and, unless otherwise stated, has 2 to 60 carbon atoms.

The term “aryloxyl group” or “aryloxy group” as used herein means an aryl group to which oxygen radical is attached to, but not limited to, and has 6 to 60 carbon atoms.

Unless otherwise stated, the terms “aryl group” and “arylene group” each have 6 to 60 carbon atoms, but not limited thereto. The aryl group or arylene group herein means a monocyclic or polycyclic aromatic group, and includes an aromatic ring that is formed in conjunction with an adjacent substituent linked thereto or participating in the reaction. Examples of the aryl group may include a phenyl group, a biphenylyl group, a terphenylyl group, a naphthyl group, an anthracenyl group, a fluorene group, a spirofluorene group, and a spirobifluorene group.

The prefix “aryl” or “ar” means a radical substituted with an aryl group. For example, an arylalkyl group may be an alkyl group substituted with an aryl group, and an arylalkenyl group may be an alkenyl group substituted with an aryl group, and a radical substituted with an aryl group has a number of carbon atoms defined as herein.

Also, when prefixes are named subsequently, it means that substituents are listed in the order described first.

For example, an arylalkoxy group means an alkoxy group substituted with an aryl group, an alkoxylcarbonyl group means a carbonyl group substituted with an alkoxyl group, and an arylcarbonylalkenyl group also means an alkenyl group substituted with an arylcarbonyl group, wherein the arylcarbonyl group may be a carbonyl group substituted with an aryl group.

Unless otherwise stated, the term “heteroalkyl” as used herein means alkyl containing one or more heteroatoms. Unless otherwise stated, the term “heteroaryl group” or “heteroarylene group” as used herein means, but not limited to, an aryl or arylene group having 2 to 60 carbon atoms and containing one or more heteroatoms, includes at least one of monocyclic and polycyclic rings, and may also be formed in conjunction with an adjacent group.

Unless otherwise stated, the term “heterocyclic group” as used herein contains one or more heteroatoms, has 2 to 60 carbon atoms, includes at least one of homocyclic and heterocyclic rings, and may also be formed in conjunction with an adjacent group.

Unless otherwise stated, the term “heteroatom” as used herein represents N, O, S, P, or Si.

In addition, the “heterocyclic group” also may include a ring containing SO2 instead of carbon forming the ring. For examples, the “heterocyclic group” includes the following compound.

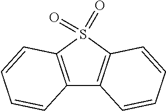 [SMILES: O=S1(=O)c2ccccc2-c2ccccc21]

Unless otherwise stated, the term “aliphatic” as used herein means an aliphatic hydrocarbon having 1 to 60 carbon atoms, and the term “aliphatic ring” as used herein means an aliphatic hydrocarbon ring having 3 to 60 carbon atoms.

Unless otherwise stated, the term “ring” means an aliphatic ring having 3 to 60 carbon atoms, an aromatic ring having 6 to 60 carbon atoms, a hetero ring having 2 to 60 carbon atoms, or a fused ring formed by the combination of them, and includes a saturated or unsaturated ring.

Hetero compounds or hetero radicals other than the above-mentioned hetero compounds each contain, but not limited to, one or more heteroatoms.

Unless otherwise stated, the term “carbonyl” as used herein is represented by —COR′, wherein R′ may be hydrogen, an alkyl having 1 to 20 carbon atoms, an aryl having 6 to 30 carbon atoms, a cycloalkyl having 3 to 30 carbon atoms, an alkenyl having 2 to 20 carbon atoms, an alkynyl having 2 to 20 carbon atoms, or the combination of these.

Unless otherwise stated, the term “ether” as used herein is represented by —R—O—R′, wherein R′ may be hydrogen, an alkyl having 1 to 20 carbon atoms, an aryl having 6 to 30 carbon atoms, a cycloalkyl having 3 to 30 carbon atoms, an alkenyl having 2 to 20 carbon atoms, an alkynyl having 2 to 20 carbon atoms, or the combination of these.

Unless otherwise stated, the term “substituted or unsubstituted” as used herein means that substitution is carried out by at least one substituent selected from the group consisting of, but not limited to, deuterium, halogen, an amino group, a nitrile group, a nitro group, a C₁-C₂₀ alkyl group, a C₁-C₂₀ alkoxyl group, a C₁-C₂₀ alkylamine group, a C₁-C₂₀ alkylthio group, a C₆-C₂₀ arylthio group, a C₂-C₂₀ alkenyl group, a C₂-C₂₀ alkynyl group, a C₃-C₂₀ cycloalkyl group, a C₆-C₆₀ aryl group, a C₆-C₂₀ aryl group substituted by deuterium, a C₈-C₂₀ arylalkenyl group, a silane group, a boron group, a germanium group, and a C₅-C₂₀ heterocyclic group.

Otherwise specified, the formulas used in the present invention are defined as in the index definition of the substituent of the following Formula.

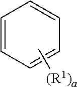

Wherein, when a is an integer of zero, the substituent R¹ is absent, when a is an integer of 1, the sole R¹ is linked to any one of the carbon atoms constituting the benzene ring, when a is an integer of 2 or 3, the substituent R¹'s may be the same and different, and are linked to the benzene ring as follows. When a is an integer of 4 to 6, the substituents R¹'s may be the same and different, and are linked to the benzene ring in a similar manner to that when a is an integer of 2 or 3, hydrogen atoms linked to carbon constituents of the benzene ring being not represented as usual.

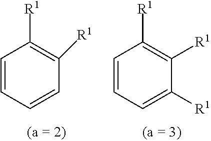

The FIGURE illustrates an organic electronic element according to an embodiment of the present invention.

Referring to the FIGURE, an organic electronic element 100 according to an embodiment of the present invention includes a first electrode 120 formed on a substrate 110, a second electrode 180, and an organic material layer between the first electrode 120 and the second electrode 180, which contains the compound of the present invention. Here, the first electrode 120 may be an anode (positive electrode), and the second electrode 180 may be a cathode (negative electrode). In the case of an inverted organic electronic element, the first electrode may be a cathode, and the second electrode may be an anode.

The organic material layer includes a hole injection layer 130, a hole transport layer 140, a light emitting layer 150, an electron transport layer 160, and an electron injection layer 170 formed in sequence on the first electrode 120. Here, the layers included in the organic material layer, except the light emitting layer 150, may not be formed. The organic material layer may further include a hole blocking layer, an electron blocking layer, an auxiliary light emitting layer 151, a buffer layer 141, etc., and the electron transport layer 160 and the like may serve as the hole blocking layer.

Although not shown, the organic electronic element according to an embodiment of the present invention may further include a protective layer or a light efficiency improving layer (capping layer) formed on at least one of the sides the first and second electrodes, which is a side opposite to the organic material layer.

The compound of the present invention employed in the organic material layer may be used as a host material, a dopant material, or a light efficiency layer material in the hole injection layer 130, the hole transport layer 140, the electron transport layer 160, the electron injection layer 170, or the light emitting layer 150. Preferably, the compound of the present invention may be used for the light emitting layer 150.

Since depending on the type and position of a substituent to be attached, a band gap, electrical properties, interfacial properties, and the like may vary even in the same core, it is very important what the types of core and a combination of substituent attached to the core are. Specially, long life span and high efficiency can be simultaneously achieved when an optimal combination of energy levels and T1 values, inherent material properties (mobility, interfacial properties, etc.), and the like among the respective layers included in the organic material layer is given.

Accordingly, in the present invention, a combination of energy levels and T1 values, inherent material properties (mobility, interfacial properties, etc.), and the like among the respective layers included in the organic material layer is optimized by forming a light emitting layer by using the compounds represented by Formulas 1-1 to 4-1, and thus the life span and efficiency of the organic electronic element can be improved at the same time.

The organic electronic element according to an embodiment of the present invention may be manufactured using a PVD (physical vapor deposition) method. For example, the organic electronic element may be manufactured by depositing a metal, a conductive metal oxide, or a mixture thereof on the substrate to form the anode 120, forming the organic material layer including the hole injection layer 130, the hole transport layer 140, the light emitting layer 150, the electron transport layer 160, and the electron injection layer 170 thereon, and then depositing a material, which can be used as the cathode 180, thereon.

Also, the organic material layer may be manufactured in such a manner that a smaller number of layers are formed using various polymer materials by a soluble process or solvent process, for example, spin coating, dip coating, doctor blading, screen printing, inkjet printing, or thermal transfer, instead of deposition. Since the organic material layer according to the present invention may be formed in various ways, the scope of protection of the present invention is not limited by a method of forming the organic material layer.

According to used materials, the organic electronic element according to an embodiment of the present invention may be of a top emission type, a bottom emission type, or a dual emission type.

A WOLED (White Organic Light Emitting Device) readily allows for the formation of ultra-high definition images, and is of excellent processability as well as enjoying the advantage of being produced using conventional color filter technologies for LCDs. In this regard, various structures for WOLEDs, used as back light units, have been, in the most part, suggested and patented. Representative among the structures are a parallel side-by-side arrangement of R (Red), G (Green), B (Blue) light-emitting units, a vertical stack arrangement of RGB light-emitting units, and a color conversion material (CCM) structure in which electroluminescence from a blue (B) organic light emitting layer, and photoluminescence from an inorganic luminescent using the electroluminescence are combined. The present invention is applicable to these WOLEDs.

Further, the organic electronic element according to an embodiment of the present invention may be any one of an organic light emitting diode (OLED), an organic solar cell, an organic photo conductor (OPC), an organic transistor (organic TFT), and an element for monochromatic or white illumination.

Another embodiment of the present invention provides an electronic device including a display device, which includes the above described organic electronic element, and a control unit for controlling the display device. Here, the electronic device may be a wired/wireless communication terminal which is currently used or will be used in the future, and covers all kinds of electronic devices including a mobile communication terminal such as a cellular phone, a personal digital assistant (PDA), an electronic dictionary, a point-to-multipoint (PMP), a remote controller, a navigation unit, a game player, various kinds of TVs, and various kinds of computers.

Hereinafter, a compound according to an aspect of the present invention will be described.

The compound according to an aspect of the present invention is represented by Formula 1 below.

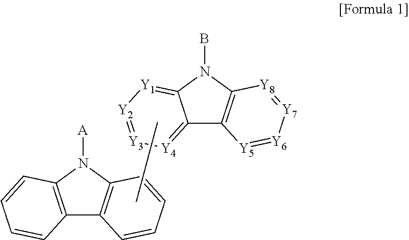

In Formula 1,

A and B each may be independently selected from the group consisting of a C₆-C₆₀ aryl group, a fluorenyl group, a C₂-C₆₀ heterocyclic group containing at least one heteroatom of O, N, S, Si, and P, a fused ring group of a C₃-C₆₀ aliphatic group and a C₆-C₆₀ aromatic group, a C₁-C₅₀ alkyl group, a C₂-C₂₀ alkenyl group, a C₂-C₂₀ alkynyl group, C₁-C₃₀ alkoxyl group, a C₆-C₃₀ aryloxy group, and -L′-N(Rₐ) (R_{b}).

L′ may be selected from the group consisting of a single bond, a C₆-C₆₀ arylene group, a fluorenyl group, a fused ring group of a C₃-C₆o aliphatic group and a C₆-C₆₀ aromatic group, and a C₂-C₆₀ heterocyclic group.

Rₐ and R_{b} each may be independently selected from the group consisting of a C₆-C₆₀ aryl group, a fluorenyl group, a fused ring group of a C₃-C₆₀ aliphatic group and a C₆-C₆₀ aromatic group, and a C₂-C₆₀ heterocyclic group containing at least one heteroatom of O, N, S, Si, and P.

Y₁ to Y₈ each may be independently CR or N, and at least one of Y₁ to Y₈ may be N.

At least one of R's may be linked to adjacent carbazole, and R that is not linked thereto may be hydrogen.

For example, when A, B, L′, Rₐ, and R_{b} are an aryl group, A, B, L′, Rₐ, and R_{b} each may be independently a phenyl group, a biphenyl group, a naphthyl group, or the like.

the aryl group, fluorenyl group, heterocyclic group, fused ring group, alkyl group, alkenyl group, alkoxyl group, aryloxy group, arylene group, and fluorenylene group each may be substituted with at least one substituent selected from the group consisting of deuterium, halogen, a silane group, a siloxane group, a boron group, a germanium group, a cyano group, a nitro group, a C₁-C₂₀ alkylthio group, a C₁-C₂₀ alkoxyl group, a C₁-C₂₀ alkyl group, a C₂-C₂₀ alkenyl group, a C₂-C₂₀ alkynyl group, a C₆-C₂₀ aryl group, a C₆-C₂₀ aryl group substituted with deuterium, a fluorenyl group, a C₂-C₂₀ heterocyclic group, a C₃-C₂₀ cycloalkyl group, a C₇-C₂₀ arylalkyl group, and a C₈-C₂₀ arylalkenyl group.

Here, the aryl group may be an aryl group having 6-60 carbon atoms, preferably 6-40 carbon atoms, and more preferably 6-30 carbon atoms;

the heterocyclic group may be a heterocyclic group having 2-60 carbon atoms, preferably 2-30 carbon atoms, and more preferably 2-20 carbon atoms;

the arylene group may be an arylene group having 6-60 carbon atoms, preferably 6-30 carbon atoms, and more preferably 6-20 carbon atoms; and

the alkyl group may be an alkyl group having 1-50 carbon atoms, preferably 1-30 carbon atoms, more preferably 1-20 carbon atoms, and especially preferably 1-10 carbon atoms.

Depending on the location of the carbazole at the left side in Formula 1, the present invention may be classified into <Example 1> indicated by Formula 1-1, <Example 2> indicated by Formula 2-1, <Example 3> indicated by Formula 3-1, and <Example 4> indicated by Formula 4-1. Hereinafter, the compounds in <Example 1> to <Example 4>, and synthesis examples, comparative examples, and element data thereof are described, but the present invention is not limited thereto.

### Example 1

The compound according to an aspect of the present invention is represented by Formula 1-1 below.

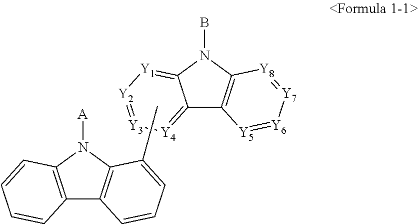

In Formula 1-1,

A and B each may be independently selected from the group consisting of a C₆-C₆₀ aryl group, a fluorenyl group, a C₂-C₆₀ heterocyclic group containing at least one heteroatom of O, N, S, Si, and P, a fused ring group of a C₃-C₆₀ aliphatic group and a C₆-C₆₀ aromatic group, a C₁-C₅₀ alkyl group, a C₂-C₂₀ alkenyl group, a C₂-C₂₀ alkynyl group, C₁-C₃₀ alkoxyl group, a C₆-C₃₀ aryloxy group, and -L′-N(Rₐ) (RID). L′ may be selected from the group consisting of a single bond, a C₆-C₆₀ arylene group, a fluorenyl group, a fused ring group of a C₃-C₆o aliphatic group and a C₆-C₆₀ aromatic group, and a C₂-C₆₀ heterocyclic group.

Rₐ and R_{b} each may be independently selected from the group consisting of a C₆-C₆₀ aryl group, a fluorenyl group, a fused ring group of a C₃-C₆₀ aliphatic group and a C₆-C₆o aromatic group, and a C₂-C₆₀ heterocyclic group containing at least one heteroatom of O, N, S, Si, and P.

Y₁ to Ye each may be independently CR or N, and at least one of Y₁ to Ye may be N.

At least one of R's may be linked to adjacent carbazole, and R that is not linked thereto may be hydrogen.

For example, when A, B, L′, Rₐ, and R_{b} are an aryl group, A, B, L′, Rₐ, and R_{b} each may be independently a phenyl group, a biphenyl group, a naphthyl group, or the like.

the aryl group, fluorenyl group, heterocyclic group, fused ring group, alkyl group, alkenyl group, alkoxyl group, aryloxy group, arylene group, and fluorenylene group each may be substituted with at least one substituent selected from the group consisting of deuterium, halogen, a silane group, a siloxane group, a boron group, a germanium group, a cyano group, a nitro group, a C₁-C₂₀ alkylthio group, a C₁-C₂₀ alkoxyl group, a C₁-C₂₀ alkyl group, a C₂-C₂₀ alkenyl group, a C₂-C₂₀ alkynyl group, a C₆-C₂₀ aryl group, a C₆-C₂₀ aryl group substituted with deuterium, a fluorenyl group, a C₂-C₂₀ heterocyclic group, a C₃-C₂₀ cycloalkyl group, a C₇-C₂₀ arylalkyl group, and a C₈-C₂₀ arylalkenyl group.

Here, the aryl group may be an aryl group having 6-60 carbon atoms, preferably 6-40 carbon atoms, and more preferably 6-30 carbon atoms;

the heterocyclic group may be a heterocyclic group having 2-60 carbon atoms, preferably 2-30 carbon atoms, and more preferably 2-20 carbon atoms;

the arylene group may be an arylene group having 6-60 carbon atoms, preferably 6-30 carbon atoms, and more preferably 6-20 carbon atoms; and

the alkyl group may be an alkyl group having 1-50 carbon atoms, preferably 1-30 carbon atoms, more preferably 1-20 carbon atoms, and especially preferably 1-10 carbon atoms.

Specifically, the compound represented by Formula 1-1 above may be expressed by one of the following compounds.

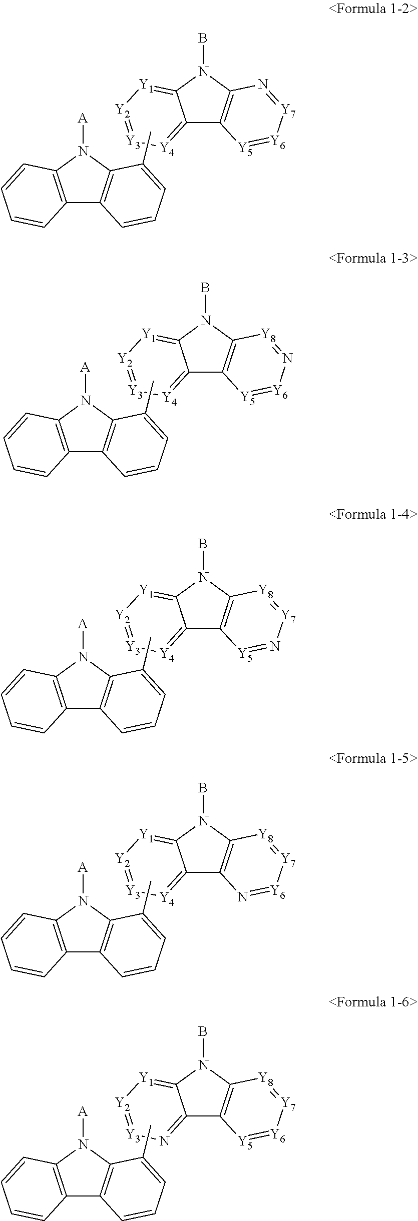

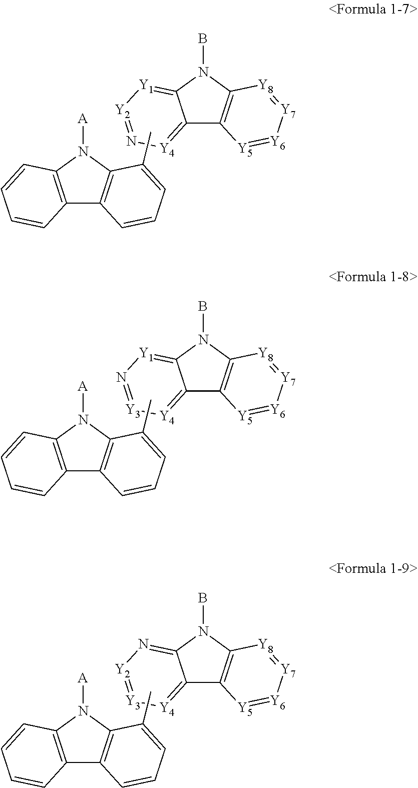

In Formulas 1-2 to 1-9,

Y₁ to Y₈ and A and B may be identical Y₁ to Y₈ and A and B defined in Formula 1-1.

More specifically, the compounds represented by Formulas 1-1 to 1-9 may be one of the following compounds.

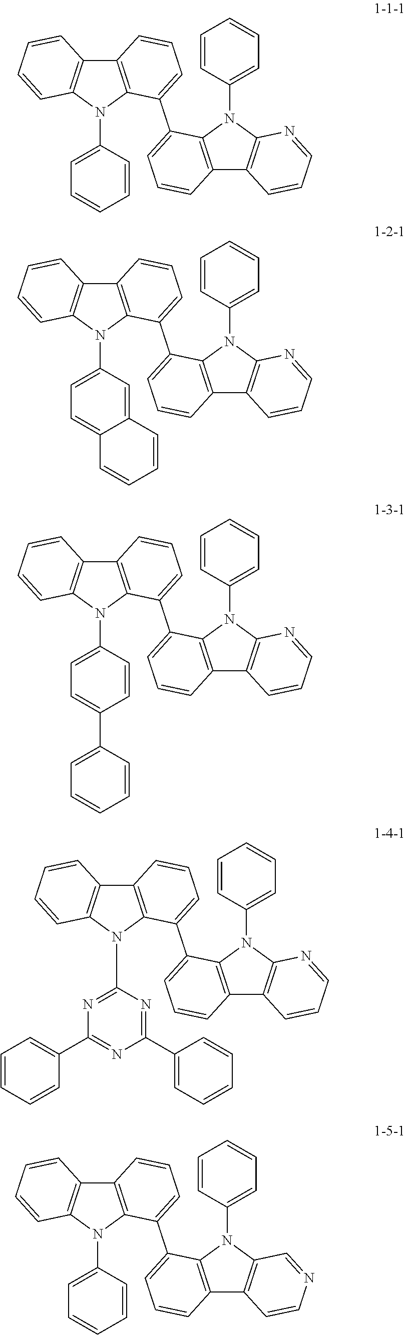

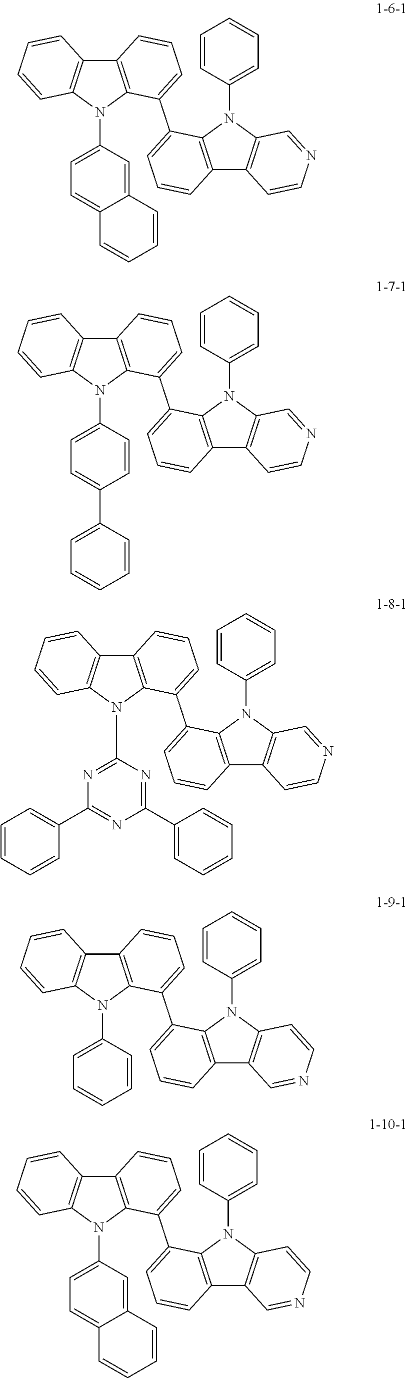

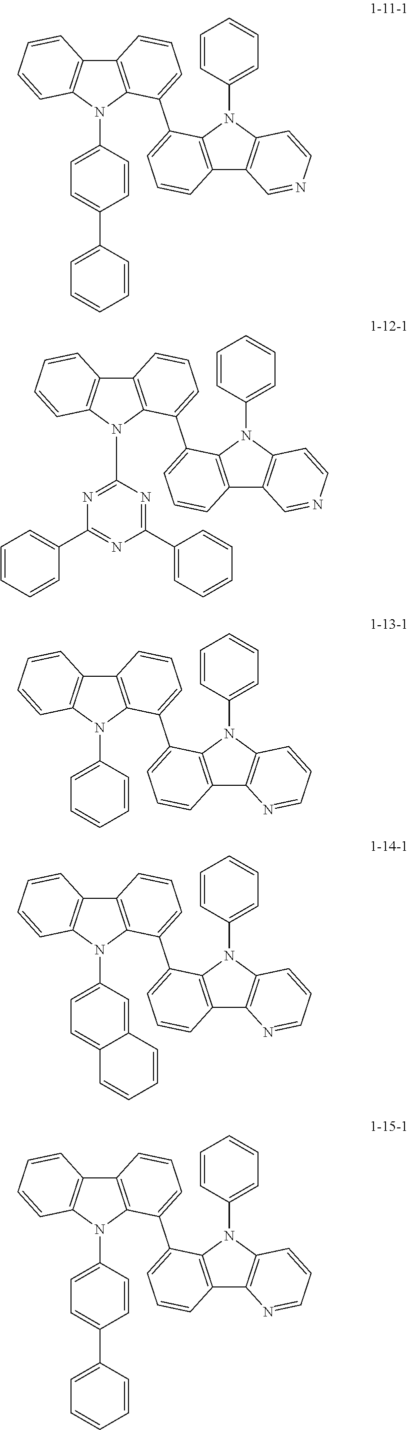

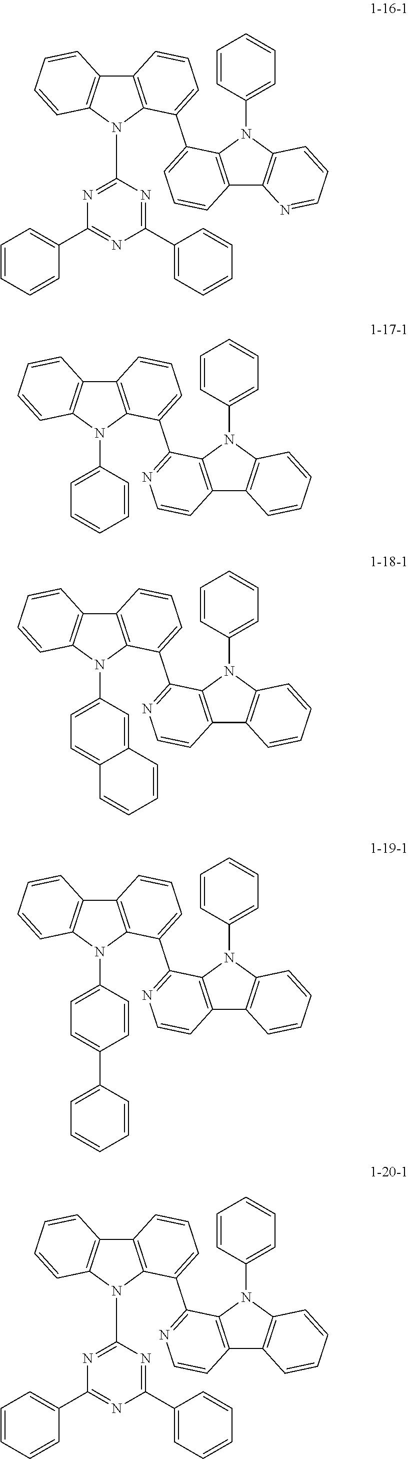 [SMILES: c1ccc(-c2nc(-c3ccccc3)nc(-n3c4ccccc4c4cccc(-c5cccc6c7ncccc7n(-c7ccccc7)c56)c43)n2)cc1 ; c1ccc(-n2c3ccccc3c3cccc(-c4nccc5c6ccccc6n(-c6ccccc6)c45)c32)cc1 ; c1ccc(-n2c3ccccc3c3ccnc(-c4cccc5c6ccccc6n(-c6ccc7ccccc7c6)c45)c32)cc1 ; c1ccc(-c2ccc(-n3c4ccccc4c4cccc(-c5nccc6c7ccccc7n(-c7ccccc7)c56)c43)cc2)cc1 ; c1ccc(-c2nc(-c3ccccc3)nc(-n3c4ccccc4c4cccc(-c5nccc6c7ccccc7n(-c7ccccc7)c56)c43)n2)cc1]

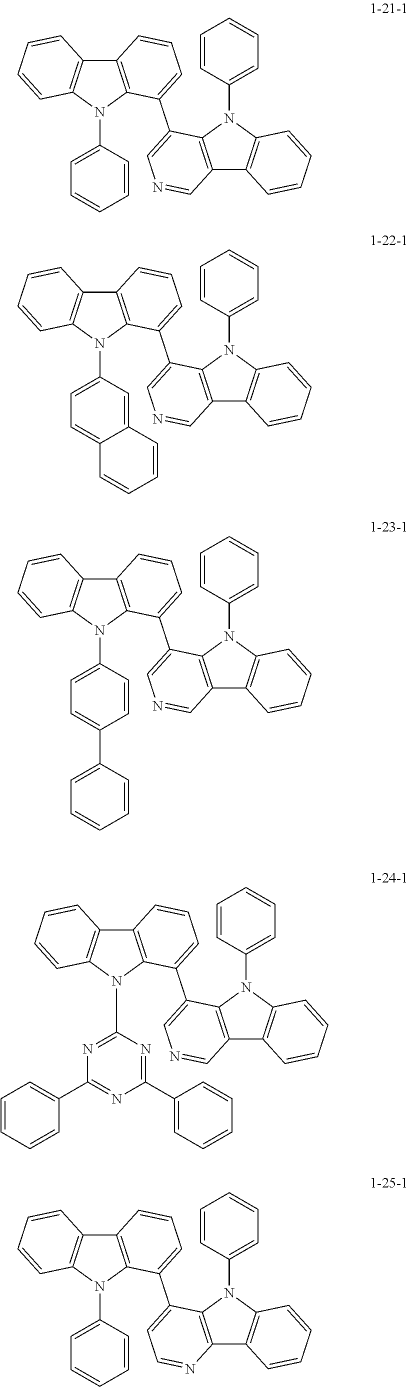 [SMILES: c1ccc(-n2c3ccccc3c3cccc(-c4cncc5c6ccccc6n(-c6ccccc6)c45)c32)cc1 ; c1ccc(-n2c3ccccc3c3cncc(-c4cccc5c6ccccc6n(-c6ccc7ccccc7c6)c45)c32)cc1 ; c1ccc(-c2ccc(-n3c4ccccc4c4cccc(-c5cncc6c7ccccc7n(-c7ccccc7)c56)c43)cc2)cc1 ; c1ccc(-c2nc(-c3ccccc3)nc(-n3c4ccccc4c4cccc(-c5cncc6c7ccccc7n(-c7ccccc7)c56)c43)n2)cc1 ; c1ccc(-n2c3ccccc3c3cccc(-c4ccnc5c6ccccc6n(-c6ccccc6)c45)c32)cc1]

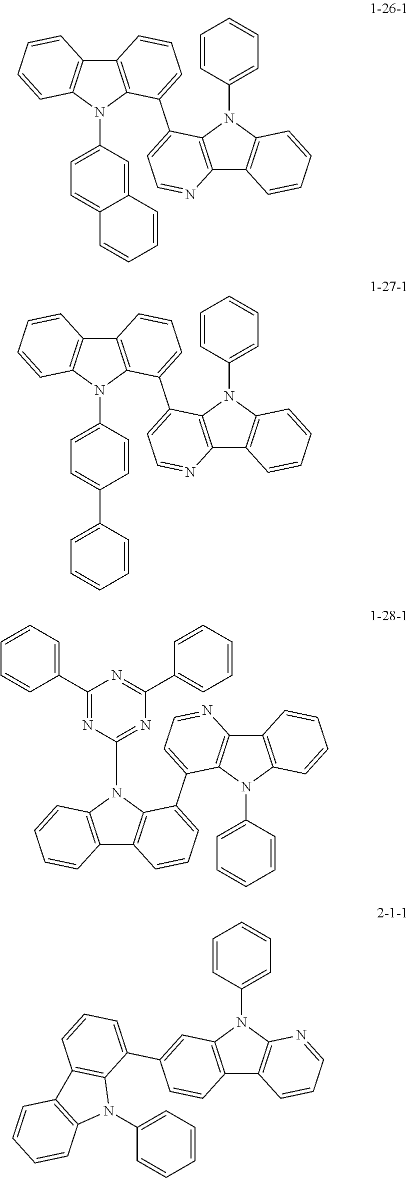

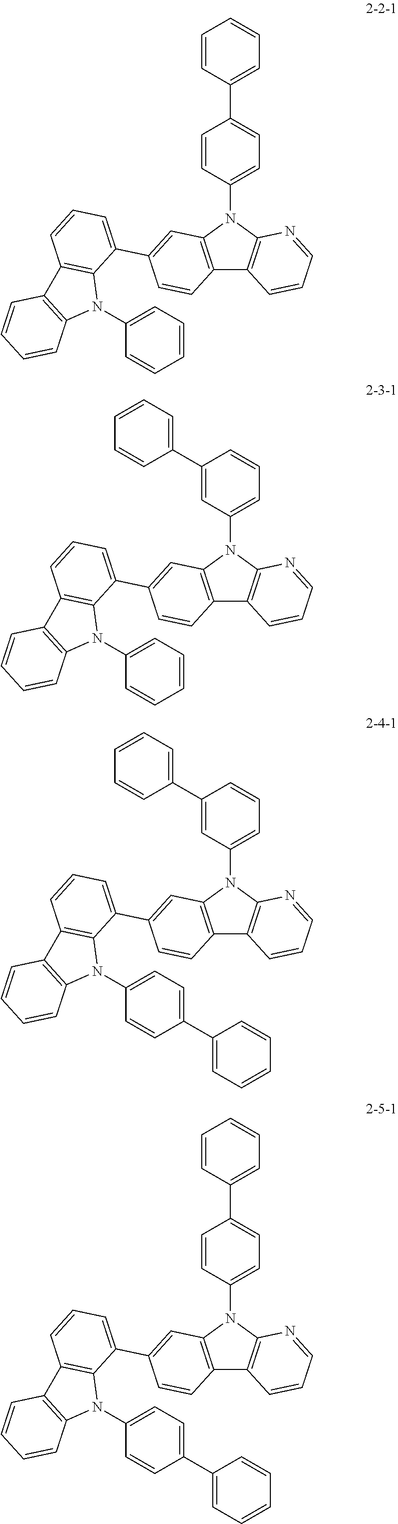

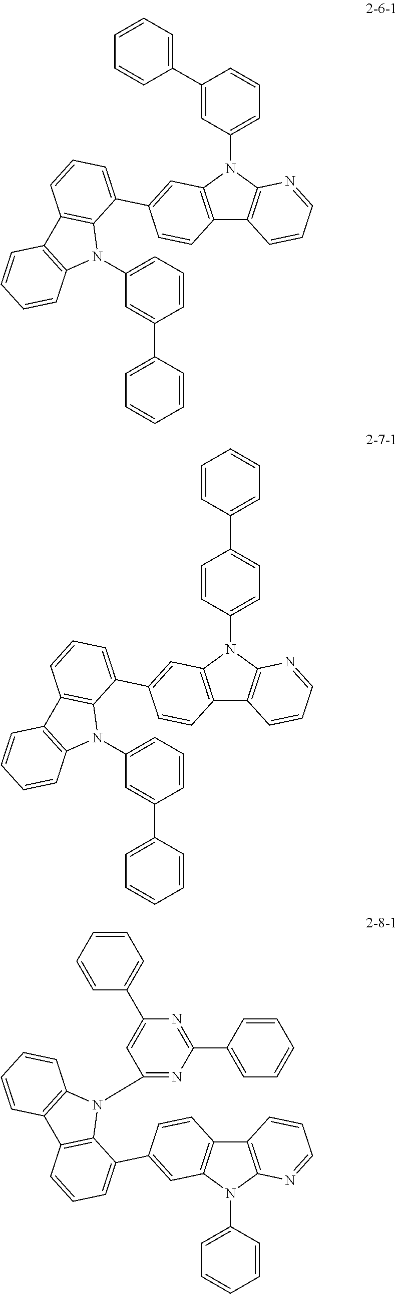

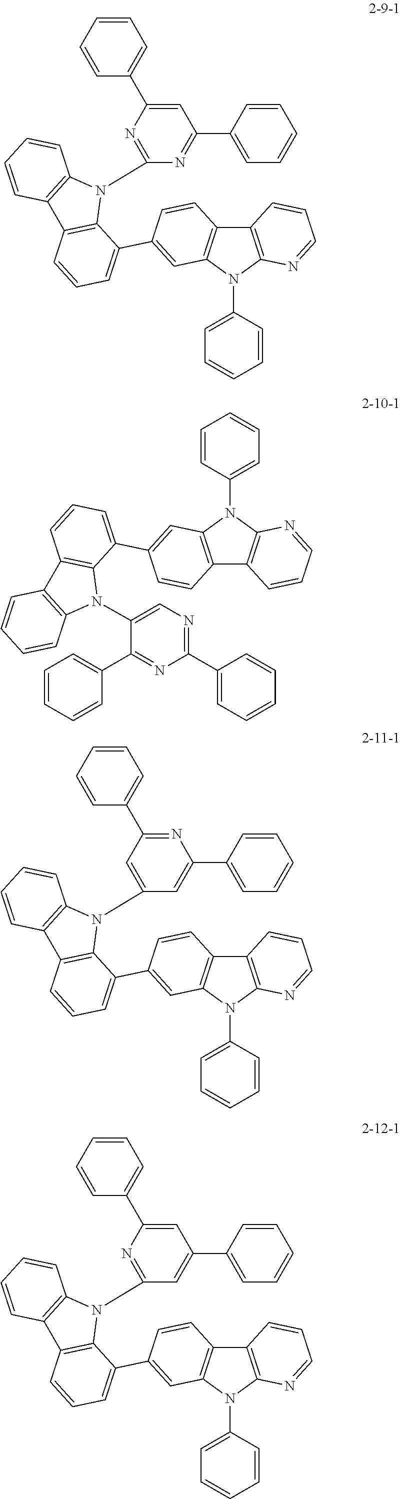

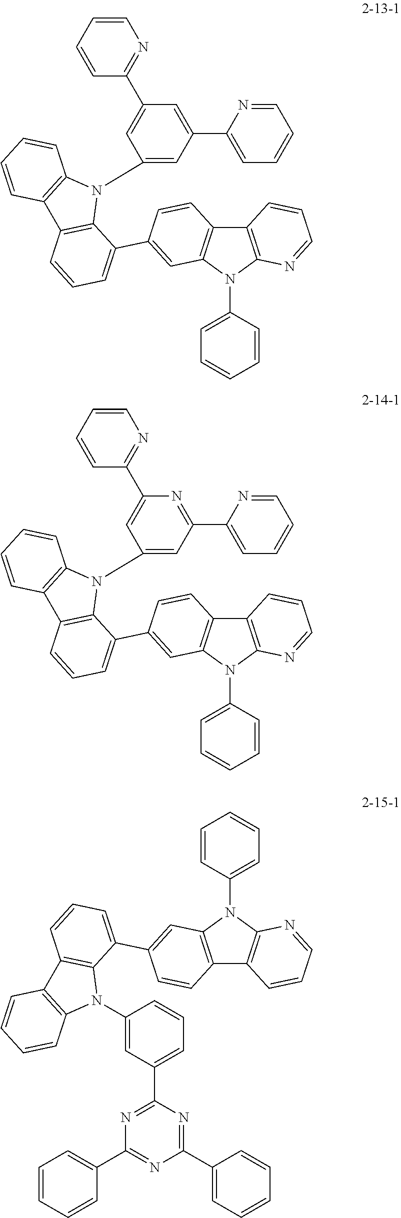

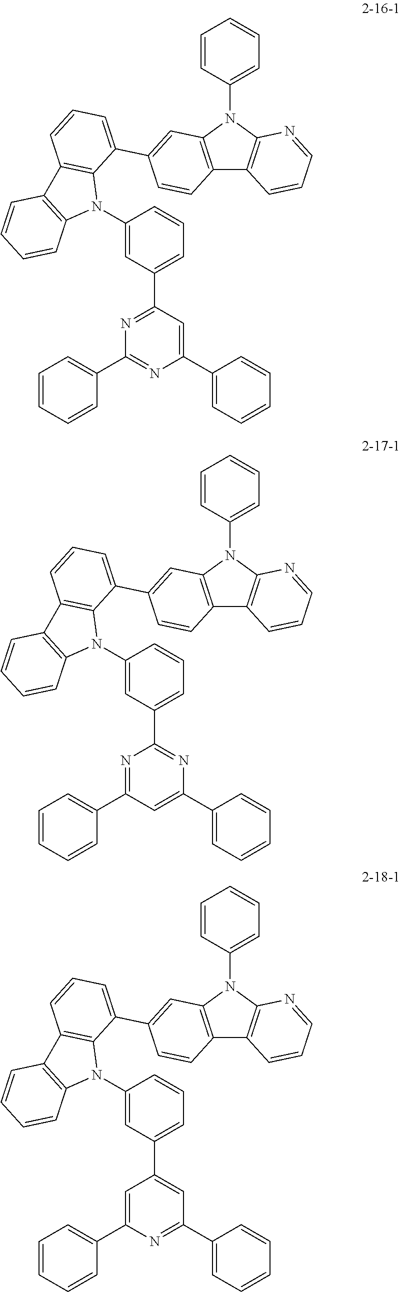

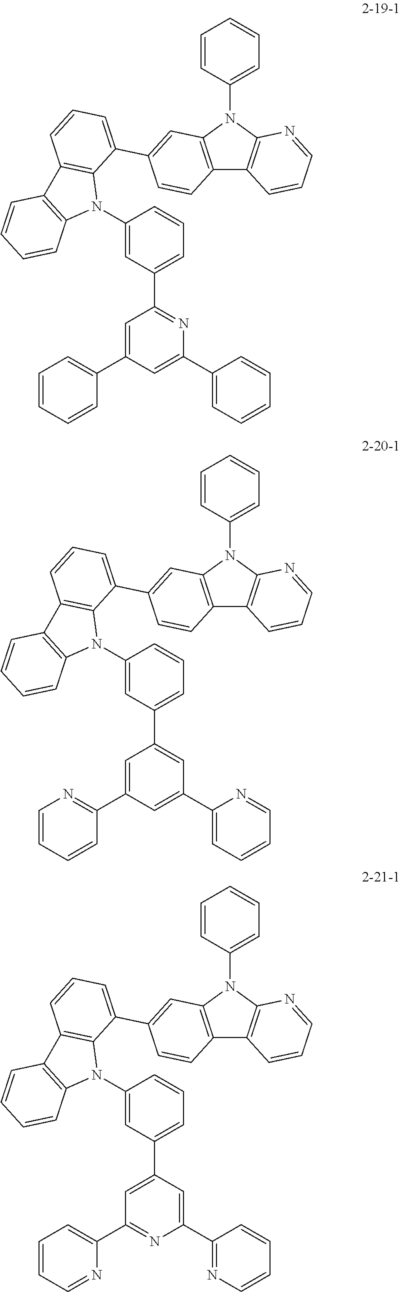

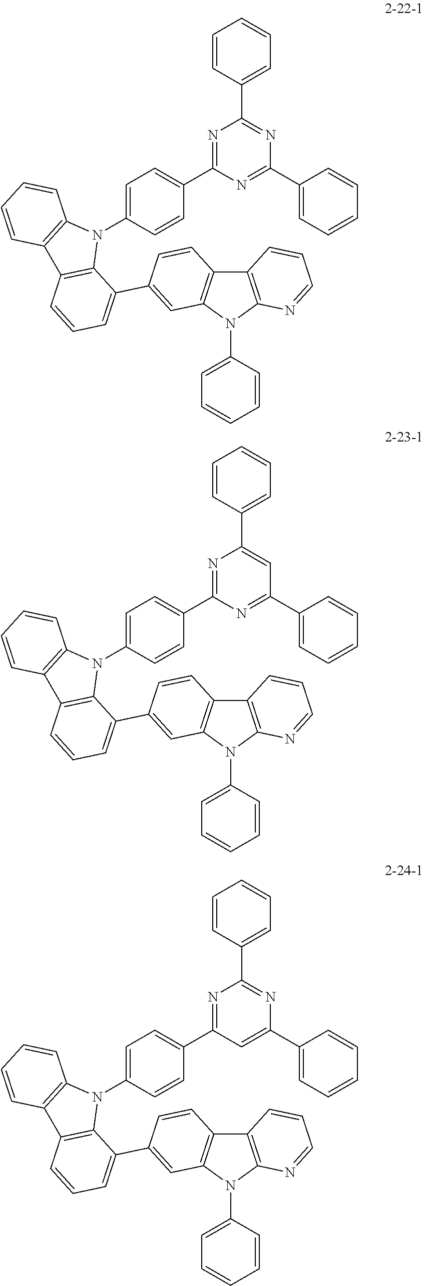

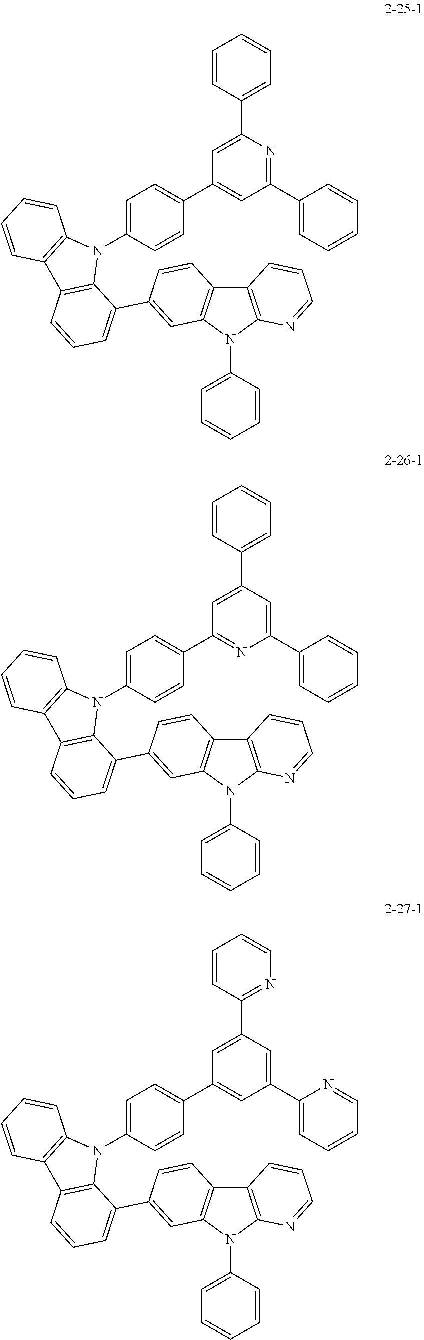

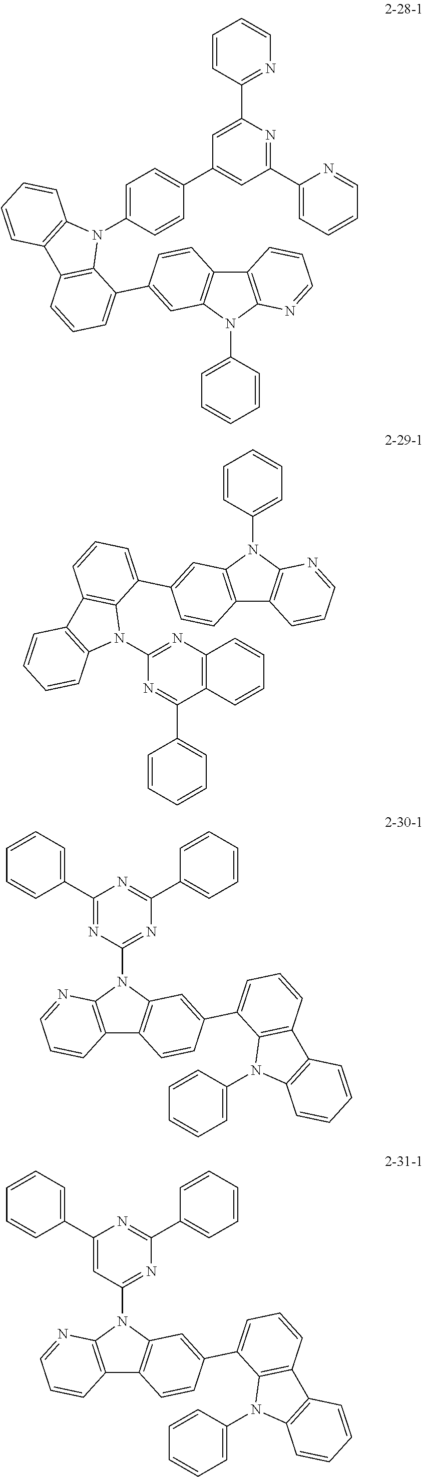

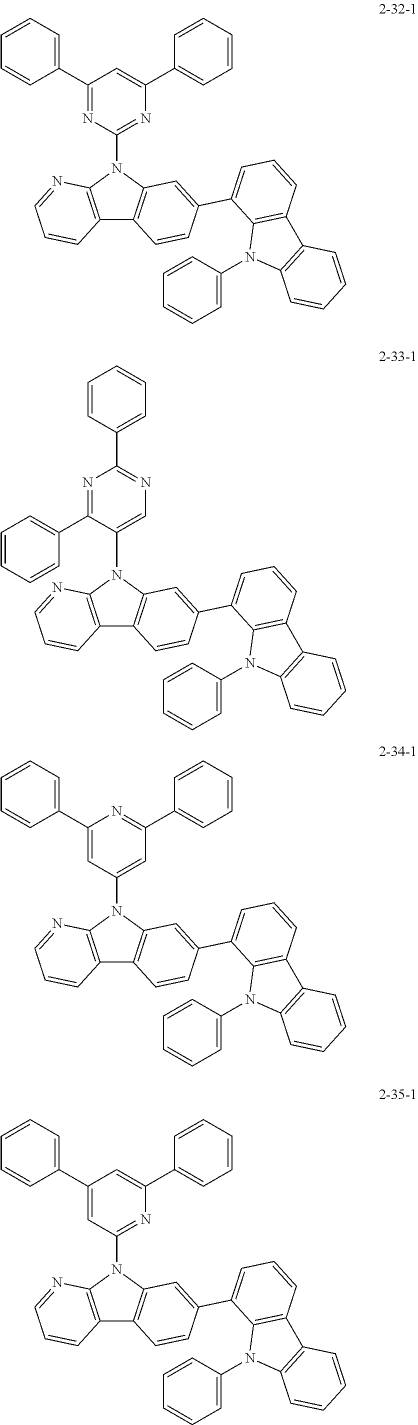

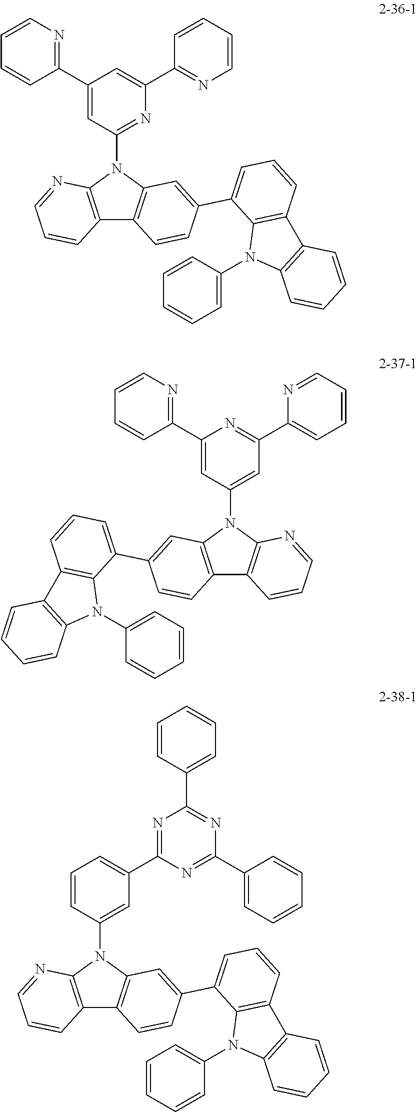

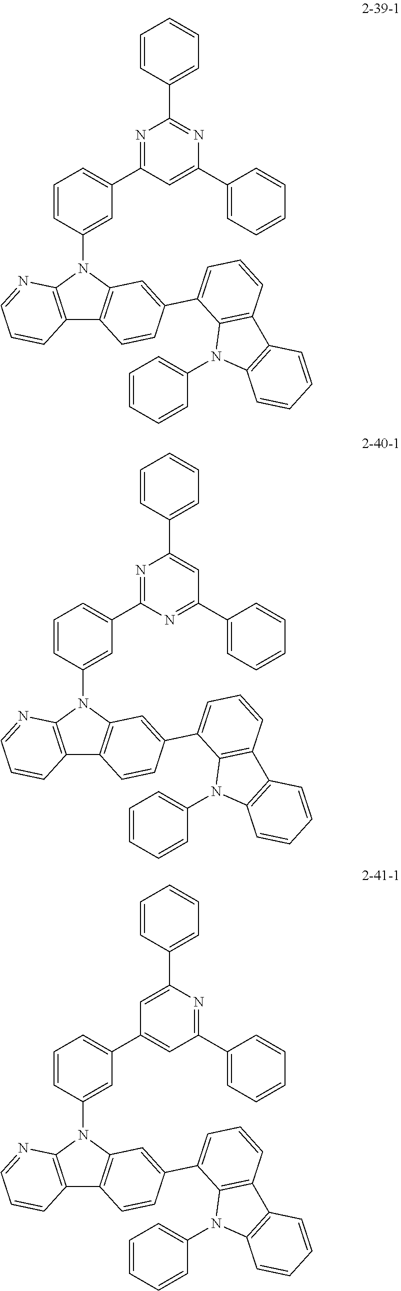

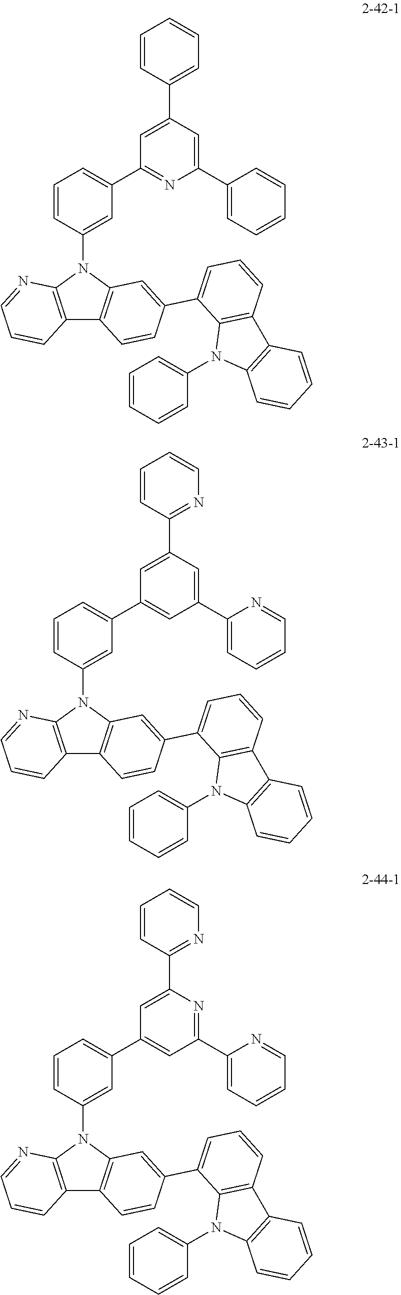

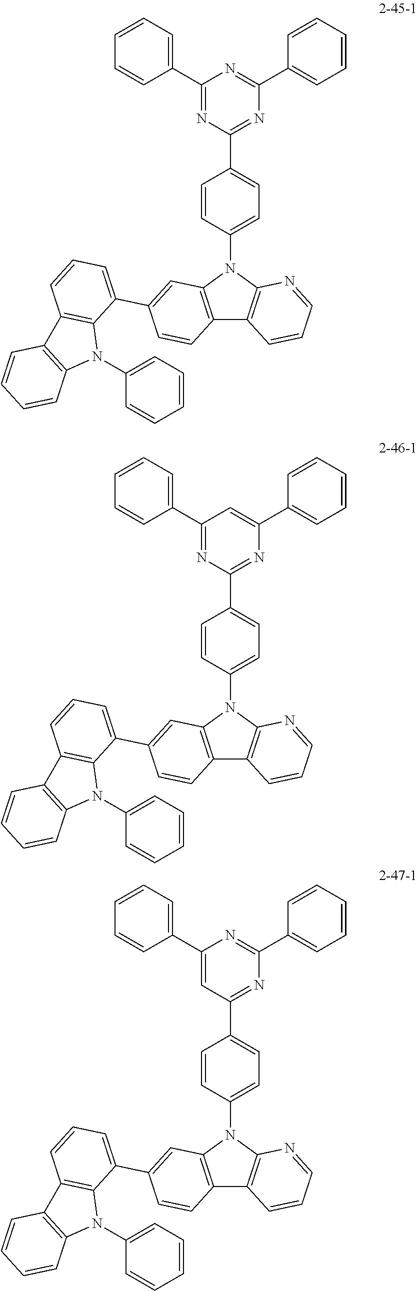

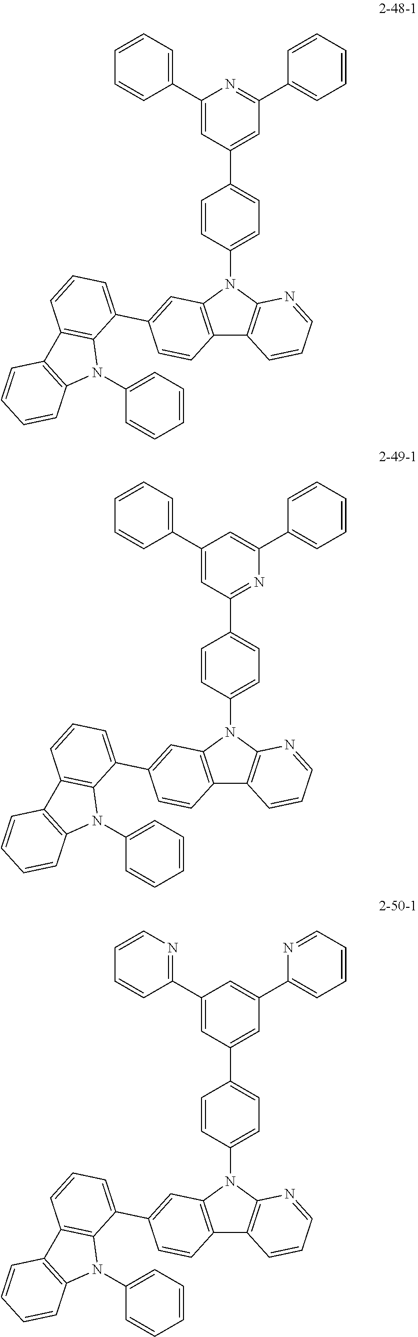

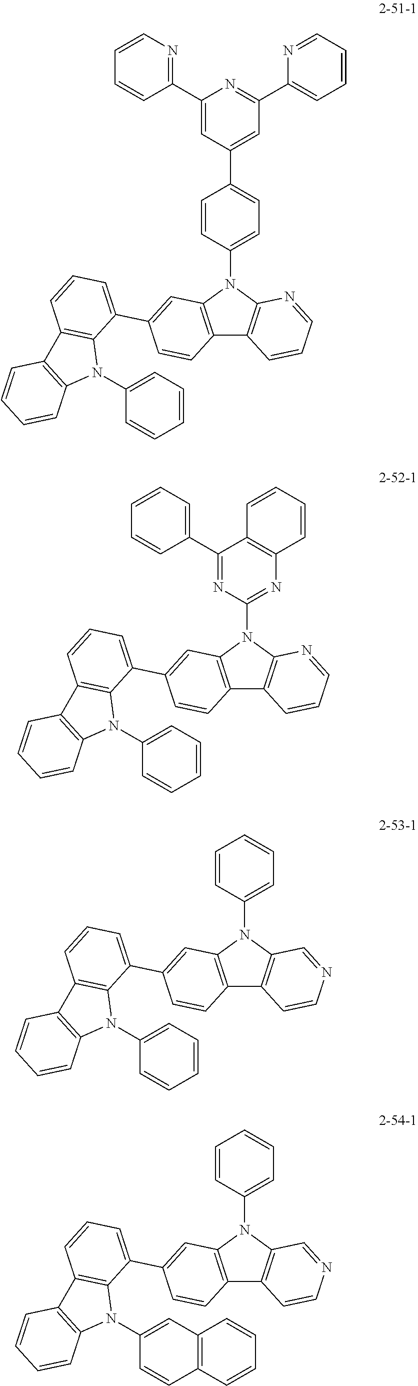 [SMILES: c1ccc(-n2c3ccccc3c3cccc(-c4ccc5c6cccnc6n(-c6ccc(-c7cc(-c8ccccn8)nc(-c8ccccn8)c7)cc6)c5c4)c32)cc1 ; c1ccc(-c2nc(-n3c4cc(-c5cccc6c7ccccc7n(-c7ccccc7)c56)ccc4c4cccnc43)nc3ccccc23)cc1 ; c1ccc(-n2c3cnccc3c3ccc(-c4cccc5c6ccccc6n(-c6ccccc6)c45)cc32)cc1 ; c1ccc(-n2c3cnccc3c3ccc(-c4cccc5c6ccccc6n(-c6ccc7ccccc7c6)c45)cc32)cc1]

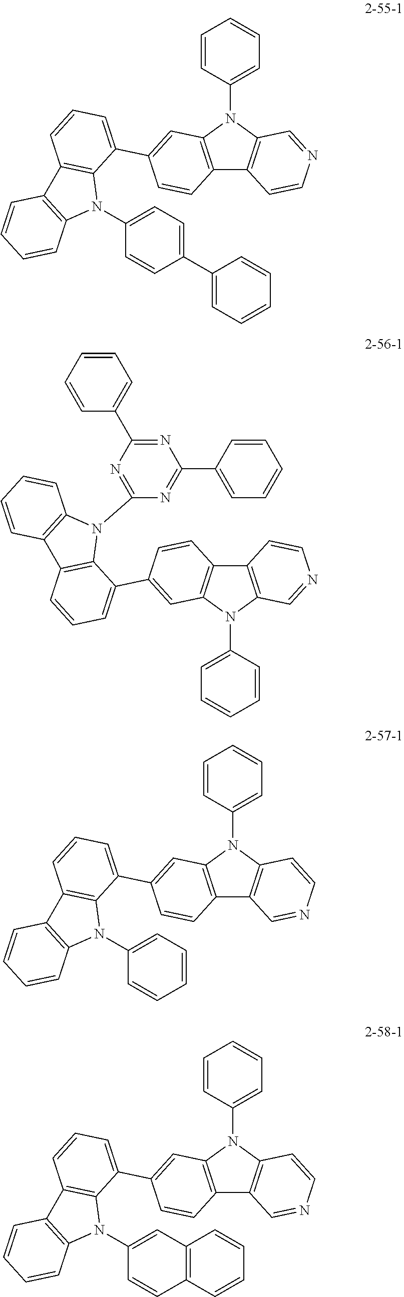

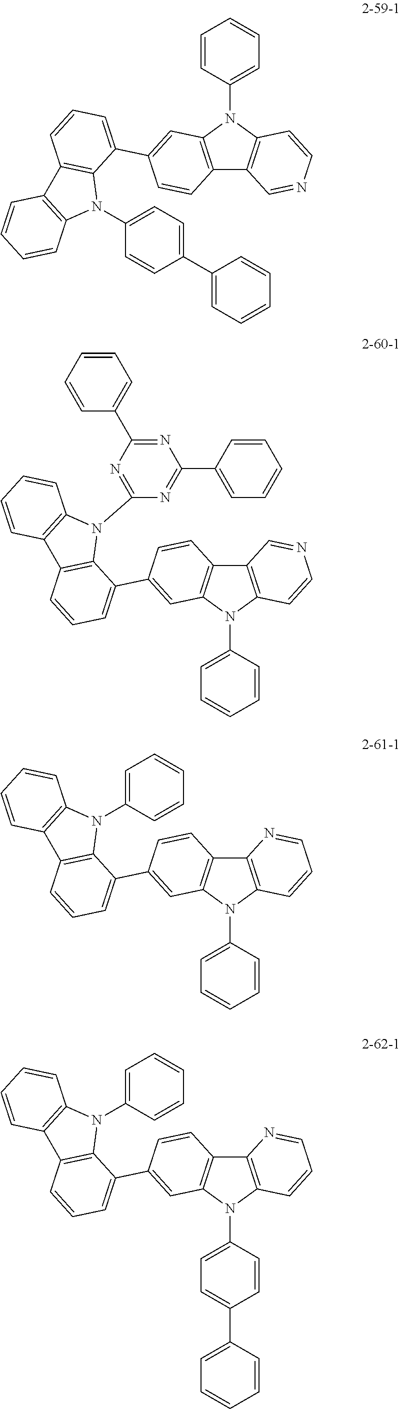 [SMILES: c1ccc(-c2ccc(-n3c4ccccc4c4cccc(-c5ccc6c7cnccc7n(-c7ccccc7)c6c5)c43)cc2)cc1 ; c1ccc(-c2nc(-c3ccccc3)nc(-n3c4ccccc4c4cccc(-c5ccc6c7cnccc7n(-c7ccccc7)c6c5)c43)n2)cc1 ; c1ccc(-n2c3cc(-c4cccc5c6ccccc6n(-c6ccccc6)c45)ccc3c3ncccc32)cc1 ; c1ccc(-c2ccc(-n3c4cc(-c5cccc6c7ccccc7n(-c7ccccc7)c56)ccc4c4ncccc43)cc2)cc1]

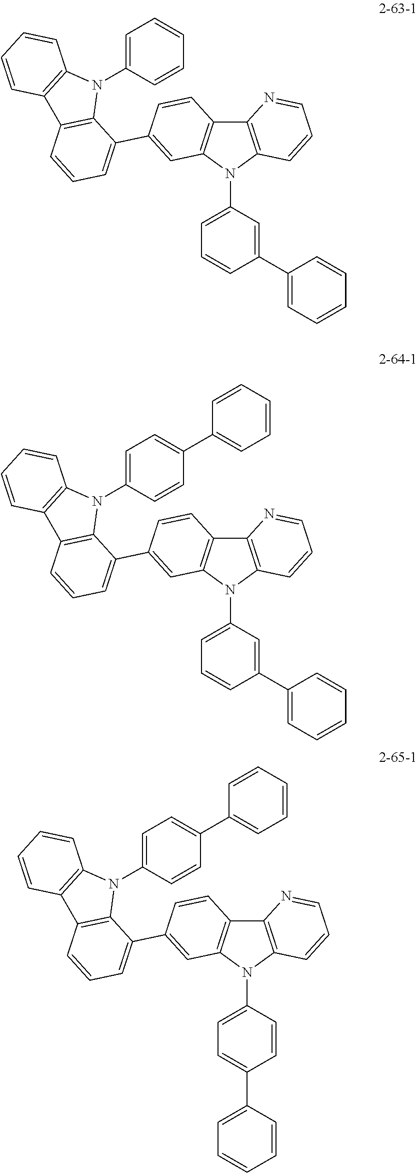 [SMILES: c1ccc(-c2cccc(-n3c4cc(-c5cccc6c7ccccc7n(-c7ccccc7)c56)ccc4c4ncccc43)c2)cc1 ; c1ccc(-c2ccc(-n3c4ccccc4c4cccc(-c5ccc6c7ncccc7n(-c7cccc(-c8ccccc8)c7)c6c5)c43)cc2)cc1 ; c1ccc(-c2ccc(-n3c4cc(-c5cccc6c7ccccc7n(-c7ccc(-c8ccccc8)cc7)c56)ccc4c4ncccc43)cc2)cc1]

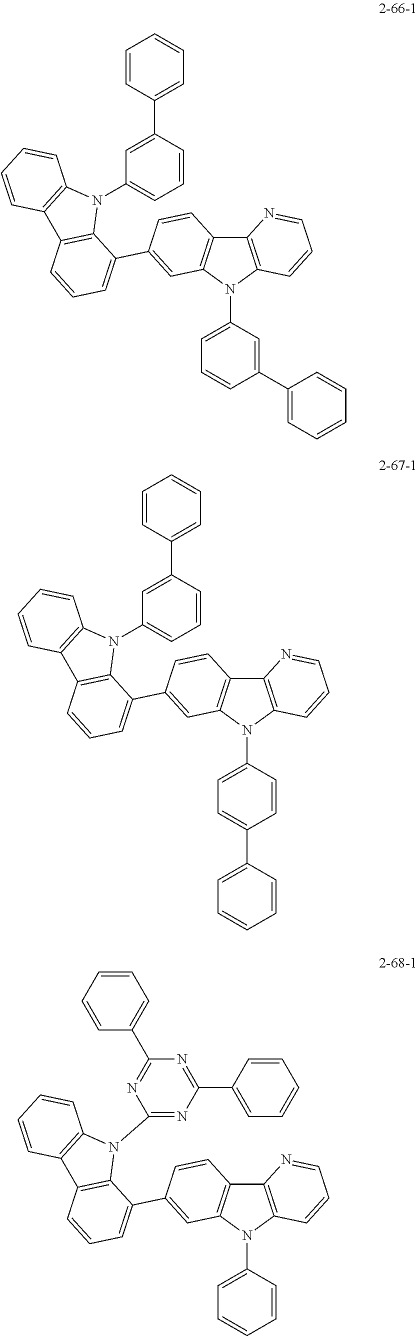 [SMILES: c1ccc(-c2cccc(-n3c4cc(-c5cccc6c7ccccc7n(-c7cccc(-c8ccccc8)c7)c56)ccc4c4ncccc43)c2)cc1 ; c1ccc(-c2ccc(-n3c4cc(-c5cccc6c7ccccc7n(-c7cccc(-c8ccccc8)c7)c56)ccc4c4ncccc43)cc2)cc1 ; c1ccc(-c2nc(-c3ccccc3)nc(-n3c4ccccc4c4cccc(-c5ccc6c7ncccc7n(-c7ccccc7)c6c5)c43)n2)cc1]

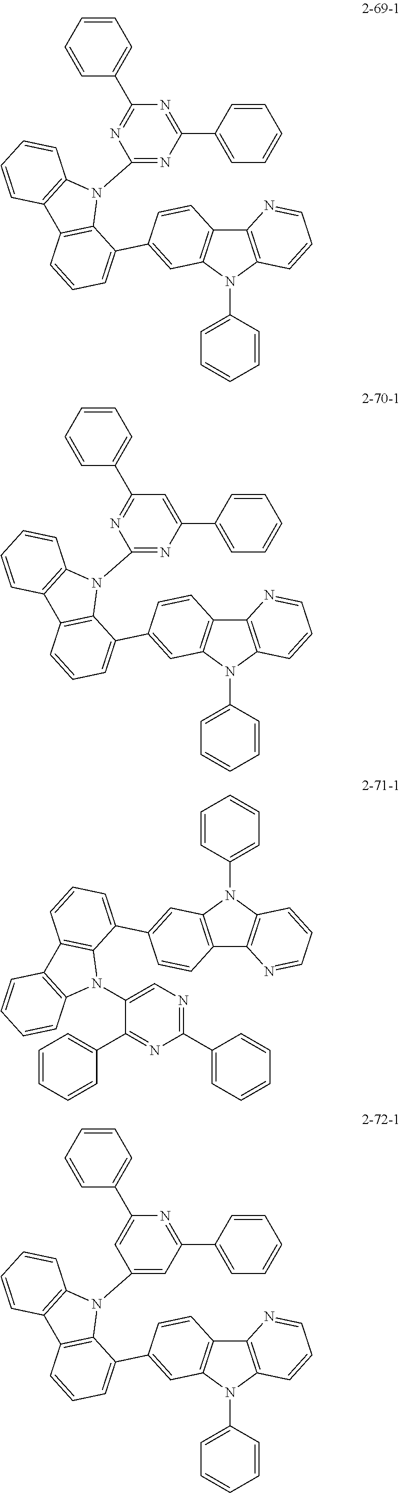

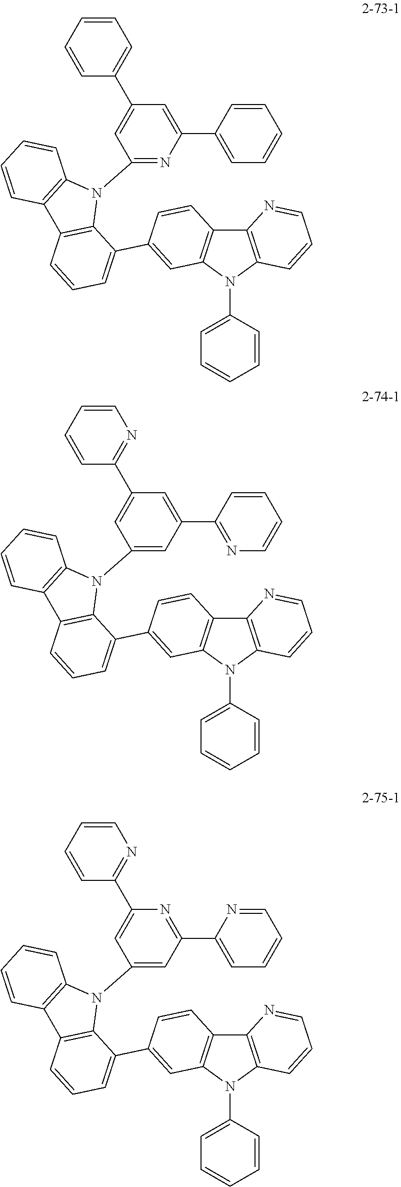 [SMILES: c1ccc(-c2cc(-c3ccccc3)nc(-n3c4ccccc4c4cccc(-c5ccc6c7ncccc7n(-c7ccccc7)c6c5)c43)c2)cc1 ; c1ccc(-n2c3cc(-c4cccc5c6ccccc6n(-c6cc(-c7ccccn7)cc(-c7ccccn7)c6)c45)ccc3c3ncccc32)cc1 ; c1ccc(-n2c3cc(-c4cccc5c6ccccc6n(-c6cc(-c7ccccn7)nc(-c7ccccn7)c6)c45)ccc3c3ncccc32)cc1]

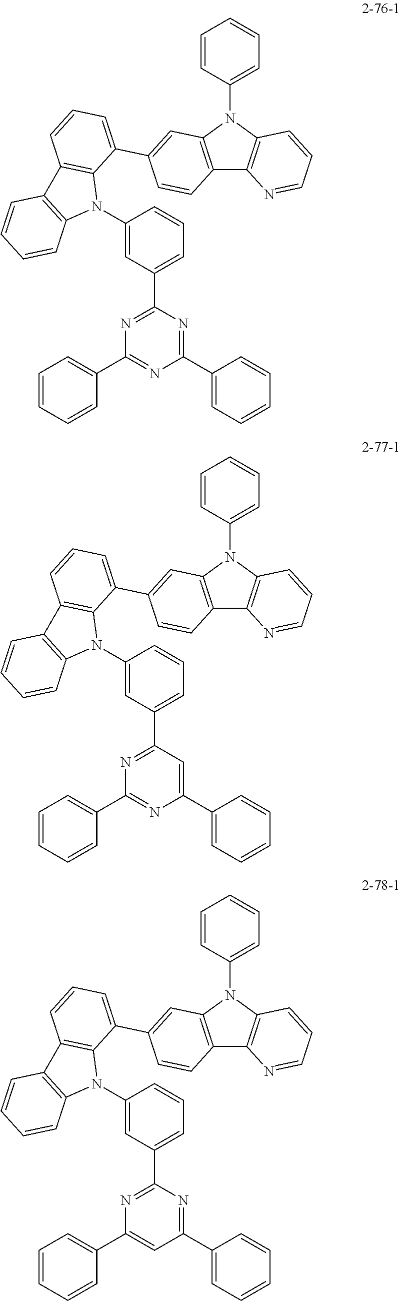

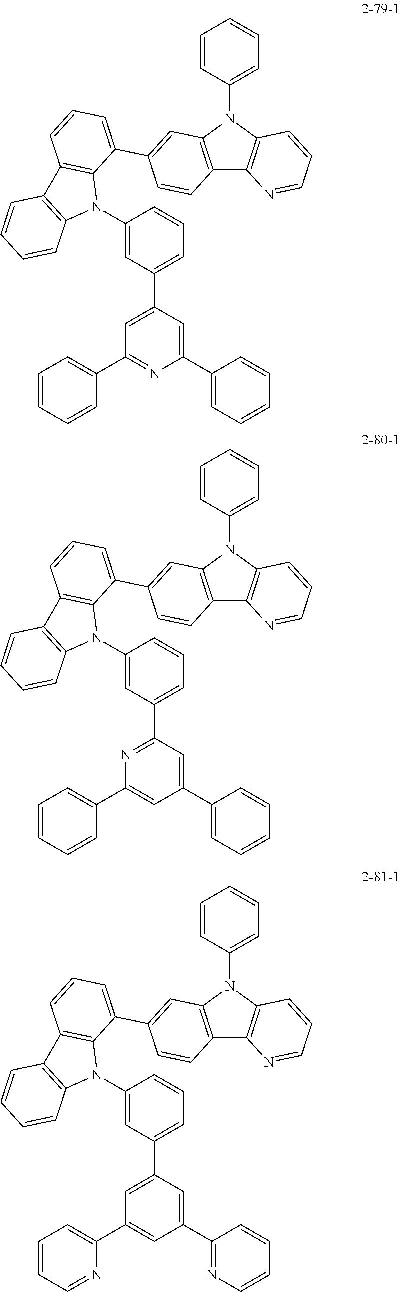

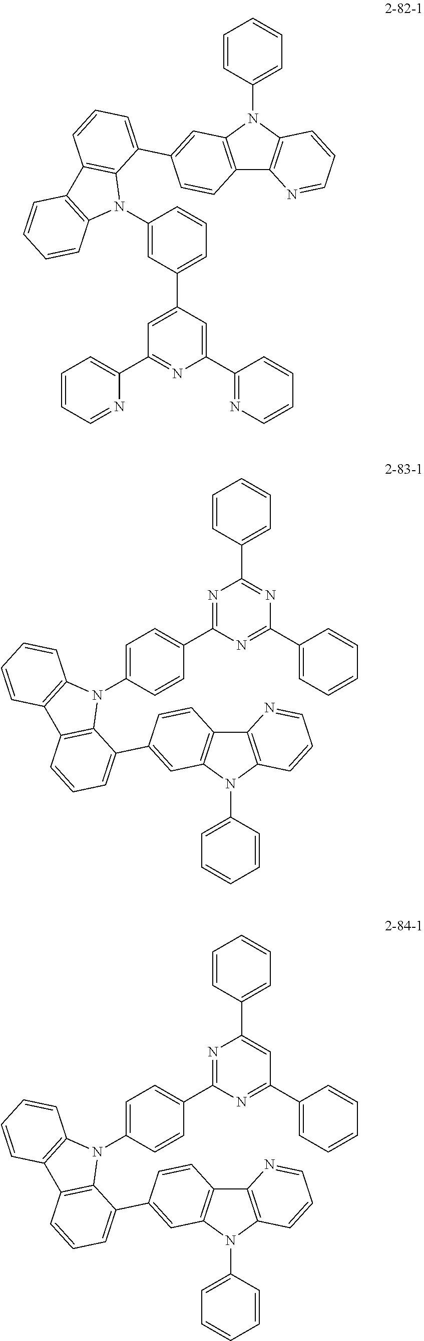 [SMILES: c1ccc(-n2c3cc(-c4cccc5c6ccccc6n(-c6cccc(-c7cc(-c8ccccn8)nc(-c8ccccn8)c7)c6)c45)ccc3c3ncccc32)cc1 ; c1ccc(-c2nc(-c3ccccc3)nc(-c3ccc(-n4c5ccccc5c5cccc(-c6ccc7c8ncccc8n(-c8ccccc8)c7c6)c54)cc3)n2)cc1 ; c1ccc(-c2cc(-c3ccccc3)nc(-c3ccc(-n4c5ccccc5c5cccc(-c6ccc7c8ncccc8n(-c8ccccc8)c7c6)c54)cc3)n2)cc1]

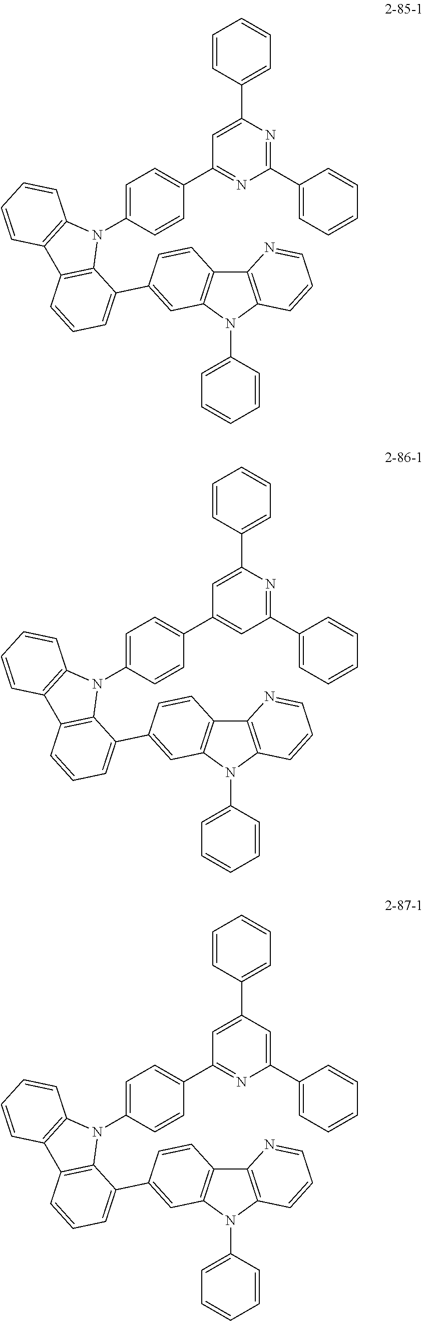

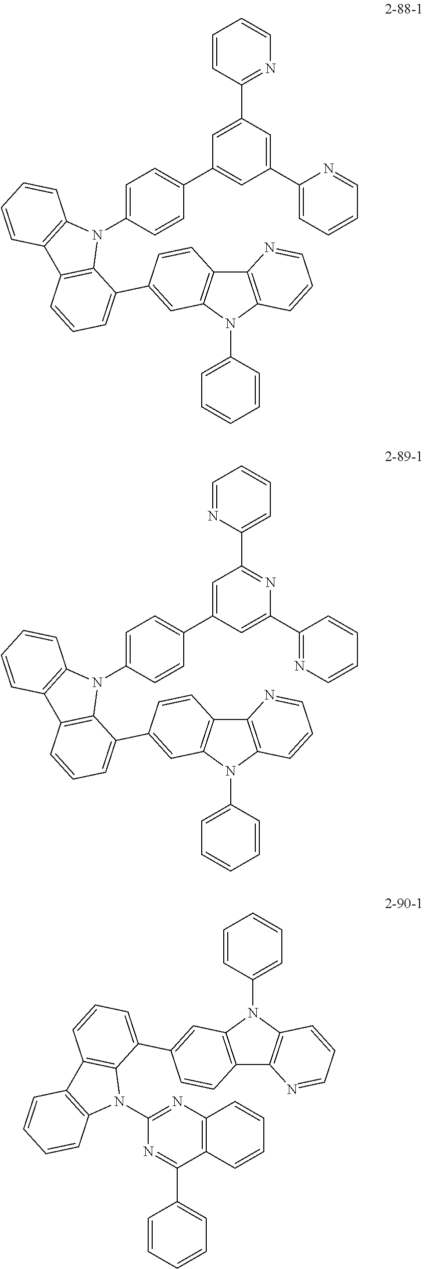

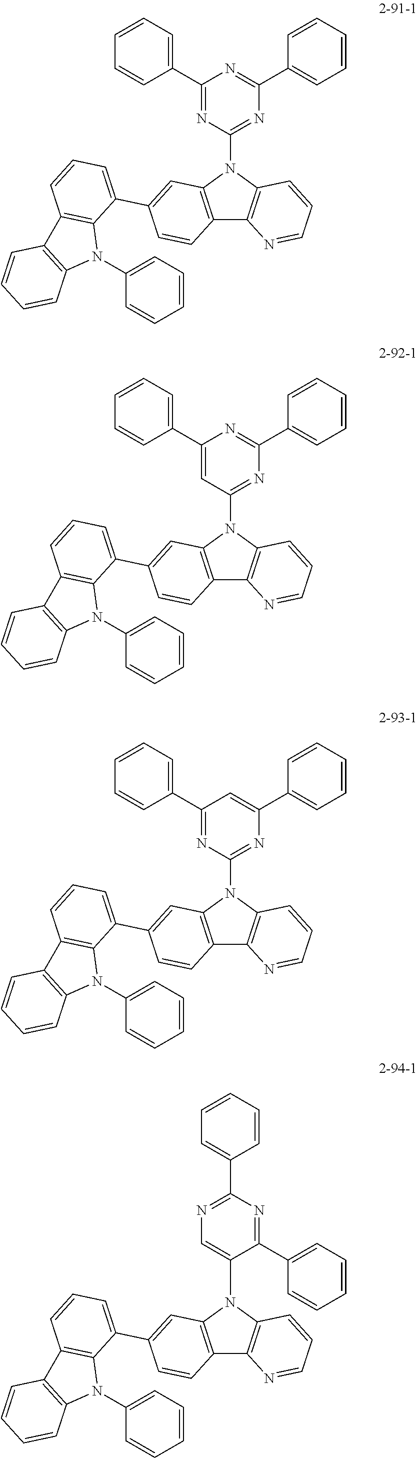 [SMILES: c1ccc(-c2nc(-c3ccccc3)nc(-n3c4cc(-c5cccc6c7ccccc7n(-c7ccccc7)c56)ccc4c4ncccc43)n2)cc1 ; c1ccc(-c2cc(-n3c4cc(-c5cccc6c7ccccc7n(-c7ccccc7)c56)ccc4c4ncccc43)nc(-c3ccccc3)n2)cc1 ; c1ccc(-c2cc(-c3ccccc3)nc(-n3c4cc(-c5cccc6c7ccccc7n(-c7ccccc7)c56)ccc4c4ncccc43)n2)cc1 ; c1ccc(-c2ncc(-n3c4cc(-c5cccc6c7ccccc7n(-c7ccccc7)c56)ccc4c4ncccc43)c(-c3ccccc3)n2)cc1]

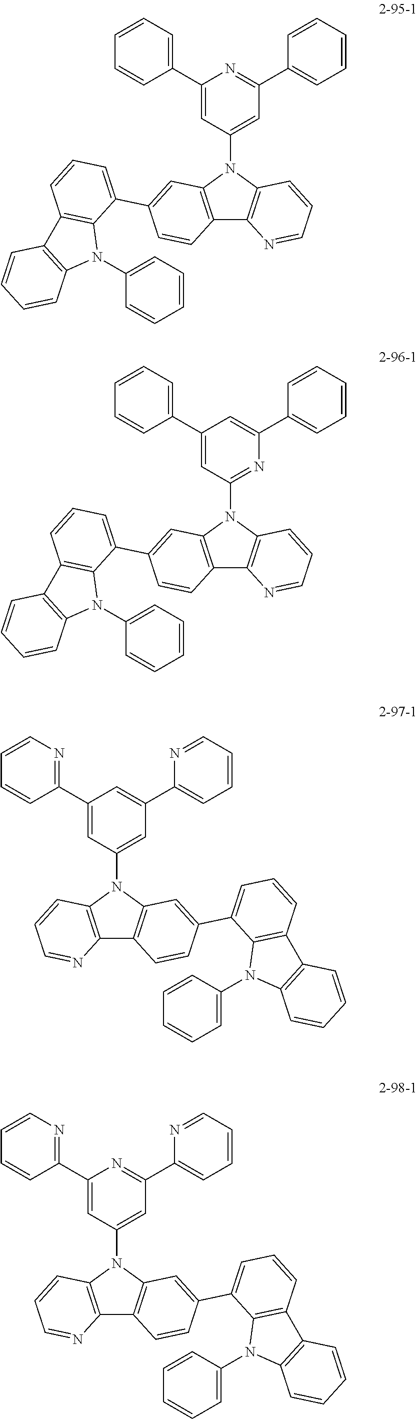

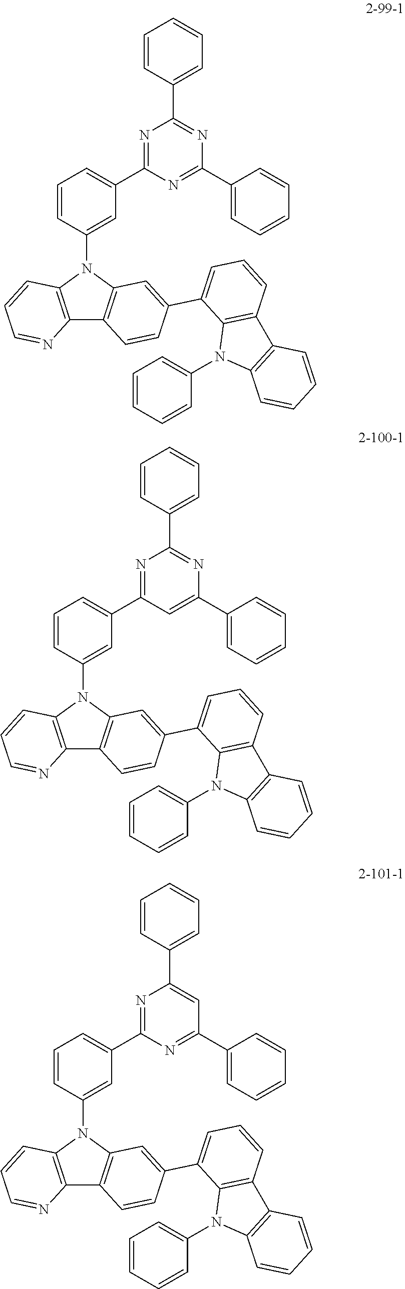

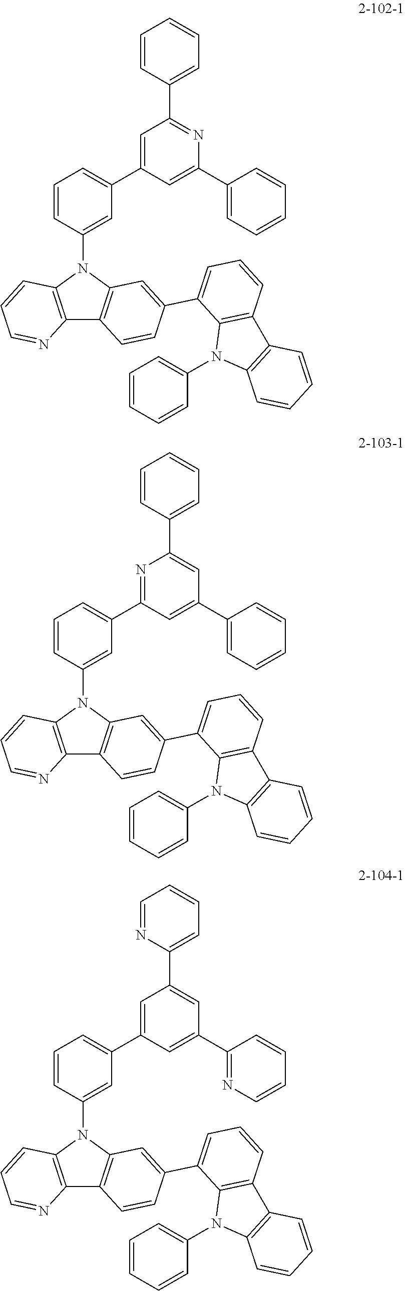

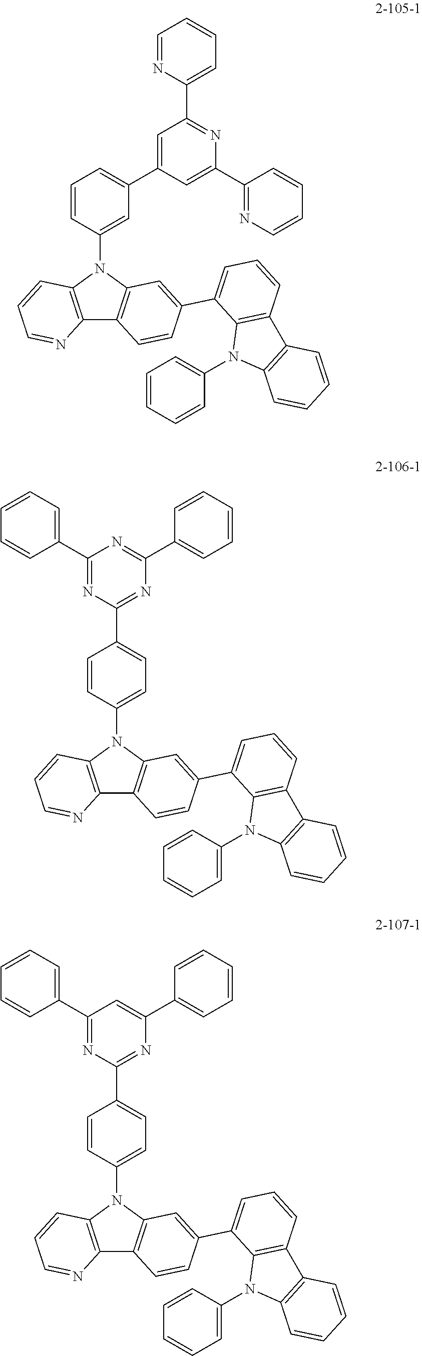 [SMILES: c1ccc(-n2c3ccccc3c3cccc(-c4ccc5c6ncccc6n(-c6cccc(-c7cc(-c8ccccn8)nc(-c8ccccn8)c7)c6)c5c4)c32)cc1 ; c1ccc(-c2nc(-c3ccccc3)nc(-c3ccc(-n4c5cc(-c6cccc7c8ccccc8n(-c8ccccc8)c67)ccc5c5ncccc54)cc3)n2)cc1 ; c1ccc(-c2cc(-c3ccccc3)nc(-c3ccc(-n4c5cc(-c6cccc7c8ccccc8n(-c8ccccc8)c67)ccc5c5ncccc54)cc3)n2)cc1]

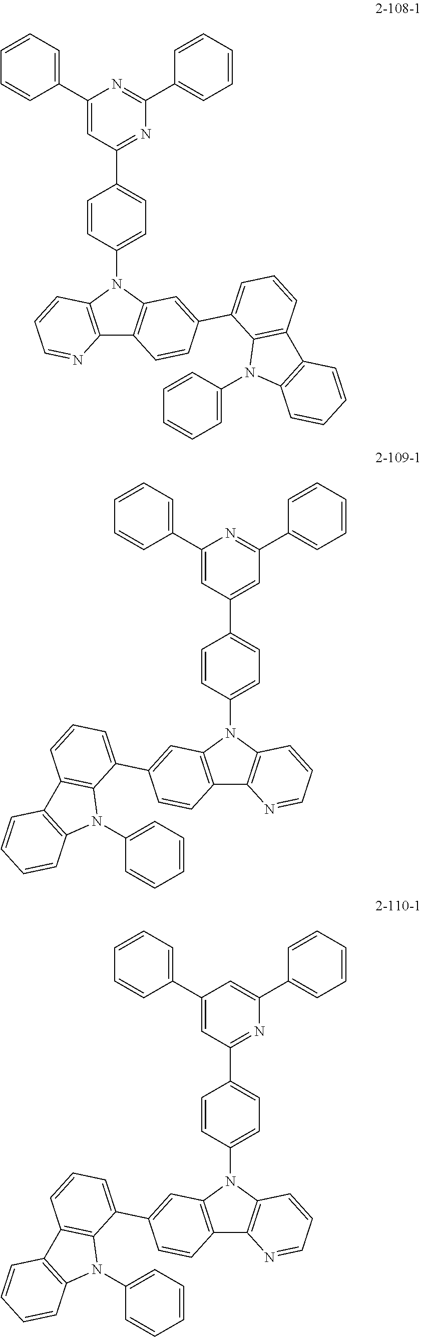

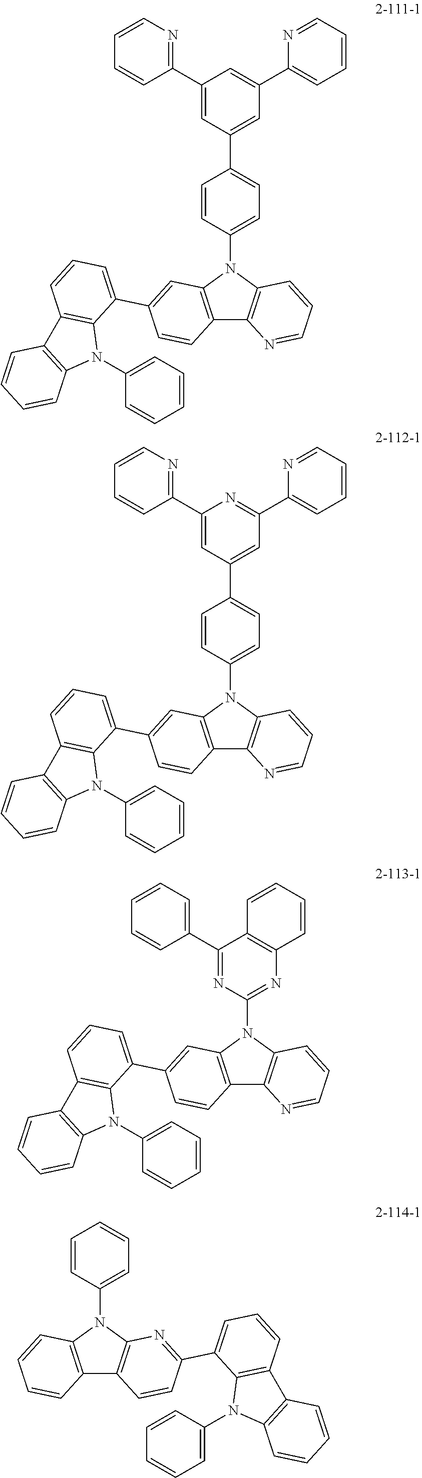

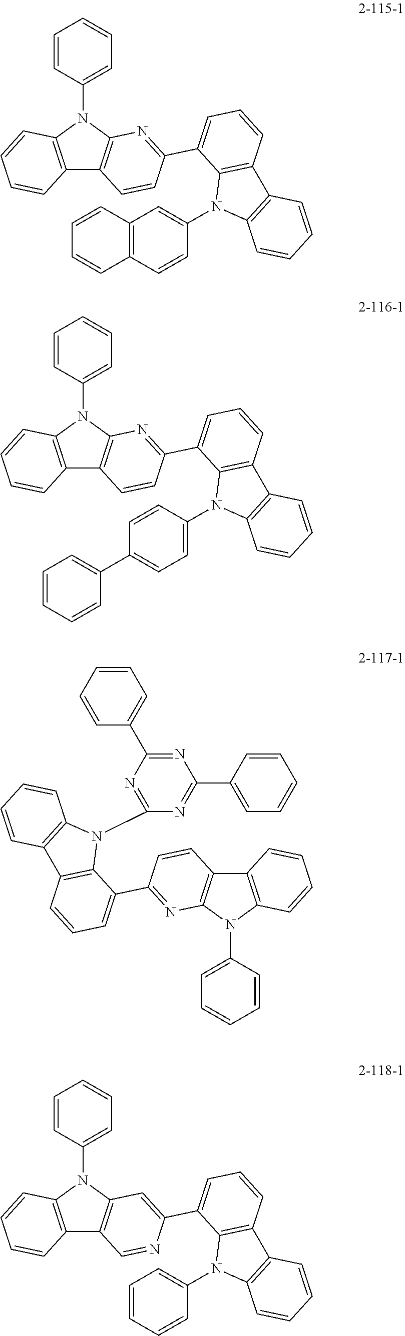 [SMILES: c1ccc(-n2c3ccccc3c3ccc(-c4cccc5c6ccccc6n(-c6ccc7ccccc7c6)c45)nc32)cc1 ; c1ccc(-c2ccc(-n3c4ccccc4c4cccc(-c5ccc6c7ccccc7n(-c7ccccc7)c6n5)c43)cc2)cc1 ; c1ccc(-c2nc(-c3ccccc3)nc(-n3c4ccccc4c4cccc(-c5ccc6c7ccccc7n(-c7ccccc7)c6n5)c43)n2)cc1 ; c1ccc(-n2c3ccccc3c3cnc(-c4cccc5c6ccccc6n(-c6ccccc6)c45)cc32)cc1]

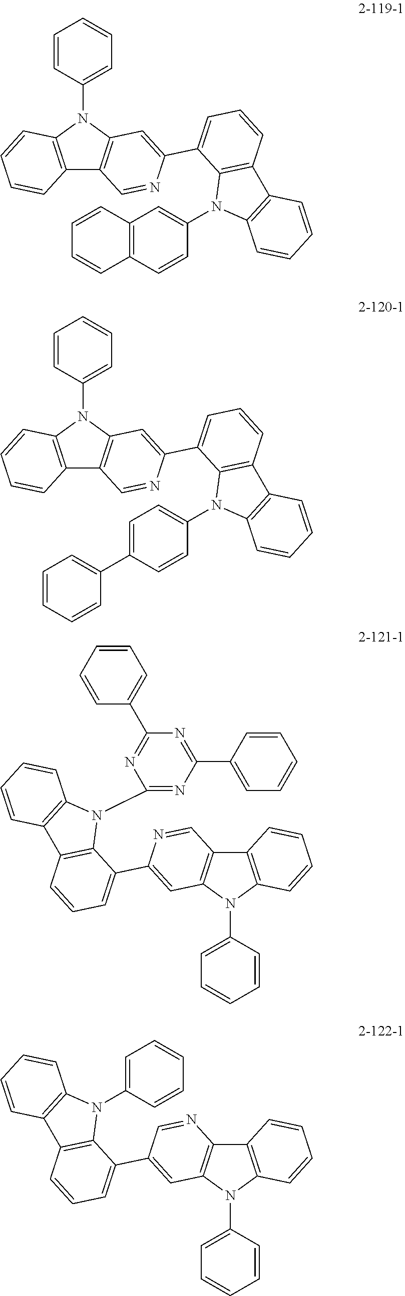

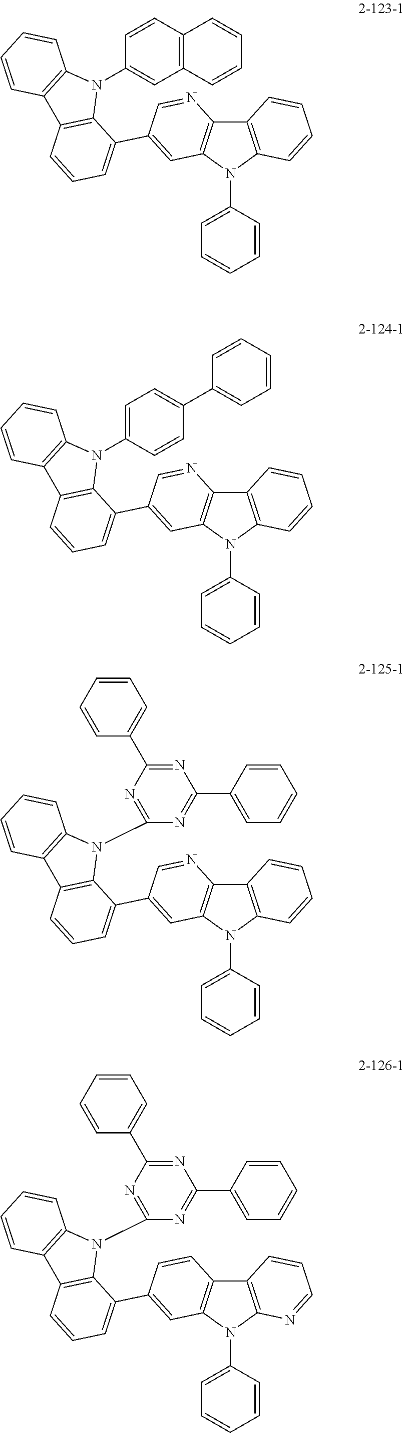 [SMILES: c1ccc(-n2c3ccccc3c3ncc(-c4cccc5c6ccccc6n(-c6ccc7ccccc7c6)c45)cc32)cc1 ; c1ccc(-c2ccc(-n3c4ccccc4c4cccc(-c5cnc6c7ccccc7n(-c7ccccc7)c6c5)c43)cc2)cc1 ; c1ccc(-c2nc(-c3ccccc3)nc(-n3c4ccccc4c4cccc(-c5cnc6c7ccccc7n(-c7ccccc7)c6c5)c43)n2)cc1 ; c1ccc(-c2nc(-c3ccccc3)nc(-n3c4ccccc4c4cccc(-c5ccc6c7cccnc7n(-c7ccccc7)c6c5)c43)n2)cc1]

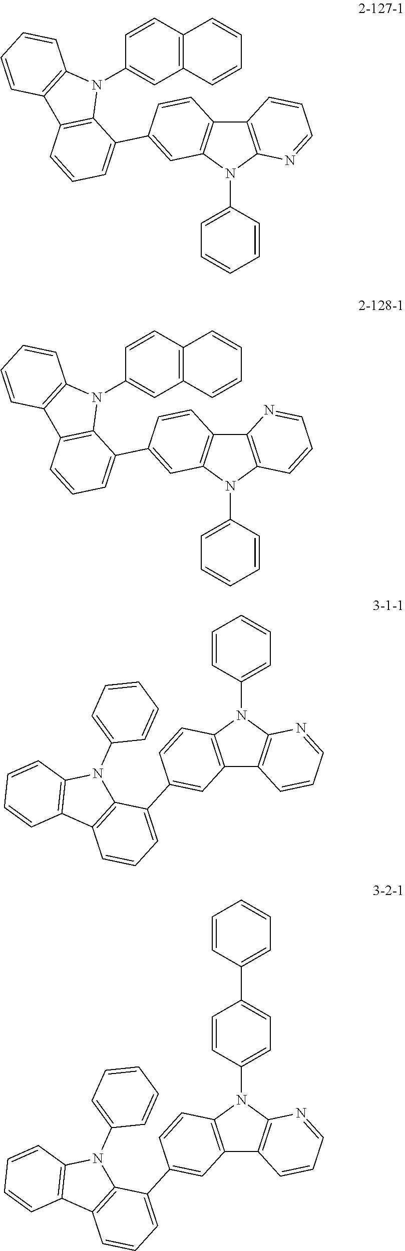

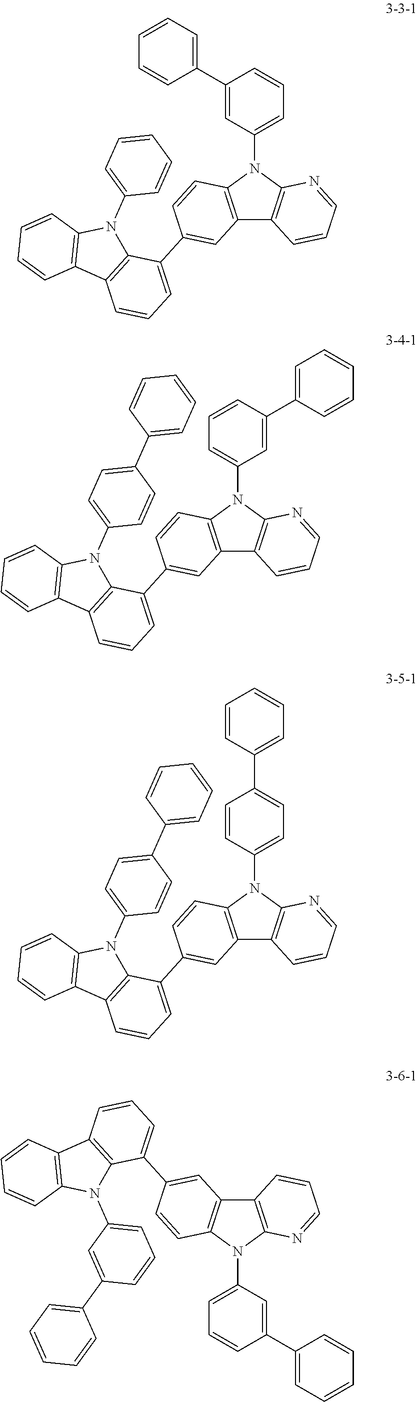 [SMILES: c1ccc(-c2cccc(-n3c4ccc(-c5cccc6c7ccccc7n(-c7ccccc7)c56)cc4c4cccnc43)c2)cc1 ; c1ccc(-c2ccc(-n3c4ccccc4c4cccc(-c5ccc6c(c5)c5cccnc5n6-c5cccc(-c6ccccc6)c5)c43)cc2)cc1 ; c1ccc(-c2ccc(-n3c4ccc(-c5cccc6c7ccccc7n(-c7ccc(-c8ccccc8)cc7)c56)cc4c4cccnc43)cc2)cc1 ; c1ccc(-c2cccc(-n3c4ccc(-c5cccc6c7ccccc7n(-c7cccc(-c8ccccc8)c7)c56)cc4c4cccnc43)c2)cc1]

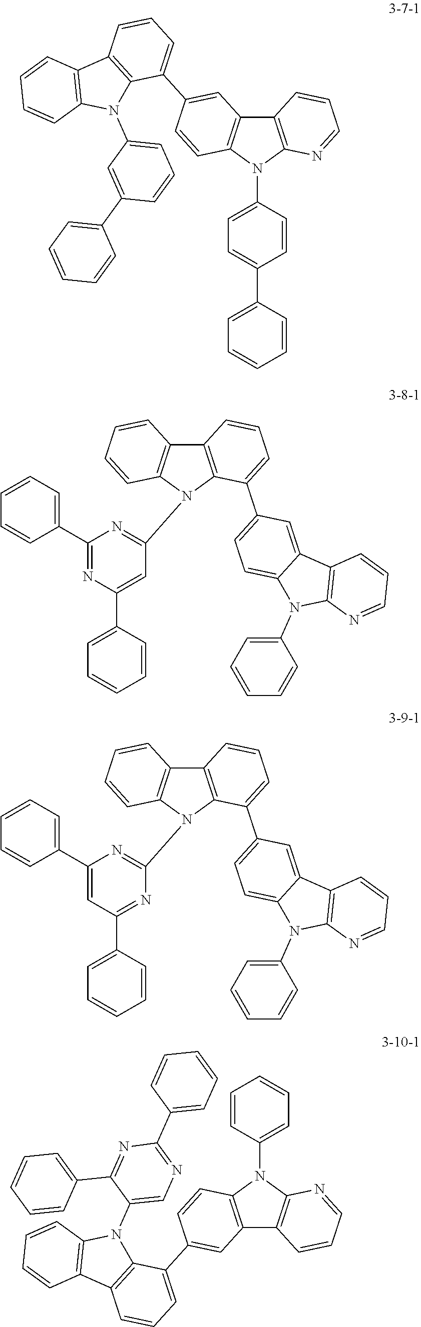 [SMILES: c1ccc(-c2ccc(-n3c4ccc(-c5cccc6c7ccccc7n(-c7cccc(-c8ccccc8)c7)c56)cc4c4cccnc43)cc2)cc1 ; c1ccc(-c2cc(-n3c4ccccc4c4cccc(-c5ccc6c(c5)c5cccnc5n6-c5ccccc5)c43)nc(-c3ccccc3)n2)cc1 ; c1ccc(-c2cc(-c3ccccc3)nc(-n3c4ccccc4c4cccc(-c5ccc6c(c5)c5cccnc5n6-c5ccccc5)c43)n2)cc1 ; c1ccc(-c2ncc(-n3c4ccccc4c4cccc(-c5ccc6c(c5)c5cccnc5n6-c5ccccc5)c43)c(-c3ccccc3)n2)cc1]

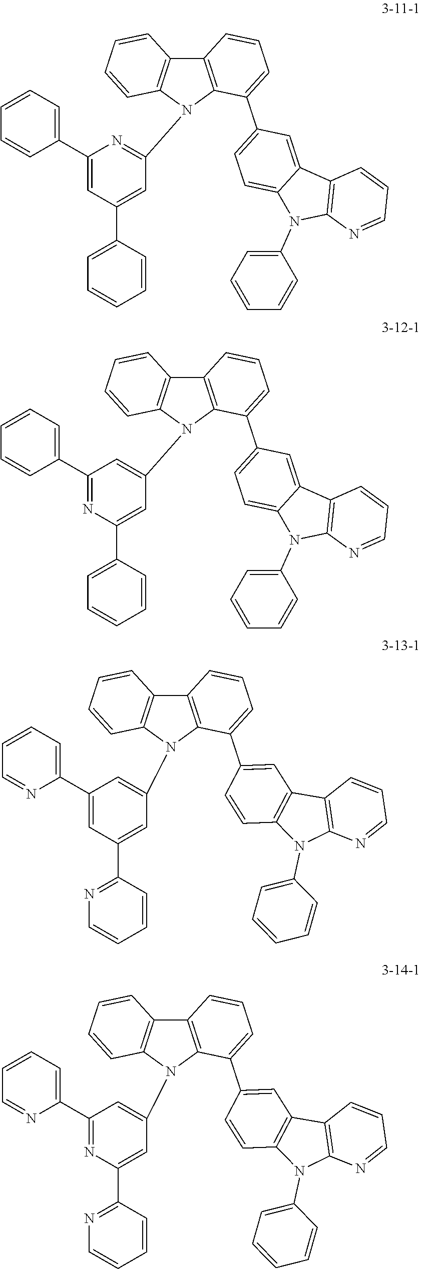 [SMILES: c1ccc(-c2cc(-c3ccccc3)nc(-n3c4ccccc4c4cccc(-c5ccc6c(c5)c5cccnc5n6-c5ccccc5)c43)c2)cc1 ; c1ccc(-c2cc(-n3c4ccccc4c4cccc(-c5ccc6c(c5)c5cccnc5n6-c5ccccc5)c43)cc(-c3ccccc3)n2)cc1 ; c1ccc(-n2c3ccc(-c4cccc5c6ccccc6n(-c6cc(-c7ccccn7)cc(-c7ccccn7)c6)c45)cc3c3cccnc32)cc1 ; c1ccc(-n2c3ccc(-c4cccc5c6ccccc6n(-c6cc(-c7ccccn7)nc(-c7ccccn7)c6)c45)cc3c3cccnc32)cc1]

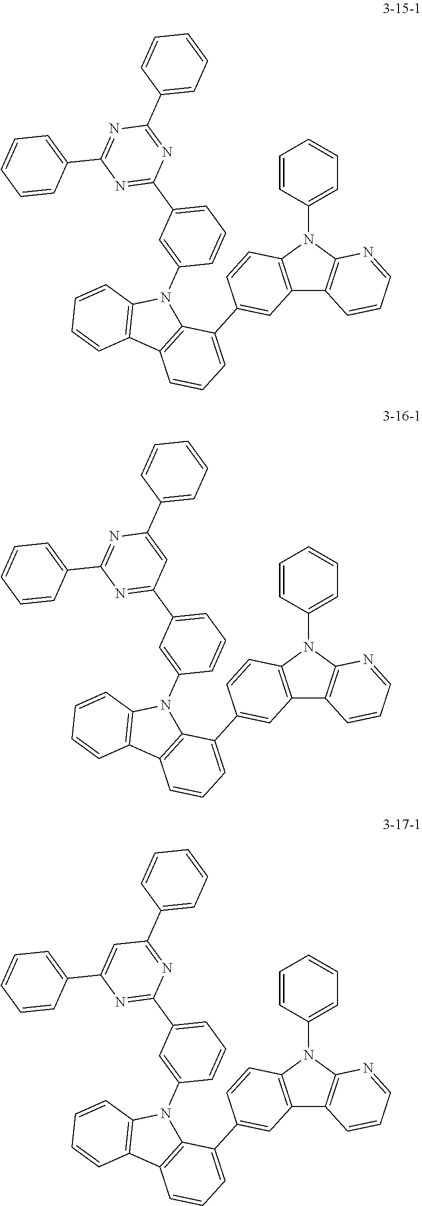 [SMILES: c1ccc(-c2nc(-c3ccccc3)nc(-c3cccc(-n4c5ccccc5c5cccc(-c6ccc7c(c6)c6cccnc6n7-c6ccccc6)c54)c3)n2)cc1 ; c1ccc(-c2cc(-c3cccc(-n4c5ccccc5c5cccc(-c6ccc7c(c6)c6cccnc6n7-c6ccccc6)c54)c3)nc(-c3ccccc3)n2)cc1 ; c1ccc(-c2cc(-c3ccccc3)nc(-c3cccc(-n4c5ccccc5c5cccc(-c6ccc7c(c6)c6cccnc6n7-c6ccccc6)c54)c3)n2)cc1]

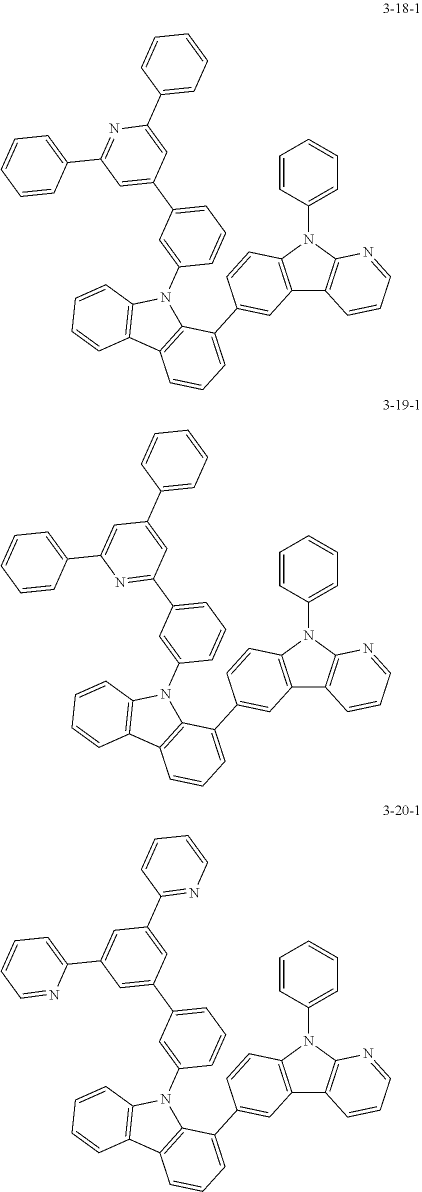

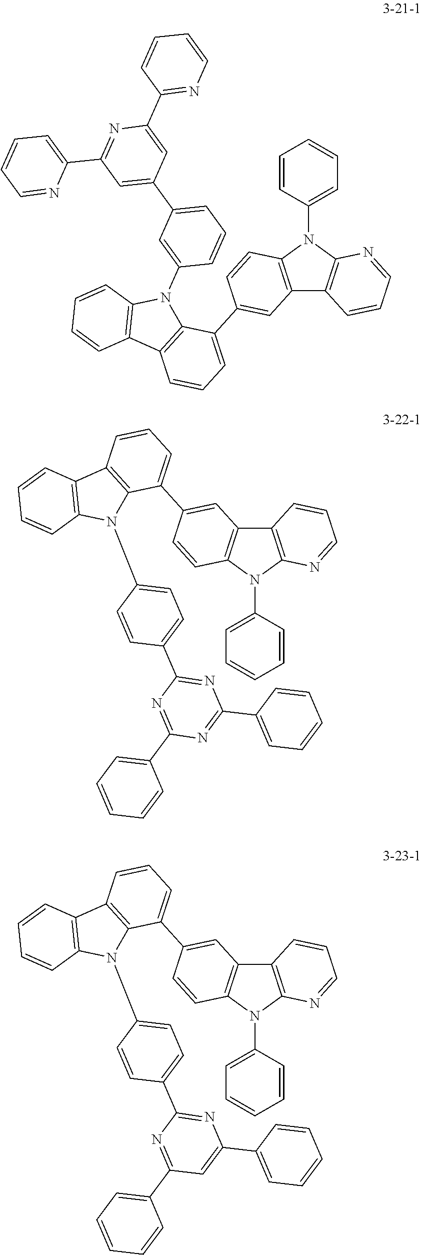 [SMILES: c1ccc(-n2c3ccc(-c4cccc5c6ccccc6n(-c6cccc(-c7cc(-c8ccccn8)nc(-c8ccccn8)c7)c6)c45)cc3c3cccnc32)cc1 ; c1ccc(-c2nc(-c3ccccc3)nc(-c3ccc(-n4c5ccccc5c5cccc(-c6ccc7c(c6)c6cccnc6n7-c6ccccc6)c54)cc3)n2)cc1 ; c1ccc(-c2cc(-c3ccccc3)nc(-c3ccc(-n4c5ccccc5c5cccc(-c6ccc7c(c6)c6cccnc6n7-c6ccccc6)c54)cc3)n2)cc1]

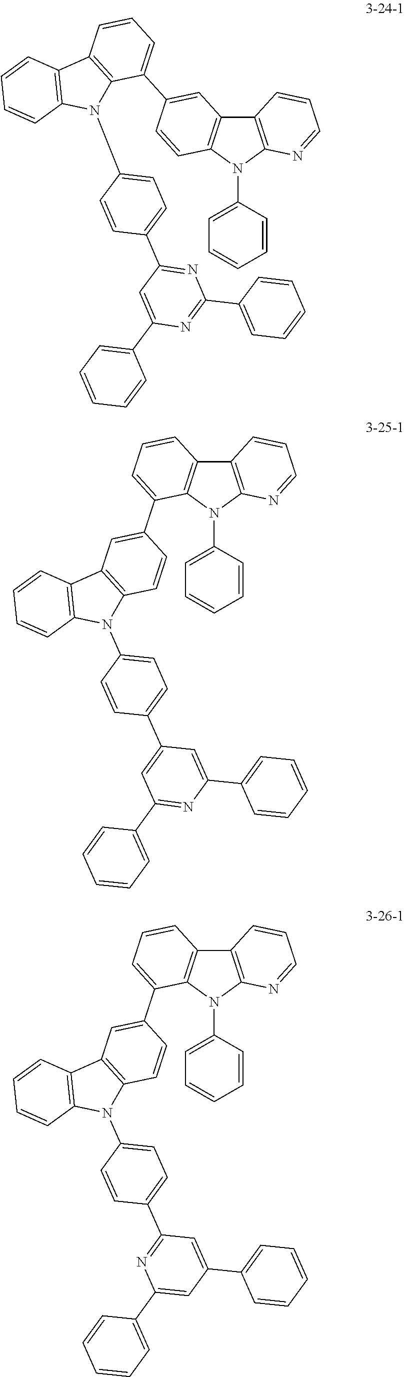

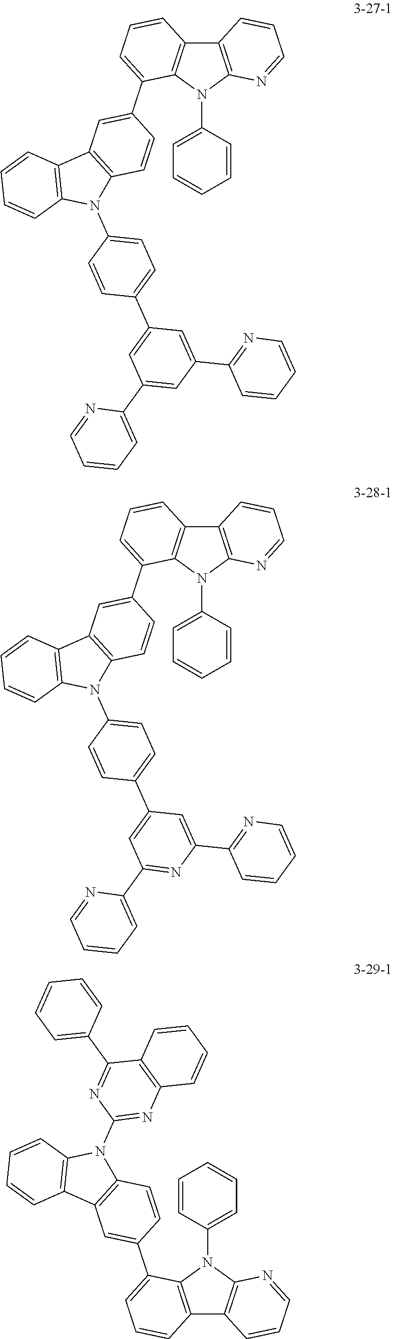

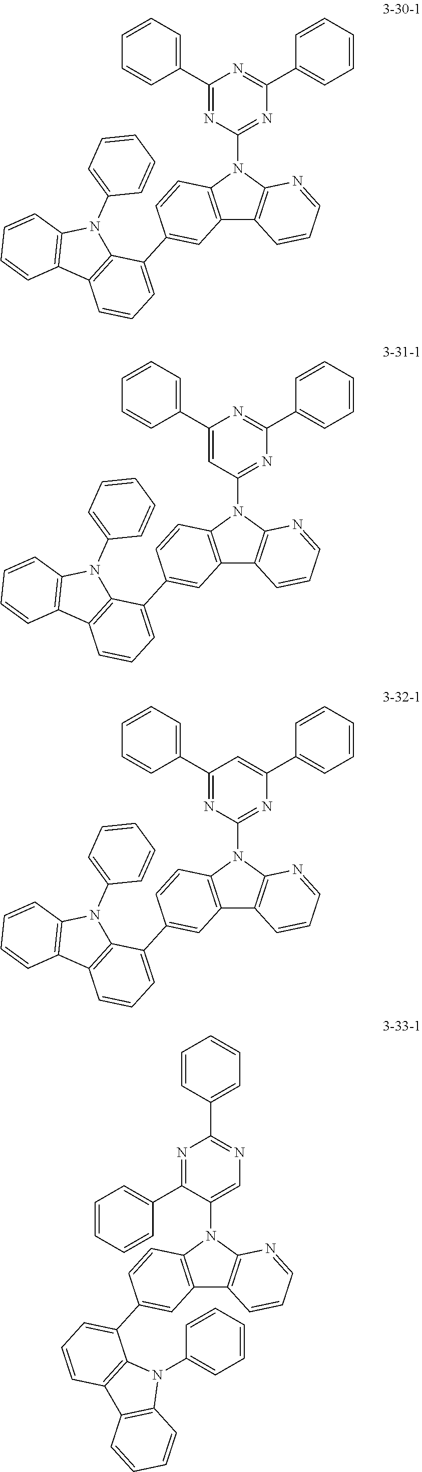 [SMILES: c1ccc(-c2nc(-c3ccccc3)nc(-n3c4ccc(-c5cccc6c7ccccc7n(-c7ccccc7)c56)cc4c4cccnc43)n2)cc1 ; c1ccc(-c2cc(-n3c4ccc(-c5cccc6c7ccccc7n(-c7ccccc7)c56)cc4c4cccnc43)nc(-c3ccccc3)n2)cc1 ; c1ccc(-c2cc(-c3ccccc3)nc(-n3c4ccc(-c5cccc6c7ccccc7n(-c7ccccc7)c56)cc4c4cccnc43)n2)cc1 ; c1ccc(-c2ncc(-n3c4ccc(-c5cccc6c7ccccc7n(-c7ccccc7)c56)cc4c4cccnc43)c(-c3ccccc3)n2)cc1]

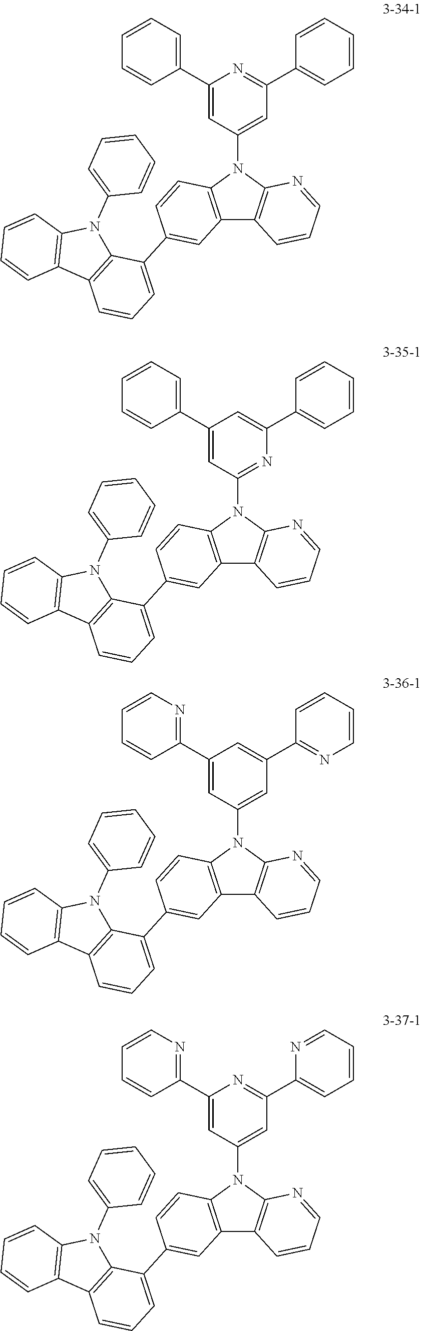

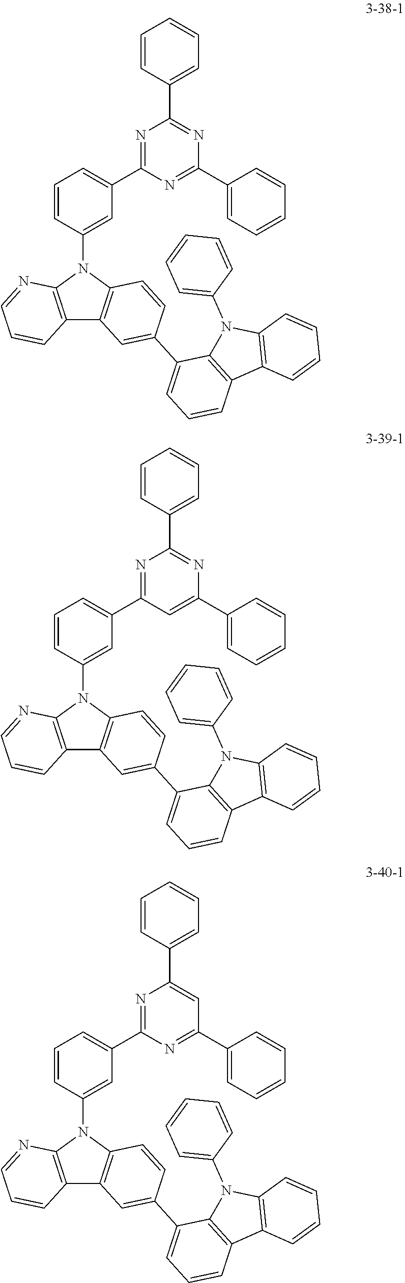

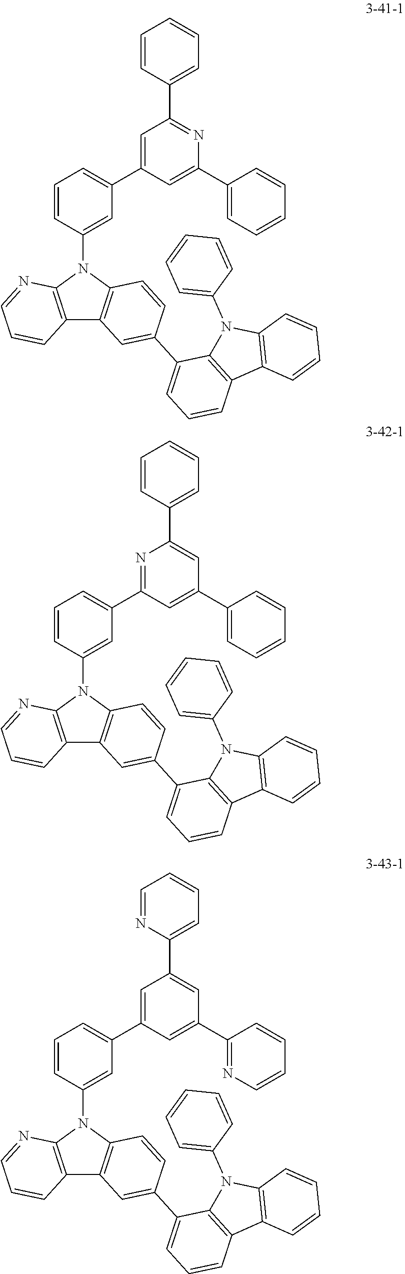

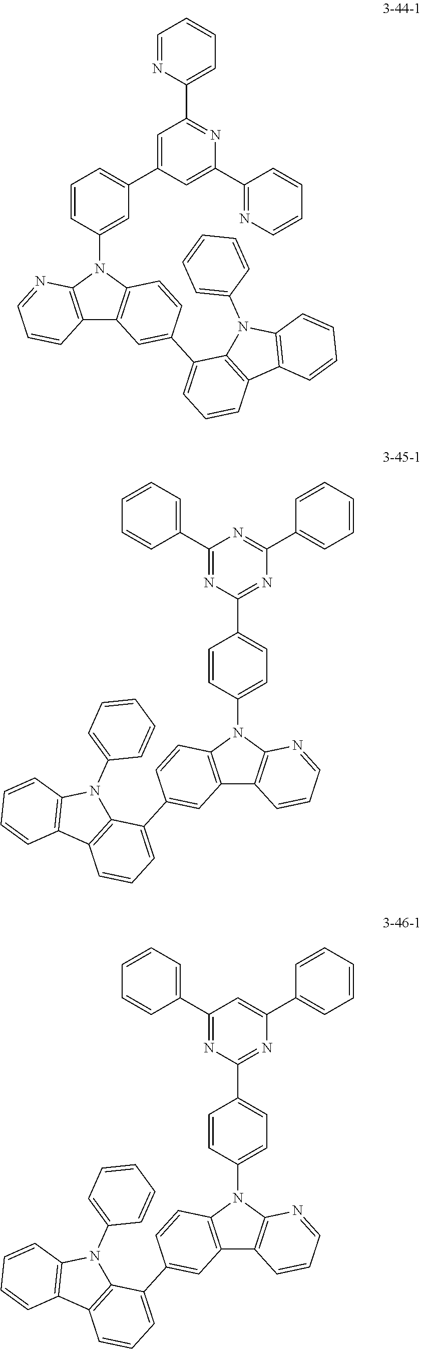 [SMILES: c1ccc(-n2c3ccccc3c3cccc(-c4ccc5c(c4)c4cccnc4n5-c4cccc(-c5cc(-c6ccccn6)nc(-c6ccccn6)c5)c4)c32)cc1 ; c1ccc(-c2nc(-c3ccccc3)nc(-c3ccc(-n4c5ccc(-c6cccc7c8ccccc8n(-c8ccccc8)c67)cc5c5cccnc54)cc3)n2)cc1 ; c1ccc(-c2cc(-c3ccccc3)nc(-c3ccc(-n4c5ccc(-c6cccc7c8ccccc8n(-c8ccccc8)c67)cc5c5cccnc54)cc3)n2)cc1]

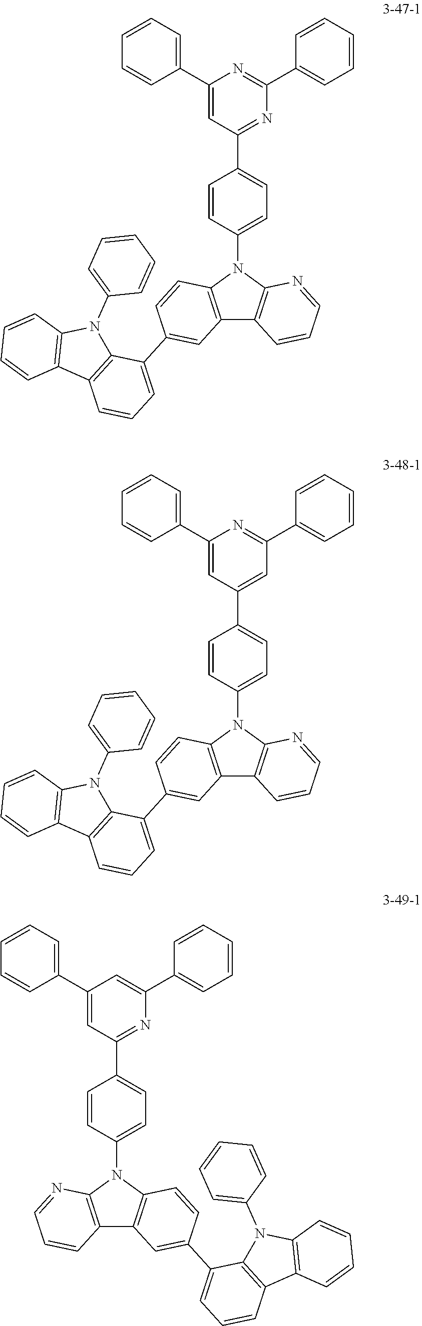 [SMILES: c1ccc(-c2cc(-c3ccc(-n4c5ccc(-c6cccc7c8ccccc8n(-c8ccccc8)c67)cc5c5cccnc54)cc3)nc(-c3ccccc3)n2)cc1 ; c1ccc(-c2cc(-c3ccc(-n4c5ccc(-c6cccc7c8ccccc8n(-c8ccccc8)c67)cc5c5cccnc54)cc3)cc(-c3ccccc3)n2)cc1 ; c1ccc(-c2cc(-c3ccccc3)nc(-c3ccc(-n4c5ccc(-c6cccc7c8ccccc8n(-c8ccccc8)c67)cc5c5cccnc54)cc3)c2)cc1]

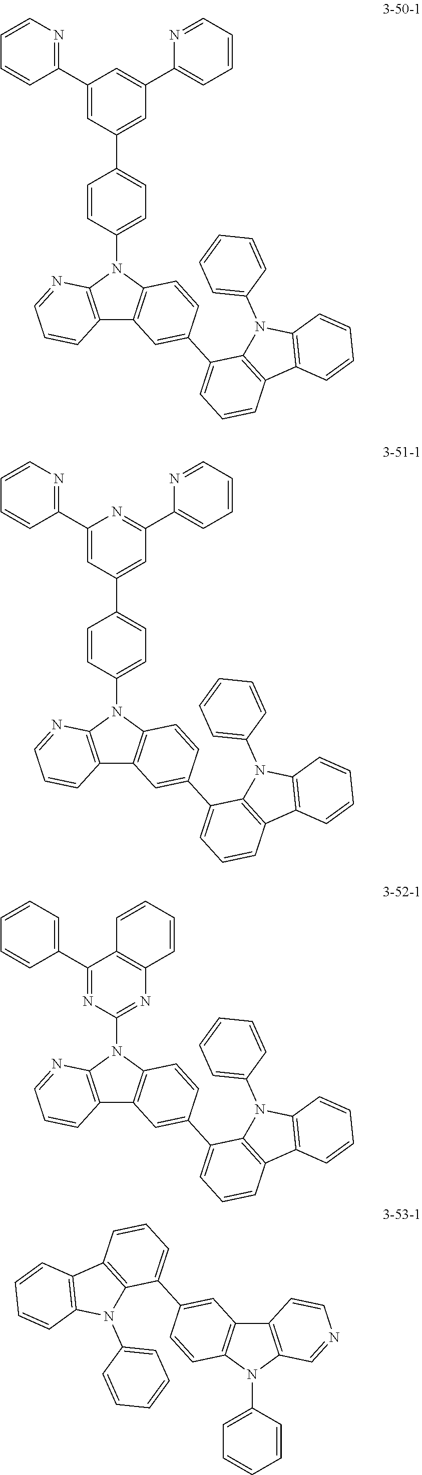 [SMILES: c1ccc(-n2c3ccccc3c3cccc(-c4ccc5c(c4)c4cccnc4n5-c4ccc(-c5cc(-c6ccccn6)cc(-c6ccccn6)c5)cc4)c32)cc1 ; c1ccc(-n2c3ccccc3c3cccc(-c4ccc5c(c4)c4cccnc4n5-c4ccc(-c5cc(-c6ccccn6)nc(-c6ccccn6)c5)cc4)c32)cc1 ; c1ccc(-c2nc(-n3c4ccc(-c5cccc6c7ccccc7n(-c7ccccc7)c56)cc4c4cccnc43)nc3ccccc23)cc1 ; c1ccc(-n2c3ccc(-c4cccc5c6ccccc6n(-c6ccccc6)c45)cc3c3ccncc32)cc1]

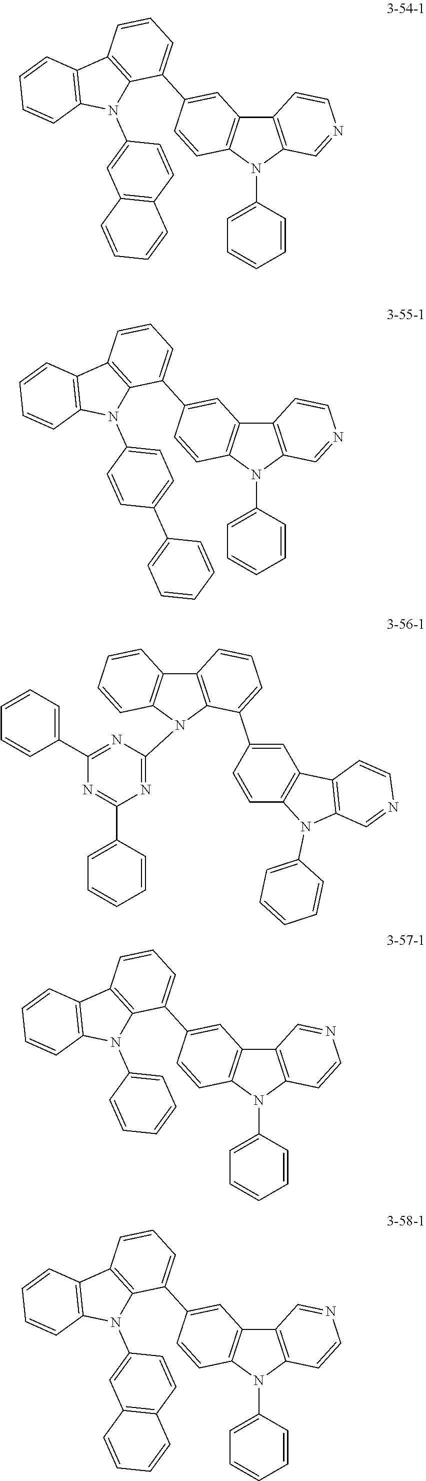 [SMILES: c1ccc(-n2c3ccc(-c4cccc5c6ccccc6n(-c6ccc7ccccc7c6)c45)cc3c3ccncc32)cc1 ; c1ccc(-c2ccc(-n3c4ccccc4c4cccc(-c5ccc6c(c5)c5ccncc5n6-c5ccccc5)c43)cc2)cc1 ; c1ccc(-c2nc(-c3ccccc3)nc(-n3c4ccccc4c4cccc(-c5ccc6c(c5)c5ccncc5n6-c5ccccc5)c43)n2)cc1 ; c1ccc(-n2c3ccncc3c3cc(-c4cccc5c6ccccc6n(-c6ccccc6)c45)ccc32)cc1 ; c1ccc(-n2c3ccncc3c3cc(-c4cccc5c6ccccc6n(-c6ccc7ccccc7c6)c45)ccc32)cc1]

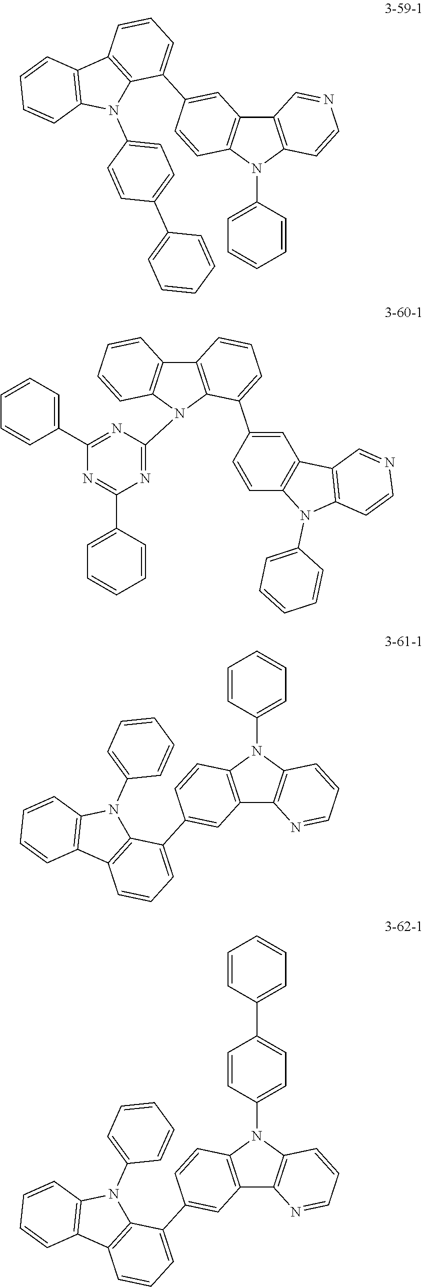 [SMILES: c1ccc(-c2ccc(-n3c4ccccc4c4cccc(-c5ccc6c(c5)c5cnccc5n6-c5ccccc5)c43)cc2)cc1 ; c1ccc(-c2nc(-c3ccccc3)nc(-n3c4ccccc4c4cccc(-c5ccc6c(c5)c5cnccc5n6-c5ccccc5)c43)n2)cc1 ; c1ccc(-n2c3ccc(-c4cccc5c6ccccc6n(-c6ccccc6)c45)cc3c3ncccc32)cc1 ; c1ccc(-c2ccc(-n3c4ccc(-c5cccc6c7ccccc7n(-c7ccccc7)c56)cc4c4ncccc43)cc2)cc1]

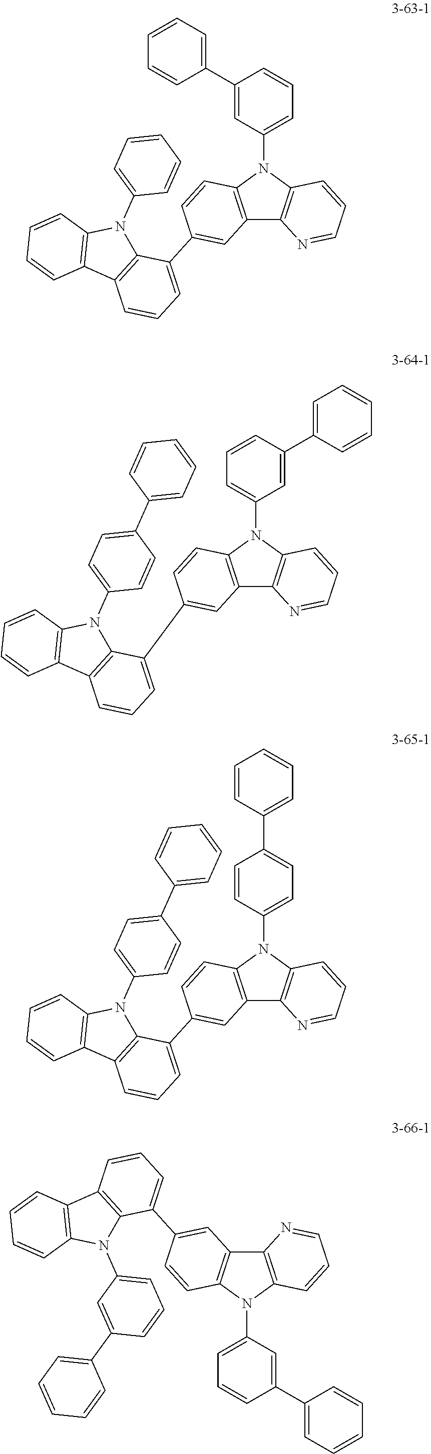 [SMILES: c1ccc(-c2cccc(-n3c4ccc(-c5cccc6c7ccccc7n(-c7ccccc7)c56)cc4c4ncccc43)c2)cc1 ; c1ccc(-c2ccc(-n3c4ccccc4c4cccc(-c5ccc6c(c5)c5ncccc5n6-c5cccc(-c6ccccc6)c5)c43)cc2)cc1 ; c1ccc(-c2ccc(-n3c4ccc(-c5cccc6c7ccccc7n(-c7ccc(-c8ccccc8)cc7)c56)cc4c4ncccc43)cc2)cc1 ; c1ccc(-c2cccc(-n3c4ccc(-c5cccc6c7ccccc7n(-c7cccc(-c8ccccc8)c7)c56)cc4c4ncccc43)c2)cc1]

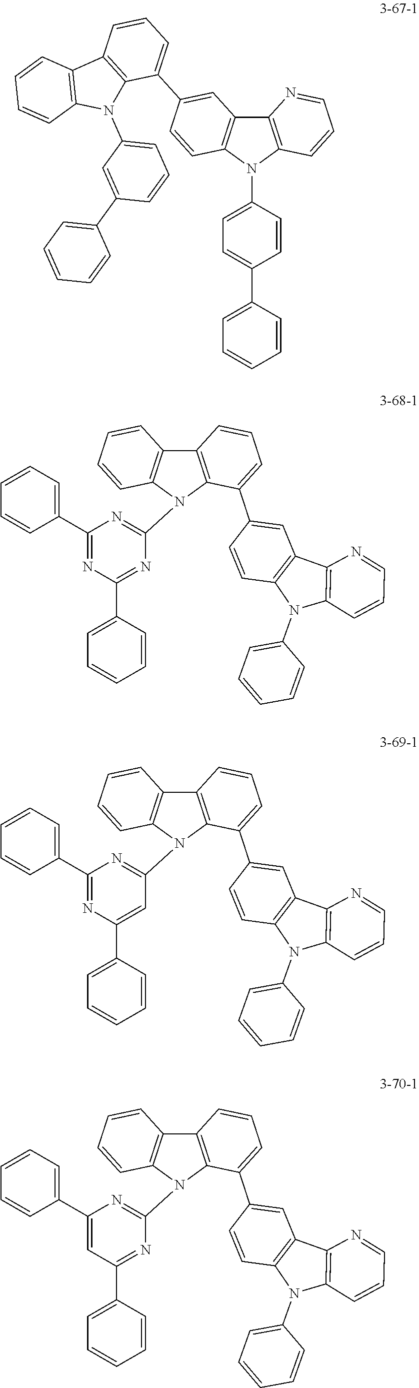 [SMILES: c1ccc(-c2ccc(-n3c4ccc(-c5cccc6c7ccccc7n(-c7cccc(-c8ccccc8)c7)c56)cc4c4ncccc43)cc2)cc1 ; c1ccc(-c2nc(-c3ccccc3)nc(-n3c4ccccc4c4cccc(-c5ccc6c(c5)c5ncccc5n6-c5ccccc5)c43)n2)cc1 ; c1ccc(-c2cc(-n3c4ccccc4c4cccc(-c5ccc6c(c5)c5ncccc5n6-c5ccccc5)c43)nc(-c3ccccc3)n2)cc1 ; c1ccc(-c2cc(-c3ccccc3)nc(-n3c4ccccc4c4cccc(-c5ccc6c(c5)c5ncccc5n6-c5ccccc5)c43)n2)cc1]

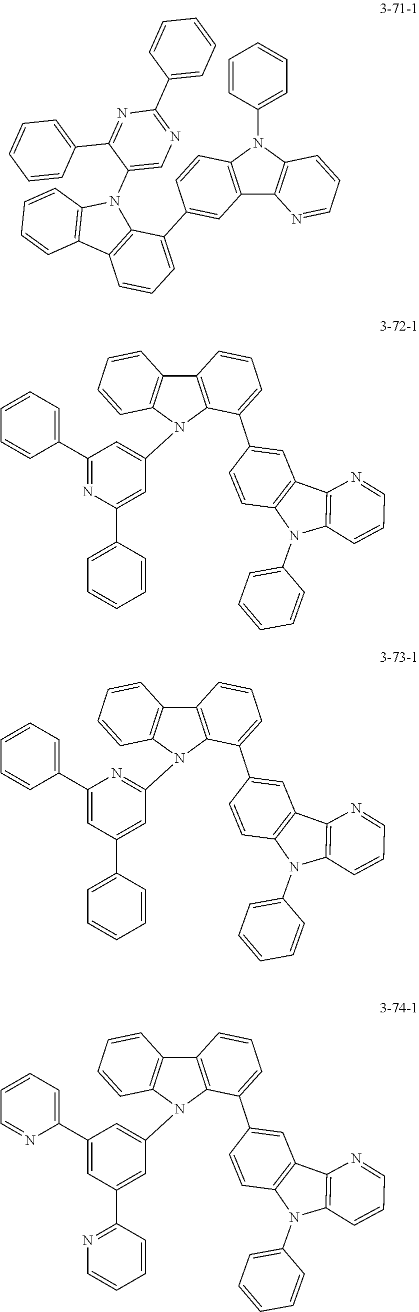 [SMILES: c1ccc(-c2ncc(-n3c4ccccc4c4cccc(-c5ccc6c(c5)c5ncccc5n6-c5ccccc5)c43)c(-c3ccccc3)n2)cc1 ; c1ccc(-c2cc(-n3c4ccccc4c4cccc(-c5ccc6c(c5)c5ncccc5n6-c5ccccc5)c43)cc(-c3ccccc3)n2)cc1 ; c1ccc(-c2cc(-c3ccccc3)nc(-n3c4ccccc4c4cccc(-c5ccc6c(c5)c5ncccc5n6-c5ccccc5)c43)c2)cc1 ; c1ccc(-n2c3ccc(-c4cccc5c6ccccc6n(-c6cc(-c7ccccn7)cc(-c7ccccn7)c6)c45)cc3c3ncccc32)cc1]

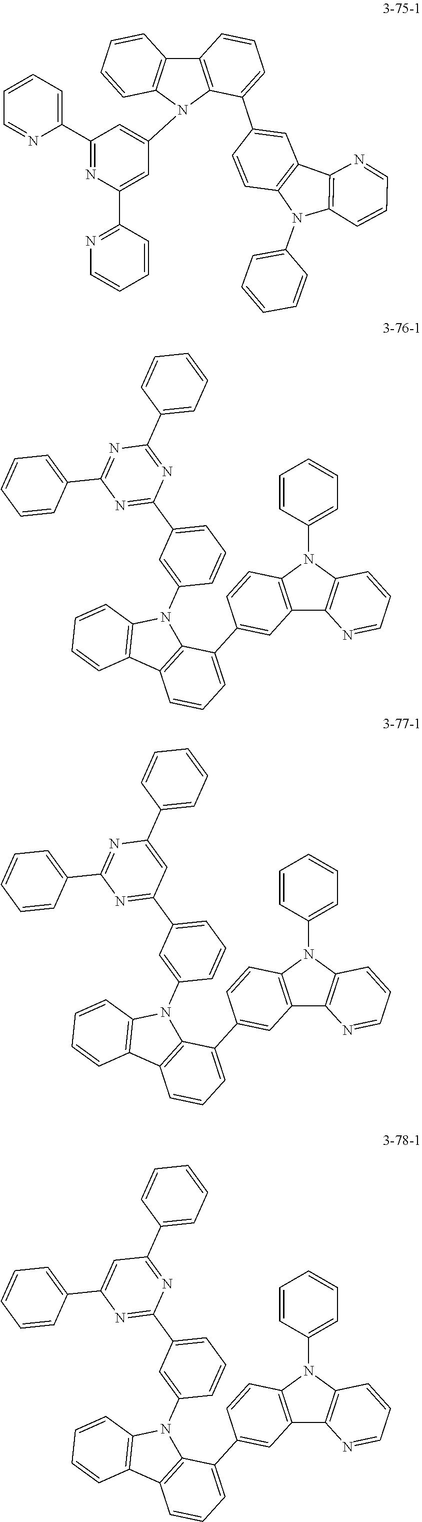

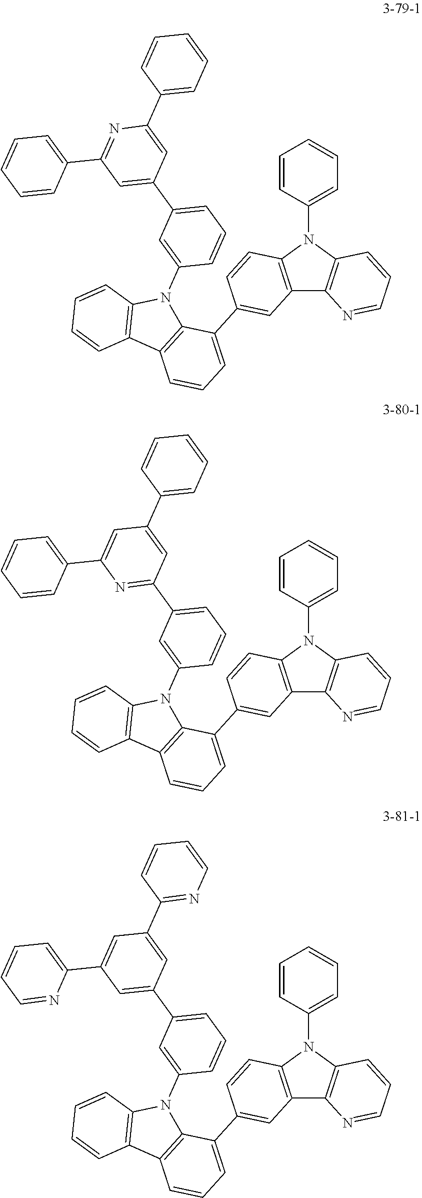 [SMILES: c1ccc(-c2cc(-c3cccc(-n4c5ccccc5c5cccc(-c6ccc7c(c6)c6ncccc6n7-c6ccccc6)c54)c3)cc(-c3ccccc3)n2)cc1 ; c1ccc(-c2cc(-c3ccccc3)nc(-c3cccc(-n4c5ccccc5c5cccc(-c6ccc7c(c6)c6ncccc6n7-c6ccccc6)c54)c3)c2)cc1 ; c1ccc(-n2c3ccc(-c4cccc5c6ccccc6n(-c6cccc(-c7cc(-c8ccccn8)cc(-c8ccccn8)c7)c6)c45)cc3c3ncccc32)cc1]

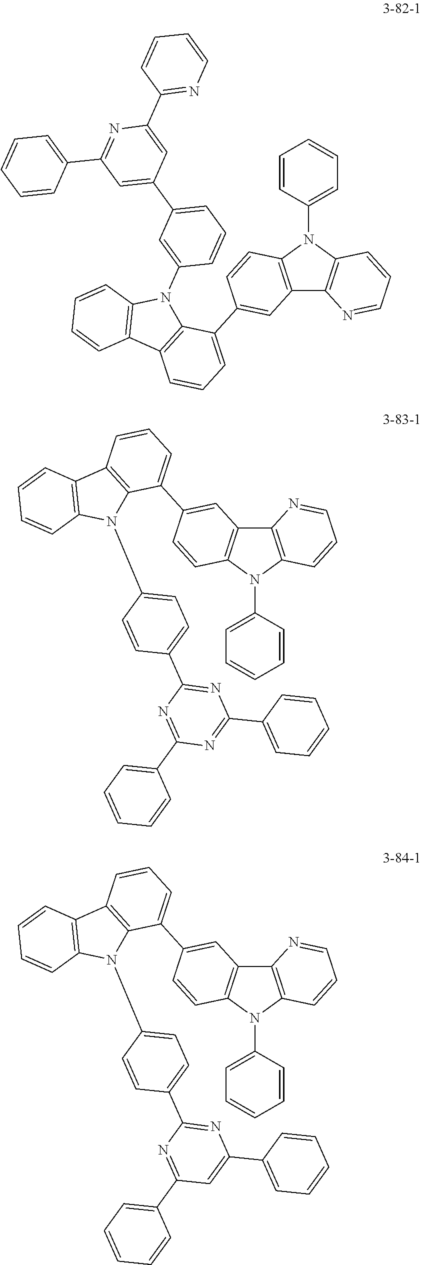

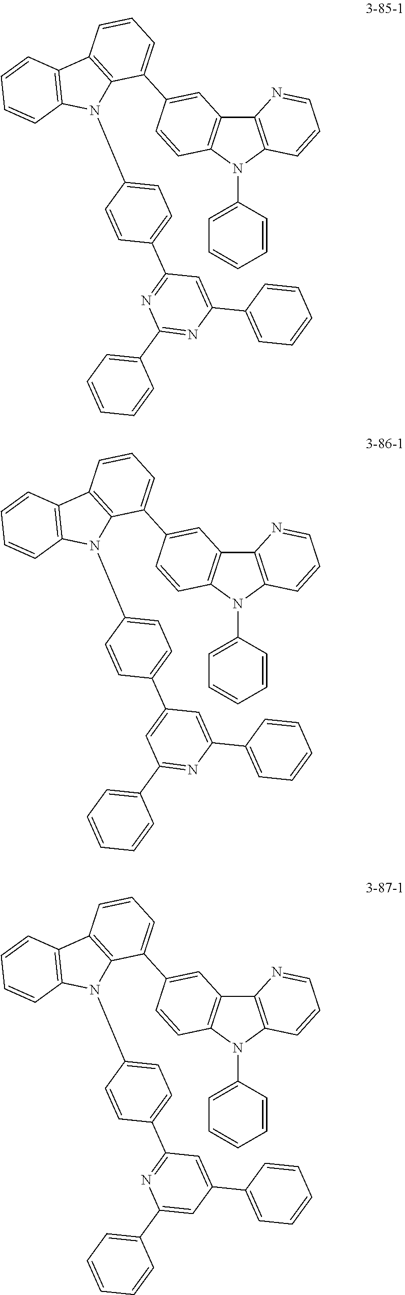

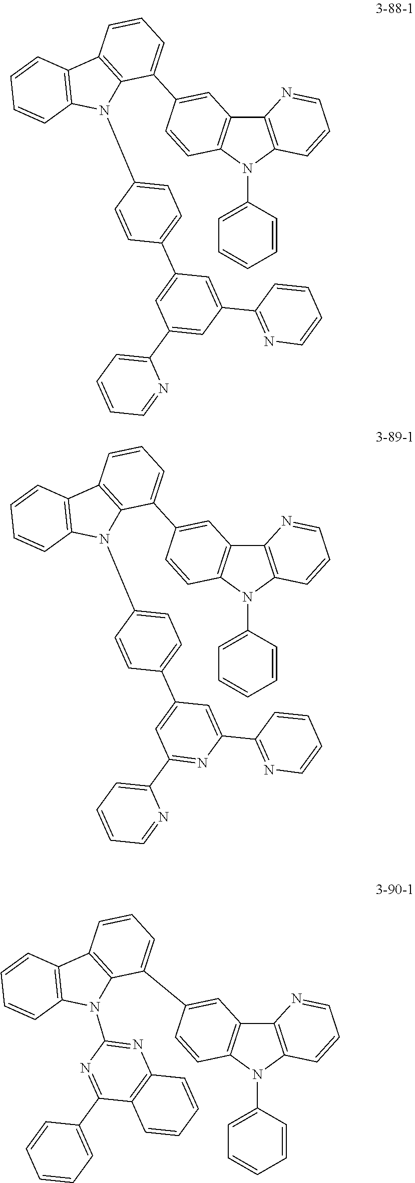

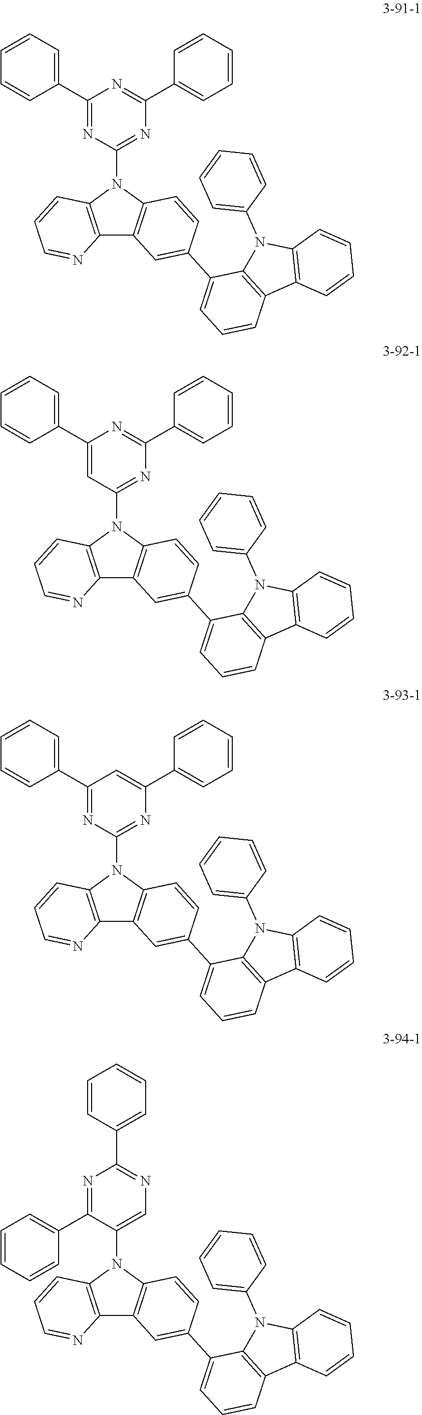

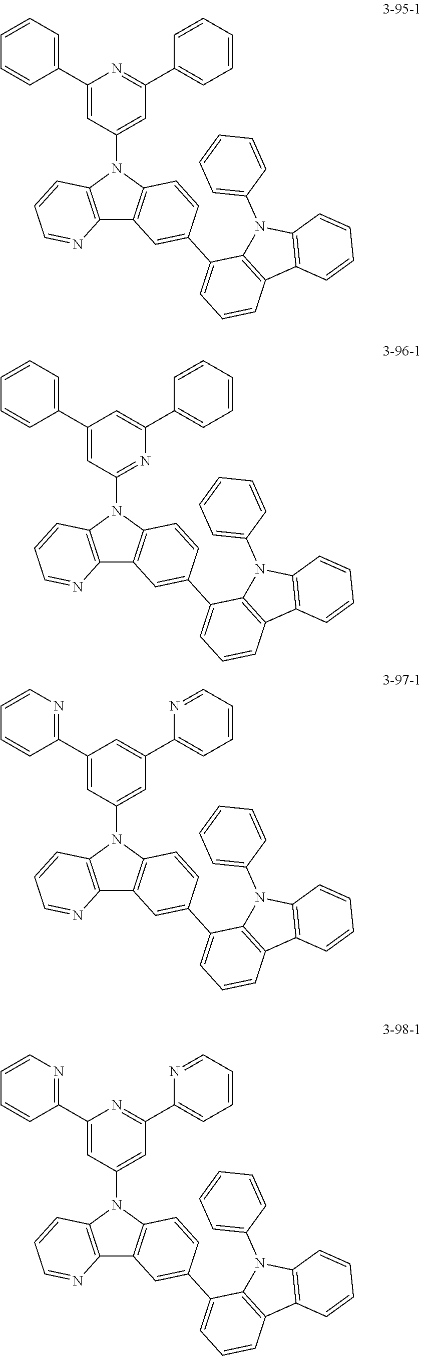

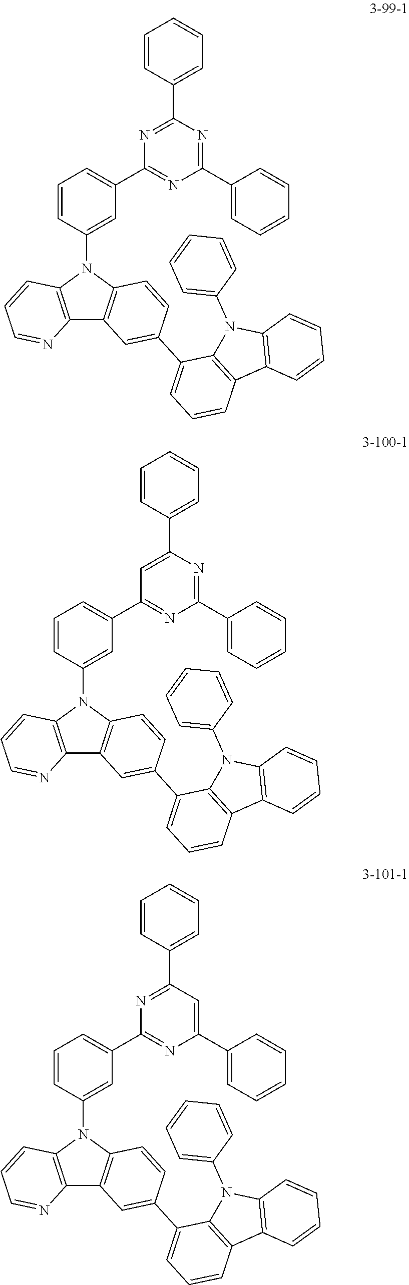

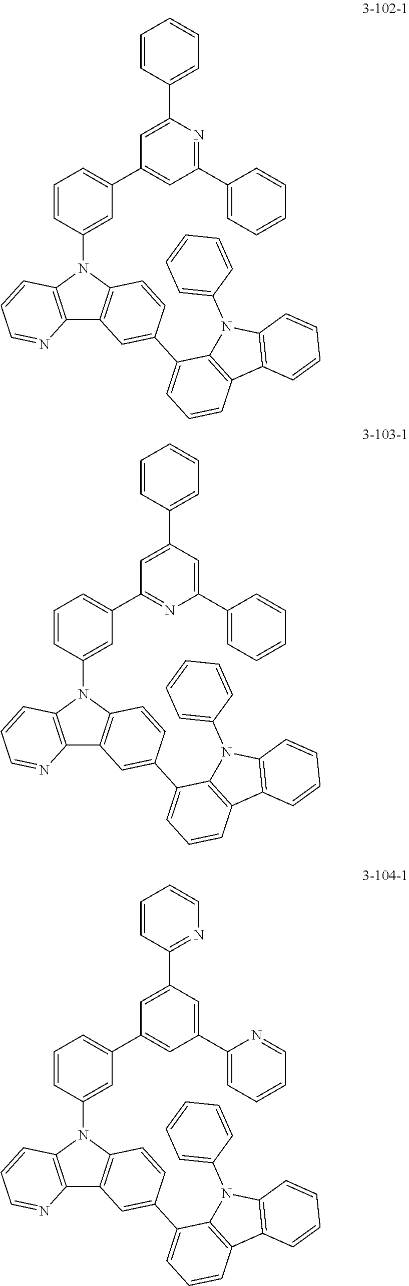

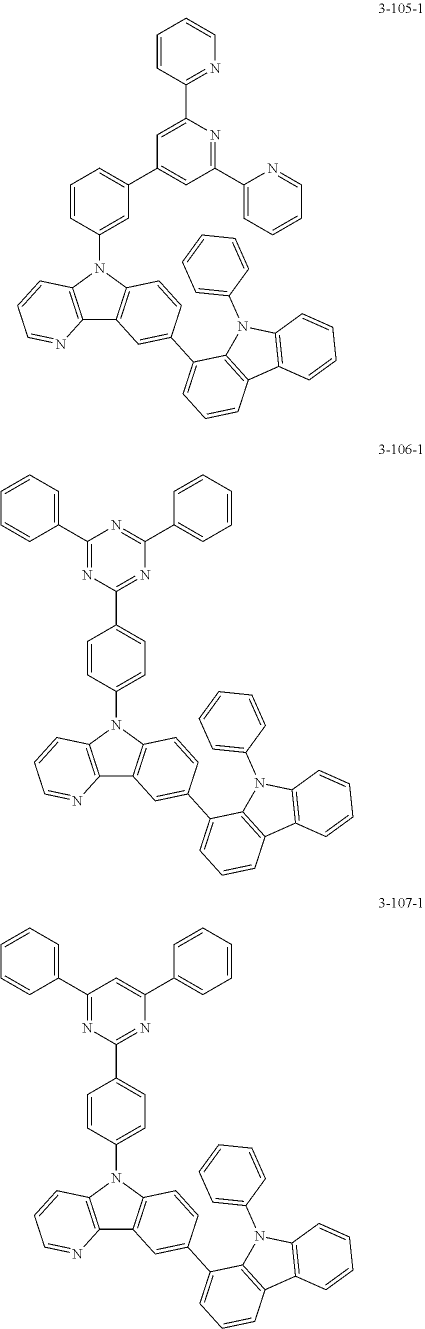 [SMILES: c1ccc(-n2c3ccccc3c3cccc(-c4ccc5c(c4)c4ncccc4n5-c4cccc(-c5cc(-c6ccccn6)nc(-c6ccccn6)c5)c4)c32)cc1 ; c1ccc(-c2nc(-c3ccccc3)nc(-c3ccc(-n4c5ccc(-c6cccc7c8ccccc8n(-c8ccccc8)c67)cc5c5ncccc54)cc3)n2)cc1 ; c1ccc(-c2cc(-c3ccccc3)nc(-c3ccc(-n4c5ccc(-c6cccc7c8ccccc8n(-c8ccccc8)c67)cc5c5ncccc54)cc3)n2)cc1]

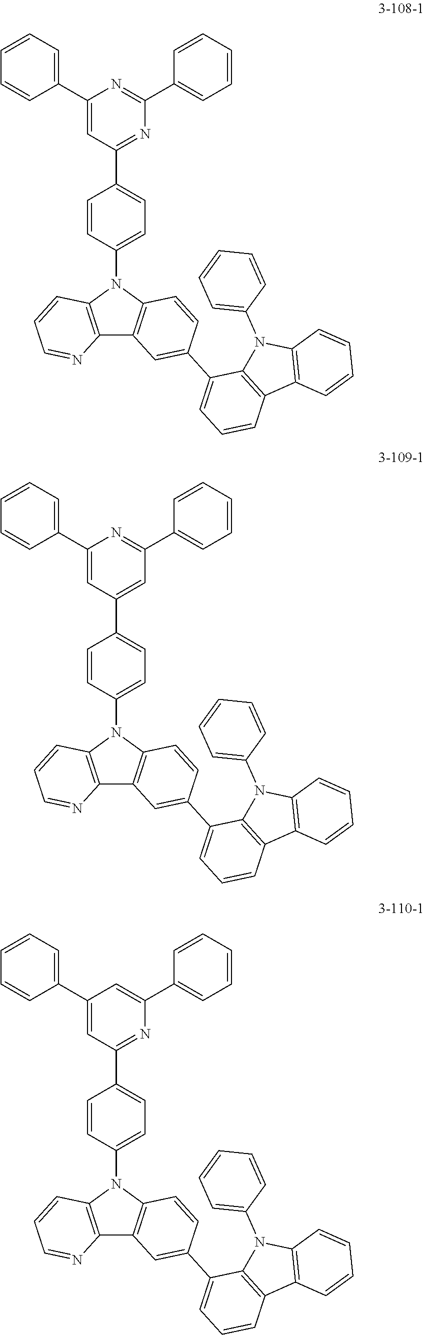 [SMILES: c1ccc(-c2cc(-c3ccc(-n4c5ccc(-c6cccc7c8ccccc8n(-c8ccccc8)c67)cc5c5ncccc54)cc3)nc(-c3ccccc3)n2)cc1 ; c1ccc(-c2cc(-c3ccc(-n4c5ccc(-c6cccc7c8ccccc8n(-c8ccccc8)c67)cc5c5ncccc54)cc3)cc(-c3ccccc3)n2)cc1 ; c1ccc(-c2cc(-c3ccccc3)nc(-c3ccc(-n4c5ccc(-c6cccc7c8ccccc8n(-c8ccccc8)c67)cc5c5ncccc54)cc3)c2)cc1]

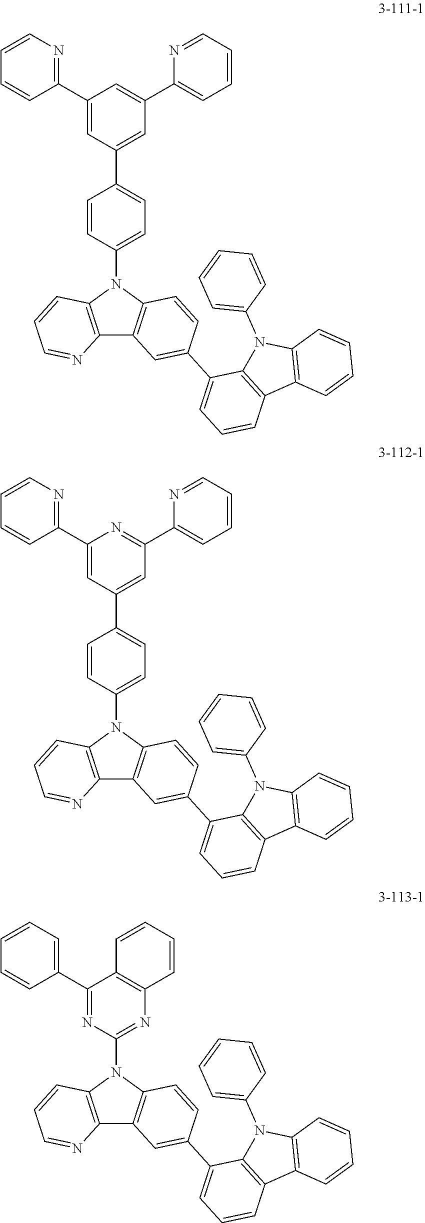 [SMILES: c1ccc(-n2c3ccccc3c3cccc(-c4ccc5c(c4)c4ncccc4n5-c4ccc(-c5cc(-c6ccccn6)cc(-c6ccccn6)c5)cc4)c32)cc1 ; c1ccc(-n2c3ccccc3c3cccc(-c4ccc5c(c4)c4ncccc4n5-c4ccc(-c5cc(-c6ccccn6)nc(-c6ccccn6)c5)cc4)c32)cc1 ; c1ccc(-c2nc(-n3c4ccc(-c5cccc6c7ccccc7n(-c7ccccc7)c56)cc4c4ncccc43)nc3ccccc23)cc1]

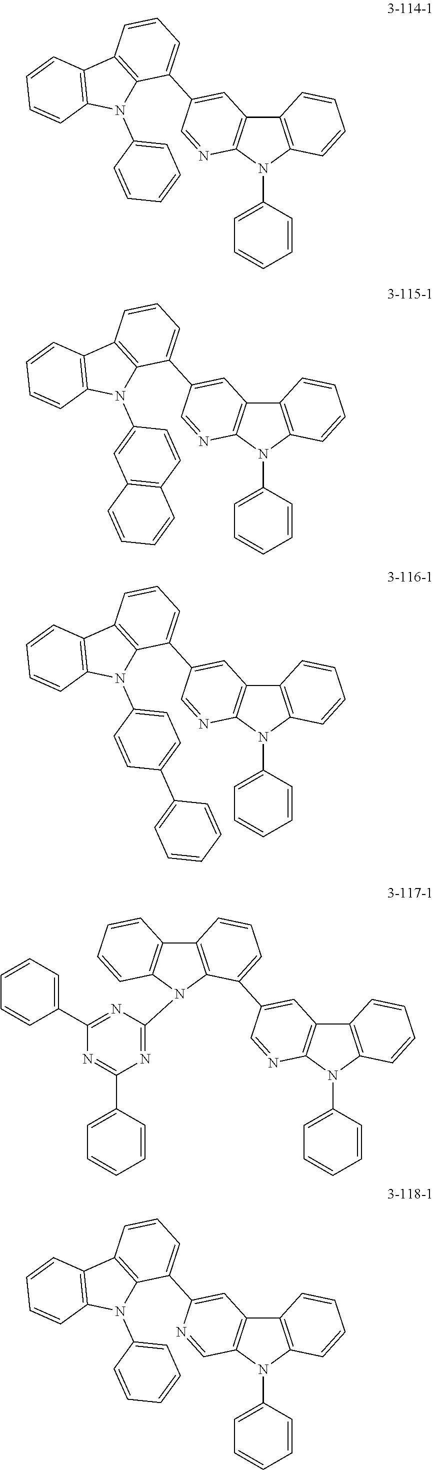

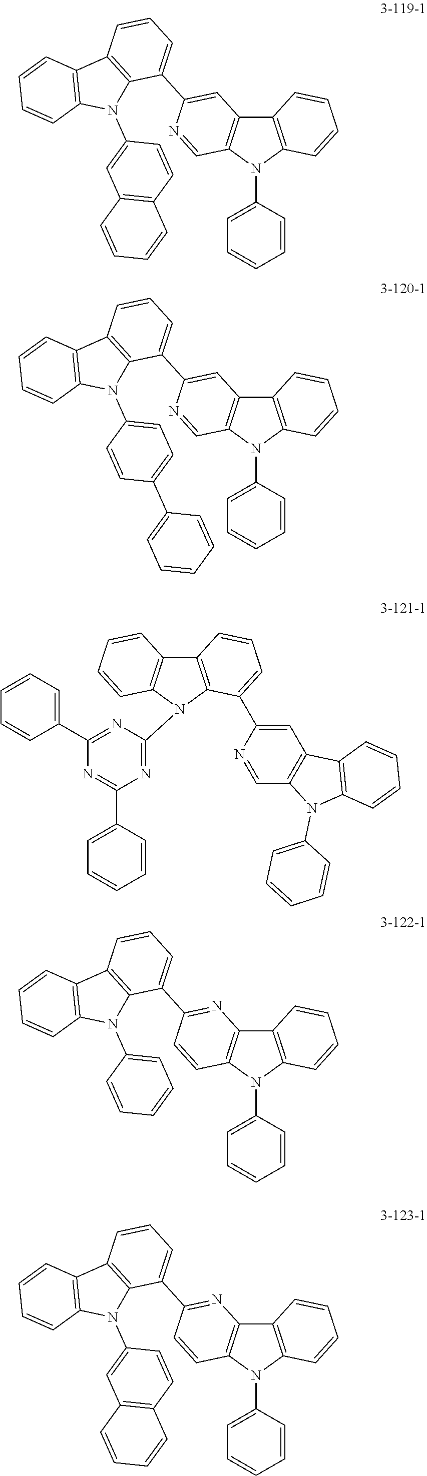

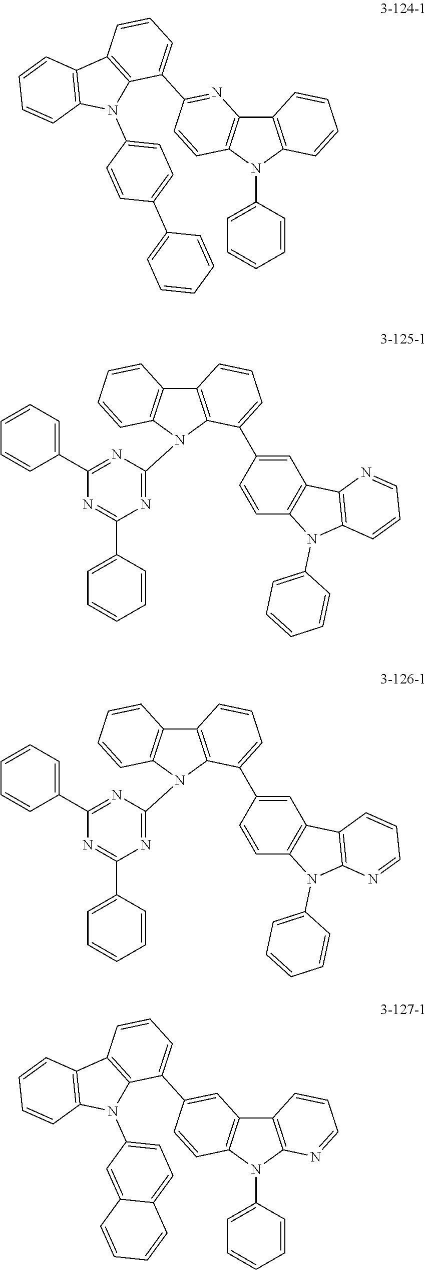 [SMILES: c1ccc(-c2ccc(-n3c4ccccc4c4cccc(-c5ccc6c(n5)c5ccccc5n6-c5ccccc5)c43)cc2)cc1 ; c1ccc(-c2nc(-c3ccccc3)nc(-n3c4ccccc4c4cccc(-c5ccc6c(c5)c5ncccc5n6-c5ccccc5)c43)n2)cc1 ; c1ccc(-c2nc(-c3ccccc3)nc(-n3c4ccccc4c4cccc(-c5ccc6c(c5)c5cccnc5n6-c5ccccc5)c43)n2)cc1 ; c1ccc(-n2c3ccc(-c4cccc5c6ccccc6n(-c6ccc7ccccc7c6)c45)cc3c3cccnc32)cc1]

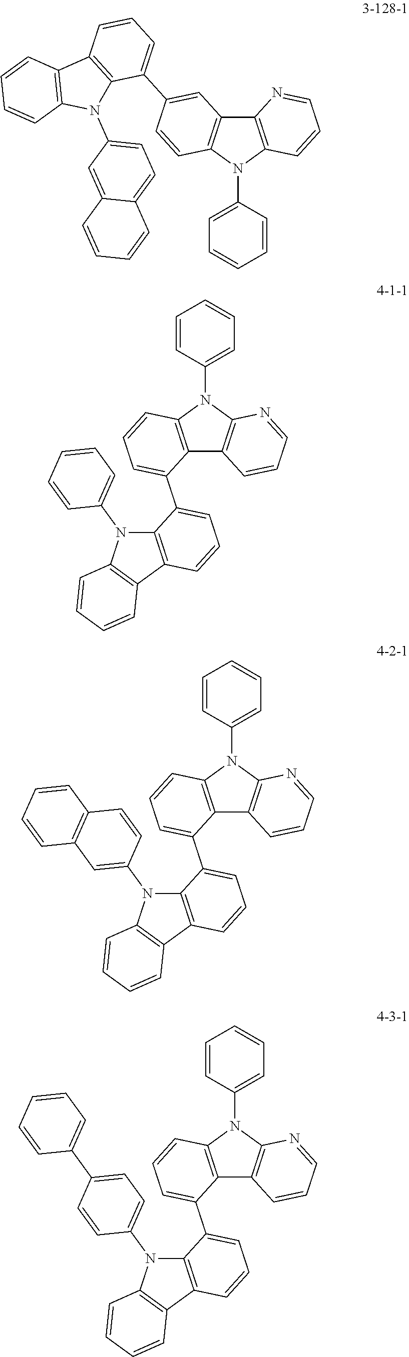

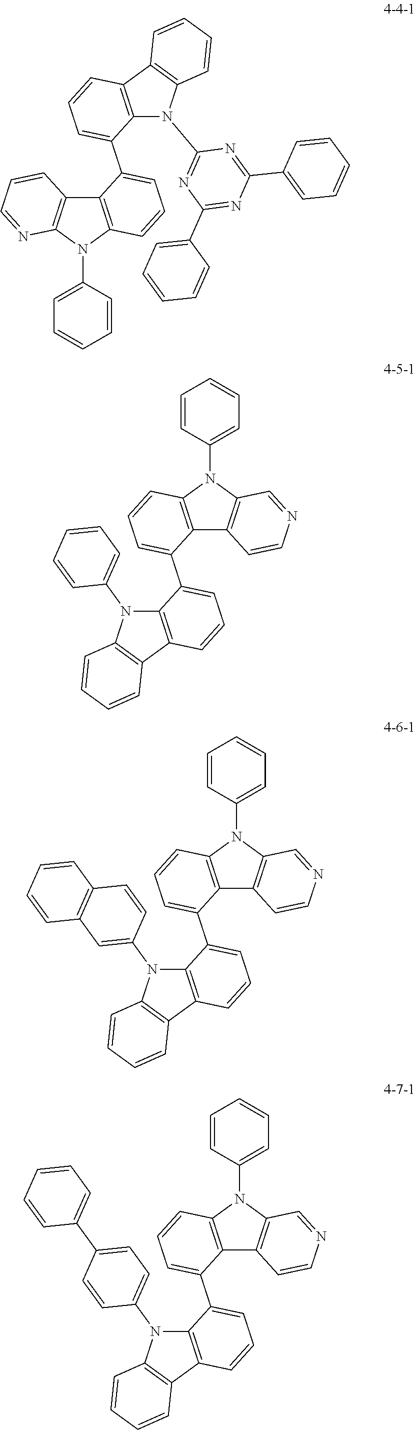 [SMILES: c1ccc(-c2nc(-c3ccccc3)nc(-n3c4ccccc4c4cccc(-c5cccc6c5c5cccnc5n6-c5ccccc5)c43)n2)cc1 ; c1ccc(-n2c3cnccc3c3c(-c4cccc5c6ccccc6n(-c6ccccc6)c45)cccc32)cc1 ; c1ccc(-n2c3cnccc3c3c(-c4cccc5c6ccccc6n(-c6ccc7ccccc7c6)c45)cccc32)cc1 ; c1ccc(-c2ccc(-n3c4ccccc4c4cccc(-c5cccc6c5c5ccncc5n6-c5ccccc5)c43)cc2)cc1]

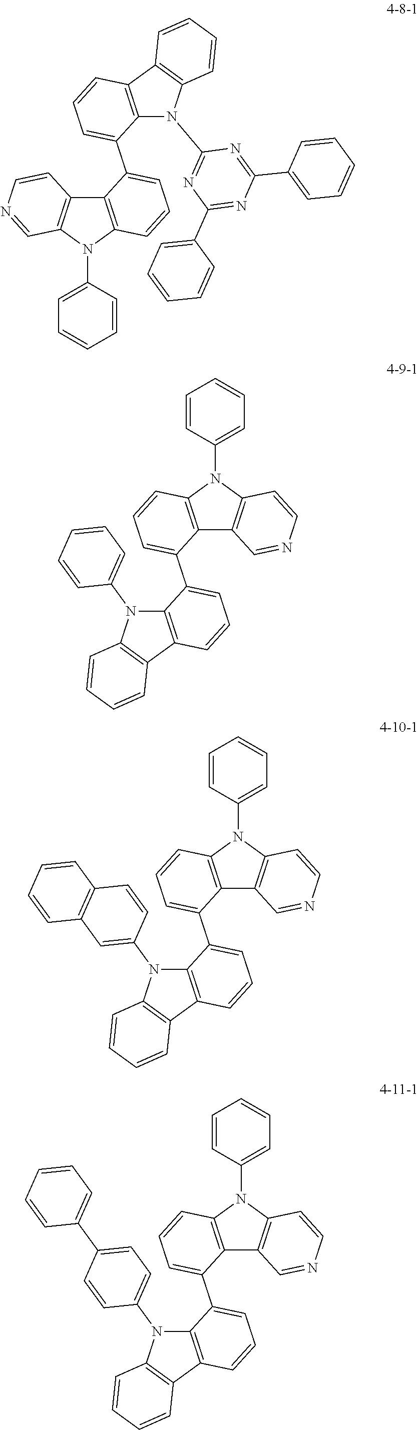 [SMILES: c1ccc(-c2nc(-c3ccccc3)nc(-n3c4ccccc4c4cccc(-c5cccc6c5c5ccncc5n6-c5ccccc5)c43)n2)cc1 ; c1ccc(-n2c3ccncc3c3c(-c4cccc5c6ccccc6n(-c6ccccc6)c45)cccc32)cc1 ; c1ccc(-n2c3ccncc3c3c(-c4cccc5c6ccccc6n(-c6ccc7ccccc7c6)c45)cccc32)cc1 ; c1ccc(-c2ccc(-n3c4ccccc4c4cccc(-c5cccc6c5c5cnccc5n6-c5ccccc5)c43)cc2)cc1]

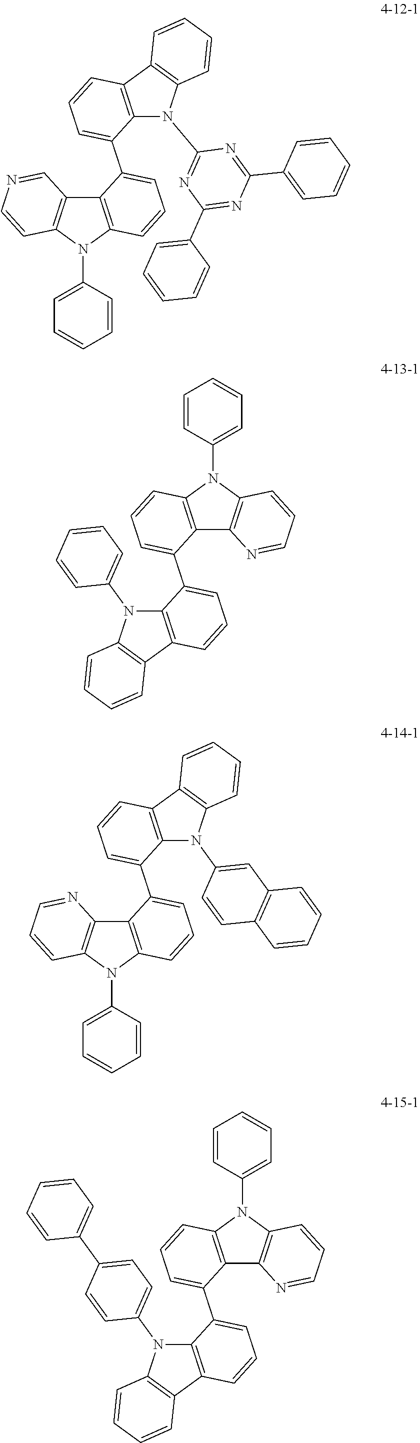 [SMILES: c1ccc(-c2nc(-c3ccccc3)nc(-n3c4ccccc4c4cccc(-c5cccc6c5c5cnccc5n6-c5ccccc5)c43)n2)cc1 ; c1ccc(-n2c3cccnc3c3c(-c4cccc5c6ccccc6n(-c6ccccc6)c45)cccc32)cc1 ; c1ccc(-n2c3cccnc3c3c(-c4cccc5c6ccccc6n(-c6ccc7ccccc7c6)c45)cccc32)cc1 ; c1ccc(-c2ccc(-n3c4ccccc4c4cccc(-c5cccc6c5c5ncccc5n6-c5ccccc5)c43)cc2)cc1]

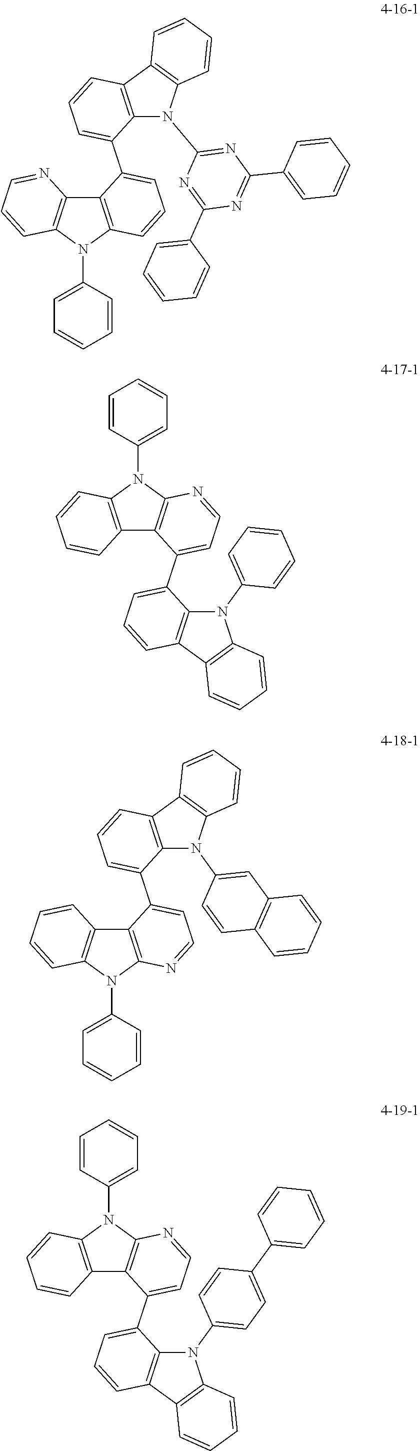

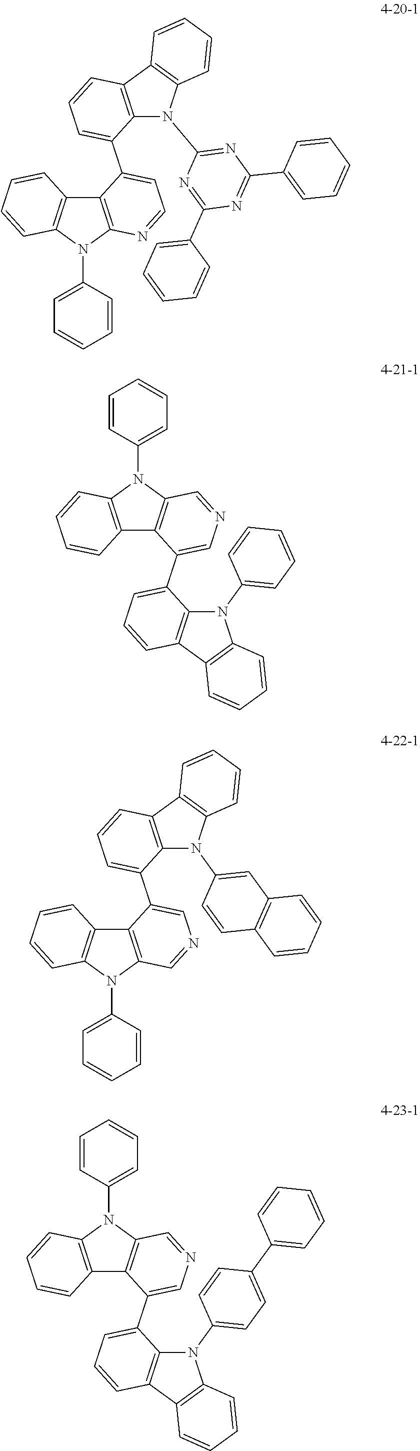

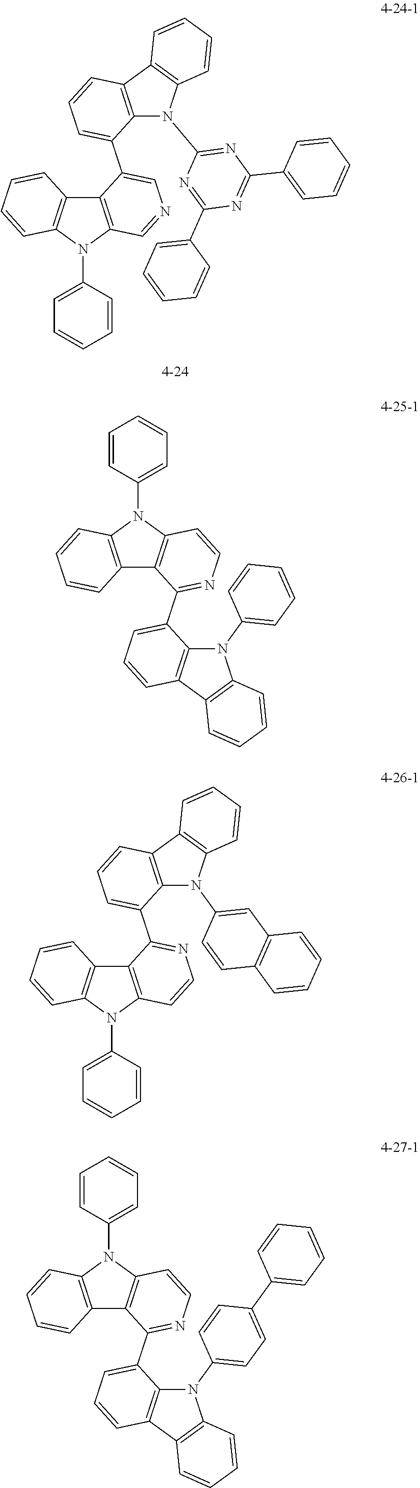

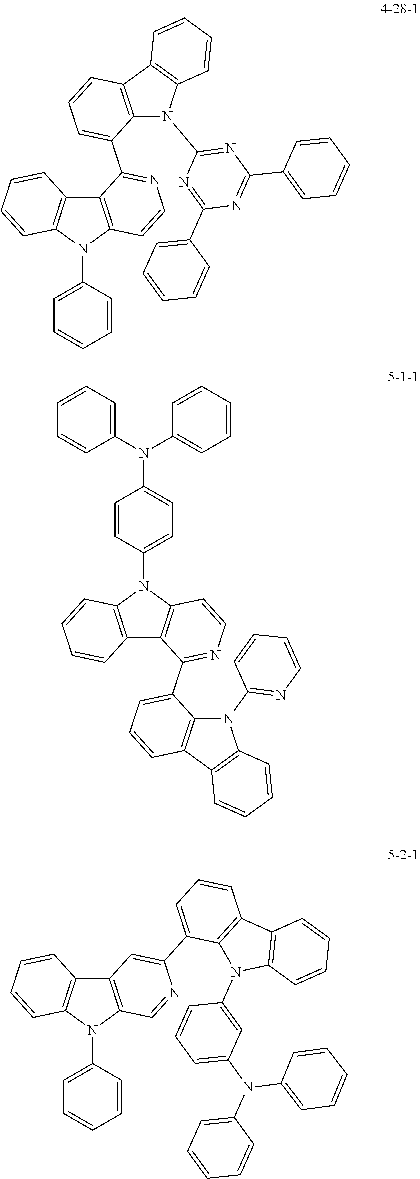

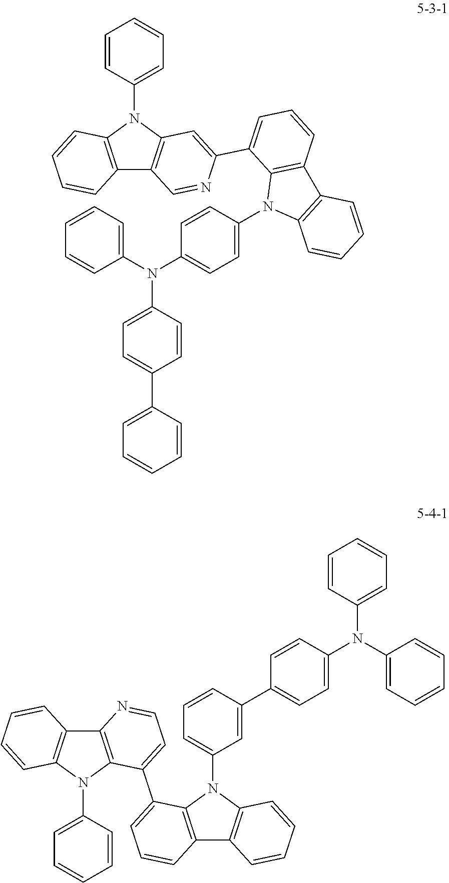 [SMILES: c1ccc(-c2ccc(N(c3ccccc3)c3ccc(-n4c5ccccc5c5cccc(-c6cc7c(cn6)c6ccccc6n7-c6ccccc6)c54)cc3)cc2)cc1 ; c1ccc(N(c2ccccc2)c2ccc(-c3cccc(-n4c5ccccc5c5cccc(-c6ccnc7c8ccccc8n(-c8ccccc8)c67)c54)c3)cc2)cc1]

In another embodiment, the present invention provides a compound for an organic electronic element, represented by Formula 1-1.

In still another embodiment, the present invention provides an organic electronic element containing the compound represented by Formula 1-1.

Here, the organic electronic element may include: a first electrode; a second electrode; and an organic material layer positioned between the first electrode and the second electrode, wherein the organic material layer may contain a compound represented by Formula 1-1, and the compound represented by Formula 1-1 may be contained in at least one of a hole injection layer, a hole transport layer, an auxiliary light emitting layer, a light emitting layer, an electron transport layer, and an electron injection layer for an organic material layer. Especially, the compound represented by Formula 1-1 may be contained in the light emitting layer.

That is, the compound represented by Formula 1-1 may be used as a material for a hole injection layer, a hole transport layer, an auxiliary light emitting layer, a light emitting layer, an electron transport layer, or an electron injection layer. Especially, the compound represented by Formula 1-1 may be used as a material for the light emitting layer. The present invention provides, specifically, an organic electronic element including the organic material layer containing one of the compounds represented by Formulas 1-2 to 1-9, and more specifically, an organic electronic element including the organic material layer containing the compound represented by an individual formula (1-1-1 to 1-28-1, 2-1-1 to 2-128-1, 3-1-1 to 3-128-1, 4-1-1 to 4-28-1, and 5-1-1 to 5-4-1).

In still another embodiment, the present invention provides an organic electronic element, in which the compound is contained alone, two or more different types of the compounds are contained as a combination, or the compound is contained together with other compounds as a combination of two or more in at least one of the hole injection layer, the hole transport layer, the auxiliary light emitting layer, the light emitting layer, the electron transport layer, and the electron injection layer of the organic material layer. In other words, the compounds corresponding to Formulas 1-1 to 1-9 may be contained alone, a mixture of two or more kinds of compounds of Formulas 1-1 to 1-9 may be contained, or a mixture of the compound of claims and a compound not corresponding to the present invention may be contained in each of the layers. Here, the compounds that do not correspond to the present invention may be a single compound or two or more kinds of compounds. Here, when the compound is contained together with other compounds as a combination of two or more kinds of compounds, another compound may be a compound that is already known for each organic material layer, or a compound to be developed in the future. Here, the compounds contained in the organic material layer may be composed of only the same kind of compounds, or a mixture of two or more kinds of different compounds represented by formula 1-1.

In still another embodiment of the present invention, the present invention provides an organic electronic element further including a light efficiency improvement layer, which is formed on at least one of one side of one surface of the first electrode, which is opposite to the organic material layer and one side of one surface of the second electrode, which is opposite to the organic material layer.

Hereinafter, synthesis examples of the compound represented by Formula 1-1 and manufacturing examples of the organic electronic element according to the present invention will be described in detail by way of example.

However, the following examples are only for illustrative purposes and are not intended to limit the scope of the invention.

### SYNTHESIS EXAMPLES

The product represented by Formula 1-1 according to the present invention is prepared by reaction of Sub 1-1 and Sub 2-1 as in Reaction Scheme 1-1 below, but are not limited thereto.

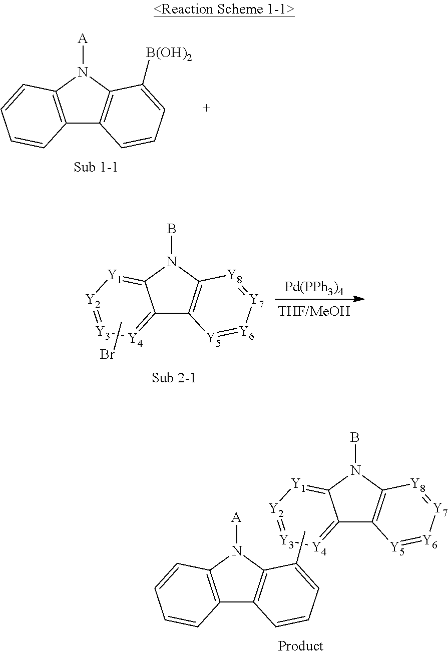

### Synthesis Example of Sub 1-1

Sub 1-1 in Reaction Scheme 1-1 may be synthesized via the reaction pathway of Reaction Scheme 1-2 below, but is not limited thereto.

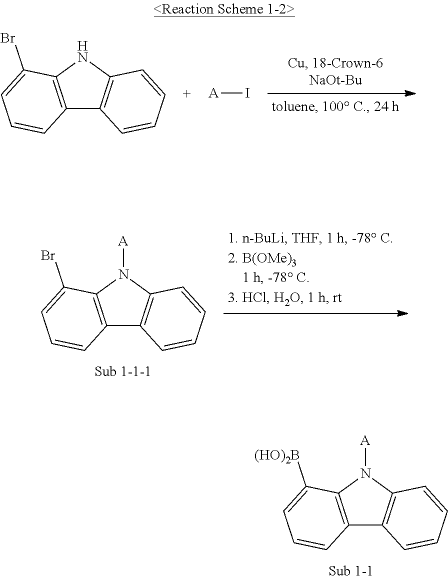

### Synthesis Sub 1-1-1

After bromo-9H-carbazole (203 mmol) and an iodo compound (240 mmol) were mixed with 800 mL of toluene, Cu (764 mg, 12 mmol), 18-Crown-6 (6.3 g, 24 mmol), and NaOt-Bu (57.6 g, 600 mmol) were added thereto, and the mixture was stirred under reflux at 100□ for 24 h. After extraction with ether and water, the organic layer was dried over MgSO₄ and concentrated, and then the generated organic material was subjected to silica gel column chromatography and recrystallization to give an intermediate.

[Synthesis of Sub 1-1(1)-1]

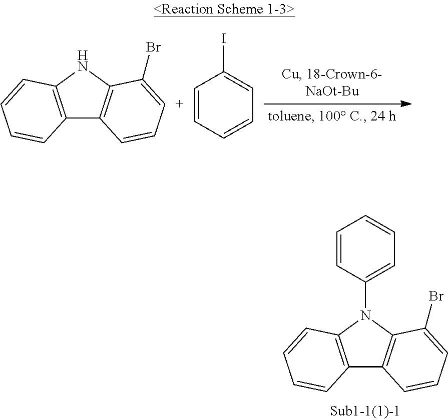 [SMILES: BrC1=[C+]C=Cc2c1[nH]c1ccccc21 ; Ic1ccccc1 ; Brc1cccc2c3ccccc3n(-c3ccccc3)c12]

After bromo-9H-carbazole (50 g, 203 mmol) and iodobenzene (49 g, 240 mmol) were mixed with 800 mL of toluene, Cu (764 mg, 12 mmol), 18-Crown-6 (6.3 g, 24 mmol), and NaOt-Bu (57.6 g, 600 mmol) were added thereto, and the mixture was stirred under reflux at 100□ for 24 h. After extraction with ether and water, the organic layer was dried over MgSO₄ and concentrated, and then the generated organic material was subjected to silica gel column chromatography and recrystallization to give 37.9 g of Sub 1-1(1)-1 (yield: 58%).

Examples of Sub 1-1-1 are as follows, but are limited thereto, and FD-MS values thereof are shown in table 1-1 below.

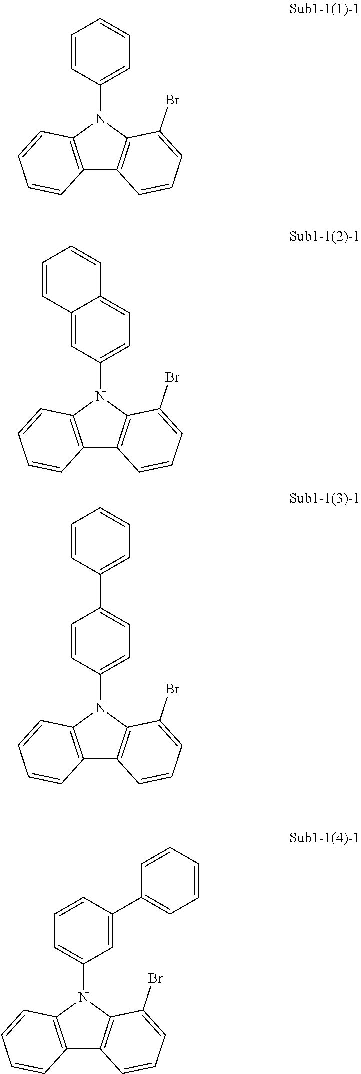 [SMILES: Brc1cccc2c3ccccc3n(-c3ccccc3)c12 ; Brc1cccc2c3ccccc3n(-c3ccc4ccccc4c3)c12 ; Brc1cccc2c3ccccc3n(-c3ccc(-c4ccccc4)cc3)c12 ; Brc1cccc2c3ccccc3n(-c3cccc(-c4ccccc4)c3)c12]

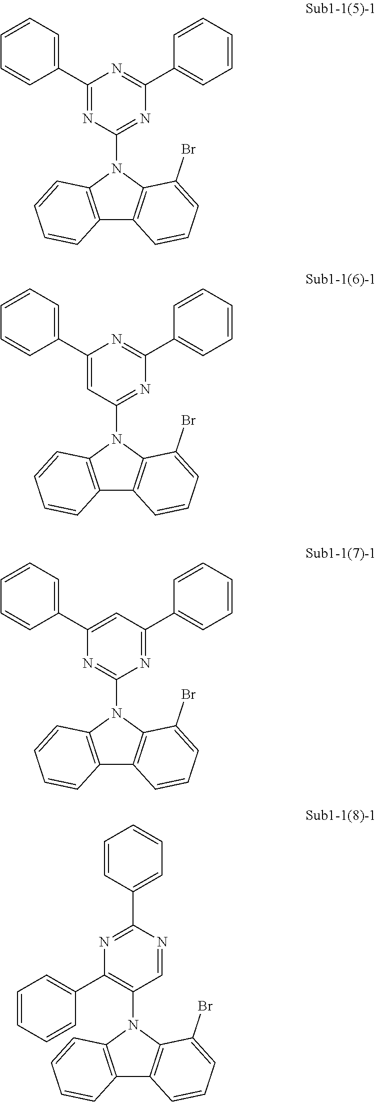

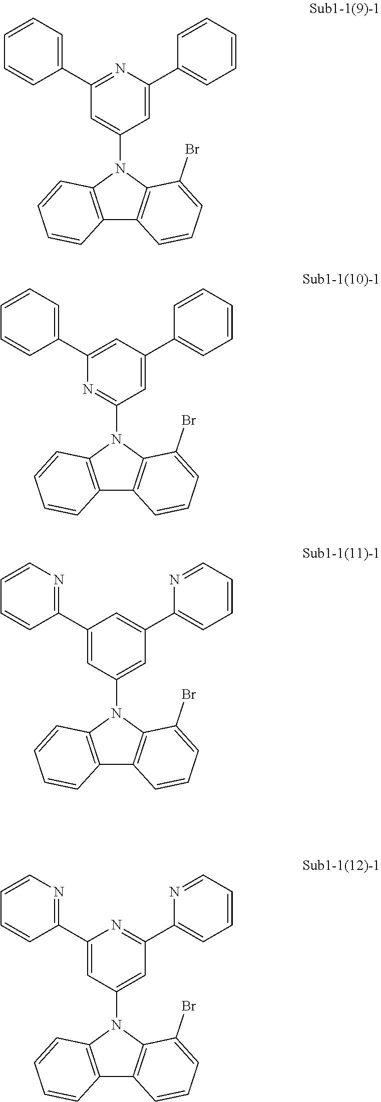 [SMILES: Brc1cccc2c3ccccc3n(-c3cc(-c4ccccc4)nc(-c4ccccc4)c3)c12 ; Brc1cccc2c3ccccc3n(-c3cc(-c4ccccc4)cc(-c4ccccc4)n3)c12 ; Brc1cccc2c3ccccc3n(-c3cc(-c4ccccn4)cc(-c4ccccn4)c3)c12 ; Brc1cccc2c3ccccc3n(-c3cc(-c4ccccn4)nc(-c4ccccn4)c3)c12]

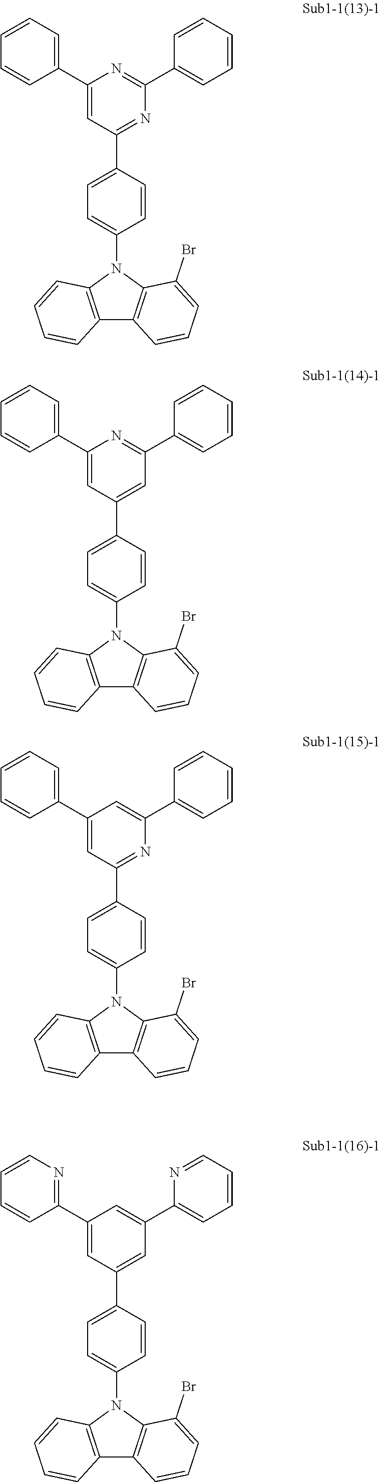 [SMILES: Brc1cccc2c3ccccc3n(-c3ccc(-c4cc(-c5ccccc5)nc(-c5ccccc5)n4)cc3)c12 ; Brc1cccc2c3ccccc3n(-c3ccc(-c4cc(-c5ccccc5)nc(-c5ccccc5)c4)cc3)c12 ; Brc1cccc2c3ccccc3n(-c3ccc(-c4cc(-c5ccccc5)cc(-c5ccccc5)n4)cc3)c12 ; Brc1cccc2c3ccccc3n(-c3ccc(-c4cc(-c5ccccn5)cc(-c5ccccn5)c4)cc3)c12]

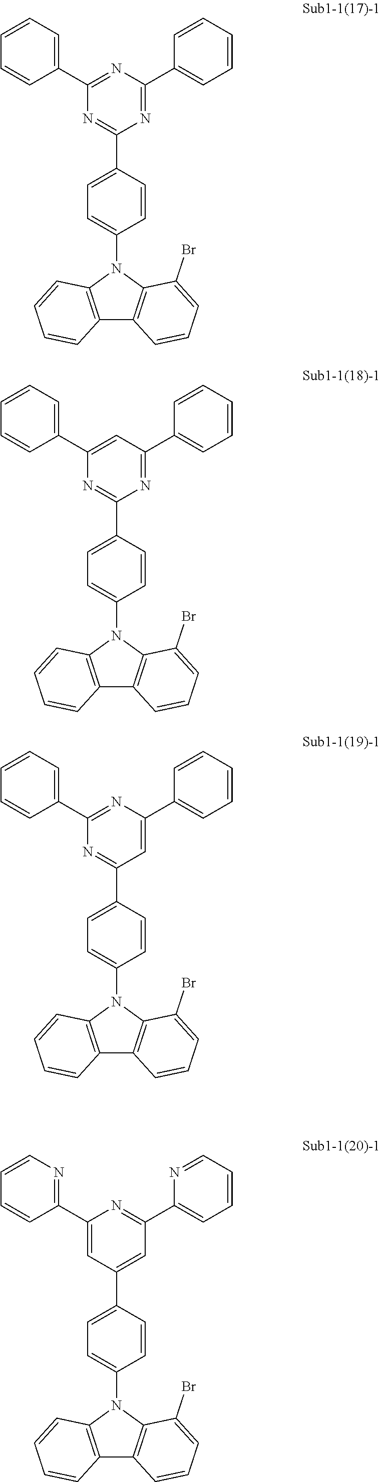 [SMILES: Brc1cccc2c3ccccc3n(-c3ccc(-c4nc(-c5ccccc5)nc(-c5ccccc5)n4)cc3)c12 ; Brc1cccc2c3ccccc3n(-c3ccc(-c4nc(-c5ccccc5)cc(-c5ccccc5)n4)cc3)c12 ; Brc1cccc2c3ccccc3n(-c3ccc(-c4cc(-c5ccccc5)nc(-c5ccccc5)n4)cc3)c12 ; Brc1cccc2c3ccccc3n(-c3ccc(-c4cc(-c5ccccn5)nc(-c5ccccn5)c4)cc3)c12]

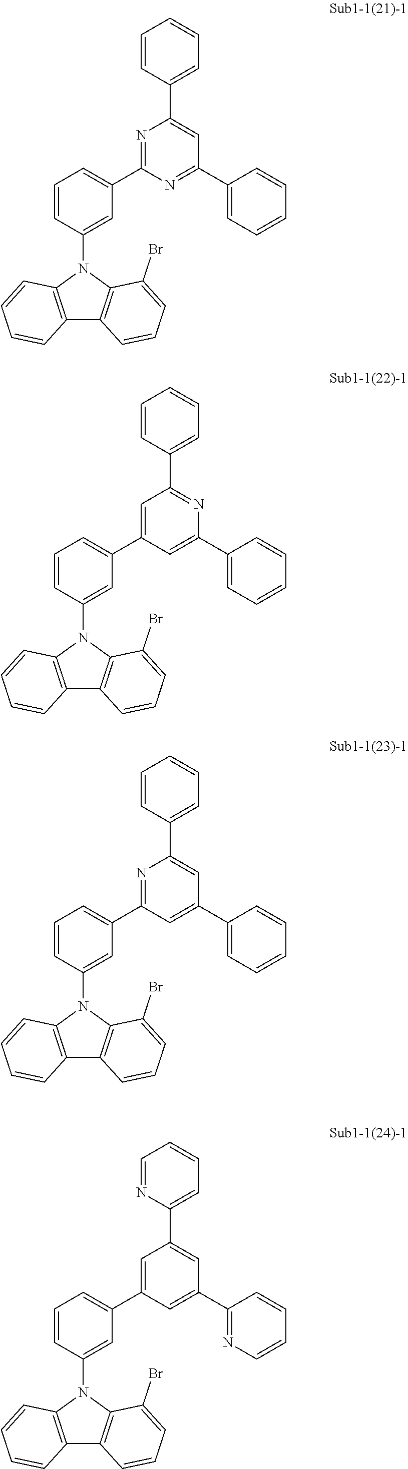 [SMILES: Brc1cccc2c3ccccc3n(-c3cccc(-c4nc(-c5ccccc5)cc(-c5ccccc5)n4)c3)c12 ; Brc1cccc2c3ccccc3n(-c3cccc(-c4cc(-c5ccccc5)nc(-c5ccccc5)c4)c3)c12 ; Brc1cccc2c3ccccc3n(-c3cccc(-c4cc(-c5ccccc5)cc(-c5ccccc5)n4)c3)c12 ; Brc1cccc2c3ccccc3n(-c3cccc(-c4cc(-c5ccccn5)cc(-c5ccccn5)c4)c3)c12]

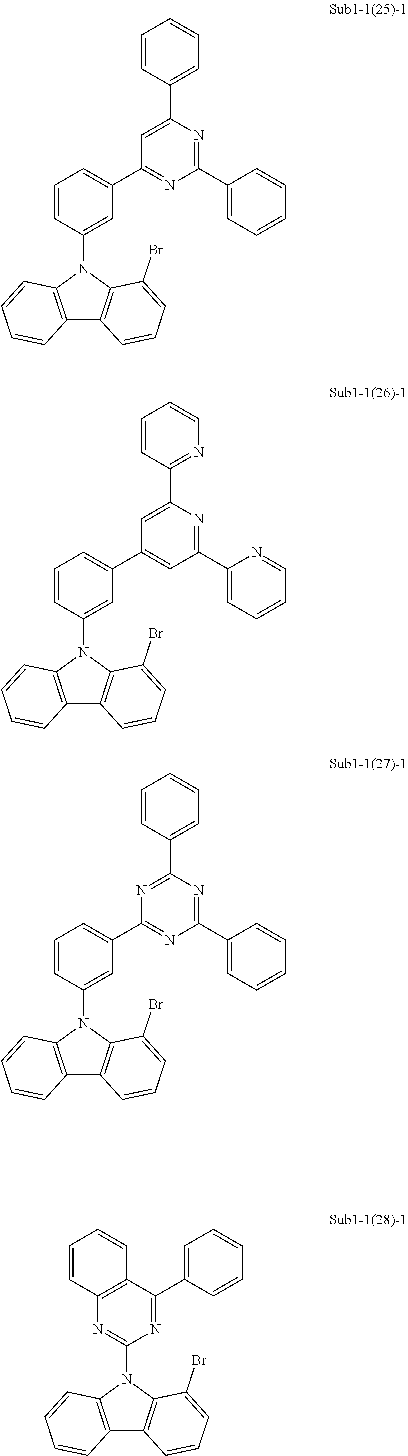 [SMILES: Brc1cccc2c3ccccc3n(-c3cccc(-c4cc(-c5ccccc5)nc(-c5ccccc5)n4)c3)c12 ; Brc1cccc2c3ccccc3n(-c3cccc(-c4cc(-c5ccccn5)nc(-c5ccccn5)c4)c3)c12 ; Brc1cccc2c3ccccc3n(-c3cccc(-c4nc(-c5ccccc5)nc(-c5ccccc5)n4)c3)c12 ; Brc1cccc2c3ccccc3n(-c3nc(-c4ccccc4)c4ccccc4n3)c12]

TABLE 1-1

| Compound | FD-MS | Compound | FD-MS |
|---|---|---|---|
| Sub1-1(1)-1 | m/z = 321.02(C₁₈H₁₂BrN = 322.20) | Sub1-1(2)-1 | m/z = 371.03(C₂₂H₁₄BrN = 372.26) |
| Sub1-1(3)-1 | m/z = 397.05(C₂₄H₁ [symbol] BrN = 398.29) | Sub1-1(4)-1 | m/z = 397.05(C₂₄H₁₆BrN = 398.29) |
| Sub1-1(5)-1 | m/z = 476.06(C₂₇H₁₇BrN₄ = 477.35) | Sub1-1(6)-1 | m/z = 475.07(C₂ [symbol] H₁₈BrN₃ = 476.37) |
| Sub1-1(7)-1 | m/z = 475.07(C₂₈H [symbol] BrN₃ = 476.37) | Sub1-1(8)-1 | m/z = 475.07(C₂ [symbol] H₁ [symbol] BrN₃ = 476.37) |
| Sub1-1(9)-1 | m/z = 474.07(C₂₉H [symbol] BrN₂ = 475.38) | Sub1-1(10)-1 | m/z = 474.07(C₂₉H₁₉BrN₂ = 475.38) |
| Sub1-1(11)-1 | m/z = 475.07(C₂₈H₁₈BrN₃ = 476.37) | Sub1-1(12)-1 | m/z = 476.06(C₂₇H₁₇BrN₄ = 477.35) |
| Sub1-1(13)-1 | m/z = 551.10(C₃₄H₂₂BrN₃ = 552.46) | Sub1-1(14)-1 | m/z = 550.10(C₃₅H₂₃BrN₂ = 551.47) |
| Sub1-1(15)-1 | m/z = 550.10(C₃₅H₂₃BrN₂ = 551.47) | Sub1-1(16)-1 | m/z = 551.10(C₃₄H₂₂BrN₃ = 552.46) |
| Sub1-1(17)-1 | m/z = 552.09(C₃ [symbol] H₂₁BrN₄ = 553.45) | Sub1-1(18)-1 | m/z = 551.10(C₃₄H₂₂BrN₃ = 552.46) |
| Sub1-1(19)-1 | m/z = 551.10(C₃₄H [symbol] BrN₃ = 552.46) | Sub1-1(20)-1 | m/z = 552.09(C [symbol] H₂₁BrN₄ = 553.45) |
| Sub1-1(21)-1 | m/z = 551.10(C₃₄H₂₂BrN₃ = 552.46) | Sub1-1(22)-1 | m/z = 550.10(C₃₅H₂₃BrN₂ = 551.47) |
| Sub1-1(23)-1 | m/z = 550.10(C [symbol] H₂₃BrN₂ = 551.47) | Sub1-1(24)-1 | m/z = 551.10(C [symbol] ₄H₂₂BrN₃ = 552.46) |
| Sub1-1(25)-1 | m/z = 551.10(C₃₄H₂₂BrN [symbol] = 552.46) | Sub1-1(26)-1 | m/z = 552.09(C [symbol] ₃H₂₂BrN₄ = 553.45) |
| Sub1-1(27)-1 | m/z = 552.09(C₃₃H₂₁BrN₄ = 553.45) | Sub1-1(28)-1 | m/z = 449.05(C₂₆H₁₆BrN₃ = 450.33) |

[symbol]

indicates data missing or illegible when filed

### Synthesis of Sub 1-1

A two-necked RBF was equipped with a dropping-funnel, and the product was dissolved in 500 ml of THF and the temperature was maintained at −78□. After stirring for 1 h, trimethoxyborate was slowly added dropwise, followed by again stirring for 1 h. Upon the completion of the reaction, 500 ml of 5% hydrochloric acid was added, followed by stirring at room temperature for 1 h, extraction with water and ethyl acetate, concentration, and recrystallization with MC and Hexane, thereby obtaining compound Sub 1-1.

[Synthesis of Sub 1(1)-1]

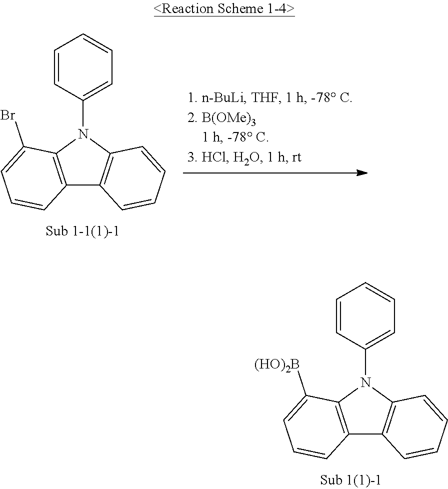 [SMILES: Brc1cccc2c3ccccc3n(-c3ccccc3)c12 ; OB(O)c1cccc2c3ccccc3n(-c3ccccc3)c12]

A two-necked RBF was equipped with a dropping-funnel, and Sub 1(1)-1 (38 g, 118 mmol) was dissolved in 500 ml of THF and the temperature was maintained at −78□. After stirring for 1 h, trimethoxyborate (18.4 g, 177 mmol) was slowly added dropwise, followed by again stirring for 1 h. Upon the completion of the reaction, 500 ml of 5% hydrochloric acid was added, followed by stirring at room temperature for 1 h, extraction with water and ethyl acetate, concentration, and recrystallization with MC and Hexane, thereby obtaining 21 g of compound Sub 1(1)-1 (yield: 62%).

Examples of Sub 1-1 are as follows, but are limited thereto, and FD-MS values thereof are shown in table 1-2 below.

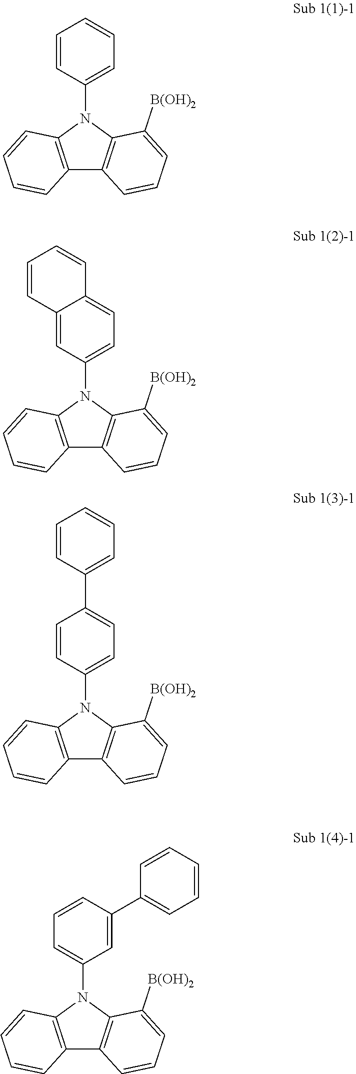 [SMILES: OB(O)c1cccc2c3ccccc3n(-c3ccccc3)c12 ; OB(O)c1cccc2c3ccccc3n(-c3ccc4ccccc4c3)c12 ; OB(O)c1cccc2c3ccccc3n(-c3ccc(-c4ccccc4)cc3)c12 ; OB(O)c1cccc2c3ccccc3n(-c3cccc(-c4ccccc4)c3)c12]

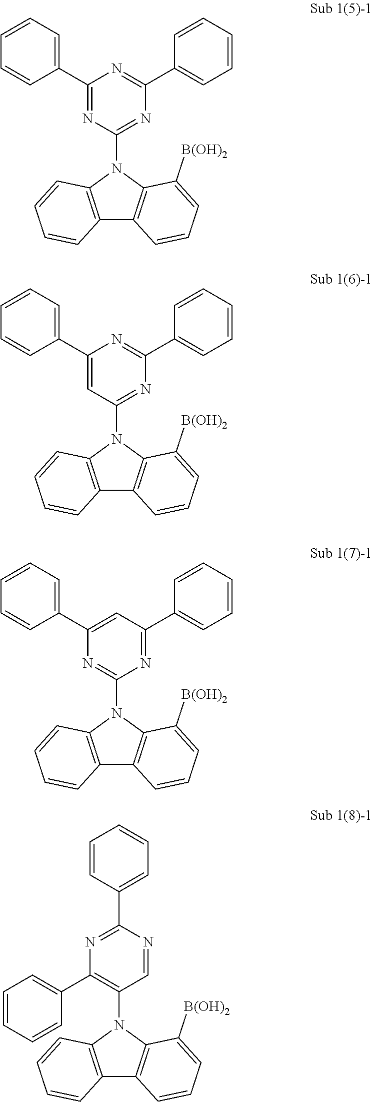 [SMILES: OB(O)c1cccc2c3ccccc3n(-c3nc(-c4ccccc4)nc(-c4ccccc4)n3)c12 ; OB(O)c1cccc2c3ccccc3n(-c3cc(-c4ccccc4)nc(-c4ccccc4)n3)c12 ; OB(O)c1cccc2c3ccccc3n(-c3nc(-c4ccccc4)cc(-c4ccccc4)n3)c12 ; OB(O)c1cccc2c3ccccc3n(-c3cnc(-c4ccccc4)nc3-c3ccccc3)c12]

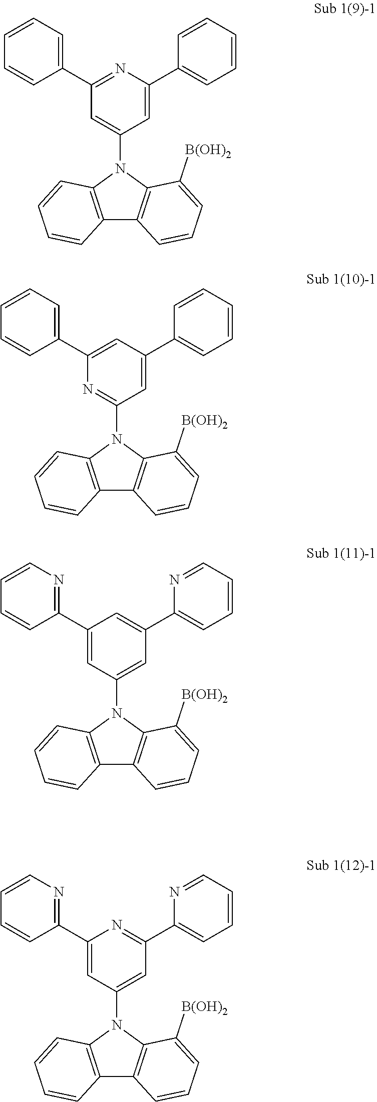 [SMILES: OB(O)c1cccc2c3ccccc3n(-c3cc(-c4ccccc4)nc(-c4ccccc4)c3)c12 ; OB(O)c1cccc2c3ccccc3n(-c3cc(-c4ccccc4)cc(-c4ccccc4)n3)c12 ; OB(O)c1cccc2c3ccccc3n(-c3cc(-c4ccccn4)cc(-c4ccccn4)c3)c12 ; OB(O)c1cccc2c3ccccc3n(-c3cc(-c4ccccn4)nc(-c4ccccn4)c3)c12]

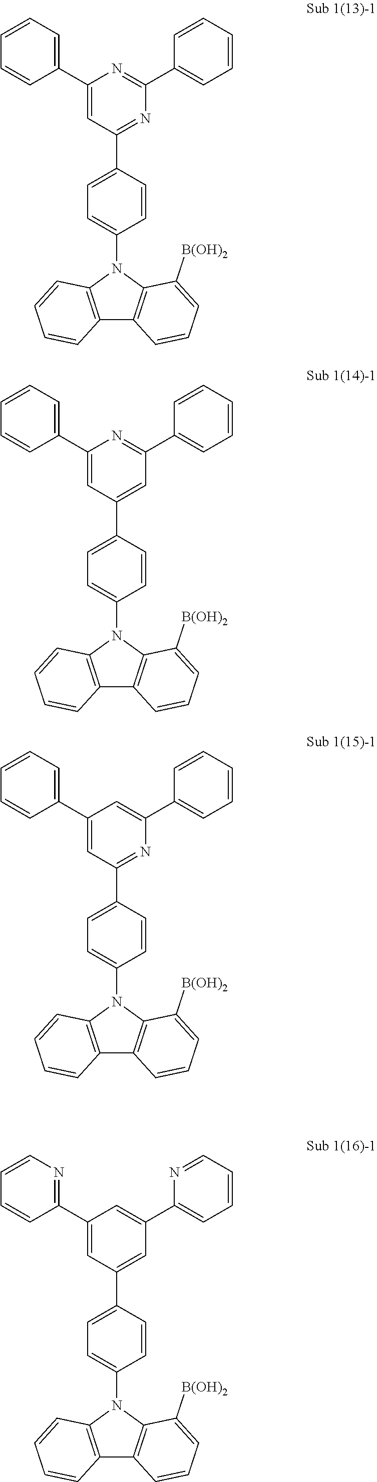 [SMILES: OB(O)c1cccc2c3ccccc3n(-c3ccc(-c4cc(-c5ccccc5)nc(-c5ccccc5)n4)cc3)c12 ; OB(O)c1cccc2c3ccccc3n(-c3ccc(-c4cc(-c5ccccc5)nc(-c5ccccc5)c4)cc3)c12 ; OB(O)c1cccc2c3ccccc3n(-c3ccc(-c4cc(-c5ccccc5)cc(-c5ccccc5)n4)cc3)c12 ; OB(O)c1cccc2c3ccccc3n(-c3ccc(-c4cc(-c5ccccn5)cc(-c5ccccn5)c4)cc3)c12]

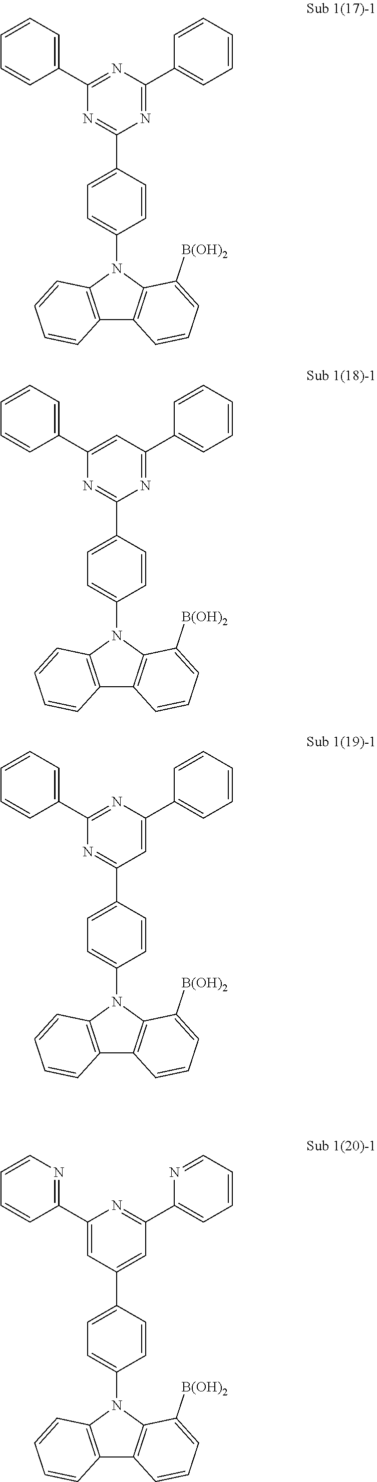 [SMILES: OB(O)c1cccc2c3ccccc3n(-c3ccc(-c4nc(-c5ccccc5)nc(-c5ccccc5)n4)cc3)c12 ; OB(O)c1cccc2c3ccccc3n(-c3ccc(-c4nc(-c5ccccc5)cc(-c5ccccc5)n4)cc3)c12 ; OB(O)c1cccc2c3ccccc3n(-c3ccc(-c4cc(-c5ccccc5)nc(-c5ccccc5)n4)cc3)c12 ; OB(O)c1cccc2c3ccccc3n(-c3ccc(-c4cc(-c5ccccn5)nc(-c5ccccn5)c4)cc3)c12]

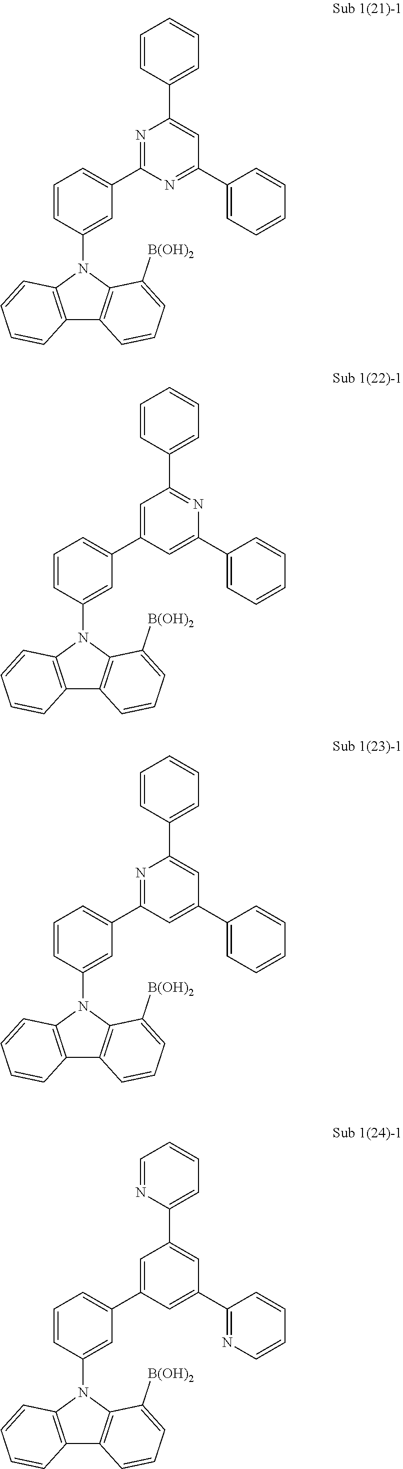 [SMILES: OB(O)c1cccc2c3ccccc3n(-c3cccc(-c4nc(-c5ccccc5)cc(-c5ccccc5)n4)c3)c12 ; OB(O)c1cccc2c3ccccc3n(-c3cccc(-c4cc(-c5ccccc5)nc(-c5ccccc5)c4)c3)c12 ; OB(O)c1cccc2c3ccccc3n(-c3cccc(-c4cc(-c5ccccc5)cc(-c5ccccc5)n4)c3)c12 ; OB(O)c1cccc2c3ccccc3n(-c3cccc(-c4cc(-c5ccccn5)cc(-c5ccccn5)c4)c3)c12]

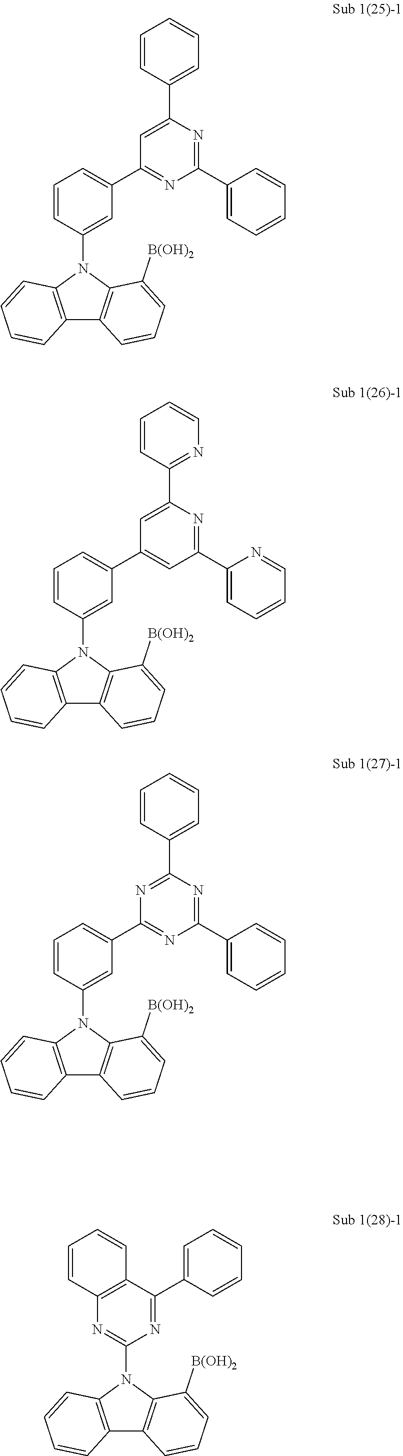 [SMILES: OB(O)c1cccc2c3ccccc3n(-c3cccc(-c4cc(-c5ccccc5)nc(-c5ccccc5)n4)c3)c12 ; OB(O)c1cccc2c3ccccc3n(-c3cccc(-c4cc(-c5ccccn5)nc(-c5ccccn5)c4)c3)c12 ; OB(O)c1cccc2c3ccccc3n(-c3cccc(-c4nc(-c5ccccc5)nc(-c5ccccc5)n4)c3)c12 ; OB(O)c1cccc2c3ccccc3n(-c3nc(-c4ccccc4)c4ccccc4n3)c12]

TABLE 1-2

| Compound | FD-MS | Compound | FD-MS |
|---|---|---|---|
| Sub 1(1)-1 | m/z = 287.11(C₁₈H₁₄BNO₂ = 287.12) | Sub 1(2)-1 | m/z = 337.13(C₂₂H₁₆BNO₂ = 337.18) |
| Sub 1(3)-1 | m/z = 363.14(C₂₄H₁₈BNO₂ = 363.22) | Sub 1(4)-1 | m/z = 363.14(C₂₄H₁₈BNO₂ = 363.22) |
| Sub 1(5)-1 | m/z = 442.16(C₂₇H₁₉BN₄O₂ = 442.28) | Sub 1(6)-1 | m/z = 441.16(C₂ [symbol] H₂₀BN₃O₂ = 441.29) |
| Sub 1(7)-1 | m/z = 441.16(C₂₈H₂₀BN₃O₂ = 441.29) | Sub 1(8)-1 | m/z = 441.16(C₂₈H₂₀BN₃O₂ = 441.29) |
| Sub 1(9)-1 | m/z = 440.17(C₂₉H₂₁BN₂O₂ = 440.30) | Sub 1(10)-1 | m/z = 440.17(C₂₉H₂₁BN₂O₂ = 440.30) |
| Sub 1(11)-1 | m/z = 441.16(C₂₈H₂₀BN₃O₂ = 441.29) | Sub 1(12)-1 | m/z = 442.16(C₂₇H₁₉BN₄O₂ = 442.28) |
| Sub 1(13)-1 | m/z = 517.20(C₃₄H₂₄BN₃O₂ = 517.38) | Sub 1(14)-1 | m/z = 516.20(C₃ [symbol] H₂₅BN₂O₂ = 516.40) |
| Sub 1(15)-1 | m/z = 516.20(C₃₅H₂₅BN₂O₂ = 516.40) | Sub 1(16)-1 | m/z = 517.20(C₃₄H₂₄BN₃O₂ = 517.38) |
| Sub 1(17)-1 | m/z = 518.19(C₃₃H₂₃BN₄O₂ = 518.37) | Sub 1(18)-1 | m/z = 517.20(C [symbol] H₂₄BN₃O₂ = 517.38) |
| Sub 1(19)-1 | m/z = 517.20(C₃₄H₂₄BN₃O₂ = 517.38) | Sub 1(20)-1 | m/z = 518.19(C₃₃H₂₃BN₄O₂ = 518.37) |
| Sub 1(21)-1 | m/z = 517.20(C₃₄H₂₄BN₃O₂ = 517.38) | Sub 1(22)-1 | m/z = 516.20(C₃₅H₂₅BN₂O₂ = 516.40) |
| Sub 1(23)-1 | m/z = 516.20(C₃₅H₂₅BN₂O₂ = 516.40) | Sub 1(24)-1 | m/z = 517.20(C₃₄H₂₄BN₃O₂ = 517.38) |
| Sub 1(25)-1 | m/z = 517.20(C₃₄H₂ [symbol] BN₃O₂ = 517.38) | Sub 1(26)-1 | m/z = 518.19(C₃₃H₂₃BN₄O₂ = 518.37) |
| Sub 1(27)-1 | m/z = 518.19(C₃₃H₂₃BN₄O₂ = 518.37) | Sub 1(28)-1 | m/z = 415.15(C [symbol] H₁₈BN₃O₂ = 415.25) |

[symbol]

indicates data missing or illegible when filed

### Synthesis Example of Sub 1-2

Sub 2-1 in Reaction Scheme 1 may be synthesized via the reaction pathway of Reaction Scheme 1-5 below, but is not limited thereto.

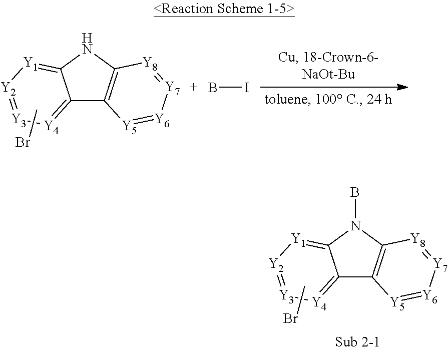

[Synthesis of Sub 1-2-(1)]

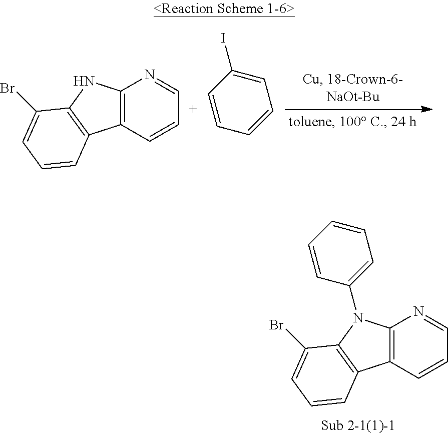

After 8-bromo-9H-pyrido[2,3-b]indole (50.2 g, 203 mmol) and iodobenzene (49.0 g, 240 mmol) were mixed with 800 mL of toluene, Cu (764 mg, 12 mmol), 18-Crown-6 (6.3 g, 24 mmol), and NaOt-Bu (57.6 g, 600 mmol) were added thereto, and the mixture was stirred under reflux at 100□ for 24 h. After extraction with ether and water, the organic layer was dried over MgSO₄ and concentrated, and then the generated organic material was subjected to silica gel column chromatography and recrystallization to give 28.2 g of 8-bromo-9-phenyl-9H-pyrido[2,3-b]indole (yield: 43%).

Examples of Sub 2-1 are as follows, but are limited thereto, and FD-MS values thereof are shown in table 1-3 below.

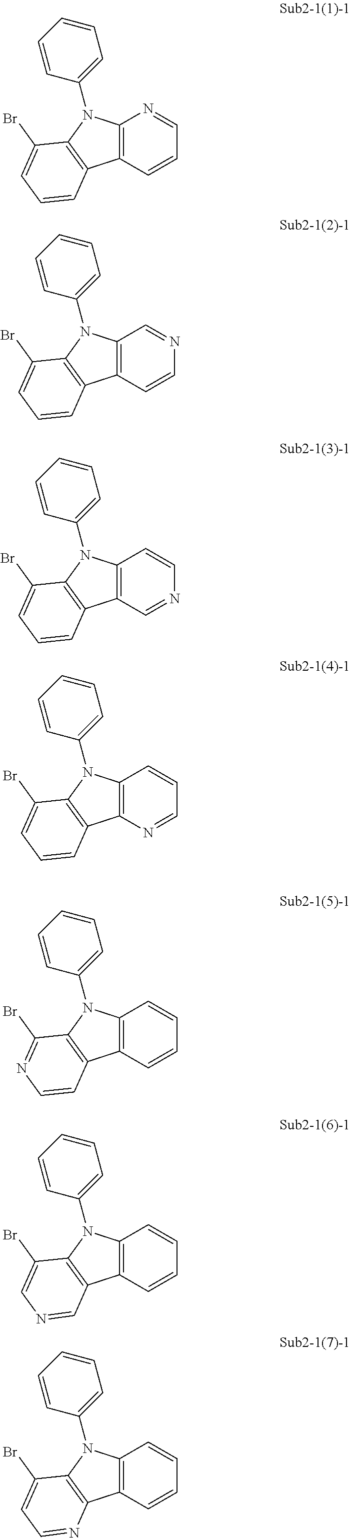

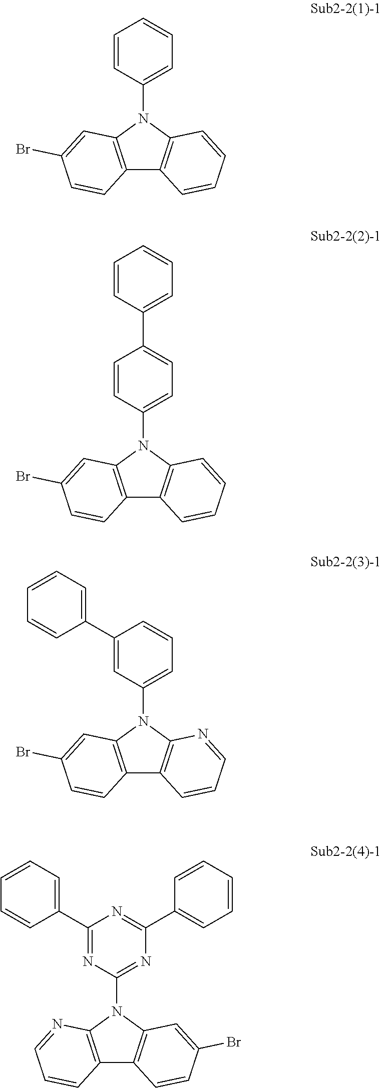 [SMILES: Brc1ccc2c3ccccc3n(-c3ccccc3)c2c1 ; Brc1ccc2c3ccccc3n(-c3ccc(-c4ccccc4)cc3)c2c1 ; Brc1ccc2c3cccnc3n(-c3cccc(-c4ccccc4)c3)c2c1 ; Brc1ccc2c3cccnc3n(-c3nc(-c4ccccc4)nc(-c4ccccc4)n3)c2c1]

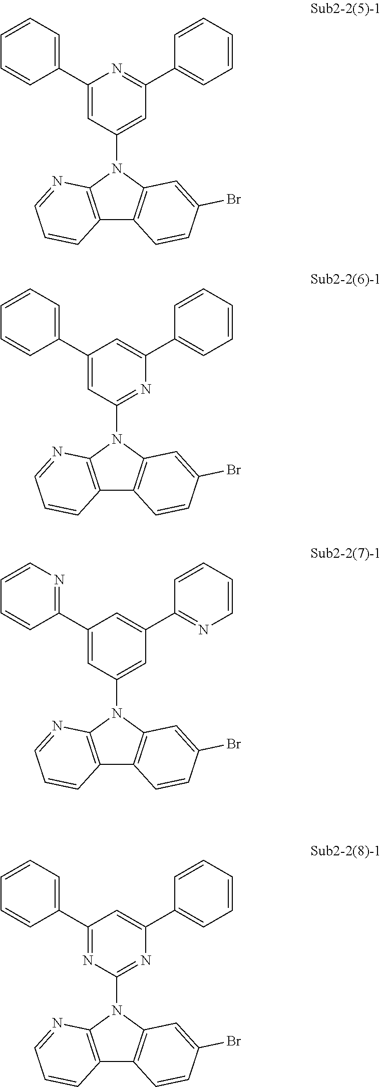 [SMILES: Brc1ccc2c3cccnc3n(-c3cc(-c4ccccc4)nc(-c4ccccc4)c3)c2c1 ; Brc1ccc2c3cccnc3n(-c3cc(-c4ccccc4)cc(-c4ccccc4)n3)c2c1 ; Brc1ccc2c3cccnc3n(-c3cc(-c4ccccn4)cc(-c4ccccn4)c3)c2c1 ; Brc1ccc2c3cccnc3n(-c3nc(-c4ccccc4)cc(-c4ccccc4)n3)c2c1]

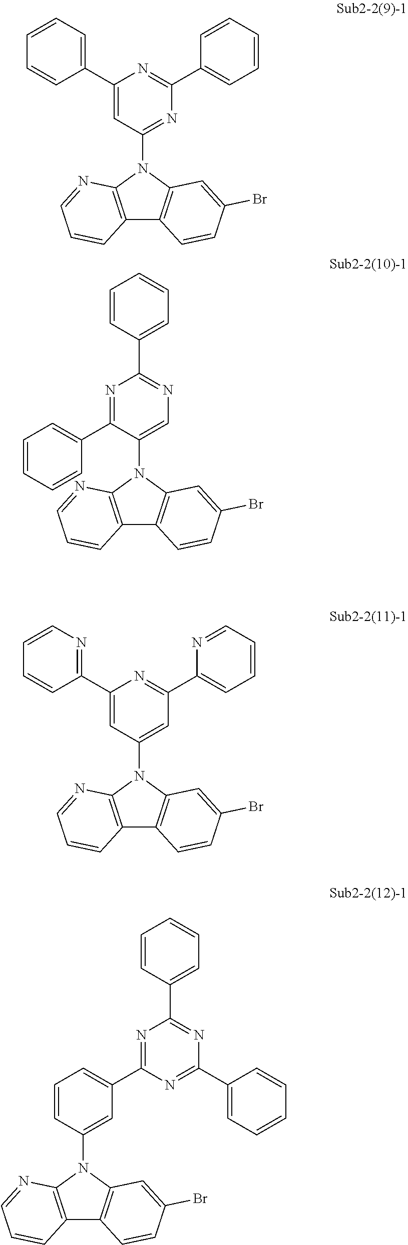

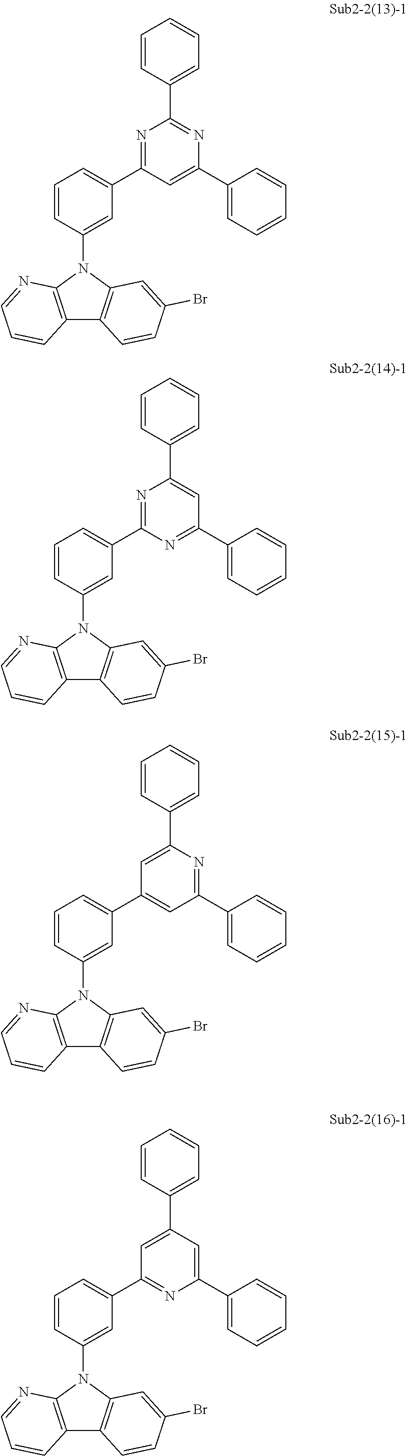

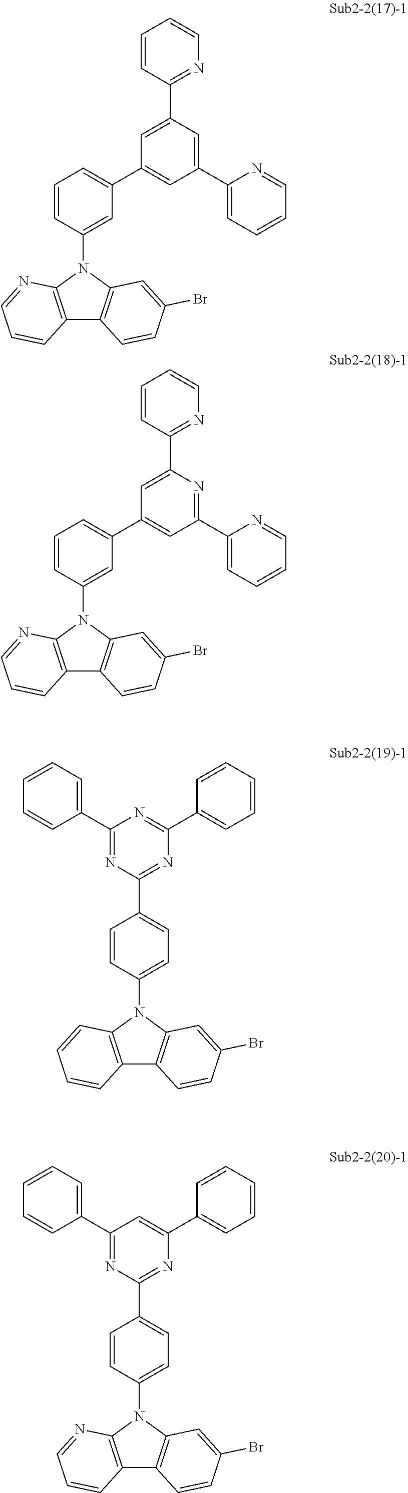

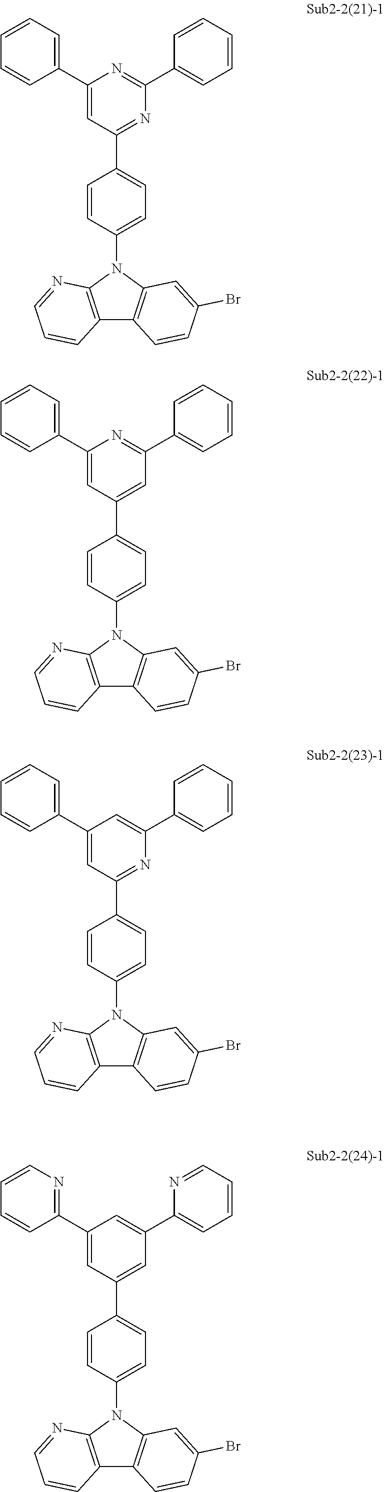 [SMILES: Brc1ccc2c3cccnc3n(-c3ccc(-c4cc(-c5ccccc5)nc(-c5ccccc5)n4)cc3)c2c1 ; Brc1ccc2c3cccnc3n(-c3ccc(-c4cc(-c5ccccc5)nc(-c5ccccc5)c4)cc3)c2c1 ; Brc1ccc2c3cccnc3n(-c3ccc(-c4cc(-c5ccccc5)cc(-c5ccccc5)n4)cc3)c2c1 ; Brc1ccc2c3cccnc3n(-c3ccc(-c4cc(-c5ccccn5)cc(-c5ccccn5)c4)cc3)c2c1]

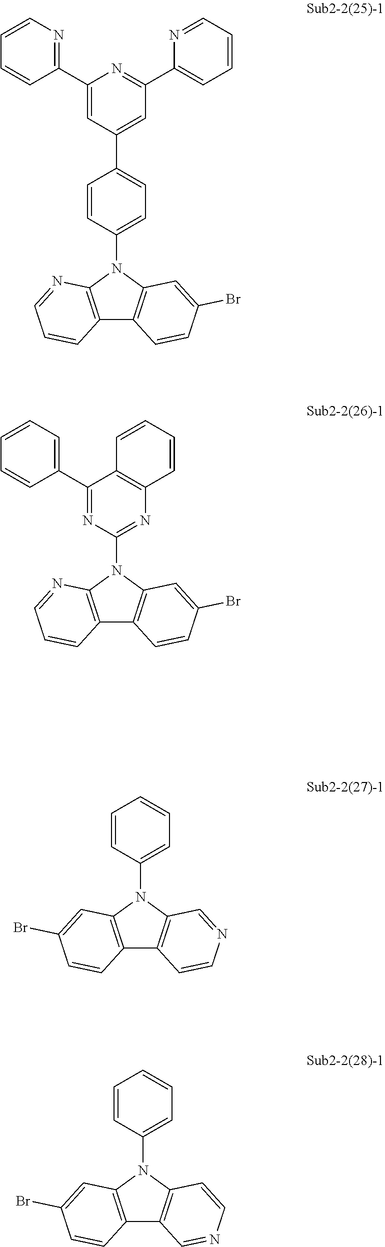 [SMILES: Brc1ccc2c3cccnc3n(-c3ccc(-c4cc(-c5ccccn5)nc(-c5ccccn5)c4)cc3)c2c1 ; Brc1ccc2c3cccnc3n(-c3nc(-c4ccccc4)c4ccccc4n3)c2c1 ; Brc1ccc2c3ccncc3n(-c3ccccc3)c2c1 ; Brc1ccc2c3cnccc3n(-c3ccccc3)c2c1]

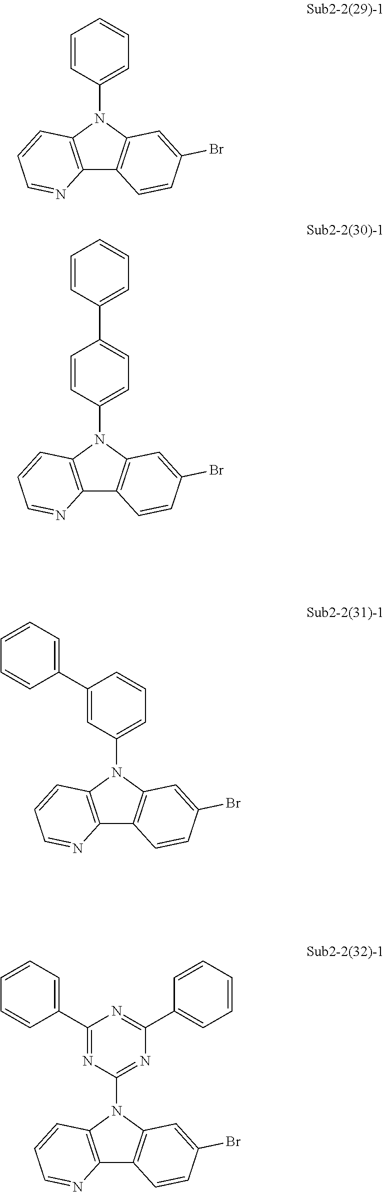 [SMILES: Brc1ccc2c3ncccc3n(-c3ccccc3)c2c1 ; Brc1ccc2c3ncccc3n(-c3ccc(-c4ccccc4)cc3)c2c1 ; Brc1ccc2c3ncccc3n(-c3cccc(-c4ccccc4)c3)c2c1 ; Brc1ccc2c3ncccc3n(-c3nc(-c4ccccc4)nc(-c4ccccc4)n3)c2c1]

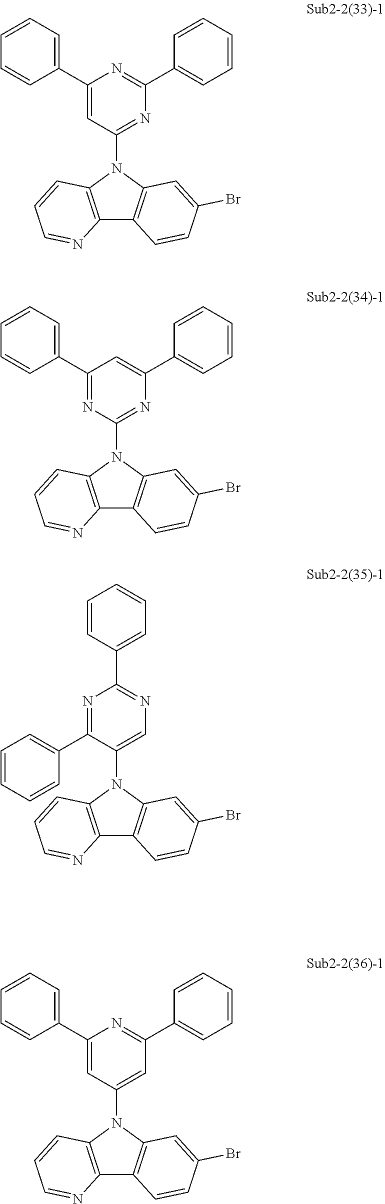 [SMILES: Brc1ccc2c3ncccc3n(-c3cc(-c4ccccc4)nc(-c4ccccc4)n3)c2c1 ; Brc1ccc2c3ncccc3n(-c3nc(-c4ccccc4)cc(-c4ccccc4)n3)c2c1 ; Brc1ccc2c3ncccc3n(-c3cnc(-c4ccccc4)nc3-c3ccccc3)c2c1 ; Brc1ccc2c3ncccc3n(-c3cc(-c4ccccc4)nc(-c4ccccc4)c3)c2c1]

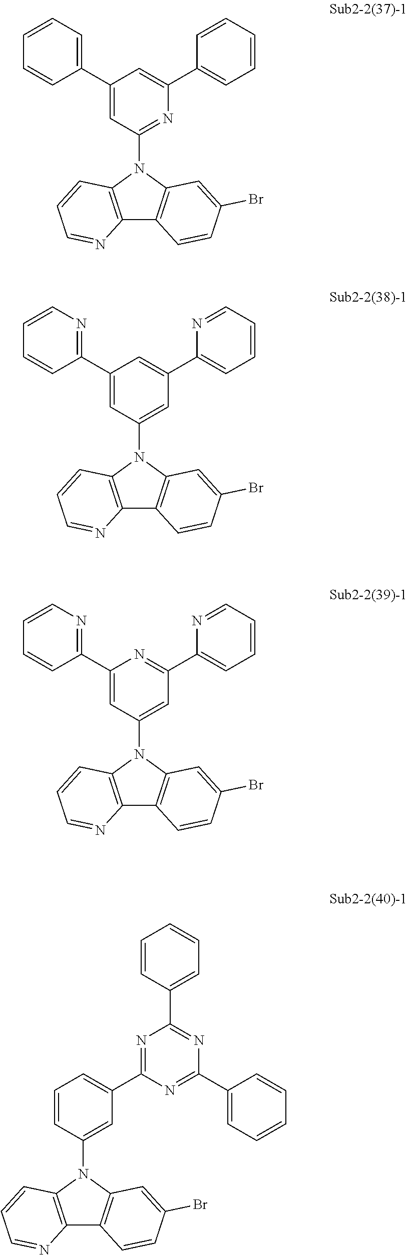 [SMILES: Brc1ccc2c3ncccc3n(-c3cc(-c4ccccc4)cc(-c4ccccc4)n3)c2c1 ; Brc1ccc2c3ncccc3n(-c3cc(-c4ccccn4)cc(-c4ccccn4)c3)c2c1 ; Brc1ccc2c3ncccc3n(-c3cc(-c4ccccn4)nc(-c4ccccn4)c3)c2c1 ; Brc1ccc2c3ncccc3n(-c3cccc(-c4nc(-c5ccccc5)nc(-c5ccccc5)n4)c3)c2c1]

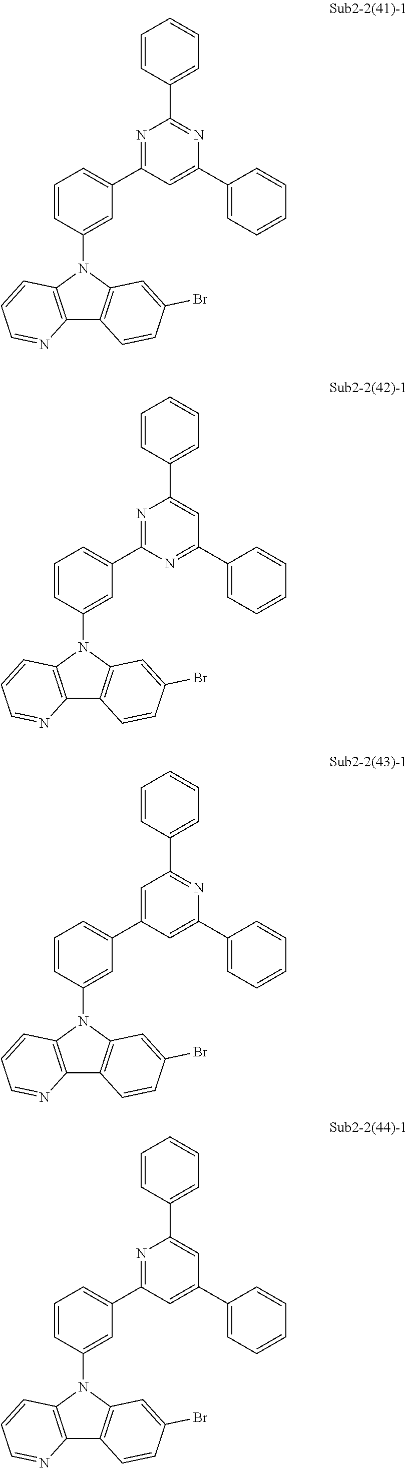 [SMILES: Brc1ccc2c3ncccc3n(-c3cccc(-c4cc(-c5ccccc5)nc(-c5ccccc5)n4)c3)c2c1 ; Brc1ccc2c3ncccc3n(-c3cccc(-c4nc(-c5ccccc5)cc(-c5ccccc5)n4)c3)c2c1 ; Brc1ccc2c3ncccc3n(-c3cccc(-c4cc(-c5ccccc5)nc(-c5ccccc5)c4)c3)c2c1 ; Brc1ccc2c3ncccc3n(-c3cccc(-c4cc(-c5ccccc5)cc(-c5ccccc5)n4)c3)c2c1]

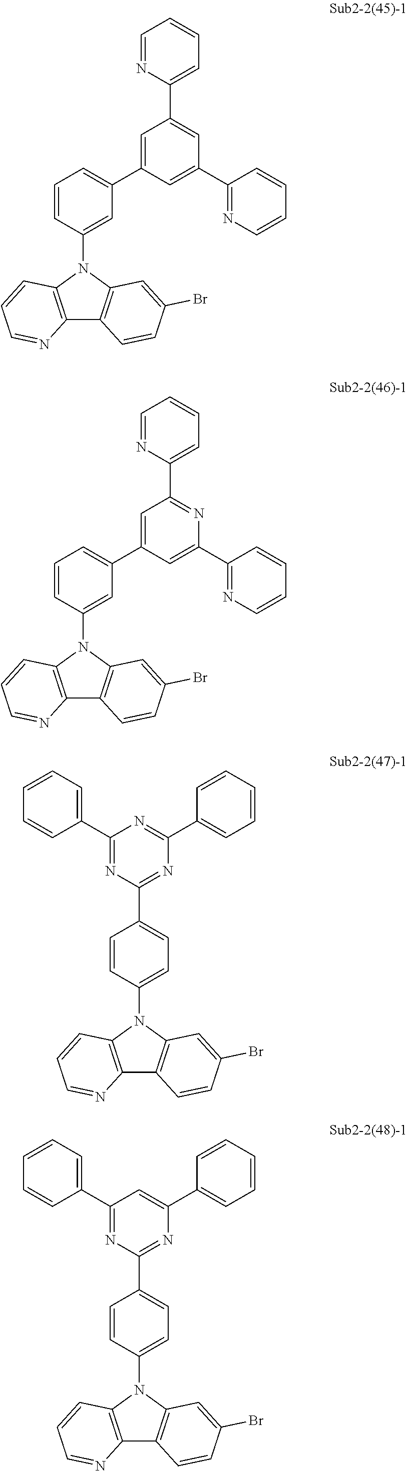 [SMILES: Brc1ccc2c3ncccc3n(-c3cccc(-c4cc(-c5ccccn5)cc(-c5ccccn5)c4)c3)c2c1 ; Brc1ccc2c3ncccc3n(-c3cccc(-c4cc(-c5ccccn5)nc(-c5ccccn5)c4)c3)c2c1 ; Brc1ccc2c3ncccc3n(-c3ccc(-c4nc(-c5ccccc5)nc(-c5ccccc5)n4)cc3)c2c1 ; Brc1ccc2c3ncccc3n(-c3ccc(-c4nc(-c5ccccc5)cc(-c5ccccc5)n4)cc3)c2c1]

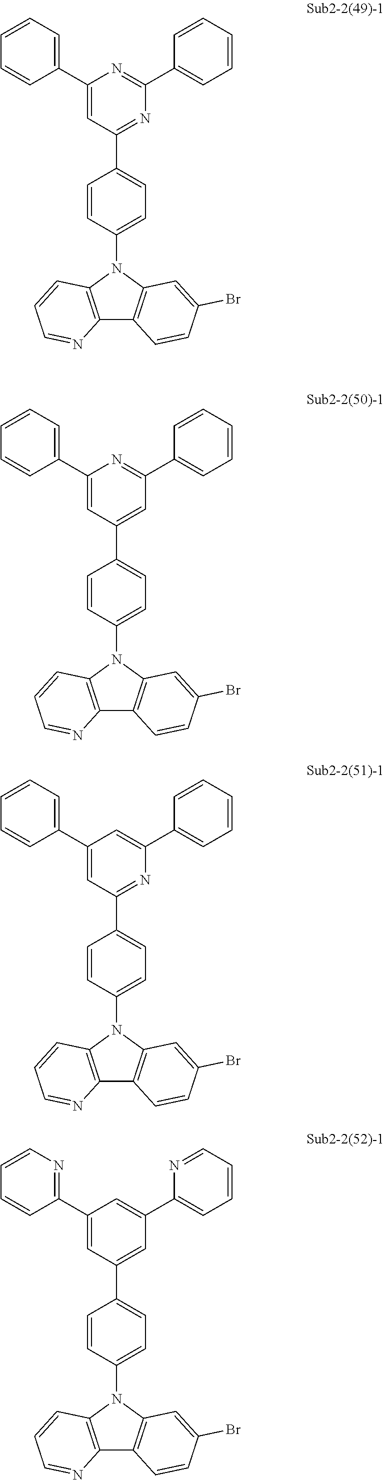 [SMILES: Brc1ccc2c3ncccc3n(-c3ccc(-c4cc(-c5ccccc5)nc(-c5ccccc5)n4)cc3)c2c1 ; Brc1ccc2c3ncccc3n(-c3ccc(-c4cc(-c5ccccc5)nc(-c5ccccc5)c4)cc3)c2c1 ; Brc1ccc2c3ncccc3n(-c3ccc(-c4cc(-c5ccccc5)cc(-c5ccccc5)n4)cc3)c2c1 ; Brc1ccc2c3ncccc3n(-c3ccc(-c4cc(-c5ccccn5)cc(-c5ccccn5)c4)cc3)c2c1]

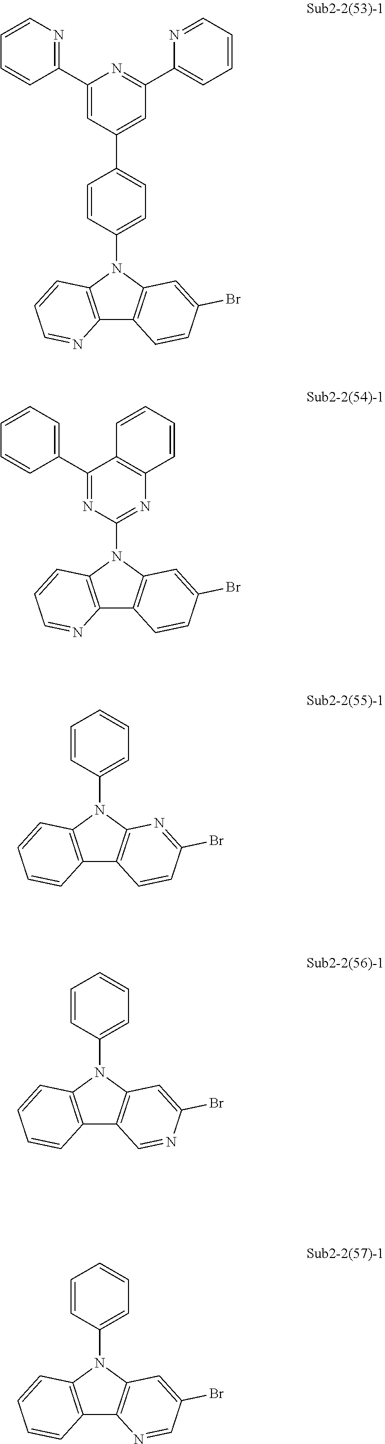 [SMILES: Brc1ccc2c3ncccc3n(-c3ccc(-c4cc(-c5ccccn5)nc(-c5ccccn5)c4)cc3)c2c1 ; Brc1ccc2c3ncccc3n(-c3nc(-c4ccccc4)c4ccccc4n3)c2c1 ; Brc1ccc2c3ccccc3n(-c3ccccc3)c2n1 ; Brc1cc2c(cn1)c1ccccc1n2-c1ccccc1 ; Brc1cnc2c3ccccc3n(-c3ccccc3)c2c1]

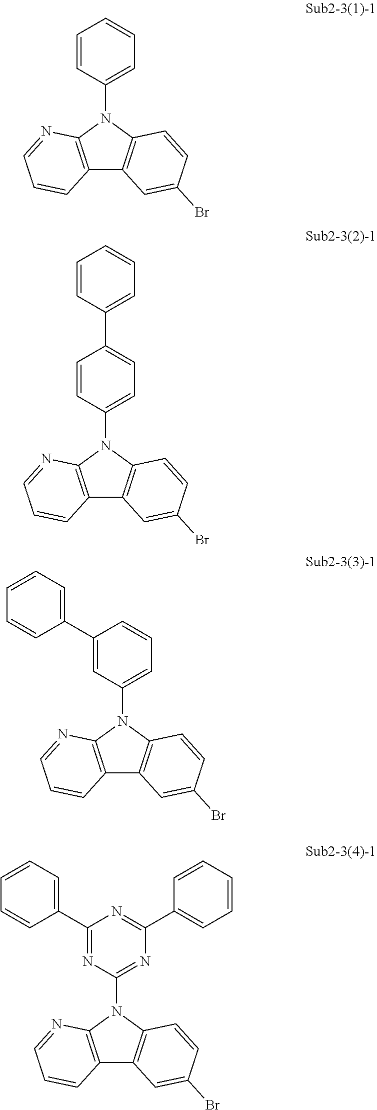 [SMILES: Brc1ccc2c(c1)c1cccnc1n2-c1ccccc1 ; Brc1ccc2c(c1)c1cccnc1n2-c1ccc(-c2ccccc2)cc1 ; Brc1ccc2c(c1)c1cccnc1n2-c1cccc(-c2ccccc2)c1 ; Brc1ccc2c(c1)c1cccnc1n2-c1nc(-c2ccccc2)nc(-c2ccccc2)n1]

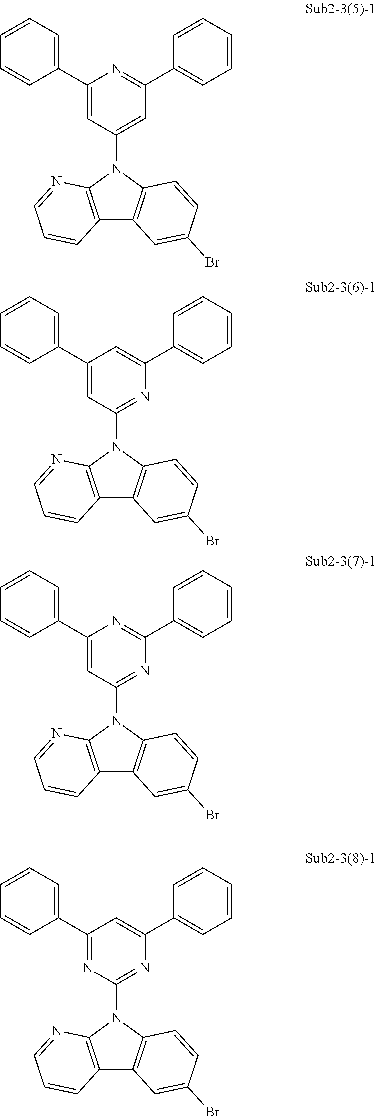

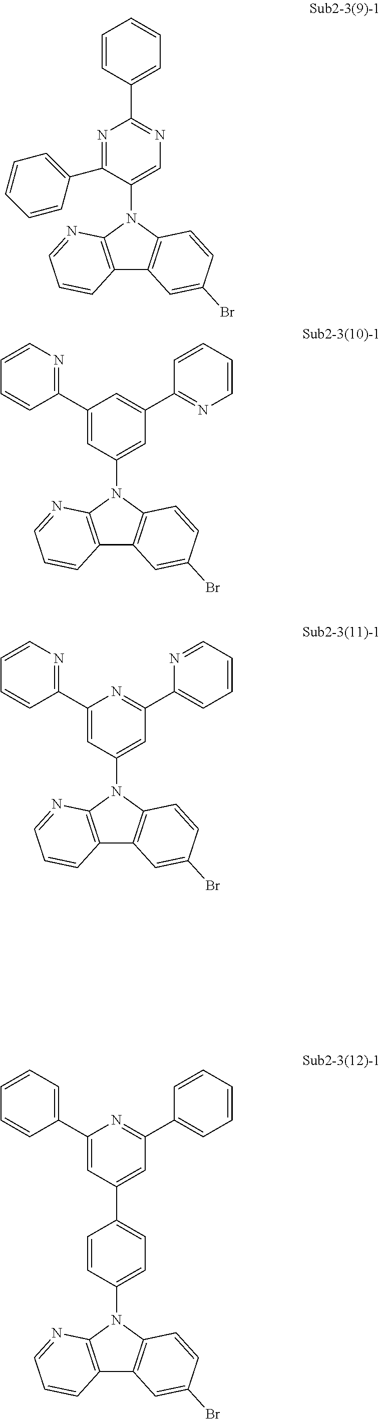

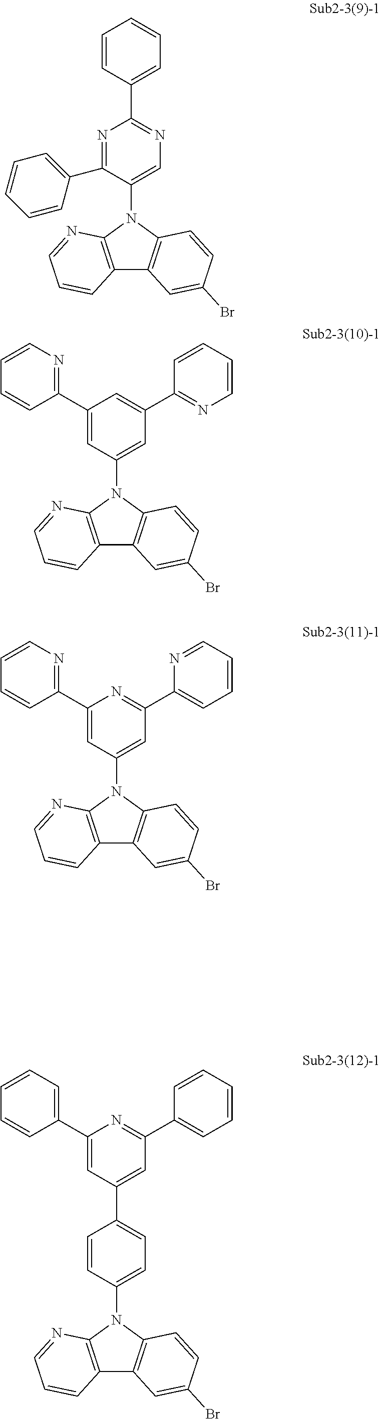

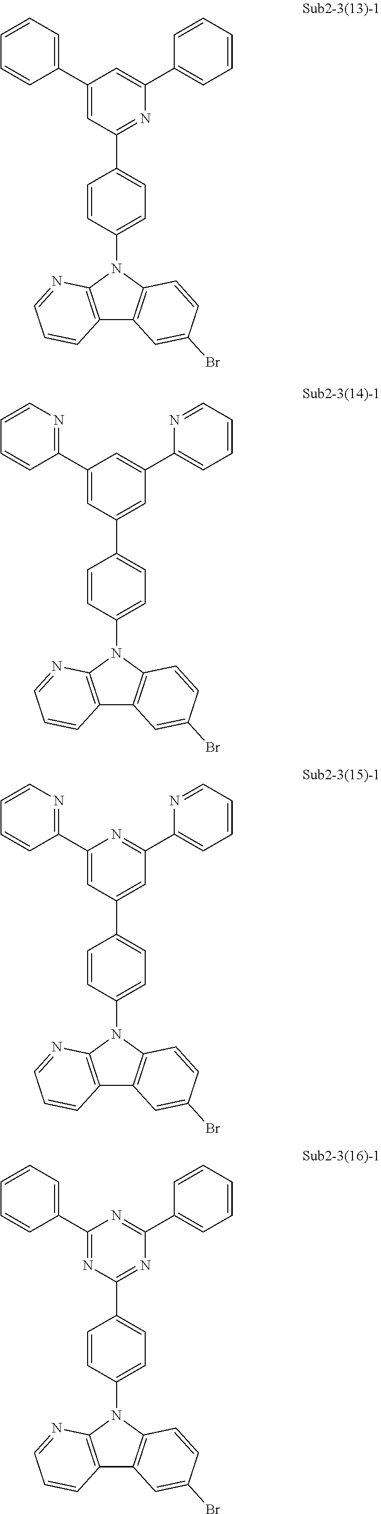

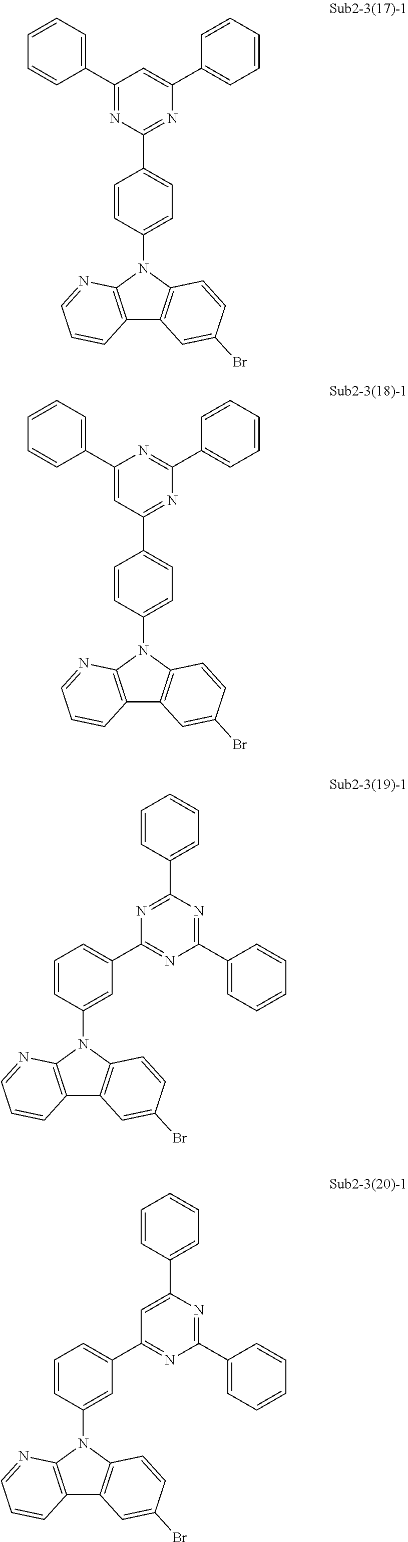

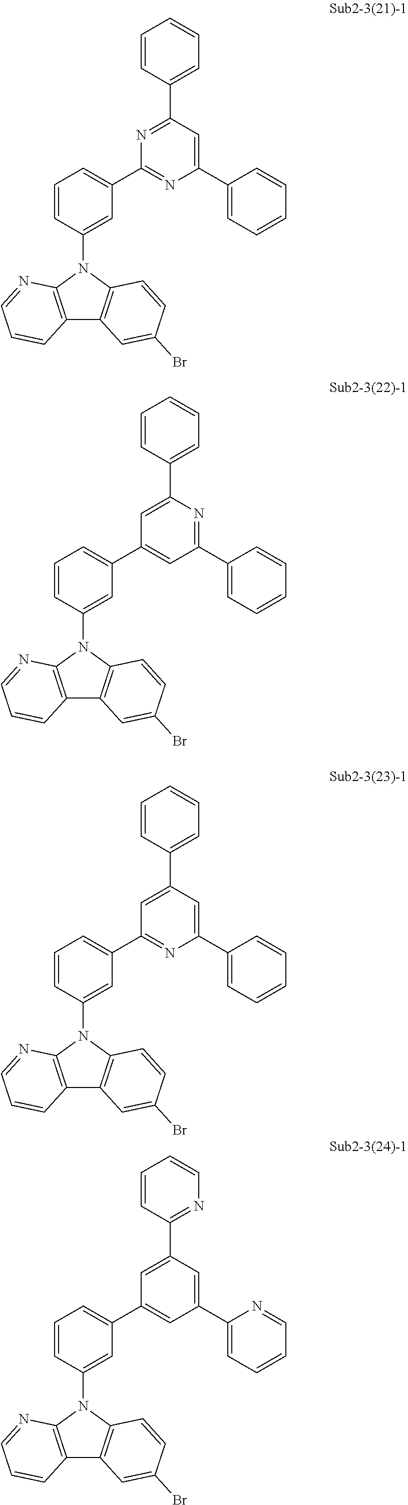

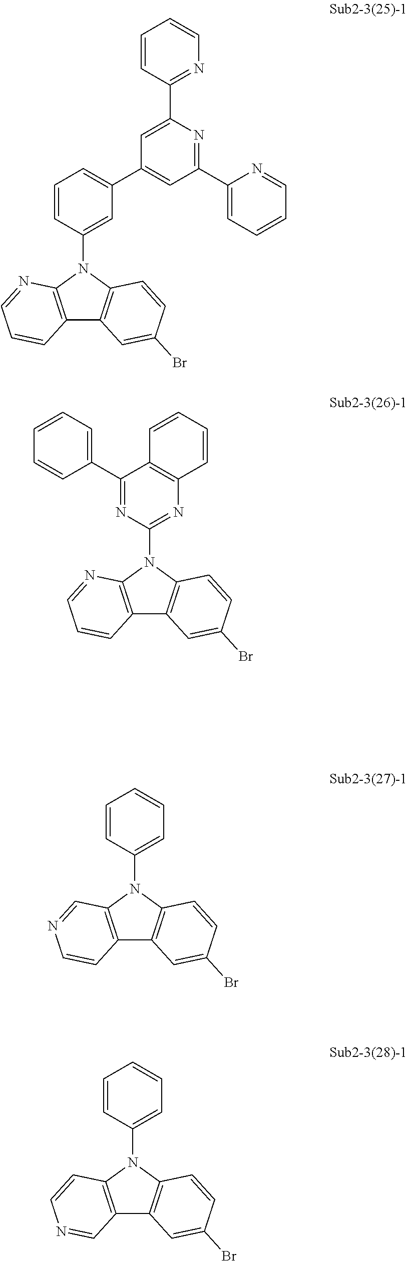 [SMILES: Brc1ccc2c(c1)c1cccnc1n2-c1cccc(-c2cc(-c3ccccn3)nc(-c3ccccn3)c2)c1 ; Brc1ccc2c(c1)c1cccnc1n2-c1nc(-c2ccccc2)c2ccccc2n1 ; Brc1ccc2c(c1)c1ccncc1n2-c1ccccc1 ; Brc1ccc2c(c1)c1cnccc1n2-c1ccccc1]

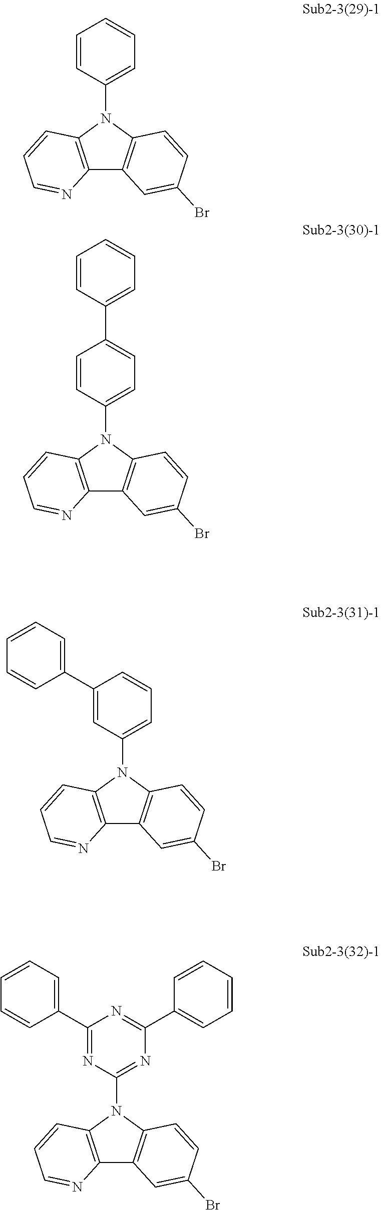 [SMILES: Brc1ccc2c(c1)c1ncccc1n2-c1ccccc1 ; Brc1ccc2c(c1)c1ncccc1n2-c1ccc(-c2ccccc2)cc1 ; Brc1ccc2c(c1)c1ncccc1n2-c1cccc(-c2ccccc2)c1 ; Brc1ccc2c(c1)c1ncccc1n2-c1nc(-c2ccccc2)nc(-c2ccccc2)n1]

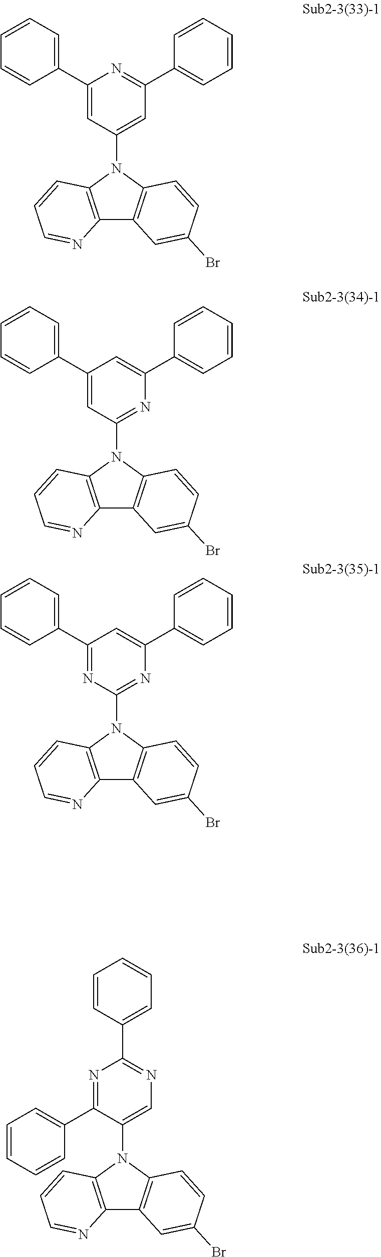

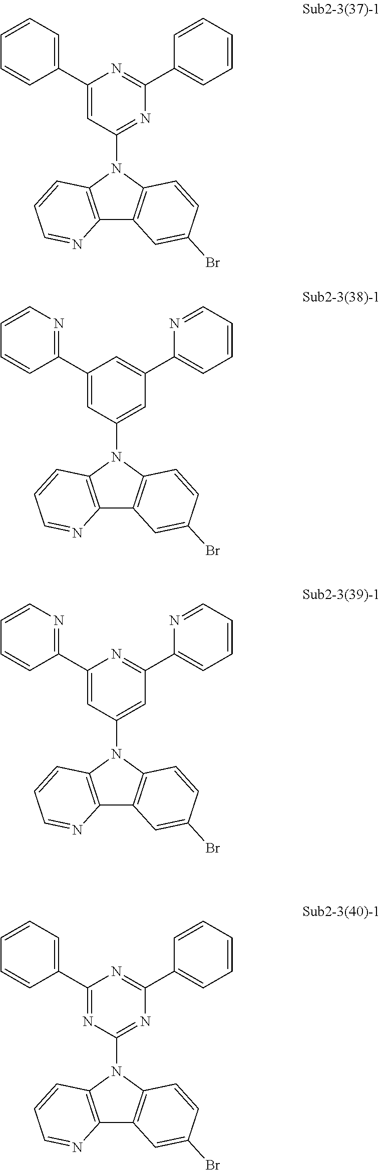

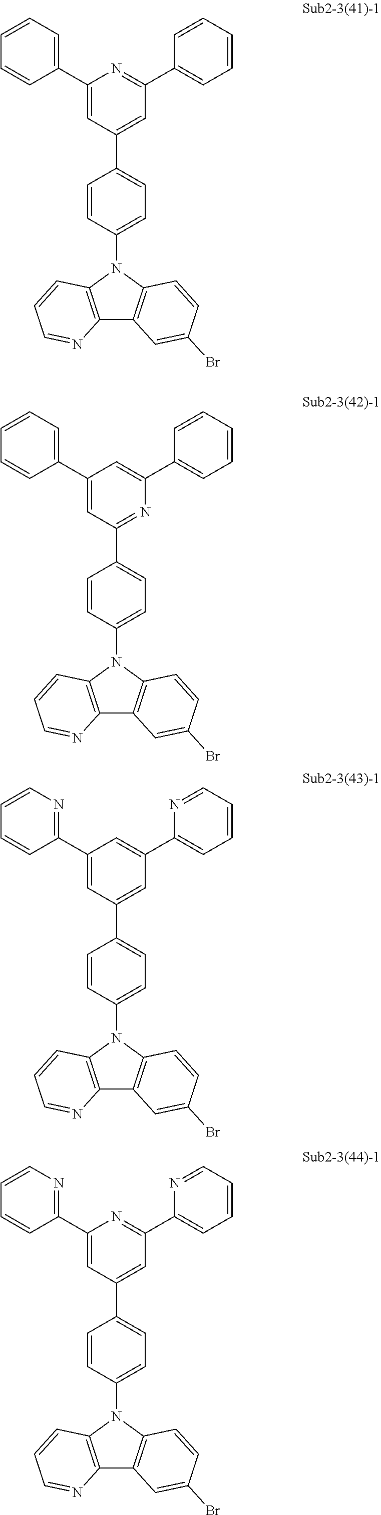

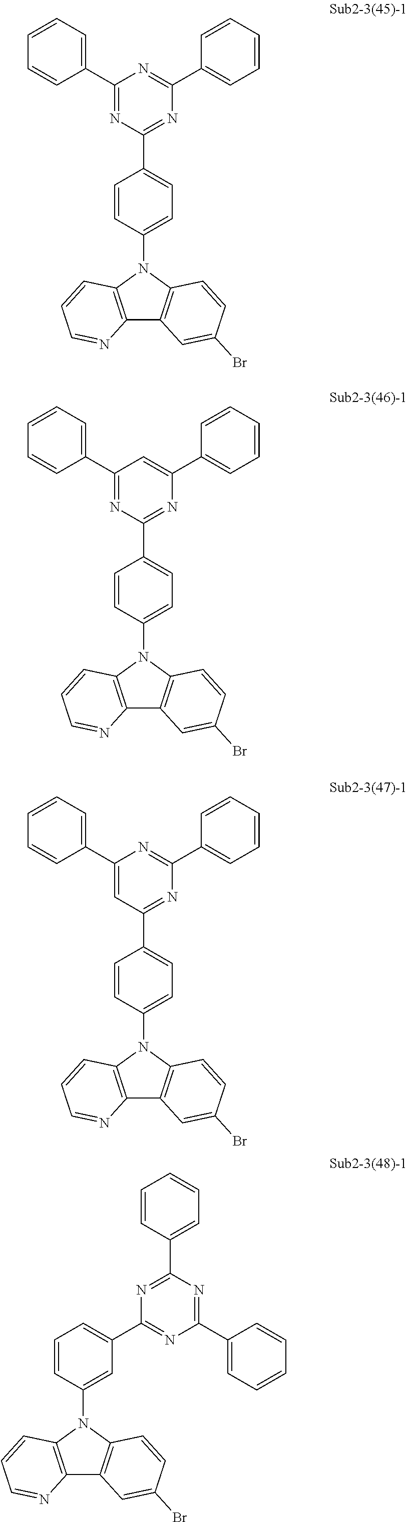

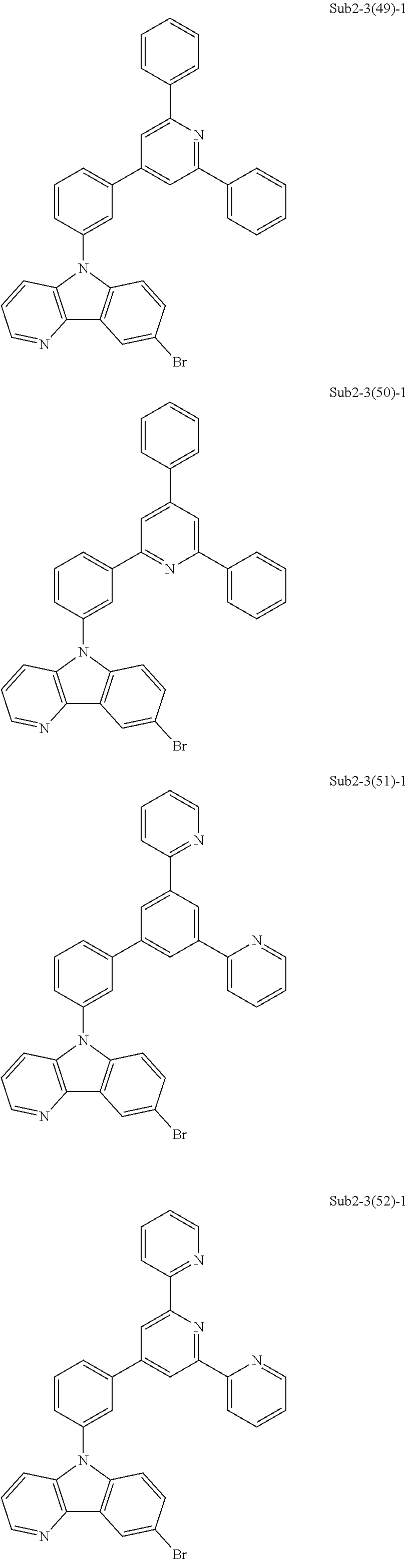

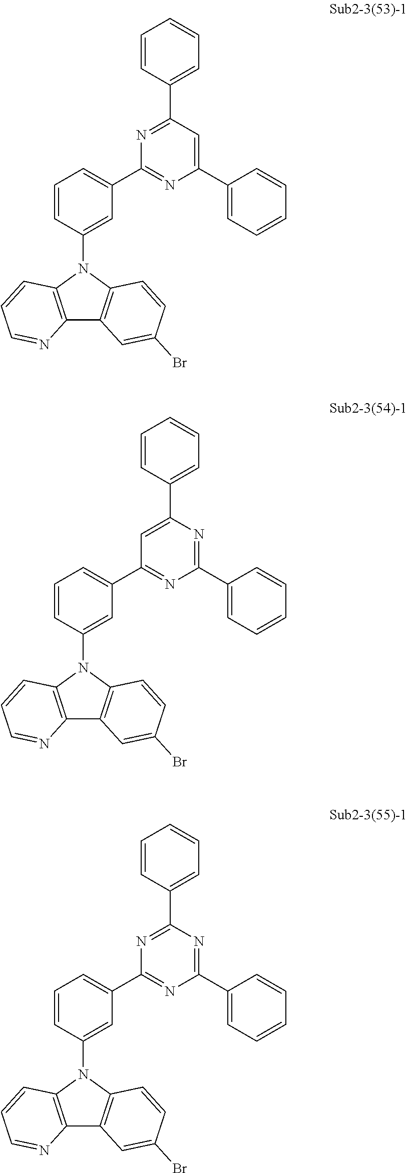 [SMILES: Brc1ccc2c(c1)c1ncccc1n2-c1cccc(-c2nc(-c3ccccc3)cc(-c3ccccc3)n2)c1 ; Brc1ccc2c(c1)c1ncccc1n2-c1cccc(-c2cc(-c3ccccc3)nc(-c3ccccc3)n2)c1 ; Brc1ccc2c(c1)c1ncccc1n2-c1cccc(-c2nc(-c3ccccc3)nc(-c3ccccc3)n2)c1]

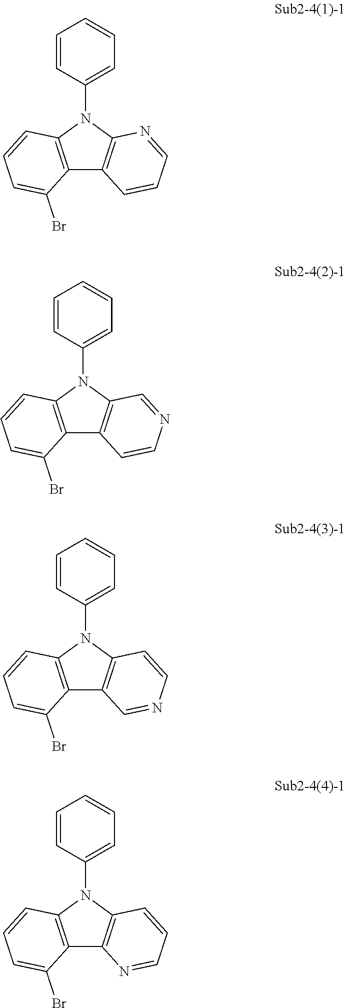 [SMILES: Brc1cccc2c1c1cccnc1n2-c1ccccc1 ; Brc1cccc2c1c1ccncc1n2-c1ccccc1 ; Brc1cccc2c1c1cnccc1n2-c1ccccc1 ; Brc1cccc2c1c1ncccc1n2-c1ccccc1]

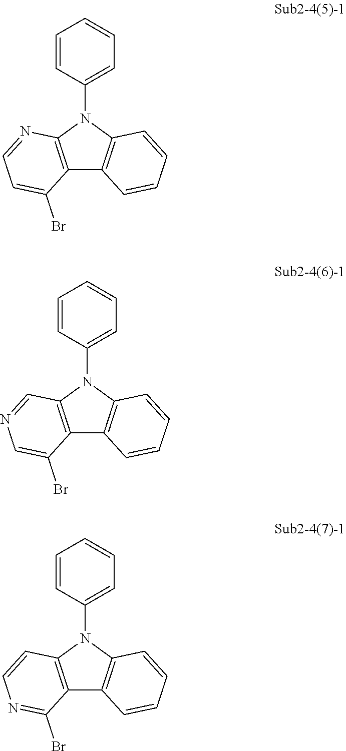 [SMILES: Brc1ccnc2c1c1ccccc1n2-c1ccccc1 ; Brc1cncc2c1c1ccccc1n2-c1ccccc1 ; Brc1nccc2c1c1ccccc1n2-c1ccccc1]

TABLE 1-3

| Compound | FD-MS | Compound | FD-MS |
|---|---|---|---|
| Sub2-1(1)-1 | m/z = 322.01(C₁₇H₁₁BrN₂ = 323.19) | Sub2-1(2)-1 | m/z = 322.01(C₁₇H₁₁BrN₂ = 323.19) |
| Sub2-1(3)-1 | m/z = 322.01(C₁₇H₁₁BrN₂ = 323.19) | Sub2-1(4)-1 | m/z = 322.01(C₁₇H₁₁BrN₂ = 323.19) |
| Sub2-1(5)-1 | m/z = 322.01(C₁₇H₁₁BrN₂ = 323.19) | Sub2-1(6)-1 | m/z = 322.01(C₁₇H₁₁BrN₂ = 323.19) |
| Sub2-1(7)-1 | m/z = 322.01(C₁₇H₁₁BrN₂ = 323.19) | Sub2-2(1)-1 | m/z = 322.01(C₁₇H₁₁BrN₂ = 323.19) |
| Sub2-2(2)-1 | m/z = 398.04(C₂₃H₁₅BrN₂ = 399.28) | Sub2-2(3)-1 | m/z = 398.04(C₂₃H₁₅BrN₂ = 399.28) |
| Sub2-2(4)-1 | m/z = 477.06(C₂₆H₁₆BrN₅ = 478.34) | Sub2-2(5)-1 | m/z = 475.07(C₂₈H₁₈BrN₃ = 476.37) |
| Sub2-2(6)-1 | m/z = 475.07(C₂₈H₁₈BrN₃ = 476.37) | Sub2-2(7)-1 | m/z = 476.06(C₂₈H₁₈BrN₃ = 477.35) |
| Sub2-2(8)-1 | m/z = 476.06(C₂₈H₁₈BrN₃ = 477.35) | Sub2-2(9)-1 | m/z = 476.06(C₂₈H₁₈BrN₃ = 477.35) |
| Sub2-2(10)-1 | m/z = 476.06(C₂ [symbol] H₁₈BrN₃ = 477.35) | Sub2-2(11)-1 | m/z = 477.06(C₂₆H₁₆BrN₅ = 478.34) |
| Sub2-2(12)-1 | m/z = 553.09(C₃₂H₂₀BrN₅ = 554.44) | Sub2-2(13)-1 | m/z = 552.09(C₃₃H₂₁BrN₄ = 553.45) |
| Sub2-2(14)-1 | m/z = 552.09(C₃₃H₂₁BrN₄ = 553.45) | Sub2-2(15)-1 | m/z = 551.10(C₃₄H₂₂BrN [symbol] = 552.46) |
| Sub2-2(16)-1 | m/z = 551.10(C₃₄H₂₂BrN₃ = 552.46) | Sub2-2(17)-1 | m/z = 552.09(C₃₃H₂₁BrN₄ = 553.45) |
| Sub2-2(18)-1 | m/z = 553.09(C₃₂H₂₀BrN₅ = 554.44) | Sub2-2(19)-1 | m/z = 553.09(C₃ [symbol] H₂₀BrN₅ = 554.44) |
| Sub2-2(20)-1 | m/z = 552.09(C₃₃H₂₁BrN₄ = 553.45) | Sub2-2(21)-1 | m/z = 552.09(C₃₃H₂₁BrN₄ = 553.45) |
| Sub2-2(22)-1 | m/z = 551.10(C₃₄H₂₂BrN₃ = 552.46) | Sub2-2(23)-1 | m/z = 551.10(C₃₄H₂₂BrN₃ = 552.46) |
| Sub2-2(24)-1 | m/z = 552.09(C₃₃H₂₁BrN₄ = 553.45) | Sub2-2(25)-1 | m/z = 552.09(C₃₃H₂₁BrN₄ = 553.45) |
| Sub2-2(26)-1 | m/z = 450.05(C₂₅H₁₅BrN₄ = 451.32) | Sub2-2(27)-1 | m/z = 322.01(C₁₇H₁₁BrN₂ = 323.19) |
| Sub2-2(28)-1 | m/z = 322.01(C₁₇H₁₁BrN₂ = 323.19) | Sub2-2(29)-1 | m/z = 322.01(C₁₇H₁₁BrN₂ = 323.19) |
| Sub2-2(30)-1 | m/z = 398.04(C₂₃H₁₅BrN₂ = 399.28) | Sub2-2(31)-1 | m/z = 398.04(C₂₃H₁₅BrN₂ = 399.28) |
| Sub2-2(32)-1 | m/z = 477.06(C₂₆H₁₆BrN₅ = 478.34) | Sub2-2(33)-1 | m/z = 476.06(C₂₈H₁₈BrN₃ = 477.35) |
| Sub2-2(34)-1 | m/z = 476.06(C₂₈H₁₈BrN₃ = 477.35) | Sub2-2(35)-1 | m/z = 476.06(C₂₈H₁₈BrN₃ = 477.35) |
| Sub2-2(36)-1 | m/z = 475.07(C₂₈H₁₈BrN₃ = 476.37) | Sub2-2(37)-1 | m/z = 475.07(C₂₈H₁₈BrN₃ = 476.37) |
| Sub2-2(38)-1 | m/z = 476.06(C₂₈H₁₈BrN₃ = 477.35) | Sub2-2(39)-1 | m/z = 476.06(C₂₈H₁₈BrN₃ = 477.35) |
| Sub2-2(40)-1 | m/z = 553.09(C₃₂H₂₀BrN₅ = 554.44) | Sub2-2(41)-1 | m/z = 552.09(C₃₃H₂₁BrN₄ = 553.45) |
| Sub2-2(42)-1 | m/z = 552.09(C₃₃H₂₁BrN₄ = 553.45) | Sub2-2(43)-1 | m/z = 551.10(C₃₄H₂₂BrN₃ = 552.46) |
| Sub2-2(44)-1 | m/z = 551.10(C₃₄H₂₂BrN₃ = 552.46) | Sub2-2(45)-1 | m/z = 552.09(C₃₃H₂₁BrN₄ = 553.45) |
| Sub2-2(46)-1 | m/z = 553.09(C₃₂H₂₀BrN₅ = 554.44) | Sub2-2(47)-1 | m/z = 553.09(C₃₂H₂₀BrN₅ = 554.44) |
| Sub2-2(48)-1 | m/z = 552.09(C₃₃H₂₁BrN₄ = 553.45) | Sub2-2(49)-1 | m/z = 552.09(C₃₃H₂₁BrN₄ = 553.45) |
| Sub2-2(50)-1 | m/z = 551.10(C₃₄H₂₂BrN₃ = 552.46) | Sub2-2(51)-1 | m/z = 551.10(C₃₄H₂₂BrN₃ = 552.46) |
| Sub2-2(52)-1 | m/z = 552.09(C₃₃H₂₁BrN₄ = 553.45) | Sub2-2(53)-1 | m/z = 553.09(C₃₂H₂₀BrN₅ = 554.44) |
| Sub2-2(54)-1 | m/z = 450.05(C₂₅H₁₅BrN₄ = 451.32) | Sub2-2(55)-1 | m/z = 322.01(C₁₇H₁₁BrN₂ = 323.19) |
| Sub2-2(56)-1 | m/z = 322.01(C₁₇H₁₁BrN₂ = 323.19) | Sub2-2(57)-1 | m/z = 322.01(C₁₇H₁₁BrN₂ = 323.19) |
| Sub2-3(1)-1 | m/z = 322.01(C₁₇H₁₁BrN₂ = 323.19) | Sub2-3(2)-1 | m/z = 398.04(C₂₃H₁₅BrN₂ = 399.28) |
| Sub2-3(3)-1 | m/z = 398.04(C₂₃H₁₅BrN₂ = 399.28) | Sub2-3(4)-1 | m/z = 477.06(C₂₆H₁₆BrN₅ = 478.34) |
| Sub2-3(5)-1 | m/z = 475.07(C₂₈H₁₈BrN₃ = 476.37) | Sub2-3(6)-1 | m/z = 475.07(C₂₈H₁₈BrN₃ = 476.37) |
| Sub2-3(7)-1 | m/z = 476.06(C₂₈H₁₈BrN₃ = 477.35) | Sub3-3(8)-1 | m/z = 476.06(C₂₈H₁₈BrN₃ = 477.35) |
| Sub2-3(9)-1 | m/z = 476.06(C₂₈H₁₈BrN₃ = 477.35) | Sub3-3(10)-1 | m/z = 476.06(C₂₈H₁₈BrN₃ = 477.35) |
| Sub2-3(11)-1 | m/z = 477.06(C₂₆H₁₆BrN₅ = 478.34) | Sub2-3(12)-1 | m/z = 551.10(C₃₄H₂₂BrN₃ = 552.46) |
| Sub2-3(13)-1 | m/z = 551.10(C₃₄H₂₂BrN₃ = 552.46) | Sub2-3(14)-1 | m/z = 552.09(C₃₃H₂₁BrN₄ = 553.45) |
| Sub2-3(15)-1 | m/z = 553.09(C₃₂H₂₀BrN₅ = 554.44) | Sub2-3(16)-1 | m/z = 553.09(C₃₂H₂₀BrN₅ = 554.44) |
| Sub2-3(17)-1 | m/z = 552.09(C₃₃H₂₁BrN₄ = 553.45) | Sub2-3(18)-1 | m/z = 552.09(C₃₃H₂₁BrN₄ = 553.45) |
| Sub2-3(19)-1 | m/z = 553.09(C₃₂H₂₀BrN₅ = 554.44) | Sub2-3(20)-1 | m/z = 552.09(C₃₃H₂₁BrN₄ = 553.45) |
| Sub2-3(21)-1 | m/z = 552.09(C₃₃H₂₁BrN₄ = 553.45) | Sub2-3(22)-1 | m/z = 551.10(C₃₄H₂₂BrN₃ = 552.46) |
| Sub2-3(23)-1 | m/z = 551.10(C₃₄H₂₂BrN₃ = 552.46) | Sub2-3(24)-1 | m/z = 552.09(C₃₃H₂₁BrN₄ = 553.45) |
| Sub2-3(25)-1 | m/z = 553.09(C₃₂H₂₀BrN₅ = 554.44) | Sub2-3(26)-1 | m/z = 450.05(C₂₅H₁₅BrN₄ = 451.32) |
| Sub2-3(27)-1 | m/z = 322.01(C₁₇H₁₁BrN₂ = 323.19) | Sub2-3(28)-1 | m/z = 322.01(C₁₇H₁₁BrN₂ = 399.28) |
| Sub2-3(29)-1 | m/z = 322.01(C₁₇H₁₁BrN₂ = 323.19) | Sub2-3(30)-1 | m/z = 398.04(C₂₃H₁₅BrN₂ = 399.28) |
| Sub2-3(31)-1 | m/z = 398.04(C₂₃H₁₅BrN₂ = 399.28) | Sub2-3(32)-1 | m/z = 450.05(C₂₅H₁₅BrN₄ = 451.32) |
| Sub2-3(33)-1 | m/z = 475.07(C₂₈H₁₈BrN₃ = 476.37) | Sub2-3(34)-1 | m/z = 475.07(C₂₈H₁₈BrN₃ = 476.37) |
| Sub2-3(35)-1 | m/z = 476.06(C₂₈H₁₈BrN₃ = 477.35) | Sub2-3(36)-1 | m/z = 476.06(C₂₈H₁₈BrN₃ = 477.35) |
| Sub2-3(37)-1 | m/z = 476.06(C₂₈H₁₈BrN₃ = 477.35) | Sub2-3(38)-1 | m/z = 476.06(C₂₈H₁₈BrN₃ = 477.35) |
| Sub2-3(39)-1 | m/z = 477.06(C₂₆H₁₆BrN₅ = 478.34) | Sub2-3(40)-1 | m/z = 477.06(C₂₆H₁₆BrN₅ = 478.34) |
| Sub2-3(41)-1 | m/z = 551.10(C₃₄H₂₂BrN₃ = 552.46) | Sub2-3(42)-1 | m/z = 551.10(C₃₄H₂₂BrN₃ = 552.46) |
| Sub2-3(43)-1 | m/z = 552.09(C₃₃H₂₁BrN₄ = 553.45) | Sub2-3(44)-1 | m/z = 553.09(C₃₂H₂₀BrN₅ = 554.44) |
| Sub2-3(45)-1 | m/z = 553.09(C₃₂H₂₀BrN₅ = 554.44) | Sub2-3(46)-1 | m/z = 552.09(C₃₃H₂₁BrN₄ = 553.45) |
| Sub2-3(47)-1 | m/z = 552.09(C₃₃H₂₁BrN₄ = 553.45) | Sub2-3(48)-1 | m/z = 553.09(C₃₂H₂₀BrN₅ = 554.44) |
| Sub2-3(49)-1 | m/z = 551.10(C₃₄H₂₂BrN₃ = 552.46) | Sub2-3(50)-1 | m/z = 551.10(C₃₄H₂₂BrN₃ = 552.46) |
| Sub2-3(51)-1 | m/z = 552.09(C₃₃H₂₁BrN₄ = 553.45) | Sub2-3(52)-1 | m/z = 553.09(C₃₂H₂₀BrN₅ = 554.44) |
| Sub2-3(53)-1 | m/z = 553.09(C₃₂H₂₀BrN₅ = 554.44) | Sub2-3(54)-1 | m/z = 552.09(C₃₃H₂₁BrN₄ = 553.45) |
| Sub2-3(55)-1 | m/z = 553.09(C₃₂H₂₀BrN₅ = 554.44) | Sub2-4(1)-1 | m/z = 322.01(C₁₇H₁₁BrN₂ = 323.19) |
| Sub2-4(2)-1 | m/z = 322.01(C₁₇H₁₁BrN₂ = 323.19) | Sub2-4(3)-1 | m/z = 322.01(C₁₇H₁₁BrN₂ = 323.19) |
| Sub2-4(4)-1 | m/z = 322.01(C₁₇H₁₁BrN₂ = 323.19) | Sub2-4(5)-1 | m/z = 322.01(C₁₇H₁₁BrN₂ = 323.19) |
| Sub2-4(6)-1 | m/z = 322.01(C₁₇H₁₁BrN₂ = 323.19) | Sub2-4(7)-1 | m/z = 322.01(C₁₇H₁₁BrN₂ = 323.19) |

[symbol]

indicates data missing or illegible when filed

### Synthesis Example of Final Products

In a round-bottom flask, compound Sub 1-1 (1 eq) was added, and then compound Sub 2-1 (1.1 eq), Pd(PPh₃)₄ (0.03-0.05 eq.), NaOH (3 eq), THF (3 mL/1 mmol), and water (1.5 mL/1 mmol) were added. Thereafter, the mixture was heated under reflux at 80-90□. Upon completion of the reaction, the reaction product was diluted with distilled water at room temperature, followed by extraction with methylene chloride and water. The organic layer was dried over MgSO₄ and concentrated, and then the generated compound was subjected to silica gel chromatography and recrystallization to give a product.

### Synthesis Example of Compound 1-1-1

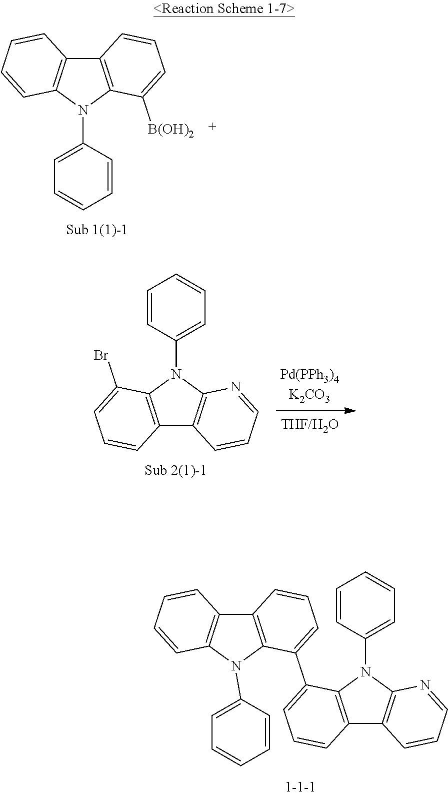 [SMILES: OB(O)c1cccc2c3ccccc3n(-c3ccccc3)c12 ; Brc1cccc2c3cccnc3n(-c3ccccc3)c12 ; c1ccc(-n2c3ccccc3c3cccc(-c4cccc5c6cccnc6n(-c6ccccc6)c45)c32)cc1]

In a round-bottom flask, (9-phenyl-9H-carbazol-1-yl)boronic acid (5.7 g, 20 mmol) was added, and 8-bromo-9-phenyl-9H-pyrido[2,3-b]indole (12.2 g, 22 mmol), Pd(PPh₃)₄ (0.5 g, 0.6 mmol), K₂CO₃ (8.3 g, 60 mmol), THF (60 mL), and water (30 mL) were added. Thereafter, the mixture was heated under reflux at 80-90□. Upon completion of the reaction, the reaction product was diluted with distilled water at room temperature, followed by extraction with methylene chloride and water. The organic layer was dried over MgSO₄ and concentrated, and then the thus generated compound was subjected to silica gel column chromatography and recrystallization to give a product 5.5 g (yield: 57%).

### 2. Synthesis Example of Compound 2-38-1

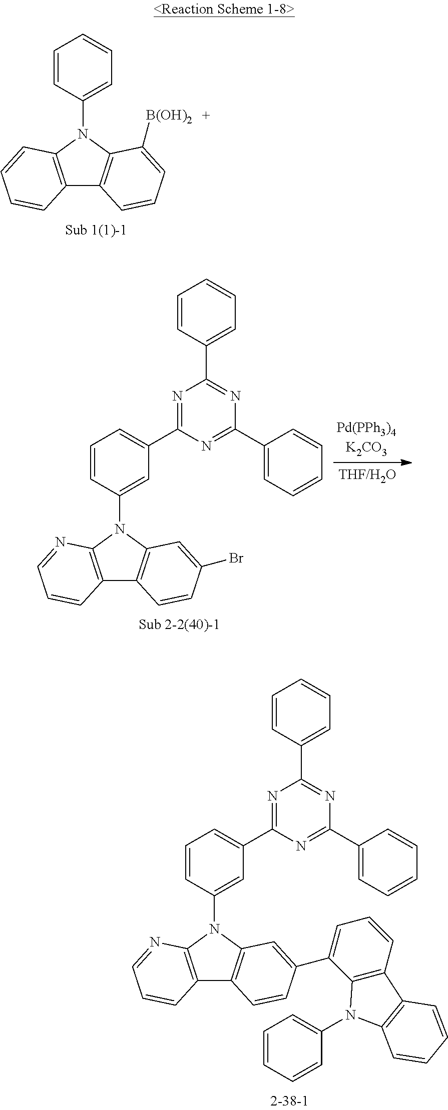 [SMILES: OB(O)c1cccc2c3ccccc3n(-c3ccccc3)c12 ; Brc1ccc2c3cccnc3n(-c3cccc(-c4nc(-c5ccccc5)nc(-c5ccccc5)n4)c3)c2c1 ; c1ccc(-c2nc(-c3ccccc3)nc(-c3cccc(-n4c5cc(-c6cccc7c8ccccc8n(-c8ccccc8)c67)ccc5c5cccnc54)c3)n2)cc1]

In a round-bottom flask, (9-phenyl-9H-carbazol-1-yl)boronic acid (5.7 g, 20 mmol) was added, and 7-bromo-9-(3-(4,6-diphenyl-1,3,5-triazin-2-yl)phenyl)-9H-pyrido[2,3-b]indole (12.2 g, 22 mmol), Pd(PPh₃)₄ (0.5 g, 0.6 mmol), K₂CO₃ (8.3 g, 60 mmol), THF (60 mL), and water (30 mL) were added. Thereafter, the mixture was heated under reflux at 80-90□. Upon completion of the reaction, the reaction product was diluted with distilled water at room temperature, followed by extraction with methylene chloride and water. The organic layer was dried over MgSO₄ and concentrated, and then the thus generated compound was subjected to silica gel column chromatography and recrystallization to give a product 8.2 g (yield: 57%).

### 3. Synthesis Example of Compound 2-70-1

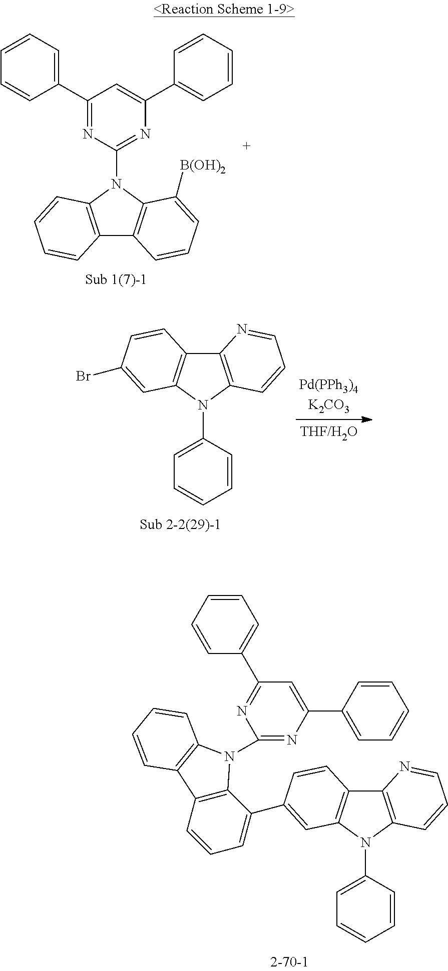 [SMILES: OB(O)c1cccc2c3ccccc3n(-c3nc(-c4ccccc4)cc(-c4ccccc4)n3)c12 ; Brc1ccc2c3ncccc3n(-c3ccccc3)c2c1 ; c1ccc(-c2cc(-c3ccccc3)nc(-n3c4ccccc4c4cccc(-c5ccc6c7ncccc7n(-c7ccccc7)c6c5)c43)n2)cc1]

In a round-bottom flask, (9-(4,6-diphenylpyrimidin-2-yl)-9H-carbazol-1-yl)boronic acid (8.8 g, 20 mmol) was added, and 7-bromo-9-(3-(4,6-diphenyl-1,3,5-triazin-2-yl)phenyl)-9H-pyrido[2,3-b]indole (12.2 g, 22 mmol), Pd(PPh₃)₄ (0.5 g, 0.6 mmol), K₂CO₃ (8.3 g, 60 mmol), THF (60 mL), and water (30 mL) were added. Thereafter, the mixture was heated under reflux at 80-90° C. Upon completion of the reaction, the reaction product was diluted with distilled water at room temperature, followed by extraction with methylene chloride and water. The organic layer was dried over MgSO₄ and concentrated, and then the thus generated compound was subjected to silica gel column chromatography and recrystallization to give a product 8.0 g (yield: 62%).

### 4. Synthesis Example of Compound 3-10-1

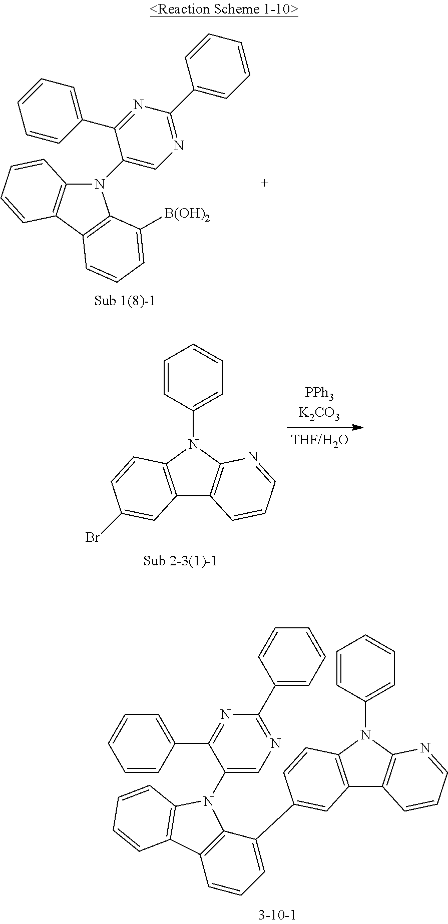 [SMILES: OB(O)c1cccc2c3ccccc3n(-c3cnc(-c4ccccc4)nc3-c3ccccc3)c12 ; Brc1ccc2c(c1)c1cccnc1n2-c1ccccc1 ; c1ccc(-c2ncc(-n3c4ccccc4c4cccc(-c5ccc6c(c5)c5cccnc5n6-c5ccccc5)c43)c(-c3ccccc3)n2)cc1]

In a round-bottom flask, (9-(2,4-diphenylpyrimidin-5-yl)-9H-carbazol-1-yl)boronic acid (8.8 g, 20 mmol) was added, and 6-bromo-9-phenyl-9H-pyrido[2,3-b]indole (7.1 g, 22 mmol), Pd(PPh₃)₄ (0.5 g, 0.6 mmol), K₂CO₃ (8.3 g, 60 mmol), THF (60 mL), and water (30 mL) were added. Thereafter, the mixture was heated under reflux at 80-90° C. Upon completion of the reaction, the reaction product was diluted with distilled water at room temperature, followed by extraction with methylene chloride and water. The organic layer was dried over MgSO₄ and concentrated, and then the thus generated compound was subjected to silica gel column chromatography and recrystallization to give a product 7.3 g (yield: 57%).

### 5. Synthesis Example of Compound 3-68-1

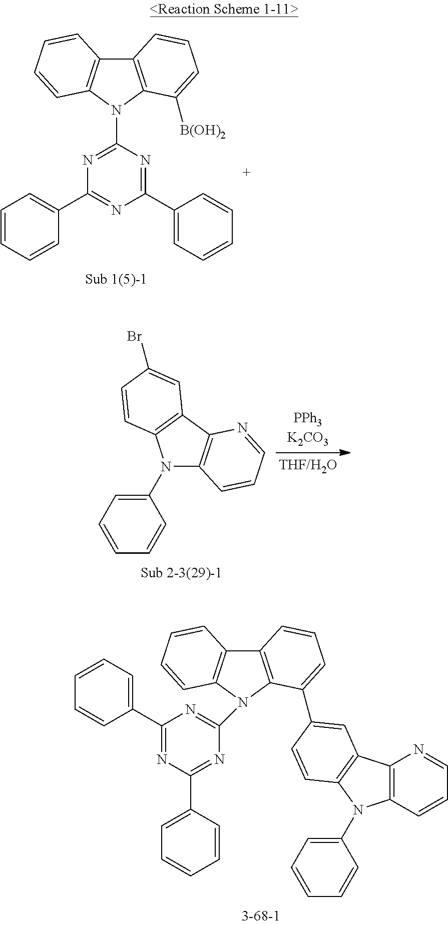 [SMILES: OB(O)c1cccc2c3ccccc3n(-c3nc(-c4ccccc4)nc(-c4ccccc4)n3)c12 ; Brc1ccc2c(c1)c1ncccc1n2-c1ccccc1 ; c1ccc(-c2nc(-c3ccccc3)nc(-n3c4ccccc4c4cccc(-c5ccc6c(c5)c5ncccc5n6-c5ccccc5)c43)n2)cc1]

In a round-bottom flask, (9-(4,6-diphenyl-1,3,5-triazin-2-yl)-9H-carbazol-1-yl)boronic acid (8.8 g, 20 mmol) was added, and 8-bromo-5-phenyl-5H-pyrido[3,2-b]indole (7.1 g, 22 mmol), Pd(PPh₃)₄ (0.5 g, 0.6 mmol), K₂CO₃ (8.3 g, 60 mmol), THF (60 mL), and water (30 mL) were added. Thereafter, the mixture was heated under reflux at 80-90° C. Upon completion of the reaction, the reaction product was diluted with distilled water at room temperature, followed by extraction with methylene chloride and water. The organic layer was dried over MgSO₄ and concentrated, and then the thus generated compound was subjected to silica gel column chromatography and recrystallization to give a product 7.0 g (yield: 54%).

### 6. Synthesis Example of Compound 3-76-1

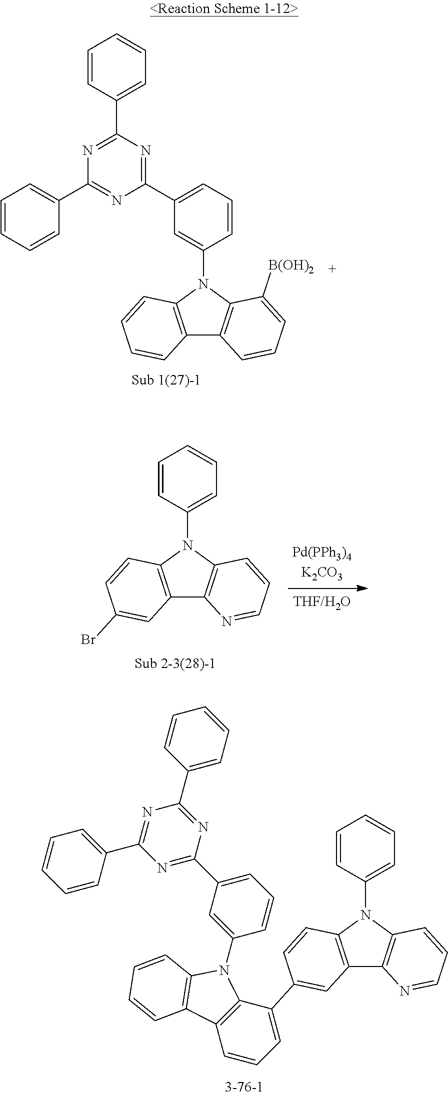 [SMILES: OB(O)c1cccc2c3ccccc3n(-c3cccc(-c4nc(-c5ccccc5)nc(-c5ccccc5)n4)c3)c12 ; Brc1ccc2c(c1)c1ncccc1n2-c1ccccc1 ; c1ccc(-c2nc(-c3ccccc3)nc(-c3cccc(-n4c5ccccc5c5cccc(-c6ccc7c(c6)c6ncccc6n7-c6ccccc6)c54)c3)n2)cc1]

In a round-bottom flask, (9-(3-(4,6-diphenyl-1,3,5-triazin-2-yl)phenyl)-9H-carbazol-1-yl)boronic acid (10.4 g, 20 mmol) was added, and 8-bromo-5-phenyl-5H-pyrido[3,2-b]indole (7.1 g, 22 mmol), Pd(PPh₃)₄ (0.5 g, 0.6 mmol), K₂CO₃ (8.3 g, 60 mmol), THF (60 mL), water (30 mL) were added. Thereafter, the mixture was heated under reflux at 80-90° C. Upon completion of the reaction, the reaction product was diluted with distilled water at room temperature, followed by extraction with methylene chloride and water. The organic layer was dried over MgSO₄ and concentrated, and then the thus generated compound was subjected to silica gel column chromatography and recrystallization to give a product 10.5 g (yield: 73%).

### 7. Synthesis Example of Compound 4-23-1

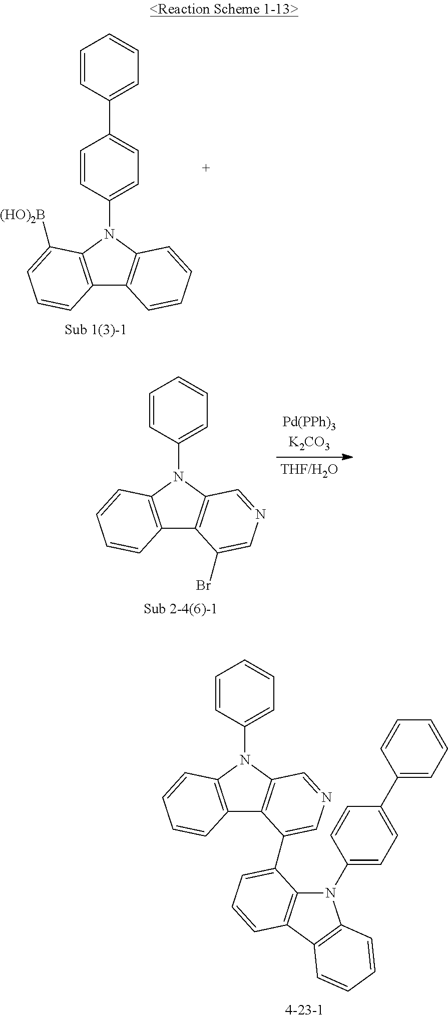 [SMILES: OB(O)c1cccc2c3ccccc3n(-c3ccc(-c4ccccc4)cc3)c12 ; Brc1cncc2c1c1ccccc1n2-c1ccccc1 ; c1ccc(-c2ccc(-n3c4ccccc4c4cccc(-c5cncc6c5c5ccccc5n6-c5ccccc5)c43)cc2)cc1]

In a round-bottom flask, (9-([1,1′-biphenyl]-4-yl)-9H-carbazol-1-yl)boronic acid (7.2 g, 20 mmol) was added, and 4-bromo-9-phenyl-9H-pyrido[3,4-b]indole (7.1 g, 22 mmol), Pd(PPh₃)₄ (0.5 g, 0.6 mmol), K₂CO₃ (8.3 g, 60 mmol), THF (60 mL), and water (30 mL) were added. Thereafter, the mixture was heated under reflux at 80-90° C. Upon completion of the reaction, the reaction product was diluted with distilled water at room temperature, followed by extraction with methylene chloride and water. The organic layer was dried over MgSO₄ and concentrated, and then the thus generated compound was subjected to silica gel column chromatography and recrystallization to give a product 7.8 g (yield: 69%).

Meanwhile, FD-MS values of compounds 1-1-1 to 1-28-1, 2-1-1 to 2-128-1, 3-1-1 to 3-128-1, 4-1-1 to 4-28-1, and 5-1-1 to 5-4-1 of the present invention prepared by the above synthesis examples are shown as in table 1-4 below.

TABLE 1-4

| Compound | FD-MS | Compound | FD-MS |
|---|---|---|---|
| 1-1-1 | m/z = 485.19 (C₃₅H₂₃N₃ = 485.58) | 1-2-1 | m/z = 535.20 (C₃₉H₂₅N₃ = 535.64) |
| 1-3-1 | m/z = 561.22 (C₄₁H₂₇N₃ = 561.67) | 1-4-1 | m/z = 640.24 (C₄₄H₂₈N₆ = 640.73) |
| 1-5-1 | m/z = 485.19 (C₃₅H₂₃N₃ = 485.58) | 1-6-1 | m/z = 535.20 (C₃₉H₂₅N₃ = 535.64) |
| 1-7-1 | m/z = 561.22 (C₄₁H₂₇N₃ = 561.67) | 1-8-1 | m/z = 640.24 (C₄₄H₂₈N₆ = 640.73) |
| 1-9-1 | m/z = 485.19 (C₃₅H₂₃N₃ = 485.58) | 1-10-1 | m/z = 535.20 (C₃₉H₂₅N₃ = 535.64) |
| 1-11-1 | m/z = 561.22 (C₄₁H₂₇N₃ = 561.67) | 1-12-1 | m/z = 640.24 (C₄₄H₂₈N₆ = 640.73) |
| 1-13-1 | m/z = 485.19 (C₃₅H₂₃N₃ = 485.58) | 1-14-1 | m/z = 535.20 (C₃₉H₂₅N₃ = 535.64) |
| 1-15-1 | m/z = 561.22 (C₄₁H₂₇N₃ = 561.67) | 1-16-1 | m/z = 640.24 (C₄₄H₂₈N₆ = 640.73) |
| 1-17-1 | m/z = 485.19 (C₃₅H₂₃N₃ = 485.58) | 1-18-1 | m/z = 535.20 (C₃₉H₂₅N₃ = 535.64) |
| 1-19-1 | m/z = 561.22 (C₄₁H₂₇N₃ = 561.67) | 1-20-1 | m/z = 640.24 (C₄₄H₂₈N₆ = 640.73) |
| 1-21-1 | m/z = 485.19 (C₃₅H₂₃N₃ = 485.58) | 1-22-1 | m/z = 535.20 (C₃₉H₂₅N₃ = 535.64) |
| 1-23-1 | m/z = 561.22 (C₄₁H₂₇N₃ = 561.67) | 1-24-1 | m/z = 640.24 (C₄₄H₂₈N₆ = 640.73) |
| 1-25-1 | m/z = 485.19 (C₃₅H₂₃N₃ = 485.58) | 1-26-1 | m/z = 535.20 (C₃₉H₂₅N₃ = 535.64) |
| 1-27-1 | m/z = 561.22 (C₄₁H₂₇N₃ = 561.67) | 1-28-1 | m/z = 640.24 (C₄₄H₂₅N₆ = 640.73) |
| 2-1-1 | m/z = 485.19 (C₃₅H₂₃N₃ = 485.58) | 2-2-1 | m/z = 561.22 (C₄₁H₂₇N₃ = 561.67) |
| 2-3-1 | m/z = 561.22 (C₄₁H₂₇N₃ = 561.67) | 2-4-1 | m/z = 637.25 (C₄₇H₃₁N₃ = 637.77) |
| 2-5-1 | m/z = 637.25 (C₄₇H₃₁N₃ = 637.77) | 2-6-1 | m/z = 637.25 (C₄₇H₃₁N₃ = 637.77) |
| 2-7-1 | m/z = 637.25 (C₄₇H₃₁N₃ = 637.77) | 2-8-1 | m/z = 639.24 (C₄₅H₂₉N₅ = 639.75) |
| 2-9-1 | m/z = 639.24 (C₄₅H₂₉N₅ = 639.75) | 2-10-1 | m/z = 639.24 (C₄₅H₂₉N₅ = 639.75) |
| 2-11-1 | m/z = 638.25 (C₄₆H₃₀N₄ = 638.76) | 2-12-1 | m/z = 638.25 (C₄₆H₃₀N₄ = 638.76) |
| 2-13-1 | m/z = 639.24 (C₄₅H₂₉N₅ = 639.75) | 2-14-1 | m/z = 640.24 (C₄₄H₂₈N₆ = 640.73) |
| 2-15-1 | m/z = 716.27 (C₅₀H₃₂N₆ = 716.83) | 2-16-1 | m/z = 715.27 (C₅₁H₃₃N₅ = 715.84) |
| 2-17-1 | m/z = 715.27 (C₅₁H₃₃N₅ = 715.84) | 2-18-1 | m/z = 714.28 (C₅₂H₃₄N₄ = 714.85) |
| 2-19-1 | m/z = 714.28 (C₅₂H₃₄N₄ = 714.85) | 2-20-1 | m/z = 715.27 (C₅₁H₃₃N₅ = 715.84) |
| 2-21-1 | m/z = 716.27 (C₅₀H₃₂N₆ = 716.83) | 2-22-1 | m/z = 716.27 (C₅₀H₃₂N₆ = 716.83) |
| 2-23-1 | m/z = 715.27 (C₅₁H₃₃N₅ = 715.84) | 2-24-1 | m/z = 715.27 (C₅₁H₃₃N₅ = 715.84) |
| 2-25-1 | m/z = 714.28 (C₅₂H₃₄N₄ = 714.85) | 2-26-1 | m/z = 714.28 (C₅₂H₃₄N₄ = 714.85) |
| 2-27-1 | m/z = 715.27 (C₅₁H₃₃N₅ = 715.84) | 2-28-1 | m/z = 716.27 (C₅₀H₃₂N₆ = 716.83) |
| 2-29-1 | m/z = 613.23 (C₄₃H₂₇N₅ = 613.71) | 2-30-1 | m/z = 640.24 (C₄₄H₂₈N₆ = 640.73) |
| 2-31-1 | m/z = 639.24 (C₄₅H₂₉N₅ = 639.75) | 2-32-1 | m/z = 639.24 (C₄₅H₂₉N₅ = 639.75) |
| 2-33-1 | m/z = 639.24 (C₄₅H₂₉N₅ = 639.75) | 2-34-1 | m/z = 638.25 (C₄₆H₃₀N₄ = 638.76) |
| 2-35-1 | m/z = 638.25 (C₄₆H₃₀N₄ = 638.76) | 2-36-1 | m/z = 639.24 (C₄₅H₂₉N₅ = 639.75) |
| 2-37-1 | m/z = 640.24 (C₄₄H₂₈N₆ = 640.73) | 2-38-1 | m/z = 716.27 (C₅₀H₃₂N₆ = 716.83) |
| 2-39-1 | m/z = 715.27 (C₅₁H₃₃N₅ = 715.84) | 2-40-1 | m/z = 715.27 (C₅₁H₃₃N₅ = 715.84) |
| 2-41-1 | m/z = 714.28 (C₅₂H₃₄N₄ = 714.85) | 2-42-1 | m/z = 714.28 (C₅₂H₃₄N₄ = 714.85) |
| 2-43-1 | m/z = 715.27 (C₅₁H₃₃N₅ = 715.84) | 2-44-1 | m/z = 716.27 (C₅₀H₃₂N₆ = 716.83) |
| 2-45-1 | m/z = 716.27 (C₅₀H₃₂N₆ = 716.83) | 2-46-1 | m/z = 715.27 (C₅₁H₃₃N₅ = 715.84) |
| 2-47-1 | m/z = 715.27 (C₅₁H₃₃N₅ = 715.84) | 2-48-1 | m/z = 714.28 (C₅₂H₃₄N₄ = 714.85) |
| 2-49-1 | m/z = 714.28 (C₅₂H₃₄N₄ = 714.85) | 2-50-1 | m/z = 715.27 (C₅₁H₃₃N₅ = 715.84) |
| 2-51-1 | m/z = 716.27 (C₅₀H₃₂N₆ = 716.83) | 2-52-1 | m/z = 613.23 (C₄₃H₂₇N₅ = 613.71) |
| 2-53-1 | m/z = 485.19 (C₃₅H₂₃N₃ = 485.58) | 2-54-1 | m/z = 535.20 (C₃₉H₂₅N₃ = 535.64) |
| 2-55-1 | m/z = 561.22 (C₄₁H₂₇N₃ = 561.67) | 2-56-1 | m/z = 640.24 (C₄₄H₂₈N₆ = 640.73) |
| 2-57-1 | m/z = 485.19 (C₃₅H₂₃N₃ = 485.58) | 2-58-1 | m/z = 535.20 (C₃₉H₂₅N₃ = 535.64) |
| 2-59-1 | m/z = 561.22 (C₄₁H₂₇N₃ = 561.67) | 2-60-1 | m/z = 640.24 (C₄₄H₂₈N₆ = 640.73) |
| 2-61-1 | m/z = 485.19 (C₃₅H₂₃N₃ = 485.58) | 2-62-1 | m/z = 561.22 (C₄₁H₂₇N₃ = 561.67) |
| 2-63-1 | m/z = 561.22 (C₄₁H₂₇N₃ = 561.67) | 2-64-1 | m/z = 637.25 (C₄₇H₃₁N₃ = 637.77) |
| 2-65-1 | m/z = 637.25 (C₄₇H₃₁N₃ = 637.77) | 2-66-1 | m/z = 637.25 (C₄₇H₃₁N₃ = 637.77) |
| 2-67-1 | m/z = 637.25 (C₄₇H₃₁N₃ = 637.77) | 2-68-1 | m/z = 640.24 (C₄₄H₂₈N₆ = 640.73) |
| 2-69-1 | m/z = 639.24 (C₄₅H₂₉N₅ = 639.75) | 2-70-1 | m/z = 639.24 (C₄₅H₂₉N₅ = 639.75) |
| 2-71-1 | m/z = 639.24 (C₄₅H₂₉N₅ = 639.75) | 2-72-1 | m/z = 638.25 (C₄₆H₃₀N₄ = 638.76) |
| 2-73-1 | m/z = 638.25 (C₄₆H₃₀N₄ = 638.76) | 2-74-1 | m/z = 639.24 (C₄₅H₂₉N₅ = 639.75) |
| 2-75-1 | m/z = 640.24 (C₄₄H₂₈N₆ = 640.73) | 2-76-1 | m/z = 716.27 (C₅₀H₃₂N₆ = 716.83) |
| 2-77-1 | m/z = 715.27 (C₅₁H₃₃N₅ = 715.84) | 2-78-1 | m/z = 715.27 (C₅₁H₃₃N₅ = 715.84) |
| 2-79-1 | m/z = 714.28 (C₅₂H₃₄N₄ = 714.85) | 2-80-1 | m/z = 714.28 (C₅₂H₃₄N₄ = 714.85) |
| 2-81-1 | m/z = 715.27 (C₅₁H₃₃N₅ = 715.84) | 2-82-1 | m/z = 716.27 (C₅₀H₃₂N₆ = 716.83) |
| 2-83-1 | m/z = 716.27 (C₅₀H₃₂N₆ = 716.83) | 2-84-1 | m/z = 715.27 (C₅₁H₃₃N₅ = 715.84) |
| 2-85-1 | m/z = 715.27 (C₅₁H₃₃N₅ = 715.84) | 2-86-1 | m/z = 714.28 (C₅₂H₃₄N₄ = 714.85) |
| 2-87-1 | m/z = 714.28 (C₅₂H₃₄N₄ = 714.85) | 2-88-1 | m/z = 715.27 (C₅₁H₃₃N₅ = 715.84) |
| 2-89-1 | m/z = 716.27 (C₅₀H₃₂N₆ = 716.83) | 2-90-1 | m/z = 613.23 (C₄₃H₂₇N₅ = 613.71) |
| 2-91-1 | m/z = 640.24 (C₄₄H₂₈N₆ = 640.73) | 2-92-1 | m/z = 639.24 (C₄₅H₂₉N₅ = 639.75) |
| 2-93-1 | m/z = 639.24 (C₄₅H₂₉N₅ = 639.75) | 2-94-1 | m/z = 639.24 (C₄₅H₂₉N₅ = 639.75) |
| 2-95-1 | m/z = 638.25 (C₄₆H₃₀N₄ = 638.76) | 2-96-1 | m/z = 638.25 (C₄₆H₃₀N₄ = 638.76) |
| 2-97-1 | m/z = 639.24 (C₄₅H₂₉N₅ = 639.75) | 2-98-1 | m/z = 640.24 (C₄₄H₂₈N₆ = 640.73) |
| 2-99-1 | m/z = 716.27 (C₅₀H₃₂N₆ = 716.83) | 2-100-1 | m/z = 715.27 (C₅₁H₃₃N₅ = 715.84) |
| 2-101-1 | m/z = 715.27 (C₅₁H₃₃N₅ = 715.84) | 2-102-1 | m/z = 714.28 (C₅₂H₃₄N₄ = 714.85) |
| 2-103-1 | m/z = 714.28 (C₅₂H₃₄N₄ = 714.85) | 2-104-1 | m/z = 715.27 (C₅₁H₃₃N₅ = 715.84) |
| 2-105-1 | m/z = 716.27 (C₅₀H₃₂N₆ = 716.83) | 2-106-1 | m/z = 716.27 (C₅₀H₃₂N₆ = 716.83) |
| 2-107-1 | m/z = 715.27 (C₅₁H₃₃N₅ = 715.84) | 2-108-1 | m/z = 715.27 (C₅₁H₃₃N₅ = 715.84) |
| 2-109-1 | m/z = 714.28 (C₅₂H₃₄N₄ = 714.85) | 2-110-1 | m/z = 714.28 (C₅₂H₃₄N₄ = 714.85) |
| 2-111-1 | m/z = 715.27 (C₅₁H₃₃N₅ = 715.84) | 2-112-1 | m/z = 716.27 (C₅₀H₃₂N₆ = 716.83) |
| 2-113-1 | m/z = 613.23 (C₄₃H₂₇N₅ = 613.71) | 2-114-1 | m/z = 485.19 (C₃₅H₂₃N₃ = 485.58) |
| 2-115-1 | m/z = 535.20 (C₃₉H₂₅N₃ = 535.64) | 2-116-1 | m/z = 561.22 (C₄₁H₂₇N₃ = 561.67) |
| 2-117-1 | m/z = 640.24 (C₄₄H₂₈N₆ = 640.73) | 2-118-1 | m/z = 485.19 (C₃₅H₂₃N₃ = 485.58) |
| 2-119-1 | m/z = 535.20 (C₃₉H₂₅N₃ = 535.64) | 2-120-1 | m/z = 561.22 (C₄₁H₂₇N₃ = 561.67) |
| 2-121-1 | m/z = 640.24 (C₄₄H₂₈N₆ = 640.73) | 2-122-1 | m/z = 485.19 (C₃₅H₂₃N₃ = 485.58) |
| 2-123-1 | m/z = 535.20 (C₃₉H₂₅N₃ = 535.64) | 2-124-1 | m/z = 561.22 (C₄₁H₂₇N₃ = 561.67) |
| 2-125-1 | m/z = 640.24 (C₄₄H₂₈N₆ = 640.73) | 2-126-1 | m/z = 640.24 (C₄₄H₂₈N₆ = 640.73) |
| 2-127-1 | m/z = 535.20 (C₃₉H₂₅N₃ = 535.64) | 2-128-1 | m/z = 535.20 (C₃₉H₂₅N₃ = 535.64) |
| 3-1-1 | m/z = 485.19 (C₃₅H₂₃N₃ = 485.58) | 3-2-1 | m/z = 561.22 (C₄₁H₂₇N₃ = 561.67) |
| 3-3-1 | m/z = 561.22 (C₄₁H₂₇N₃ = 561.67) | 3-4-1 | m/z = 637.25 (C₄₇H₃₁N₃ = 637.77) |
| 3-5-1 | m/z = 637.25 (C₄₇H₃₁N₃ = 637.77) | 3-6-1 | m/z = 637.25 (C₄₇H₃₁N₃ = 637.77) |
| 3-7-1 | m/z = 637.25 (C₄₇H₃₁N₃ = 637.77) | 3-8-1 | m/z = 639.24 (C₄₅H₂₉N₅ = 639.75) |
| 3-9-1 | m/z = 639.24 (C₄₅H₂₉N₅ = 639.75) | 3-10-1 | m/z = 639.24 (C₄₅H₂₉N₅ = 639.75) |
| 3-11-1 | m/z = 638.25 (C₄₆H₃₀N₄ = 638.76) | 3-12-1 | m/z = 638.25 (C₄₆H₃₀N₄ = 638.76) |
| 3-13-1 | m/z = 639.24 (C₄₅H₂₉N₅ = 639.75) | 3-14-1 | m/z = 640.24 (C₄₄H₂₈N₆ = 640.73) |
| 3-15-1 | m/z = 716.27 (C₅₀H₃₂N₆ = 716.83) | 3-16-1 | m/z = 715.27 (C₅₁H₃₃N₅ = 715.84) |
| 3-17-1 | m/z = 715.27 (C₅₁H₃₃N₅ = 715.84) | 3-18-1 | m/z = 714.28 (C₅₂H₃₄N₄ = 714.85) |
| 3-19-1 | m/z = 714.28 (C₅₂H₃₄N₄ = 714.85) | 3-20-1 | m/z = 715.27 (C₅₁H₃₃N₅ = 715.84) |
| 3-21-1 | m/z = 716.27 (C₅₀H₃₂N₆ = 716.83) | 3-22-1 | m/z = 716.27 (C₅₀H₃₂N₆ = 716.83) |
| 3-23-1 | m/z = 715.27 (C₅₁H₃₃N₅ = 715.84) | 3-24-1 | m/z = 715.27 (C₅₁H₃₃N₅ = 715.84) |
| 3-25-1 | m/z = 714.28 (C₅₂H₃₄N₄ = 714.85) | 3-26-1 | m/z = 714.28 (C₅₂H₃₄N₄ = 714.85) |
| 3-27-1 | m/z = 715.27 (C₅₁H₃₃N₅ = 715.84) | 3-28-1 | m/z = 716.27 (C₅₀H₃₂N₆ = 716.83) |
| 3-29-1 | m/z = 613.23 (C₄₃H₂₇N₅ = 613.71) | 3-30-1 | m/z = 640.24 (C₄₄H₂₈N₆ = 640.73) |
| 3-31-1 | m/z = 639.24 (C₄₅H₂₉N₅ = 639.75) | 3-32-1 | m/z = 639.24 (C₄₅H₂₉N₅ = 639.75) |
| 3-33-1 | m/z = 639.24 (C₄₅H₂₉N₅ = 639.75) | 3-34-1 | m/z = 638.25 (C₄₆H₃₀N₄ = 638.76) |
| 3-35-1 | m/z = 638.25 (C₄₆H₃₀N₄ = 638.76) | 3-36-1 | m/z = 639.24 (C₄₅H₂₉N₅ = 639.75) |
| 3-37-1 | m/z = 640.24 (C₄₄H₂₈N₆ = 640.73) | 3-38-1 | m/z = 716.27 (C₅₀H₃₂N₆ = 716.83) |
| 3-39-1 | m/z = 715.27 (C₅₁H₃₃N₅ = 715.84) | 3-40-1 | m/z = 715.27 (C₅₁H₃₃N₅ = 715.84) |
| 3-41-1 | m/z = 714.28 (C₅₂H₃₄N₄ = 714.85) | 3-42-1 | m/z = 714.28 (C₅₂H₃₄N₄ = 714.85) |
| 3-43-1 | m/z = 715.27 (C₅₁H₃₃N₅ = 715.84) | 3-44-1 | m/z = 716.27 (C₅₀H₃₂N₆ = 716.83) |
| 3-45-1 | m/z = 716.27 (C₅₀H₃₂N₆ = 716.83) | 3-46-1 | m/z = 715.27 (C₅₁H₃₃N₅ = 715.84) |
| 3-47-1 | m/z = 715.27 (C₅₁H₃₃N₅ = 715.84) | 3-48-1 | m/z = 714.28 (C₅₂H₃₄N₄ = 714.85) |
| 3-49-1 | m/z = 714.28 (C₅₂H₃₄N₄ = 714.85) | 3-50-1 | m/z = 715.27 (C₅₁H₃₃N₅ = 715.84) |
| 3-51-1 | m/z = 716.27 (C₅₀H₃₂N₆ = 716.83) | 3-52-1 | m/z = 613.23 (C₄₃H₂₇N₅ = 613.71) |
| 3-53-1 | m/z = 485.19 (C₃₅H₂₃N₃ = 485.58) | 3-54-1 | m/z = 535.20 (C₃₉H₂₅N₃ = 535.64) |
| 3-55-1 | m/z = 561.22 (C₄₁H₂₇N₃ = 561.67) | 3-56-1 | m/z = 640.24 (C₄₄H₂₈N₆ = 640.73) |
| 3-57-1 | m/z = 485.19 (C₃₅H₂₃N₃ = 485.58) | 3-58-1 | m/z = 535.20 (C₃₉H₂₅N₃ = 535.64) |
| 3-59-1 | m/z = 561.22 (C₄₁H₂₇N₃ = 561.67) | 3-60-1 | m/z = 640.24 (C₄₄H₂₈N₆ = 640.73) |
| 3-61-1 | m/z = 485.19 (C₃₅H₂₃N₃ = 485.58) | 3-62-1 | m/z = 561.22 (C₄₁H₂₇N₃ = 561.67) |
| 3-63-1 | m/z = 561.22 (C₄₁H₂₇N₃ = 561.67) | 3-64-1 | m/z = 637.25 (C₄₇H₃₁N₃ = 637.77) |
| 3-65-1 | m/z = 637.25 (C₄₇H₃₁N₃ = 637.77) | 3-66-1 | m/z = 637.25 (C₄₇H₃₁N₃ = 637.77) |
| 3-67-1 | m/z = 637.25 (C₄₇H₃₁N₃ = 637.77) | 3-68-1 | m/z = 640.24 (C₄₄H₂₈N₆ = 640.73) |
| 3-69-1 | m/z = 639.24 (C₄₅H₂₉N₅ = 639.75) | 3-70-1 | m/z = 639.24 (C₄₅H₂₉N₅ = 639.75) |
| 3-71-1 | m/z = 639.24 (C₄₅H₂₉N₅ = 639.75) | 3-72-1 | m/z = 638.25 (C₄₆H₃₀N₄ = 638.76) |
| 3-73-1 | m/z = 638.25 (C₄₆H₃₀N₄ = 638.76) | 3-74-1 | m/z = 639.24 (C₄₅H₂₉N₅ = 639.75) |
| 3-75-1 | m/z = 640.24 (C₄₄H₂₈N₆ = 640.73) | 3-76-1 | m/z = 716.27 (C₅₀H₃₂N₆ = 716.83) |
| 3-77-1 | m/z = 715.27 (C₅₁H₃₃N₅ = 715.84) | 3-78-1 | m/z = 715.27 (C₅₁H₃₃N₅ = 715.84) |
| 3-79-1 | m/z = 714.28 (C₅₂H₃₄N₄ = 714.85) | 3-80-1 | m/z = 714.28 (C₅₂H₃₄N₄ = 714.85) |
| 3-81-1 | m/z = 715.27 (C₅₁H₃₃N₅ = 715.84) | 3-82-1 | m/z = 716.27 (C₅₀H₃₂N₆ = 716.83) |
| 3-83-1 | m/z = 716.27 (C₅₀H₃₂N₆ = 716.83) | 3-84-1 | m/z = 715.27 (C₅₁H₃₃N₅ = 715.84) |
| 3-85-1 | m/z = 715.27 (C₅₁H₃₃N₅ = 715.84) | 3-86-1 | m/z = 714.28 (C₅₂H₃₄N₄ = 714.85) |
| 3-87-1 | m/z = 714.28 (C₅₂H₃₄N₄ = 714.85) | 3-88-1 | m/z = 715.27 (C₅₁H₃₃N₅ = 715.84) |
| 3-89-1 | m/z = 716.27 (C₅₀H₃₂N₆ = 716.83) | 3-90-1 | m/z = 613.23 (C₄₃H₂₇N₅ = 613.71) |
| 3-91-1 | m/z = 640.24 (C₄₄H₂₈N₆ = 640.73) | 3-92-1 | m/z = 639.24 (C₄₅H₂₉N₅ = 639.75) |
| 3-93-1 | m/z = 639.24 (C₄₅H₂₉N₅ = 639.75) | 3-94-1 | m/z = 639.24 (C₄₅H₂₉N₅ = 639.75) |
| 3-95-1 | m/z = 638.25 (C₄₆H₃₀N₄ = 638.76) | 3-96-1 | m/z = 638.25 (C₄₆H₃₀N₄ = 638.76) |
| 3-97-1 | m/z = 639.24 (C₄₅H₂₉N₅ = 639.75) | 3-98-1 | m/z = 640.24 (C₄₄H₂₈N₆ = 640.73) |
| 3-99-1 | m/z = 716.27 (C₅₀H₃₂N₆ = 716.83) | 3-100-1 | m/z = 715.27 (C₅₁H₃₃N₅ = 715.84) |
| 3-101-1 | m/z = 715.27 (C₅₁H₃₃N₅ = 715.84) | 3-102-1 | m/z = 714.28 (C₅₂H₃₄N₄ = 714.85) |
| 3-103-1 | m/z = 714.28 (C₅₂H₃₄N₄ = 714.85) | 3-104-1 | m/z = 715.27 (C₅₁H₃₃N₅ = 715.84) |
| 3-105-1 | m/z = 716.27 (C₅₀H₃₂N₆ = 716.83) | 3-106-1 | m/z = 716.27 (C₅₀H₃₂N₆ = 716.83) |
| 3-107-1 | m/z = 715.27 (C₅₁H₃₃N₅ = 715.84) | 3-108-1 | m/z = 715.27 (C₅₁H₃₃N₅ = 715.84) |
| 3-109-1 | m/z = 714.28 (C₅₂H₃₄N₄ = 714.85) | 3-110-1 | m/z = 714.28 (C₅₂H₃₄N₄ = 714.85) |
| 3-111-1 | m/z = 715.27 (C₅₁H₃₃N₅ = 715.84) | 3-112-1 | m/z = 716.27 (C₅₀H₃₂N₆ = 716.83) |
| 3-113-1 | m/z = 613.23 (C₄₃H₂₇N₅ = 613.71) | 3-114-1 | m/z = 485.19 (C₃₅H₂₃N₃ = 485.58) |
| 3-115-1 | m/z = 535.20 (C₃₉H₂₅N₃ = 535.64) | 3-116-1 | m/z = 561.22 (C₄₁H₂₇N₃ = 561.67) |
| 3-117-1 | m/z = 640.24 (C₄₄H₂₈N₆ = 640.73) | 3-118-1 | m/z = 485.19 (C₃₅H₂₃N₃ = 485.58) |
| 3-119-1 | m/z = 535.20 (C₃₉H₂₅N₃ = 535.64) | 3-120-1 | m/z = 561.22 (C₄₁H₂₇N₃ = 561.67) |
| 3-121-1 | m/z = 640.24 (C₄₄H₂₈N₆ = 640.73) | 3-122-1 | m/z = 485.19 (C₃₅H₂₃N₃ = 485.58) |
| 3-123-1 | m/z = 535.20 (C₃₉H₂₅N₃ = 535.64) | 3-124-1 | m/z = 561.22 (C₄₁H₂₇N₃ = 561.67) |
| 3-125-1 | m/z = 640.24 (C₄₄H₂₈N₆ = 640.73) | 3-126-1 | m/z = 640.24 (C₄₄H₂₈N₆ = 640.73) |
| 3-127-1 | m/z = 535.20 (C₃₉H₂₅N₃ = 535.64) | 3-128-1 | m/z = 535.20 (C₃₉H₂₅N₃ = 535.64) |
| 4-1-1 | m/z = 485.19 (C₃₅H₂₃N₃ = 485.58) | 4-2-1 | m/z = 535.20 (C₃₉H₂₅N₃ = 535.64) |
| 4-3-1 | m/z = 561.22 (C₄₁H₂₇N₃ = 561.67) | 4-4-1 | m/z = 640.24 (C₄₄H₂₈N₆ = 640.73) |
| 4-5-1 | m/z = 485.19 (C₃₅H₂₃N₃ = 485.58) | 4-6-1 | m/z = 535.20 (C₃₉H₂₅N₃ = 535.64) |
| 4-7-1 | m/z = 561.22 (C₄₁H₂₇N₃ = 561.67) | 4-8-1 | m/z = 640.24 (C₄₄H₂₈N₆ = 640.73) |
| 4-9-1 | m/z = 485.19 (C₃₅H₂₃N₃ = 485.58) | 4-10-1 | m/z = 535.20 (C₃₉H₂₅N₃ = 535.64) |
| 4-11-1 | m/z = 561.22 (C₄₁H₂₇N₃ = 561.67) | 4-12-1 | m/z = 640.24 (C₄₄H₂₈N₆ = 640.73) |
| 4-13-1 | m/z = 485.19 (C₃₅H₂₃N₃ = 485.58) | 4-14-1 | m/z = 535.20 (C₃₉H₂₅N₃ = 535.64) |
| 4-15-1 | m/z = 561.22 (C₄₁H₂₇N₃ = 561.67) | 4-16-1 | m/z = 640.24 (C₄₄H₂₈N₆ = 640.73) |
| 4-17-1 | m/z = 485.19 (C₃₅H₂₃N₃ = 485.58) | 4-18-1 | m/z = 535.20 (C₃₉H₂₅N₃ = 535.64) |
| 4-19-1 | m/z = 561.22 (C₄₁H₂₇N₃ = 561.67) | 4-20-1 | m/z = 640.24 (C₄₄H₂₈N₆ = 640.73) |
| 4-21-1 | m/z = 485.19 (C₃₅H₂₃N₃ = 485.58) | 4-22-1 | m/z = 535.20 (C₃₉H₂₅N₃ = 535.64) |
| 4-23-1 | m/z = 561.22 (C₄₁H₂₇N₃ = 561.67) | 4-24-1 | m/z = 640.24 (C₄₄H₂₈N₆ = 640.73) |
| 4-25-1 | m/z = 485.19 (C₃₅H₂₃N₃ = 485.58) | 4-26-1 | m/z = 535.20 (C₃₉H₂₅N₃ = 535.64) |
| 4-27-1 | m/z = 561.22 (C₄₁H₂₇N₃ = 561.67) | 4-28-1 | m/z = 640.24 (C₄₄H₂₈N₆ = 640.73) |
| 5-1-1 | m/z = 653.26 (C₄₆H₃₁N₅ = 653.77) | 5-3-1 | m/z = 652.26 (C₄₇H₃₂N₄ = 652.78) |
| 5-2-1 | m/z = 728.29 (C₅₃H₃₆N₄ = 728.88) | 5-4-1 | m/z = 728.29 (C₅₃H₃₆N₄ = 728.88) |

### Manufacture and Evaluation of Organic Electronic Element

### I. Manufacture and Test of Green Organic Light Emitting Element (Phosphorescent Host)

### Example 1-1 Green Organic Light Emitting Element (Phosphorescent Host)

An organic electronic light emitting element was manufactured by an ordinary method using the compound obtained through synthesis as a host material for a light emitting layer. First, a film of N¹-(naphthalen-2-yl)-N⁴,N⁴-bis(4-(naphthalen-2-yl(phenyl)amino)phenyl)-N¹-phenylbenzene-1,4-diamine (hereinafter, abbreviated as “2-TNATA”) as a hole injection layer was vacuum-deposited with a thickness of 60 nm on an ITO layer (anode) formed on a galas substrate. Then, 4,4-bis[N-(1-naphthyl)-N-phenylamino]biphenyl (hereinafter, abbreviated as “-NPD”) as a hole transport compound was vacuum-deposited on the hole injection layer to form a hole transport layer with a thickness of 60 nm. Subsequently, a light emitting layer with a thickness of 30 nm was formed on the hole transport layer by doping an upper portion of the hole transport layer with the compound 1-1-1 of the present invention as a host and Ir(ppy)₃ [tris(2-phenylpyridine)-iridium] as a dopant at a weight ratio of 95:5. Then, (1.1′-bisphenyl)-4-olato)bis(2-methyl-8-quinolinolato)aluminum (hereinafter, abbreviated as “BAlq”) was vacuum-deposited with a thickness of 10 nm for a hole blocking layer, and tris(8-quinolinol)aluminum (hereinafter, abbreviated as “Alq3”) was formed with a thickness of 40 nm for an electron injection layer. Thereafter, LiF as halogenated alkali metal was deposited with a thickness of 0.2 nm, and subsequently Al was deposited with a thickness of 150 nm, thereby using this Al/LiF as a cathode. In this way, an organic electronic light emitting element was manufactured.

### Example 1-2 to Example 1-312 Green Organic Light Emitting Element (Phosphorescent Host)

An organic electronic light emitting element was manufactured by the same method as in Example 1-1 except that, instead of compound 1-1-1 of the present invention, one of compounds 1-2-1 to 1-28-1, 2-1-1 to 2-128-1, 3-1-1 to 3-128-1, and 4-1-1 to 4-28-1 of the present invention listed on table 5 below was used as a phosphorescent host material for a light emitting layer.

### Comparative Example 1-1

An organic electronic light emitting element was manufactured by the same method as in Example 1-1 except that, instead of compound 1-1-1 of the present invention, comparative compound A [4,4′-N,N′-dicarbazole-biphenyl (CBP)] below was used as a phosphorescent host material for a light emitting layer.

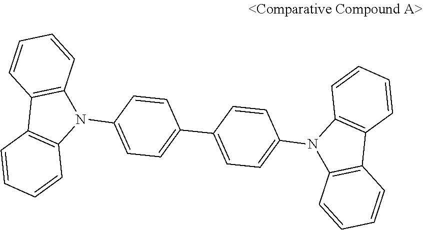 [SMILES: c1ccc2c(c1)c1ccccc1n2-c1ccc(-c2ccc(-n3c4ccccc4c4ccccc43)cc2)cc1]

### Comparative Example 1-2

An organic electronic light emitting element was manufactured by the same method as in Example 1-1 except that, instead of compound 1-1-1 of the present invention, comparative compound B below was used as a phosphorescent host material for a light emitting layer.

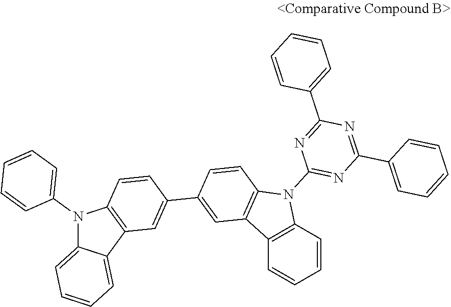 [SMILES: c1ccc(-c2nc(-c3ccccc3)nc(-n3c4ccccc4c4cc(-c5ccc6c(c5)c5ccccc5n6-c5ccccc5)ccc43)n2)cc1]

### Comparative Example 1-3

An organic electronic light emitting element was manufactured by the same method as in Example 1-1 except that, instead of compound 1-1-1 of the present invention, comparative compound C below was used as a phosphorescent host material for a light emitting layer.

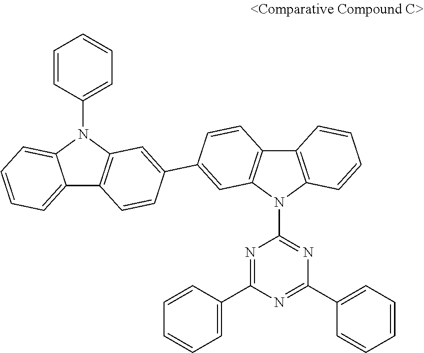 [SMILES: c1ccc(-c2nc(-c3ccccc3)nc(-n3c4ccccc4c4ccc(-c5ccc6c7ccccc7n(-c7ccccc7)c6c5)cc43)n2)cc1]

### Comparative Example 1-4

An organic electronic light emitting element was manufactured by the same method as in Example 1-1 except that, instead of compound 1-1-1 of the present invention, comparative compound D below was used as a phosphorescent host material for a light emitting layer.

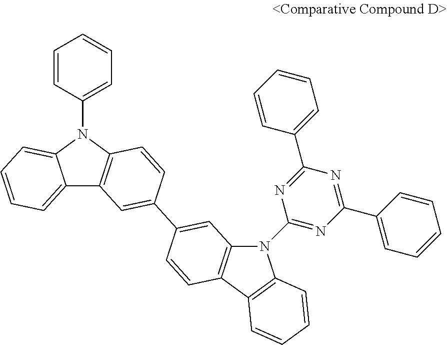 [SMILES: c1ccc(-c2nc(-c3ccccc3)nc(-n3c4ccccc4c4ccc(-c5ccc6c(c5)c5ccccc5n6-c5ccccc5)cc43)n2)cc1]

A forward bias DC voltage was applied to the organic electronic light emitting elements manufactured in Examples 1-1 to 1-312 and Comparative Examples 1-1 to 1-4 to measure electro-luminescence (EL) characteristics thereof by PR-650 (Photoresearch), and the T95 lifetime was measured by lifetime measuring equipments (Mcscience) at reference brightness of 5000 cd/m². Table 1-5 below shows the manufacture of elements and evaluation results thereof.

TABLE 1-5

| | | | Current | Brightness | | Lifetime | CIE |
|---|---|---|---|---|---|---|---|
| | Compound | Voltage | Density | (cd/m2) | Efficiency | T(95) | (x, y) |
| Comparative | Compound | 5.7 | 21.7 | 5000.0 | 23.0 | 68.3 | (0.31, |
| Example (1-1) | (A) | | | | | | 0.60) |
| Comparative | Compound | 5.3 | 17.6 | 5000.0 | 28.5 | 89.0 | (0.31, |
| Example (1-2) | (B) | | | | | | 0.61) |
| Comparative | Compound | 5.5 | 18.3 | 5000.0 | 27.3 | 80.7 | (0.31, |
| Example (1-3) | (C) | | | | | | 0.60) |
| Comparative | Compound | 5.6 | 18.3 | 5000.0 | 27.4 | 87.9 | (0.33, |
| Example (1-4) | (D) | | | | | | 0.61) |
| Example (1-1) | Compound | 4.7 | 15.2 | 5000.0 | 33.0 | 126.1 | (0.30, |
| | (1-1-1) | | | | | | 0.60) |
| Example (1-2) | Compound | 4.8 | 14.8 | 5000.0 | 33.8 | 97.7 | (0.31, |
| | (1-2-1) | | | | | | 0.61) |
| Example (1-3) | Compound | 4.7 | 15.7 | 5000.0 | 31.9 | 130.3 | (0.31, |
| | (1-3-1) | | | | | | 0.60) |
| Example (1-4) | Compound | 4.9 | 16.6 | 5000.0 | 30.1 | 117.4 | (0.33, |
| | (1-4-1) | | | | | | 0.61) |
| Example (1-5) | Compound | 4.7 | 14.3 | 5000.0 | 34.9 | 144.9 | (0.32, |
| | (1-5-1) | | | | | | 0.61) |
| Example (1-6) | Compound | 5.0 | 14.7 | 5000.0 | 33.9 | 119.7 | (0.33, |
| | (1-6-1) | | | | | | 0.60) |
| Example (1-7) | Compound | 4.9 | 16.2 | 5000.0 | 30.9 | 124.5 | (0.32, |
| | (1-7-1) | | | | | | 0.61) |
| Example (1-8) | Compound | 4.7 | 16.0 | 5000.0 | 31.3 | 120.7 | (0.31, |
| | (1-8-1) | | | | | | 0.60) |
| Example (1-9) | Compound | 4.9 | 16.5 | 5000.0 | 30.3 | 105.4 | (0.31, |
| | (1-9-1) | | | | | | 0.61) |
| Example (1-10) | Compound | 4.9 | 14.3 | 5000.0 | 34.8 | 103.9 | (0.31, |
| | (1-10-1) | | | | | | 0.60) |
| Example (1-11) | Compound | 4.8 | 14.7 | 5000.0 | 34.1 | 135.5 | (0.33, |
| | (1-11-1) | | | | | | 0.61) |
| Example (1-12) | Compound | 4.8 | 15.7 | 5000.0 | 31.9 | 130.1 | (0.30, |
| | (1-12-1) | | | | | | 0.60) |
| Example (1-13) | Compound | 4.8 | 16.0 | 5000.0 | 31.2 | 146.7 | (0.31, |
| | (1-13-1) | | | | | | 0.61) |
| Example (1-14) | Compound | 4.9 | 16.2 | 5000.0 | 30.8 | 148.4 | (0.31, |
| | (1-14-1) | | | | | | 0.60) |
| Example (1-15) | Compound | 4.8 | 15.6 | 5000.0 | 32.1 | 128.8 | (0.33, |
| | (1-15-1) | | | | | | 0.61) |
| Example (1-16) | Compound | 4.9 | 15.9 | 5000.0 | 31.4 | 132.7 | (0.32, |
| | (1-16-1) | | | | | | 0.61) |
| Example (1-17) | Compound | 5.0 | 15.8 | 5000.0 | 31.6 | 128.6 | (0.33, |
| | (1-17-1) | | | | | | 0.60) |
| Example (1-18) | Compound | 5.0 | 16.5 | 5000.0 | 30.3 | 134.9 | (0.32, |
| | (1-18-1) | | | | | | 0.61) |
| Example (1-19) | Compound | 5.0 | 16.0 | 5000.0 | 31.3 | 141.4 | (0.31, |
| | (1-19-1) | | | | | | 0.60) |
| Example (1-20) | Compound | 4.8 | 16.1 | 5000.0 | 31.0 | 128.6 | (0.31, |
| | (1-20-1) | | | | | | 0.61) |
| Example (1-21) | Compound | 4.7 | 15.5 | 5000.0 | 32.3 | 107.5 | (0.31, |
| | (1-21-1) | | | | | | 0.60) |
| Example (1-22) | Compound | 4.8 | 14.4 | 5000.0 | 34.6 | 103.7 | (0.33, |
| | (1-22-1) | | | | | | 0.61) |
| Example (1-23) | Compound | 4.8 | 14.3 | 5000.0 | 35.0 | 106.1 | (0.30, |
| | (1-23-1) | | | | | | 0.60) |
| Example (1-24) | Compound | 4.8 | 14.3 | 5000.0 | 35.0 | 103.2 | (0.31, |
| | (1-24-1) | | | | | | 0.61) |
| Example (1-25) | Compound | 4.8 | 14.8 | 5000.0 | 33.8 | 136.3 | (0.31, |
| | (1-25-1) | | | | | | 0.60) |
| Example (1-26) | Compound | 4.9 | 14.7 | 5000.0 | 33.9 | 128.1 | (0.33, |
| | (1-26-1) | | | | | | 0.61) |
| Example (1-27) | Compound | 4.8 | 15.7 | 5000.0 | 31.8 | 99.5 | (0.32, |
| | (1-27-1) | | | | | | 0.61) |
| Example (1-28) | Compound | 4.8 | 14.9 | 5000.0 | 33.5 | 149.9 | (0.33, |
| | (1-28-1) | | | | | | 0.60) |
| Example (1-29) | Compound | 4.8 | 13.0 | 5000.0 | 38.5 | 135.4 | (0.31) |
| | (2-1-1) | | | | | | 0.61) |
| Example (1-30) | Compound | 4.8 | 13.1 | 5000.0 | 38.1 | 142.5 | (0.31, |
| | (2-2-1) | | | | | | 0.60) |
| Example (1-31) | Compound | 4.9 | 14.2 | 5000.0 | 35.3 | 141.9 | (0.33, |
| | (2-3-1) | | | | | | 0.61) |
| Example (1-32) | Compound | 4.9 | 14.2 | 5000.0 | 35.1 | 107.6 | (0.32) |
| | (2-4-1) | | | | | | 0.61) |
| Example (1-33) | Compound | 4.9 | 12.7 | 5000.0 | 39.5 | 92.0 | (0.33, |
| | (2-5-1) | | | | | | 0.60) |
| Example (1-34) | Compound | 4.7 | 13.0 | 5000.0 | 38.5 | 145.0 | (0.32, |
| | (2-6-1) | | | | | | 0.61) |
| Example (1-35) | Compound | 4.8 | 14.2 | 5000.0 | 35.1 | 139.0 | (0.31, |
| | (2-7-1) | | | | | | 0.60) |
| Example (1-36) | Compound | 5.0 | 14.0 | 5000.0 | 35.6 | 140.4 | (0.31, |
| | (2-8-1) | | | | | | 0.61) |
| Example (1-37) | Compound | 5.0 | 14.2 | 5000.0 | 35.1 | 91.1 | (0.31, |
| | (2-9-1) | | | | | | 0.60) |
| Example (1-38) | Compound | 4.9 | 12.6 | 5000.0 | 39.8 | 121.7 | (0.33, |
| | (2-10-1) | | | | | | 0.61) |
| Example (1-39) | Compound | 4.8 | 13.1 | 5000.0 | 38.2 | 147.9 | (0.30, |
| | (2-11-1) | | | | | | 0.60) |
| Example (1-40) | Compound | 4.9 | 12.7 | 5000.0 | 39.3 | 111.0 | (0.31, |
| | (2-12-1) | | | | | | 0.61) |
| Example (1-41) | Compound | 4.9 | 13.2 | 5000.0 | 38.0 | 107.6 | (0.31, |
| | (2-13-1) | | | | | | 0.60) |
| Example (1-42) | Compound | 4.9 | 14.1 | 5000.0 | 35.5 | 126.2 | (0.33, |
| | (2-14-1) | | | | | | 0.61) |
| Example (1-43) | Compound | 4.7 | 12.6 | 5000.0 | 39.8 | 107.0 | (0.32, |
| | (2-15-1) | | | | | | 0.61) |
| Example (1-44) | Compound | 5.0 | 13.0 | 5000.0 | 38.4 | 108.8 | (0.33, |
| | (2-16-1) | | | | | | 0.60) |
| Example (1-45) | Compound | 4.9 | 13.2 | 5000.0 | 37.8 | 96.7 | (0.32, |
| | (2-17-1) | | | | | | 0.61) |
| Example (1-46) | Compound | 4.7 | 13.5 | 5000.0 | 37.2 | 131.8 | (0.31, |
| | (2-18-1) | | | | | | 0.60) |
| Example (1-47) | Compound | 4.9 | 13.5 | 5000.0 | 36.9 | 138.9 | (0.31, |
| | (2-19-1) | | | | | | 0.61) |
| Example (1-48) | Compound | 4.9 | 13.1 | 5000.0 | 38.2 | 97.3 | (0.31, |
| | (2-20-1) | | | | | | 0.60) |
| Example (1-49) | Compound | 4.7 | 14.0 | 5000.0 | 35.6 | 111.3 | (0.33, |
| | (2-21-1) | | | | | | 0.61) |
| Example (1-50) | Compound | 4.9 | 14.2 | 5000.0 | 35.3 | 94.5 | (0.30, |
| | (2-22-1) | | | | | | 0.60) |
| Example (1-51) | Compound | 4.9 | 12.8 | 5000.0 | 39.0 | 142.4 | (0.31, |
| | (2-23-1) | | | | | | 0.61) |
| Example (1-52) | Compound | 4.8 | 14.1 | 5000.0 | 35.4 | 118.5 | (0.31, |
| | (2-24-1) | | | | | | 0.60) |
| Example (1-53) | Compound | 4.8 | 13.5 | 5000.0 | 37.1 | 145.9 | (0.33, |
| | (2-25-1) | | | | | | 0.61) |
| Example (1-54) | Compound | 4.8 | 12.8 | 5000.0 | 39.0 | 135.6 | (0.32, |
| | (2-26-1) | | | | | | 0.61) |
| Example (1-55) | Compound | 4.9 | 13.9 | 5000.0 | 35.9 | 95.5 | (0.33, |
| | (2-27-1) | | | | | | 0.60) |
| Example(1-56) | Compound | 4.9 | 13.3 | 5000.0 | 37.5 | 102.5 | (0.32, |
| | (2-28-1) | | | | | | 0.61) |
| Example(1-57) | Compound | 5.0 | 12.9 | 5000.0 | 38.6 | 102.6 | (0.31, |
| | (2-29-1) | | | | | | 0.60) |
| Example (1-58) | Compound | 5.0 | 14.3 | 5000.0 | 35.0 | 92.9 | (0.33, |
| | (2-30-1) | | | | | | 0.61) |
| Example (1-59) | Compound | 4.9 | 13.9 | 5000.0 | 35.9 | 95.9 | (0.30, |
| | (2-31-1) | | | | | | 0.60) |
| Example (1-60) | Compound | 5.0 | 13.5 | 5000.0 | 36.9 | 118.6 | (0.31, |
| | (2-32-1) | | | | | | 0.61) |
| Example (1-61) | Compound | 4.7 | 13.8 | 5000.0 | 36.1 | 117.9 | (0.31, |
| | (2-33-1) | | | | | | 0.60) |
| Example (1-62) | Compound | 4.7 | 12.8 | 5000.0 | 39.1 | 93.8 | (0.33, |
| | (2-34-1) | | | | | | 0.61) |
| Example (1-63) | Compound | 4.7 | 13.3 | 5000.0 | 37.6 | 147.3 | (0.32, |
| | (2-35-1) | | | | | | 0.61) |
| Example (1-64) | Compound | 4.9 | 14.2 | 5000.0 | 35.2 | 95.7 | (0.33, |
| | (2-36-1) | | | | | | 0.60) |
| Example (1-65) | Compound | 4.9 | 13.3 | 5000.0 | 37.5 | 127.5 | (0.32, |
| | (2-37-1) | | | | | | 0.61) |
| Example(1-66) | Compound | 4.7 | 12.9 | 5000.0 | 38.7 | 129.9 | (0.31, |
| | (2-38-1) | | | | | | 0.60) |
| Example (1-67) | Compound | 4.9 | 13.0 | 5000.0 | 38.6 | 99.0 | (0.31, |
| | (2-39-1) | | | | | | 0.61) |
| Example (1-68) | Compound | 4.9 | 13.7 | 5000.0 | 36.4 | 117.9 | (0.31, |
| | (2-40-1) | | | | | | 0.60) |
| Example (1-69) | Compound | 4.7 | 14.0 | 5000.0 | 35.7 | 145.7 | (0.33, |
| | (2-41-1) | | | | | | 0.61) |
| Example (1-70) | Compound | 5.0 | 12.7 | 5000.0 | 39.5 | 118.2 | (0.30, |
| | (2-42-1) | | | | | | 0.60) |
| Example (1-71) | Compound | 4.8 | 13.3 | 5000.0 | 37.6 | 124.2 | (0.31, |
| | (2-43-1) | | | | | | 0.61) |
| Example (1-72) | Compound | 4.8 | 12.8 | 5000.0 | 38.9 | 118.0 | (0.31, |
| | (2-44-1) | | | | | | 0.60) |
| Example(1-73) | Compound | 4.9 | 12.9 | 5000.0 | 38.9 | 113.7 | (0.33, |
| | (2-45-1) | | | | | | 0.61) |
| Example (1-74) | Compound | 4.9 | 12.8 | 5000.0 | 39.2 | 121.7 | (0.32, |
| | (2-46-1) | | | | | | 0.61) |
| Example (1-75) | Compound | 4.9 | 13.2 | 5000.0 | 37.9 | 101.2 | (0.33, |
| | (2-47-1) | | | | | | 0.61) |
| Example (1-76) | Compound | 4.8 | 13.3 | 5000.0 | 37.5 | 114.4 | (0.30, |
| | (2-48-1) | | | | | | 0.60) |
| Example (1-77) | Compound | 4.9 | 12.8 | 5000.0 | 39.2 | 125.4 | (0.31, |
| | (2-49-1) | | | | | | 0.61) |
| Example (1-78) | Compound | 5.0 | 12.8 | 5000.0 | 39.1 | 106.0 | (0.31, |
| | (2-50-1) | | | | | | 0.60) |
| Example (1-79) | Compound | 4.9 | 13.1 | 5000.0 | 38.1 | 120.9 | (0.31, |
| | (2-51-1) | | | | | | 0.61) |
| Example (1-80) | Compound | 4.9 | 13.5 | 5000.0 | 37.0 | 115.0 | (0.31, |
| | (2-52-1) | | | | | | 0.60) |
| Example (1-81) | Compound | 5.0 | 13.9 | 5000.0 | 35.9 | 101.6 | (0.33, |
| | (2-53-1) | | | | | | 0.61) |
| Example(1-82) | Compound | 4.9 | 13.1 | 5000.0 | 38.1 | 137.3 | (0.32, |
| | (2-54-1) | | | | | | 0.61) |
| Example (1-83) | Compound | 4.9 | 13.4 | 5000.0 | 37.5 | 130.3 | (0.33, |
| | (2-55-1) | | | | | | 0.60) |
| Example (1-84) | Compound | 5.0 | 13.0 | 5000.0 | 38.6 | 94.9 | (0.32, |
| | (2-56-1) | | | | | | 0.61) |
| Example (1-85) | Compound | 4.7 | 14.1 | 5000.0 | 35.5 | 98.4 | (0.31, |
| | (2-57-1) | | | | | | 0.60) |
| Example (1-86) | Compound | 4.9 | 12.9 | 5000.0 | 38.8 | 122.9 | (0.31, |
| | (2-58-1) | | | | | | 0.61) |
| Example (1-87) | Compound | 4.9 | 13.6 | 5000.0 | 36.8 | 96.1 | (0.31, |
| | (2-59-1) | | | | | | 0.60) |
| Example (1-88) | Compound | 4.8 | 13.7 | 5000.0 | 36.5 | 125.9 | (0.33, |
| | (2-60-1) | | | | | | 0.61) |
| Example (1-89) | Compound | 4.7 | 13.2 | 5000.0 | 38.0 | 126.9 | (0.30, |
| | (2-61-1) | | | | | | 0.60) |
| Example (1-90) | Compound | 4.8 | 13.4 | 5000.0 | 37.3 | 134.4 | (0.31, |
| | (2-62-1) | | | | | | 0.61) |
| Example (1-91) | Compound | 4.9 | 14.2 | 5000.0 | 35.2 | 102.1 | (0.31, |
| | (2-63-1) | | | | | | 0.60) |
| Example (1-92) | Compound | 4.7 | 12.7 | 5000.0 | 39.3 | 125.0 | (0.33, |
| | (2-64-1) | | | | | | 0.61) |
| Example (1-93) | Compound | 5.0 | 13.1 | 5000.0 | 38.1 | 105.4 | (0.32, |
| | (2-65-1) | | | | | | 0.61) |
| Example (1-94) | Compound | 4.8 | 13.7 | 5000.0 | 36.4 | 133.3 | (0.33, |
| | (2-66-1) | | | | | | 0.60) |
| Example (1-95) | Compound | 5.0 | 12.6 | 5000.0 | 39.8 | 115.3 | (0.32, |
| | (2-67-1) | | | | | | 0.61) |
| Example (1-96) | Compound | 5.0 | 14.1 | 5000.0 | 35.5 | 134.1 | (0.31, |
| | (2-68-1) | | | | | | 0.60) |
| Example (1-97) | Compound | 5.0 | 12.8 | 5000.0 | 38.9 | 108.9 | (0.31, |
| | (2-69-1) | | | | | | 0.61) |
| Example (1-98) | Compound | 5.0 | 14.1 | 5000.0 | 35.4 | 132.5 | (0.31, |
| | (2-70-1) | | | | | | 0.60) |
| Example (1-99) | Compound | 5.0 | 12.6 | 5000.0 | 39.6 | 145.3 | (0.33, |
| | (2-71-1) | | | | | | 0.61) |
| Example | Compound | 5.0 | 12.9 | 5000.0 | 38.7 | 122.5 | (0.30, |
| (1-100) | (2-72-1) | | | | | | 0.60) |
| Example | Compound | 4.9 | 14.0 | 5000.0 | 35.8 | 106.7 | (0.31, |
| (1-101) | (2-73-1) | | | | | | 0.61) |
| Example | Compound | 4.8 | 14.2 | 5000.0 | 35.3 | 131.9 | (0.31, |
| (1-102) | (2-74-1) | | | | | | 0.60) |
| Example | Compound | 5.0 | 14.1 | 5000.0 | 35.4 | 96.0 | (0.33, |
| (1-103) | (2-75-1) | | | | | | 0.61) |
| Example | Compound | 5.0 | 13.8 | 5000.0 | 36.3 | 106.4 | (0.32, |
| (1-104) | (2-76-1) | | | | | | 0.61) |
| Example | Compound | 4.9 | 13.3 | 5000.0 | 37.7 | 126.6 | (0.33, |
| (1-105) | (2-77-1) | | | | | | 0.60) |
| Example | Compound | 4.9 | 14.2 | 5000.0 | 35.3 | 121.1 | (0.32, |
| (1-106) | (2-78-1) | | | | | | 0.61) |
| Example | Compound | 4.9 | 14.1 | 5000.0 | 35.3 | 130.9 | (0.31, |
| (1-107) | (2-79-1) | | | | | | 0.60) |
| Example | Compound | 4.7 | 13.0 | 5000.0 | 38.4 | 118.6 | (0.33, |
| (1-108) | (2-80-1) | | | | | | 0.61) |
| Example | Compound | 5.0 | 14.0 | 5000.0 | 35.8 | 127.4 | (0.30, |
| (1-109) | (2-81-1) | | | | | | 0.60) |
| Example | Compound | 4.9 | 13.0 | 5000.0 | 38.3 | 119.9 | (0.31, |
| (1-110) | (2-82-1) | | | | | | 0.61) |
| Example | Compound | 4.9 | 13.9 | 5000.0 | 36.0 | 116.8 | (0.31, |
| (1-111) | (2-83-1) | | | | | | 0.60) |
| Example | Compound | 4.9 | 13.5 | 5000.0 | 36.9 | 110.5 | (0.33, |
| (1-112) | (2-84-1) | | | | | | 0.61) |
| Example | Compound | 4.8 | 13.1 | 5000.0 | 38.3 | 92.8 | (0.32, |
| (1-113) | (2-85-1) | | | | | | 0.61) |
| Example | Compound | 4.9 | 13.2 | 5000.0 | 38.0 | 97.6 | (0.33, |
| (1-114) | (2-86-1) | | | | | | 0.60) |
| Example | Compound | 4.8 | 13.9 | 5000.0 | 35.9 | 132.0 | (0.32, |
| (1-115) | (2-87-1) | | | | | | 0.61) |
| Example | Compound | 4.7 | 12.7 | 5000.0 | 39.4 | 126.3 | (0.31, |
| (1-116) | (2-88-1) | | | | | | 0.60) |
| Example | Compound | 4.9 | 13.0 | 5000.0 | 38.5 | 131.8 | (0.31, |
| (1-117) | (2-89-1) | | | | | | 0.61) |
| Example | Compound | 4.8 | 13.9 | 5000.0 | 35.9 | 139.0 | (0.31, |
| (1-118) | (2-90-1) | | | | | | 0.60) |
| Example | Compound | 4.7 | 12.6 | 5000.0 | 39.8 | 98.8 | (0.33, |
| (1-119) | (2-91-1) | | | | | | 0.61) |
| Example | Compound | 4.8 | 13.7 | 5000.0 | 36.4 | 140.1 | (0.30, |
| (1-120) | (2-92-1) | | | | | | 0.60) |
| Example | Compound | 4.8 | 12.8 | 5000.0 | 39.1 | 97.2 | (0.31, |
| (1-121) | (2-93-1) | | | | | | 0.61) |
| Example | Compound | 4.7 | 12.7 | 5000.0 | 39.4 | 138.7 | (0.31, |
| (1-122) | (2-94-1) | | | | | | 0.60) |
| Example | Compound | 5.0 | 12.5 | 5000.0 | 39.9 | 98.2 | (0.33, |
| (1-123) | (2-95-1) | | | | | | 0.61) |
| Example | Compound | 4.9 | 13.8 | 5000.0 | 36.2 | 146.5 | (0.32, |
| (1-124) | (2-96-1) | | | | | | 0.61) |
| Example | Compound | 4.8 | 12.8 | 5000.0 | 39.0 | 112.8 | (0.33, |
| (1-125) | (2-97-1) | | | | | | 0.61) |
| Example | Compound | 4.8 | 14.0 | 5000.0 | 35.6 | 148.1 | (0.30, |
| (1-126) | (2-98-1) | | | | | | 0.60) |
| Example | Compound | 4.8 | 13.5 | 5000.0 | 37.0 | 130.1 | (0.32, |
| (1-127) | (2-99-1) | | | | | | 0.61) |
| Example | Compound | 4.8 | 12.6 | 5000.0 | 39.7 | 94.9 | (0.31, |
| (1-128) | (2-100-1) | | | | | | 0.60) |
| Example | Compound | 4.7 | 12.9 | 5000.0 | 38.7 | 93.9 | (0.30, |
| (1-129) | (2-101-1) | | | | | | 0.60) |
| Example | Compound | 5.0 | 12.7 | 5000.0 | 39.4 | 119.9 | (0.31, |
| (1-130) | (2-102-1) | | | | | | 0.61) |
| Example | Compound | 5.0 | 13.6 | 5000.0 | 36.8 | 122.9 | (0.31, |
| (1-131) | (2-103-1) | | | | | | 0.60) |
| Example | Compound | 4.8 | 12.8 | 5000.0 | 39.0 | 92.9 | (0.33, |
| (1-132) | (2-104-1) | | | | | | 0.61) |
| Example | Compound | 4.8 | 13.4 | 5000.0 | 37.2 | 113.9 | (0.32, |
| (1-133) | (2-105-1) | | | | | | 0.61) |
| Example | Compound | 4.8 | 14.0 | 5000.0 | 35.7 | 129.5 | (0.33, |
| (1-134) | (2-106-1) | | | | | | 0.60) |
| Example | Compound | 4.9 | 13.6 | 5000.0 | 36.7 | 122.5 | (0.32, |
| (1-135) | (2-107-1) | | | | | | 0.61) |
| Example | Compound | 5.0 | 13.1 | 5000.0 | 38.3 | 136.7 | (0.31, |
| (1-136) | (2-108-1) | | | | | | 0.60) |
| Example | Compound | 4.9 | 13.9 | 5000.0 | 35.9 | 130.5 | (0.31, |
| (1-137) | (2-109-1) | | | | | | 0.61) |
| Example | Compound | 4.7 | 13.3 | 5000.0 | 37.7 | 108.3 | (0.31, |
| (1-138) | (2-110-1) | | | | | | 0.60) |
| Example | Compound | 4.9 | 13.6 | 5000.0 | 36.9 | 91.5 | (0.33, |
| (1-139) | (2-111-1) | | | | | | 0.61) |
| Example | Compound | 4.8 | 12.6 | 5000.0 | 39.7 | 103.6 | (0.30, |
| (1-140) | (2-112-1) | | | | | | 0.60) |
| Example | Compound | 4.9 | 12.7 | 5000.0 | 39.4 | 93.5 | (0.31, |
| (1-141) | (2-113-1) | | | | | | 0.61) |
| Example | Compound | 4.9 | 14.3 | 5000.0 | 35.0 | 118.0 | (0.31, |
| (1-142) | (2-114-1) | | | | | | 0.60) |
| Example | Compound | 4.9 | 12.6 | 5000.0 | 39.6 | 101.5 | (0.33, |
| (1-143) | (2-115-1) | | | | | | 0.61) |
| Example | Compound | 4.7 | 12.7 | 5000.0 | 39.4 | 147.2 | (0.32, |
| (1-144) | (2-116-1) | | | | | | 0.61) |
| Example | Compound | 4.8 | 13.2 | 5000.0 | 37.8 | 140.0 | (0.33, |
| (1-145) | (2-117-1) | | | | | | 0.60) |
| Example | Compound | 4.7 | 14.0 | 5000.0 | 35.8 | 109.7 | (0.32, |
| (1-146) | (2-118-1) | | | | | | 0.61) |
| Example | Compound | 4.8 | 13.7 | 5000.0 | 36.5 | 91.3 | (0.31, |
| (1-147) | (2-119-1) | | | | | | 0.60) |
| Example | Compound | 4.8 | 12.9 | 5000.0 | 38.8 | 146.0 | (0.31, |
| (1-148) | (2-120-1) | | | | | | 0.61) |
| Example | Compound | 4.8 | 13.2 | 5000.0 | 37.8 | 111.2 | (0.31, |
| (1-149) | (2-121-1) | | | | | | 0.60) |
| Example | Compound | 4.8 | 13.6 | 5000.0 | 36.8 | 120.4 | (0.33, |
| (1-150) | (2-122-1) | | | | | | 0.61) |
| Example | Compound | 4.9 | 14.2 | 5000.0 | 35.1 | 123.2 | (0.30, |
| (1-151) | (2-123-1) | | | | | | 0.60) |
| Example | Compound | 4.7 | 12.6 | 5000.0 | 39.6 | 144.2 | (0.31, |
| (1-152) | (2-124-1) | | | | | | 0.61) |
| Example | Compound | 4.9 | 13.6 | 5000.0 | 36.6 | 93.5 | (0.31, |
| (1-153) | (2-125-1) | | | | | | 0.60) |
| Example | Compound | 4.8 | 13.9 | 5000.0 | 36.1 | 114.3 | (0.33, |
| (1-154) | (2-126-1) | | | | | | 0.61) |
| Example | Compound | 4.8 | 14.3 | 5000.0 | 35.1 | 126.6 | (0.32, |
| (1-155) | (2-127-1) | | | | | | 0.61) |
| Example | Compound | 4.9 | 12.6 | 5000.0 | 39.8 | 130.2 | (0.33, |
| (1-156) | (2-128-1) | | | | | | 0.60) |
| Example | Compound | 4.7 | 14.2 | 5000.0 | 35.3 | 137.5 | (0.31, |
| (1-157) | (3-1-1) | | | | | | 0.61) |
| Example | Compound | 4.8 | 14.1 | 5000.0 | 35.5 | 111.6 | (0.31, |
| (1-158) | (3-2-1) | | | | | | 0.60) |
| Example | Compound | 4.7 | 13.0 | 5000.0 | 38.4 | 91.9 | (0.33, |
| (1-159) | (3-3-1) | | | | | | 0.61) |
| Example | Compound | 4.7 | 13.6 | 5000.0 | 36.8 | 128.4 | (0.32, |
| (1-160) | (3-4-1) | | | | | | 0.61) |
| Example | Compound | 4.9 | 13.7 | 5000.0 | 36.4 | 116.9 | (0.33, |
| (1-161) | (3-5-1) | | | | | | 0.60) |
| Example | Compound | 5.0 | 13.9 | 5000.0 | 36.0 | 138.7 | (0.32, |
| (1-162) | (3-6-1) | | | | | | 0.61) |
| Example | Compound | 5.0 | 12.5 | 5000.0 | 40.0 | 107.2 | (0.31, |
| (1-163) | (3-7-1) | | | | | | 0.60) |
| Example | Compound | 4.8 | 12.7 | 5000.0 | 39.4 | 147.2 | (0.31, |
| (1-164) | (3-8-1) | | | | | | 0.61) |
| Example | Compound | 4.9 | 13.9 | 5000.0 | 36.0 | 149.6 | (0.31, |
| (1-165) | (3-9-1) | | | | | | 0.60) |
| Example | Compound | 4.7 | 12.5 | 5000.0 | 40.0 | 122.0 | (0.33, |
| (1-166) | (3-10-1) | | | | | | 0.61) |
| Example | Compound | 4.7 | 14.1 | 5000.0 | 35.6 | 138.2 | (0.30, |
| (1-167) | (3-11-1) | | | | | | 0.60) |
| Example | Compound | 4.8 | 13.5 | 5000.0 | 36.9 | 104.9 | (0.31, |
| (1-168) | (3-12-1) | | | | | | 0.61) |
| Example | Compound | 5.0 | 14.0 | 5000.0 | 35.7 | 107.7 | (0.31, |
| (1-169) | (3-13-1) | | | | | | 0.60) |
| Example | Compound | 4.7 | 13.0 | 5000.0 | 38.3 | 96.1 | (0.33, |
| (1-170) | (3-14-1) | | | | | | 0.61) |
| Example | Compound | 4.9 | 13.2 | 5000.0 | 37.9 | 133.2 | (0.32, |
| (1-171) | (3-15-1) | | | | | | 0.61) |
| Example | Compound | 4.7 | 12.9 | 5000.0 | 38.7 | 142.2 | (0.33, |
| (1-172) | (3-16-1) | | | | | | 0.60) |
| Example | Compound | 4.9 | 12.9 | 5000.0 | 38.8 | 100.1 | (0.32, |
| (1-173) | (3-17-1) | | | | | | 0.61) |
| Example | Compound | 4.8 | 13.4 | 5000.0 | 37.4 | 95.5 | (0.31, |
| (1-174) | (3-18-1) | | | | | | 0.60) |
| Example | Compound | 4.9 | 13.7 | 5000.0 | 36.4 | 107.2 | (0.31, |
| (1-175) | (3-19-1) | | | | | | 0.61) |
| Example | Compound | 4.9 | 12.7 | 5000.0 | 39.4 | 97.0 | (0.31, |
| (1-176) | (3-20-1) | | | | | | 0.60) |
| Example | Compound | 4.9 | 12.5 | 5000.0 | 39.9 | 103.9 | (0.33, |
| (1-177) | (3-21-1) | | | | | | 0.61) |
| Example | Compound | 4.9 | 13.0 | 5000.0 | 38.6 | 118.8 | (0.30, |
| (1-178) | (3-22-1) | | | | | | 0.60) |
| Example | Compound | 4.9 | 12.8 | 5000.0 | 39.2 | 112.9 | (0.31, |
| (1-179) | (3-23-1) | | | | | | 0.61) |
| Example | Compound | 4.9 | 12.7 | 5000.0 | 39.5 | 114.2 | (0.31, |
| (1-180) | (3-24-1) | | | | | | 0.60) |
| Example | Compound | 5.0 | 12.7 | 5000.0 | 39.4 | 138.5 | (0.33, |
| (1-181) | (3-25-1) | | | | | | 0.61) |
| Example | Compound | 4.8 | 13.8 | 5000.0 | 36.3 | 142.0 | (0.32, |
| (1-182) | (3-26-1) | | | | | | 0.61) |
| Example | Compound | 4.8 | 13.8 | 5000.0 | 36.2 | 90.4 | (0.33, |
| (1-183) | (3-27-1) | | | | | | 0.60) |
| Example | Compound | 5.0 | 13.9 | 5000.0 | 35.9 | 138.5 | (0.32, |
| (1-184) | (3-28-1) | | | | | | 0.61) |
| Example | Compound | 4.8 | 13.2 | 5000.0 | 37.8 | 93.3 | (0.31, |
| (1-185) | (3-29-1) | | | | | | 0.60) |
| Example | Compound | 5.0 | 14.2 | 5000.0 | 35.2 | 119.6 | (0.33, |
| (1-186) | (3-30-1) | | | | | | 0.61) |
| Example | Compound | 4.9 | 14.0 | 5000.0 | 35.8 | 113.7 | (0.30, |
| (1-187) | (3-31-1) | | | | | | 0.60) |
| Example | Compound | 4.9 | 13.9 | 5000.0 | 35.9 | 105.4 | (0.31, |
| (1-188) | (3-32-1) | | | | | | 0.61) |
| Example | Compound | 5.0 | 12.8 | 5000.0 | 39.1 | 125.7 | (0.31, |
| (1-189) | (3-33-1) | | | | | | 0.60) |
| Example | Compound | 4.8 | 12.8 | 5000.0 | 39.0 | 140.6 | (0.33, |
| (1-190) | (3-34-1) | | | | | | 0.61) |
| Example | Compound | 5.0 | 13.3 | 5000.0 | 37.7 | 108.9 | (0.32, |
| (1-191) | (3-35-1) | | | | | | 0.61) |
| Example | Compound | 4.8 | 13.3 | 5000.0 | 37.5 | 128.5 | (0.33, |
| (1-192) | (3-36-1) | | | | | | 0.60) |
| Example | Compound | 4.7 | 13.4 | 5000.0 | 37.4 | 104.7 | (0.32, |
| (1-193) | (3-37-1) | | | | | | 0.61) |
| Example | Compound | 5.0 | 13.3 | 5000.0 | 37.6 | 116.8 | (0.31, |
| (1-194) | (3-38-1) | | | | | | 0.60) |
| Example | Compound | 5.0 | 13.1 | 5000.0 | 38.1 | 124.2 | (0.31, |
| (1-195) | (3-39-1) | | | | | | 0.61) |
| Example | Compound | 4.7 | 14.3 | 5000.0 | 35.0 | 90.9 | (0.31, |
| (1-196) | (3-40-1) | | | | | | 0.60) |
| Example | Compound | 4.9 | 12.8 | 5000.0 | 39.2 | 129.5 | (0.33, |
| (1-197) | (3-41-1) | | | | | | 0.61) |
| Example | Compound | 4.9 | 12.8 | 5000.0 | 39.0 | 99.2 | (0.30, |
| (1-198) | (3-42-1) | | | | | | 0.60) |
| Example | Compound | 4.8 | 12.8 | 5000.0 | 39.0 | 118.4 | (0.31, |
| (1-199) | (3-43-1) | | | | | | 0.61) |
| Example | Compound | 4.7 | 14.0 | 5000.0 | 35.8 | 133.3 | (0.31, |
| (1-200) | (3-44-1) | | | | | | 0.60) |
| Example | Compound | 4.7 | 13.5 | 5000.0 | 37.1 | 103.0 | (0.33, |
| (1-201) | (3-45-1) | | | | | | 0.61) |
| Example | Compound | 4.8 | 12.8 | 5000.0 | 38.9 | 148.1 | (0.32, |
| (1-202) | (3-46-1) | | | | | | 0.61) |
| Example | Compound | 4.8 | 13.0 | 5000.0 | 38.4 | 148.3 | (0.33, |
| (1-203) | (3-47-1) | | | | | | 0.61) |
| Example | Compound | 4.9 | 13.7 | 5000.0 | 36.4 | 146.1 | (0.30, |
| (1-204) | (3-48-1) | | | | | | 0.60) |
| Example | Compound | 4.8 | 13.6 | 5000.0 | 36.7 | 102.9 | (0.32, |
| (1-205) | (3-49-1) | | | | | | 0.61) |
| Example | Compound | 4.9 | 14.1 | 5000.0 | 35.4 | 107.6 | (0.31, |
| (1-206) | (3-50-1) | | | | | | 0.60) |
| Example | Compound | 5.0 | 13.1 | 5000.0 | 38.0 | 116.4 | (0.31, |
| (1-207) | (3-51-1) | | | | | | 0.61) |
| Example | Compound | 4.7 | 12.8 | 5000.0 | 38.9 | 125.1 | (0.31, |
| (1-208) | (3-52-1) | | | | | | 0.60) |
| Example | Compound | 4.8 | 13.7 | 5000.0 | 36.5 | 119.3 | (0.33, |
| (1-209) | (3-53-1) | | | | | | 0.61) |
| Example | Compound | 4.8 | 13.4 | 5000.0 | 37.2 | 128.3 | (0.32, |
| (1-210) | (3-54-1) | | | | | | 0.61) |
| Example | Compound | 4.7 | 13.0 | 5000.0 | 38.6 | 116.3 | (0.33, |
| (1-211) | (3-55-1) | | | | | | 0.60) |
| Example | Compound | 4.8 | 13.7 | 5000.0 | 36.4 | 97.9 | (0.32, |
| (1-212) | (3-56-1) | | | | | | 0.61) |
| Example | Compound | 4.8 | 12.7 | 5000.0 | 39.3 | 144.9 | (0.31, |
| (1-213) | (3-57-1) | | | | | | 0.60) |
| Example | Compound | 5.0 | 12.7 | 5000.0 | 39.2 | 112.1 | (0.31, |
| (1-214) | (3-58-1) | | | | | | 0.61) |
| Example | Compound | 4.8 | 13.7 | 5000.0 | 36.5 | 145.6 | (0.31, |
| (1-215) | (3-59-1) | | | | | | 0.60) |
| Example | Compound | 4.8 | 13.9 | 5000.0 | 35.9 | 104.8 | (0.33, |
| (1-216) | (3-60-1) | | | | | | 0.61) |
| Example | Compound | 4.9 | 13.8 | 5000.0 | 36.2 | 106.6 | (0.30, |
| (1-217) | (3-61-1) | | | | | | 0.60) |
| Example | Compound | 5.0 | 12.7 | 5000.0 | 39.5 | 127.2 | (0.31, |
| (1-218) | (3-62-1) | | | | | | 0.61) |
| Example | Compound | 4.9 | 13.2 | 5000.0 | 38.0 | 139.6 | (0.31, |
| (1-219) | (3-63-1) | | | | | | 0.60) |
| Example | Compound | 4.8 | 13.7 | 5000.0 | 36.4 | 111.9 | (0.33, |
| (1-220) | (3-64-1) | | | | | | 0.61) |
| Example | Compound | 4.8 | 13.3 | 5000.0 | 37.7 | 123.4 | (0.32, |
| (1-221) | (3-65-1) | | | | | | 0.61) |
| Example | Compound | 4.8 | 13.2 | 5000.0 | 38.0 | 123.1 | (0.33, |
| (1-222) | (3-66-1) | | | | | | 0.60) |
| Example | Compound | 4.9 | 13.6 | 5000.0 | 36.8 | 145.5 | (0.32, |
| (1-223) | (3-67-1) | | | | | | 0.61) |
| Example | Compound | 5.0 | 12.5 | 5000.0 | 40.0 | 122.5 | (0.31, |
| (1-224) | (3-68-1) | | | | | | 0.60) |
| Example | Compound | 4.8 | 13.0 | 5000.0 | 38.6 | 150.0 | (0.31, |
| (1-225) | (3-69-1) | | | | | | 0.61) |
| Example | Compound | 4.7 | 13.6 | 5000.0 | 36.8 | 92.5 | (0.31, |
| (1-226) | (3-70-1) | | | | | | 0.60) |
| Example | Compound | 5.0 | 14.3 | 5000.0 | 35.0 | 107.2 | (0.33, |
| (1-227) | (3-71-1) | | | | | | 0.61) |
| Example | Compound | 4.9 | 13.2 | 5000.0 | 38.0 | 108.3 | (0.30, |
| (1-228) | (3-72-1) | | | | | | 0.60) |
| Example | Compound | 4.8 | 14.2 | 5000.0 | 35.3 | 122.4 | (0.31, |
| (1-229) | (3-73-1) | | | | | | 0.61) |
| Example | Compound | 5.0 | 14.2 | 5000.0 | 35.2 | 131.7 | (0.31, |
| (1-230) | (3-74-1) | | | | | | 0.60) |
| Example | Compound | 4.9 | 14.2 | 5000.0 | 35.2 | 101.5 | (0.33, |
| (1-231) | (3-75-1) | | | | | | 0.61) |
| Example | Compound | 4.7 | 14.1 | 5000.0 | 35.6 | 108.6 | (0.32, |
| (1-232) | (3-76-1) | | | | | | 0.61) |
| Example | Compound | 5.0 | 12.7 | 5000.0 | 39.5 | 138.2 | (0.33, |
| (1-233) | (3-77-1) | | | | | | 0.60) |
| Example | Compound | 4.9 | 12.6 | 5000.0 | 39.8 | 127.0 | (0.32 |
| (1-234) | (3-78-1) | | | | | | 0.61) |
| Example | Compound | 4.8 | 12.7 | 5000.0 | 39.5 | 130.4 | (0.31, |
| (1-235) | (3-79-1) | | | | | | 0.60) |
| Example | Compound | 4.9 | 13.7 | 5000.0 | 36.5 | 147.5 | (0.33, |
| (1-236) | (3-80-1) | | | | | | 0.61) |
| Example | Compound | 4.9 | 13.4 | 5000.0 | 37.3 | 138.2 | (0.30, |
| (1-237) | (3-81-1) | | | | | | 0.60) |
| Example | Compound | 4.8 | 13.7 | 5000.0 | 36.6 | 133.6 | (0.31, |
| (1-238) | (3-82-1) | | | | | | 0.61) |
| Example | Compound | 5.0 | 12.8 | 5000.0 | 39.0 | 105.9 | (0.31, |
| (1-239) | (3-83-1) | | | | | | 0.60) |
| Example | Compound | 5.0 | 12.5 | 5000.0 | 39.9 | 147.9 | (0.33, |
| (1-240) | (3-84-1) | | | | | | 0.61) |
| Example | Compound | 4.7 | 14.1 | 5000.0 | 35.4 | 130.5 | (0.32, |
| (1-241) | (3-85-1) | | | | | | 0.61) |
| Example | Compound | 4.9 | 14.2 | 5000.0 | 35.1 | 120.7 | (0.33, |
| (1-242) | (3-86-1) | | | | | | 0.60) |
| Example | Compound | 4.8 | 13.9 | 5000.0 | 36.1 | 103.1 | (0.32, |
| (1-243) | (3-87-1) | | | | | | 0.61) |
| Example | Compound | 4.9 | 12.9 | 5000.0 | 38.7 | 133.0 | (0.31, |
| (1-244) | (3-88-1) | | | | | | 0.60) |
| Example | Compound | 4.9 | 14.2 | 5000.0 | 35.2 | 104.9 | (0.31, |
| (1-245) | (3-89-1) | | | | | | 0.61) |
| Example | Compound | 4.8 | 12.6 | 5000.0 | 39.7 | 135.0 | (0.31, |
| (1-246) | (3-90-1) | | | | | | 0.60) |
| Example | Compound | 4.8 | 14.0 | 5000.0 | 35.7 | 105.7 | (0.33, |
| (1-247) | (3-91-1) | | | | | | 0.61) |
| Example | Compound | 4.7 | 12.6 | 5000.0 | 39.7 | 103.6 | (0.30, |
| (1-248) | (3-92-1) | | | | | | 0.60) |
| Example | Compound | 4.8 | 13.4 | 5000.0 | 37.3 | 123.4 | (0.31, |
| (1-249) | (3-93-1) | | | | | | 0.61) |
| Example | Compound | 4.7 | 13.6 | 5000.0 | 36.7 | 96.8 | (0.31, |
| (1-250) | (3-94-1) | | | | | | 0.60) |
| Example | Compound | 4.9 | 13.6 | 5000.0 | 36.8 | 114.3 | (0.33, |
| (1-251) | (3-95-1) | | | | | | 0.61) |
| Example | Compound | 4.9 | 12.9 | 5000.0 | 38.9 | 93.8 | (0.32, |
| (1-252) | (3-96-1) | | | | | | 0.61) |
| Example | Compound | 4.8 | 13.6 | 5000.0 | 36.7 | 97.5 | (0.33, |
| (1-253) | (3-97-1) | | | | | | 0.61) |
| Example | Compound | 4.7 | 14.0 | 5000.0 | 35.7 | 119.5 | (0.30, |
| (1-254) | (3-98-1) | | | | | | 0.60) |
| Example | Compound | 4.9 | 14.1 | 5000.0 | 35.5 | 116.2 | (0.32, |
| (1-255) | (3-99-1) | | | | | | 0.61) |
| Example | Compound | 4.7 | 13.3 | 5000.0 | 37.5 | 101.8 | (0.31, |
| (1-256) | (3-100-1) | | | | | | 0.60) |
| Example | Compound | 4.8 | 12.8 | 5000.0 | 39.1 | 125.7 | (0.30, |
| (1-257) | (3-101-1) | | | | | | 0.60) |
| Example | Compound | 5.0 | 12.8 | 5000.0 | 39.0 | 140.2 | (0.31, |
| (1-258) | (3-102-1) | | | | | | 0.61) |
| Example | Compound | 5.0 | 12.8 | 5000.0 | 39.1 | 134.5 | (0.31, |
| (1-259) | (3-103-1) | | | | | | 0.60) |
| Example | Compound | 4.9 | 12.9 | 5000.0 | 38.7 | 93.4 | (0.33, |
| (1-260) | (3-104-1) | | | | | | 0.61) |
| Example | Compound | 4.7 | 13.2 | 5000.0 | 37.8 | 120.1 | (0.32, |
| (1-261) | (3-105-1) | | | | | | 0.61) |
| Example | Compound | 5.0 | 13.4 | 5000.0 | 37.2 | 98.6 | (0.33, |
| (1-262) | (3-106-1) | | | | | | 0.60) |
| Example | Compound | 4.8 | 13.8 | 5000.0 | 36.3 | 99.5 | (0.32, |
| (1-263) | (3-107-1) | | | | | | 0.61) |
| Example | Compound | 5.0 | 12.6 | 5000.0 | 39.8 | 92.0 | (0.31, |
| (1-264) | (3-108-1) | | | | | | 0.60) |
| Example | Compound | 5.0 | 12.7 | 5000.0 | 39.3 | 98.1 | (0.31, |
| (1-265) | (3-109-1) | | | | | | 0.61) |
| Example | Compound | 5.0 | 13.3 | 5000.0 | 37.6 | 115.8 | (0.31, |
| (1-266) | (3-110-1) | | | | | | 0.60) |
| Example | Compound | 4.9 | 12.7 | 5000.0 | 39.4 | 114.0 | (0.33, |
| (1-267) | (3-111-1) | | | | | | 0.61) |
| Example | Compound | 4.7 | 12.5 | 5000.0 | 39.9 | 112.6 | (0.30, |
| (1-268) | (3-112-1) | | | | | | 0.60) |
| Example | Compound | 4.8 | 13.3 | 5000.0 | 37.7 | 132.6 | (0.31, |
| (1-269) | (3-113-1) | | | | | | 0.61) |
| Example | Compound | 4.8 | 12.7 | 5000.0 | 39.2 | 118.4 | (0.31, |
| (1-270) | (3-114-1) | | | | | | 0.60) |
| Example | Compound | 4.9 | 12.6 | 5000.0 | 39.7 | 147.2 | (0.33, |
| (1-271) | (3-115-1) | | | | | | 0.61) |
| Example | Compound | 5.0 | 13.0 | 5000.0 | 38.6 | 149.5 | (0.32, |
| (1-272) | (3-116-1) | | | | | | 0.61) |
| Example | Compound | 5.0 | 13.0 | 5000.0 | 38.4 | 91.1 | (0.33, |
| (1-273) | (3-117-1) | | | | | | 0.60) |
| Example | Compound | 4.8 | 14.0 | 5000.0 | 35.7 | 108.2 | (0.32, |
| (1-274) | (3-118-1) | | | | | | 0.61) |
| Example | Compound | 4.9 | 12.8 | 5000.0 | 39.2 | 114.3 | (0.31, |
| (1-275) | (3-119-1) | | | | | | 0.60) |
| Example | Compound | 4.9 | 13.9 | 5000.0 | 35.8 | 114.7 | (0.31, |
| (1-276) | (3-120-1) | | | | | | 0.61) |
| Example | Compound | 4.9 | 14.1 | 5000.0 | 35.4 | 122.9 | (0.31, |
| (1-277) | (3-121-1) | | | | | | 0.60) |
| Example | Compound | 4.7 | 13.1 | 5000.0 | 38.2 | 126.3 | (0.33, |
| (1-278) | (3-122-1) | | | | | | 0.61) |
| Example | Compound | 4.8 | 12.6 | 5000.0 | 39.7 | 100.0 | (0.30, |
| (1-279) | (3-123-1) | | | | | | 0.60) |
| Example | Compound | 4.9 | 14.2 | 5000.0 | 35.1 | 149.9 | (0.31, |
| (1-280) | (3-124-1) | | | | | | 0.61) |
| Example | Compound | 4.8 | 12.8 | 5000.0 | 39.0 | 105.4 | (0.31, |
| (1-281) | (3-125-1) | | | | | | 0.60) |
| Example | Compound | 4.8 | 14.1 | 5000.0 | 35.4 | 142.9 | (0.33, |
| (1-282) | (3-126-1) | | | | | | 0.61) |
| Example | Compound | 4.7 | 12.8 | 5000.0 | 39.0 | 115.2 | (0.32, |
| (1-283) | (3-127-1) | | | | | | 0.61) |
| Example | Compound | 4.9 | 13.3 | 5000.0 | 37.5 | 120.9 | (0.33, |
| (1-284) | (3-128-1) | | | | | | 0.60) |
| Example | Compound | 4.8 | 15.1 | 5000.0 | 33.1 | 132.0 | (0.31, |
| (1-285) | (4-1-1) | | | | | | 0.61) |
| Example | Compound | 5.0 | 15.9 | 5000.0 | 31.5 | 123.8 | (0.31, |
| (1-286) | (4-2-1) | | | | | | 0.60) |
| Example | Compound | 4.8 | 15.2 | 5000.0 | 32.9 | 114.1 | (0.33, |
| (1-287) | (4-3-1) | | | | | | 0.61) |
| Example | Compound | 4.7 | 15.2 | 5000.0 | 32.9 | 103.3 | (0.32, |
| (1-288) | (4-4-1) | | | | | | 0.61) |
| Example | Compound | 4.8 | 15.2 | 5000.0 | 32.8 | 100.1 | (0.33, |
| (1-289) | (4-5-1) | | | | | | 0.60) |
| Example | Compound | 4.9 | 14.9 | 5000.0 | 33.5 | 129.1 | (0.32, |
| (1-290) | (4-6-1) | | | | | | 0.61) |
| Example | Compound | 4.8 | 14.7 | 5000.0 | 34.1 | 96.8 | (0.31, |
| (1-291) | (4-7-1) | | | | | | 0.60) |
| Example | Compound | 5.0 | 15.1 | 5000.0 | 33.0 | 123.5 | (0.31, |
| (1-292) | (4-8-1) | | | | | | 0.61) |
| Example | Compound | 5.0 | 16.5 | 5000.0 | 30.3 | 125.7 | (0.31, |
| (1-293) | (4-9-1) | | | | | | 0.60) |
| Example | Compound | 4.8 | 15.0 | 5000.0 | 33.2 | 149.5 | (0.33, |
| (1-294) | (4-10-1) | | | | | | 0.61) |
| Example | Compound | 4.8 | 14.9 | 5000.0 | 33.4 | 95.9 | (0.30, |
| (1-295) | (4-11-1) | | | | | | 0.60) |
| Example | Compound | 4.7 | 15.8 | 5000.0 | 31.6 | 113.8 | (0.31, |
| (1-296) | (4-12-1) | | | | | | 0.61) |
| Example | Compound | 4.7 | 14.6 | 5000.0 | 34.2 | 122.5 | (0.31, |
| (1-297) | (4-13-1) | | | | | | 0.60) |
| Example | Compound | 4.8 | 14.5 | 5000.0 | 34.5 | 115.5 | (0.33, |
| (1-298) | (4-14-1) | | | | | | 0.61) |
| Example | Compound | 4.9 | 15.2 | 5000.0 | 32.8 | 148.6 | (0.32, |
| (1-299) | (4-15-1) | | | | | | 0.61) |
| Example | Compound | 5.0 | 16.0 | 5000.0 | 31.3 | 91.2 | (0.33, |
| (1-300) | (4-16-1) | | | | | | 0.60) |
| Example | Compound | 4.8 | 14.9 | 5000.0 | 33.5 | 137.2 | (0.32, |
| (1-301) | (4-17-1) | | | | | | 0.61) |
| Example | Compound | 4.7 | 15.5 | 5000.0 | 32.2 | 90.3 | (0.31, |
| (1-302) | (4-18-1) | | | | | | 0.60) |
| Example | Compound | 4.9 | 14.3 | 5000.0 | 34.9 | 97.1 | (0.31, |
| (1-303) | (4-19-1) | | | | | | 0.61) |
| Example | Compound | 4.9 | 14.9 | 5000.0 | 33.6 | 125.5 | (0.31, |
| (1-304) | (4-20-1) | | | | | | 0.60) |
| Example | Compound | 4.9 | 14.4 | 5000.0 | 34.7 | 105.7 | (0.33, |
| (1-305) | (4-21-1) | | | | | | 0.61) |
| Example | Compound | 4.9 | 16.1 | 5000.0 | 31.0 | 107.7 | (0.30, |
| (1-306) | (4-22-1) | | | | | | 0.60) |
| Example | Compound | 4.7 | 14.7 | 5000.0 | 34.0 | 145.0 | (0.31, |
| (1-307) | (4-23-1) | | | | | | 0.61) |
| Example | Compound | 5.0 | 15.8 | 5000.0 | 31.6 | 98.3 | (0.31, |
| (1-308) | (4-24-1) | | | | | | 0.60) |
| Example | Compound | 4.8 | 14.6 | 5000.0 | 34.2 | 98.9 | (0.33, |
| (1-309) | (4-25-1) | | | | | | 0.61) |
| Example | Compound | 4.8 | 15.4 | 5000.0 | 32.4 | 95.3 | (0.32, |
| (1-310) | (4-26-1) | | | | | | 0.61) |
| Example | Compound | 4.7 | 15.8 | 5000.0 | 31.6 | 90.2 | (0.33, |
| (1-311) | (4-27-1) | | | | | | 0.60) |
| Example | Compound | 4.7 | 14.4 | 5000.0 | 34.7 | 146.4 | (0.32, |
| (1-312) | (4-28-1) | | | | | | 0.61) |

### □. Manufacture and Test of Red Organic Light Emitting Element (Phosphorescent Host)

### Example 1-313 Red Organic Light Emitting Element (Phosphorescent Host)

An organic electronic light emitting element was manufactured by an ordinary method using the compound obtained through synthesis as a light emitting host material for a light emitting layer. First, a film of N¹-(naphthalen-2-yl)-N⁴,N⁴-bis(4-(naphthalen-2-yl(phenyl)amino)phenyl)-N¹-phenylbenzene-1,4-diamine (hereinafter, abbreviated as “2-TNATA”) as a hole transport compound was vacuum-deposited on an ITO layer (anode) formed on a galas substrate to form a hole injection layer with a thickness 60 nm, and then, 4,4-bis[N-(1-naphthyl)-N-phenylamino]biphenyl (hereinafter, abbreviated as “-NPD”) as a hole transport compound was vacuum-deposited on the hole injection layer to form a hole transport layer with a thickness of 60 nm. Then, a light emitting layer with a thickness of 30 nm was deposited on the hole transport layer by doping an upper portion of the hole transport layer with compound 2-41-1 of the present invention as a host material and (piq)₂Ir(acac) [bis-(1-phenylisoquinolyl)iridium(III)acetylacetonate] as a dopant material at a weight ratio of 95:5. Then, (1.1′-bisphenyl)-4-olato)bis(2-methyl-8-quinolinolato)aluminum (hereinafter, abbreviated as “BAlq”) was vacuum-deposited with a thickness of 10 nm for a hole blocking layer, and tris(8-quinolinol)aluminum (hereinafter, abbreviated as “Alq3”) was formed with a thickness of 40 nm for an electron transport layer. Thereafter, LiF as halogenated alkali metal was deposited with a thickness of 0.2 nm for an electron injection layer, and then Al was deposited with a thickness of 150 nm to be used as a cathode. In this way, an organic electronic light emitting element was manufactured.

### Example 1-314 to Example 1-336 Red Organic Light Emitting Element (Phosphorescent Host)

An organic electronic light emitting element was manufactured by the same method as in Example 1-313 except that, instead of compound 2-41-1 of the present invention, one of compounds 2-42-1 to 2-52-1 and 3-41-1 to 3-52-1 listed on table 6 was used as a phosphorescent host material for a light emitting layer.

### Comparative Example 1-5

An organic electronic light emitting element was manufactured by the same method as in Example 1-313 except that, instead of compound 2-41-1 of the present invention, comparative compound A [4,4′-N,N′-dicarbazole-biphenyl (CBP)] above was used as a phosphorescent host material for a light emitting layer.

### Comparative Example 1-6

An organic electronic light emitting element was manufactured by the same method as in Example 1-313 except that, instead of compound 2-41-1 of the present invention, comparative compound B above was used as a phosphorescent host material for a light emitting layer.

### Comparative Example 1-7

An organic electronic light emitting element was manufactured by the same method as in Example 1-313 except that, instead of compound 2-41-1 of the present invention, comparative compound C above was used as a phosphorescent host material for a light emitting layer.

### Comparative Example 1-8

An organic electronic light emitting element was manufactured by the same method as in Example 1-313 except that, instead of compound 2-41-1 of the present invention, comparative compound D above was used as a phosphorescent host material for a light emitting layer.

A forward bias DC voltage was applied to the organic electronic light emitting elements manufactured in Examples 1-313 to 1-336 and Comparative Examples 1-5 to 1-8 to measure electro-luminescence (EL) characteristics thereof by PR-650 (Photoresearch), and the T95 lifetime was measured by lifetime measuring equipments (Mcscience) at reference brightness of 2500 cd/m². Table 1-6 below shows the manufacture of elements and evaluation results thereof.

TABLE 1-6

| | | | Current | Brightness | | Lifetime | CIE |
|---|---|---|---|---|---|---|---|
| | Compound | Voltage | Density | (cd/m2) | Efficiency | T (95) | (x, y) |
| Comparative | Compound | 6.0 | 38.5 | 2500.0 | 6.5 | 70.6 | (0.31, 0.60) |
| Example | (A) | | | | | | |
| (1-5) | | | | | | | |
| Comparative | Compound | 5.7 | 31.4 | 2500.0 | 8.0 | 81.0 | (0.31, 0.61) |
| Example | (B) | | | | | | |
| (1-6) | | | | | | | |
| Comparative | Compound | 5.8 | 35.5 | 2500.0 | 7.0 | 80.2 | (0.31, 0.60) |
| Example | (C) | | | | | | |
| (1-7) | | | | | | | |
| Comparative | Compound | 5.9 | 35.1 | 2500.0 | 7.1 | 87.7 | (0.33, 0.61) |
| Example | (D) | | | | | | |
| (1-8) | | | | | | | |
| Example | Compound | 5.1 | 29.0 | 2500.0 | 8.6 | 138.0 | (0.30, 0.60) |
| (1-313) | (2-41-1) | | | | | | |
| Example | Compound | 5.2 | 28.5 | 2500.0 | 8.8 | 123.2 | (0.31, 0.61) |
| (1-314) | (2-42-1) | | | | | | |
| Example | Compound | 5.4 | 28.3 | 2500.0 | 8.8 | 130.6 | (0.31, 0.60) |
| (1-315) | (2-43-1) | | | | | | |
| Example | Compound | 5.5 | 31.0 | 2500.0 | 8.1 | 129.6 | (0.33, 0.61) |
| (1-316) | (2-44-1) | | | | | | |
| Example | Compound | 5.3 | 30.3 | 2500.0 | 8.2 | 101.0 | (0.32, 0.61) |
| (1-317) | (2-45-1) | | | | | | |
| Example | Compound | 5.1 | 31.2 | 2500.0 | 8.0 | 95.1 | (0.33, 0.60) |
| (1-318) | (2-46-1) | | | | | | |
| Example | Compound | 5.4 | 29.7 | 2500.0 | 8.4 | 107.6 | (0.32, 0.61) |
| (1-319) | (2-47-1) | | | | | | |
| Example | Compound | 5.0 | 31.2 | 2500.0 | 8.0 | 110.2 | (0.31, 0.60) |
| (1-320) | (2-48-1) | | | | | | |
| Example | Compound | 5.1 | 29.6 | 2500.0 | 8.4 | 107.7 | (0.31, 0.61) |
| (1-321) | (2-49-1) | | | | | | |
| Example | Compound | 5.4 | 30.0 | 2500.0 | 8.3 | 100.2 | (0.31, 0.60) |
| (1-322) | (2-50-1) | | | | | | |
| Example | Compound | 5.1 | 29.9 | 2500.0 | 8.4 | 131.7 | (0.33, 0.61) |
| (1-323) | (2-51-1) | | | | | | |
| Example | Compound | 5.3 | 27.9 | 2500.0 | 9.0 | 120.8 | (0.30, 0.60) |
| (1-324) | (2-52-1) | | | | | | |
| Example | Compound | 5.5 | 28.6 | 2500.0 | 8.7 | 100.2 | (0.31, 0.61) |
| (1-325) | (3-41-1) | | | | | | |
| Example | Compound | 5.5 | 30.7 | 2500.0 | 8.1 | 94.0 | (0.31, 0.60) |
| (1-326) | (3-42-1) | | | | | | |
| Example | Compound | 5.3 | 29.5 | 2500.0 | 8.5 | 115.5 | (0.33, 0.61) |
| (1-327) | (3-43-1) | | | | | | |
| Example | Compound | 5.4 | 28.9 | 2500.0 | 8.6 | 105.3 | (0.32, 0.61) |
| (1-328) | (3-44-1) | | | | | | |
| Example | Compound | 5.1 | 28.8 | 2500.0 | 8.7 | 121.3 | (0.33, 0.60) |
| (1-329) | (3-45-1) | | | | | | |
| Example | Compound | 5.1 | 29.9 | 2500.0 | 8.4 | 115.9 | (0.31, 0.60) |
| (1-330) | (3-46-1) | | | | | | |
| Example | Compound | 5.5 | 28.6 | 2500.0 | 8.8 | 142.2 | (0.31, 0.61) |
| (1-331) | (3-47-1) | | | | | | |
| Example | Compound | 5.4 | 29.1 | 2500.0 | 8.6 | 99.5 | (0.31, 0.60) |
| (1-332) | (3-48-1) | | | | | | |
| Example | Compound | 5.3 | 27.9 | 2500.0 | 8.9 | 95.7 | (0.33, 0.61) |
| (1-333) | (3-49-1) | | | | | | |
| Example | Compound | 5.2 | 30.1 | 2500.0 | 8.3 | 144.3 | (0.30, 0.60) |
| (1-334) | (3-50-1) | | | | | | |
| Example | Compound | 5.1 | 30.0 | 2500.0 | 8.3 | 149.2 | (0.31, 0.61) |
| (1-335) | (3-51-1) | | | | | | |
| Example | Compound | 5.5 | 28.1 | 2500.0 | 8.9 | 139.7 | (0.31, 0.60) |
| (1-336) | (3-52-1) | | | | | | |

As can be seen from the results on table 1-5 and table 1-6, the organic electronic light emitting elements using the materials for the organic electronic light emitting element of the present invention as a phosphorescent host showed a low driving voltage, high light emitting efficiency, and a long lifetime.

In other words, comparative compounds B, C, and D having bis-carbazole as a core showed excellent element results compared with comparative compound A, which is CBP generally used as a host material, and the compounds of the present invention having carbazole linked to carboline showed the best results in view of a driving voltage, efficiency, and a lifetime, compared with comparative compounds B, C, and D.

The compound according to the present invention has a bipolar since it is composed of carbazole and carboline. Therefore, it is considered that the compounds of the present invention can raise the charge balance in the light emitting layer compared with those in comparative compounds B, C, and D, leading to an increase in efficiency, and shows less hole accumulation in the light emitting layer compared with comparative compounds B, C, and D, leading to a long lifetime (In the driving of OLED, holes generally have 1000-fold higher mobility than electrons).

In addition, the compounds according to the present invention have similar T1 values to comparative compounds B, C, and D, but show lower LUMO values, and resultantly, it is considered that the compounds of the present invention may easily receive electrons from the electron transport layer, leading to a low driving voltage and excellent thermal stability (thermal damage due to a high driving voltage).

In addition, the characteristics of elements have been described in view of a light emitting layer from the foregoing evaluation results of the manufacture of elements, but the materials ordinarily used for a light emitting layer may be used alone or in a mixture with other materials, for the foregoing organic material layer for an organic electronic element, such as an electron transport layer, an electron injection layer, a hole injection layer, a hole transport layer, and an auxiliary light emitting layer. Therefore, for the foregoing reasons, the compounds of the present invention may be used alone or in a mixture with other materials, for the other layers for the organic material layer excluding the light emitting layer, for example, an electron transport layer, an electron injection layer, a hole injection layer, a hole transport layer, and an auxiliary light emitting layer.

### Example 2

The compound according to an aspect of the present invention is represented by Formula 2-1 below.

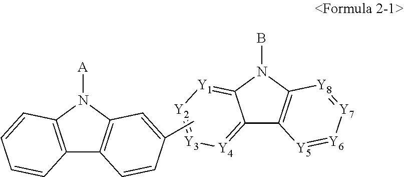

In Formula 2-1,

A and B each may be independently selected from the group consisting of a C₆-C₆₀ aryl group, a fluorenyl group, a C₂-C₆₀ heterocyclic group containing at least one heteroatom of O, N, S, Si, and P, a fused ring group of a C₃-C₆₀ aliphatic group and a C₆-C₆₀ aromatic group, a C₁-C₅₀ alkyl group, a C₂-C₂₀ alkenyl group, a C₂-C₂₀ alkynyl group, C₁-C₃₀ alkoxyl group, a C₆-C₃₀ aryloxy group, and -L′-N(Rₐ) (R_{b}).

L′ may be selected from the group consisting of a single bond, a C₆-C₆₀ arylene group, a fluorenyl group, a fused ring group of a C₃-C₆o aliphatic group and a C₆-C₆₀ aromatic group, and a C₂-C₆₀ heterocyclic group.

Rₐ and R_{b} each may be independently selected from the group consisting of a C₆-C₆₀ aryl group, a fluorenylene group, a fused ring group of a C₃-C₆₀ aliphatic group and a C₆-C₆₀ aromatic group, and a C₂-C₆₀ heterocyclic group containing at least one heteroatom of O, N, S, Si, and P.

Y₁ to Y₈ each may be independently CR or N, and at least one of Y₁ to Y₈ may be N.

At least one of R's may be linked to adjacent carbazole, and R that is not linked thereto may be hydrogen.

For example, when A, B, L′, Rₐ, and R_{b} are an aryl group, A, B, L′, Rₐ, and R_{b} each may be independently a phenyl group, a biphenyl group, a naphthyl group, or the like.

the aryl group, fluorenyl group, heterocyclic group, fused ring group, alkyl group, alkenyl group, alkoxyl group, aryloxy group, arylene group, and fluorenylene group each may be substituted with at least one substituent selected from the group consisting of deuterium, halogen, a silane group, a siloxane group, a boron group, a germanium group, a cyano group, a nitro group, a C₁-C₂₀ alkylthio group, a C₁-C₂₀ alkoxyl group, a C₁-C₂₀ alkyl group, a C₂-C₂₀ alkenyl group, a C₂-C₂₀ alkynyl group, a C₆-C₂₀ aryl group, a C₆-C₂₀ aryl group substituted with deuterium, a fluorenyl group, a C₂-C₂₀ heterocyclic group, a C₃-C₂₀ cycloalkyl group, a C₇-C₂₀ arylalkyl group, and a C₈-C₂₀ arylalkenyl group.

Here, the aryl group may be an aryl group having 6-60 carbon atoms, preferably 6-40 carbon atoms, and more preferably 6-30 carbon atoms;

the heterocyclic group may be a heterocyclic group having 2-60 carbon atoms, preferably 2-30 carbon atoms, and more preferably 2-20 carbon atoms;

the arylene group may be an arylene group having 6-60 carbon atoms, preferably 6-30 carbon atoms, and more preferably 6-20 carbon atoms; and

the alkyl group may be an alkyl group having 1-50 carbon atoms, preferably 1-30 carbon atoms, more preferably 1-20 carbon atoms, and especially preferably 1-10 carbon atoms.

Specifically, the compound represented by Formula 2-1 above may be expressed by one of the following compounds.

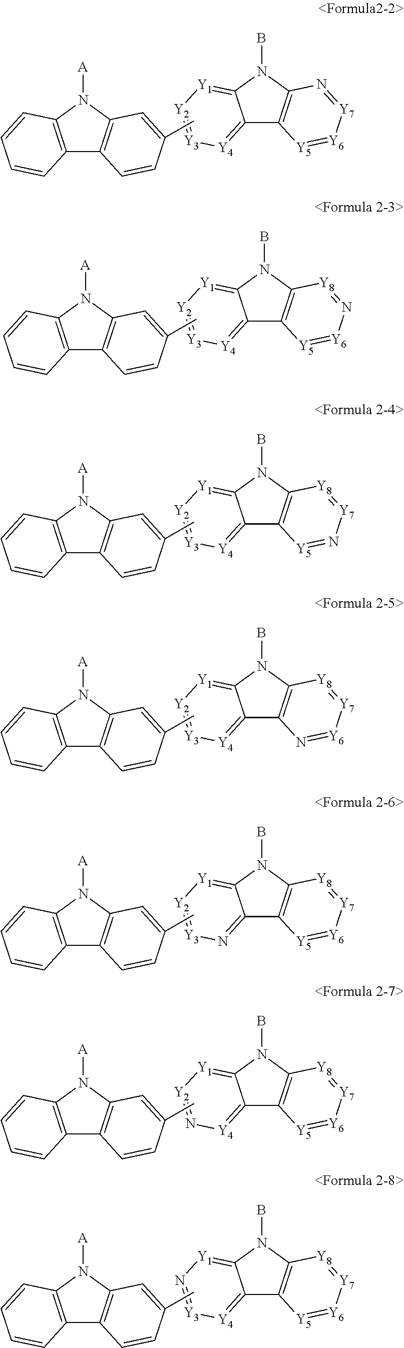

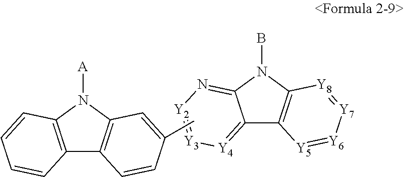

In Formulas 2-2 to 2-9,

Y₁ to Y₈ and A and B may be identical Y₁ to Y₈ and A and B defined in Formula 2-1.

More specifically, the compounds represented by Formula 2-1 may be one of the following compounds.

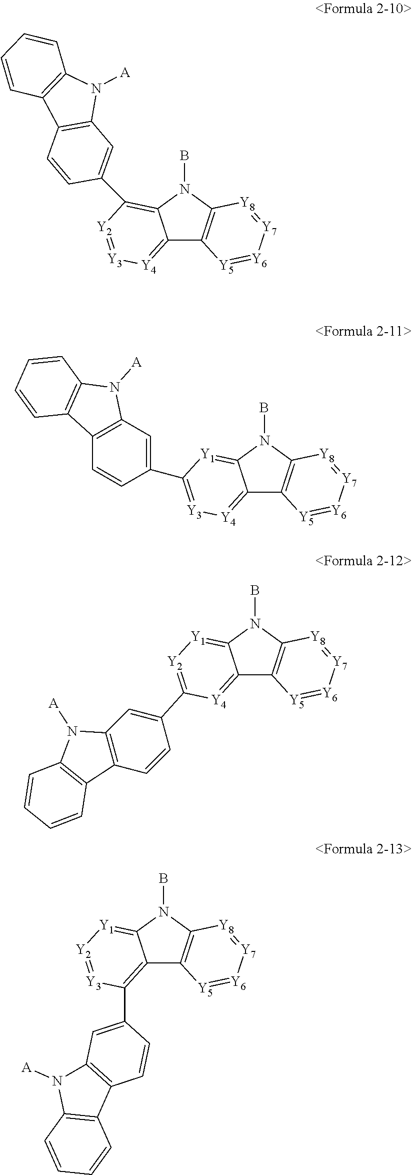

In Formulas 2-10 to 2-13,

Y₁ to Ye each may be independently CH or N, and at least one thereof is N, and A and B may be identical A and B defined in Formula 2-1.

More specifically, the compounds represented by Formulas 2-1 to 2-13 may be one of the following compounds.

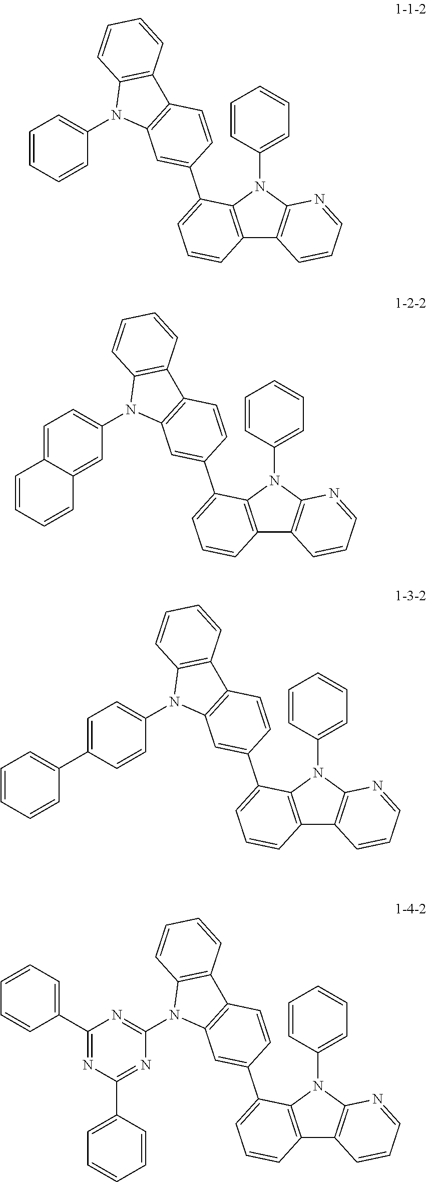 [SMILES: c1ccc(-n2c3ccccc3c3ccc(-c4cccc5c6cccnc6n(-c6ccccc6)c45)cc32)cc1 ; c1ccc(-n2c3ncccc3c3cccc(-c4ccc5c6ccccc6n(-c6ccc7ccccc7c6)c5c4)c32)cc1 ; c1ccc(-c2ccc(-n3c4ccccc4c4ccc(-c5cccc6c7cccnc7n(-c7ccccc7)c56)cc43)cc2)cc1 ; c1ccc(-c2nc(-c3ccccc3)nc(-n3c4ccccc4c4ccc(-c5cccc6c7cccnc7n(-c7ccccc7)c56)cc43)n2)cc1]

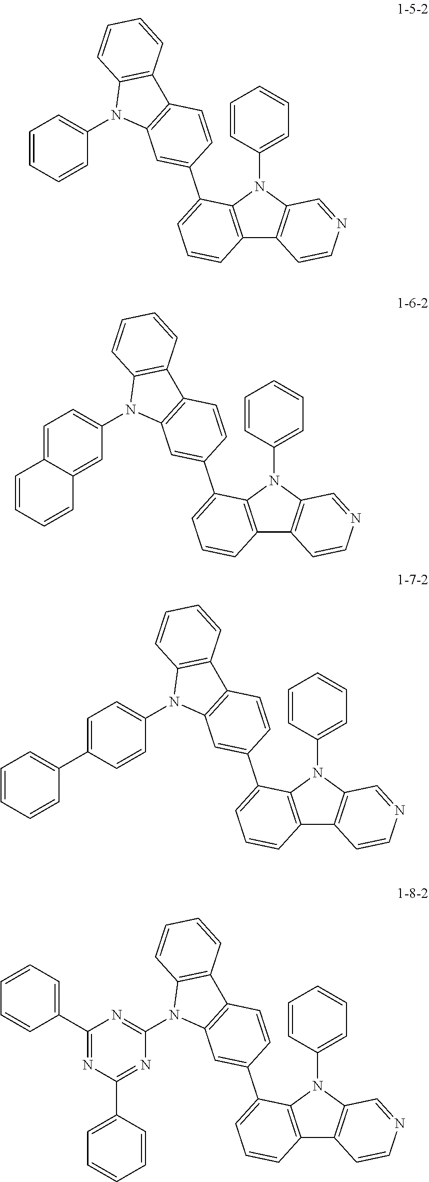 [SMILES: c1ccc(-n2c3ccccc3c3ccc(-c4cccc5c6ccncc6n(-c6ccccc6)c45)cc32)cc1 ; c1ccc(-n2c3cnccc3c3cccc(-c4ccc5c6ccccc6n(-c6ccc7ccccc7c6)c5c4)c32)cc1 ; c1ccc(-c2ccc(-n3c4ccccc4c4ccc(-c5cccc6c7ccncc7n(-c7ccccc7)c56)cc43)cc2)cc1 ; c1ccc(-c2nc(-c3ccccc3)nc(-n3c4ccccc4c4ccc(-c5cccc6c7ccncc7n(-c7ccccc7)c56)cc43)n2)cc1]

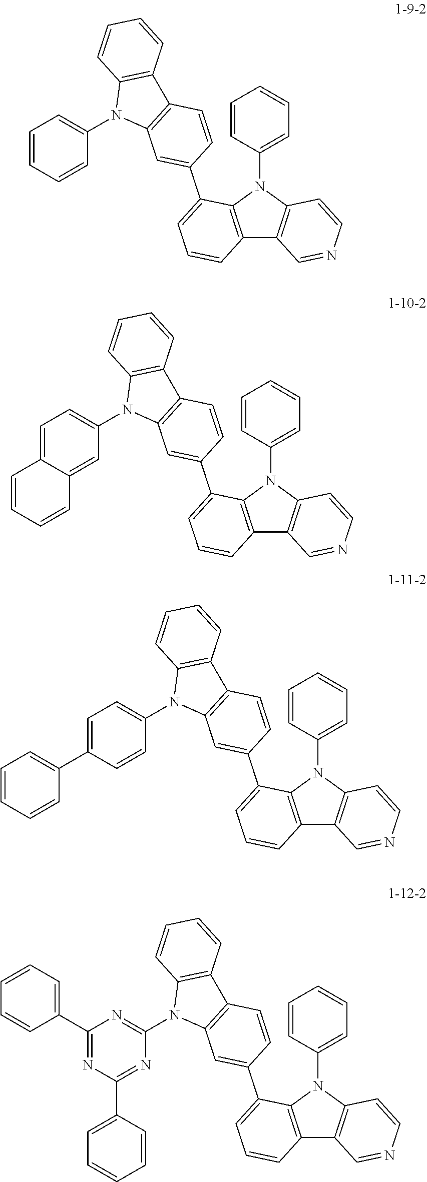 [SMILES: c1ccc(-n2c3ccccc3c3ccc(-c4cccc5c6cnccc6n(-c6ccccc6)c45)cc32)cc1 ; c1ccc(-n2c3ccncc3c3cccc(-c4ccc5c6ccccc6n(-c6ccc7ccccc7c6)c5c4)c32)cc1 ; c1ccc(-c2ccc(-n3c4ccccc4c4ccc(-c5cccc6c7cnccc7n(-c7ccccc7)c56)cc43)cc2)cc1 ; c1ccc(-c2nc(-c3ccccc3)nc(-n3c4ccccc4c4ccc(-c5cccc6c7cnccc7n(-c7ccccc7)c56)cc43)n2)cc1]

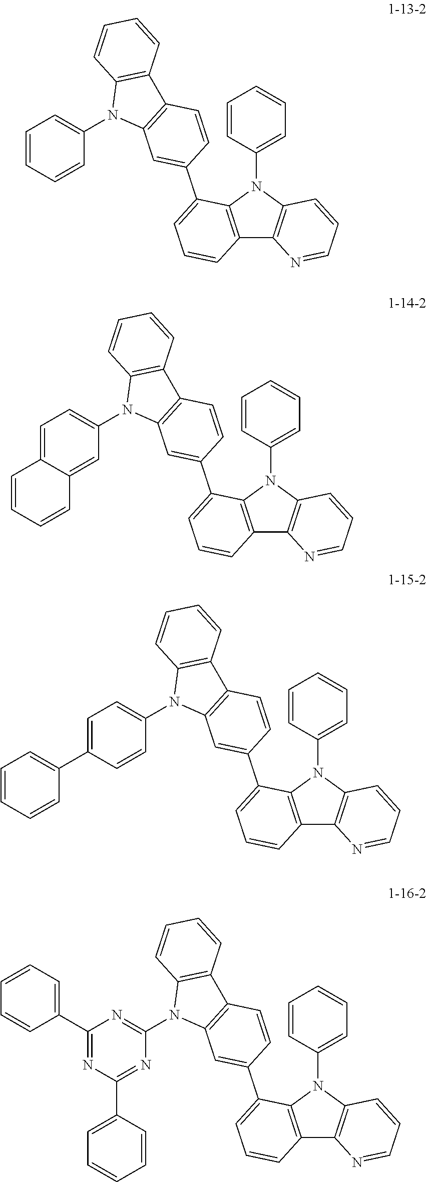 [SMILES: c1ccc(-n2c3ccccc3c3ccc(-c4cccc5c6ncccc6n(-c6ccccc6)c45)cc32)cc1 ; c1ccc(-n2c3cccnc3c3cccc(-c4ccc5c6ccccc6n(-c6ccc7ccccc7c6)c5c4)c32)cc1 ; c1ccc(-c2ccc(-n3c4ccccc4c4ccc(-c5cccc6c7ncccc7n(-c7ccccc7)c56)cc43)cc2)cc1 ; c1ccc(-c2nc(-c3ccccc3)nc(-n3c4ccccc4c4ccc(-c5cccc6c7ncccc7n(-c7ccccc7)c56)cc43)n2)cc1]

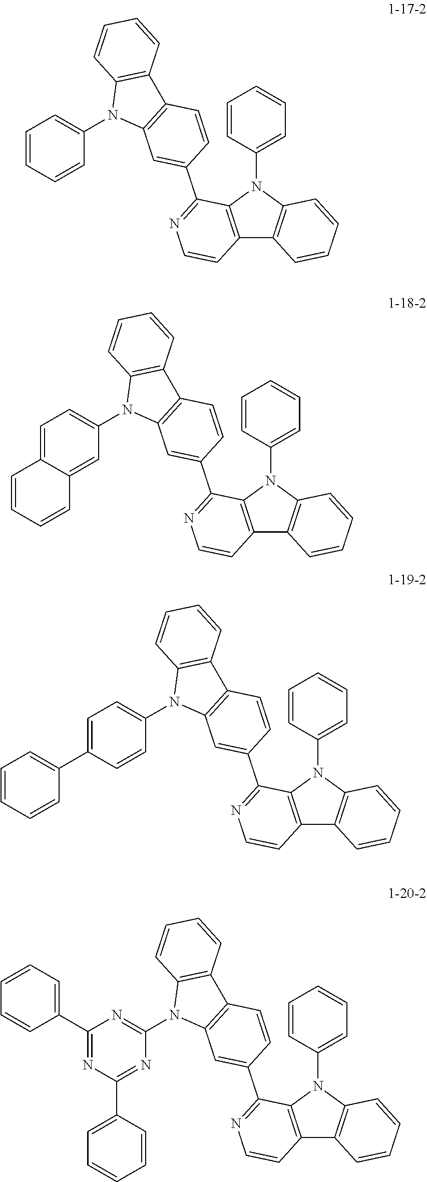 [SMILES: c1ccc(-n2c3ccccc3c3ccc(-c4nccc5c6ccccc6n(-c6ccccc6)c45)cc32)cc1 ; c1ccc(-n2c3ccccc3c3ccnc(-c4ccc5c6ccccc6n(-c6ccc7ccccc7c6)c5c4)c32)cc1 ; c1ccc(-c2ccc(-n3c4ccccc4c4ccc(-c5nccc6c7ccccc7n(-c7ccccc7)c56)cc43)cc2)cc1 ; c1ccc(-c2nc(-c3ccccc3)nc(-n3c4ccccc4c4ccc(-c5nccc6c7ccccc7n(-c7ccccc7)c56)cc43)n2)cc1]

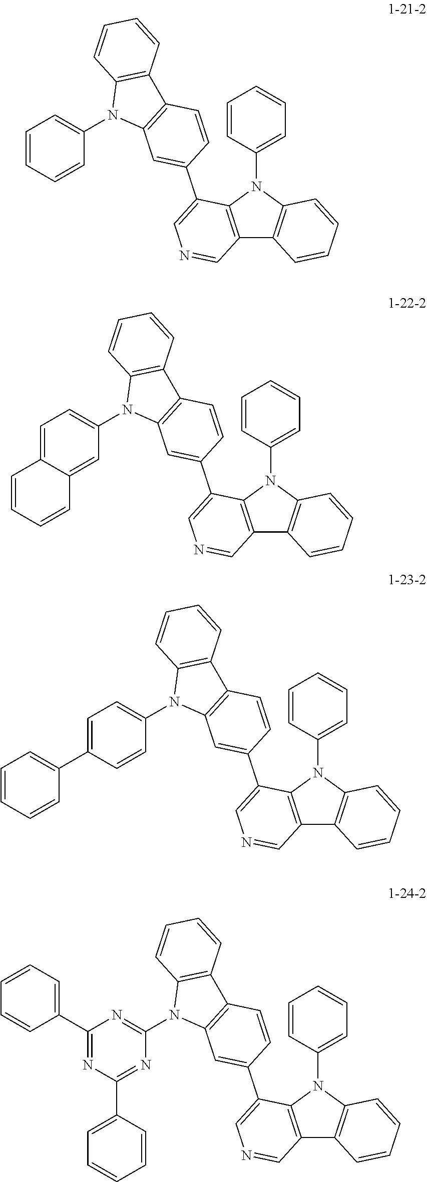 [SMILES: c1ccc(-n2c3ccccc3c3ccc(-c4cncc5c6ccccc6n(-c6ccccc6)c45)cc32)cc1 ; c1ccc(-n2c3ccccc3c3cncc(-c4ccc5c6ccccc6n(-c6ccc7ccccc7c6)c5c4)c32)cc1 ; c1ccc(-c2ccc(-n3c4ccccc4c4ccc(-c5cncc6c7ccccc7n(-c7ccccc7)c56)cc43)cc2)cc1 ; c1ccc(-c2nc(-c3ccccc3)nc(-n3c4ccccc4c4ccc(-c5cncc6c7ccccc7n(-c7ccccc7)c56)cc43)n2)cc1]

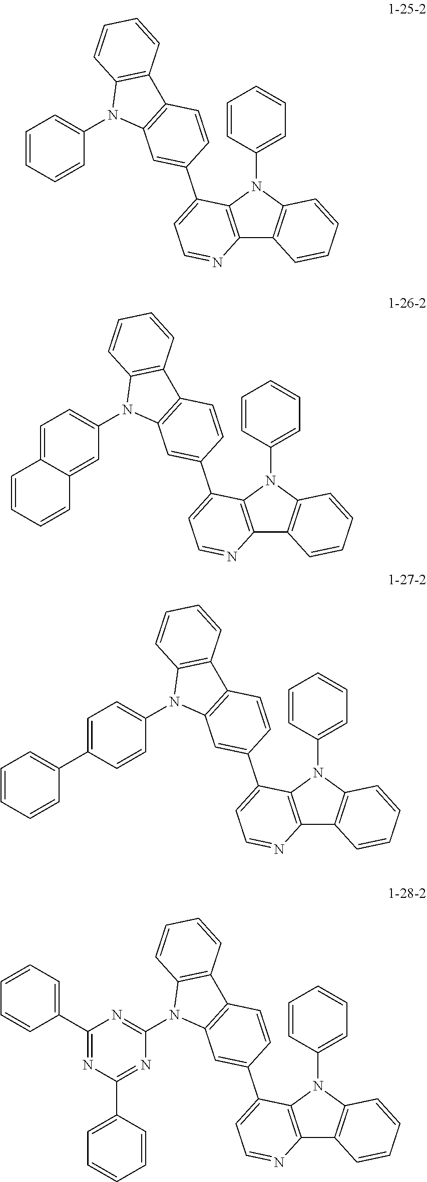 [SMILES: c1ccc(-n2c3ccccc3c3ccc(-c4ccnc5c6ccccc6n(-c6ccccc6)c45)cc32)cc1 ; c1ccc(-n2c3ccccc3c3nccc(-c4ccc5c6ccccc6n(-c6ccc7ccccc7c6)c5c4)c32)cc1 ; c1ccc(-c2ccc(-n3c4ccccc4c4ccc(-c5ccnc6c7ccccc7n(-c7ccccc7)c56)cc43)cc2)cc1 ; c1ccc(-c2nc(-c3ccccc3)nc(-n3c4ccccc4c4ccc(-c5ccnc6c7ccccc7n(-c7ccccc7)c56)cc43)n2)cc1]

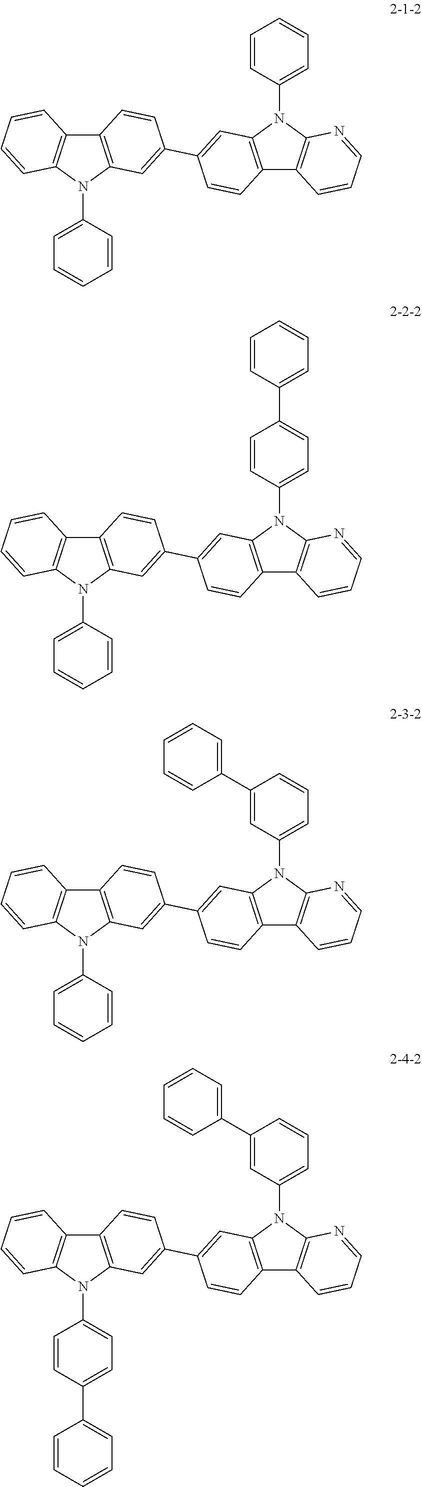 [SMILES: c1ccc(-n2c3ccccc3c3ccc(-c4ccc5c6cccnc6n(-c6ccccc6)c5c4)cc32)cc1 ; c1ccc(-c2ccc(-n3c4cc(-c5ccc6c7ccccc7n(-c7ccccc7)c6c5)ccc4c4cccnc43)cc2)cc1 ; c1ccc(-c2cccc(-n3c4cc(-c5ccc6c7ccccc7n(-c7ccccc7)c6c5)ccc4c4cccnc43)c2)cc1 ; c1ccc(-c2ccc(-n3c4ccccc4c4ccc(-c5ccc6c7cccnc7n(-c7cccc(-c8ccccc8)c7)c6c5)cc43)cc2)cc1]

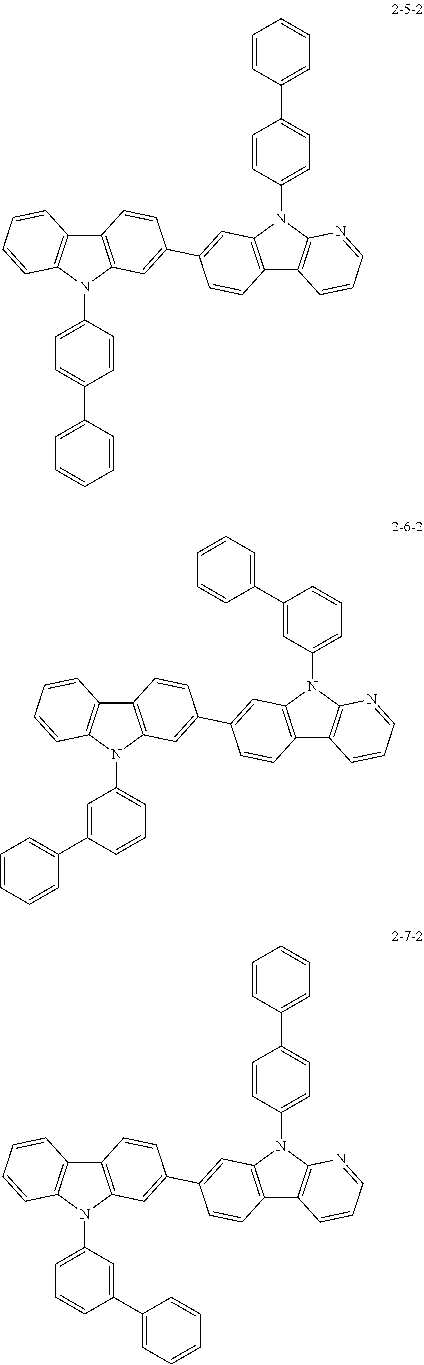 [SMILES: c1ccc(-c2ccc(-n3c4ccccc4c4ccc(-c5ccc6c7cccnc7n(-c7ccc(-c8ccccc8)cc7)c6c5)cc43)cc2)cc1 ; c1ccc(-c2cccc(-n3c4ccccc4c4ccc(-c5ccc6c7cccnc7n(-c7cccc(-c8ccccc8)c7)c6c5)cc43)c2)cc1 ; c1ccc(-c2ccc(-n3c4cc(-c5ccc6c7ccccc7n(-c7cccc(-c8ccccc8)c7)c6c5)ccc4c4cccnc43)cc2)cc1]

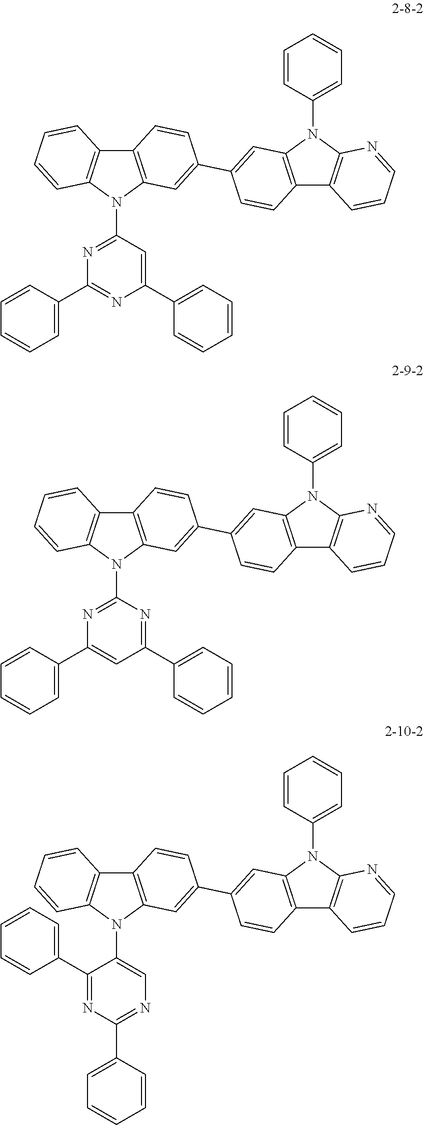 [SMILES: c1ccc(-c2cc(-n3c4ccccc4c4ccc(-c5ccc6c7cccnc7n(-c7ccccc7)c6c5)cc43)nc(-c3ccccc3)n2)cc1 ; c1ccc(-c2cc(-c3ccccc3)nc(-n3c4ccccc4c4ccc(-c5ccc6c7cccnc7n(-c7ccccc7)c6c5)cc43)n2)cc1 ; c1ccc(-c2ncc(-n3c4ccccc4c4ccc(-c5ccc6c7cccnc7n(-c7ccccc7)c6c5)cc43)c(-c3ccccc3)n2)cc1]

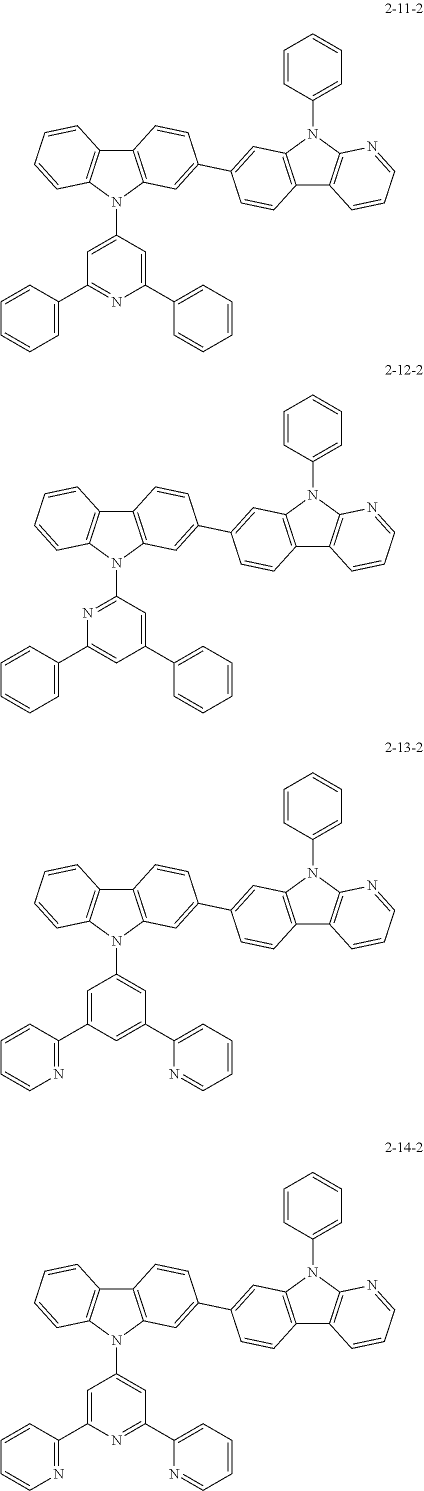 [SMILES: c1ccc(-c2cc(-n3c4ccccc4c4ccc(-c5ccc6c7cccnc7n(-c7ccccc7)c6c5)cc43)cc(-c3ccccc3)n2)cc1 ; c1ccc(-c2cc(-c3ccccc3)nc(-n3c4ccccc4c4ccc(-c5ccc6c7cccnc7n(-c7ccccc7)c6c5)cc43)c2)cc1 ; c1ccc(-n2c3cc(-c4ccc5c6ccccc6n(-c6cc(-c7ccccn7)cc(-c7ccccn7)c6)c5c4)ccc3c3cccnc32)cc1 ; c1ccc(-n2c3cc(-c4ccc5c6ccccc6n(-c6cc(-c7ccccn7)nc(-c7ccccn7)c6)c5c4)ccc3c3cccnc32)cc1]

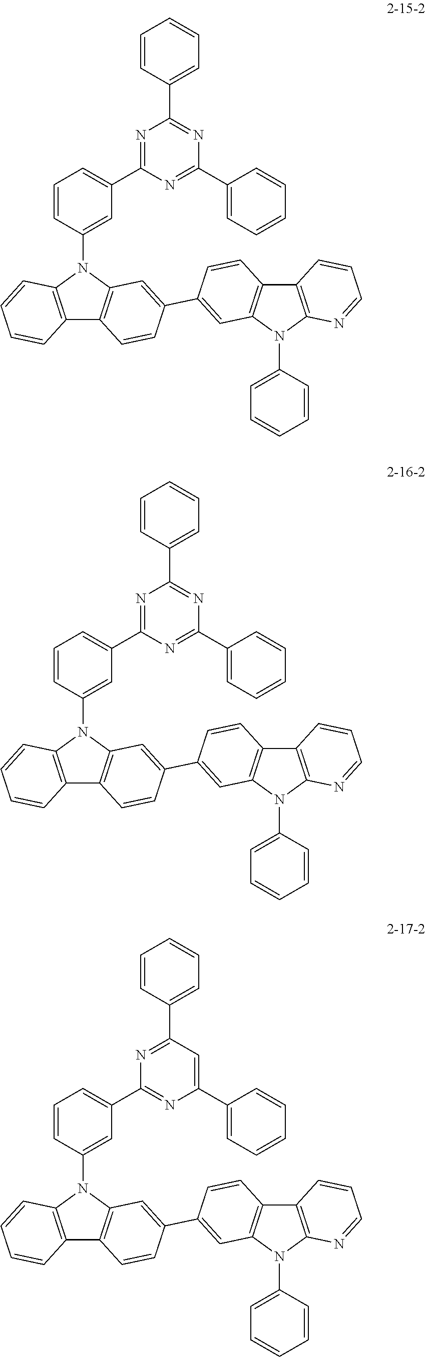 [SMILES: c1ccc(-c2nc(-c3ccccc3)nc(-c3cccc(-n4c5ccccc5c5ccc(-c6ccc7c8cccnc8n(-c8ccccc8)c7c6)cc54)c3)n2)cc1 ; c1ccc(-c2nc(-c3ccccc3)nc(-c3cccc(-n4c5ccccc5c5ccc(-c6ccc7c8cccnc8n(-c8ccccc8)c7c6)cc54)c3)n2)cc1 ; c1ccc(-c2cc(-c3ccccc3)nc(-c3cccc(-n4c5ccccc5c5ccc(-c6ccc7c8cccnc8n(-c8ccccc8)c7c6)cc54)c3)n2)cc1]

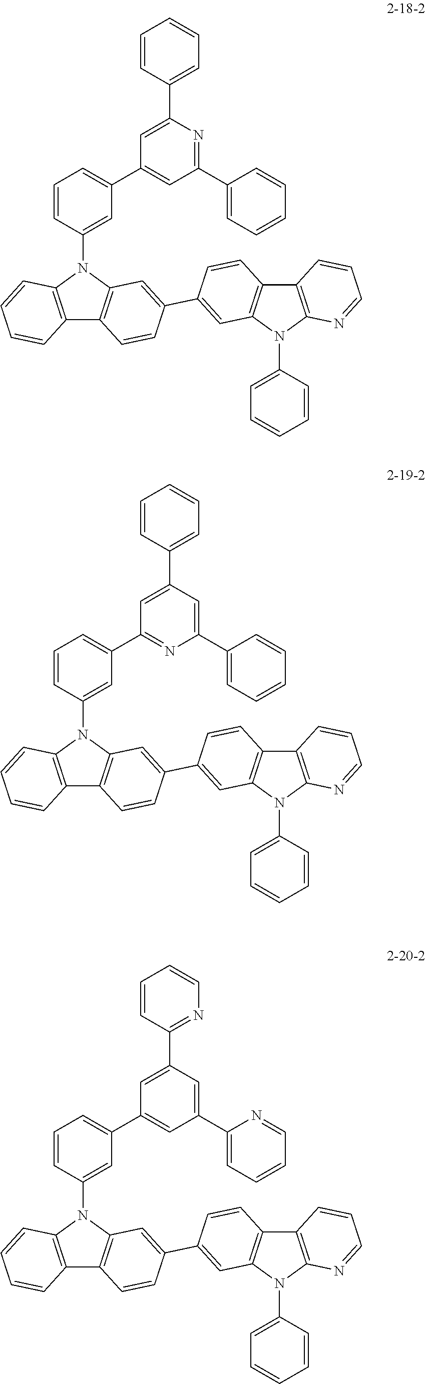 [SMILES: c1ccc(-c2cc(-c3cccc(-n4c5ccccc5c5ccc(-c6ccc7c8cccnc8n(-c8ccccc8)c7c6)cc54)c3)cc(-c3ccccc3)n2)cc1 ; c1ccc(-c2cc(-c3ccccc3)nc(-c3cccc(-n4c5ccccc5c5ccc(-c6ccc7c8cccnc8n(-c8ccccc8)c7c6)cc54)c3)c2)cc1 ; c1ccc(-n2c3cc(-c4ccc5c6ccccc6n(-c6cccc(-c7cc(-c8ccccn8)cc(-c8ccccn8)c7)c6)c5c4)ccc3c3cccnc32)cc1]

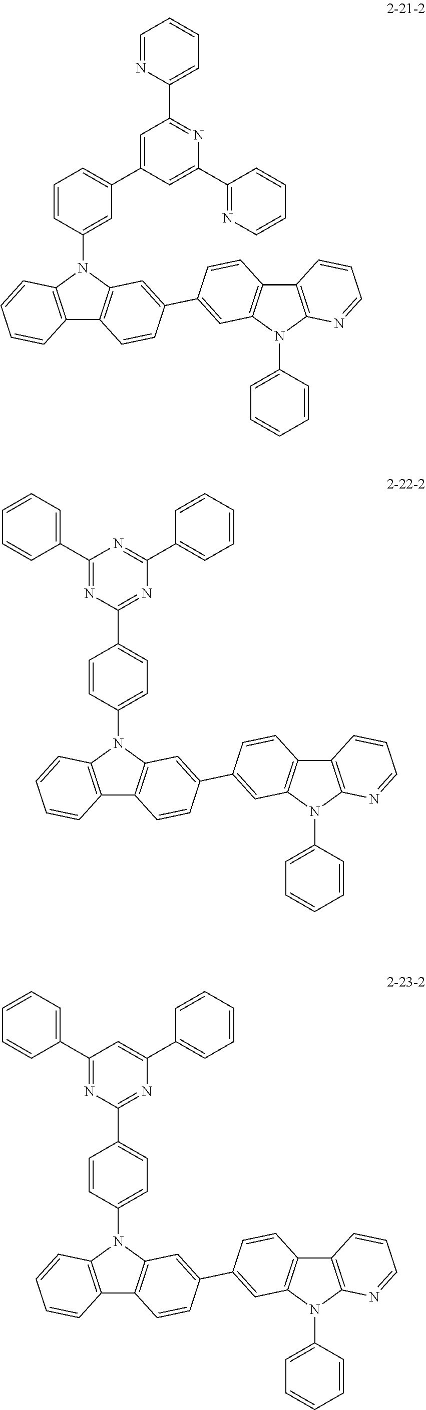 [SMILES: c1ccc(-n2c3cc(-c4ccc5c6ccccc6n(-c6cccc(-c7cc(-c8ccccn8)nc(-c8ccccn8)c7)c6)c5c4)ccc3c3cccnc32)cc1 ; c1ccc(-c2nc(-c3ccccc3)nc(-c3ccc(-n4c5ccccc5c5ccc(-c6ccc7c8cccnc8n(-c8ccccc8)c7c6)cc54)cc3)n2)cc1 ; c1ccc(-c2cc(-c3ccccc3)nc(-c3ccc(-n4c5ccccc5c5ccc(-c6ccc7c8cccnc8n(-c8ccccc8)c7c6)cc54)cc3)n2)cc1]

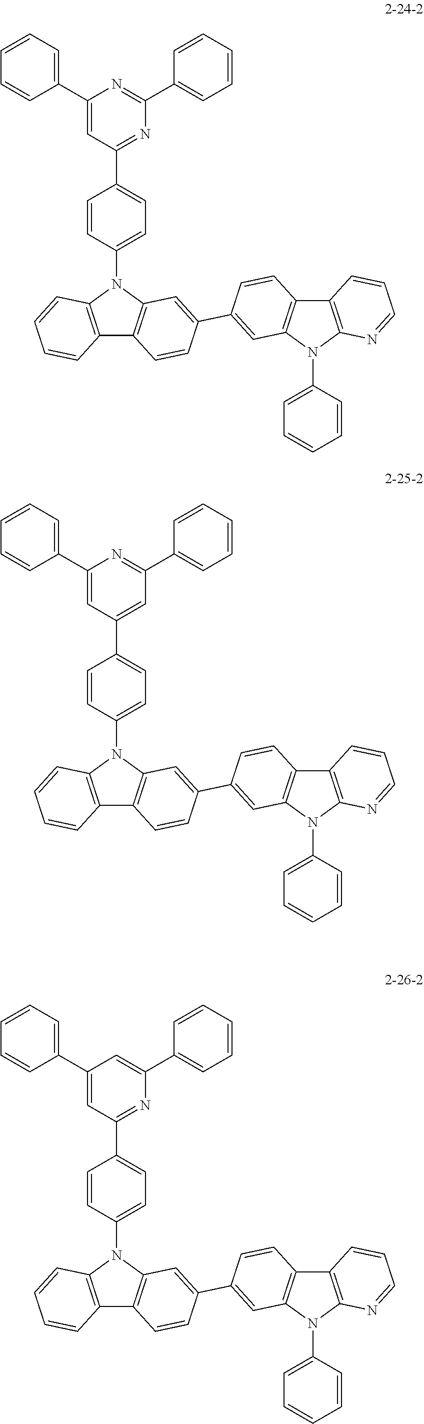 [SMILES: c1ccc(-c2cc(-c3ccc(-n4c5ccccc5c5ccc(-c6ccc7c8cccnc8n(-c8ccccc8)c7c6)cc54)cc3)nc(-c3ccccc3)n2)cc1 ; c1ccc(-c2cc(-c3ccc(-n4c5ccccc5c5ccc(-c6ccc7c8cccnc8n(-c8ccccc8)c7c6)cc54)cc3)cc(-c3ccccc3)n2)cc1 ; c1ccc(-c2cc(-c3ccccc3)nc(-c3ccc(-n4c5ccccc5c5ccc(-c6ccc7c8cccnc8n(-c8ccccc8)c7c6)cc54)cc3)c2)cc1]

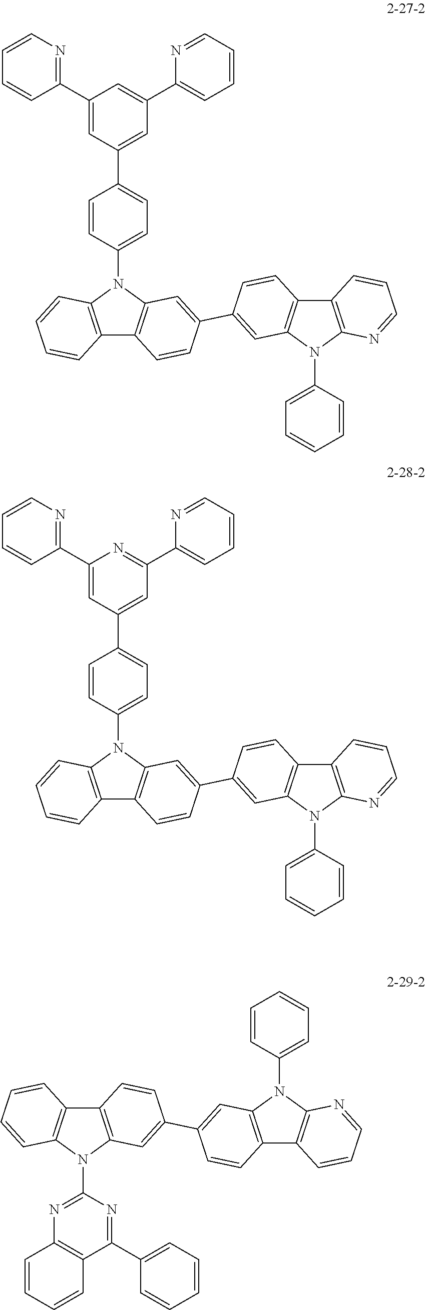 [SMILES: c1ccc(-n2c3cc(-c4ccc5c6ccccc6n(-c6ccc(-c7cc(-c8ccccn8)cc(-c8ccccn8)c7)cc6)c5c4)ccc3c3cccnc32)cc1 ; c1ccc(-n2c3cc(-c4ccc5c6ccccc6n(-c6ccc(-c7cc(-c8ccccn8)nc(-c8ccccn8)c7)cc6)c5c4)ccc3c3cccnc32)cc1 ; c1ccc(-c2nc(-n3c4ccccc4c4ccc(-c5ccc6c7cccnc7n(-c7ccccc7)c6c5)cc43)nc3ccccc23)cc1]

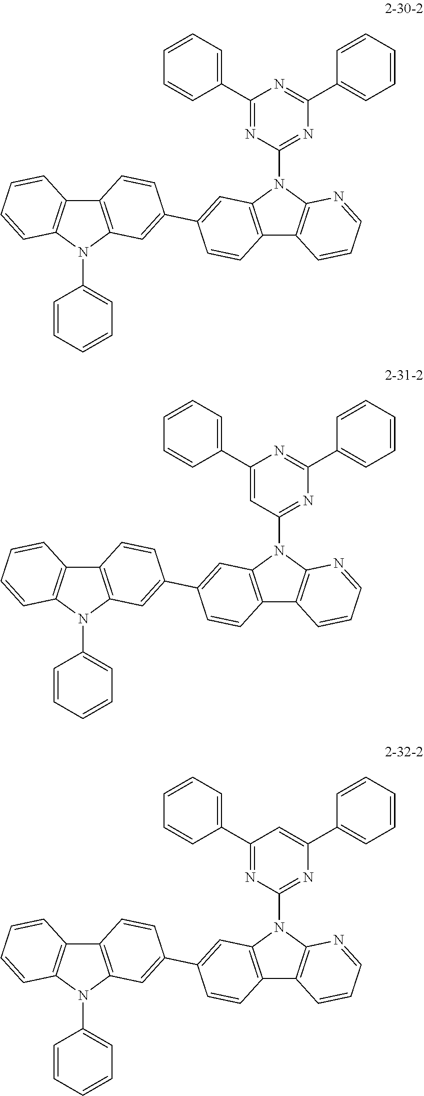 [SMILES: c1ccc(-c2nc(-c3ccccc3)nc(-n3c4cc(-c5ccc6c7ccccc7n(-c7ccccc7)c6c5)ccc4c4cccnc43)n2)cc1 ; c1ccc(-c2cc(-n3c4cc(-c5ccc6c7ccccc7n(-c7ccccc7)c6c5)ccc4c4cccnc43)nc(-c3ccccc3)n2)cc1 ; c1ccc(-c2cc(-c3ccccc3)nc(-n3c4cc(-c5ccc6c7ccccc7n(-c7ccccc7)c6c5)ccc4c4cccnc43)n2)cc1]

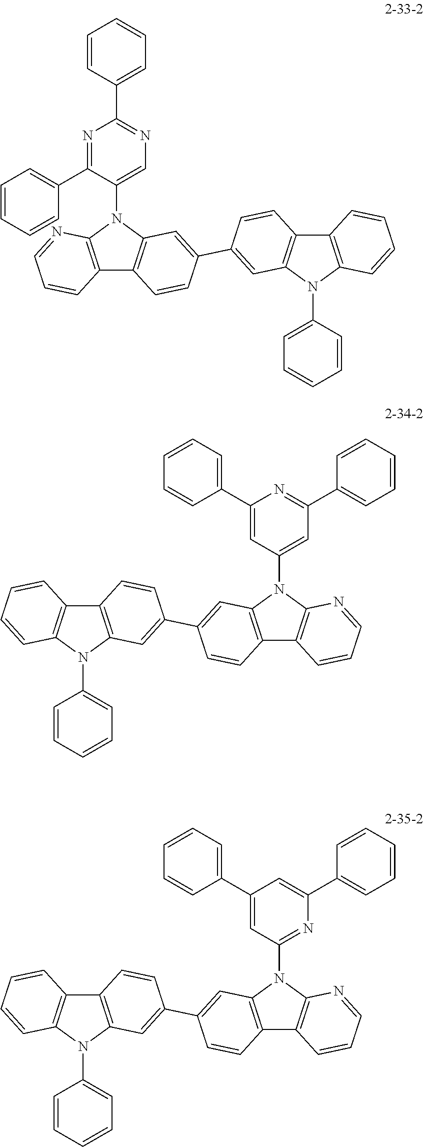 [SMILES: c1ccc(-c2ncc(-n3c4cc(-c5ccc6c7ccccc7n(-c7ccccc7)c6c5)ccc4c4cccnc43)c(-c3ccccc3)n2)cc1 ; c1ccc(-c2cc(-n3c4cc(-c5ccc6c7ccccc7n(-c7ccccc7)c6c5)ccc4c4cccnc43)cc(-c3ccccc3)n2)cc1 ; c1ccc(-c2cc(-c3ccccc3)nc(-n3c4cc(-c5ccc6c7ccccc7n(-c7ccccc7)c6c5)ccc4c4cccnc43)c2)cc1]

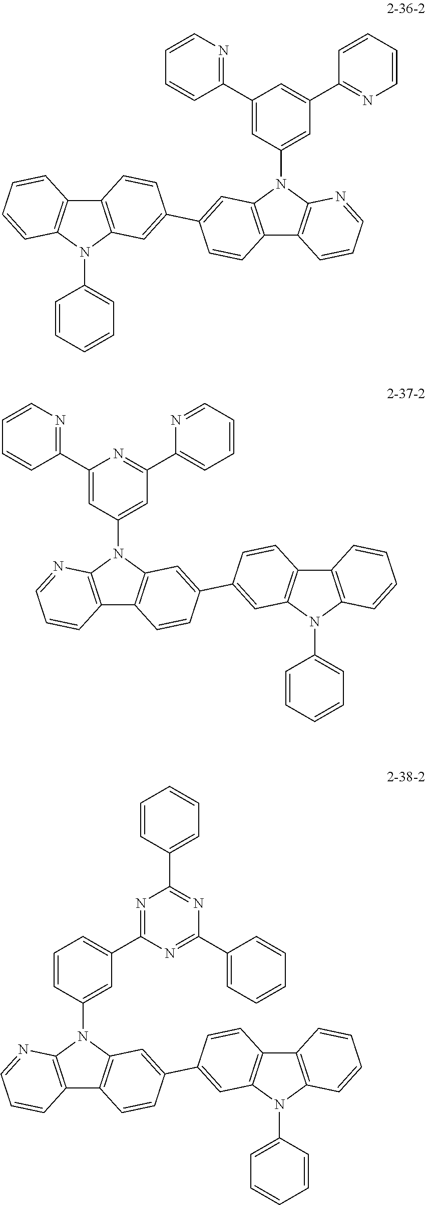 [SMILES: c1ccc(-n2c3ccccc3c3ccc(-c4ccc5c6cccnc6n(-c6cc(-c7ccccn7)cc(-c7ccccn7)c6)c5c4)cc32)cc1 ; c1ccc(-n2c3ccccc3c3ccc(-c4ccc5c6cccnc6n(-c6cc(-c7ccccn7)nc(-c7ccccn7)c6)c5c4)cc32)cc1 ; c1ccc(-c2nc(-c3ccccc3)nc(-c3cccc(-n4c5cc(-c6ccc7c8ccccc8n(-c8ccccc8)c7c6)ccc5c5cccnc54)c3)n2)cc1]

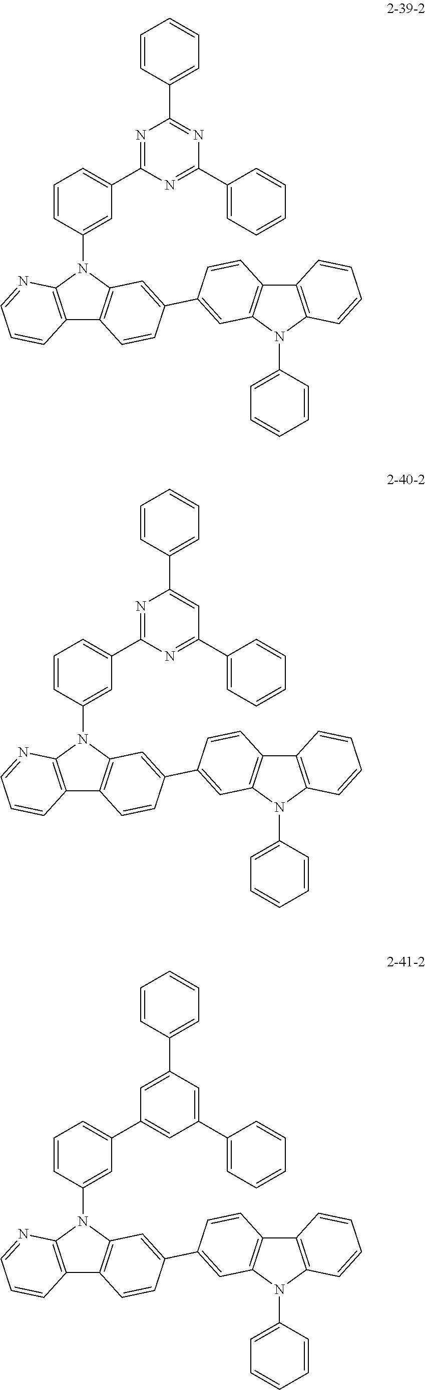 [SMILES: c1ccc(-c2nc(-c3ccccc3)nc(-c3cccc(-n4c5cc(-c6ccc7c8ccccc8n(-c8ccccc8)c7c6)ccc5c5cccnc54)c3)n2)cc1 ; c1ccc(-c2cc(-c3ccccc3)nc(-c3cccc(-n4c5cc(-c6ccc7c8ccccc8n(-c8ccccc8)c7c6)ccc5c5cccnc54)c3)n2)cc1 ; c1ccc(-c2cc(-c3ccccc3)cc(-c3cccc(-n4c5cc(-c6ccc7c8ccccc8n(-c8ccccc8)c7c6)ccc5c5cccnc54)c3)c2)cc1]

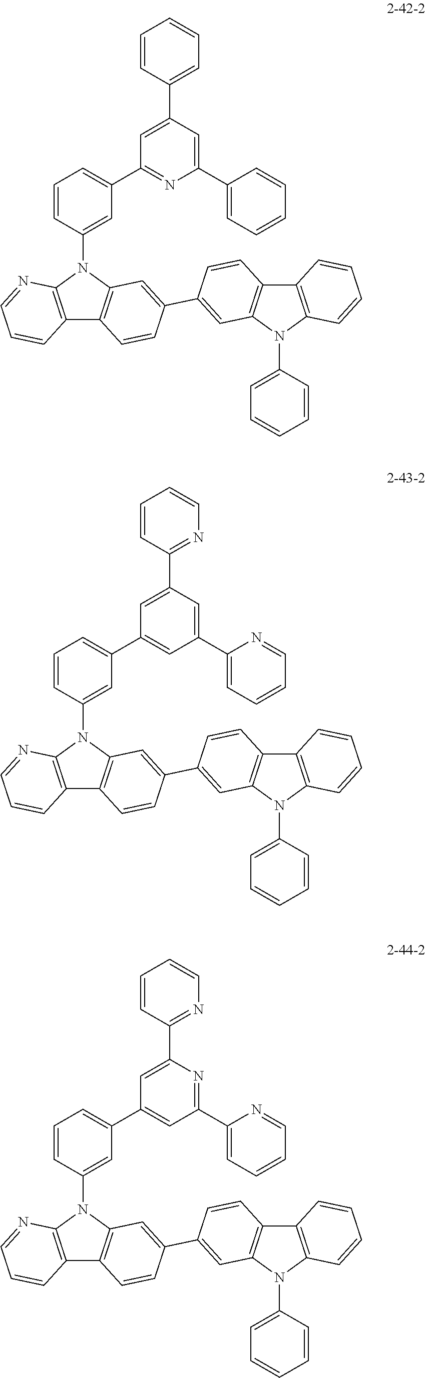 [SMILES: c1ccc(-c2cc(-c3ccccc3)nc(-c3cccc(-n4c5cc(-c6ccc7c8ccccc8n(-c8ccccc8)c7c6)ccc5c5cccnc54)c3)c2)cc1 ; c1ccc(-n2c3ccccc3c3ccc(-c4ccc5c6cccnc6n(-c6cccc(-c7cc(-c8ccccn8)cc(-c8ccccn8)c7)c6)c5c4)cc32)cc1 ; c1ccc(-n2c3ccccc3c3ccc(-c4ccc5c6cccnc6n(-c6cccc(-c7cc(-c8ccccn8)nc(-c8ccccn8)c7)c6)c5c4)cc32)cc1]

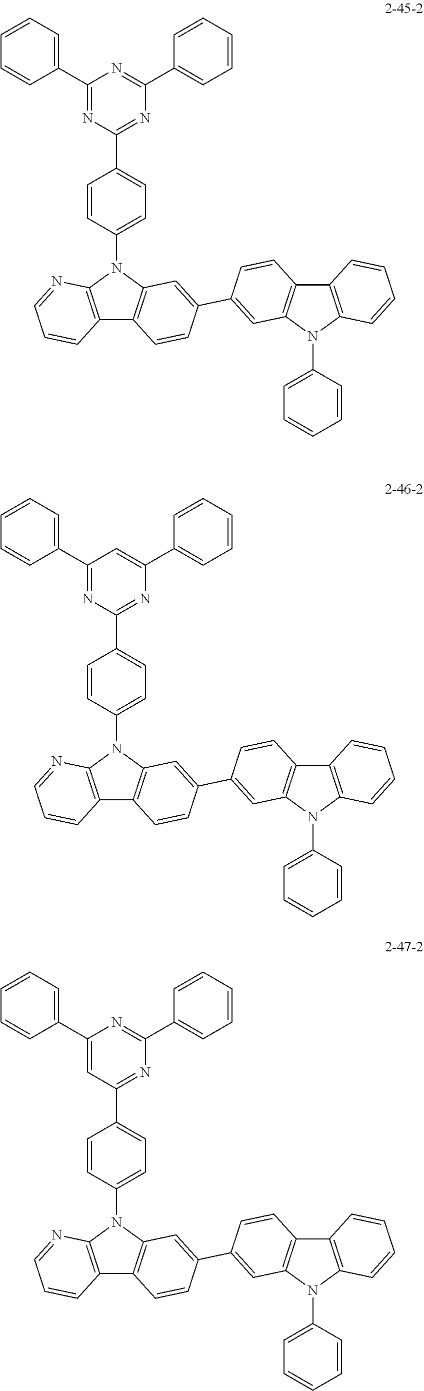 [SMILES: c1ccc(-c2nc(-c3ccccc3)nc(-c3ccc(-n4c5cc(-c6ccc7c8ccccc8n(-c8ccccc8)c7c6)ccc5c5cccnc54)cc3)n2)cc1 ; c1ccc(-c2cc(-c3ccccc3)nc(-c3ccc(-n4c5cc(-c6ccc7c8ccccc8n(-c8ccccc8)c7c6)ccc5c5cccnc54)cc3)n2)cc1 ; c1ccc(-c2cc(-c3ccc(-n4c5cc(-c6ccc7c8ccccc8n(-c8ccccc8)c7c6)ccc5c5cccnc54)cc3)nc(-c3ccccc3)n2)cc1]

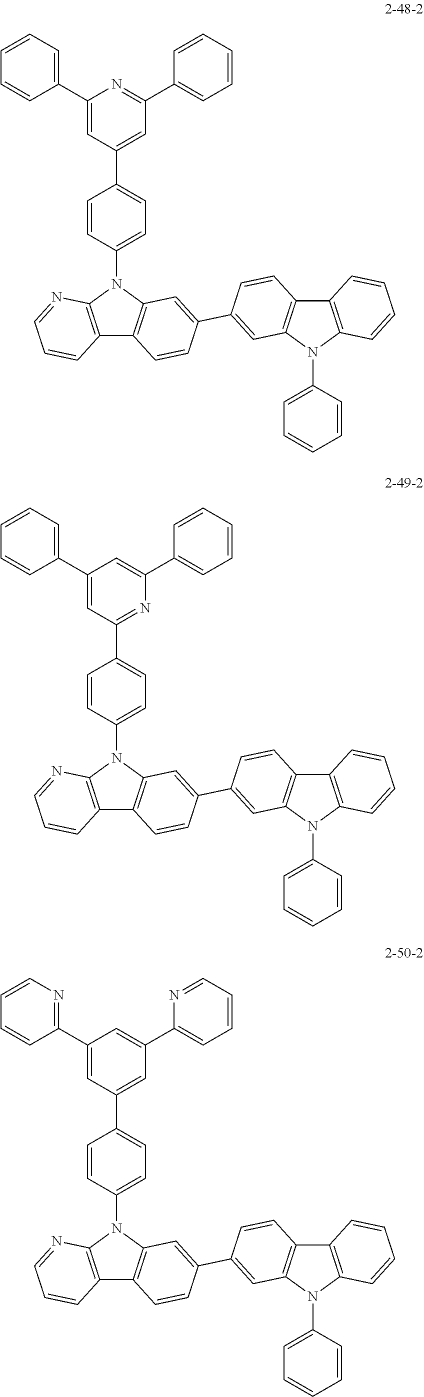 [SMILES: c1ccc(-c2cc(-c3ccc(-n4c5cc(-c6ccc7c8ccccc8n(-c8ccccc8)c7c6)ccc5c5cccnc54)cc3)cc(-c3ccccc3)n2)cc1 ; c1ccc(-c2cc(-c3ccccc3)nc(-c3ccc(-n4c5cc(-c6ccc7c8ccccc8n(-c8ccccc8)c7c6)ccc5c5cccnc54)cc3)c2)cc1 ; c1ccc(-n2c3ccccc3c3ccc(-c4ccc5c6cccnc6n(-c6ccc(-c7cc(-c8ccccn8)cc(-c8ccccn8)c7)cc6)c5c4)cc32)cc1]

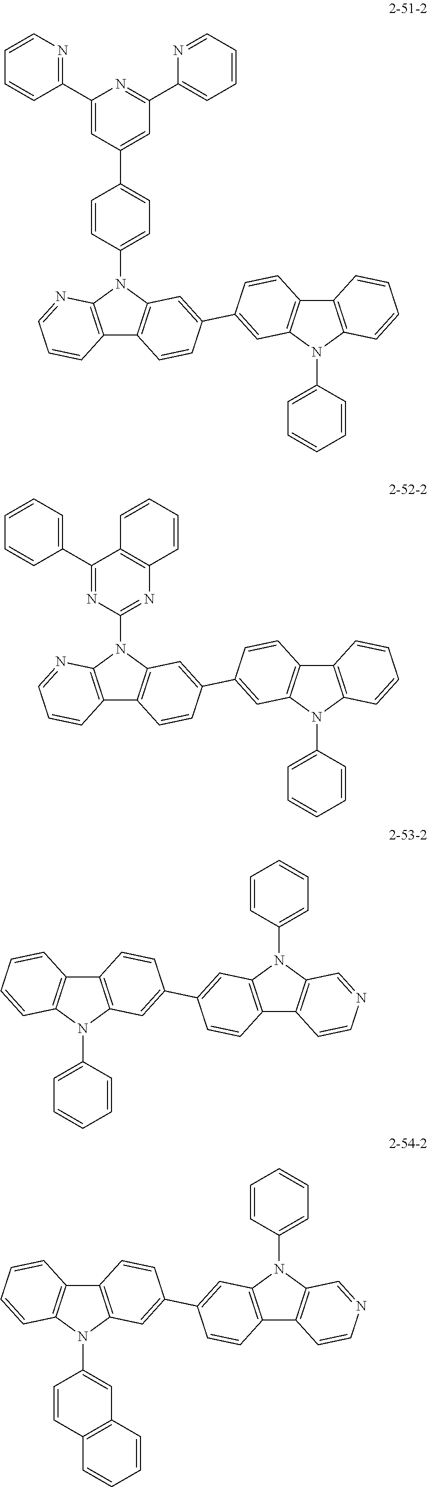 [SMILES: c1ccc(-n2c3ccccc3c3ccc(-c4ccc5c6cccnc6n(-c6ccc(-c7cc(-c8ccccn8)nc(-c8ccccn8)c7)cc6)c5c4)cc32)cc1 ; c1ccc(-c2nc(-n3c4cc(-c5ccc6c7ccccc7n(-c7ccccc7)c6c5)ccc4c4cccnc43)nc3ccccc23)cc1 ; c1ccc(-n2c3ccccc3c3ccc(-c4ccc5c6ccncc6n(-c6ccccc6)c5c4)cc32)cc1 ; c1ccc(-n2c3cnccc3c3ccc(-c4ccc5c6ccccc6n(-c6ccc7ccccc7c6)c5c4)cc32)cc1]

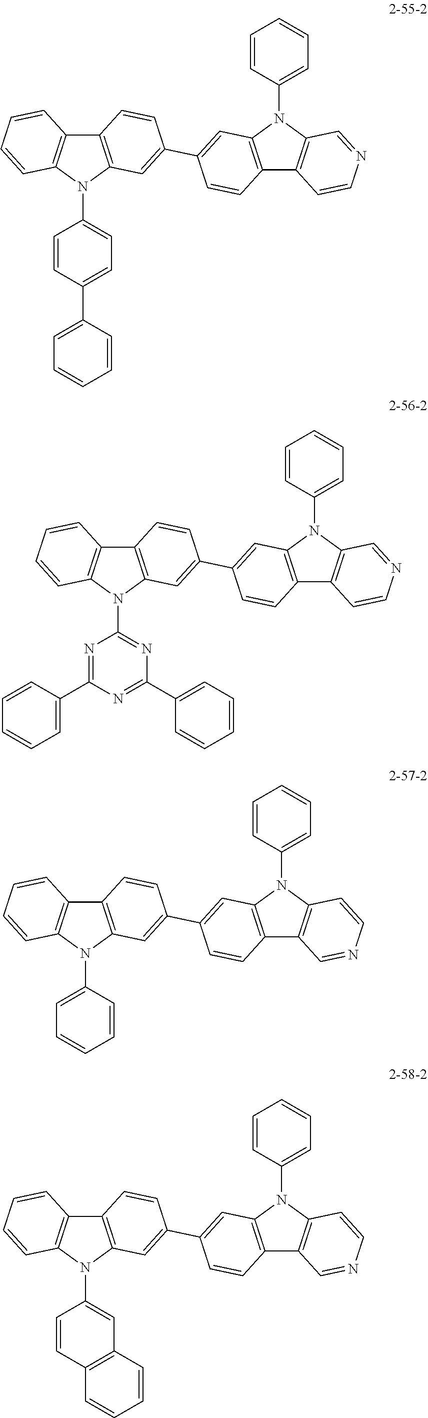 [SMILES: c1ccc(-c2ccc(-n3c4ccccc4c4ccc(-c5ccc6c7ccncc7n(-c7ccccc7)c6c5)cc43)cc2)cc1 ; c1ccc(-c2nc(-c3ccccc3)nc(-n3c4ccccc4c4ccc(-c5ccc6c7ccncc7n(-c7ccccc7)c6c5)cc43)n2)cc1 ; c1ccc(-n2c3ccccc3c3ccc(-c4ccc5c6cnccc6n(-c6ccccc6)c5c4)cc32)cc1 ; c1ccc(-n2c3ccncc3c3ccc(-c4ccc5c6ccccc6n(-c6ccc7ccccc7c6)c5c4)cc32)cc1]

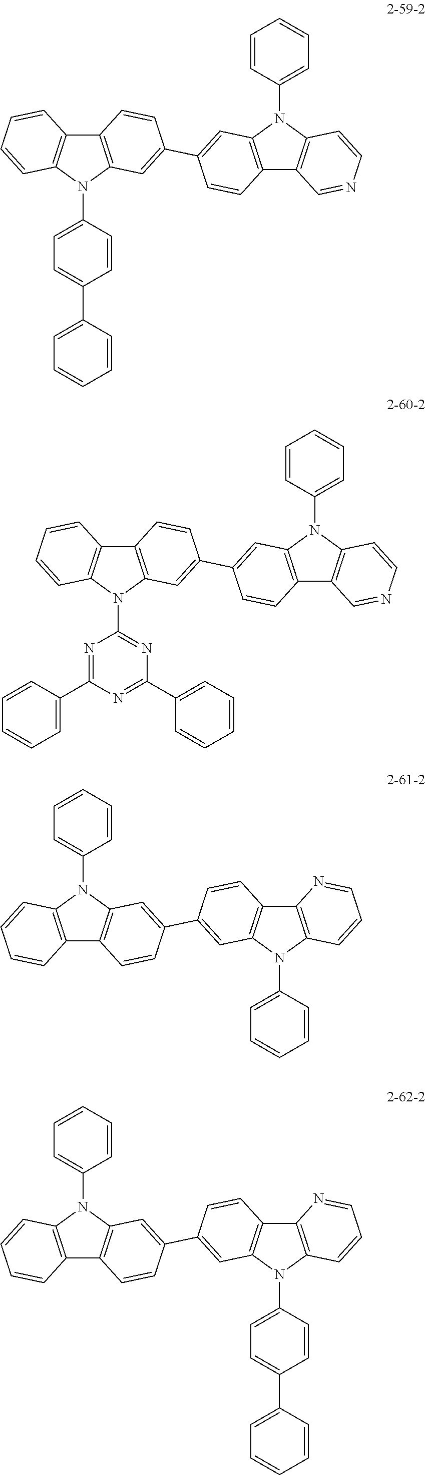 [SMILES: c1ccc(-c2ccc(-n3c4ccccc4c4ccc(-c5ccc6c7cnccc7n(-c7ccccc7)c6c5)cc43)cc2)cc1 ; c1ccc(-c2nc(-c3ccccc3)nc(-n3c4ccccc4c4ccc(-c5ccc6c7cnccc7n(-c7ccccc7)c6c5)cc43)n2)cc1 ; c1ccc(-n2c3ccccc3c3ccc(-c4ccc5c6ncccc6n(-c6ccccc6)c5c4)cc32)cc1 ; c1ccc(-c2ccc(-n3c4cc(-c5ccc6c7ccccc7n(-c7ccccc7)c6c5)ccc4c4ncccc43)cc2)cc1]

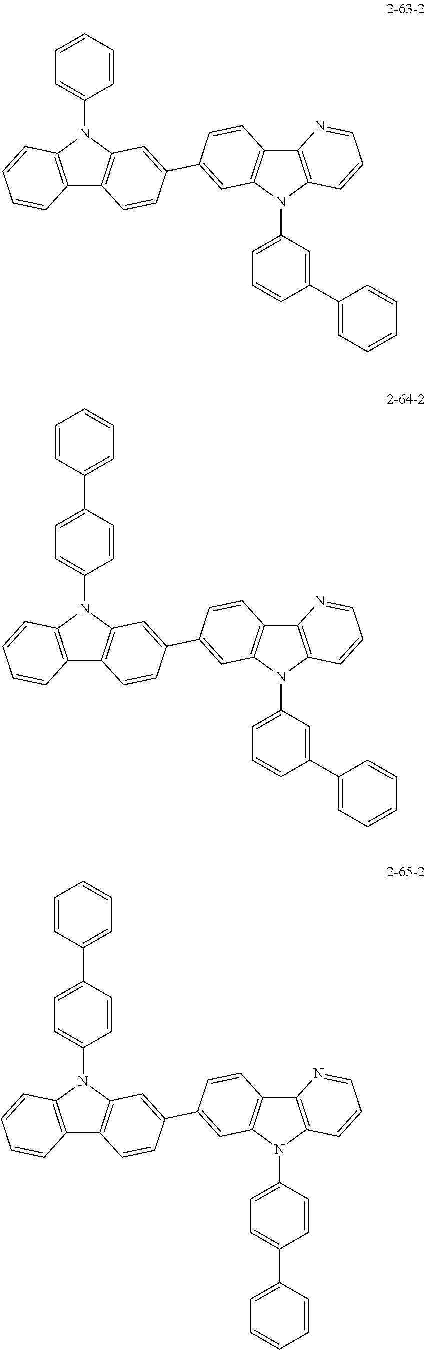 [SMILES: c1ccc(-c2cccc(-n3c4cc(-c5ccc6c7ccccc7n(-c7ccccc7)c6c5)ccc4c4ncccc43)c2)cc1 ; c1ccc(-c2ccc(-n3c4ccccc4c4ccc(-c5ccc6c7ncccc7n(-c7cccc(-c8ccccc8)c7)c6c5)cc43)cc2)cc1 ; c1ccc(-c2ccc(-n3c4ccccc4c4ccc(-c5ccc6c7ncccc7n(-c7ccc(-c8ccccc8)cc7)c6c5)cc43)cc2)cc1]

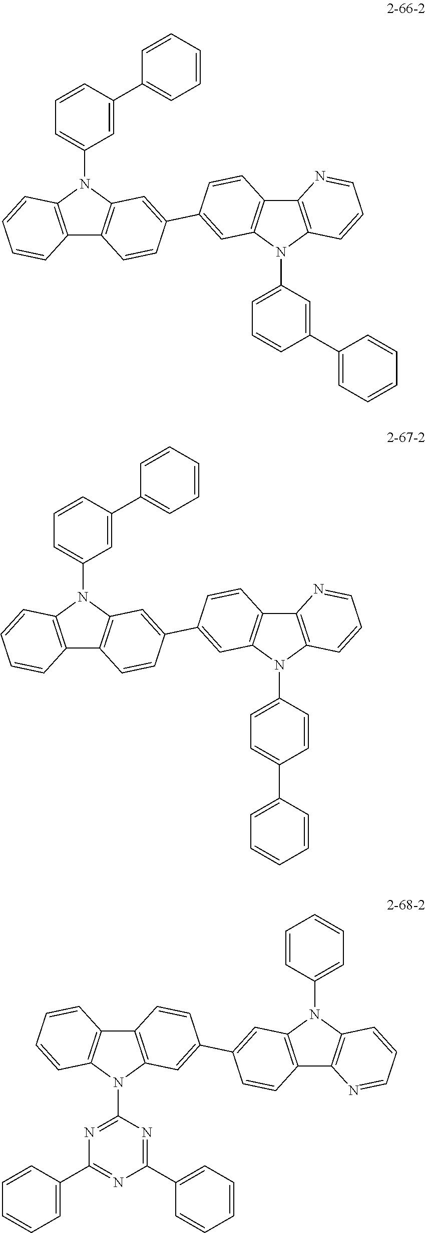 [SMILES: c1ccc(-c2cccc(-n3c4ccccc4c4ccc(-c5ccc6c7ncccc7n(-c7cccc(-c8ccccc8)c7)c6c5)cc43)c2)cc1 ; c1ccc(-c2ccc(-n3c4cc(-c5ccc6c7ccccc7n(-c7cccc(-c8ccccc8)c7)c6c5)ccc4c4ncccc43)cc2)cc1 ; c1ccc(-c2nc(-c3ccccc3)nc(-n3c4ccccc4c4ccc(-c5ccc6c7ncccc7n(-c7ccccc7)c6c5)cc43)n2)cc1]

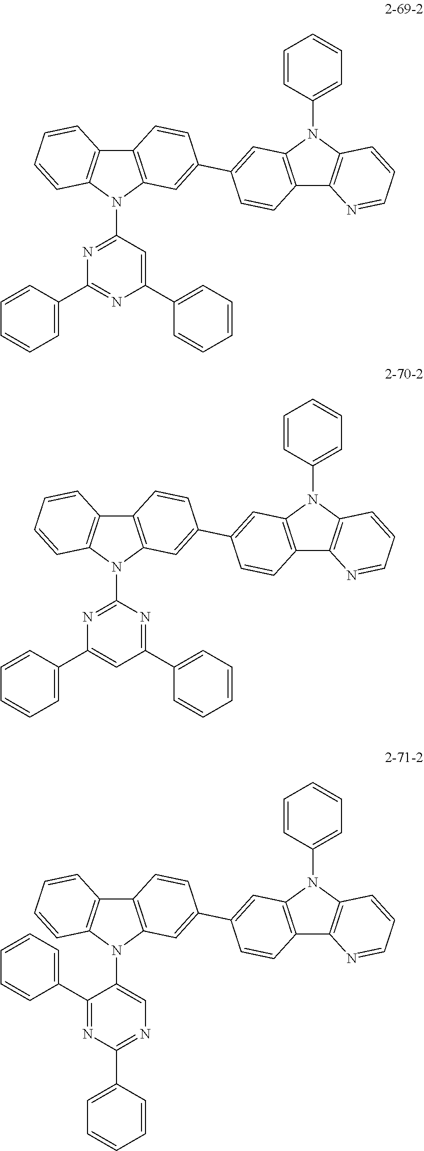 [SMILES: c1ccc(-c2cc(-n3c4ccccc4c4ccc(-c5ccc6c7ncccc7n(-c7ccccc7)c6c5)cc43)nc(-c3ccccc3)n2)cc1 ; c1ccc(-c2cc(-c3ccccc3)nc(-n3c4ccccc4c4ccc(-c5ccc6c7ncccc7n(-c7ccccc7)c6c5)cc43)n2)cc1 ; c1ccc(-c2ncc(-n3c4ccccc4c4ccc(-c5ccc6c7ncccc7n(-c7ccccc7)c6c5)cc43)c(-c3ccccc3)n2)cc1]

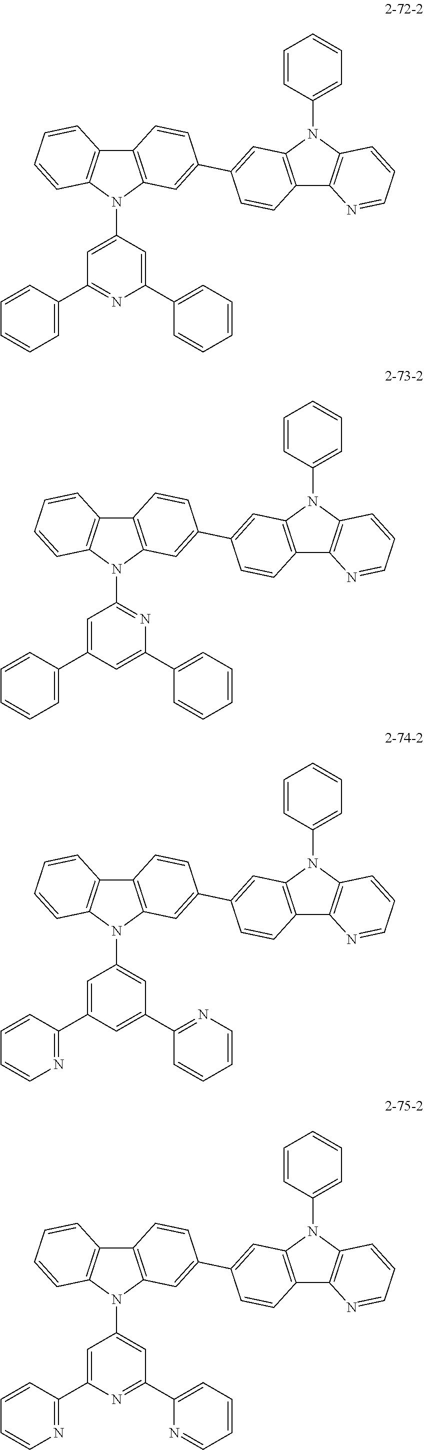 [SMILES: c1ccc(-c2cc(-n3c4ccccc4c4ccc(-c5ccc6c7ncccc7n(-c7ccccc7)c6c5)cc43)cc(-c3ccccc3)n2)cc1 ; c1ccc(-c2cc(-c3ccccc3)nc(-n3c4ccccc4c4ccc(-c5ccc6c7ncccc7n(-c7ccccc7)c6c5)cc43)c2)cc1 ; c1ccc(-n2c3cc(-c4ccc5c6ccccc6n(-c6cc(-c7ccccn7)cc(-c7ccccn7)c6)c5c4)ccc3c3ncccc32)cc1 ; c1ccc(-n2c3cc(-c4ccc5c6ccccc6n(-c6cc(-c7ccccn7)nc(-c7ccccn7)c6)c5c4)ccc3c3ncccc32)cc1]

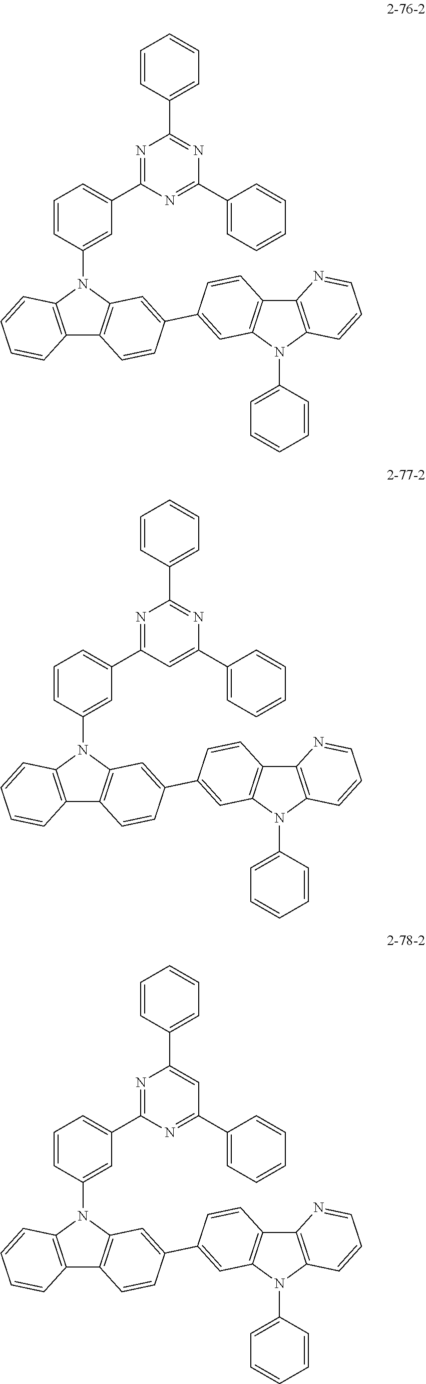 [SMILES: c1ccc(-c2nc(-c3ccccc3)nc(-c3cccc(-n4c5ccccc5c5ccc(-c6ccc7c8ncccc8n(-c8ccccc8)c7c6)cc54)c3)n2)cc1 ; c1ccc(-c2cc(-c3cccc(-n4c5ccccc5c5ccc(-c6ccc7c8ncccc8n(-c8ccccc8)c7c6)cc54)c3)nc(-c3ccccc3)n2)cc1 ; c1ccc(-c2cc(-c3ccccc3)nc(-c3cccc(-n4c5ccccc5c5ccc(-c6ccc7c8ncccc8n(-c8ccccc8)c7c6)cc54)c3)n2)cc1]

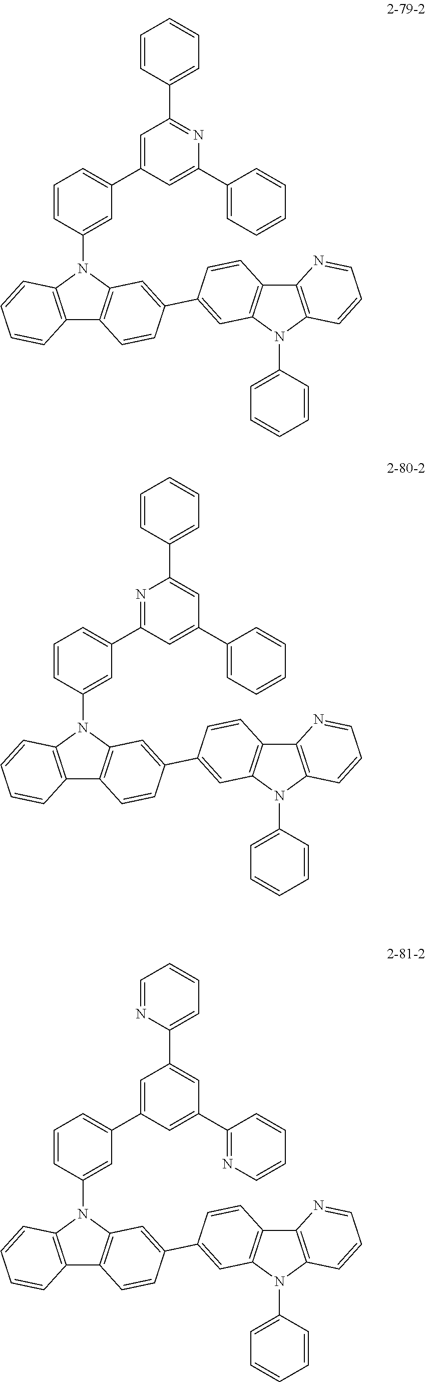 [SMILES: c1ccc(-c2cc(-c3cccc(-n4c5ccccc5c5ccc(-c6ccc7c8ncccc8n(-c8ccccc8)c7c6)cc54)c3)cc(-c3ccccc3)n2)cc1 ; c1ccc(-c2cc(-c3ccccc3)nc(-c3cccc(-n4c5ccccc5c5ccc(-c6ccc7c8ncccc8n(-c8ccccc8)c7c6)cc54)c3)c2)cc1 ; c1ccc(-n2c3cc(-c4ccc5c6ccccc6n(-c6cccc(-c7cc(-c8ccccn8)cc(-c8ccccn8)c7)c6)c5c4)ccc3c3ncccc32)cc1]

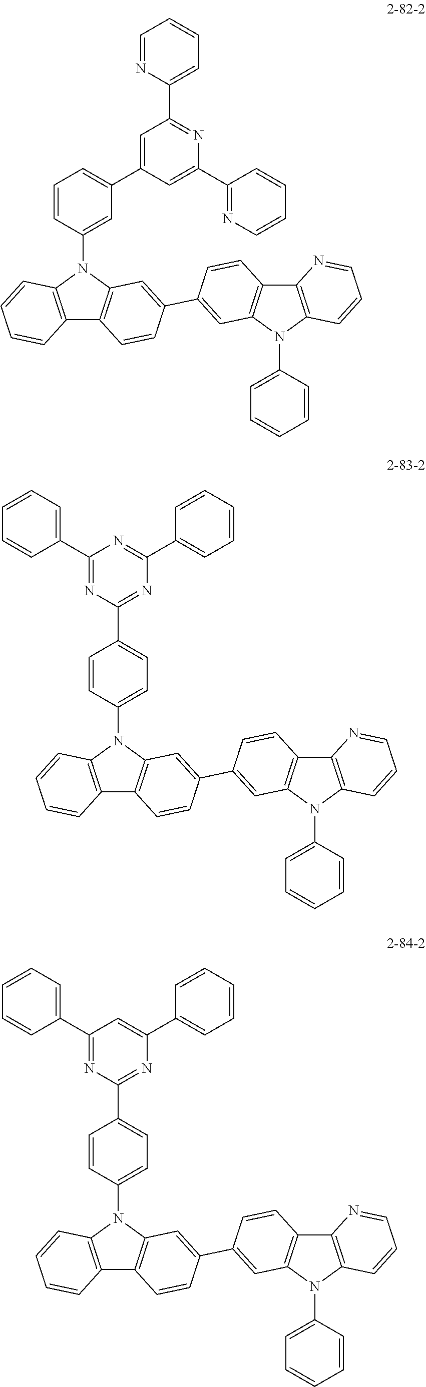 [SMILES: c1ccc(-n2c3cc(-c4ccc5c6ccccc6n(-c6cccc(-c7cc(-c8ccccn8)nc(-c8ccccn8)c7)c6)c5c4)ccc3c3ncccc32)cc1 ; c1ccc(-c2nc(-c3ccccc3)nc(-c3ccc(-n4c5ccccc5c5ccc(-c6ccc7c8ncccc8n(-c8ccccc8)c7c6)cc54)cc3)n2)cc1 ; c1ccc(-c2cc(-c3ccccc3)nc(-c3ccc(-n4c5ccccc5c5ccc(-c6ccc7c8ncccc8n(-c8ccccc8)c7c6)cc54)cc3)n2)cc1]

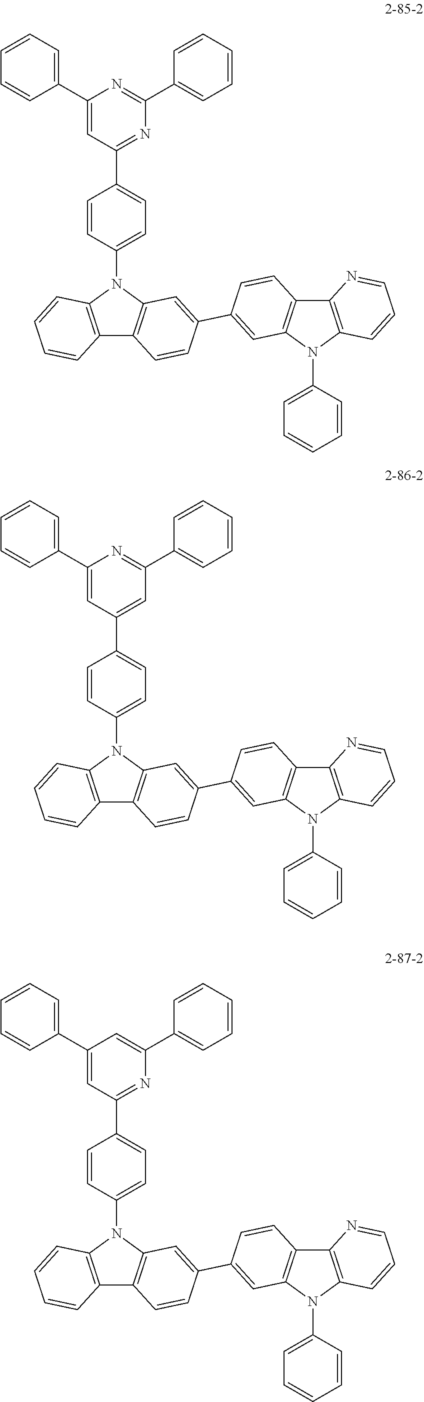 [SMILES: c1ccc(-c2cc(-c3ccc(-n4c5ccccc5c5ccc(-c6ccc7c8ncccc8n(-c8ccccc8)c7c6)cc54)cc3)nc(-c3ccccc3)n2)cc1 ; c1ccc(-c2cc(-c3ccc(-n4c5ccccc5c5ccc(-c6ccc7c8ncccc8n(-c8ccccc8)c7c6)cc54)cc3)cc(-c3ccccc3)n2)cc1 ; c1ccc(-c2cc(-c3ccccc3)nc(-c3ccc(-n4c5ccccc5c5ccc(-c6ccc7c8ncccc8n(-c8ccccc8)c7c6)cc54)cc3)c2)cc1]

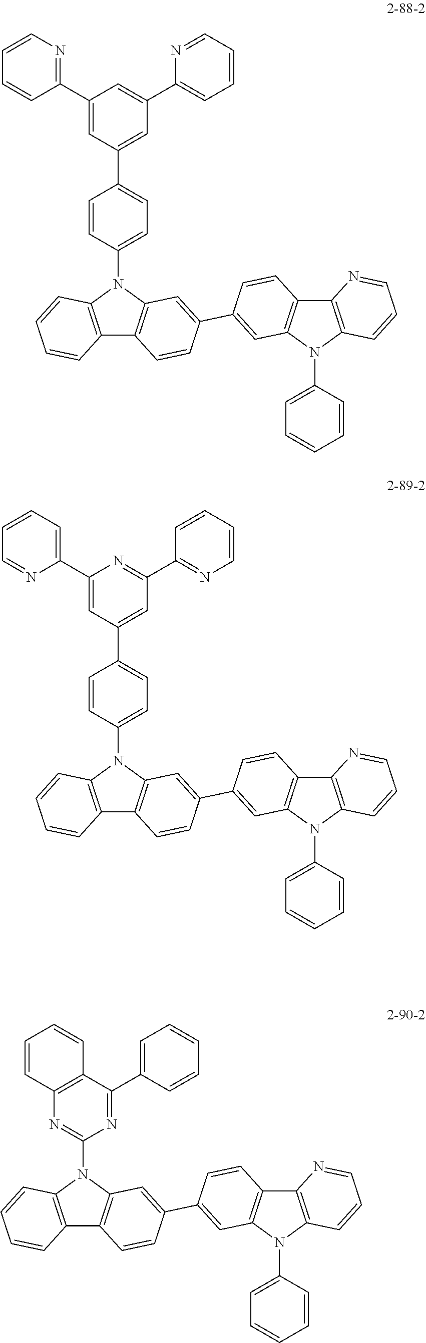 [SMILES: c1ccc(-n2c3cc(-c4ccc5c6ccccc6n(-c6ccc(-c7cc(-c8ccccn8)cc(-c8ccccn8)c7)cc6)c5c4)ccc3c3ncccc32)cc1 ; c1ccc(-n2c3cc(-c4ccc5c6ccccc6n(-c6ccc(-c7cc(-c8ccccn8)nc(-c8ccccn8)c7)cc6)c5c4)ccc3c3ncccc32)cc1 ; c1ccc(-c2nc(-n3c4ccccc4c4ccc(-c5ccc6c7ncccc7n(-c7ccccc7)c6c5)cc43)nc3ccccc23)cc1]

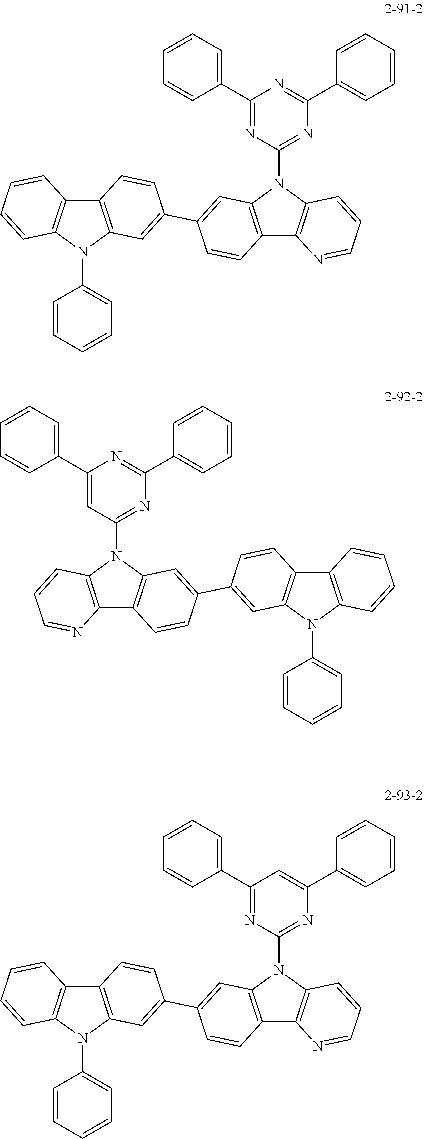 [SMILES: c1ccc(-c2nc(-c3ccccc3)nc(-n3c4cc(-c5ccc6c7ccccc7n(-c7ccccc7)c6c5)ccc4c4ncccc43)n2)cc1 ; c1ccc(-c2cc(-n3c4cc(-c5ccc6c7ccccc7n(-c7ccccc7)c6c5)ccc4c4ncccc43)nc(-c3ccccc3)n2)cc1 ; c1ccc(-c2cc(-c3ccccc3)nc(-n3c4cc(-c5ccc6c7ccccc7n(-c7ccccc7)c6c5)ccc4c4ncccc43)n2)cc1]

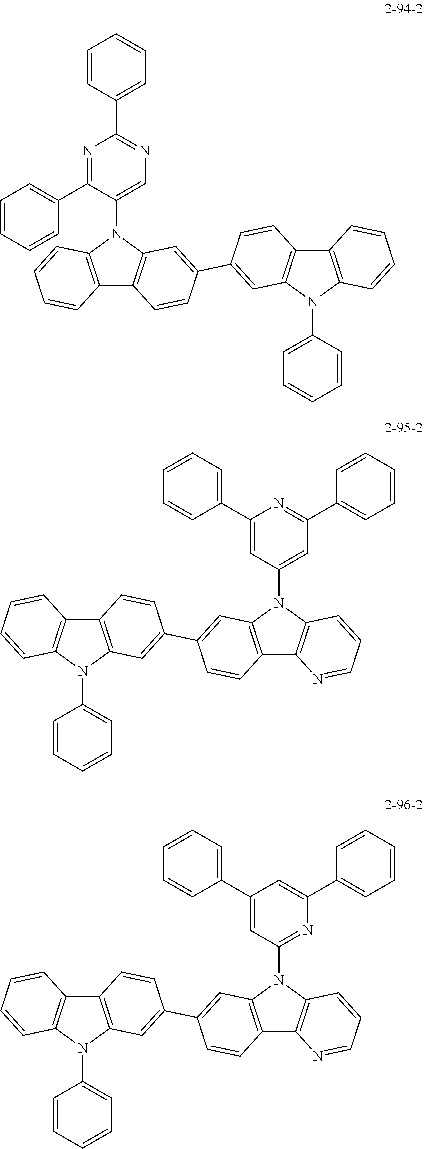 [SMILES: c1ccc(-c2ncc(-n3c4ccccc4c4ccc(-c5ccc6c7ccccc7n(-c7ccccc7)c6c5)cc43)c(-c3ccccc3)n2)cc1 ; c1ccc(-c2cc(-n3c4cc(-c5ccc6c7ccccc7n(-c7ccccc7)c6c5)ccc4c4ncccc43)cc(-c3ccccc3)n2)cc1 ; c1ccc(-c2cc(-c3ccccc3)nc(-n3c4cc(-c5ccc6c7ccccc7n(-c7ccccc7)c6c5)ccc4c4ncccc43)c2)cc1]

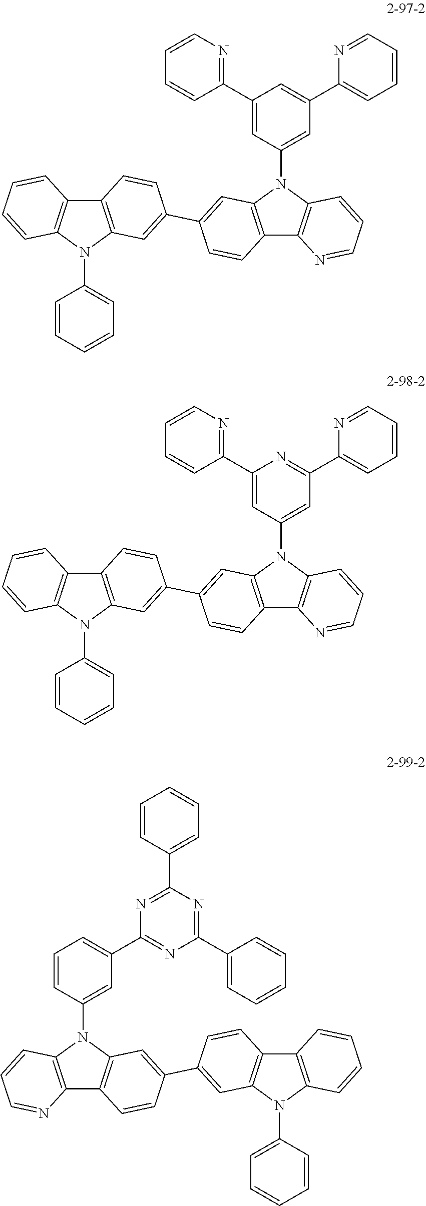 [SMILES: c1ccc(-n2c3ccccc3c3ccc(-c4ccc5c6ncccc6n(-c6cc(-c7ccccn7)cc(-c7ccccn7)c6)c5c4)cc32)cc1 ; c1ccc(-n2c3ccccc3c3ccc(-c4ccc5c6ncccc6n(-c6cc(-c7ccccn7)nc(-c7ccccn7)c6)c5c4)cc32)cc1 ; c1ccc(-c2nc(-c3ccccc3)nc(-c3cccc(-n4c5cc(-c6ccc7c8ccccc8n(-c8ccccc8)c7c6)ccc5c5ncccc54)c3)n2)cc1]

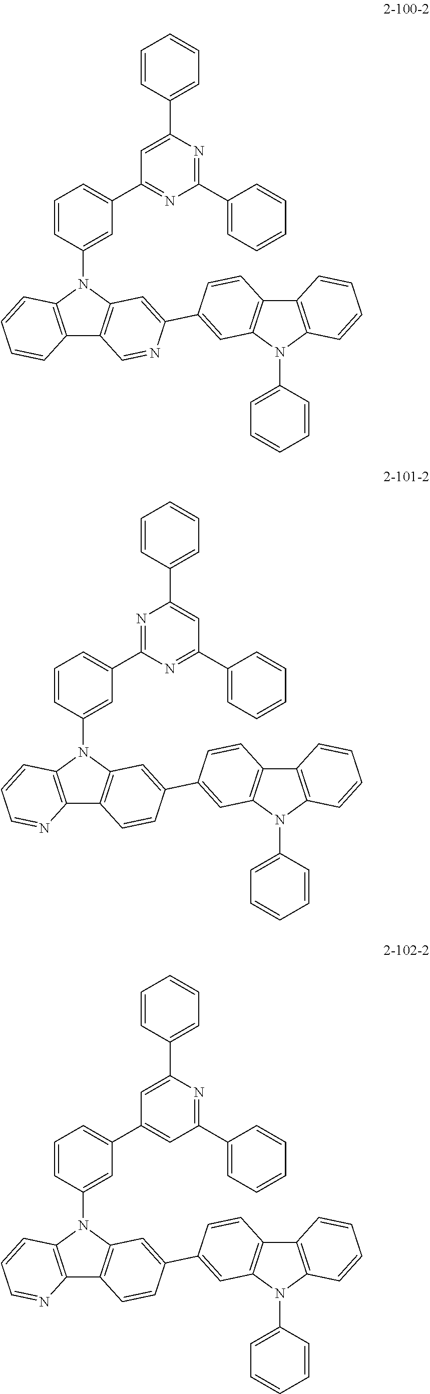 [SMILES: c1ccc(-c2cc(-c3cccc(-n4c5ccccc5c5cnc(-c6ccc7c8ccccc8n(-c8ccccc8)c7c6)cc54)c3)nc(-c3ccccc3)n2)cc1 ; c1ccc(-c2cc(-c3ccccc3)nc(-c3cccc(-n4c5cc(-c6ccc7c8ccccc8n(-c8ccccc8)c7c6)ccc5c5ncccc54)c3)n2)cc1 ; c1ccc(-c2cc(-c3cccc(-n4c5cc(-c6ccc7c8ccccc8n(-c8ccccc8)c7c6)ccc5c5ncccc54)c3)cc(-c3ccccc3)n2)cc1]

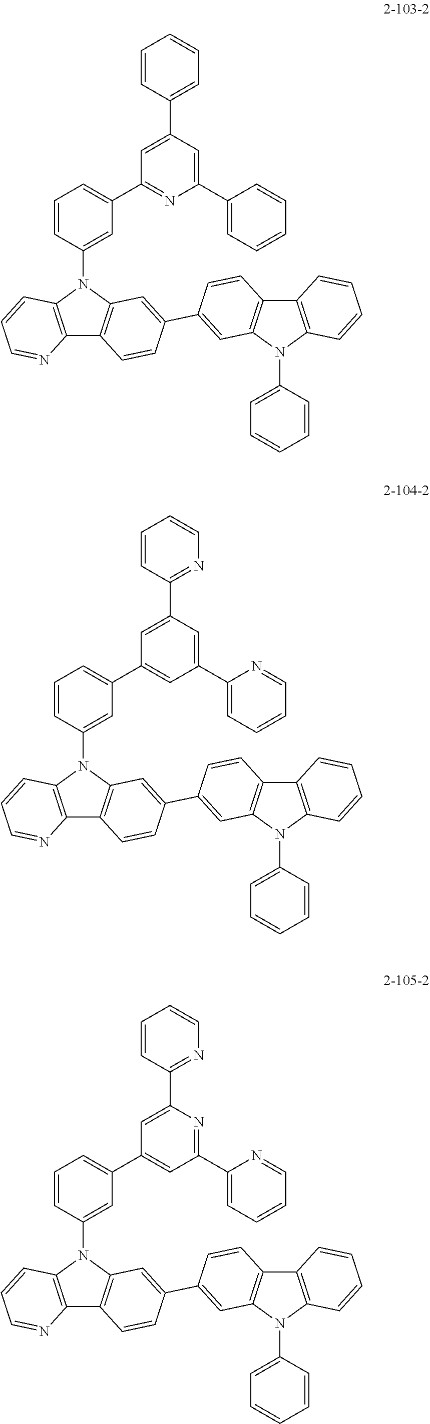 [SMILES: c1ccc(-c2cc(-c3ccccc3)nc(-c3cccc(-n4c5cc(-c6ccc7c8ccccc8n(-c8ccccc8)c7c6)ccc5c5ncccc54)c3)c2)cc1 ; c1ccc(-n2c3ccccc3c3ccc(-c4ccc5c6ncccc6n(-c6cccc(-c7cc(-c8ccccn8)cc(-c8ccccn8)c7)c6)c5c4)cc32)cc1 ; c1ccc(-n2c3ccccc3c3ccc(-c4ccc5c6ncccc6n(-c6cccc(-c7cc(-c8ccccn8)nc(-c8ccccn8)c7)c6)c5c4)cc32)cc1]

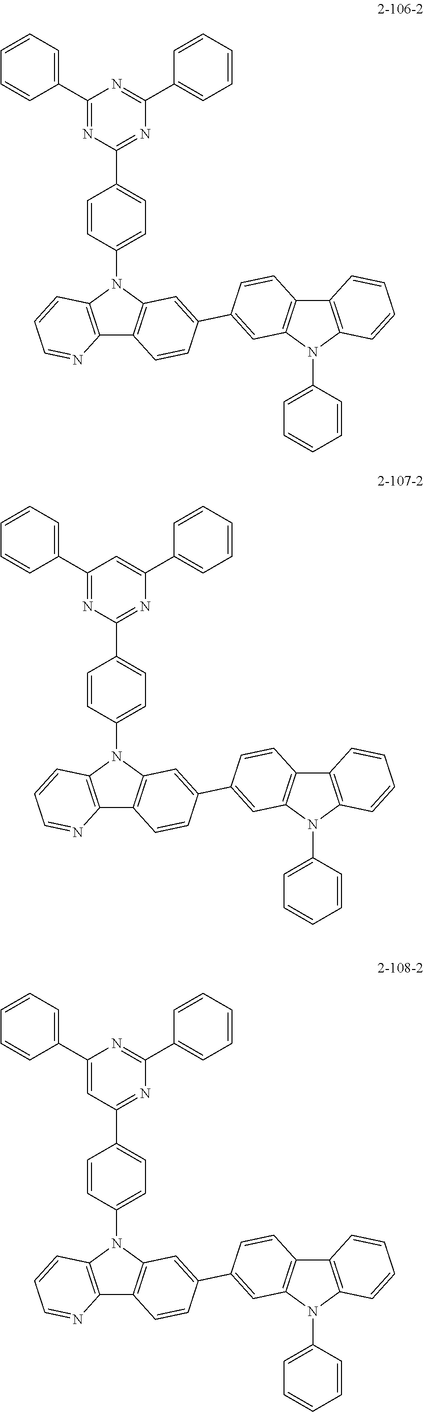 [SMILES: c1ccc(-c2nc(-c3ccccc3)nc(-c3ccc(-n4c5cc(-c6ccc7c8ccccc8n(-c8ccccc8)c7c6)ccc5c5ncccc54)cc3)n2)cc1 ; c1ccc(-c2cc(-c3ccccc3)nc(-c3ccc(-n4c5cc(-c6ccc7c8ccccc8n(-c8ccccc8)c7c6)ccc5c5ncccc54)cc3)n2)cc1 ; c1ccc(-c2cc(-c3ccc(-n4c5cc(-c6ccc7c8ccccc8n(-c8ccccc8)c7c6)ccc5c5ncccc54)cc3)nc(-c3ccccc3)n2)cc1]

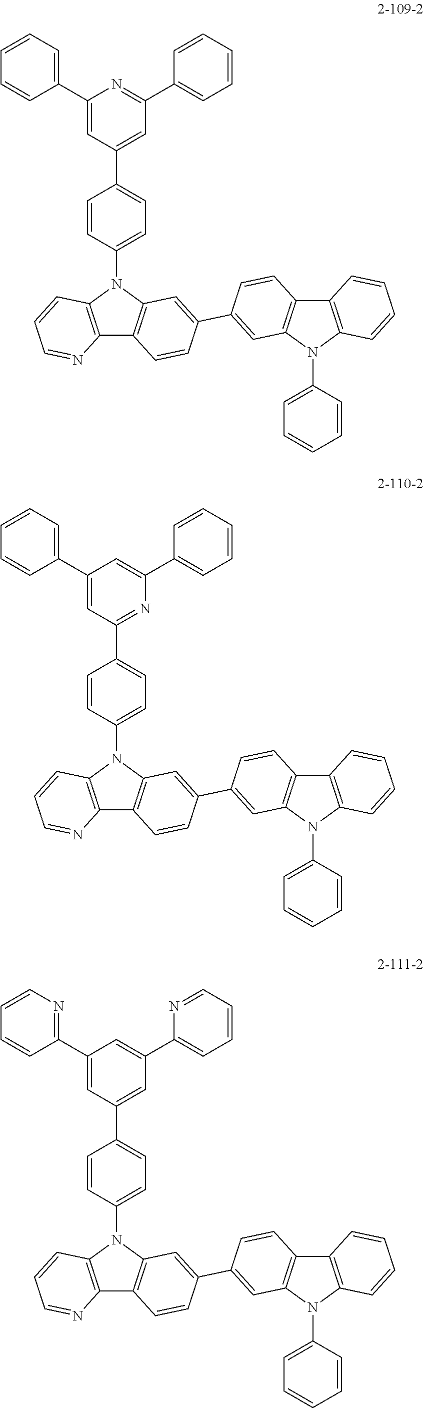 [SMILES: c1ccc(-c2cc(-c3ccc(-n4c5cc(-c6ccc7c8ccccc8n(-c8ccccc8)c7c6)ccc5c5ncccc54)cc3)cc(-c3ccccc3)n2)cc1 ; c1ccc(-c2cc(-c3ccccc3)nc(-c3ccc(-n4c5cc(-c6ccc7c8ccccc8n(-c8ccccc8)c7c6)ccc5c5ncccc54)cc3)c2)cc1 ; c1ccc(-n2c3ccccc3c3ccc(-c4ccc5c6ncccc6n(-c6ccc(-c7cc(-c8ccccn8)cc(-c8ccccn8)c7)cc6)c5c4)cc32)cc1]

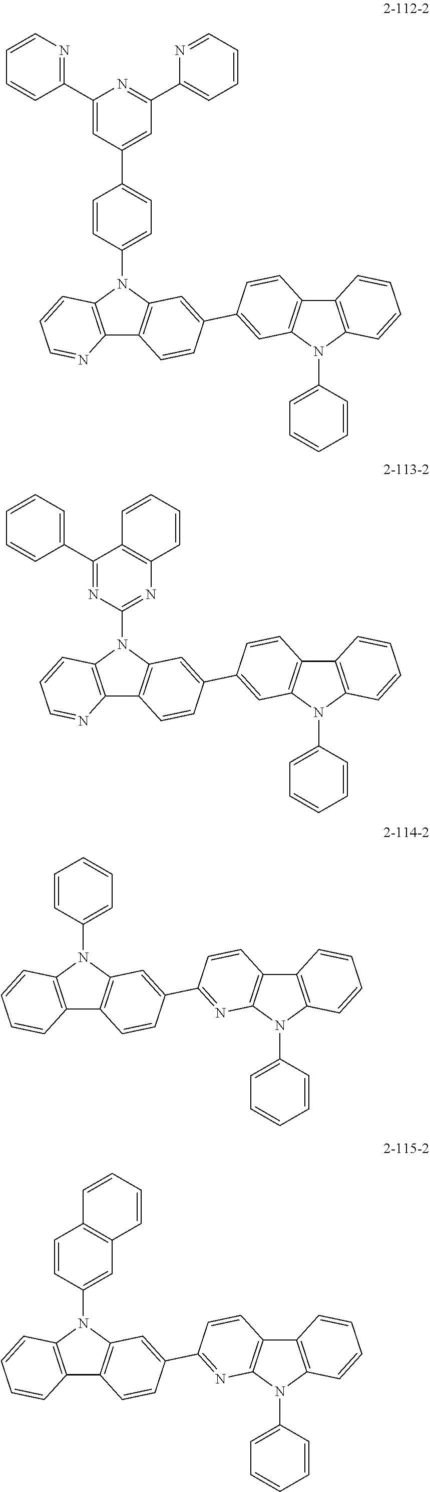 [SMILES: c1ccc(-n2c3ccccc3c3ccc(-c4ccc5c6ncccc6n(-c6ccc(-c7cc(-c8ccccn8)nc(-c8ccccn8)c7)cc6)c5c4)cc32)cc1 ; c1ccc(-c2nc(-n3c4cc(-c5ccc6c7ccccc7n(-c7ccccc7)c6c5)ccc4c4ncccc43)nc3ccccc23)cc1 ; c1ccc(-n2c3ccccc3c3ccc(-c4ccc5c6ccccc6n(-c6ccccc6)c5n4)cc32)cc1 ; c1ccc(-n2c3ccccc3c3ccc(-c4ccc5c6ccccc6n(-c6ccc7ccccc7c6)c5c4)nc32)cc1]

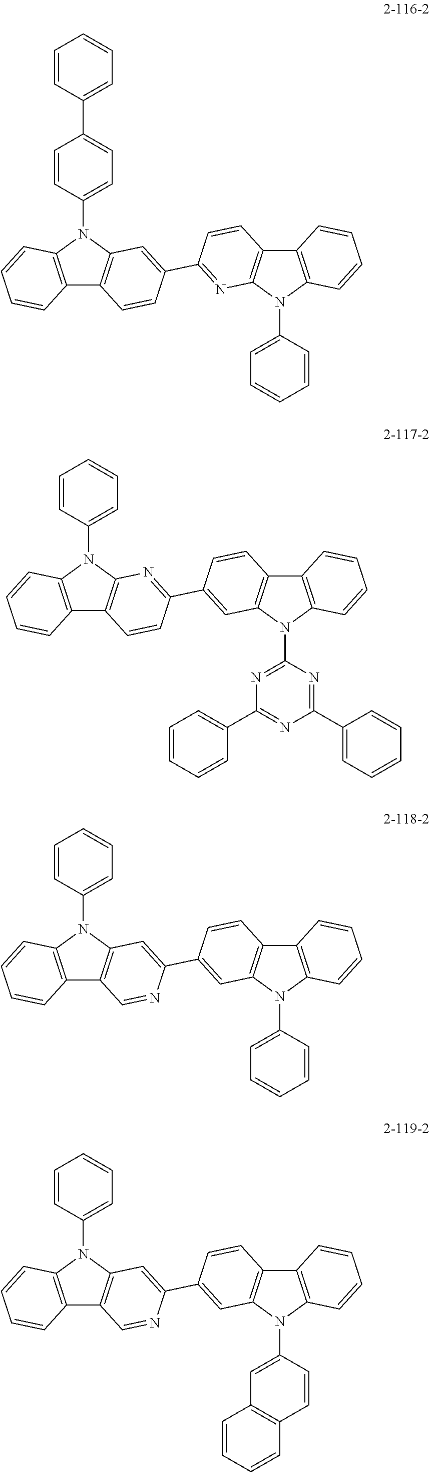 [SMILES: c1ccc(-c2ccc(-n3c4ccccc4c4ccc(-c5ccc6c7ccccc7n(-c7ccccc7)c6n5)cc43)cc2)cc1 ; c1ccc(-c2nc(-c3ccccc3)nc(-n3c4ccccc4c4ccc(-c5ccc6c7ccccc7n(-c7ccccc7)c6n5)cc43)n2)cc1 ; c1ccc(-n2c3ccccc3c3ccc(-c4cc5c(cn4)c4ccccc4n5-c4ccccc4)cc32)cc1 ; c1ccc(-n2c3ccccc3c3cnc(-c4ccc5c6ccccc6n(-c6ccc7ccccc7c6)c5c4)cc32)cc1]

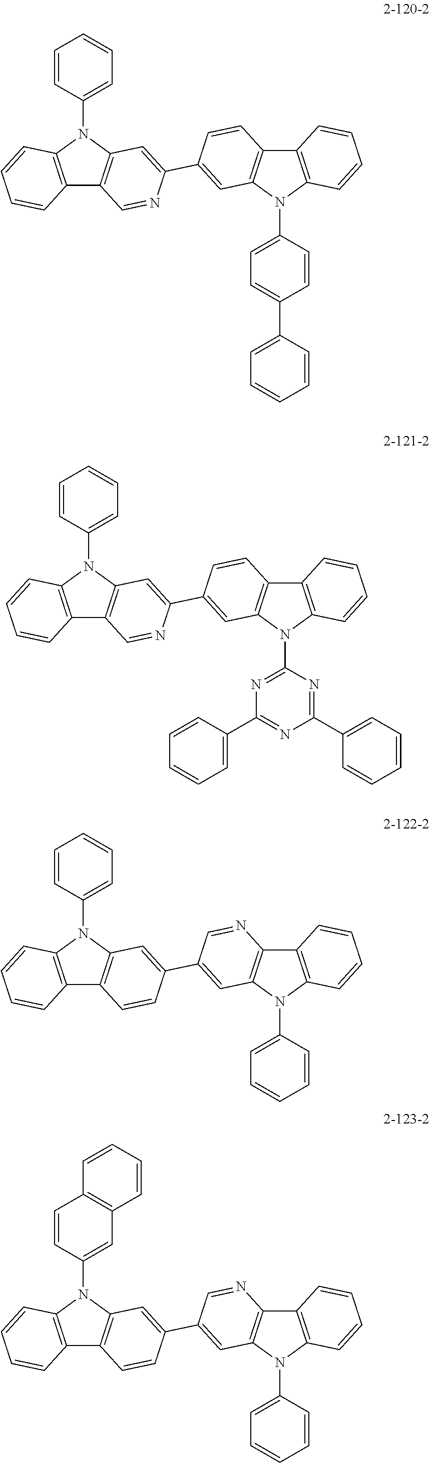 [SMILES: c1ccc(-c2ccc(-n3c4ccccc4c4ccc(-c5cc6c(cn5)c5ccccc5n6-c5ccccc5)cc43)cc2)cc1 ; c1ccc(-c2nc(-c3ccccc3)nc(-n3c4ccccc4c4ccc(-c5cc6c(cn5)c5ccccc5n6-c5ccccc5)cc43)n2)cc1 ; c1ccc(-n2c3ccccc3c3ccc(-c4cnc5c6ccccc6n(-c6ccccc6)c5c4)cc32)cc1 ; c1ccc(-n2c3ccccc3c3ncc(-c4ccc5c6ccccc6n(-c6ccc7ccccc7c6)c5c4)cc32)cc1]

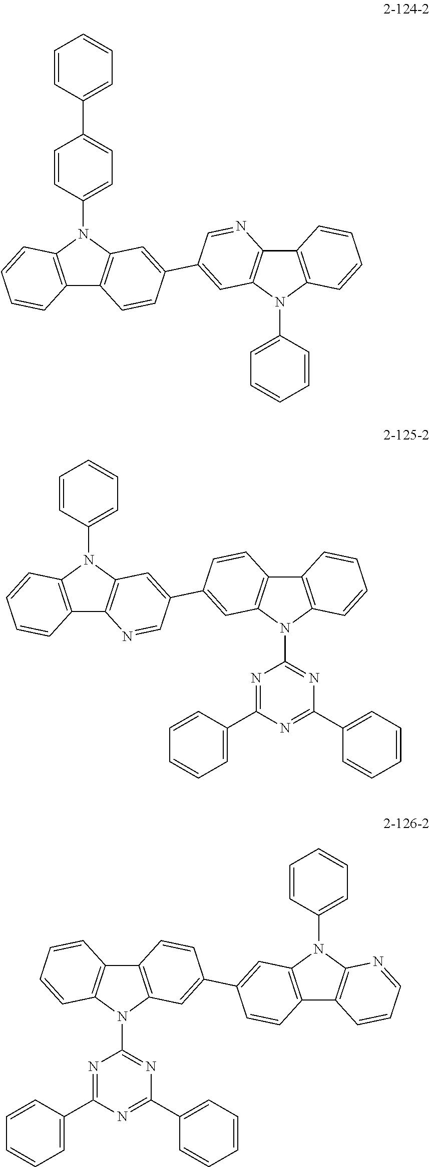 [SMILES: c1ccc(-c2ccc(-n3c4ccccc4c4ccc(-c5cnc6c7ccccc7n(-c7ccccc7)c6c5)cc43)cc2)cc1 ; c1ccc(-c2nc(-c3ccccc3)nc(-n3c4ccccc4c4ccc(-c5cnc6c7ccccc7n(-c7ccccc7)c6c5)cc43)n2)cc1 ; c1ccc(-c2nc(-c3ccccc3)nc(-n3c4ccccc4c4ccc(-c5ccc6c7cccnc7n(-c7ccccc7)c6c5)cc43)n2)cc1]

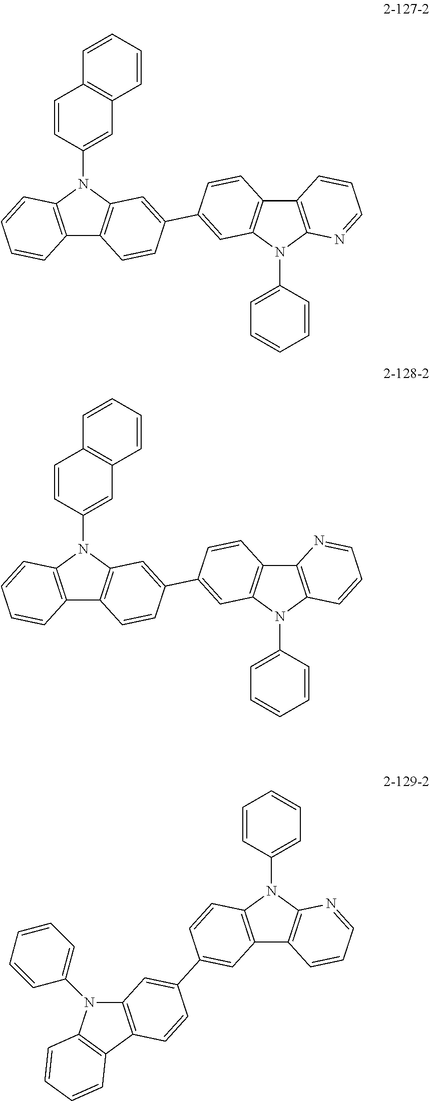 [SMILES: c1ccc(-n2c3cc(-c4ccc5c6ccccc6n(-c6ccc7ccccc7c6)c5c4)ccc3c3cccnc32)cc1 ; c1ccc(-n2c3cc(-c4ccc5c6ccccc6n(-c6ccc7ccccc7c6)c5c4)ccc3c3ncccc32)cc1 ; c1ccc(-n2c3ccccc3c3ccc(-c4ccc5c(c4)c4cccnc4n5-c4ccccc4)cc32)cc1]

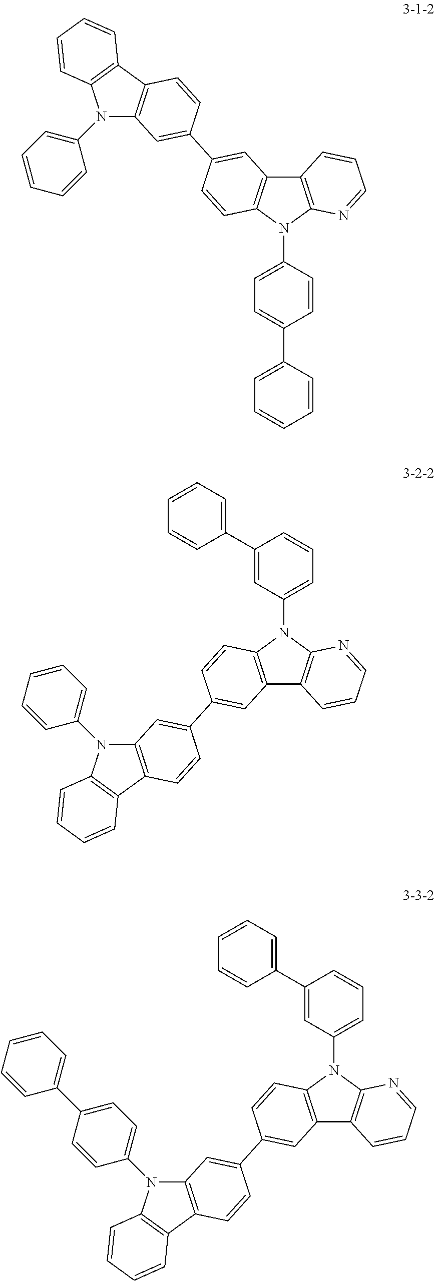 [SMILES: c1ccc(-c2ccc(-n3c4ccc(-c5ccc6c7ccccc7n(-c7ccccc7)c6c5)cc4c4cccnc43)cc2)cc1 ; c1ccc(-c2cccc(-n3c4ccc(-c5ccc6c7ccccc7n(-c7ccccc7)c6c5)cc4c4cccnc43)c2)cc1 ; c1ccc(-c2ccc(-n3c4ccccc4c4ccc(-c5ccc6c(c5)c5cccnc5n6-c5cccc(-c6ccccc6)c5)cc43)cc2)cc1]

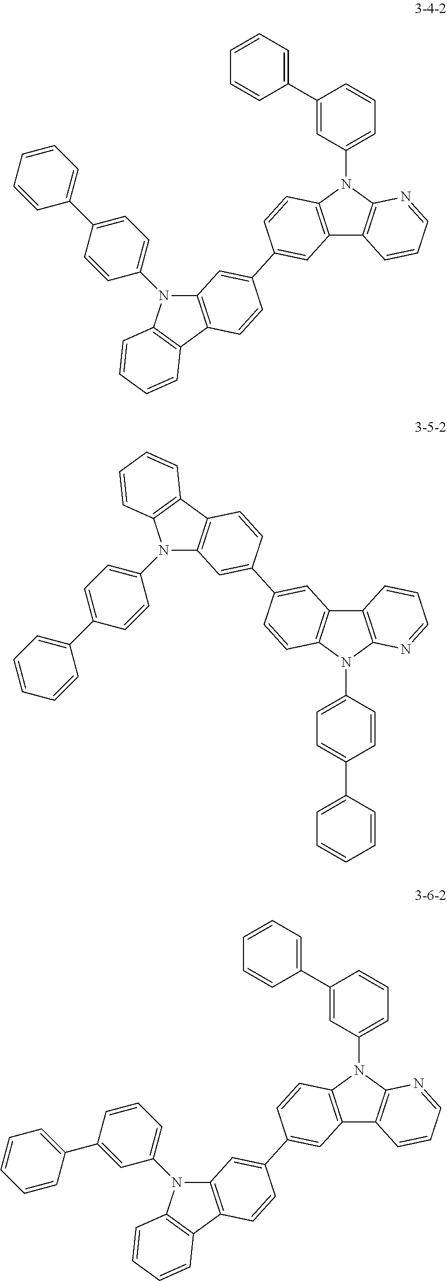 [SMILES: c1ccc(-c2ccc(-n3c4ccccc4c4ccc(-c5ccc6c(c5)c5cccnc5n6-c5cccc(-c6ccccc6)c5)cc43)cc2)cc1 ; c1ccc(-c2ccc(-n3c4ccccc4c4ccc(-c5ccc6c(c5)c5cccnc5n6-c5ccc(-c6ccccc6)cc5)cc43)cc2)cc1 ; c1ccc(-c2cccc(-n3c4ccccc4c4ccc(-c5ccc6c(c5)c5cccnc5n6-c5cccc(-c6ccccc6)c5)cc43)c2)cc1]

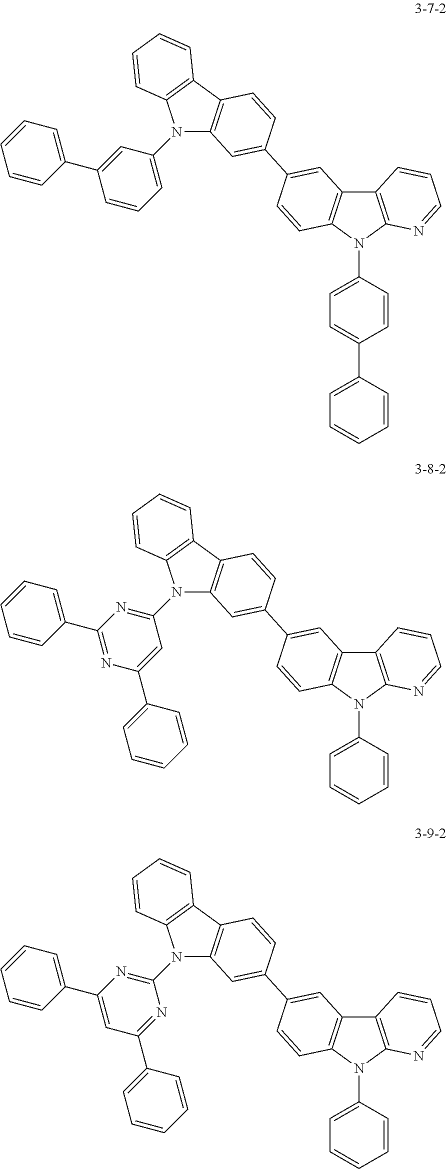 [SMILES: c1ccc(-c2ccc(-n3c4ccc(-c5ccc6c7ccccc7n(-c7cccc(-c8ccccc8)c7)c6c5)cc4c4cccnc43)cc2)cc1 ; c1ccc(-c2cc(-n3c4ccccc4c4ccc(-c5ccc6c(c5)c5cccnc5n6-c5ccccc5)cc43)nc(-c3ccccc3)n2)cc1 ; c1ccc(-c2cc(-c3ccccc3)nc(-n3c4ccccc4c4ccc(-c5ccc6c(c5)c5cccnc5n6-c5ccccc5)cc43)n2)cc1]

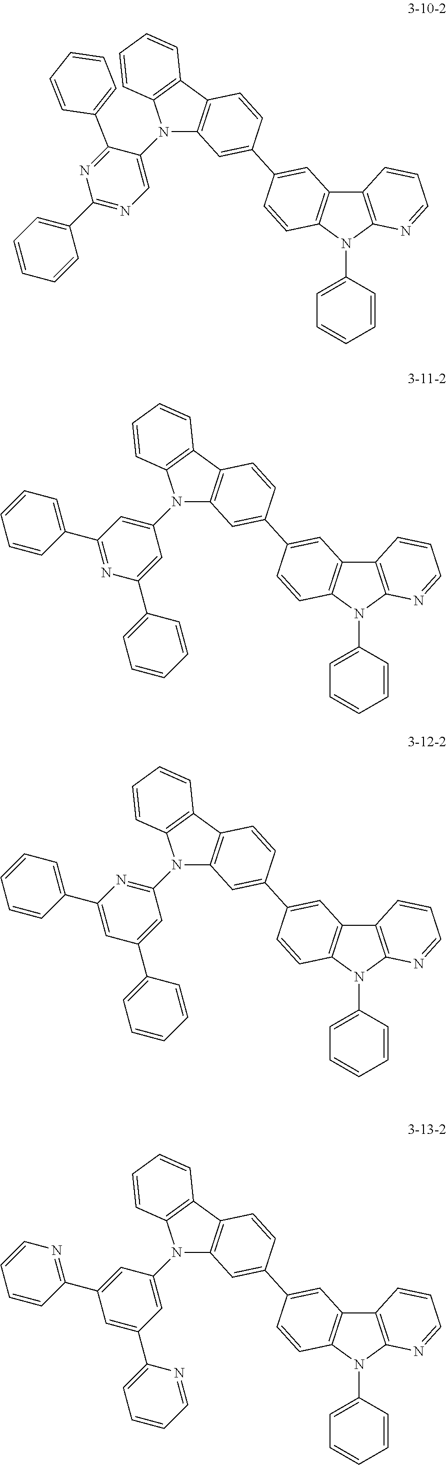 [SMILES: c1ccc(-c2ncc(-n3c4ccccc4c4ccc(-c5ccc6c(c5)c5cccnc5n6-c5ccccc5)cc43)c(-c3ccccc3)n2)cc1 ; c1ccc(-c2cc(-n3c4ccccc4c4ccc(-c5ccc6c(c5)c5cccnc5n6-c5ccccc5)cc43)cc(-c3ccccc3)n2)cc1 ; c1ccc(-c2cc(-c3ccccc3)nc(-n3c4ccccc4c4ccc(-c5ccc6c(c5)c5cccnc5n6-c5ccccc5)cc43)c2)cc1 ; c1ccc(-n2c3ccc(-c4ccc5c6ccccc6n(-c6cc(-c7ccccn7)cc(-c7ccccn7)c6)c5c4)cc3c3cccnc32)cc1]

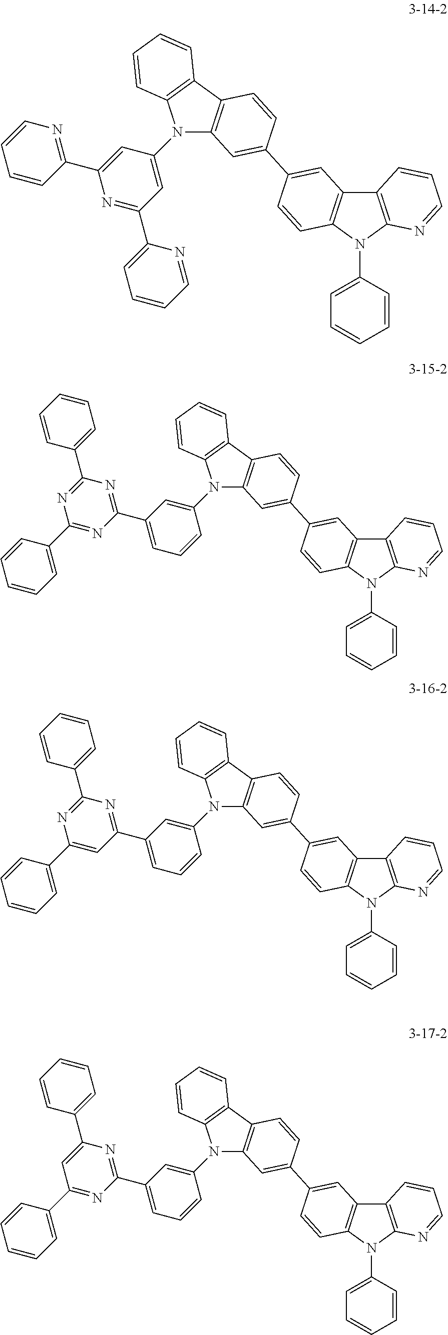 [SMILES: c1ccc(-n2c3ccc(-c4ccc5c6ccccc6n(-c6cc(-c7ccccn7)nc(-c7ccccn7)c6)c5c4)cc3c3cccnc32)cc1 ; c1ccc(-c2nc(-c3ccccc3)nc(-c3cccc(-n4c5ccccc5c5ccc(-c6ccc7c(c6)c6cccnc6n7-c6ccccc6)cc54)c3)n2)cc1 ; c1ccc(-c2cc(-c3cccc(-n4c5ccccc5c5ccc(-c6ccc7c(c6)c6cccnc6n7-c6ccccc6)cc54)c3)nc(-c3ccccc3)n2)cc1 ; c1ccc(-c2cc(-c3ccccc3)nc(-c3cccc(-n4c5ccccc5c5ccc(-c6ccc7c(c6)c6cccnc6n7-c6ccccc6)cc54)c3)n2)cc1]

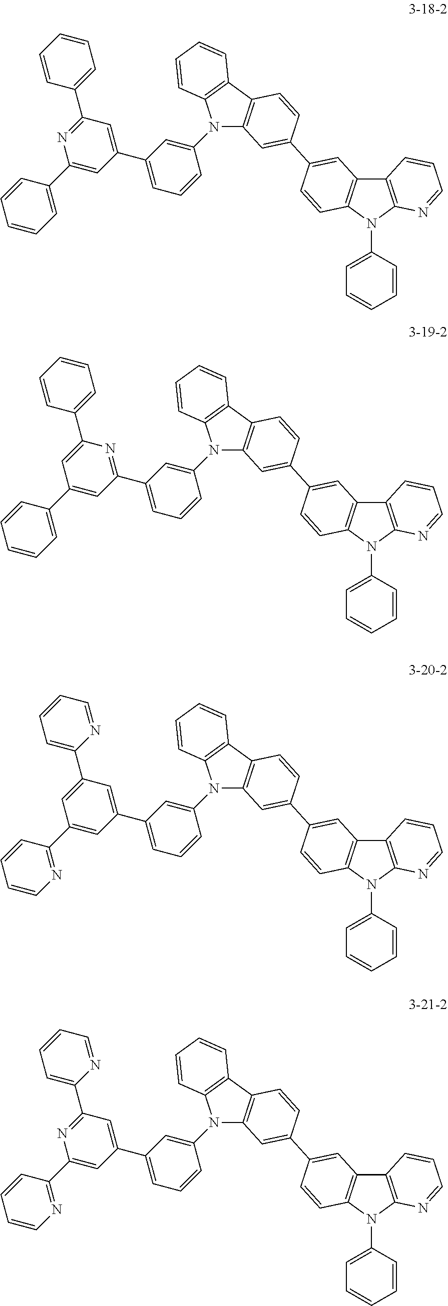 [SMILES: c1ccc(-c2cc(-c3cccc(-n4c5ccccc5c5ccc(-c6ccc7c(c6)c6cccnc6n7-c6ccccc6)cc54)c3)cc(-c3ccccc3)n2)cc1 ; c1ccc(-c2cc(-c3ccccc3)nc(-c3cccc(-n4c5ccccc5c5ccc(-c6ccc7c(c6)c6cccnc6n7-c6ccccc6)cc54)c3)c2)cc1 ; c1ccc(-n2c3ccc(-c4ccc5c6ccccc6n(-c6cccc(-c7cc(-c8ccccn8)cc(-c8ccccn8)c7)c6)c5c4)cc3c3cccnc32)cc1 ; c1ccc(-n2c3ccc(-c4ccc5c6ccccc6n(-c6cccc(-c7cc(-c8ccccn8)nc(-c8ccccn8)c7)c6)c5c4)cc3c3cccnc32)cc1]

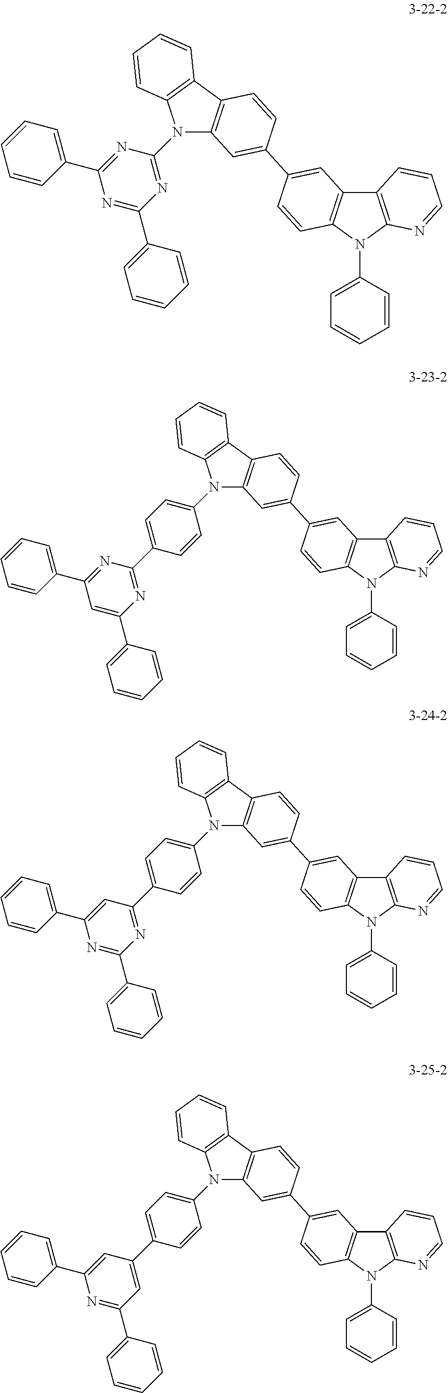 [SMILES: c1ccc(-c2nc(-c3ccccc3)nc(-n3c4ccccc4c4ccc(-c5ccc6c(c5)c5cccnc5n6-c5ccccc5)cc43)n2)cc1 ; c1ccc(-c2cc(-c3ccccc3)nc(-c3ccc(-n4c5ccccc5c5ccc(-c6ccc7c(c6)c6cccnc6n7-c6ccccc6)cc54)cc3)n2)cc1 ; c1ccc(-c2cc(-c3ccc(-n4c5ccccc5c5ccc(-c6ccc7c(c6)c6cccnc6n7-c6ccccc6)cc54)cc3)nc(-c3ccccc3)n2)cc1 ; c1ccc(-c2cc(-c3ccc(-n4c5ccccc5c5ccc(-c6ccc7c(c6)c6cccnc6n7-c6ccccc6)cc54)cc3)cc(-c3ccccc3)n2)cc1]

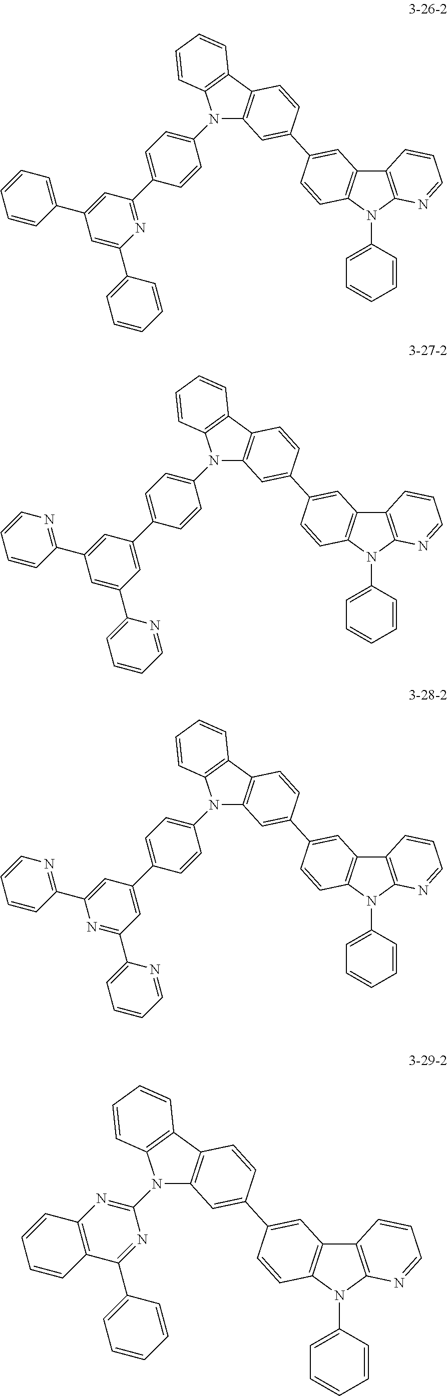 [SMILES: c1ccc(-c2cc(-c3ccccc3)nc(-c3ccc(-n4c5ccccc5c5ccc(-c6ccc7c(c6)c6cccnc6n7-c6ccccc6)cc54)cc3)c2)cc1 ; c1ccc(-n2c3ccc(-c4ccc5c6ccccc6n(-c6ccc(-c7cc(-c8ccccn8)cc(-c8ccccn8)c7)cc6)c5c4)cc3c3cccnc32)cc1 ; c1ccc(-n2c3ccc(-c4ccc5c6ccccc6n(-c6ccc(-c7cc(-c8ccccn8)nc(-c8ccccn8)c7)cc6)c5c4)cc3c3cccnc32)cc1 ; c1ccc(-c2nc(-n3c4ccccc4c4ccc(-c5ccc6c(c5)c5cccnc5n6-c5ccccc5)cc43)nc3ccccc23)cc1]

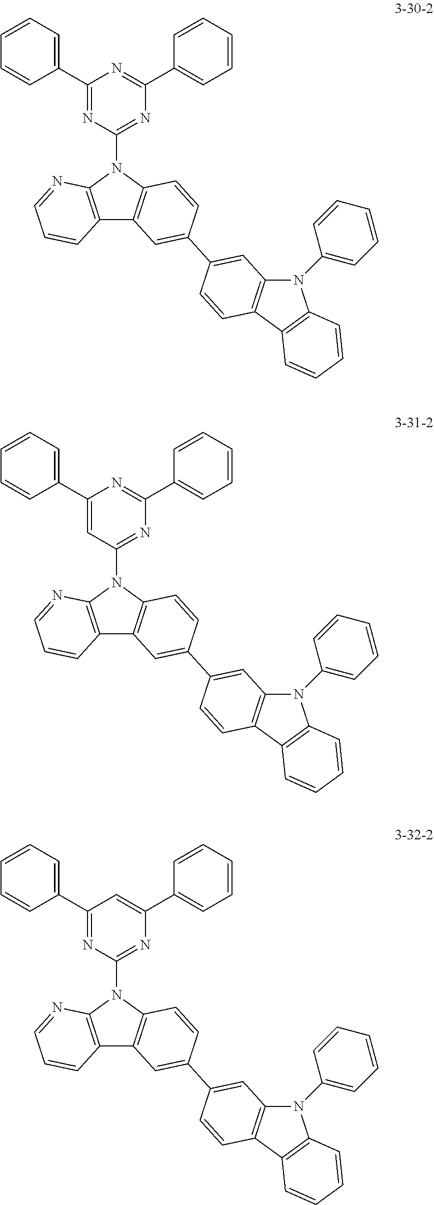 [SMILES: c1ccc(-c2nc(-c3ccccc3)nc(-n3c4ccc(-c5ccc6c7ccccc7n(-c7ccccc7)c6c5)cc4c4cccnc43)n2)cc1 ; c1ccc(-c2cc(-n3c4ccc(-c5ccc6c7ccccc7n(-c7ccccc7)c6c5)cc4c4cccnc43)nc(-c3ccccc3)n2)cc1 ; c1ccc(-c2cc(-c3ccccc3)nc(-n3c4ccc(-c5ccc6c7ccccc7n(-c7ccccc7)c6c5)cc4c4cccnc43)n2)cc1]

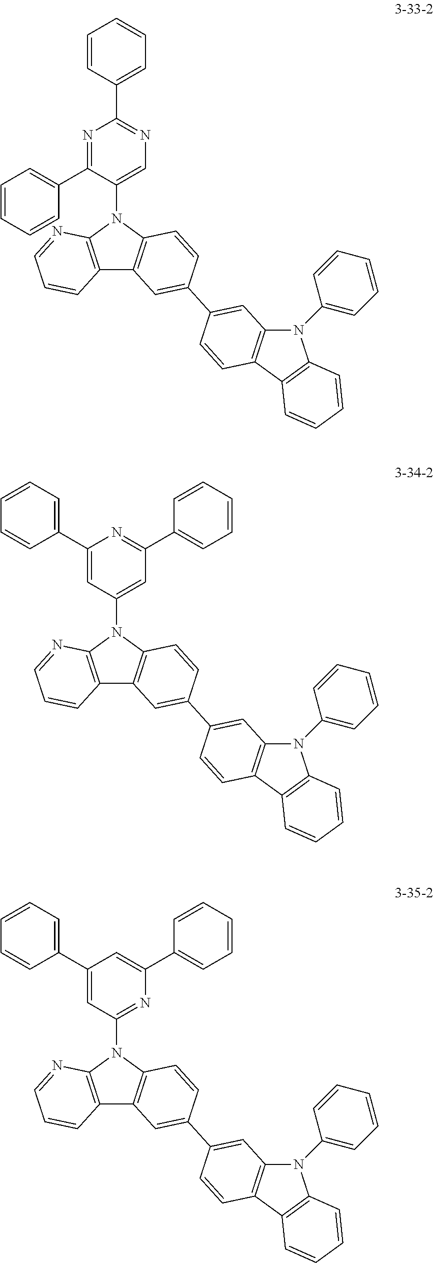 [SMILES: c1ccc(-c2ncc(-n3c4ccc(-c5ccc6c7ccccc7n(-c7ccccc7)c6c5)cc4c4cccnc43)c(-c3ccccc3)n2)cc1 ; c1ccc(-c2cc(-n3c4ccc(-c5ccc6c7ccccc7n(-c7ccccc7)c6c5)cc4c4cccnc43)cc(-c3ccccc3)n2)cc1 ; c1ccc(-c2cc(-c3ccccc3)nc(-n3c4ccc(-c5ccc6c7ccccc7n(-c7ccccc7)c6c5)cc4c4cccnc43)c2)cc1]

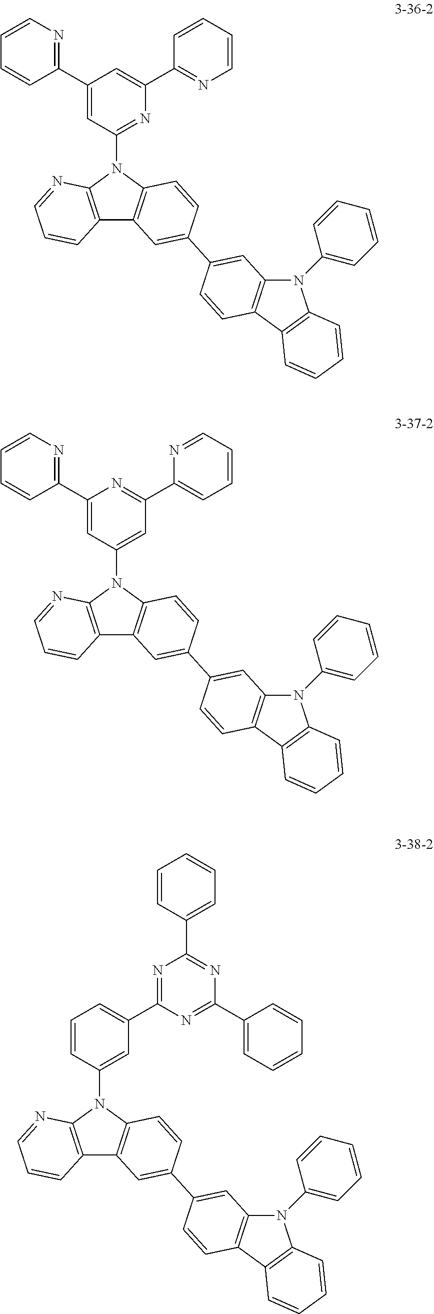 [SMILES: c1ccc(-n2c3ccccc3c3ccc(-c4ccc5c(c4)c4cccnc4n5-c4cc(-c5ccccn5)cc(-c5ccccn5)n4)cc32)cc1 ; c1ccc(-n2c3ccccc3c3ccc(-c4ccc5c(c4)c4cccnc4n5-c4cc(-c5ccccn5)nc(-c5ccccn5)c4)cc32)cc1 ; c1ccc(-c2nc(-c3ccccc3)nc(-c3cccc(-n4c5ccc(-c6ccc7c8ccccc8n(-c8ccccc8)c7c6)cc5c5cccnc54)c3)n2)cc1]

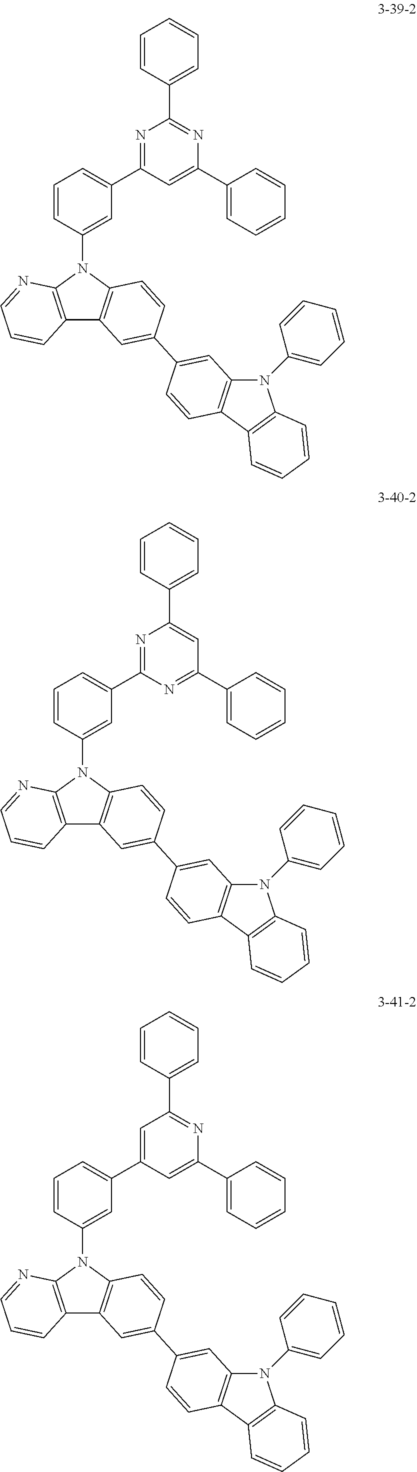 [SMILES: c1ccc(-c2cc(-c3cccc(-n4c5ccc(-c6ccc7c8ccccc8n(-c8ccccc8)c7c6)cc5c5cccnc54)c3)nc(-c3ccccc3)n2)cc1 ; c1ccc(-c2cc(-c3ccccc3)nc(-c3cccc(-n4c5ccc(-c6ccc7c8ccccc8n(-c8ccccc8)c7c6)cc5c5cccnc54)c3)n2)cc1 ; c1ccc(-c2cc(-c3cccc(-n4c5ccc(-c6ccc7c8ccccc8n(-c8ccccc8)c7c6)cc5c5cccnc54)c3)cc(-c3ccccc3)n2)cc1]

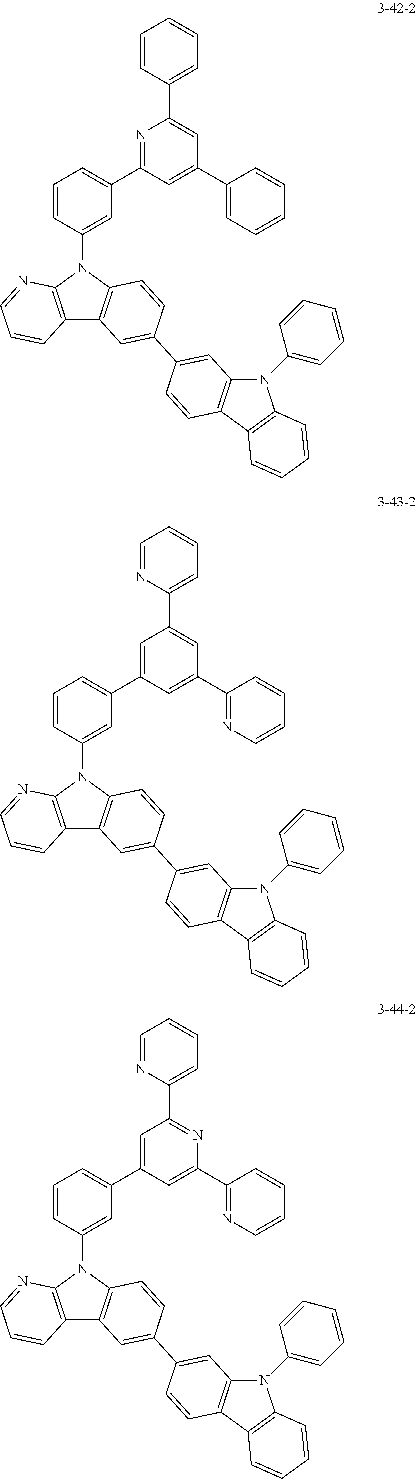 [SMILES: c1ccc(-c2cc(-c3ccccc3)nc(-c3cccc(-n4c5ccc(-c6ccc7c8ccccc8n(-c8ccccc8)c7c6)cc5c5cccnc54)c3)c2)cc1 ; c1ccc(-n2c3ccccc3c3ccc(-c4ccc5c(c4)c4cccnc4n5-c4cccc(-c5cc(-c6ccccn6)cc(-c6ccccn6)c5)c4)cc32)cc1 ; c1ccc(-n2c3ccccc3c3ccc(-c4ccc5c(c4)c4cccnc4n5-c4cccc(-c5cc(-c6ccccn6)nc(-c6ccccn6)c5)c4)cc32)cc1]

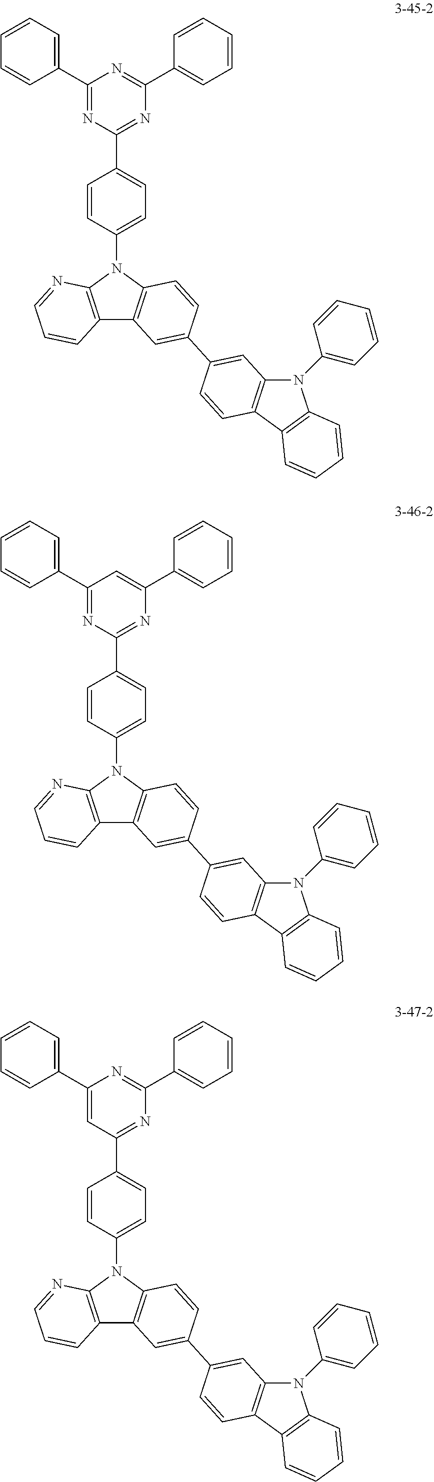 [SMILES: c1ccc(-c2nc(-c3ccccc3)nc(-c3ccc(-n4c5ccc(-c6ccc7c8ccccc8n(-c8ccccc8)c7c6)cc5c5cccnc54)cc3)n2)cc1 ; c1ccc(-c2cc(-c3ccccc3)nc(-c3ccc(-n4c5ccc(-c6ccc7c8ccccc8n(-c8ccccc8)c7c6)cc5c5cccnc54)cc3)n2)cc1 ; c1ccc(-c2cc(-c3ccc(-n4c5ccc(-c6ccc7c8ccccc8n(-c8ccccc8)c7c6)cc5c5cccnc54)cc3)nc(-c3ccccc3)n2)cc1]

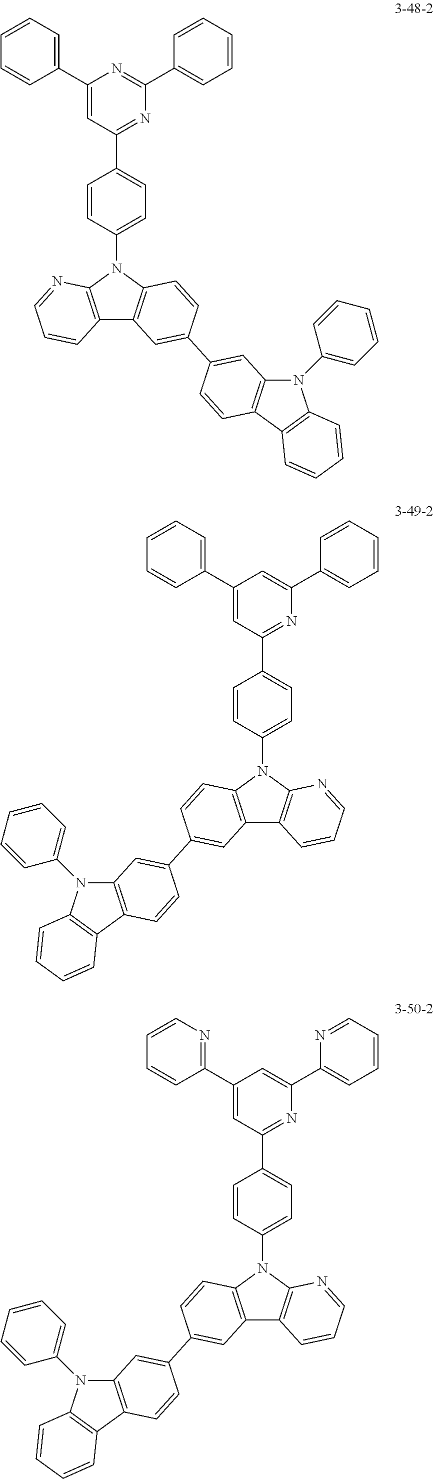 [SMILES: c1ccc(-c2cc(-c3ccc(-n4c5ccc(-c6ccc7c8ccccc8n(-c8ccccc8)c7c6)cc5c5cccnc54)cc3)nc(-c3ccccc3)n2)cc1 ; c1ccc(-c2cc(-c3ccccc3)nc(-c3ccc(-n4c5ccc(-c6ccc7c8ccccc8n(-c8ccccc8)c7c6)cc5c5cccnc54)cc3)c2)cc1 ; c1ccc(-n2c3ccccc3c3ccc(-c4ccc5c(c4)c4cccnc4n5-c4ccc(-c5cc(-c6ccccn6)cc(-c6ccccn6)n5)cc4)cc32)cc1]

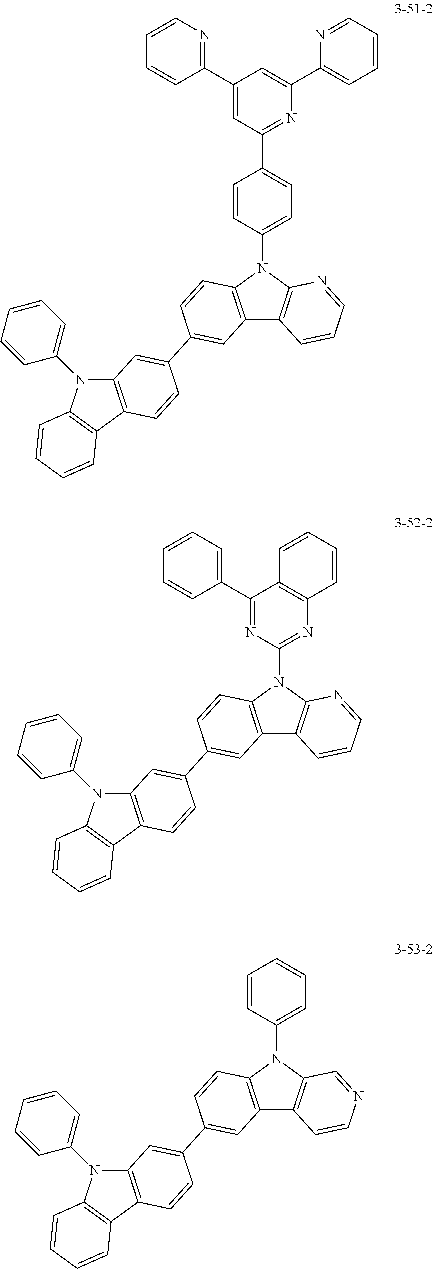 [SMILES: c1ccc(-n2c3ccccc3c3ccc(-c4ccc5c(c4)c4cccnc4n5-c4ccc(-c5cc(-c6ccccn6)cc(-c6ccccn6)n5)cc4)cc32)cc1 ; c1ccc(-c2nc(-n3c4ccc(-c5ccc6c7ccccc7n(-c7ccccc7)c6c5)cc4c4cccnc43)nc3ccccc23)cc1 ; c1ccc(-n2c3ccc(-c4ccc5c6ccccc6n(-c6ccccc6)c5c4)cc3c3ccncc32)cc1]

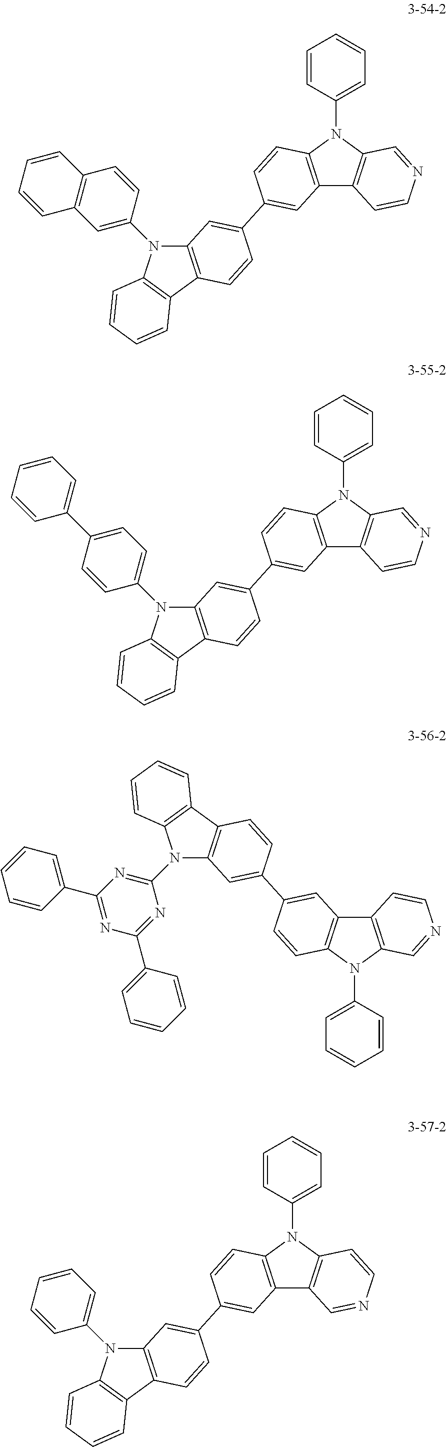 [SMILES: c1ccc(-n2c3ccc(-c4ccc5c6ccccc6n(-c6ccc7ccccc7c6)c5c4)cc3c3ccncc32)cc1 ; c1ccc(-c2ccc(-n3c4ccccc4c4ccc(-c5ccc6c(c5)c5ccncc5n6-c5ccccc5)cc43)cc2)cc1 ; c1ccc(-c2nc(-c3ccccc3)nc(-n3c4ccccc4c4ccc(-c5ccc6c(c5)c5ccncc5n6-c5ccccc5)cc43)n2)cc1 ; c1ccc(-n2c3ccncc3c3cc(-c4ccc5c6ccccc6n(-c6ccccc6)c5c4)ccc32)cc1]

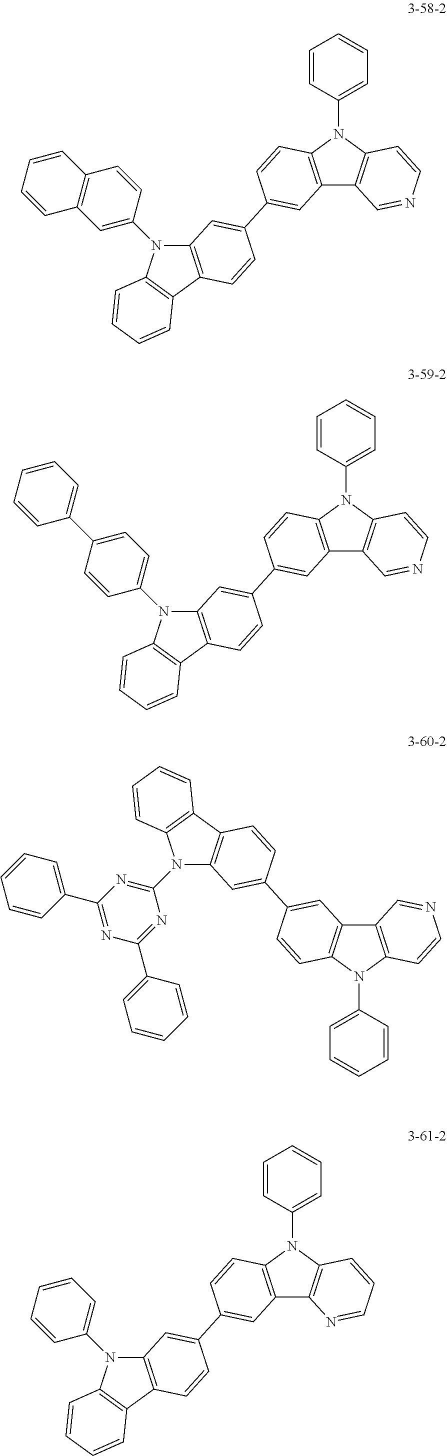 [SMILES: c1ccc(-n2c3ccncc3c3cc(-c4ccc5c6ccccc6n(-c6ccc7ccccc7c6)c5c4)ccc32)cc1 ; c1ccc(-c2ccc(-n3c4ccccc4c4ccc(-c5ccc6c(c5)c5cnccc5n6-c5ccccc5)cc43)cc2)cc1 ; c1ccc(-c2nc(-c3ccccc3)nc(-n3c4ccccc4c4ccc(-c5ccc6c(c5)c5cnccc5n6-c5ccccc5)cc43)n2)cc1 ; c1ccc(-n2c3ccccc3c3ccc(-c4ccc5c(c4)c4ncccc4n5-c4ccccc4)cc32)cc1]

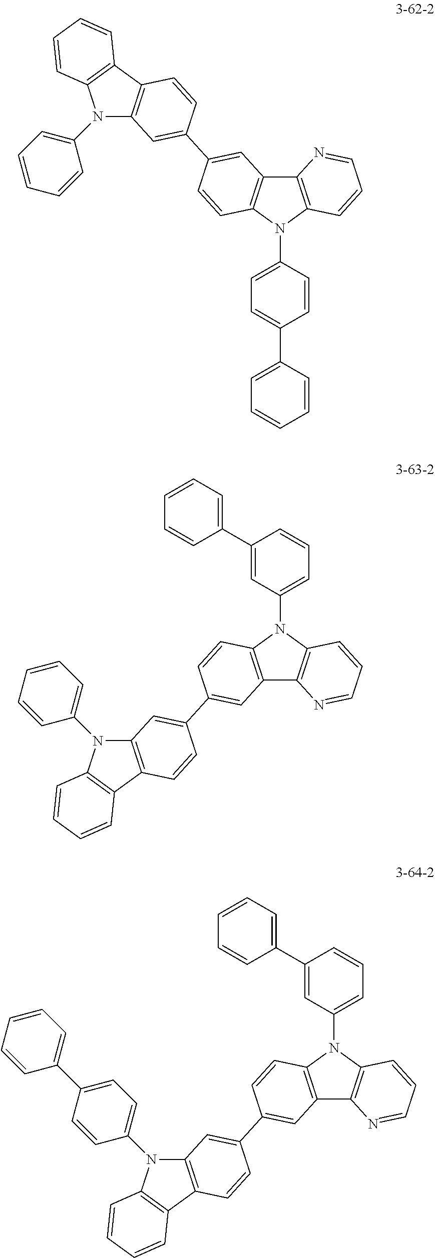 [SMILES: c1ccc(-c2ccc(-n3c4ccc(-c5ccc6c7ccccc7n(-c7ccccc7)c6c5)cc4c4ncccc43)cc2)cc1 ; c1ccc(-c2cccc(-n3c4ccc(-c5ccc6c7ccccc7n(-c7ccccc7)c6c5)cc4c4ncccc43)c2)cc1 ; c1ccc(-c2ccc(-n3c4ccccc4c4ccc(-c5ccc6c(c5)c5ncccc5n6-c5cccc(-c6ccccc6)c5)cc43)cc2)cc1]

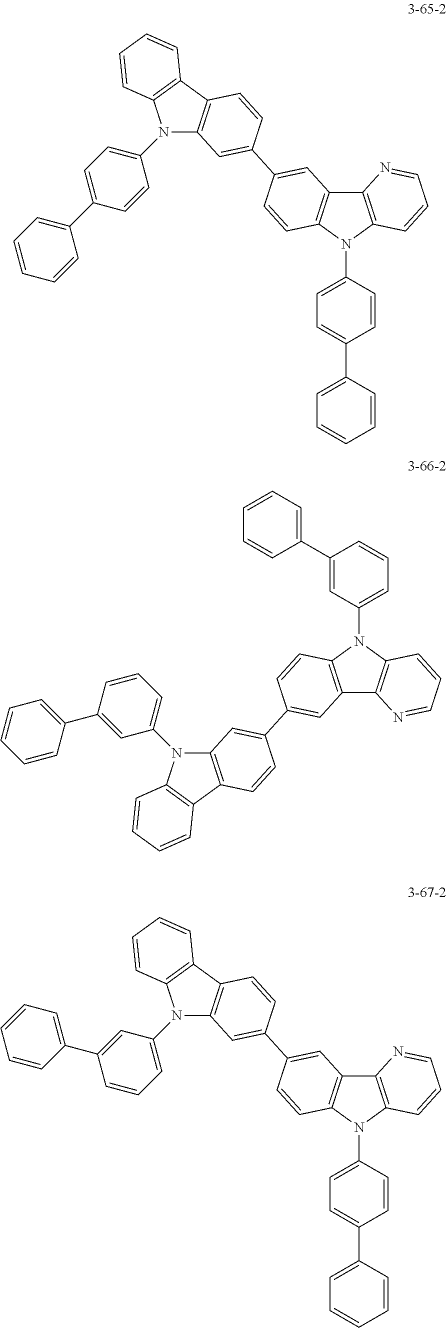 [SMILES: c1ccc(-c2ccc(-n3c4ccccc4c4ccc(-c5ccc6c(c5)c5ncccc5n6-c5ccc(-c6ccccc6)cc5)cc43)cc2)cc1 ; c1ccc(-c2cccc(-n3c4ccccc4c4ccc(-c5ccc6c(c5)c5ncccc5n6-c5cccc(-c6ccccc6)c5)cc43)c2)cc1 ; c1ccc(-c2ccc(-n3c4ccc(-c5ccc6c7ccccc7n(-c7cccc(-c8ccccc8)c7)c6c5)cc4c4ncccc43)cc2)cc1]

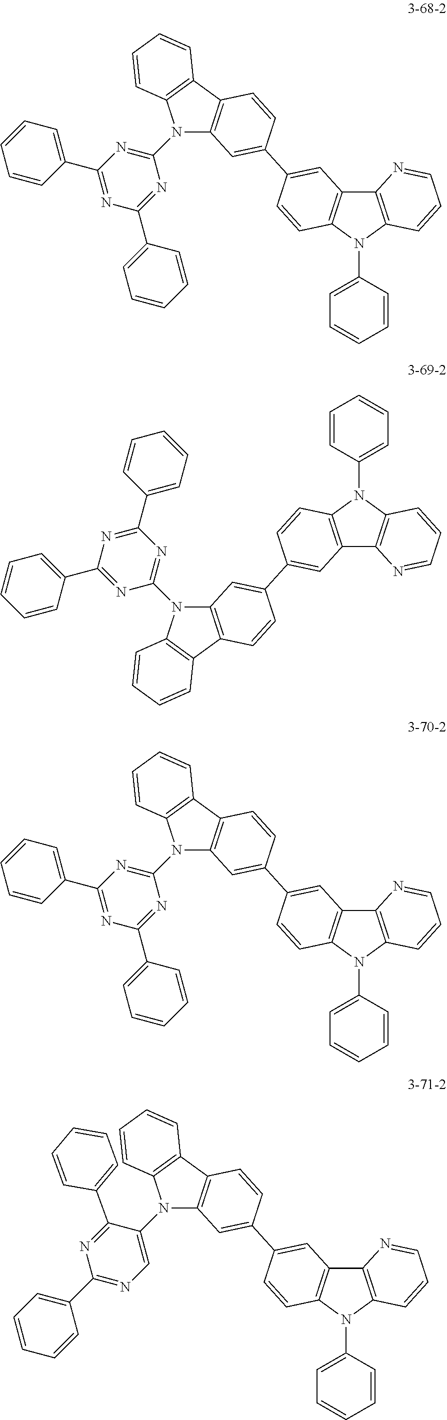 [SMILES: c1ccc(-c2nc(-c3ccccc3)nc(-n3c4ccccc4c4ccc(-c5ccc6c(c5)c5ncccc5n6-c5ccccc5)cc43)n2)cc1 ; c1ccc(-c2nc(-c3ccccc3)nc(-n3c4ccccc4c4ccc(-c5ccc6c(c5)c5ncccc5n6-c5ccccc5)cc43)n2)cc1 ; c1ccc(-c2nc(-c3ccccc3)nc(-n3c4ccccc4c4ccc(-c5ccc6c(c5)c5ncccc5n6-c5ccccc5)cc43)n2)cc1 ; c1ccc(-c2ncc(-n3c4ccccc4c4ccc(-c5ccc6c(c5)c5ncccc5n6-c5ccccc5)cc43)c(-c3ccccc3)n2)cc1]

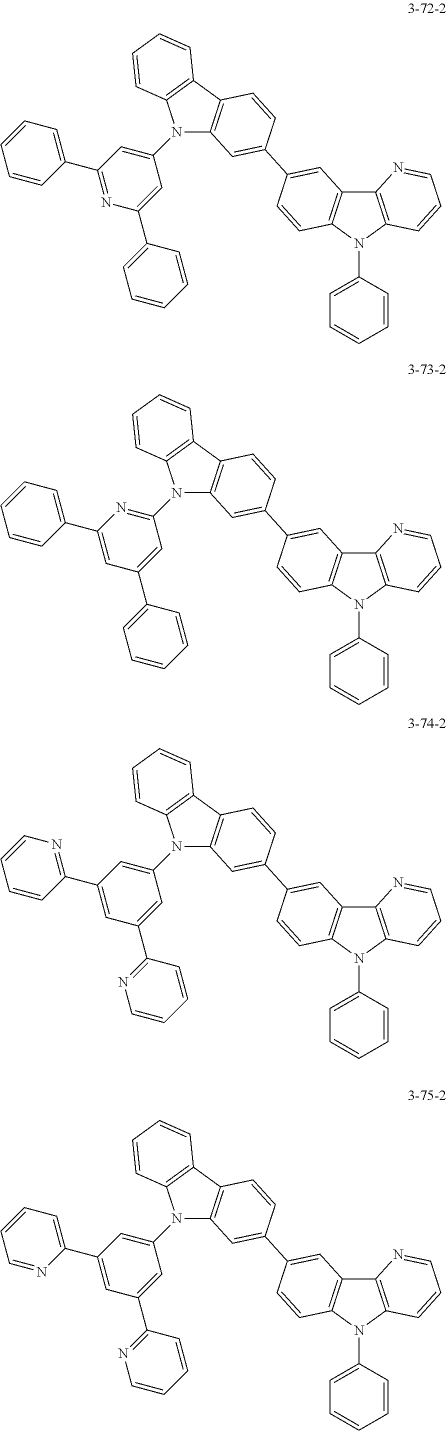 [SMILES: c1ccc(-c2cc(-n3c4ccccc4c4ccc(-c5ccc6c(c5)c5ncccc5n6-c5ccccc5)cc43)cc(-c3ccccc3)n2)cc1 ; c1ccc(-c2cc(-c3ccccc3)nc(-n3c4ccccc4c4ccc(-c5ccc6c(c5)c5ncccc5n6-c5ccccc5)cc43)c2)cc1 ; c1ccc(-n2c3ccc(-c4ccc5c6ccccc6n(-c6cc(-c7ccccn7)cc(-c7ccccn7)c6)c5c4)cc3c3ncccc32)cc1 ; c1ccc(-n2c3ccc(-c4ccc5c6ccccc6n(-c6cc(-c7ccccn7)cc(-c7ccccn7)c6)c5c4)cc3c3ncccc32)cc1]

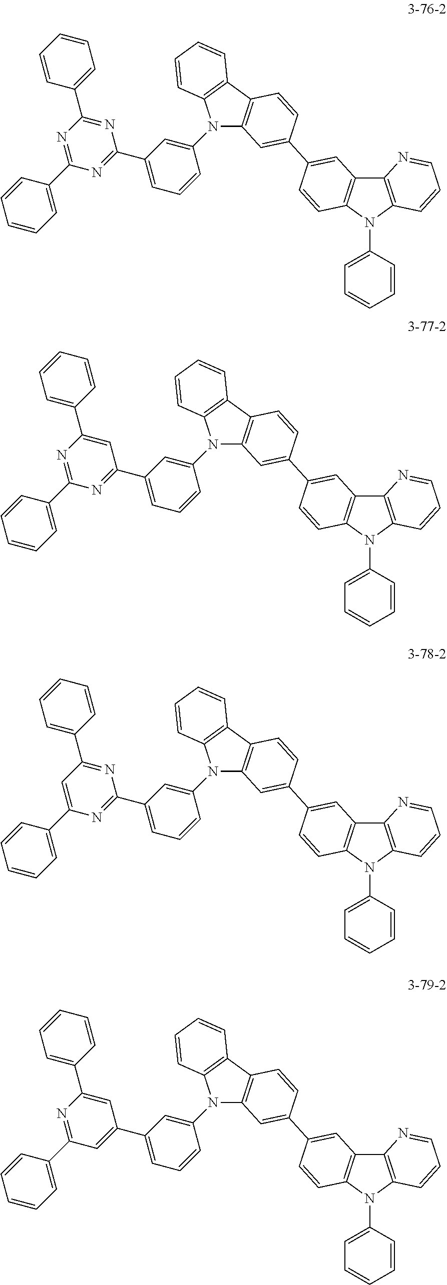 [SMILES: c1ccc(-c2nc(-c3ccccc3)nc(-c3cccc(-n4c5ccccc5c5ccc(-c6ccc7c(c6)c6ncccc6n7-c6ccccc6)cc54)c3)n2)cc1 ; c1ccc(-c2cc(-c3cccc(-n4c5ccccc5c5ccc(-c6ccc7c(c6)c6ncccc6n7-c6ccccc6)cc54)c3)nc(-c3ccccc3)n2)cc1 ; c1ccc(-c2cc(-c3ccccc3)nc(-c3cccc(-n4c5ccccc5c5ccc(-c6ccc7c(c6)c6ncccc6n7-c6ccccc6)cc54)c3)n2)cc1 ; c1ccc(-c2cc(-c3cccc(-n4c5ccccc5c5ccc(-c6ccc7c(c6)c6ncccc6n7-c6ccccc6)cc54)c3)cc(-c3ccccc3)n2)cc1]

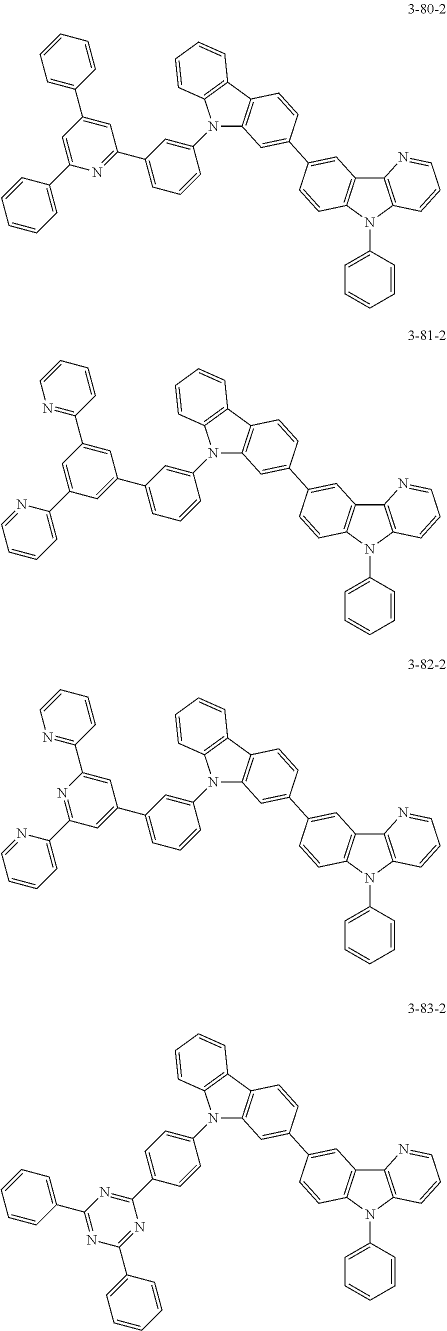 [SMILES: c1ccc(-c2cc(-c3ccccc3)nc(-c3cccc(-n4c5ccccc5c5ccc(-c6ccc7c(c6)c6ncccc6n7-c6ccccc6)cc54)c3)c2)cc1 ; c1ccc(-n2c3ccc(-c4ccc5c6ccccc6n(-c6cccc(-c7cc(-c8ccccn8)cc(-c8ccccn8)c7)c6)c5c4)cc3c3ncccc32)cc1 ; c1ccc(-n2c3ccc(-c4ccc5c6ccccc6n(-c6cccc(-c7cc(-c8ccccn8)nc(-c8ccccn8)c7)c6)c5c4)cc3c3ncccc32)cc1 ; c1ccc(-c2nc(-c3ccccc3)nc(-c3ccc(-n4c5ccccc5c5ccc(-c6ccc7c(c6)c6ncccc6n7-c6ccccc6)cc54)cc3)n2)cc1]

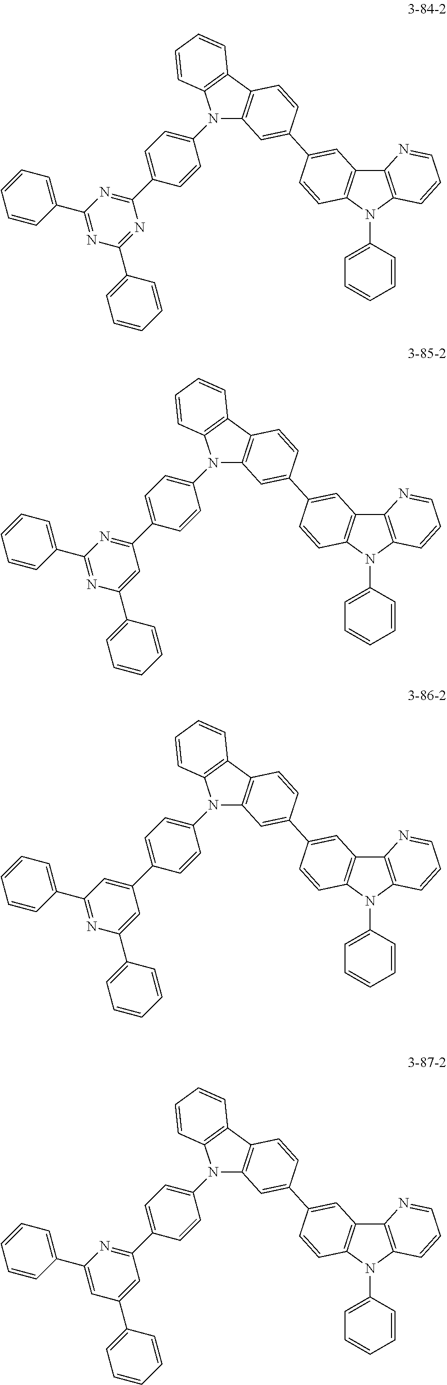 [SMILES: c1ccc(-c2nc(-c3ccccc3)nc(-c3ccc(-n4c5ccccc5c5ccc(-c6ccc7c(c6)c6ncccc6n7-c6ccccc6)cc54)cc3)n2)cc1 ; c1ccc(-c2cc(-c3ccc(-n4c5ccccc5c5ccc(-c6ccc7c(c6)c6ncccc6n7-c6ccccc6)cc54)cc3)nc(-c3ccccc3)n2)cc1 ; c1ccc(-c2cc(-c3ccc(-n4c5ccccc5c5ccc(-c6ccc7c(c6)c6ncccc6n7-c6ccccc6)cc54)cc3)cc(-c3ccccc3)n2)cc1 ; c1ccc(-c2cc(-c3ccccc3)nc(-c3ccc(-n4c5ccccc5c5ccc(-c6ccc7c(c6)c6ncccc6n7-c6ccccc6)cc54)cc3)c2)cc1]

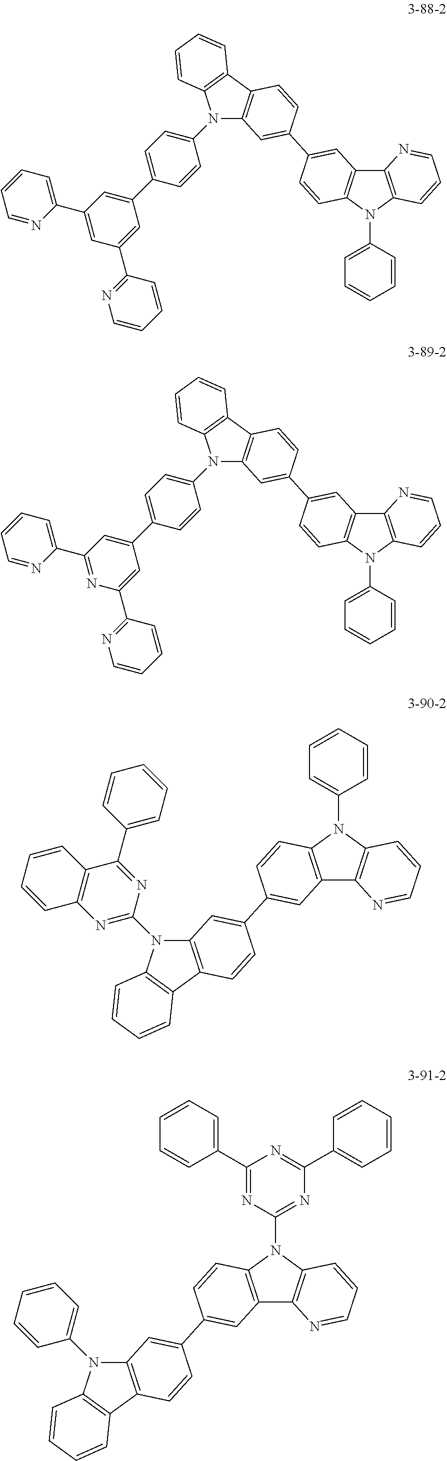 [SMILES: c1ccc(-n2c3ccc(-c4ccc5c6ccccc6n(-c6ccc(-c7cc(-c8ccccn8)cc(-c8ccccn8)c7)cc6)c5c4)cc3c3ncccc32)cc1 ; c1ccc(-n2c3ccc(-c4ccc5c6ccccc6n(-c6ccc(-c7cc(-c8ccccn8)nc(-c8ccccn8)c7)cc6)c5c4)cc3c3ncccc32)cc1 ; c1ccc(-c2nc(-n3c4ccccc4c4ccc(-c5ccc6c(c5)c5ncccc5n6-c5ccccc5)cc43)nc3ccccc23)cc1 ; c1ccc(-c2nc(-c3ccccc3)nc(-n3c4ccc(-c5ccc6c7ccccc7n(-c7ccccc7)c6c5)cc4c4ncccc43)n2)cc1]

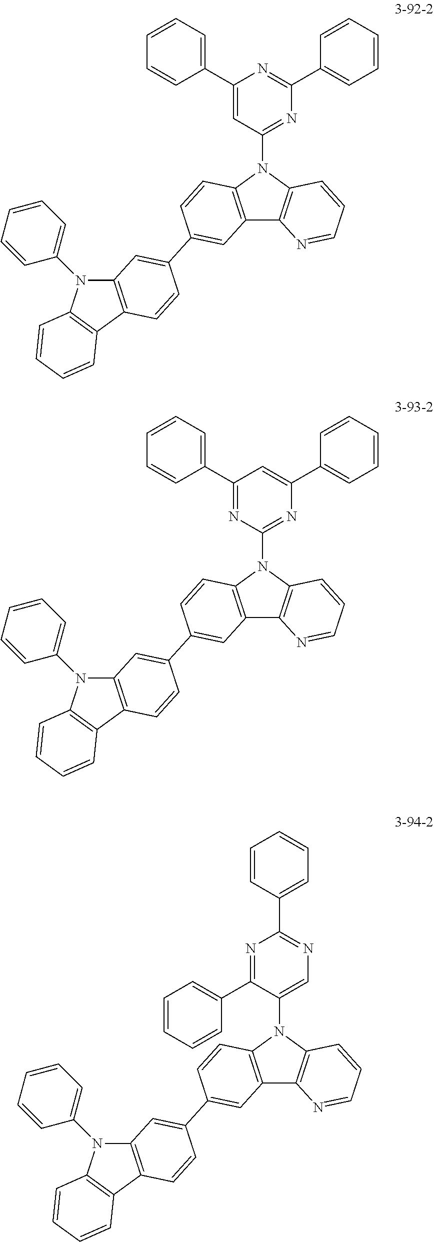 [SMILES: c1ccc(-c2cc(-n3c4ccc(-c5ccc6c7ccccc7n(-c7ccccc7)c6c5)cc4c4ncccc43)nc(-c3ccccc3)n2)cc1 ; c1ccc(-c2cc(-c3ccccc3)nc(-n3c4ccc(-c5ccc6c7ccccc7n(-c7ccccc7)c6c5)cc4c4ncccc43)n2)cc1 ; c1ccc(-c2ncc(-n3c4ccc(-c5ccc6c7ccccc7n(-c7ccccc7)c6c5)cc4c4ncccc43)c(-c3ccccc3)n2)cc1]

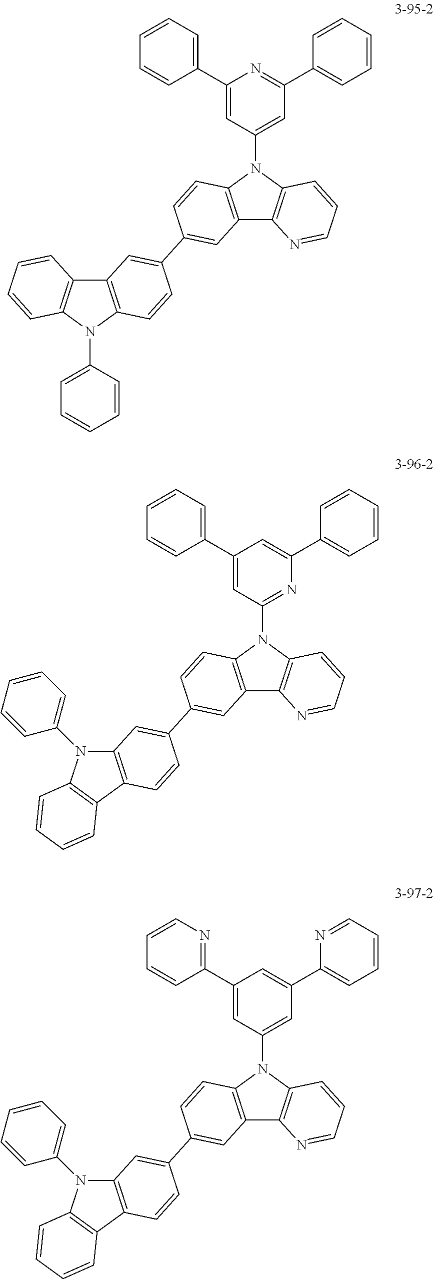 [SMILES: c1ccc(-c2cc(-n3c4ccc(-c5ccc6c(c5)c5ccccc5n6-c5ccccc5)cc4c4ncccc43)cc(-c3ccccc3)n2)cc1 ; c1ccc(-c2cc(-c3ccccc3)nc(-n3c4ccc(-c5ccc6c7ccccc7n(-c7ccccc7)c6c5)cc4c4ncccc43)c2)cc1 ; c1ccc(-n2c3ccccc3c3ccc(-c4ccc5c(c4)c4ncccc4n5-c4cc(-c5ccccn5)cc(-c5ccccn5)c4)cc32)cc1]

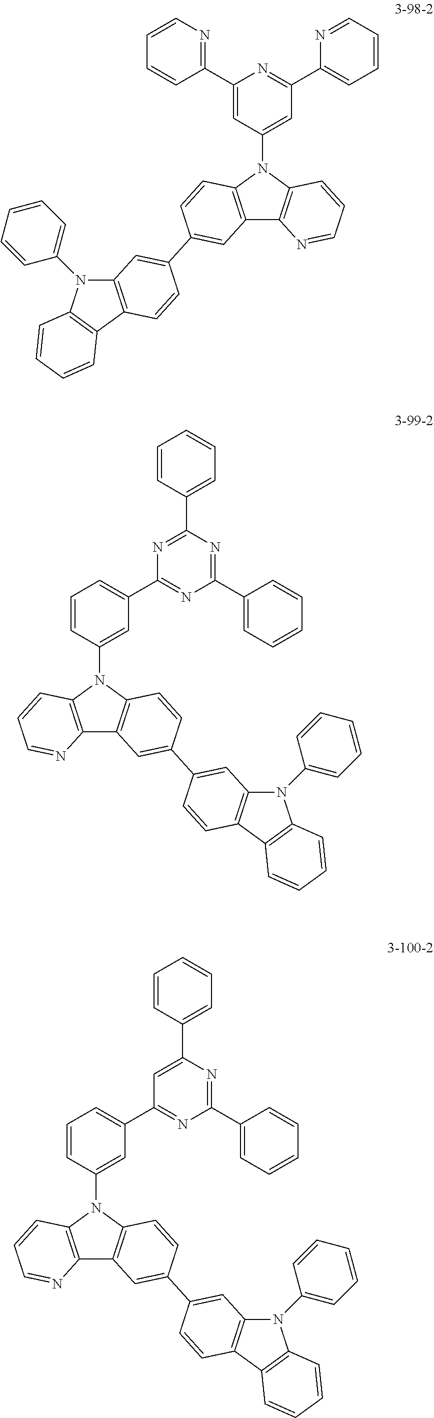 [SMILES: c1ccc(-n2c3ccccc3c3ccc(-c4ccc5c(c4)c4ncccc4n5-c4cc(-c5ccccn5)nc(-c5ccccn5)c4)cc32)cc1 ; c1ccc(-c2nc(-c3ccccc3)nc(-c3cccc(-n4c5ccc(-c6ccc7c8ccccc8n(-c8ccccc8)c7c6)cc5c5ncccc54)c3)n2)cc1 ; c1ccc(-c2cc(-c3cccc(-n4c5ccc(-c6ccc7c8ccccc8n(-c8ccccc8)c7c6)cc5c5ncccc54)c3)nc(-c3ccccc3)n2)cc1]

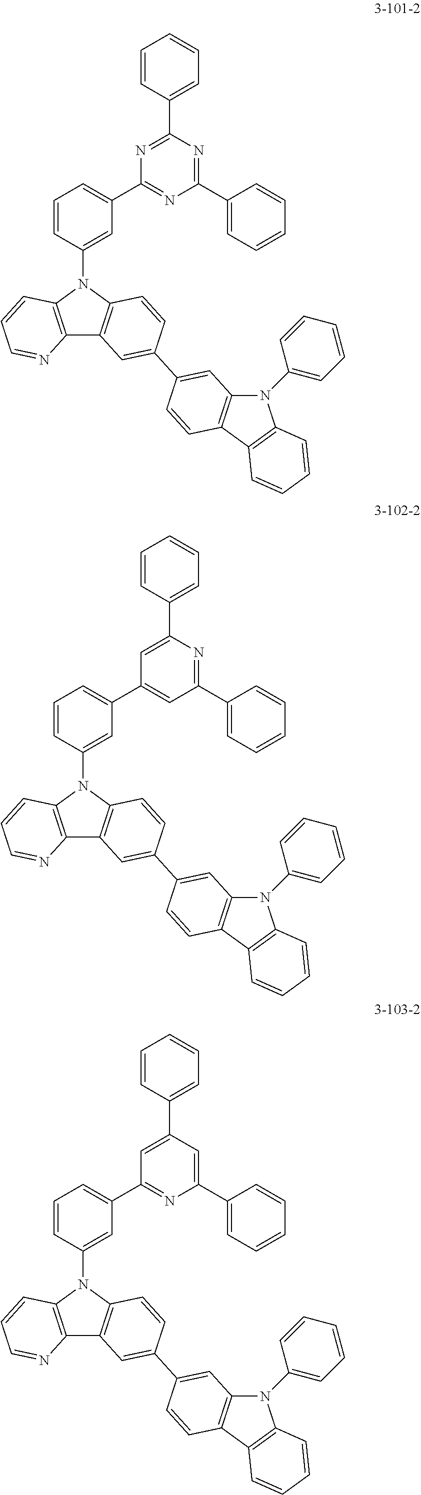 [SMILES: c1ccc(-c2nc(-c3ccccc3)nc(-c3cccc(-n4c5ccc(-c6ccc7c8ccccc8n(-c8ccccc8)c7c6)cc5c5ncccc54)c3)n2)cc1 ; c1ccc(-c2cc(-c3cccc(-n4c5ccc(-c6ccc7c8ccccc8n(-c8ccccc8)c7c6)cc5c5ncccc54)c3)cc(-c3ccccc3)n2)cc1 ; c1ccc(-c2cc(-c3ccccc3)nc(-c3cccc(-n4c5ccc(-c6ccc7c8ccccc8n(-c8ccccc8)c7c6)cc5c5ncccc54)c3)c2)cc1]

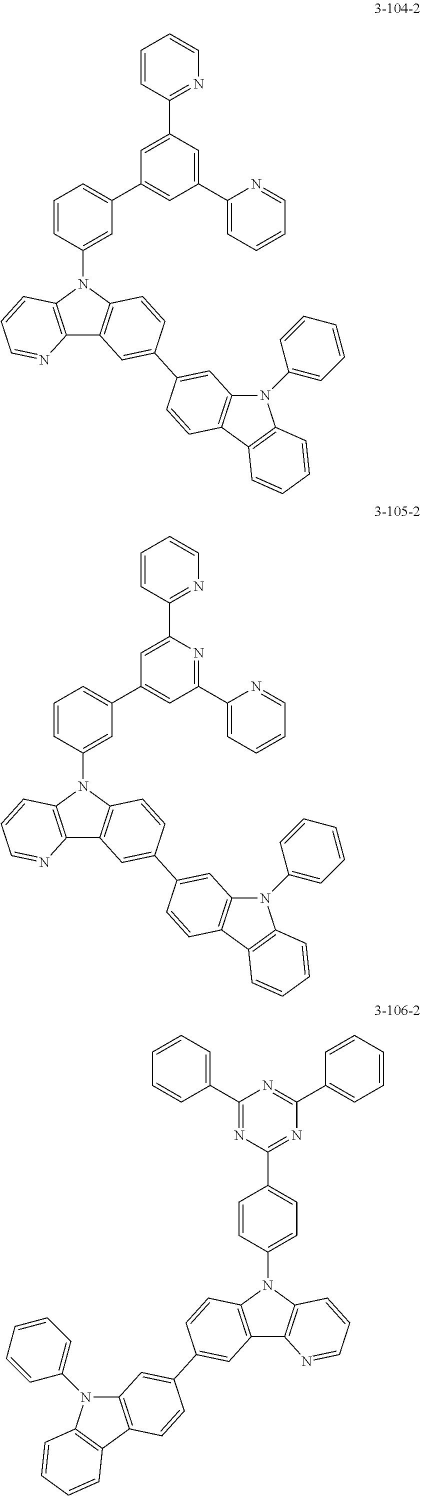 [SMILES: c1ccc(-n2c3ccccc3c3ccc(-c4ccc5c(c4)c4ncccc4n5-c4cccc(-c5cc(-c6ccccn6)cc(-c6ccccn6)c5)c4)cc32)cc1 ; c1ccc(-n2c3ccccc3c3ccc(-c4ccc5c(c4)c4ncccc4n5-c4cccc(-c5cc(-c6ccccn6)nc(-c6ccccn6)c5)c4)cc32)cc1 ; c1ccc(-c2nc(-c3ccccc3)nc(-c3ccc(-n4c5ccc(-c6ccc7c8ccccc8n(-c8ccccc8)c7c6)cc5c5ncccc54)cc3)n2)cc1]

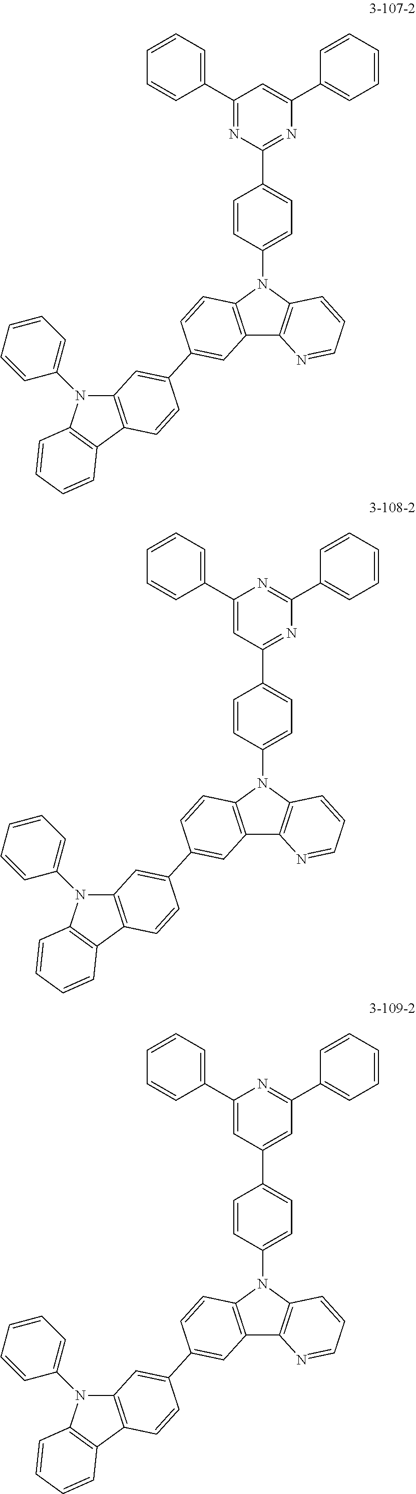 [SMILES: c1ccc(-c2cc(-c3ccccc3)nc(-c3ccc(-n4c5ccc(-c6ccc7c8ccccc8n(-c8ccccc8)c7c6)cc5c5ncccc54)cc3)n2)cc1 ; c1ccc(-c2cc(-c3ccc(-n4c5ccc(-c6ccc7c8ccccc8n(-c8ccccc8)c7c6)cc5c5ncccc54)cc3)nc(-c3ccccc3)n2)cc1 ; c1ccc(-c2cc(-c3ccc(-n4c5ccc(-c6ccc7c8ccccc8n(-c8ccccc8)c7c6)cc5c5ncccc54)cc3)cc(-c3ccccc3)n2)cc1]

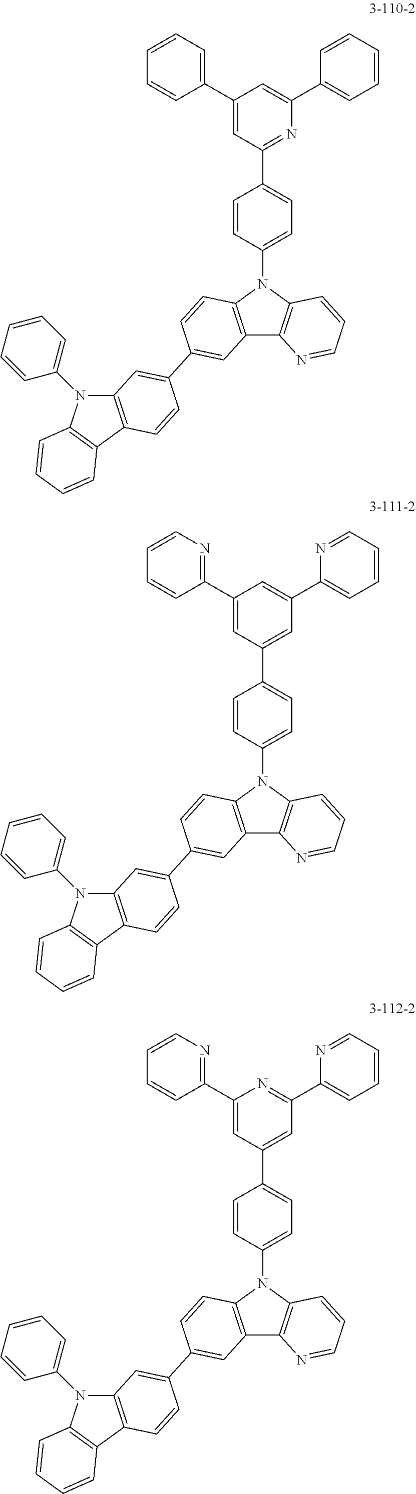 [SMILES: c1ccc(-c2cc(-c3ccccc3)nc(-c3ccc(-n4c5ccc(-c6ccc7c8ccccc8n(-c8ccccc8)c7c6)cc5c5ncccc54)cc3)c2)cc1 ; c1ccc(-n2c3ccccc3c3ccc(-c4ccc5c(c4)c4ncccc4n5-c4ccc(-c5cc(-c6ccccn6)cc(-c6ccccn6)c5)cc4)cc32)cc1 ; c1ccc(-n2c3ccccc3c3ccc(-c4ccc5c(c4)c4ncccc4n5-c4ccc(-c5cc(-c6ccccn6)nc(-c6ccccn6)c5)cc4)cc32)cc1]

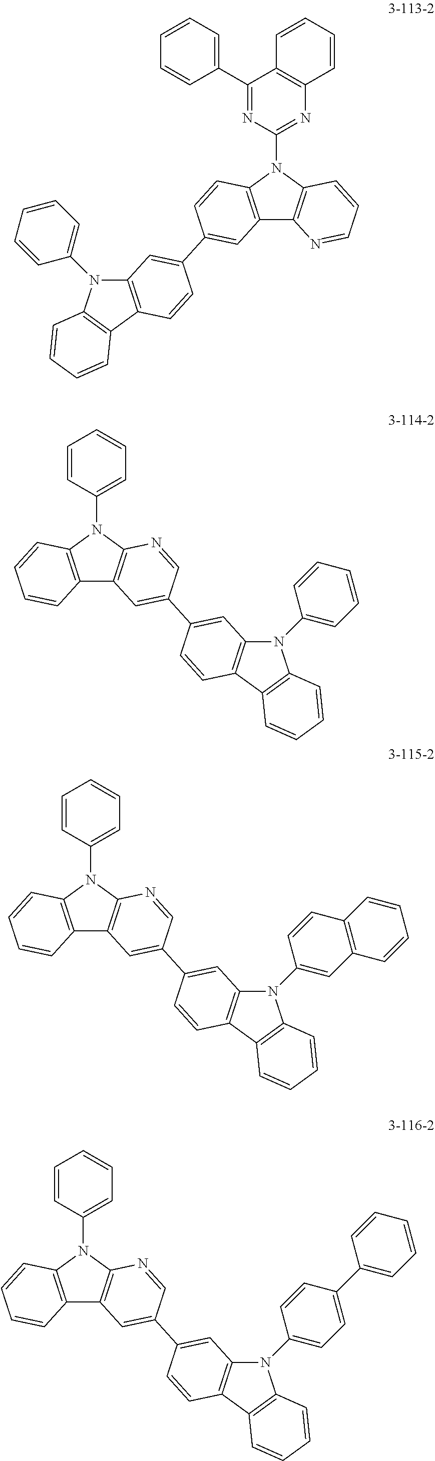 [SMILES: c1ccc(-c2nc(-n3c4ccc(-c5ccc6c7ccccc7n(-c7ccccc7)c6c5)cc4c4ncccc43)nc3ccccc23)cc1 ; c1ccc(-n2c3ccccc3c3ccc(-c4cnc5c(c4)c4ccccc4n5-c4ccccc4)cc32)cc1 ; c1ccc(-n2c3ccccc3c3cc(-c4ccc5c6ccccc6n(-c6ccc7ccccc7c6)c5c4)cnc32)cc1 ; c1ccc(-c2ccc(-n3c4ccccc4c4ccc(-c5cnc6c(c5)c5ccccc5n6-c5ccccc5)cc43)cc2)cc1]

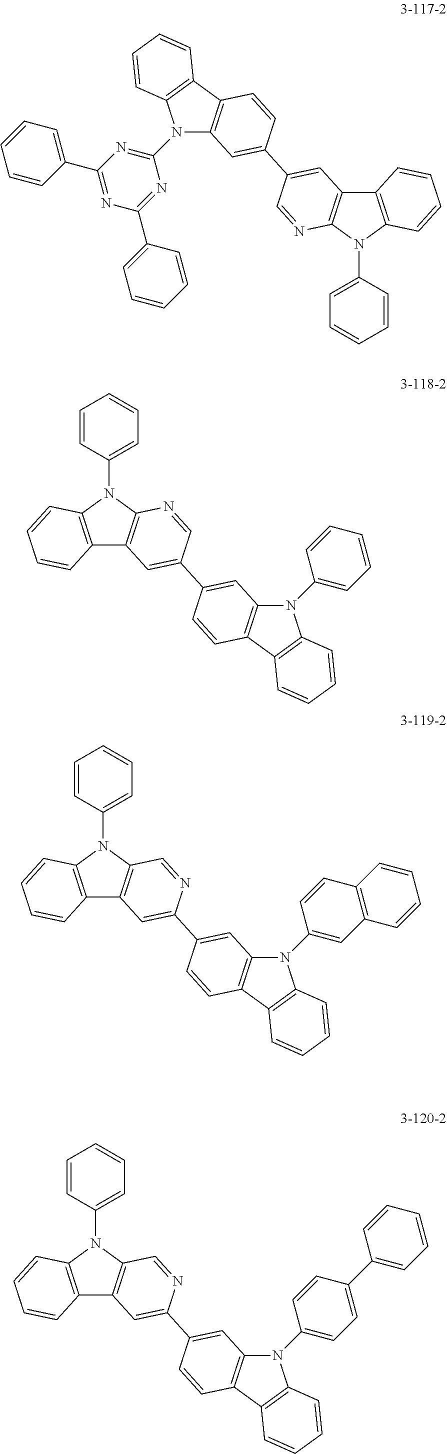 [SMILES: c1ccc(-c2nc(-c3ccccc3)nc(-n3c4ccccc4c4ccc(-c5cnc6c(c5)c5ccccc5n6-c5ccccc5)cc43)n2)cc1 ; c1ccc(-n2c3ccccc3c3ccc(-c4cnc5c(c4)c4ccccc4n5-c4ccccc4)cc32)cc1 ; c1ccc(-n2c3ccccc3c3cc(-c4ccc5c6ccccc6n(-c6ccc7ccccc7c6)c5c4)ncc32)cc1 ; c1ccc(-c2ccc(-n3c4ccccc4c4ccc(-c5cc6c7ccccc7n(-c7ccccc7)c6cn5)cc43)cc2)cc1]

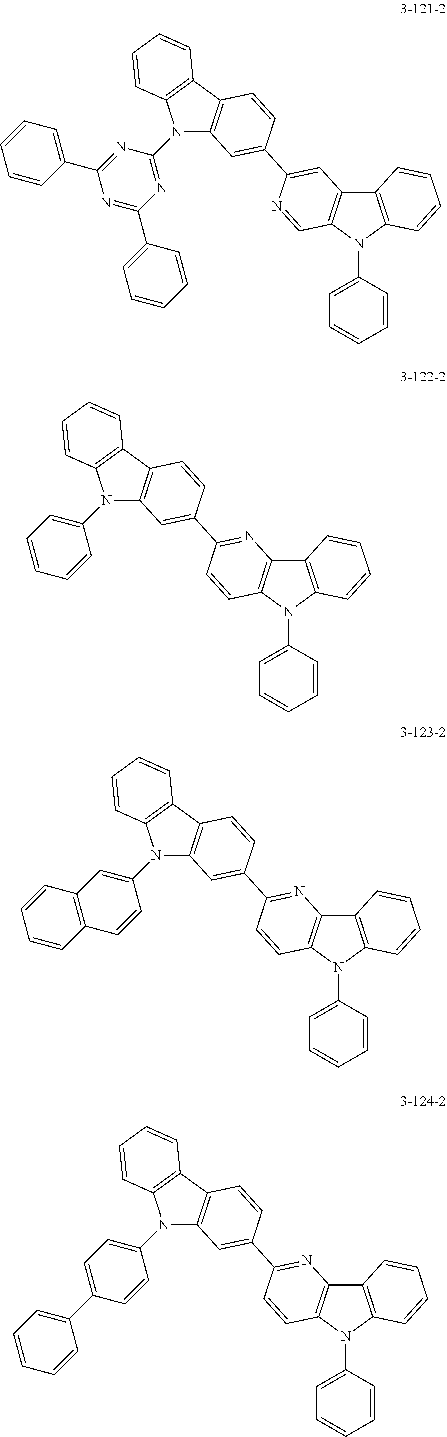 [SMILES: c1ccc(-c2nc(-c3ccccc3)nc(-n3c4ccccc4c4ccc(-c5cc6c7ccccc7n(-c7ccccc7)c6cn5)cc43)n2)cc1 ; c1ccc(-n2c3ccccc3c3ccc(-c4ccc5c(n4)c4ccccc4n5-c4ccccc4)cc32)cc1 ; c1ccc(-n2c3ccccc3c3nc(-c4ccc5c6ccccc6n(-c6ccc7ccccc7c6)c5c4)ccc32)cc1 ; c1ccc(-c2ccc(-n3c4ccccc4c4ccc(-c5ccc6c(n5)c5ccccc5n6-c5ccccc5)cc43)cc2)cc1]

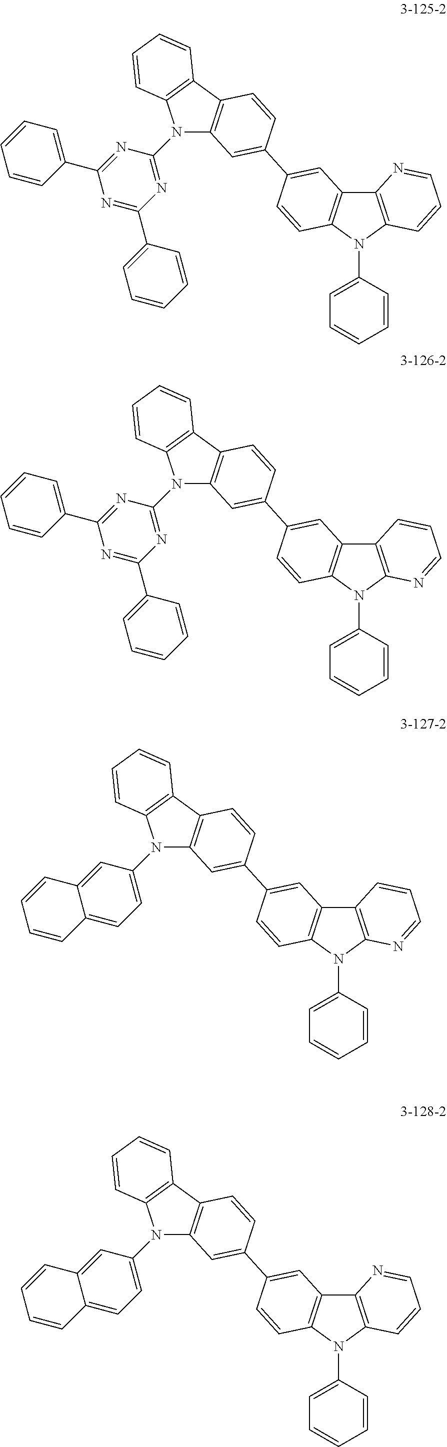 [SMILES: c1ccc(-c2nc(-c3ccccc3)nc(-n3c4ccccc4c4ccc(-c5ccc6c(c5)c5ncccc5n6-c5ccccc5)cc43)n2)cc1 ; c1ccc(-c2nc(-c3ccccc3)nc(-n3c4ccccc4c4ccc(-c5ccc6c(c5)c5cccnc5n6-c5ccccc5)cc43)n2)cc1 ; c1ccc(-n2c3ccc(-c4ccc5c6ccccc6n(-c6ccc7ccccc7c6)c5c4)cc3c3cccnc32)cc1 ; c1ccc(-n2c3ccc(-c4ccc5c6ccccc6n(-c6ccc7ccccc7c6)c5c4)cc3c3ncccc32)cc1]

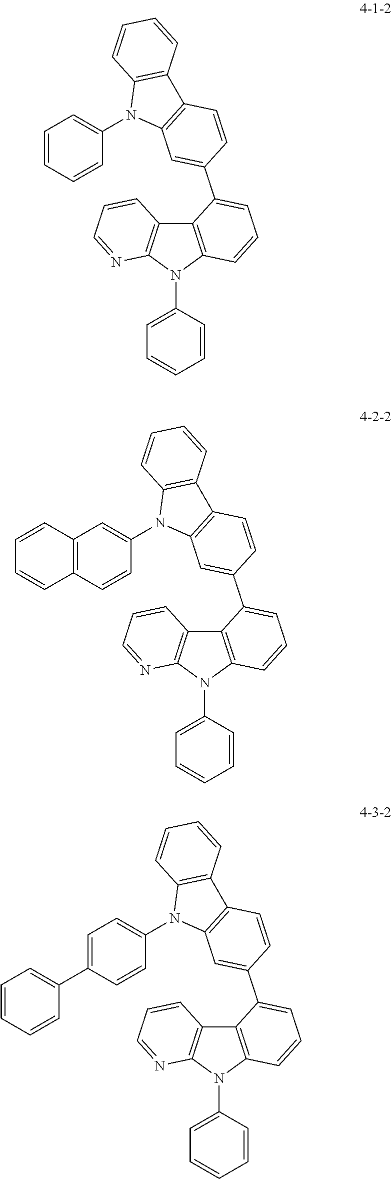

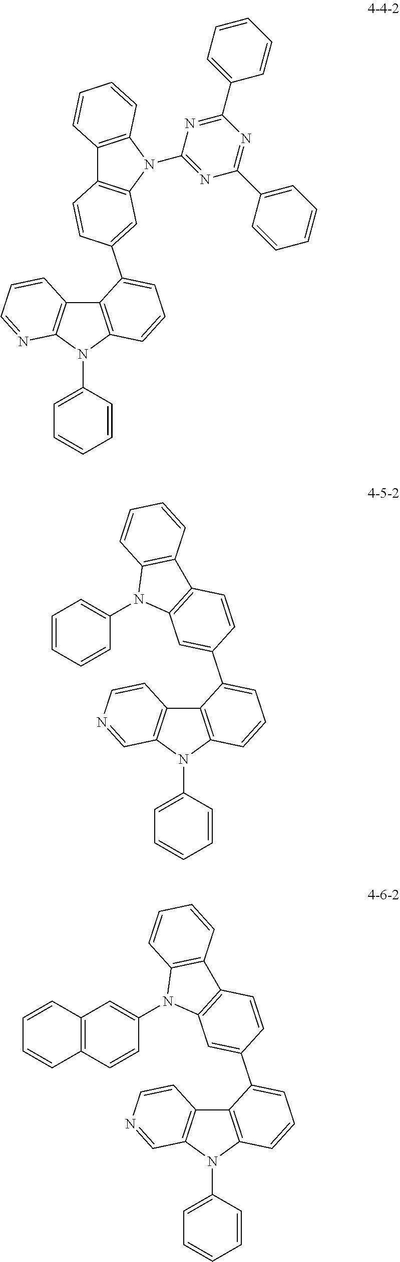

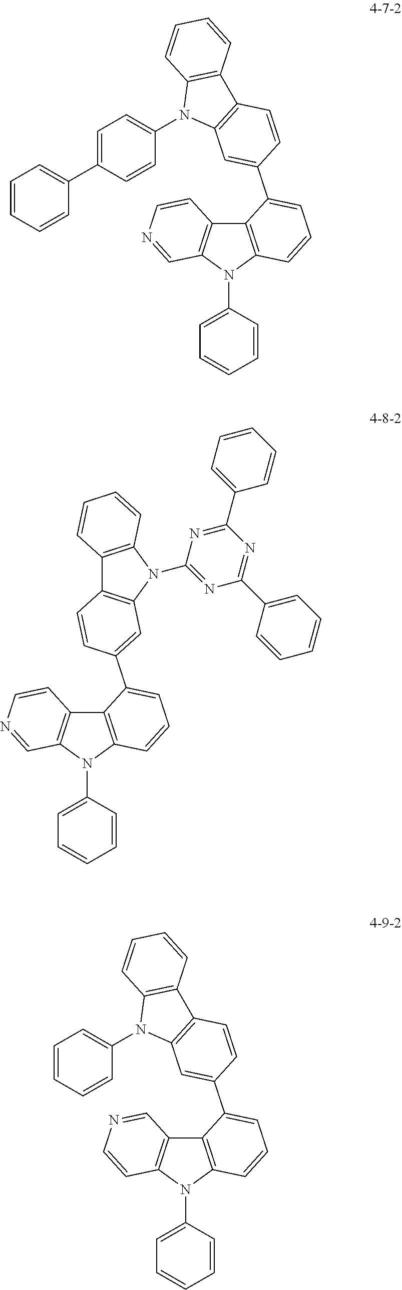 [SMILES: c1ccc(-c2ccc(-n3c4ccccc4c4ccc(-c5cccc6c5c5ccncc5n6-c5ccccc5)cc43)cc2)cc1 ; c1ccc(-c2nc(-c3ccccc3)nc(-n3c4ccccc4c4ccc(-c5cccc6c5c5ccncc5n6-c5ccccc5)cc43)n2)cc1 ; c1ccc(-n2c3ccccc3c3ccc(-c4cccc5c4c4cnccc4n5-c4ccccc4)cc32)cc1]

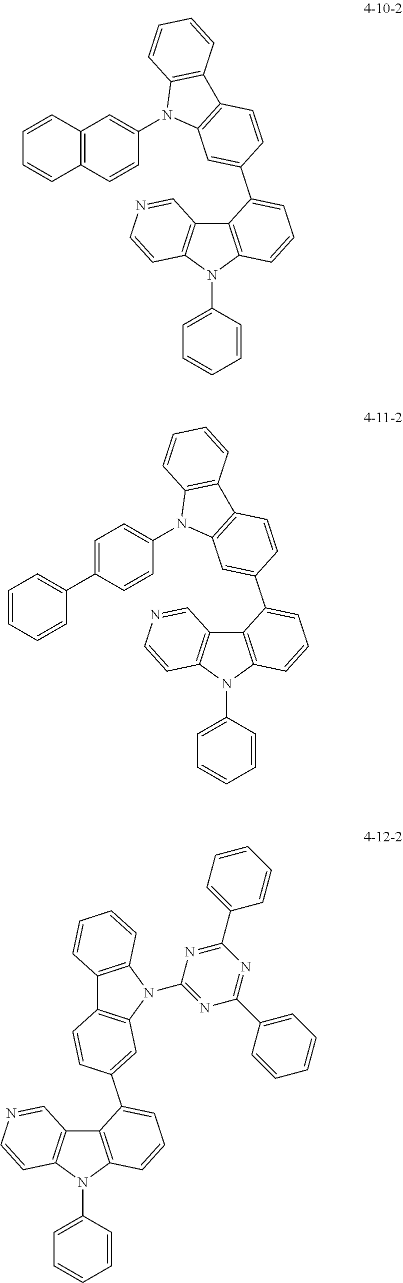 [SMILES: c1ccc(-n2c3ccncc3c3c(-c4ccc5c6ccccc6n(-c6ccc7ccccc7c6)c5c4)cccc32)cc1 ; c1ccc(-c2ccc(-n3c4ccccc4c4ccc(-c5cccc6c5c5cnccc5n6-c5ccccc5)cc43)cc2)cc1 ; c1ccc(-c2nc(-c3ccccc3)nc(-n3c4ccccc4c4ccc(-c5cccc6c5c5cnccc5n6-c5ccccc5)cc43)n2)cc1]

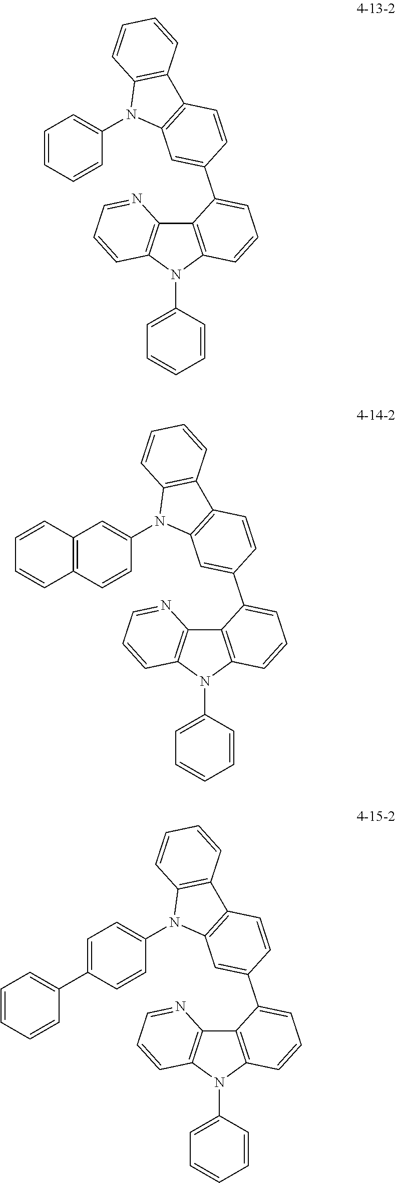

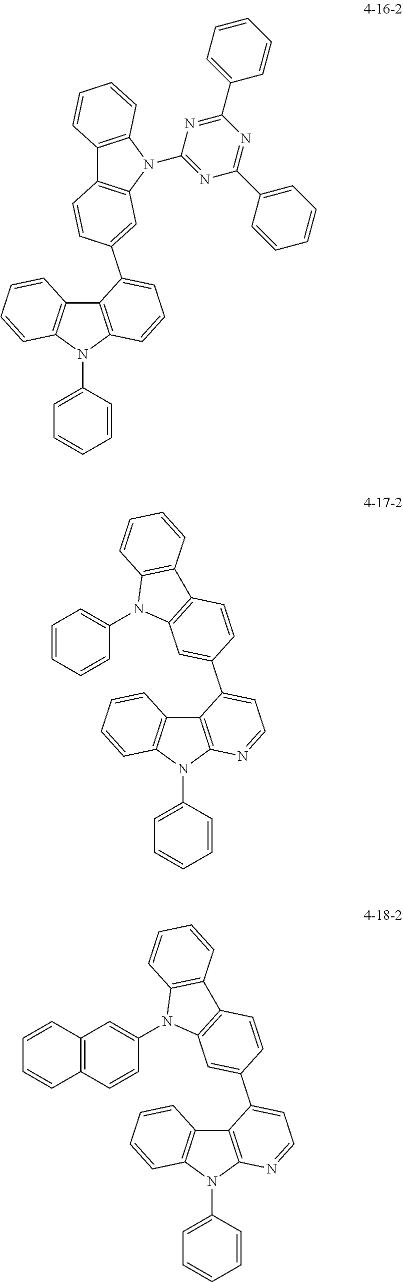 [SMILES: c1ccc(-c2nc(-c3ccccc3)nc(-n3c4ccccc4c4ccc(-c5cccc6c5c5ccccc5n6-c5ccccc5)cc43)n2)cc1 ; c1ccc(-n2c3ccccc3c3ccc(-c4ccnc5c4c4ccccc4n5-c4ccccc4)cc32)cc1 ; c1ccc(-n2c3ccccc3c3c(-c4ccc5c6ccccc6n(-c6ccc7ccccc7c6)c5c4)ccnc32)cc1]

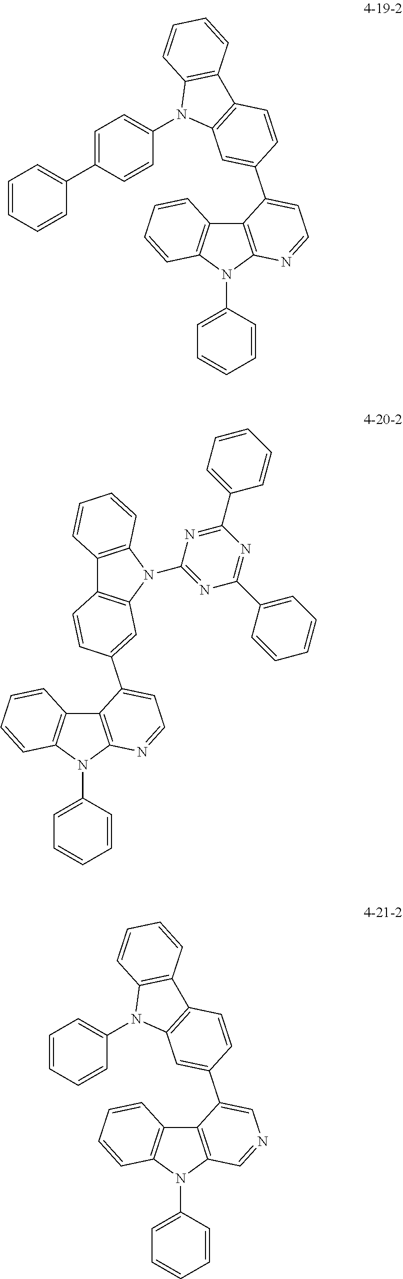 [SMILES: c1ccc(-c2ccc(-n3c4ccccc4c4ccc(-c5ccnc6c5c5ccccc5n6-c5ccccc5)cc43)cc2)cc1 ; c1ccc(-c2nc(-c3ccccc3)nc(-n3c4ccccc4c4ccc(-c5ccnc6c5c5ccccc5n6-c5ccccc5)cc43)n2)cc1 ; c1ccc(-n2c3ccccc3c3ccc(-c4cncc5c4c4ccccc4n5-c4ccccc4)cc32)cc1]

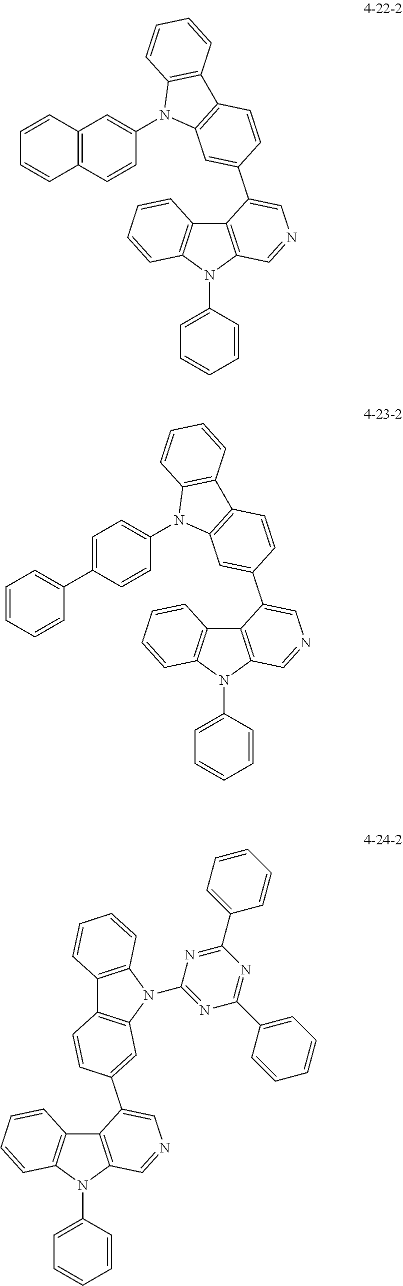 [SMILES: c1ccc(-n2c3ccccc3c3c(-c4ccc5c6ccccc6n(-c6ccc7ccccc7c6)c5c4)cncc32)cc1 ; c1ccc(-c2ccc(-n3c4ccccc4c4ccc(-c5cncc6c5c5ccccc5n6-c5ccccc5)cc43)cc2)cc1 ; c1ccc(-c2nc(-c3ccccc3)nc(-n3c4ccccc4c4ccc(-c5cncc6c5c5ccccc5n6-c5ccccc5)cc43)n2)cc1]

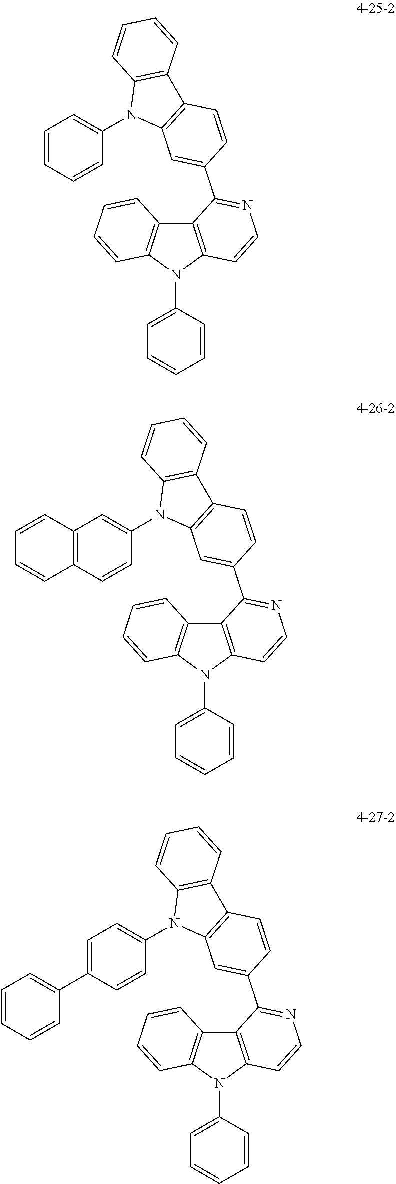

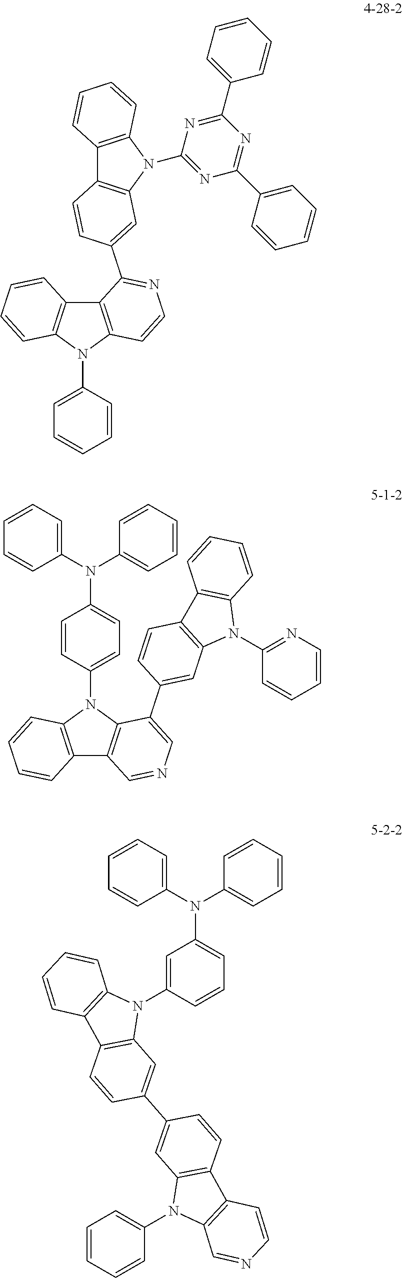 [SMILES: c1ccc(-c2nc(-c3ccccc3)nc(-n3c4ccccc4c4ccc(-c5nccc6c5c5ccccc5n6-c5ccccc5)cc43)n2)cc1 ; c1ccc(N(c2ccccc2)c2ccc(-n3c4ccccc4c4cncc(-c5ccc6c7ccccc7n(-c7ccccn7)c6c5)c43)cc2)cc1 ; c1ccc(N(c2ccccc2)c2cccc(-n3c4ccccc4c4ccc(-c5ccc6c7ccncc7n(-c7ccccc7)c6c5)cc43)c2)cc1]

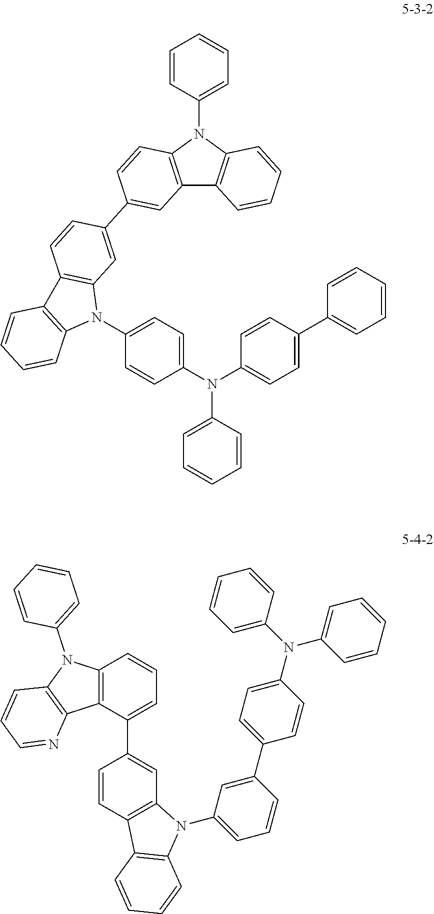 [SMILES: c1ccc(-c2ccc(N(c3ccccc3)c3ccc(-n4c5ccccc5c5ccc(-c6ccc7c(c6)c6ccccc6n7-c6ccccc6)cc54)cc3)cc2)cc1 ; c1ccc(N(c2ccccc2)c2ccc(-c3cccc(-n4c5ccccc5c5ccc(-c6cccc7c6c6ncccc6n7-c6ccccc6)cc54)c3)cc2)cc1]

In another embodiment, the present invention provides a compound for an organic electronic element, represented by Formula 2-1.

In still another embodiment, the present invention provides an organic electronic element containing the compound represented by Formula 2-1.

Here, the organic electronic element may include: a first electrode; a second electrode; and an organic material layer positioned between the first electrode and the second electrode, wherein the organic material layer may contain a compound represented by Formula 2-1, and the compound represented by Formula 2-1 may be contained in at least one of a hole injection layer, a hole transport layer, an auxiliary light emitting layer, a light emitting layer, an electron transport layer, and an electron injection layer for an organic material layer. Especially, the compound represented by Formula 2-1 may be contained in the light emitting layer.

That is, the compound represented by Formula 2-1 may be used as a material for a hole injection layer, a hole transport layer, an auxiliary light emitting layer, a light emitting layer, an electron transport layer, or an electron injection layer. Especially, the compound represented by Formula 2-1 may be used as a material for the light emitting layer. The present invention provides, specifically, an organic electronic element including an organic material layer containing one of the compounds represented by Formulas 2-2 to 2-13, and more specifically, an organic electronic element including an organic material layer containing the compound represented by an individual formula (1-1-2 to 1-28-2, 2-1-2 to 2-128-2, 3-1-2 to 3-128-2, 4-1-2 to 4-28-2, and 5-1-2 to 5-4-2).

In still another embodiment, the present invention provides an organic electronic element, in which the compound is contained alone, two or more different types of the compounds are contained as a combination, or the compound is contained together with other compounds as a combination of two or more in at least one of the hole injection layer, the hole transport layer, the auxiliary light emitting layer, the light emitting layer, the electron transport layer, and the electron injection layer of the organic material layer. In other words, the compounds corresponding to Formulas 2-1 to 2-13 may be contained alone, a mixture of two or more kinds of compounds of Formulas 2-1 to 2-13 may be contained, or a mixture of the compound of the claims and a compound not corresponding to the present invention may be contained in each of the layers. Here, the compounds that do not correspond to the present invention may be a single compound or two or more kinds of compounds. Here, when the compound is contained together with other compounds as a combination of two or more kinds of compounds, another compound may be a compound that is already known for each organic material layer, or a compound to be developed in the future. Here, the compounds contained in the organic material layer may be composed of only the same kind of compounds, or a mixture of two or more kinds of different compounds represented by formula 2-1.

In still another embodiment of the present invention, the present invention provides an organic electronic element further including a light efficiency improvement layer, which is formed on at least one of one side of one surface of the first electrode, which is opposite to the organic material layer and one side of one surface of the second electrode, which is opposite to the organic material layer.

Hereinafter, synthesis examples of the compound represented by Formula 2-1 and manufacturing examples of the organic electronic element according to the present invention will be described in detail by way of examples. However, the following examples are only for illustrative purposes and are not intended to limit the scope of the invention.

### SYNTHESIS EXAMPLES

The product represented by Formula 2-1 according to the present invention are prepared by reaction of Sub 2-1 and Sub 2-2 as in Reaction Scheme 2-1 below, but are not limited thereto.

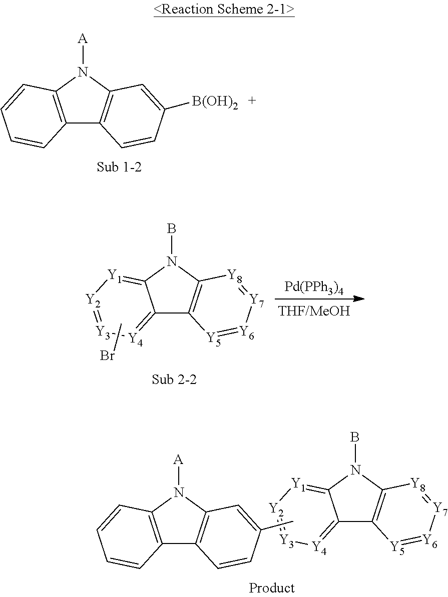

### I. Synthesis Example of Sub 2-1

Sub 2-1 in Reaction Scheme 2-1 may be synthesized via the reaction pathway of Reaction Scheme 2-2 below, but is not limited thereto.

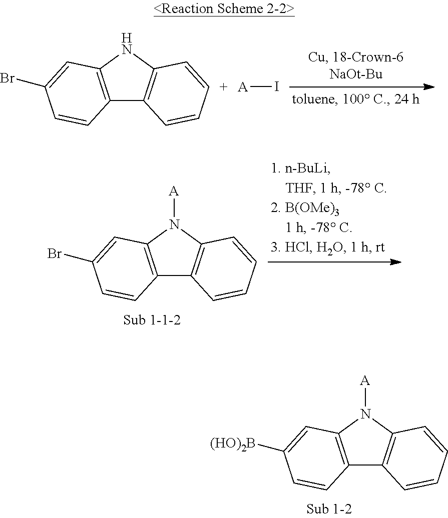

### Synthesis Sub 1-1-2

After bromo-9H-carbazole (203 mmol) and an iodo compound (240 mmol) were mixed with 800 mL of toluene, Cu (764 mg, 12 mmol), 18-Crown-6 (6.3 g, 24 mmol), and NaOt-Bu (57.6 g, 600 mmol) were added thereto, and the mixture was stirred under reflux at 100□ for 24 h. After extraction with ether and water, the organic layer was dried over MgSO₄ and concentrated, and then the generated organic material was subjected to silica gel column chromatography and recrystallization to give an intermediate.

### Synthesis of Sub 1-1(1)-2

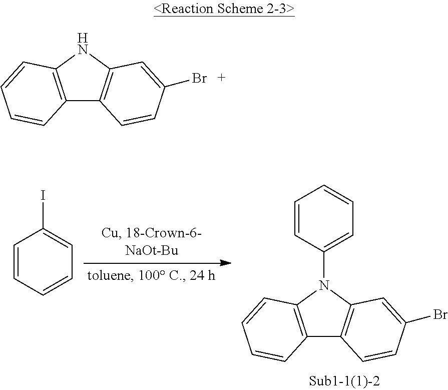 [SMILES: Brc1ccc2c(c1)[nH]c1ccccc12 ; Ic1ccccc1 ; Brc1ccc2c3ccccc3n(-c3ccccc3)c2c1]

After bromo-9H-carbazole (50 g, 203 mmol) and iodobenzene (49 g, 240 mmol) were mixed with 800 mL of toluene, Cu (764 mg, 12 mmol), 18-Crown-6 (6.3 g, 24 mmol), and NaOt-Bu (57.6 g, 600 mmol) were added thereto, and the mixture was stirred under reflux at 100□ for 24 h. After extraction with ether and water, the organic layer was dried over MgSO₄ and concentrated, and then the generated organic material was subjected to silica gel column chromatography and recrystallization to give 37.9 g of Sub 1-1(1)-2 (yield: 58%).

Examples of Sub 1-1-2 are as follows, but are limited thereto, and FD-MS values thereof are shown in table 2-1

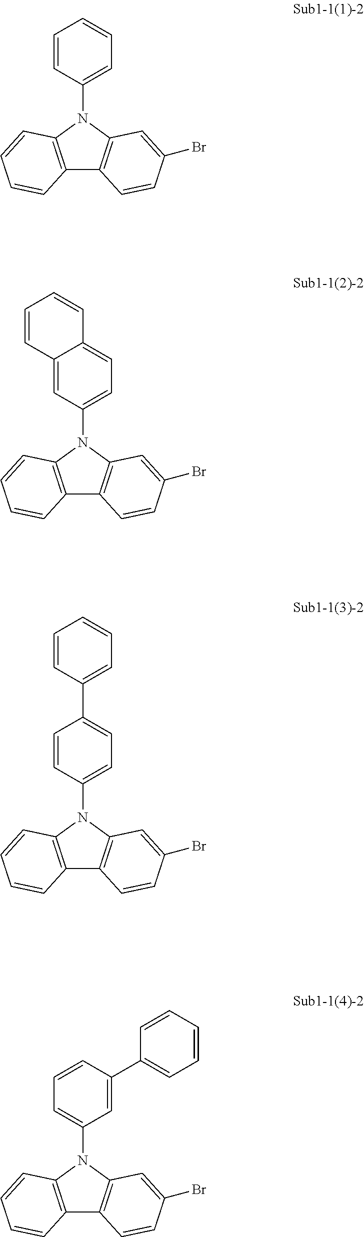 [SMILES: Brc1ccc2c3ccccc3n(-c3ccccc3)c2c1 ; Brc1ccc2c3ccccc3n(-c3ccc4ccccc4c3)c2c1 ; Brc1ccc2c3ccccc3n(-c3ccc(-c4ccccc4)cc3)c2c1 ; Brc1ccc2c3ccccc3n(-c3cccc(-c4ccccc4)c3)c2c1]

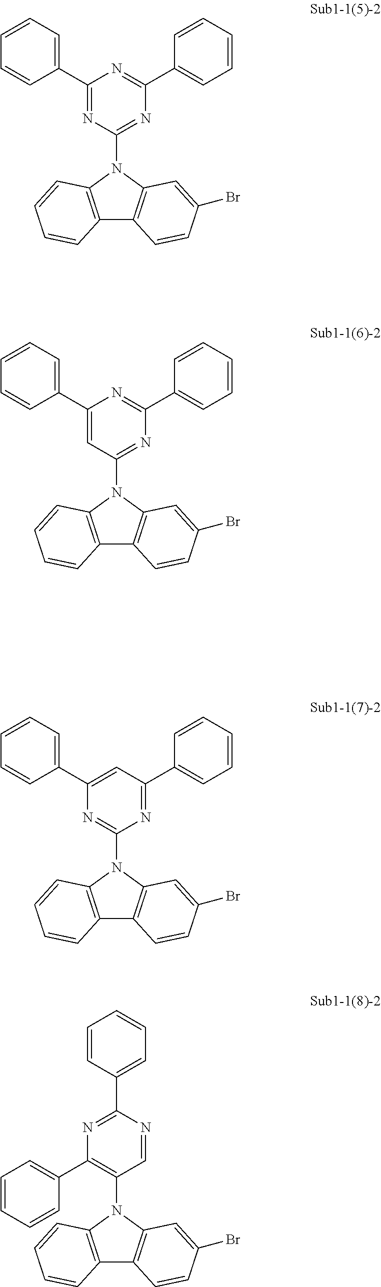 [SMILES: Brc1ccc2c3ccccc3n(-c3nc(-c4ccccc4)nc(-c4ccccc4)n3)c2c1 ; Brc1ccc2c3ccccc3n(-c3cc(-c4ccccc4)nc(-c4ccccc4)n3)c2c1 ; Brc1ccc2c3ccccc3n(-c3nc(-c4ccccc4)cc(-c4ccccc4)n3)c2c1 ; Brc1ccc2c3ccccc3n(-c3cnc(-c4ccccc4)nc3-c3ccccc3)c2c1]

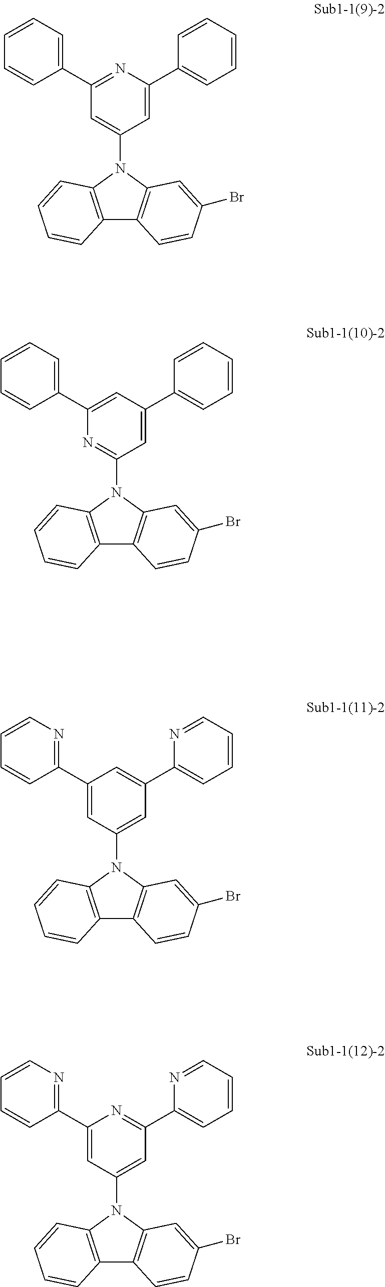 [SMILES: Brc1ccc2c3ccccc3n(-c3cc(-c4ccccc4)nc(-c4ccccc4)c3)c2c1 ; Brc1ccc2c3ccccc3n(-c3cc(-c4ccccc4)cc(-c4ccccc4)n3)c2c1 ; Brc1ccc2c3ccccc3n(-c3cc(-c4ccccn4)cc(-c4ccccn4)c3)c2c1 ; Brc1ccc2c3ccccc3n(-c3cc(-c4ccccn4)nc(-c4ccccn4)c3)c2c1]

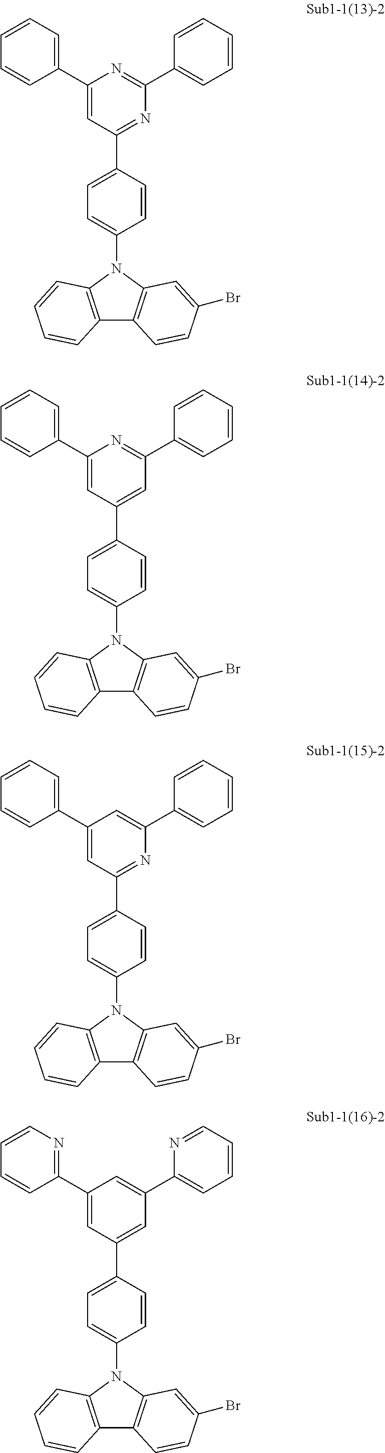 [SMILES: Brc1ccc2c3ccccc3n(-c3ccc(-c4cc(-c5ccccc5)nc(-c5ccccc5)n4)cc3)c2c1 ; Brc1ccc2c3ccccc3n(-c3ccc(-c4cc(-c5ccccc5)nc(-c5ccccc5)c4)cc3)c2c1 ; Brc1ccc2c3ccccc3n(-c3ccc(-c4cc(-c5ccccc5)cc(-c5ccccc5)n4)cc3)c2c1 ; Brc1ccc2c3ccccc3n(-c3ccc(-c4cc(-c5ccccn5)cc(-c5ccccn5)c4)cc3)c2c1]

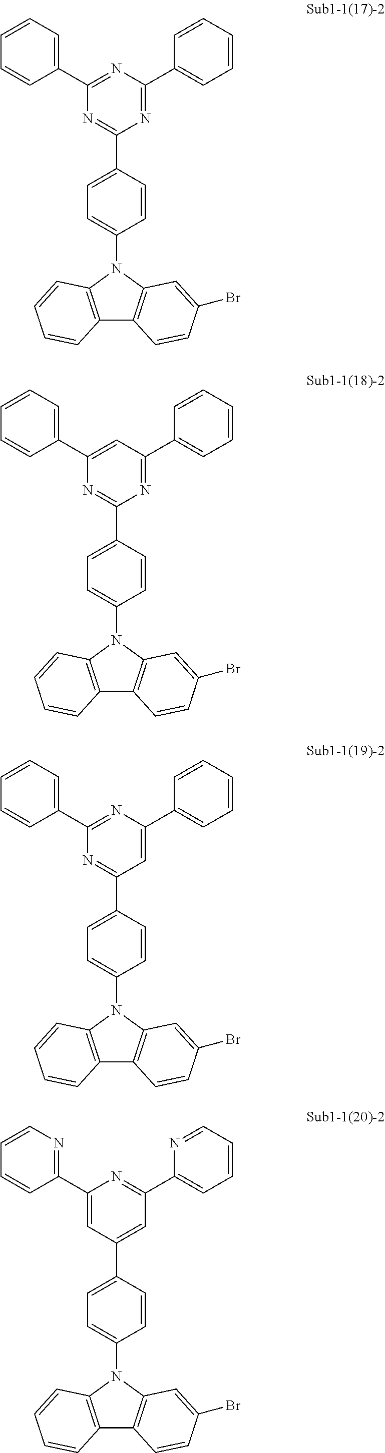 [SMILES: Brc1ccc2c3ccccc3n(-c3ccc(-c4nc(-c5ccccc5)nc(-c5ccccc5)n4)cc3)c2c1 ; Brc1ccc2c3ccccc3n(-c3ccc(-c4nc(-c5ccccc5)cc(-c5ccccc5)n4)cc3)c2c1 ; Brc1ccc2c3ccccc3n(-c3ccc(-c4cc(-c5ccccc5)nc(-c5ccccc5)n4)cc3)c2c1 ; Brc1ccc2c3ccccc3n(-c3ccc(-c4cc(-c5ccccn5)nc(-c5ccccn5)c4)cc3)c2c1]

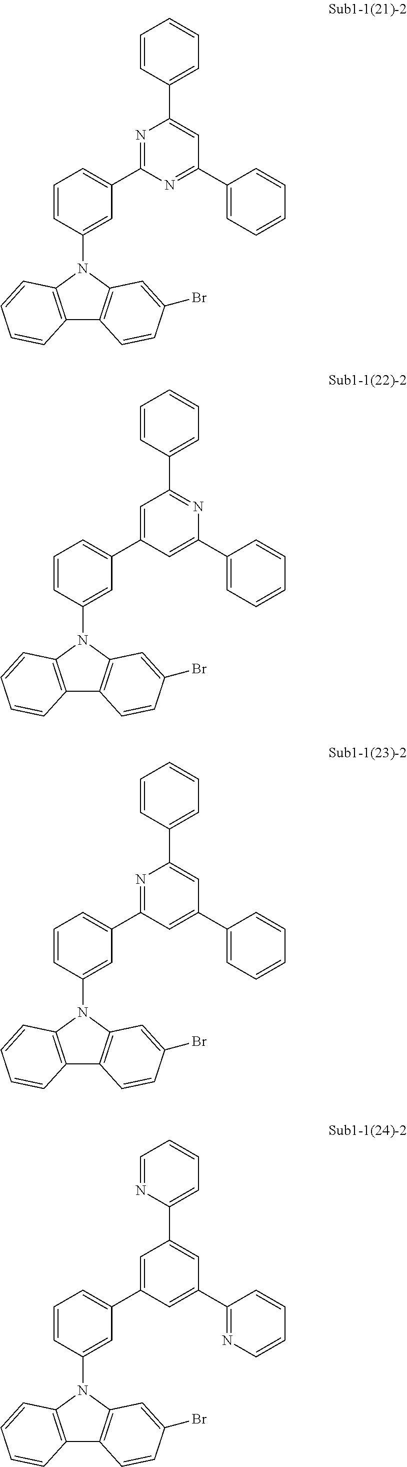 [SMILES: Brc1ccc2c3ccccc3n(-c3cccc(-c4nc(-c5ccccc5)cc(-c5ccccc5)n4)c3)c2c1 ; Brc1ccc2c3ccccc3n(-c3cccc(-c4cc(-c5ccccc5)nc(-c5ccccc5)c4)c3)c2c1 ; Brc1ccc2c3ccccc3n(-c3cccc(-c4cc(-c5ccccc5)cc(-c5ccccc5)n4)c3)c2c1 ; Brc1ccc2c3ccccc3n(-c3cccc(-c4cc(-c5ccccn5)cc(-c5ccccn5)c4)c3)c2c1]

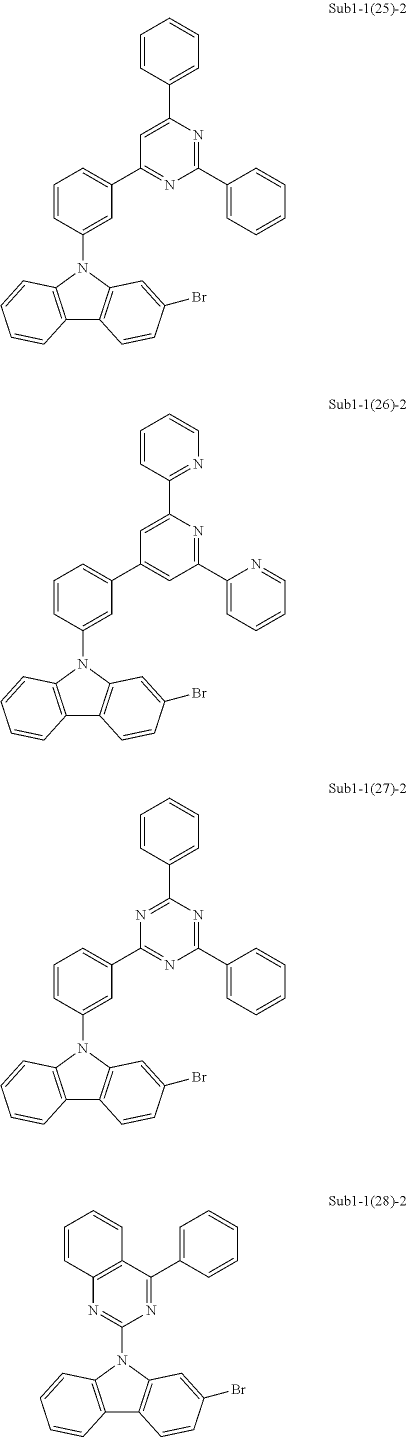 [SMILES: Brc1ccc2c3ccccc3n(-c3cccc(-c4cc(-c5ccccc5)nc(-c5ccccc5)n4)c3)c2c1 ; Brc1ccc2c3ccccc3n(-c3cccc(-c4cc(-c5ccccn5)nc(-c5ccccn5)c4)c3)c2c1 ; Brc1ccc2c3ccccc3n(-c3cccc(-c4nc(-c5ccccc5)nc(-c5ccccc5)n4)c3)c2c1 ; Brc1ccc2c3ccccc3n(-c3nc(-c4ccccc4)c4ccccc4n3)c2c1]

TABLE 2-1

| Compound | FD-MS | Compound | FD-MS |
|---|---|---|---|
| Sub1-1(1)-2 | m/z = 321.02(C₁₈H₁₂BrN = 322.20) | Sub1-1(2)-2 | m/z = 371.03(C₂₂H₁₄BrN = 372.26) |
| Sub1-1(3)-2 | m/z = 397.05(C₂₄H₁₆BrN = 398.29) | Sub1-1(4)-2 | m/z = 397.05(C₂₄H₁₆BrN = 398.29) |
| Sub1-1(5)-2 | m/z = 476.06(C₂₇H₁₇BrN₄ = 477.35) | Sub1-1(6)-2 | m/z = 475.07(C₂₈H₁₈BrN₃ = 476.37) |
| Sub1-1(7)-2 | m/z = 475.07(C₂₈H₁₈BrN₃ = 476.37) | Sub1-1(8)-2 | m/z = 475.07(C₂₈H₁₈BrN₃ = 476.37) |
| Sub1-1(9)-2 | m/z = 474.07(C₂₉H₁₉BrN₂ = 475.38) | Sub1-1(10)-2 | m/z = 474.07(C₂₉H₁₉BrN₂ = 475.38) |
| Sub1-1(11)-2 | m/z = 475.07(C₂₈H₁₈BrN₃ = 476.37) | Sub1-1(12)-2 | m/z = 476.06(C₂₇H₁₇BrN₄ = 477.35) |
| Sub1-1(13)-2 | m/z = 551.10(C₃₄H₂₂BrN₃ = 552.46) | Sub1-1(14)-2 | m/z = 550.10(C₃₅H₂₃BrN₂ = 551.47) |
| Sub1-1(15)-2 | m/z = 550.10(C₃₅H₂₃BrN₂ = 551.47) | Sub1-1(16)-2 | m/z = 551.10(C₃₄H₂₂BrN₃ = 552.46) |
| Sub1-1(17)-2 | m/z = 552.09(C₃₃H₂₁BrN₄ = 553.45) | Sub1-1(18)-2 | m/z = 551.10(C₃₄H₂₂BrN₃ = 552.46) |
| Sub1-1(19)-2 | m/z = 551.10(C₃₄H₂₂BrN₃ = 552.46) | Sub1-1(20)-2 | m/z = 552.09(C₃₃H₂₁BrN₄ = 553.45) |
| Sub1-1(21)-2 | m/z = 551.10(C₃₄H₂₂BrN₃ = 552.46) | Sub1-1(22)-2 | m/z = 550.10(C₃₅H₂₃BrN₂ = 551.47) |
| Sub1-1(23)-2 | m/z = 550.10(C₃₅H₂₃BrN₂ = 551.47) | Sub1-1(24)-2 | m/z = 551.10(C₃₄H₂₂BrN₃ = 552.46) |
| Sub1-1(25)-2 | m/z = 551.10(C₃₄H₂₂BrN₃ = 552.46) | Sub1-1(26)-2 | m/z = 552.09(C₃₃H₂₁BrN₄ = 553.45) |
| Sub1-1(27)-2 | m/z = 552.09(C₃₃H₂₁BrN₄ = 553.45) | Sub1-1(28)-2 | m/z = 449.05(C₂₆H₁₆BrN₃ = 450.33) |

### Synthesis of Sub 1-2

A two-necked RBF was equipped with a dropping-funnel, and the product was dissolved in 500 ml of THF and the temperature was maintained at −78□. After stirring for 1 h, trimethoxyborate was slowly added dropwise, followed by again stirring for 1 h. Upon the completion of the reaction, 500 ml of 5% hydrochloric acid was added, followed by stirring at room temperature for 1 h, extraction with water and ethyl acetate, concentration, and recrystallization with MC and Hexane, thereby obtaining compound Sub 2-1.

### Synthesis of Sub 1(1)-2

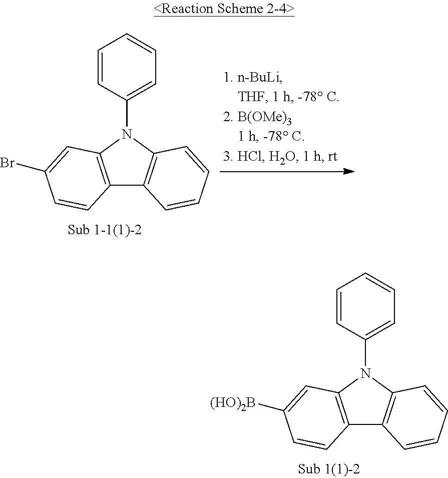 [SMILES: Brc1ccc2c3ccccc3n(-c3ccccc3)c2c1 ; OB(O)c1ccc2c3ccccc3n(-c3ccccc3)c2c1]

A two-necked RBF was equipped with a dropping-funnel, and Sub 1-1(1)-2 (38 g, 118 mmol) was dissolved in 500 ml of THF and the temperature was maintained at −78□. After stirring for 1 h, trimethoxyborate (18.4 g, 177 mmol) was slowly added dropwise, followed by again stirring for 1 h. Upon the completion of the reaction, 500 ml of 5% hydrochloric acid was added, followed by stirring at room temperature for 1 h, extraction with water and ethyl acetate, concentration, and recrystallization with MC and Hexane, thereby obtaining 21 g of compound Sub 1(1)-2 (yield: 62%).

Examples of Sub 1-2 are as follows, but are limited thereto, and FD-MS values thereof are shown in table 2-2

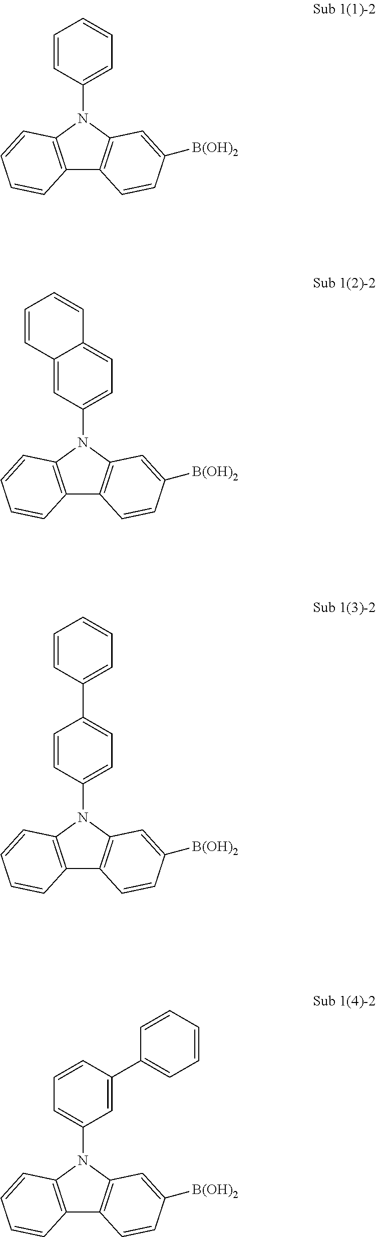 [SMILES: OB(O)c1ccc2c3ccccc3n(-c3ccccc3)c2c1 ; OB(O)c1ccc2c3ccccc3n(-c3ccc4ccccc4c3)c2c1 ; OB(O)c1ccc2c3ccccc3n(-c3ccc(-c4ccccc4)cc3)c2c1 ; OB(O)c1ccc2c3ccccc3n(-c3cccc(-c4ccccc4)c3)c2c1]

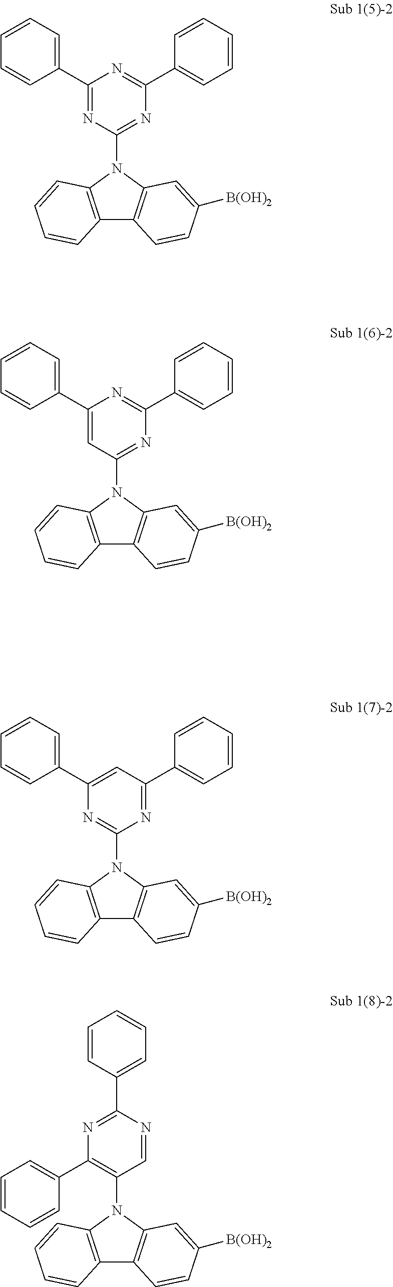 [SMILES: OB(O)c1ccc2c3ccccc3n(-c3nc(-c4ccccc4)nc(-c4ccccc4)n3)c2c1 ; OB(O)c1ccc2c3ccccc3n(-c3cc(-c4ccccc4)nc(-c4ccccc4)n3)c2c1 ; OB(O)c1ccc2c3ccccc3n(-c3nc(-c4ccccc4)cc(-c4ccccc4)n3)c2c1 ; OB(O)c1ccc2c3ccccc3n(-c3cnc(-c4ccccc4)nc3-c3ccccc3)c2c1]

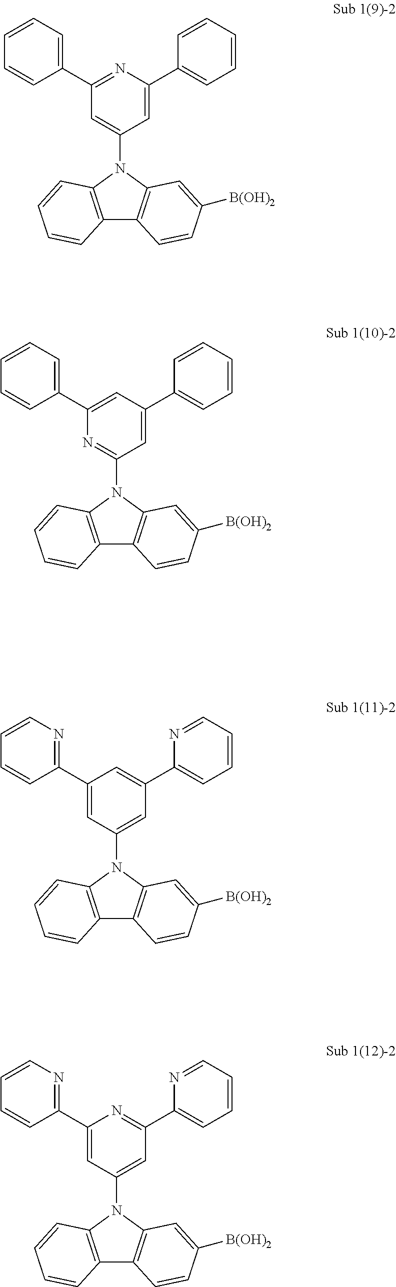 [SMILES: OB(O)c1ccc2c3ccccc3n(-c3cc(-c4ccccc4)nc(-c4ccccc4)c3)c2c1 ; OB(O)c1ccc2c3ccccc3n(-c3cc(-c4ccccc4)cc(-c4ccccc4)n3)c2c1 ; OB(O)c1ccc2c3ccccc3n(-c3cc(-c4ccccn4)cc(-c4ccccn4)c3)c2c1 ; OB(O)c1ccc2c3ccccc3n(-c3cc(-c4ccccn4)nc(-c4ccccn4)c3)c2c1]

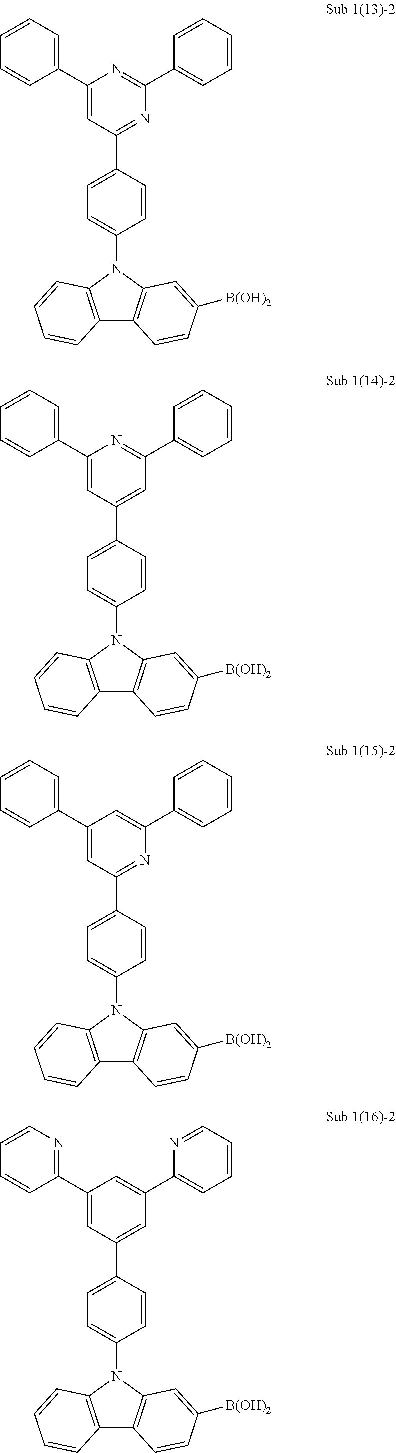 [SMILES: OB(O)c1ccc2c3ccccc3n(-c3ccc(-c4cc(-c5ccccc5)nc(-c5ccccc5)n4)cc3)c2c1 ; OB(O)c1ccc2c3ccccc3n(-c3ccc(-c4cc(-c5ccccc5)nc(-c5ccccc5)c4)cc3)c2c1 ; OB(O)c1ccc2c3ccccc3n(-c3ccc(-c4cc(-c5ccccc5)cc(-c5ccccc5)n4)cc3)c2c1 ; OB(O)c1ccc2c3ccccc3n(-c3ccc(-c4cc(-c5ccccn5)cc(-c5ccccn5)c4)cc3)c2c1]

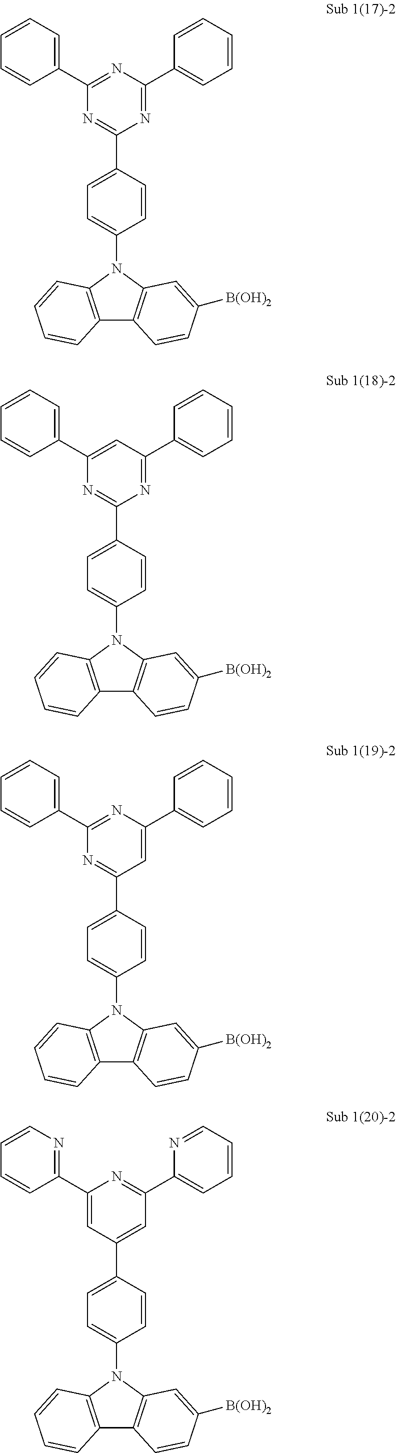 [SMILES: OB(O)c1ccc2c3ccccc3n(-c3ccc(-c4nc(-c5ccccc5)nc(-c5ccccc5)n4)cc3)c2c1 ; OB(O)c1ccc2c3ccccc3n(-c3ccc(-c4nc(-c5ccccc5)cc(-c5ccccc5)n4)cc3)c2c1 ; OB(O)c1ccc2c3ccccc3n(-c3ccc(-c4cc(-c5ccccc5)nc(-c5ccccc5)n4)cc3)c2c1 ; OB(O)c1ccc2c3ccccc3n(-c3ccc(-c4cc(-c5ccccn5)nc(-c5ccccn5)c4)cc3)c2c1]

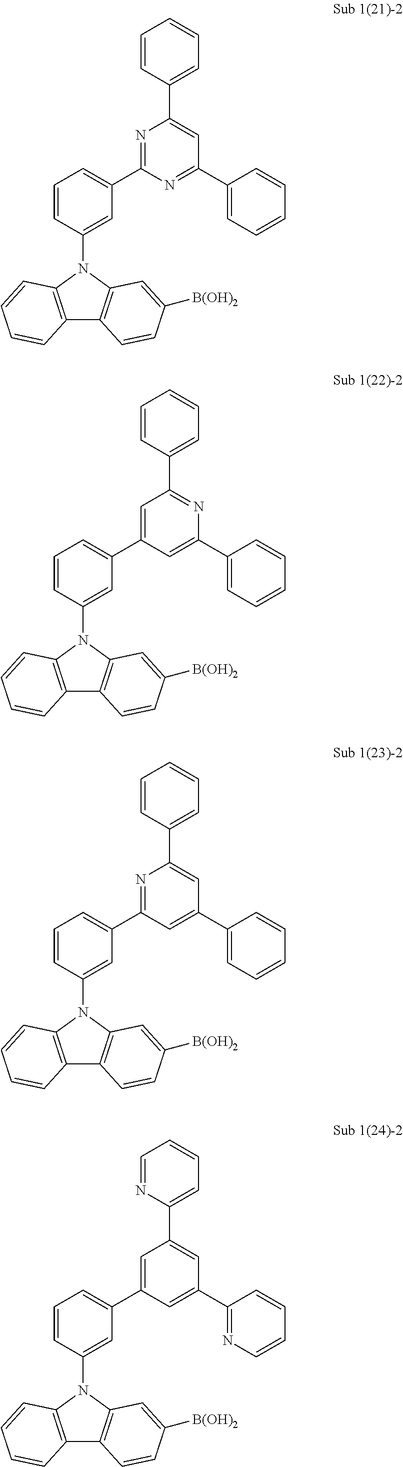 [SMILES: OB(O)c1ccc2c3ccccc3n(-c3cccc(-c4nc(-c5ccccc5)cc(-c5ccccc5)n4)c3)c2c1 ; OB(O)c1ccc2c3ccccc3n(-c3cccc(-c4cc(-c5ccccc5)nc(-c5ccccc5)c4)c3)c2c1 ; OB(O)c1ccc2c3ccccc3n(-c3cccc(-c4cc(-c5ccccc5)cc(-c5ccccc5)n4)c3)c2c1 ; OB(O)c1ccc2c3ccccc3n(-c3cccc(-c4cc(-c5ccccn5)cc(-c5ccccn5)c4)c3)c2c1]

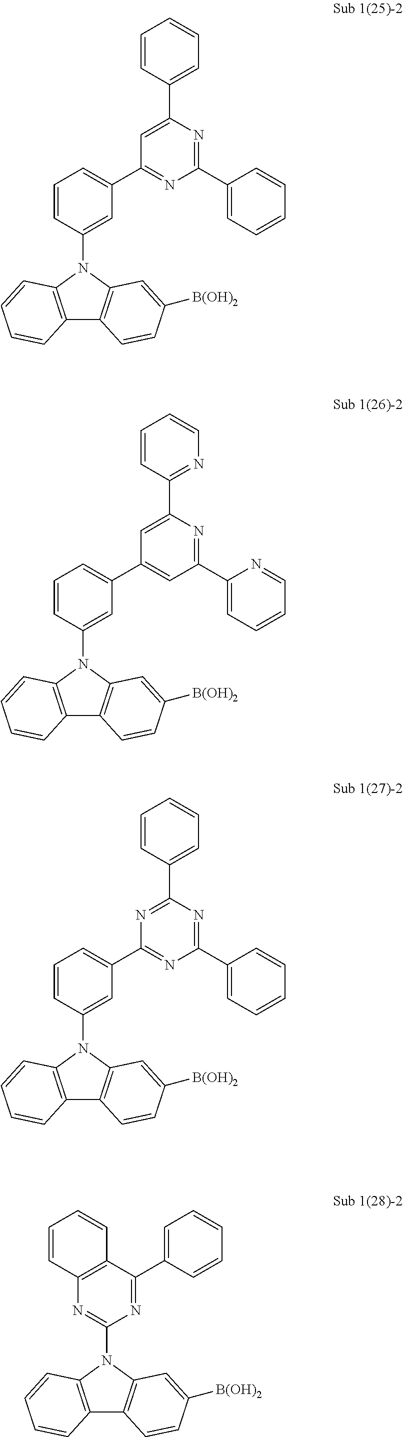 [SMILES: OB(O)c1ccc2c3ccccc3n(-c3cccc(-c4cc(-c5ccccc5)nc(-c5ccccc5)n4)c3)c2c1 ; OB(O)c1ccc2c3ccccc3n(-c3cccc(-c4cc(-c5ccccn5)nc(-c5ccccn5)c4)c3)c2c1 ; OB(O)c1ccc2c3ccccc3n(-c3cccc(-c4nc(-c5ccccc5)nc(-c5ccccc5)n4)c3)c2c1 ; OB(O)c1ccc2c3ccccc3n(-c3nc(-c4ccccc4)c4ccccc4n3)c2c1]

TABLE 2-2

| Compound | FD-MS | Compound | FD-MS |
|---|---|---|---|
| Sub 1(1)-2 | m/z = 287.11(C₁₆H₁₄BNO₃ = 287.12) | Sub 1(2)-2 | m/z = 337.13(C₂₂H₁₆BNO₂ = 337.18) |
| Sub 1(3)-2 | m/z = 363.14(C₂₄H₁₈BNO₂ = 363.22) | Sub 1(4)-2 | m/z = 363.14(C₂₄H₁₈BNO₂ = 363.22) |
| Sub 1(5)-2 | m/z = 442.16(C₂₇H₁₉BN₄O₂ = 442.28) | Sub 1(6)-2 | m/z = 441.16(C₂₈H₂₀BN₃O₂ = 441.29) |
| Sub 1(7)-2 | m/z = 441.16(C₂₈H₂₀BN₃O₂ = 441.29) | Sub 1(8)-2 | m/z = 441.16(C₂₈H₂₀BN₃O₂ = 441.29) |
| Sub 1(9)-2 | m/z = 440.17(C₂₉H₂₁BN₂O₂ = 440.30) | Sub 1(10)-2 | m/z = 440.17(C₂₉H₂₁BN₂O₂ = 440.30) |
| Sub 1(11)-2 | m/z = 441.16(C₂₈H₂₀BN₃O₂ = 441.29) | Sub 1(12)-2 | m/z = 442.16(C₂₇H₁₉BN₄O₂ = 442.28) |
| Sub 1(13)-2 | m/z = 517.20(C₃₄H₂₄BN₃O₂ = 517.38) | Sub 1(14)-2 | m/z = 516.20(C₃₅H₂₅BN₂O₂ = 516.40) |
| Sub 1(15)-2 | m/z = 516.20(C₃₅H₂₅BN₂O₂ = 516.40) | Sub 1(16)-2 | m/z = 517.20(C₃₄H₂₄BN₃O₂ = 517.38) |
| Sub 1(17)-2 | m/z = 518.19(C₃₃H₂₃BN₄O₂ = 518.37) | Sub 1(18)-2 | m/z = 517.20(C₃₄H₂₄BN₃O₂ = 517.38) |
| Sub 1(19)-2 | m/z = 517.20(C₃₄H₂₄BN₃O₂ = 517.38) | Sub 1(20)-2 | m/z = 518.19(C₃₃H₂₃BN₄O₂ = 518.37) |
| Sub 1(21)-2 | m/z = 517.20(C₃₄H₂₄BN₃O₂ = 517.38) | Sub 1(22)-2 | m/z = 516.20(C₃₅H₂₅BN₂O₂ = 516.40) |
| Sub 1(23)-2 | m/z = 516.20(C₃₅H₂₅BN₂O₂ = 516.40) | Sub 1(24)-2 | m/z = 517.20(C₃₄H₂₄BN₃O₂ = 517.38) |
| Sub 1(25)-2 | m/z = 517.20(C₃₄H₂₄BN₃O₂ = 517.38) | Sub 1(26)-2 | m/z = 518.19(C₃₃H₂₃BN₄O₂ = 518.37) |
| Sub 1(27)-2 | m/z = 518.19(C₃₃H₂₃BN₄O₂ = 518.37) | Sub 1(28)-2 | m/z = 415.15(C₂₆H₁₈BN₃O₂ = 415.25) |

### II. Synthesis Example of Sub 2-2

Sub 2-2 in Reaction Scheme 2-1 may be synthesized via the reaction pathway of Reaction Scheme 2-5 below, but is not limited thereto.

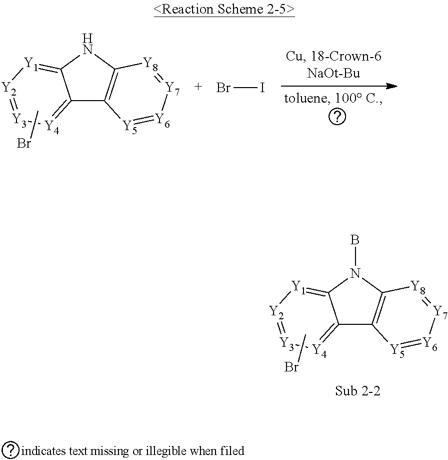

### Synthesis of Sub 1(1)-2

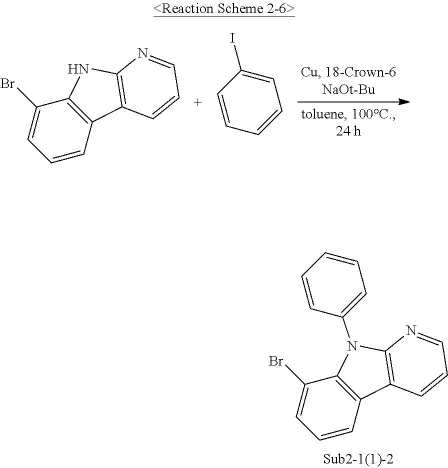

After 8-bromo-9H-pyrido[2,3-b]indole (50.2 g, 203 mmol) and iodobenzene (49.0 g, 240 mmol) were mixed with 800 mL of toluene, Cu (764 mg, 12 mmol), 18-Crown-6 (6.3 g, 24 mmol), and NaOt-Bu (57.6 g, 600 mmol) were added thereto, and the mixture was stirred under reflux at 100° C. for 24 h. After extraction with ether and water, the organic layer was dried over MgSO₄ and concentrated, and then the generated organic material was subjected to silica gel column chromatography and recrystallization to give 28.2 g of 8-bromo-9-phenyl-9H-pyrido[2,3-b]indole (yield: 43%)

Examples of Sub 2-2 are as follows, but are limited thereto, and FD-MS values thereof are shown in table 2-3

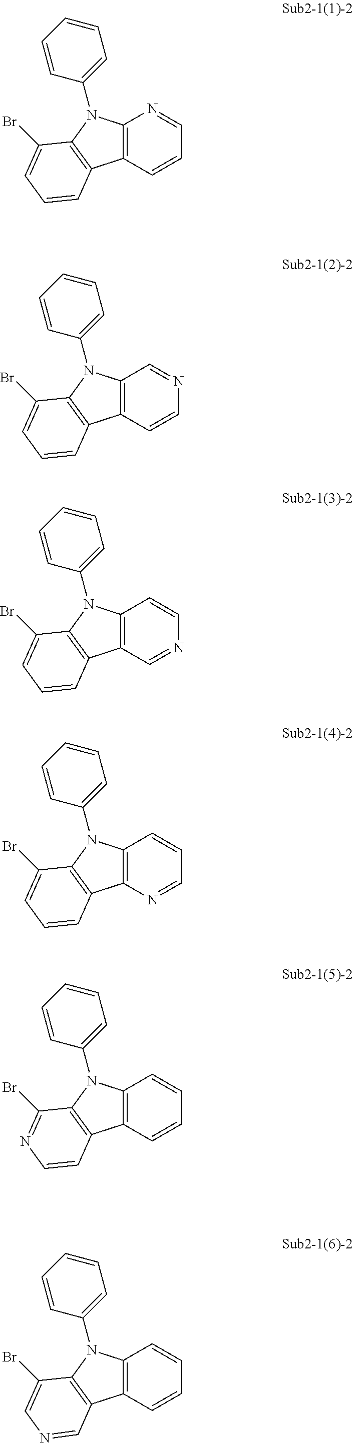 [SMILES: Brc1cccc2c3cccnc3n(-c3ccccc3)c12 ; Brc1cccc2c3ccncc3n(-c3ccccc3)c12 ; Brc1cccc2c3cnccc3n(-c3ccccc3)c12 ; Brc1cccc2c3ncccc3n(-c3ccccc3)c12 ; Brc1nccc2c3ccccc3n(-c3ccccc3)c12 ; Brc1cncc2c3ccccc3n(-c3ccccc3)c12]

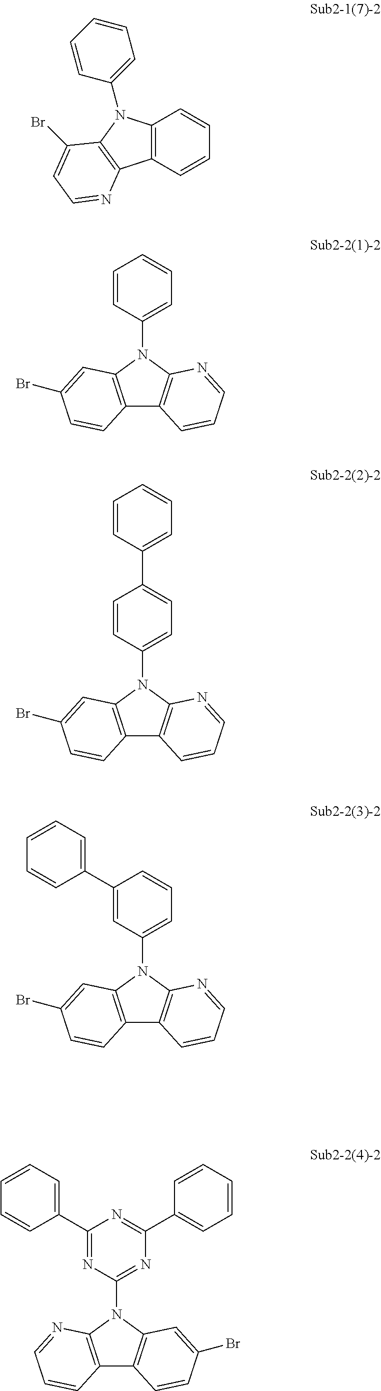 [SMILES: Brc1ccnc2c3ccccc3n(-c3ccccc3)c12 ; Brc1ccc2c3cccnc3n(-c3ccccc3)c2c1 ; Brc1ccc2c3cccnc3n(-c3ccc(-c4ccccc4)cc3)c2c1 ; Brc1ccc2c3cccnc3n(-c3cccc(-c4ccccc4)c3)c2c1 ; Brc1ccc2c3cccnc3n(-c3nc(-c4ccccc4)nc(-c4ccccc4)n3)c2c1]

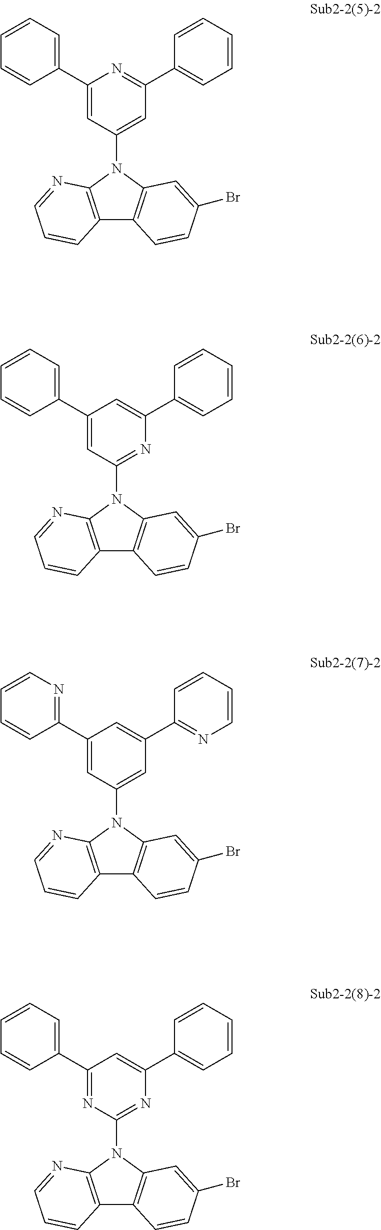 [SMILES: Brc1ccc2c3cccnc3n(-c3cc(-c4ccccc4)nc(-c4ccccc4)c3)c2c1 ; Brc1ccc2c3cccnc3n(-c3cc(-c4ccccc4)cc(-c4ccccc4)n3)c2c1 ; Brc1ccc2c3cccnc3n(-c3cc(-c4ccccn4)cc(-c4ccccn4)c3)c2c1 ; Brc1ccc2c3cccnc3n(-c3nc(-c4ccccc4)cc(-c4ccccc4)n3)c2c1]

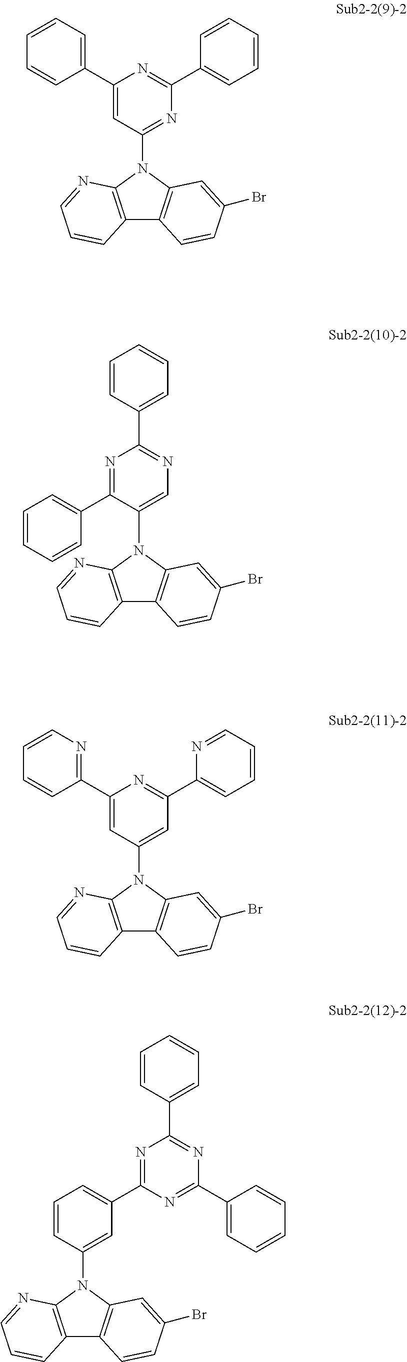 [SMILES: Brc1ccc2c3cccnc3n(-c3cc(-c4ccccc4)nc(-c4ccccc4)n3)c2c1 ; Brc1ccc2c3cccnc3n(-c3cnc(-c4ccccc4)nc3-c3ccccc3)c2c1 ; Brc1ccc2c3cccnc3n(-c3cc(-c4ccccn4)nc(-c4ccccn4)c3)c2c1 ; Brc1ccc2c3cccnc3n(-c3cccc(-c4nc(-c5ccccc5)nc(-c5ccccc5)n4)c3)c2c1]

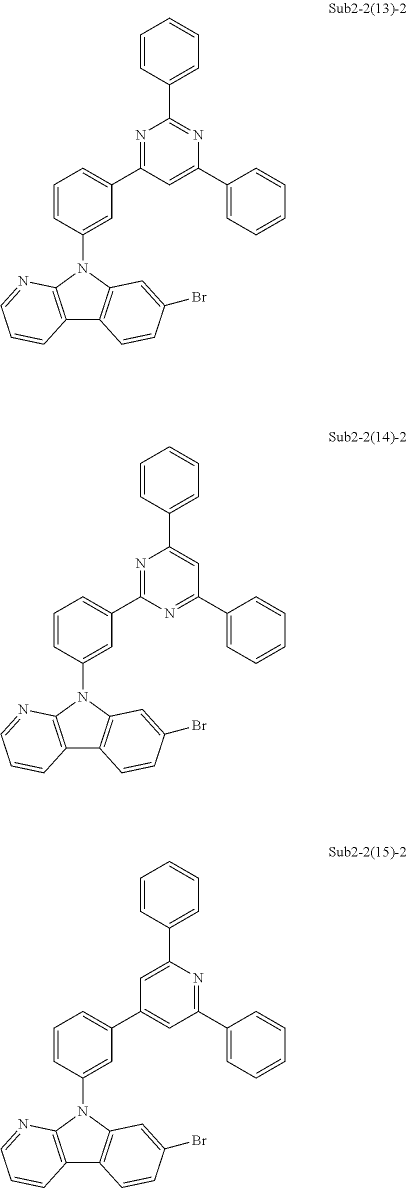 [SMILES: Brc1ccc2c3cccnc3n(-c3cccc(-c4cc(-c5ccccc5)nc(-c5ccccc5)n4)c3)c2c1 ; Brc1ccc2c3cccnc3n(-c3cccc(-c4nc(-c5ccccc5)cc(-c5ccccc5)n4)c3)c2c1 ; Brc1ccc2c3cccnc3n(-c3cccc(-c4cc(-c5ccccc5)nc(-c5ccccc5)c4)c3)c2c1]

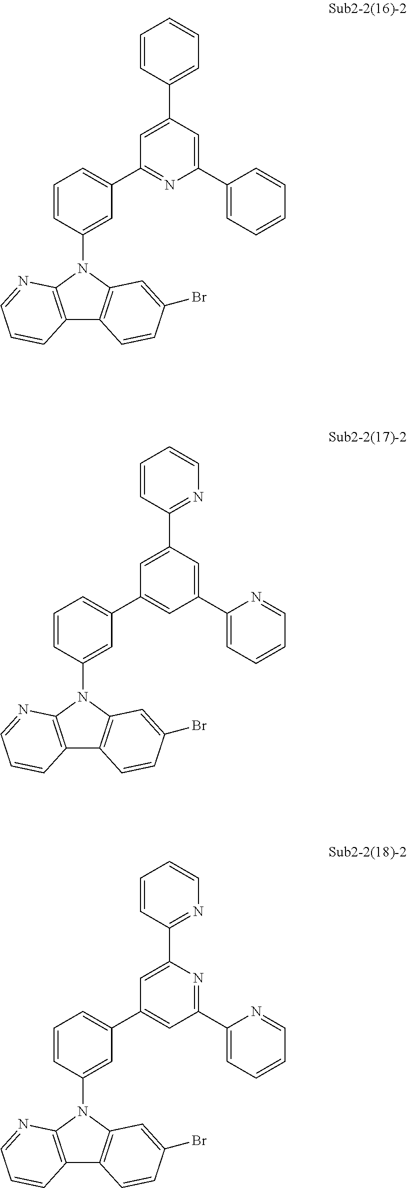 [SMILES: Brc1ccc2c3cccnc3n(-c3cccc(-c4cc(-c5ccccc5)cc(-c5ccccc5)n4)c3)c2c1 ; Brc1ccc2c3cccnc3n(-c3cccc(-c4cc(-c5ccccn5)cc(-c5ccccn5)c4)c3)c2c1 ; Brc1ccc2c3cccnc3n(-c3cccc(-c4cc(-c5ccccn5)nc(-c5ccccn5)c4)c3)c2c1]

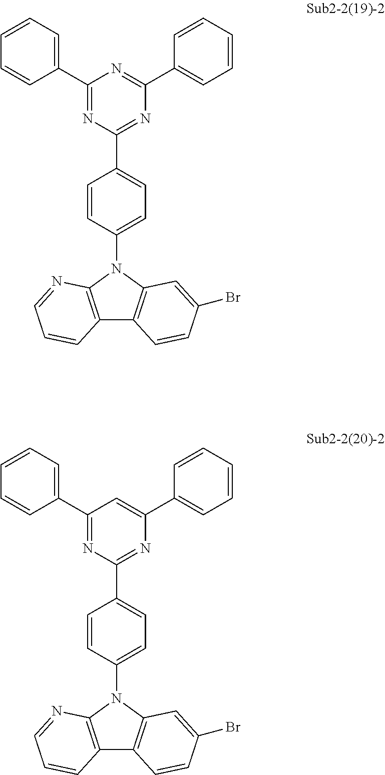 [SMILES: Brc1ccc2c3cccnc3n(-c3ccc(-c4nc(-c5ccccc5)nc(-c5ccccc5)n4)cc3)c2c1 ; Brc1ccc2c3cccnc3n(-c3ccc(-c4nc(-c5ccccc5)cc(-c5ccccc5)n4)cc3)c2c1]

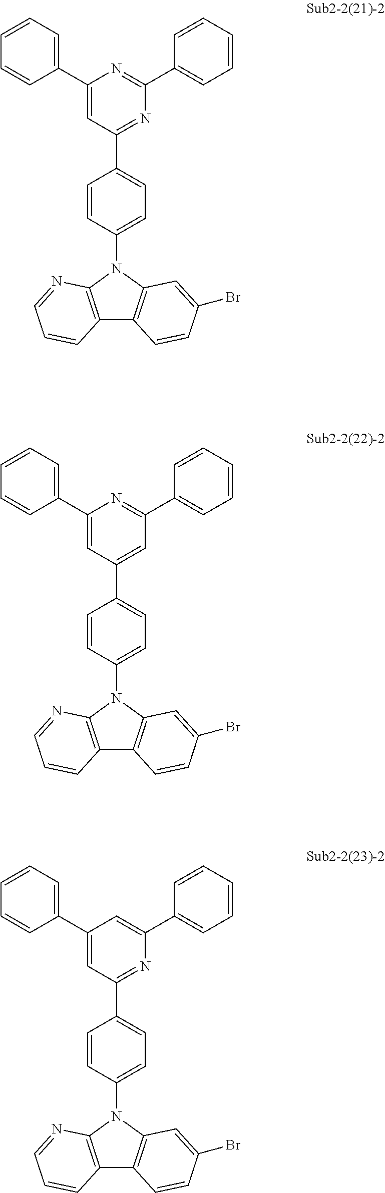 [SMILES: Brc1ccc2c3cccnc3n(-c3ccc(-c4cc(-c5ccccc5)nc(-c5ccccc5)n4)cc3)c2c1 ; Brc1ccc2c3cccnc3n(-c3ccc(-c4cc(-c5ccccc5)nc(-c5ccccc5)c4)cc3)c2c1 ; Brc1ccc2c3cccnc3n(-c3ccc(-c4cc(-c5ccccc5)cc(-c5ccccc5)n4)cc3)c2c1]

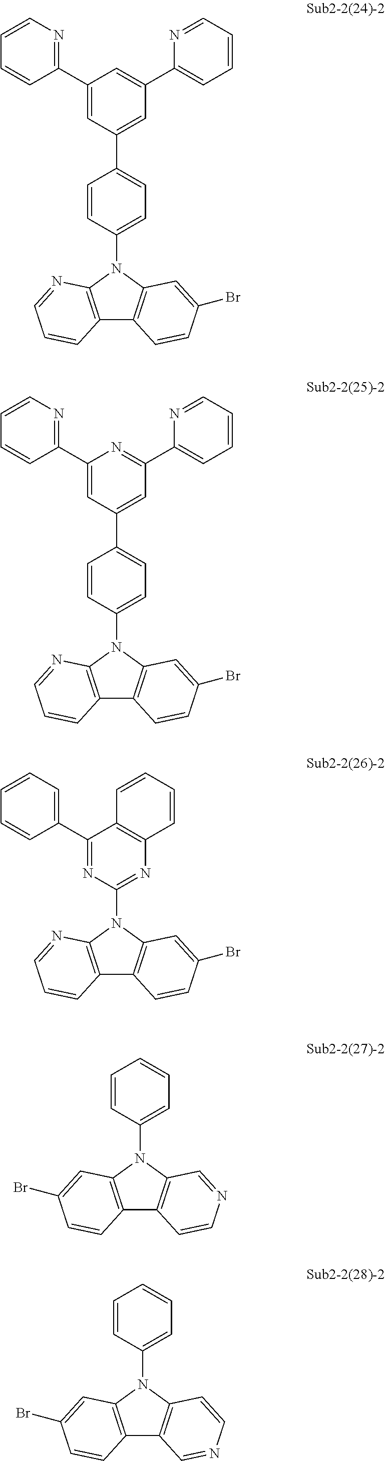 [SMILES: Brc1ccc2c3cccnc3n(-c3ccc(-c4cc(-c5ccccn5)cc(-c5ccccn5)c4)cc3)c2c1 ; Brc1ccc2c3cccnc3n(-c3ccc(-c4cc(-c5ccccn5)nc(-c5ccccn5)c4)cc3)c2c1 ; Brc1ccc2c3cccnc3n(-c3nc(-c4ccccc4)c4ccccc4n3)c2c1 ; Brc1ccc2c3ccncc3n(-c3ccccc3)c2c1 ; Brc1ccc2c3cnccc3n(-c3ccccc3)c2c1]

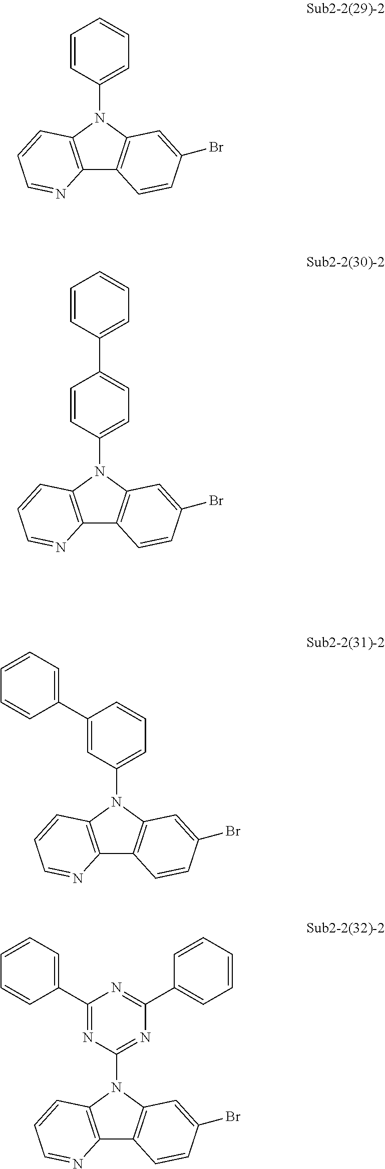 [SMILES: Brc1ccc2c3ncccc3n(-c3ccccc3)c2c1 ; Brc1ccc2c3ncccc3n(-c3ccc(-c4ccccc4)cc3)c2c1 ; Brc1ccc2c3ncccc3n(-c3cccc(-c4ccccc4)c3)c2c1 ; Brc1ccc2c3ncccc3n(-c3nc(-c4ccccc4)nc(-c4ccccc4)n3)c2c1]

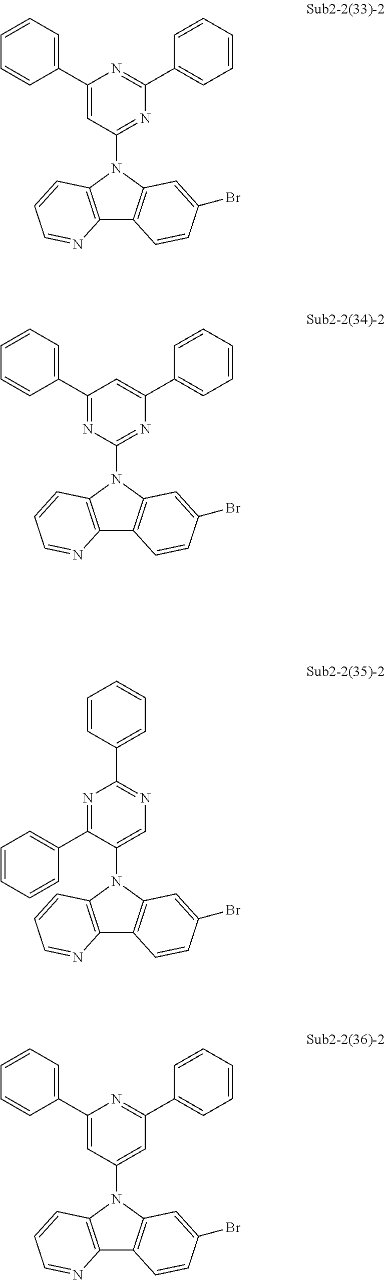 [SMILES: Brc1ccc2c3ncccc3n(-c3cc(-c4ccccc4)nc(-c4ccccc4)n3)c2c1 ; Brc1ccc2c3ncccc3n(-c3nc(-c4ccccc4)cc(-c4ccccc4)n3)c2c1 ; Brc1ccc2c3ncccc3n(-c3cnc(-c4ccccc4)nc3-c3ccccc3)c2c1 ; Brc1ccc2c3ncccc3n(-c3cc(-c4ccccc4)nc(-c4ccccc4)c3)c2c1]

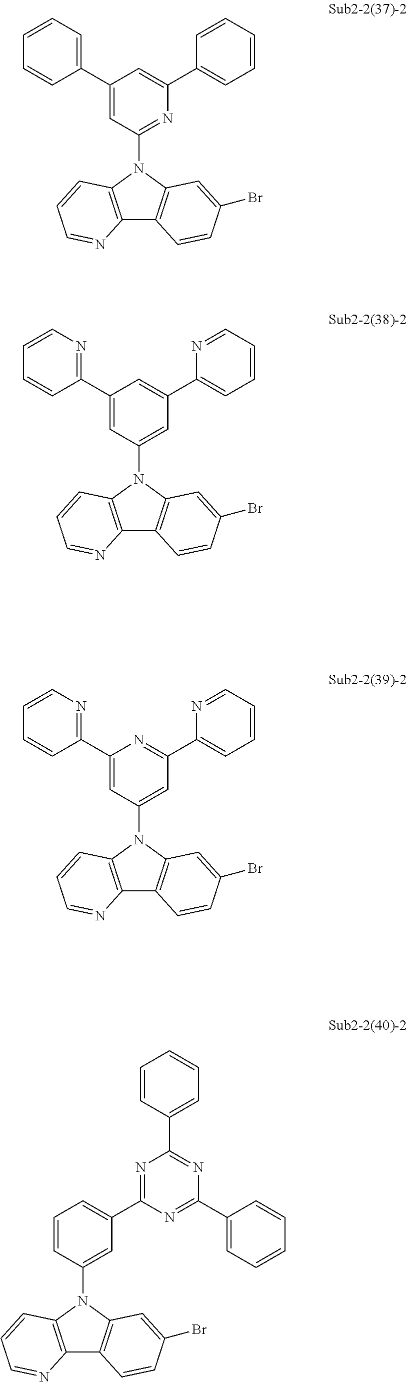 [SMILES: Brc1ccc2c3ncccc3n(-c3cc(-c4ccccc4)cc(-c4ccccc4)n3)c2c1 ; Brc1ccc2c3ncccc3n(-c3cc(-c4ccccn4)cc(-c4ccccn4)c3)c2c1 ; Brc1ccc2c3ncccc3n(-c3cc(-c4ccccn4)nc(-c4ccccn4)c3)c2c1 ; Brc1ccc2c3ncccc3n(-c3cccc(-c4nc(-c5ccccc5)nc(-c5ccccc5)n4)c3)c2c1]

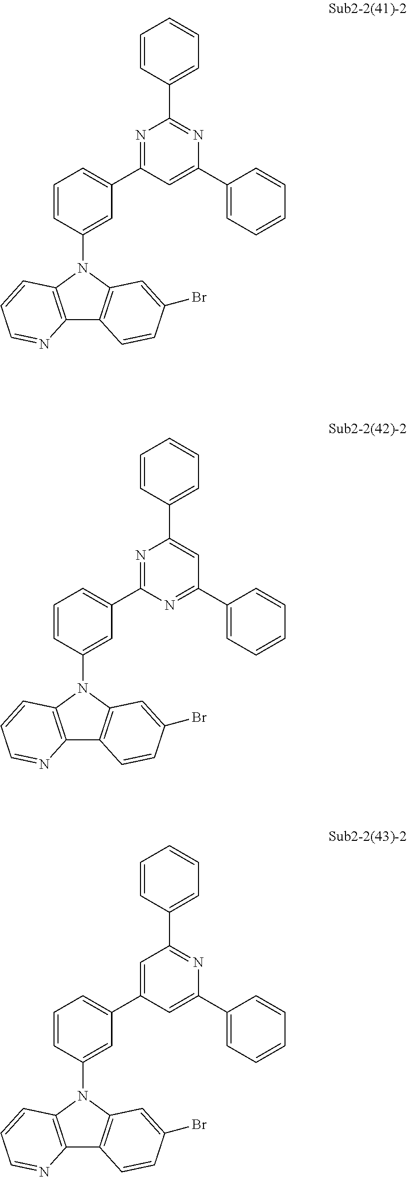 [SMILES: Brc1ccc2c3ncccc3n(-c3cccc(-c4cc(-c5ccccc5)nc(-c5ccccc5)n4)c3)c2c1 ; Brc1ccc2c3ncccc3n(-c3cccc(-c4nc(-c5ccccc5)cc(-c5ccccc5)n4)c3)c2c1 ; Brc1ccc2c3ncccc3n(-c3cccc(-c4cc(-c5ccccc5)nc(-c5ccccc5)c4)c3)c2c1]

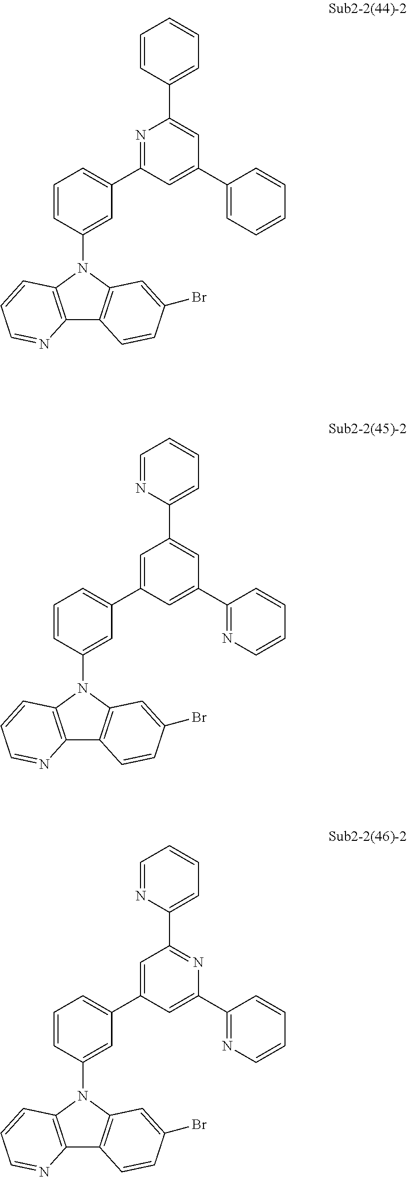 [SMILES: Brc1ccc2c3ncccc3n(-c3cccc(-c4cc(-c5ccccc5)cc(-c5ccccc5)n4)c3)c2c1 ; Brc1ccc2c3ncccc3n(-c3cccc(-c4cc(-c5ccccn5)cc(-c5ccccn5)c4)c3)c2c1 ; Brc1ccc2c3ncccc3n(-c3cccc(-c4cc(-c5ccccn5)nc(-c5ccccn5)c4)c3)c2c1]

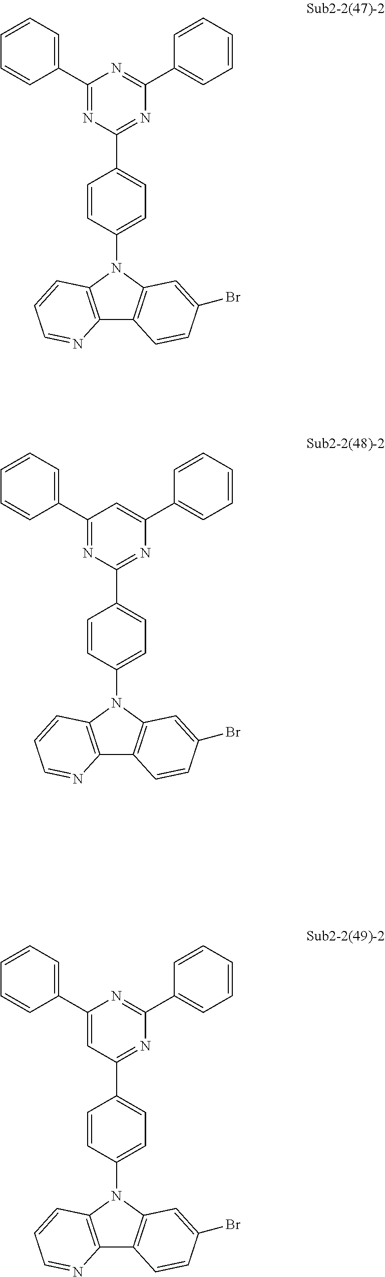 [SMILES: Brc1ccc2c3ncccc3n(-c3ccc(-c4nc(-c5ccccc5)nc(-c5ccccc5)n4)cc3)c2c1 ; Brc1ccc2c3ncccc3n(-c3ccc(-c4nc(-c5ccccc5)cc(-c5ccccc5)n4)cc3)c2c1 ; Brc1ccc2c3ncccc3n(-c3ccc(-c4cc(-c5ccccc5)nc(-c5ccccc5)n4)cc3)c2c1]

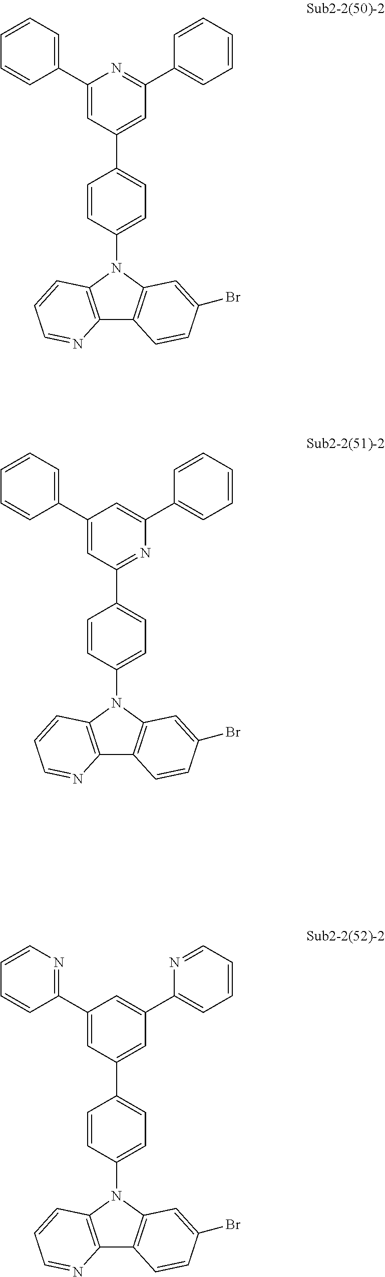 [SMILES: Brc1ccc2c3ncccc3n(-c3ccc(-c4cc(-c5ccccc5)nc(-c5ccccc5)c4)cc3)c2c1 ; Brc1ccc2c3ncccc3n(-c3ccc(-c4cc(-c5ccccc5)cc(-c5ccccc5)n4)cc3)c2c1 ; Brc1ccc2c3ncccc3n(-c3ccc(-c4cc(-c5ccccn5)cc(-c5ccccn5)c4)cc3)c2c1]

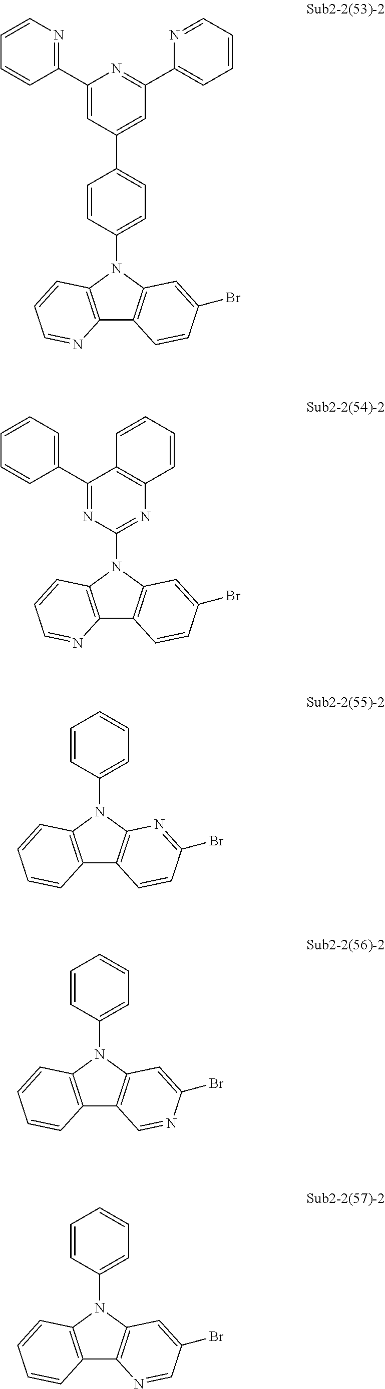 [SMILES: Brc1ccc2c3ncccc3n(-c3ccc(-c4cc(-c5ccccn5)nc(-c5ccccn5)c4)cc3)c2c1 ; Brc1ccc2c3ncccc3n(-c3nc(-c4ccccc4)c4ccccc4n3)c2c1 ; Brc1ccc2c3ccccc3n(-c3ccccc3)c2n1 ; Brc1cc2c(cn1)c1ccccc1n2-c1ccccc1 ; Brc1cnc2c3ccccc3n(-c3ccccc3)c2c1]

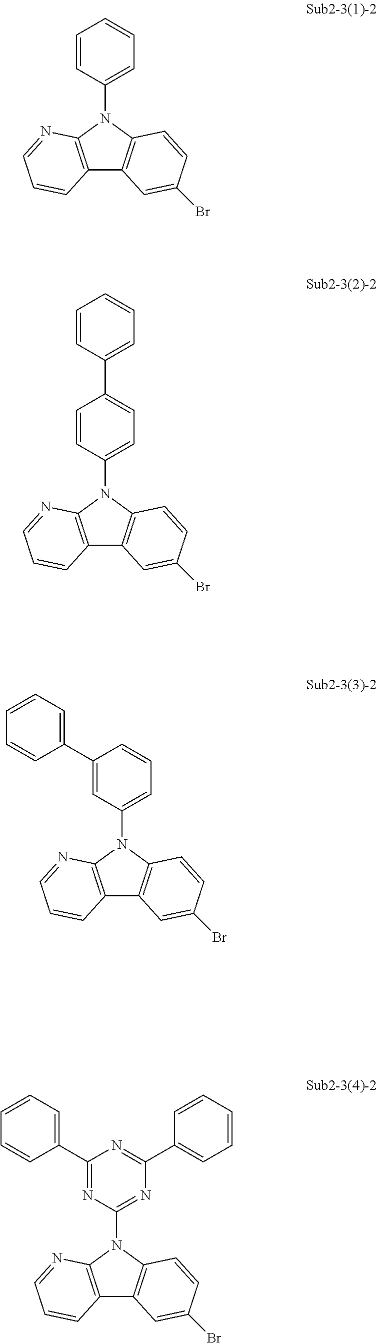 [SMILES: Brc1ccc2c(c1)c1cccnc1n2-c1ccccc1 ; Brc1ccc2c(c1)c1cccnc1n2-c1ccc(-c2ccccc2)cc1 ; Brc1ccc2c(c1)c1cccnc1n2-c1cccc(-c2ccccc2)c1 ; Brc1ccc2c(c1)c1cccnc1n2-c1nc(-c2ccccc2)nc(-c2ccccc2)n1]

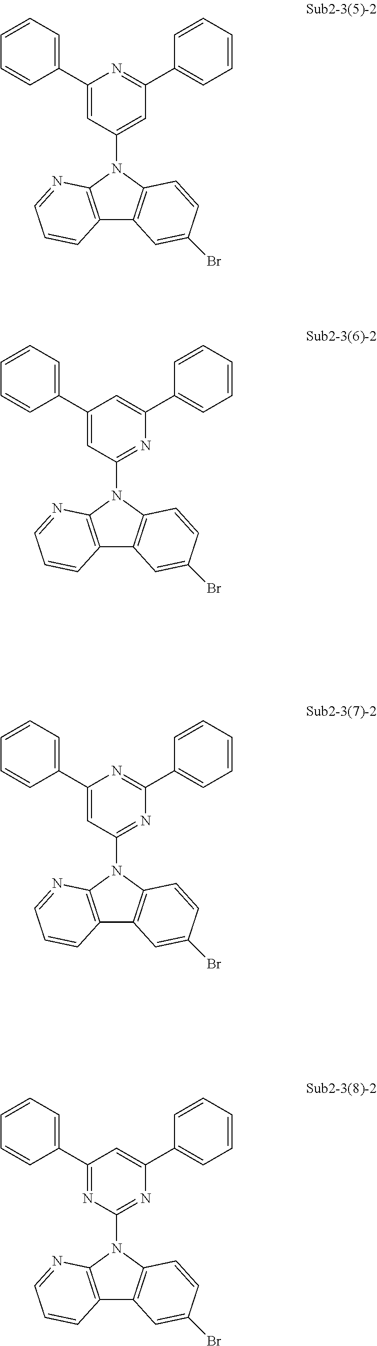 [SMILES: Brc1ccc2c(c1)c1cccnc1n2-c1cc(-c2ccccc2)nc(-c2ccccc2)c1 ; Brc1ccc2c(c1)c1cccnc1n2-c1cc(-c2ccccc2)cc(-c2ccccc2)n1 ; Brc1ccc2c(c1)c1cccnc1n2-c1cc(-c2ccccc2)nc(-c2ccccc2)n1 ; Brc1ccc2c(c1)c1cccnc1n2-c1nc(-c2ccccc2)cc(-c2ccccc2)n1]

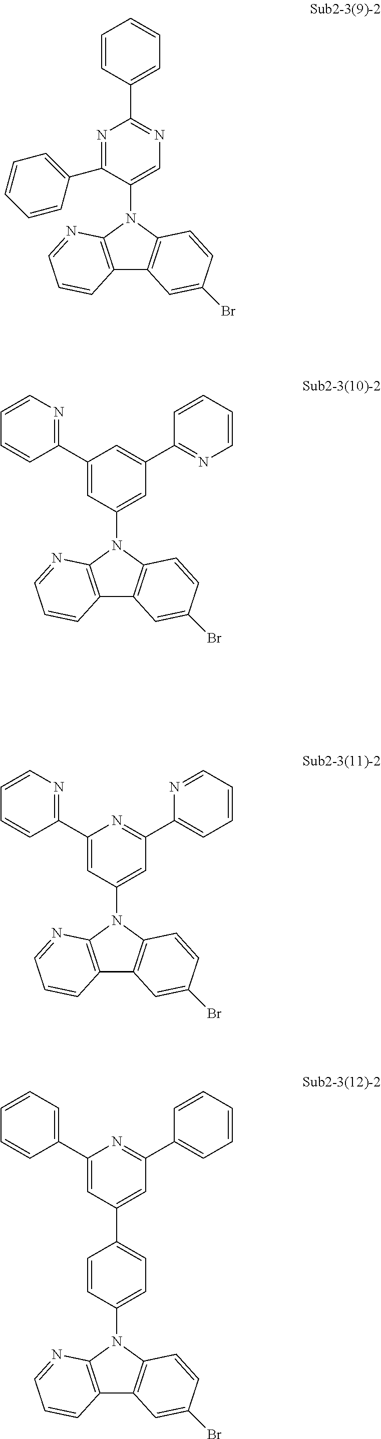 [SMILES: Brc1ccc2c(c1)c1cccnc1n2-c1cnc(-c2ccccc2)nc1-c1ccccc1 ; Brc1ccc2c(c1)c1cccnc1n2-c1cc(-c2ccccn2)cc(-c2ccccn2)c1 ; Brc1ccc2c(c1)c1cccnc1n2-c1cc(-c2ccccn2)nc(-c2ccccn2)c1 ; Brc1ccc2c(c1)c1cccnc1n2-c1ccc(-c2cc(-c3ccccc3)nc(-c3ccccc3)c2)cc1]

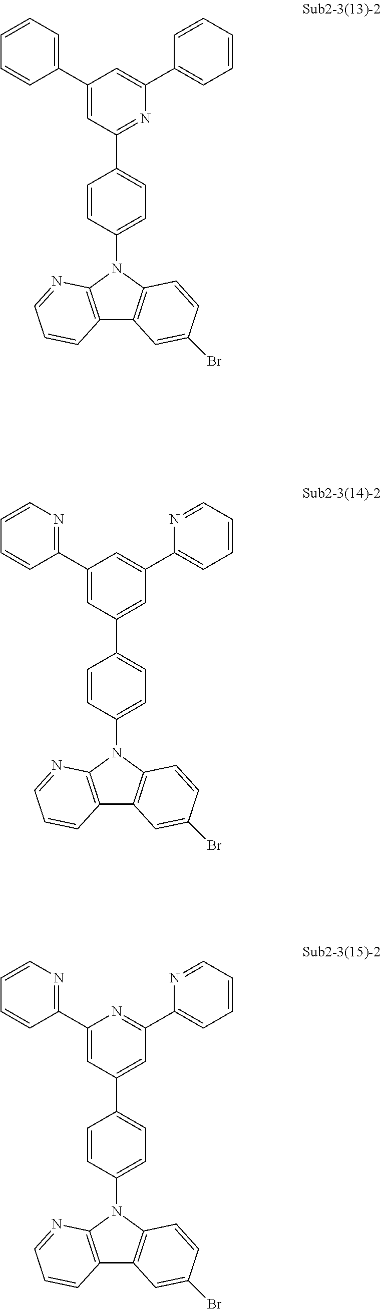 [SMILES: Brc1ccc2c(c1)c1cccnc1n2-c1ccc(-c2cc(-c3ccccc3)cc(-c3ccccc3)n2)cc1 ; Brc1ccc2c(c1)c1cccnc1n2-c1ccc(-c2cc(-c3ccccn3)cc(-c3ccccn3)c2)cc1 ; Brc1ccc2c(c1)c1cccnc1n2-c1ccc(-c2cc(-c3ccccn3)nc(-c3ccccn3)c2)cc1]

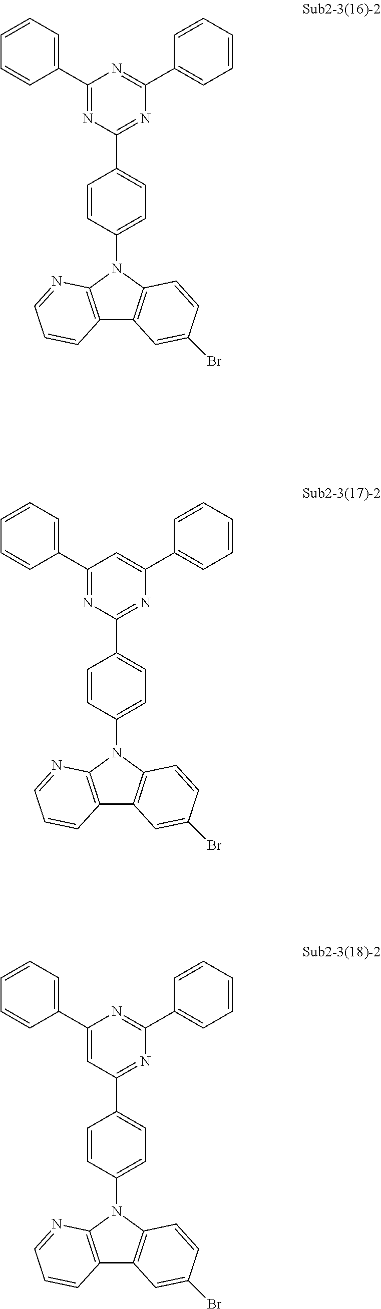 [SMILES: Brc1ccc2c(c1)c1cccnc1n2-c1ccc(-c2nc(-c3ccccc3)nc(-c3ccccc3)n2)cc1 ; Brc1ccc2c(c1)c1cccnc1n2-c1ccc(-c2nc(-c3ccccc3)cc(-c3ccccc3)n2)cc1 ; Brc1ccc2c(c1)c1cccnc1n2-c1ccc(-c2cc(-c3ccccc3)nc(-c3ccccc3)n2)cc1]

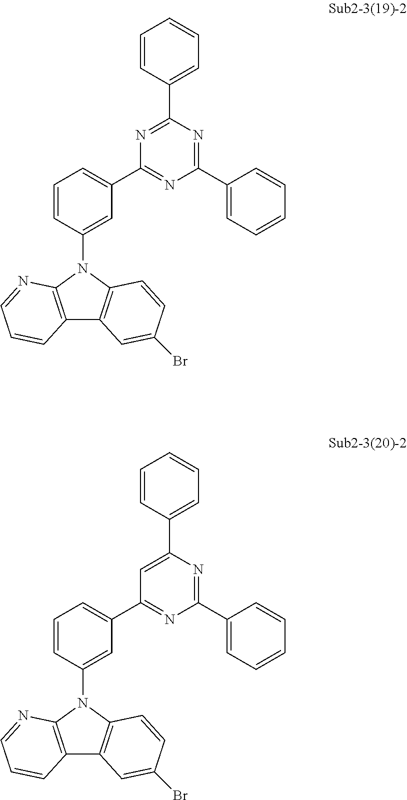 [SMILES: Brc1ccc2c(c1)c1cccnc1n2-c1cccc(-c2nc(-c3ccccc3)nc(-c3ccccc3)n2)c1 ; Brc1ccc2c(c1)c1cccnc1n2-c1cccc(-c2cc(-c3ccccc3)nc(-c3ccccc3)n2)c1]

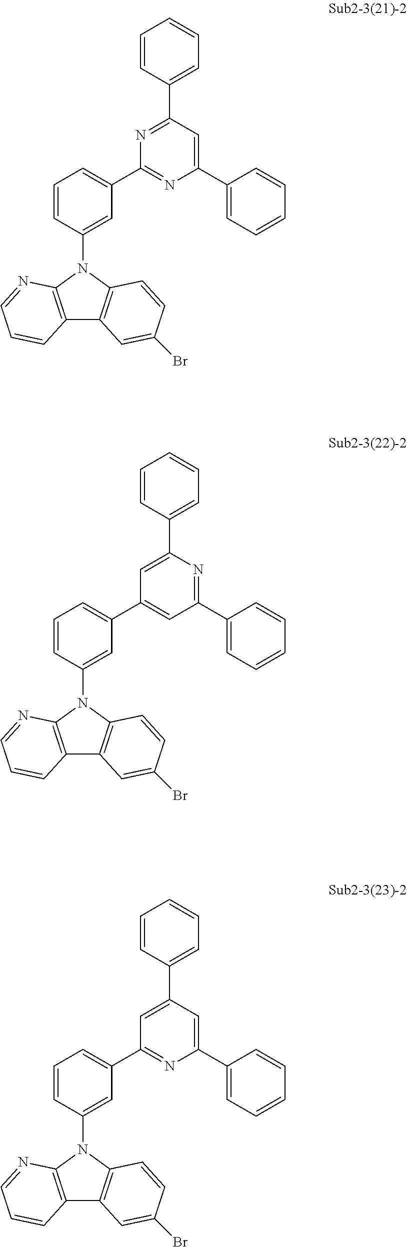 [SMILES: Brc1ccc2c(c1)c1cccnc1n2-c1cccc(-c2nc(-c3ccccc3)cc(-c3ccccc3)n2)c1 ; Brc1ccc2c(c1)c1cccnc1n2-c1cccc(-c2cc(-c3ccccc3)nc(-c3ccccc3)c2)c1 ; Brc1ccc2c(c1)c1cccnc1n2-c1cccc(-c2cc(-c3ccccc3)cc(-c3ccccc3)n2)c1]

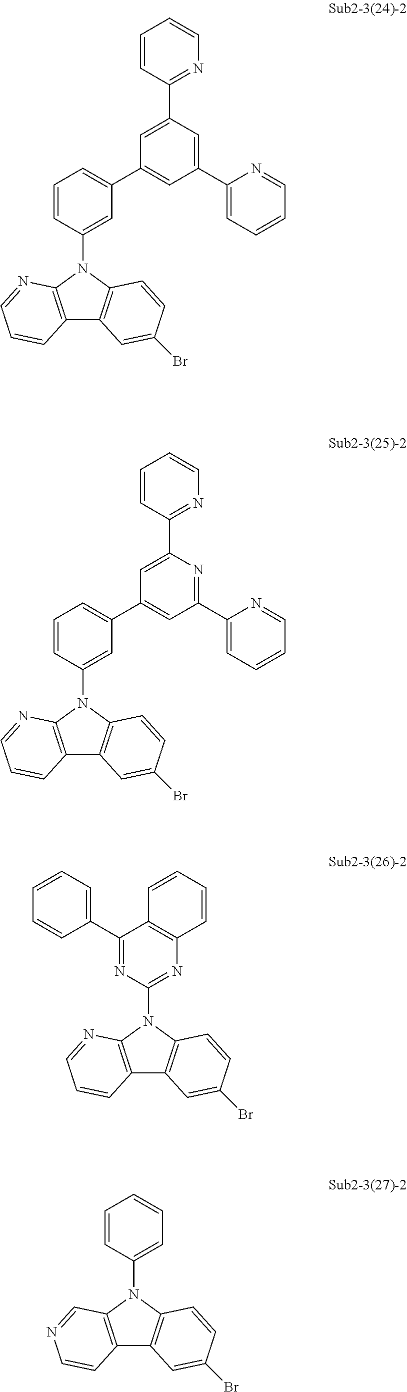 [SMILES: Brc1ccc2c(c1)c1cccnc1n2-c1cccc(-c2cc(-c3ccccn3)cc(-c3ccccn3)c2)c1 ; Brc1ccc2c(c1)c1cccnc1n2-c1cccc(-c2cc(-c3ccccn3)nc(-c3ccccn3)c2)c1 ; Brc1ccc2c(c1)c1cccnc1n2-c1nc(-c2ccccc2)c2ccccc2n1 ; Brc1ccc2c(c1)c1ccncc1n2-c1ccccc1]

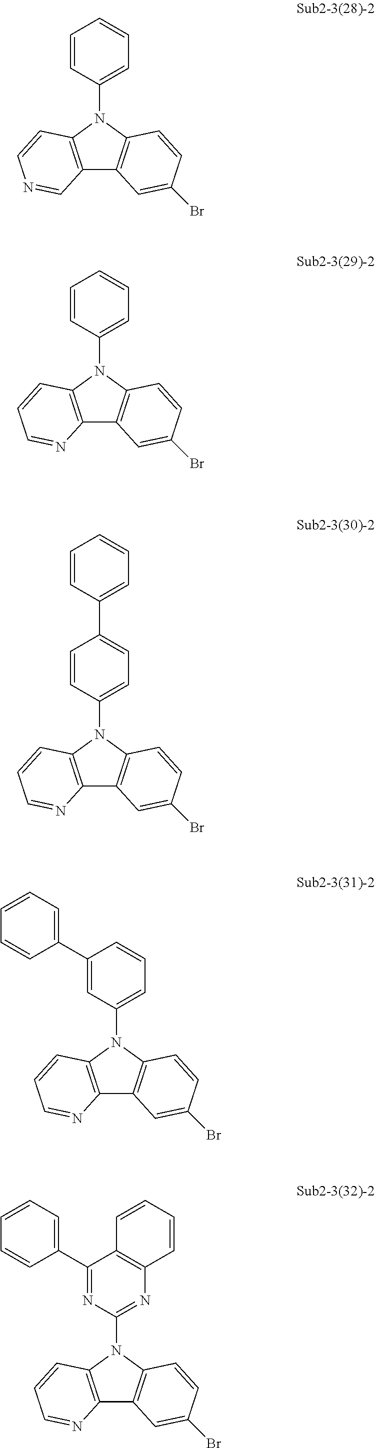 [SMILES: Brc1ccc2c(c1)c1cnccc1n2-c1ccccc1 ; Brc1ccc2c(c1)c1ncccc1n2-c1ccccc1 ; Brc1ccc2c(c1)c1ncccc1n2-c1ccc(-c2ccccc2)cc1 ; Brc1ccc2c(c1)c1ncccc1n2-c1cccc(-c2ccccc2)c1 ; Brc1ccc2c(c1)c1ncccc1n2-c1nc(-c2ccccc2)c2ccccc2n1]

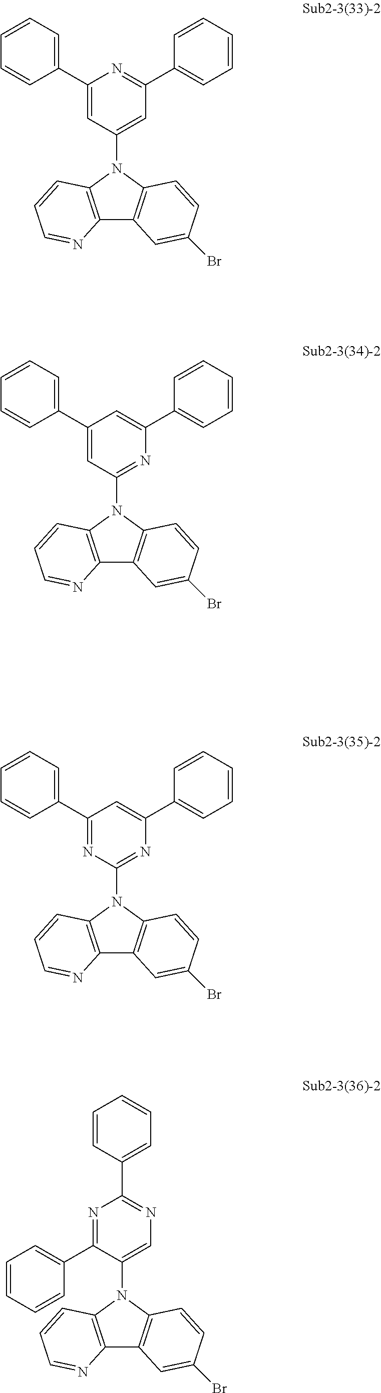 [SMILES: Brc1ccc2c(c1)c1ncccc1n2-c1cc(-c2ccccc2)nc(-c2ccccc2)c1 ; Brc1ccc2c(c1)c1ncccc1n2-c1cc(-c2ccccc2)cc(-c2ccccc2)n1 ; Brc1ccc2c(c1)c1ncccc1n2-c1nc(-c2ccccc2)cc(-c2ccccc2)n1 ; Brc1ccc2c(c1)c1ncccc1n2-c1cnc(-c2ccccc2)nc1-c1ccccc1]

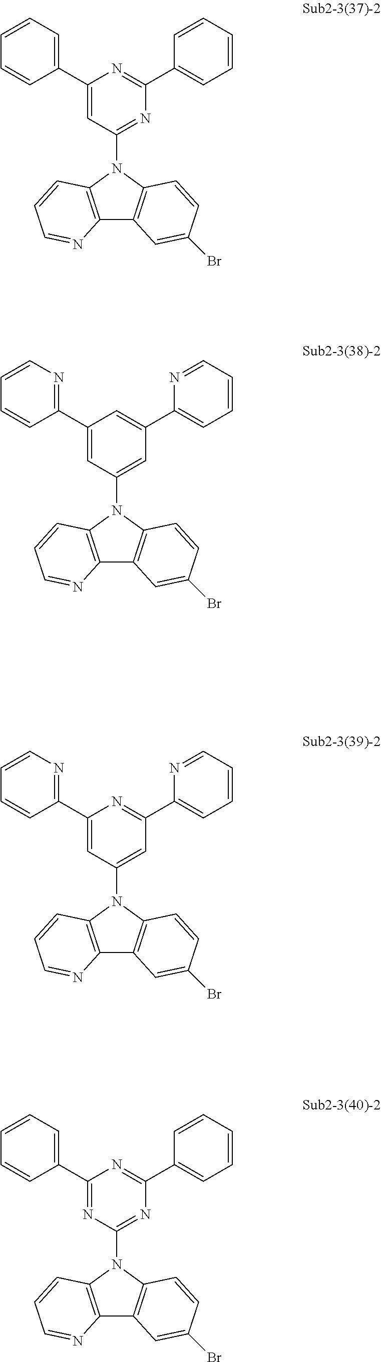 [SMILES: Brc1ccc2c(c1)c1ncccc1n2-c1cc(-c2ccccc2)nc(-c2ccccc2)n1 ; Brc1ccc2c(c1)c1ncccc1n2-c1cc(-c2ccccn2)cc(-c2ccccn2)c1 ; Brc1ccc2c(c1)c1ncccc1n2-c1cc(-c2ccccn2)nc(-c2ccccn2)c1 ; Brc1ccc2c(c1)c1ncccc1n2-c1nc(-c2ccccc2)nc(-c2ccccc2)n1]

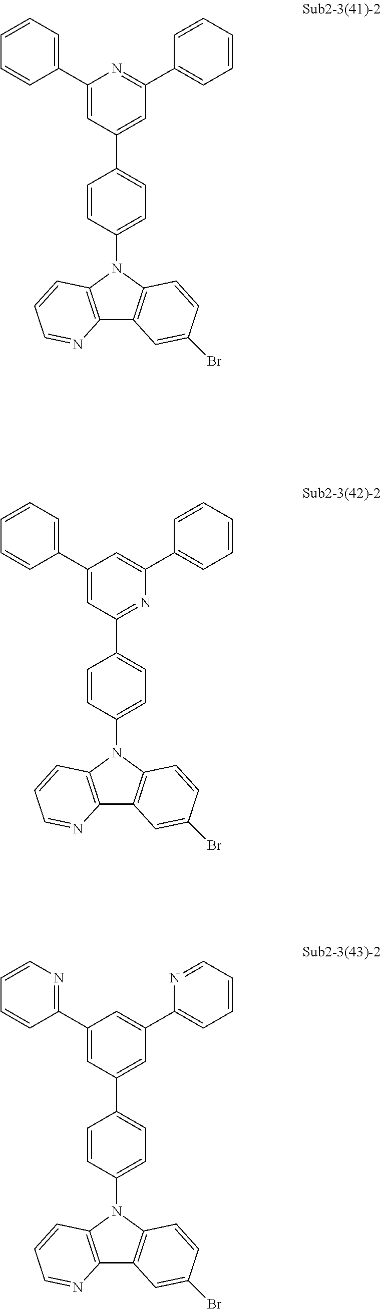 [SMILES: Brc1ccc2c(c1)c1ncccc1n2-c1ccc(-c2cc(-c3ccccc3)nc(-c3ccccc3)c2)cc1 ; Brc1ccc2c(c1)c1ncccc1n2-c1ccc(-c2cc(-c3ccccc3)cc(-c3ccccc3)n2)cc1 ; Brc1ccc2c(c1)c1ncccc1n2-c1ccc(-c2cc(-c3ccccn3)cc(-c3ccccn3)c2)cc1]

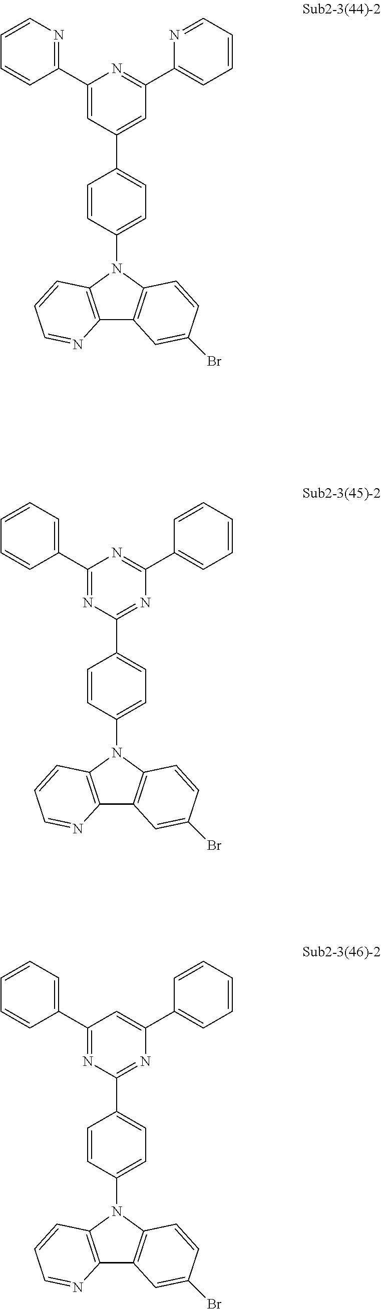 [SMILES: Brc1ccc2c(c1)c1ncccc1n2-c1ccc(-c2cc(-c3ccccn3)nc(-c3ccccn3)c2)cc1 ; Brc1ccc2c(c1)c1ncccc1n2-c1ccc(-c2nc(-c3ccccc3)nc(-c3ccccc3)n2)cc1 ; Brc1ccc2c(c1)c1ncccc1n2-c1ccc(-c2nc(-c3ccccc3)cc(-c3ccccc3)n2)cc1]

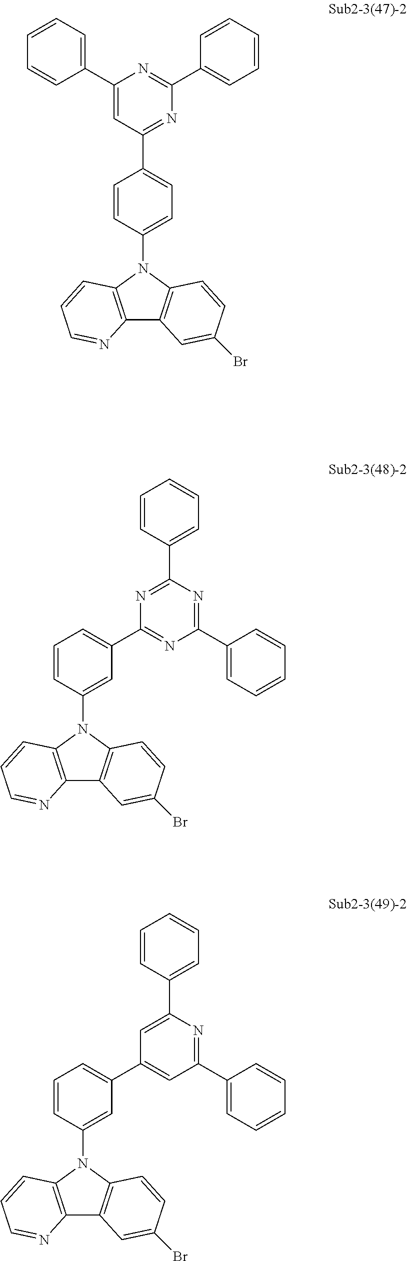 [SMILES: Brc1ccc2c(c1)c1ncccc1n2-c1ccc(-c2cc(-c3ccccc3)nc(-c3ccccc3)n2)cc1 ; Brc1ccc2c(c1)c1ncccc1n2-c1cccc(-c2nc(-c3ccccc3)nc(-c3ccccc3)n2)c1 ; Brc1ccc2c(c1)c1ncccc1n2-c1cccc(-c2cc(-c3ccccc3)nc(-c3ccccc3)c2)c1]

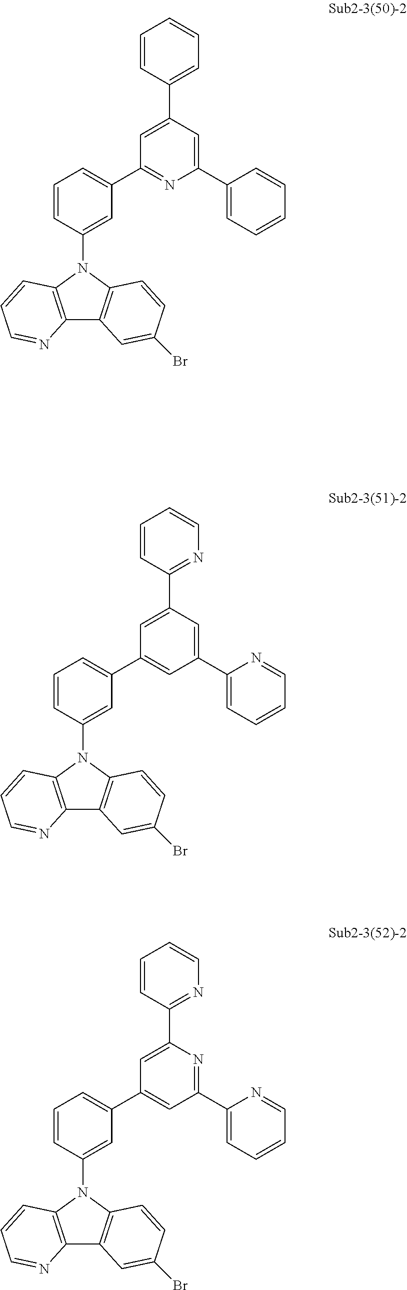 [SMILES: Brc1ccc2c(c1)c1ncccc1n2-c1cccc(-c2cc(-c3ccccc3)cc(-c3ccccc3)n2)c1 ; Brc1ccc2c(c1)c1ncccc1n2-c1cccc(-c2cc(-c3ccccn3)cc(-c3ccccn3)c2)c1 ; Brc1ccc2c(c1)c1ncccc1n2-c1cccc(-c2cc(-c3ccccn3)nc(-c3ccccn3)c2)c1]

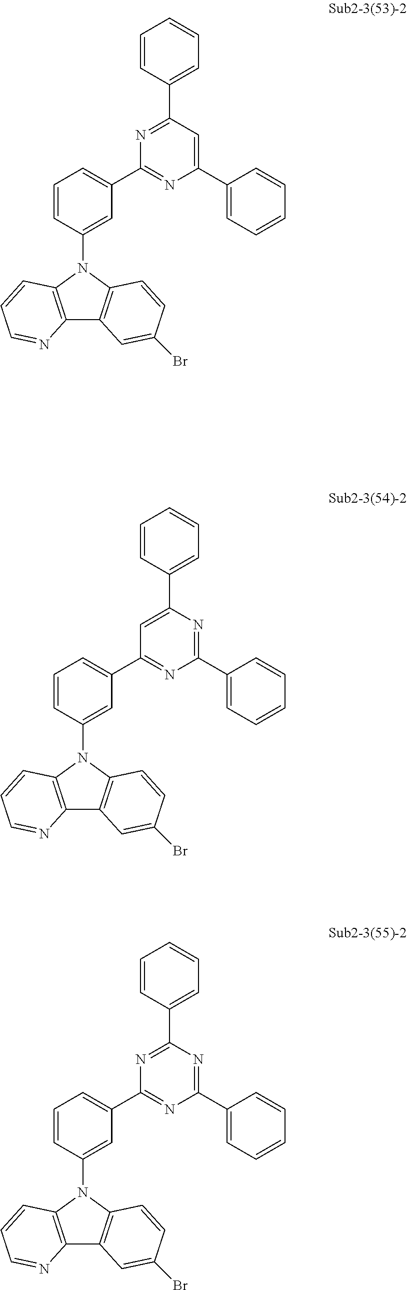 [SMILES: Brc1ccc2c(c1)c1ncccc1n2-c1cccc(-c2nc(-c3ccccc3)cc(-c3ccccc3)n2)c1 ; Brc1ccc2c(c1)c1ncccc1n2-c1cccc(-c2cc(-c3ccccc3)nc(-c3ccccc3)n2)c1 ; Brc1ccc2c(c1)c1ncccc1n2-c1cccc(-c2nc(-c3ccccc3)nc(-c3ccccc3)n2)c1]

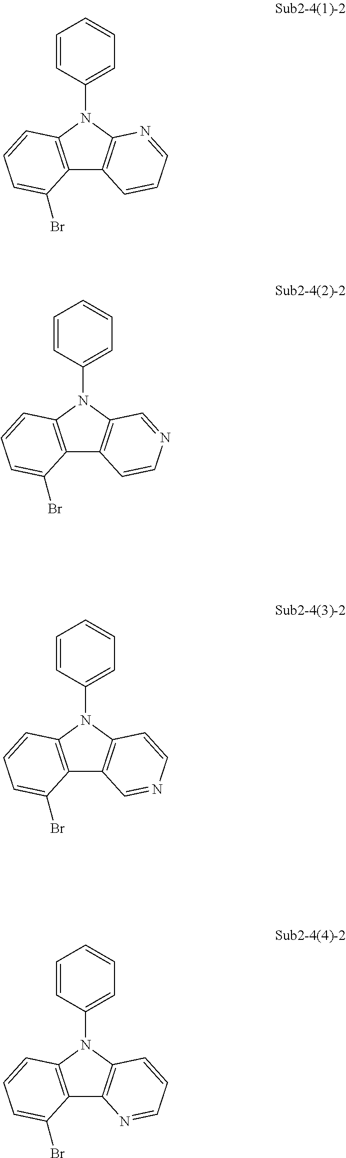 [SMILES: Brc1cccc2c1c1cccnc1n2-c1ccccc1 ; Brc1cccc2c1c1ccncc1n2-c1ccccc1 ; Brc1cccc2c1c1cnccc1n2-c1ccccc1 ; Brc1cccc2c1c1ncccc1n2-c1ccccc1]

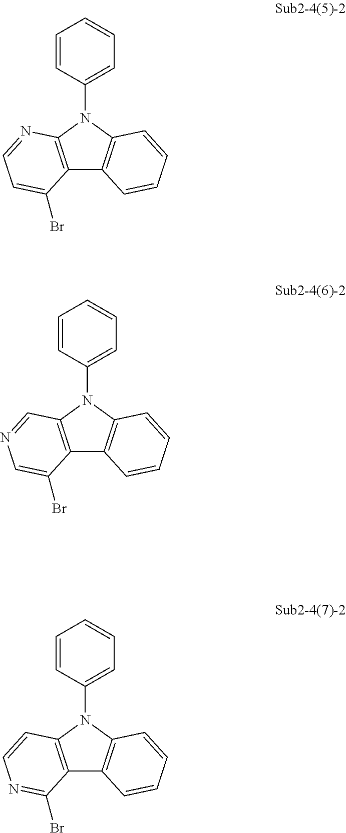 [SMILES: Brc1ccnc2c1c1ccccc1n2-c1ccccc1 ; Brc1cncc2c1c1ccccc1n2-c1ccccc1 ; Brc1nccc2c1c1ccccc1n2-c1ccccc1]

TABLE 2-3

| Compound | FD-MS | Compound | FD-MS |
|---|---|---|---|
| Sub4-2(1)-2 | m/z = 322.01(C₁₇H₁₁BrN₂ = 323.19) | Sub4-2(2)-2 | m/z = 322.01(C₁₇H₁₁BrN₂ = 323.19) |
| Sub4-2(3)-2 | m/z = 322.01(C₁₇H₁₁BrN₂ = 323.19) | Sub4-2(4)-2 | m/z = 322.01(C₁₇H₁₁BrN₂ = 323.19) |
| Sub4-2(5)-2 | m/z = 322.01(C₁₇H₁₁BrN₂ = 323.19) | Sub4-2(6)-2 | m/z = 322.01(C₁₇H₁₁BrN₂ = 323.19) |
| Sub4-2(7)-2 | m/z = 322.01(C₁₇H₁₁BrN₂ = 323.19) | Sub2-2(1)-2 | m/z = 322.01(C₁₇H₁₁BrN₂ = 323.19) |
| Sub2-2(2)-2 | m/z = 398.04(C₂₃H₁₅BrN₂ = 399.28) | Sub2-2(3)-2 | m/z = 398.04(C₂₃H₁₅BrN₂ = 399.28) |
| Sub2-2(4)-2 | m/z = 477.06(C₂₆H₁₆BrN₅ = 478.34) | Sub2-2(5)-2 | m/z = 475.07(C₂₈H₁₈BrN₃ = 476.37) |
| Sub2-2(6)-2 | m/z = 475.07(C₂₈H₁₈BrN₃ = 476.37) | Sub2-2(7)-2 | m/z = 476.06(C₂₈H₁₈BrN₃ = 477.35) |
| Sub2-2(8)-2 | m/z = 476.06(C₂₈H₁₈BrN₃ = 477.35) | Sub2-2(9)-2 | m/z = 476.06(C₂₈H₁₈BrN₃ = 477.35) |
| Sub2-2(10)-2 | m/z = 476.06(C₂₈H₁₈BrN₃ = 477.35) | Sub2-2(11)-2 | m/z = 477.06(C₂₆H₁₆BrN₅ = 478.34) |
| Sub2-2(12)-2 | m/z = 553.09(C₃₂H₂₀BrN₅ = 554.44) | Sub2-2(13)-2 | m/z = 552.09(C₃₃H₂₁BrN₄ = 553.45) |
| Sub2-2(14)-2 | m/z = 552.09(C₃₃H₂₁BrN₄ = 553.45) | Sub2-2(15)-2 | m/z = 551.10(C₃₄H₂₂BrN₃ = 552.46) |
| Sub2-2(16)-2 | m/z = 551.10(C₃₄H₂₂BrN₃ = 552.46) | Sub2-2(17)-2 | m/z = 552.09(C₃₃H₂₁BrN₄ = 553.45) |
| Sub2-2(18)-2 | m/z = 553.09(C₃₂H₂₀BrN₅ = 554.44) | Sub2-2(19)-2 | m/z = 553.09(C₃₂H₂₀BrN₅ = 554.44) |
| Sub2-2(20)-2 | m/z = 552.09(C₃₃H₂₁BrN₄ = 553.45) | Sub2-2(21)-2 | m/z = 552.09(C₃₃H₂₁BrN₄ = 553.45) |
| Sub2-2(22)-2 | m/z = 551.10(C₃₄H₂₂BrN₃ = 552.46) | Sub2-2(23)-2 | m/z = 551.10(C₃₄H₂₂BrN₃ = 552.46) |
| Sub2-2(24)-2 | m/z = 552.09(C₃₃H₂₁BrN₄ = 553.45) | Sub2-2(25)-2 | m/z = 552.09(C₃₃H₂₁BrN₄ = 553.45) |
| Sub2-2(26)-2 | m/z = 450.05(C₂₅H₁₅BrN₄ = 451.32) | Sub2-2(27)-2 | m/z = 322.01(C₁₇H₁₁BrN₂ = 323.19) |
| Sub2-2(28)-2 | m/z = 322.01(C₁₇H₁₁BrN₂ = 323.19) | Sub2-2(29)-2 | m/z = 322.01(C₁₇H₁₁BrN₂ = 323.19) |
| Sub2-2(30)-2 | m/z = 398.04(C₂₃H₁₅BrN₂ = 399.28) | Sub2-2(31)-2 | m/z = 398.04(C₂₃H₁₅BrN₂ = 399.28) |
| Sub2-2(32)-2 | m/z = 477.06(C₂₆H₁₆BrN₅ = 478.34) | Sub2-2(33)-2 | m/z = 476.06(C₂₈H₁₈BrN₃ = 477.35) |
| Sub2-2(34)-2 | m/z = 476.06(C₂₈H₁₈BrN₃ = 477.35) | Sub2-2(35)-2 | m/z = 476.06(C₂₈H₁₈BrN₃ = 477.35) |
| Sub2-2(36)-2 | m/z = 475.07(C₂₈H₁₈BrN₃ = 476.37) | Sub2-2(37)-2 | m/z = 475.07(C₂₈H₁₈BrN₃ = 476.37) |
| Sub2-2(38)-2 | m/z = 476.06(C₂₈H₁₈BrN₃ = 477.35) | Sub2-2(39)-2 | m/z = 476.06(C₂₈H₁₈BrN₃ = 477.35) |
| Sub2-2(40)-2 | m/z = 553.09(C₃₂H₂₀BrN₅ = 554.44) | Sub2-2(41)-2 | m/z = 552.09(C₃₃H₂₁BrN₄ = 553.45) |
| Sub2-2(42)-2 | m/z = 552.09(C₃₃H₂₁BrN₄ = 553.45) | Sub2-2(43)-2 | m/z = 551.10(C₃₄H₂₂BrN₃ = 552.46) |
| Sub2-2(44)-2 | m/z = 551.10(C₃₄H₂₂BrN₃ = 552.46) | Sub2-2(45)-2 | m/z = 552.09(C₃₃H₂₁BrN₄ = 553.45) |
| Sub2-2(46)-2 | m/z = 553.09(C₃₂H₂₀BrN₅ = 554.44) | Sub2-2(47)-2 | m/z = 553.09(C₃₂H₂₀BrN₅ = 554.44) |
| Sub2-2(48)-2 | m/z = 552.09(C₃₃H₂₁BrN₄ = 553.45) | Sub2-2(49)-2 | m/z = 552.09(C₃₃H₂₁BrN₄ = 553.45) |
| Sub2-2(50)-2 | m/z = 551.10(C₃₄H₂₂BrN₃ = 552.46) | Sub2-2(51)-2 | m/z = 551.10(C₃₄H₂₂BrN₃ = 552.46) |
| Sub2-2(52)-2 | m/z = 552.09(C₃₃H₂₁BrN₄ = 553.45) | Sub2-2(53)-2 | m/z = 553.09(C₃₂H₂₀BrN₅ = 554.44) |
| Sub2-2(54)-2 | m/z = 450.05(C₂₅H₁₅BrN₄ = 451.32) | Sub2-2(55)-2 | m/z = 322.01(C₁₇H₁₁BrN₂ = 323.19) |
| Sub2-2(56)-2 | m/z = 322.01(C₁₇H₁₁BrN₂ = 323.19) | Sub2-2(57)-2 | m/z = 322.01(C₁₇H₁₁BrN₂ = 323.19) |
| Sub2-3(1)-2 | m/z = 322.01(C₁₇H₁₁BrN₂ = 323.19) | Sub2-3(2)-2 | m/z = 398.04(C₂₃H₁₅BrN₂ = 399.28) |
| Sub2-3(3)-2 | m/z = 398.04(C₂₃H₁₅BrN₂ = 399.28) | Sub2-3(4)-2 | m/z = 477.06(C₂₆H₁₆BrN₅ = 478.34) |
| Sub2-3(5)-2 | m/z = 475.07(C₂₈H₁₈BrN₃ = 476.37) | Sub2-3(6)-2 | m/z = 475.07(C₂₈H₁₈BrN₃ = 476.37) |
| Sub2-3(7)-2 | m/z = 476.06(C₂₈H₁₈BrN₃ = 477.35) | Sub3-3(8)-2 | m/z = 476.06(C₂₈H₁₈BrN₃ = 477.35) |
| Sub2-3(9)-2 | m/z = 476.06(C₂₈H₁₈BrN₃ = 477.35) | Sub3-3(10)-2 | m/z = 476.06(C₂₈H₁₈BrN₃ = 477.35) |
| Sub2-3(11)-2 | m/z = 477.06(C₂₆H₁₆BrN₅ = 478.34) | Sub2-3(12)-2 | m/z = 551.10(C₃₄H₂₂BrN₃ = 552.46) |
| Sub2-3(13)-2 | m/z = 551.10(C₃₄H₂₂BrN₃ = 552.46) | Sub2-3(14)-2 | m/z = 552.09(C₃₃H₂₁BrN₄ = 553.45) |
| Sub2-3(15)-2 | m/z = 553.09(C₃₂H₂₀BrN₅ = 554.44) | Sub2-3(16)-2 | m/z = 553.09(C₃₂H₂₀BrN₅ = 554.44) |
| Sub2-3(17)-2 | m/z = 552.09(C₃₃H₂₁BrN₄ = 553.45) | Sub2-3(18)-2 | m/z = 552.09(C₃₃H₂₁BrN₄ = 553.45) |
| Sub2-3(19)-2 | m/z = 553.09(C₃₂H₂₀BrN₅ = 554.44) | Sub2-3(20)-2 | m/z = 552.09(C₃₃H₂₁BrN₄ = 553.45) |
| Sub2-3(21)-2 | m/z = 552.09(C₃₃H₂₁BrN₄ = 553.45) | Sub2-3(22)-2 | m/z = 551.10(C₃₄H₂₂BrN₃ = 552.46) |
| Sub2-3(23)-2 | m/z = 551.10(C₃₄H₂₂BrN₃ = 552.46) | Sub2-3(24)-2 | m/z = 552.09(C₃₃H₂₁BrN₄ = 553.45) |
| Sub2-3(25)-2 | m/z = 553.09(C₃₂H₂₀BrN₅ = 554.44) | Sub2-3(26)-2 | m/z = 450.05(C₂₅H₁₅BrN₄ = 451.32) |
| Sub2-3(27)-2 | m/z = 322.01(C₁₇H₁₁BrN₂ = 323.19) | Sub2-3(28)-2 | m/z = 322.01(C₁₇H₁₁BrN₂ = 323.19) |
| Sub2-3(29)-2 | m/z = 322.01(C₁₇H₁₁BrN₂ = 323.19) | Sub2-3(30)-2 | m/z = 398.04(C₂₃H₁₅BrN₂ = 399.28) |
| Sub2-3(31)-2 | m/z = 398.04(C₂₃H₁₅BrN₂ = 399.28) | Sub2-3(32)-2 | m/z = 450.05(C₂₅H₁₅BrN₄ = 451.32) |
| Sub2-3(33)-2 | m/z = 475.07(C₂₈H₁₈BrN₃ = 476.37) | Sub2-3(34)-2 | m/z = 475.07(C₂₈H₁₈BrN₃ = 476.37) |
| Sub2-3(35)-2 | m/z = 476.06(C₂₈H₁₈BrN₃ = 477.35) | Sub2-3(36)-2 | m/z = 476.06(C₂₈H₁₈BrN₃ = 477.35) |
| Sub2-3(37)-2 | m/z = 476.06(C₂₈H₁₈BrN₃ = 477.35) | Sub2-3(38)-2 | m/z = 476.06(C₂₈H₁₈BrN₃ = 477.35) |
| Sub2-3(39)-2 | m/z = 477.06(C₂₆H₁₆BrN₅ = 478.34) | Sub2-3(40)-2 | m/z = 477.06(C₂₆H₁₆BrN₅ = 478.34) |
| Sub2-3(41)-2 | m/z = 551.10(C₃₄H₂₂BrN₃ = 552.46) | Sub2-3(42)-2 | m/z = 551.10(C₃₄H₂₂BrN₃ = 552.46) |
| Sub2-3(43)-2 | m/z = 552.09(C₃₃H₂₁BrN₄ = 553.45) | Sub2-3(44)-2 | m/z = 553.09(C₃₂H₂₀BrN₅ = 554.44) |
| Sub2-3(45)-2 | m/z = 553.09(C₃₂H₂₀BrN₅ = 554.44) | Sub2-3(46)-2 | m/z = 552.09(C₃₃H₂₁BrN₄ = 553.45) |
| Sub2-3(47)-2 | m/z = 552.09(C₃₃H₂₁BrN₄ = 553.45) | Sub2-3(48)-2 | m/z = 553.09(C₃₂H₂₀BrN₅ = 554.44) |
| Sub2-3(49)-2 | m/z = 551.10(C₃₄H₂₂BrN₃ = 552.46) | Sub2-3(50)-2 | m/z = 551.10(C₃₄H₂₂BrN₃ = 552.46) |
| Sub2-3(51)-2 | m/z = 552.09(C₃₃H₂₁BrN₄ = 553.45) | Sub2-3(52)-2 | m/z = 553.09(C₃₂H₂₀BrN₅ = 554.44) |
| Sub2-3(53)-2 | m/z = 553.09(C₃₂H₂₀BrN₅ = 554.44) | Sub2-3(54)-2 | m/z = 552.09(C₃₃H₂₁BrN₄ = 553.45) |
| Sub2-3(55)-2 | m/z = 553.09(C₃₂H₂₀BrN₅ = 554.44) | Sub4-2(1)-2 | m/z = 322.01(C₁₇H₁₁BrN₂ = 323.19) |
| Sub4-2(2)-2 | m/z = 322.01(C₁₇H₁₁BrN₂ = 323.19) | Sub4-2(3)-2 | m/z = 322.01(C₁₇H₁₁BrN₂ = 323.19) |
| Sub4-2(4)-2 | m/z = 322.01(C₁₇H₁₁BrN₂ = 323.19) | Sub4-2(5)-2 | m/z = 322.01(C₁₇H₁₁BrN₂ = 323.19) |
| Sub4-2(6)-2 | m/z = 322.01(C₁₇H₁₁BrN₂ = 323.19) | Sub4-2(7)-2 | m/z = 322.01(C₁₇H₁₁BrN₂ = 323.19) |

### III. Synthesis Example of Final Products

In a round-bottom flask, compound Sub 1-2 (1 eq) was added, and then compound Sub 2-2 (1.1 eq), Pd(PPh₃)₄ (0.03-0.05 eq.), NaOH (3 eq), THF (3 mL/1 mmol), and water (1.5 mL/1 mmol) were added. Thereafter, the mixture was heated under reflux at 80-90° C. Upon completion of the reaction, the reaction product was diluted with distilled water at room temperature, followed by extraction with methylene chloride and water. The organic layer was dried over MgSO₄ and concentrated, and then the generated compound was subjected to silica gel chromatography and recrystallization to give a product.

### Synthesis Example of Compound 1-1-2

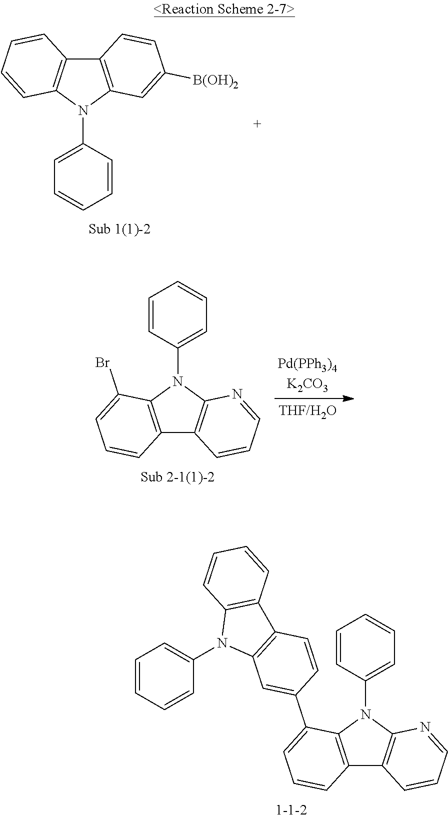 [SMILES: OB(O)c1ccc2c3ccccc3n(-c3ccccc3)c2c1 ; Brc1cccc2c3cccnc3n(-c3ccccc3)c12 ; c1ccc(-n2c3ccccc3c3ccc(-c4cccc5c6cccnc6n(-c6ccccc6)c45)cc32)cc1]

In a round-bottom flask, (9-phenyl-9H-carbazol-1-yl)boronic acid (5.7 g, 20 mmol) was added, and then 8-bromo-9-phenyl-9H-pyrido[2,3-b]indole (12.2 g, 22 mmol), Pd(PPh₃)₄ (0.5 g, 0.6 mmol), K₂CO₃ (8.3 g, 60 mmol), THF (60 mL), and water (30 mL) were added. Thereafter, the mixture was heated under reflux at 80-90□. Upon completion of the reaction, the reaction product was diluted with distilled water at room temperature, followed by extraction with methylene chloride and water. The organic layer was dried over MgSO₄ and concentrated, and then the generated compound was subjected to silica gel chromatography and recrystallization to give a product 5.8 g (yield: 60%).

### 2. Synthesis Example of Compound 2-38-2

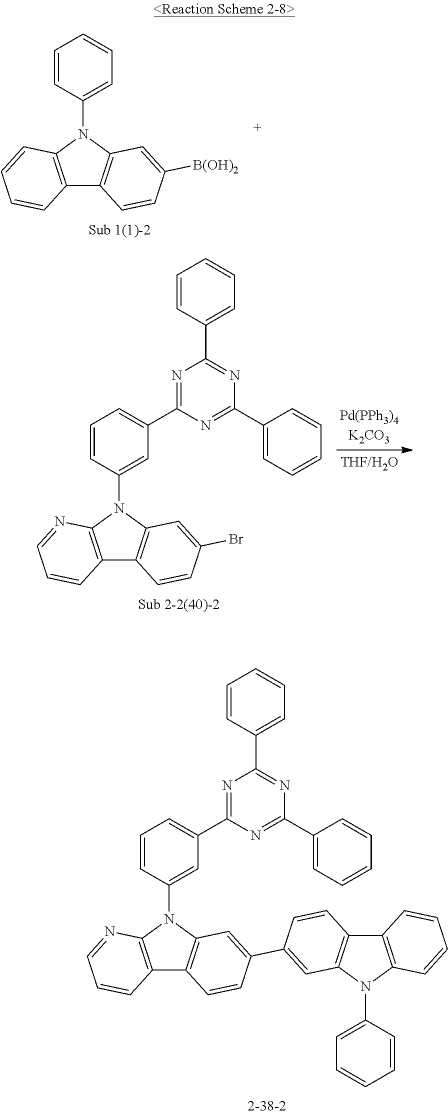 [SMILES: OB(O)c1ccc2c3ccccc3n(-c3ccccc3)c2c1 ; Brc1ccc2c3cccnc3n(-c3cccc(-c4nc(-c5ccccc5)nc(-c5ccccc5)n4)c3)c2c1 ; c1ccc(-c2nc(-c3ccccc3)nc(-c3cccc(-n4c5cc(-c6ccc7c8ccccc8n(-c8ccccc8)c7c6)ccc5c5cccnc54)c3)n2)cc1]

In a round-bottom flask, (9-phenyl-9H-carbazol-1-yl)boronic acid (5.7 g, 20 mmol) was added, and then 7-bromo-9-(3-(4,6-diphenyl-1,3,5-triazin-2-yl)phenyl)-9H-pyrido[2,3-b]indole (12.2 g, 22 mmol), Pd(PPh₃)₄ (0.5 g, 0.6 mmol), K₂CO₃ (8.3 g, 60 mmol), THF (60 mL), and water (30 mL) were added. Thereafter, the mixture was heated under reflux at 80-90□. Upon completion of the reaction, the reaction product was diluted with distilled water at room temperature, followed by extraction with methylene chloride and water. The organic layer was dried over MgSO₄ and concentrated, and then the thus generated compound was subjected to silica gel column chromatography and recrystallization to give a product 8.3 g (yield: 58%).

### 3. Synthesis Example of Compound 2-70-2

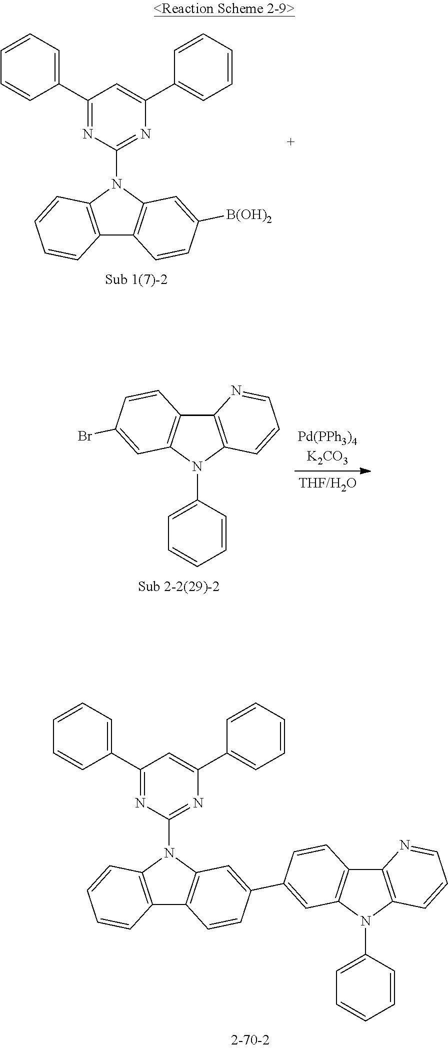 [SMILES: OB(O)c1ccc2c3ccccc3n(-c3nc(-c4ccccc4)cc(-c4ccccc4)n3)c2c1 ; Brc1ccc2c3ncccc3n(-c3ccccc3)c2c1 ; c1ccc(-c2cc(-c3ccccc3)nc(-n3c4ccccc4c4ccc(-c5ccc6c7ncccc7n(-c7ccccc7)c6c5)cc43)n2)cc1]

In a round-bottom flask, (9-(4,6-diphenylpyrimidin-2-yl)-9H-carbazol-1-yl)boronic acid (8.8 g, 20 mmol) was added, and then 7-bromo-9-(3-(4,6-diphenyl-1,3,5-triazin-2-yl)phenyl)-9H-pyrido[2,3-b]indole (12.2 g, 22 mmol), Pd(PPh₃)₄ (0.5 g, 0.6 mmol), K₂CO₃ (8.3 g, 60 mmol), THF (60 mL), water (30 mL) were added. Thereafter, the mixture was heated under reflux at 80-90° C. Upon completion of the reaction, the reaction product was diluted with distilled water at room temperature, followed by extraction with methylene chloride and water. The organic layer was dried over MgSO₄ and concentrated, and then the thus generated compound was subjected to silica gel column chromatography and recrystallization to give a product 8.3 g (yield: 65%).

### 4. Synthesis Example of Compound 3-10-2

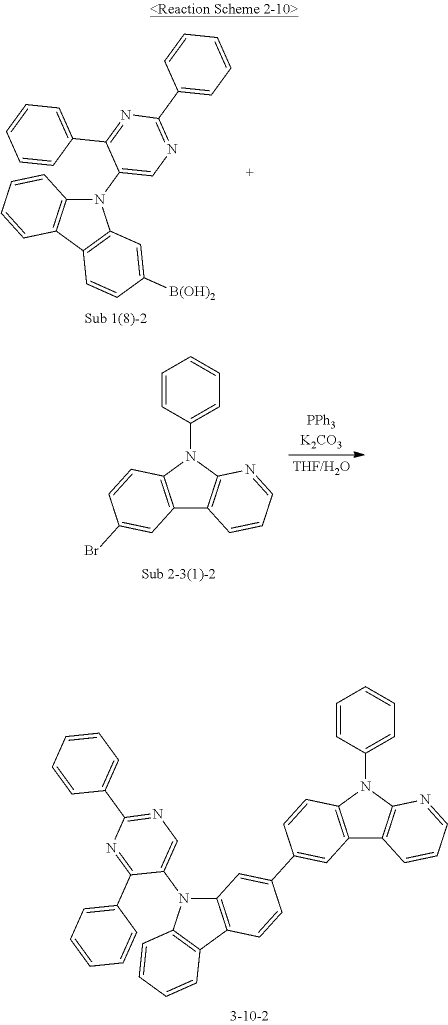 [SMILES: OB(O)c1ccc2c3ccccc3n(-c3cnc(-c4ccccc4)nc3-c3ccccc3)c2c1 ; Brc1ccc2c(c1)c1cccnc1n2-c1ccccc1 ; c1ccc(-c2ncc(-n3c4ccccc4c4ccc(-c5ccc6c(c5)c5cccnc5n6-c5ccccc5)cc43)c(-c3ccccc3)n2)cc1]

In a round-bottom flask, (9-(2,4-diphenylpyrimidin-5-yl)-9H-carbazol-1-yl)boronic acid (8.8 g, 20 mmol) was added, and then 6-bromo-9-phenyl-9H-pyrido[2,3-b]indole (7.1 g, 22 mmol), Pd(PPh₃)₄ (0.5 g, 0.6 mmol), K₂CO₃ (8.3 g, 60 mmol), THF (60 mL), and water (30 mL) were added. Thereafter, the mixture was heated under reflux at 80-90° C. Upon completion of the reaction, the reaction product was diluted with distilled water at room temperature, followed by extraction with methylene chloride and water. The organic layer was dried over MgSO₄ and concentrated, and then the thus generated compound was subjected to silica gel column chromatography and recrystallization to give a product 7.7 g (yield: 60%).

### 5. Synthesis Example of Compound 3-68-2

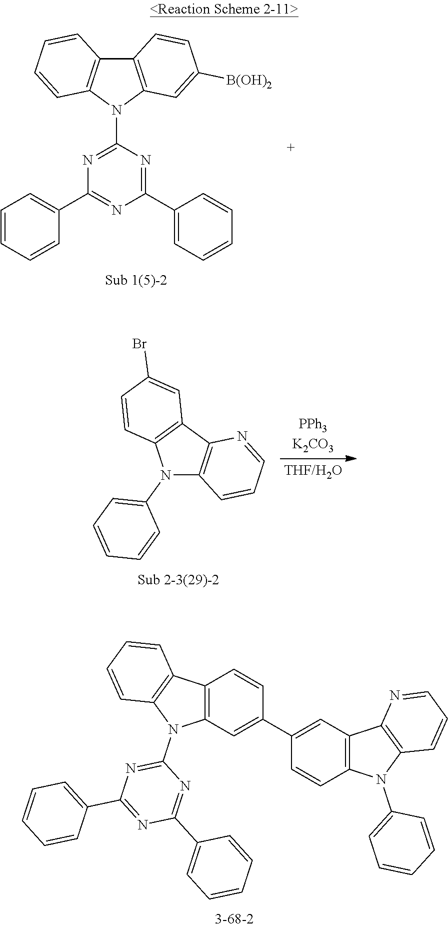 [SMILES: OB(O)c1ccc2c3ccccc3n(-c3nc(-c4ccccc4)nc(-c4ccccc4)n3)c2c1 ; Brc1ccc2c(c1)c1ncccc1n2-c1ccccc1 ; c1ccc(-c2nc(-c3ccccc3)nc(-n3c4ccccc4c4ccc(-c5ccc6c(c5)c5ncccc5n6-c5ccccc5)cc43)n2)cc1]

In a round-bottom flask, (9-(4,6-diphenyl-1,3,5-triazin-2-yl)-9H-carbazol-1-yl)boronic acid (8.8 g, 20 mmol) was added, and then 8-bromo-5-phenyl-5H-pyrido[3,2-b]indole (7.1 g, 22 mmol), Pd(PPh₃)₄ (0.5 g, 0.6 mmol), K₂CO₃ (8.3 g, 60 mmol), THF (60 mL), and water (30 mL) were added.

Thereafter, the mixture was heated under reflux at 80-90° C.
Upon completion of the reaction, the reaction product was diluted with distilled water at room temperature, followed by extraction with methylene chloride and water. The organic layer was dried over MgSO₄ and concentrated, and then the thus generated compound was subjected to silica gel column chromatography and recrystallization to give a product 7.0 g (yield: 54%).

### 6. Synthesis Example of Compound 3-76-2

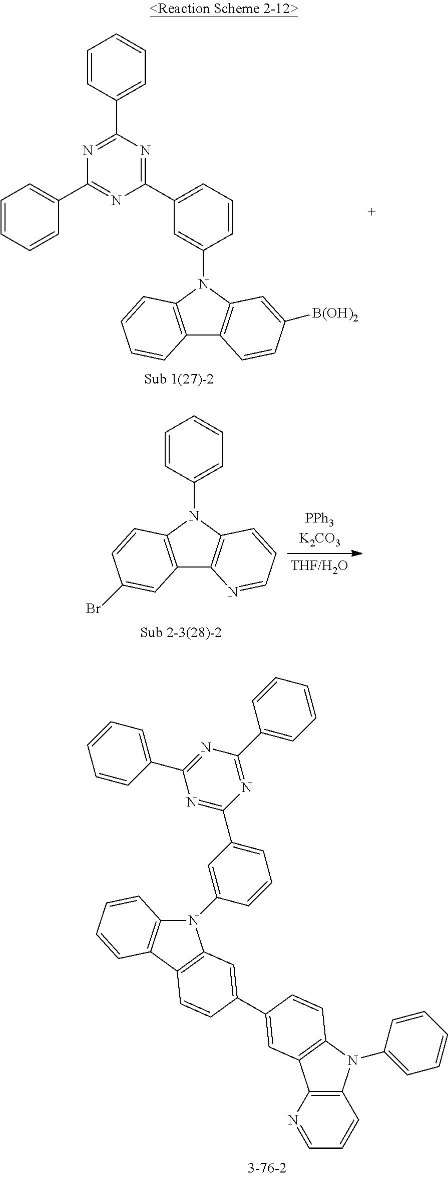 [SMILES: OB(O)c1ccc2c3ccccc3n(-c3cccc(-c4nc(-c5ccccc5)nc(-c5ccccc5)n4)c3)c2c1 ; Brc1ccc2c(c1)c1ncccc1n2-c1ccccc1 ; c1ccc(-c2nc(-c3ccccc3)nc(-c3cccc(-n4c5ccccc5c5ccc(-c6ccc7c(c6)c6ncccc6n7-c6ccccc6)cc54)c3)n2)cc1]

In a round-bottom flask, (9-(3-(4,6-diphenyl-1,3,5-triazin-2-yl)phenyl)-9H-carbazol-1-yl)boronic acid (10.4 g, 20 mmol) was added, and then 8-bromo-5-phenyl-5H-pyrido[3,2-b]indole (7.1 g, 22 mmol), Pd(PPh₃)₄ (0.5 g, 0.6 mmol), K₂CO₃ (8.3 g, 60 mmol), THF (60 mL), and water (30 mL) were added.

Thereafter, the mixture was heated under reflux at 80-90° C.
Upon completion of the reaction, the reaction product was diluted with distilled water at room temperature, followed by extraction with methylene chloride and water. The organic layer was dried over MgSO₄ and concentrated, and then the thus generated compound was subjected to silica gel column chromatography and recrystallization to give a product 9.7 g (yield: 68%).

### 7. Synthesis Example of Compound 4-23-2

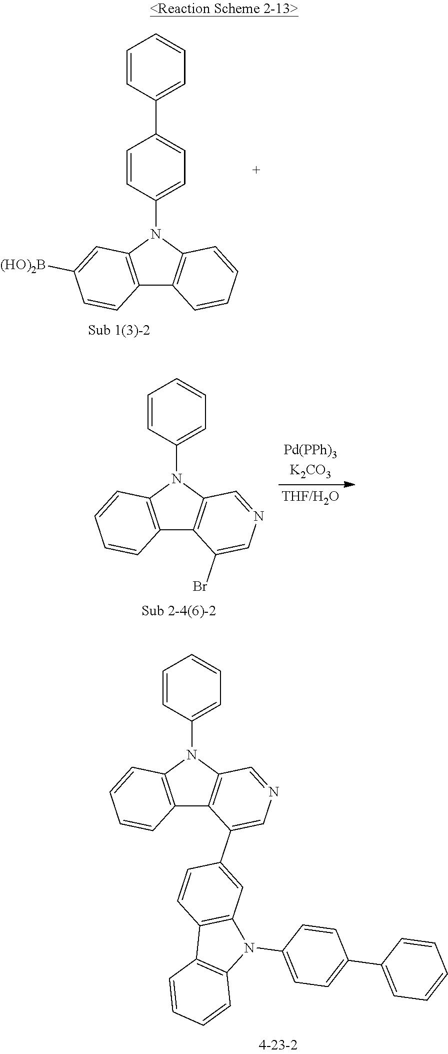 [SMILES: OB(O)c1ccc2c3ccccc3n(-c3ccc(-c4ccccc4)cc3)c2c1 ; Brc1cncc2c1c1ccccc1n2-c1ccccc1 ; c1ccc(-c2ccc(-n3c4ccccc4c4ccc(-c5cncc6c5c5ccccc5n6-c5ccccc5)cc43)cc2)cc1]

In a round-bottom flask, (9-([1,1′-biphenyl]-4-yl)-9H-carbazol-1-yl)boronic acid (7.2 g, 20 mmol) was added, and then 4-bromo-9-phenyl-9H-pyrido[3,4-b]indole (7.1 g, 22 mmol), Pd(PPh₃)₄ (0.5 g, 0.6 mmol), K₂CO₃ (8.3 g, 60 mmol), THF (60 mL), and water (30 mL) were added. Thereafter, the mixture was heated under reflex at 80-90° C. Upon completion of the reaction, the reaction product was diluted with distilled water at room temperature, followed by extraction with methylene chloride and water. The organic layer was dried over MgSO₄ and concentrated, and then the thus generated compound was subjected to silica gel column chromatography and recrystallization to give a product 7.8 g (yield: 69%).

Meanwhile, FD-MS values of compounds 1-1-2 to 1-28-2, 2-1-2 to 2-128-2, 3-1-2 to 3-128-2, 4-1-2 to 4-28-2, and 5-1-2 to 5-4-2 of the present invention prepared by the above synthesis examples are shown as in table 1-4 below.

TABLE 2-4

| Compound | FD-MS | Compound | FD-MS |
|---|---|---|---|
| 1-1-2 | m/z = 485.19 (C₃₅H₂₅N₃ = 485.58) | 1-2-2 | m/z = 535.20 (C₃₉H₂₅N₃ = 535.64) |
| 1-3-2 | m/z = 561.22 (C₄₁H₂₇N₃ = 561.67) | 1-4-2 | m/z = 640.24 (C₄₄H₂₈N₆ = 640.73) |
| 1-5-2 | m/z = 485.19 (C₃₅H₂₃N₃ = 485.58) | 1-6-2 | m/z = 535.20 (C₃₉H₂₅N₃ = 535.64) |
| 1-7-2 | m/z = 561.22 (C₄₁H₂₇N₃ = 561.67) | 1-8-2 | m/z = 640.24 (C₄₄H₂₈N₆ = 640.73) |
| 1-9-2 | m/z = 485.19 (C₃₅H₂₃N₃ = 485.58) | 1-20-2 | m/z = 535.20 (C₃₉H₂₅N₃ = 535.64) |
| 1-11-2 | m/z = 561.22 (C₄₁H₂₇N₃ = 561.67) | 1-22-2 | m/z = 640.24 (C₄₄H₂₈N₆ = 640.73) |
| 1-13-2 | m/z = 485.19 (C₃₅H₂₃N₃ = 485.58) | 1-24-2 | m/z = 535.20 (C₃₉H₂₅N₃ = 535.64) |
| 1-15-2 | m/z = 561.22 (C₄₁H₂₇N₃ = 561.67) | 1-26-2 | m/z = 640.24 (C₄₄H₂₈N₆ = 640.73) |
| 1-17-2 | m/z = 485.19 (C₃₅H₂₃N₃ = 485.58) | 1-28-2 | m/z = 535.20 (C₃₉H₂₅N₃ = 535.64) |
| 1-19-2 | m/z = 561.22 (C₄₁H₂₇N₃ = 561.67) | 1-20-2 | m/z = 640.24 (C₄₄H₂₈N₆ = 640.73) |
| 1-21-2 | m/z = 485.19 (C₃₅H₂₃N₃ = 485.58) | 1-22-2 | m/z = 535.20 (C₃₉H₂₅N₃ = 535.64) |
| 1-23-2 | m/z = 561.22 (C₄₁H₂₇N₃ = 561.67) | 1-24-2 | m/z = 640.24 (C₄₄H₂₈N₆ = 640.73) |
| 1-25-2 | m/z = 485.19 (C₃₅H₂₃N₃ = 485.58) | 1-26-2 | m/z = 535.20 (C₃₉H₂₅N₃ = 535.64) |
| 1-27-2 | m/z = 561.22 (C₄₁H₂₇N₃ = 561.67) | 1-28-2 | m/z = 640.24 (C₄₄H₂₈N₆ = 640.73) |
| 2-1-2 | m/z = 485.19 (C₃₅H₂₃N₃ = 485.58) | 2-2-2 | m/z = 561.22 (C₄₁H₂₇N₃ = 561.67) |
| 2-3-2 | m/z = 561.22 (C₄₁H₂₇N₃ = 561.67) | 2-4-2 | m/z = 637.25 (C₄₇H₃₁N₃ = 637.77) |
| 2-5-2 | m/z = 637.25 (C₄₇H₃₁N₃ = 637.77) | 2-6-2 | m/z = 637.25 (C₄₇H₃₁N₃ = 637.77) |
| 2-7-2 | m/z = 637.25 (C₄₇H₃₁N₃ = 637.77) | 2-8-2 | m/z = 639.24 (C₄₅H₂₉N₅ = 639.75) |
| 2-9-2 | m/z = 639.24 (C₄₅H₂₉N₅ = 639.75) | 2-20-2 | m/z = 639.24 (C₄₅H₂₉N₅ = 639.75) |
| 2-11-2 | m/z = 638.25 (C₄₆H₃₀N₄ = 638.76) | 2-22-2 | m/z = 638.25 (C₄₆H₃₀N₄ = 638.76) |
| 2-13-2 | m/z = 639.24 (C₄₅H₂₉N₅ = 639.75) | 2-24-2 | m/z = 640.24 (C₄₄H₂₈N₆ = 640.73) |
| 2-15-2 | m/z = 716.27 (C₅₀H₃₂N₆ = 716.83) | 2-26-2 | m/z = 715.27 (C₅₁H₃₃N₅ = 715.84) |
| 2-17-2 | m/z = 715.27 (C₅₁H₃₃N₅ = 715.84) | 2-28-2 | m/z = 714.28 (C₅₂H₃₄N₄ = 714.85) |
| 2-19-2 | m/z = 714.28 (C₅₂H₃₄N₄ = 714.85) | 2-20-2 | m/z = 715.27 (C₅₁H₃₃N₅ = 715.84) |
| 2-21-2 | m/z = 716.27 (C₅₀H₃₂N₆ = 716.83) | 2-22-2 | m/z = 716.27 (C₅₀H₃₂N₆ = 716.83) |
| 2-23-2 | m/z = 715.27 (C₅₁H₃₃N₅ = 715.84) | 2-24-2 | m/z = 715.27 (C₅₁H₃₃N₅ = 715.84) |
| 2-25-2 | m/z = 714.28 (C₅₂H₃₄N₄ = 714.85) | 2-26-2 | m/z = 714.28 (C₅₂H₃₄N₄ = 714.85) |
| 2-27-2 | m/z = 715.27 (C₅₁H₃₃N₅ = 715.84) | 2-28-2 | m/z = 716.27 (C₅₀H₃₂N₆ = 716.83) |
| 2-29-2 | m/z = 613.23 (C₄₃H₂₇N₅ = 613.71) | 2-30-2 | m/z = 640.24 (C₄₄H₂₈N₆ = 640.73) |
| 2-31-2 | m/z = 639.24 (C₄₅H₂₉N₅ = 639.75) | 2-32-2 | m/z = 639.24 (C₄₅H₂₉N₅ = 639.75) |
| 2-33-2 | m/z = 639.24 (C₄₅H₂₉N₅ = 639.75) | 2-34-2 | m/z = 638.25 (C₄₆H₃₀N₄ = 638.76) |
| 2-35-2 | m/z = 638.25 (C₄₆H₃₀N₄ = 638.76) | 2-36-2 | m/z = 639.24 (C₄₅H₂₉N₅ = 639.75) |
| 2-37-2 | m/z = 640.24 (C₄₄H₂₈N₆ = 640.73) | 2-38-2 | m/z = 716.27 (C₅₀H₃₂N₆ = 716.83) |
| 2-39-2 | m/z = 715.27 (C₅₁H₃₃N₅ = 715.84) | 2-40-2 | m/z = 715.27 (C₅₁H₃₃N₅ = 715.84) |
| 2-41-2 | m/z = 714.28 (C₅₂H₃₄N₄ = 714.85) | 2-42-2 | m/z = 714.28 (C₅₂H₃₄N₄ = 714.85) |
| 2-43-2 | m/z = 715.27 (C₅₁H₃₃N₅ = 715.84) | 2-44-2 | m/z = 716.27 (C₅₀H₃₂N₆ = 716.83) |
| 2-45-2 | m/z = 716.27 (C₅₀H₃₂N₆ = 716.83) | 2-46-2 | m/z = 715.27 (C₅₁H₃₃N₅ = 715.84) |
| 2-47-2 | m/z = 715.27 (C₅₁H₃₃N₅ = 715.84) | 2-48-2 | m/z = 714.28 (C₅₂H₃₄N₄ = 714.85) |
| 2-49-2 | m/z = 714.28 (C₅₂H₃₄N₄ = 714.85) | 2-50-2 | m/z = 715.27 (C₅₁H₃₃N₅ = 715.84) |
| 2-51-2 | m/z = 716.27 (C₅₀H₃₂N₆ = 716.83) | 2-52-2 | m/z = 613.23 (C₄₃H₂₇N₅ = 613.71) |
| 2-53-2 | m/z = 485.19 (C₃₅H₂₃N₃ = 485.58) | 2-54-2 | m/z = 535.20 (C₃₉H₂₅N₃ = 535.64) |
| 2-55-2 | m/z = 561.22 (C₄₁H₂₇N₃ = 561.67) | 2-56-2 | m/z = 640.24 (C₄₄H₂₈N₆ = 640.73) |
| 2-57-2 | m/z = 485.19 (C₃₅H₂₃N₃ = 485.58) | 2-58-2 | m/z = 535.20 (C₃₉H₂₅N₃ = 535.64) |
| 2-59-2 | m/z = 561.22 (C₄₁H₂₇N₃ = 561.67) | 2-60-2 | m/z = 640.24 (C₄₄H₂₈N₆ = 640.73) |
| 2-61-2 | m/z = 485.19 (C₃₅H₂₃N₃ = 485.58) | 2-62-2 | m/z = 561.22 (C₄₁H₂₇N₃ = 561.67) |
| 2-63-2 | m/z = 561.22 (C₄₁H₂₇N₃ = 561.67) | 2-64-2 | m/z = 637.25 (C₄₇H₃₁N₃ = 637.77) |
| 2-65-2 | m/z = 637.25 (C₄₇H₃₁N₃ = 637.77) | 2-66-2 | m/z = 637.25 (C₄₇H₃₁N₃ = 637.77) |
| 2-67-2 | m/z = 637.25 (C₄₇H₃₁N₃ = 637.77) | 2-68-2 | m/z = 640.24 (C₄₄H₂₈N₆ = 640.73) |
| 2-69-2 | m/z = 639.24 (C₄₅H₂₉N₅ = 639.75) | 2-70-2 | m/z = 639.24 (C₄₅H₂₉N₅ = 639.75) |
| 2-71-2 | m/z = 639.24 (C₄₅H₂₉N₅ = 639.75) | 2-72-2 | m/z = 638.25 (C₄₆H₃₀N₄ = 638.76) |
| 2-73-2 | m/z = 638.25 (C₄₆H₃₀N₄ = 638.76) | 2-74-2 | m/z = 639.24 (C₄₅H₂₉N₅ = 639.75) |
| 2-75-2 | m/z = 640.24 (C₄₄H₂₈N₆ = 640.73) | 2-76-2 | m/z = 716.27 (C₅₀H₃₂N₆ = 716.83) |
| 2-77-2 | m/z = 715.27 (C₅₁H₃₃N₅ = 715.84) | 2-78-2 | m/z = 715.27 (C₅₁H₃₃N₅ = 715.84) |
| 2-79-2 | m/z = 714.28 (C₅₂H₃₄N₄ = 714.85) | 2-80-2 | m/z = 714.28 (C₅₂H₃₄N₄ = 714.85) |
| 2-81-2 | m/z = 715.27 (C₅₁H₃₃N₅ = 715.84) | 2-82-2 | m/z = 716.27 (C₅₀H₃₂N₆ = 716.83) |
| 2-83-2 | m/z = 716.27 (C₅₀H₃₂N₆ = 716.83) | 2-84-2 | m/z = 715.27 (C₅₁H₃₃N₅ = 715.84) |
| 2-85-2 | m/z = 715.27 (C₅₁H₃₃N₅ = 715.84) | 2-86-2 | m/z = 714.28 (C₅₂H₃₄N₄ = 714.85) |
| 2-87-2 | m/z = 714.28 (C₅₂H₃₄N₄ = 714.85) | 2-88-2 | m/z = 715.27 (C₅₁H₃₃N₅ = 715.84) |
| 2-89-2 | m/z = 716.27 (C₅₀H₃₂N₆ = 716.83) | 2-90-2 | m/z = 613.23 (C₄₃H₂₇N₅ = 613.71) |
| 2-91-2 | m/z = 640.24 (C₄₄H₂₈N₆ = 640.73) | 2-92-2 | m/z = 639.24 (C₄₅H₂₉N₅ = 639.75) |
| 2-93-2 | m/z = 639.24 (C₄₅H₂₉N₅ = 639.75) | 2-94-2 | m/z = 639.24 (C₄₅H₂₉N₅ = 639.75) |
| 2-95-2 | m/z = 638.25 (C₄₆H₃₀N₄ = 638.76) | 2-96-2 | m/z = 638.25 (C₄₆H₃₀N₄ = 638.76) |
| 2-97-2 | m/z = 639.24 (C₄₅H₂₉N₅ = 639.75) | 2-98-2 | m/z = 640.24 (C₄₄H₂₈N₆ = 640.73) |
| 2-99-2 | m/z = 716.27 (C₅₀H₃₂N₆ = 716.83) | 2-100-2 | m/z = 715.27 (C₅₁H₃₃N₅ = 715.84) |
| 2-101-2 | m/z = 715.27 (C₅₁H₃₃N₅ = 715.84) | 2-102-2 | m/z = 714.28 (C₅₂H₃₄N₄ = 714.85) |
| 2-103-2 | m/z = 714.28 (C₅₂H₃₄N₄ = 714.85) | 2-104-2 | m/z = 715.27 (C₅₁H₃₃N₅ = 715.84) |
| 2-105-2 | m/z = 716.27 (C₅₀H₃₂N₆ = 716.83) | 2-106-2 | m/z = 716.27 (C₅₀H₃₂N₆ = 716.83) |
| 2-107-2 | m/z = 715.27 (C₅₁H₃₃N₅ = 715.84) | 2-108-2 | m/z = 715.27 (C₅₁H₃₃N₅ = 715.84) |
| 2-109-2 | m/z = 714.28 (C₅₂H₃₄N₄ = 714.85) | 2-110-2 | m/z = 714.28 (C₅₂H₃₄N₄ = 714.85) |
| 2-111-2 | m/z = 715.27 (C₅₁H₃₃N₅ = 715.84) | 2-112-2 | m/z = 716.27 (C₅₀H₃₂N₆ = 716.83) |
| 2-113-2 | m/z = 613.23 (C₄₃H₂₇N₅ = 613.71) | 2-114-2 | m/z = 485.19 (C₃₅H₂₃N₃ = 485.58) |
| 2-115-2 | m/z = 535.20 (C₃₉H₂₅N₃ = 535.64) | 2-116-2 | m/z = 561.22 (C₄₁H₂₇N₃ = 561.67) |
| 2-117-2 | m/z = 640.24 (C₄₄H₂₈N₆ = 640.73) | 2-118-2 | m/z = 485.19 (C₃₅H₂₃N₃ = 485.58) |
| 2-119-2 | m/z = 535.20 (C₃₉H₂₅N₃ = 535.64) | 2-120-2 | m/z = 561.22 (C₄₁H₂₇N₃ = 561.67) |
| 2-121-2 | m/z = 640.24 (C₄₄H₂₈N₆ = 640.73) | 2-122-2 | m/z = 485.19 (C₃₅H₂₃N₃ = 485.58) |
| 2-123-2 | m/z = 535.20 (C₃₉H₂₅N₃ = 535.64) | 2-124-2 | m/z = 561.22 (C₄₁H₂₇N₃ = 561.67) |
| 2-125-2 | m/z = 640.24 (C₄₄H₂₈N₆ = 640.73) | 2-126-2 | m/z = 640.24 (C₄₄H₂₈N₆ = 640.73) |
| 2-127-2 | m/z = 535.20 (C₃₉H₂₅N₃ = 535.64) | 2-128-2 | m/z = 535.20 (C₃₉H₂₅N₃ = 535.64) |
| 3-1-2 | m/z = 485.19 (C₃₅H₂₃N₃ = 485.58) | 3-2-2 | m/z = 561.22 (C₄₁H₂₇N₃ = 561.67) |
| 3-3-2 | m/z = 561.22 (C₄₁H₂₇N₃ = 561.67) | 3-4-2 | m/z = 637.25 (C₄₇H₃₁N₃ = 637.77) |
| 3-5-2 | m/z = 637.25 (C₄₇H₃₁N₃ = 637.77) | 3-6-2 | m/z = 637.25 (C₄₇H₃₁N₃ = 637.77) |
| 3-7-2 | m/z = 637.25 (C₄₇H₃₁N₃ = 637.77) | 3-8-2 | m/z = 639.24 (C₄₅H₂₉N₅ = 639.75) |
| 3-9-2 | m/z = 639.24 (C₄₅H₂₉N₅ = 639.75) | 3-20-2 | m/z = 639.24 (C₄₅H₂₉N₅ = 639.75) |
| 3-11-2 | m/z = 638.25 (C₄₆H₃₀N₄ = 638.76) | 3-22-2 | m/z = 638.25 (C₄₆H₃₀N₄ = 638.76) |
| 3-13-2 | m/z = 639.24 (C₄₅H₂₉N₅ = 639.75) | 3-24-2 | m/z = 640.24 (C₄₄H₂₈N₆ = 640.73) |
| 3-15-2 | m/z = 716.27 (C₅₀H₃₂N₆ = 716.83) | 3-26-2 | m/z = 715.27 (C₅₁H₃₃N₅ = 715.84) |
| 3-17-2 | m/z = 715.27 (C₅₁H₃₃N₅ = 715.84) | 3-28-2 | m/z = 714.28 (C₅₂H₃₄N₄ = 714.85) |
| 3-19-2 | m/z = 714.28 (C₅₂H₃₄N₄ = 714.85) | 3-20-2 | m/z = 715.27 (C₅₁H₃₃N₅ = 715.84) |
| 3-21-2 | m/z = 716.27 (C₅₀H₃₂N₆ = 716.83) | 3-22-2 | m/z = 716.27 (C₅₀H₃₂N₆ = 716.83) |
| 3-23-2 | m/z = 715.27 (C₅₁H₃₃N₅ = 715.84) | 3-24-2 | m/z = 715.27 (C₅₁H₃₃N₅ = 715.84) |
| 3-25-2 | m/z = 714.28 (C₅₂H₃₄N₄ = 714.85) | 3-26-2 | m/z = 714.28 (C₅₂H₃₄N₄ = 714.85) |
| 3-27-2 | m/z = 715.27 (C₅₁H₃₃N₅ = 715.84) | 3-28-2 | m/z = 716.27 (C₅₀H₃₂N₆ = 716.83) |
| 3-29-2 | m/z = 613.23 (C₄₃H₂₇N₅ = 613.71) | 3-30-2 | m/z = 640.24 (C₄₄H₂₈N₆ = 640.73) |
| 3-31-2 | m/z = 639.24 (C₄₅H₂₉N₅ = 639.75) | 3-32-2 | m/z = 639.24 (C₄₅H₂₉N₅ = 639.75) |
| 3-33-2 | m/z = 639.24 (C₄₅H₂₉N₅ = 639.75) | 3-34-2 | m/z = 638.25 (C₄₆H₃₀N₄ = 638.76) |
| 3-35-2 | m/z = 638.25 (C₄₆H₃₀N₄ = 638.76) | 3-36-2 | m/z = 639.24 (C₄₅H₂₉N₅ = 639.75) |
| 3-37-2 | m/z = 640.24 (C₄₄H₂₈N₆ = 640.73) | 3-38-2 | m/z = 716.27 (C₅₀H₃₂N₆ = 716.83) |
| 3-39-2 | m/z = 715.27 (C₅₁H₃₃N₅ = 715.84) | 3-40-2 | m/z = 715.27 (C₅₁H₃₃N₅ = 715.84) |
| 3-41-2 | m/z = 714.28 (C₅₂H₃₄N₄ = 714.85) | 3-42-2 | m/z = 714.28 (C₅₂H₃₄N₄ = 714.85) |
| 3-43-2 | m/z = 715.27 (C₅₁H₃₃N₅ = 715.84) | 3-44-2 | m/z = 716.27 (C₅₀H₃₂N₆ = 716.83) |
| 3-45-2 | m/z = 716.27 (C₅₀H₃₂N₆ = 716.83) | 3-46-2 | m/z = 715.27 (C₅₁H₃₃N₅ = 715.84) |
| 3-47-2 | m/z = 715.27 (C₅₁H₃₃N₅ = 715.84) | 3-48-2 | m/z = 714.28 (C₅₂H₃₄N₄ = 714.85) |
| 3-49-2 | m/z = 714.28 (C₅₂H₃₄N₄ = 714.85) | 3-50-2 | m/z = 715.27 (C₅₁H₃₃N₅ = 715.84) |
| 3-51-2 | m/z = 716.27 (C₅₀H₃₂N₆ = 716.83) | 3-52-2 | m/z = 613.23 (C₄₃H₂₇N₅ = 613.71) |
| 3-53-2 | m/z = 485.19 (C₃₅H₂₃N₃ = 485.58) | 3-54-2 | m/z = 535.20 (C₃₉H₂₅N₃ = 535.64) |
| 3-55-2 | m/z = 561.22 (C₄₁H₂₇N₃ = 561.67) | 3-56-2 | m/z = 640.24 (C₄₄H₂₈N₆ = 640.73) |
| 3-57-2 | m/z = 485.19 (C₃₅H₂₃N₃ = 485.58) | 3-58-2 | m/z = 535.20 (C₃₉H₂₅N₃ = 535.64) |
| 3-59-2 | m/z = 561.22 (C₄₁H₂₇N₃ = 561.67) | 3-60-2 | m/z = 640.24 (C₄₄H₂₈N₆ = 640.73) |
| 3-61-2 | m/z = 485.19 (C₃₅H₂₃N₃ = 485.58) | 3-62-2 | m/z = 561.22 (C₄₁H₂₇N₃ = 561.67) |
| 3-63-2 | m/z = 561.22 (C₄₁H₂₇N₃ = 561.67) | 3-64-2 | m/z = 637.25 (C₄₇H₃₁N₃ = 637.77) |
| 3-65-2 | m/z = 637.25 (C₄₇H₃₁N₃ = 637.77) | 3-66-2 | m/z = 637.25 (C₄₇H₃₁N₃ = 637.77) |
| 3-67-2 | m/z = 637.25 (C₄₇H₃₁N₃ = 637.77) | 3-68-2 | m/z = 640.24 (C₄₄H₂₈N₆ = 640.73) |
| 3-69-2 | m/z = 639.24 (C₄₅H₂₉N₅ = 639.75) | 3-70-2 | m/z = 639.24 (C₄₅H₂₉N₅ = 639.75) |
| 3-71-2 | m/z = 639.24 (C₄₅H₂₉N₅ = 639.75) | 3-72-2 | m/z = 638.25 (C₄₆H₃₀N₄ = 638.76) |
| 3-73-2 | m/z = 638.25 (C₄₆H₃₀N₄ = 638.76) | 3-74-2 | m/z = 639.24 (C₄₅H₂₉N₅ = 639.75) |
| 3-75-2 | m/z = 640.24 (C₄₄H₂₈N₆ = 640.73) | 3-76-2 | m/z = 716.27 (C₅₀H₃₂N₆ = 716.83) |
| 3-77-2 | m/z = 715.27 (C₅₁H₃₃N₅ = 715.84) | 3-78-2 | m/z = 715.27 (C₅₁H₃₃N₅ = 715.84) |
| 3-79-2 | m/z = 714.28 (C₅₂H₃₄N₄ = 714.85) | 3-80-2 | m/z = 714.28 (C₅₂H₃₄N₄ = 714.85) |
| 3-81-2 | m/z = 715.27 (C₅₁H₃₃N₅ = 715.84) | 3-82-2 | m/z = 716.27 (C₅₀H₃₂N₆ = 716.83) |
| 3-83-2 | m/z = 716.27 (C₅₀H₃₂N₆ = 716.83) | 3-84-2 | m/z = 715.27 (C₅₁H₃₃N₅ = 715.84) |
| 3-85-2 | m/z = 715.27 (C₅₁H₃₃N₅ = 715.84) | 3-86-2 | m/z = 714.28 (C₅₂H₃₄N₄ = 714.85) |
| 3-87-2 | m/z = 714.28 (C₅₂H₃₄N₄ = 714.85) | 3-88-2 | m/z = 715.27 (C₅₁H₃₃N₅ = 715.84) |
| 3-89-2 | m/z = 716.27 (C₅₀H₃₂N₆ = 716.83) | 3-90-2 | m/z = 613.23 (C₄₃H₂₇N₅ = 613.71) |
| 3-91-2 | m/z = 640.24 (C₄₄H₂₈N₆ = 640.73) | 3-92-2 | m/z = 639.24 (C₄₅H₂₉N₅ = 639.75) |
| 3-93-2 | m/z = 639.24 (C₄₅H₂₉N₅ = 639.75) | 3-94-2 | m/z = 639.24 (C₄₅H₂₉N₅ = 639.75) |
| 3-95-2 | m/z = 638.25 (C₄₆H₃₀N₄ = 638.76) | 3-96-2 | m/z = 638.25 (C₄₆H₃₀N₄ = 638.76) |
| 3-97-2 | m/z = 639.24 (C₄₅H₂₉N₅ = 639.75) | 3-98-2 | m/z = 640.24 (C₄₄H₂₈N₆ = 640.73) |
| 3-99-2 | m/z = 716.27 (C₅₀H₃₂N₆ = 716.83) | 3-100-2 | m/z = 715.27 (C₅₁H₃₃N₅ = 715.84) |
| 3-101-2 | m/z = 715.27 (C₅₁H₃₃N₅ = 715.84) | 3-102-2 | m/z = 714.28 (C₅₂H₃₄N₄ = 714.85) |
| 3-103-2 | m/z = 714.28 (C₅₂H₃₄N₄ = 714.85) | 3-104-2 | m/z = 715.27 (C₅₁H₃₃N₅ = 715.84) |
| 3-105-2 | m/z = 716.27 (C₅₀H₃₂N₆ = 716.83) | 3-106-2 | m/z = 716.27 (C₅₀H₃₂N₆ = 716.83) |
| 3-107-2 | m/z = 715.27 (C₅₁H₃₃N₅ = 715.84) | 3-108-2 | m/z = 715.27 (C₅₁H₃₃N₅ = 715.84) |
| 3-109-2 | m/z = 714.28 (C₅₂H₃₄N₄ = 714.85) | 3-110-2 | m/z = 714.28 (C₅₂H₃₄N₄ = 714.85) |
| 3-111-2 | m/z = 715.27 (C₅₁H₃₃N₅ = 715.84) | 3-112-2 | m/z = 716.27 (C₅₀H₃₂N₆ = 716.83) |
| 3-113-2 | m/z = 613.23 (C₄₃H₂₇N₅ = 613.71) | 3-114-2 | m/z = 485.19 (C₃₅H₂₃N₃ = 485.58) |
| 3-115-2 | m/z = 535.20 (C₃₉H₂₅N₃ = 535.64) | 3-116-2 | m/z = 561.22 (C₄₁H₂₇N₃ = 561.67) |
| 3-117-2 | m/z = 640.24 (C₄₄H₂₈N₆ = 640.73) | 3-118-2 | m/z = 485.19 (C₃₅H₂₃N₃ = 485.58) |
| 3-119-2 | m/z = 535.20 (C₃₉H₂₅N₃ = 535.64) | 3-120-2 | m/z = 561.22 (C₄₁H₂₇N₃ = 561.67) |
| 3-121-2 | m/z = 640.24 (C₄₄H₂₈N₆ = 640.73) | 3-122-2 | m/z = 485.19 (C₃₅H₂₃N₃ = 485.58) |
| 3-123-2 | m/z = 535.20 (C₃₉H₂₅N₃ = 535.64) | 3-124-2 | m/z = 561.22 (C₄₁H₂₇N₃ = 561.67) |
| 3-125-2 | m/z = 640.24 (C₄₄H₂₈N₆ = 640.73) | 3-126-2 | m/z = 640.24 (C₄₄H₂₈N₆ = 640.73) |
| 3-127-2 | m/z = 535.20 (C₃₉H₂₅N₃ = 535.64) | 3-128-2 | m/z = 535.20 (C₃₉H₂₅N₃ = 535.64) |
| 4-1-2 | m/z = 485.19 (C₃₅H₂₃N₃ = 485.58) | 4-2-2 | m/z = 535.20 (C₃₉H₂₅N₃ = 535.64) |
| 4-3-2 | m/z = 561.22 (C₄₁H₂₇N₃ = 561.67) | 4-4-2 | m/z = 640.24 (C₄₄H₂₈N₆ = 640.73) |
| 4-5-2 | m/z = 485.19 (C₃₅H₂₃N₃ = 485.58) | 4-6-2 | m/z = 535.20 (C₃₉H₂₅N₃ = 535.64) |
| 4-7-2 | m/z = 561.22 (C₄₁H₂₇N₃ = 561.67) | 4-8-2 | m/z = 640.24 (C₄₄H₂₈N₆ = 640.73) |
| 4-9-2 | m/z = 485.19 (C₃₅H₂₃N₃ = 485.58) | 4-10-2 | m/z = 535.20 (C₃₉H₂₅N₃ = 535.64) |
| 4-11-2 | m/z = 561.22 (C₄₁H₂₇N₃ = 561.67) | 4-12-2 | m/z = 640.24 (C₄₄H₂₈N₆ = 640.73) |
| 4-13-2 | m/z = 485.19 (C₃₅H₂₃N₃ = 485.58) | 4-14-2 | m/z = 535.20 (C₃₉H₂₅N₃ = 535.64) |
| 4-15-2 | m/z = 561.22 (C₄₁H₂₇N₃ = 561.67) | 4-16-2 | m/z = 640.24 (C₄₄H₂₈N₆ = 640.73) |
| 4-17-2 | m/z = 485.19 (C₃₅H₂₃N₃ = 485.58) | 4-18-2 | m/z = 535.20 (C₃₉H₂₅N₃ = 535.64) |
| 4-19-2 | m/z = 561.22 (C₄₁H₂₇N₃ = 561.67) | 4-20-2 | m/z = 640.24 (C₄₄H₂₈N₆ = 640.73) |
| 4-21-2 | m/z = 485.19 (C₃₅H₂₃N₃ = 485.58) | 4-22-2 | m/z = 535.20 (C₃₉H₂₅N₃ = 535.64) |
| 4-23-2 | m/z = 561.22 (C₄₁H₂₇N₃ = 561.67) | 4-24-2 | m/z = 640.24 (C₄₄H₂₈N₆ = 640.73) |
| 4-25-2 | m/z = 485.19 (C₃₅H₂₃N₃ = 485.58) | 4-26-2 | m/z = 535.20 (C₃₉H₂₅N₃ = 535.64) |
| 4-27-2 | m/z = 561.22 (C₄₁H₂₇N₃ = 561.67) | 4-28-2 | m/z = 640.24 (C₄₄H₂₈N₆ = 640.73) |
| 5-1-2 | m/z = 653.26 (C₄₆H₃₁N₅ = 653.77) | 5-3-2 | m/z = 652.26 (C₄₇H₃₂N₄ = 652.78) |
| 5-2-2 | m/z = 728.29 (C₅₃H₃₆N₄ = 728.88) | 5-4-2 | m/z = 728.29 (C₅₃H₃₆N₄ = 728.88) |

### Manufacture and Evaluation of Organic Electronic Element

### □. Manufacture and Test of Green Organic Light Emitting Element (Phosphorescent Host)

### Example 2-1 Green Organic Light Emitting Element (Phosphorescent Host)

An organic electronic light emitting element was manufactured by an ordinary method using the compound obtained through synthesis as a host material for a light emitting layer. First, N¹-(naphthalen-2-yl)-N⁴,N⁴-bis(4-(naphthalen-2-yl(phenyl)amino)phenyl)-N¹-phenylbenzene-1,4-diamine (hereinafter, abbreviated as “2-TNATA”) was vacuum-deposited on an ITO layer (anode) formed on a galas substrate, to form a hole injection layer with a thickness 60 nm. Then, 4,4-bis[N-(1-naphthyl)-N-phenylamino]biphenyl (hereinafter, abbreviated as “-NPD”) as a hole transport compound was vacuum-deposited on the hole injection layer, to form a hole transport layer with a thickness of 60 nm. Subsequently, a light emitting layer with a thickness of 30 nm was formed on the hole transport layer by doping an upper portion of the hole transport layer with the compound 1-1-2 of the present invention as a host and Ir(ppy)₃ [tris(2-phenylpyridine)-iridium] as a dopant at a weight ratio of 95:5. Then, (1.1′-bisphenyl)-4-olato)bis(2-methyl-8-quinolinolato)aluminum (hereinafter, abbreviated as “BAlq”) was vacuum-deposited with a thickness of 10 nm for a hole blocking layer, and tris(8-quinolinol)aluminum (hereinafter, abbreviated as “Alq3”) was formed with a thickness of 40 nm for an electron injection layer. Thereafter, LiF as halogenated alkali metal was deposited with a thickness of 0.2 nm, and subsequently Al was deposited with a thickness of 150 nm, thereby using this Al/LiF as a cathode. In this way, an organic electronic light emitting element was manufactured.

### Example 2-2 to Example 2-312 Green Organic Light Emitting Element (Phosphorescent Host)

An organic electronic light emitting element was manufactured by the same method as in Example 2-1 except that, instead of compound 1-1-2 of the present invention, one of compounds 1-2-2 to 1-28-1, 2-1-2 to 2-128-2, 3-1-2 to 3-128-2, and 4-1-2 to 4-28-2 of the present invention listed on table 5 below was used as a phosphorescent host material for a light emitting layer.

### Comparative Example 2-1

An organic electronic light emitting element was manufactured by the same method as in Example 2-1 except that, instead of compound 2-1-1 of the present invention, comparative compound A [4,4′-N,N′-dicarbazole-biphenyl (CBP)] described in <Example 1> was used as a phosphorescent host material for a light emitting layer.

### Comparative Example 2-2

An organic electronic light emitting element was manufactured by the same method as in Example 2-1 except that, instead of compound 1-1-2 of the present invention, comparative compound B described in <Example 1> was used as a phosphorescent host material for a light emitting layer.

### Comparative Example 2-3

An organic electronic light emitting element was manufactured by the same method as in Example 1-1 except that, instead of compound 1-1-2 of the present invention, comparative compound C described in <Example 1> was used as a phosphorescent host material for a light emitting layer.

### Comparative Example 2-4

An organic electronic light emitting element was manufactured by the same method as in Example 1-1 except that, instead of compound 1-1-2 of the present invention, comparative compound D described in <Example 1> was used as a phosphorescent host material for a light emitting layer.

A forward bias DC voltage was applied to the organic electronic light emitting elements manufactured in Examples 2-1 to 2-312 and Comparative Examples 2-1 to 2-4 to measure electro-luminescence (EL) characteristics thereof by PR-650 (Photoresearch), and the T95 lifetime was measured by lifetime measuring equipments (Mcscience) at reference brightness of 5000 cd/m². Table 2-5 below shows the manufacture of elements and evaluation results thereof.

TABLE 2-5

| | | | Current | Brightness | | Lifetime | CIE |
|---|---|---|---|---|---|---|---|
| | Compound | Voltage | Density | (cd/m2) | Efficiency | T (95) | (x, y) |
| Comparative | Compound | 5.8 | 23.1 | 5000.0 | 21.6 | 65.8 | (0.31, 0.60) |
| Example | (A) | | | | | | |
| (2-1) | | | | | | | |
| Comparative | Compound | 5.2 | 16.9 | 5000.0 | 29.5 | 88.7 | (0.31, 0.61) |
| Example | (B) | | | | | | |
| (2-2) | | | | | | | |
| Comparative | Compound | 5.4 | 18.7 | 5000.0 | 26.7 | 81.1 | (0.31, 0.60) |
| Example | (C) | | | | | | |
| (2-3) | | | | | | | |
| Comparative | Compound | 5.5 | 17.3 | 5000.0 | 28.9 | 84.3 | (0.33, 0.61) |
| Example | (D) | | | | | | |
| (2-4) | | | | | | | |
| Example | Compound | 4.8 | 13.3 | 5000.0 | 37.6 | 140.5 | (0.30, 0.60) |
| (2-1) | (1-1-2) | | | | | | |
| Example | Compound | 4.7 | 14.8 | 5000.0 | 33.7 | 109.9 | (0.31, 0.61) |
| (2-2) | (1-2-2) | | | | | | |
| Example | Compound | 4.8 | 13.5 | 5000.0 | 36.9 | 92.4 | (0.31, 0.60) |
| (2-3) | (1-3-2) | | | | | | |
| Example | Compound | 4.5 | 15.3 | 5000.0 | 32.6 | 140.3 | (0.33, 0.61) |
| (2-4) | (1-4-2) | | | | | | |
| Example | Compound | 4.8 | 12.6 | 5000.0 | 39.6 | 92.4 | (0.32, 0.61) |
| (2-5) | (1-5-2) | | | | | | |
| Example | Compound | 4.5 | 13.5 | 5000.0 | 37.1 | 106.1 | (0.33, 0.60) |
| (2-6) | (1-6-2) | | | | | | |
| Example | Compound | 4.9 | 13.6 | 5000.0 | 36.8 | 102.1 | (0.32, 0.61) |
| (2-7) | (1-7-2) | | | | | | |
| Example | Compound | 4.9 | 15.1 | 5000.0 | 33.1 | 123.4 | (0.31, 0.60) |
| (2-8) | (1-8-2) | | | | | | |
| Example | Compound | 4.6 | 13.4 | 5000.0 | 37.4 | 91.7 | (0.31, 0.61) |
| (2-9) | (1-9-2) | | | | | | |
| Example | Compound | 4.9 | 16.2 | 5000.0 | 30.8 | 98.3 | (0.31, 0.60) |
| (2-10) | (1-10-2) | | | | | | |
| Example | Compound | 4.6 | 12.7 | 5000.0 | 39.2 | 123.5 | (0.33, 0.61) |
| (2-11) | (1-11-2) | | | | | | |
| Example | Compound | 4.8 | 14.8 | 5000.0 | 33.8 | 134.1 | (0.30, 0.60) |
| (2-12) | (1-12-2) | | | | | | |
| Example | Compound | 4.8 | 15.7 | 5000.0 | 31.9 | 128.0 | (0.31, 0.61) |
| (2-13) | (1-13-2) | | | | | | |
| Example | Compound | 5.0 | 13.8 | 5000.0 | 36.2 | 128.0 | (0.31, 0.60) |
| (2-14) | (1-14-2) | | | | | | |
| Example | Compound | 4.9 | 12.8 | 5000.0 | 39.2 | 131.0 | (0.33, 0.61) |
| (2-15) | (1-15-2) | | | | | | |
| Example | Compound | 4.9 | 14.8 | 5000.0 | 33.8 | 125.8 | (0.32, 0.61) |
| (2-16) | (1-16-2) | | | | | | |
| Example | Compound | 4.6 | 12.8 | 5000.0 | 38.9 | 99.7 | (0.33, 0.60) |
| (2-17) | (1-17-2) | | | | | | |
| Example | Compound | 5.0 | 15.2 | 5000.0 | 32.9 | 149.9 | (0.32, 0.61) |
| (2-18) | (1-18-2) | | | | | | |
| Example | Compound | 4.9 | 12.7 | 5000.0 | 39.4 | 90.1 | (0.31, 0.60) |
| (2-19) | (1-19-2) | | | | | | |
| Example | Compound | 4.7 | 16.3 | 5000.0 | 30.6 | 108.1 | (0.31, 0.61) |
| (2-20) | (1-20-2) | | | | | | |
| Example | Compound | 4.7 | 14.8 | 5000.0 | 33.7 | 98.4 | (0.31, 0.60) |
| (2-21) | (1-21-2) | | | | | | |
| Example | Compound | 4.5 | 16.5 | 5000.0 | 30.3 | 114.2 | (0.33, 0.61) |
| (2-22) | (1-22-2) | | | | | | |
| Example | Compound | 5.0 | 16.5 | 5000.0 | 30.3 | 131.2 | (0.30, 0.60) |
| (2-23) | (1-23-2) | | | | | | |
| Example | Compound | 4.8 | 14.9 | 5000.0 | 33.5 | 138.0 | (0.31, 0.61) |
| (2-24) | (1-24-2) | | | | | | |
| Example | Compound | 4.6 | 12.9 | 5000.0 | 38.9 | 145.7 | (0.31, 0.60) |
| (2-25) | (1-25-2) | | | | | | |
| Example | Compound | 4.5 | 14.9 | 5000.0 | 33.5 | 95.4 | (0.33, 0.61) |
| (2-26) | (1-26-2) | | | | | | |
| Example | Compound | 4.7 | 12.7 | 5000.0 | 39.5 | 139.5 | (0.32, 0.61) |
| (2-27) | (1-27-2) | | | | | | |
| Example | Compound | 4.9 | 14.3 | 5000.0 | 34.9 | 125.5 | (0.33, 0.60) |
| (2-28) | (1-28-2) | | | | | | |
| Example | Compound | 4.8 | 14.9 | 5000.0 | 33.5 | 100.3 | (0.31, 0.61) |
| (2-29) | (2-1-2) | | | | | | |
| Example | Compound | 4.6 | 13.7 | 5000.0 | 36.5 | 98.6 | (0.31, 0.60) |
| (2-30) | (2-2-2) | | | | | | |
| Example | Compound | 4.6 | 13.4 | 5000.0 | 37.3 | 95.8 | (0.33, 0.61) |
| (2-31) | (2-3-2) | | | | | | |
| Example | Compound | 4.8 | 16.0 | 5000.0 | 31.2 | 107.1 | (0.32, 0.61) |
| (2-32) | (2-4-2) | | | | | | |
| Example | Compound | 4.7 | 12.9 | 5000.0 | 38.8 | 131.4 | (0.33, 0.60) |
| (2-33) | (2-5-2) | | | | | | |
| Example | Compound | 4.8 | 15.3 | 5000.0 | 32.6 | 139.2 | (0.32, 0.61) |
| (2-34) | (2-6-2) | | | | | | |
| Example | Compound | 4.5 | 16.4 | 5000.0 | 30.4 | 109.9 | (0.31, 0.60) |
| (2-35) | (2-7-2) | | | | | | |
| Example | Compound | 5.0 | 16.0 | 5000.0 | 31.2 | 147.3 | (0.31, 0.61) |
| (2-36) | (2-8-2) | | | | | | |
| Example | Compound | 5.0 | 12.6 | 5000.0 | 39.6 | 134.4 | (0.31, 0.60) |
| (2-37) | (2-9-2) | | | | | | |
| Example | Compound | 4.9 | 12.8 | 5000.0 | 39.2 | 95.4 | (0.33, 0.61) |
| (2-38) | (2-10-2) | | | | | | |
| Example | Compound | 5.0 | 16.6 | 5000.0 | 30.1 | 147.1 | (0.30, 0.60) |
| (2-39) | (2-11-2) | | | | | | |
| Example | Compound | 4.9 | 13.4 | 5000.0 | 37.3 | 111.2 | (0.31, 0.61) |
| (2-40) | (2-12-2) | | | | | | |
| Example | Compound | 4.6 | 16.1 | 5000.0 | 31.0 | 111.7 | (0.31, 0.60) |
| (2-41) | (2-13-2) | | | | | | |
| Example | Compound | 4.9 | 14.8 | 5000.0 | 33.8 | 133.2 | (0.33, 0.61) |
| (2-42) | (2-14-2) | | | | | | |
| Example | Compound | 4.6 | 13.3 | 5000.0 | 37.5 | 111.3 | (0.32, 0.61) |
| (2-43) | (2-15-2) | | | | | | |
| Example | Compound | 4.5 | 16.5 | 5000.0 | 30.3 | 120.5 | (0.33, 0.60) |
| (2-44) | (2-16-2) | | | | | | |
| Example | Compound | 4.7 | 12.7 | 5000.0 | 39.5 | 126.0 | (0.32, 0.61) |
| (2-45) | (2-17-2) | | | | | | |
| Example | Compound | 4.6 | 13.1 | 5000.0 | 38.2 | 136.3 | (0.31, 0.60) |
| (2-46) | (2-18-2) | | | | | | |
| Example | Compound | 4.5 | 16.5 | 5000.0 | 30.3 | 134.4 | (0.31, 0.61) |
| (2-47) | (2-19-2) | | | | | | |
| Example | Compound | 4.6 | 16.3 | 5000.0 | 30.6 | 141.5 | (0.31, 0.60) |
| (2-48) | (2-20-2) | | | | | | |
| Example | Compound | 4.8 | 14.4 | 5000.0 | 34.8 | 146.4 | (0.33, 0.61) |
| (2-49) | (2-21-2) | | | | | | |
| Example | Compound | 4.7 | 14.4 | 5000.0 | 34.8 | 123.3 | (0.30, 0.60) |
| (2-50) | (2-22-2) | | | | | | |
| Example | Compound | 4.8 | 15.2 | 5000.0 | 32.9 | 123.3 | (0.31, 0.61) |
| (2-51) | (2-23-2) | | | | | | |
| Example | Compound | 4.7 | 15.1 | 5000.0 | 33.2 | 116.8 | (0.31, 0.60) |
| (2-52) | (2-24-2) | | | | | | |
| Example | Compound | 4.9 | 15.6 | 5000.0 | 32.1 | 131.1 | (0.33, 0.61) |
| (2-53) | (2-25-2) | | | | | | |
| Example | Compound | 4.9 | 13.9 | 5000.0 | 36.0 | 145.8 | (0.32, 0.61) |
| (2-54) | (2-26-2) | | | | | | |
| Example | Compound | 4.7 | 14.1 | 5000.0 | 35.6 | 130.0 | (0.33, 0.60) |
| (2-55) | (2-27-2) | | | | | | |
| Example | Compound | 4.6 | 15.2 | 5000.0 | 32.8 | 127.8 | (0.32, 0.61) |
| (2-56) | (2-28-2) | | | | | | |
| Example | Compound | 4.6 | 13.3 | 5000.0 | 37.7 | 116.0 | (0.31, 0.60) |
| (2-57) | (2-29-2) | | | | | | |
| Example | Compound | 4.6 | 12.9 | 5000.0 | 38.7 | 116.7 | (0.33, 0.61) |
| (2-58) | (2-30-2) | | | | | | |
| Example | Compound | 5.0 | 15.3 | 5000.0 | 32.8 | 133.9 | (0.30, 0.60) |
| (2-59) | (2-31-2) | | | | | | |
| Example | Compound | 4.6 | 15.3 | 5000.0 | 32.7 | 137.4 | (0.31, 0.61) |
| (2-60) | (2-32-2) | | | | | | |
| Example | Compound | 4.8 | 16.3 | 5000.0 | 30.6 | 149.3 | (0.31, 0.60) |
| (2-61) | (2-33-2) | | | | | | |
| Example | Compound | 4.7 | 14.0 | 5000.0 | 35.6 | 125.4 | (0.33, 0.61) |
| (2-62) | (2-34-2) | | | | | | |
| Example | Compound | 4.5 | 14.7 | 5000.0 | 34.1 | 132.4 | (0.32, 0.61) |
| (2-63) | (2-35-2) | | | | | | |
| Example | Compound | 4.8 | 14.5 | 5000.0 | 34.5 | 148.2 | (0.33, 0.60) |
| (2-64) | (2-36-2) | | | | | | |
| Example | Compound | 4.9 | 15.3 | 5000.0 | 32.7 | 118.4 | (0.32, 0.61) |
| (2-65) | (2-37-2) | | | | | | |
| Example | Compound | 4.6 | 14.3 | 5000.0 | 35.1 | 139.2 | (0.31, 0.60) |
| (2-66) | (2-38-2) | | | | | | |
| Example | Compound | 5.0 | 13.8 | 5000.0 | 36.3 | 105.6 | (0.31, 0.61) |
| (2-67) | (2-39-2) | | | | | | |
| Example | Compound | 4.7 | 14.4 | 5000.0 | 34.8 | 122.7 | (0.31, 0.60) |
| (2-68) | (2-40-2) | | | | | | |
| Example | Compound | 4.6 | 14.1 | 5000.0 | 35.5 | 142.5 | (0.33, 0.61) |
| (2-69) | (2-41-2) | | | | | | |
| Example | Compound | 4.9 | 13.1 | 5000.0 | 38.1 | 120.4 | (0.30, 0.60) |
| (2-70) | (2-42-2) | | | | | | |
| Example | Compound | 4.8 | 13.8 | 5000.0 | 36.3 | 123.8 | (0.31, 0.61) |
| (2-71) | (2-43-2) | | | | | | |
| Example | Compound | 4.6 | 14.0 | 5000.0 | 35.7 | 94.7 | (0.31, 0.60) |
| (2-72) | (2-44-2) | | | | | | |
| Example | Compound | 4.7 | 14.2 | 5000.0 | 35.1 | 107.2 | (0.33, 0.61) |
| (2-73) | (2-45-2) | | | | | | |
| Example | Compound | 5.0 | 16.2 | 5000.0 | 30.9 | 134.2 | (0.32, 0.61) |
| (2-74) | (2-46-2) | | | | | | |
| Example | Compound | 4.6 | 13.4 | 5000.0 | 37.4 | 99.8 | (0.33, 0.61) |
| (2-75) | (2-47-2) | | | | | | |
| Example | Compound | 4.7 | 14.6 | 5000.0 | 34.1 | 105.8 | (0.30, 0.60) |
| (2-76) | (2-48-2) | | | | | | |
| Example | Compound | 4.7 | 15.7 | 5000.0 | 31.8 | 114.4 | (0.31, 0.61) |
| (2-77) | (2-49-2) | | | | | | |
| Example | Compound | 4.5 | 15.4 | 5000.0 | 32.4 | 105.8 | (0.31, 0.60) |
| (2-78) | (2-50-2) | | | | | | |
| Example | Compound | 4.5 | 13.8 | 5000.0 | 36.3 | 132.0 | (0.31, 0.61) |
| (2-79) | (2-51-2) | | | | | | |
| Example | Compound | 4.6 | 13.2 | 5000.0 | 37.9 | 144.8 | (0.31, 0.60) |
| (2-80) | (2-52-2) | | | | | | |
| Example | Compound | 4.6 | 15.8 | 5000.0 | 31.7 | 135.7 | (0.33, 0.61) |
| (2-81) | (2-53-2) | | | | | | |
| Example | Compound | 4.6 | 12.7 | 5000.0 | 39.5 | 123.8 | (0.32, 0.61) |
| (2-82) | (2-54-2) | | | | | | |
| Example | Compound | 4.6 | 13.4 | 5000.0 | 37.4 | 109.7 | (0.33, 0.60) |
| (2-83) | (2-55-2) | | | | | | |
| Example | Compound | 4.8 | 14.0 | 5000.0 | 35.6 | 129.2 | (0.32, 0.61) |
| (2-84) | (2-56-2) | | | | | | |
| Example | Compound | 4.9 | 15.5 | 5000.0 | 32.2 | 146.8 | (0.31, 0.60) |
| (2-85) | (2-57-2) | | | | | | |
| Example | Compound | 4.7 | 13.2 | 5000.0 | 37.9 | 149.4 | (0.31, 0.61) |
| (2-86) | (2-58-2) | | | | | | |
| Example | Compound | 4.8 | 15.8 | 5000.0 | 31.6 | 147.9 | (0.31, 0.60) |
| (2-87) | (2-59-2) | | | | | | |
| Example | Compound | 4.9 | 12.8 | 5000.0 | 39.1 | 132.1 | (0.33, 0.61) |
| (2-88) | (2-60-2) | | | | | | |
| Example | Compound | 4.7 | 13.9 | 5000.0 | 36.0 | 112.7 | (0.30, 0.60) |
| (2-89) | (2-61-2) | | | | | | |
| Example | Compound | 4.9 | 14.3 | 5000.0 | 34.9 | 138.1 | (0.31, 0.61) |
| (2-90) | (2-62-2) | | | | | | |
| Example | Compound | 4.7 | 13.4 | 5000.0 | 37.4 | 142.7 | (0.31, 0.60) |
| (2-91) | (2-63-2) | | | | | | |
| Example | Compound | 5.0 | 16.5 | 5000.0 | 30.3 | 145.3 | (0.33, 0.61) |
| (2-92) | (2-64-2) | | | | | | |
| Example | Compound | 4.9 | 14.7 | 5000.0 | 34.0 | 101.2 | (0.32, 0.61) |
| (2-93) | (2-65-2) | | | | | | |
| Example | Compound | 4.8 | 14.6 | 5000.0 | 34.3 | 120.8 | (0.33, 0.60) |
| (2-94) | (2-66-2) | | | | | | |
| Example | Compound | 4.7 | 15.1 | 5000.0 | 33.2 | 140.2 | (0.32, 0.61) |
| (2-95) | (2-67-2) | | | | | | |
| Example | Compound | 4.9 | 13.0 | 5000.0 | 38.4 | 106.9 | (0.31, 0.60) |
| (2-96) | (2-68-2) | | | | | | |
| Example | Compound | 4.6 | 13.7 | 5000.0 | 36.6 | 132.7 | (0.31, 0.61) |
| (2-97) | (2-69-2) | | | | | | |
| Example | Compound | 4.7 | 16.0 | 5000.0 | 31.2 | 122.8 | (0.31, 0.60) |
| (2-98) | (2-70-2) | | | | | | |
| Example | Compound | 4.6 | 15.4 | 5000.0 | 32.5 | 122.8 | (0.33, 0.61) |
| (2-99) | (2-71-2) | | | | | | |
| Example | Compound | 4.9 | 15.9 | 5000.0 | 31.5 | 118.3 | (0.30, 0.60) |
| (2-100) | (2-72-2) | | | | | | |
| Example | Compound | 4.7 | 13.4 | 5000.0 | 37.3 | 99.8 | (0.31, 0.61) |
| (2-101) | (2-73-2) | | | | | | |
| Example | Compound | 4.9 | 13.4 | 5000.0 | 37.4 | 90.4 | (0.31, 0.60) |
| (2-102) | (2-74-2) | | | | | | |
| Example | Compound | 4.8 | 12.8 | 5000.0 | 39.0 | 143.2 | (0.33, 0.61) |
| (2-103) | (2-75-2) | | | | | | |
| Example | Compound | 4.8 | 16.5 | 5000.0 | 30.4 | 127.7 | (0.32, 0.61) |
| (2-104) | (2-76-2) | | | | | | |
| Example | Compound | 4.6 | 13.4 | 5000.0 | 37.4 | 123.1 | (0.33, 0.60) |
| (2-105) | (2-77-2) | | | | | | |
| Example | Compound | 4.9 | 13.1 | 5000.0 | 38.2 | 110.8 | (0.32, 0.61) |
| (2-106) | (2-78-2) | | | | | | |
| Example | Compound | 4.6 | 16.2 | 5000.0 | 30.9 | 94.1 | (0.31, 0.60) |
| (2-107) | (2-79-2) | | | | | | |
| Example | Compound | 4.5 | 14.2 | 5000.0 | 35.1 | 124.0 | (0.33, 0.61) |
| (2-108) | (2-80-2) | | | | | | |
| Example | Compound | 4.9 | 16.1 | 5000.0 | 31.1 | 141.3 | (0.30, 0.60) |
| (2-109) | (2-81-2) | | | | | | |
| Example | Compound | 4.8 | 13.0 | 5000.0 | 38.6 | 134.6 | (0.31, 0.61) |
| (2-110) | (2-82-2) | | | | | | |
| Example | Compound | 4.5 | 12.7 | 5000.0 | 39.5 | 147.8 | (0.31, 0.60) |
| (2-111) | (2-83-2) | | | | | | |
| Example | Compound | 4.7 | 16.4 | 5000.0 | 30.5 | 100.4 | (0.33, 0.61) |
| (2-112) | (2-84-2) | | | | | | |
| Example | Compound | 5.0 | 13.6 | 5000.0 | 36.7 | 96.0 | (0.32, 0.61) |
| (2-113) | (2-85-2) | | | | | | |
| Example | Compound | 4.8 | 14.5 | 5000.0 | 34.5 | 132.0 | (0.33, 0.60) |
| (2-114) | (2-86-2) | | | | | | |
| Example | Compound | 4.6 | 13.0 | 5000.0 | 38.6 | 94.8 | (0.32, 0.61) |
| (2-115) | (2-87-2) | | | | | | |
| Example | Compound | 4.7 | 12.5 | 5000.0 | 39.9 | 146.9 | (0.31, 0.60) |
| (2-116) | (2-88-2) | | | | | | |
| Example | Compound | 4.7 | 12.8 | 5000.0 | 39.0 | 127.2 | (0.31, 0.61) |
| (2-117) | (2-89-2) | | | | | | |
| Example | Compound | 5.0 | 16.0 | 5000.0 | 31.3 | 97.9 | (0.31, 0.60) |
| (2-118) | (2-90-2) | | | | | | |
| Example | Compound | 4.9 | 16.1 | 5000.0 | 31.1 | 147.8 | (0.33, 0.61) |
| (2-119) | (2-91-2) | | | | | | |
| Example | Compound | 4.7 | 14.9 | 5000.0 | 33.5 | 119.0 | (0.30, 0.60) |
| (2-120) | (2-92-2) | | | | | | |
| Example | Compound | 4.9 | 16.0 | 5000.0 | 31.2 | 140.0 | (0.31, 0.61) |
| (2-121) | (2-93-2) | | | | | | |
| Example | Compound | 4.9 | 14.4 | 5000.0 | 34.7 | 108.1 | (0.31, 0.60) |
| (2-122) | (2-94-2) | | | | | | |
| Example | Compound | 4.8 | 12.6 | 5000.0 | 39.5 | 124.2 | (0.33, 0.61) |
| (2-123) | (2-95-2) | | | | | | |
| Example | Compound | 4.6 | 15.7 | 5000.0 | 31.8 | 141.9 | (0.32, 0.61) |
| (2-124) | (2-96-2) | | | | | | |
| Example | Compound | 4.9 | 14.5 | 5000.0 | 34.4 | 90.0 | (0.33, 0.61) |
| (2-125) | (2-97-2) | | | | | | |
| Example | Compound | 4.9 | 15.8 | 5000.0 | 31.7 | 128.4 | (0.30, 0.60) |
| (2-126) | (2-98-2) | | | | | | |
| Example | Compound | 4.8 | 16.2 | 5000.0 | 30.8 | 125.5 | (0.32, 0.61) |
| (2-127) | (2-99-2) | | | | | | |
| Example | Compound | 4.7 | 14.0 | 5000.0 | 35.7 | 107.6 | (0.31, 0.60) |
| (2-128) | (2-100-2) | | | | | | |
| Example | Compound | 4.6 | 13.9 | 5000.0 | 36.1 | 129.5 | (0.30, 0.60) |
| (2-129) | (2-101-2) | | | | | | |
| Example | Compound | 5.0 | 12.6 | 5000.0 | 39.6 | 146.9 | (0.31, 0.61) |
| (2-130) | (2-102-2) | | | | | | |
| Example | Compound | 4.6 | 16.7 | 5000.0 | 30.0 | 92.0 | (0.31, 0.60) |
| (2-131) | (2-103-2) | | | | | | |
| Example | Compound | 4.6 | 15.8 | 5000.0 | 31.7 | 149.8 | (0.33, 0.61) |
| (2-132) | (2-104-2) | | | | | | |
| Example | Compound | 4.7 | 15.9 | 5000.0 | 31.4 | 103.9 | (0.32, 0.61) |
| (2-133) | (2-105-2) | | | | | | |
| Example | Compound | 4.9 | 15.5 | 5000.0 | 32.3 | 121.5 | (0.33, 0.60) |
| (2-134) | (2-106-2) | | | | | | |
| Example | Compound | 4.6 | 12.6 | 5000.0 | 39.8 | 96.3 | (0.32, 0.61) |
| (2-135) | (2-107-2) | | | | | | |
| Example | Compound | 4.6 | 12.8 | 5000.0 | 39.2 | 115.0 | (0.31, 0.60) |
| (2-136) | (2-108-2) | | | | | | |
| Example | Compound | 4.7 | 16.5 | 5000.0 | 30.4 | 147.4 | (0.31, 0.61) |
| (2-137) | (2-109-2) | | | | | | |
| Example | Compound | 4.6 | 13.0 | 5000.0 | 38.4 | 99.5 | (0.31, 0.60) |
| (2-138) | (2-110-2) | | | | | | |
| Example | Compound | 4.5 | 13.2 | 5000.0 | 38.0 | 112.0 | (0.33, 0.61) |
| (2-139) | (2-111-2) | | | | | | |
| Example | Compound | 4.6 | 15.1 | 5000.0 | 33.2 | 92.7 | (0.30, 0.60) |
| (2-140) | (2-112-2) | | | | | | |
| Example | Compound | 4.7 | 15.0 | 5000.0 | 33.3 | 101.8 | (0.31, 0.61) |
| (2-141) | (2-113-2) | | | | | | |
| Example | Compound | 4.9 | 13.3 | 5000.0 | 37.6 | 92.5 | (0.31, 0.60) |
| (2-142) | (2-114-2) | | | | | | |
| Example | Compound | 4.9 | 13.7 | 5000.0 | 36.5 | 95.2 | (0.33, 0.61) |
| (2-143) | (2-115-2) | | | | | | |
| Example | Compound | 4.6 | 14.2 | 5000.0 | 35.3 | 114.2 | (0.32, 0.61) |
| (2-144) | (2-116-2) | | | | | | |
| Example | Compound | 4.5 | 15.5 | 5000.0 | 32.3 | 145.0 | (0.33, 0.60) |
| (2-145) | (2-117-2) | | | | | | |
| Example | Compound | 4.6 | 13.9 | 5000.0 | 35.9 | 145.8 | (0.32, 0.61) |
| (2-146) | (2-118-2) | | | | | | |
| Example | Compound | 4.5 | 14.0 | 5000.0 | 35.6 | 128.4 | (0.31, 0.60) |
| (2-147) | (2-119-2) | | | | | | |
| Example | Compound | 4.6 | 13.8 | 5000.0 | 36.3 | 110.6 | (0.31, 0.61) |
| (2-148) | (2-120-2) | | | | | | |
| Example | Compound | 4.6 | 16.1 | 5000.0 | 31.0 | 109.6 | (0.31, 0.60) |
| (2-149) | (2-121-2) | | | | | | |
| Example | Compound | 4.9 | 13.6 | 5000.0 | 36.7 | 148.4 | (0.33, 0.61) |
| (2-150) | (2-122-2) | | | | | | |
| Example | Compound | 4.8 | 14.4 | 5000.0 | 34.6 | 126.9 | (0.30, 0.60) |
| (2-151) | (2-123-2) | | | | | | |
| Example | Compound | 4.6 | 12.5 | 5000.0 | 39.9 | 111.1 | (0.31, 0.61) |
| (2-152) | (2-124-2) | | | | | | |
| Example | Compound | 4.7 | 13.3 | 5000.0 | 37.7 | 118.7 | (0.31, 0.60) |
| (2-153) | (2-125-2) | | | | | | |
| Example | Compound | 4.6 | 16.4 | 5000.0 | 30.5 | 95.1 | (0.33, 0.61) |
| (2-154) | (2-126-2) | | | | | | |
| Example | Compound | 4.8 | 13.5 | 5000.0 | 37.0 | 102.4 | (0.32, 0.61) |
| (2-155) | (2-127-2) | | | | | | |
| Example | Compound | 4.8 | 15.7 | 5000.0 | 31.8 | 106.3 | (0.33, 0.60) |
| (2-156) | (2-128-2) | | | | | | |
| Example | Compound | 4.4 | 12.5 | 5000.0 | 40.0 | 122.0 | (0.31, 0.61) |
| (2-157) | (3-1-2) | | | | | | |
| Example | Compound | 4.4 | 12.9 | 5000.0 | 38.8 | 144.8 | (0.31, 0.60) |
| (2-158) | (3-2-2) | | | | | | |
| Example | Compound | 4.5 | 14.0 | 5000.0 | 35.8 | 97.4 | (0.33, 0.61) |
| (2-159) | (3-3-2) | | | | | | |
| Example | Compound | 4.4 | 13.7 | 5000.0 | 36.5 | 124.4 | (0.32, 0.61) |
| (2-160) | (3-4-2) | | | | | | |
| Example | Compound | 4.4 | 13.0 | 5000.0 | 38.6 | 102.8 | (0.33, 0.60) |
| (2-161) | (3-5-2) | | | | | | |
| Example | Compound | 4.5 | 13.7 | 5000.0 | 36.6 | 108.0 | (0.32, 0.61) |
| (2-162) | (3-6-2) | | | | | | |
| Example | Compound | 4.4 | 14.0 | 5000.0 | 35.8 | 93.0 | (0.31, 0.60) |
| (2-163) | (3-7-2) | | | | | | |
| Example | Compound | 4.5 | 13.0 | 5000.0 | 38.4 | 134.3 | (0.31, 0.61) |
| (2-164) | (3-8-2) | | | | | | |
| Example | Compound | 4.5 | 13.6 | 5000.0 | 36.6 | 96.8 | (0.31, 0.60) |
| (2-165) | (3-9-2) | | | | | | |
| Example | Compound | 4.4 | 13.9 | 5000.0 | 35.9 | 123.8 | (0.33, 0.61) |
| (2-166) | (3-10-2) | | | | | | |
| Example | Compound | 4.5 | 13.3 | 5000.0 | 37.6 | 120.6 | (0.30, 0.60) |
| (2-167) | (3-11-2) | | | | | | |
| Example | Compound | 4.5 | 13.6 | 5000.0 | 36.9 | 149.0 | (0.31, 0.61) |
| (2-168) | (3-12-2) | | | | | | |
| Example | Compound | 4.5 | 13.9 | 5000.0 | 36.0 | 135.2 | (0.31, 0.60) |
| (2-169) | (3-13-2) | | | | | | |
| Example | Compound | 4.5 | 13.0 | 5000.0 | 38.5 | 109.9 | (0.33, 0.61) |
| (2-170) | (3-14-2) | | | | | | |
| Example | Compound | 4.4 | 14.1 | 5000.0 | 35.3 | 113.8 | (0.32, 0.61) |
| (2-171) | (3-15-2) | | | | | | |
| Example | Compound | 4.4 | 13.0 | 5000.0 | 38.5 | 141.4 | (0.33, 0.60) |
| (2-172) | (3-16-2) | | | | | | |
| Example | Compound | 4.4 | 13.4 | 5000.0 | 37.2 | 129.4 | (0.32, 0.61) |
| (2-173) | (3-17-2) | | | | | | |
| Example | Compound | 4.4 | 13.0 | 5000.0 | 38.5 | 96.0 | (0.31, 0.60) |
| (2-174) | (3-18-2) | | | | | | |
| Example | Compound | 4.4 | 13.4 | 5000.0 | 37.3 | 128.7 | (0.31, 0.61) |
| (2-175) | (3-19-2) | | | | | | |
| Example | Compound | 4.4 | 13.7 | 5000.0 | 36.5 | 108.6 | (0.31, 0.60) |
| (2-176) | (3-20-2) | | | | | | |
| Example | Compound | 4.4 | 13.5 | 5000.0 | 37.0 | 124.0 | (0.33, 0.61) |
| (2-177) | (3-21-2) | | | | | | |
| Example | Compound | 4.4 | 13.1 | 5000.0 | 38.2 | 133.4 | (0.30, 0.60) |
| (2-178) | (3-22-2) | | | | | | |
| Example | Compound | 4.5 | 12.8 | 5000.0 | 38.9 | 108.2 | (0.31, 0.61) |
| (2-179) | (3-23-2) | | | | | | |
| Example | Compound | 4.5 | 13.6 | 5000.0 | 36.7 | 139.0 | (0.31, 0.60) |
| (2-180) | (3-24-2) | | | | | | |
| Example | Compound | 4.4 | 13.9 | 5000.0 | 35.9 | 98.1 | (0.33, 0.61) |
| (2-181) | (3-25-2) | | | | | | |
| Example | Compound | 4.4 | 13.7 | 5000.0 | 36.6 | 129.5 | (0.32, 0.61) |
| (2-182) | (3-26-2) | | | | | | |
| Example | Compound | 4.5 | 14.1 | 5000.0 | 35.5 | 149.9 | (0.33, 0.60) |
| (2-183) | (3-27-2) | | | | | | |
| Example | Compound | 4.5 | 13.5 | 5000.0 | 37.0 | 125.9 | (0.32, 0.61) |
| (2-184) | (3-28-2) | | | | | | |
| Example | Compound | 4.4 | 13.0 | 5000.0 | 38.5 | 138.1 | (0.31, 0.60) |
| (2-185) | (3-29-2) | | | | | | |
| Example | Compound | 4.5 | 12.7 | 5000.0 | 39.3 | 101.4 | (0.33, 0.61) |
| (2-186) | (3-30-2) | | | | | | |
| Example | Compound | 4.5 | 13.3 | 5000.0 | 37.7 | 149.3 | (0.30, 0.60) |
| (2-187) | (3-31-2) | | | | | | |
| Example | Compound | 4.4 | 13.3 | 5000.0 | 37.5 | 139.4 | (0.31, 0.61) |
| (2-188) | (3-32-2) | | | | | | |
| Example | Compound | 4.4 | 12.9 | 5000.0 | 38.6 | 126.3 | (0.31, 0.60) |
| (2-189) | (3-33-2) | | | | | | |
| Example | Compound | 4.5 | 13.7 | 5000.0 | 36.6 | 125.9 | (0.33, 0.61) |
| (2-190) | (3-34-2) | | | | | | |
| Example | Compound | 4.5 | 13.9 | 5000.0 | 35.9 | 141.5 | (0.32, 0.61) |
| (2-191) | (3-35-2) | | | | | | |
| Example | Compound | 4.5 | 13.6 | 5000.0 | 36.8 | 109.8 | (0.33, 0.60) |
| (2-192) | (3-36-2) | | | | | | |
| Example | Compound | 4.5 | 12.6 | 5000.0 | 39.6 | 116.5 | (0.32, 0.61) |
| (2-193) | (3-37-2) | | | | | | |
| Example | Compound | 4.5 | 14.2 | 5000.0 | 35.1 | 132.3 | (0.31, 0.60) |
| (2-194) | (3-38-2) | | | | | | |
| Example | Compound | 4.5 | 14.2 | 5000.0 | 35.2 | 112.9 | (0.31, 0.61) |
| (2-195) | (3-39-2) | | | | | | |
| Example | Compound | 4.4 | 13.8 | 5000.0 | 36.2 | 112.6 | (0.31, 0.60) |
| (2-196) | (3-40-2) | | | | | | |
| Example | Compound | 4.5 | 12.6 | 5000.0 | 39.6 | 142.4 | (0.33, 0.61) |
| (2-197) | (3-41-2) | | | | | | |
| Example | Compound | 4.4 | 13.8 | 5000.0 | 36.2 | 134.5 | (0.30, 0.60) |
| (2-198) | (3-42-2) | | | | | | |
| Example | Compound | 4.5 | 13.3 | 5000.0 | 37.5 | 99.2 | (0.31, 0.61) |
| (2-199) | (3-43-2) | | | | | | |
| Example | Compound | 4.4 | 13.0 | 5000.0 | 38.5 | 114.9 | (0.31, 0.60) |
| (2-200) | (3-44-2) | | | | | | |
| Example | Compound | 4.4 | 12.6 | 5000.0 | 39.7 | 130.2 | (0.33, 0.61) |
| (2-201) | (3-45-2) | | | | | | |
| Example | Compound | 4.5 | 12.5 | 5000.0 | 39.9 | 91.7 | (0.32, 0.61) |
| (2-202) | (3-46-2) | | | | | | |
| Example | Compound | 4.4 | 13.7 | 5000.0 | 36.4 | 142.1 | (0.33, 0.61) |
| (2-203) | (3-47-2) | | | | | | |
| Example | Compound | 4.5 | 13.4 | 5000.0 | 37.3 | 134.2 | (0.30, 0.60) |
| (2-204) | (3-48-2) | | | | | | |
| Example | Compound | 4.4 | 13.3 | 5000.0 | 37.7 | 98.0 | (0.32, 0.61) |
| (2-205) | (3-49-2) | | | | | | |
| Example | Compound | 4.4 | 12.5 | 5000.0 | 39.9 | 101.0 | (0.31, 0.60) |
| (2-206) | (3-50-2) | | | | | | |
| Example | Compound | 4.4 | 13.4 | 5000.0 | 37.2 | 127.0 | (0.31, 0.61) |
| (2-207) | (3-51-2) | | | | | | |
| Example | Compound | 4.5 | 13.5 | 5000.0 | 37.0 | 100.6 | (0.31, 0.60) |
| (2-208) | (3-52-2) | | | | | | |
| Example | Compound | 4.5 | 13.8 | 5000.0 | 36.2 | 103.6 | (0.33, 0.61) |
| (2-209) | (3-53-2) | | | | | | |
| Example | Compound | 4.4 | 13.8 | 5000.0 | 36.3 | 102.9 | (0.32, 0.61) |
| (2-210) | (3-54-2) | | | | | | |
| Example | Compound | 4.5 | 13.3 | 5000.0 | 37.5 | 117.5 | (0.33, 0.60) |
| (2-211) | (3-55-2) | | | | | | |
| Example | Compound | 4.4 | 14.3 | 5000.0 | 35.0 | 101.6 | (0.32, 0.61) |
| (2-212) | (3-56-2) | | | | | | |
| Example | Compound | 4.5 | 13.7 | 5000.0 | 36.5 | 118.4 | (0.31, 0.60) |
| (2-213) | (3-57-2) | | | | | | |
| Example | Compound | 4.5 | 12.7 | 5000.0 | 39.3 | 117.6 | (0.31, 0.61) |
| (2-214) | (3-58-2) | | | | | | |
| Example | Compound | 4.4 | 14.1 | 5000.0 | 35.5 | 124.0 | (0.31, 0.60) |
| (2-215) | (3-59-2) | | | | | | |
| Example | Compound | 4.4 | 14.1 | 5000.0 | 35.5 | 124.2 | (0.33, 0.61) |
| (2-216) | (3-60-2) | | | | | | |
| Example | Compound | 4.5 | 13.7 | 5000.0 | 36.4 | 137.3 | (0.30, 0.60) |
| (2-217) | (3-61-2) | | | | | | |
| Example | Compound | 4.4 | 12.9 | 5000.0 | 38.9 | 131.3 | (0.31, 0.61) |
| (2-218) | (3-62-2) | | | | | | |
| Example | Compound | 4.5 | 14.2 | 5000.0 | 35.2 | 108.5 | (0.31, 0.60) |
| (2-219) | (3-63-2) | | | | | | |
| Example | Compound | 4.4 | 13.9 | 5000.0 | 36.0 | 129.5 | (0.33, 0.61) |
| (2-220) | (3-64-2) | | | | | | |
| Example | Compound | 4.5 | 14.2 | 5000.0 | 35.1 | 145.5 | (0.32, 0.61) |
| (2-221) | (3-65-2) | | | | | | |
| Example | Compound | 4.5 | 12.7 | 5000.0 | 39.5 | 113.3 | (0.33, 0.60) |
| (2-222) | (3-66-2) | | | | | | |
| Example | Compound | 4.5 | 12.5 | 5000.0 | 40.0 | 112.8 | (0.32, 0.61) |
| (2-223) | (3-67-2) | | | | | | |
| Example | Compound | 4.4 | 12.5 | 5000.0 | 40.0 | 141.2 | (0.31, 0.60) |
| (2-224) | (3-68-2) | | | | | | |
| Example | Compound | 4.5 | 13.7 | 5000.0 | 36.5 | 112.5 | (0.31, 0.61) |
| (2-225) | (3-69-2) | | | | | | |
| Example | Compound | 4.5 | 13.5 | 5000.0 | 37.1 | 99.8 | (0.31, 0.60) |
| (2-226) | (3-70-2) | | | | | | |
| Example | Compound | 4.5 | 14.2 | 5000.0 | 35.3 | 132.3 | (0.33, 0.61) |
| (2-227) | (3-71-2) | | | | | | |
| Example | Compound | 4.4 | 13.8 | 5000.0 | 36.3 | 131.0 | (0.30, 0.60) |
| (2-228) | (3-72-2) | | | | | | |
| Example | Compound | 4.5 | 13.4 | 5000.0 | 37.3 | 102.0 | (0.31, 0.61) |
| (2-229) | (3-73-2) | | | | | | |
| Example | Compound | 4.5 | 12.6 | 5000.0 | 39.8 | 101.9 | (0.31, 0.60) |
| (2-230) | (3-74-2) | | | | | | |
| Example | Compound | 4.5 | 12.7 | 5000.0 | 39.4 | 123.5 | (0.33, 0.61) |
| (2-231) | (3-75-2) | | | | | | |
| Example | Compound | 4.5 | 13.1 | 5000.0 | 38.2 | 113.1 | (0.32, 0.61) |
| (2-232) | (3-76-2) | | | | | | |
| Example | Compound | 4.5 | 13.9 | 5000.0 | 35.9 | 100.1 | (0.33, 0.60) |
| (2-233) | (3-77-2) | | | | | | |
| Example | Compound | 4.5 | 13.2 | 5000.0 | 37.8 | 98.2 | (0.32, 0.61) |
| (2-234) | (3-78-2) | | | | | | |
| Example | Compound | 4.4 | 14.1 | 5000.0 | 35.4 | 115.9 | (0.31, 0.60) |
| (2-235) | (3-79-2) | | | | | | |
| Example | Compound | 4.4 | 13.9 | 5000.0 | 36.0 | 99.0 | (0.33, 0.61) |
| (2-236) | (3-80-2) | | | | | | |
| Example | Compound | 4.4 | 14.0 | 5000.0 | 35.8 | 94.2 | (0.30, 0.60) |
| (2-237) | (3-81-2) | | | | | | |
| Example | Compound | 4.5 | 12.5 | 5000.0 | 39.9 | 127.3 | (0.31, 0.61) |
| (2-238) | (3-82-2) | | | | | | |
| Example | Compound | 4.4 | 14.3 | 5000.0 | 35.0 | 131.4 | (0.31, 0.60) |
| (2-239) | (3-83-2) | | | | | | |
| Example | Compound | 4.4 | 13.1 | 5000.0 | 38.2 | 98.9 | (0.33, 0.61) |
| (2-240) | (3-84-2) | | | | | | |
| Example | Compound | 4.4 | 12.9 | 5000.0 | 38.6 | 91.3 | (0.32, 0.61) |
| (2-241) | (3-85-2) | | | | | | |
| Example | Compound | 4.4 | 13.9 | 5000.0 | 35.9 | 99.7 | (0.33, 0.60) |
| (2-242) | (3-86-2) | | | | | | |
| Example | Compound | 4.4 | 12.5 | 5000.0 | 39.9 | 141.9 | (0.32, 0.61) |
| (2-243) | (3-87-2) | | | | | | |
| Example | Compound | 4.5 | 13.1 | 5000.0 | 38.1 | 91.8 | (0.31, 0.60) |
| (2-244) | (3-88-2) | | | | | | |
| Example | Compound | 4.4 | 12.5 | 5000.0 | 40.0 | 121.9 | (0.31, 0.61) |
| (2-245) | (3-89-2) | | | | | | |
| Example | Compound | 4.5 | 12.6 | 5000.0 | 39.7 | 148.2 | (0.31, 0.60) |
| (2-246) | (3-90-2) | | | | | | |
| Example | Compound | 4.5 | 12.9 | 5000.0 | 38.9 | 124.0 | (0.33, 0.61) |
| (2-247) | (3-91-2) | | | | | | |
| Example | Compound | 4.4 | 14.2 | 5000.0 | 35.3 | 92.0 | (0.30, 0.60) |
| (2-248) | (3-92-2) | | | | | | |
| Example | Compound | 4.5 | 13.4 | 5000.0 | 37.3 | 106.3 | (0.31, 0.61) |
| (2-249) | (3-93-2) | | | | | | |
| Example | Compound | 4.5 | 12.9 | 5000.0 | 38.8 | 111.3 | (0.31, 0.60) |
| (2-250) | (3-94-2) | | | | | | |
| Example | Compound | 4.5 | 13.1 | 5000.0 | 38.2 | 149.7 | (0.33, 0.61) |
| (2-251) | (3-95-2) | | | | | | |
| Example | Compound | 4.4 | 12.7 | 5000.0 | 39.2 | 116.4 | (0.32, 0.61) |
| (2-252) | (3-96-2) | | | | | | |
| Example | Compound | 4.5 | 12.8 | 5000.0 | 39.1 | 112.7 | (0.33, 0.61) |
| (2-253) | (3-97-2) | | | | | | |
| Example | Compound | 4.4 | 14.2 | 5000.0 | 35.3 | 120.3 | (0.30, 0.60) |
| (2-254) | (3-98-2) | | | | | | |
| Example | Compound | 4.5 | 14.0 | 5000.0 | 35.8 | 148.6 | (0.32, 0.61) |
| (2-255) | (3-99-2) | | | | | | |
| Example | Compound | 4.4 | 14.1 | 5000.0 | 35.4 | 94.8 | (0.31, 0.60) |
| (2-256) | (3-100-2) | | | | | | |
| Example | Compound | 4.5 | 12.9 | 5000.0 | 38.7 | 126.7 | (0.30, 0.60) |
| (2-257) | (3-101-2) | | | | | | |
| Example | Compound | 4.5 | 12.6 | 5000.0 | 39.8 | 126.5 | (0.31, 0.61) |
| (2-258) | (3-102-2) | | | | | | |
| Example | Compound | 4.5 | 14.2 | 5000.0 | 35.2 | 103.6 | (0.31, 0.60) |
| (2-259) | (3-103-2) | | | | | | |
| Example | Compound | 4.4 | 14.2 | 5000.0 | 35.3 | 105.3 | (0.33, 0.61) |
| (2-260) | (3-104-2) | | | | | | |
| Example | Compound | 4.4 | 14.0 | 5000.0 | 35.6 | 93.0 | (0.32, 0.61) |
| (2-261) | (3-105-2) | | | | | | |
| Example | Compound | 4.5 | 12.6 | 5000.0 | 39.7 | 116.9 | (0.33, 0.60) |
| (2-262) | (3-106-2) | | | | | | |
| Example | Compound | 4.5 | 14.2 | 5000.0 | 35.2 | 125.5 | (0.32, 0.61) |
| (2-263) | (3-107-2) | | | | | | |
| Example | Compound | 4.5 | 13.4 | 5000.0 | 37.4 | 111.9 | (0.31, 0.60) |
| (2-264) | (3-108-2) | | | | | | |
| Example | Compound | 4.4 | 13.2 | 5000.0 | 38.0 | 116.4 | (0.31, 0.61) |
| (2-265) | (3-109-2) | | | | | | |
| Example | Compound | 4.4 | 13.3 | 5000.0 | 37.7 | 114.3 | (0.31, 0.60) |
| (2-266) | (3-110-2) | | | | | | |
| Example | Compound | 4.5 | 13.4 | 5000.0 | 37.3 | 111.5 | (0.33, 0.61) |
| (2-267) | (3-111-2) | | | | | | |
| Example | Compound | 4.4 | 14.2 | 5000.0 | 35.1 | 141.7 | (0.30, 0.60) |
| (2-268) | (3-112-2) | | | | | | |
| Example | Compound | 4.5 | 13.9 | 5000.0 | 35.9 | 127.4 | (0.31, 0.61) |
| (2-269) | (3-113-2) | | | | | | |
| Example | Compound | 4.5 | 12.8 | 5000.0 | 39.0 | 150.0 | (0.31, 0.60) |
| (2-270) | (3-114-2) | | | | | | |
| Example | Compound | 4.5 | 13.1 | 5000.0 | 38.2 | 95.2 | (0.33, 0.61) |
| (2-271) | (3-115-2) | | | | | | |
| Example | Compound | 4.4 | 13.7 | 5000.0 | 36.5 | 145.9 | (0.32, 0.61) |
| (2-272) | (3-116-2) | | | | | | |
| Example | Compound | 4.5 | 12.8 | 5000.0 | 39.0 | 120.4 | (0.33, 0.60) |
| (2-273) | (3-117-2) | | | | | | |
| Example | Compound | 4.4 | 13.3 | 5000.0 | 37.5 | 146.0 | (0.32, 0.61) |
| (2-274) | (3-118-2) | | | | | | |
| Example | Compound | 4.4 | 14.0 | 5000.0 | 35.6 | 110.0 | (0.31, 0.60) |
| (2-275) | (3-119-2) | | | | | | |
| Example | Compound | 4.4 | 13.6 | 5000.0 | 36.7 | 134.5 | (0.31, 0.61) |
| (2-276) | (3-120-2) | | | | | | |
| Example | Compound | 4.5 | 13.8 | 5000.0 | 36.1 | 139.5 | (0.31, 0.60) |
| (2-277) | (3-121-2) | | | | | | |
| Example | Compound | 4.4 | 12.6 | 5000.0 | 39.8 | 102.9 | (0.33, 0.61) |
| (2-278) | (3-122-2) | | | | | | |
| Example | Compound | 4.5 | 14.1 | 5000.0 | 35.5 | 112.8 | (0.30, 0.60) |
| (2-279) | (3-123-2) | | | | | | |
| Example | Compound | 4.5 | 13.8 | 5000.0 | 36.3 | 100.6 | (0.31, 0.61) |
| (2-280) | (3-124-2) | | | | | | |
| Example | Compound | 4.5 | 13.0 | 5000.0 | 38.4 | 90.7 | (0.31, 0.60) |
| (2-281) | (3-125-2) | | | | | | |
| Example | Compound | 4.5 | 12.9 | 5000.0 | 38.7 | 144.9 | (0.33, 0.61) |
| (2-282) | (3-126-2) | | | | | | |
| Example | Compound | 4.4 | 13.3 | 5000.0 | 37.7 | 105.1 | (0.32, 0.61) |
| (2-283) | (3-127-2) | | | | | | |
| Example | Compound | 4.5 | 13.5 | 5000.0 | 37.1 | 137.4 | (0.33, 0.60) |
| (2-284) | (3-128-2) | | | | | | |
| Example | Compound | 4.7 | 13.2 | 5000.0 | 37.8 | 123.3 | (0.31, 0.61) |
| (2-285) | (4-1-2) | | | | | | |
| Example | Compound | 5.0 | 13.4 | 5000.0 | 37.4 | 141.1 | (0.31, 0.60) |
| (2-286) | (4-2-2) | | | | | | |
| Example | Compound | 4.6 | 16.0 | 5000.0 | 31.2 | 110.6 | (0.33, 0.61) |
| (2-287) | (4-3-2) | | | | | | |
| Example | Compound | 4.6 | 13.6 | 5000.0 | 36.8 | 145.2 | (0.32, 0.61) |
| (2-288) | (4-4-2) | | | | | | |
| Example | Compound | 4.6 | 12.9 | 5000.0 | 38.6 | 138.5 | (0.33, 0.60) |
| (2-289) | (4-5-2) | | | | | | |
| Example | Compound | 4.6 | 15.0 | 5000.0 | 33.4 | 147.5 | (0.32, 0.61) |
| (2-290) | (4-6-2) | | | | | | |
| Example | Compound | 4.8 | 13.1 | 5000.0 | 38.1 | 114.2 | (0.31, 0.60) |
| (2-291) | (4-7-2) | | | | | | |
| Example | Compound | 4.6 | 14.9 | 5000.0 | 33.7 | 128.3 | (0.31, 0.61) |
| (2-292) | (4-8-2) | | | | | | |
| Example | Compound | 4.9 | 13.4 | 5000.0 | 37.3 | 135.6 | (0.31, 0.60) |
| (2-293) | (4-9-2) | | | | | | |
| Example | Compound | 4.6 | 14.1 | 5000.0 | 35.4 | 143.6 | (0.33, 0.61) |
| (2-294) | (4-10-2) | | | | | | |
| Example | Compound | 5.0 | 14.7 | 5000.0 | 34.0 | 98.8 | (0.30, 0.60) |
| (2-295) | (4-11-2) | | | | | | |
| Example | Compound | 4.8 | 12.7 | 5000.0 | 39.5 | 102.2 | (0.31, 0.61) |
| (2-296) | (4-12-2) | | | | | | |
| Example | Compound | 4.9 | 14.2 | 5000.0 | 35.3 | 109.8 | (0.31, 0.60) |
| (2-297) | (4-13-2) | | | | | | |
| Example | Compound | 4.5 | 15.4 | 5000.0 | 32.4 | 120.5 | (0.33, 0.61) |
| (2-298) | (4-14-2) | | | | | | |
| Example | Compound | 4.9 | 14.2 | 5000.0 | 35.1 | 129.8 | (0.32, 0.61) |
| (2-299) | (4-15-2) | | | | | | |
| Example | Compound | 4.9 | 15.2 | 5000.0 | 33.0 | 91.6 | (0.33, 0.60) |
| (2-300) | (4-16-2) | | | | | | |
| Example | Compound | 5.0 | 14.9 | 5000.0 | 33.7 | 94.7 | (0.32, 0.61) |
| (2-301) | (4-17-2) | | | | | | |
| Example | Compound | 4.6 | 12.8 | 5000.0 | 39.1 | 96.2 | (0.31, 0.60) |
| (2-302) | (4-18-2) | | | | | | |
| Example | Compound | 4.5 | 13.9 | 5000.0 | 36.0 | 95.1 | (0.31, 0.61) |
| (2-303) | (4-19-2) | | | | | | |
| Example | Compound | 4.7 | 12.7 | 5000.0 | 39.3 | 111.7 | (0.31, 0.60) |
| (2-304) | (4-20-2) | | | | | | |
| Example | Compound | 5.0 | 16.4 | 5000.0 | 30.4 | 121.8 | (0.33, 0.61) |
| (2-305) | (4-21-2) | | | | | | |
| Example | Compound | 4.5 | 14.0 | 5000.0 | 35.6 | 128.2 | (0.30, 0.60) |
| (2-306) | (4-22-2) | | | | | | |
| Example | Compound | 4.8 | 15.0 | 5000.0 | 33.4 | 103.7 | (0.31, 0.61) |
| (2-307) | (4-23-2) | | | | | | |
| Example | Compound | 4.9 | 13.4 | 5000.0 | 37.2 | 99.4 | (0.31, 0.60) |
| (2-308) | (4-24-2) | | | | | | |
| Example | Compound | 4.9 | 13.6 | 5000.0 | 36.8 | 131.8 | (0.33, 0.61) |
| (2-309) | (4-25-2) | | | | | | |
| Example | Compound | 4.6 | 13.5 | 5000.0 | 37.1 | 108.3 | (0.32, 0.61) |
| (2-310) | (4-26-2) | | | | | | |
| Example | Compound | 4.8 | 13.4 | 5000.0 | 37.3 | 97.2 | (0.33, 0.60) |
| (2-311) | (4-27-2) | | | | | | |
| Example | Compound | 4.5 | 14.5 | 5000.0 | 34.5 | 94.8 | (0.32, 0.61) |
| (2-312) | (4-28-2) | | | | | | |

### II. Manufacture and Test of Red Organic Light Emitting Element (Phosphorescent Host)

### Example 2-313 Red Organic Light Emitting Element (Phosphorescent Host)

An organic electronic light emitting element was manufactured by an ordinary method using the compound obtained through synthesis as a light emitting host material for a light emitting layer. First, a film of N¹-(naphthalen-2-yl)-N⁴,N⁴-bis(4-(naphthalen-2-yl(phenyl)amino)phenyl)-N¹-phenylbenzene-1,4-diamine (hereinafter, abbreviated as “2-TNATA”) was vacuum-deposited on an ITO layer (anode) formed on a galas substrate to form a hole injection layer with a thickness 60 nm, and then, 4,4-bis[N-(1-naphthyl)-N-phenylamino]biphenyl (hereinafter, abbreviated as “-NPD”) as a hole transport compound was vacuum-deposited on the hole injection layer to form a hole transport layer with a thickness of 60 nm. Then, a light emitting layer with a thickness of 30 nm was deposited on the hole transport layer by doping an upper portion of the hole transport layer with compound 2-41-2 of the present invention as a host material and (piq)₂Ir(acac) [bis-(1-phenylisoquinolyl)iridium(III)acetylacetonate] as a dopant material at a weight ratio of 95:5. Then, (1.1′-bisphenyl)-4-olato)bis(2-methyl-8-quinolinolato)aluminum (hereinafter, abbreviated as “BAlq”) was vacuum-deposited with a thickness of 10 nm for a hole blocking layer, and tris(8-quinolinol)aluminum (hereinafter, abbreviated as “Alq3”) was formed with a thickness of 40 nm for an electron transport layer. Thereafter, LiF as halogenated alkali metal was deposited with a thickness of 0.2 nm for an electron injection layer, and then Al was deposited with a thickness of 150 nm to be used as a cathode. In this way, an organic electronic light emitting element was manufactured.

### Example 2-314 to Example 2-336 Red Organic Light Emitting Element (Phosphorescent Host)

An organic electronic light emitting element was manufactured by the same method as in Example 2-313 except that, instead of compound 2-41-2 of the present invention, one of compounds 2-42-2 to 2-52-2 and 3-41-2 to 3-52-2 listed on table 6 was used as a phosphorescent host material for a light emitting layer.

### Comparative Example 2-5

An organic electronic light emitting element was manufactured by the same method as in Example 2-313 except that, instead of compound 2-41-2 of the present invention, comparative compound A [4,4′-N,N′-dicarbazole-biphenyl (CBP)] above was used as a phosphorescent host material for a light emitting layer.

### Comparative Example 2-6

An organic electronic light emitting element was manufactured by the same method as in Example 2-313 except that, instead of compound 2-41-2 of the present invention, comparative compound B above was used as a phosphorescent host material for a light emitting layer.

### Comparative Example 2-7

An organic electronic light emitting element was manufactured by the same method as in Example 2-313 except that, instead of compound 2-41-2 of the present invention, comparative compound C above was used as a phosphorescent host material for a light emitting layer.

### Comparative Example 2-8

An organic electronic light emitting element was manufactured by the same method as in Example 2-313 except that, instead of compound 2-41-2 of the present invention, comparative compound D above was used as a phosphorescent host material for a light emitting layer.

A forward bias DC voltage was applied to the organic electronic light emitting elements manufactured in Examples 2-313 to 2-336 and Comparative Examples 2-5 to 2-8 to measure electro-luminescence (EL) characteristics thereof by PR-650 (Photoresearch), and the T95 lifetime was measured by lifetime measuring equipments (Mcscience) at reference brightness of 2500 cd/m². Table 2-6 below shows the manufacture of elements and evaluation results thereof.

TABLE 2-6

| | | | Current | Brightness | | Lifetime | CIE |
|---|---|---|---|---|---|---|---|
| | Compound | Voltage | Density | (cd/m2) | Efficiency | T(95) | (y, x) |
| Comparative | Compound | 6.2 | 39.7 | 2500.0 | 6.3 | 53.3 | (0.31, |
| Example (2-5) | (A) | | | | | | 0.60) |
| Comparative | Compound | 5.7 | 32.5 | 2500.0 | 7.7 | 87.2 | (0.31, |
| Example (2-6) | (B) | | | | | | 0.61) |
| Comparative | Compound | 5.8 | 34.8 | 2500.0 | 7.2 | 81.8 | (0.31, |
| Example (2-7) | (C) | | | | | | 0.60) |
| Comparative | Compound | 5.9 | 34.7 | 2500.0 | 7.2 | 83.3 | (0.33, |
| Example (2-8) | (D) | | | | | | 0.61) |
| Example (2-313) | Compound | 5.2 | 24.2 | 2500.0 | 10.3 | 110.9 | (0.30, |
| | (2-41-2) | | | | | | 0.60) |
| Example (2-314) | Compound | 5.3 | 27.3 | 2500.0 | 9.1 | 145.3 | (0.31, |
| | (2-42-2) | | | | | | 0.61) |
| Example (2-315) | Compound | 5.2 | 26.4 | 2500.0 | 9.5 | 118.9 | (0.31, |
| | (2-43-2) | | | | | | 0.60) |
| Example (2-316) | Compound | 5.2 | 25.4 | 2500.0 | 9.9 | 114.8 | (0.33, |
| | (2-44-2) | | | | | | 0.61) |
| Example (2-317) | Compound | 5.1 | 29.2 | 2500.0 | 8.6 | 142.1 | (0.32, |
| | (2-45-2) | | | | | | 0.61) |
| Example (2-318) | Compound | 5.2 | 19.8 | 2500.0 | 12.6 | 141.0 | (0.33, |
| | (2-46-2) | | | | | | 0.60) |
| Example (2-319) | Compound | 5.0 | 24.4 | 2500.0 | 10.3 | 96.9 | (0.32, |
| | (2-47-2) | | | | | | 0.61) |
| Example (2-320) | Compound | 5.2 | 19.9 | 2500.0 | 12.6 | 141.2 | (0.31, |
| | (2-48-2) | | | | | | 0.60) |
| Example (2-321) | Compound | 5.3 | 31.0 | 2500.0 | 8.1 | 97.8 | (0.31, |
| | (2-49-2) | | | | | | 0.61) |
| Example (2-322) | Compound | 5.2 | 20.1 | 2500.0 | 12.4 | 140.1 | (0.31, |
| | (2-50-2) | | | | | | 0.60) |
| Example (2-323) | Compound | 5.1 | 21.7 | 2500.0 | 11.5 | 134.1 | (0.33, |
| | (2-51-2) | | | | | | 0.61) |
| Example (2-324) | Compound | 5.3 | 21.2 | 2500.0 | 11.8 | 130.7 | (0.30, |
| | (2-52-2) | | | | | | 0.60) |
| Example (2-325) | Compound | 5.0 | 20.0 | 2500.0 | 12.5 | 107.3 | (0.31, |
| | (3-41-2) | | | | | | 0.61) |
| Example (2-326) | Compound | 5.1 | 20.3 | 2500.0 | 12.3 | 105.4 | (0.31, |
| | (3-42-2) | | | | | | 0.60) |
| Example (2-327) | Compound | 5.0 | 20.1 | 2500.0 | 12.4 | 97.9 | (0.33, |
| | (3-43-2) | | | | | | 0.61) |
| Example (2-328) | Compound | 5.1 | 18.9 | 2500.0 | 13.2 | 109.5 | (0.32, |
| | (3-44-2) | | | | | | 0.61) |
| Example (2-329) | Compound | 5.0 | 17.0 | 2500.0 | 14.7 | 146.6 | (0.33, |
| | (3-45-2) | | | | | | 0.60) |
| Example (2-330) | Compound | 5.0 | 17.9 | 2500.0 | 13.9 | 126.5 | (0.31, |
| | (3-46-2) | | | | | | 0.60) |
| Example (2-331) | Compound | 5.0 | 20.0 | 2500.0 | 12.5 | 128.0 | (0.31, |
| | (3-47-2) | | | | | | 0.61) |
| Example (2-332) | Compound | 5.0 | 19.4 | 2500.0 | 12.9 | 91.1 | (0.31, |
| | (3-48-2) | | | | | | 0.60) |
| Example (2-333) | Compound | 5.0 | 19.5 | 2500.0 | 12.8 | 132.7 | (0.33, |
| | (3-49-2) | | | | | | 0.61) |
| Example (2-334) | Compound | 5.0 | 19.8 | 2500.0 | 12.6 | 110.3 | (0.30, |
| | (3-50-2) | | | | | | 0.60) |
| Example (2-335) | Compound | 5.1 | 17.8 | 2500.0 | 14.0 | 147.9 | (0.31, |
| | (3-51-2) | | | | | | 0.61) |
| Example (2-336) | Compound | 5.0 | 20.7 | 2500.0 | 12.1 | 131.1 | (0.31, |
| | (3-52-2) | | | | | | 0.60) |

As can be seen from the results on table 2-5 and table 2-6, the organic electronic light emitting elements using the materials for the organic electronic light emitting element of the present invention as a phosphorescent host showed a low driving voltage, high light emitting efficiency, and a long lifetime.

In other words, comparative compounds B, C, and D having bis-carbazole as a core showed excellent element results compared with comparative compound A, which is CBP generally used as a host material, and the compounds of the present invention having carbazole linked to carboline showed the best results in view of a driving voltage, efficiency, and a lifetime, compared with comparative compounds B, C, and D.

The compound according to the present invention has a bipolar since it is composed of carbazole and carboline. Therefore, it is considered that the compounds of the present invention can raise the charge balance in the light emitting layer compared with those in comparative compounds B, C, and D, leading to an increase in efficiency, and shows less hole accumulation in the light emitting layer compared with comparative compounds B, C, and D, leading to a long lifetime (In the driving of OLED, holes generally have 1000-fold higher mobility than electrons).

In addition, the compounds according to the present invention have similar T1 values to comparative compounds B, C, and D, but show lower LUMO values, and resultantly, it is considered that the compounds of the present invention may easily receive electrons from the electron transport layer, leading to a low driving voltage and excellent thermal stability (thermal damage due to a high driving voltage).

In addition, the characteristics of elements have been described in view of a light emitting layer from the foregoing evaluation results of the manufacture of elements, but the materials ordinarily used for a light emitting layer may be used alone or in a mixture with other materials, for the foregoing organic material layer for an organic electronic element, such as an an electron transport layer, an electron injection layer, a hole injection layer, a hole transport layer, and an auxiliary light emitting layer. Therefore, for the foregoing reasons, the compounds of the present invention may be used alone or in a mixture with other materials, for the other layers for the organic material layer excluding the light emitting layer, for example, an electron transport layer, an electron injection layer, a hole injection layer, a hole transport layer, and an auxiliary light emitting layer.

### Example 3

The compound according to an aspect of the present invention is represented by Formula 3-1 below.

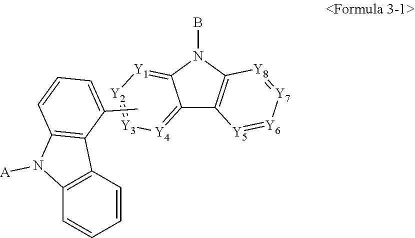

In Formula 3-1, A and B each may be independently selected from the group consisting of a C₆-C₆₀ aryl group, a fluorenyl group, a C₂-C₆₀ heterocyclic group containing at least one heteroatom of O, N, S, Si, and P, a fused ring group of a C₃-C₆o aliphatic group and a C₆-C₆₀ aromatic group, a C₁-C₅₀ alkyl group, a C₂-C₂₀ alkenyl group, a C₂-C₂₀ alkynyl group, C₁-C₃₀ alkoxyl group, a C₆-C₃₀ aryloxy group, and -L′-N(Rₐ) (R_{b}).

L′ may be selected from the group consisting of a single bond, a C₆-C₆₀ arylene group, a fluorenyl group, a fused ring group of a C₃-C₆₀ aliphatic group and a C₆-C₆₀ aromatic group, and a C₂-C₆₀ heterocyclic group.

Rₐ and R_{b} each may be independently selected from the group consisting of a C₆-C₆₀ aryl group, a fluorenylene group, a fused ring group of a C₃-C₆o aliphatic group and a C₆-C₆₀ aromatic group, and a C₂-C₆₀ heterocyclic group containing at least one heteroatom of O, N, S, Si, and P.

Y₁ to Ye each may be independently CR or N, and at least one of Y₁ to Ye may be N.

At least one of R's may be linked to adjacent carbazole, and R that is not linked thereto may be hydrogen.

For example, when A, B, L′, Rₐ, and R_{b} are an aryl group, A, B, L′, Rₐ, and R_{b} each may be independently a phenyl group, a biphenyl group, a naphthyl group, or the like.

the aryl group, fluorenyl group, heterocyclic group, fused ring group, alkyl group, alkenyl group, alkoxyl group, aryloxy group, arylene group, and fluorenylene group each may be substituted with at least one substituent selected from the group consisting of deuterium, halogen, a silane group, a siloxane group, a boron group, a germanium group, a cyano group, a nitro group, a C₁-C₂₀ alkylthio group, a C₁-C₂₀ alkoxyl group, a C₁-C₂₀ alkyl group, a C₂-C₂₀ alkenyl group, a C₂-C₂₀ alkynyl group, a C₆-C₂₀ aryl group, a C₆-C₂₀ aryl group substituted with deuterium, a fluorenyl group, a C₂-C₂₀ heterocyclic group, a C₃-C₂₀ cycloalkyl group, a C₇-C₂₀ arylalkyl group, and a C₈-C₂₀ arylalkenyl group.

Here, the aryl group may be an aryl group having 6-60 carbon atoms, preferably 6-40 carbon atoms, and more preferably 6-30 carbon atoms;

the heterocyclic group may be a heterocyclic group having 2-60 carbon atoms, preferably 2-30 carbon atoms, and more preferably 2-20 carbon atoms;

the arylene group may be an arylene group having 6-60 carbon atoms, preferably 6-30 carbon atoms, and more preferably 6-20 carbon atoms; and

the alkyl group may be an alkyl group having 1-50 carbon atoms, preferably 1-30 carbon atoms, more preferably 1-20 carbon atoms, and especially preferably 1-10 carbon atoms.

Specifically, the compound represented by Formula 3-1 above may be expressed by one of the following compounds.

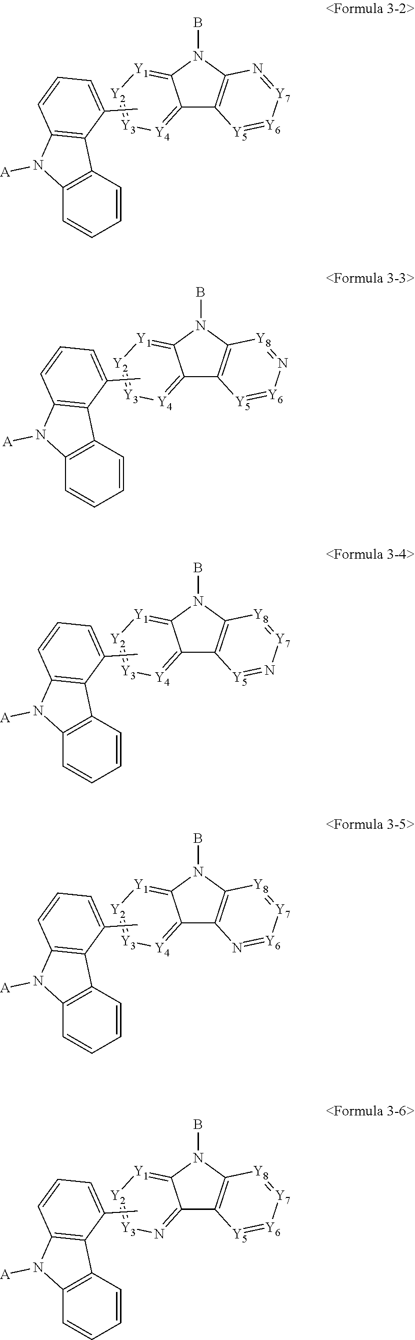

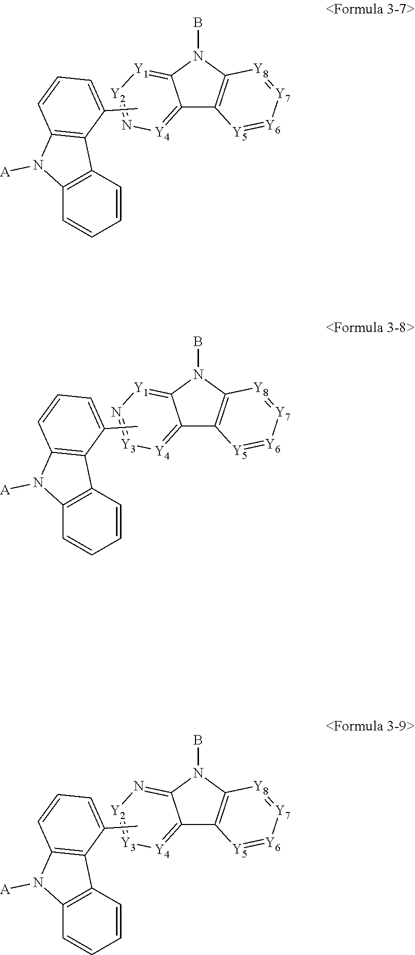

In Formulas 3-2 to 3-9,

Y₁ to Y₈ and A and B may be identical Y₁ to Y₈ and A and B defined in Formula 3-1.

More specifically, the compounds represented by Formula 3-1 may be one of the following compounds.

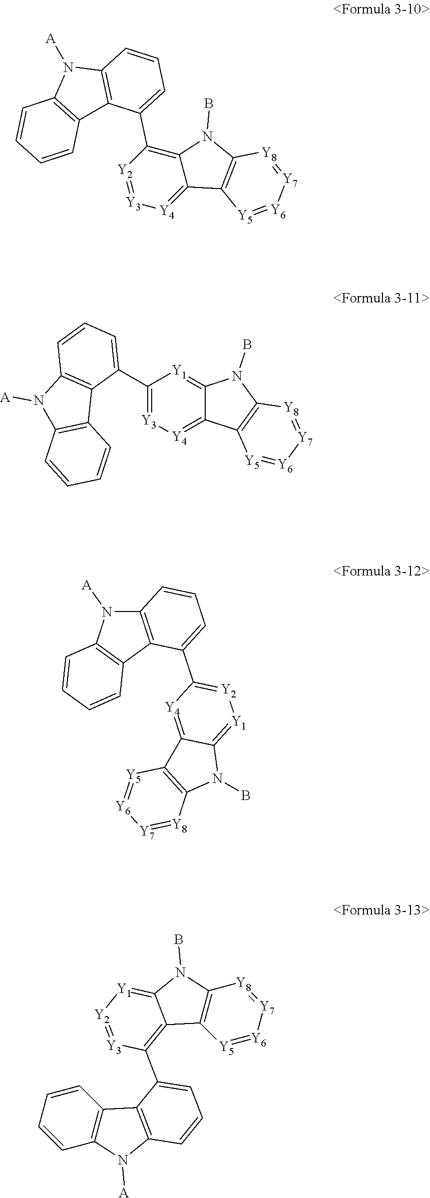

In Formulas 3-10 to 3-13,

Y₁ to Y₈ each may be independently CH or N, and at least one thereof is N, and A and B may be identical A and B defined in Formula 3-1.

More specifically, the compounds represented by Formulas 3-1 to 3-13 may be one of the following compounds.

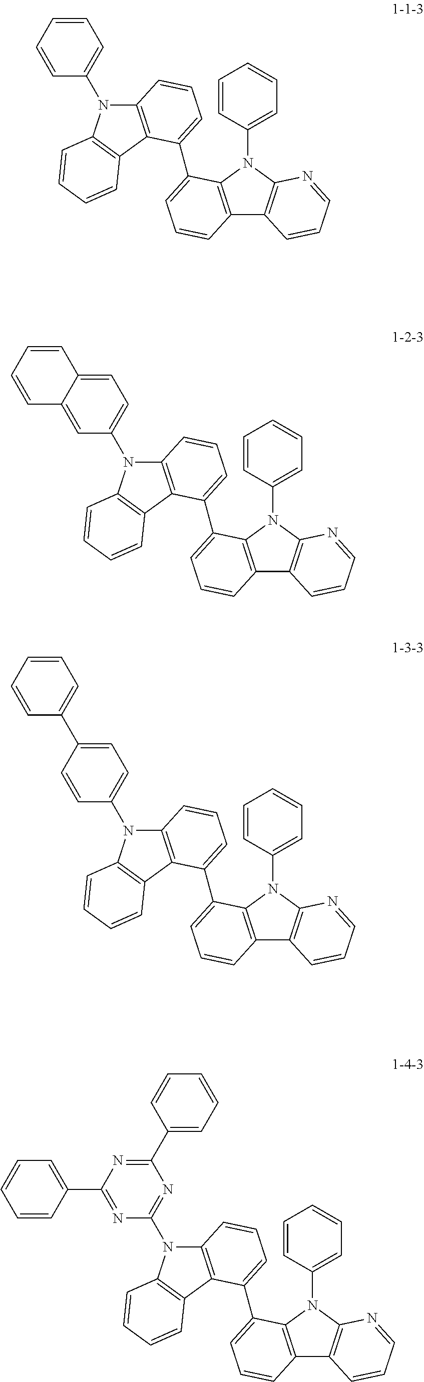 [SMILES: c1ccc(-n2c3ccccc3c3c(-c4cccc5c6cccnc6n(-c6ccccc6)c45)cccc32)cc1 ; c1ccc(-n2c3ncccc3c3cccc(-c4cccc5c4c4ccccc4n5-c4ccc5ccccc5c4)c32)cc1 ; c1ccc(-c2ccc(-n3c4ccccc4c4c(-c5cccc6c7cccnc7n(-c7ccccc7)c56)cccc43)cc2)cc1 ; c1ccc(-c2nc(-c3ccccc3)nc(-n3c4ccccc4c4c(-c5cccc6c7cccnc7n(-c7ccccc7)c56)cccc43)n2)cc1]

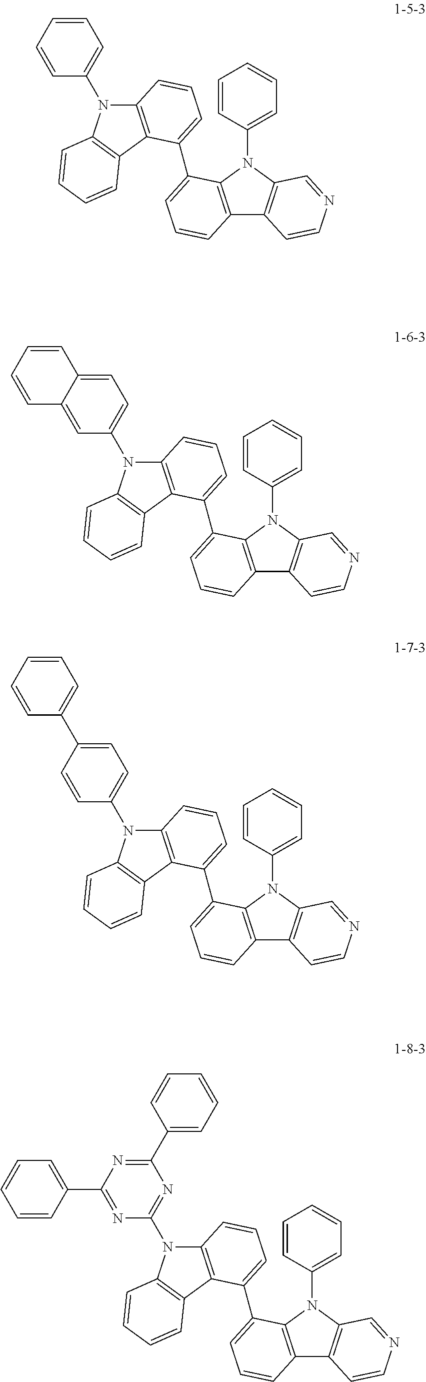 [SMILES: c1ccc(-n2c3ccccc3c3c(-c4cccc5c6ccncc6n(-c6ccccc6)c45)cccc32)cc1 ; c1ccc(-n2c3cnccc3c3cccc(-c4cccc5c4c4ccccc4n5-c4ccc5ccccc5c4)c32)cc1 ; c1ccc(-c2ccc(-n3c4ccccc4c4c(-c5cccc6c7ccncc7n(-c7ccccc7)c56)cccc43)cc2)cc1 ; c1ccc(-c2nc(-c3ccccc3)nc(-n3c4ccccc4c4c(-c5cccc6c7ccncc7n(-c7ccccc7)c56)cccc43)n2)cc1]

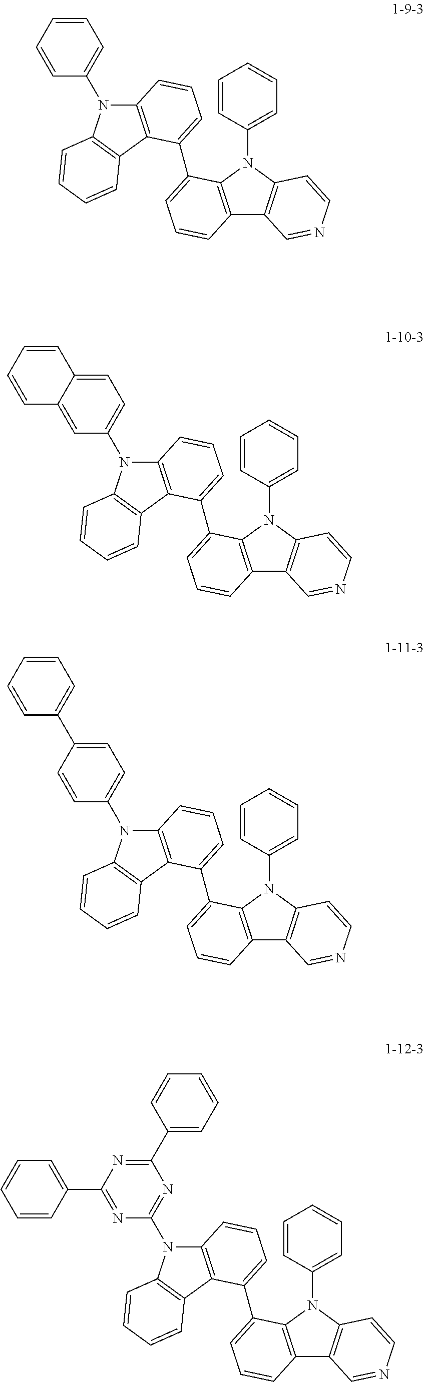 [SMILES: c1ccc(-n2c3ccccc3c3c(-c4cccc5c6cnccc6n(-c6ccccc6)c45)cccc32)cc1 ; c1ccc(-n2c3ccncc3c3cccc(-c4cccc5c4c4ccccc4n5-c4ccc5ccccc5c4)c32)cc1 ; c1ccc(-c2ccc(-n3c4ccccc4c4c(-c5cccc6c7cnccc7n(-c7ccccc7)c56)cccc43)cc2)cc1 ; c1ccc(-c2nc(-c3ccccc3)nc(-n3c4ccccc4c4c(-c5cccc6c7cnccc7n(-c7ccccc7)c56)cccc43)n2)cc1]

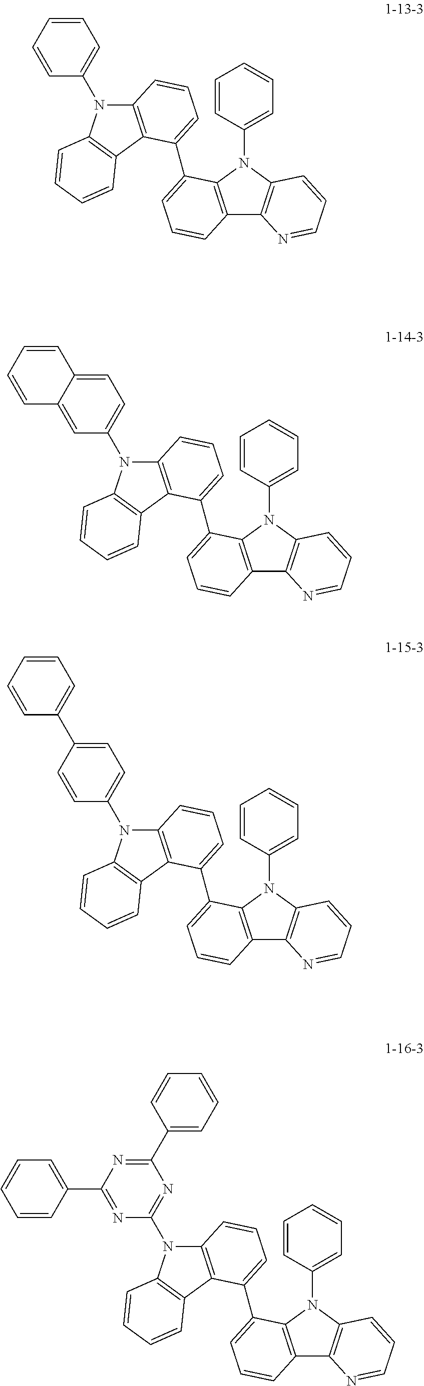 [SMILES: c1ccc(-n2c3ccccc3c3c(-c4cccc5c6ncccc6n(-c6ccccc6)c45)cccc32)cc1 ; c1ccc(-n2c3cccnc3c3cccc(-c4cccc5c4c4ccccc4n5-c4ccc5ccccc5c4)c32)cc1 ; c1ccc(-c2ccc(-n3c4ccccc4c4c(-c5cccc6c7ncccc7n(-c7ccccc7)c56)cccc43)cc2)cc1 ; c1ccc(-c2nc(-c3ccccc3)nc(-n3c4ccccc4c4c(-c5cccc6c7ncccc7n(-c7ccccc7)c56)cccc43)n2)cc1]

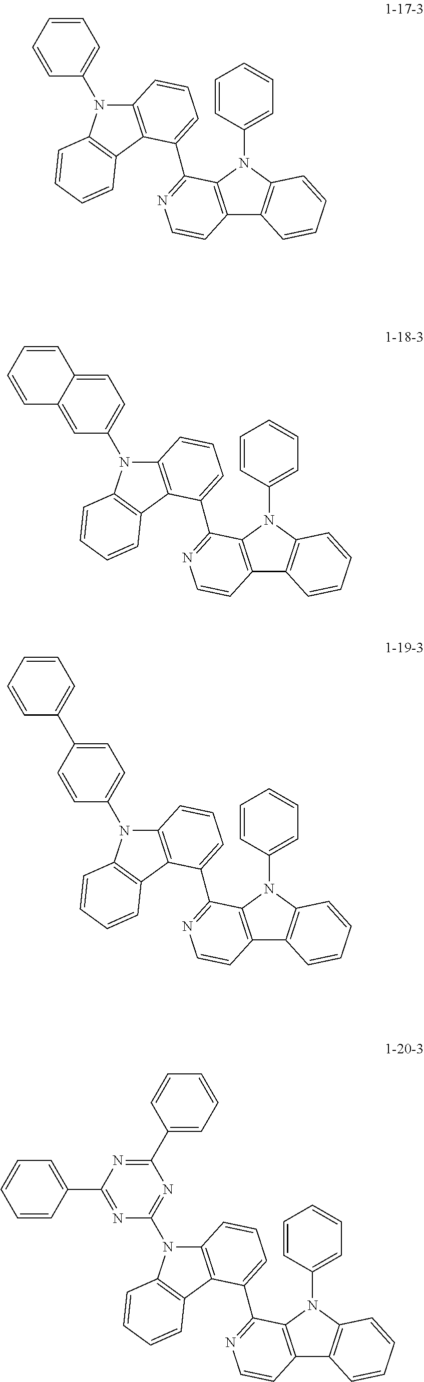 [SMILES: c1ccc(-n2c3ccccc3c3c(-c4nccc5c6ccccc6n(-c6ccccc6)c45)cccc32)cc1 ; c1ccc(-n2c3ccccc3c3ccnc(-c4cccc5c4c4ccccc4n5-c4ccc5ccccc5c4)c32)cc1 ; c1ccc(-c2ccc(-n3c4ccccc4c4c(-c5nccc6c7ccccc7n(-c7ccccc7)c56)cccc43)cc2)cc1 ; c1ccc(-c2nc(-c3ccccc3)nc(-n3c4ccccc4c4c(-c5nccc6c7ccccc7n(-c7ccccc7)c56)cccc43)n2)cc1]

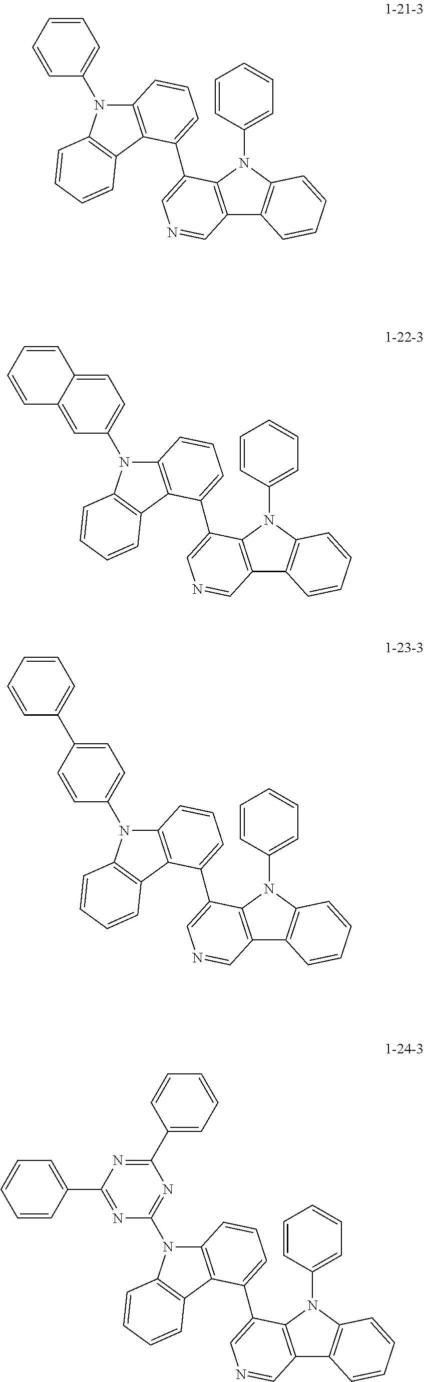 [SMILES: c1ccc(-n2c3ccccc3c3c(-c4cncc5c6ccccc6n(-c6ccccc6)c45)cccc32)cc1 ; c1ccc(-n2c3ccccc3c3cncc(-c4cccc5c4c4ccccc4n5-c4ccc5ccccc5c4)c32)cc1 ; c1ccc(-c2ccc(-n3c4ccccc4c4c(-c5cncc6c7ccccc7n(-c7ccccc7)c56)cccc43)cc2)cc1 ; c1ccc(-c2nc(-c3ccccc3)nc(-n3c4ccccc4c4c(-c5cncc6c7ccccc7n(-c7ccccc7)c56)cccc43)n2)cc1]

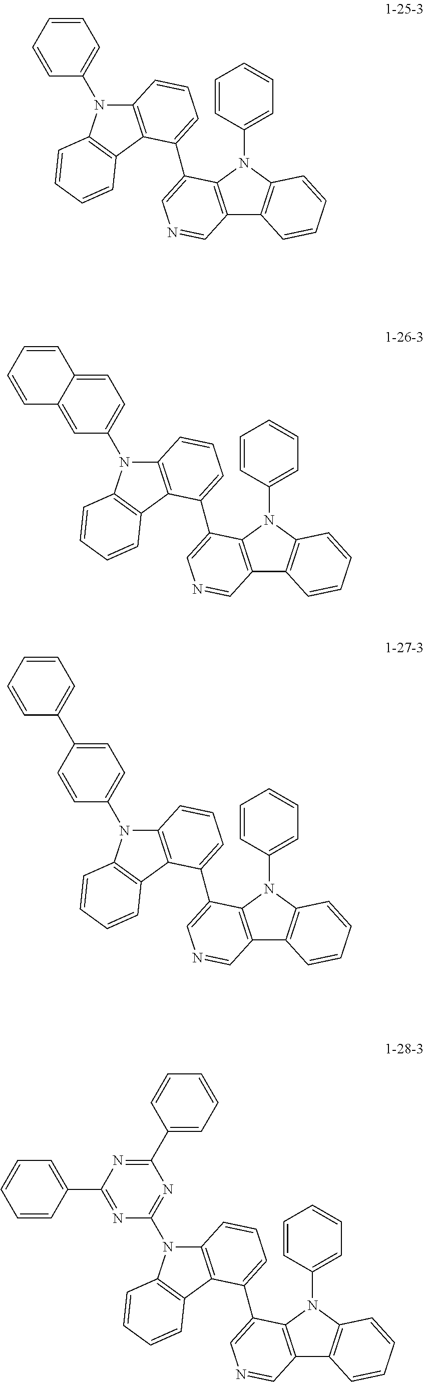 [SMILES: c1ccc(-n2c3ccccc3c3c(-c4cncc5c6ccccc6n(-c6ccccc6)c45)cccc32)cc1 ; c1ccc(-n2c3ccccc3c3cncc(-c4cccc5c4c4ccccc4n5-c4ccc5ccccc5c4)c32)cc1 ; c1ccc(-c2ccc(-n3c4ccccc4c4c(-c5cncc6c7ccccc7n(-c7ccccc7)c56)cccc43)cc2)cc1 ; c1ccc(-c2nc(-c3ccccc3)nc(-n3c4ccccc4c4c(-c5cncc6c7ccccc7n(-c7ccccc7)c56)cccc43)n2)cc1]

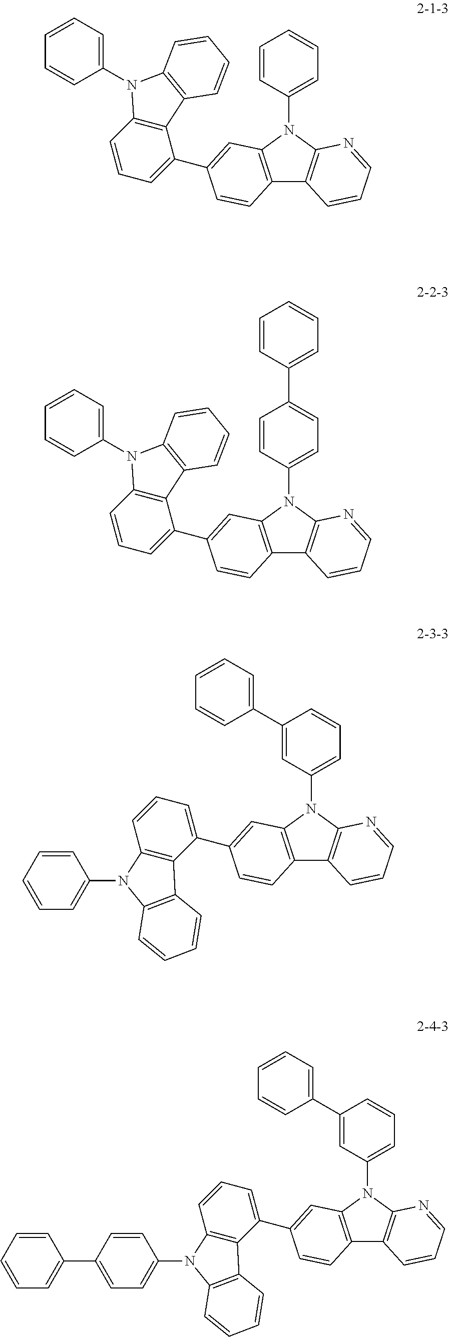 [SMILES: c1ccc(-n2c3ccccc3c3c(-c4ccc5c6cccnc6n(-c6ccccc6)c5c4)cccc32)cc1 ; c1ccc(-c2ccc(-n3c4cc(-c5cccc6c5c5ccccc5n6-c5ccccc5)ccc4c4cccnc43)cc2)cc1 ; c1ccc(-c2cccc(-n3c4cc(-c5cccc6c5c5ccccc5n6-c5ccccc5)ccc4c4cccnc43)c2)cc1 ; c1ccc(-c2ccc(-n3c4ccccc4c4c(-c5ccc6c7cccnc7n(-c7cccc(-c8ccccc8)c7)c6c5)cccc43)cc2)cc1]

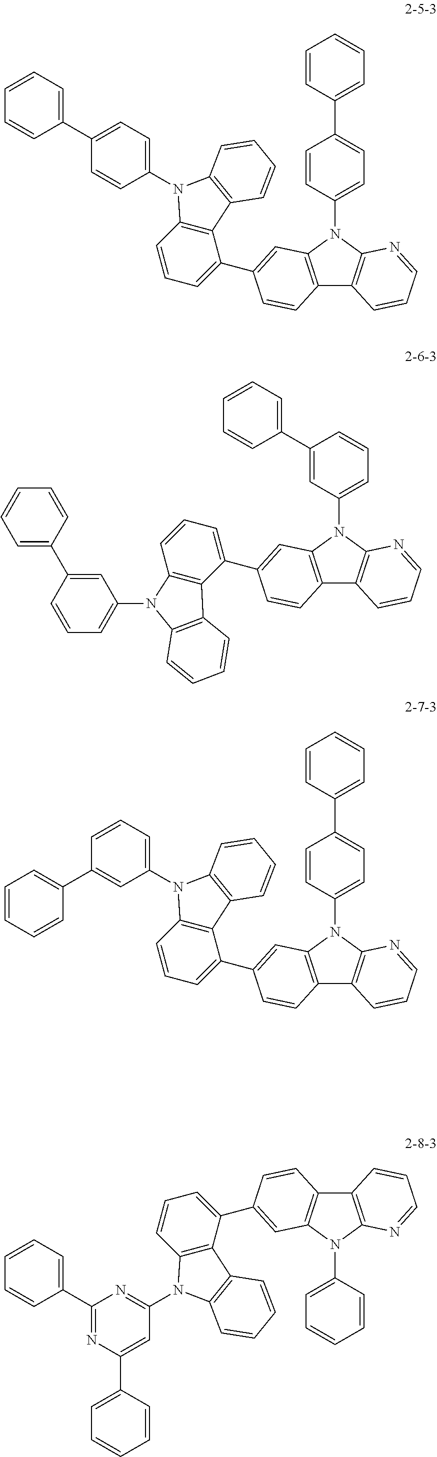 [SMILES: c1ccc(-c2ccc(-n3c4ccccc4c4c(-c5ccc6c7cccnc7n(-c7ccc(-c8ccccc8)cc7)c6c5)cccc43)cc2)cc1 ; c1ccc(-c2cccc(-n3c4ccccc4c4c(-c5ccc6c7cccnc7n(-c7cccc(-c8ccccc8)c7)c6c5)cccc43)c2)cc1 ; c1ccc(-c2ccc(-n3c4cc(-c5cccc6c5c5ccccc5n6-c5cccc(-c6ccccc6)c5)ccc4c4cccnc43)cc2)cc1 ; c1ccc(-c2cc(-n3c4ccccc4c4c(-c5ccc6c7cccnc7n(-c7ccccc7)c6c5)cccc43)nc(-c3ccccc3)n2)cc1]

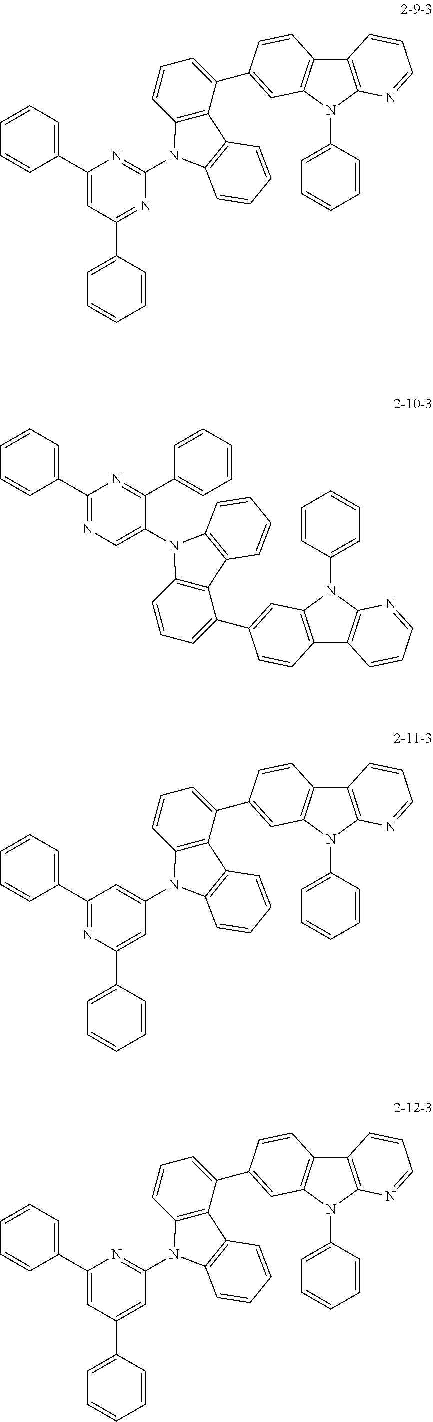 [SMILES: c1ccc(-c2cc(-c3ccccc3)nc(-n3c4ccccc4c4c(-c5ccc6c7cccnc7n(-c7ccccc7)c6c5)cccc43)n2)cc1 ; c1ccc(-c2ncc(-n3c4ccccc4c4c(-c5ccc6c7cccnc7n(-c7ccccc7)c6c5)cccc43)c(-c3ccccc3)n2)cc1 ; c1ccc(-c2cc(-n3c4ccccc4c4c(-c5ccc6c7cccnc7n(-c7ccccc7)c6c5)cccc43)cc(-c3ccccc3)n2)cc1 ; c1ccc(-c2cc(-c3ccccc3)nc(-n3c4ccccc4c4c(-c5ccc6c7cccnc7n(-c7ccccc7)c6c5)cccc43)c2)cc1]

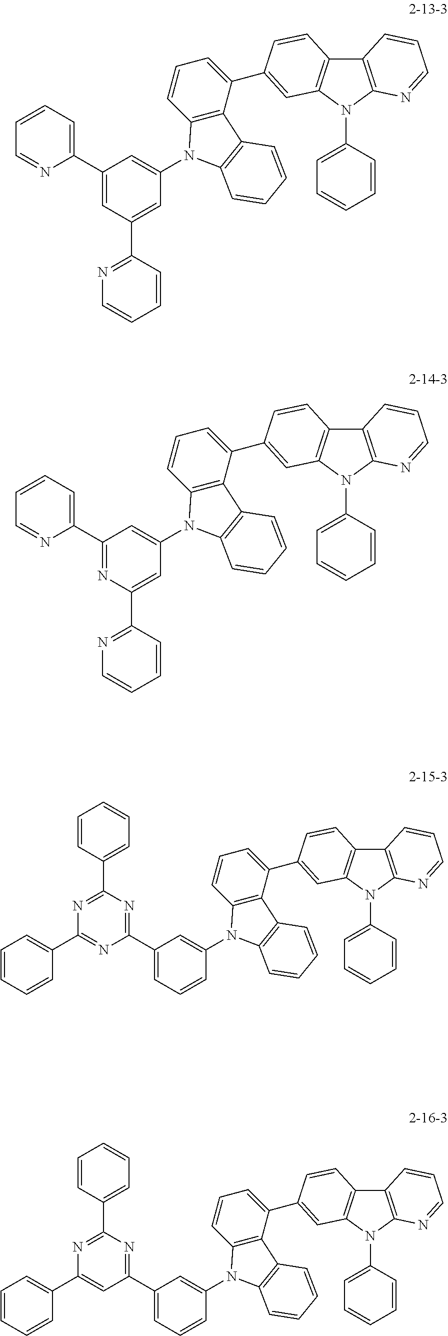 [SMILES: c1ccc(-n2c3cc(-c4cccc5c4c4ccccc4n5-c4cc(-c5ccccn5)cc(-c5ccccn5)c4)ccc3c3cccnc32)cc1 ; c1ccc(-n2c3cc(-c4cccc5c4c4ccccc4n5-c4cc(-c5ccccn5)nc(-c5ccccn5)c4)ccc3c3cccnc32)cc1 ; c1ccc(-c2nc(-c3ccccc3)nc(-c3cccc(-n4c5ccccc5c5c(-c6ccc7c8cccnc8n(-c8ccccc8)c7c6)cccc54)c3)n2)cc1 ; c1ccc(-c2cc(-c3cccc(-n4c5ccccc5c5c(-c6ccc7c8cccnc8n(-c8ccccc8)c7c6)cccc54)c3)nc(-c3ccccc3)n2)cc1]

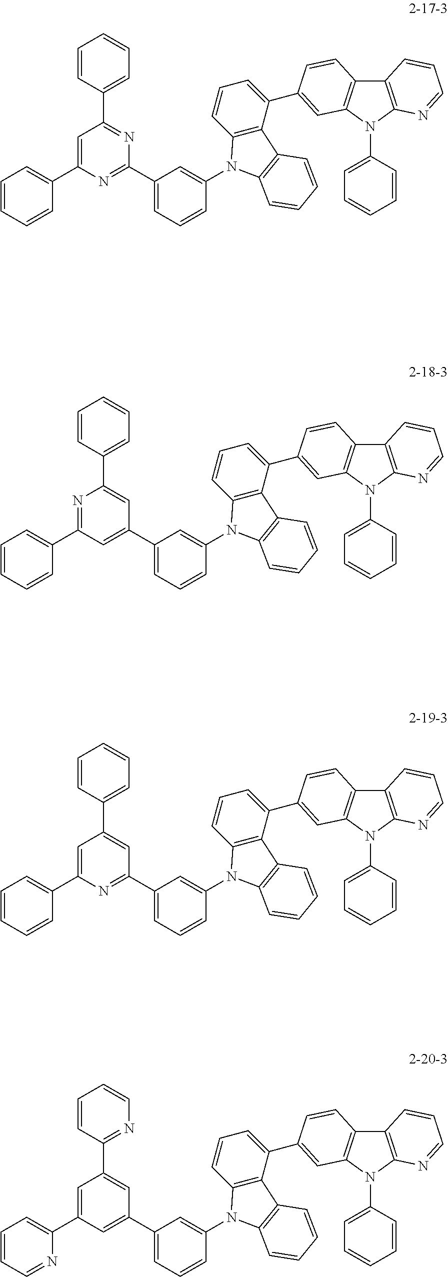 [SMILES: c1ccc(-c2cc(-c3ccccc3)nc(-c3cccc(-n4c5ccccc5c5c(-c6ccc7c8cccnc8n(-c8ccccc8)c7c6)cccc54)c3)n2)cc1 ; c1ccc(-c2cc(-c3cccc(-n4c5ccccc5c5c(-c6ccc7c8cccnc8n(-c8ccccc8)c7c6)cccc54)c3)cc(-c3ccccc3)n2)cc1 ; c1ccc(-c2cc(-c3ccccc3)nc(-c3cccc(-n4c5ccccc5c5c(-c6ccc7c8cccnc8n(-c8ccccc8)c7c6)cccc54)c3)c2)cc1 ; c1ccc(-n2c3cc(-c4cccc5c4c4ccccc4n5-c4cccc(-c5cc(-c6ccccn6)cc(-c6ccccn6)c5)c4)ccc3c3cccnc32)cc1]

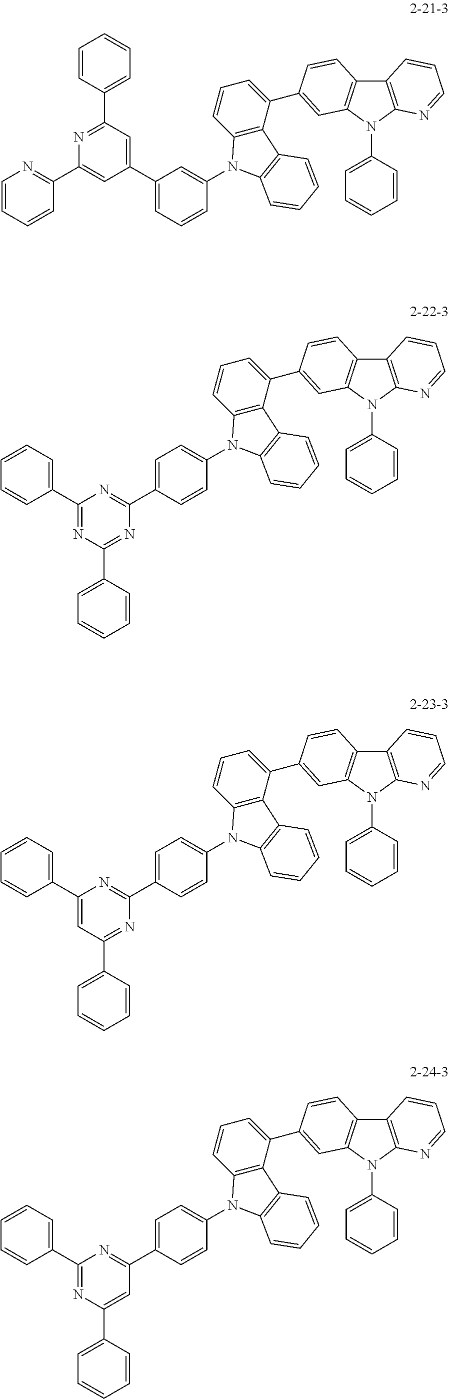 [SMILES: c1ccc(-c2cc(-c3cccc(-n4c5ccccc5c5c(-c6ccc7c8cccnc8n(-c8ccccc8)c7c6)cccc54)c3)cc(-c3ccccn3)n2)cc1 ; c1ccc(-c2nc(-c3ccccc3)nc(-c3ccc(-n4c5ccccc5c5c(-c6ccc7c8cccnc8n(-c8ccccc8)c7c6)cccc54)cc3)n2)cc1 ; c1ccc(-c2cc(-c3ccccc3)nc(-c3ccc(-n4c5ccccc5c5c(-c6ccc7c8cccnc8n(-c8ccccc8)c7c6)cccc54)cc3)n2)cc1 ; c1ccc(-c2cc(-c3ccc(-n4c5ccccc5c5c(-c6ccc7c8cccnc8n(-c8ccccc8)c7c6)cccc54)cc3)nc(-c3ccccc3)n2)cc1]

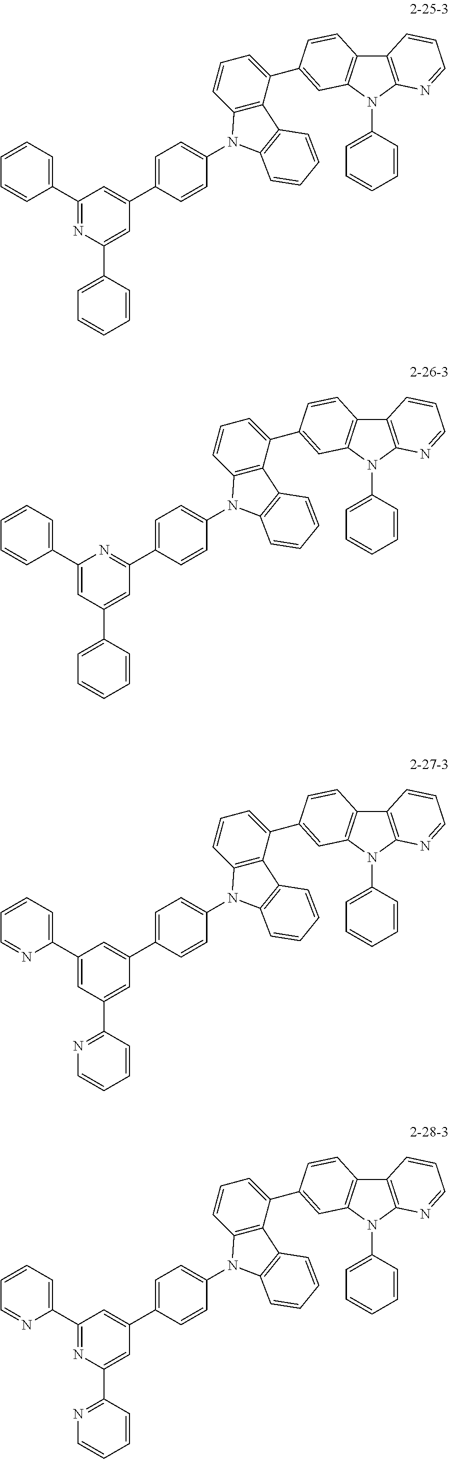 [SMILES: c1ccc(-c2cc(-c3ccc(-n4c5ccccc5c5c(-c6ccc7c8cccnc8n(-c8ccccc8)c7c6)cccc54)cc3)cc(-c3ccccc3)n2)cc1 ; c1ccc(-c2cc(-c3ccccc3)nc(-c3ccc(-n4c5ccccc5c5c(-c6ccc7c8cccnc8n(-c8ccccc8)c7c6)cccc54)cc3)c2)cc1 ; c1ccc(-n2c3cc(-c4cccc5c4c4ccccc4n5-c4ccc(-c5cc(-c6ccccn6)cc(-c6ccccn6)c5)cc4)ccc3c3cccnc32)cc1 ; c1ccc(-n2c3cc(-c4cccc5c4c4ccccc4n5-c4ccc(-c5cc(-c6ccccn6)nc(-c6ccccn6)c5)cc4)ccc3c3cccnc32)cc1]

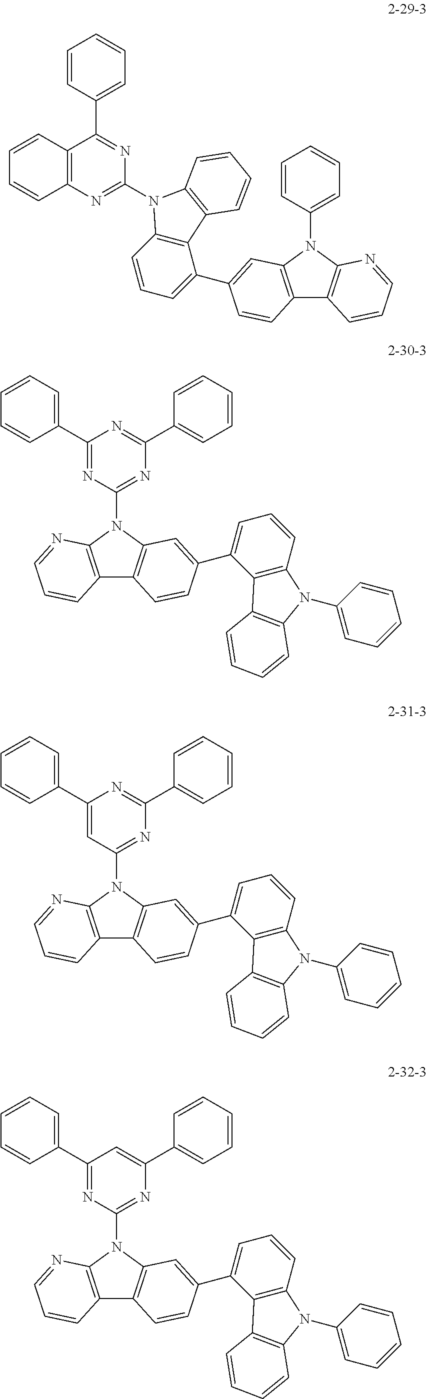 [SMILES: c1ccc(-c2nc(-n3c4ccccc4c4c(-c5ccc6c7cccnc7n(-c7ccccc7)c6c5)cccc43)nc3ccccc23)cc1 ; c1ccc(-c2nc(-c3ccccc3)nc(-n3c4cc(-c5cccc6c5c5ccccc5n6-c5ccccc5)ccc4c4cccnc43)n2)cc1 ; c1ccc(-c2cc(-n3c4cc(-c5cccc6c5c5ccccc5n6-c5ccccc5)ccc4c4cccnc43)nc(-c3ccccc3)n2)cc1 ; c1ccc(-c2cc(-c3ccccc3)nc(-n3c4cc(-c5cccc6c5c5ccccc5n6-c5ccccc5)ccc4c4cccnc43)n2)cc1]

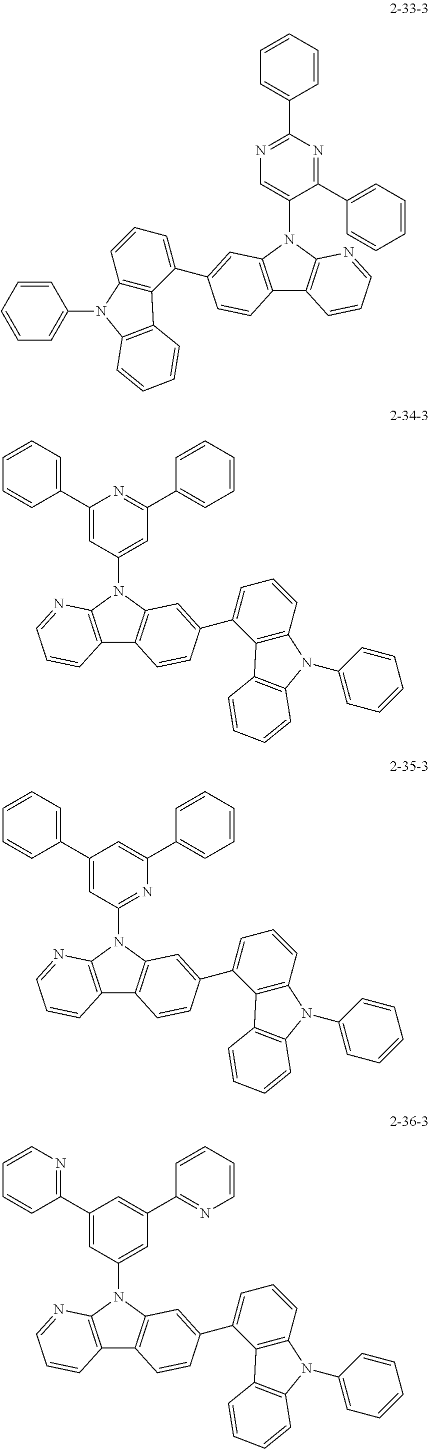 [SMILES: c1ccc(-c2ncc(-n3c4cc(-c5cccc6c5c5ccccc5n6-c5ccccc5)ccc4c4cccnc43)c(-c3ccccc3)n2)cc1 ; c1ccc(-c2cc(-n3c4cc(-c5cccc6c5c5ccccc5n6-c5ccccc5)ccc4c4cccnc43)cc(-c3ccccc3)n2)cc1 ; c1ccc(-c2cc(-c3ccccc3)nc(-n3c4cc(-c5cccc6c5c5ccccc5n6-c5ccccc5)ccc4c4cccnc43)c2)cc1 ; c1ccc(-n2c3ccccc3c3c(-c4ccc5c6cccnc6n(-c6cc(-c7ccccn7)cc(-c7ccccn7)c6)c5c4)cccc32)cc1]

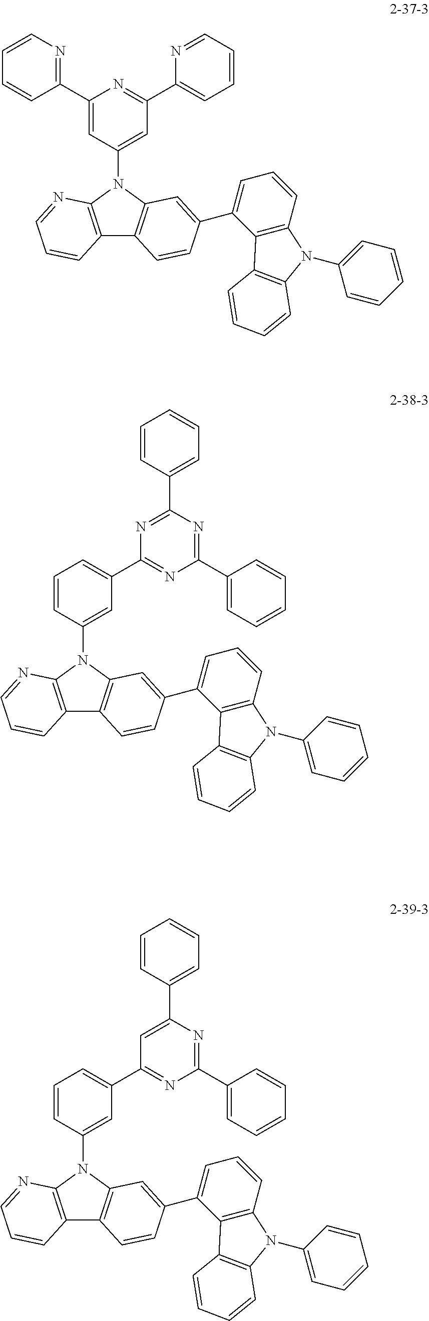 [SMILES: c1ccc(-n2c3ccccc3c3c(-c4ccc5c6cccnc6n(-c6cc(-c7ccccn7)nc(-c7ccccn7)c6)c5c4)cccc32)cc1 ; c1ccc(-c2nc(-c3ccccc3)nc(-c3cccc(-n4c5cc(-c6cccc7c6c6ccccc6n7-c6ccccc6)ccc5c5cccnc54)c3)n2)cc1 ; c1ccc(-c2cc(-c3cccc(-n4c5cc(-c6cccc7c6c6ccccc6n7-c6ccccc6)ccc5c5cccnc54)c3)nc(-c3ccccc3)n2)cc1]

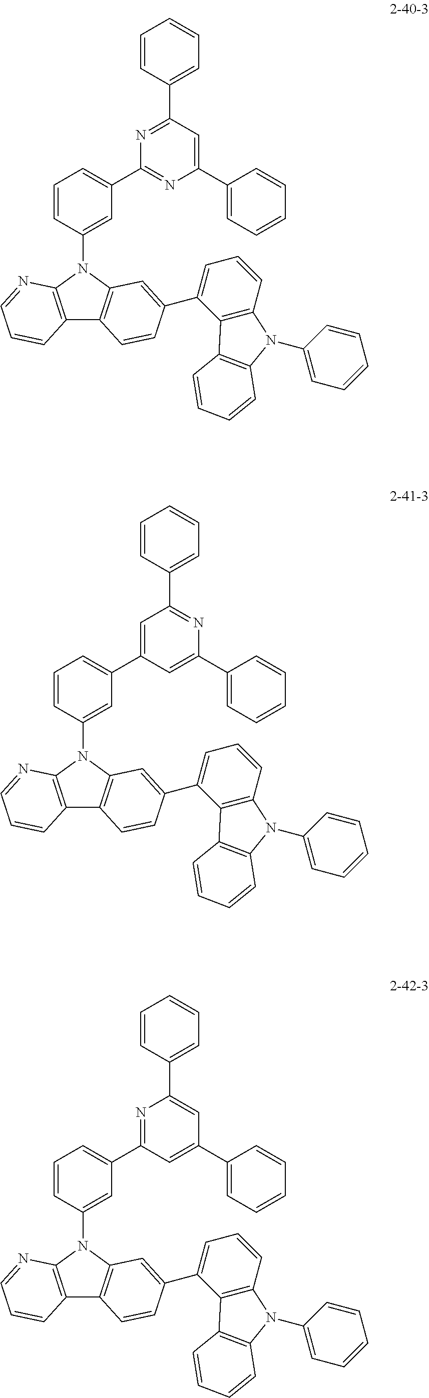 [SMILES: c1ccc(-c2cc(-c3ccccc3)nc(-c3cccc(-n4c5cc(-c6cccc7c6c6ccccc6n7-c6ccccc6)ccc5c5cccnc54)c3)n2)cc1 ; c1ccc(-c2cc(-c3cccc(-n4c5cc(-c6cccc7c6c6ccccc6n7-c6ccccc6)ccc5c5cccnc54)c3)cc(-c3ccccc3)n2)cc1 ; c1ccc(-c2cc(-c3ccccc3)nc(-c3cccc(-n4c5cc(-c6cccc7c6c6ccccc6n7-c6ccccc6)ccc5c5cccnc54)c3)c2)cc1]

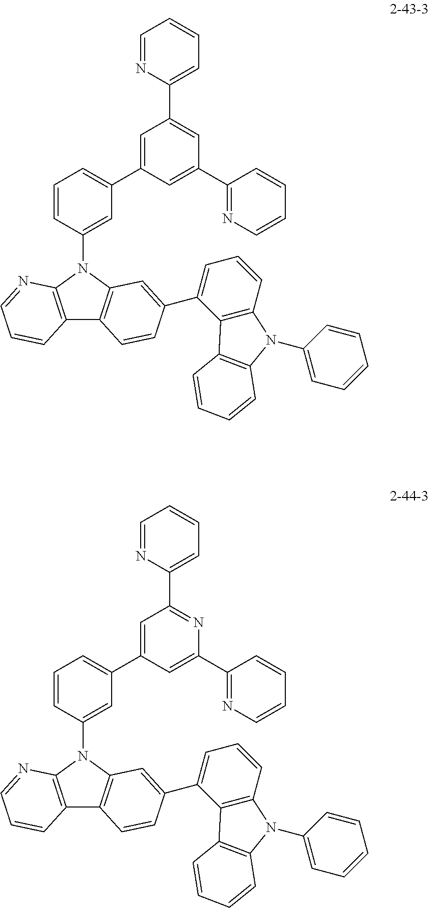 [SMILES: c1ccc(-n2c3ccccc3c3c(-c4ccc5c6cccnc6n(-c6cccc(-c7cc(-c8ccccn8)cc(-c8ccccn8)c7)c6)c5c4)cccc32)cc1 ; c1ccc(-n2c3ccccc3c3c(-c4ccc5c6cccnc6n(-c6cccc(-c7cc(-c8ccccn8)nc(-c8ccccn8)c7)c6)c5c4)cccc32)cc1]

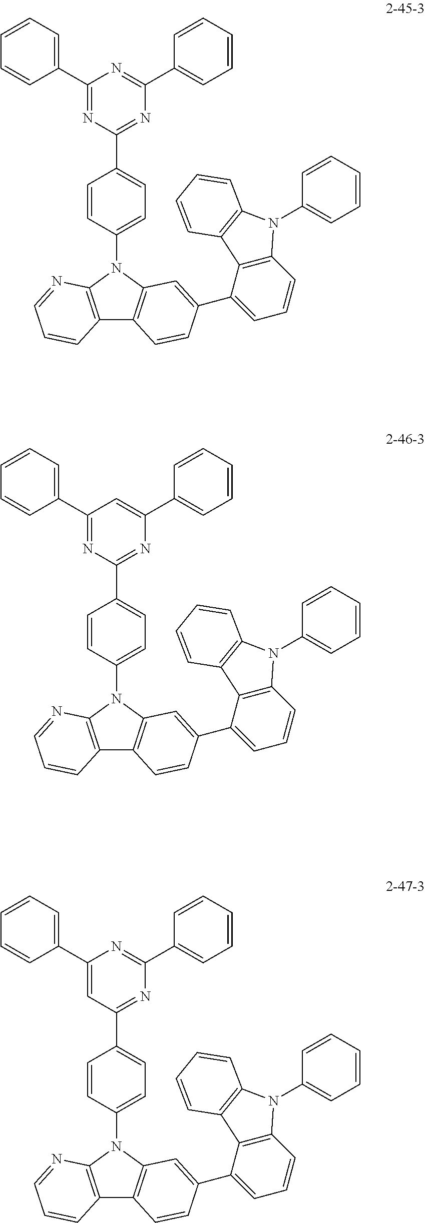 [SMILES: c1ccc(-c2nc(-c3ccccc3)nc(-c3ccc(-n4c5cc(-c6cccc7c6c6ccccc6n7-c6ccccc6)ccc5c5cccnc54)cc3)n2)cc1 ; c1ccc(-c2cc(-c3ccccc3)nc(-c3ccc(-n4c5cc(-c6cccc7c6c6ccccc6n7-c6ccccc6)ccc5c5cccnc54)cc3)n2)cc1 ; c1ccc(-c2cc(-c3ccc(-n4c5cc(-c6cccc7c6c6ccccc6n7-c6ccccc6)ccc5c5cccnc54)cc3)nc(-c3ccccc3)n2)cc1]

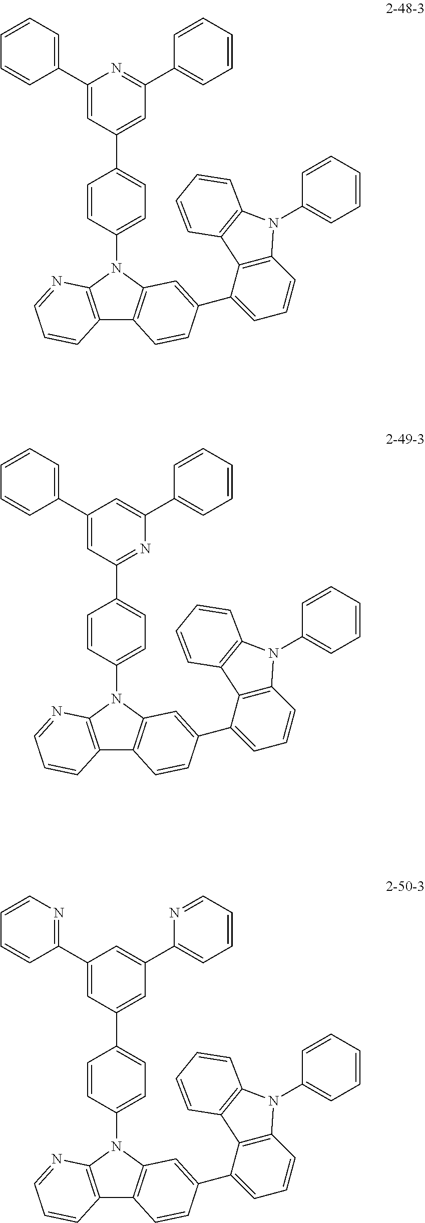 [SMILES: c1ccc(-c2cc(-c3ccc(-n4c5cc(-c6cccc7c6c6ccccc6n7-c6ccccc6)ccc5c5cccnc54)cc3)cc(-c3ccccc3)n2)cc1 ; c1ccc(-c2cc(-c3ccccc3)nc(-c3ccc(-n4c5cc(-c6cccc7c6c6ccccc6n7-c6ccccc6)ccc5c5cccnc54)cc3)c2)cc1 ; c1ccc(-n2c3ccccc3c3c(-c4ccc5c6cccnc6n(-c6ccc(-c7cc(-c8ccccn8)cc(-c8ccccn8)c7)cc6)c5c4)cccc32)cc1]

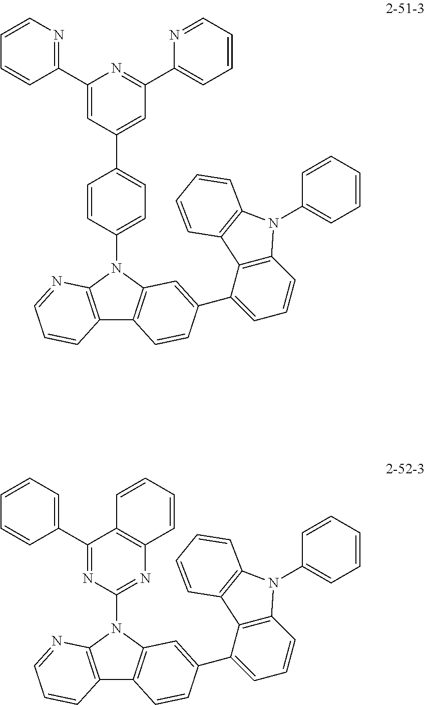 [SMILES: c1ccc(-n2c3ccccc3c3c(-c4ccc5c6cccnc6n(-c6ccc(-c7cc(-c8ccccn8)nc(-c8ccccn8)c7)cc6)c5c4)cccc32)cc1 ; c1ccc(-c2nc(-n3c4cc(-c5cccc6c5c5ccccc5n6-c5ccccc5)ccc4c4cccnc43)nc3ccccc23)cc1]

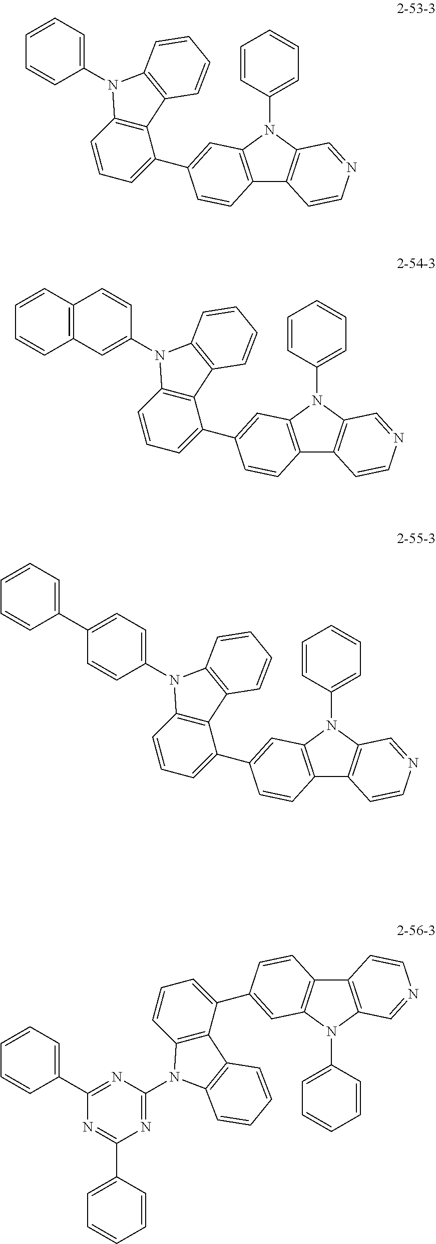 [SMILES: c1ccc(-n2c3cnccc3c3ccc(-c4cccc5c4c4ccccc4n5-c4ccccc4)cc32)cc1 ; c1ccc(-n2c3cnccc3c3ccc(-c4cccc5c4c4ccccc4n5-c4ccc5ccccc5c4)cc32)cc1 ; c1ccc(-c2ccc(-n3c4ccccc4c4c(-c5ccc6c7ccncc7n(-c7ccccc7)c6c5)cccc43)cc2)cc1 ; c1ccc(-c2nc(-c3ccccc3)nc(-n3c4ccccc4c4c(-c5ccc6c7ccncc7n(-c7ccccc7)c6c5)cccc43)n2)cc1]

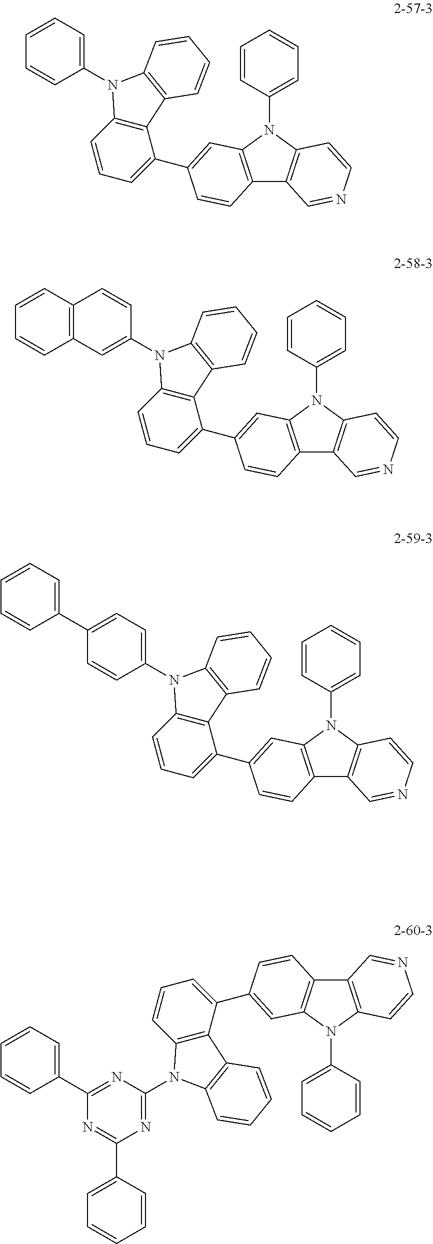 [SMILES: c1ccc(-n2c3ccncc3c3ccc(-c4cccc5c4c4ccccc4n5-c4ccccc4)cc32)cc1 ; c1ccc(-n2c3ccncc3c3ccc(-c4cccc5c4c4ccccc4n5-c4ccc5ccccc5c4)cc32)cc1 ; c1ccc(-c2ccc(-n3c4ccccc4c4c(-c5ccc6c7cnccc7n(-c7ccccc7)c6c5)cccc43)cc2)cc1 ; c1ccc(-c2nc(-c3ccccc3)nc(-n3c4ccccc4c4c(-c5ccc6c7cnccc7n(-c7ccccc7)c6c5)cccc43)n2)cc1]

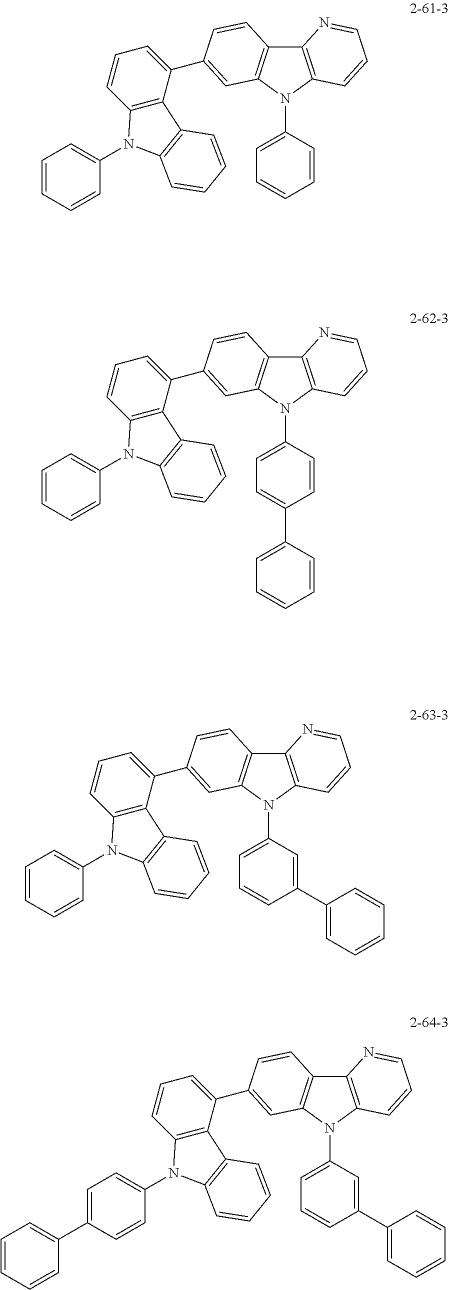 [SMILES: c1ccc(-n2c3cc(-c4cccc5c4c4ccccc4n5-c4ccccc4)ccc3c3ncccc32)cc1 ; c1ccc(-c2ccc(-n3c4cc(-c5cccc6c5c5ccccc5n6-c5ccccc5)ccc4c4ncccc43)cc2)cc1 ; c1ccc(-c2cccc(-n3c4cc(-c5cccc6c5c5ccccc5n6-c5ccccc5)ccc4c4ncccc43)c2)cc1 ; c1ccc(-c2ccc(-n3c4ccccc4c4c(-c5ccc6c7ncccc7n(-c7cccc(-c8ccccc8)c7)c6c5)cccc43)cc2)cc1]

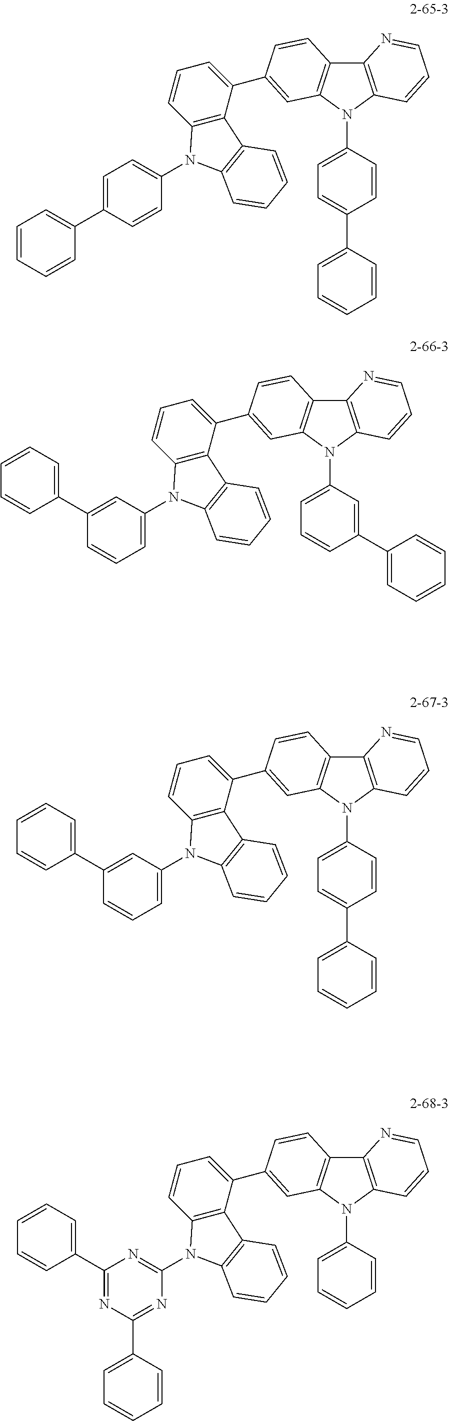 [SMILES: c1ccc(-c2ccc(-n3c4cc(-c5cccc6c5c5ccccc5n6-c5ccc(-c6ccccc6)cc5)ccc4c4ncccc43)cc2)cc1 ; c1ccc(-c2cccc(-n3c4cc(-c5cccc6c5c5ccccc5n6-c5cccc(-c6ccccc6)c5)ccc4c4ncccc43)c2)cc1 ; c1ccc(-c2ccc(-n3c4cc(-c5cccc6c5c5ccccc5n6-c5cccc(-c6ccccc6)c5)ccc4c4ncccc43)cc2)cc1 ; c1ccc(-c2nc(-c3ccccc3)nc(-n3c4ccccc4c4c(-c5ccc6c7ncccc7n(-c7ccccc7)c6c5)cccc43)n2)cc1]

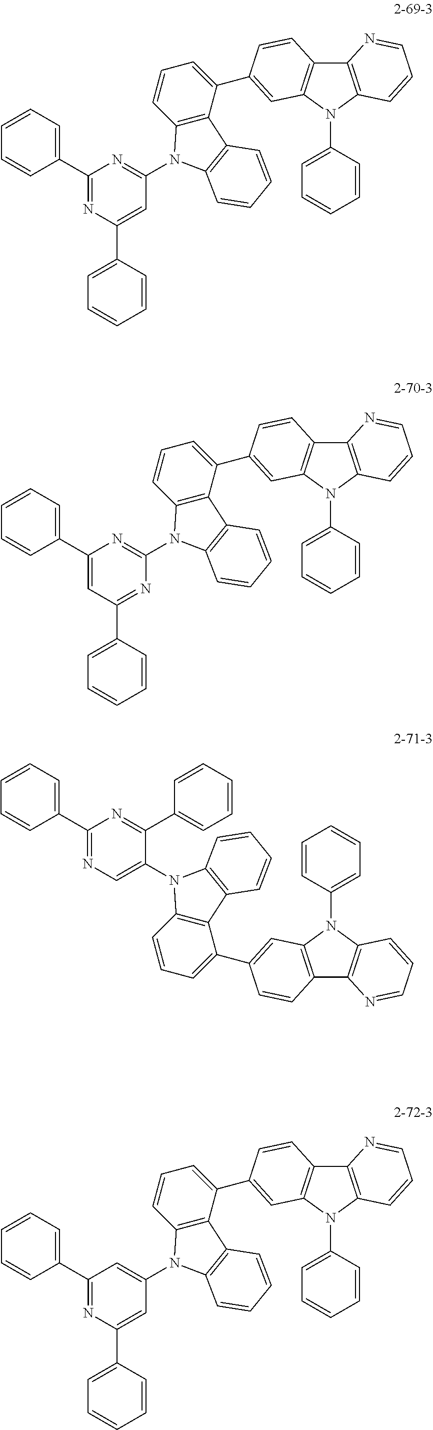 [SMILES: c1ccc(-c2cc(-n3c4ccccc4c4c(-c5ccc6c7ncccc7n(-c7ccccc7)c6c5)cccc43)nc(-c3ccccc3)n2)cc1 ; c1ccc(-c2cc(-c3ccccc3)nc(-n3c4ccccc4c4c(-c5ccc6c7ncccc7n(-c7ccccc7)c6c5)cccc43)n2)cc1 ; c1ccc(-c2ncc(-n3c4ccccc4c4c(-c5ccc6c7ncccc7n(-c7ccccc7)c6c5)cccc43)c(-c3ccccc3)n2)cc1 ; c1ccc(-c2cc(-n3c4ccccc4c4c(-c5ccc6c7ncccc7n(-c7ccccc7)c6c5)cccc43)cc(-c3ccccc3)n2)cc1]

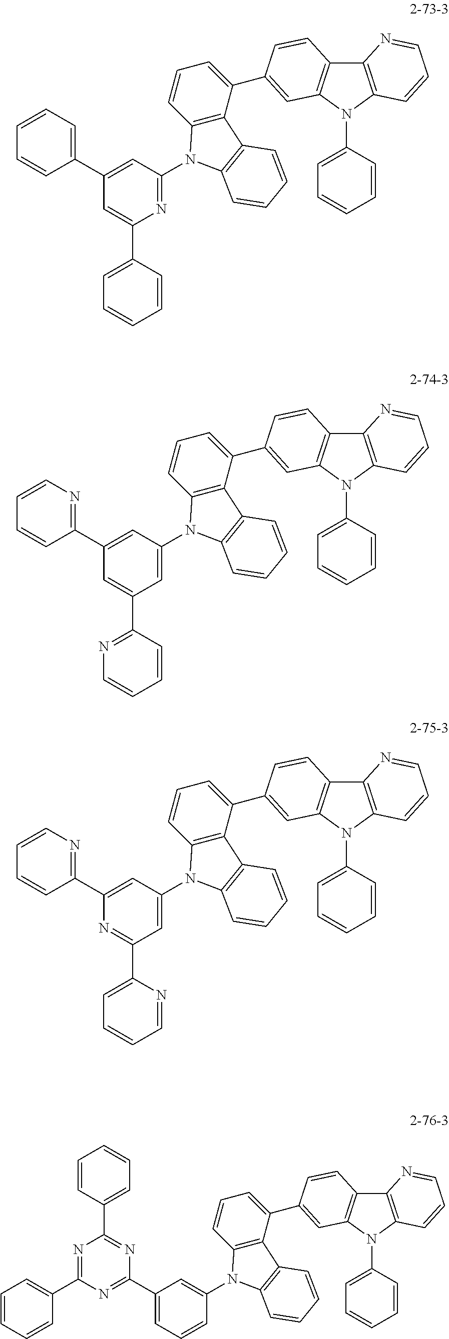 [SMILES: c1ccc(-c2cc(-c3ccccc3)nc(-n3c4ccccc4c4c(-c5ccc6c7ncccc7n(-c7ccccc7)c6c5)cccc43)c2)cc1 ; c1ccc(-n2c3cc(-c4cccc5c4c4ccccc4n5-c4cc(-c5ccccn5)cc(-c5ccccn5)c4)ccc3c3ncccc32)cc1 ; c1ccc(-n2c3cc(-c4cccc5c4c4ccccc4n5-c4cc(-c5ccccn5)nc(-c5ccccn5)c4)ccc3c3ncccc32)cc1 ; c1ccc(-c2nc(-c3ccccc3)nc(-c3cccc(-n4c5ccccc5c5c(-c6ccc7c8ncccc8n(-c8ccccc8)c7c6)cccc54)c3)n2)cc1]

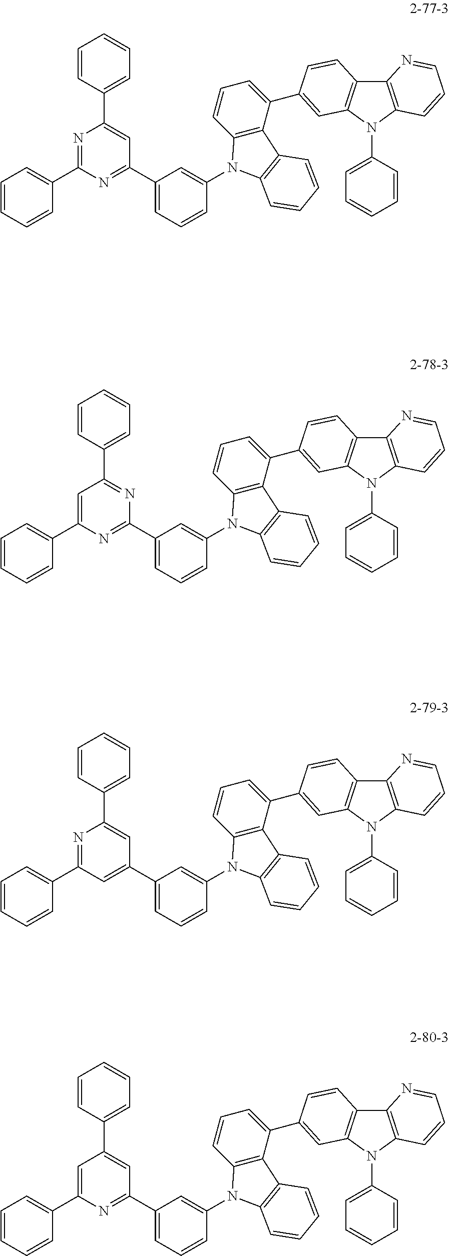 [SMILES: c1ccc(-c2cc(-c3cccc(-n4c5ccccc5c5c(-c6ccc7c8ncccc8n(-c8ccccc8)c7c6)cccc54)c3)nc(-c3ccccc3)n2)cc1 ; c1ccc(-c2cc(-c3ccccc3)nc(-c3cccc(-n4c5ccccc5c5c(-c6ccc7c8ncccc8n(-c8ccccc8)c7c6)cccc54)c3)n2)cc1 ; c1ccc(-c2cc(-c3cccc(-n4c5ccccc5c5c(-c6ccc7c8ncccc8n(-c8ccccc8)c7c6)cccc54)c3)cc(-c3ccccc3)n2)cc1 ; c1ccc(-c2cc(-c3ccccc3)nc(-c3cccc(-n4c5ccccc5c5c(-c6ccc7c8ncccc8n(-c8ccccc8)c7c6)cccc54)c3)c2)cc1]

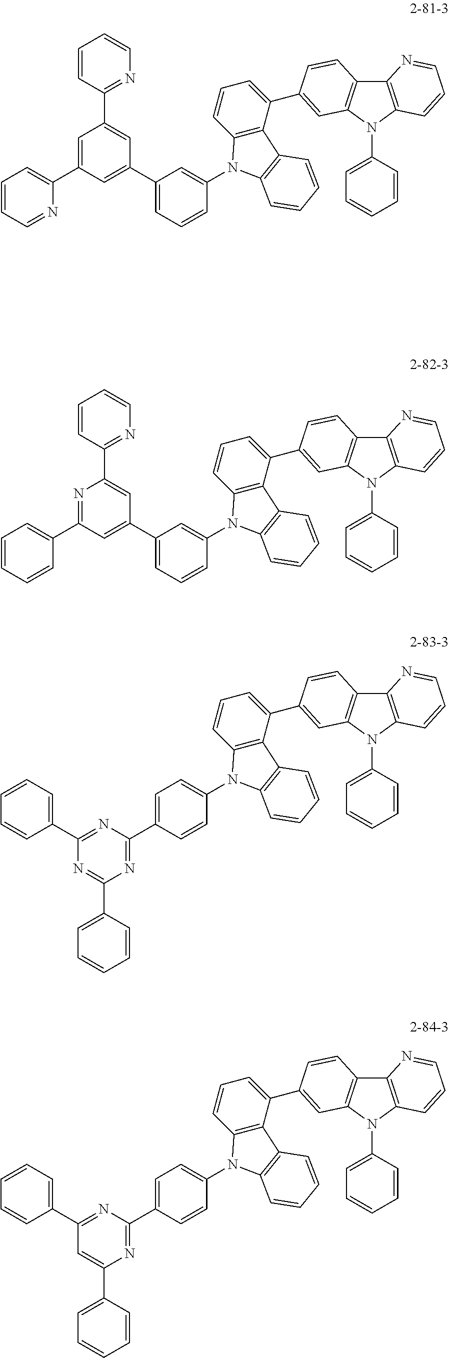 [SMILES: c1ccc(-n2c3cc(-c4cccc5c4c4ccccc4n5-c4cccc(-c5cc(-c6ccccn6)cc(-c6ccccn6)c5)c4)ccc3c3ncccc32)cc1 ; c1ccc(-c2cc(-c3cccc(-n4c5ccccc5c5c(-c6ccc7c8ncccc8n(-c8ccccc8)c7c6)cccc54)c3)cc(-c3ccccn3)n2)cc1 ; c1ccc(-c2nc(-c3ccccc3)nc(-c3ccc(-n4c5ccccc5c5c(-c6ccc7c8ncccc8n(-c8ccccc8)c7c6)cccc54)cc3)n2)cc1 ; c1ccc(-c2cc(-c3ccccc3)nc(-c3ccc(-n4c5ccccc5c5c(-c6ccc7c8ncccc8n(-c8ccccc8)c7c6)cccc54)cc3)n2)cc1]

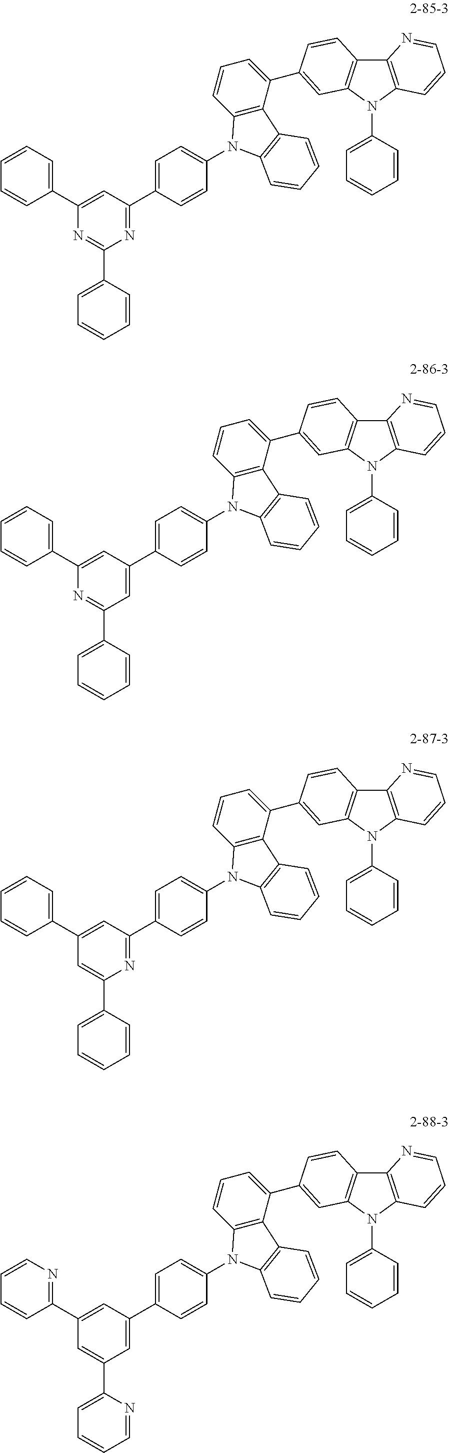 [SMILES: c1ccc(-c2cc(-c3ccc(-n4c5ccccc5c5c(-c6ccc7c8ncccc8n(-c8ccccc8)c7c6)cccc54)cc3)nc(-c3ccccc3)n2)cc1 ; c1ccc(-c2cc(-c3ccc(-n4c5ccccc5c5c(-c6ccc7c8ncccc8n(-c8ccccc8)c7c6)cccc54)cc3)cc(-c3ccccc3)n2)cc1 ; c1ccc(-c2cc(-c3ccccc3)nc(-c3ccc(-n4c5ccccc5c5c(-c6ccc7c8ncccc8n(-c8ccccc8)c7c6)cccc54)cc3)c2)cc1 ; c1ccc(-n2c3cc(-c4cccc5c4c4ccccc4n5-c4ccc(-c5cc(-c6ccccn6)cc(-c6ccccn6)c5)cc4)ccc3c3ncccc32)cc1]

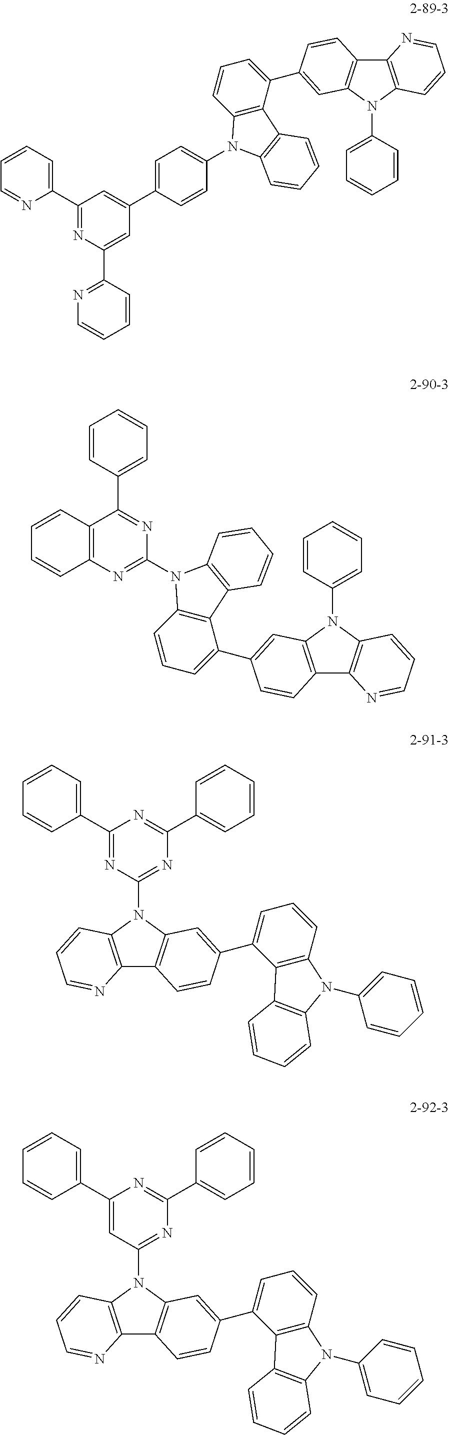 [SMILES: c1ccc(-n2c3cc(-c4cccc5c4c4ccccc4n5-c4ccc(-c5cc(-c6ccccn6)nc(-c6ccccn6)c5)cc4)ccc3c3ncccc32)cc1 ; c1ccc(-c2nc(-n3c4ccccc4c4c(-c5ccc6c7ncccc7n(-c7ccccc7)c6c5)cccc43)nc3ccccc23)cc1 ; c1ccc(-c2nc(-c3ccccc3)nc(-n3c4cc(-c5cccc6c5c5ccccc5n6-c5ccccc5)ccc4c4ncccc43)n2)cc1 ; c1ccc(-c2cc(-n3c4cc(-c5cccc6c5c5ccccc5n6-c5ccccc5)ccc4c4ncccc43)nc(-c3ccccc3)n2)cc1]

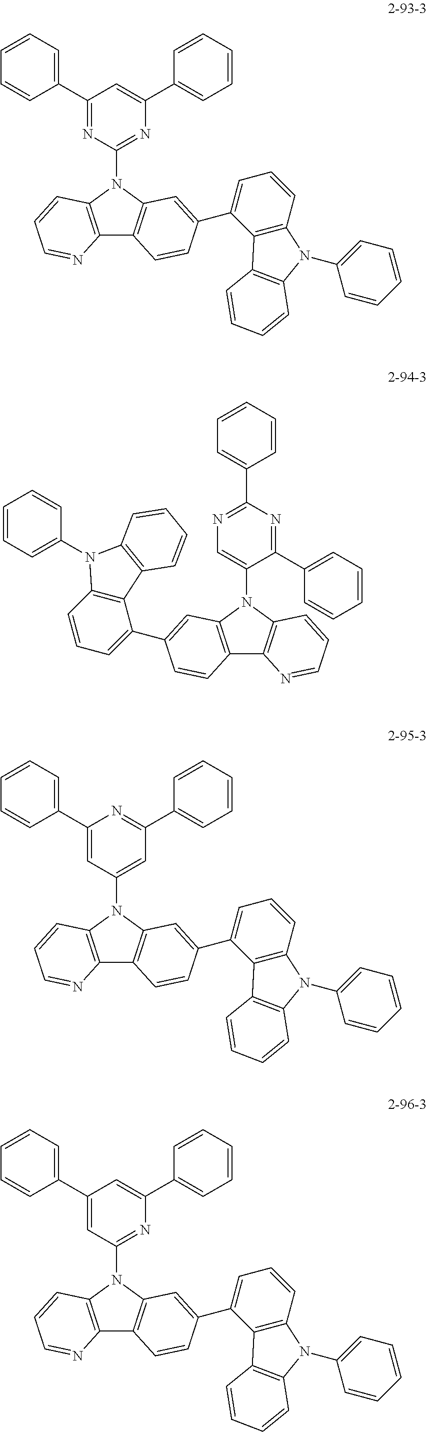 [SMILES: c1ccc(-c2cc(-c3ccccc3)nc(-n3c4cc(-c5cccc6c5c5ccccc5n6-c5ccccc5)ccc4c4ncccc43)n2)cc1 ; c1ccc(-c2ncc(-n3c4cc(-c5cccc6c5c5ccccc5n6-c5ccccc5)ccc4c4ncccc43)c(-c3ccccc3)n2)cc1 ; c1ccc(-c2cc(-n3c4cc(-c5cccc6c5c5ccccc5n6-c5ccccc5)ccc4c4ncccc43)cc(-c3ccccc3)n2)cc1 ; c1ccc(-c2cc(-c3ccccc3)nc(-n3c4cc(-c5cccc6c5c5ccccc5n6-c5ccccc5)ccc4c4ncccc43)c2)cc1]

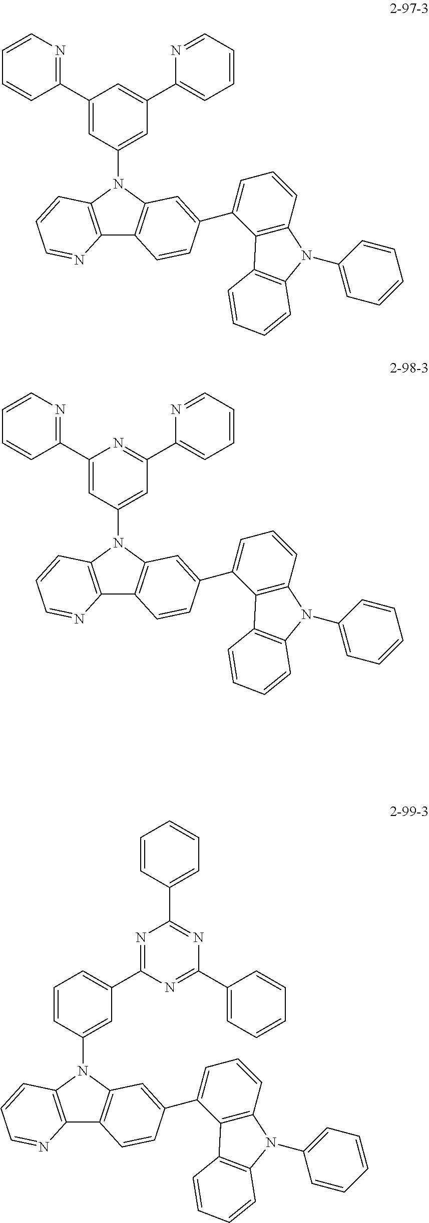 [SMILES: c1ccc(-n2c3ccccc3c3c(-c4ccc5c6ncccc6n(-c6cc(-c7ccccn7)cc(-c7ccccn7)c6)c5c4)cccc32)cc1 ; c1ccc(-n2c3ccccc3c3c(-c4ccc5c6ncccc6n(-c6cc(-c7ccccn7)nc(-c7ccccn7)c6)c5c4)cccc32)cc1 ; c1ccc(-c2nc(-c3ccccc3)nc(-c3cccc(-n4c5cc(-c6cccc7c6c6ccccc6n7-c6ccccc6)ccc5c5ncccc54)c3)n2)cc1]

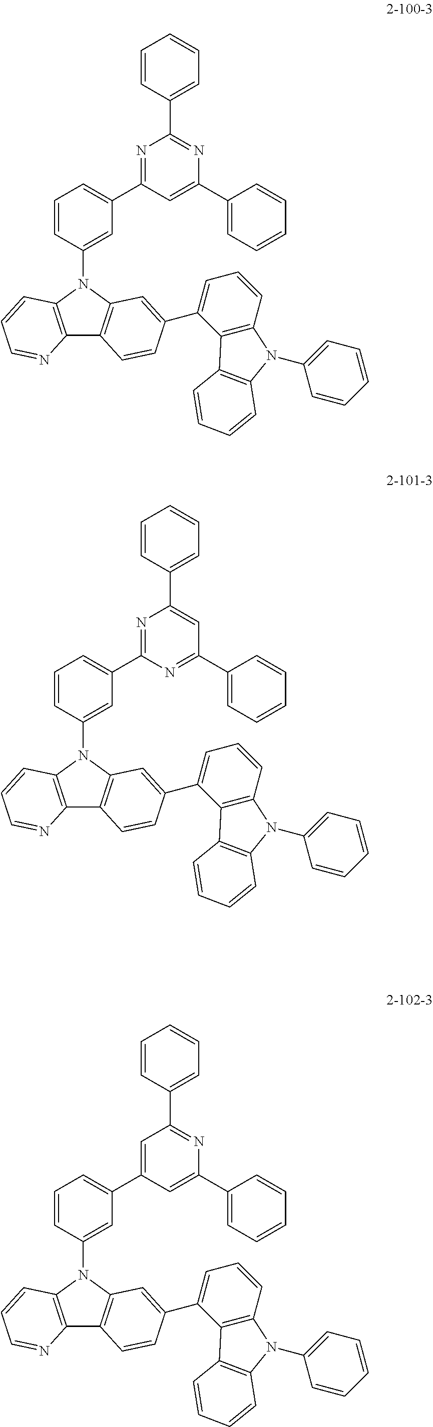 [SMILES: c1ccc(-c2cc(-c3cccc(-n4c5cc(-c6cccc7c6c6ccccc6n7-c6ccccc6)ccc5c5ncccc54)c3)nc(-c3ccccc3)n2)cc1 ; c1ccc(-c2cc(-c3ccccc3)nc(-c3cccc(-n4c5cc(-c6cccc7c6c6ccccc6n7-c6ccccc6)ccc5c5ncccc54)c3)n2)cc1 ; c1ccc(-c2cc(-c3cccc(-n4c5cc(-c6cccc7c6c6ccccc6n7-c6ccccc6)ccc5c5ncccc54)c3)cc(-c3ccccc3)n2)cc1]

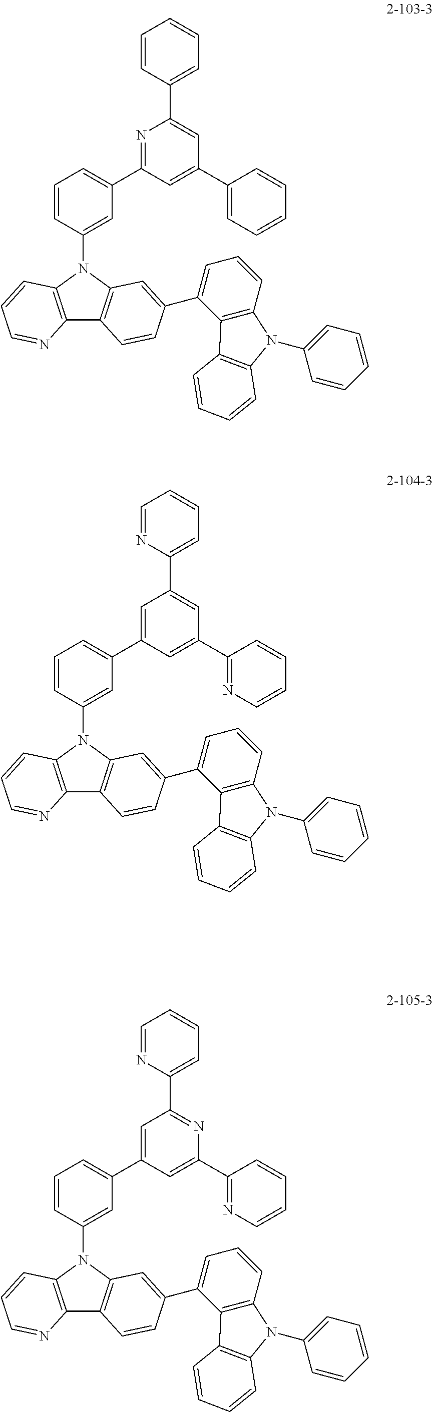 [SMILES: c1ccc(-c2cc(-c3ccccc3)nc(-c3cccc(-n4c5cc(-c6cccc7c6c6ccccc6n7-c6ccccc6)ccc5c5ncccc54)c3)c2)cc1 ; c1ccc(-n2c3ccccc3c3c(-c4ccc5c6ncccc6n(-c6cccc(-c7cc(-c8ccccn8)cc(-c8ccccn8)c7)c6)c5c4)cccc32)cc1 ; c1ccc(-n2c3ccccc3c3c(-c4ccc5c6ncccc6n(-c6cccc(-c7cc(-c8ccccn8)nc(-c8ccccn8)c7)c6)c5c4)cccc32)cc1]

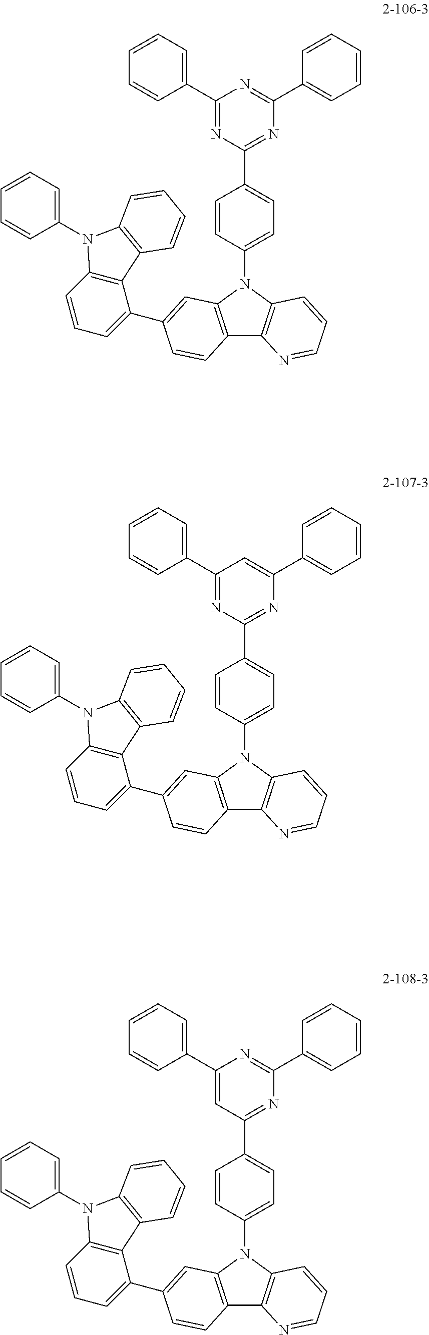 [SMILES: c1ccc(-c2nc(-c3ccccc3)nc(-c3ccc(-n4c5cc(-c6cccc7c6c6ccccc6n7-c6ccccc6)ccc5c5ncccc54)cc3)n2)cc1 ; c1ccc(-c2cc(-c3ccccc3)nc(-c3ccc(-n4c5cc(-c6cccc7c6c6ccccc6n7-c6ccccc6)ccc5c5ncccc54)cc3)n2)cc1 ; c1ccc(-c2cc(-c3ccc(-n4c5cc(-c6cccc7c6c6ccccc6n7-c6ccccc6)ccc5c5ncccc54)cc3)nc(-c3ccccc3)n2)cc1]

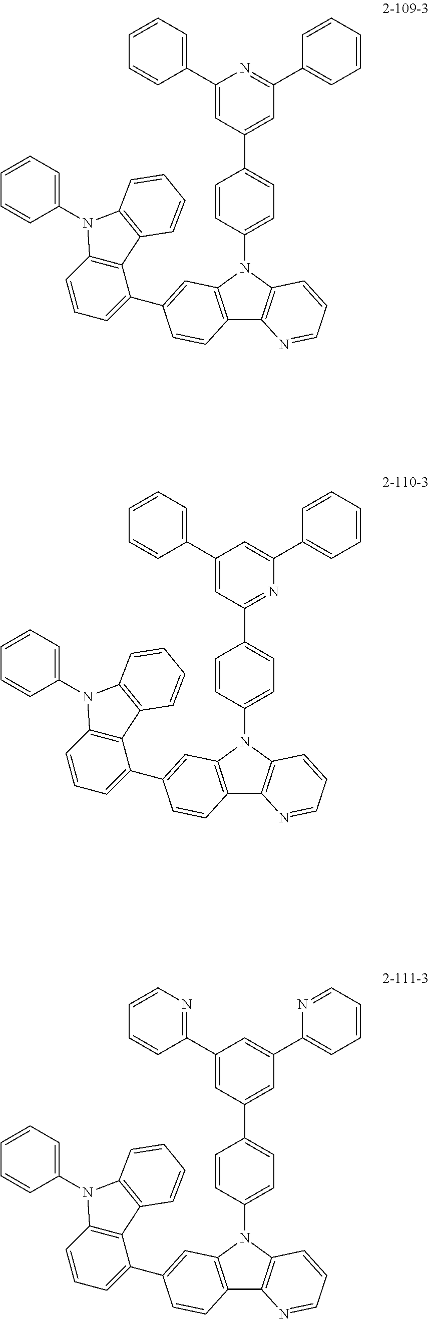 [SMILES: c1ccc(-c2cc(-c3ccc(-n4c5cc(-c6cccc7c6c6ccccc6n7-c6ccccc6)ccc5c5ncccc54)cc3)cc(-c3ccccc3)n2)cc1 ; c1ccc(-c2cc(-c3ccccc3)nc(-c3ccc(-n4c5cc(-c6cccc7c6c6ccccc6n7-c6ccccc6)ccc5c5ncccc54)cc3)c2)cc1 ; c1ccc(-n2c3ccccc3c3c(-c4ccc5c6ncccc6n(-c6ccc(-c7cc(-c8ccccn8)cc(-c8ccccn8)c7)cc6)c5c4)cccc32)cc1]

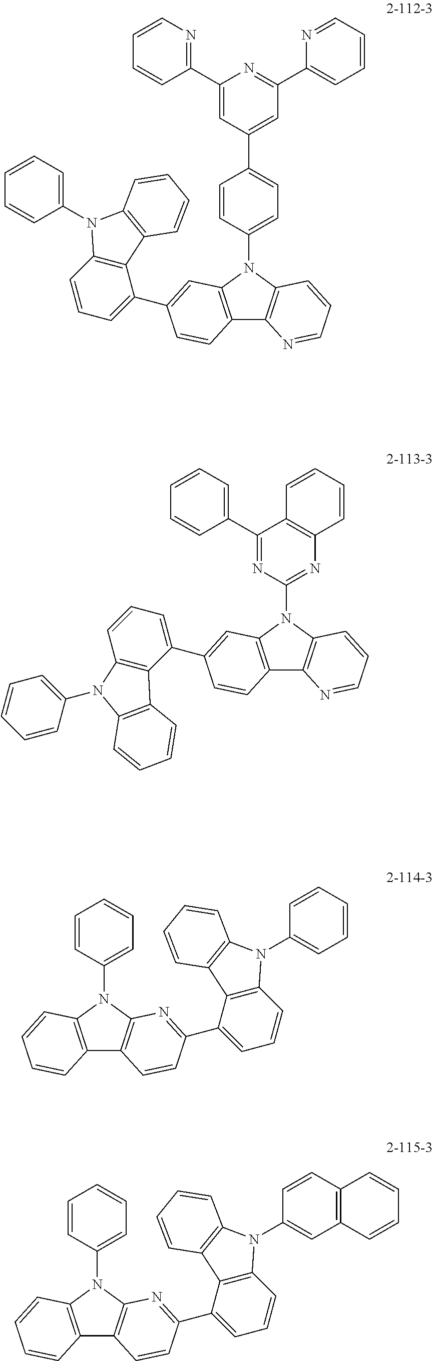 [SMILES: c1ccc(-n2c3ccccc3c3c(-c4ccc5c6ncccc6n(-c6ccc(-c7cc(-c8ccccn8)nc(-c8ccccn8)c7)cc6)c5c4)cccc32)cc1 ; c1ccc(-c2nc(-n3c4cc(-c5cccc6c5c5ccccc5n6-c5ccccc5)ccc4c4ncccc43)nc3ccccc23)cc1 ; c1ccc(-n2c3ccccc3c3c(-c4ccc5c6ccccc6n(-c6ccccc6)c5n4)cccc32)cc1 ; c1ccc(-n2c3ccccc3c3ccc(-c4cccc5c4c4ccccc4n5-c4ccc5ccccc5c4)nc32)cc1]

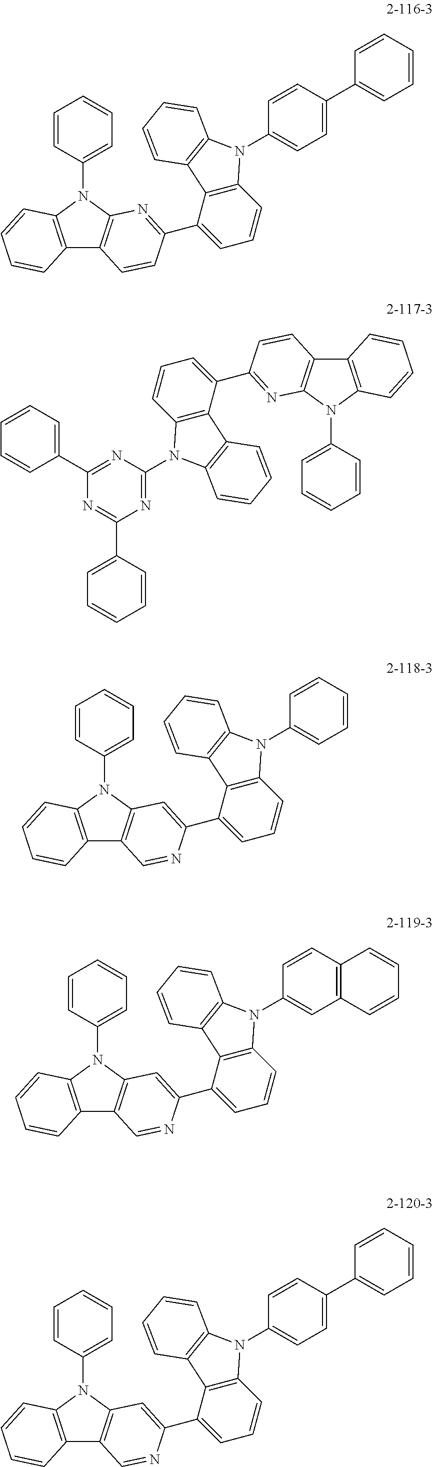 [SMILES: c1ccc(-c2ccc(-n3c4ccccc4c4c(-c5ccc6c7ccccc7n(-c7ccccc7)c6n5)cccc43)cc2)cc1 ; c1ccc(-c2nc(-c3ccccc3)nc(-n3c4ccccc4c4c(-c5ccc6c7ccccc7n(-c7ccccc7)c6n5)cccc43)n2)cc1 ; c1ccc(-n2c3ccccc3c3cnc(-c4cccc5c4c4ccccc4n5-c4ccccc4)cc32)cc1 ; c1ccc(-n2c3ccccc3c3cnc(-c4cccc5c4c4ccccc4n5-c4ccc5ccccc5c4)cc32)cc1 ; c1ccc(-c2ccc(-n3c4ccccc4c4c(-c5cc6c(cn5)c5ccccc5n6-c5ccccc5)cccc43)cc2)cc1]

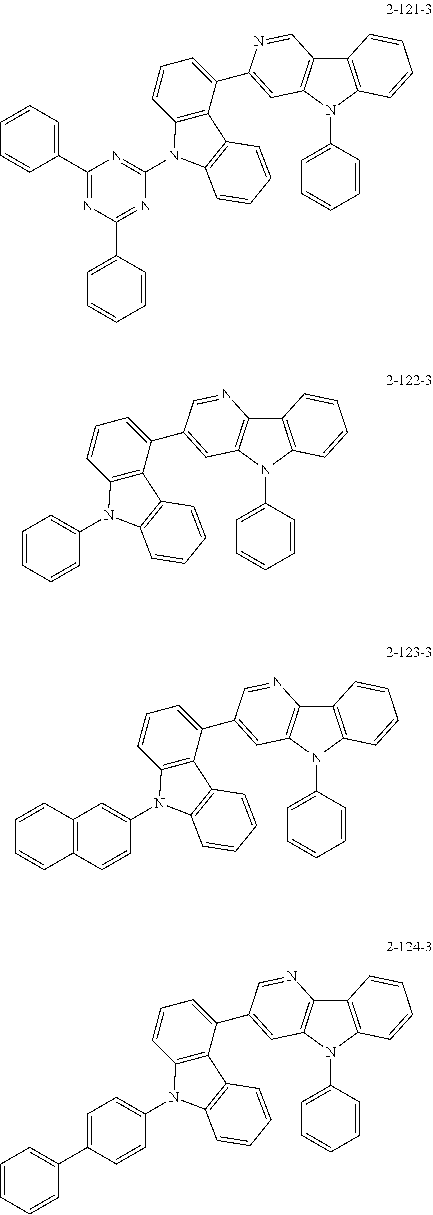 [SMILES: c1ccc(-c2nc(-c3ccccc3)nc(-n3c4ccccc4c4c(-c5cc6c(cn5)c5ccccc5n6-c5ccccc5)cccc43)n2)cc1 ; c1ccc(-n2c3ccccc3c3ncc(-c4cccc5c4c4ccccc4n5-c4ccccc4)cc32)cc1 ; c1ccc(-n2c3ccccc3c3ncc(-c4cccc5c4c4ccccc4n5-c4ccc5ccccc5c4)cc32)cc1 ; c1ccc(-c2ccc(-n3c4ccccc4c4c(-c5cnc6c7ccccc7n(-c7ccccc7)c6c5)cccc43)cc2)cc1]

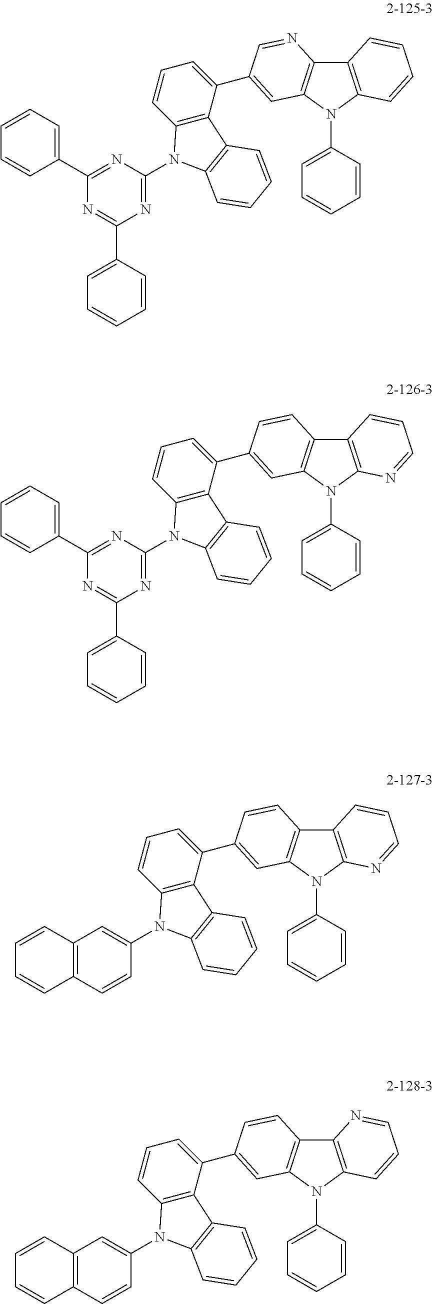 [SMILES: c1ccc(-c2nc(-c3ccccc3)nc(-n3c4ccccc4c4c(-c5cnc6c7ccccc7n(-c7ccccc7)c6c5)cccc43)n2)cc1 ; c1ccc(-c2nc(-c3ccccc3)nc(-n3c4ccccc4c4c(-c5ccc6c7cccnc7n(-c7ccccc7)c6c5)cccc43)n2)cc1 ; c1ccc(-n2c3cc(-c4cccc5c4c4ccccc4n5-c4ccc5ccccc5c4)ccc3c3cccnc32)cc1 ; c1ccc(-n2c3cc(-c4cccc5c4c4ccccc4n5-c4ccc5ccccc5c4)ccc3c3ncccc32)cc1]

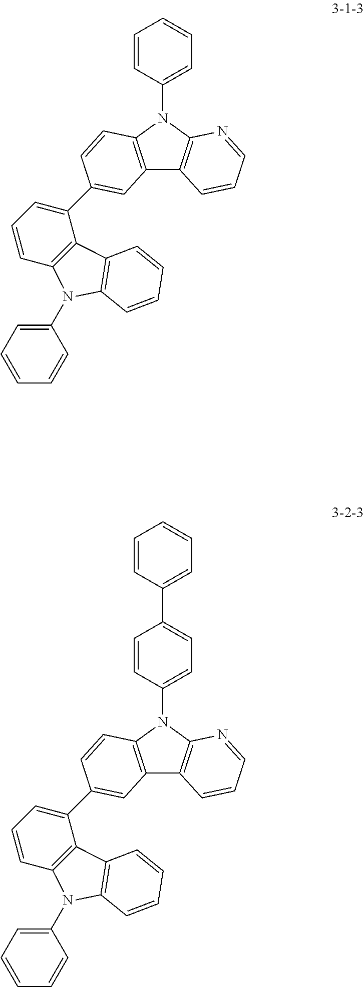 [SMILES: c1ccc(-n2c3ccccc3c3c(-c4ccc5c(c4)c4cccnc4n5-c4ccccc4)cccc32)cc1 ; c1ccc(-c2ccc(-n3c4ccc(-c5cccc6c5c5ccccc5n6-c5ccccc5)cc4c4cccnc43)cc2)cc1]

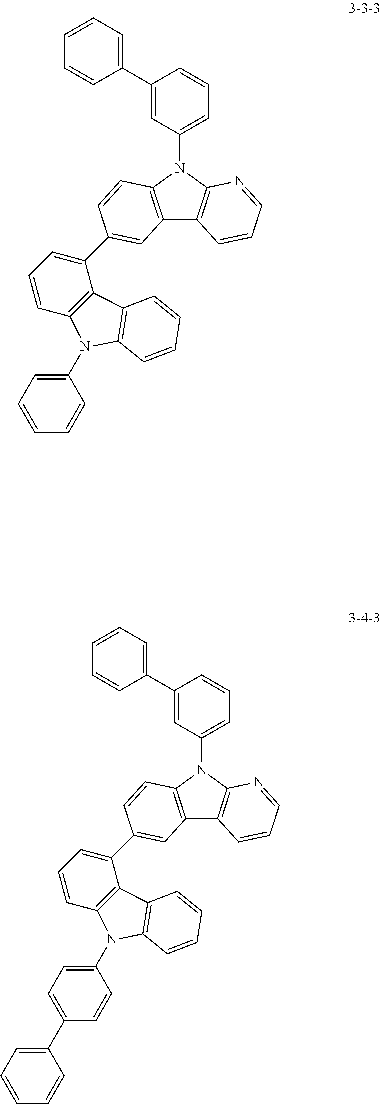 [SMILES: c1ccc(-c2cccc(-n3c4ccc(-c5cccc6c5c5ccccc5n6-c5ccccc5)cc4c4cccnc43)c2)cc1 ; c1ccc(-c2ccc(-n3c4ccccc4c4c(-c5ccc6c(c5)c5cccnc5n6-c5cccc(-c6ccccc6)c5)cccc43)cc2)cc1]

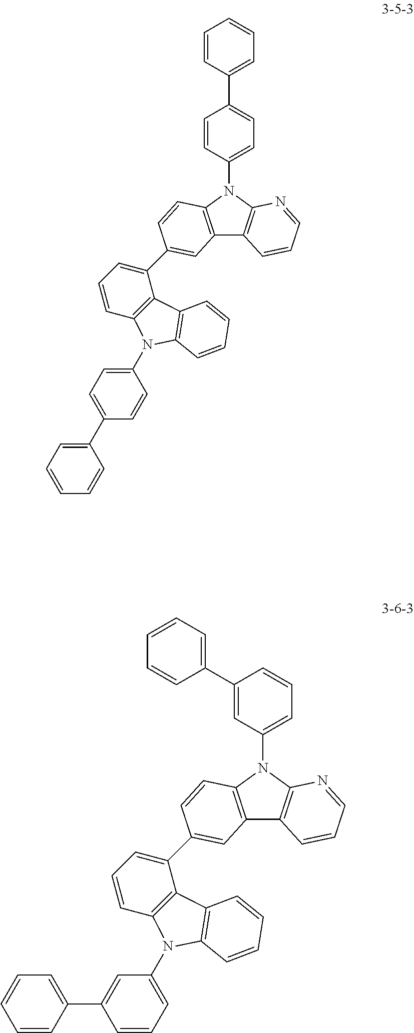 [SMILES: c1ccc(-c2ccc(-n3c4ccccc4c4c(-c5ccc6c(c5)c5cccnc5n6-c5ccc(-c6ccccc6)cc5)cccc43)cc2)cc1 ; c1ccc(-c2cccc(-n3c4ccccc4c4c(-c5ccc6c(c5)c5cccnc5n6-c5cccc(-c6ccccc6)c5)cccc43)c2)cc1]

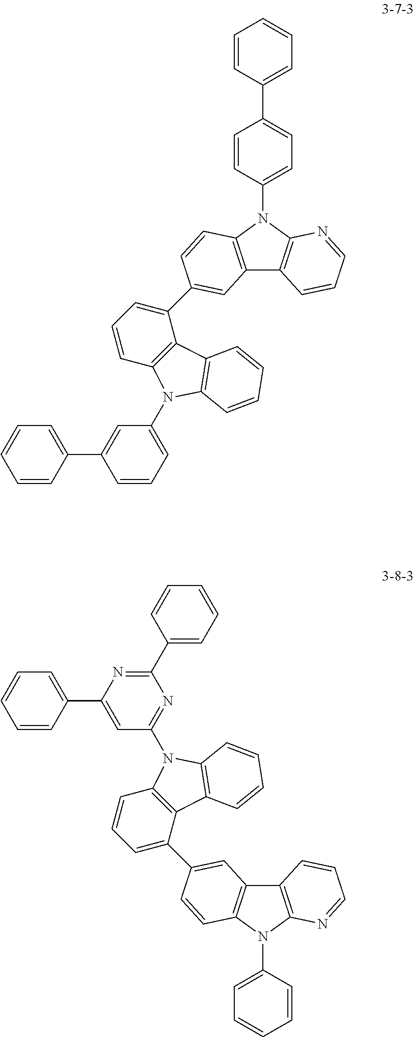 [SMILES: c1ccc(-c2ccc(-n3c4ccc(-c5cccc6c5c5ccccc5n6-c5cccc(-c6ccccc6)c5)cc4c4cccnc43)cc2)cc1 ; c1ccc(-c2cc(-n3c4ccccc4c4c(-c5ccc6c(c5)c5cccnc5n6-c5ccccc5)cccc43)nc(-c3ccccc3)n2)cc1]

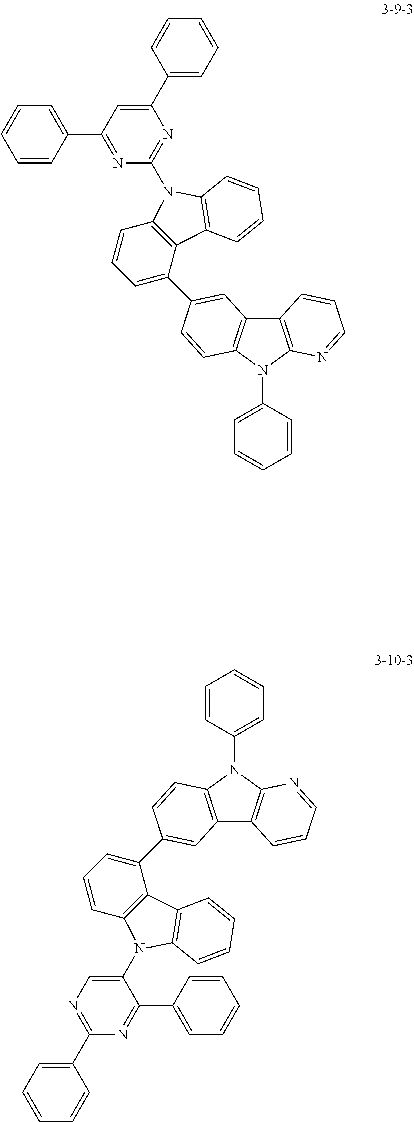 [SMILES: c1ccc(-c2cc(-c3ccccc3)nc(-n3c4ccccc4c4c(-c5ccc6c(c5)c5cccnc5n6-c5ccccc5)cccc43)n2)cc1 ; c1ccc(-c2ncc(-n3c4ccccc4c4c(-c5ccc6c(c5)c5cccnc5n6-c5ccccc5)cccc43)c(-c3ccccc3)n2)cc1]

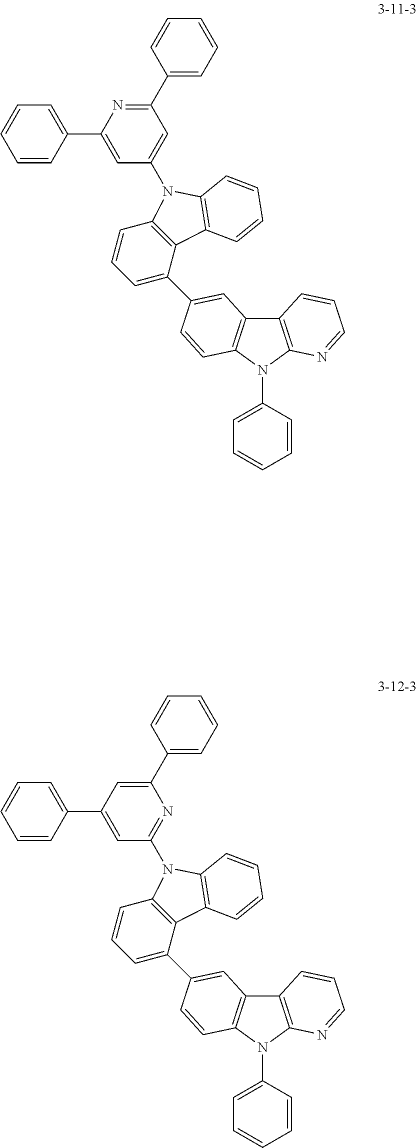 [SMILES: c1ccc(-c2cc(-n3c4ccccc4c4c(-c5ccc6c(c5)c5cccnc5n6-c5ccccc5)cccc43)cc(-c3ccccc3)n2)cc1 ; c1ccc(-c2cc(-c3ccccc3)nc(-n3c4ccccc4c4c(-c5ccc6c(c5)c5cccnc5n6-c5ccccc5)cccc43)c2)cc1]

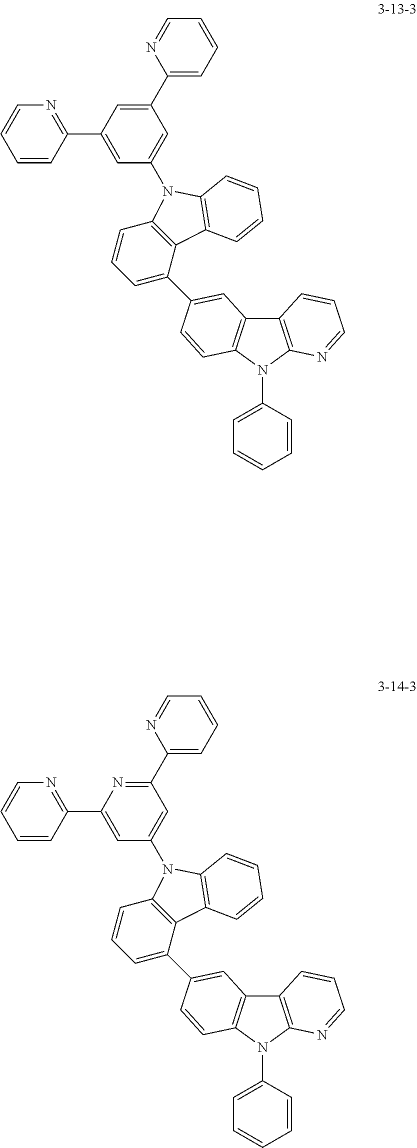 [SMILES: c1ccc(-n2c3ccc(-c4cccc5c4c4ccccc4n5-c4cc(-c5ccccn5)cc(-c5ccccn5)c4)cc3c3cccnc32)cc1 ; c1ccc(-n2c3ccc(-c4cccc5c4c4ccccc4n5-c4cc(-c5ccccn5)nc(-c5ccccn5)c4)cc3c3cccnc32)cc1]

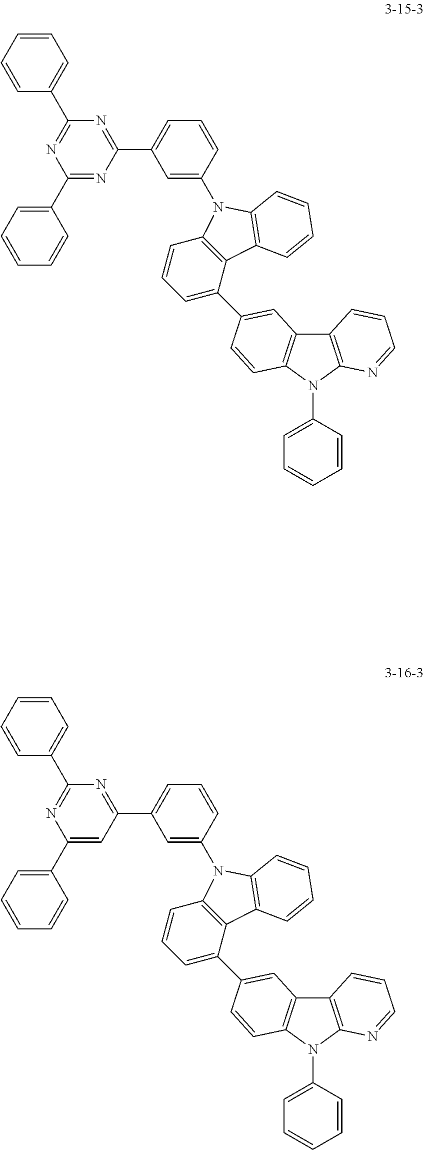 [SMILES: c1ccc(-c2nc(-c3ccccc3)nc(-c3cccc(-n4c5ccccc5c5c(-c6ccc7c(c6)c6cccnc6n7-c6ccccc6)cccc54)c3)n2)cc1 ; c1ccc(-c2cc(-c3cccc(-n4c5ccccc5c5c(-c6ccc7c(c6)c6cccnc6n7-c6ccccc6)cccc54)c3)nc(-c3ccccc3)n2)cc1]

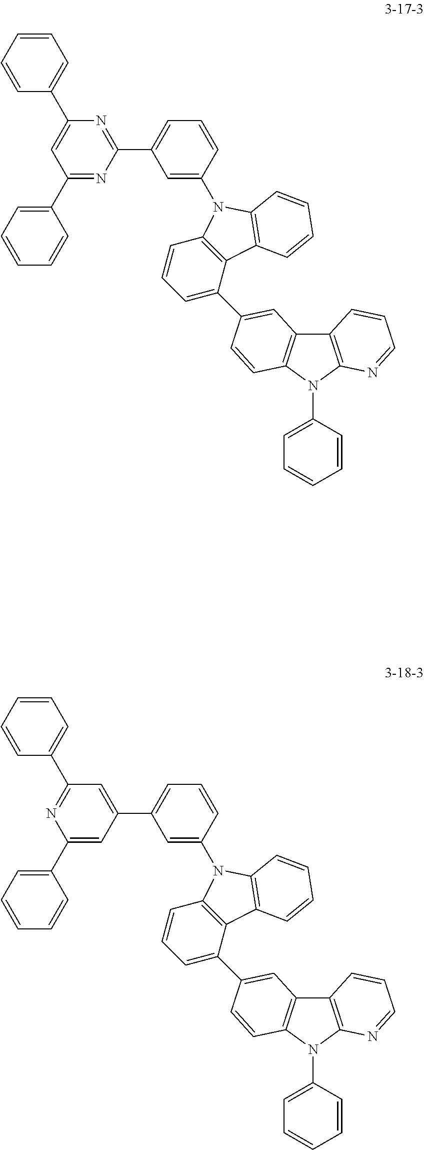 [SMILES: c1ccc(-c2cc(-c3ccccc3)nc(-c3cccc(-n4c5ccccc5c5c(-c6ccc7c(c6)c6cccnc6n7-c6ccccc6)cccc54)c3)n2)cc1 ; c1ccc(-c2cc(-c3cccc(-n4c5ccccc5c5c(-c6ccc7c(c6)c6cccnc6n7-c6ccccc6)cccc54)c3)cc(-c3ccccc3)n2)cc1]

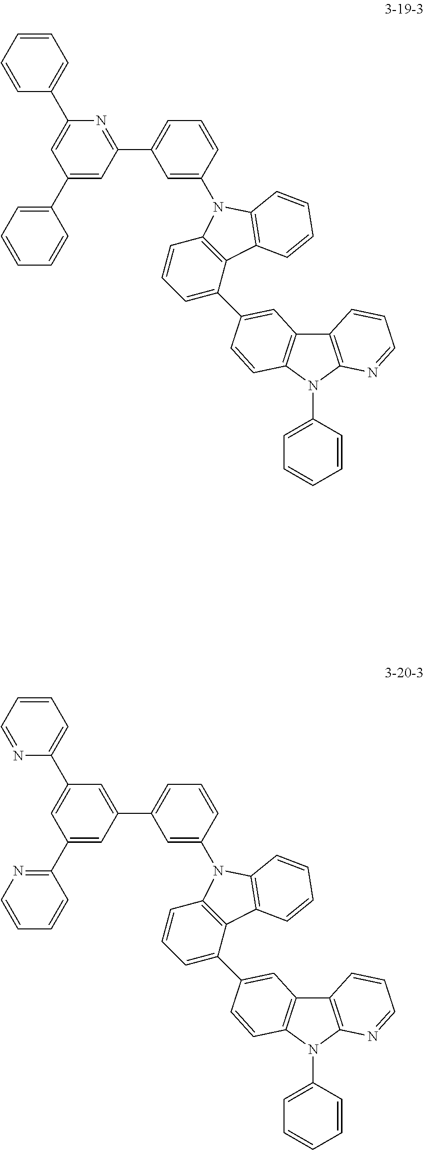 [SMILES: c1ccc(-c2cc(-c3ccccc3)nc(-c3cccc(-n4c5ccccc5c5c(-c6ccc7c(c6)c6cccnc6n7-c6ccccc6)cccc54)c3)c2)cc1 ; c1ccc(-n2c3ccc(-c4cccc5c4c4ccccc4n5-c4cccc(-c5cc(-c6ccccn6)cc(-c6ccccn6)c5)c4)cc3c3cccnc32)cc1]

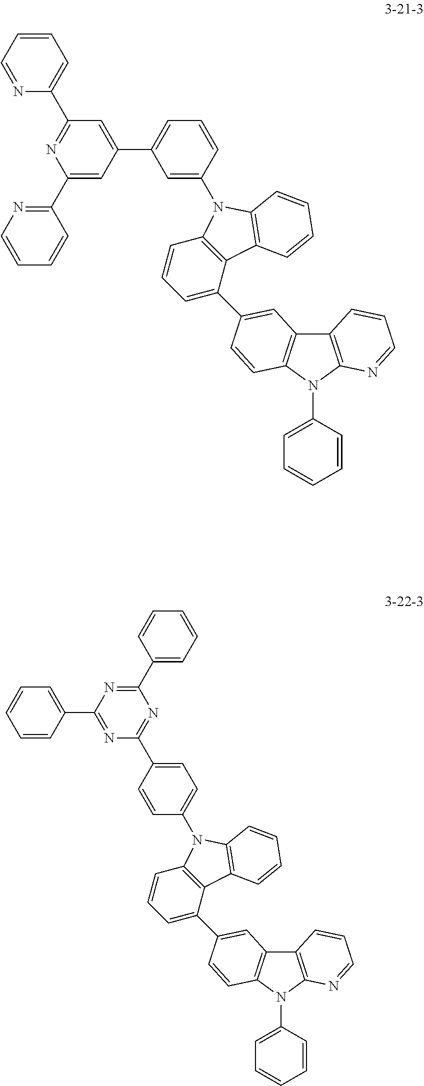 [SMILES: c1ccc(-n2c3ccc(-c4cccc5c4c4ccccc4n5-c4cccc(-c5cc(-c6ccccn6)nc(-c6ccccn6)c5)c4)cc3c3cccnc32)cc1 ; c1ccc(-c2nc(-c3ccccc3)nc(-c3ccc(-n4c5ccccc5c5c(-c6ccc7c(c6)c6cccnc6n7-c6ccccc6)cccc54)cc3)n2)cc1]

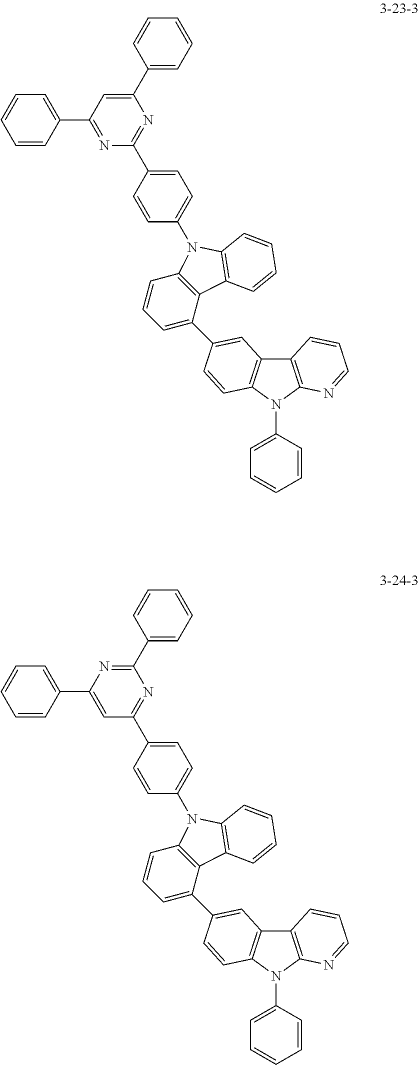 [SMILES: c1ccc(-c2cc(-c3ccccc3)nc(-c3ccc(-n4c5ccccc5c5c(-c6ccc7c(c6)c6cccnc6n7-c6ccccc6)cccc54)cc3)n2)cc1 ; c1ccc(-c2cc(-c3ccc(-n4c5ccccc5c5c(-c6ccc7c(c6)c6cccnc6n7-c6ccccc6)cccc54)cc3)nc(-c3ccccc3)n2)cc1]

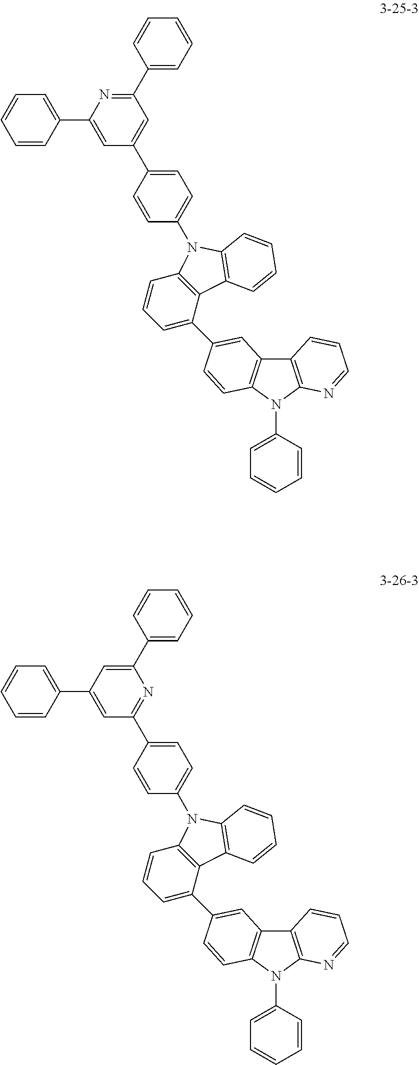 [SMILES: c1ccc(-c2cc(-c3ccc(-n4c5ccccc5c5c(-c6ccc7c(c6)c6cccnc6n7-c6ccccc6)cccc54)cc3)cc(-c3ccccc3)n2)cc1 ; c1ccc(-c2cc(-c3ccccc3)nc(-c3ccc(-n4c5ccccc5c5c(-c6ccc7c(c6)c6cccnc6n7-c6ccccc6)cccc54)cc3)c2)cc1]

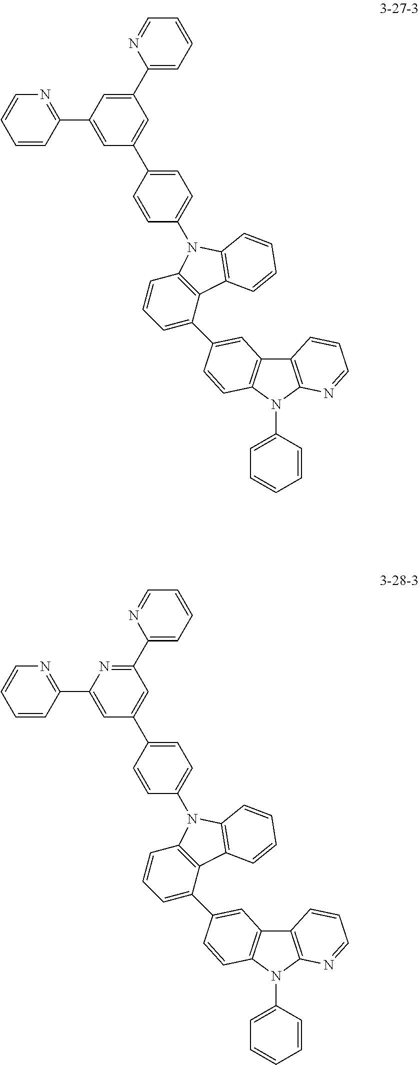 [SMILES: c1ccc(-n2c3ccc(-c4cccc5c4c4ccccc4n5-c4ccc(-c5cc(-c6ccccn6)cc(-c6ccccn6)c5)cc4)cc3c3cccnc32)cc1 ; c1ccc(-n2c3ccc(-c4cccc5c4c4ccccc4n5-c4ccc(-c5cc(-c6ccccn6)nc(-c6ccccn6)c5)cc4)cc3c3cccnc32)cc1]

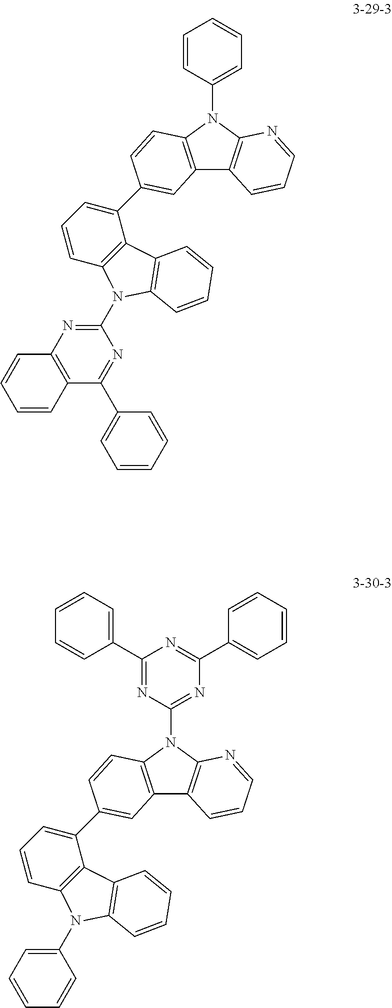 [SMILES: c1ccc(-c2nc(-n3c4ccccc4c4c(-c5ccc6c(c5)c5cccnc5n6-c5ccccc5)cccc43)nc3ccccc23)cc1 ; c1ccc(-c2nc(-c3ccccc3)nc(-n3c4ccc(-c5cccc6c5c5ccccc5n6-c5ccccc5)cc4c4cccnc43)n2)cc1]

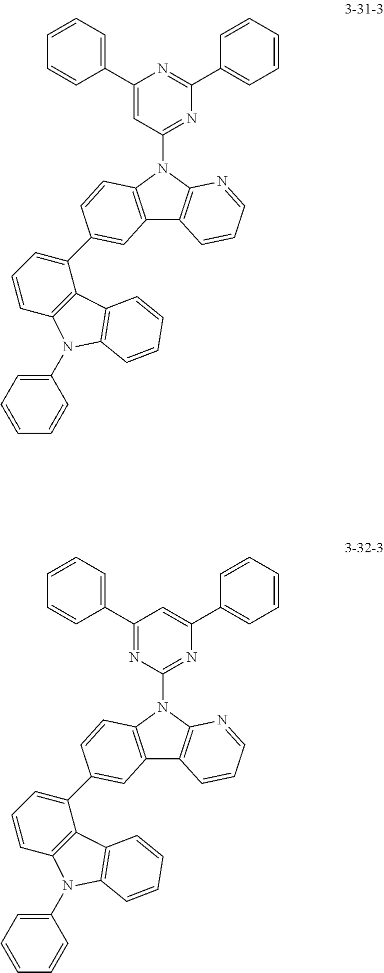 [SMILES: c1ccc(-c2cc(-n3c4ccc(-c5cccc6c5c5ccccc5n6-c5ccccc5)cc4c4cccnc43)nc(-c3ccccc3)n2)cc1 ; c1ccc(-c2cc(-c3ccccc3)nc(-n3c4ccc(-c5cccc6c5c5ccccc5n6-c5ccccc5)cc4c4cccnc43)n2)cc1]

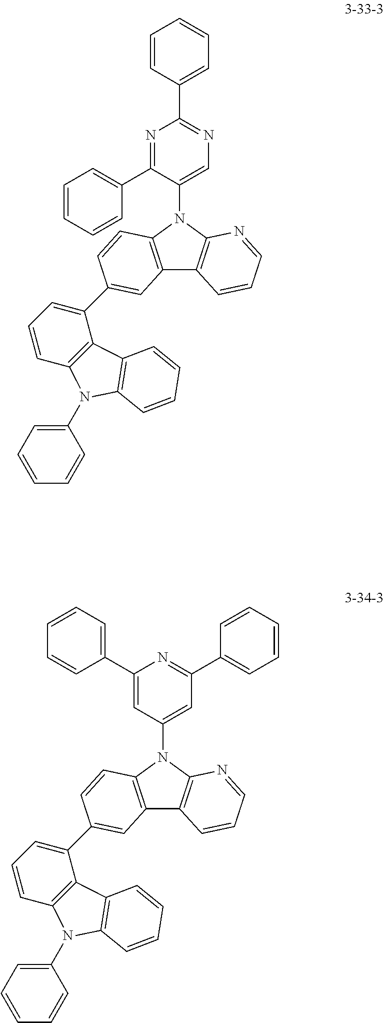 [SMILES: c1ccc(-c2ncc(-n3c4ccc(-c5cccc6c5c5ccccc5n6-c5ccccc5)cc4c4cccnc43)c(-c3ccccc3)n2)cc1 ; c1ccc(-c2cc(-n3c4ccc(-c5cccc6c5c5ccccc5n6-c5ccccc5)cc4c4cccnc43)cc(-c3ccccc3)n2)cc1]

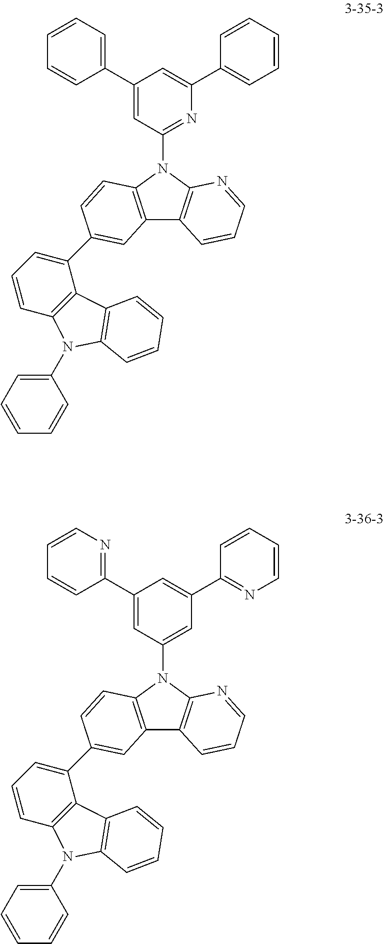 [SMILES: c1ccc(-c2cc(-c3ccccc3)nc(-n3c4ccc(-c5cccc6c5c5ccccc5n6-c5ccccc5)cc4c4cccnc43)c2)cc1 ; c1ccc(-n2c3ccccc3c3c(-c4ccc5c(c4)c4cccnc4n5-c4cc(-c5ccccn5)cc(-c5ccccn5)c4)cccc32)cc1]

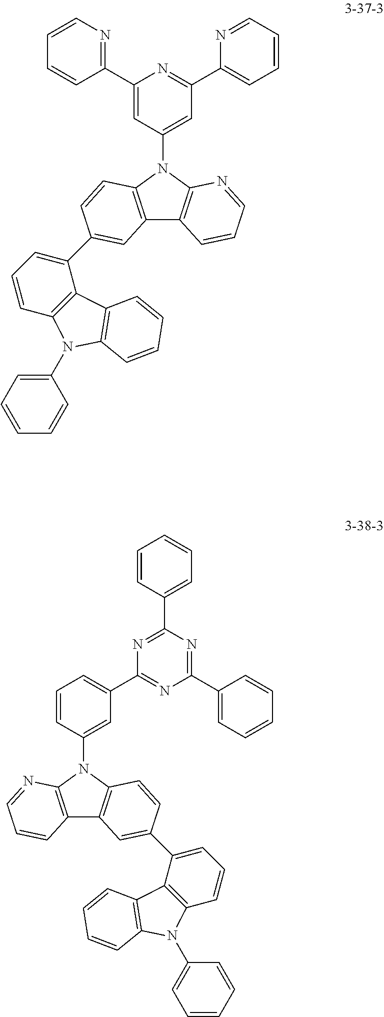 [SMILES: c1ccc(-n2c3ccccc3c3c(-c4ccc5c(c4)c4cccnc4n5-c4cc(-c5ccccn5)nc(-c5ccccn5)c4)cccc32)cc1 ; c1ccc(-c2nc(-c3ccccc3)nc(-c3cccc(-n4c5ccc(-c6cccc7c6c6ccccc6n7-c6ccccc6)cc5c5cccnc54)c3)n2)cc1]

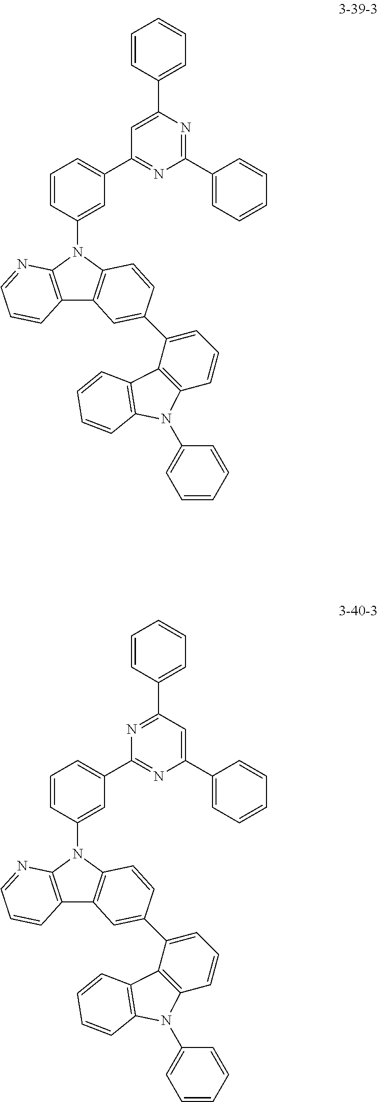 [SMILES: c1ccc(-c2cc(-c3cccc(-n4c5ccc(-c6cccc7c6c6ccccc6n7-c6ccccc6)cc5c5cccnc54)c3)nc(-c3ccccc3)n2)cc1 ; c1ccc(-c2cc(-c3ccccc3)nc(-c3cccc(-n4c5ccc(-c6cccc7c6c6ccccc6n7-c6ccccc6)cc5c5cccnc54)c3)n2)cc1]

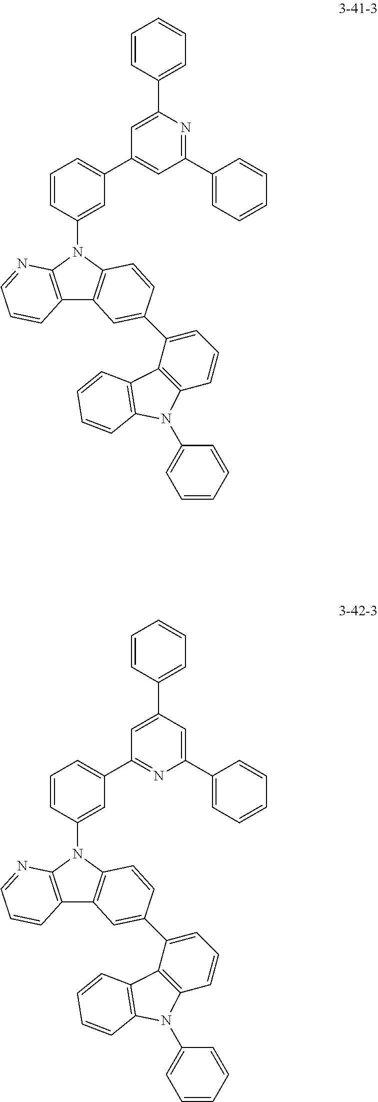 [SMILES: c1ccc(-c2cc(-c3cccc(-n4c5ccc(-c6cccc7c6c6ccccc6n7-c6ccccc6)cc5c5cccnc54)c3)cc(-c3ccccc3)n2)cc1 ; c1ccc(-c2cc(-c3ccccc3)nc(-c3cccc(-n4c5ccc(-c6cccc7c6c6ccccc6n7-c6ccccc6)cc5c5cccnc54)c3)c2)cc1]

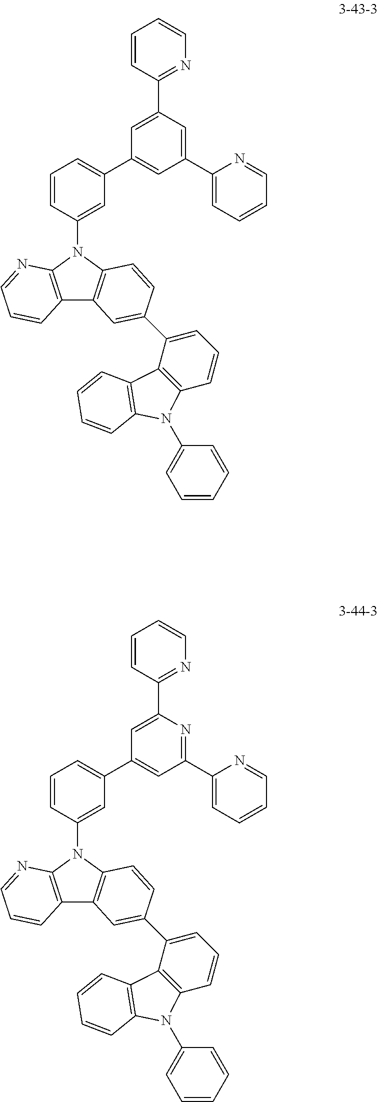 [SMILES: c1ccc(-n2c3ccccc3c3c(-c4ccc5c(c4)c4cccnc4n5-c4cccc(-c5cc(-c6ccccn6)cc(-c6ccccn6)c5)c4)cccc32)cc1 ; c1ccc(-n2c3ccccc3c3c(-c4ccc5c(c4)c4cccnc4n5-c4cccc(-c5cc(-c6ccccn6)nc(-c6ccccn6)c5)c4)cccc32)cc1]

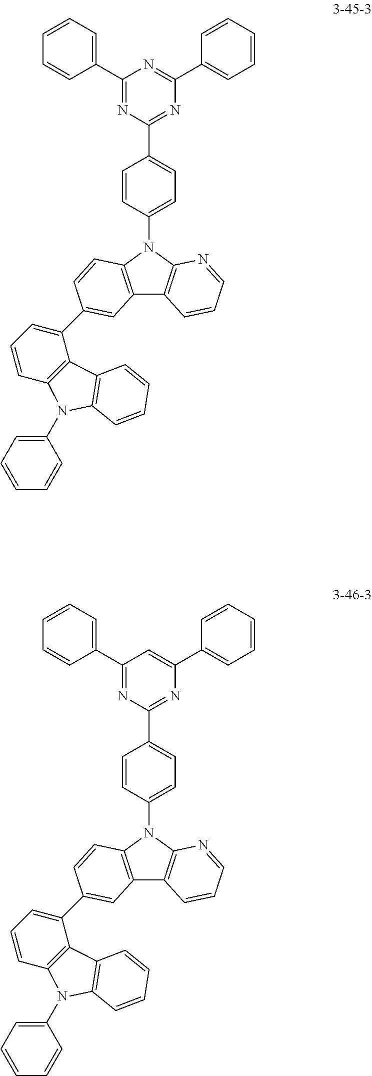 [SMILES: c1ccc(-c2nc(-c3ccccc3)nc(-c3ccc(-n4c5ccc(-c6cccc7c6c6ccccc6n7-c6ccccc6)cc5c5cccnc54)cc3)n2)cc1 ; c1ccc(-c2cc(-c3ccccc3)nc(-c3ccc(-n4c5ccc(-c6cccc7c6c6ccccc6n7-c6ccccc6)cc5c5cccnc54)cc3)n2)cc1]

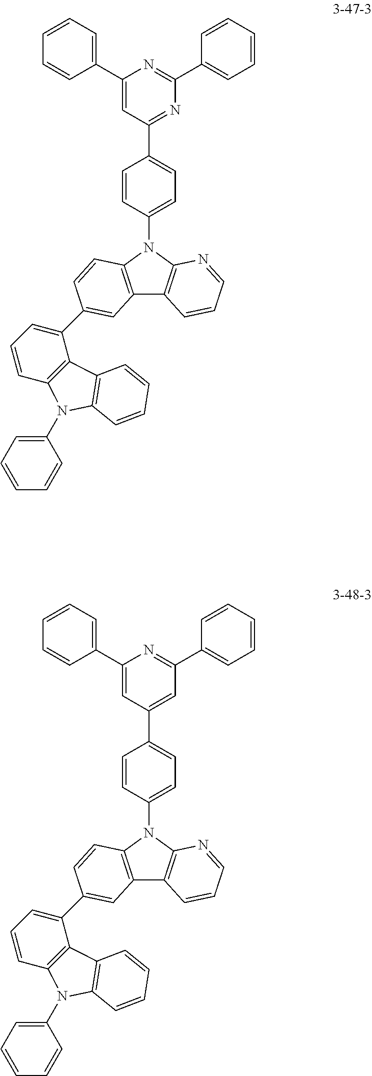 [SMILES: c1ccc(-c2cc(-c3ccc(-n4c5ccc(-c6cccc7c6c6ccccc6n7-c6ccccc6)cc5c5cccnc54)cc3)nc(-c3ccccc3)n2)cc1 ; c1ccc(-c2cc(-c3ccc(-n4c5ccc(-c6cccc7c6c6ccccc6n7-c6ccccc6)cc5c5cccnc54)cc3)cc(-c3ccccc3)n2)cc1]

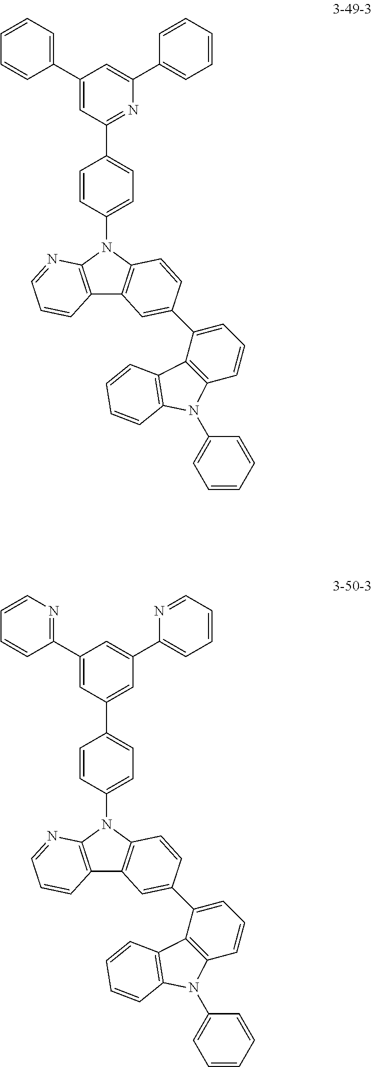 [SMILES: c1ccc(-c2cc(-c3ccccc3)nc(-c3ccc(-n4c5ccc(-c6cccc7c6c6ccccc6n7-c6ccccc6)cc5c5cccnc54)cc3)c2)cc1 ; c1ccc(-n2c3ccccc3c3c(-c4ccc5c(c4)c4cccnc4n5-c4ccc(-c5cc(-c6ccccn6)cc(-c6ccccn6)c5)cc4)cccc32)cc1]

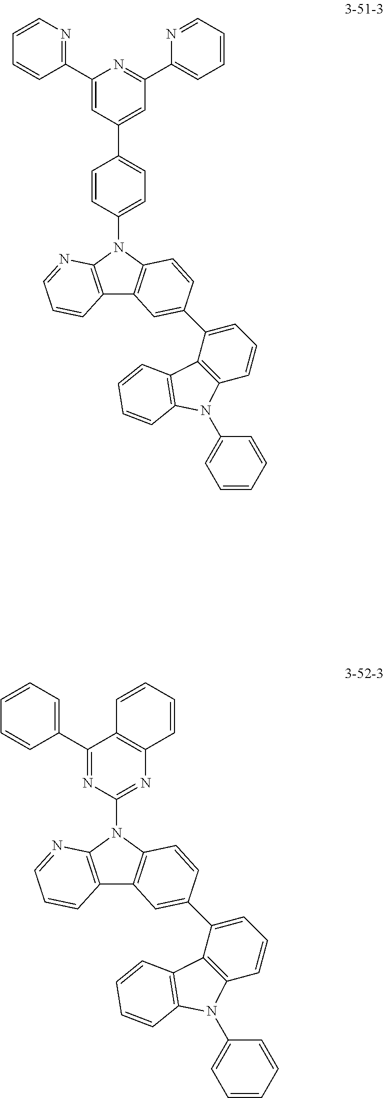 [SMILES: c1ccc(-n2c3ccccc3c3c(-c4ccc5c(c4)c4cccnc4n5-c4ccc(-c5cc(-c6ccccn6)nc(-c6ccccn6)c5)cc4)cccc32)cc1 ; c1ccc(-c2nc(-n3c4ccc(-c5cccc6c5c5ccccc5n6-c5ccccc5)cc4c4cccnc43)nc3ccccc23)cc1]

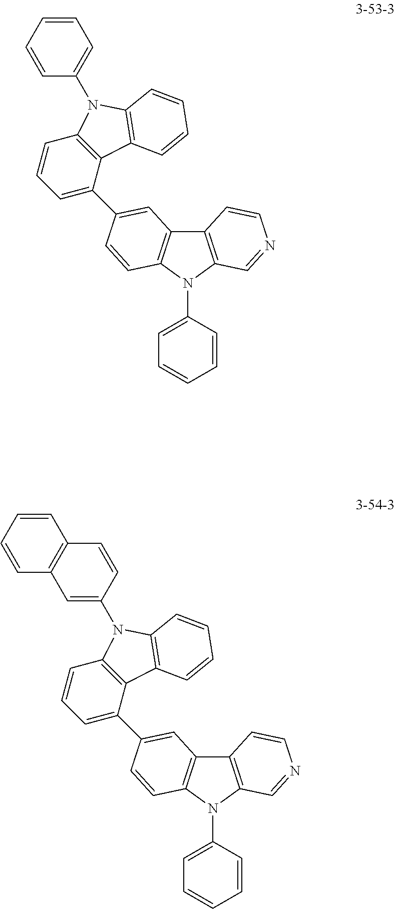 [SMILES: c1ccc(-n2c3ccc(-c4cccc5c4c4ccccc4n5-c4ccccc4)cc3c3ccncc32)cc1 ; c1ccc(-n2c3ccc(-c4cccc5c4c4ccccc4n5-c4ccc5ccccc5c4)cc3c3ccncc32)cc1]

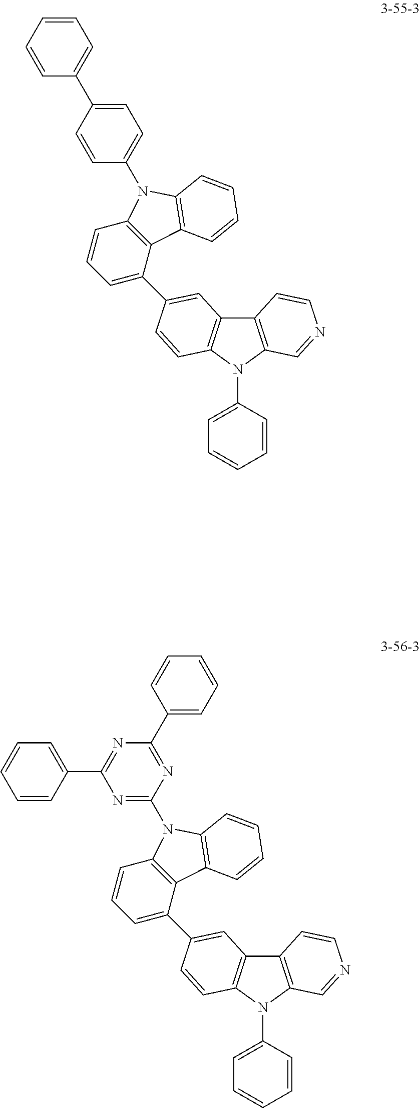 [SMILES: c1ccc(-c2ccc(-n3c4ccccc4c4c(-c5ccc6c(c5)c5ccncc5n6-c5ccccc5)cccc43)cc2)cc1 ; c1ccc(-c2nc(-c3ccccc3)nc(-n3c4ccccc4c4c(-c5ccc6c(c5)c5ccncc5n6-c5ccccc5)cccc43)n2)cc1]

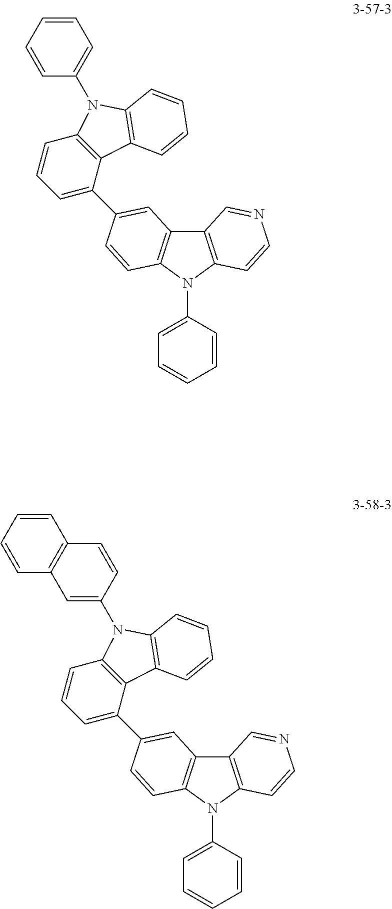 [SMILES: c1ccc(-n2c3ccncc3c3cc(-c4cccc5c4c4ccccc4n5-c4ccccc4)ccc32)cc1 ; c1ccc(-n2c3ccncc3c3cc(-c4cccc5c4c4ccccc4n5-c4ccc5ccccc5c4)ccc32)cc1]

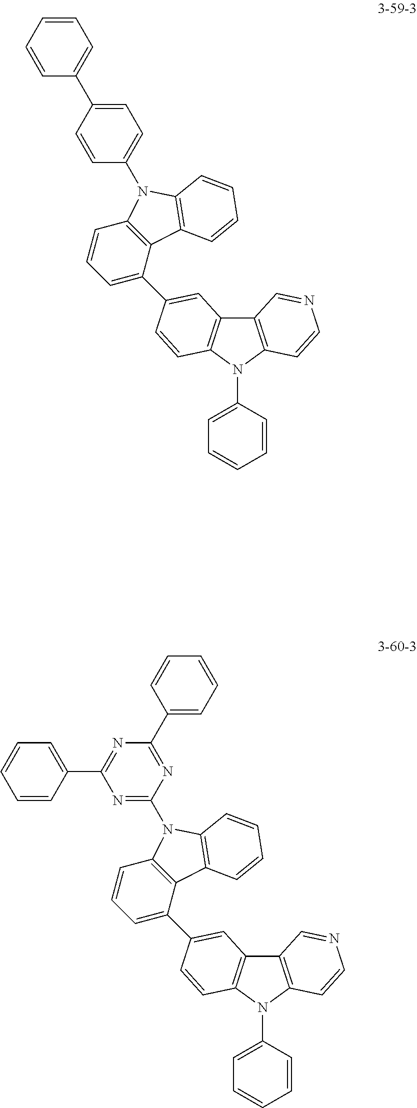 [SMILES: c1ccc(-c2ccc(-n3c4ccccc4c4c(-c5ccc6c(c5)c5cnccc5n6-c5ccccc5)cccc43)cc2)cc1 ; c1ccc(-c2nc(-c3ccccc3)nc(-n3c4ccccc4c4c(-c5ccc6c(c5)c5cnccc5n6-c5ccccc5)cccc43)n2)cc1]

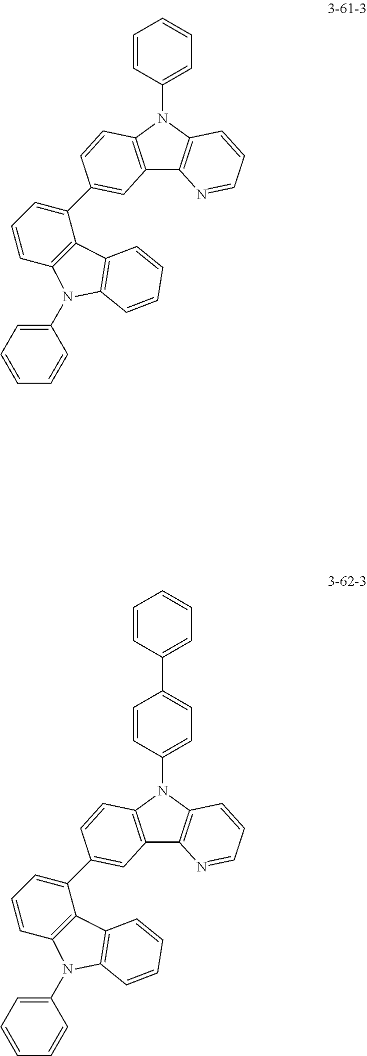 [SMILES: c1ccc(-n2c3ccc(-c4cccc5c4c4ccccc4n5-c4ccccc4)cc3c3ncccc32)cc1 ; c1ccc(-c2ccc(-n3c4ccc(-c5cccc6c5c5ccccc5n6-c5ccccc5)cc4c4ncccc43)cc2)cc1]

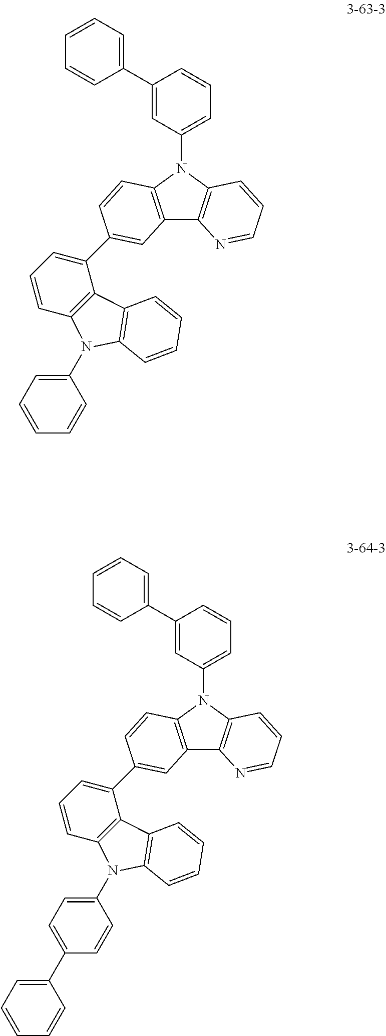 [SMILES: c1ccc(-c2cccc(-n3c4ccc(-c5cccc6c5c5ccccc5n6-c5ccccc5)cc4c4ncccc43)c2)cc1 ; c1ccc(-c2ccc(-n3c4ccccc4c4c(-c5ccc6c(c5)c5ncccc5n6-c5cccc(-c6ccccc6)c5)cccc43)cc2)cc1]

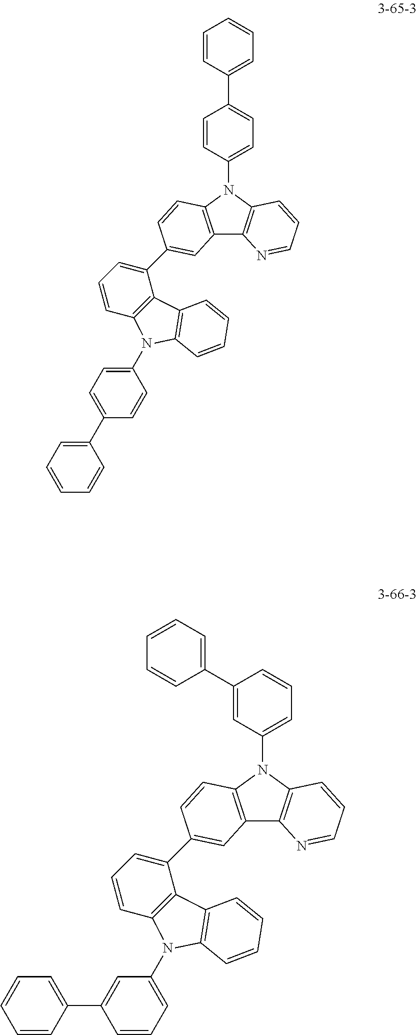 [SMILES: c1ccc(-c2ccc(-n3c4ccc(-c5cccc6c5c5ccccc5n6-c5ccc(-c6ccccc6)cc5)cc4c4ncccc43)cc2)cc1 ; c1ccc(-c2cccc(-n3c4ccc(-c5cccc6c5c5ccccc5n6-c5cccc(-c6ccccc6)c5)cc4c4ncccc43)c2)cc1]

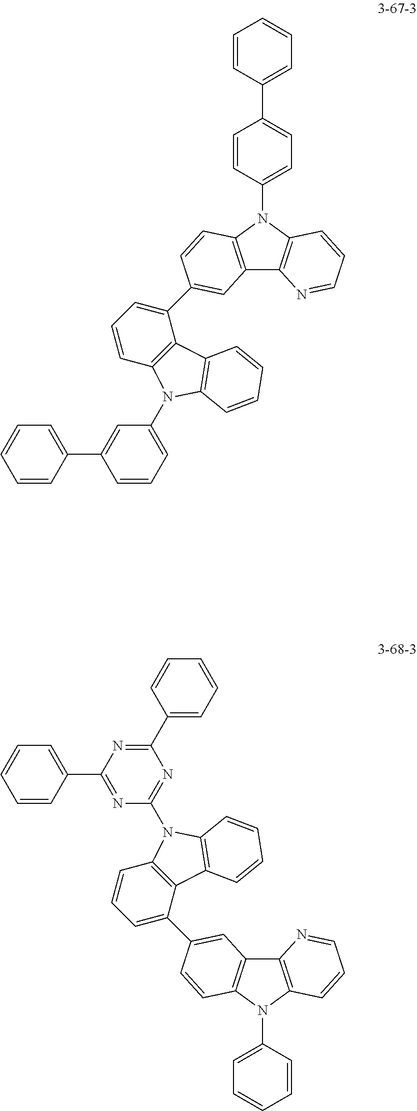 [SMILES: c1ccc(-c2ccc(-n3c4ccc(-c5cccc6c5c5ccccc5n6-c5cccc(-c6ccccc6)c5)cc4c4ncccc43)cc2)cc1 ; c1ccc(-c2nc(-c3ccccc3)nc(-n3c4ccccc4c4c(-c5ccc6c(c5)c5ncccc5n6-c5ccccc5)cccc43)n2)cc1]

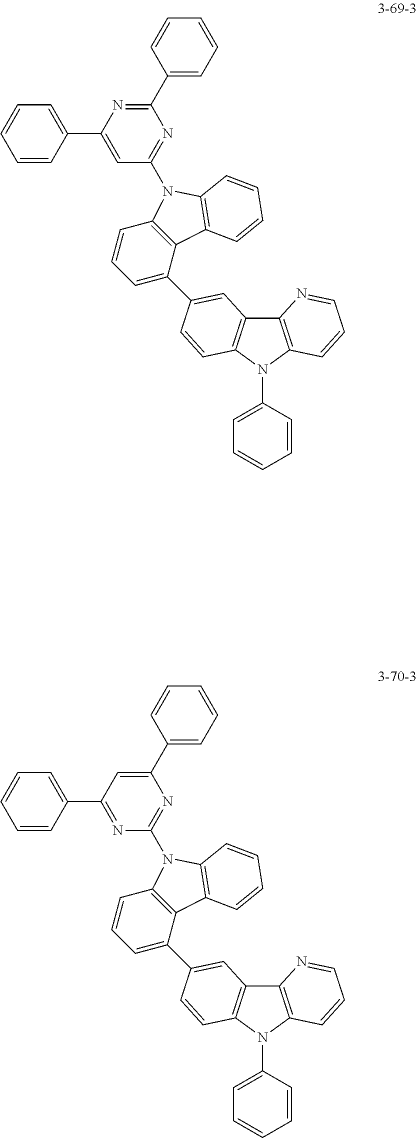 [SMILES: c1ccc(-c2cc(-n3c4ccccc4c4c(-c5ccc6c(c5)c5ncccc5n6-c5ccccc5)cccc43)nc(-c3ccccc3)n2)cc1 ; c1ccc(-c2cc(-c3ccccc3)nc(-n3c4ccccc4c4c(-c5ccc6c(c5)c5ncccc5n6-c5ccccc5)cccc43)n2)cc1]

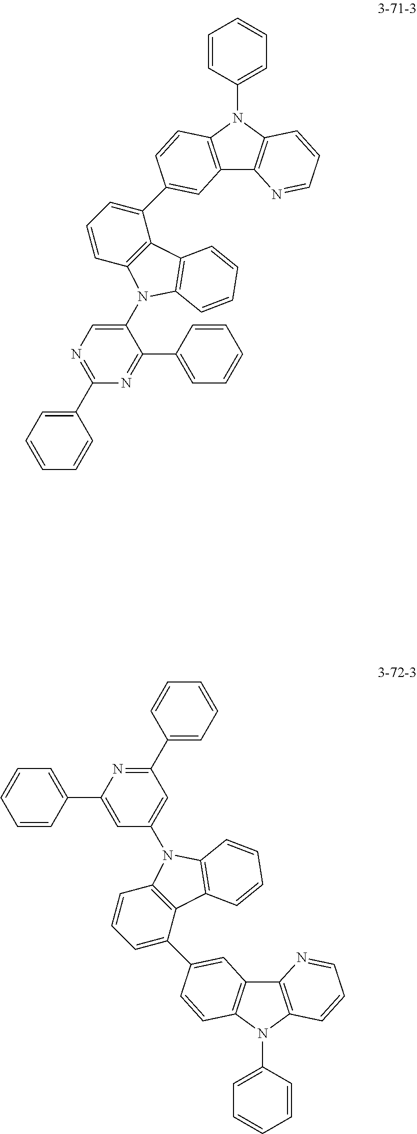 [SMILES: c1ccc(-c2ncc(-n3c4ccccc4c4c(-c5ccc6c(c5)c5ncccc5n6-c5ccccc5)cccc43)c(-c3ccccc3)n2)cc1 ; c1ccc(-c2cc(-n3c4ccccc4c4c(-c5ccc6c(c5)c5ncccc5n6-c5ccccc5)cccc43)cc(-c3ccccc3)n2)cc1]

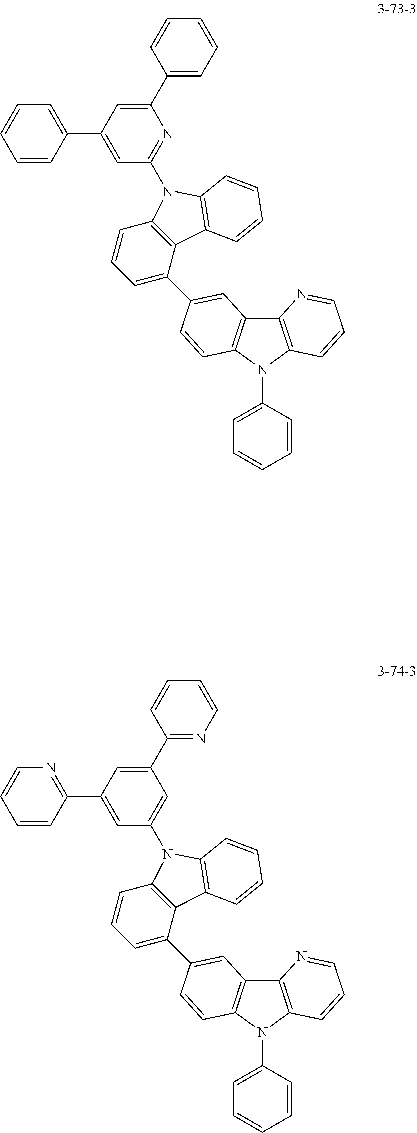 [SMILES: c1ccc(-c2cc(-c3ccccc3)nc(-n3c4ccccc4c4c(-c5ccc6c(c5)c5ncccc5n6-c5ccccc5)cccc43)c2)cc1 ; c1ccc(-n2c3ccc(-c4cccc5c4c4ccccc4n5-c4cc(-c5ccccn5)cc(-c5ccccn5)c4)cc3c3ncccc32)cc1]

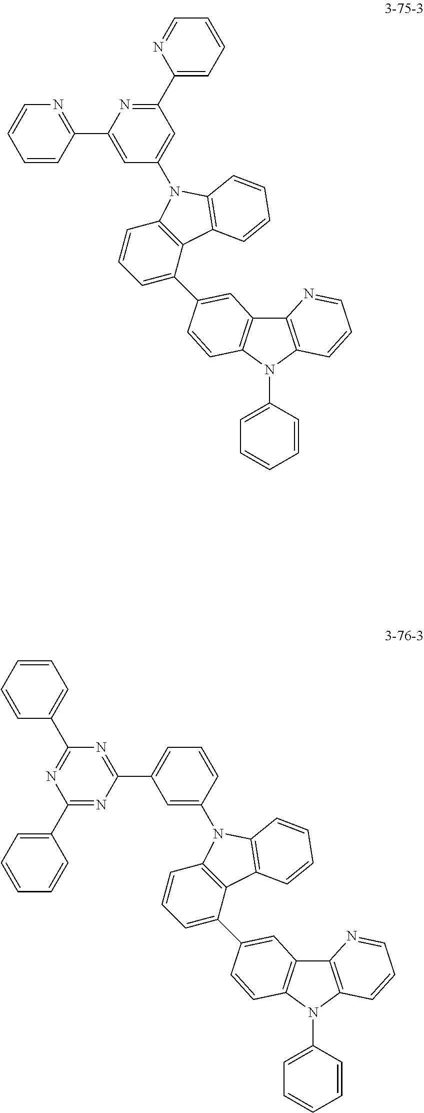 [SMILES: c1ccc(-n2c3ccc(-c4cccc5c4c4ccccc4n5-c4cc(-c5ccccn5)nc(-c5ccccn5)c4)cc3c3ncccc32)cc1 ; c1ccc(-c2nc(-c3ccccc3)nc(-c3cccc(-n4c5ccccc5c5c(-c6ccc7c(c6)c6ncccc6n7-c6ccccc6)cccc54)c3)n2)cc1]

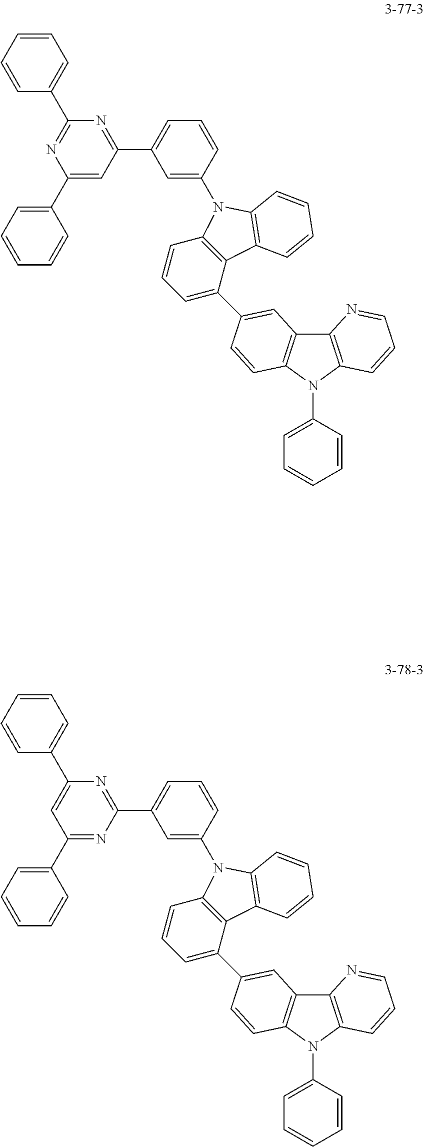 [SMILES: c1ccc(-c2cc(-c3cccc(-n4c5ccccc5c5c(-c6ccc7c(c6)c6ncccc6n7-c6ccccc6)cccc54)c3)nc(-c3ccccc3)n2)cc1 ; c1ccc(-c2cc(-c3ccccc3)nc(-c3cccc(-n4c5ccccc5c5c(-c6ccc7c(c6)c6ncccc6n7-c6ccccc6)cccc54)c3)n2)cc1]

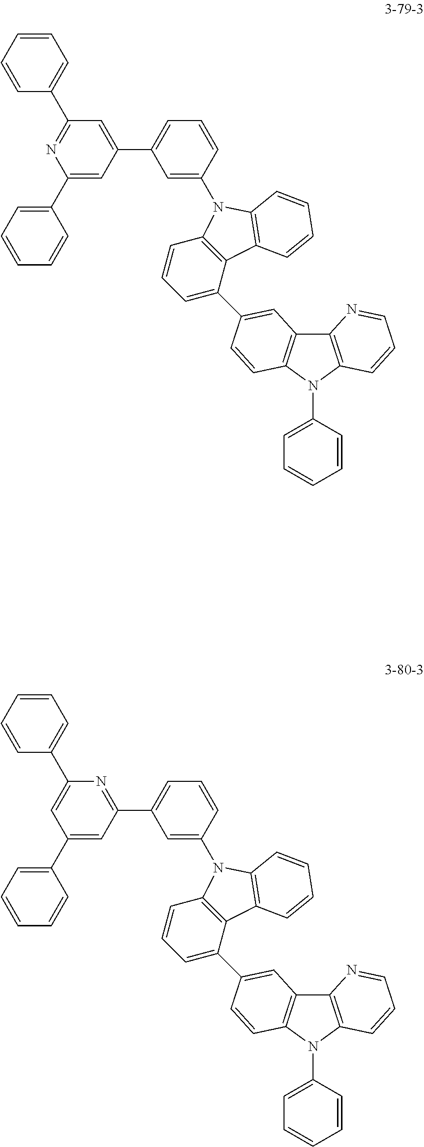 [SMILES: c1ccc(-c2cc(-c3cccc(-n4c5ccccc5c5c(-c6ccc7c(c6)c6ncccc6n7-c6ccccc6)cccc54)c3)cc(-c3ccccc3)n2)cc1 ; c1ccc(-c2cc(-c3ccccc3)nc(-c3cccc(-n4c5ccccc5c5c(-c6ccc7c(c6)c6ncccc6n7-c6ccccc6)cccc54)c3)c2)cc1]

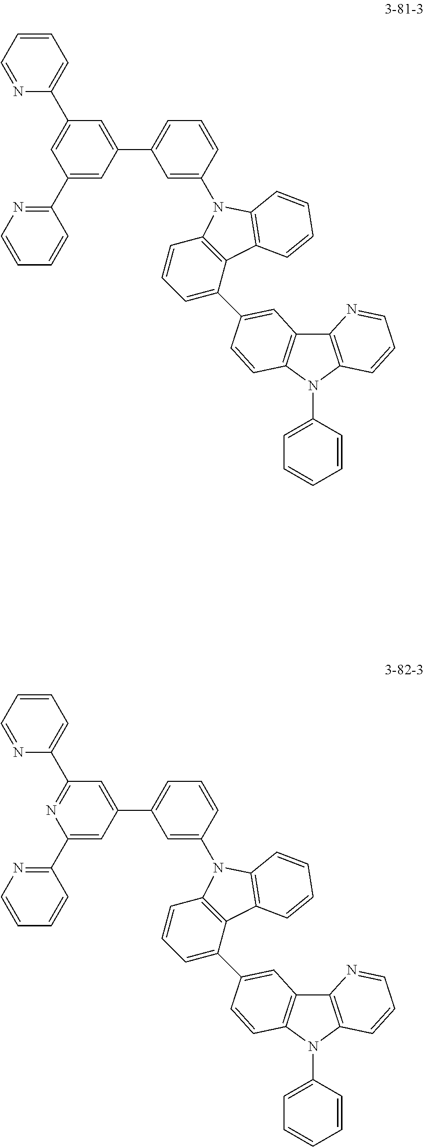 [SMILES: c1ccc(-n2c3ccc(-c4cccc5c4c4ccccc4n5-c4cccc(-c5cc(-c6ccccn6)cc(-c6ccccn6)c5)c4)cc3c3ncccc32)cc1 ; c1ccc(-n2c3ccc(-c4cccc5c4c4ccccc4n5-c4cccc(-c5cc(-c6ccccn6)nc(-c6ccccn6)c5)c4)cc3c3ncccc32)cc1]

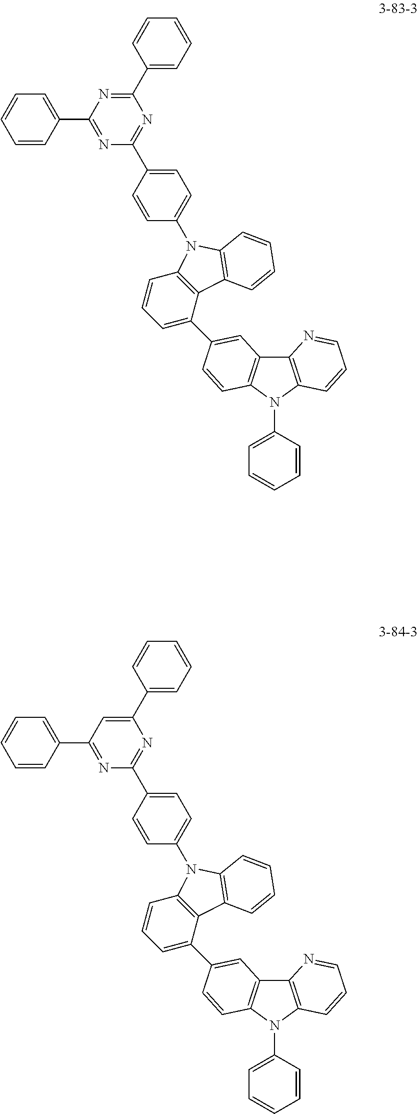 [SMILES: c1ccc(-c2nc(-c3ccccc3)nc(-c3ccc(-n4c5ccccc5c5c(-c6ccc7c(c6)c6ncccc6n7-c6ccccc6)cccc54)cc3)n2)cc1 ; c1ccc(-c2cc(-c3ccccc3)nc(-c3ccc(-n4c5ccccc5c5c(-c6ccc7c(c6)c6ncccc6n7-c6ccccc6)cccc54)cc3)n2)cc1]

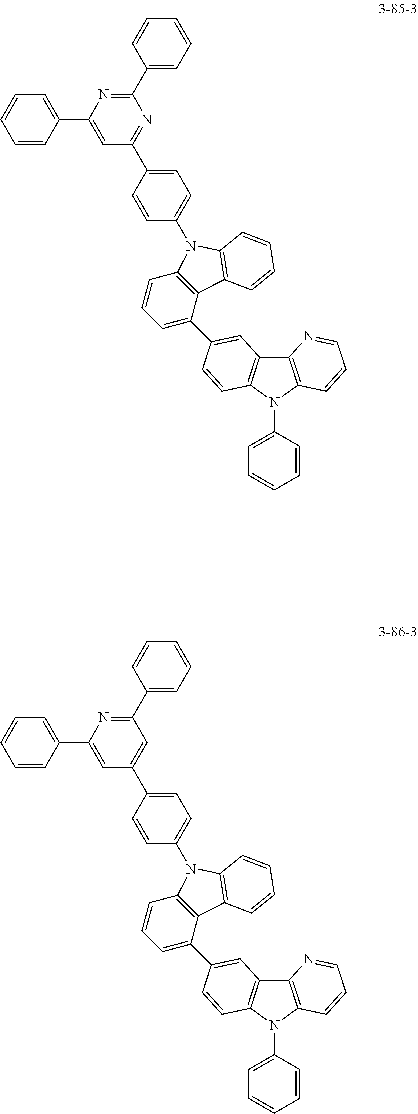 [SMILES: c1ccc(-c2cc(-c3ccc(-n4c5ccccc5c5c(-c6ccc7c(c6)c6ncccc6n7-c6ccccc6)cccc54)cc3)nc(-c3ccccc3)n2)cc1 ; c1ccc(-c2cc(-c3ccc(-n4c5ccccc5c5c(-c6ccc7c(c6)c6ncccc6n7-c6ccccc6)cccc54)cc3)cc(-c3ccccc3)n2)cc1]

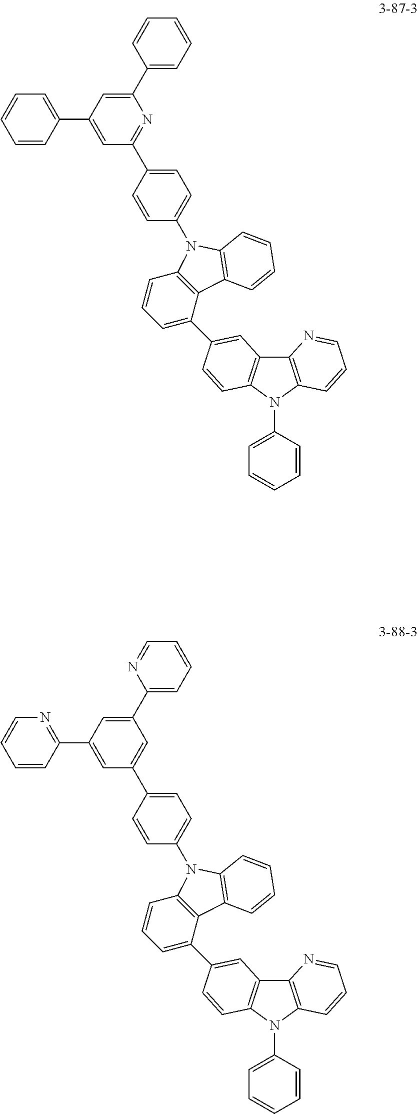 [SMILES: c1ccc(-c2cc(-c3ccccc3)nc(-c3ccc(-n4c5ccccc5c5c(-c6ccc7c(c6)c6ncccc6n7-c6ccccc6)cccc54)cc3)c2)cc1 ; c1ccc(-n2c3ccc(-c4cccc5c4c4ccccc4n5-c4ccc(-c5cc(-c6ccccn6)cc(-c6ccccn6)c5)cc4)cc3c3ncccc32)cc1]

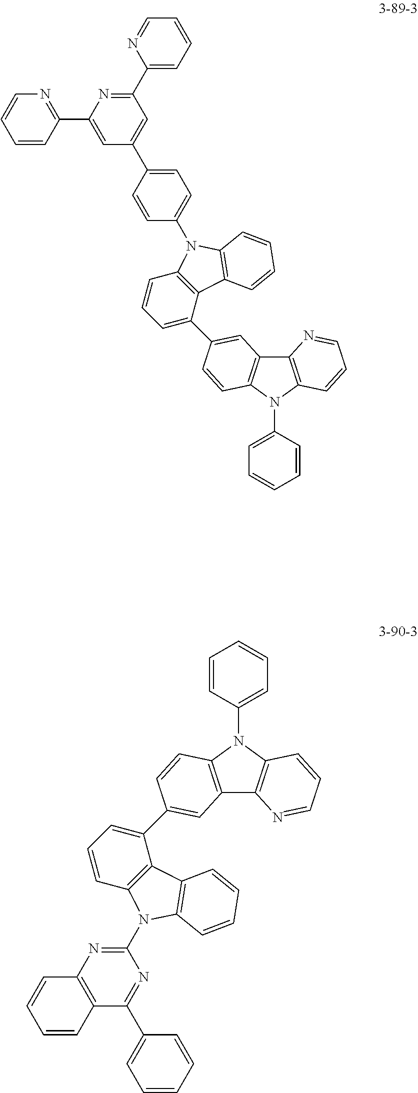 [SMILES: c1ccc(-n2c3ccc(-c4cccc5c4c4ccccc4n5-c4ccc(-c5cc(-c6ccccn6)nc(-c6ccccn6)c5)cc4)cc3c3ncccc32)cc1 ; c1ccc(-c2nc(-n3c4ccccc4c4c(-c5ccc6c(c5)c5ncccc5n6-c5ccccc5)cccc43)nc3ccccc23)cc1]

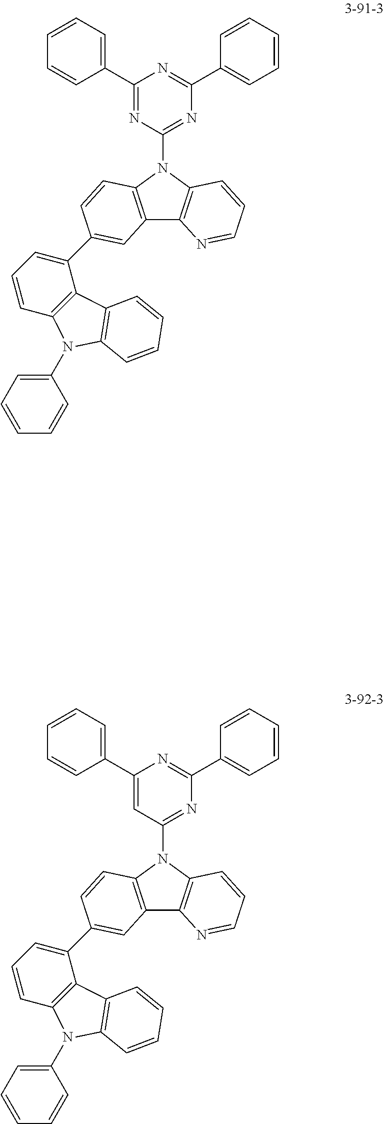 [SMILES: c1ccc(-c2nc(-c3ccccc3)nc(-n3c4ccc(-c5cccc6c5c5ccccc5n6-c5ccccc5)cc4c4ncccc43)n2)cc1 ; c1ccc(-c2cc(-n3c4ccc(-c5cccc6c5c5ccccc5n6-c5ccccc5)cc4c4ncccc43)nc(-c3ccccc3)n2)cc1]

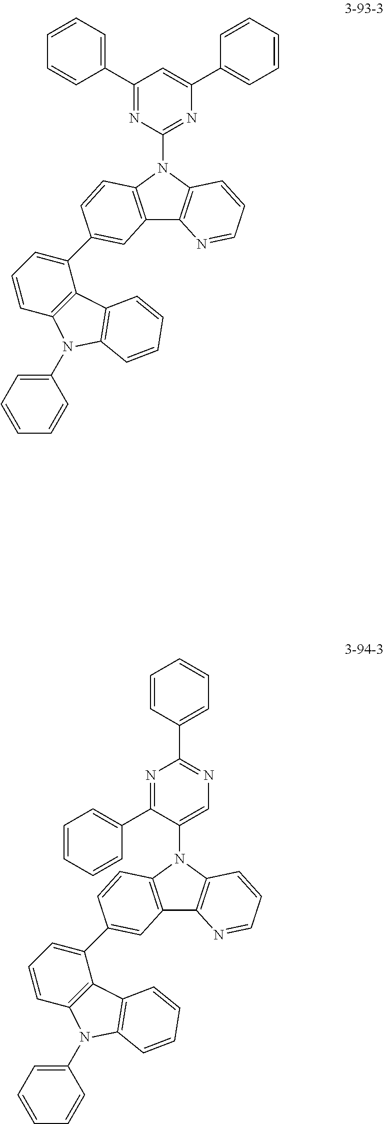 [SMILES: c1ccc(-c2cc(-c3ccccc3)nc(-n3c4ccc(-c5cccc6c5c5ccccc5n6-c5ccccc5)cc4c4ncccc43)n2)cc1 ; c1ccc(-c2ncc(-n3c4ccc(-c5cccc6c5c5ccccc5n6-c5ccccc5)cc4c4ncccc43)c(-c3ccccc3)n2)cc1]

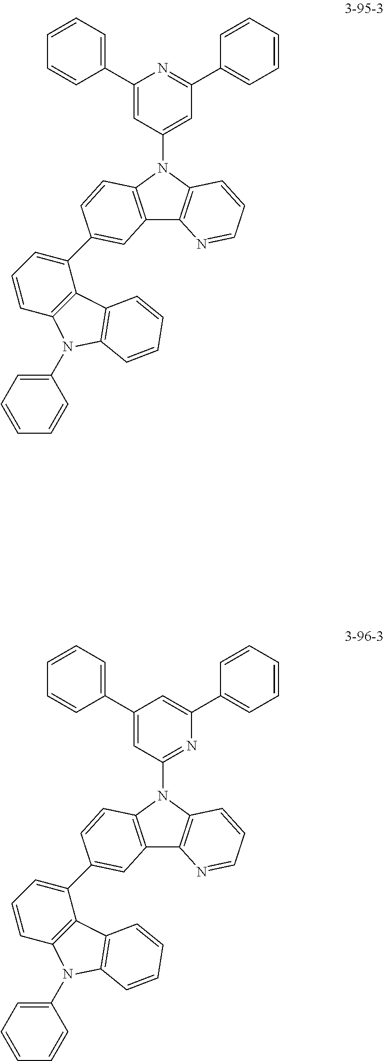 [SMILES: c1ccc(-c2cc(-n3c4ccc(-c5cccc6c5c5ccccc5n6-c5ccccc5)cc4c4ncccc43)cc(-c3ccccc3)n2)cc1 ; c1ccc(-c2cc(-c3ccccc3)nc(-n3c4ccc(-c5cccc6c5c5ccccc5n6-c5ccccc5)cc4c4ncccc43)c2)cc1]

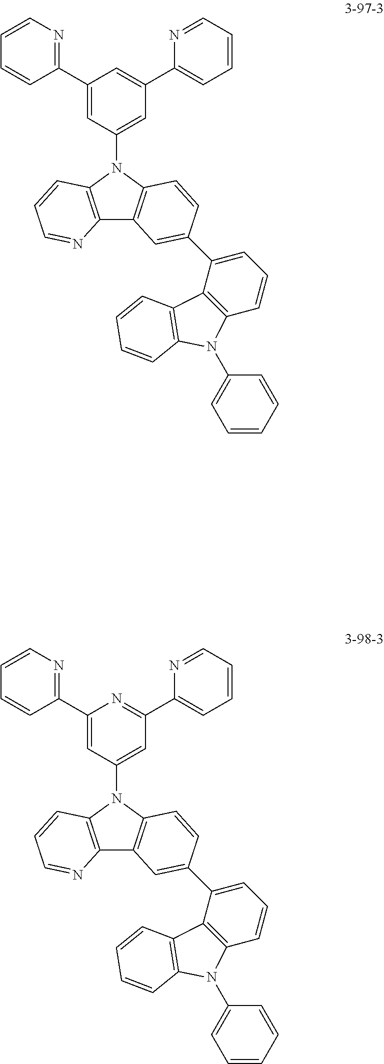 [SMILES: c1ccc(-n2c3ccccc3c3c(-c4ccc5c(c4)c4ncccc4n5-c4cc(-c5ccccn5)cc(-c5ccccn5)c4)cccc32)cc1 ; c1ccc(-n2c3ccccc3c3c(-c4ccc5c(c4)c4ncccc4n5-c4cc(-c5ccccn5)nc(-c5ccccn5)c4)cccc32)cc1]

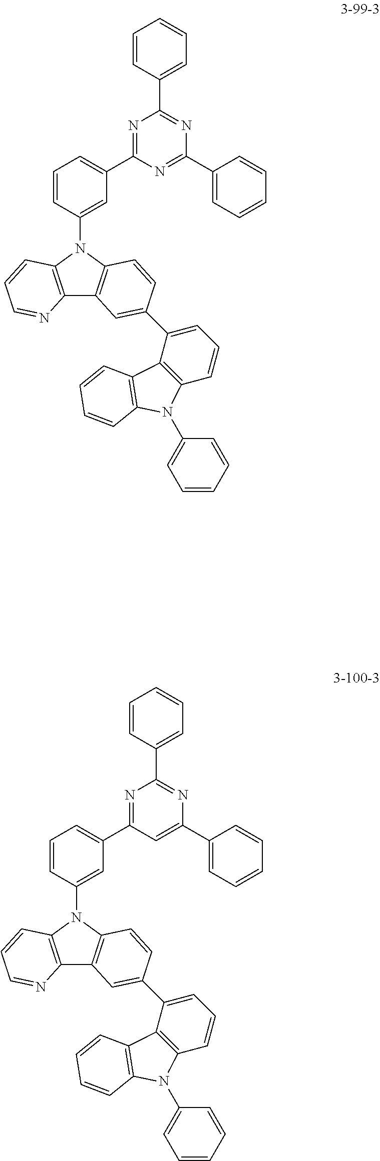 [SMILES: c1ccc(-c2nc(-c3ccccc3)nc(-c3cccc(-n4c5ccc(-c6cccc7c6c6ccccc6n7-c6ccccc6)cc5c5ncccc54)c3)n2)cc1 ; c1ccc(-c2cc(-c3cccc(-n4c5ccc(-c6cccc7c6c6ccccc6n7-c6ccccc6)cc5c5ncccc54)c3)nc(-c3ccccc3)n2)cc1]

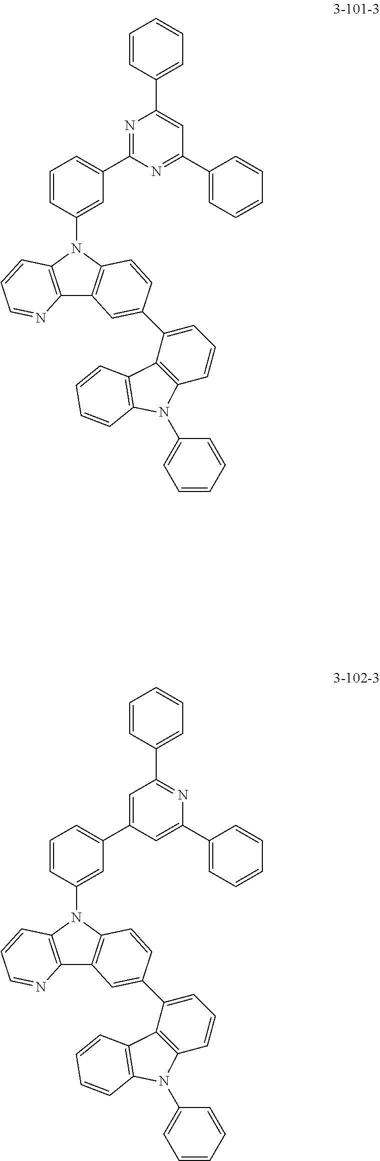 [SMILES: c1ccc(-c2cc(-c3ccccc3)nc(-c3cccc(-n4c5ccc(-c6cccc7c6c6ccccc6n7-c6ccccc6)cc5c5ncccc54)c3)n2)cc1 ; c1ccc(-c2cc(-c3cccc(-n4c5ccc(-c6cccc7c6c6ccccc6n7-c6ccccc6)cc5c5ncccc54)c3)cc(-c3ccccc3)n2)cc1]

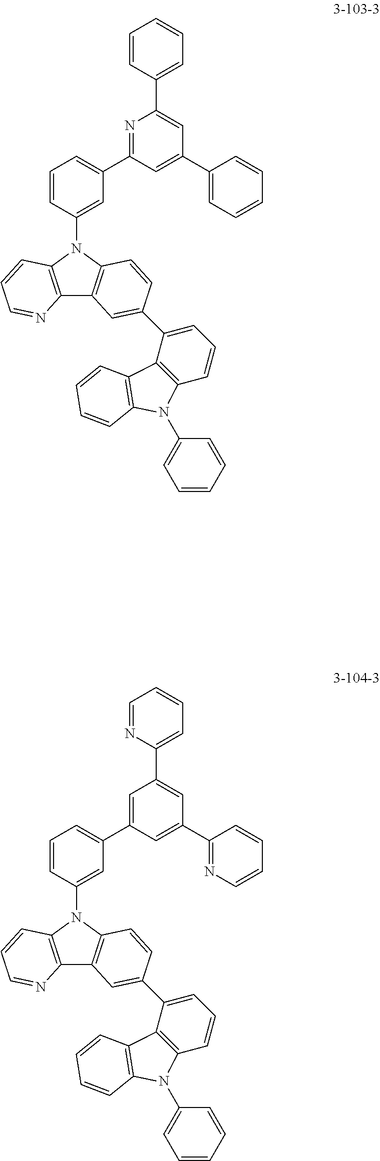 [SMILES: c1ccc(-c2cc(-c3ccccc3)nc(-c3cccc(-n4c5ccc(-c6cccc7c6c6ccccc6n7-c6ccccc6)cc5c5ncccc54)c3)c2)cc1 ; c1ccc(-n2c3ccccc3c3c(-c4ccc5c(c4)c4ncccc4n5-c4cccc(-c5cc(-c6ccccn6)cc(-c6ccccn6)c5)c4)cccc32)cc1]

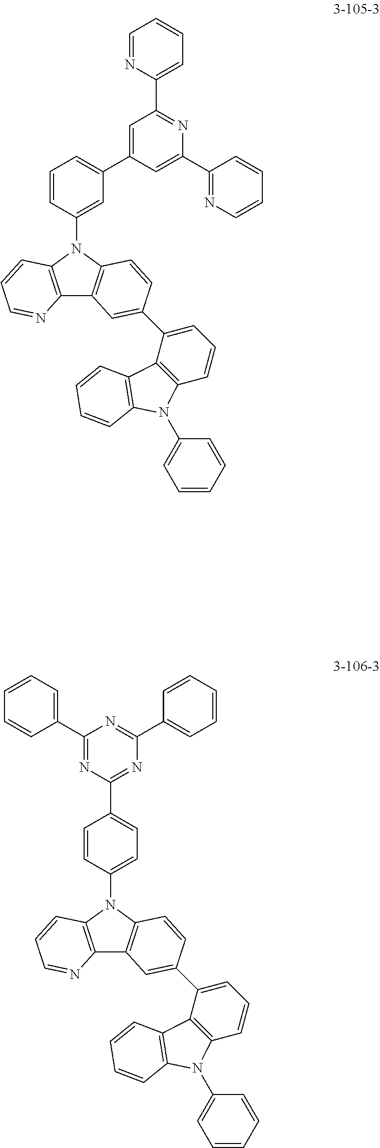 [SMILES: c1ccc(-n2c3ccccc3c3c(-c4ccc5c(c4)c4ncccc4n5-c4cccc(-c5cc(-c6ccccn6)nc(-c6ccccn6)c5)c4)cccc32)cc1 ; c1ccc(-c2nc(-c3ccccc3)nc(-c3ccc(-n4c5ccc(-c6cccc7c6c6ccccc6n7-c6ccccc6)cc5c5ncccc54)cc3)n2)cc1]

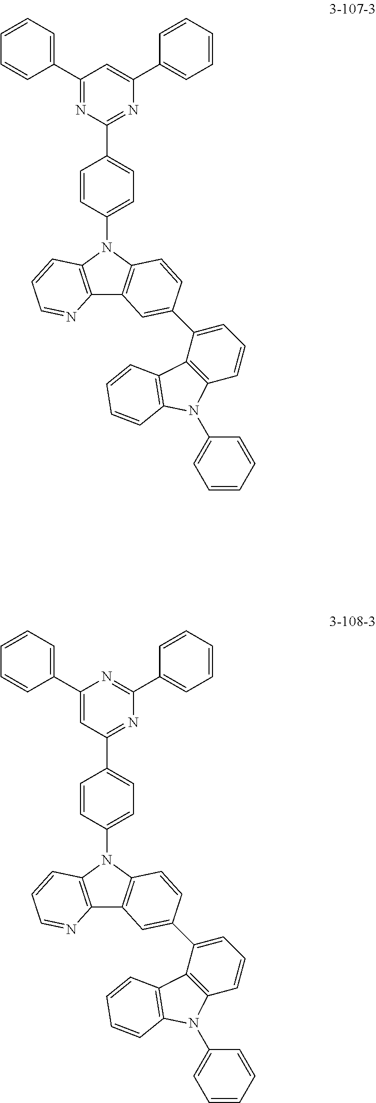 [SMILES: c1ccc(-c2cc(-c3ccccc3)nc(-c3ccc(-n4c5ccc(-c6cccc7c6c6ccccc6n7-c6ccccc6)cc5c5ncccc54)cc3)n2)cc1 ; c1ccc(-c2cc(-c3ccc(-n4c5ccc(-c6cccc7c6c6ccccc6n7-c6ccccc6)cc5c5ncccc54)cc3)nc(-c3ccccc3)n2)cc1]

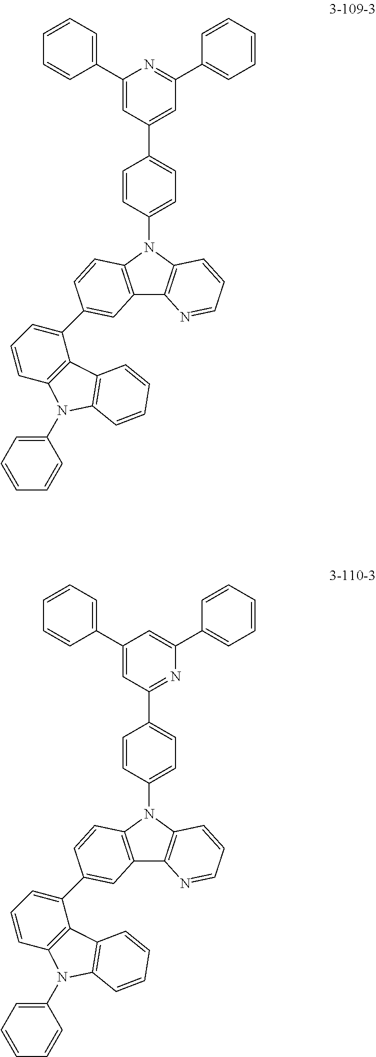 [SMILES: c1ccc(-c2cc(-c3ccc(-n4c5ccc(-c6cccc7c6c6ccccc6n7-c6ccccc6)cc5c5ncccc54)cc3)cc(-c3ccccc3)n2)cc1 ; c1ccc(-c2cc(-c3ccccc3)nc(-c3ccc(-n4c5ccc(-c6cccc7c6c6ccccc6n7-c6ccccc6)cc5c5ncccc54)cc3)c2)cc1]

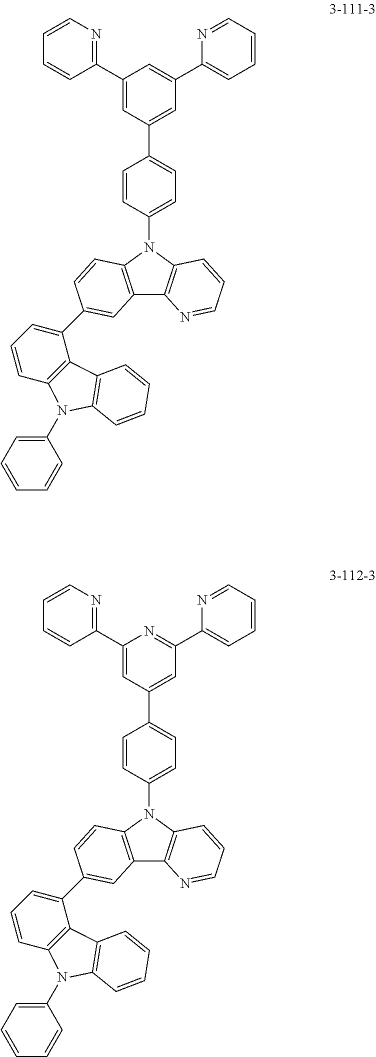 [SMILES: c1ccc(-n2c3ccccc3c3c(-c4ccc5c(c4)c4ncccc4n5-c4ccc(-c5cc(-c6ccccn6)cc(-c6ccccn6)c5)cc4)cccc32)cc1 ; c1ccc(-n2c3ccccc3c3c(-c4ccc5c(c4)c4ncccc4n5-c4ccc(-c5cc(-c6ccccn6)nc(-c6ccccn6)c5)cc4)cccc32)cc1]

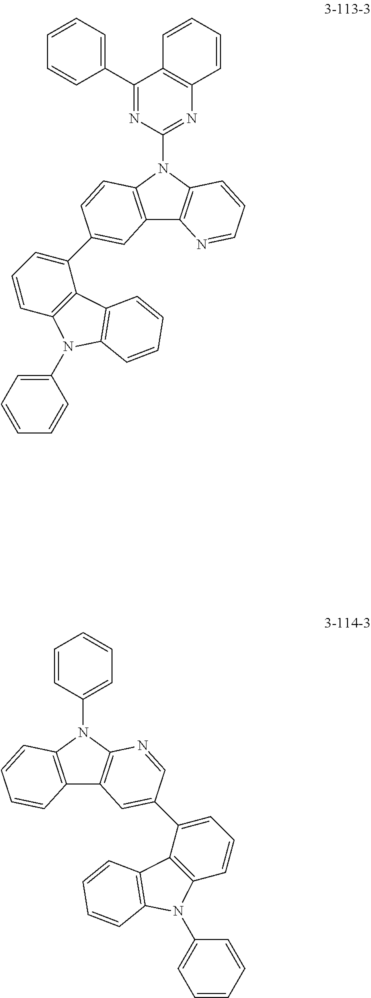 [SMILES: c1ccc(-c2nc(-n3c4ccc(-c5cccc6c5c5ccccc5n6-c5ccccc5)cc4c4ncccc43)nc3ccccc23)cc1 ; c1ccc(-n2c3ccccc3c3c(-c4cnc5c(c4)c4ccccc4n5-c4ccccc4)cccc32)cc1]

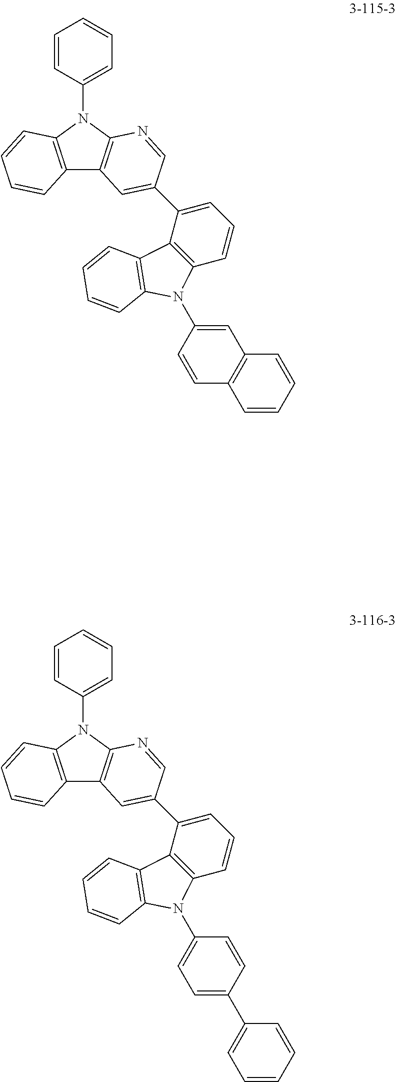 [SMILES: c1ccc(-n2c3ccccc3c3cc(-c4cccc5c4c4ccccc4n5-c4ccc5ccccc5c4)cnc32)cc1 ; c1ccc(-c2ccc(-n3c4ccccc4c4c(-c5cnc6c(c5)c5ccccc5n6-c5ccccc5)cccc43)cc2)cc1]

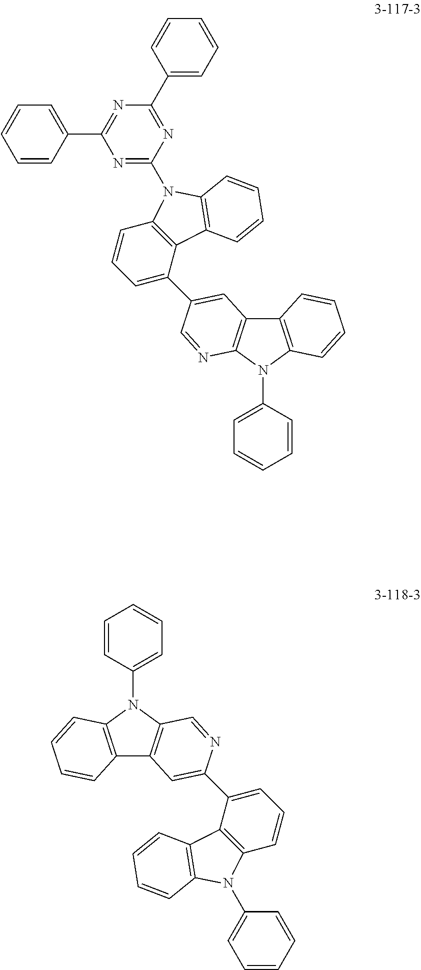 [SMILES: c1ccc(-c2nc(-c3ccccc3)nc(-n3c4ccccc4c4c(-c5cnc6c(c5)c5ccccc5n6-c5ccccc5)cccc43)n2)cc1 ; c1ccc(-n2c3ccccc3c3cc(-c4cccc5c4c4ccccc4n5-c4ccccc4)ncc32)cc1]

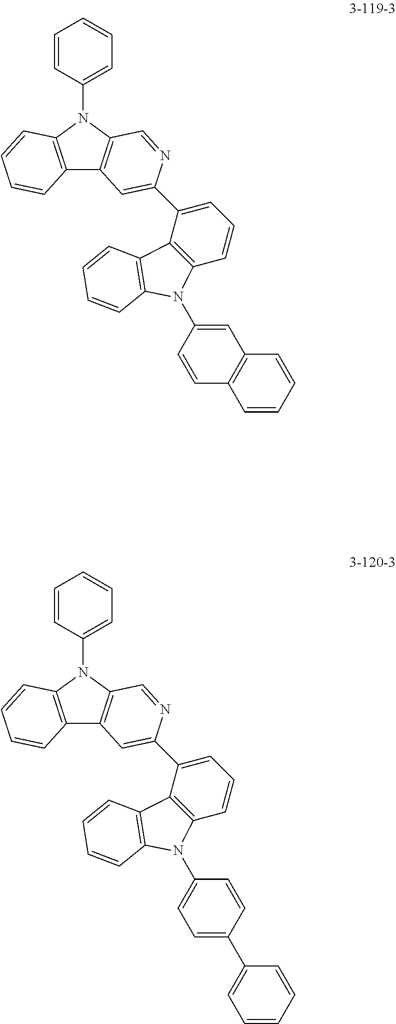 [SMILES: c1ccc(-n2c3ccccc3c3cc(-c4cccc5c4c4ccccc4n5-c4ccc5ccccc5c4)ncc32)cc1 ; c1ccc(-c2ccc(-n3c4ccccc4c4c(-c5cc6c7ccccc7n(-c7ccccc7)c6cn5)cccc43)cc2)cc1]

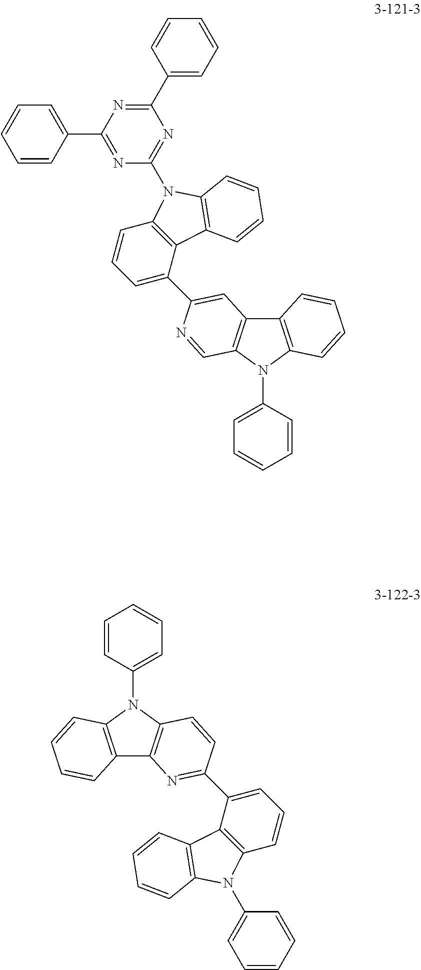 [SMILES: c1ccc(-c2nc(-c3ccccc3)nc(-n3c4ccccc4c4c(-c5cc6c7ccccc7n(-c7ccccc7)c6cn5)cccc43)n2)cc1 ; c1ccc(-n2c3ccccc3c3nc(-c4cccc5c4c4ccccc4n5-c4ccccc4)ccc32)cc1]

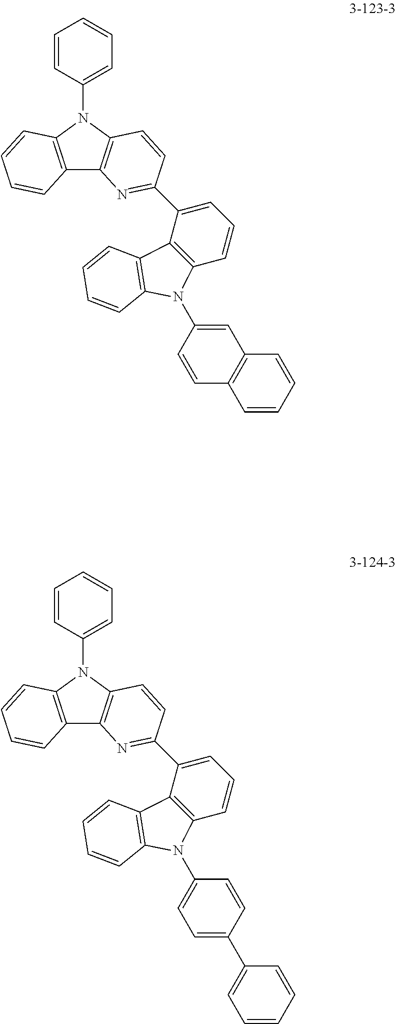 [SMILES: c1ccc(-n2c3ccccc3c3nc(-c4cccc5c4c4ccccc4n5-c4ccc5ccccc5c4)ccc32)cc1 ; c1ccc(-c2ccc(-n3c4ccccc4c4c(-c5ccc6c(n5)c5ccccc5n6-c5ccccc5)cccc43)cc2)cc1]

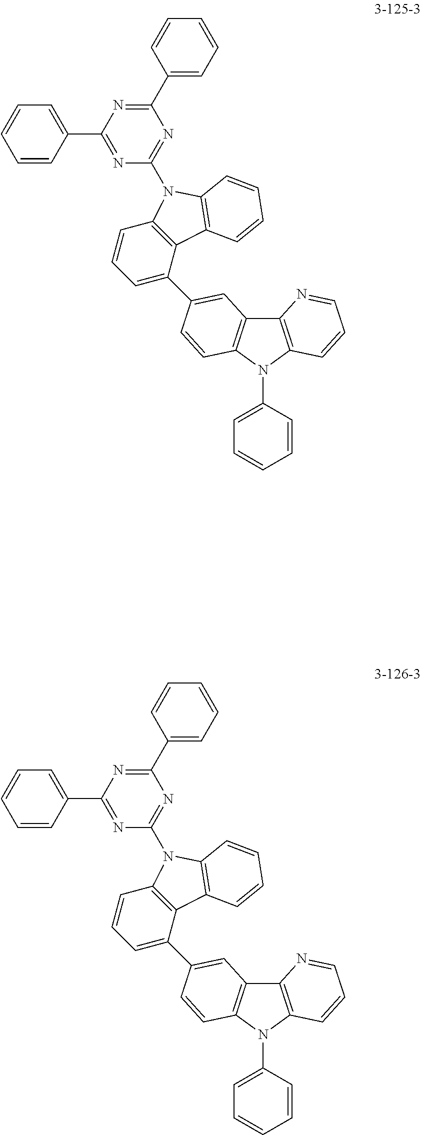 [SMILES: c1ccc(-c2nc(-c3ccccc3)nc(-n3c4ccccc4c4c(-c5ccc6c(c5)c5ncccc5n6-c5ccccc5)cccc43)n2)cc1 ; c1ccc(-c2nc(-c3ccccc3)nc(-n3c4ccccc4c4c(-c5ccc6c(c5)c5ncccc5n6-c5ccccc5)cccc43)n2)cc1]

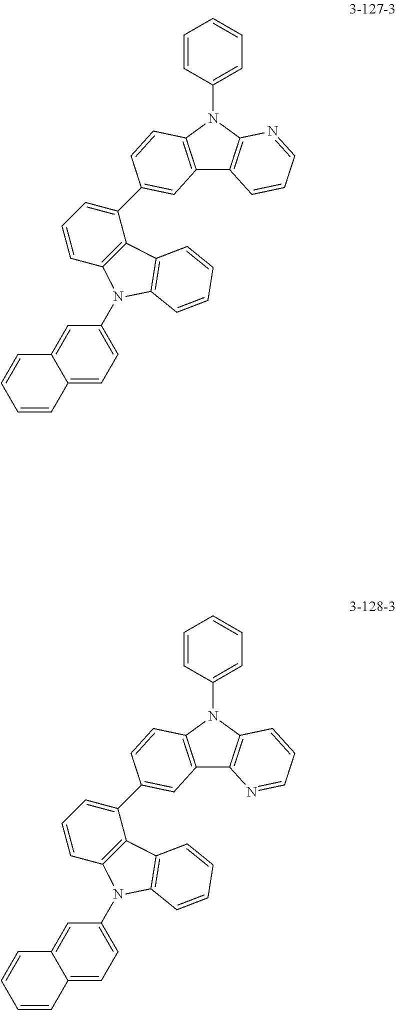 [SMILES: c1ccc(-n2c3ccc(-c4cccc5c4c4ccccc4n5-c4ccc5ccccc5c4)cc3c3cccnc32)cc1 ; c1ccc(-n2c3ccc(-c4cccc5c4c4ccccc4n5-c4ccc5ccccc5c4)cc3c3ncccc32)cc1]

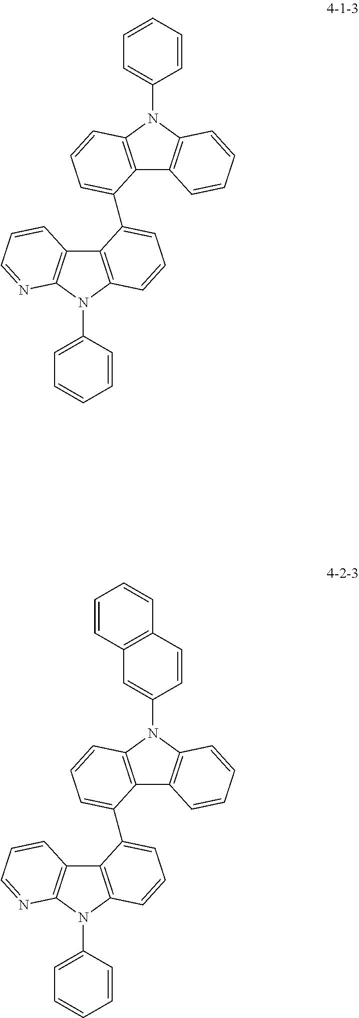 [SMILES: c1ccc(-n2c3ccccc3c3c(-c4cccc5c4c4cccnc4n5-c4ccccc4)cccc32)cc1 ; c1ccc(-n2c3cccc(-c4cccc5c4c4ccccc4n5-c4ccc5ccccc5c4)c3c3cccnc32)cc1]

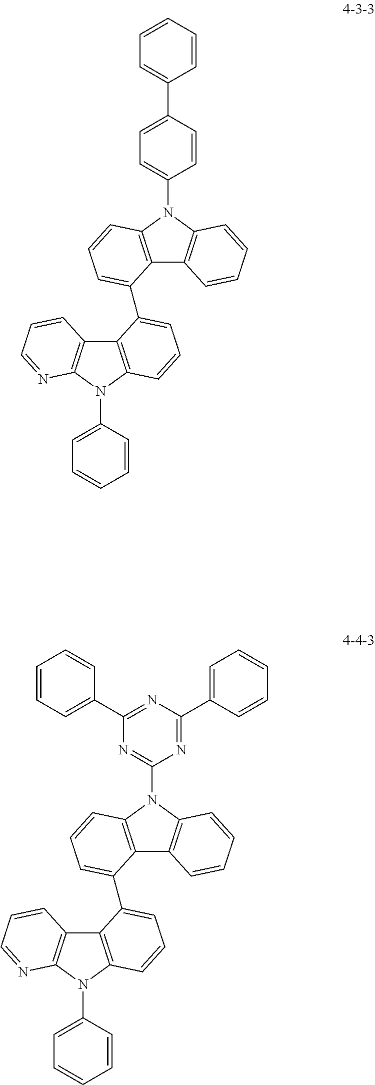 [SMILES: c1ccc(-c2ccc(-n3c4ccccc4c4c(-c5cccc6c5c5cccnc5n6-c5ccccc5)cccc43)cc2)cc1 ; c1ccc(-c2nc(-c3ccccc3)nc(-n3c4ccccc4c4c(-c5cccc6c5c5cccnc5n6-c5ccccc5)cccc43)n2)cc1]

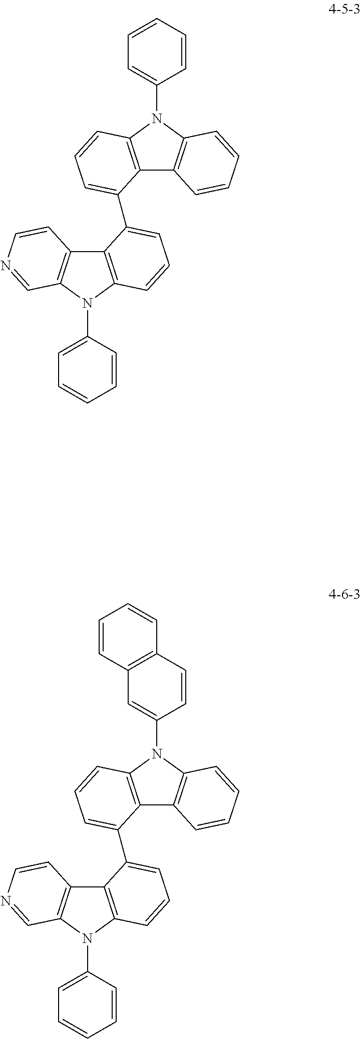 [SMILES: c1ccc(-n2c3ccccc3c3c(-c4cccc5c4c4ccncc4n5-c4ccccc4)cccc32)cc1 ; c1ccc(-n2c3cnccc3c3c(-c4cccc5c4c4ccccc4n5-c4ccc5ccccc5c4)cccc32)cc1]

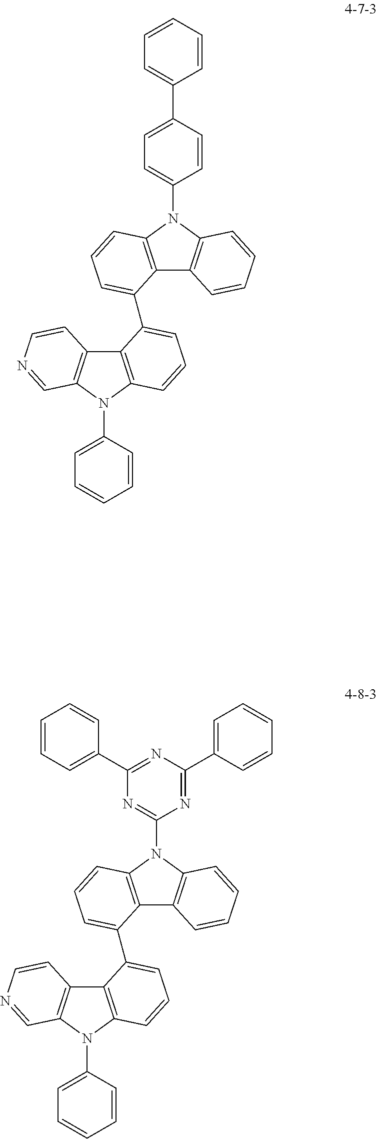 [SMILES: c1ccc(-c2ccc(-n3c4ccccc4c4c(-c5cccc6c5c5ccncc5n6-c5ccccc5)cccc43)cc2)cc1 ; c1ccc(-c2nc(-c3ccccc3)nc(-n3c4ccccc4c4c(-c5cccc6c5c5ccncc5n6-c5ccccc5)cccc43)n2)cc1]

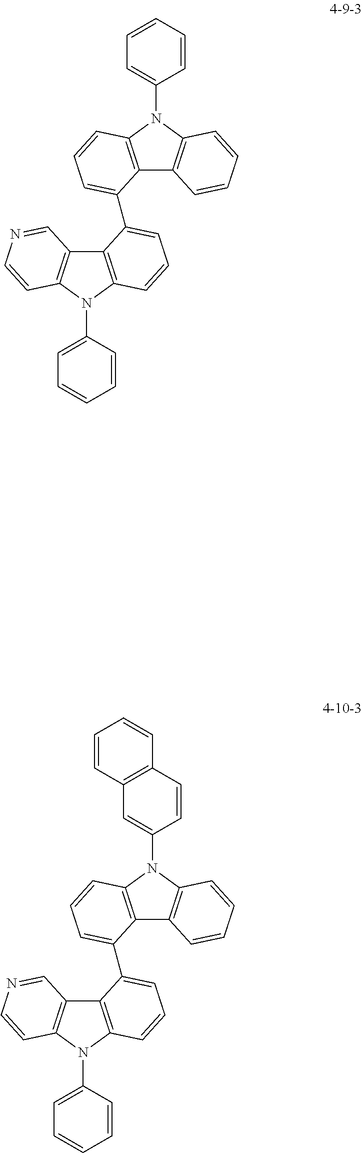 [SMILES: c1ccc(-n2c3ccccc3c3c(-c4cccc5c4c4cnccc4n5-c4ccccc4)cccc32)cc1 ; c1ccc(-n2c3ccncc3c3c(-c4cccc5c4c4ccccc4n5-c4ccc5ccccc5c4)cccc32)cc1]

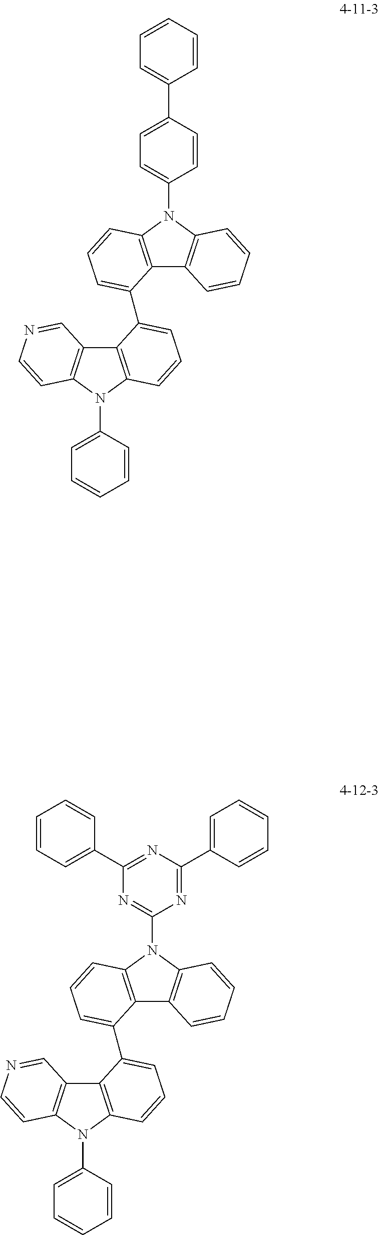 [SMILES: c1ccc(-c2ccc(-n3c4ccccc4c4c(-c5cccc6c5c5cnccc5n6-c5ccccc5)cccc43)cc2)cc1 ; c1ccc(-c2nc(-c3ccccc3)nc(-n3c4ccccc4c4c(-c5cccc6c5c5cnccc5n6-c5ccccc5)cccc43)n2)cc1]

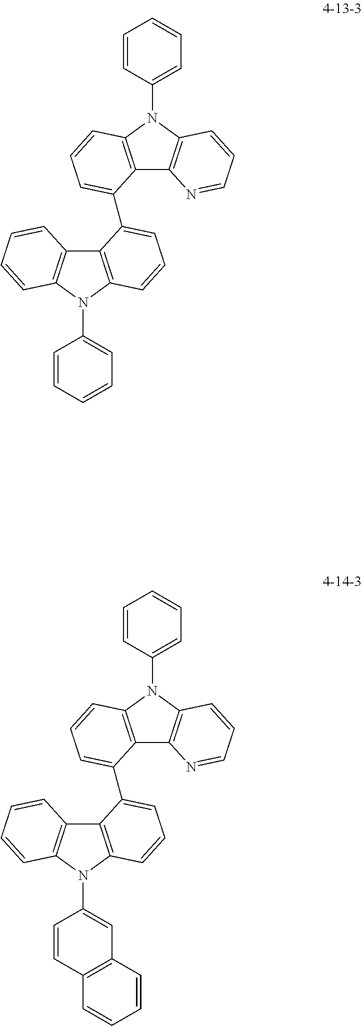 [SMILES: c1ccc(-n2c3ccccc3c3c(-c4cccc5c4c4ncccc4n5-c4ccccc4)cccc32)cc1 ; c1ccc(-n2c3cccnc3c3c(-c4cccc5c4c4ccccc4n5-c4ccc5ccccc5c4)cccc32)cc1]

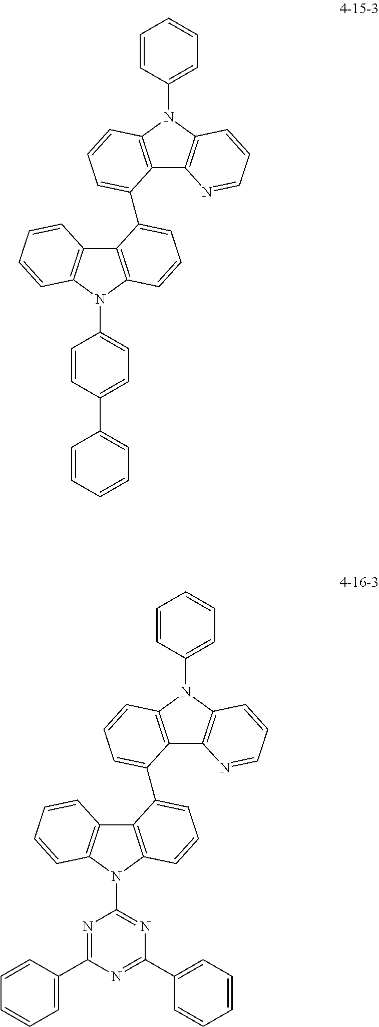 [SMILES: c1ccc(-c2ccc(-n3c4ccccc4c4c(-c5cccc6c5c5ncccc5n6-c5ccccc5)cccc43)cc2)cc1 ; c1ccc(-c2nc(-c3ccccc3)nc(-n3c4ccccc4c4c(-c5cccc6c5c5ncccc5n6-c5ccccc5)cccc43)n2)cc1]

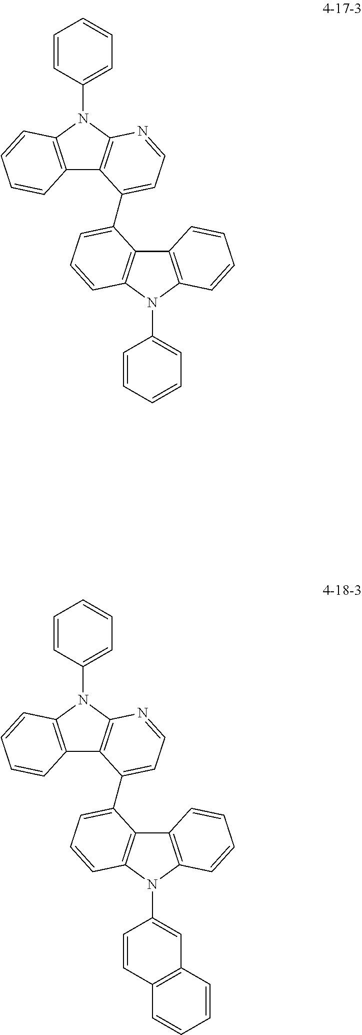 [SMILES: c1ccc(-n2c3ccccc3c3c(-c4ccnc5c4c4ccccc4n5-c4ccccc4)cccc32)cc1 ; c1ccc(-n2c3ccccc3c3c(-c4cccc5c4c4ccccc4n5-c4ccc5ccccc5c4)ccnc32)cc1]

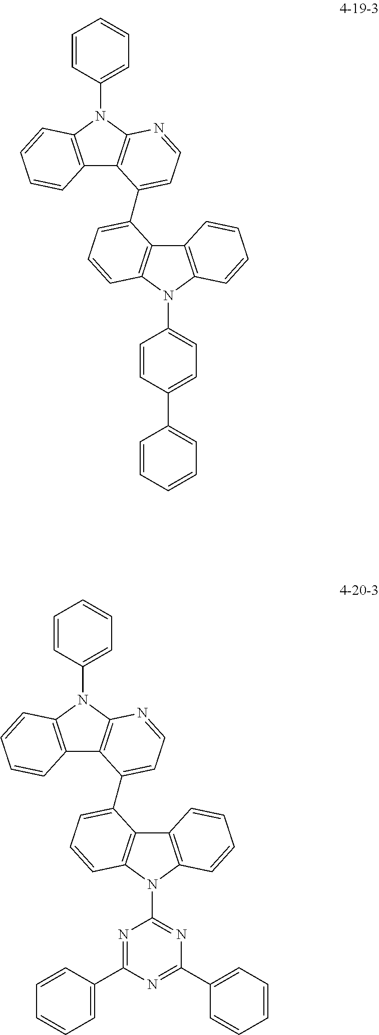 [SMILES: c1ccc(-c2ccc(-n3c4ccccc4c4c(-c5ccnc6c5c5ccccc5n6-c5ccccc5)cccc43)cc2)cc1 ; c1ccc(-c2nc(-c3ccccc3)nc(-n3c4ccccc4c4c(-c5ccnc6c5c5ccccc5n6-c5ccccc5)cccc43)n2)cc1]

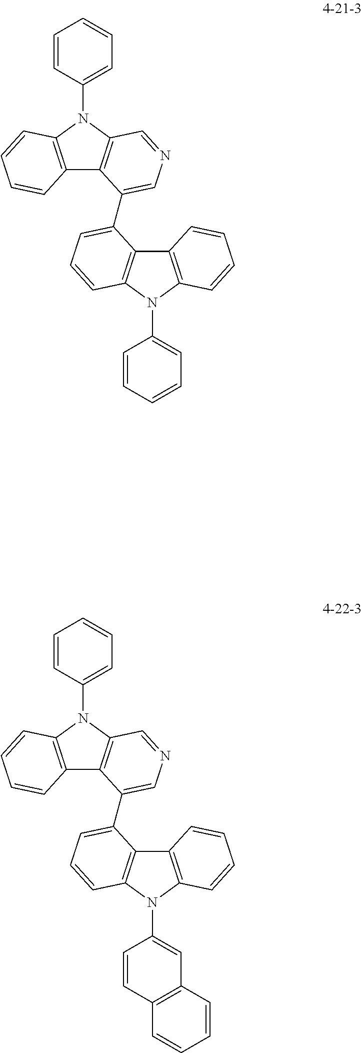 [SMILES: c1ccc(-n2c3ccccc3c3c(-c4cncc5c4c4ccccc4n5-c4ccccc4)cccc32)cc1 ; c1ccc(-n2c3ccccc3c3c(-c4cccc5c4c4ccccc4n5-c4ccc5ccccc5c4)cncc32)cc1]

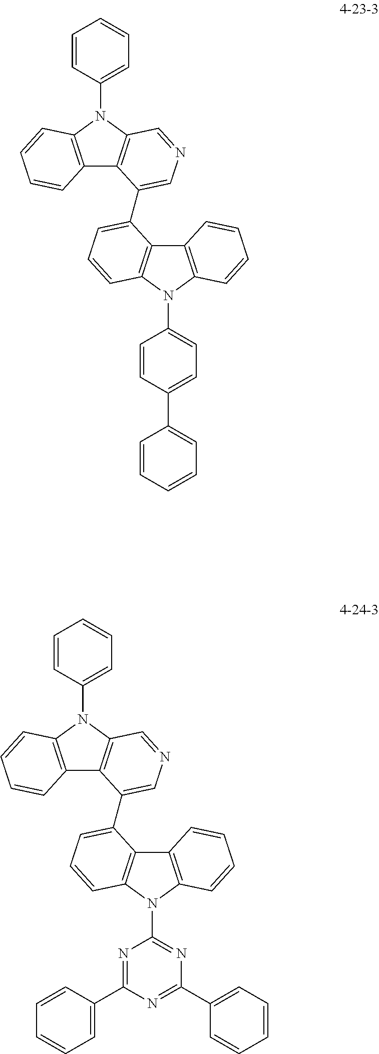 [SMILES: c1ccc(-c2ccc(-n3c4ccccc4c4c(-c5cncc6c5c5ccccc5n6-c5ccccc5)cccc43)cc2)cc1 ; c1ccc(-c2nc(-c3ccccc3)nc(-n3c4ccccc4c4c(-c5cncc6c5c5ccccc5n6-c5ccccc5)cccc43)n2)cc1]

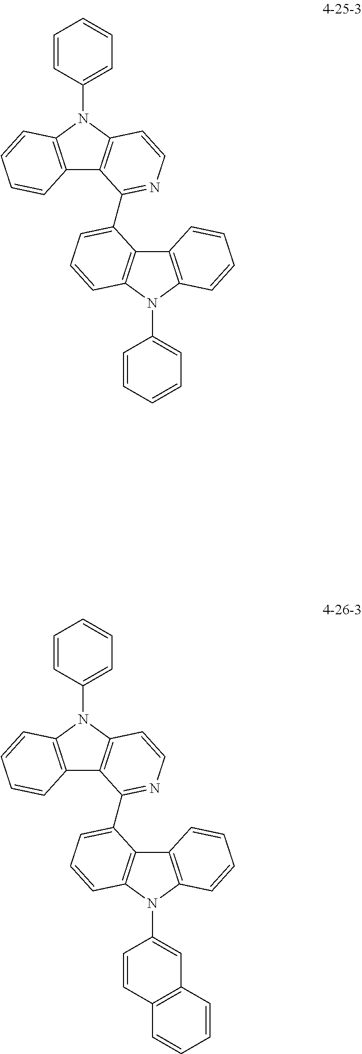 [SMILES: c1ccc(-n2c3ccccc3c3c(-c4nccc5c4c4ccccc4n5-c4ccccc4)cccc32)cc1 ; c1ccc(-n2c3ccccc3c3c(-c4cccc5c4c4ccccc4n5-c4ccc5ccccc5c4)nccc32)cc1]

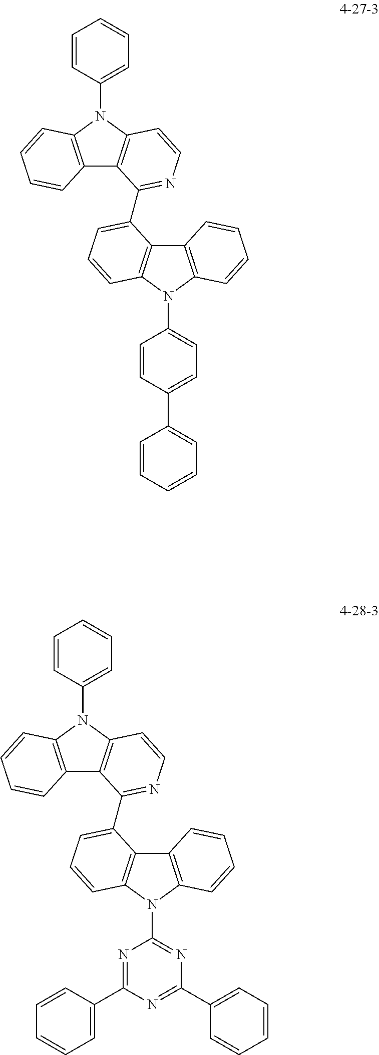 [SMILES: c1ccc(-c2ccc(-n3c4ccccc4c4c(-c5nccc6c5c5ccccc5n6-c5ccccc5)cccc43)cc2)cc1 ; c1ccc(-c2nc(-c3ccccc3)nc(-n3c4ccccc4c4c(-c5nccc6c5c5ccccc5n6-c5ccccc5)cccc43)n2)cc1]

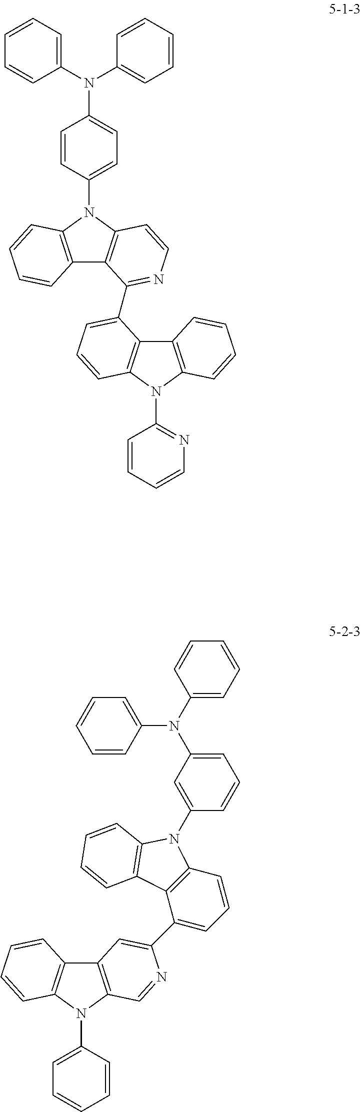 [SMILES: c1ccc(N(c2ccccc2)c2ccc(-n3c4ccccc4c4c(-c5cccc6c5c5ccccc5n6-c5ccccn5)nccc43)cc2)cc1 ; c1ccc(N(c2ccccc2)c2cccc(-n3c4ccccc4c4c(-c5cc6c7ccccc7n(-c7ccccc7)c6cn5)cccc43)c2)cc1]

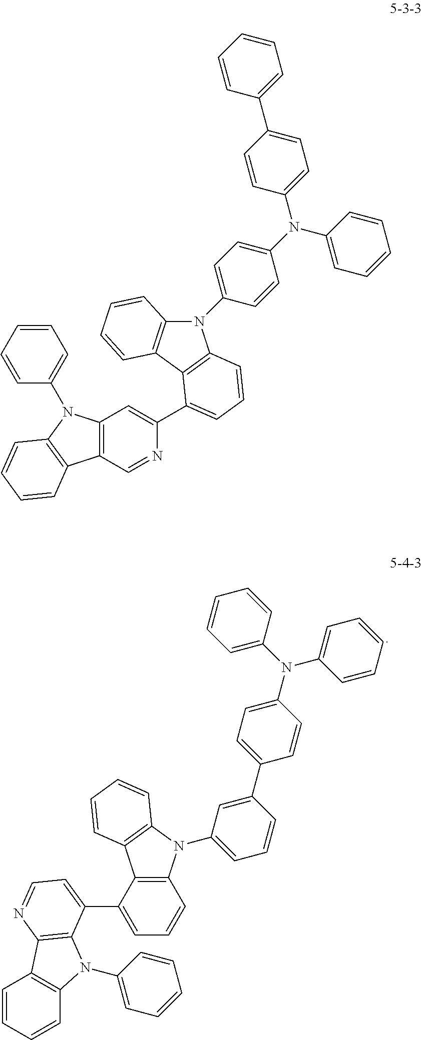 [SMILES: c1ccc(-c2ccc(N(c3ccccc3)c3ccc(-n4c5ccccc5c5c(-c6cc7c(cn6)c6ccccc6n7-c6ccccc6)cccc54)cc3)cc2)cc1 ; c1ccc(N(c2ccccc2)c2ccc(-c3cccc(-n4c5ccccc5c5c(-c6ccnc7c8ccccc8n(-c8ccccc8)c67)cccc54)c3)cc2)cc1]

In another embodiment, the present invention provides a compound for an organic electronic element, represented by Formula 3-1.

In still another embodiment, the present invention provides an organic electronic element containing the compound represented by Formula 3-1.

Here, the organic electronic element may include: a first electrode; a second electrode; and an organic material layer positioned between the first electrode and the second electrode, wherein the organic material layer may contain a compound represented by Formula 3-1, and the compound represented by Formula 3-1 may be contained in at least one of a hole injection layer, a hole transport layer, an auxiliary light emitting layer, a light emitting layer, an electron transport layer, and an electron injection layer for an organic material layer. Especially, the compound represented by Formula 3-1 may be contained in the light emitting layer.

That is, the compound represented by Formula 3-1 may be used as a material for a hole injection layer, a hole transport layer, an auxiliary light emitting layer, a light emitting layer, an electron transport layer, or an electron injection layer. Especially, the compound represented by Formula 3-1 may be used as a material for the light emitting layer. The present invention provides, specifically, an organic electronic element including the organic material layer containing one of the compounds represented by Formulas 3-2 to 3-13, and more specifically, an organic electronic element including the organic material layer containing the compound represented by an individual formula (1-1-3 to 1-28-3, 2-1-3 to 2-128-3, 3-1-3 to 3-128-3, 4-1-3 to 4-28-3, and 5-1-3 to 5-4-3).

In still another embodiment, the present invention provides an organic electronic element, in which the compound is contained alone, two or more different types of the compounds are contained as a combination, or the compound is contained together with other compounds as a combination of two or more in at least one of the hole injection layer, the hole transport layer, the auxiliary light emitting layer, the light emitting layer, the electron transport layer, and the electron injection layer of the organic material layer. In other words, the compounds corresponding to Formulas 3-1 to 3-13 may be contained alone, a mixture of two or more kinds of compounds of Formulas 3-1 to 3-13 may be contained, or a mixture of the compound of claims and a compound not corresponding to the present invention may be contained in each of the layers. Here, the compounds that do not correspond to the present invention may be a single compound or two or more kinds of compounds.

Here, when the compound is contained together with other compounds as a combination of two or more kinds of compounds, another compound may be a compound that is already known for each organic material layer, or a compound to be developed in the future. Here, the compounds contained in the organic material layer may be composed of only the same kind of compounds, or a mixture of two or more kinds of different compounds represented by formula 3-1.

In still another embodiment of the present invention, the present invention provides an organic electronic element further including a light efficiency improvement layer, which is formed on at least one of one side of one surface of the first electrode, which is opposite to the organic material layer and one side of one surface of the second electrode, which is opposite to the organic material layer.

Hereinafter, synthesis examples of the compound represented by Formula 3-1 and manufacturing examples of the organic electronic element according to the present invention will be described in detail by way of example. However, the following examples are only for illustrative purposes and are not intended to limit the scope of the invention.

### SYNTHESIS EXAMPLES

The product represented by Formula 3-1 according to the present invention is prepared by reaction of Sub 1-3 and Sub 2-3 as in Reaction Scheme 3-1 below, but are not limited thereto.

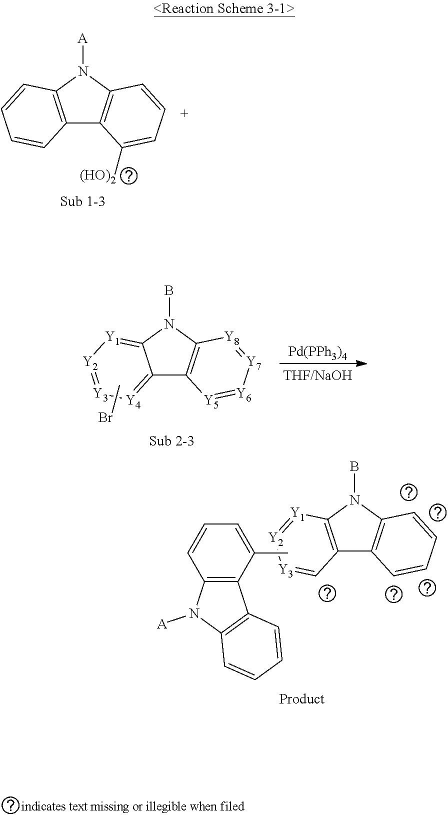

### □. Synthesis Example of Sub 1-3

Sub 1-3 in Reaction Scheme 3-1 may be synthesized via the reaction pathway of Reaction Scheme 3-2 below, but is not limited thereto.

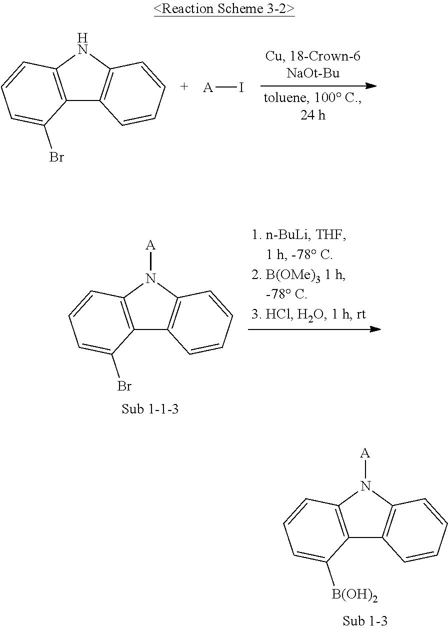

### Synthesis Sub 1-1-3

After bromo-9H-carbazole (203 mmol) and an iodo compound (240 mmol) were mixed with 800 mL of toluene, Cu (764 mg, 12 mmol), 18-Crown-6 (6.3 g, 24 mmol), and NaOt-Bu (57.6 g, 600 mmol) were added thereto, and the mixture was stirred under reflux at 100□ for 24 h. After extraction with ether and water, the organic layer was dried over MgSO₄ and concentrated, and then the generated organic material was subjected to silica gel column chromatography and recrystallization to give an intermediate.

### Synthesis of Sub 1-1(1)-3

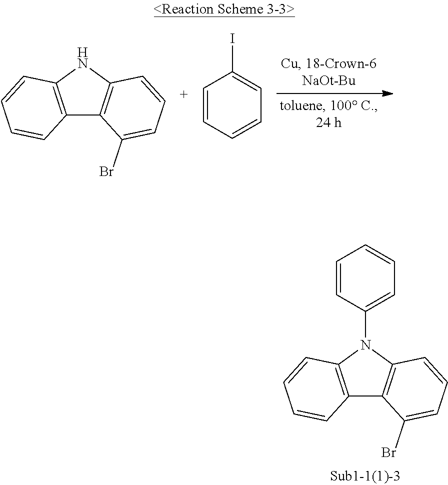

After bromo-9H-carbazole (50 g, 203 mmol) and iodobenzene (49 g, 240 mmol) were mixed with 800 mL of toluene, Cu (764 mg, 12 mmol), 18-Crown-6 (6.3 g, 24 mmol), and NaOt-Bu (57.6 g, 600 mmol) were added thereto, and the mixture was stirred under reflux at 100□ for 24 h. After extraction with ether and water, the organic layer was dried over MgSO₄ and concentrated, and then the generated organic material was subjected to silica gel column chromatography and recrystallization to give 36.6 g of Sub 1-1(1)-3 (yield: 57%).

Examples of Sub 1-1-3 are as follows, but are limited thereto, and FD-MS values thereof are shown in table 3-1

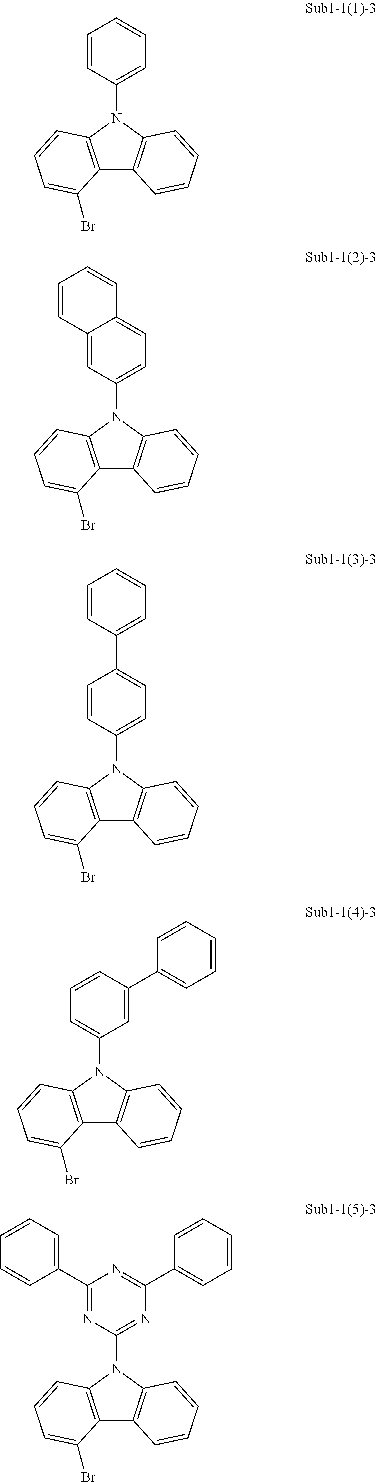

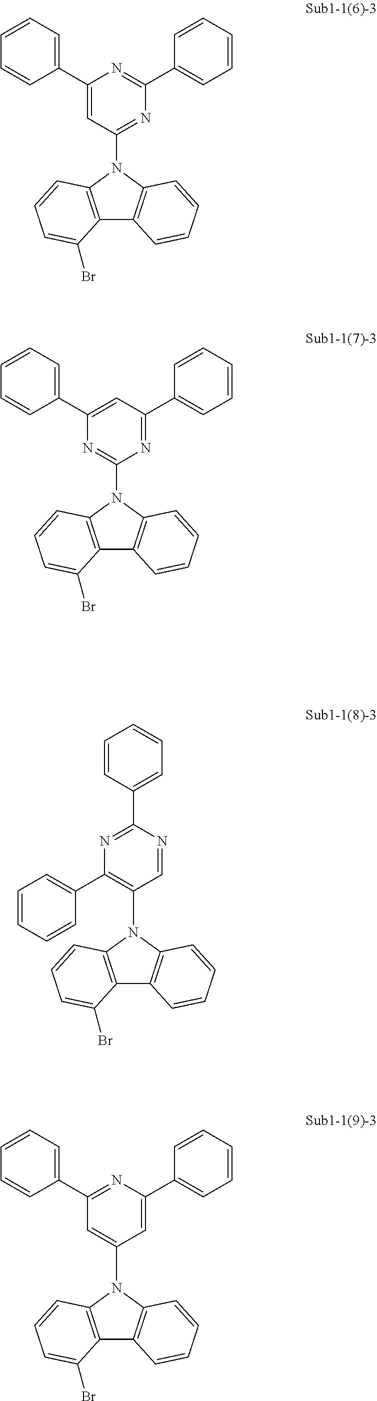 [SMILES: Brc1cccc2c1c1ccccc1n2-c1cc(-c2ccccc2)nc(-c2ccccc2)n1 ; Brc1cccc2c1c1ccccc1n2-c1nc(-c2ccccc2)cc(-c2ccccc2)n1 ; Brc1cccc2c1c1ccccc1n2-c1cnc(-c2ccccc2)nc1-c1ccccc1 ; Brc1cccc2c1c1ccccc1n2-c1cc(-c2ccccc2)nc(-c2ccccc2)c1]

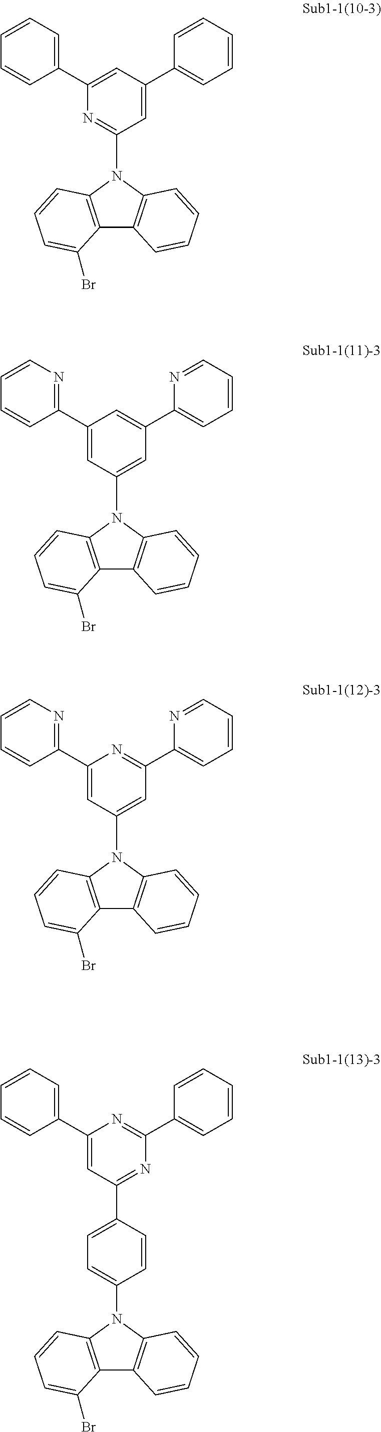 [SMILES: Brc1cccc2c1c1ccccc1n2-c1cc(-c2ccccc2)cc(-c2ccccc2)n1 ; Brc1cccc2c1c1ccccc1n2-c1cc(-c2ccccn2)cc(-c2ccccn2)c1 ; Brc1cccc2c1c1ccccc1n2-c1cc(-c2ccccn2)nc(-c2ccccn2)c1 ; Brc1cccc2c1c1ccccc1n2-c1ccc(-c2cc(-c3ccccc3)nc(-c3ccccc3)n2)cc1]

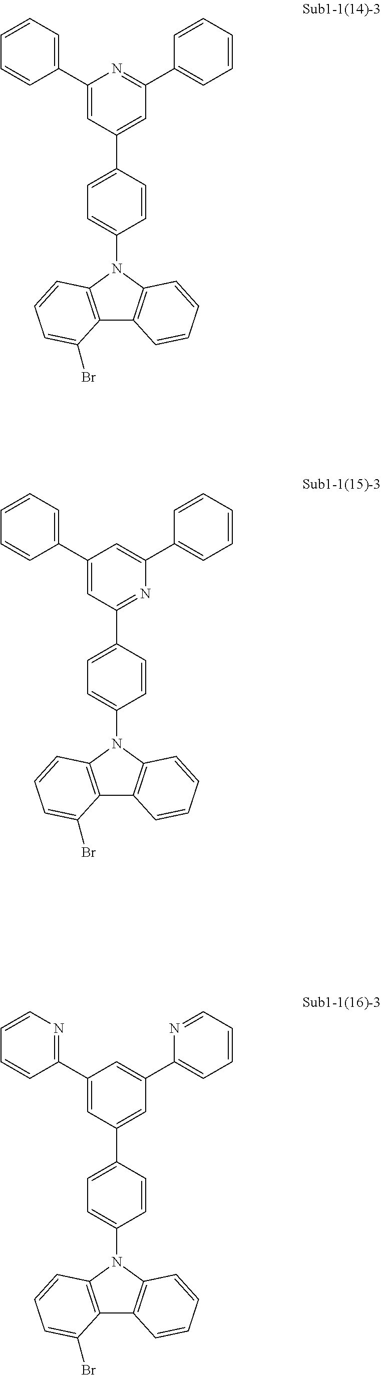 [SMILES: Brc1cccc2c1c1ccccc1n2-c1ccc(-c2cc(-c3ccccc3)nc(-c3ccccc3)c2)cc1 ; Brc1cccc2c1c1ccccc1n2-c1ccc(-c2cc(-c3ccccc3)cc(-c3ccccc3)n2)cc1 ; Brc1cccc2c1c1ccccc1n2-c1ccc(-c2cc(-c3ccccn3)cc(-c3ccccn3)c2)cc1]

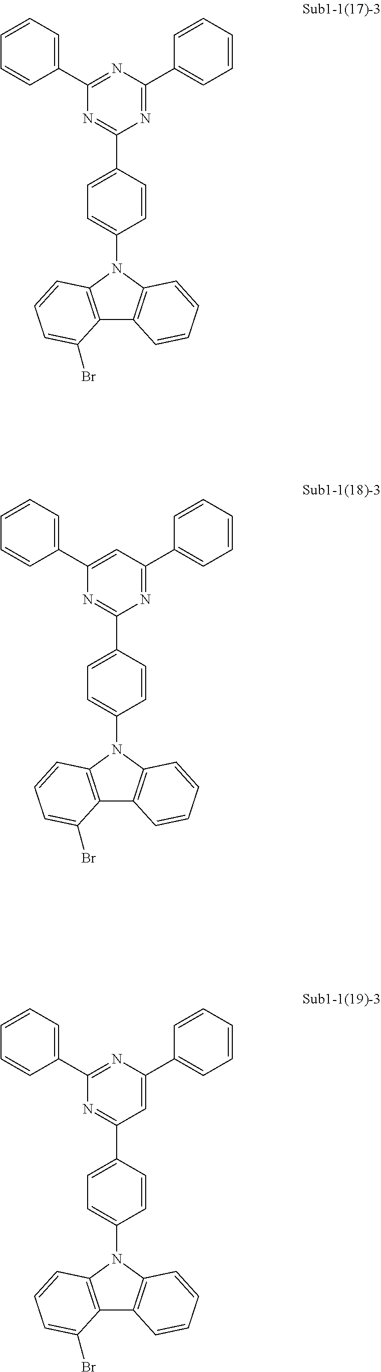 [SMILES: Brc1cccc2c1c1ccccc1n2-c1ccc(-c2nc(-c3ccccc3)nc(-c3ccccc3)n2)cc1 ; Brc1cccc2c1c1ccccc1n2-c1ccc(-c2nc(-c3ccccc3)cc(-c3ccccc3)n2)cc1 ; Brc1cccc2c1c1ccccc1n2-c1ccc(-c2cc(-c3ccccc3)nc(-c3ccccc3)n2)cc1]

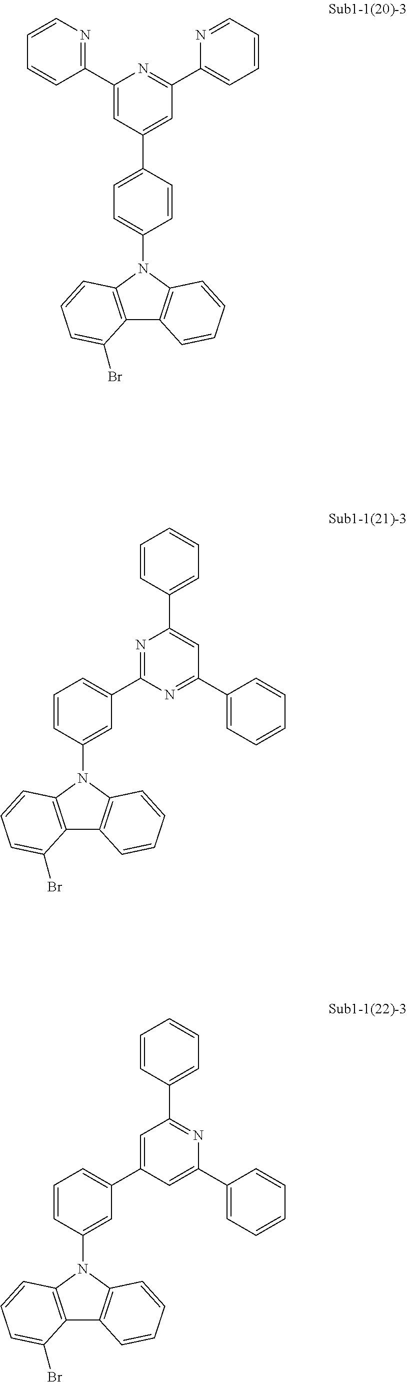 [SMILES: Brc1cccc2c1c1ccccc1n2-c1ccc(-c2cc(-c3ccccn3)nc(-c3ccccn3)c2)cc1 ; Brc1cccc2c1c1ccccc1n2-c1cccc(-c2nc(-c3ccccc3)cc(-c3ccccc3)n2)c1 ; Brc1cccc2c1c1ccccc1n2-c1cccc(-c2cc(-c3ccccc3)nc(-c3ccccc3)c2)c1]

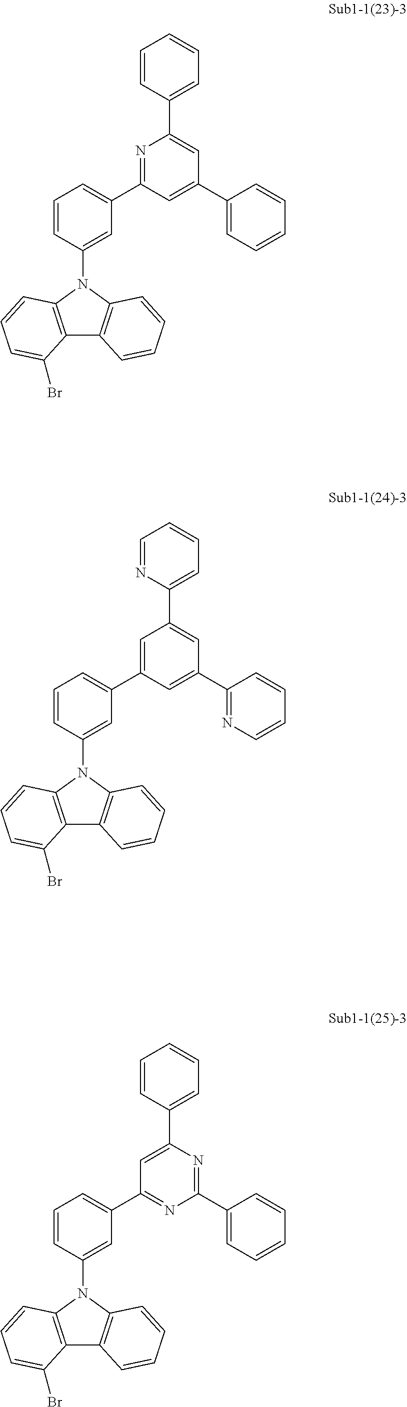 [SMILES: Brc1cccc2c1c1ccccc1n2-c1cccc(-c2cc(-c3ccccc3)cc(-c3ccccc3)n2)c1 ; Brc1cccc2c1c1ccccc1n2-c1cccc(-c2cc(-c3ccccn3)cc(-c3ccccn3)c2)c1 ; Brc1cccc2c1c1ccccc1n2-c1cccc(-c2cc(-c3ccccc3)nc(-c3ccccc3)n2)c1]

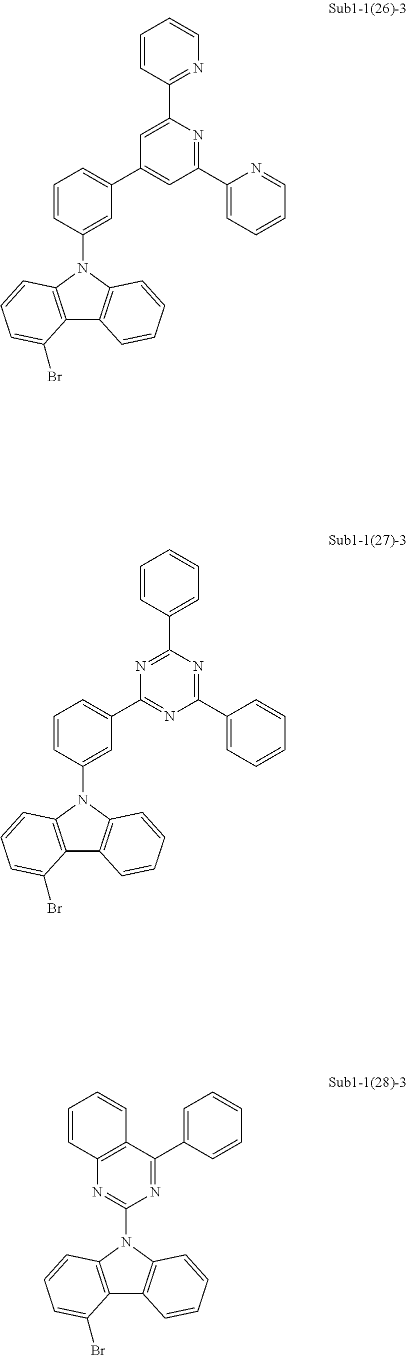 [SMILES: Brc1cccc2c1c1ccccc1n2-c1cccc(-c2cc(-c3ccccn3)nc(-c3ccccn3)c2)c1 ; Brc1cccc2c1c1ccccc1n2-c1cccc(-c2nc(-c3ccccc3)nc(-c3ccccc3)n2)c1 ; Brc1cccc2c1c1ccccc1n2-c1nc(-c2ccccc2)c2ccccc2n1]

TABLE 3-1

| Compound | FD-MS | Compound | FD-MS |
|---|---|---|---|
| Sub1-1(1)-3 | m/z = 321.02(C₁₈H₁₂BrN = 322.20) | Sub1-1(2)-3 | m/z = 371.03(C₂₂H₁₄BrN = 372.26) |
| Sub1-1(3)-3 | m/z = 397.05(C₂₄H₁₆BrN = 398.29) | Sub1-1(4)-3 | m/z = 397.05(C₂₄H₁₆BrN = 398.29) |
| Sub1-1(5)-3 | m/z = 476.06(C₂₂H₁₇BrN₄ = 477.35) | Sub1-1(6)-3 | m/z = 475.07(C₂₈H₁₈BrN₃ = 476.37) |
| Sub1-1(7)-3 | m/z = 475.07(C₂₈H₁₈BrN₃ = 476.37) | Sub1-1(8)-3 | m/z = 475.07(C₂₈H₁₈BrN₃ = 476.37) |
| Sub1-1(9)-3 | m/z = 474.07(C₂₉H₁₉BrN₂ = 475.38) | Sub1-1(10)-3 | m/z = 474.07(C₂₉H₁₉BrN₂ = 475.38) |
| Sub1-1(11)-3 | m/z = 475.07(C₂₈H₁₉BrN₃ = 476.37 | Sub1-1(12)-3 | m/z = 476.06(C₂₇H₁₇BrN₄ = 477.35) |
| Sub1-1(13)-3 | m/z = 551.10(C₃₄H₂₂BrN₃ = 552.46) | Sub1-1(14)-3 | m/z = 550.10(C₃₅H₂₃BrN₂ = 551.47) |
| Sub1-1(15)-3 | m/z = 550.10(C₃₅H₂₃BrN₂ = 551.47 | Sub1-1(16)-3 | m/z = 551.10(C₃₄H₂₂BrN₃ = 552.46) |
| Sub1-1(17)-3 | m/z = 552.09(C₃₂H₂₁BrN₄ = 553.45) | Sub1-1(18)-3 | m/z = 551.10(C₃₄H₂₂BrN₃ = 552.46) |
| Sub1-1(19)-3 | m/z = 551.10(C₃₄H₂₂BrN₃ = 552.46) | Sub1-1(20)-3 | m/z = 552.09(C₃₃H₂₁BrN₄ = 553.45) |
| Sub1-1(21)-3 | m/z = 551.10(C₃₄H₂₂BrN₃ = 552.46) | Sub1-1(22)-3 | m/z = 550.10(C₃₅H₂₃BrN₂ = 551.47) |
| Sub1-1(23)-3 | m/z = 550.10(C₃₅H₂₃BrN₂ = 551.47) | Sub1-1(24)-3 | m/z = 551.10(C₃₄H₂₂BrN₃ = 552.46) |
| Sub1-1(25)-3 | m/z = 551.10(C₃₄H₂₂BrN₃ = 552.46 | Sub1-1(26)-3 | m/z = 552.09(C₃₃H₂₁BrN₄ = 553.45) |
| Sub1-1(27)-3 | m/z = 552.09(C₃₃H₂₁BrN₄ = 553.45) | Sub1-1(28)-3 | m/z = 449.05(C₂₆H₁₆BrN₃ = 450.33) |

### Synthesis of Sub 1-3

A two-necked RBF was equipped with a dropping-funnel, and the product was dissolved in 500 ml of THF and the temperature was maintained at −78□. After stirring for 1 h, trimethoxyborate was slowly added dropwise, followed by again stirring for 1 h. Upon the completion of the reaction, 500 ml of 5% hydrochloric acid was added, followed by stirring at room temperature for 1 h, extraction with water and ethyl acetate, concentration, and recrystallization with MC and Hexane, thereby obtaining compound Sub 1-3.

### Synthesis of Sub 1(1)-3

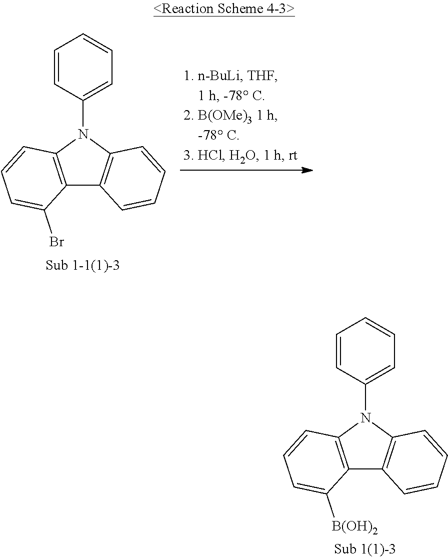 [SMILES: Brc1cccc2c1c1ccccc1n2-c1ccccc1 ; OB(O)c1cccc2c1c1ccccc1n2-c1ccccc1]

A two-necked RBF was equipped with a dropping-funnel, and Sub 1-1(1) (38 g, 118 mmol) was dissolved in 500 ml of THF and the temperature was maintained at −78□. After stirring for 1 h, trimethoxyborate (18.4 g, 177 mmol) was slowly added dropwise, followed by again stirring for 1 h. Upon the completion of the reaction, 500 ml of 5% hydrochloric acid was added, followed by stirring at room temperature for 1 h, extraction with water and ethyl acetate, concentration, and recrystallization with MC and Hexane, thereby obtaining 20.3 g of compound Sub 1(1)-3 (yield: 60%).

Examples of Sub 1-3 are as follows, but are limited thereto, and FD-MS values thereof are shown in table 3-2 below.

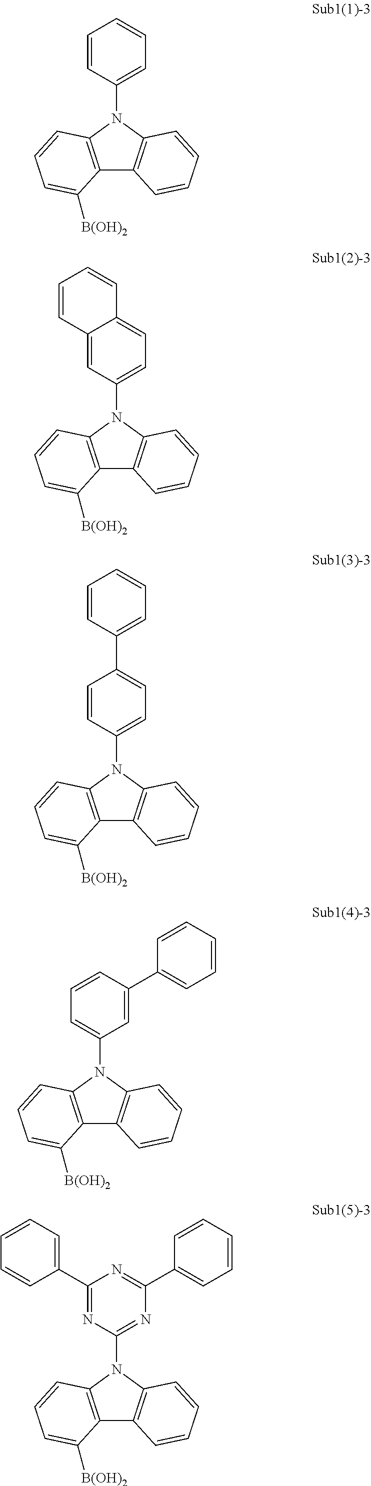

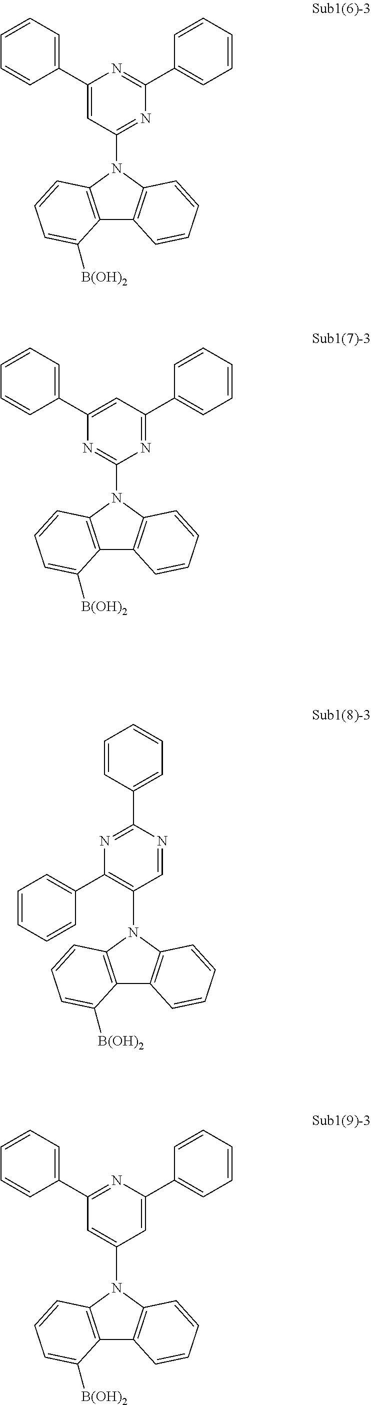 [SMILES: OB(O)c1cccc2c1c1ccccc1n2-c1cc(-c2ccccc2)nc(-c2ccccc2)n1 ; OB(O)c1cccc2c1c1ccccc1n2-c1nc(-c2ccccc2)cc(-c2ccccc2)n1 ; OB(O)c1cccc2c1c1ccccc1n2-c1cnc(-c2ccccc2)nc1-c1ccccc1 ; OB(O)c1cccc2c1c1ccccc1n2-c1cc(-c2ccccc2)nc(-c2ccccc2)c1]

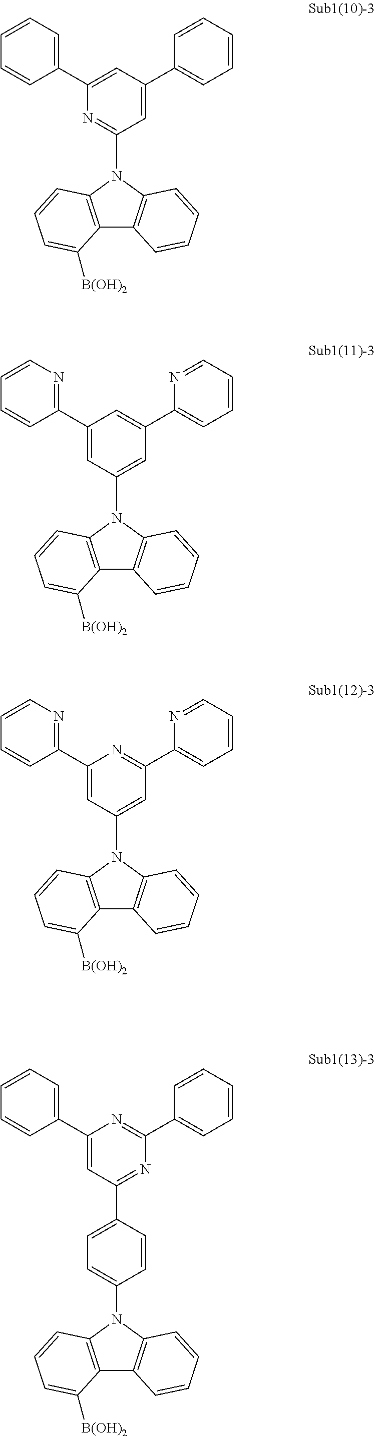 [SMILES: OB(O)c1cccc2c1c1ccccc1n2-c1cc(-c2ccccc2)cc(-c2ccccc2)n1 ; OB(O)c1cccc2c1c1ccccc1n2-c1cc(-c2ccccn2)cc(-c2ccccn2)c1 ; OB(O)c1cccc2c1c1ccccc1n2-c1cc(-c2ccccn2)nc(-c2ccccn2)c1 ; OB(O)c1cccc2c1c1ccccc1n2-c1ccc(-c2cc(-c3ccccc3)nc(-c3ccccc3)n2)cc1]

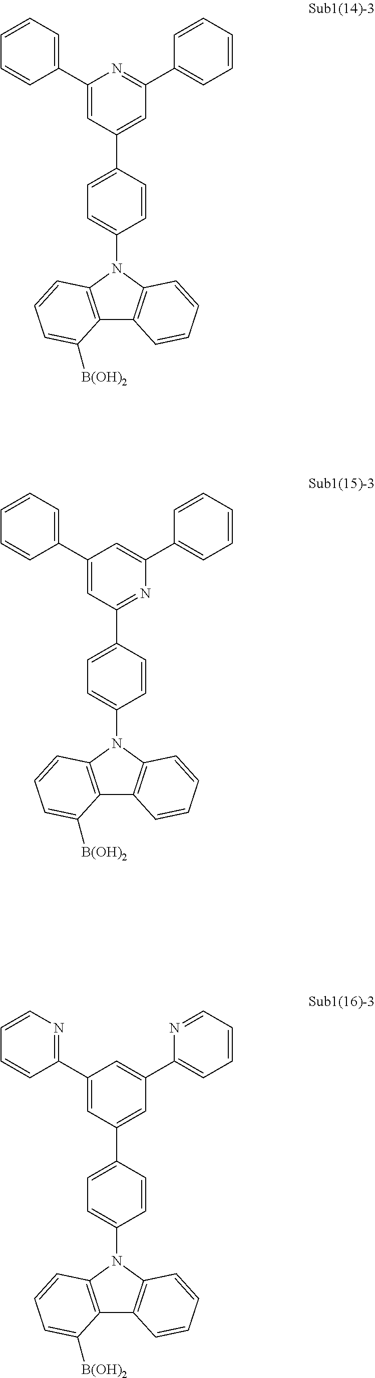 [SMILES: OB(O)c1cccc2c1c1ccccc1n2-c1ccc(-c2cc(-c3ccccc3)nc(-c3ccccc3)c2)cc1 ; OB(O)c1cccc2c1c1ccccc1n2-c1ccc(-c2cc(-c3ccccc3)cc(-c3ccccc3)n2)cc1 ; OB(O)c1cccc2c1c1ccccc1n2-c1ccc(-c2cc(-c3ccccn3)cc(-c3ccccn3)c2)cc1]

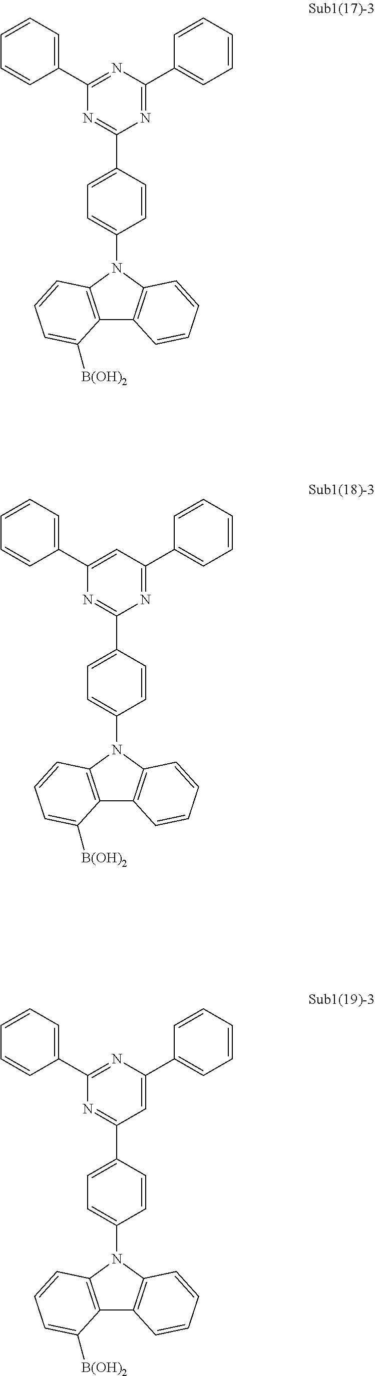 [SMILES: OB(O)c1cccc2c1c1ccccc1n2-c1ccc(-c2nc(-c3ccccc3)nc(-c3ccccc3)n2)cc1 ; OB(O)c1cccc2c1c1ccccc1n2-c1ccc(-c2nc(-c3ccccc3)cc(-c3ccccc3)n2)cc1 ; OB(O)c1cccc2c1c1ccccc1n2-c1ccc(-c2cc(-c3ccccc3)nc(-c3ccccc3)n2)cc1]

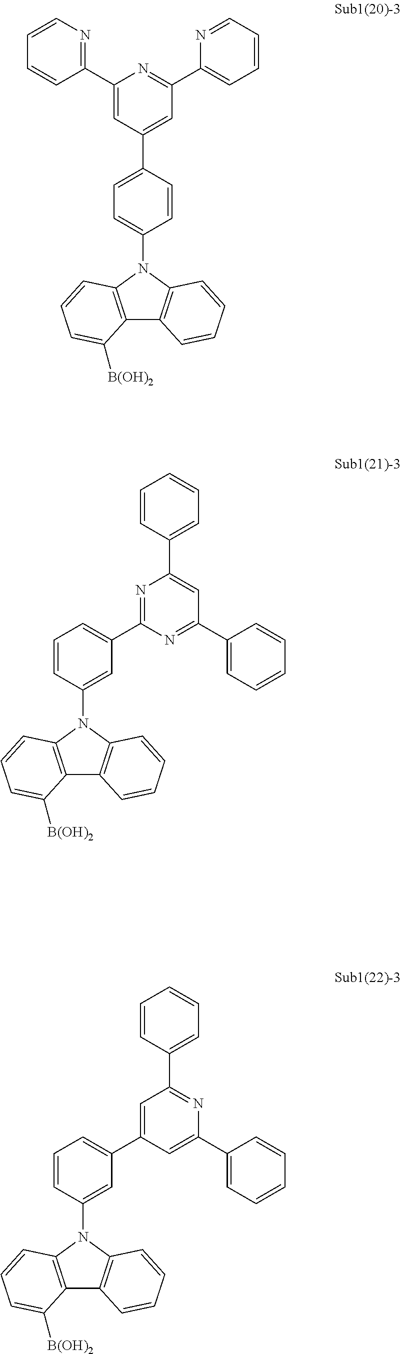 [SMILES: OB(O)c1cccc2c1c1ccccc1n2-c1ccc(-c2cc(-c3ccccn3)nc(-c3ccccn3)c2)cc1 ; OB(O)c1cccc2c1c1ccccc1n2-c1cccc(-c2nc(-c3ccccc3)cc(-c3ccccc3)n2)c1 ; OB(O)c1cccc2c1c1ccccc1n2-c1cccc(-c2cc(-c3ccccc3)nc(-c3ccccc3)c2)c1]

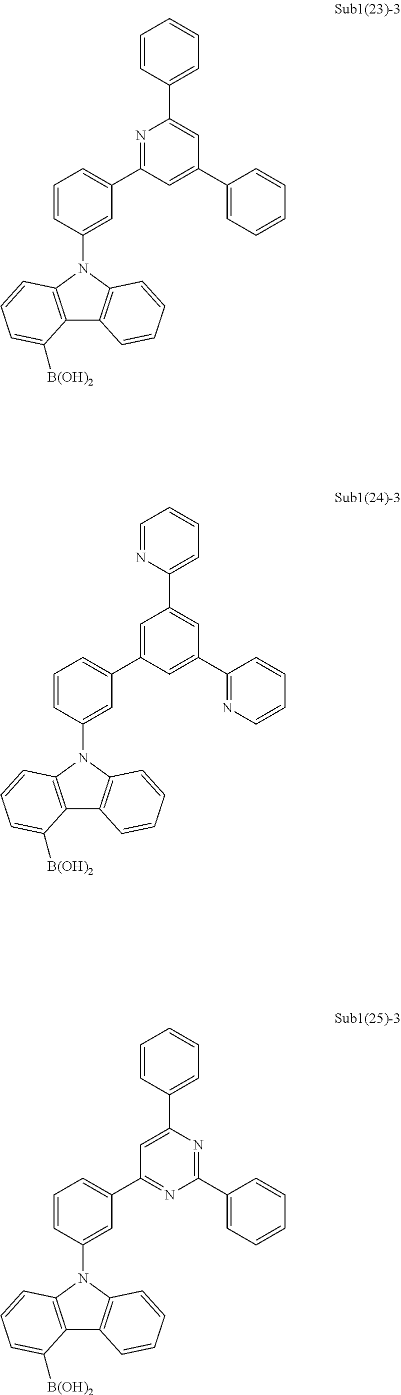 [SMILES: OB(O)c1cccc2c1c1ccccc1n2-c1cccc(-c2cc(-c3ccccc3)cc(-c3ccccc3)n2)c1 ; OB(O)c1cccc2c1c1ccccc1n2-c1cccc(-c2cc(-c3ccccn3)cc(-c3ccccn3)c2)c1 ; OB(O)c1cccc2c1c1ccccc1n2-c1cccc(-c2cc(-c3ccccc3)nc(-c3ccccc3)n2)c1]

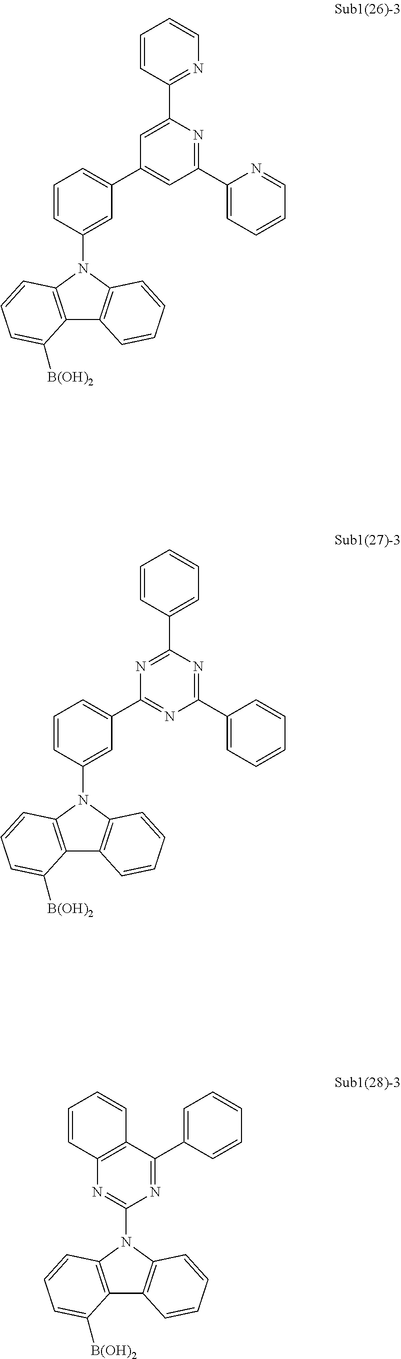 [SMILES: OB(O)c1cccc2c1c1ccccc1n2-c1cccc(-c2cc(-c3ccccn3)nc(-c3ccccn3)c2)c1 ; OB(O)c1cccc2c1c1ccccc1n2-c1cccc(-c2nc(-c3ccccc3)nc(-c3ccccc3)n2)c1 ; OB(O)c1cccc2c1c1ccccc1n2-c1nc(-c2ccccc2)c2ccccc2n1]

TABLE 3-2

| Compound | FD-MS | Compound | FD-MS |
|---|---|---|---|
| Sub 1(1)-3 | m/z = 287.11(C₁₈H₁₄BNO₂ = 287.12) | Sub 1(2)-3 | m/z = 337.13(C₂₂H₁₈BNO₂ = 337.18) |
| Sub 1(3)-3 | m/z = 363.14(C₂₄H₁₈BNO₂ = 363.22) | Sub 1(4)-3 | m/z = 363.14(C₂₄H₁₈BNO₂ = 363.22) |
| Sub 1(5)-3 | m/z = 442.16(C₂₇H₁₉BN₄O₂ = 442.28) | Sub 1(6)-3 | m/z = 441.16(C₂₅H₂₀BN₃O₂ = 441.29) |
| Sub 1(7)-3 | m/z = 441.16(C₂₃H₂₀BN₃O₂ = 441.29) | Sub 1(8)-3 | m/z = 441.16(C₂₈H₂₀BN₃O₂ = 441.29) |
| Sub 1(9)-3 | m/z = 440.17(C₃₉H₂₁BN₂O₂ = 440.30) | Sub 1(10)-3 | m/z = 440.17(C₂₉H₂₁BN₂O₂ = 440.30) |
| Sub 1(11)-3 | m/z = 441.16(C₂₈H₂₀BN₃O₂ = 441.29) | Sub 1(12)-3 | m/z = 442.16(C₂₇H₁₉BN₄O₂ = 442.28) |
| Sub 1(13)-3 | m/z = 517.20(C₃₄H₂₄BN₃O₂ = 517.38) | Sub 1(14)-3 | m/z = 516.20(C₃₅H₂₅BN₂O₂ = 516.40) |
| Sub 1(15)-3 | m/z = 516.20(C₃₅H₂₅BN₂O₂ = 516.40) | Sub 1(16)-3 | m/z = 517.20(C₃₄H₂₄BN₃O₂ = 517.38) |
| Sub 1(17)-3 | m/z = 518.19(C₃₅H₂₃BN₄O₂ = 518.37) | Sub 1(18)-3 | m/z = 517.20(C₃₄H₂₄BN₃O₂ = 517.38) |
| Sub 1(19)-3 | m/z = 517.20(C₃₄H₂₄BN₃O₂ = 517.38) | Sub 1(20)-3 | m/z = 518.19(C₃₃H₂₃BN₄O₂ = 518.37) |
| Sub 1(21)-3 | m/z = 517.20(C₃₄H₂₄BN₃O₂ = 517.38) | Sub 1(22)-3 | m/z = 516.20(C₃₅H₂₅BN₂O₂ = 516.40) |
| Sub 1(23)-3 | m/z = 516.20(C₃₅H₂₅BN₂O₂ = 516.40) | Sub 1(24)-3 | m/z = 517.20(C₃₄H₂₄BN₃O₂ = 517.38) |
| Sub 1(25)-3 | m/z = 517.20(C₃₄H₂₄BN₃O₂ = 517.38) | Sub 1(26)-3 | m/z = 518.19(C₃₃H₂₃BN₄O₂ = 518.37) |
| Sub 1(27)-3 | m/z = 518.19(C₃₃H₂₃BN₄O₂ = 518.37) | Sub 1(28)-3 | m/z = 415.15(C₂₆H₁₈BN₃O₂ = 415.25) |

### □. Synthesis Example of Sub 2

Sub 3-1 in Reaction Scheme 3-1 may be synthesized via the reaction pathway of Reaction Scheme 3-5 below, but is not limited thereto.

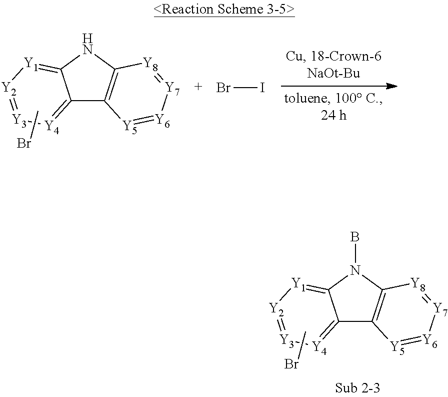

### Synthesis of Sub 2-1(1)-3

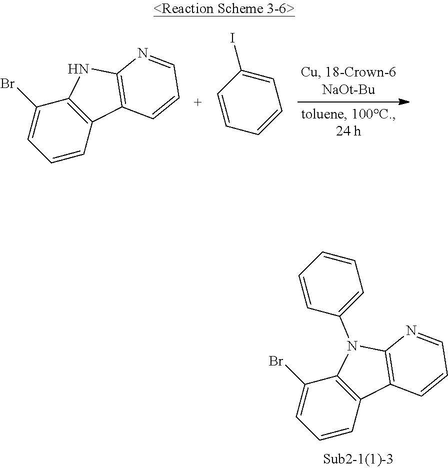

After 8-bromo-9H-pyrido[2,3-b]indole (50.2 g, 203 mmol) and iodobenzene (49.0 g, 240 mmol) were mixed with 800 mL of toluene, Cu (764 mg, 12 mmol), 18-Crown-6 (6.3 g, 24 mmol), and NaOt-Bu (57.6 g, 600 mmol) were added thereto, and the mixture was stirred under reflux at 100□ for 24 h.

After extraction with ether and water, the organic layer was dried over MgSO₄ and concentrated, and then the generated organic material was subjected to silica gel column chromatography and recrystallization to give 28.2 g of 8-bromo-9-phenyl-9H-pyrido[2,3-b]indole (yield: 43%).

Examples of Sub 2-3 are as follows, but are limited thereto, and FD-MS values thereof are shown in table 3-3 below.

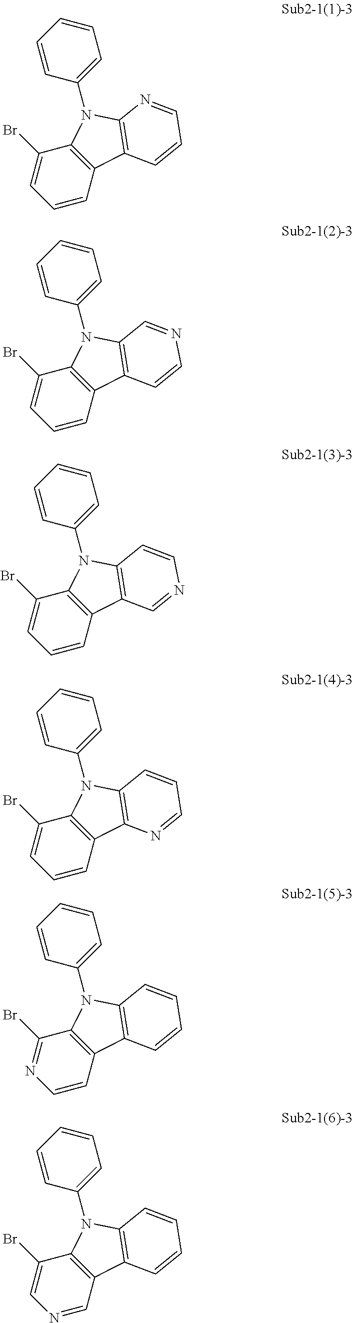

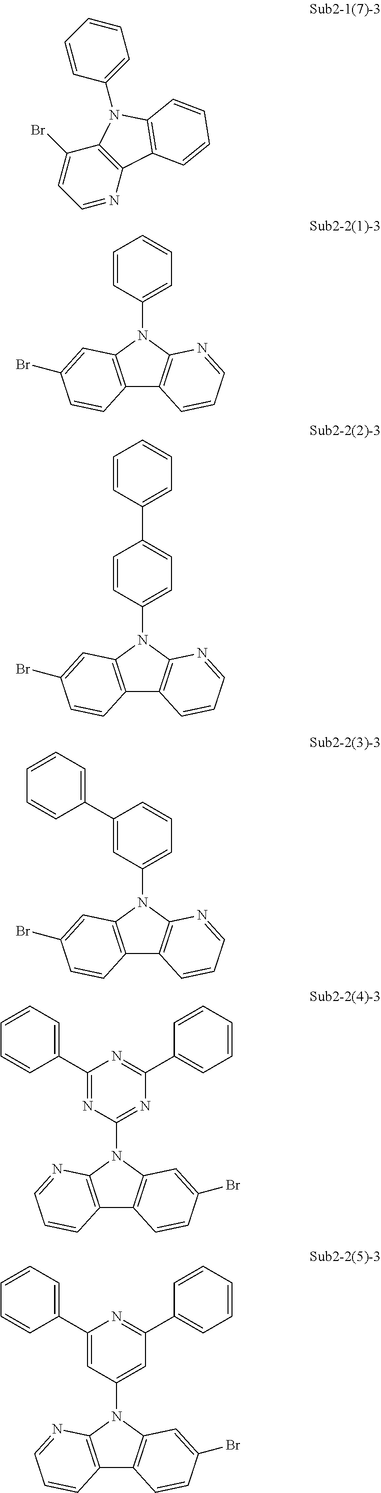

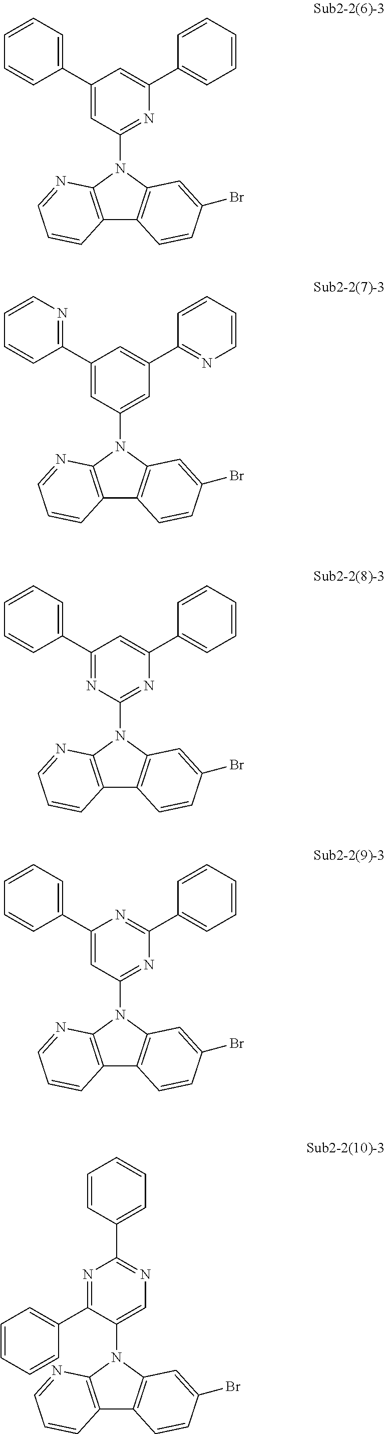 [SMILES: Brc1ccc2c3cccnc3n(-c3cc(-c4ccccc4)cc(-c4ccccc4)n3)c2c1 ; Brc1ccc2c3cccnc3n(-c3cc(-c4ccccn4)cc(-c4ccccn4)c3)c2c1 ; Brc1ccc2c3cccnc3n(-c3nc(-c4ccccc4)cc(-c4ccccc4)n3)c2c1 ; Brc1ccc2c3cccnc3n(-c3cc(-c4ccccc4)nc(-c4ccccc4)n3)c2c1 ; Brc1ccc2c3cccnc3n(-c3cnc(-c4ccccc4)nc3-c3ccccc3)c2c1]

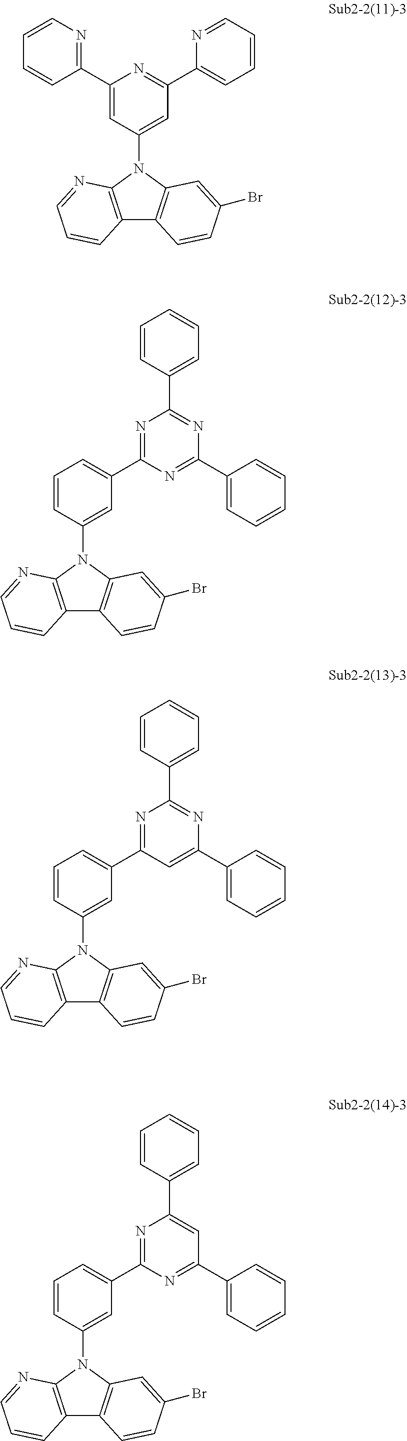 [SMILES: Brc1ccc2c3cccnc3n(-c3cc(-c4ccccn4)nc(-c4ccccn4)c3)c2c1 ; Brc1ccc2c3cccnc3n(-c3cccc(-c4nc(-c5ccccc5)nc(-c5ccccc5)n4)c3)c2c1 ; Brc1ccc2c3cccnc3n(-c3cccc(-c4cc(-c5ccccc5)nc(-c5ccccc5)n4)c3)c2c1 ; Brc1ccc2c3cccnc3n(-c3cccc(-c4nc(-c5ccccc5)cc(-c5ccccc5)n4)c3)c2c1]

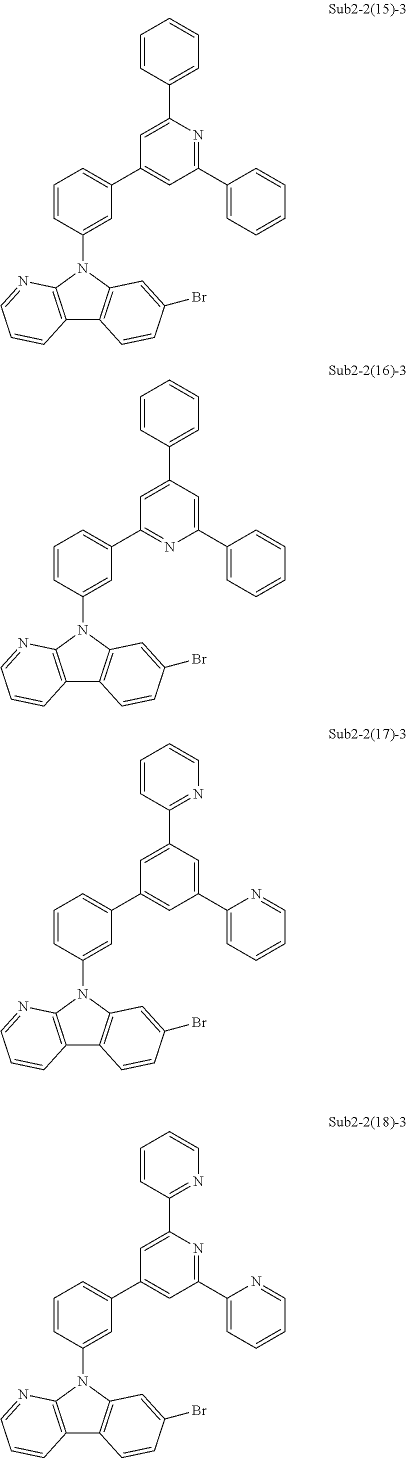

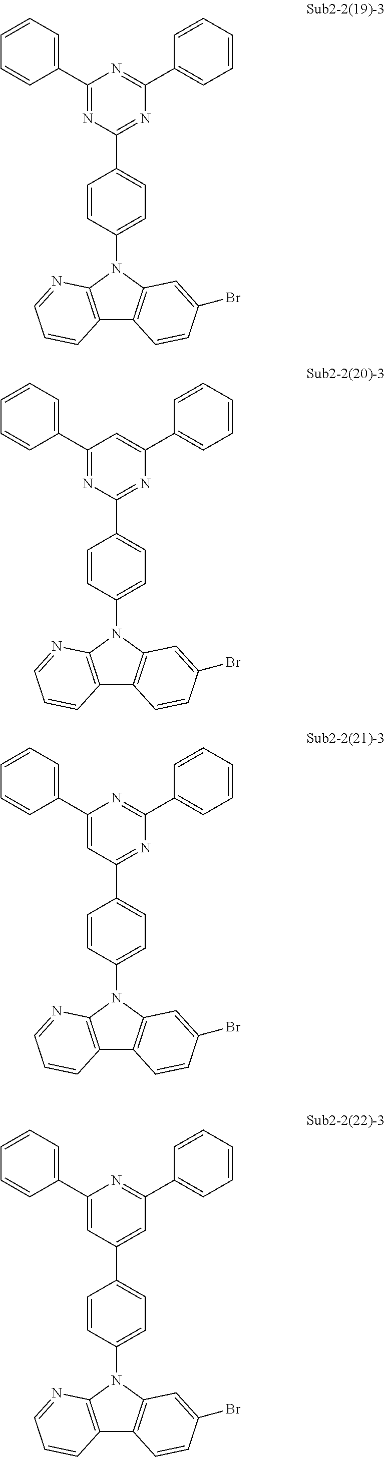 [SMILES: Brc1ccc2c3cccnc3n(-c3ccc(-c4nc(-c5ccccc5)nc(-c5ccccc5)n4)cc3)c2c1 ; Brc1ccc2c3cccnc3n(-c3ccc(-c4nc(-c5ccccc5)cc(-c5ccccc5)n4)cc3)c2c1 ; Brc1ccc2c3cccnc3n(-c3ccc(-c4cc(-c5ccccc5)nc(-c5ccccc5)n4)cc3)c2c1 ; Brc1ccc2c3cccnc3n(-c3ccc(-c4cc(-c5ccccc5)nc(-c5ccccc5)c4)cc3)c2c1]

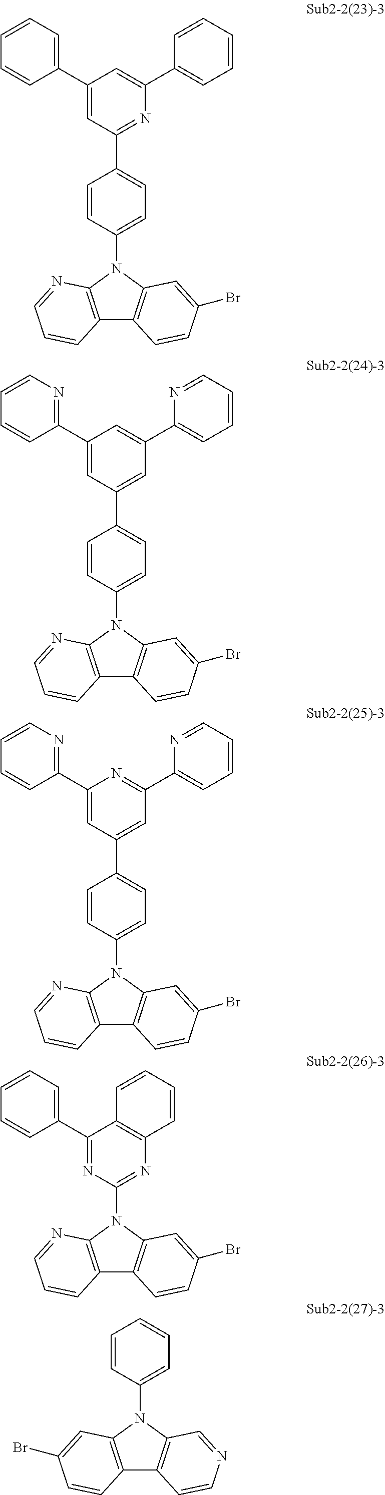

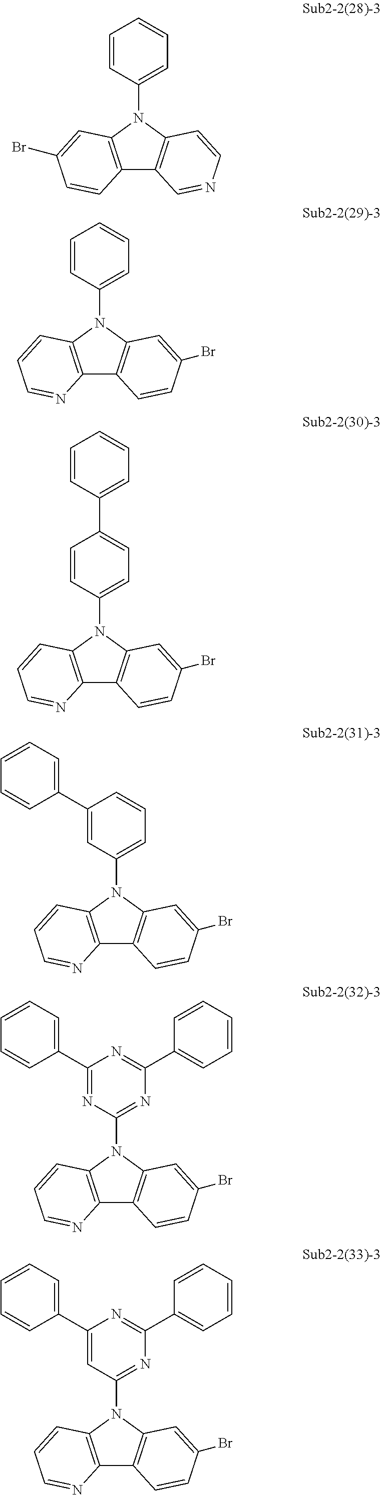

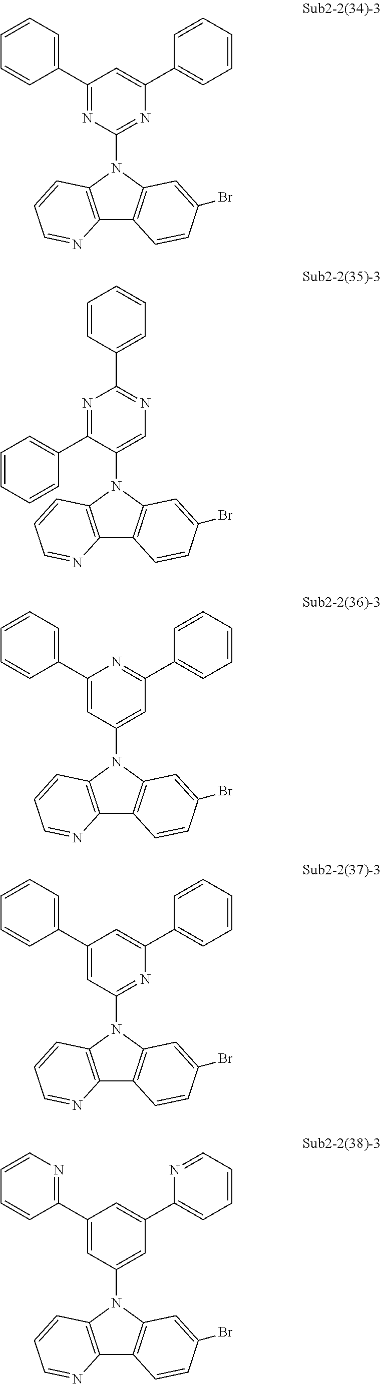 [SMILES: Brc1ccc2c3ncccc3n(-c3nc(-c4ccccc4)cc(-c4ccccc4)n3)c2c1 ; Brc1ccc2c3ncccc3n(-c3cnc(-c4ccccc4)nc3-c3ccccc3)c2c1 ; Brc1ccc2c3ncccc3n(-c3cc(-c4ccccc4)nc(-c4ccccc4)c3)c2c1 ; Brc1ccc2c3ncccc3n(-c3cc(-c4ccccc4)cc(-c4ccccc4)n3)c2c1 ; Brc1ccc2c3ncccc3n(-c3cc(-c4ccccn4)cc(-c4ccccn4)c3)c2c1]

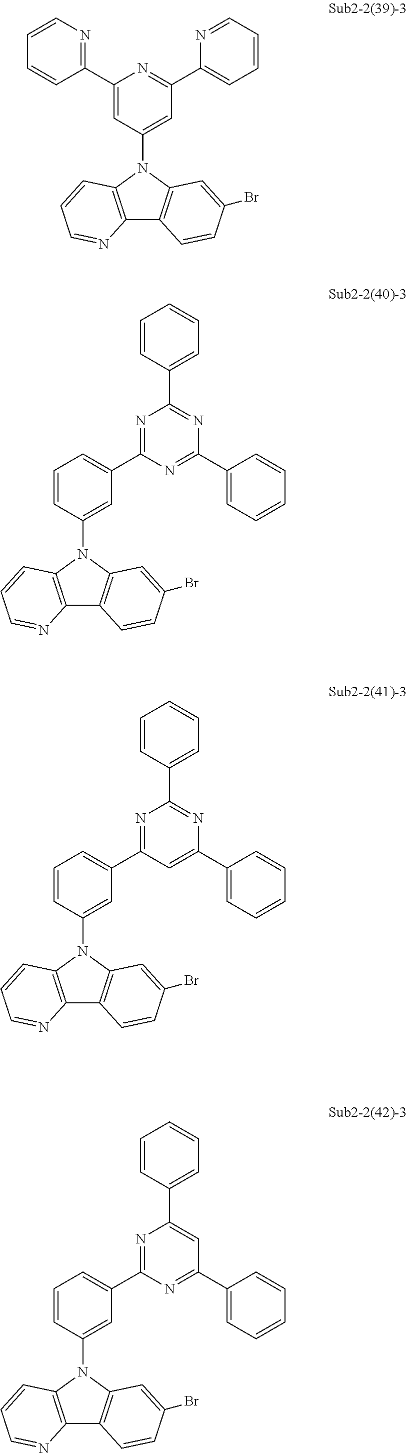 [SMILES: Brc1ccc2c3ncccc3n(-c3cc(-c4ccccn4)nc(-c4ccccn4)c3)c2c1 ; Brc1ccc2c3ncccc3n(-c3cccc(-c4nc(-c5ccccc5)nc(-c5ccccc5)n4)c3)c2c1 ; Brc1ccc2c3ncccc3n(-c3cccc(-c4cc(-c5ccccc5)nc(-c5ccccc5)n4)c3)c2c1 ; Brc1ccc2c3ncccc3n(-c3cccc(-c4nc(-c5ccccc5)cc(-c5ccccc5)n4)c3)c2c1]

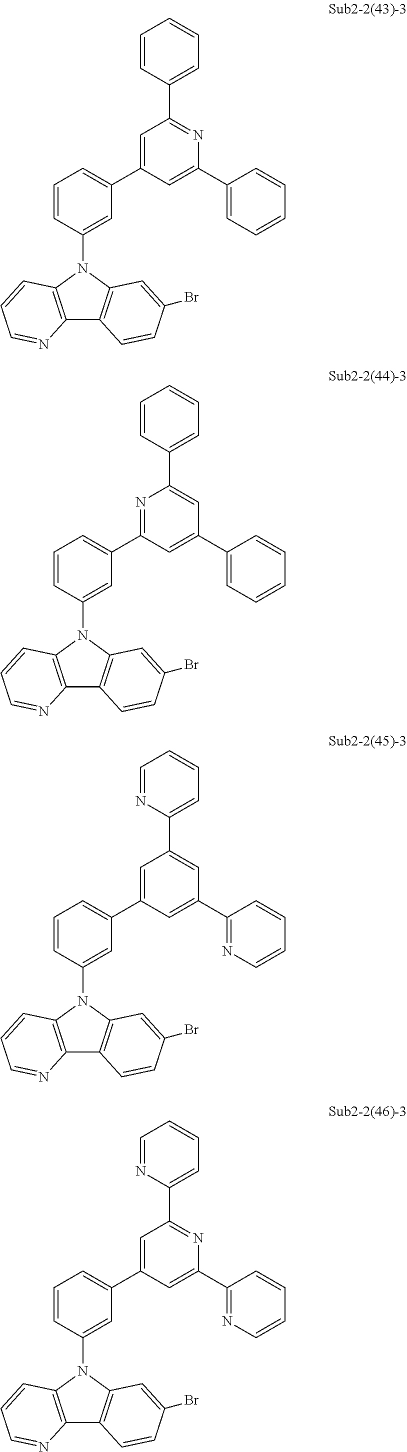 [SMILES: Brc1ccc2c3ncccc3n(-c3cccc(-c4cc(-c5ccccc5)nc(-c5ccccc5)c4)c3)c2c1 ; Brc1ccc2c3ncccc3n(-c3cccc(-c4cc(-c5ccccc5)cc(-c5ccccc5)n4)c3)c2c1 ; Brc1ccc2c3ncccc3n(-c3cccc(-c4cc(-c5ccccn5)cc(-c5ccccn5)c4)c3)c2c1 ; Brc1ccc2c3ncccc3n(-c3cccc(-c4cc(-c5ccccn5)nc(-c5ccccn5)c4)c3)c2c1]

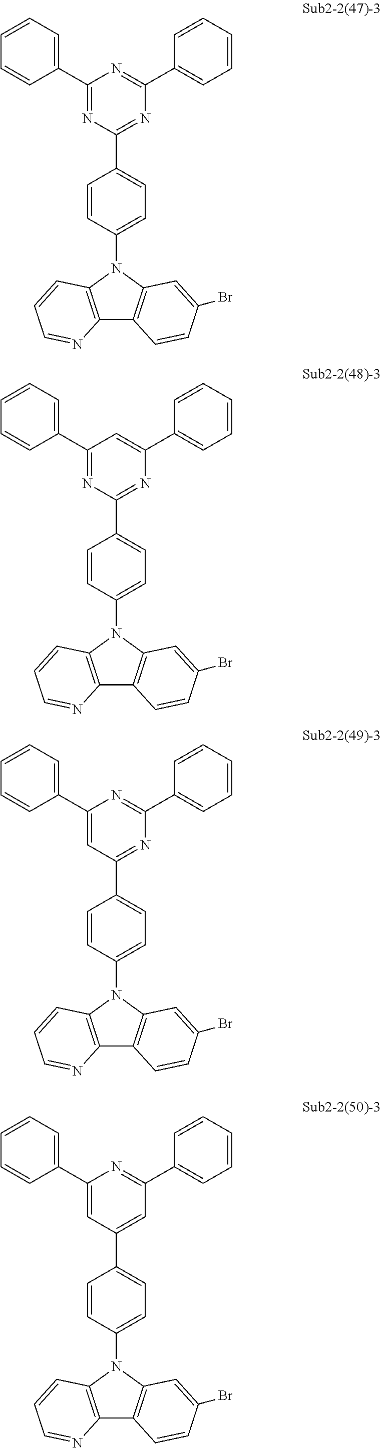

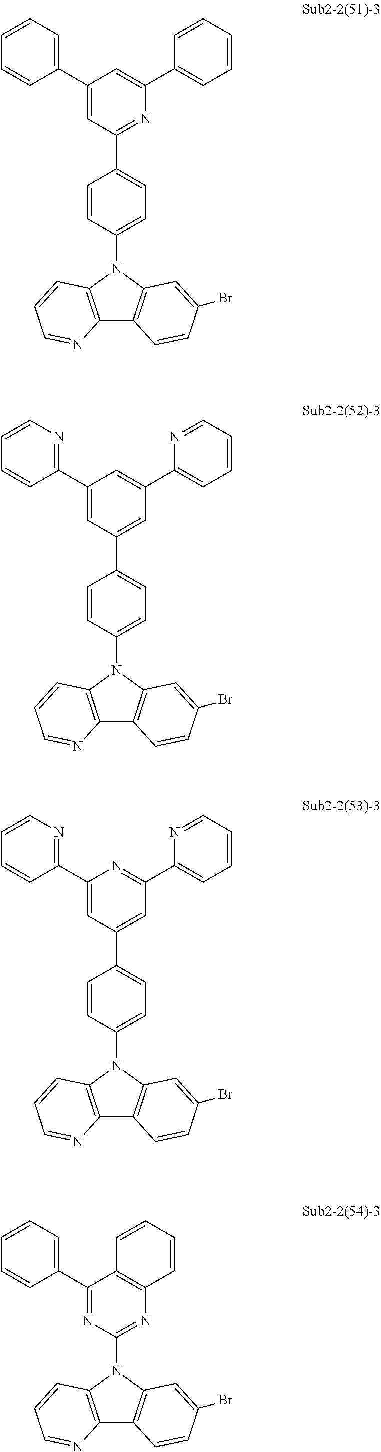 [SMILES: Brc1ccc2c3ncccc3n(-c3ccc(-c4cc(-c5ccccc5)cc(-c5ccccc5)n4)cc3)c2c1 ; Brc1ccc2c3ncccc3n(-c3ccc(-c4cc(-c5ccccn5)cc(-c5ccccn5)c4)cc3)c2c1 ; Brc1ccc2c3ncccc3n(-c3ccc(-c4cc(-c5ccccn5)nc(-c5ccccn5)c4)cc3)c2c1 ; Brc1ccc2c3ncccc3n(-c3nc(-c4ccccc4)c4ccccc4n3)c2c1]

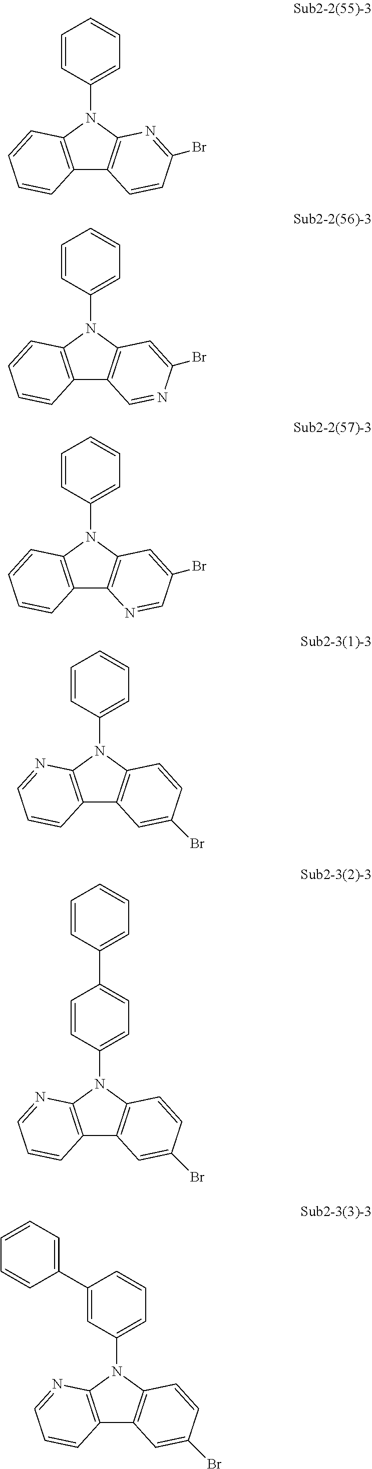 [SMILES: Brc1ccc2c3ccccc3n(-c3ccccc3)c2n1 ; Brc1cc2c(cn1)c1ccccc1n2-c1ccccc1 ; Brc1cnc2c3ccccc3n(-c3ccccc3)c2c1 ; Brc1ccc2c(c1)c1cccnc1n2-c1ccccc1 ; Brc1ccc2c(c1)c1cccnc1n2-c1ccc(-c2ccccc2)cc1 ; Brc1ccc2c(c1)c1cccnc1n2-c1cccc(-c2ccccc2)c1]

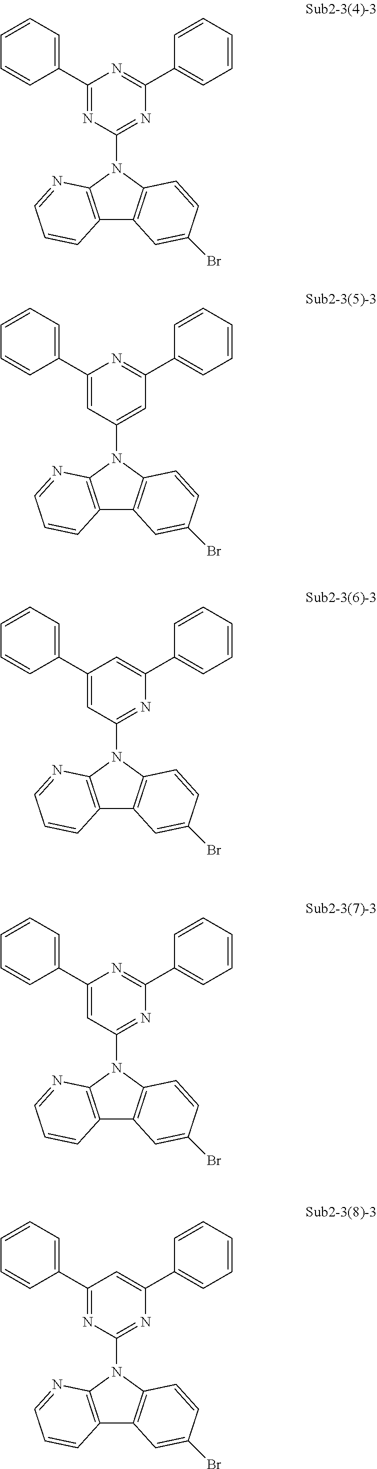 [SMILES: Brc1ccc2c(c1)c1cccnc1n2-c1nc(-c2ccccc2)nc(-c2ccccc2)n1 ; Brc1ccc2c(c1)c1cccnc1n2-c1cc(-c2ccccc2)nc(-c2ccccc2)c1 ; Brc1ccc2c(c1)c1cccnc1n2-c1cc(-c2ccccc2)cc(-c2ccccc2)n1 ; Brc1ccc2c(c1)c1cccnc1n2-c1cc(-c2ccccc2)nc(-c2ccccc2)n1 ; Brc1ccc2c(c1)c1cccnc1n2-c1nc(-c2ccccc2)cc(-c2ccccc2)n1]

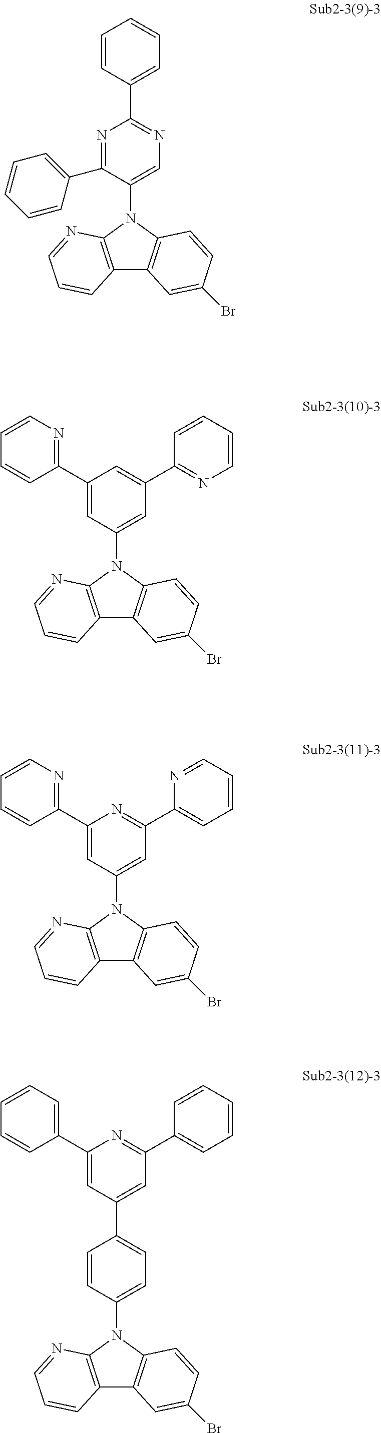 [SMILES: Brc1ccc2c(c1)c1cccnc1n2-c1cnc(-c2ccccc2)nc1-c1ccccc1 ; Brc1ccc2c(c1)c1cccnc1n2-c1cc(-c2ccccn2)cc(-c2ccccn2)c1 ; Brc1ccc2c(c1)c1cccnc1n2-c1cc(-c2ccccn2)nc(-c2ccccn2)c1 ; Brc1ccc2c(c1)c1cccnc1n2-c1ccc(-c2cc(-c3ccccc3)nc(-c3ccccc3)c2)cc1]

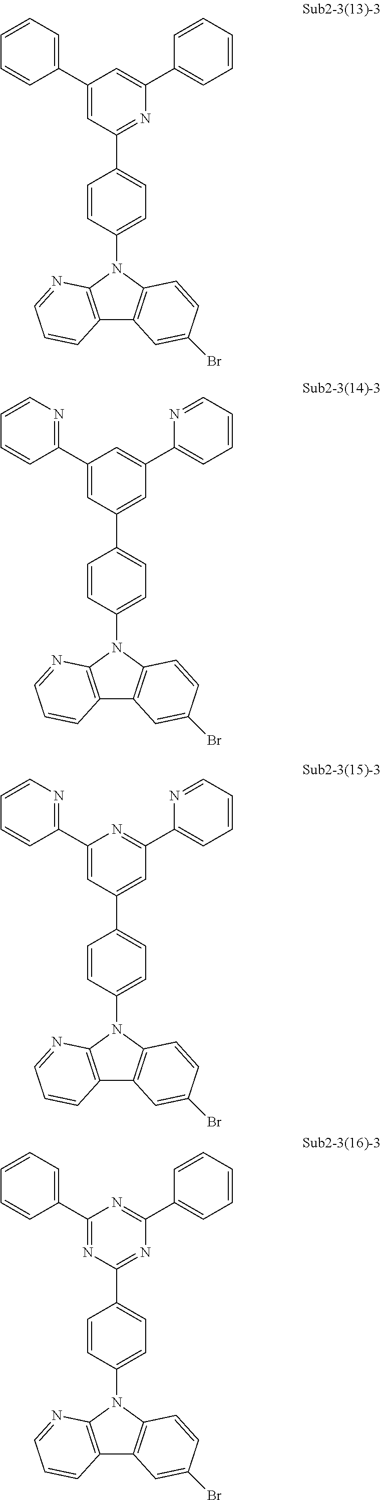

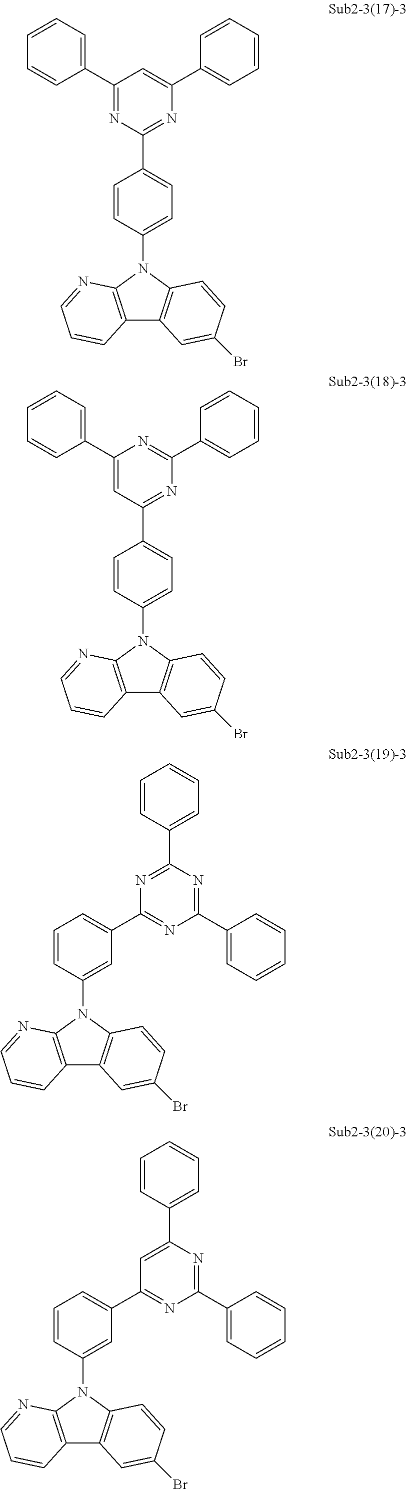

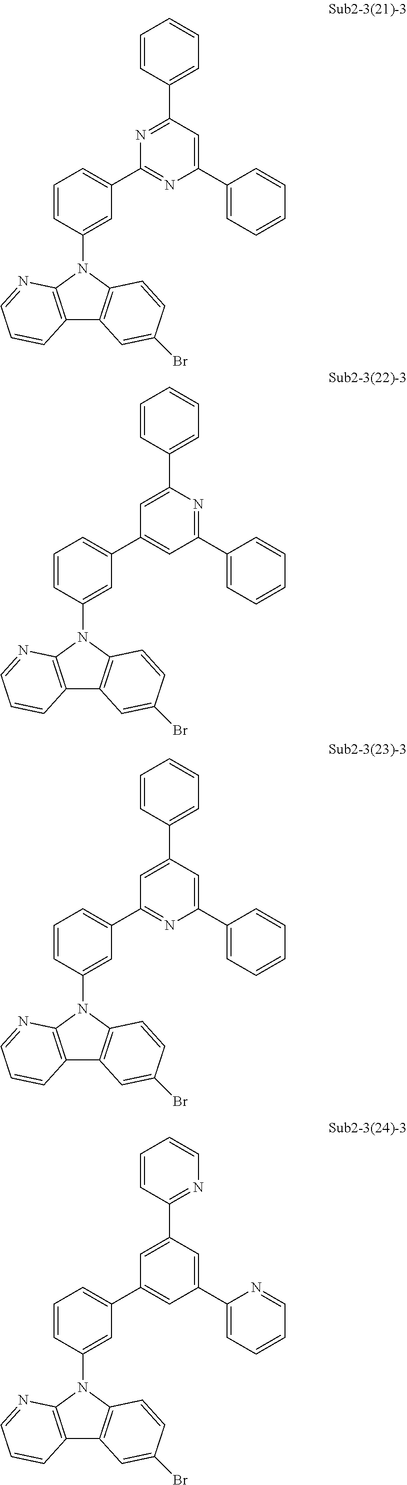

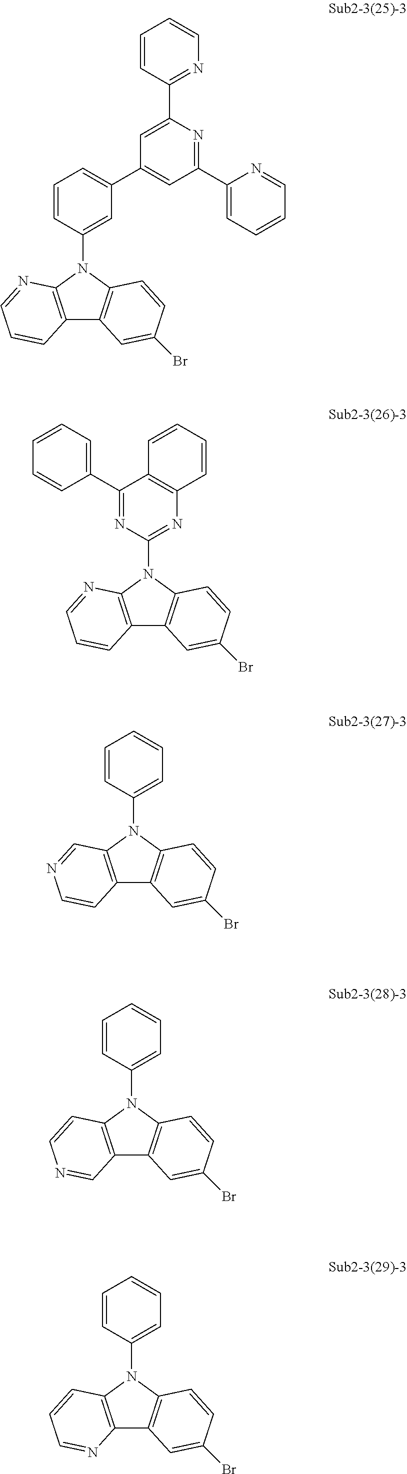 [SMILES: Brc1ccc2c(c1)c1cccnc1n2-c1cccc(-c2cc(-c3ccccn3)nc(-c3ccccn3)c2)c1 ; Brc1ccc2c(c1)c1cccnc1n2-c1nc(-c2ccccc2)c2ccccc2n1 ; Brc1ccc2c(c1)c1ccncc1n2-c1ccccc1 ; Brc1ccc2c(c1)c1cnccc1n2-c1ccccc1 ; Brc1ccc2c(c1)c1ncccc1n2-c1ccccc1]

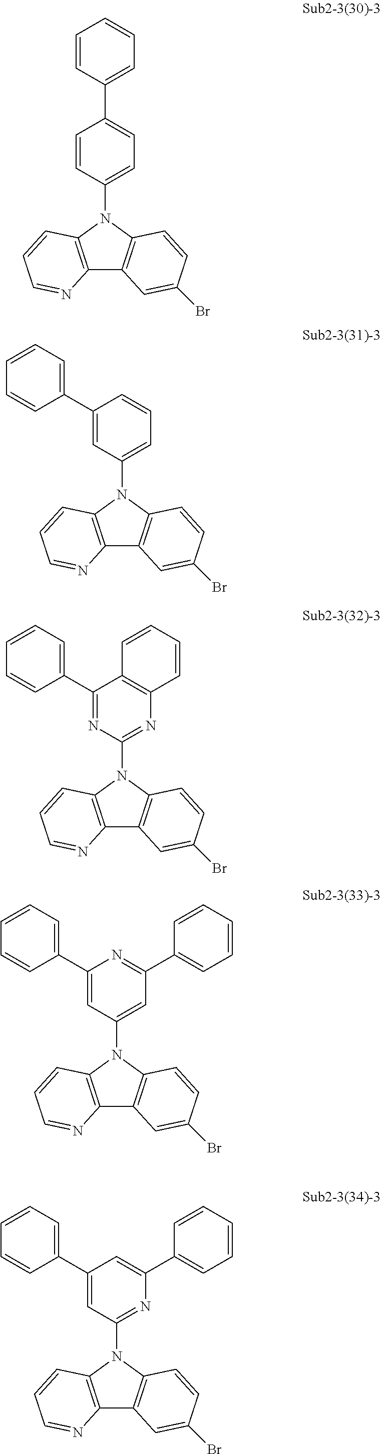

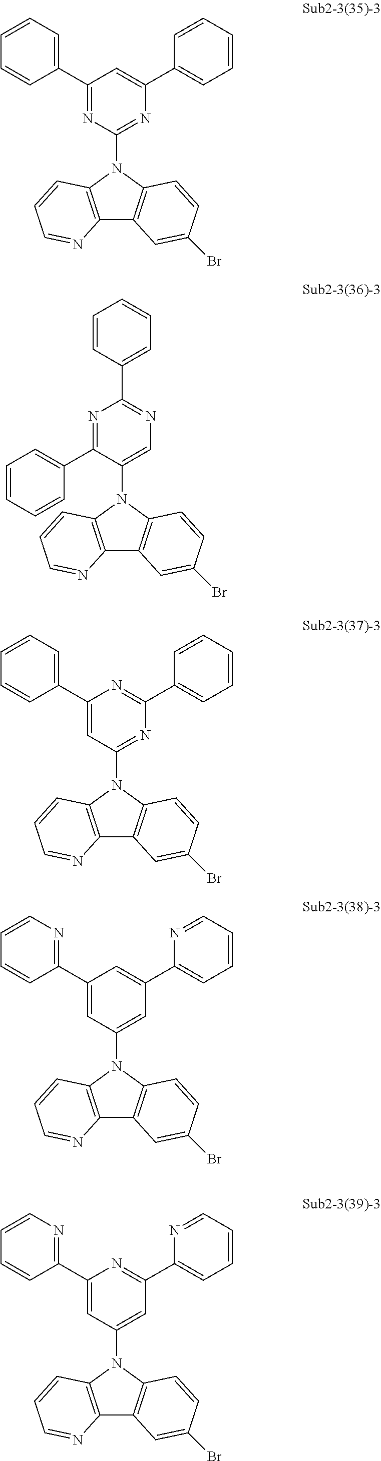

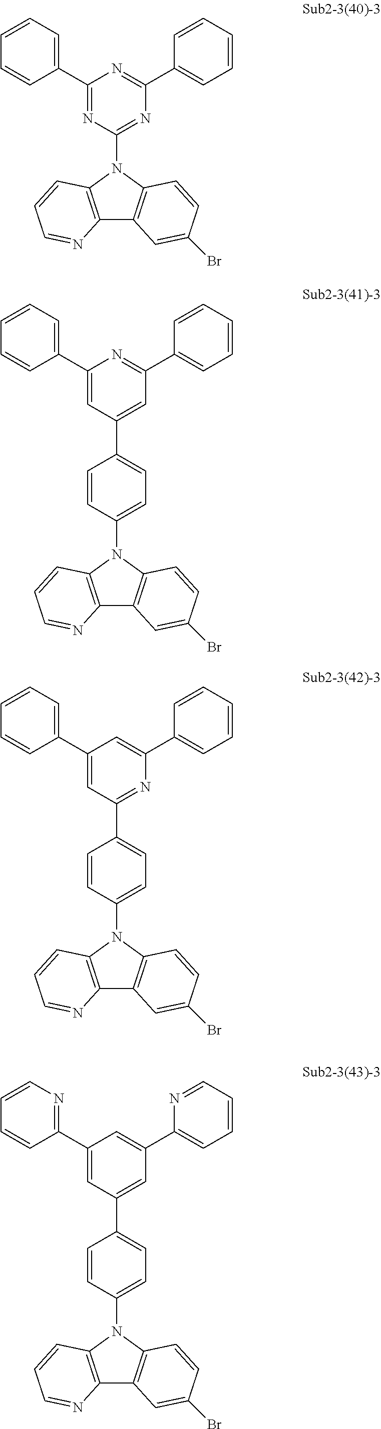

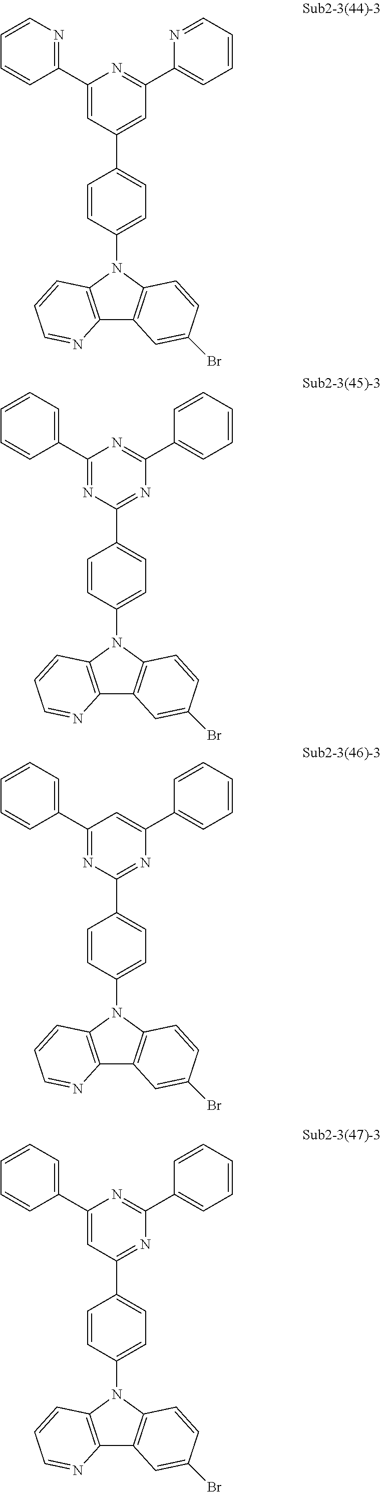

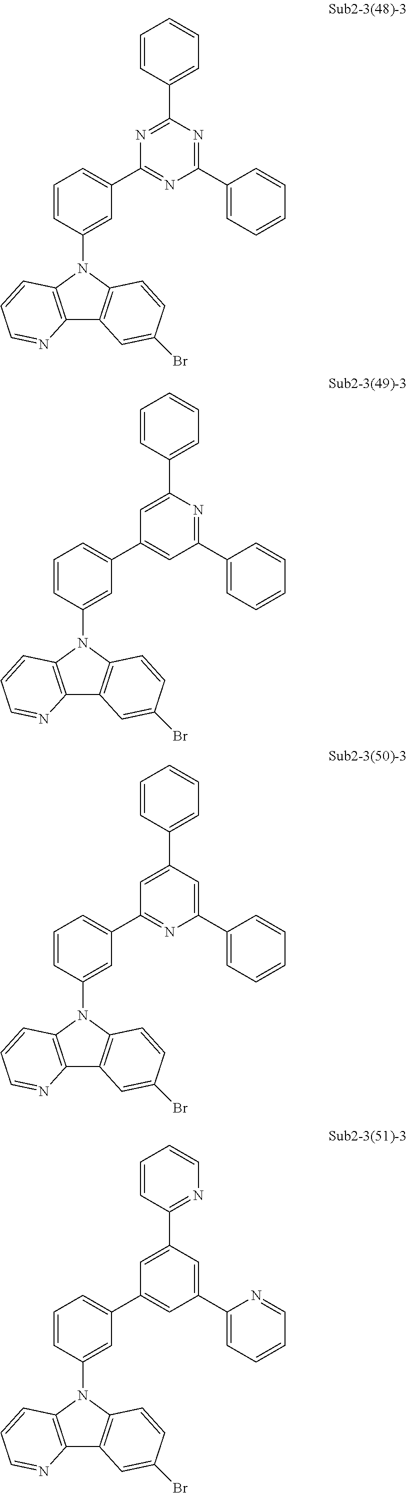

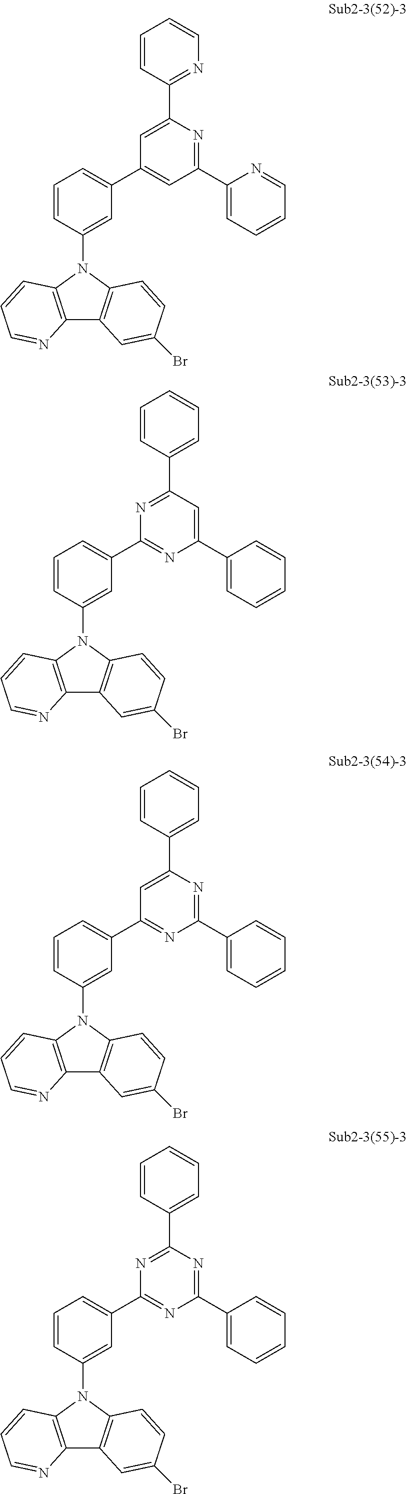

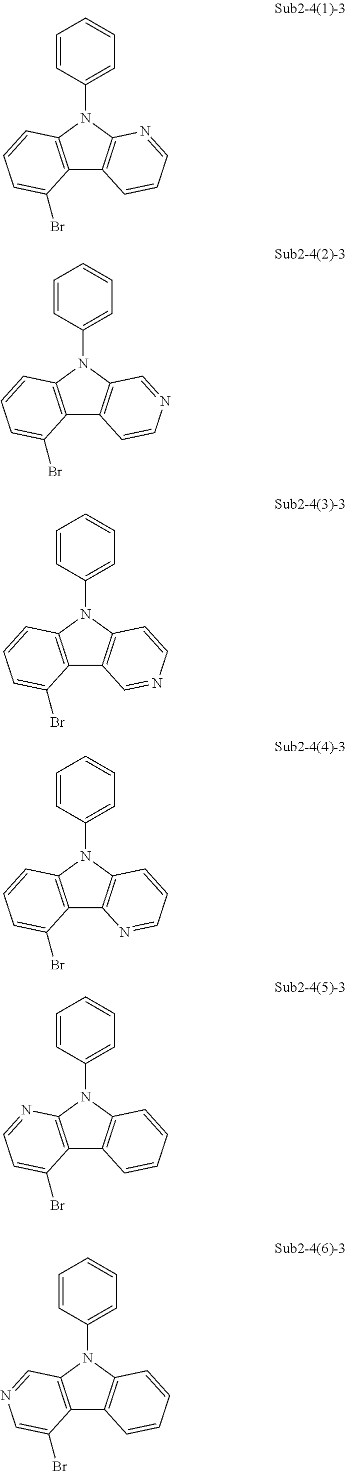

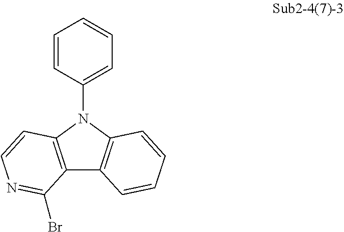 [SMILES: Brc1nccc2c1c1ccccc1n2-c1ccccc1]

TABLE 3-3

| Compound | FD-MS | Compound | FD-MS |
|---|---|---|---|
| Sub2-1(1)-3 | m/z = 322.01(C₁₇H₁₁BrN₂ = 323.19) | Sub2-1(2)-3 | m/z = 322.01(C₁₇H₁₁BrN₂ = 323.19) |
| Sub2-1(3)-3 | m/z = 322.01(C₁₇H₁₁BrN₂ = 323.19) | Sub2-1(4)-3 | m/z = 322.01(C₁₇H₁₁BrN₂ = 323.19) |
| Sub2-1(5)-3 | m/z = 322.01(C₁₇H₁₁BrN₂ = 323.19) | Sub2-1(6)-3 | m/z = 322.01(C₁₇H₁₁BrN₂ = 323.19) |
| Sub2-1(7)-3 | m/z = 322.01(C₁₇H₁₁BrN₂ = 323.19) | Sub2-2(1)-3 | m/z = 322.01(C₁₇H₁₁BrN₂ = 323.19) |
| Sub2-2(2)-3 | m/z = 398.04(C₂₃H₁₅BrN₂ = 399.28) | Sub2-2(3)-3 | m/z = 398.04(C₂₃H₁₅BrN₂ = 399.28) |
| Sub2-2(4)-3 | m/z = 477.06(C₂₆H₁₆BrN₅ = 478.34) | Sub2-2(5)-3 | m/z = 475.07(C₂₈H₁₈BrN₃ = 476.37) |
| Sub2-2(6)-3 | m/z = 475.07(C₂₈H₁₈BrN₃ = 476.37) | Sub2-2(7)-3 | m/z = 476.06(C₂₈H₁₈BrN₃ = 477.35) |
| Sub2-2(8)-3 | m/z = 476.06(C₂₈H₁₈BrN₃ = 477.35) | Sub2-2(9)-3 | m/z = 476.06(C₂₈H₁₈BrN₃ = 477.35) |
| Sub2-2(10)-3 | m/z = 476.06(C₂₈H₁₈BrN₅ = 477.35) | Sub2-2(11)-3 | m/z = 477.06(C₂₆H₁₆BrN₅ = 478.34) |
| Sub2-2(12)-3 | m/z = 553.09(C₃₂H₂₀BrN₅ = 554.44) | Sub2-2(13)-3 | m/z = 552.09(C₃₃H₂₁BrN₄ = 553.45) |
| Sub2-2(14)-3 | m/z = 552.09(C₃₃H₂₁BrN₄ = 553.45) | Sub2-2(15)-3 | m/z = 551.10(C₃₄H₂₂BrN₃ = 552.46) |
| Sub2-2(16)-3 | m/z = 551.10(C₃₄H₂2BrN₃ = 552.46) | Sub2-2(17)-3 | m/z = 552.09(C₃₃H₂₁BrN₄ = 553.45) |
| Sub2-2(18)-3 | m/z = 553.09(C₃₂H₂₀BrN₅ = 554.44) | Sub2-2(19)-3 | m/z = 553.09(C₃₂H₂₀BrN₅ = 554.44) |
| Sub2-2(20)-3 | m/z = 552.09(C₃₃H₂₂BrN₄ = 553.45) | Sub2-2(21)-3 | m/z = 552.09(C₃₃H₂₁BrN₄ = 553.45) |
| Sub2-2(22)-3 | m/z = 551.10(C₃₄H₂₂BrN₃ = 552.46) | Sub2-2(23)-3 | m/z = 551.10(C₃₄H₂₂BrN₃ = 552.46) |
| Sub2-2(24)-3 | m/z = 552.09(C₃₃H₂₁BrN₄ = 553.45) | Sub2-2(25)-3 | m/z = 552.09(C₃₃H₂₁BrN₄ = 553.45) |
| Sub2-2(26)-3 | m/z = 450.05(C₂₅H₁₅BrN₄ = 451.32) | Sub2-2(27)-3 | m/z = 322.01(C₁₇H₁₁BrN₂ = 323.19) |
| Sub2-2(28)-3 | m/z = 322.01(C₁₇H₁₇BrN₂ = 323.19) | Sub2-2(29)-3 | m/z = 322.01(C₁₇H₁₁BrN₂ = 323.19) |
| Sub2-2(30)-3 | m/z = 398.04(C₃₃H₁₅BrN₂ = 399.28) | Sub2-2(31)-3 | m/z = 398.04(C₂₃H₁₅BrN₂ = 399.28) |
| Sub2-2(32)-3 | m/z = 477.06(C₂₆H₁₆BrN₅ = 478.34) | Sub2-2(33)-3 | m/z = 476.06(C₂₈H₁₈BrN₃ = 477.35) |
| Sub2-2(33)-3 | m/z = 476.06(C₂₈H₁₈BrN₃ = 477.35) | Sub2-2(35)-3 | m/z = 476.06(C₂₈H₁₈BrN₃ = 477.35) |
| Sub2-2(36)-3 | m/z = 475.07(C₂₈H₁₈BrN₃ = 476.37) | Sub2-2(37)-3 | m/z = 475.07(C₂₈H₁₈BrN₃ = 476.37) |
| Sub2-2(38)-3 | m/z = 476.06(C₂₈H₁₈BrN₃ = 477.35) | Sub2-2(39)-3 | m/z = 476.06(C₂₈H₁₈BrN₃ = 477.35) |
| Sub2-2(40)-3 | m/z = 553.09(C₃₂H₂₀BrN₅ = 554.44) | Sub2-2(41)-3 | m/z = 552.09(C₃₃H₂₁BrN₄ = 553.45) |
| Sub2-2(42)-3 | m/z = 552.09(C₃₃H₂₁BrN₄ = 553.45) | Sub2-2(43)-3 | m/z = 551.10(C₃₄H₂₂BrN₃ = 552.46) |
| Sub2-2(44)-3 | m/z = 551.10(C₃₄H₂₂BrN₃ = 552.46) | Sub2-2(45)-3 | m/z = 552.09(C₃₃H₂₁BrN₄ = 553.45) |
| Sub2-2(46)-3 | m/z = 553.09(C₃₂H₃₀BrN₅ = 554.44) | Sub2-2(47)-3 | m/z = 553.09(C₃₂H₂₀BrN₅ = 554.44) |
| Sub2-2(48)-3 | m/z = 552.09(C₃₃H₂₁BrN₄ = 553.45) | Sub2-2(49)-3 | m/z = 552.09(C₃₃H₂₁BrN₄ = 553.45) |
| Sub2-2(50)-3 | m/z = 551.10(C₃₄H₂₂BrN₃ = 552.46) | Sub2-2(51)-3 | m/z = 551.10(C₃₄H₂₂BrN₃ = 552.46) |
| Sub2-2(52)-3 | m/z = 552.09(C₃₃H₂₂BrN₄ = 553.45) | Sub2-2(53)-3 | m/z = 553.09(C₃₂H₂₀BrN₅ = 554.44) |
| Sub2-2(54)-3 | m/z = 450.04(C₂₅H₁₅BrN₄ = 451.32) | Sub2-2(55)-3 | m/z = 322.01(C₁₇H₁₁BrN₂ = 323.19) |
| Sub2-2(56)-3 | m/z = 322.01(C₁₇H₁₁BrN₂ = 323.19) | Sub2-2(57)-3 | m/z = 322.01(C₁₇H₁₁BrN₂ = 323.19) |
| Sub2-3(1)-3 | m/z = 322.01(C₁₇H₁₁BrN₂ = 323.19) | Sub2-3(2)-3 | m/z = 398.04(C₂₃H₁₅BrN₂ = 399.28) |
| Sub2-3(3)-3 | m/z = 398.04(C₂₃H₁₅BrN₂ = 389.28) | Sub2-3(4)-3 | m/z = 477.06(C₂₆H₁₆BrN₅ = 478.34) |
| Sub2-3(5)-3 | m/z = 475.07(C₂₈H₁₈BrN₃ = 476.37) | Sub2-3(6)-3 | m/z = 475.07(C₂₈H₁₈BrN₃ = 476.37) |
| Sub2-3(7)-3 | m/z = 476.06(C₂₈H₁₈BrN₃ = 477.35) | Sub2-3(8)-3 | m/z = 476.06(C₂₈H₁₈BrN₃ = 477.35) |
| Sub2-3(9)-3 | m/z = 476.06(C₂₈H₁₈BrN₃ = 477.35) | Sub2-3(10)-3 | m/z = 476.06(C₂₈H₁₈BrN₃ = 477.35) |
| Sub2-3(11)-3 | m/z = 477.06(C₂₈H₁₈BrN₃ = 478.34) | Sub2-3(12)-3 | m/z = 551.10(C₃₄H₂₂BrN₃ = 552.46) |
| Sub2-3(13)-3 | m/z = 551.10(C₃₄H₂₂BrN₃ = 552.46) | Sub2-3(14)-3 | m/z = 552.09(C₃₃H₂₁BrN₄ = 553.45) |
| Sub2-3(15)-3 | m/z = 553.09(C₃₂H₂₀BrN₅ = 554.44) | Sub2-3(16)-3 | m/z = 553.09(C₃₂H₂₀BrN₅ = 554.44) |
| Sub2-3(17)-3 | m/z = 552.09(C₃₃H₂₁BrN₄ = 553.45) | Sub2-3(18)-3 | m/z = 552.09(C₃₃H₂₁BrN₄ = 553.45) |
| Sub2-3(19)-3 | m/z = 553.09(C₃₂H₂₀BrN₅ = 554.44) | Sub2-3(20)-3 | m/z = 552.09(C₃₃H₂₁BrN₄ = 553.45) |
| Sub2-3(21)-3 | m/z = 552.09(C₃₃H₂₁BrN₄ = 553.45) | Sub2-3(22)-3 | m/z = 551.10(C₃₄H₂₇BrN₃ = 552.46) |
| Sub2-3(23)-3 | m/z = 551.10(C₃₄H₂₇BrN₃ = 552.46) | Sub2-3(24)-3 | m/z = 552.09(C₃₃H₂₁BrN₄ = 553.45) |
| Sub2-3(25)-3 | m/z = 553.09(C₃₂H₂₀BrN₅ = 554.44) | Sub2-3(26)-3 | m/z = 450.05(C₂₅H₁₅BrN₄ = 451.32) |
| Sub2-3(27)-3 | m/z = 322.01(C₁₇H₁₁BrN₂ = 323.19) | Sub2-3(28)-3 | m/z = 322.01(C₁₇H₁₁BrN₂ = 323.19) |
| Sub2-3(29)-3 | m/z = 322.01(C₁₇H₁₁BrN₂ = 323.19) | Sub2-3(30)-3 | m/z = 398.04(C₂₈H₁₅BrN₂ = 399.28) |
| Sub2-3(31)-3 | m/z = 398.04(C₂₈H₁₅BrN₂ = 399.28) | Sub2-3(32)-3 | m/z = 450.05(C₂₅H₁₅BrN₄ = 451.32) |
| Sub2-3(33)-3 | m/z = 475.07(C₂₈H₁₈BrN₃ = 476.37) | Sub2-3(34)-3 | m/z = 475.07(C₂₈H₁₈BrN₃ = 476.37) |
| Sub2-3(35)-3 | m/z = 476.06(C₂₈H₁₈BrN₃ = 477.35) | Sub2-3(36)-3 | m/z = 476.06(C₂₈H₁₈BrN₃ = 477.35) |
| Sub2-3(37)-3 | m/z = 476.06(C₂₈H₁₈BrN₃ = 477.35) | Sub2-3(38)-3 | m/z = 476.06(C₂₈H₁₈BrN₃ = 477.35) |
| Sub2-3(39)-3 | m/z = 477.06(C₂₆H₁₈BrN₅ = 478.34) | Sub2-3(40)-3 | m/z = 477.06(C₂₆H₁₈BrN₅ = 478.34) |
| Sub2-3(41)-3 | m/z = 551.10(C₃₄H₂₂BrN₃ = 552.46) | Sub2-3(42)-3 | m/z = 551.10(C₃₄H₂₂BrN₃ = 552.46) |
| Sub2-3(43)-3 | m/z = 552.09(C₃₃H₂₁BrN₄ = 553.45) | Sub2-3(44)-3 | m/z = 553.09(C₃₂H₂₀BrN₅ = 554.44) |
| Sub2-3(45)-3 | m/z = 553.09(C₃₂H₂₀BrN₅ = 554.44) | Sub2-3(46)-3 | m/z = 552.09(C₃₃H₂₁BrN₄ = 553.45) |
| Sub2-3(47)-3 | m/z = 552.09(C₃₃H₂₁BrN₄ = 553.45) | Sub2-3(48)-3 | m/z = 553.09(C₃₂H₂₀BrN₅ = 554.44) |
| Sub2-3(49)-3 | m/z = 551.10(C34H₂₂BrN₃ = 552.46) | Sub2-3(50)-3 | m/z = 551.10(C₃₄H₂₂BrN₃ = 552.46) |
| Sub2-3(51)-3 | m/z = 552.09(C₃₃H₂₁BrN₄ = 553.45) | Sub2-3(52)-3 | m/z = 553.09(C₃₂H₂₀BrN₅ = 554.44) |
| Sub2-3(53)-3 | m/z = 553.09(C₃₂H₂₀BrN₅ = 554.44) | Sub2-3(54)-3 | m/z = 552.09(C₃₃H₂₁BrN₄ = 553.45) |
| Sub2-3(55)-3 | m/z = 553.09(C₃₂H₂₀BrN₃ = 554.44) | Sub2-4(1)-3 | m/z = 322.01(C₁₇H₁₁BrN₂ = 323.19) |
| Sub2-4(2)-3 | m/z = 322.01(C₁₇H₁₁BrN₂ = 323.19) | Sub2-4(3)-3 | m/z = 322.01(C₁₇H₁₁BrN₂ = 323.19) |
| Sub2-4(4)-3 | m/z = 322.01(C₁₇H₁₁BrN₂ = 323.19) | Sub2-4(5)-3 | m/z = 322.01(C₁₇H₁₁BrN₂ = 323.19) |
| Sub2-4(6)-3 | m/z = 322.01(C₁₇H₁₁BrN₂ = 323.19) | Sub2-4(7)-3 | m/z = 322.01(C₁₇H₁₁BrN₂ = 323.19) |

### □. Synthesis Example of Final Products

In a round-bottom flask, compound Sub 1-3 (1 eq) was added, and then compound Sub 2-3 (1.1 eq), Pd(PPh₃)₄ (0.03-0.05 eq.), NaOH (3 eq), THF (3 mL/1 mmol), and water (1.5 mL/1 mmol) were added. Thereafter, the mixture was heated under reflux at 80-90□. Upon completion of the reaction, the reaction product was diluted with distilled water at room temperature, followed by extraction with methylene chloride and water. The organic layer was dried over MgSO₄ and concentrated, and then the generated compound was subjected to silica gel chromatography and recrystallization to give a product.

### Synthesis Example of Compound 1-1-3

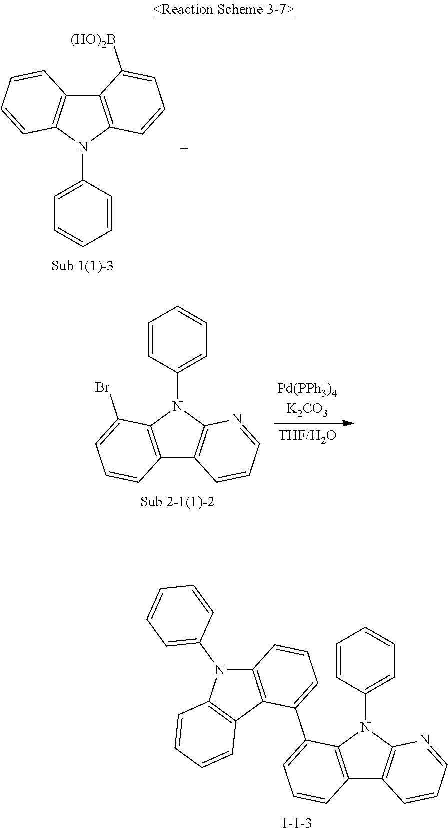 [SMILES: OB(O)c1cccc2c1c1ccccc1n2-c1ccccc1 ; Brc1cccc2c3cccnc3n(-c3ccccc3)c12 ; c1ccc(-n2c3ccccc3c3c(-c4cccc5c6cccnc6n(-c6ccccc6)c45)cccc32)cc1]

In a round-bottom flask, (9-phenyl-9H-carbazol-4-yl)boronic acid (5.7 g, 20 mmol) was added, and 8-bromo-9-phenyl-9H-pyrido[2,3-b]indole (12.2 g, 22 mmol), Pd(PPh₃)₄ (0.5 g, 0.6 mmol), K₂CO₃ (8.3 g, 60 mmol), THF (60 mL), and water (30 mL) were added. Thereafter, the mixture was heated under reflux at 80-90□. Upon completion of the reaction, the reaction product was diluted with distilled water at room temperature, followed by extraction with methylene chloride and water. The organic layer was dried over MgSO₄ and concentrated, and then the thus generated compound was subjected to silica gel column chromatography and recrystallization to give a product 5.6 g (yield: 58%).

### 2. Synthesis Example of Compound 2-38-3

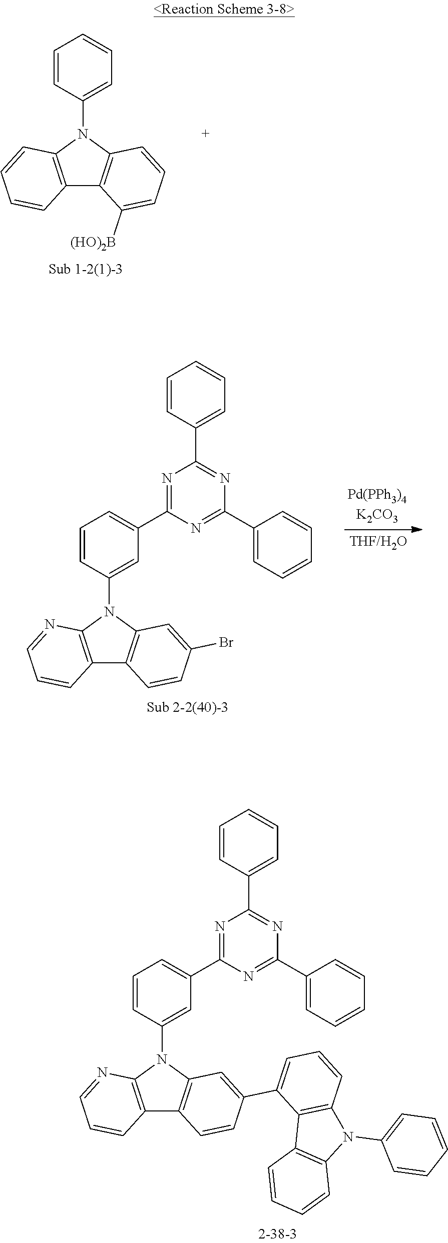 [SMILES: OB(O)c1cccc2c1c1ccccc1n2-c1ccccc1 ; Brc1ccc2c3cccnc3n(-c3cccc(-c4nc(-c5ccccc5)nc(-c5ccccc5)n4)c3)c2c1 ; c1ccc(-c2nc(-c3ccccc3)nc(-c3cccc(-n4c5cc(-c6cccc7c6c6ccccc6n7-c6ccccc6)ccc5c5cccnc54)c3)n2)cc1]

In a round-bottom flask, (9-phenyl-9H-carbazol-4-yl)boronic acid (5.7 g, 20 mmol) was added, and 7-bromo-9-(3-(4,6-diphenyl-1,3,5-triazin-2-yl)phenyl)-9H-pyrido[2,3-b]indole (12.2 g, 22 mmol), Pd(PPh₃)₄ (0.03-0.05 eq), K₂CO₃ (3 eq), THF (10 mL), and water (5 mL) were added. Thereafter, the mixture was heated under reflux at 80-90□. Upon completion of the reaction, the reaction product was diluted with distilled water at room temperature, followed by extraction with methylene chloride and water. The organic layer was dried over MgSO₄ and concentrated, and then the thus generated compound was subjected to silica gel column chromatography and recrystallization to give a product 8.2 g (yield: 57%).

### 3. Synthesis Example of Compound 2-70-3

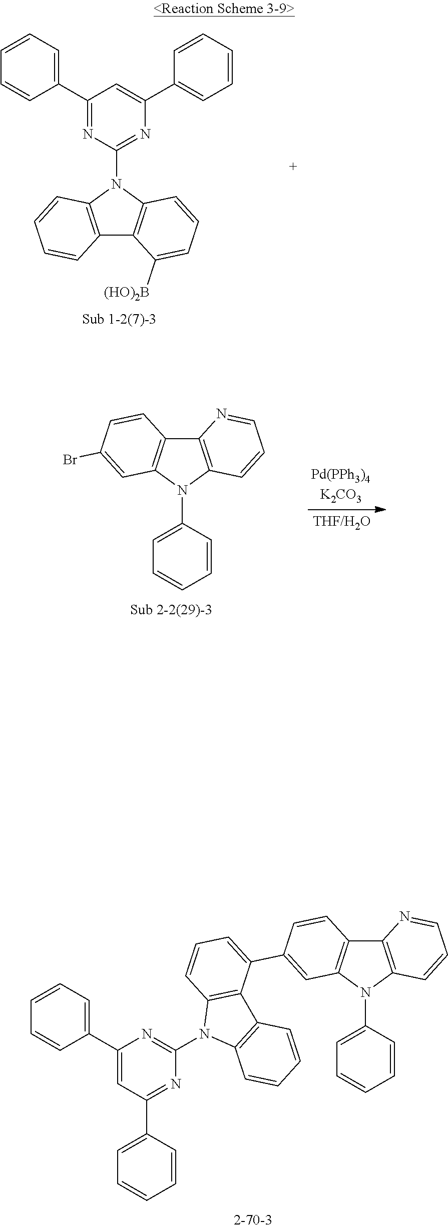 [SMILES: OB(O)c1cccc2c1c1ccccc1n2-c1nc(-c2ccccc2)cc(-c2ccccc2)n1 ; Brc1ccc2c3ncccc3n(-c3ccccc3)c2c1 ; c1ccc(-c2cc(-c3ccccc3)nc(-n3c4ccccc4c4c(-c5ccc6c7ncccc7n(-c7ccccc7)c6c5)cccc43)n2)cc1]

In a round-bottom flask, (9-(4,6-diphenylpyrimidin-2-yl)-9H-carbazol-4-yl)boronic acid (8.8 g, 20 mmol) was added, and then, 7-bromo-9-(3-(4,6-diphenyl-1,3,5-triazin-2-yl)phenyl)-9H-pyrido[2,3-b]indole (12.2 g, 22 mmol), Pd(PPh₃)₄ (0.03-0.05 eq), K₂CO₃ (3 eq), THF (10 mL), and water (5 mL) were added. Thereafter, the mixture was heated under reflux at 80-90° C. Upon completion of the reaction, the reaction product was diluted with distilled water at room temperature, followed by extraction with methylene chloride and water. The organic layer was dried over MgSO₄ and concentrated, and then the thus generated compound was subjected to silica gel column chromatography and recrystallization to give a product 8.0 g (yield: 62%).

### 4. Synthesis Example of Compound 3-10-3

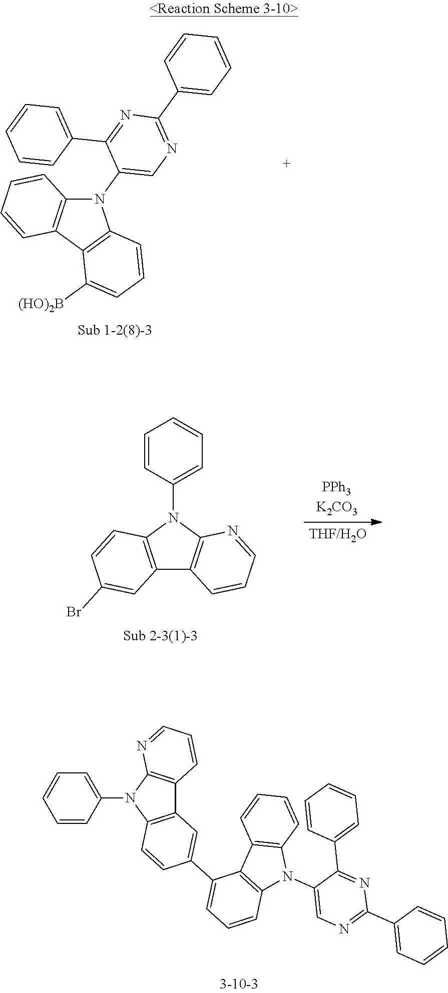 [SMILES: OB(O)c1cccc2c1c1ccccc1n2-c1cnc(-c2ccccc2)nc1-c1ccccc1 ; Brc1ccc2c(c1)c1cccnc1n2-c1ccccc1 ; c1ccc(-c2ncc(-n3c4ccccc4c4c(-c5ccc6c(c5)c5cccnc5n6-c5ccccc5)cccc43)c(-c3ccccc3)n2)cc1]

In a round-bottom flask, (9-(2,4-diphenylpyrimidin-5-yl)-9H-carbazol-1-yl)boronic acid (8.8 g, 20 mmol) was added, and then 6-bromo-9-phenyl-9H-pyrido[2,3-b]indole (7.1 g, 22 mmol), Pd(PPh₃)₄ (0.03-0.05 eq), K₂CO₃ (3 eq), THF (10 mL), and water (5 mL) were added. Thereafter, the mixture was heated under reflux at 80-90° C. Upon completion of the reaction, the reaction product was diluted with distilled water at room temperature, followed by extraction with methylene chloride and water. The organic layer was dried over MgSO₄ and concentrated, and then the thus generated compound was subjected to silica gel column chromatography and recrystallization to give a product 7.3 g (yield: 57%).

### 5. Synthesis Example of Compound 3-68-3

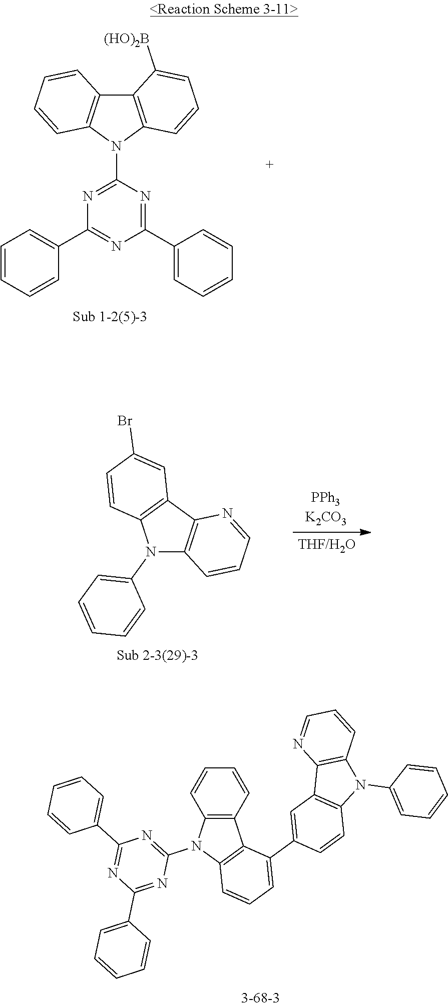 [SMILES: OB(O)c1cccc2c1c1ccccc1n2-c1nc(-c2ccccc2)nc(-c2ccccc2)n1 ; Brc1ccc2c(c1)c1ncccc1n2-c1ccccc1 ; c1ccc(-c2nc(-c3ccccc3)nc(-n3c4ccccc4c4c(-c5ccc6c(c5)c5ncccc5n6-c5ccccc5)cccc43)n2)cc1]

In a round-bottom flask, (9-(4,6-diphenyl-1,3,5-triazin-2-yl)-9H-carbazol-4-yl)boronic acid (8.8 g, 20 mmol) was added, and then 8-bromo-5-phenyl-5H-pyrido[3,2-b]indole (7.1 g, 22 mmol), Pd(PPh₃)₄ (0.03-0.05 eq), K₂CO₃ (3 eq), THF (10 mL), and water (5 mL) were added. Thereafter, the mixture was heated under reflux at 80-90° C. Upon completion of the reaction, the reaction product was diluted with distilled water at room temperature, followed by extraction with methylene chloride and water. The organic layer was dried over MgSO₄ and concentrated, and then the thus generated compound was subjected to silica gel column chromatography and recrystallization to give a product 7.0 g (yield: 54%).

### 6. Synthesis Example of Compound 3-76-3

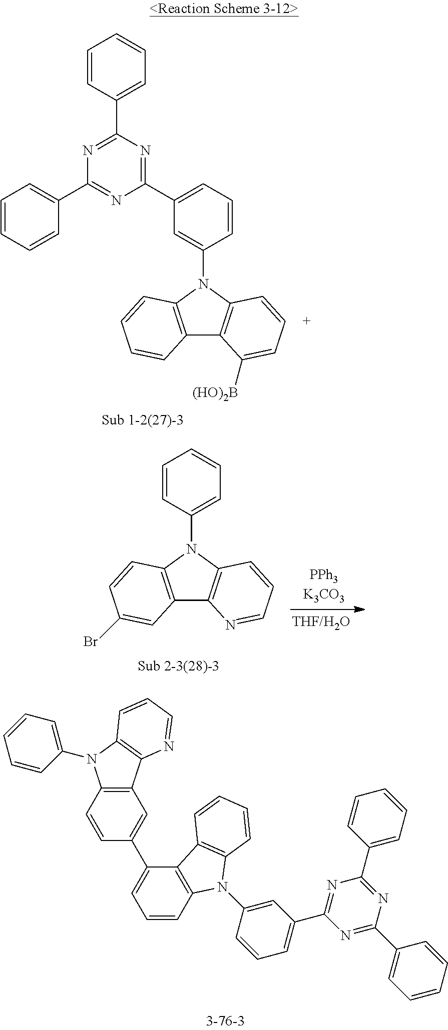 [SMILES: OB(O)c1cccc2c1c1ccccc1n2-c1cccc(-c2nc(-c3ccccc3)nc(-c3ccccc3)n2)c1 ; Brc1ccc2c(c1)c1ncccc1n2-c1ccccc1 ; c1ccc(-c2nc(-c3ccccc3)nc(-c3cccc(-n4c5ccccc5c5c(-c6ccc7c(c6)c6ncccc6n7-c6ccccc6)cccc54)c3)n2)cc1]

In a round-bottom flask, (9-(3-(4,6-diphenyl-1,3,5-triazin-2-yl)phenyl)-9H-carbazol-4-yl)boronic acid (10.4 g, 20 mmol) was added, and then 8-bromo-5-phenyl-5H-pyrido[3,2-b]indole (7.1 g, 22 mmol), Pd(PPh₃)₄ (0.03-0.05 eq), K₂CO₃ (3 eq), THF (10 mL), and water (5 mL) were added. Thereafter, the mixture was heated under reflux at 80-90° C. Upon completion of the reaction, the reaction product was diluted with distilled water at room temperature, followed by extraction with methylene chloride and water. The organic layer was dried over MgSO₄ and concentrated, and then the thus generated compound was subjected to silica gel column chromatography and recrystallization to give a product 10.5 g (yield: 73%).

### 7. Synthesis Example of Compound 4-23-3

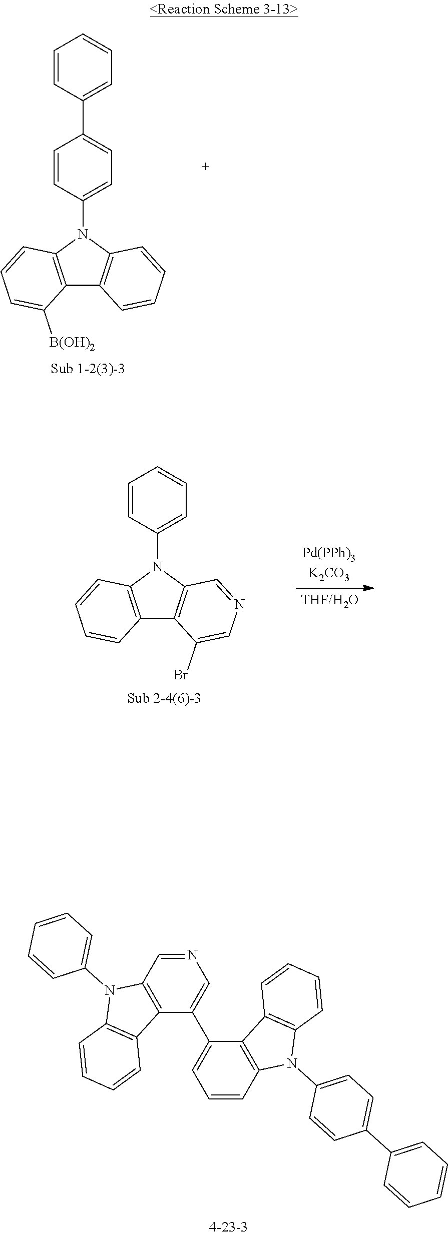 [SMILES: OB(O)c1cccc2c1c1ccccc1n2-c1ccc(-c2ccccc2)cc1 ; Brc1cncc2c1c1ccccc1n2-c1ccccc1 ; c1ccc(-c2ccc(-n3c4ccccc4c4c(-c5cncc6c5c5ccccc5n6-c5ccccc5)cccc43)cc2)cc1]

In a round-bottom flask, (9-([1,1′-biphenyl]-4-yl)-9H-carbazol-4-yl)boronic acid (7.2 g, 20 mmol) was added, 4-bromo-9-phenyl-9H-pyrido[3,4-b]indole (7.1 g, 22 mmol), Pd(PPh₃)₄ (0.03-0.05 eq), K₂CO₃ (3 eq), THF (10 mL), and water (5 mL) were added. Thereafter, the mixture was heated under reflux at 80-90° C. Upon completion of the reaction, the reaction product was diluted with distilled water at room temperature, followed by extraction with methylene chloride and water. The organic layer was dried over MgSO₄ and concentrated, and then the thus generated compound was subjected to silica gel column chromatography and recrystallization to give a product 7.8 g (yield: 69%).

Meanwhile, FD-MS values of compounds 1-1-3 to 1-28-3, 2-1-3 to 2-128-3, 3-1-3 to 3-128-3, 4-1-3 to 4-28-3, and 5-1-3 to 5-4-3 of the present invention prepared by the above synthesis examples are shown as in table 3-4 below.

TABLE 3-4

| Compound | FD-MS | Compound | FD-MS |
|---|---|---|---|
| 1-1-3 | m/z = 485.19 (C₃₅H₂₃N₃ = 485.58) | 1-2-3 | m/z = 535.20 (C₃₉H₂₅N₃ = 535.64) |
| 1-3-3 | m/z = 561.22 (C₄₁H₂₇N₃ = 561.67) | 1-4-3 | m/z = 640.24 (C₄₄H₂₈N₆ = 640.73) |
| 1-5-3 | m/z = 485.19 (C₃₅H₂₃N₃ = 485.58) | 1-6-3 | m/z = 535.20 (C₃₉H₂₅N₃ = 535.64) |
| 1-7-3 | m/z = 561.22 (C₄₁H₂₇N₃ = 561.67) | 1-8-3 | m/z = 640.24 (C₄₄H₂₈N₆ = 640.73) |
| 1-9-3 | m/z = 485.19 (C₃₅H₂₃N₃ = 485.58) | 1-10-3 | m/z = 535.20 (C₃₉H₂₅N₃ = 535.64) |
| 1-11-3 | m/z = 561.22 (C₄₁H₂₇N₃ = 561.67) | 1-12-3 | m/z = 640.24 (C₄₄H₂₈N₆ = 640.73) |
| 1-13-3 | m/z = 485.19 (C₃₅H₂₃N₃ = 485.5.8) | 1-14-3 | m/z = 535.20 (C₃₉H₂₅N₃ = 535.64) |
| 1-15-3 | m/z = 561.22 (C₄₁H₂₂N₃ = 561.67) | 1-16-3 | m/z = 640.24 (C₄₄H₂₈N₆ = 640.73) |
| 1-17-3 | m/z = 485.19 (C₃₅H₂₃N₃ = 485.58) | 1-18-3 | m/z = 535.20 (C₃₉H₂₅N₃ = 535.64) |
| 1-19-3 | m/z = 561.22 (C₄₁H₂₇N₃ = 561.67) | 1-20-3 | m/z = 640.24 (C₄₄H₂₈N₆ = 640.73) |
| 1-21-3 | m/z = 485.19 (C₃₅H₂₃N₃ = 485.58) | 1-22-3 | m/z = 535.20 (C₃₉H₂₅N₃ = 535.64) |
| 1-23-3 | m/z = 561.22 (C₄₁H₂₇N₃ = 561.67) | 1-24-3 | m/z = 640.24 (C₄₄H₂₈N₆ = 640.73) |
| 1-25-3 | m/z = 485.19 (C₃₅H₂₃N₃ = 485.58) | 1-26-3 | m/z = 535.20 (C₃₉H₂₅N₃ = 535.64) |
| 1-27-3 | m/z = 561.22 (C₄₁H₂₇N₃ = 561.67) | 1-28-3 | m/z = 640.24 (C₄₄H₂₈N₆ = 640.73) |
| 2-1-3 | m/z = 485.19 (C₃₅H₂₃N₃ = 485.5.8) | 2-2-3 | m/z = 561.22 (C₄₁H₂₇N₃ = 561.67) |
| 2-3-3 | m/z = 561.22 (C₄₁H₂₂N₃ = 561.67) | 2-4-3 | m/z = 637.25 (C₄₇H₃₁N₃ = 637.77) |
| 2-5-3 | m/z = 637.25 (C₄₇H₃₁N₃ = 637.77) | 2-6-3 | m/z = 637.25 (C₄₇H₃₁N₃ = 637.77) |
| 2-7-3 | m/z = 637.25 (C₄₇H₃₁N₃ = 637.77) | 2-8-3 | m/z = 639.24 (C₄₅H₂₉N₅ = 639.75) |
| 2-9-3 | m/z = 639.24 (C₄₅H₂₉N₅ = 639.75) | 2-10-3 | m/z = 639.24 (C₄₅H₂₉N₅ = 639.75) |
| 2-11-3 | m/z = 638.25 (C₄₆H₃₀N₄ = 638.76) | 2-12-3 | m/z = 638.25 (C₄₆H₃₀N₄ = 638.76) |
| 2-13-3 | m/z = 639.24 (C₄₅H₂₉N₅ = 639.75) | 2-14-3 | m/z = 640.24 (C₄₄H₂₈N₆ = 640.73) |
| 2-15-3 | m/z = 716.27 (C₃₈H₃₂N₆ = 716.83) | 2-16-3 | m/z = 715.27 (C₅₁H₃₃N₅ = 715.84) |
| 2-17-3 | m/z = 715.27 (C₅₁H₃₃N₅ = 715.84) | 2-18-3 | m/z = 714.28 (C₅₂H₃₄N₄ = 714.85) |
| 2-19-3 | m/z = 714.28 (C₅₂H₃₄N₄ = 714.85) | 2-20-3 | m/z = 715.27 (C₅₁H₃₃N₅ = 715.84) |
| 2-21-3 | m/z = 716.27 (C₅₀H₃₂N₆ = 716.83) | 2-22-3 | m/z = 716.27 (C₃₈H₃₂N₆ = 716.83) |
| 2-23-3 | m/z = 715.27 (C₅₁H₃₃N₅ = 715.84) | 2-24-3 | m/z = 715.27 (C₅₁H₃₃N₅ = 715.84) |
| 2-25-3 | m/z = 714.28 (C₅₂H₃₄N₄ = 714.85) | 2-26-3 | m/z = 714.28 (C₅₂H₃₄N₄ = 714.85) |
| 2-27-3 | m/z = 715.27 (C₅₁H₃₃N₅ = 715.84) | 2-28-3 | m/z = 716.27 (C₃₈H₃₂N₆ = 716.83) |
| 2-29-3 | m/z = 613.23 (C₄₃H₂₇N₅ = 613.71) | 2-30-3 | m/z = 640.24 (C₄₄H₂₈N₆ = 640.73) |
| 2-31-3 | m/z = 639.24 (C₄₅H₂₉N₅ = 639.75) | 2-32-3 | m/z = 639.24 (C₄₅H₂₉N₅ = 639.75) |
| 2-33-3 | m/z = 639.24 (C₄₅H₂₉N₅ = 639.75) | 2-34-3 | m/z = 638.25 (C₄₆H₃₀N₄ = 638.76) |
| 2-35-3 | m/z = 638.25 (C₄₆H₃₀N₄ = 638.76) | 2-36-3 | m/z = 639.24 (C₄₅H₂₉N₅ = 639.75) |
| 2-37-3 | m/z = 640.24 (C₄₄H₂₈N₆ = 640.73) | 2-38-3 | m/z = 716.27 (C₃₈H₃₂N₆ = 716.83) |
| 2-39-3 | m/z = 715.27 (C₅₁H₃₃N₅ = 715.84) | 2-40-3 | m/z = 715.27 (C₅₁H₃₃N₅ = 715.84) |
| 2-41-3 | m/z = 714.28 (C₅₂H₃₄N₄ = 714.85) | 2-42-3 | m/z = 714.28 (C₅₂H₃₄N₄ = 714.85) |
| 2-43-3 | m/z = 715.27 (C₅₁H₃₃N₅ = 715.84) | 2-44-3 | m/z = 716.27 (C₅₀H₃₂N₆ = 716.83) |
| 2-45-3 | m/z = 716.27 (C₅₀H₃₂N₆ = 716.83) | 2-46-3 | m/z = 715.27 (C₅₁H₃₃N₅ = 715.84) |
| 2-47-3 | m/z = 715.27 (C₅₁H₃₃N₅ = 715.84) | 2-48-3 | m/z = 714.28 (C₅₂H₃₄N₄ = 714.85) |
| 2-49-3 | m/z = 714.28 (C₅₂H₃₄N₄ = 714.85) | 2-50-3 | m/z = 715.27 (C₅₁H₃₃N₅ = 715.84) |
| 2-51-3 | m/z = 716.27 (C₅₀H₃₂N₆ = 716.83) | 2-52-3 | m/z = 613.23 (C₄₃H₂₇N₅ = 613.71) |
| 2-53-3 | m/z = 485.19 (C₃₅H₂₃N₃ = 485.58) | 2-54-3 | m/z = 535.20 (C₃₉H₂₅N₃ = 535.64) |
| 2-55-3 | m/z = 561.22 (C₄₁H₂₂N₃ = 561.67) | 2-56-3 | m/z = 640.24 (C₄₄H₂₈N₆ = 640.73) |
| 2-57-3 | m/z = 485.19 (C₃₅H₂₃N₃ = 485.58) | 2-58-3 | m/z = 535.20 (C₃₉H₂₅N₃ = 535.64) |
| 2-59-3 | m/z = 561.22 (C₄₁H₂₂N₃ = 561.67) | 2-60-3 | m/z = 640.24 (C₄₄H₂₈N₆ = 640.73) |
| 2-61-3 | m/z = 485.19 (C₃₅H₂₃N₃ = 485.58) | 2-62-3 | m/z = 561.22 (C₄₁H₂₂N₃ = 561.67) |
| 2-63-3 | m/z = 561.22 (C₄₁H₂₂N₃ = 561.67) | 2-64-3 | m/z = 637.25 (C₄₇H₃₁N₃ = 637.77) |
| 2-65-3 | m/z = 637.25 (C₄₇H₃₁N₃ = 637.77) | 2-66-3 | m/z = 637.25 (C₄₇H₃₁N₃ = 637.77) |
| 2-67-3 | m/z = 637.25 (C₄₇H₃₁N₃ = 637.77) | 2-68-3 | m/z = 640.24 (C₄₄H₂₈N₆ = 640.73) |
| 2-69-3 | m/z = 639.24 (C₄₅H₂₉N₅ = 639.75) | 2-70-3 | m/z = 639.24 (C₄₅H₂₉N₅ = 639.75) |
| 2-71-3 | m/z = 639.24 (C₄₅H₂₉N₅ = 639.75) | 2-72-3 | m/z = 638.25 (C₄₆H₃₀N₄ = 638.76) |
| 2-73-3 | m/z = 638.25 (C₄₆H₃₀N₄ = 638.76) | 2-74-3 | m/z = 639.24 (C₄₅H₂₉N₅ = 639.75) |
| 2-75-3 | m/z = 640.24 (C₄₄H₂₈N₆ = 640.73) | 2-76-3 | m/z = 716.27 (C₅₀H₃₂N₆ = 716.83) |
| 2-77-3 | m/z = 715.27 (C₅₁H₃₃N₅ = 715.84) | 2-78-3 | m/z = 715.27 (C₅₁H₃₃N₅ = 715.84) |
| 2-79-3 | m/z = 714.28 (C₅₂H₃₄N₄ = 714.85) | 2-80-3 | m/z = 714.28 (C₅₂H₃₄N₄ = 714.85) |
| 2-81-3 | m/z = 715.27 (C₅₁H₃₃N₅ = 715.84) | 2-82-3 | m/z = 716.27 (C₅₀H₃₂N₆ = 716.83) |
| 2-83-3 | m/z = 716.27 (C₅₀H₃₂N₆ = 716.83) | 2-84-3 | m/z = 715.27 (C₅₁H₃₃N₅ = 715.84) |
| 2-85-3 | m/z = 715.27 (C₅₁H₃₃N₅ = 715.84) | 2-86-3 | m/z = 714.28 (C₅₂H₃₄N₄ = 714.85) |
| 2-87-3 | m/z = 714.28 (C₅₂H₃₄N₄ = 714.85) | 2-88-3 | m/z = 715.27 (C₅₁H₃₃N₅ = 715.84) |
| 2-89-3 | m/z = 716.27 (C₅₀H₃₂N₆ = 716.83) | 2-90-3 | m/z = 613.23 (C₄₃H₂₇N₅ = 613.71) |
| 2-91-3 | m/z = 640.24 (C₄₄H₂₈N₆ = 640.73) | 2-92-3 | m/z = 639.24 (C₄₅H₂₉N₅ = 639.75) |
| 2-93-3 | m/z = 639.24 (C₄₅H₂₉N₅ = 639.75) | 2-94-3 | m/z = 639.24 (C₄₅H₂₉N₅ = 639.75) |
| 2-95-3 | m/z = 638.25 (C₄₆H₃₀N₄ = 638.76) | 2-96-3 | m/z = 638.25 (C₄₆H₃₀N₄ = 638.76) |
| 2-97-3 | m/z = 639.24 (C₄₅H₂₉N₅ = 639.75) | 2-98-3 | m/z = 640.24 (C₄₄H₂₈N₆ = 640.73) |
| 2-99-3 | m/z = 716.27 (C₅₀H₃₂N₆ = 716.83) | 2-100-3 | m/z = 715.27 (C₅₁H₃₃N₅ = 715.84) |
| 2-101-3 | m/z = 715.27 (C₅₁H₃₃N₅ = 715.84) | 2-102-3 | m/z = 714.28 (C₅₂H₃₄N₄ = 714.85) |
| 2-103-3 | m/z = 714.28 (C₅₂H₃₄N₄ = 714.85) | 2-104-3 | m/z = 715.27 (C₅₁H₃₃N₅ = 715.84) |
| 2-105-3 | m/z = 716.27 (C₅₀H₃₂N₆ = 716.83) | 2-106-3 | m/z = 716.27 (C₅₀H₃₂N₆ = 716.83 |
| 2-107-3 | m/z = 715.27 (C₅₁H₃₃N₅ = 715.84) | 2-108-3 | m/z = 715.27 (C₅₁H₃₃N₅ = 715.84) |
| 2-109-3 | m/z = 714.28 (C₅₂H₃₄N₄ = 714.85) | 2-110-3 | m/z = 714.28 (C₅₂H₃₄N₄ = 714.85) |
| 2-111-3 | m/z = 715.27 (C₅₁H₃₃N₅ = 715.84) | 2-112-3 | m/z = 716.27 (C₅₀H₃₂N₆ = 716.83) |
| 2-113-3 | m/z = 613.23 (C₄₃H₂₇N₅ = 613.71) | 2-114-3 | m/z = 485.19 (C₃₅H₂₃N₃ = 485.58) |
| 2-115-3 | m/z = 535.20 (C₃₉H₂₅N₃ = 535.64) | 2-116-3 | m/z = 561.22 (C₄₁H₂₂N₃ = 561.67) |
| 2-117-3 | m/z = 640.24 (C₄₄H₂₈N₆ = 640.73) | 2-118-3 | m/z = 485.19 (C₃₅H₂₃N₃ = 485.58) |
| 2-119-3 | m/z = 535.20 (C₃₉H₂₅N₃ = 535.64) | 2-120-3 | m/z = 561.22 (C₄₁H₂₂N₃ = 561.67) |
| 2-121-3 | m/z = 640.24 (C₄₄H₂₈N₆ = 640.73) | 2-122-3 | m/z = 485.19 (C₃₅H₂₃N₃ = 485.58) |
| 2-123-3 | m/z = 535.20 (C₃₉H₂₅N₃ = 535.64) | 2-124-3 | m/z = 561.22 (C₄₁H₂₂N₃ = 561.67) |
| 2-125-3 | m/z = 640.24 (C₄₄H₂₈N₆ = 640.73) | 2-126-3 | m/z = 640.24 (C₄₄H₂₈N₆ = 640.73) |
| 2-127-3 | m/z = 535.20 (C₃₉H₂₅N₃ = 535.64) | 2-128-3 | m/z = 535.20 (C₃₉H₂₅N₃ = 535.64) |
| 3-1-3 | m/z = 485.19 (C₃₅H₂₃N₃ = 485.58) | 3-2-3 | m/z = 561.22 (C₄₁H₂₂N₃ = 561.67) |
| 3-3-3 | m/z = 561.22 (C₄₁H₂₂N₃ = 561.67) | 3-4-3 | m/z = 637.25 (C₄₇H₃₁N₃ = 637.77) |
| 3-5-3 | m/z = 637.25 (C₄₇H₃₁N₃ = 637.77) | 3-6-3 | m/z = 637.25 (C₄₇H₃₁N₃ = 637.77) |
| 3-7-3 | m/z = 637.25 (C₄₇H₃₁N₃ = 637.77) | 3-8-3 | m/z = 639.24 (C₄₅H₂₉N₅ = 639.75) |
| 3-9-3 | m/z = 639.24 (C₄₅H₂₉N₅ = 639.75) | 3-10-3 | m/z = 639.24 (C₄₅H₂₉N₅ = 639.75) |
| 3-11-3 | m/z = 638.25 (C₄₆H₃₀N₄ = 638.76) | 3-12-3 | m/z = 638.25 (C₄₆H₃₀N₄ = 638.76) |
| 3-13-3 | m/z = 639.24 (C₄₅H₂₉N₅ = 639.75) | 3-14-3 | m/z = 640.24 (C₄₄H₂₈N₆ = 640.73) |
| 3-15-3 | m/z = 716.27 (C₅₀H₃₂N₆ = 716.83) | 3-16-3 | m/z = 715.27 (C₅₁H₃₃N₅ = 715.84) |
| 3-17-3 | m/z = 715.27 (C₅₁H₃₃N₅ = 715.84) | 3-18-3 | m/z = 714.28 (C₅₂H₃₄N₄ = 714.85) |
| 3-19-3 | m/z = 714.28 (C₅₂H₃₄N₄ = 714.85) | 3-20-3 | m/z = 715.27 (C₅₁H₃₃N₅ = 715.84) |
| 3-21-3 | m/z = 716.27 (C₅₀H₃₂N₆ = 716.83) | 3-22-3 | m/z = 716.27 (C₅₀H₃₂N₆ = 716.83) |
| 3-23-3 | m/z = 715.27 (C₅₁H₃₃N₅ = 715.84) | 3-24-3 | m/z = 715.27 (C₅₁H₃₃N₅ = 715.84) |
| 3-25-3 | m/z = 714.28 (C₅₂H₃₄N₄ = 714.85) | 3-26-3 | m/z = 714.28 (C₅₂H₃₄N₄ = 714.85) |
| 3-27-3 | m/z = 715.27 (C₅₁H₃₃N₅ = 715.84) | 3-28-3 | m/z = 716.27 (C₅₀H₃₂N₆ = 716.83) |
| 3-29-3 | m/z = 613.23 (C₄₃H₂₇N₅ = 613.71) | 3-30-3 | m/z = 640.24 (C₄₄H₂₈N₆ = 640.73) |
| 3-31-3 | m/z = 639.24 (C₄₅H₂₉N₅ = 639.75) | 3-32-3 | m/z = 639.24 (C₄₅H₂₉N₅ = 639.75) |
| 3-33-3 | m/z = 639.24 (C₄₅H₂₉N₅ = 639.75) | 3-34 3 | m/z = 638.25 (C₄₆H₃₀N₄ = 638.76) |
| 3-35-3 | m/z = 638.25 (C₄₆H₃₀N₄ = 638.76) | 3-36-3 | m/z = 639.24 (C₄₅H₂₉N₅ = 639.75) |
| 3-37-3 | m/z = 640.24 (C₄₄H₂₈N₆ = 640.73) | 3-38-3 | m/z = 716.27 (C₅₀H₃₂N₆ = 716.83) |
| 3-39-3 | m/z = 715.27 (C₅₁H₃₃N₅ = 715.84) | 3-40-3 | m/z = 715.27 (C₅₁H₃₃N₅ = 715.84) |
| 3-41-3 | m/z = 714.28 (C₅₂H₃₄N₄ = 714.85) | 3-42-3 | m/z = 714.28 (C₅₂H₃₄N₄ = 714.85) |
| 3-43-3 | m/z = 715.27 (C₅₁H₃₃N₅ = 715.84) | 3-44-3 | m/z = 716.27 (C₅₀H₃₂N₆ = 716.83) |
| 3-45-3 | m/z = 716.27 (C₅₀H₃₂N₆ = 716.83) | 3-46-3 | m/z = 715.27 (C₅₁H₃₃N₅ = 715.84) |
| 3-47-3 | m/z = 715.27 (C₅₁H₃₃N₅ = 715.84) | 3-48-3 | m/z = 714.28 (C₅₂H₃₄N₄ = 714.85) |
| 3-49-3 | m/z = 714.28 (C₅₂H₃₄N₄ = 714.85) | 3-50-3 | m/z = 715.27 (C₅₁H₃₃N₅ = 715.84) |
| 3-51-3 | m/z = 716.27 (C₅₀H₃₂N₆ = 716.83) | 3-52-3 | m/z = 613.23 (C₄₃H₂₇N₅ = 613.71) |
| 3-53-3 | m/z = 485.19 (C₃₅H₂₃N₃ = 485.58) | 3-54-3 | m/z = 535.20 (C₃₉H₂₅N₃ = 535.64) |
| 3-55-3 | m/z = 561.22 (C₄₁H₂₂N₃ = 561.67) | 3-56-3 | m/z = 640.24 (C₄₄H₂₈N₆ = 640.73) |
| 3-57-3 | m/z = 485.19 (C₃₅H₂₃N₃ = 485.58) | 3-58-3 | m/z = 535.20 (C₃₉H₂₅N₃ = 535.64) |
| 3-59-3 | m/z = 561.22 (C₄₁H₂₂N₃ = 561.67) | 3-60-3 | m/z = 640.24 (C₄₄H₂₈N₆ = 640.73) |
| 3-61-3 | m/z = 485.19 (C₃₅H₂₃N₃ = 485.58) | 3-62-3 | m/z = 561.22 (C₄₁H₂₂N₃ = 561.67) |
| 3-63-3 | m/z = 561.22 (C₄₁H₂₂N₃ = 561.67) | 3-64-3 | m/z = 637.25 (C₄₇H₃₁N₃ = 637.77) |
| 3-65-3 | m/z = 637.25 (C₄₇H₃₁N₃ = 637.77) | 3-66-3 | m/z = 637.25 (C₄₇H₃₁N₃ = 637.77) |
| 3-67-3 | m/z = 637.25 (C₄₇H₃₁N₃ = 637.77) | 3-68-3 | m/z = 640.24 (C₄₄H₂₈N₆ = 640.73) |
| 3-69-3 | m/z = 639.24 (C₄₅H₂₉N₅ = 639.75) | 3-70-3 | m/z = 639.24 (C₄₅H₂₉N₅ = 639.75) |
| 3-71-3 | m/z = 639.24 (C₄₅H₂₉N₅ = 639.75) | 3-72-3 | m/z = 638.25 (C₄₆H₃₀N₄ = 638.76) |
| 3-73-3 | m/z = 638.25 (C₄₆H₃₀N₄ = 638.76) | 3-74-3 | m/z = 639.24 (C₄₅H₂₉N₅ = 639.75) |
| 3-75-3 | m/z = 640.24 (C₄₄H₂₈N₆ = 640.73) | 3-76-3 | m/z = 716.27 (C₅₀H₃₂N₆ = 716.83) |
| 3-77-3 | m/z = 715.27 (C₅₁H₃₃N₅ = 715.84) | 3-78-3 | m/z = 715.27 (C₅₁H₃₃N₅ = 715.84) |
| 3-79-3 | m/z = 714.28 (C₅₂H₃₄N₄ = 714.85) | 3-80-3 | m/z = 714.28 (C₅₂H₃₄N₄ = 714.85) |
| 3-81-3 | m/z = 715.27 (C₅₁H₃₃N₅ = 715.84) | 3-82-3 | m/z = 716.27 (C₅₀H₃₂N₆ = 716.83) |
| 3-83-3 | m/z = 716.27 (C₅₀H₃₂N₆ = 716.83) | 3-84-3 | m/z = 715.27 (C₅₁H₃₃N₅ = 715.84) |
| 3-85-3 | m/z = 715.27 (C₅₁H₃₃N₅ = 715.84) | 3-86-3 | m/z = 714.28 (C₅₂H₃₄N₄ = 714.85) |
| 3-87-3 | m/z = 714.28 (C₅₂H₃₄N₄ = 714.85) | 3-88-3 | m/z = 715.27 (C₅₁H₃₃N₅ = 715.84) |
| 3-89-3 | m/z = 716.27 (C₅₀H₃₂N₆ = 716.83) | 3-90-3 | m/z = 613.23 (C₄₃H₂₇N₅ = 613.71) |
| 3-91-3 | m/z = 640.24 (C₄₄H₂₈N₆ = 640.73) | 3-92-3 | m/z = 639.24 (C₄₅H₂₉N₅ = 639.75) |
| 3-93-3 | m/z = 639.24 (C₄₅H₂₉N₅ = 639.75) | 3-94-3 | m/z = 639.24 (C₄₅H₂₉N₅ = 639.75) |
| 3-95-3 | m/z = 638.25 (C₄₆H₃₀N₄ = 638.76) | 3-96-3 | m/z = 638.25 (C₄₆H₃₀N₄ = 638.76) |
| 3-97-3 | m/z = 639.24 (C₄₅H₂₉N₅ = 639.75) | 3-98-3 | m/z = 640.24 (C₄₄H₂₈N₆ = 640.73) |
| 3-99-3 | m/z = 716.27 (C₅₀H₃₂N₆ = 716.83) | 3-100-3 | m/z = 715.27 (C₅₁H₃₃N₅ = 715.84) |
| 3-101-3 | m/z = 715.27 (C₅₁H₃₃N₅ = 715.84) | 3-102-3 | m/z = 714.28 (C₅₂H₃₄N₄ = 714.85) |
| 3-103-3 | m/z = 714.28 (C₅₂H₃₄N₄ = 714.85) | 3-104-3 | m/z = 715.27 (C₅₁H₃₃N₅ = 715.84) |
| 3-105-3 | m/z = 716.27 (C₅₀H₃₂N₆ = 716.83) | 3-106-3 | m/z = 716.27 (C₅₀H₃₂N₆ = 716.83) |
| 3-107-3 | m/z = 715.27 (C₅₁H₃₃N₅ = 715.84) | 3-108-3 | m/z = 715.27 (C₅₁H₃₃N₅ = 715.84) |
| 3-109-3 | m/z = 714.28 (C₅₂H₃₄N₄ = 714.85) | 3-110-3 | m/z = 714.28 (C₅₂H₃₄N₄ = 714.85) |
| 3-111-3 | m/z = 715.27 (C₅₁H₃₃N₅ = 715.84) | 3-112-3 | m/z = 716.27 (C₅₀H₃₂N₆ = 716.83) |
| 3-113-3 | m/z = 613.23 (C₄₃H₂₇N₅ = 613.71) | 3-114-3 | m/z = 485.19 (C₃₅H₂₃N₃ = 485.58) |
| 3-115-3 | m/z = 535.20 (C₃₉H₂₅N₃ = 535.64) | 3-116-3 | m/z = 561.22 (C₄₁H₂₂N₃ = 561.67) |
| 3-117-3 | m/z = 640.24 (C₄₄H₂₈N₆ = 640.73) | 3-118-3 | m/z = 485.19 (C₃₅H₂₃N₃ = 485.58) |
| 3-119-3 | m/z = 535.20 (C₃₉H₂₅N₃ = 535.64) | 3-120-3 | m/z = 561.22 (C₄₁H₂₂N₃ = 561.67) |
| 5-121-3 | m/z = 640.24 (C₄₄H₂₈N₆ = 640.73) | 3-122-3 | m/z = 485.19 (C₃₅H₂₃N₃ = 485.58) |
| 3-123-3 | m/z = 535.20 (C₃₉H₂₅N₃ = 535.64) | 3-124-3 | m/z = 561.22 (C₄₁H₂₂N₃ = 561.67) |
| 3-125-3 | m/z = 640.24 (C₄₄H₂₈N₆ = 640.73) | 3-126-3 | m/z = 640.24 (C₄₄H₂₈N₆ = 640.73) |
| 3-127-3 | m/z = 535.20 (C₃₉H₂₅N₃ = 535.64) | 3-128-3 | m/z = 535.20 (C₃₉H₂₅N₃ = 535.64) |
| 4-1-3 | m/z = 485.19 (C₃₅H₂₃N₃ = 485.58) | 4-2-3 | m/z = 535.20 (C₃₉H₂₅N₃ = 535.64) |
| 4-3-3 | m/z = 561.22 (C₄₁H₂₂N₃ = 561.67) | 4-4-3 | m/z = 640.24 (C₄₄H₂₈N₆ = 640.73) |
| 4-5-3 | m/z = 485.19 (C₃₅H₂₃N₃ = 485.58) | 4-6-3 | m/z = 535.20 (C₃₉H₂₅N₃ = 535.64) |
| 4-7-3 | m/z = 561.22 (C₄₁H₂₂N₃ = 561.67) | 4-8-3 | m/z = 640.24 (C₄₄H₂₈N₆ = 640.73) |
| 4-9-3 | m/z = 485.19 (C₃₅H₂₃N₃ = 485.58) | 4-10-3 | m/z = 535.20 (C₃₉H₂₅N₃ = 535.64) |
| 4-11-3 | m/z = 561.22 (C₄₁H₂₂N₃ = 561.67) | 4-12-3 | m/z = 640.24 (C₄₄H₂₈N₆ = 640.73) |
| 4-13-3 | m/z = 485.19 (C₃₅H₂₃N₃ = 485.58) | 4-14-3 | m/z = 535.20 (C₃₉H₂₅N₃ = 535.64) |
| 4-15-3 | m/z = 561.22 (C₄₁H₂₂N₃ = 561.67) | 4-16-3 | m/z = 640.24 (C₄₄H₂₈N₆ = 640.73) |
| 4-17-3 | m/z = 485.19 (C₃₅H₂₃N₃ = 485.58) | 4-18-3 | m/z = 535.20 (C₃₉H₂₅N₃ = 535.64) |
| 4-19-3 | m/z = 561.22 (C₄₁H₂₂N₃ = 561.67) | 4-20-3 | m/z = 640.24 (C₄₄H₂₈N₆ = 640.73) |
| 4-21-3 | m/z = 485.19 (C₃₅H₂₃N₃ = 485.58) | 4-22-3 | m/z = 535.20 (C₃₉H₂₅N₃ = 535.64) |
| 4-23-3 | m/z = 561.22 (C₄₁H₂₂N₃ = 561.67) | 4-24-3 | m/z = 640.24 (C₄₄H₂₈N₆ = 640.73) |
| 4-25-3 | m/z = 485.19 (C₃₅H₂₃N₃ = 485.58) | 4-26-3 | m/z = 535.20 (C₃₉H₂₅N₃ = 535.64) |
| 4-27-3 | m/z = 561.22 (C₄₁H₂₂N₃ = 561.67) | 4-28-3 | m/z = 640.24 (C₄₄H₂₈N₆ = 640.73) |
| 5-1-3 | m/z = 653.26 (C₄₆H₃₁N₅ = 653.77) | 5-3-3 | m/z = 652.26 (C₄₇H₃₂N₄ = 652.78) |
| 5-2-3 | m/z = 728.29 (C₅₃H₃₆N₄ = 728.88) | 5-4-3 | m/z = 728.29 (C₅₃H₃₆N₄ = 728.88) |

### Manufacture and Evaluation of Organic Electronic Element

### I. Manufacture and Test of Green Organic Light Emitting Element (Phosphorescent Host)

### Example 3-1 Green Organic Light Emitting Element (Phosphorescent Host)

An organic electronic light emitting element was manufactured by an ordinary method using the compound obtained through synthesis as a host material for a light emitting layer. First, a film of N¹-(naphthalen-2-yl)_N⁴,N⁴-bis(4-(naphthalen-2-yl(phenyl)amino)phenyl)-N¹-phenylbenzene-1,4-diamine (hereinafter, abbreviated as “2-TNATA”) as a hole injection layer was vacuum-deposited with a thickness of 60 nm on an ITO layer (anode) formed on a galas substrate. Then, 4,4-bis[N-(1-naphthyl)-N-phenylamino]biphenyl (hereinafter, abbreviated as “-NPD”) as a hole transport compound was vacuum-deposited on the hole injection layer to form a hole transport layer with a thickness of 60 nm. Subsequently, a light emitting layer with a thickness of 30 nm was formed on the hole transport layer by doping an upper portion of the hole transport layer with the compound 1-1-3 of the present invention as a host and Ir(ppy)₃ [tris(2-phenylpyridine)-iridium] as a dopant at a weight ratio of 95:5. Then, (1.1′-bisphenyl)-4-olato)bis(2-methyl-8-quinolinolato)aluminum (hereinafter, abbreviated as “BAlq”) was vacuum-deposited with a thickness of 10 nm for a hole blocking layer, and tris(8-quinolinol)aluminum (hereinafter, abbreviated as “Alq3”) was formed with a thickness of 40 nm for an electron injection layer. Thereafter, LiF as halogenated alkali metal was deposited with a thickness of 0.2 nm, and subsequently Al was deposited with a thickness of 150 nm, thereby using this Al/LiF as a cathode. In this way, an organic electronic light emitting element was manufactured.

### Example 3-2 to Example 3-312 Green Organic Light Emitting Element (Phosphorescent Host)

An organic electronic light emitting element was manufactured by the same method as in Example 3-1 except that, instead of compound 1-1-3 of the present invention, one of compounds 1-2-3 to 1-28-3, 2-1-3 to 2-128-3, 3-1-3 to 3-128-3, and 4-1-3 to 4-28-3 of the present invention listed on table 5 below was used as a phosphorescent host material for a light emitting layer.

### Comparative Example 3-1

An organic electronic light emitting element was manufactured by the same method as in Example 3-1 except that, instead of compound 1-1-3 of the present invention, comparative compound A [4,4′-N,N′-dicarbazole-biphenyl (CBP)] described in <Example 1> was used as a phosphorescent host material for a light emitting layer.

### Comparative Example 3-2

An organic electronic light emitting element was manufactured by the same method as in Example 3-1 except that, instead of compound 1-1-3 of the present invention, comparative compound B described in <Example 1> was used as a phosphorescent host material for a light emitting layer.

### Comparative Example 3-3

An organic electronic light emitting element was manufactured by the same method as in Example 3-1 except that, instead of compound 1-1-3 of the present invention, comparative compound C described in <Example 1> was used as a phosphorescent host material for a light emitting layer.

### Comparative Example 3-4

An organic electronic light emitting element was manufactured by the same method as in Example 3-1 except that, instead of compound 1-1-3 of the present invention, comparative compound D describe in <Example 1> was used as a phosphorescent host material for a light emitting layer.

A forward bias DC voltage was applied to the organic electronic light emitting elements manufactured in Examples 3-1 to 3-312 and Comparative Examples 3-1 to 3-4 to measure electro-luminescence (EL) characteristics thereof by PR-650 (Photoresearch), and the T95 lifetime was measured by lifetime measuring equipments (Mcscience) at reference brightness of 5000 cd/m². Table 3-5 below shows the manufacture of elements and evaluation results thereof.

TABLE 3-5

| | | | Current | Brightness | | Lifetime | CIE |
|---|---|---|---|---|---|---|---|
| | Compound | Voltage | Density | (cd/m2) | Efficiency | T(95) | (x, y) |
| Comparative | Compound | 5.8 | 23.1 | 5000.0 | 21.6 | 65.8 | (0.31, |
| Example (3-1) | (A) | | | | | | 0.60) |
| Comparative | Compound | 5.2 | 16.9 | 5000.0 | 29.5 | 98.7 | (0.31, |
| Example (3-2) | (B) | | | | | | 0.61) |
| Comparative | Compound | 5.4 | 18.7 | 5000.0 | 26.7 | 91.1 | (0.31, |
| Example (3-3) | (C) | | | | | | 0.60) |
| Comparative | Compound | 5.5 | 17.3 | 5000.0 | 28.9 | 94.3 | (0.33, |
| Example (3-4) | (D) | | | | | | 0.61) |
| Example (3-1) | Compound | 4.7 | 16.3 | 5000.0 | 30.7 | 130.6 | (0.30, |
| | (1-1-3) | | | | | | 0.60) |
| Example (3-2) | Compound | 4.6 | 14.7 | 5000.0 | 33.9 | 99.1 | (0.31, |
| | (1-2-3) | | | | | | 0.61) |
| Example (3-3) | Compound | 4.6 | 14.4 | 5000.0 | 34.6 | 145.3 | (0.31, |
| | (1-3-3) | | | | | | 0.60) |
| Example (3-4) | Compound | 4.7 | 15.3 | 5000.0 | 32.6 | 106.6 | (0.33, |
| | (1-4-3) | | | | | | 0.61) |
| Example (3-5) | Compound | 4.5 | 16.0 | 5000.0 | 31.2 | 93.4 | (0.32, |
| | (1-5-3) | | | | | | 0.61) |
| Example (3-6) | Compound | 4.9 | 15.5 | 5000.0 | 32.4 | 146.1 | (0.33, |
| | (1-6-3) | | | | | | 0.60) |
| Example (3-7) | Compound | 5.0 | 14.7 | 5000.0 | 34.1 | 94.9 | (0.32, |
| | (1-7-3) | | | | | | 0.61) |
| Example (3-8) | Compound | 4.9 | 15.1 | 5000.0 | 33.1 | 131.8 | (0.31, |
| | (1-8-3) | | | | | | 0.60) |
| Example (3-9) | Compound | 4.8 | 15.9 | 5000.0 | 31.4 | 109.5 | (0.31, |
| | (1-9-3) | | | | | | 0.61) |
| Example (3-10) | Compound | 4.7 | 14.4 | 5000.0 | 34.6 | 142.4 | (0.31, |
| | (1-10-3) | | | | | | 0.60) |
| Example (3-11) | Compound | 4.7 | 14.6 | 5000.0 | 34.3 | 129.5 | (0.33, |
| | (1-11-3) | | | | | | 0.61) |
| Example (3-12) | Compound | 4.6 | 15.4 | 5000.0 | 32.6 | 133.5 | (0.30, |
| | (1-12-3) | | | | | | 0.60) |
| Example (3-13) | Compound | 4.7 | 14.9 | 5000.0 | 33.6 | 132.9 | (0.31, |
| | (1-13-3) | | | | | | 0.61) |
| Example (3-14) | Compound | 5.0 | 16.5 | 5000.0 | 30.2 | 117.0 | (0.31, |
| | (1-14-3) | | | | | | 0.60) |
| Example (3-15) | Compound | 4.6 | 16.5 | 5000.0 | 30.3 | 107.0 | (0.33, |
| | (1-15-3) | | | | | | 0.61) |
| Example (3-16) | Compound | 4.6 | 15.5 | 5000.0 | 32.2 | 139.1 | (0.32, |
| | (1-16-3) | | | | | | 0.61) |
| Example (3-17) | Compound | 4.9 | 15.4 | 5000.0 | 32.4 | 101.4 | (0.33, |
| | (1-17-3) | | | | | | 0.60) |
| Example (3-18) | Compound | 4.6 | 15.4 | 5000.0 | 32.5 | 115.8 | (0.32, |
| | (1-18-3) | | | | | | 0.61) |
| Example (3-19) | Compound | 4.8 | 15.2 | 5000.0 | 32.9 | 148.3 | (0.31, |
| | (1-19-3) | | | | | | 0.60) |
| Example (3-20) | Compound | 4.6 | 14.7 | 5000.0 | 33.9 | 93.3 | (0.31, |
| | (1-20-3) | | | | | | 0.61) |
| Example (3-21) | Compound | 4.6 | 14.7 | 5000.0 | 33.9 | 108.7 | (0.31, |
| | (1-21-3) | | | | | | 0.60) |
| Example (3-22) | Compound | 4.5 | 15.7 | 5000.0 | 31.8 | 122.1 | (0.33, |
| | (1-22-3) | | | | | | 0.61) |
| Example (3-23) | Compound | 4.6 | 14.9 | 5000.0 | 33.5 | 145.3 | (0.30, |
| | (1-23-3) | | | | | | 0.60) |
| Example (3-24) | Compound | 5.0 | 15.0 | 5000.0 | 33.3 | 102.1 | (0.31, |
| | (1-24-3) | | | | | | 0.61) |
| Example (3-25) | Compound | 4.8 | 15.7 | 5000.0 | 31.9 | 145.8 | (0.31, |
| | (1-25-3) | | | | | | 0.60) |
| Example (3-26) | Compound | 4.7 | 15.9 | 5000.0 | 31.5 | 134.9 | (0.33, |
| | (1-26-3) | | | | | | 0.61) |
| Example (3-27) | Compound | 4.7 | 15.8 | 5000.0 | 31.6 | 95.1 | (0.32, |
| | (1-27-3) | | | | | | 0.61) |
| Example (3-28) | Compound | 4.6 | 15.1 | 5000.0 | 33.1 | 109.2 | (0.33, |
| | (1-28-3) | | | | | | 0.60) |
| Example (3-29) | Compound | 4.6 | 14.4 | 5000.0 | 34.6 | 127.9 | (0.31, |
| | (2-1-3) | | | | | | 0.61) |
| Example (3-30) | Compound | 4.8 | 15.0 | 5000.0 | 33.3 | 135.0 | (0.31, |
| | (2-2-3) | | | | | | 0.60) |
| Example (3-31) | Compound | 4.8 | 15.8 | 5000.0 | 31.6 | 123.7 | (0.33, |
| | (2-3-3) | | | | | | 0.61) |
| Example (3-32) | Compound | 4.6 | 15.3 | 5000.0 | 32.7 | 107.1 | (0.32, |
| | (2-4-3) | | | | | | 0.61) |
| Example (3-33) | Compound | 4.7 | 15.0 | 5000.0 | 33.3 | 97.1 | (0.33, |
| | (2-5-3) | | | | | | 0.60) |
| Example (3-34) | Compound | 4.9 | 15.2 | 5000.0 | 32.8 | 117.6 | (0.32, |
| | (2-6-3) | | | | | | 0.61) |
| Example (3-35) | Compound | 4.5 | 14.6 | 5000.0 | 34.3 | 140.0 | (0.31, |
| | (2-7-3) | | | | | | 0.60) |
| Example (3-36) | Compound | 4.7 | 15.9 | 5000.0 | 31.5 | 126.3 | (0.31, |
| | (2-8-3) | | | | | | 0.61) |
| Example (3-37) | Compound | 4.6 | 14.3 | 5000.0 | 35.0 | 149.7 | (0.31, |
| | (2-9-3) | | | | | | 0.60) |
| Example (3-38) | Compound | 5.0 | 15.2 | 5000.0 | 32.9 | 128.7 | (0.33, |
| | (2-10-3) | | | | | | 0.61) |
| Example (3-39) | Compound | 4.5 | 16.6 | 5000.0 | 30.2 | 125.3 | (0.30, |
| | (2-11-3) | | | | | | 0.60) |
| Example (3-40) | Compound | 4.5 | 15.5 | 5000.0 | 32.4 | 116.3 | (0.31, |
| | (2-12-3) | | | | | | 0.61) |
| Example (3-41) | Compound | 4.8 | 16.0 | 5000.0 | 31.3 | 113.8 | (0.31, |
| | (2-13-3) | | | | | | 0.60) |
| Example (3-42) | Compound | 4.6 | 16.6 | 5000.0 | 30.1 | 99.7 | (0.33, |
| | (2-14-3) | | | | | | 0.61) |
| Example (3-43) | Compound | 4.7 | 15.4 | 5000.0 | 32.5 | 90.9 | (0.32, |
| | (2-15-3) | | | | | | 0.61) |
| Example (3-44) | Compound | 4.8 | 14.7 | 5000.0 | 34.0 | 101.2 | (0.33, |
| | (2-16-3) | | | | | | 0.60) |
| Example (3-45) | Compound | 4.9 | 15.2 | 5000.0 | 33.0 | 137.2 | (0.32, |
| | (2-17-3) | | | | | | 0.61) |
| Example (3-46) | Compound | 4.9 | 16.3 | 5000.0 | 30.7 | 100.7 | (0.31, |
| | (2-18-3) | | | | | | 0.60) |
| Example (3-47) | Compound | 4.7 | 15.3 | 5000.0 | 32.6 | 94.6 | (0.31, |
| | (2-19-3) | | | | | | 0.61) |
| Example (3-48) | Compound | 4.7 | 14.4 | 5000.0 | 34.7 | 105.7 | (0.31, |
| | (2-20-3) | | | | | | 0.60) |
| Example (3-49) | Compound | 4.8 | 16.0 | 5000.0 | 31.2 | 105.4 | (0.33, |
| | (2-21-3) | | | | | | 0.61) |
| Example (3-50) | Compound | 4.5 | 15.9 | 5000.0 | 31.5 | 122.8 | (0.30, |
| | (2-22-3) | | | | | | 0.60) |
| Example (3-51) | Compound | 4.5 | 15.7 | 5000.0 | 31.9 | 96.6 | (0.31, |
| | (2-23-3) | | | | | | 0.61) |
| Example (3-52) | Compound | 4.5 | 14.3 | 5000.0 | 34.9 | 136.1 | (0.31, |
| | (2-24-3) | | | | | | 0.60) |
| Example (3-53) | Compound | 5.0 | 15.7 | 5000.0 | 31.9 | 140.8 | (0.33, |
| | (2-25-3) | | | | | | 0.61) |
| Example (3-54) | Compound | 4.6 | 15.7 | 5000.0 | 31.9 | 104.2 | (0.32, |
| | (2-26-3) | | | | | | 0.61) |
| Example (3-55) | Compound | 4.9 | 14.9 | 5000.0 | 33.6 | 124.8 | (0.33, |
| | (2-27-3) | | | | | | 0.60) |
| Example (3-56) | Compound | 4.9 | 14.3 | 5000.0 | 34.8 | 108.6 | (0.32, |
| | (2-28-3) | | | | | | 0.61) |
| Example (3-57) | Compound | 4.6 | 14.7 | 5000.0 | 34.0 | 125.7 | (0.31, |
| | (2-29-3) | | | | | | 0.60) |
| Example (3-58) | Compound | 4.7 | 15.8 | 5000.0 | 31.6 | 100.5 | (0.33, |
| | (2-30-3) | | | | | | 0.61) |
| Example (3-59) | Compound | 4.9 | 15.3 | 5000.0 | 32.8 | 142.8 | (0.30, |
| | (2-31-3) | | | | | | 0.60) |
| Example (3-60) | Compound | 4.9 | 16.1 | 5000.0 | 31.0 | 110.7 | (0.31, |
| | (2-32-3) | | | | | | 0.61) |
| Example (3-61) | Compound | 4.6 | 15.3 | 5000.0 | 32.7 | 90.4 | (0.31, |
| | (2-33-3) | | | | | | 0.60) |
| Example (3-62) | Compound | 4.6 | 14.7 | 5000.0 | 34.0 | 144.5 | (0.33, |
| | (2-34-3) | | | | | | 0.61) |
| Example (3-63) | Compound | 4.8 | 15.4 | 5000.0 | 32.4 | 132.6 | (0.32, |
| | (2-35-3) | | | | | | 0.61) |
| Example (3-64) | Compound | 4.6 | 14.7 | 5000.0 | 33.9 | 107.6 | (0.33, |
| | (2-36-3) | | | | | | 0.60) |
| Example (3-65) | Compound | 4.8 | 16.4 | 5000.0 | 30.5 | 128.1 | (0.32, |
| | (2-37-3) | | | | | | 0.61) |
| Example (3-66) | Compound | 4.7 | 14.3 | 5000.0 | 34.9 | 114.1 | (0.31, |
| | (2-38-3) | | | | | | 0.60) |
| Example (3-67) | Compound | 5.0 | 14.6 | 5000.0 | 34.1 | 101.3 | (0.31, |
| | (2-39-3) | | | | | | 0.61) |
| Example (3-68) | Compound | 4.7 | 16.0 | 5000.0 | 31.3 | 93.2 | (0.31, |
| | (2-40-3) | | | | | | 0.60) |
| Example (3-69) | Compound | 4.5 | 14.8 | 5000.0 | 33.7 | 134.8 | (0.33, |
| | (2-41-3) | | | | | | 0.61) |
| Example (3-70) | Compound | 4.9 | 15.0 | 5000.0 | 33.4 | 111.8 | (0.30, |
| | (2-42-3) | | | | | | 0.60) |
| Example (3-71) | Compound | 4.9 | 14.9 | 5000.0 | 33.7 | 132.2 | (0.31, |
| | (2-43-3) | | | | | | 0.61) |
| Example (3-72) | Compound | 4.8 | 15.0 | 5000.0 | 33.3 | 124.0 | (0.31, |
| | (2-44-3) | | | | | | 0.60) |
| Example (3-73) | Compound | 4.8 | 14.9 | 5000.0 | 33.6 | 101.3 | (0.33, |
| | (2-45-3) | | | | | | 0.61) |
| Example (3-74) | Compound | 4.5 | 15.2 | 5000.0 | 32.9 | 95.4 | (0.32, |
| | (2-46-3) | | | | | | 0.61) |
| Example (3-75) | Compound | 4.8 | 14.8 | 5000.0 | 33.8 | 115.0 | (0.33, |
| | (2-47-3) | | | | | | 0.61) |
| Example (3-76) | Compound | 4.8 | 15.8 | 5000.0 | 31.6 | 136.5 | (0.30, |
| | (2-48-3) | | | | | | 0.60) |
| Example (3-77) | Compound | 4.6 | 14.9 | 5000.0 | 33.5 | 136.8 | (0.31, |
| | (2-49-3) | | | | | | 0.61) |
| Example (3-78) | Compound | 4.8 | 15.1 | 5000.0 | 33.2 | 125.7 | (0.31, |
| | (2-50-3) | | | | | | 0.60) |
| Example (3-79) | Compound | 4.9 | 16.7 | 5000.0 | 30.0 | 149.8 | (0.31, |
| | (2-51-3) | | | | | | 0.61) |
| Example (3-80) | Compound | 5.0 | 15.6 | 5000.0 | 32.1 | 142.4 | (0.31, |
| | (2-52-3) | | | | | | 0.60) |
| Example (3-81) | Compound | 4.7 | 15.2 | 5000.0 | 33.0 | 128.9 | (0.33, |
| | (2-53-3) | | | | | | 0.61) |
| Example (3-82) | Compound | 4.7 | 14.4 | 5000.0 | 34.7 | 147.9 | (0.32, |
| | (2-54-3) | | | | | | 0.61) |
| Example (3-83) | Compound | 4.8 | 15.2 | 5000.0 | 32.8 | 147.9 | (0.33, |
| | (2-55-3) | | | | | | 0.60) |
| Example (3-84) | Compound | 4.5 | 15.0 | 5000.0 | 33.2 | 124.2 | (0.32, |
| | (2-56-3) | | | | | | 0.61) |
| Example (3-85) | Compound | 4.6 | 14.8 | 5000.0 | 33.8 | 122.9 | (0.31, |
| | (2-57-3) | | | | | | 0.60) |
| Example (3-86) | Compound | 4.6 | 15.7 | 5000.0 | 31.8 | 118.1 | (0.31, |
| | (2-58-3) | | | | | | 0.61) |
| Example (3-87) | Compound | 4.5 | 15.7 | 5000.0 | 31.8 | 120.6 | (0.31, |
| | (2-59-3) | | | | | | 0.60) |
| Example (3-88) | Compound | 4.6 | 16.3 | 5000.0 | 30.6 | 92.6 | (0.33, |
| | (2-60-3) | | | | | | 0.61) |
| Example (3-89) | Compound | 4.6 | 15.0 | 5000.0 | 33.4 | 117.5 | (0.30, |
| | (2-61-3) | | | | | | 0.60) |
| Example (3-90) | Compound | 4.8 | 15.8 | 5000.0 | 31.7 | 111.4 | (0.31, |
| | (2-62-3) | | | | | | 0.61) |
| Example (3-91) | Compound | 5.0 | 15.5 | 5000.0 | 32.3 | 122.5 | (0.31, |
| | (2-63-3) | | | | | | 0.60) |
| Example (3-92) | Compound | 4.9 | 14.7 | 5000.0 | 34.0 | 117.5 | (0.33, |
| | (2-64-3) | | | | | | 0.61) |
| Example (3-93) | Compound | 4.9 | 15.6 | 5000.0 | 32.0 | 147.6 | (0.32, |
| | (2-65-3) | | | | | | 0.61) |
| Example (3-94) | Compound | 4.8 | 14.8 | 5000.0 | 33.9 | 123.8 | (0.33, |
| | (2-66-3) | | | | | | 0.60) |
| Example (3-95) | Compound | 4.6 | 16.0 | 5000.0 | 31.2 | 135.5 | (0.32, |
| | (2-67-3) | | | | | | 0.61) |
| Example (3-96) | Compound | 5.0 | 14.3 | 5000.0 | 34.9 | 91.2 | (0.31, |
| | (2-68-3) | | | | | | 0.60) |
| Example (3-97) | Compound | 4.6 | 16.4 | 5000.0 | 30.5 | 132.1 | (0.31, |
| | (2-69-3) | | | | | | 0.61) |
| Example (3-98) | Compound | 4.9 | 16.1 | 5000.0 | 31.1 | 123.1 | (0.31, |
| | (2-70-3) | | | | | | 0.60) |
| Example (3-99) | Compound | 4.9 | 15.4 | 5000.0 | 32.6 | 141.6 | (0.33, |
| | (2-71-3) | | | | | | 0.61) |
| Example (3-100) | Compound | 4.6 | 15.5 | 5000.0 | 32.3 | 149.6 | (0.30, |
| | (2-72-3) | | | | | | 0.60) |
| Example (3-101) | Compound | 4.9 | 14.8 | 5000.0 | 33.8 | 139.3 | (0.31, |
| | (2-73-3) | | | | | | 0.61) |
| Example (3-102) | Compound | 5.0 | 16.1 | 5000.0 | 31.1 | 133.2 | (0.31, |
| | (2-74-3) | | | | | | 0.60) |
| Example (3-103) | Compound | 4.5 | 14.9 | 5000.0 | 33.5 | 136.4 | (0.33, |
| | (2-75-3) | | | | | | 0.61) |
| Example (3-104) | Compound | 4.7 | 15.0 | 5000.0 | 33.3 | 99.5 | (0.32, |
| | (2-76-3) | | | | | | 0.61) |
| Example (3-105) | Compound | 4.6 | 16.1 | 5000.0 | 31.1 | 142.1 | (0.33, |
| | (2-77-3) | | | | | | 0.60) |
| Example (3-106) | Compound | 4.7 | 16.3 | 5000.0 | 30.6 | 129.3 | (0.32, |
| | (2-78-3) | | | | | | 0.61) |
| Example (3-107) | Compound | 4.5 | 16.0 | 5000.0 | 31.2 | 122.7 | (0.31, |
| | (2-79-3) | | | | | | 0.60) |
| Example (3-108) | Compound | 4.5 | 16.2 | 5000.0 | 30.8 | 144.6 | (0.33, |
| | (2-80-3) | | | | | | 0.61) |
| Example (3-109) | Compound | 5.0 | 16.1 | 5000.0 | 31.1 | 149.8 | (0.30, |
| | (2-81-3) | | | | | | 0.60) |
| Example (3-110) | Compound | 4.6 | 16.4 | 5000.0 | 30.5 | 93.1 | (0.31, |
| | (2-82-3) | | | | | | 0.61) |
| Example (3-111) | Compound | 4.7 | 15.2 | 5000.0 | 32.8 | 135.3 | (0.31, |
| | (2-83-3) | | | | | | 0.60) |
| Example (3-112) | Compound | 5.0 | 14.5 | 5000.0 | 34.6 | 136.8 | (0.33, |
| | (2-84-3) | | | | | | 0.61) |
| Example (3-113) | Compound | 4.5 | 14.5 | 5000.0 | 34.5 | 92.0 | (0.32, |
| | (2-85-3) | | | | | | 0.61) |
| Example (3-114) | Compound | 4.5 | 16.1 | 5000.0 | 31.0 | 142.9 | (0.33, |
| | (2-86-3) | | | | | | 0.60) |
| Example (3-115) | Compound | 4.5 | 14.5 | 5000.0 | 34.4 | 99.0 | (0.32, |
| | (2-87-3) | | | | | | 0.61) |
| Example (3-116) | Compound | 4.7 | 15.2 | 5000.0 | 32.9 | 103.5 | (0.31, |
| | (2-88-3) | | | | | | 0.60) |
| Example (3-117) | Compound | 4.6 | 14.6 | 5000.0 | 34.3 | 120.8 | (0.31, |
| | (2-89-3) | | | | | | 0.61) |
| Example (3-118) | Compound | 4.9 | 15.0 | 5000.0 | 33.2 | 140.9 | (0.31, |
| | (2-90-3) | | | | | | 0.60) |
| Example (3-119) | Compound | 4.8 | 16.0 | 5000.0 | 31.2 | 110.0 | (0.33, |
| | (2-91-3) | | | | | | 0.61) |
| Example (3-120) | Compound | 4.9 | 15.0 | 5000.0 | 33.4 | 128.2 | (0.30, |
| | (2-92-3) | | | | | | 0.60) |
| Example (3-121) | Compound | 4.9 | 16.4 | 5000.0 | 30.5 | 140.2 | (0.31, |
| | (2-93-3) | | | | | | 0.61) |
| Example (3-122) | Compound | 4.8 | 15.6 | 5000.0 | 32.1 | 141.4 | (0.31, |
| | (2-94-3) | | | | | | 0.60) |
| Example (3-123) | Compound | 4.6 | 15.1 | 5000.0 | 33.1 | 134.2 | (0.33, |
| | (2-95-3) | | | | | | 0.61) |
| Example (3-124) | Compound | 4.5 | 15.7 | 5000.0 | 31.9 | 137.6 | (0.32, |
| | (2-96-3) | | | | | | 0.61) |
| Example (3-125) | Compound | 4.7 | 16.0 | 5000.0 | 31.2 | 94.7 | (0.33, |
| | (2-97-3) | | | | | | 0.61) |
| Example (3-126) | Compound | 4.9 | 14.4 | 5000.0 | 34.7 | 140.1 | (0.30, |
| | (2-98-3) | | | | | | 0.60) |
| Example (3-127) | Compound | 5.0 | 16.4 | 5000.0 | 30.6 | 132.9 | (0.32, |
| | (2-99-3) | | | | | | 0.61) |
| Example (3-128) | Compound | 4.6 | 15.1 | 5000.0 | 33.1 | 124.4 | (0.31, |
| | (2-100-3) | | | | | | 0.60) |
| Example (3-129) | Compound | 4.9 | 14.8 | 5000.0 | 33.7 | 127.7 | (0.30, |
| | (2-101-3) | | | | | | 0.60) |
| Example (3-130) | Compound | 4.6 | 15.9 | 5000.0 | 31.5 | 111.0 | (0.31, |
| | (2-102-3) | | | | | | 0.61) |
| Example (3-131) | Compound | 5.0 | 15.5 | 5000.0 | 32.3 | 135.6 | (0.31, |
| | (2-103-3) | | | | | | 0.60) |
| Example (3-132) | Compound | 5.0 | 14.8 | 5000.0 | 33.9 | 99.1 | (0.33, |
| | (2-104-3) | | | | | | 0.61) |
| Example (3-133) | Compound | 4.9 | 14.6 | 5000.0 | 34.2 | 127.5 | (0.32, |
| | (2-105-3) | | | | | | 0.61) |
| Example (3-134) | Compound | 4.9 | 15.1 | 5000.0 | 33.1 | 92.6 | (0.33, |
| | (2-106-3) | | | | | | 0.60) |
| Example (3-135) | Compound | 4.7 | 15.2 | 5000.0 | 33.0 | 121.2 | (0.32, |
| | (2-107-3) | | | | | | 0.61) |
| Example (3-136) | Compound | 4.7 | 14.4 | 5000.0 | 34.7 | 98.3 | (0.31, |
| | (2-108-3) | | | | | | 0.60) |
| Example (3-137) | Compound | 4.6 | 15.1 | 5000.0 | 33.1 | 102.6 | (0.31, |
| | (2-109-3) | | | | | | 0.61) |
| Example (3-138) | Compound | 4.6 | 16.6 | 5000.0 | 30.1 | 115.8 | (0.31, |
| | (2-110-3) | | | | | | 0.60) |
| Example (3-139) | Compound | 4.9 | 16.4 | 5000.0 | 30.4 | 111.0 | (0.33, |
| | (2-111-3) | | | | | | 0.61) |
| Example (3-140) | Compound | 4.8 | 16.4 | 5000.0 | 30.5 | 125.1 | (0.30, |
| | (2-112-3) | | | | | | 0.60) |
| Example (3-141) | Compound | 5.0 | 16.6 | 5000.0 | 30.2 | 99.0 | (0.31, |
| | (2-113-3) | | | | | | 0.61) |
| Example (3-142) | Compound | 4.8 | 15.1 | 5000.0 | 33.1 | 107.6 | (0.31, |
| | (2-114-3) | | | | | | 0.60) |
| Example (3-143) | Compound | 4.5 | 16.4 | 5000.0 | 30.5 | 139.6 | (0.33, |
| | (2-115-3) | | | | | | 0.61) |
| Example (3-144) | Compound | 5.0 | 15.6 | 5000.0 | 32.0 | 142.6 | (0.32, |
| | (2-116-3) | | | | | | 0.61) |
| Example (3-145) | Compound | 4.6 | 16.5 | 5000.0 | 30.3 | 105.6 | (0.33, |
| | (2-117-3) | | | | | | 0.60) |
| Example (3-146) | Compound | 4.6 | 14.8 | 5000.0 | 33.8 | 95.3 | (0.32, |
| | (2-118-3) | | | | | | 0.61) |
| Example (3-147) | Compound | 4.6 | 15.2 | 5000.0 | 32.8 | 126.4 | (0.31, |
| | (2-119-3) | | | | | | 0.60) |
| Example (3-148) | Compound | 4.9 | 15.1 | 5000.0 | 33.0 | 109.3 | (0.31, |
| | (2-120-3) | | | | | | 0.61) |
| Example (3-149) | Compound | 4.9 | 16.6 | 5000.0 | 30.1 | 130.1 | (0.31, |
| | (2-121-3) | | | | | | 0.60) |
| Example (3-150) | Compound | 4.6 | 15.7 | 5000.0 | 31.9 | 124.0 | (0.33, |
| | (2-122-3) | | | | | | 0.61) |
| Example (3-151) | Compound | 4.5 | 16.4 | 5000.0 | 30.5 | 138.7 | (0.30, |
| | (2-123-3 | | | | | | 0.60) |
| Example (3-152) | Compound | 4.7 | 14.6 | 5000.0 | 34.2 | 143.6 | (0.31, |
| | (2-124-3) | | | | | | 0.61) |
| Example (3-153) | Compound | 4.9 | 16.6 | 5000.0 | 30.1 | 102.4 | (0.31, |
| | (2-125-3) | | | | | | 0.60) |
| Example (3-154) | Compound | 4.8 | 16.3 | 5000.0 | 30.8 | 109.2 | (0.33, |
| | (2-126-3) | | | | | | 0.61) |
| Example (3-155) | Compound | 4.6 | 16.2 | 5000.0 | 30.8 | 115.8 | (0.32, |
| | (2-127-3) | | | | | | 0.61) |
| Example (3-156) | Compound | 4.8 | 15.9 | 5000.0 | 31.5 | 134.1 | (0.33, |
| | (2-128-3) | | | | | | 0.60) |
| Example (3-157) | Compound | 4.6 | 13.9 | 5000.0 | 35.9 | 105.0 | (0.31, |
| | (3-1-3) | | | | | | 0.61) |
| Example (3-158) | Compound | 5.0 | 13.9 | 5000.0 | 36.0 | 107.5 | (0.31, |
| | (3-2-3) | | | | | | 0.60) |
| Example (3-159) | Compound | 4.7 | 14.2 | 5000.0 | 35.2 | 146.5 | (0.33, |
| | (3-3-3) | | | | | | 0.61) |
| Example (3-160) | Compound | 4.6 | 13.8 | 5000.0 | 36.2 | 114.9 | (0.32, |
| | (3-4-3) | | | | | | 0.61) |
| Example (3-161) | Compound | 4.9 | 14.1 | 5000.0 | 35.5 | 138.7 | (0.33, |
| | (3-5-3) | | | | | | 0.60) |
| Example (3-162) | Compound | 4.9 | 13.6 | 5000.0 | 36.9 | 96.4 | (0.32, |
| | (3-6-3) | | | | | | 0.61) |
| Example (3-163) | Compound | 4.8 | 13.5 | 5000.0 | 36.9 | 97.7 | (0.31, |
| | (3-7-3) | | | | | | 0.60) |
| Example (3-164) | Compound | 4.7 | 13.5 | 5000.0 | 36.9 | 142.4 | (0.31, |
| | (3-8-3) | | | | | | 0.61) |
| Example (3-165) | Compound | 4.9 | 14.0 | 5000.0 | 35.8 | 111.5 | (0.31, |
| | (3-9-3) | | | | | | 0.60) |
| Example (3-166) | Compound | 4.6 | 14.3 | 5000.0 | 35.0 | 91.2 | (0.33, |
| | (3-10-3) | | | | | | 0.61) |
| Example (3-167) | Compound | 4.9 | 14.2 | 5000.0 | 35.1 | 137.8 | (0.30, |
| | (3-11-3) | | | | | | 0.60) |
| Example (3-168) | Compound | 4.6 | 13.7 | 5000.0 | 36.5 | 123.2 | (0.31, |
| | (3-12-3) | | | | | | 0.61) |
| Example (3-169) | Compound | 4.6 | 13.9 | 5000.0 | 35.9 | 105.0 | (0.31, |
| | (3-13-3) | | | | | | 0.60) |
| Example (3-170) | Compound | 4.6 | 13.7 | 5000.0 | 36.6 | 116.8 | (0.33, |
| | (3-14-3) | | | | | | 0.61) |
| Example (3-171) | Compound | 4.7 | 13.6 | 5000.0 | 36.7 | 99.6 | (0.32, |
| | (3-15-3) | | | | | | 0.61) |
| Example (3-172) | Compound | 4.9 | 14.1 | 5000.0 | 35.4 | 101.8 | (0.33, |
| | (3-16-3) | | | | | | 0.60) |
| Example (3-173) | Compound | 4.7 | 13.9 | 5000.0 | 35.9 | 93.0 | (0.32, |
| | (3-17-3) | | | | | | 0.61) |
| Example (3-174) | Compound | 4.9 | 13.6 | 5000.0 | 36.8 | 112.3 | (0.31, |
| | (3-18-3) | | | | | | 0.60) |
| Example (3-175) | Compound | 4.7 | 14.1 | 5000.0 | 35.6 | 143.9 | (0.31, |
| | (3-19-3) | | | | | | 0.61) |
| Example (3-176) | Compound | 4.8 | 14.0 | 5000.0 | 35.6 | 147.3 | (0.31, |
| | (3-20-3) | | | | | | 0.60) |
| Example (3-177) | Compound | 4.6 | 14.0 | 5000.0 | 35.6 | 118.5 | (0.33, |
| | (3-21-3) | | | | | | 0.61) |
| Example (3-178) | Compound | 4.5 | 14.3 | 5000.0 | 35.1 | 130.8 | (0.30, |
| | (3-22-3) | | | | | | 0.60) |
| Example (3-179) | Compound | 4.9 | 13.9 | 5000.0 | 35.9 | 135.3 | (0.31, |
| | (3-23-3) | | | | | | 0.61) |
| Example (3-180) | Compound | 4.6 | 14.0 | 5000.0 | 35.8 | 125.6 | (0.31, |
| | (3-24-3) | | | | | | 0.60) |
| Example (3-181) | Compound | 4.8 | 13.6 | 5000.0 | 36.7 | 142.7 | (0.33, |
| | (3-25-3) | | | | | | 0.61) |
| Example (3-182) | Compound | 5.0 | 13.8 | 5000.0 | 36.3 | 132.2 | (0.32, |
| | (3-26-3) | | | | | | 0.61) |
| Example (3-183) | Compound | 4.7 | 13.9 | 5000.0 | 36.0 | 107.1 | (0.33, |
| | (3-27-3) | | | | | | 0.60) |
| Example (3-184) | Compound | 4.6 | 13.9 | 5000.0 | 35.8 | 106.3 | (0.32, |
| | (3-28-3) | | | | | | 0.61) |
| Example (3-185) | Compound | 4.9 | 14.1 | 5000.0 | 35.4 | 94.9 | (0.31, |
| | (3-29-3) | | | | | | 0.60) |
| Example (3-186) | Compound | 4.9 | 13.9 | 5000.0 | 36.0 | 125.3 | (0.33, |
| | (3-30-3) | | | | | | 0.61) |
| Example (3-187) | Compound | 5.0 | 13.7 | 5000.0 | 36.6 | 97.0 | (0.30, |
| | (3-31-3) | | | | | | 0.60) |
| Example (3-188) | Compound | 4.8 | 14.1 | 5000.0 | 35.4 | 136.9 | (0.31, |
| | (3-32-3) | | | | | | 0.61) |
| Example (3-189) | Compound | 4.9 | 14.1 | 5000.0 | 35.6 | 138.2 | (0.31, |
| | (3-33-3) | | | | | | 0.60) |
| Example (3-190) | Compound | 4.8 | 14.1 | 5000.0 | 35.4 | 144.0 | (0.33, |
| | (3-34-3) | | | | | | 0.61) |
| Example (3-191) | Compound | 4.6 | 13.8 | 5000.0 | 36.3 | 131.4 | (0.32, |
| | (3-35-3) | | | | | | 0.61) |
| Example (3-192) | Compound | 4.9 | 13.9 | 5000.0 | 36.1 | 114.2 | (0.33, |
| | (3-36-3) | | | | | | 0.60) |
| Example (3-193) | Compound | 4.7 | 14.2 | 5000.0 | 35.3 | 139.2 | (0.32, |
| | (3-37-3) | | | | | | 0.61) |
| Example (3-194) | Compound | 4.8 | 14.0 | 5000.0 | 35.6 | 96.0 | (0.31, |
| | (3-38-3) | | | | | | 0.60) |
| Example (3-195) | Compound | 5.0 | 13.6 | 5000.0 | 36.7 | 99.6 | (0.31, |
| | (3-39-3) | | | | | | 0.61) |
| Example (3-196) | Compound | 5.0 | 14.0 | 5000.0 | 35.6 | 119.2 | (0.31, |
| | (3-40-3) | | | | | | 0.60) |
| Example (3-197) | Compound | 5.0 | 13.8 | 5000.0 | 36.2 | 120.9 | (0.33, |
| | (3-41-3) | | | | | | 0.61) |
| Example (3-198) | Compound | 4.6 | 14.3 | 5000.0 | 35.1 | 123.3 | (0.30, |
| | (3-42-3) | | | | | | 0.60) |
| Example (3-199) | Compound | 4.7 | 13.9 | 5000.0 | 36.1 | 132.8 | (0.31, |
| | (3-43-3) | | | | | | 0.61) |
| Example (3-200) | Compound | 4.6 | 13.6 | 5000.0 | 36.7 | 135.2 | (0.31, |
| | (3-44-3) | | | | | | 0.60) |
| Example (3-201) | Compound | 4.9 | 13.6 | 5000.0 | 36.8 | 142.5 | (0.33, |
| | (3-45-3) | | | | | | 0.61) |
| Example (3-202) | Compound | 4.9 | 13.8 | 5000.0 | 36.3 | 121.2 | (0.32, |
| | (3-46-3) | | | | | | 0.61) |
| Example (3-203) | Compound | 4.6 | 14.3 | 5000.0 | 35.0 | 95.2 | (0.33, |
| | (3-47-3) | | | | | | 0.61) |
| Example (3-204) | Compound | 4.9 | 14.2 | 5000.0 | 35.2 | 146.2 | (0.30, |
| | (3-48-3) | | | | | | 0.60) |
| Example (3-205) | Compound | 4.6 | 14.1 | 5000.0 | 35.5 | 105.3 | (0.32, |
| | (3-49-3) | | | | | | 0.61) |
| Example (3-206) | Compound | 4.9 | 13.6 | 5000.0 | 36.7 | 140.0 | (0.31, |
| | (3-50-3) | | | | | | 0.60) |
| Example (3-207) | Compound | 4.6 | 13.6 | 5000.0 | 36.7 | 119.0 | (0.31, |
| | (3-51-3) | | | | | | 0.61) |
| Example (3-208) | Compound | 4.9 | 14.1 | 5000.0 | 35.6 | 112.3 | (0.31, |
| | (3-52-3) | | | | | | 0.60) |
| Example (3-209) | Compound | 4.7 | 14.2 | 5000.0 | 35.2 | 123.2 | (0.33, |
| | (3-53-3) | | | | | | 0.61) |
| Example (3-210) | Compound | 4.9 | 13.9 | 5000.0 | 36.0 | 106.5 | (0.32, |
| | (3-54-3) | | | | | | 0.61) |
| Example (3-211) | Compound | 4.6 | 14.2 | 5000.0 | 35.2 | 109.0 | (0.33, |
| | (3-55-3) | | | | | | 0.60) |
| Example (3-212) | Compound | 4.9 | 13.6 | 5000.0 | 36.8 | 145.1 | (0.32, |
| | (3-56-3) | | | | | | 0.61) |
| Example (3-213) | Compound | 4.6 | 13.6 | 5000.0 | 36.8 | 106.6 | (0.31, |
| | (3-57-3) | | | | | | 0.60) |
| Example (3-214) | Compound | 4.7 | 14.0 | 5000.0 | 35.6 | 124.2 | (0.31, |
| | (3-58-3) | | | | | | 0.61) |
| Example (3-215) | Compound | 4.9 | 14.1 | 5000.0 | 35.5 | 119.4 | (0.31, |
| | (3-59-3) | | | | | | 0.60) |
| Example (3-216) | Compound | 4.6 | 13.6 | 5000.0 | 36.7 | 105.5 | (0.33, |
| | (3-60-3) | | | | | | 0.61) |
| Example (3-217) | Compound | 4.8 | 13.9 | 5000.0 | 36.0 | 108.5 | (0.30, |
| | (3-61-3) | | | | | | 0.60) |
| Example (3-218) | Compound | 4.9 | 14.1 | 5000.0 | 35.4 | 116.0 | (0.31, |
| | (3-62-3) | | | | | | 0.61) |
| Example (3-219) | Compound | 4.8 | 13.8 | 5000.0 | 36.2 | 100.0 | (0.31, |
| | (3-63-3) | | | | | | 0.60) |
| Example (3-220) | Compound | 4.5 | 14.0 | 5000.0 | 35.8 | 146.0 | (0.33, |
| | (3-64-3) | | | | | | 0.61) |
| Example (3-221) | Compound | 5.0 | 13.7 | 5000.0 | 36.6 | 96.7 | (0.32, |
| | (3-65-3) | | | | | | 0.61) |
| Example (3-222) | Compound | 4.7 | 13.6 | 5000.0 | 36.7 | 103.7 | (0.33, |
| | (3-66-3) | | | | | | 0.60) |
| Example (3-223) | Compound | 4.8 | 13.9 | 5000.0 | 36.1 | 105.2 | (0.32, |
| | (3-67-3) | | | | | | 0.61) |
| Example (3-224) | Compound | 4.9 | 13.8 | 5000.0 | 36.1 | 110.4 | (0.31, |
| | (3-68-3) | | | | | | 0.60) |
| Example (3-225) | Compound | 4.8 | 13.5 | 5000.0 | 36.9 | 132.3 | (0.31, |
| | (3-69-3) | | | | | | 0.61) |
| Example (3-226) | Compound | 4.6 | 13.6 | 5000.0 | 36.8 | 111.4 | (0.31, |
| | (3-70-3) | | | | | | 0.60) |
| Example (3-227) | Compound | 4.7 | 14.0 | 5000.0 | 35.6 | 105.6 | (0.33, |
| | (3-71-3) | | | | | | 0.61) |
| Example (3-228) | Compound | 4.5 | 13.6 | 5000.0 | 36.8 | 135.0 | (0.30, |
| | (3-72-3) | | | | | | 0.60) |
| Example (3-229) | Compound | 4.7 | 14.2 | 5000.0 | 35.2 | 134.9 | (0.31, |
| | (3-73-3) | | | | | | 0.61) |
| Example (3-230) | Compound | 4.9 | 14.3 | 5000.0 | 35.0 | 133.3 | (0.31, |
| | (3-74-3) | | | | | | 0.60) |
| Example (3-231) | Compound | 4.6 | 13.7 | 5000.0 | 36.4 | 117.0 | (0.33, |
| | (3-75-3) | | | | | | 0.61) |
| Example (3-232) | Compound | 4.9 | 13.7 | 5000.0 | 36.4 | 113.7 | (0.32, |
| | (3-76-3) | | | | | | 0.61) |
| Example (3-233) | Compound | 4.6 | 13.6 | 5000.0 | 36.8 | 138.2 | (0.33, |
| | (3-77-3) | | | | | | 0.60) |
| Example (3-234) | Compound | 4.8 | 13.7 | 5000.0 | 36.4 | 136.1 | (0.32, |
| | (3-78-3) | | | | | | 0.61) |
| Example (3-235) | Compound | 4.6 | 13.5 | 5000.0 | 36.9 | 139.2 | (0.31, |
| | (3-79-3) | | | | | | 0.60) |
| Example (3-236) | Compound | 4.8 | 14.3 | 5000.0 | 35.0 | 117.2 | (0.33, |
| | (3-80-3) | | | | | | 0.61) |
| Example (3-237) | Compound | 4.8 | 14.1 | 5000.0 | 35.5 | 145.3 | (0.30, |
| | (3-81-3) | | | | | | 0.60) |
| Example (3-238) | Compound | 4.7 | 14.1 | 5000.0 | 35.5 | 140.3 | (0.31, |
| | (3-82-3) | | | | | | 0.61) |
| Example (3-239) | Compound | 5.0 | 13.8 | 5000.0 | 36.2 | 104.9 | (0.31, |
| | (3-83-3) | | | | | | 0.60) |
| Example (3-240) | Compound | 4.6 | 13.8 | 5000.0 | 36.2 | 104.1 | (0.33, |
| | (3-84-3) | | | | | | 0.61) |
| Example (3-241) | Compound | 4.9 | 14.3 | 5000.0 | 35.0 | 100.3 | (0.32, |
| | (3-85-3) | | | | | | 0.61) |
| Example (3-242) | Compound | 4.8 | 14.2 | 5000.0 | 35.2 | 116.7 | (0.33, |
| | (3-86-3) | | | | | | 0.60) |
| Example (3-243) | Compound | 4.9 | 14.0 | 5000.0 | 35.8 | 135.6 | (0.32, |
| | (3-87-3) | | | | | | 0.61) |
| Example (3-244) | Compound | 4.7 | 13.6 | 5000.0 | 36.8 | 130.9 | (0.31, |
| | (3-88-3) | | | | | | 0.60) |
| Example (3-245) | Compound | 4.6 | 13.7 | 5000.0 | 36.5 | 107.7 | (0.31, |
| | (3-89-3) | | | | | | 0.61) |
| Example (3-246) | Compound | 4.5 | 14.1 | 5000.0 | 35.5 | 102.5 | (0.31, |
| | (3-90-3) | | | | | | 0.60) |
| Example (3-247) | Compound | 5.0 | 13.9 | 5000.0 | 35.9 | 119.4 | (0.33, |
| | (3-91-3) | | | | | | 0.61) |
| Example (3-248) | Compound | 4.6 | 14.3 | 5000.0 | 35.1 | 106.6 | (0.30, |
| | (3-92-3) | | | | | | 0.60) |
| Example (3-249) | Compound | 4.7 | 14.3 | 5000.0 | 35.1 | 111.3 | (0.31, |
| | (3-93-3) | | | | | | 0.61) |
| Example (3-250) | Compound | 4.5 | 13.9 | 5000.0 | 35.9 | 98.8 | (0.31, |
| | (3-94-3) | | | | | | 0.60) |
| Example (3-251) | Compound | 4.9 | 13.9 | 5000.0 | 35.9 | 129.9 | (0.33, |
| | (3-95-3) | | | | | | 0.61) |
| Example (3-252) | Compound | 4.6 | 13.9 | 5000.0 | 36.0 | 146.9 | (0.32, |
| | (3-96-3) | | | | | | 0.61) |
| Example (3-253) | Compound | 4.8 | 14.2 | 5000.0 | 35.2 | 123.0 | (0.33, |
| | (3-97-3) | | | | | | 0.61) |
| Example (3-254) | Compound | 4.7 | 14.3 | 5000.0 | 35.1 | 91.8 | (0.30, |
| | (3-98-3) | | | | | | 0.60) |
| Example (3-255) | Compound | 5.0 | 13.5 | 5000.0 | 36.9 | 93.4 | (0.32, |
| | (3-99-3) | | | | | | 0.61) |
| Example (3-256) | Compound | 4.7 | 14.1 | 5000.0 | 35.5 | 102.4 | (0.31, |
| | (3-100-3) | | | | | | 0.60) |
| Example (3-257) | Compound | 5.0 | 13.7 | 5000.0 | 36.5 | 136.5 | (0.30, |
| | (3-101-3) | | | | | | 0.60) |
| Example (3-258) | Compound | 4.8 | 13.7 | 5000.0 | 36.5 | 90.8 | (0.31, |
| | (3-102-3) | | | | | | 0.61) |
| Example (3-259) | Compound | 4.9 | 14.2 | 5000.0 | 35.2 | 130.2 | (0.31, |
| | (3-103-3) | | | | | | 0.60) |
| Example (3-260) | Compound | 4.5 | 13.9 | 5000.0 | 35.9 | 100.4 | (0.33, |
| | (3-104-3) | | | | | | 0.61) |
| Example (3-261) | Compound | 4.6 | 13.7 | 5000.0 | 36.5 | 103.5 | (0.32, |
| | (3-105-3) | | | | | | 0.61) |
| Example (3-262) | Compound | 4.7 | 13.6 | 5000.0 | 36.7 | 100.7 | (0.33, |
| | (3-106-3) | | | | | | 0.60) |
| Example (3-263) | Compound | 5.0 | 13.8 | 5000.0 | 36.1 | 101.6 | (0.32, |
| | (3-107-3-3) | | | | | | 0.61) |
| Example (3-264) | Compound | 4.9 | 13.7 | 5000.0 | 36.5 | 148.8 | (0.31, |
| | (3-108-3) | | | | | | 0.60) |
| Example (3-265) | Compound | 4.7 | 13.7 | 5000.0 | 36.6 | 128.6 | (0.31, |
| | (3-109-3) | | | | | | 0.61) |
| Example (3-266) | Compound | 4.8 | 13.8 | 5000.0 | 36.3 | 107.2 | (0.31, |
| | (3-110-3) | | | | | | 0.60) |
| Example (3-267) | Compound | 4.6 | 13.6 | 5000.0 | 36.9 | 106.4 | (0.33, |
| | (3-111-3) | | | | | | 0.61) |
| Example (3-268) | Compound | 4.9 | 13.7 | 5000.0 | 36.6 | 129.4 | (0.30, |
| | (3-112-3) | | | | | | 0.60) |
| Example (3-269) | Compound | 4.6 | 14.0 | 5000.0 | 35.6 | 118.5 | (0.31, |
| | (3-113-3) | | | | | | 0.61) |
| Example (3-270) | Compound | 4.6 | 13.9 | 5000.0 | 36.0 | 124.6 | (0.31, |
| | (3-114-3) | | | | | | 0.60) |
| Example (3-271) | Compound | 4.7 | 13.6 | 5000.0 | 36.8 | 148.8 | (0.33, |
| | (3-115-3) | | | | | | 0.61) |
| Example (3-272) | Compound | 4.5 | 14.1 | 5000.0 | 35.5 | 138.6 | (0.32, |
| | (3-116-3) | | | | | | 0.61) |
| Example (3-273) | Compound | 4.7 | 13.8 | 5000.0 | 36.2 | 113.9 | (0.33, |
| | (3-117-3) | | | | | | 0.60) |
| Example (3-274) | Compound | 4.8 | 13.9 | 5000.0 | 35.9 | 94.3 | (0.32, |
| | (3-118-3) | | | | | | 0.61) |
| Example (3-275) | Compound | 4.9 | 13.6 | 5000.0 | 36.8 | 123.1 | (0.31, |
| | (3-119-3) | | | | | | 0.60) |
| Example (3-276) | Compound | 5.0 | 13.9 | 5000.0 | 36.0 | 117.1 | (0.31, |
| | (3-120-3) | | | | | | 0.61) |
| Example (3-277) | Compound | 4.6 | 13.6 | 5000.0 | 36.8 | 105.1 | (0.31, |
| | (3-121-3) | | | | | | 0.60) |
| Example (3-278) | Compound | 4.7 | 13.7 | 5000.0 | 36.5 | 121.3 | (0.33, |
| | (3-122-3) | | | | | | 0.61) |
| Example (3-279) | Compound | 5.0 | 14.0 | 5000.0 | 35.6 | 116.1 | (0.30, |
| | (3-123-3) | | | | | | 0.60) |
| Example (3-280) | Compound | 4.9 | 13.6 | 5000.0 | 36.7 | 138.6 | (0.31, |
| | (3-124-3) | | | | | | 0.61) |
| Example (3-281) | Compound | 4.6 | 14.2 | 5000.0 | 35.1 | 135.4 | (0.31, |
| | (3-125-3) | | | | | | 0.60) |
| Example (3-282) | Compound | 4.9 | 13.8 | 5000.0 | 36.2 | 134.8 | (0.33, |
| | (3-126-3) | | | | | | 0.61) |
| Example (3-283) | Compound | 4.6 | 13.7 | 5000.0 | 36.5 | 94.1 | (0.32, |
| | (3-127-3) | | | | | | 0.61) |
| Example (3-284) | Compound | 4.7 | 14.2 | 5000.0 | 35.2 | 128.7 | (0.33, |
| | (3-128-3) | | | | | | 0.60) |
| Example (3-285) | Compound | 4.8 | 15.2 | 5000.0 | 32.8 | 104.7 | (0.31, |
| | (4-1-3) | | | | | | 0.61) |
| Example (3-286) | Compound | 4.8 | 15.0 | 5000.0 | 33.4 | 140.9 | (0.31, |
| | (4-2-3) | | | | | | 0.60) |
| Example (3-287) | Compound | 4.7 | 15.1 | 5000.0 | 33.0 | 114.5 | (0.33, |
| | (4-3-3) | | | | | | 0.61) |
| Example (3-288) | Compound | 4.6 | 15.5 | 5000.0 | 32.3 | 98.0 | (0.32, |
| | (4-4-3) | | | | | | 0.61) |
| Example (3-289) | Compound | 4.9 | 15.9 | 5000.0 | 31.4 | 135.6 | (0.33, |
| | (4-5-3) | | | | | | 0.60) |
| Example (3-290) | Compound | 4.8 | 15.0 | 5000.0 | 33.4 | 140.9 | (0.32, |
| | (4-6-3) | | | | | | 0.61) |
| Example (3-291) | Compound | 4.5 | 16.2 | 5000.0 | 30.9 | 142.3 | (0.31, |
| | (4-7-3) | | | | | | 0.60) |
| Example (3-292) | Compound | 4.9 | 16.1 | 5000.0 | 31.1 | 107.7 | (0.31, |
| | (4-8-3) | | | | | | 0.61) |
| Example (3-293) | Compound | 4.8 | 15.9 | 5000.0 | 31.5 | 116.8 | (0.31, |
| | (4-9-3) | | | | | | 0.60) |
| Example (3-294) | Compound | 4.9 | 16.2 | 5000.0 | 30.8 | 145.8 | (0.33, |
| | (4-10-3) | | | | | | 0.61) |
| Example (3-295) | Compound | 4.8 | 14.9 | 5000.0 | 33.6 | 124.2 | (0.30, |
| | (4-11-3) | | | | | | 0.60) |
| Example (3-296) | Compound | 4.8 | 16.4 | 5000.0 | 30.5 | 111.0 | (0.31, |
| | (4-12-3) | | | | | | 0.61) |
| Example (3-297) | Compound | 4.7 | 14.3 | 5000.0 | 34.9 | 142.5 | (0.31, |
| | (4-13-3) | | | | | | 0.60) |
| Example (3-298) | Compound | 4.7 | 15.2 | 5000.0 | 32.9 | 125.8 | (0.33, |
| | (4-14-3) | | | | | | 0.61) |
| Example (3-299) | Compound | 4.8 | 14.9 | 5000.0 | 33.5 | 97.6 | (0.32, |
| | (4-15-3) | | | | | | 0.61) |
| Example (3-300) | Compound | 4.8 | 15.5 | 5000.0 | 32.3 | 128.8 | (0.33, |
| | (4-16-3) | | | | | | 0.60) |
| Example (3-301) | Compound | 5.0 | 16.6 | 5000.0 | 30.2 | 133.7 | (0.32, |
| | (4-17-3) | | | | | | 0.61) |
| Example (3-302) | Compound | 4.7 | 14.6 | 5000.0 | 34.3 | 98.7 | (0.31, |
| | (4-18-3) | | | | | | 0.60) |
| Example (3-303) | Compound | 5.0 | 16.6 | 5000.0 | 30.1 | 144.7 | (0.31, |
| | (4-19-3) | | | | | | 0.61) |
| Example (3-304) | Compound | 4.7 | 16.6 | 5000.0 | 30.1 | 122.4 | (0.31, |
| | (4-20-3) | | | | | | 0.60) |
| Example (3-305) | Compound | 4.7 | 16.2 | 5000.0 | 30.9 | 147.1 | (0.33, |
| | (4-21-3) | | | | | | 0.61) |
| Example (3-306) | Compound | 4.6 | 16.1 | 5000.0 | 31.1 | 114.7 | (0.30, |
| | (4-22-3) | | | | | | 0.60) |
| Example (3-307) | Compound | 4.5 | 16.3 | 5000.0 | 30.6 | 125.7 | (0.31, |
| | (4-23-3) | | | | | | 0.61) |
| Example (3-308) | Compound | 4.8 | 16.5 | 5000.0 | 30.4 | 114.9 | (0.31, |
| | (4-24-3) | | | | | | 0.60) |
| Example (3-309) | Compound | 4.9 | 16.3 | 5000.0 | 30.7 | 124.3 | (0.33, |
| | (4-25-3) | | | | | | 0.61) |
| Example (3-310) | Compound | 4.5 | 15.3 | 5000.0 | 32.7 | 92.8 | (0.32, |
| | (4-26-3) | | | | | | 0.61) |
| Example (3-311) | Compound | 4.5 | 14.8 | 5000.0 | 33.8 | 141.5 | (0.33, |
| | (4-27-3) | | | | | | 0.60) |
| Example (3-312) | Compound | 5.0 | 15.2 | 5000.0 | 32.9 | 108.3 | (0.32, |
| | (4-28-3) | | | | | | 0.61) |

### II. Manufacture and Test of Red Organic Electronic Light Emitting Element (Phosphorescent Host)

### Example 3-313 Red Organic Light Emitting Element (Phosphorescent Host)

An organic electronic light emitting element was manufactured by an ordinary method using the compound obtained through synthesis as a light emitting host material for a light emitting layer. First, a film of N¹-(naphthalen-2-yl)-N⁴,N⁴-bis(4-(naphthalen-2-yl(phenyl)amino)phenyl)-N¹-phenylbenzene-1,4-diamine (hereinafter, abbreviated as “2-TNATA”) as a hole transport compound was vacuum-deposited on an ITO layer (anode) formed on a galas substrate to form a hole injection layer with a thickness 60 nm, and then, 4,4-bis[N-(1-naphthyl)-N-phenylamino]biphenyl (hereinafter, abbreviated as “-NPD”) as a hole transport compound was vacuum-deposited on the hole injection layer to form a hole transport layer with a thickness of 60 nm. Then, a light emitting layer with a thickness of 30 nm was deposited on the hole transport layer by doping an upper portion of the hole transport layer with compound 2-41-3 of the present invention as a host material and (piq)₂Ir(acac) [bis-(1-phenylisoquinolyl)iridium(III)acetylacetonate] as a dopant material at a weight ratio of 95:5. Then, (1.1′-bisphenyl)-4-olato)bis(2-methyl-8-quinolinolato)aluminum (hereinafter, abbreviated as “BAlq”) was vacuum-deposited with a thickness of 10 nm for a hole blocking layer, and tris(8-quinolinol)aluminum (hereinafter, abbreviated as “Alq3”) was formed with a thickness of 40 nm for an electron transport layer. Thereafter, LiF as halogenated alkali metal was deposited with a thickness of 0.2 nm for an electron injection layer, and then Al was deposited with a thickness of 150 nm to be used as a cathode. In this way, an organic electronic light emitting element was manufactured.

### Example 3-314 to Example 3-336 Red Organic Electronic Light Emitting Element (Phosphorescent Host)

An organic electronic light emitting element was manufactured by the same method as in Example 3-313 except that, instead of compound 2-41-3 of the present invention, one of compounds 2-42-3 to 2-52-3 and 3-41-3 to 3-52-3 listed on table 6 was used as a phosphorescent host material for a light emitting layer.

### Comparative Example 3-5

An organic electronic light emitting element was manufactured by the same method as in Example 3-313 except that, instead of compound 2-41-3 of the present invention, comparative compound A [4,4′-N,N′-dicarbazole-biphenyl (CBP)] above was used as a phosphorescent host material for a light emitting layer.

### Comparative Example 3-6

An organic electronic light emitting element was manufactured by the same method as in Example 3-313 except that, instead of compound 2-41-3 of the present invention, comparative compound B above was used as a phosphorescent host material for a light emitting layer.

### Comparative Example 3-7

An organic electronic light emitting element was manufactured by the same method as in Example 3-313 except that, instead of compound 2-41-3 of the present invention, comparative compound C above was used as a phosphorescent host material for a light emitting layer.

### Comparative Example 3-8

An organic electronic light emitting element was manufactured by the same method as in Example 3-313 except that, instead of compound 2-41-3 of the present invention, comparative compound D above was used as a phosphorescent host material for a light emitting layer.

A forward bias DC voltage was applied to the organic electronic light emitting elements manufactured in the examples and the comparative examples to measure electro-luminescence (EL) characteristics thereof by PR-650 (Photoresearch), and the T95 lifetime was measured by lifetime measuring equipments (Mcscience) at reference brightness of 2500 cd/m². Table 3-6 below shows the manufacture of elements and evaluation results thereof.

TABLE 3-6

| | | | Current | Brightness | | Lifetime | CIE |
|---|---|---|---|---|---|---|---|
| | Compound | Voltage | Density | (cd/m2) | Efficiency | T(95) | (x, y) |
| Comparative | Compound | 6.2 | 39.7 | 2500.0 | 6.3 | 53.3 | (0.31, |
| Example (3-5) | (A) | | | | | | 0.60) |
| Comparative | Compound | 5.7 | 32.5 | 2500.0 | 7.7 | 97.2 | (0.31, |
| Example (3-6) | (B) | | | | | | 0.61) |
| Comparative | Compound | 5.8 | 34.8 | 2500.0 | 7.2 | 91.8 | (0.31, |
| Example (3-7) | (C) | | | | | | 0.60) |
| Comparative | Compound | 5.9 | 34.7 | 2500.0 | 7.2 | 93.3 | (0.33, |
| Example (3-8) | (D) | | | | | | 0.61) |
| Example (3-313) | Compound | 5.2 | 25.6 | 2500.0 | 9.8 | 146.6 | (0.30, |
| | (2-41-3) | | | | | | 0.60) |
| Example (3-314) | Compound | 5.3 | 26.1 | 2500.0 | 9.6 | 101.1 | (0.31, |
| | (2-42-3) | | | | | | 0.61) |
| Example (3-315) | Compound | 5.4 | 30.0 | 2500.0 | 8.3 | 133.9 | (0.31, |
| | (2-43-3) | | | | | | 0.60) |
| Example (3-316) | Compound | 5.2 | 26.7 | 2500.0 | 9.4 | 112.3 | (0.33, |
| | (2-44-3) | | | | | | 0.61) |
| Example (3-317) | Compound | 5.3 | 27.4 | 2500.0 | 9.1 | 99.8 | (0.32, |
| | (2-45-3) | | | | | | 0.61) |
| Example (3-318) | Compound | 5.3 | 27.3 | 2500.0 | 9.2 | 124.4 | (0.33, |
| | (2-46-3) | | | | | | 0.60) |
| Example (3-319) | Compound | 5.1 | 29.8 | 2500.0 | 8.4 | 116.6 | (0.32, |
| | (2-47-3) | | | | | | 0.61) |
| Example (3-320) | Compound | 5.1 | 25.8 | 2500.0 | 9.7 | 146.3 | (0.31, |
| | (2-48-3) | | | | | | 0.60) |
| Example (3-321) | Compound | 5.1 | 29.1 | 2500.0 | 8.6 | 141.1 | (0.31, |
| | (2-49-3) | | | | | | 0.61) |
| Example (3-322) | Compound | 5.3 | 27.8 | 2500.0 | 9.0 | 111.0 | (0.31, |
| | (2-50-3) | | | | | | 0.60) |
| Example (3-323) | Compound | 5.1 | 28.6 | 2500.0 | 8.7 | 135.1 | (0.33, |
| | (2-51-3) | | | | | | 0.61) |
| Example (3-324) | Compound | 5.4 | 30.4 | 2500.0 | 8.2 | 122.5 | (0.30, |
| | (2-52-3) | | | | | | 0.60) |
| Example (3-325) | Compound | 5.3 | 26.6 | 2500.0 | 9.4 | 144.0 | (0.31, |
| | (3-41-3) | | | | | | 0.61) |
| Example (3-326) | Compound | 5.1 | 29.5 | 2500.0 | 8.5 | 120.4 | (0.31, |
| | (3-42-3) | | | | | | 0.60) |
| Example (3-327) | Compound | 5.2 | 30.2 | 2500.0 | 8.3 | 123.6 | (0.33, |
| | (3-43-3) | | | | | | 0.61) |
| Example (3-328) | Compound | 5.3 | 29.8 | 2500.0 | 8.4 | 141.3 | (0.32, |
| | (3-44-3) | | | | | | 0.61) |
| Example (3-329) | Compound | 5.1 | 28.2 | 2500.0 | 8.9 | 128.9 | (0.33, |
| | (3-45-3) | | | | | | 0.60) |
| Example (3-330) | Compound | 5.3 | 28.2 | 2500.0 | 8.9 | 119.7 | (0.31, |
| | (3-46-3) | | | | | | 0.60) |
| Example (3-331) | Compound | 5.1 | 28.5 | 2500.0 | 8.8 | 98.0 | (0.31, |
| | (3-47-3) | | | | | | 0.61) |
| Example (3-332) | Compound | 5.2 | 29.5 | 2500.0 | 8.5 | 116.6 | (0.31, |
| | (3-48-3) | | | | | | 0.60) |
| Example (3-333) | Compound | 5.2 | 29.4 | 2500.0 | 8.5 | 100.2 | (0.33, |
| | (3-49-3) | | | | | | 0.61) |
| Example (3-334) | Compound | 5.3 | 25.5 | 2500.0 | 9.8 | 134.3 | (0.30, |
| | (3-50-3) | | | | | | 0.60) |
| Example (3-335) | Compound | 5.3 | 29.1 | 2500.0 | 8.6 | 113.9 | (0.31, |
| | (3-51-3) | | | | | | 0.61) |
| Example (3-336) | Compound | 5.1 | 25.9 | 2500.0 | 9.7 | 105.9 | (0.31, |
| | (3-52-3) | | | | | | 0.60) |

As can be seen from the results on table 3-5 and table 3-6, the organic electronic light emitting elements using the materials for the organic electronic light emitting element of the present invention as a phosphorescent host showed a low driving voltage, high light emitting efficiency, and a long lifetime.

In other words, comparative compounds B, C, and D having bis-carbazole as a core showed excellent element results compared with comparative compound A, which is CBP generally used as a host material, and the compounds of the present invention having carbazole linked to carboline showed the best results in view of a driving voltage, efficiency, and a lifetime, compared with comparative compounds B, C, and D.

The compound according to the present invention has a bipolar since it is composed of carbazole and carboline. Therefore, it is considered that the compounds of the present invention can raise the charge balance in the light emitting layer compared with those in comparative compounds B, C, and D, leading to an increase in efficiency, and shows less hole accumulation in the light emitting layer compared with comparative compounds B, C, and D, leading to a long lifetime (In the driving of OLED, holes generally have 1000-fold higher mobility than electrons).

In addition, the compounds according to the present invention have similar T1 values to comparative compounds B, C, and D, but show lower LUMO values, and resultantly, it is considered that the compounds of the present invention may easily receive electrons from the electron transport layer, leading to a low driving voltage and excellent thermal stability (thermal damage due to a high driving voltage).

In addition, the characteristics of elements have been described in view of a light emitting layer from the foregoing evaluation results of the manufacture of elements, but the materials ordinarily used for a light emitting layer may be used alone or in a mixture with other materials, for the foregoing organic material layer for an organic electronic element, such as an electron transport layer, an electron injection layer, a hole injection layer, a hole transport layer, and an auxiliary light emitting layer. Therefore, for the foregoing reasons, the compounds of the present invention may be used alone or in a mixture with other materials, for the other layers for the organic material layer excluding the light emitting layer, for example, an electron transport layer, an electron injection layer, a hole injection layer, a hole transport layer, and an auxiliary light emitting layer.

### Example 4

The compound according to an aspect of the present invention is represented by Formula 4-1 below.

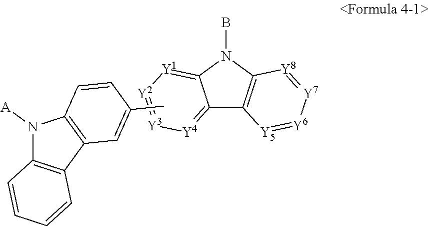

In Formula 4-1,

A and B each may be independently selected from the group consisting of a C₆-C₆₀ aryl group, a fluorenyl group, a C₂-C₆₀ heterocyclic group containing at least one heteroatom of O, N, S, Si, and P, a fused ring group of a C₃-C₆₀ aliphatic group and a C₆-C₆₀ aromatic group, a C₁-C₅₀ alkyl group, a C₂-C₂₀ alkenyl group, a C₂-C₂₀ alkynyl group, C₁-C₃₀ alkoxyl group, a C₆-C₃₀ aryloxy group, and -L′-N(Rₐ) (R_{b}).

L′ may be selected from the group consisting of a single bond, a C₆-C₆₀ arylene group, a fluorenyl group, a fused ring group of a C₃-C₆₀ aliphatic group and a C₆-C₆₀ aromatic group, and a C₂-C₆₀ heterocyclic group.

Rₐ and R_{b} each may be independently selected from the group consisting of a C₆-C₆₀ aryl group, a fluorenylene group, a fused ring group of a C₃-C₆₀ aliphatic group and a C₆-C₆₀ aromatic group, and a C₂-C₆o heterocyclic group containing at least one heteroatom of O, N, S, Si, and P.

For example, when A, B, L′, Rₐ, and R_{b} are an aryl group, A, B, L′, Rₐ, and R_{b} each may be independently a phenyl group, a biphenyl group, a naphthyl group, or the like.

Y₁ to Y₈ each may be independently CR or N, and at least one of Y₁ to Y₈ may be N.

At least one of R's may be linked to carbazole substituted with A, and R that is not linked thereto may be hydrogen.

However, the compounds wherein one of carbazoles substituted with A is linked to Y₃, and only Y₈ among Y₁, Y₂, and Y₄-Y₈ is N are excluded.

the aryl group, fluorenyl group, heterocyclic group, fused ring group, alkyl group, alkenyl group, alkoxyl group, aryloxy group, arylene group, and fluorenylene group each may be substituted with at least one substituent selected from the group consisting of deuterium, halogen, a silane group, a siloxane group, a boron group, a germanium group, a cyano group, a nitro group, a C₁-C₂₀ alkylthio group, a C₁-C₂₀ alkoxyl group, a C₁-C₂₀ alkyl group, a C₂-C₂₀ alkenyl group, a C₂-C₂₀ alkynyl group, a C₆-C₂₀ aryl group, a C₆-C₂₀ aryl group substituted with deuterium, a fluorenyl group, a C₂-C₂₀ heterocyclic group, a C₃-C₂₀ cycloalkyl group, a C₇-C₂₀ arylalkyl group, and a C₈-C₂₀ arylalkenyl group.

Here, the aryl group may be an aryl group having 6-60 carbon atoms, preferably 6-40 carbon atoms, and more preferably 6-30 carbon atoms;

the heterocyclic group may be a heterocyclic group having 2-60 carbon atoms, preferably 2-30 carbon atoms, and more preferably 2-20 carbon atoms;

the arylene group may be an arylene group having 6-60 carbon atoms, preferably 6-30 carbon atoms, and more preferably 6-20 carbon atoms; and

the alkyl group may be an alkyl group having 1-50 carbon atoms, preferably 1-30 carbon atoms, more preferably 1-20 carbon atoms, and especially preferably 1-10 carbon atoms.

Specifically, the compound represented by Formula 4-1 above may be expressed by one of the following compounds.

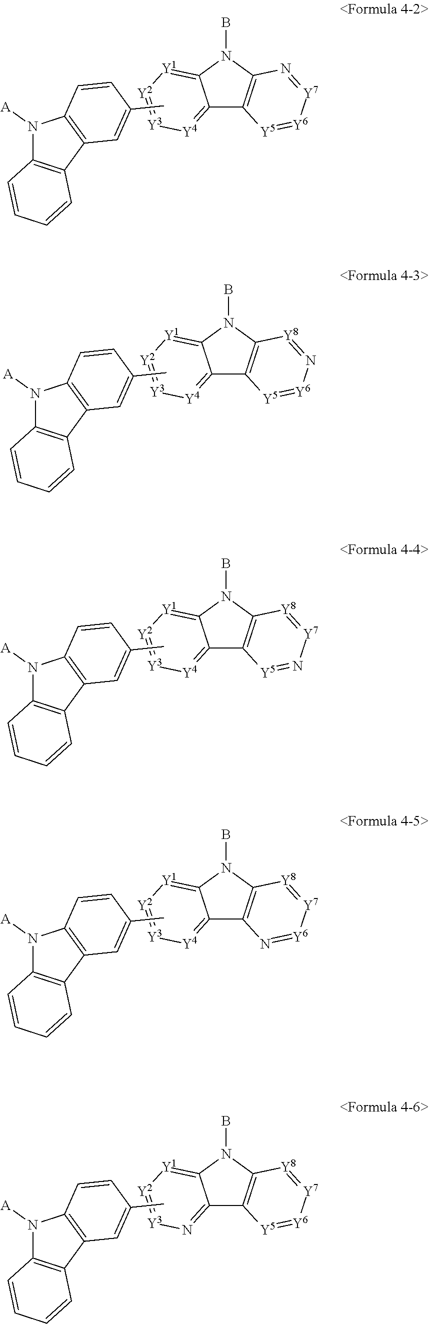

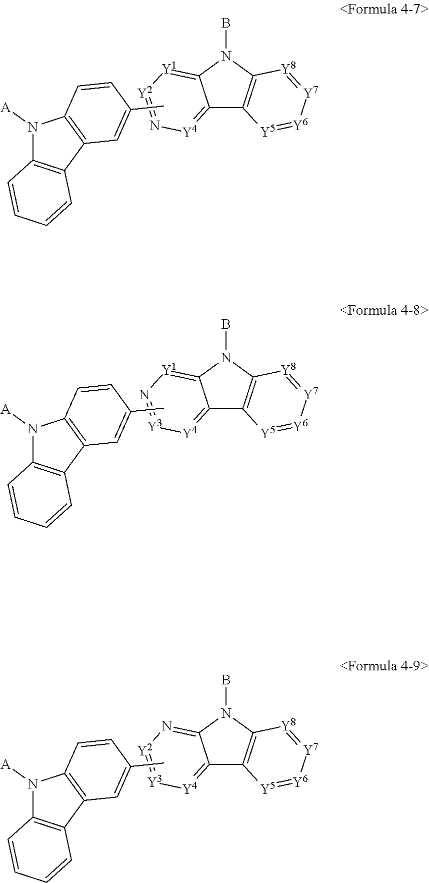

In Formulas 4-2 to 4-9,

Y₁ to Y₈ and A and B may be identical Y₁ to Y₈ and A and B defined in Formula 4-1. However, in Formula 4-2,

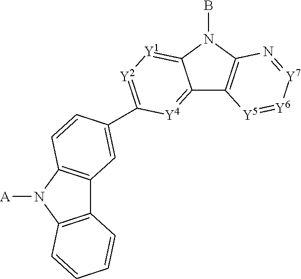

is excluded.

More specifically, the compounds represented by Formula 4-1 may be one of the following compounds.

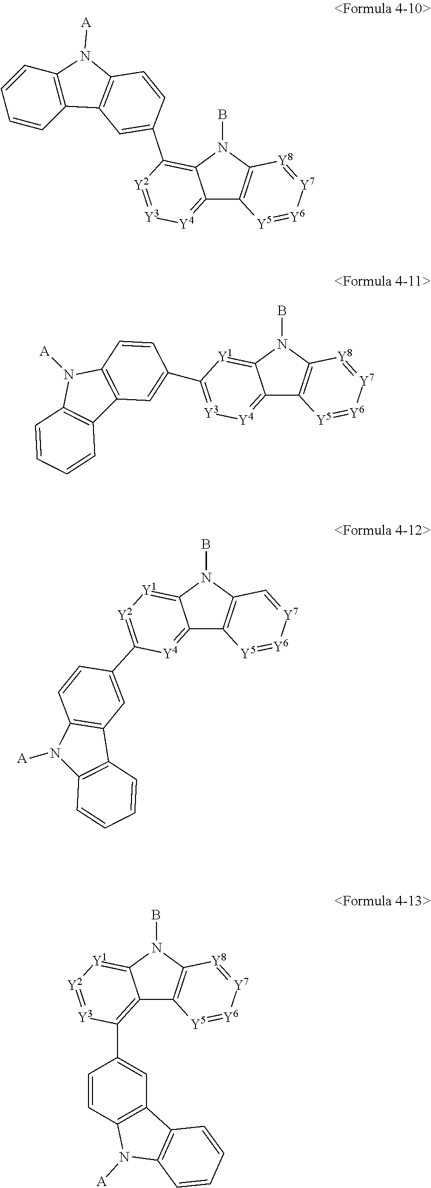

In Formulas 4-10 to 4-13, Y₁ to Y₈ each may be independently CH or N, and at least one of Y₁ to Y₈ is N, and A and B may be identical A and B defined in Formula 4-1.

More specifically, the compounds represented by Formulas 4-1 to 4-13 may be one of the following compounds.

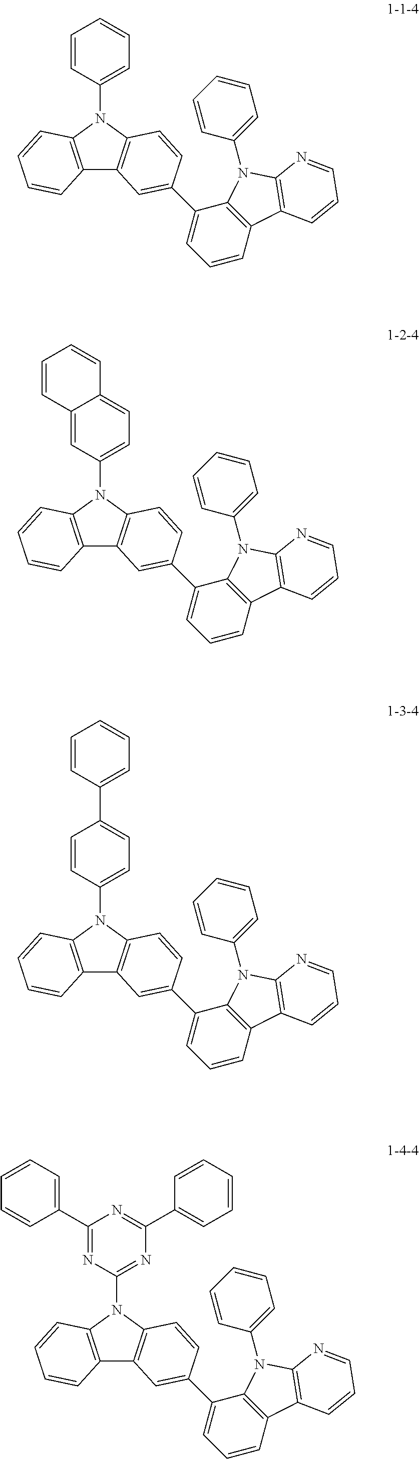 [SMILES: c1ccc(-n2c3ccccc3c3cc(-c4cccc5c6cccnc6n(-c6ccccc6)c45)ccc32)cc1 ; c1ccc(-n2c3ncccc3c3cccc(-c4ccc5c(c4)c4ccccc4n5-c4ccc5ccccc5c4)c32)cc1 ; c1ccc(-c2ccc(-n3c4ccccc4c4cc(-c5cccc6c7cccnc7n(-c7ccccc7)c56)ccc43)cc2)cc1 ; c1ccc(-c2nc(-c3ccccc3)nc(-n3c4ccccc4c4cc(-c5cccc6c7cccnc7n(-c7ccccc7)c56)ccc43)n2)cc1]

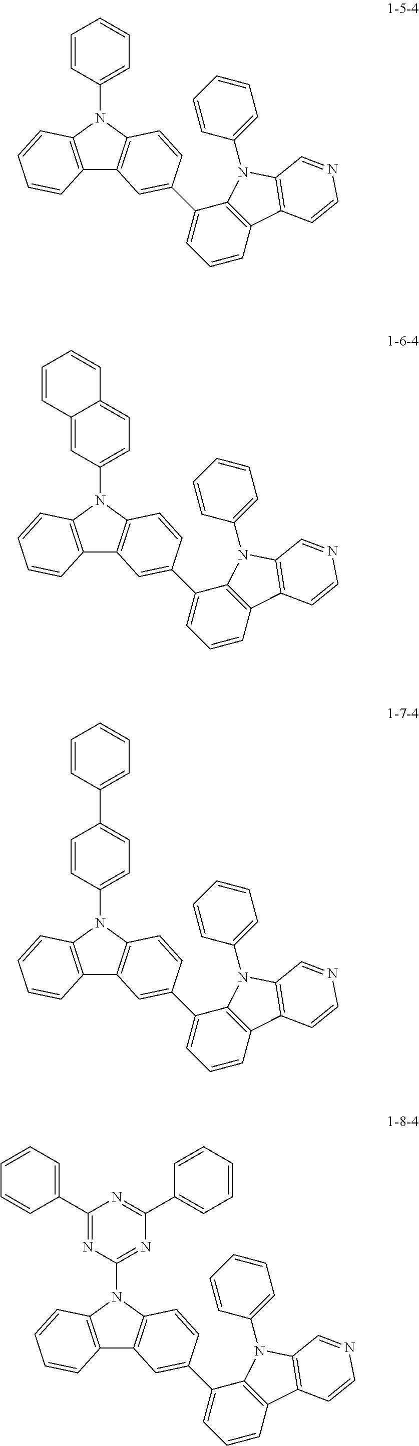 [SMILES: c1ccc(-n2c3ccccc3c3cc(-c4cccc5c6ccncc6n(-c6ccccc6)c45)ccc32)cc1 ; c1ccc(-n2c3cnccc3c3cccc(-c4ccc5c(c4)c4ccccc4n5-c4ccc5ccccc5c4)c32)cc1 ; c1ccc(-c2ccc(-n3c4ccccc4c4cc(-c5cccc6c7ccncc7n(-c7ccccc7)c56)ccc43)cc2)cc1 ; c1ccc(-c2nc(-c3ccccc3)nc(-n3c4ccccc4c4cc(-c5cccc6c7ccncc7n(-c7ccccc7)c56)ccc43)n2)cc1]

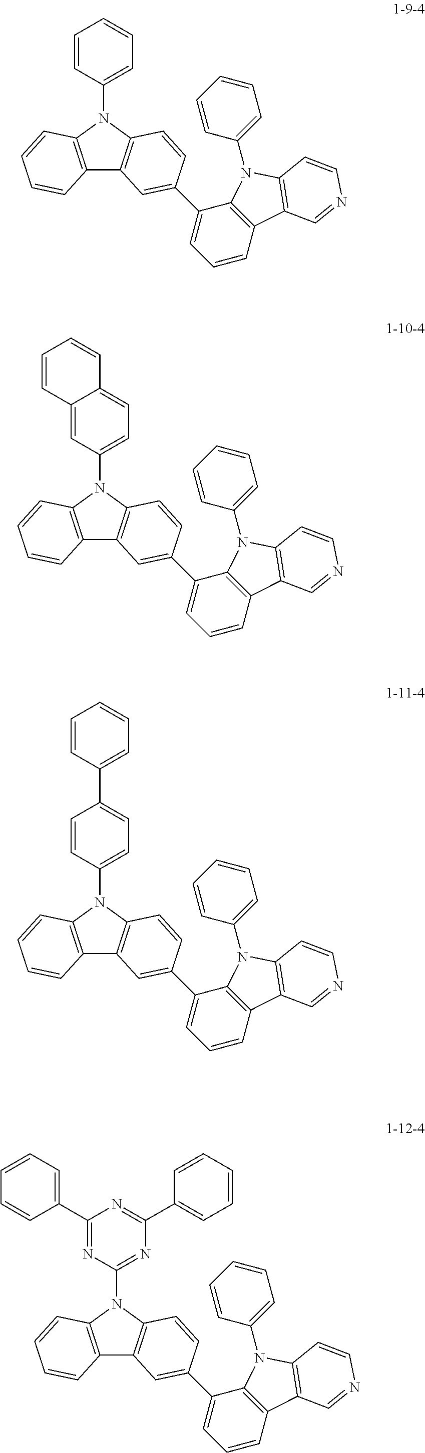 [SMILES: c1ccc(-n2c3ccccc3c3cc(-c4cccc5c6cnccc6n(-c6ccccc6)c45)ccc32)cc1 ; c1ccc(-n2c3ccncc3c3cccc(-c4ccc5c(c4)c4ccccc4n5-c4ccc5ccccc5c4)c32)cc1 ; c1ccc(-c2ccc(-n3c4ccccc4c4cc(-c5cccc6c7cnccc7n(-c7ccccc7)c56)ccc43)cc2)cc1 ; c1ccc(-c2nc(-c3ccccc3)nc(-n3c4ccccc4c4cc(-c5cccc6c7cnccc7n(-c7ccccc7)c56)ccc43)n2)cc1]

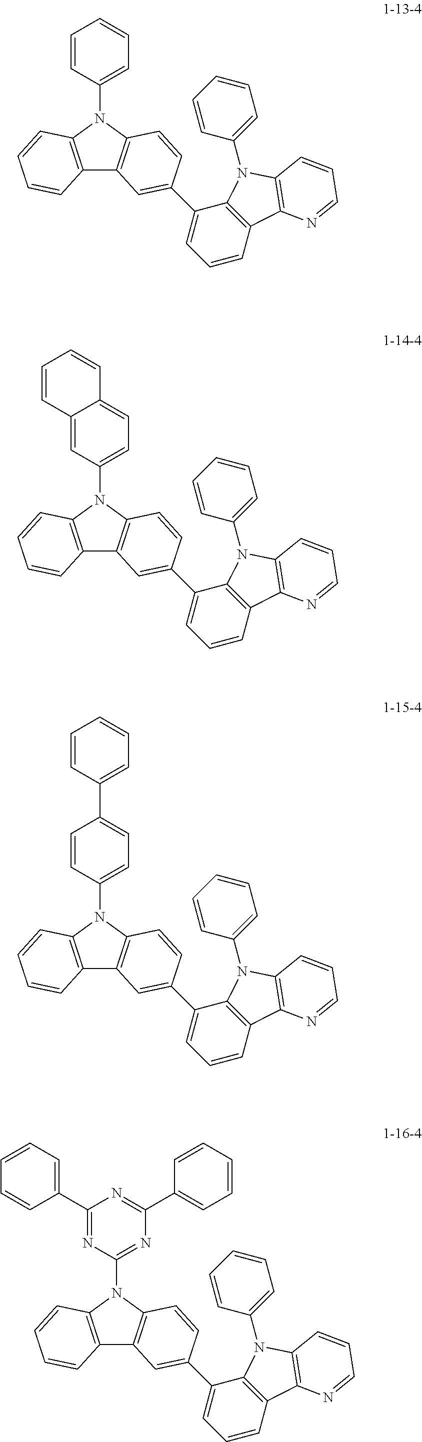 [SMILES: c1ccc(-n2c3ccccc3c3cc(-c4cccc5c6ncccc6n(-c6ccccc6)c45)ccc32)cc1 ; c1ccc(-n2c3cccnc3c3cccc(-c4ccc5c(c4)c4ccccc4n5-c4ccc5ccccc5c4)c32)cc1 ; c1ccc(-c2ccc(-n3c4ccccc4c4cc(-c5cccc6c7ncccc7n(-c7ccccc7)c56)ccc43)cc2)cc1 ; c1ccc(-c2nc(-c3ccccc3)nc(-n3c4ccccc4c4cc(-c5cccc6c7ncccc7n(-c7ccccc7)c56)ccc43)n2)cc1]

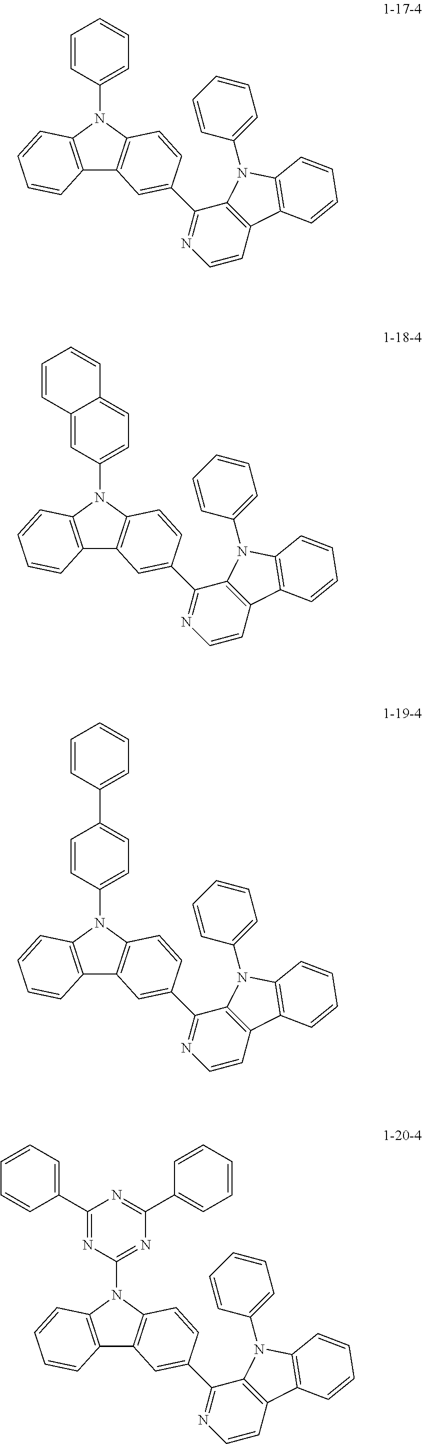 [SMILES: c1ccc(-n2c3ccccc3c3cc(-c4nccc5c6ccccc6n(-c6ccccc6)c45)ccc32)cc1 ; c1ccc(-n2c3ccccc3c3ccnc(-c4ccc5c(c4)c4ccccc4n5-c4ccc5ccccc5c4)c32)cc1 ; c1ccc(-c2ccc(-n3c4ccccc4c4cc(-c5nccc6c7ccccc7n(-c7ccccc7)c56)ccc43)cc2)cc1 ; c1ccc(-c2nc(-c3ccccc3)nc(-n3c4ccccc4c4cc(-c5nccc6c7ccccc7n(-c7ccccc7)c56)ccc43)n2)cc1]

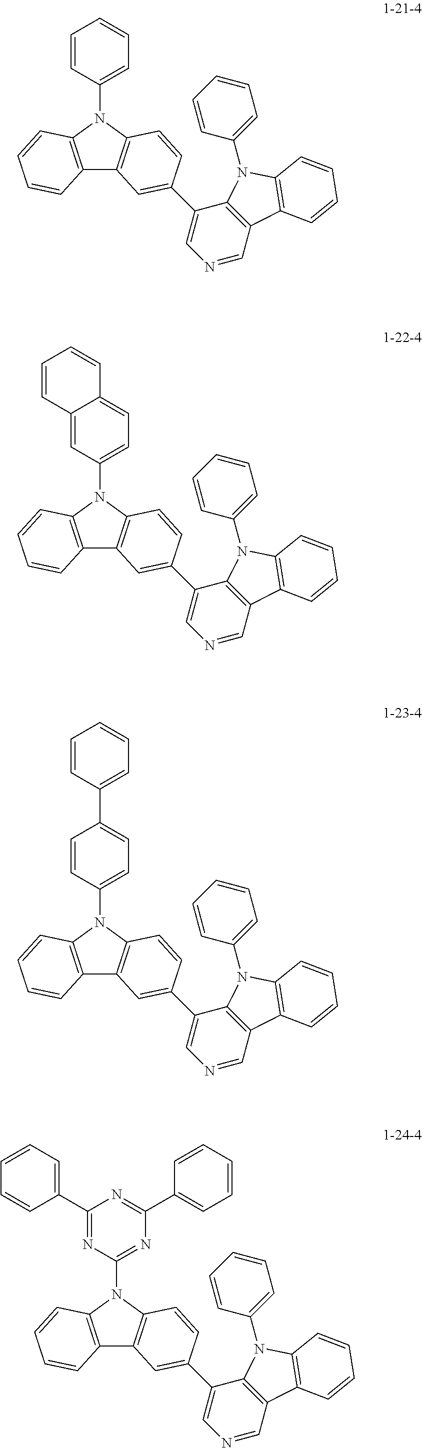 [SMILES: c1ccc(-n2c3ccccc3c3cc(-c4cncc5c6ccccc6n(-c6ccccc6)c45)ccc32)cc1 ; c1ccc(-n2c3ccccc3c3cncc(-c4ccc5c(c4)c4ccccc4n5-c4ccc5ccccc5c4)c32)cc1 ; c1ccc(-c2ccc(-n3c4ccccc4c4cc(-c5cncc6c7ccccc7n(-c7ccccc7)c56)ccc43)cc2)cc1 ; c1ccc(-c2nc(-c3ccccc3)nc(-n3c4ccccc4c4cc(-c5cncc6c7ccccc7n(-c7ccccc7)c56)ccc43)n2)cc1]

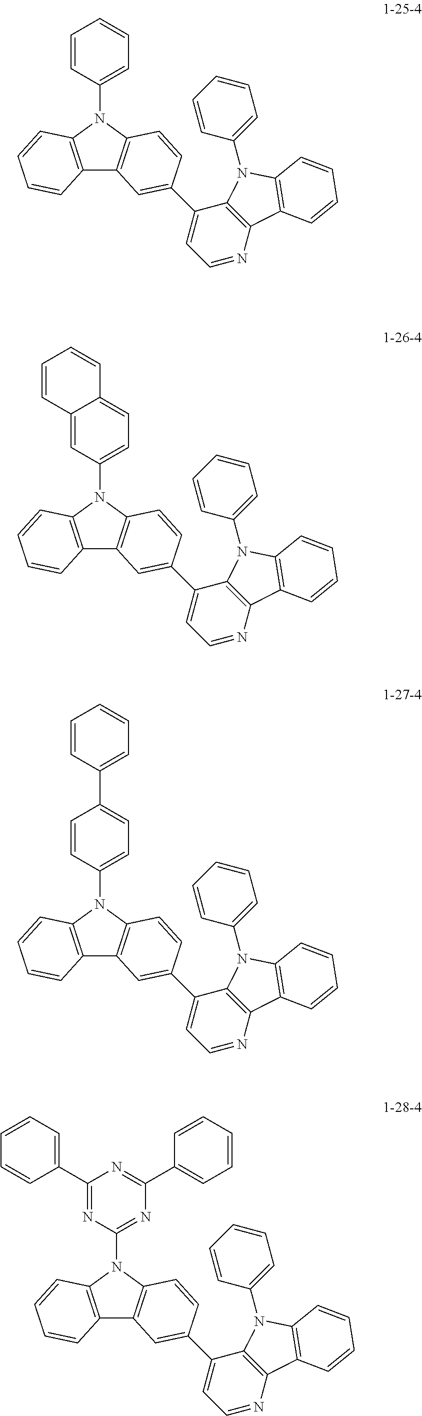 [SMILES: c1ccc(-n2c3ccccc3c3cc(-c4ccnc5c6ccccc6n(-c6ccccc6)c45)ccc32)cc1 ; c1ccc(-n2c3ccccc3c3nccc(-c4ccc5c(c4)c4ccccc4n5-c4ccc5ccccc5c4)c32)cc1 ; c1ccc(-c2ccc(-n3c4ccccc4c4cc(-c5ccnc6c7ccccc7n(-c7ccccc7)c56)ccc43)cc2)cc1 ; c1ccc(-c2nc(-c3ccccc3)nc(-n3c4ccccc4c4cc(-c5ccnc6c7ccccc7n(-c7ccccc7)c56)ccc43)n2)cc1]

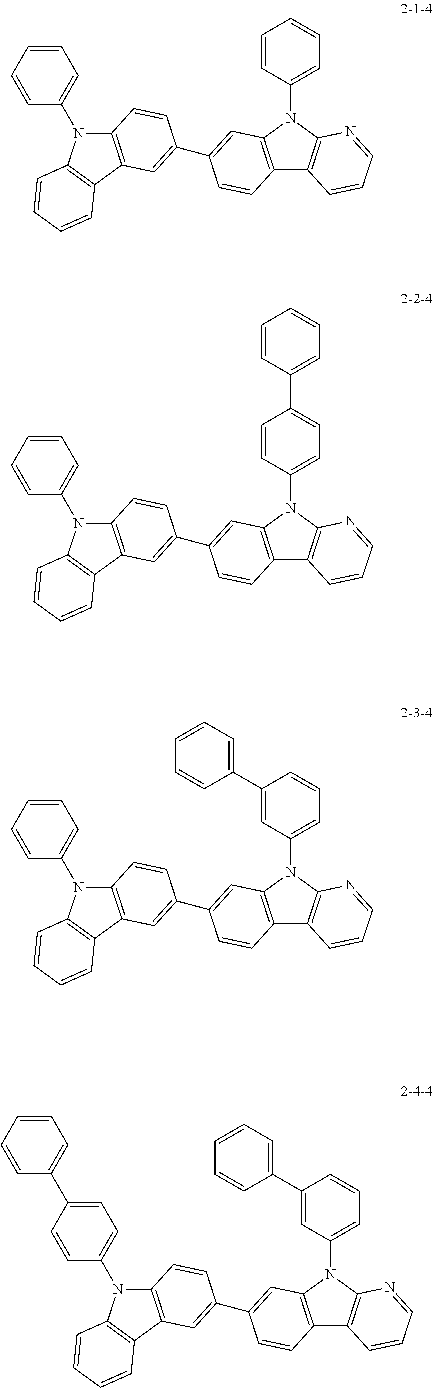 [SMILES: c1ccc(-n2c3ccccc3c3cc(-c4ccc5c6cccnc6n(-c6ccccc6)c5c4)ccc32)cc1 ; c1ccc(-c2ccc(-n3c4cc(-c5ccc6c(c5)c5ccccc5n6-c5ccccc5)ccc4c4cccnc43)cc2)cc1 ; c1ccc(-c2cccc(-n3c4cc(-c5ccc6c(c5)c5ccccc5n6-c5ccccc5)ccc4c4cccnc43)c2)cc1 ; c1ccc(-c2ccc(-n3c4ccccc4c4cc(-c5ccc6c7cccnc7n(-c7cccc(-c8ccccc8)c7)c6c5)ccc43)cc2)cc1]

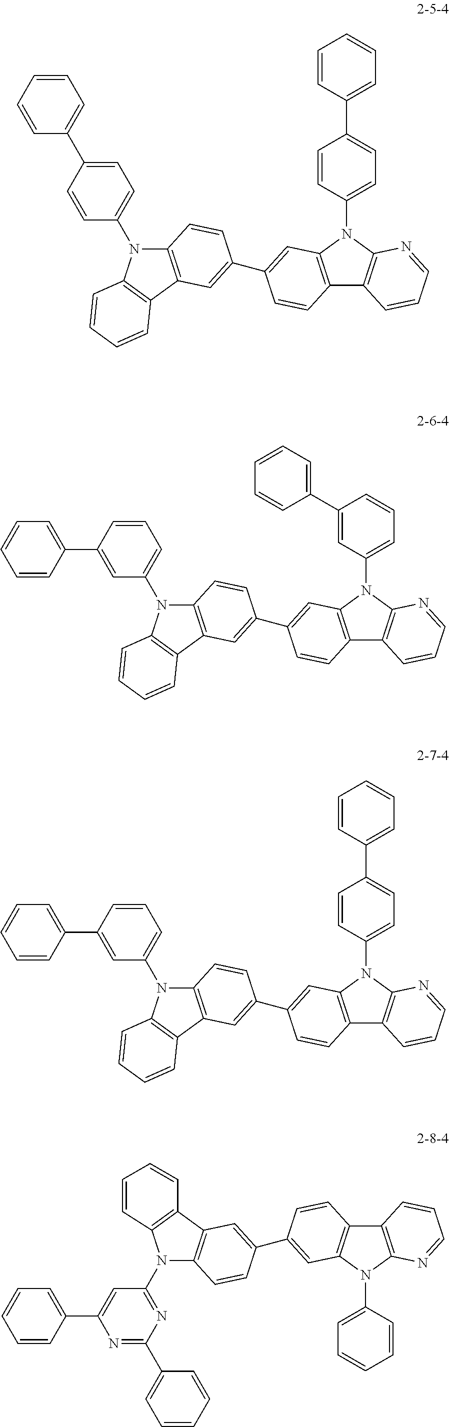 [SMILES: c1ccc(-c2ccc(-n3c4ccccc4c4cc(-c5ccc6c7cccnc7n(-c7ccc(-c8ccccc8)cc7)c6c5)ccc43)cc2)cc1 ; c1ccc(-c2cccc(-n3c4ccccc4c4cc(-c5ccc6c7cccnc7n(-c7cccc(-c8ccccc8)c7)c6c5)ccc43)c2)cc1 ; c1ccc(-c2ccc(-n3c4cc(-c5ccc6c(c5)c5ccccc5n6-c5cccc(-c6ccccc6)c5)ccc4c4cccnc43)cc2)cc1 ; c1ccc(-c2cc(-n3c4ccccc4c4cc(-c5ccc6c7cccnc7n(-c7ccccc7)c6c5)ccc43)nc(-c3ccccc3)n2)cc1]

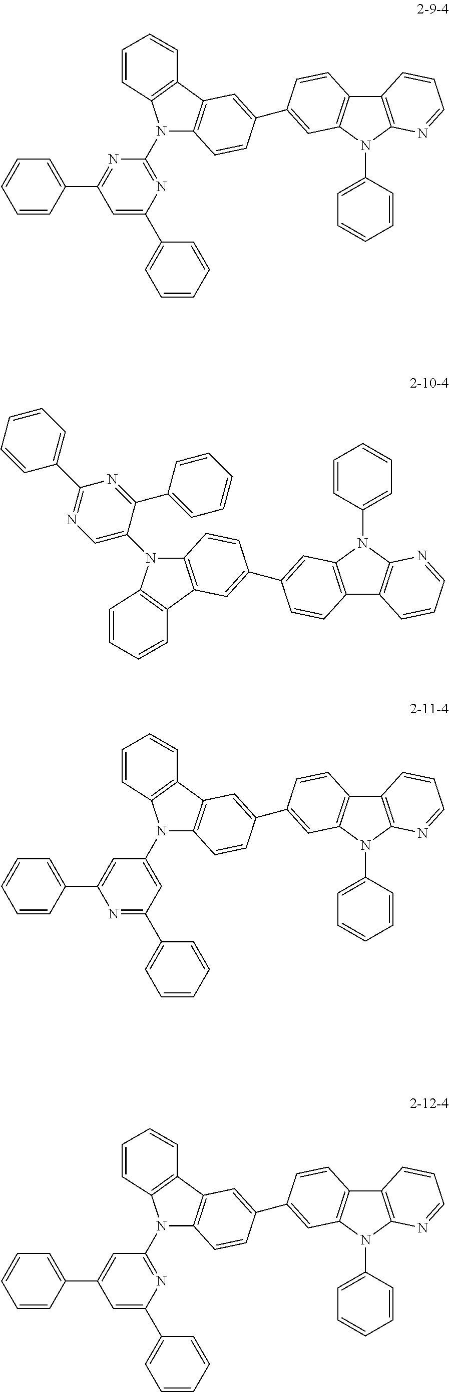 [SMILES: c1ccc(-c2cc(-c3ccccc3)nc(-n3c4ccccc4c4cc(-c5ccc6c7cccnc7n(-c7ccccc7)c6c5)ccc43)n2)cc1 ; c1ccc(-c2ncc(-n3c4ccccc4c4cc(-c5ccc6c7cccnc7n(-c7ccccc7)c6c5)ccc43)c(-c3ccccc3)n2)cc1 ; c1ccc(-c2cc(-n3c4ccccc4c4cc(-c5ccc6c7cccnc7n(-c7ccccc7)c6c5)ccc43)cc(-c3ccccc3)n2)cc1 ; c1ccc(-c2cc(-c3ccccc3)nc(-n3c4ccccc4c4cc(-c5ccc6c7cccnc7n(-c7ccccc7)c6c5)ccc43)c2)cc1]

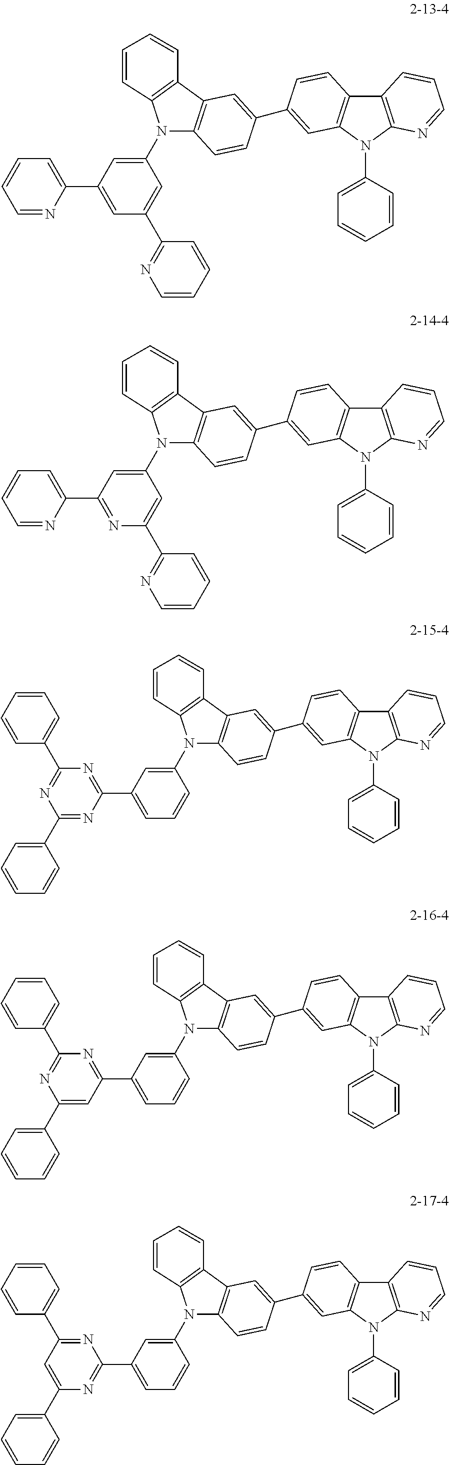 [SMILES: c1ccc(-n2c3cc(-c4ccc5c(c4)c4ccccc4n5-c4cc(-c5ccccn5)cc(-c5ccccn5)c4)ccc3c3cccnc32)cc1 ; c1ccc(-n2c3cc(-c4ccc5c(c4)c4ccccc4n5-c4cc(-c5ccccn5)nc(-c5ccccn5)c4)ccc3c3cccnc32)cc1 ; c1ccc(-c2nc(-c3ccccc3)nc(-c3cccc(-n4c5ccccc5c5cc(-c6ccc7c8cccnc8n(-c8ccccc8)c7c6)ccc54)c3)n2)cc1 ; c1ccc(-c2cc(-c3cccc(-n4c5ccccc5c5cc(-c6ccc7c8cccnc8n(-c8ccccc8)c7c6)ccc54)c3)nc(-c3ccccc3)n2)cc1 ; c1ccc(-c2cc(-c3ccccc3)nc(-c3cccc(-n4c5ccccc5c5cc(-c6ccc7c8cccnc8n(-c8ccccc8)c7c6)ccc54)c3)n2)cc1]

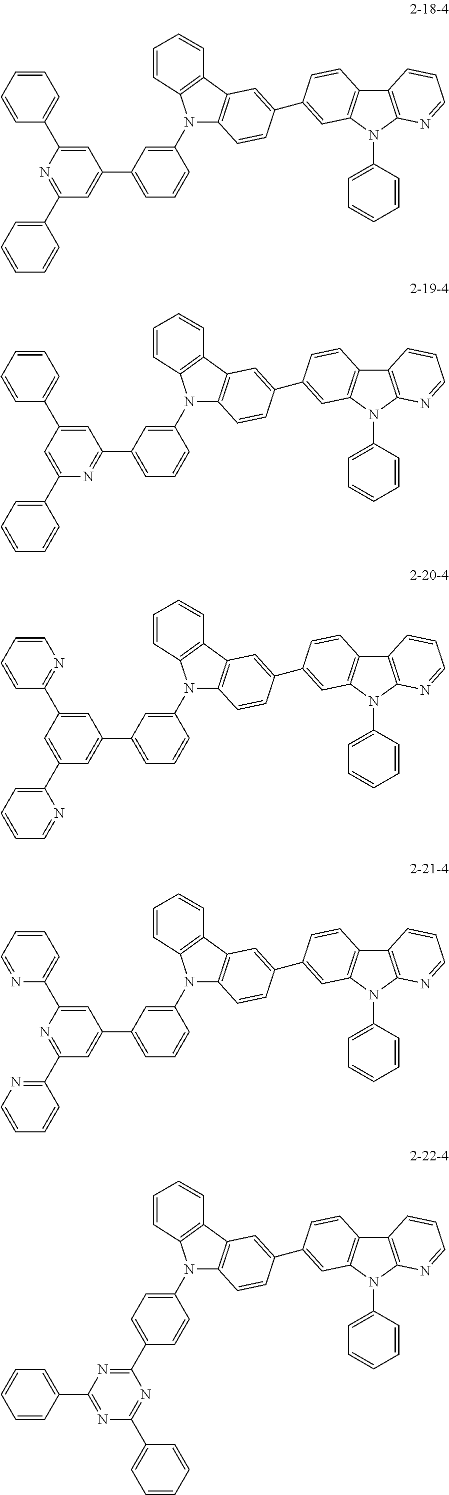 [SMILES: c1ccc(-c2cc(-c3cccc(-n4c5ccccc5c5cc(-c6ccc7c8cccnc8n(-c8ccccc8)c7c6)ccc54)c3)cc(-c3ccccc3)n2)cc1 ; c1ccc(-c2cc(-c3ccccc3)nc(-c3cccc(-n4c5ccccc5c5cc(-c6ccc7c8cccnc8n(-c8ccccc8)c7c6)ccc54)c3)c2)cc1 ; c1ccc(-n2c3cc(-c4ccc5c(c4)c4ccccc4n5-c4cccc(-c5cc(-c6ccccn6)cc(-c6ccccn6)c5)c4)ccc3c3cccnc32)cc1 ; c1ccc(-n2c3cc(-c4ccc5c(c4)c4ccccc4n5-c4cccc(-c5cc(-c6ccccn6)nc(-c6ccccn6)c5)c4)ccc3c3cccnc32)cc1 ; c1ccc(-c2nc(-c3ccccc3)nc(-c3ccc(-n4c5ccccc5c5cc(-c6ccc7c8cccnc8n(-c8ccccc8)c7c6)ccc54)cc3)n2)cc1]

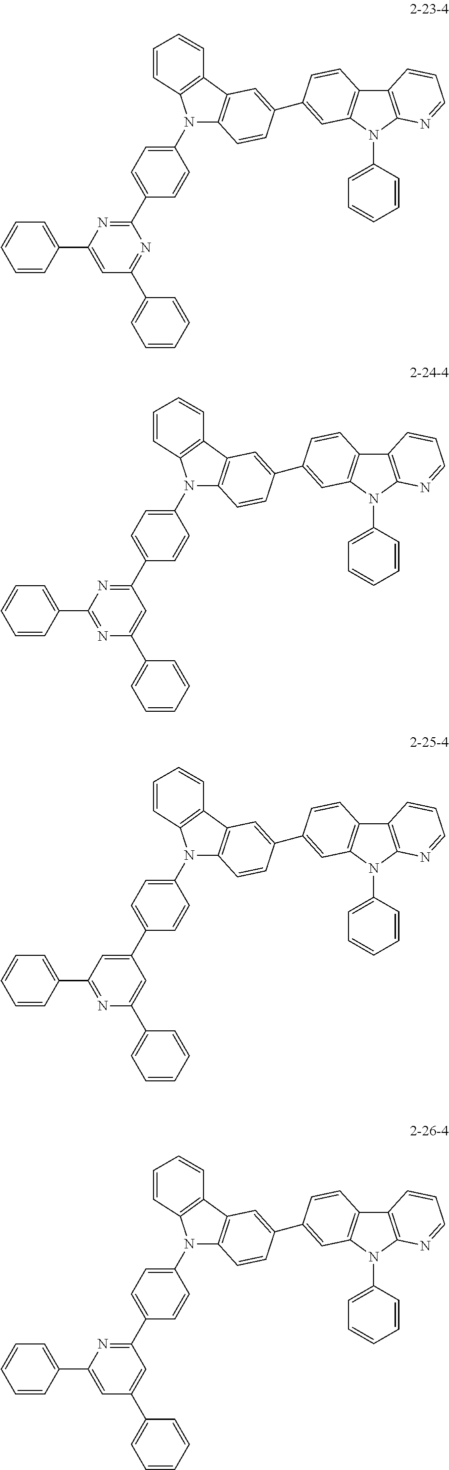 [SMILES: c1ccc(-c2cc(-c3ccccc3)nc(-c3ccc(-n4c5ccccc5c5cc(-c6ccc7c8cccnc8n(-c8ccccc8)c7c6)ccc54)cc3)n2)cc1 ; c1ccc(-c2cc(-c3ccc(-n4c5ccccc5c5cc(-c6ccc7c8cccnc8n(-c8ccccc8)c7c6)ccc54)cc3)nc(-c3ccccc3)n2)cc1 ; c1ccc(-c2cc(-c3ccc(-n4c5ccccc5c5cc(-c6ccc7c8cccnc8n(-c8ccccc8)c7c6)ccc54)cc3)cc(-c3ccccc3)n2)cc1 ; c1ccc(-c2cc(-c3ccccc3)nc(-c3ccc(-n4c5ccccc5c5cc(-c6ccc7c8cccnc8n(-c8ccccc8)c7c6)ccc54)cc3)c2)cc1]

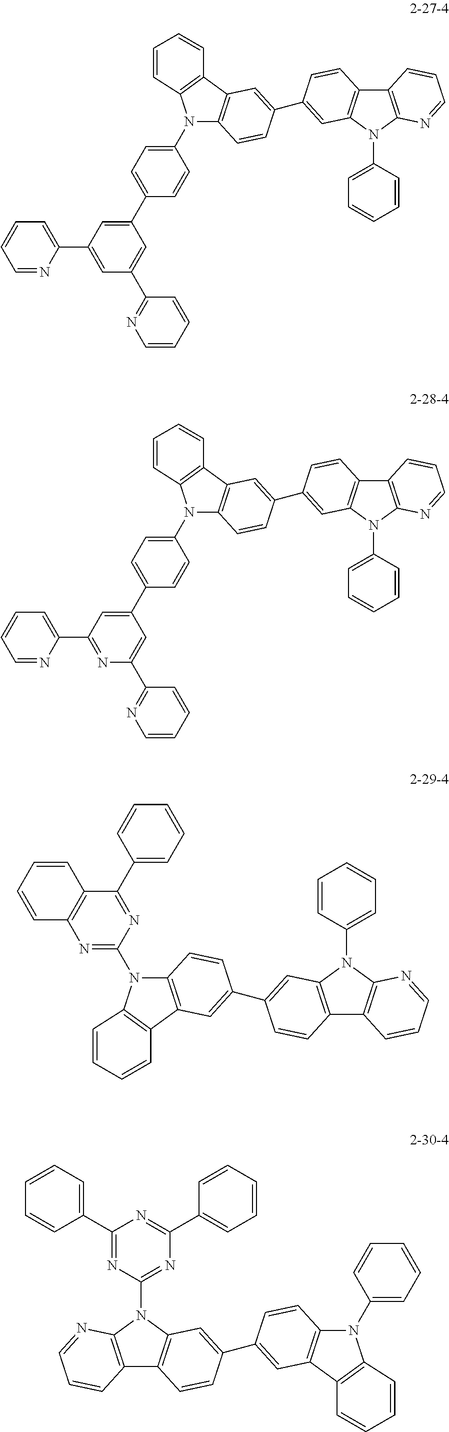 [SMILES: c1ccc(-n2c3cc(-c4ccc5c(c4)c4ccccc4n5-c4ccc(-c5cc(-c6ccccn6)cc(-c6ccccn6)c5)cc4)ccc3c3cccnc32)cc1 ; c1ccc(-n2c3cc(-c4ccc5c(c4)c4ccccc4n5-c4ccc(-c5cc(-c6ccccn6)nc(-c6ccccn6)c5)cc4)ccc3c3cccnc32)cc1 ; c1ccc(-c2nc(-n3c4ccccc4c4cc(-c5ccc6c7cccnc7n(-c7ccccc7)c6c5)ccc43)nc3ccccc23)cc1 ; c1ccc(-c2nc(-c3ccccc3)nc(-n3c4cc(-c5ccc6c(c5)c5ccccc5n6-c5ccccc5)ccc4c4cccnc43)n2)cc1]

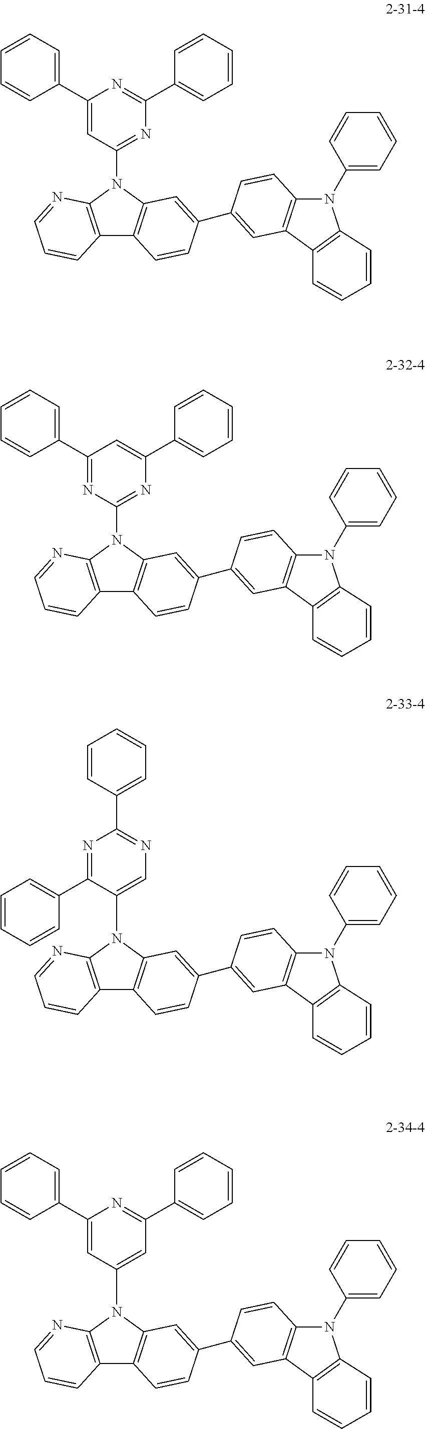 [SMILES: c1ccc(-c2cc(-n3c4cc(-c5ccc6c(c5)c5ccccc5n6-c5ccccc5)ccc4c4cccnc43)nc(-c3ccccc3)n2)cc1 ; c1ccc(-c2cc(-c3ccccc3)nc(-n3c4cc(-c5ccc6c(c5)c5ccccc5n6-c5ccccc5)ccc4c4cccnc43)n2)cc1 ; c1ccc(-c2ncc(-n3c4cc(-c5ccc6c(c5)c5ccccc5n6-c5ccccc5)ccc4c4cccnc43)c(-c3ccccc3)n2)cc1 ; c1ccc(-c2cc(-n3c4cc(-c5ccc6c(c5)c5ccccc5n6-c5ccccc5)ccc4c4cccnc43)cc(-c3ccccc3)n2)cc1]

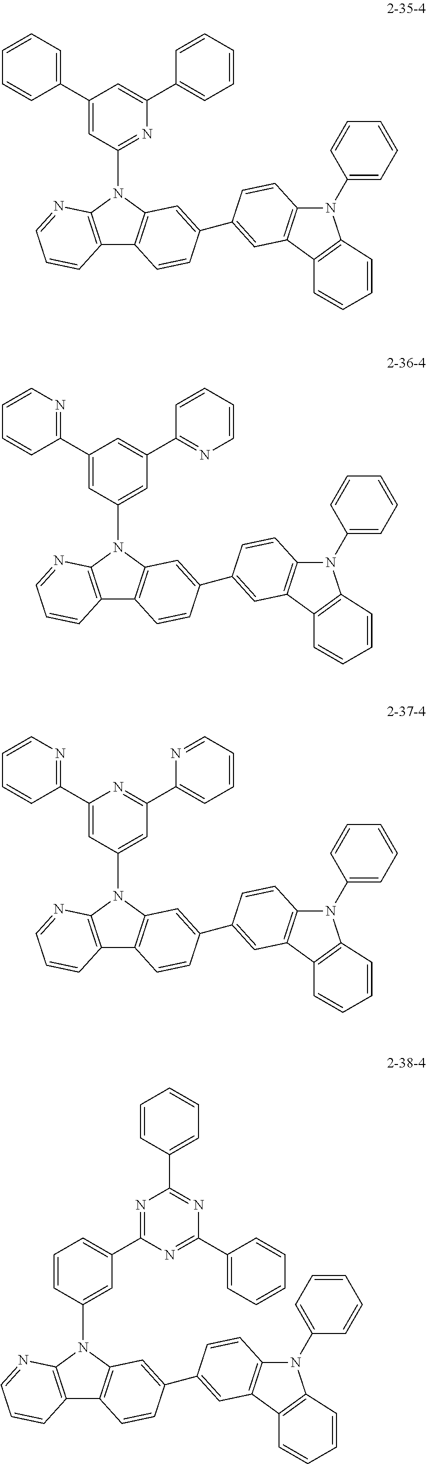 [SMILES: c1ccc(-c2cc(-c3ccccc3)nc(-n3c4cc(-c5ccc6c(c5)c5ccccc5n6-c5ccccc5)ccc4c4cccnc43)c2)cc1 ; c1ccc(-n2c3ccccc3c3cc(-c4ccc5c6cccnc6n(-c6cc(-c7ccccn7)cc(-c7ccccn7)c6)c5c4)ccc32)cc1 ; c1ccc(-n2c3ccccc3c3cc(-c4ccc5c6cccnc6n(-c6cc(-c7ccccn7)nc(-c7ccccn7)c6)c5c4)ccc32)cc1 ; c1ccc(-c2nc(-c3ccccc3)nc(-c3cccc(-n4c5cc(-c6ccc7c(c6)c6ccccc6n7-c6ccccc6)ccc5c5cccnc54)c3)n2)cc1]

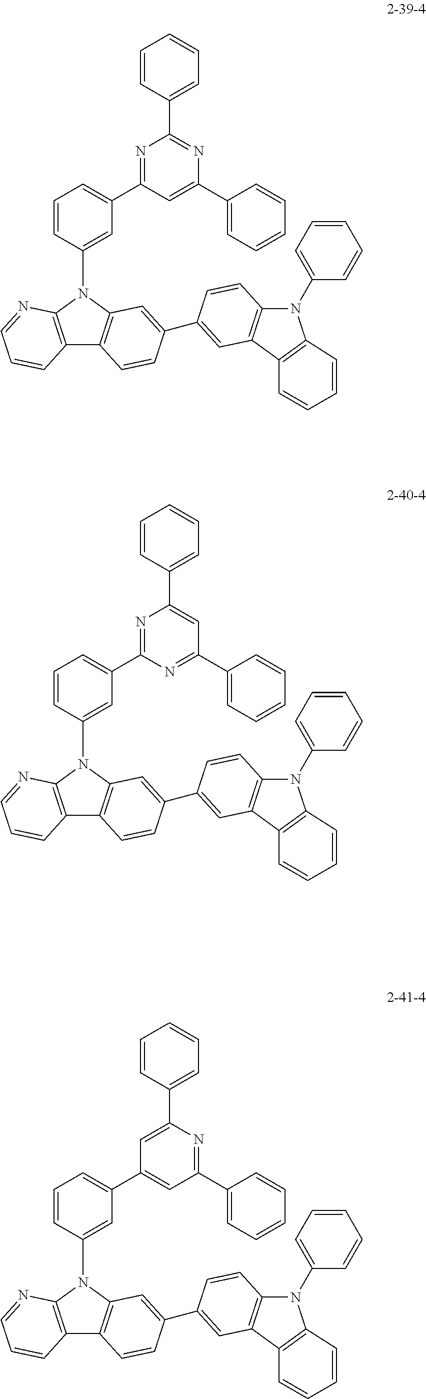 [SMILES: c1ccc(-c2cc(-c3cccc(-n4c5cc(-c6ccc7c(c6)c6ccccc6n7-c6ccccc6)ccc5c5cccnc54)c3)nc(-c3ccccc3)n2)cc1 ; c1ccc(-c2cc(-c3ccccc3)nc(-c3cccc(-n4c5cc(-c6ccc7c(c6)c6ccccc6n7-c6ccccc6)ccc5c5cccnc54)c3)n2)cc1 ; c1ccc(-c2cc(-c3cccc(-n4c5cc(-c6ccc7c(c6)c6ccccc6n7-c6ccccc6)ccc5c5cccnc54)c3)cc(-c3ccccc3)n2)cc1]

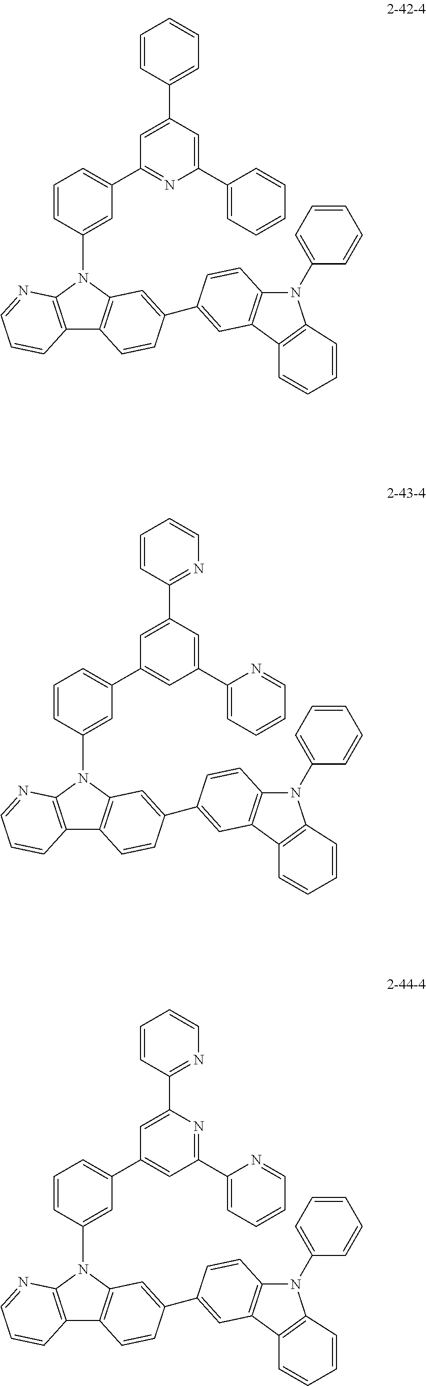 [SMILES: c1ccc(-c2cc(-c3ccccc3)nc(-c3cccc(-n4c5cc(-c6ccc7c(c6)c6ccccc6n7-c6ccccc6)ccc5c5cccnc54)c3)c2)cc1 ; c1ccc(-n2c3ccccc3c3cc(-c4ccc5c6cccnc6n(-c6cccc(-c7cc(-c8ccccn8)cc(-c8ccccn8)c7)c6)c5c4)ccc32)cc1 ; c1ccc(-n2c3ccccc3c3cc(-c4ccc5c6cccnc6n(-c6cccc(-c7cc(-c8ccccn8)nc(-c8ccccn8)c7)c6)c5c4)ccc32)cc1]

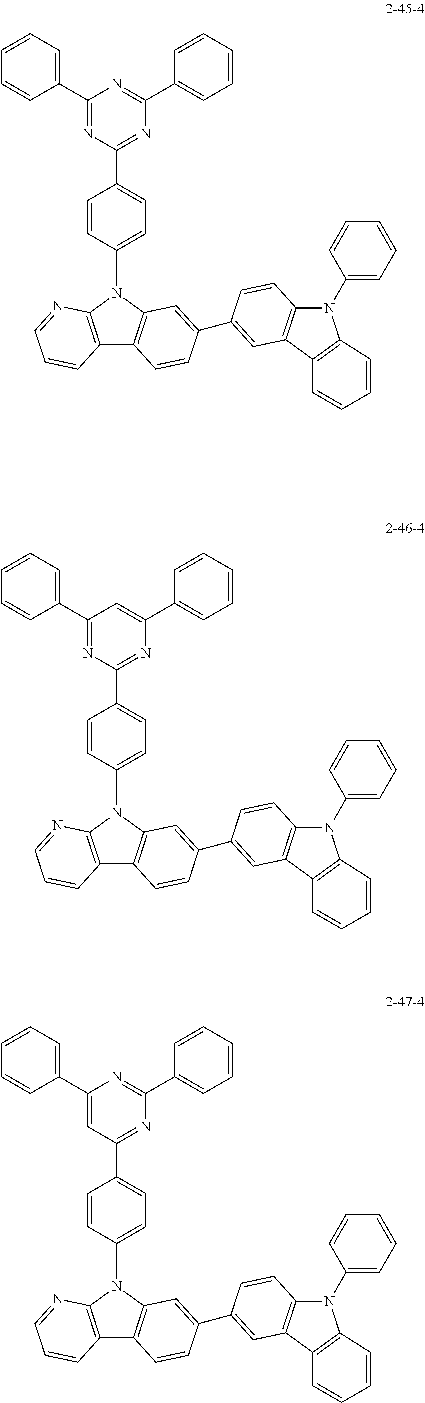 [SMILES: c1ccc(-c2nc(-c3ccccc3)nc(-c3ccc(-n4c5cc(-c6ccc7c(c6)c6ccccc6n7-c6ccccc6)ccc5c5cccnc54)cc3)n2)cc1 ; c1ccc(-c2cc(-c3ccccc3)nc(-c3ccc(-n4c5cc(-c6ccc7c(c6)c6ccccc6n7-c6ccccc6)ccc5c5cccnc54)cc3)n2)cc1 ; c1ccc(-c2cc(-c3ccc(-n4c5cc(-c6ccc7c(c6)c6ccccc6n7-c6ccccc6)ccc5c5cccnc54)cc3)nc(-c3ccccc3)n2)cc1]

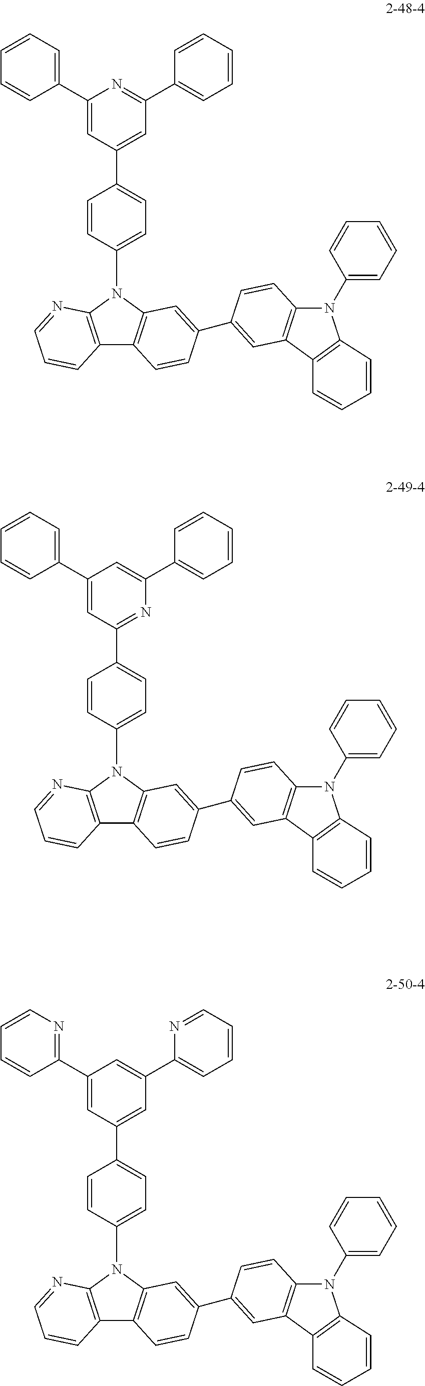 [SMILES: c1ccc(-c2cc(-c3ccc(-n4c5cc(-c6ccc7c(c6)c6ccccc6n7-c6ccccc6)ccc5c5cccnc54)cc3)cc(-c3ccccc3)n2)cc1 ; c1ccc(-c2cc(-c3ccccc3)nc(-c3ccc(-n4c5cc(-c6ccc7c(c6)c6ccccc6n7-c6ccccc6)ccc5c5cccnc54)cc3)c2)cc1 ; c1ccc(-n2c3ccccc3c3cc(-c4ccc5c6cccnc6n(-c6ccc(-c7cc(-c8ccccn8)cc(-c8ccccn8)c7)cc6)c5c4)ccc32)cc1]

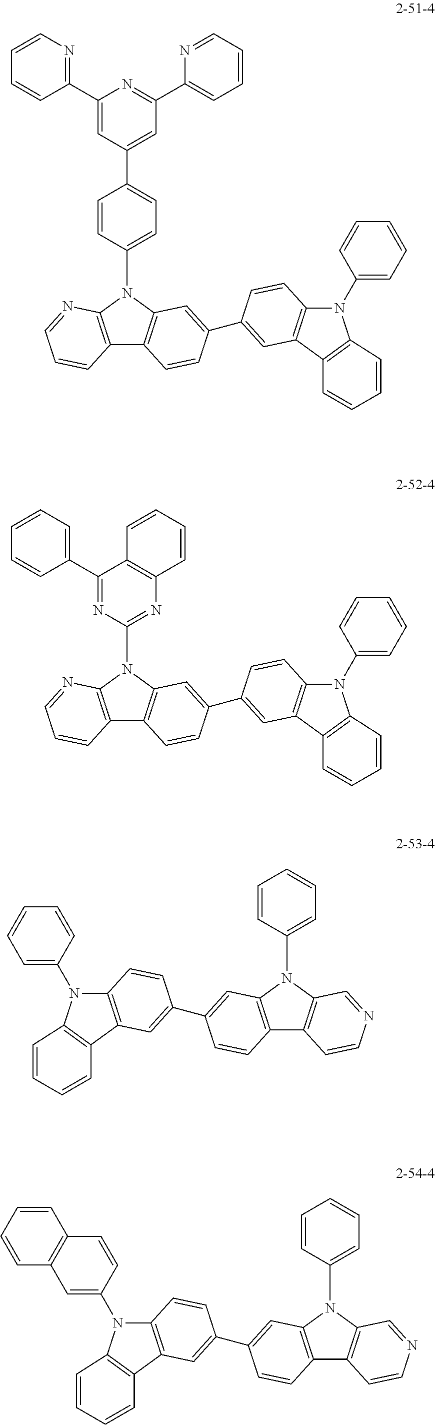 [SMILES: c1ccc(-n2c3ccccc3c3cc(-c4ccc5c6cccnc6n(-c6ccc(-c7cc(-c8ccccn8)nc(-c8ccccn8)c7)cc6)c5c4)ccc32)cc1 ; c1ccc(-c2nc(-n3c4cc(-c5ccc6c(c5)c5ccccc5n6-c5ccccc5)ccc4c4cccnc43)nc3ccccc23)cc1 ; c1ccc(-n2c3ccccc3c3cc(-c4ccc5c6ccncc6n(-c6ccccc6)c5c4)ccc32)cc1 ; c1ccc(-n2c3cnccc3c3ccc(-c4ccc5c(c4)c4ccccc4n5-c4ccc5ccccc5c4)cc32)cc1]

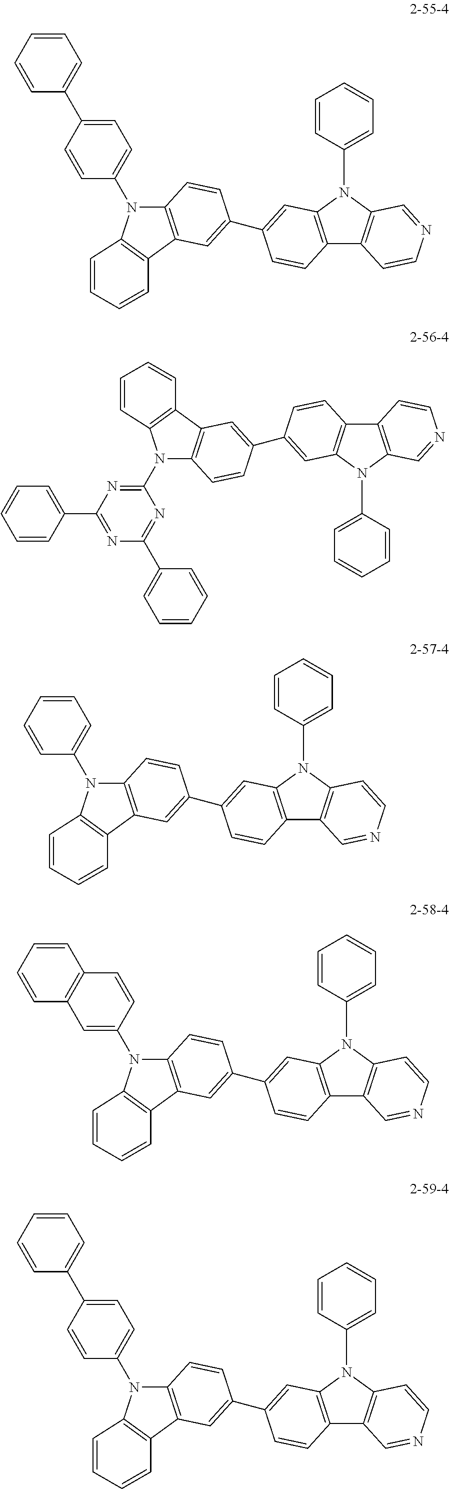 [SMILES: c1ccc(-c2ccc(-n3c4ccccc4c4cc(-c5ccc6c7ccncc7n(-c7ccccc7)c6c5)ccc43)cc2)cc1 ; c1ccc(-c2nc(-c3ccccc3)nc(-n3c4ccccc4c4cc(-c5ccc6c7ccncc7n(-c7ccccc7)c6c5)ccc43)n2)cc1 ; c1ccc(-n2c3ccccc3c3cc(-c4ccc5c6cnccc6n(-c6ccccc6)c5c4)ccc32)cc1 ; c1ccc(-n2c3ccncc3c3ccc(-c4ccc5c(c4)c4ccccc4n5-c4ccc5ccccc5c4)cc32)cc1 ; c1ccc(-c2ccc(-n3c4ccccc4c4cc(-c5ccc6c7cnccc7n(-c7ccccc7)c6c5)ccc43)cc2)cc1]

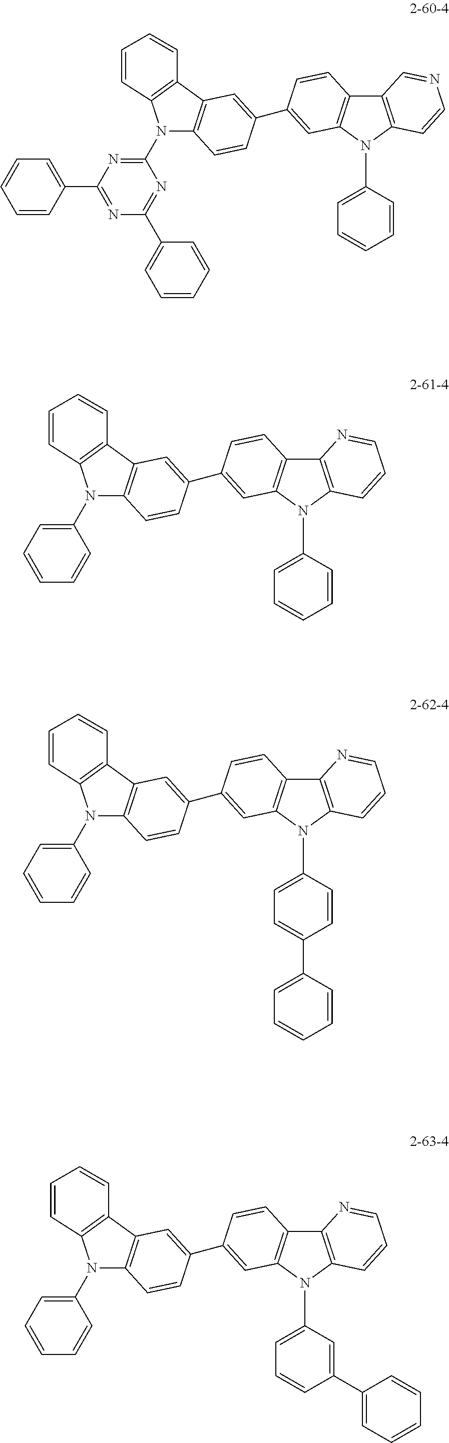 [SMILES: c1ccc(-c2nc(-c3ccccc3)nc(-n3c4ccccc4c4cc(-c5ccc6c7cnccc7n(-c7ccccc7)c6c5)ccc43)n2)cc1 ; c1ccc(-n2c3ccccc3c3cc(-c4ccc5c6ncccc6n(-c6ccccc6)c5c4)ccc32)cc1 ; c1ccc(-c2ccc(-n3c4cc(-c5ccc6c(c5)c5ccccc5n6-c5ccccc5)ccc4c4ncccc43)cc2)cc1 ; c1ccc(-c2cccc(-n3c4cc(-c5ccc6c(c5)c5ccccc5n6-c5ccccc5)ccc4c4ncccc43)c2)cc1]

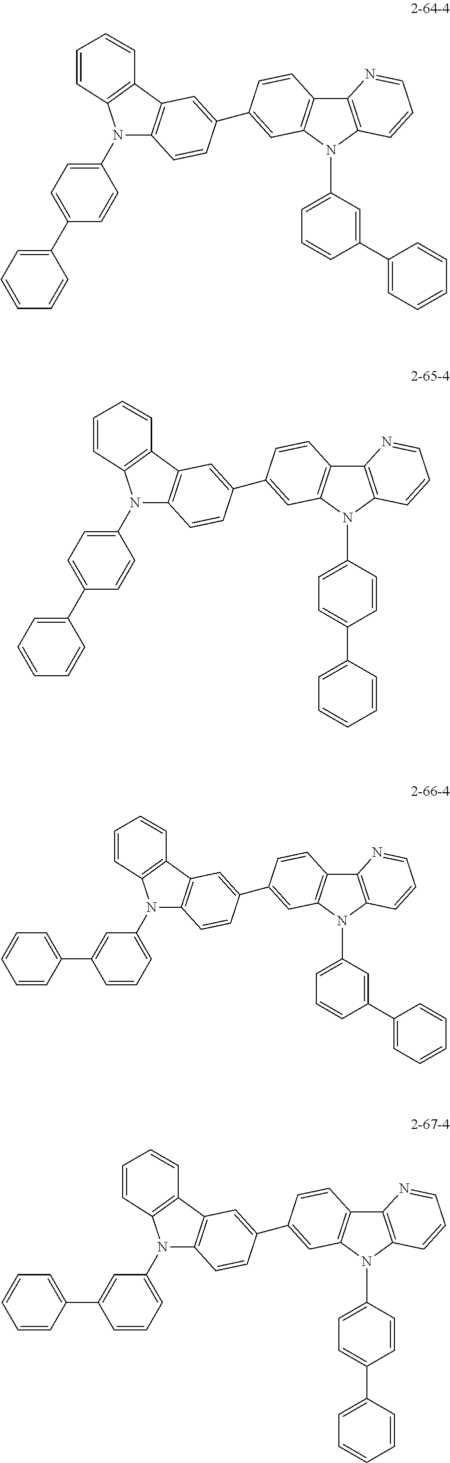 [SMILES: c1ccc(-c2ccc(-n3c4ccccc4c4cc(-c5ccc6c7ncccc7n(-c7cccc(-c8ccccc8)c7)c6c5)ccc43)cc2)cc1 ; c1ccc(-c2ccc(-n3c4ccccc4c4cc(-c5ccc6c7ncccc7n(-c7ccc(-c8ccccc8)cc7)c6c5)ccc43)cc2)cc1 ; c1ccc(-c2cccc(-n3c4ccccc4c4cc(-c5ccc6c7ncccc7n(-c7cccc(-c8ccccc8)c7)c6c5)ccc43)c2)cc1 ; c1ccc(-c2ccc(-n3c4cc(-c5ccc6c(c5)c5ccccc5n6-c5cccc(-c6ccccc6)c5)ccc4c4ncccc43)cc2)cc1]

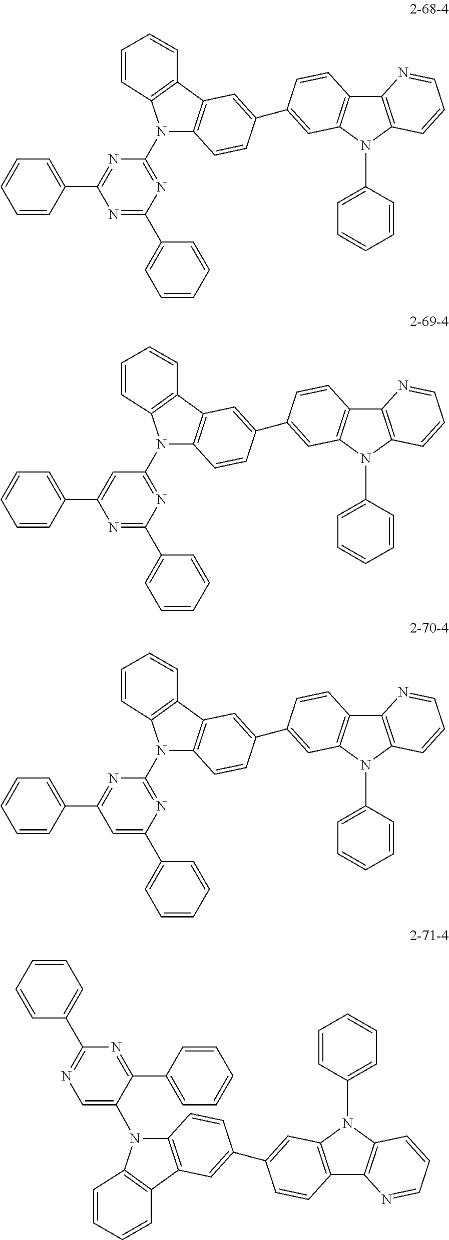

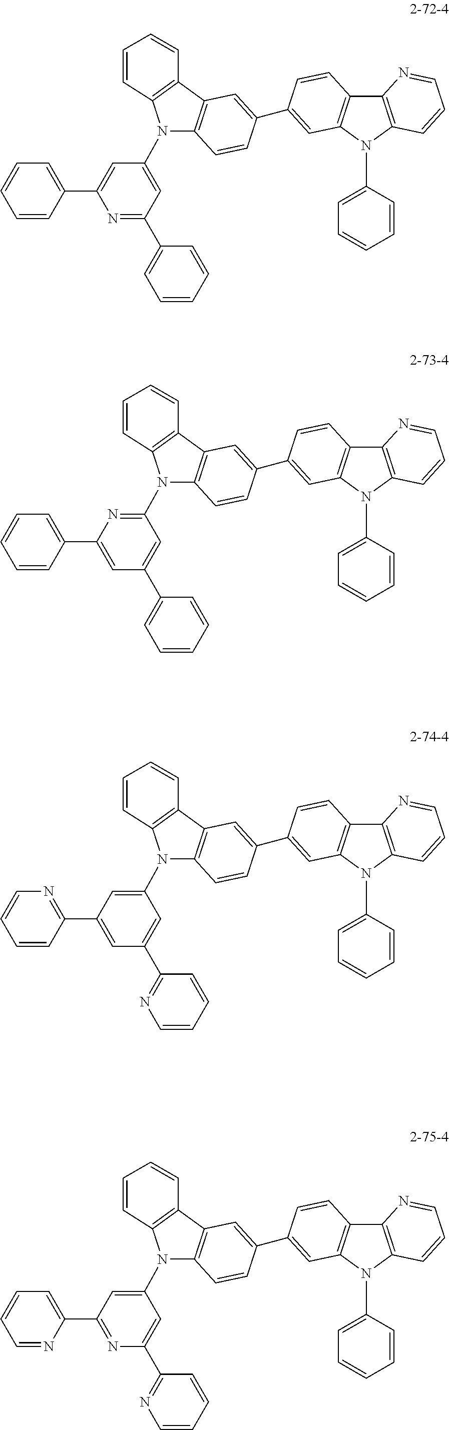 [SMILES: c1ccc(-c2cc(-n3c4ccccc4c4cc(-c5ccc6c7ncccc7n(-c7ccccc7)c6c5)ccc43)cc(-c3ccccc3)n2)cc1 ; c1ccc(-c2cc(-c3ccccc3)nc(-n3c4ccccc4c4cc(-c5ccc6c7ncccc7n(-c7ccccc7)c6c5)ccc43)c2)cc1 ; c1ccc(-n2c3cc(-c4ccc5c(c4)c4ccccc4n5-c4cc(-c5ccccn5)cc(-c5ccccn5)c4)ccc3c3ncccc32)cc1 ; c1ccc(-n2c3cc(-c4ccc5c(c4)c4ccccc4n5-c4cc(-c5ccccn5)nc(-c5ccccn5)c4)ccc3c3ncccc32)cc1]

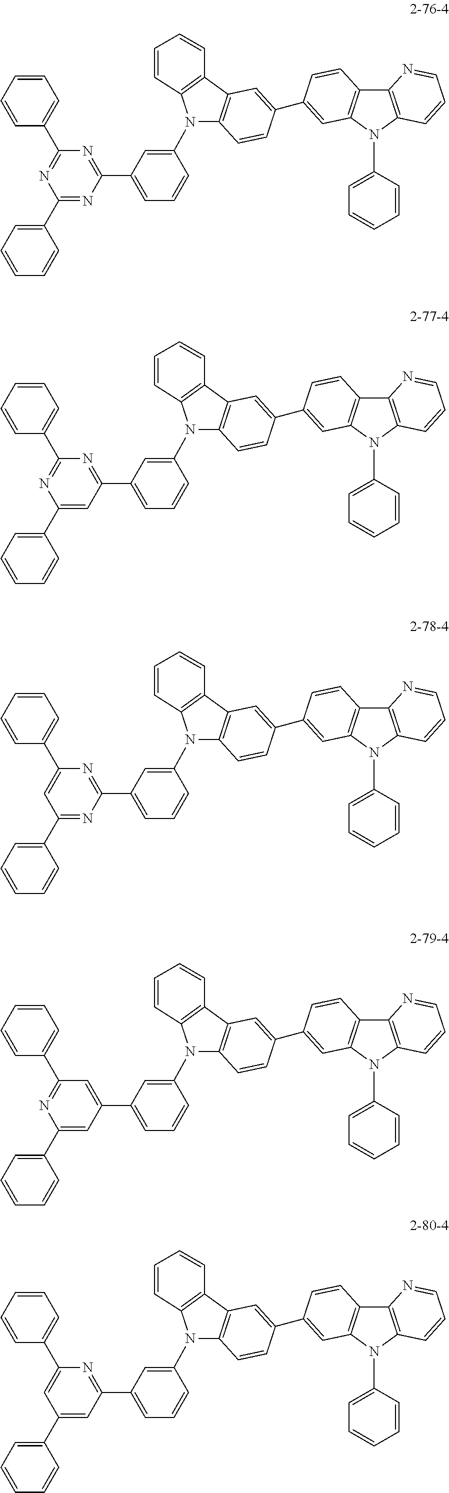 [SMILES: c1ccc(-c2nc(-c3ccccc3)nc(-c3cccc(-n4c5ccccc5c5cc(-c6ccc7c8ncccc8n(-c8ccccc8)c7c6)ccc54)c3)n2)cc1 ; c1ccc(-c2cc(-c3cccc(-n4c5ccccc5c5cc(-c6ccc7c8ncccc8n(-c8ccccc8)c7c6)ccc54)c3)nc(-c3ccccc3)n2)cc1 ; c1ccc(-c2cc(-c3ccccc3)nc(-c3cccc(-n4c5ccccc5c5cc(-c6ccc7c8ncccc8n(-c8ccccc8)c7c6)ccc54)c3)n2)cc1 ; c1ccc(-c2cc(-c3cccc(-n4c5ccccc5c5cc(-c6ccc7c8ncccc8n(-c8ccccc8)c7c6)ccc54)c3)cc(-c3ccccc3)n2)cc1 ; c1ccc(-c2cc(-c3ccccc3)nc(-c3cccc(-n4c5ccccc5c5cc(-c6ccc7c8ncccc8n(-c8ccccc8)c7c6)ccc54)c3)c2)cc1]

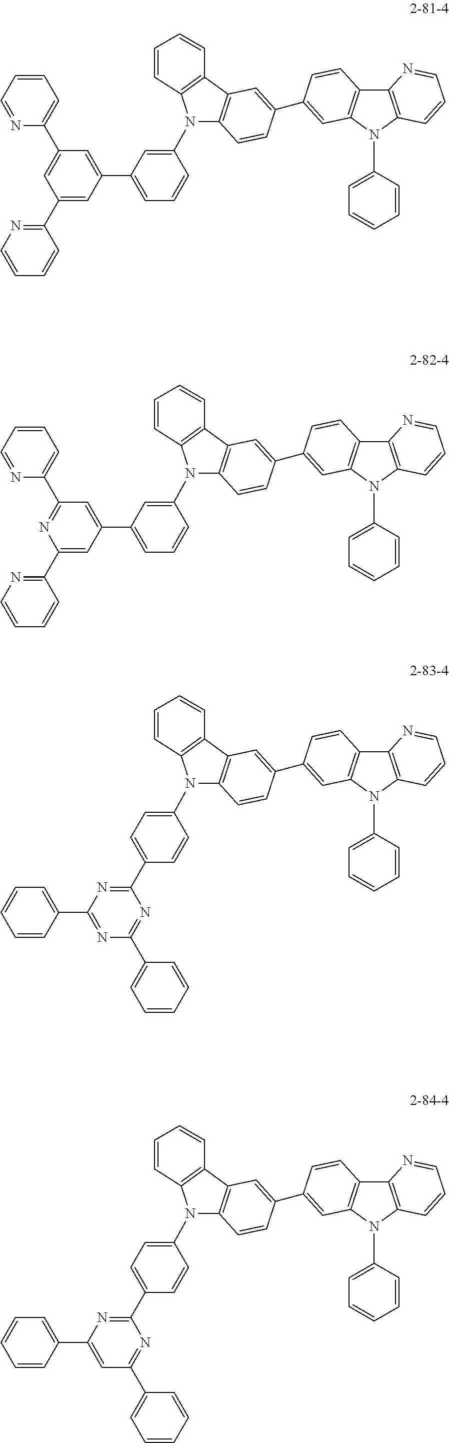 [SMILES: c1ccc(-n2c3cc(-c4ccc5c(c4)c4ccccc4n5-c4cccc(-c5cc(-c6ccccn6)cc(-c6ccccn6)c5)c4)ccc3c3ncccc32)cc1 ; c1ccc(-n2c3cc(-c4ccc5c(c4)c4ccccc4n5-c4cccc(-c5cc(-c6ccccn6)nc(-c6ccccn6)c5)c4)ccc3c3ncccc32)cc1 ; c1ccc(-c2nc(-c3ccccc3)nc(-c3ccc(-n4c5ccccc5c5cc(-c6ccc7c8ncccc8n(-c8ccccc8)c7c6)ccc54)cc3)n2)cc1 ; c1ccc(-c2cc(-c3ccccc3)nc(-c3ccc(-n4c5ccccc5c5cc(-c6ccc7c8ncccc8n(-c8ccccc8)c7c6)ccc54)cc3)n2)cc1]

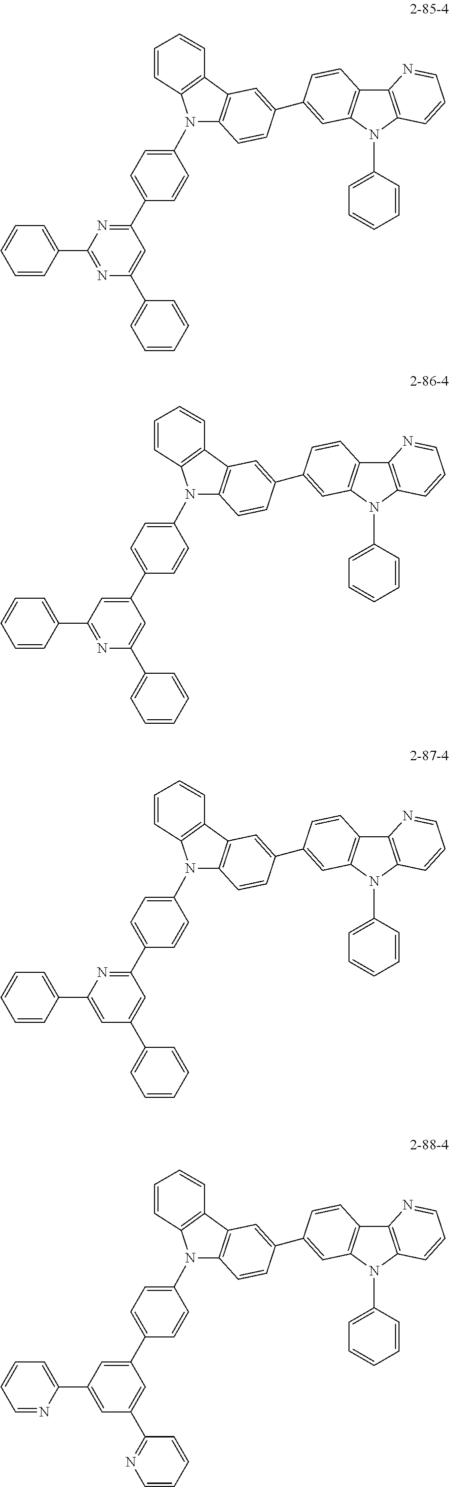 [SMILES: c1ccc(-c2cc(-c3ccc(-n4c5ccccc5c5cc(-c6ccc7c8ncccc8n(-c8ccccc8)c7c6)ccc54)cc3)nc(-c3ccccc3)n2)cc1 ; c1ccc(-c2cc(-c3ccc(-n4c5ccccc5c5cc(-c6ccc7c8ncccc8n(-c8ccccc8)c7c6)ccc54)cc3)cc(-c3ccccc3)n2)cc1 ; c1ccc(-c2cc(-c3ccccc3)nc(-c3ccc(-n4c5ccccc5c5cc(-c6ccc7c8ncccc8n(-c8ccccc8)c7c6)ccc54)cc3)c2)cc1 ; c1ccc(-n2c3cc(-c4ccc5c(c4)c4ccccc4n5-c4ccc(-c5cc(-c6ccccn6)cc(-c6ccccn6)c5)cc4)ccc3c3ncccc32)cc1]

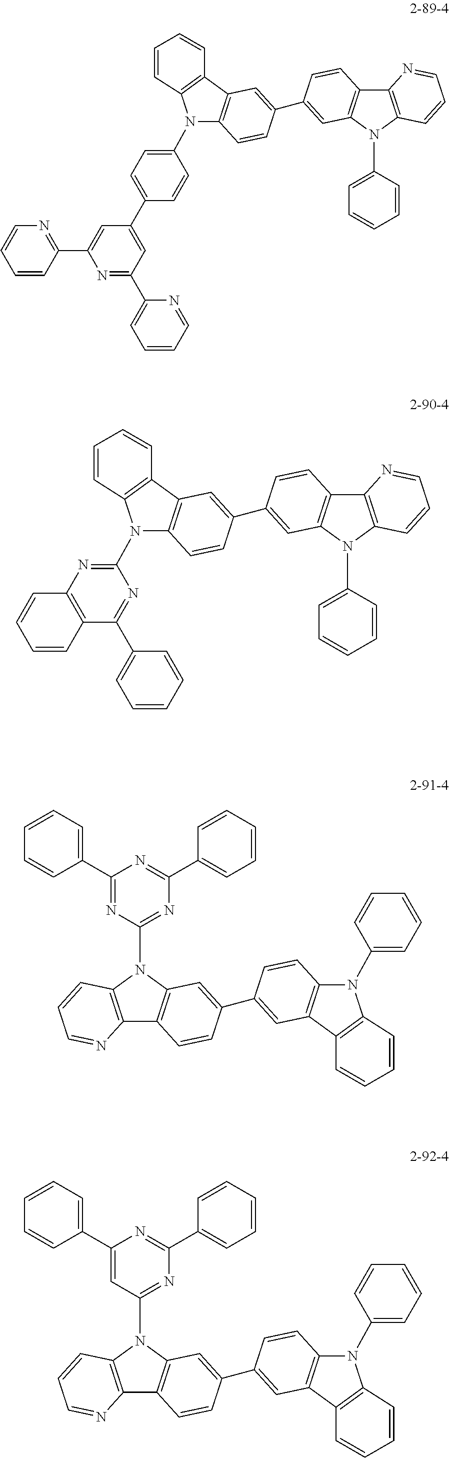 [SMILES: c1ccc(-n2c3cc(-c4ccc5c(c4)c4ccccc4n5-c4ccc(-c5cc(-c6ccccn6)nc(-c6ccccn6)c5)cc4)ccc3c3ncccc32)cc1 ; c1ccc(-c2nc(-n3c4ccccc4c4cc(-c5ccc6c7ncccc7n(-c7ccccc7)c6c5)ccc43)nc3ccccc23)cc1 ; c1ccc(-c2nc(-c3ccccc3)nc(-n3c4cc(-c5ccc6c(c5)c5ccccc5n6-c5ccccc5)ccc4c4ncccc43)n2)cc1 ; c1ccc(-c2cc(-n3c4cc(-c5ccc6c(c5)c5ccccc5n6-c5ccccc5)ccc4c4ncccc43)nc(-c3ccccc3)n2)cc1]

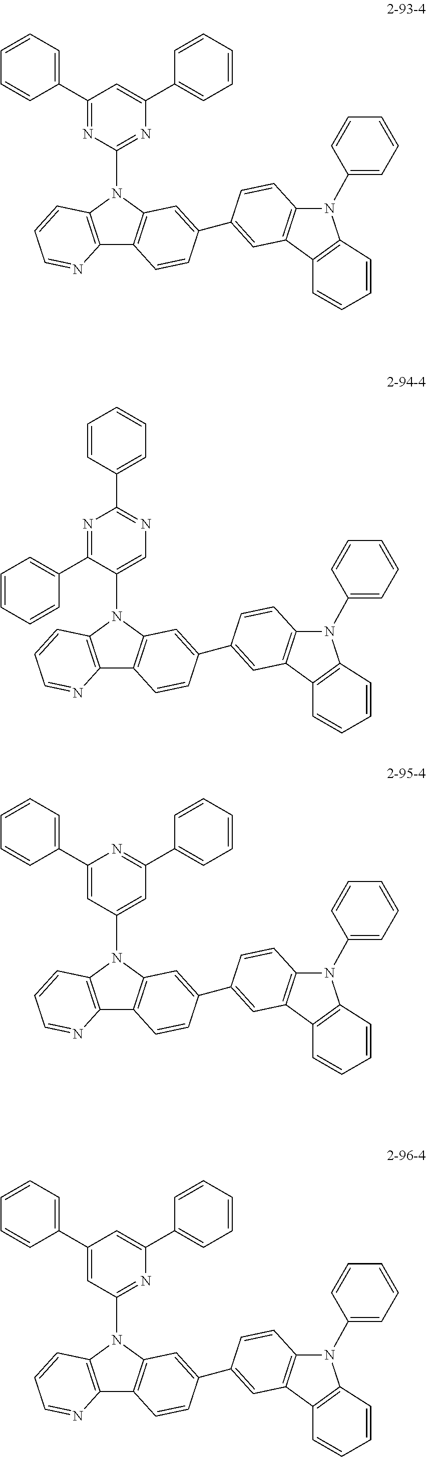 [SMILES: c1ccc(-c2cc(-c3ccccc3)nc(-n3c4cc(-c5ccc6c(c5)c5ccccc5n6-c5ccccc5)ccc4c4ncccc43)n2)cc1 ; c1ccc(-c2ncc(-n3c4cc(-c5ccc6c(c5)c5ccccc5n6-c5ccccc5)ccc4c4ncccc43)c(-c3ccccc3)n2)cc1 ; c1ccc(-c2cc(-n3c4cc(-c5ccc6c(c5)c5ccccc5n6-c5ccccc5)ccc4c4ncccc43)cc(-c3ccccc3)n2)cc1 ; c1ccc(-c2cc(-c3ccccc3)nc(-n3c4cc(-c5ccc6c(c5)c5ccccc5n6-c5ccccc5)ccc4c4ncccc43)c2)cc1]

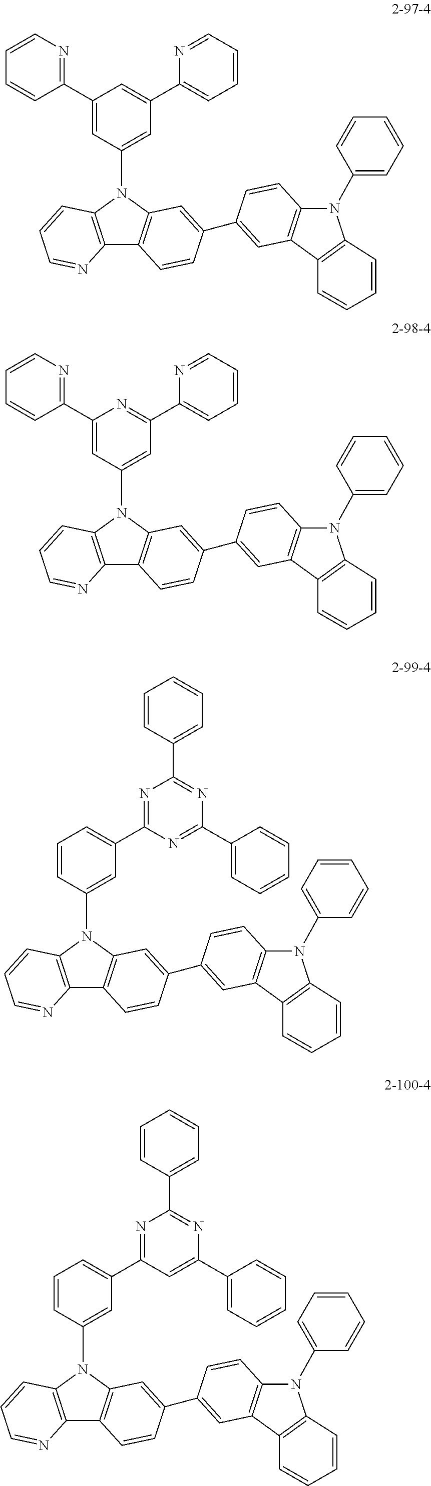 [SMILES: c1ccc(-n2c3ccccc3c3cc(-c4ccc5c6ncccc6n(-c6cc(-c7ccccn7)cc(-c7ccccn7)c6)c5c4)ccc32)cc1 ; c1ccc(-n2c3ccccc3c3cc(-c4ccc5c6ncccc6n(-c6cc(-c7ccccn7)nc(-c7ccccn7)c6)c5c4)ccc32)cc1 ; c1ccc(-c2nc(-c3ccccc3)nc(-c3cccc(-n4c5cc(-c6ccc7c(c6)c6ccccc6n7-c6ccccc6)ccc5c5ncccc54)c3)n2)cc1 ; c1ccc(-c2cc(-c3cccc(-n4c5cc(-c6ccc7c(c6)c6ccccc6n7-c6ccccc6)ccc5c5ncccc54)c3)nc(-c3ccccc3)n2)cc1]

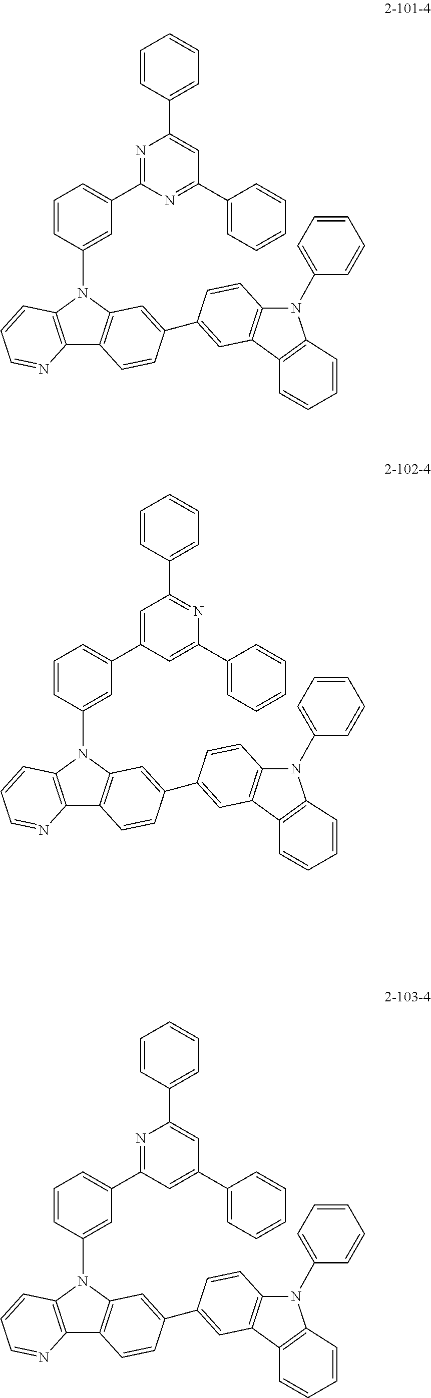 [SMILES: c1ccc(-c2cc(-c3ccccc3)nc(-c3cccc(-n4c5cc(-c6ccc7c(c6)c6ccccc6n7-c6ccccc6)ccc5c5ncccc54)c3)n2)cc1 ; c1ccc(-c2cc(-c3cccc(-n4c5cc(-c6ccc7c(c6)c6ccccc6n7-c6ccccc6)ccc5c5ncccc54)c3)cc(-c3ccccc3)n2)cc1 ; c1ccc(-c2cc(-c3ccccc3)nc(-c3cccc(-n4c5cc(-c6ccc7c(c6)c6ccccc6n7-c6ccccc6)ccc5c5ncccc54)c3)c2)cc1]

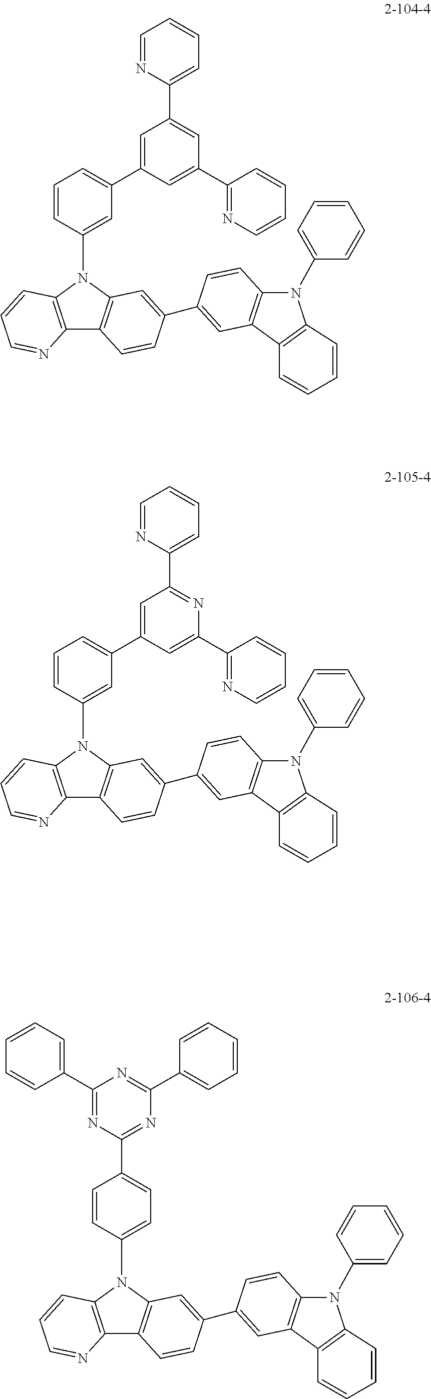 [SMILES: c1ccc(-n2c3ccccc3c3cc(-c4ccc5c6ncccc6n(-c6cccc(-c7cc(-c8ccccn8)cc(-c8ccccn8)c7)c6)c5c4)ccc32)cc1 ; c1ccc(-n2c3ccccc3c3cc(-c4ccc5c6ncccc6n(-c6cccc(-c7cc(-c8ccccn8)nc(-c8ccccn8)c7)c6)c5c4)ccc32)cc1 ; c1ccc(-c2nc(-c3ccccc3)nc(-c3ccc(-n4c5cc(-c6ccc7c(c6)c6ccccc6n7-c6ccccc6)ccc5c5ncccc54)cc3)n2)cc1]

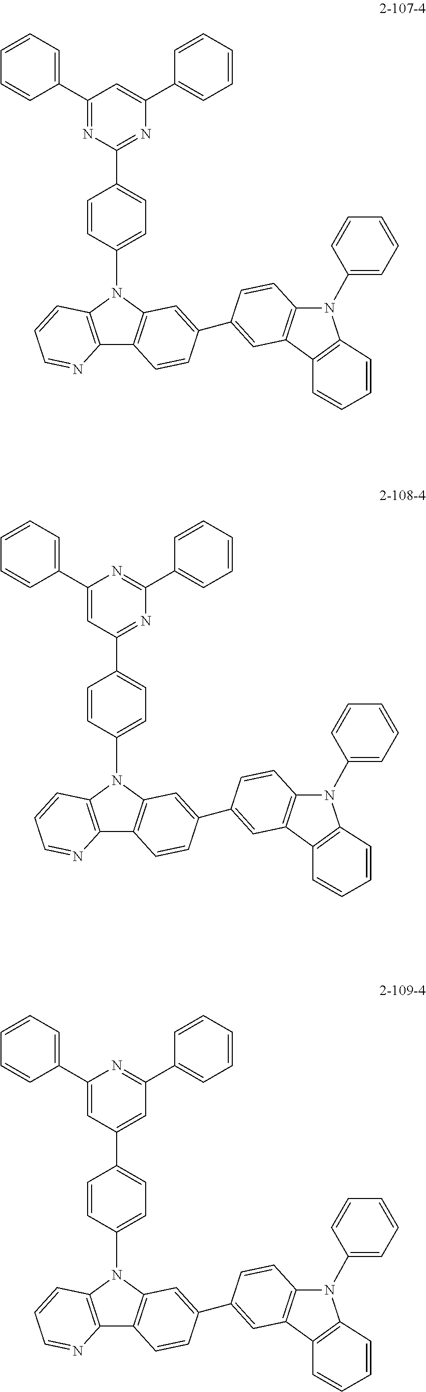 [SMILES: c1ccc(-c2cc(-c3ccccc3)nc(-c3ccc(-n4c5cc(-c6ccc7c(c6)c6ccccc6n7-c6ccccc6)ccc5c5ncccc54)cc3)n2)cc1 ; c1ccc(-c2cc(-c3ccc(-n4c5cc(-c6ccc7c(c6)c6ccccc6n7-c6ccccc6)ccc5c5ncccc54)cc3)nc(-c3ccccc3)n2)cc1 ; c1ccc(-c2cc(-c3ccc(-n4c5cc(-c6ccc7c(c6)c6ccccc6n7-c6ccccc6)ccc5c5ncccc54)cc3)cc(-c3ccccc3)n2)cc1]

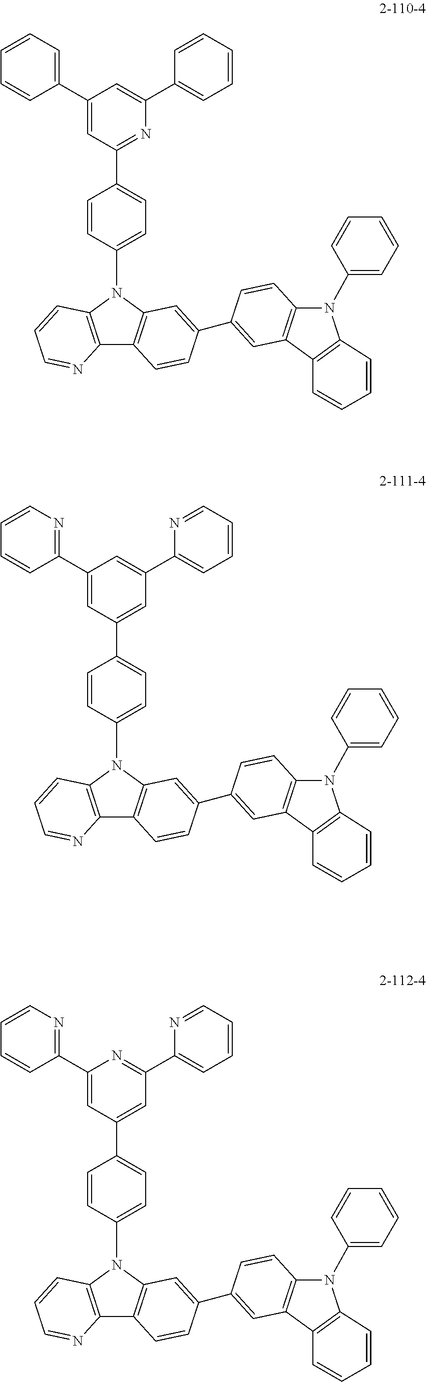 [SMILES: c1ccc(-c2cc(-c3ccccc3)nc(-c3ccc(-n4c5cc(-c6ccc7c(c6)c6ccccc6n7-c6ccccc6)ccc5c5ncccc54)cc3)c2)cc1 ; c1ccc(-n2c3ccccc3c3cc(-c4ccc5c6ncccc6n(-c6ccc(-c7cc(-c8ccccn8)cc(-c8ccccn8)c7)cc6)c5c4)ccc32)cc1 ; c1ccc(-n2c3ccccc3c3cc(-c4ccc5c6ncccc6n(-c6ccc(-c7cc(-c8ccccn8)nc(-c8ccccn8)c7)cc6)c5c4)ccc32)cc1]

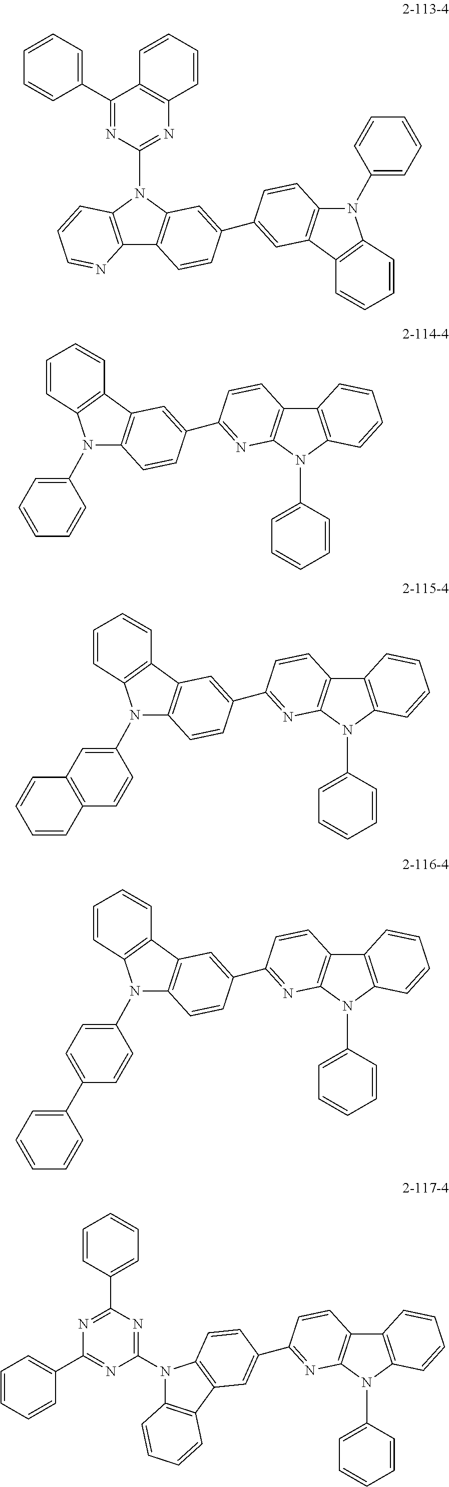 [SMILES: c1ccc(-c2nc(-n3c4cc(-c5ccc6c(c5)c5ccccc5n6-c5ccccc5)ccc4c4ncccc43)nc3ccccc23)cc1 ; c1ccc(-n2c3ccccc3c3cc(-c4ccc5c6ccccc6n(-c6ccccc6)c5n4)ccc32)cc1 ; c1ccc(-n2c3ccccc3c3ccc(-c4ccc5c(c4)c4ccccc4n5-c4ccc5ccccc5c4)nc32)cc1 ; c1ccc(-c2ccc(-n3c4ccccc4c4cc(-c5ccc6c7ccccc7n(-c7ccccc7)c6n5)ccc43)cc2)cc1 ; c1ccc(-c2nc(-c3ccccc3)nc(-n3c4ccccc4c4cc(-c5ccc6c7ccccc7n(-c7ccccc7)c6n5)ccc43)n2)cc1]

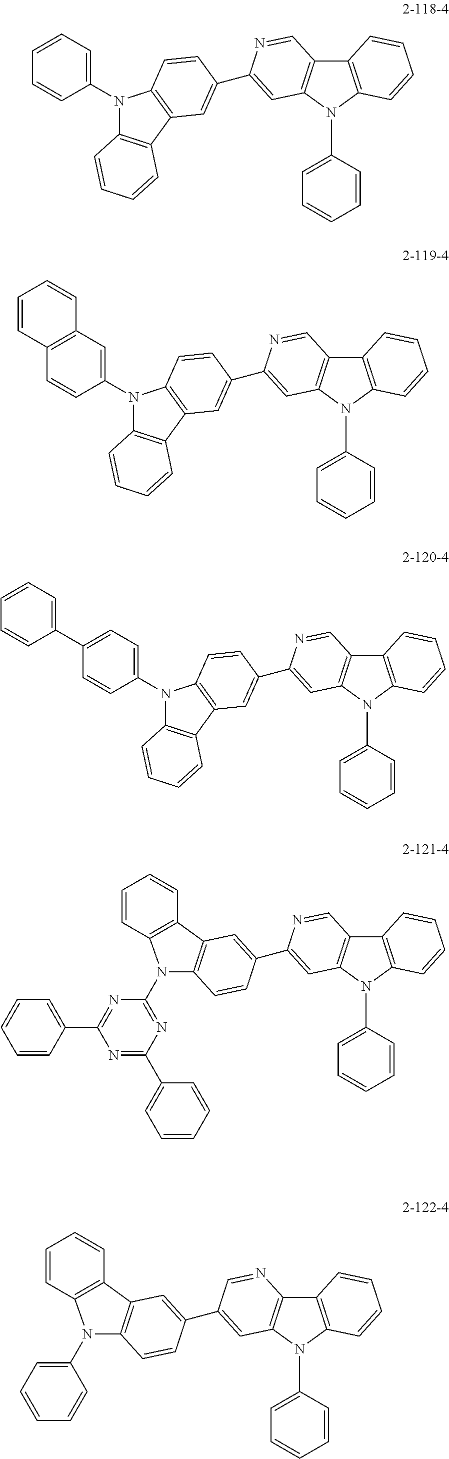 [SMILES: c1ccc(-n2c3ccccc3c3cc(-c4cc5c(cn4)c4ccccc4n5-c4ccccc4)ccc32)cc1 ; c1ccc(-n2c3ccccc3c3cnc(-c4ccc5c(c4)c4ccccc4n5-c4ccc5ccccc5c4)cc32)cc1 ; c1ccc(-c2ccc(-n3c4ccccc4c4cc(-c5cc6c(cn5)c5ccccc5n6-c5ccccc5)ccc43)cc2)cc1 ; c1ccc(-c2nc(-c3ccccc3)nc(-n3c4ccccc4c4cc(-c5cc6c(cn5)c5ccccc5n6-c5ccccc5)ccc43)n2)cc1 ; c1ccc(-n2c3ccccc3c3cc(-c4cnc5c6ccccc6n(-c6ccccc6)c5c4)ccc32)cc1]

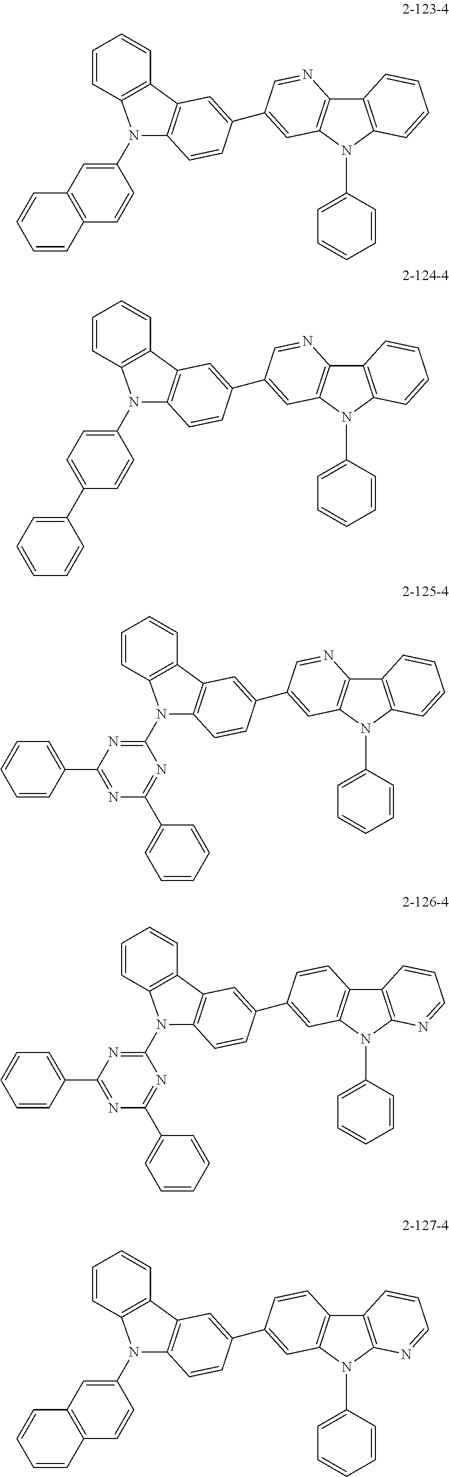 [SMILES: c1ccc(-n2c3ccccc3c3ncc(-c4ccc5c(c4)c4ccccc4n5-c4ccc5ccccc5c4)cc32)cc1 ; c1ccc(-c2ccc(-n3c4ccccc4c4cc(-c5cnc6c7ccccc7n(-c7ccccc7)c6c5)ccc43)cc2)cc1 ; c1ccc(-c2nc(-c3ccccc3)nc(-n3c4ccccc4c4cc(-c5cnc6c7ccccc7n(-c7ccccc7)c6c5)ccc43)n2)cc1 ; c1ccc(-c2nc(-c3ccccc3)nc(-n3c4ccccc4c4cc(-c5ccc6c7cccnc7n(-c7ccccc7)c6c5)ccc43)n2)cc1 ; c1ccc(-n2c3cc(-c4ccc5c(c4)c4ccccc4n5-c4ccc5ccccc5c4)ccc3c3cccnc32)cc1]

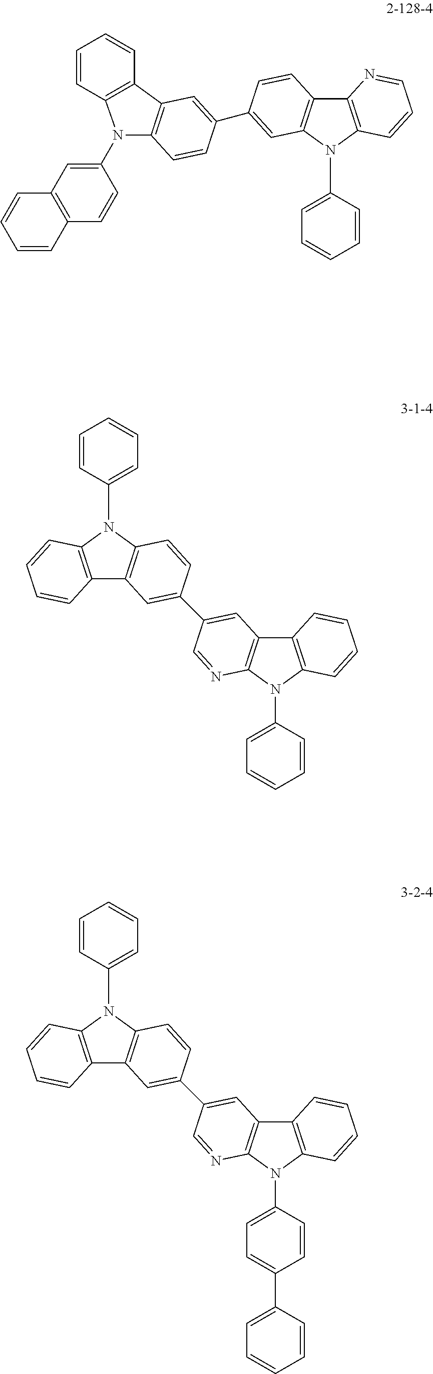 [SMILES: c1ccc(-n2c3cc(-c4ccc5c(c4)c4ccccc4n5-c4ccc5ccccc5c4)ccc3c3ncccc32)cc1 ; c1ccc(-n2c3ccccc3c3cc(-c4cnc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccccc5)cnc43)cc2)cc1]

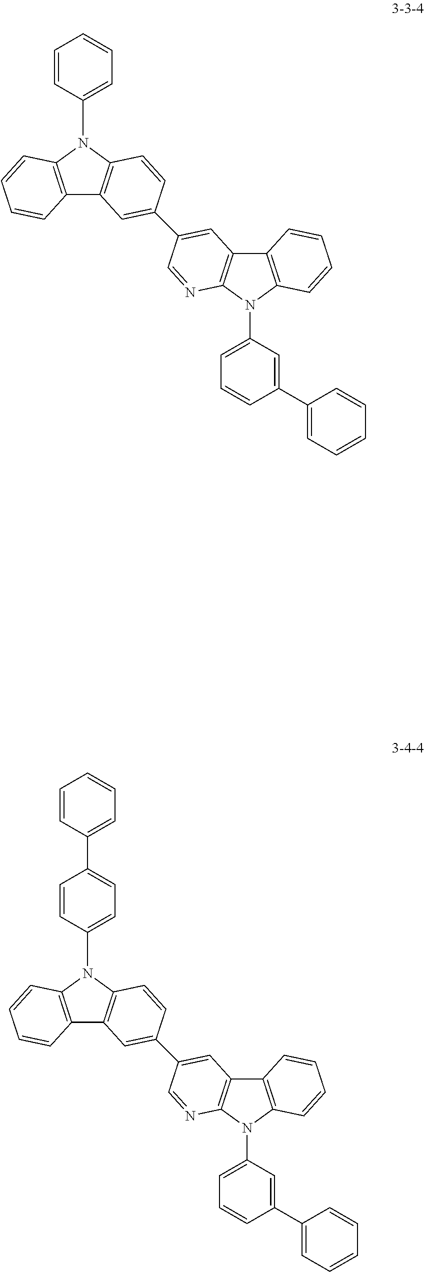 [SMILES: c1ccc(-c2cccc(-n3c4ccccc4c4cc(-c5ccc6c(c5)c5ccccc5n6-c5ccccc5)cnc43)c2)cc1 ; c1ccc(-c2ccc(-n3c4ccccc4c4cc(-c5cnc6c(c5)c5ccccc5n6-c5cccc(-c6ccccc6)c5)ccc43)cc2)cc1]

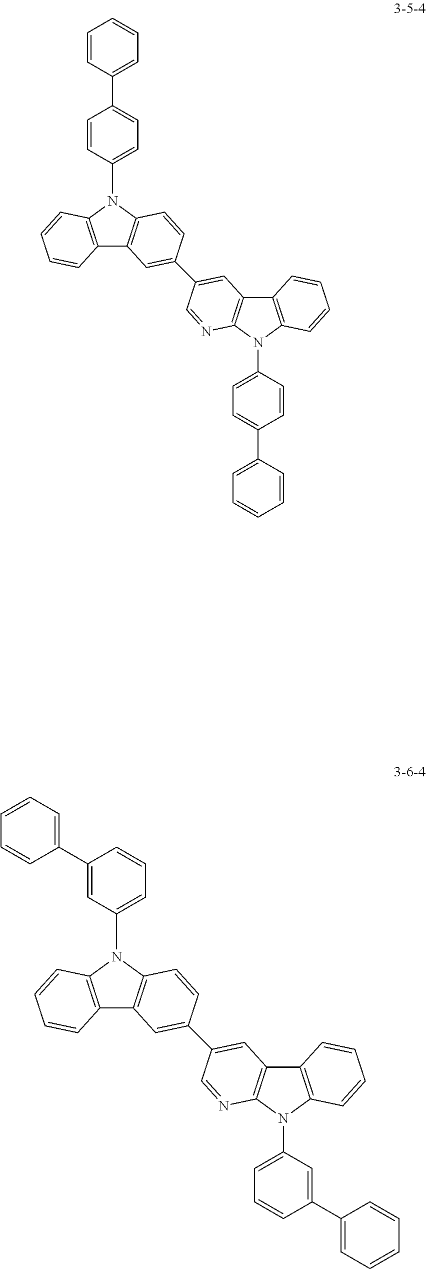 [SMILES: c1ccc(-c2ccc(-n3c4ccccc4c4cc(-c5cnc6c(c5)c5ccccc5n6-c5ccc(-c6ccccc6)cc5)ccc43)cc2)cc1 ; c1ccc(-c2cccc(-n3c4ccccc4c4cc(-c5cnc6c(c5)c5ccccc5n6-c5cccc(-c6ccccc6)c5)ccc43)c2)cc1]

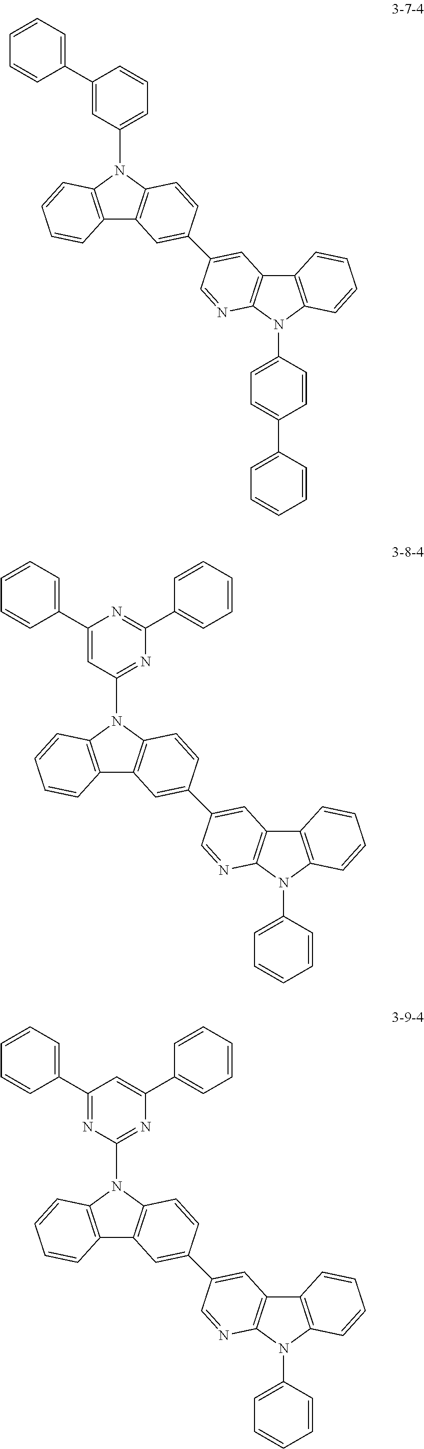 [SMILES: c1ccc(-c2ccc(-n3c4ccccc4c4cc(-c5ccc6c(c5)c5ccccc5n6-c5cccc(-c6ccccc6)c5)cnc43)cc2)cc1 ; c1ccc(-c2cc(-n3c4ccccc4c4cc(-c5cnc6c(c5)c5ccccc5n6-c5ccccc5)ccc43)nc(-c3ccccc3)n2)cc1 ; c1ccc(-c2cc(-c3ccccc3)nc(-n3c4ccccc4c4cc(-c5cnc6c(c5)c5ccccc5n6-c5ccccc5)ccc43)n2)cc1]

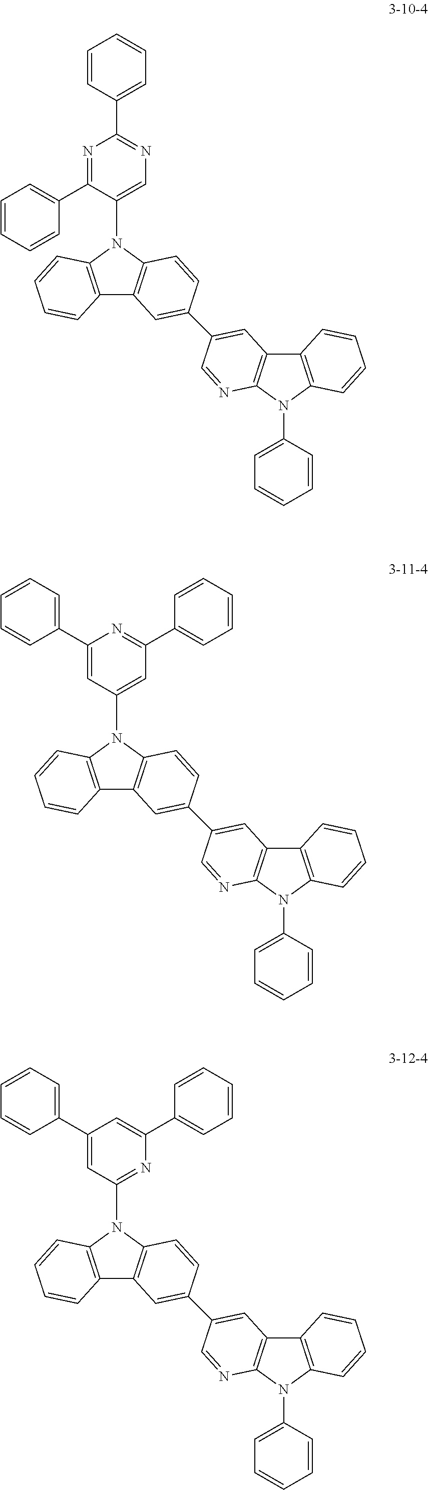 [SMILES: c1ccc(-c2ncc(-n3c4ccccc4c4cc(-c5cnc6c(c5)c5ccccc5n6-c5ccccc5)ccc43)c(-c3ccccc3)n2)cc1 ; c1ccc(-c2cc(-n3c4ccccc4c4cc(-c5cnc6c(c5)c5ccccc5n6-c5ccccc5)ccc43)cc(-c3ccccc3)n2)cc1 ; c1ccc(-c2cc(-c3ccccc3)nc(-n3c4ccccc4c4cc(-c5cnc6c(c5)c5ccccc5n6-c5ccccc5)ccc43)c2)cc1]

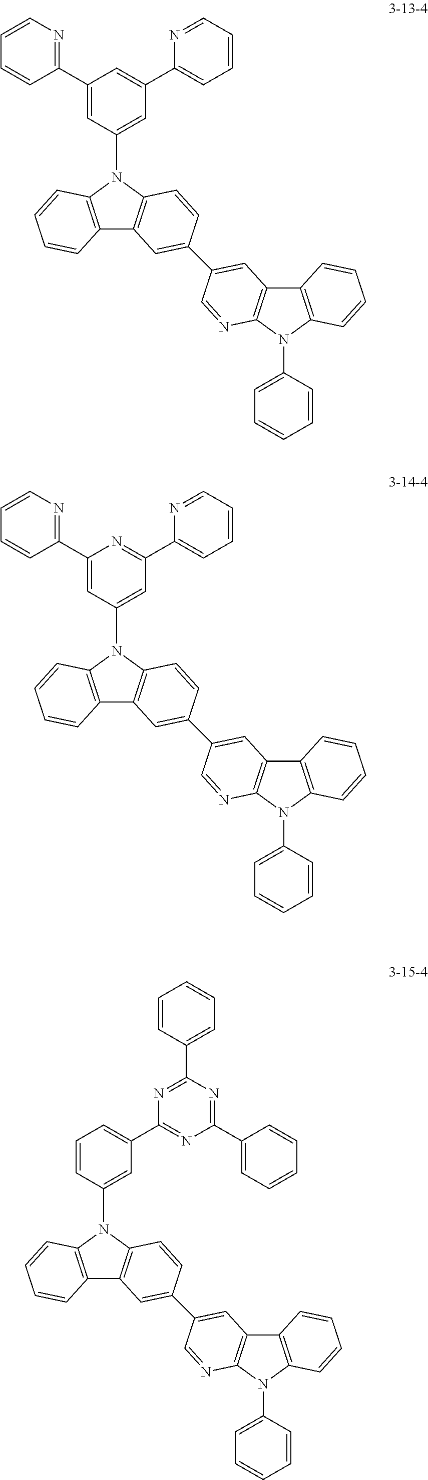 [SMILES: c1ccc(-n2c3ccccc3c3cc(-c4ccc5c(c4)c4ccccc4n5-c4cc(-c5ccccn5)cc(-c5ccccn5)c4)cnc32)cc1 ; c1ccc(-n2c3ccccc3c3cc(-c4ccc5c(c4)c4ccccc4n5-c4cc(-c5ccccn5)nc(-c5ccccn5)c4)cnc32)cc1 ; c1ccc(-c2nc(-c3ccccc3)nc(-c3cccc(-n4c5ccccc5c5cc(-c6cnc7c(c6)c6ccccc6n7-c6ccccc6)ccc54)c3)n2)cc1]

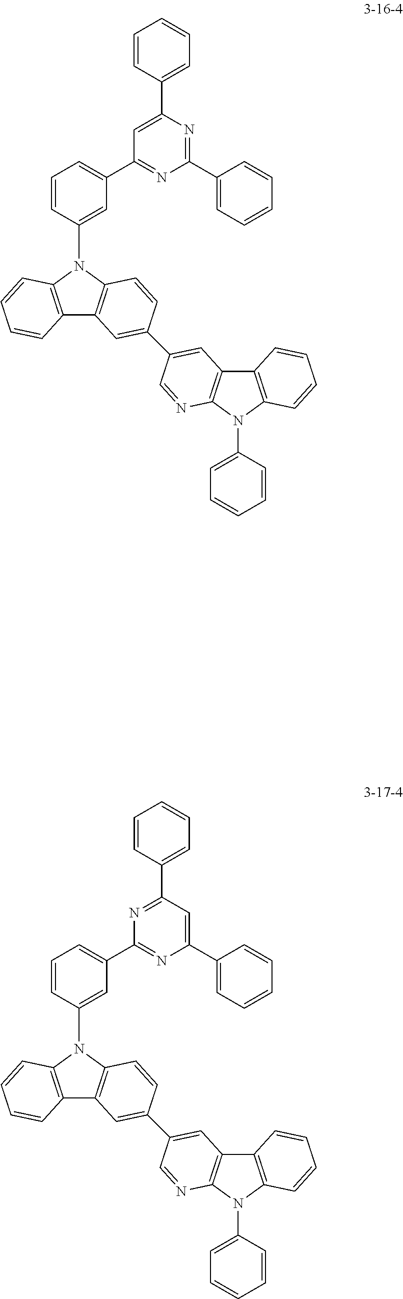 [SMILES: c1ccc(-c2cc(-c3cccc(-n4c5ccccc5c5cc(-c6cnc7c(c6)c6ccccc6n7-c6ccccc6)ccc54)c3)nc(-c3ccccc3)n2)cc1 ; c1ccc(-c2cc(-c3ccccc3)nc(-c3cccc(-n4c5ccccc5c5cc(-c6cnc7c(c6)c6ccccc6n7-c6ccccc6)ccc54)c3)n2)cc1]

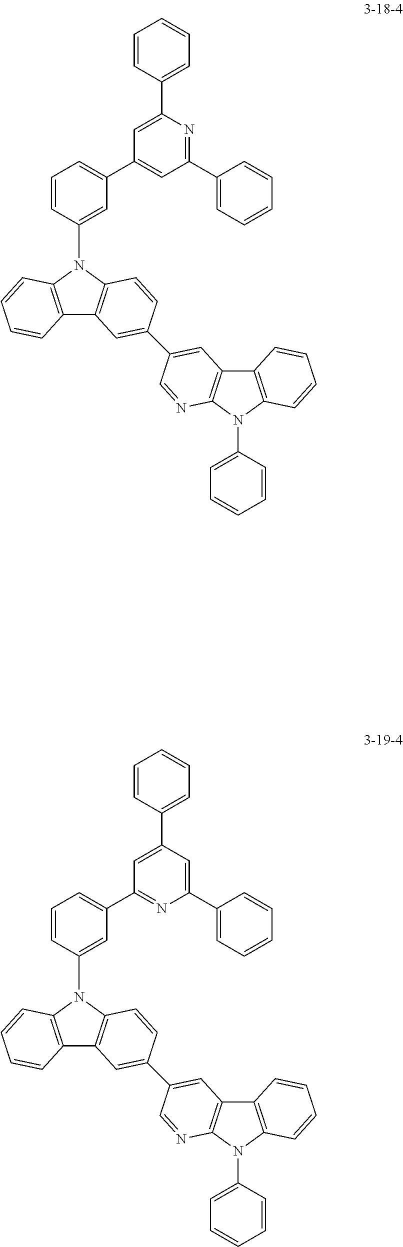 [SMILES: c1ccc(-c2cc(-c3cccc(-n4c5ccccc5c5cc(-c6cnc7c(c6)c6ccccc6n7-c6ccccc6)ccc54)c3)cc(-c3ccccc3)n2)cc1 ; c1ccc(-c2cc(-c3ccccc3)nc(-c3cccc(-n4c5ccccc5c5cc(-c6cnc7c(c6)c6ccccc6n7-c6ccccc6)ccc54)c3)c2)cc1]

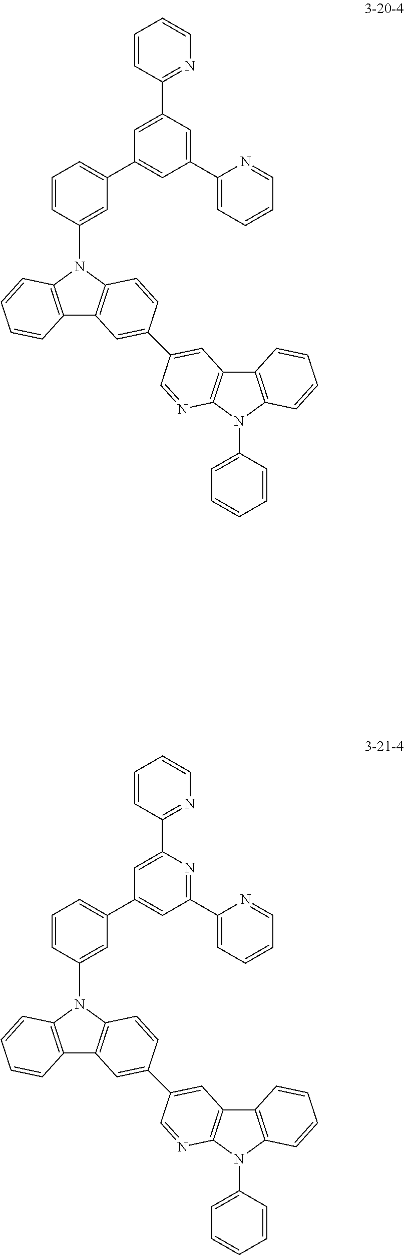 [SMILES: c1ccc(-n2c3ccccc3c3cc(-c4ccc5c(c4)c4ccccc4n5-c4cccc(-c5cc(-c6ccccn6)cc(-c6ccccn6)c5)c4)cnc32)cc1 ; c1ccc(-n2c3ccccc3c3cc(-c4ccc5c(c4)c4ccccc4n5-c4cccc(-c5cc(-c6ccccn6)nc(-c6ccccn6)c5)c4)cnc32)cc1]

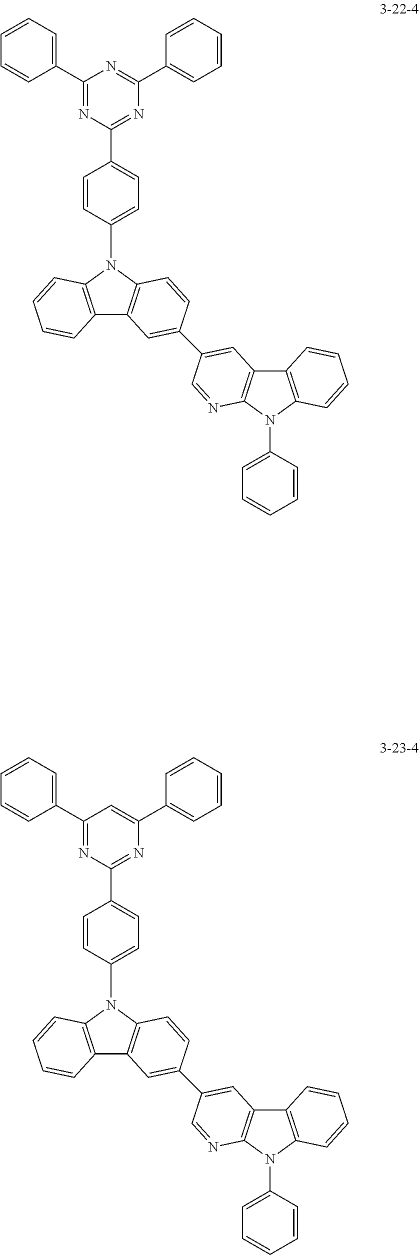 [SMILES: c1ccc(-c2nc(-c3ccccc3)nc(-c3ccc(-n4c5ccccc5c5cc(-c6cnc7c(c6)c6ccccc6n7-c6ccccc6)ccc54)cc3)n2)cc1 ; c1ccc(-c2cc(-c3ccccc3)nc(-c3ccc(-n4c5ccccc5c5cc(-c6cnc7c(c6)c6ccccc6n7-c6ccccc6)ccc54)cc3)n2)cc1]

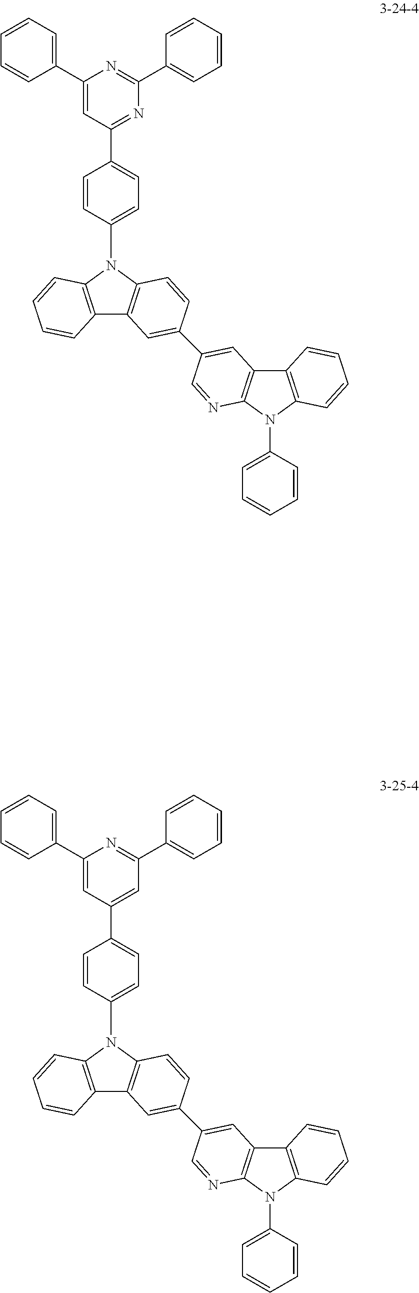 [SMILES: c1ccc(-c2cc(-c3ccc(-n4c5ccccc5c5cc(-c6cnc7c(c6)c6ccccc6n7-c6ccccc6)ccc54)cc3)nc(-c3ccccc3)n2)cc1 ; c1ccc(-c2cc(-c3ccc(-n4c5ccccc5c5cc(-c6cnc7c(c6)c6ccccc6n7-c6ccccc6)ccc54)cc3)cc(-c3ccccc3)n2)cc1]

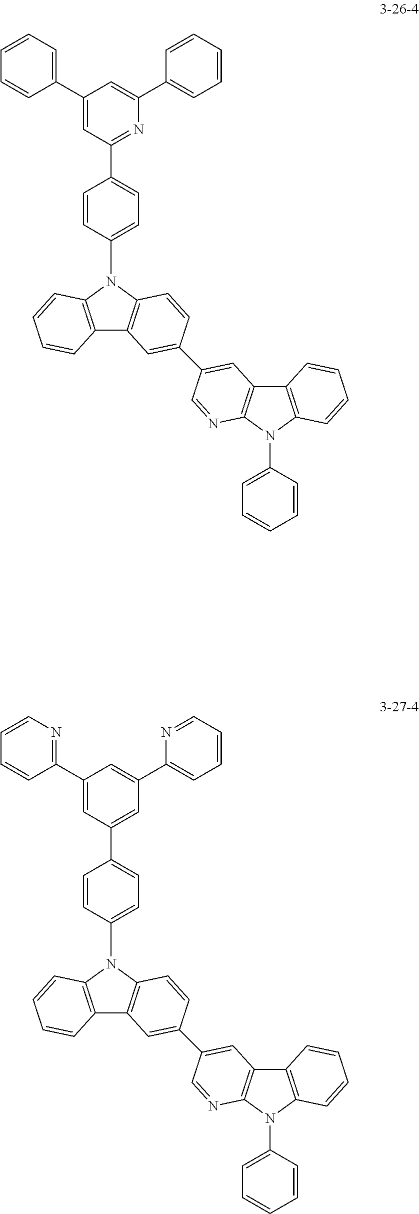 [SMILES: c1ccc(-c2cc(-c3ccccc3)nc(-c3ccc(-n4c5ccccc5c5cc(-c6cnc7c(c6)c6ccccc6n7-c6ccccc6)ccc54)cc3)c2)cc1 ; c1ccc(-n2c3ccccc3c3cc(-c4ccc5c(c4)c4ccccc4n5-c4ccc(-c5cc(-c6ccccn6)cc(-c6ccccn6)c5)cc4)cnc32)cc1]

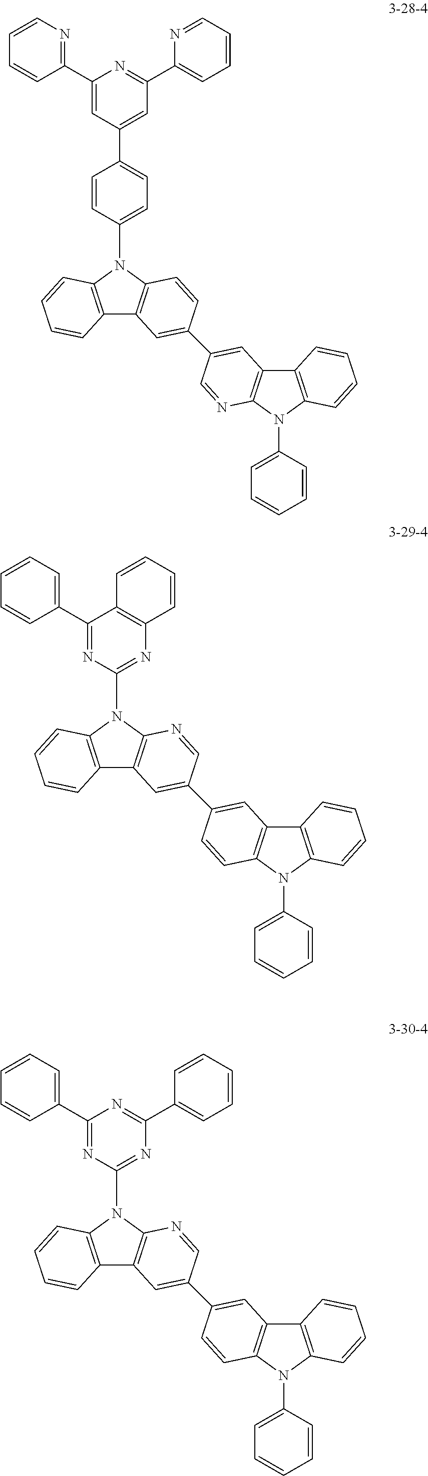 [SMILES: c1ccc(-n2c3ccccc3c3cc(-c4ccc5c(c4)c4ccccc4n5-c4ccc(-c5cc(-c6ccccn6)nc(-c6ccccn6)c5)cc4)cnc32)cc1 ; c1ccc(-c2nc(-n3c4ccccc4c4cc(-c5ccc6c(c5)c5ccccc5n6-c5ccccc5)cnc43)nc3ccccc23)cc1 ; c1ccc(-c2nc(-c3ccccc3)nc(-n3c4ccccc4c4cc(-c5ccc6c(c5)c5ccccc5n6-c5ccccc5)cnc43)n2)cc1]

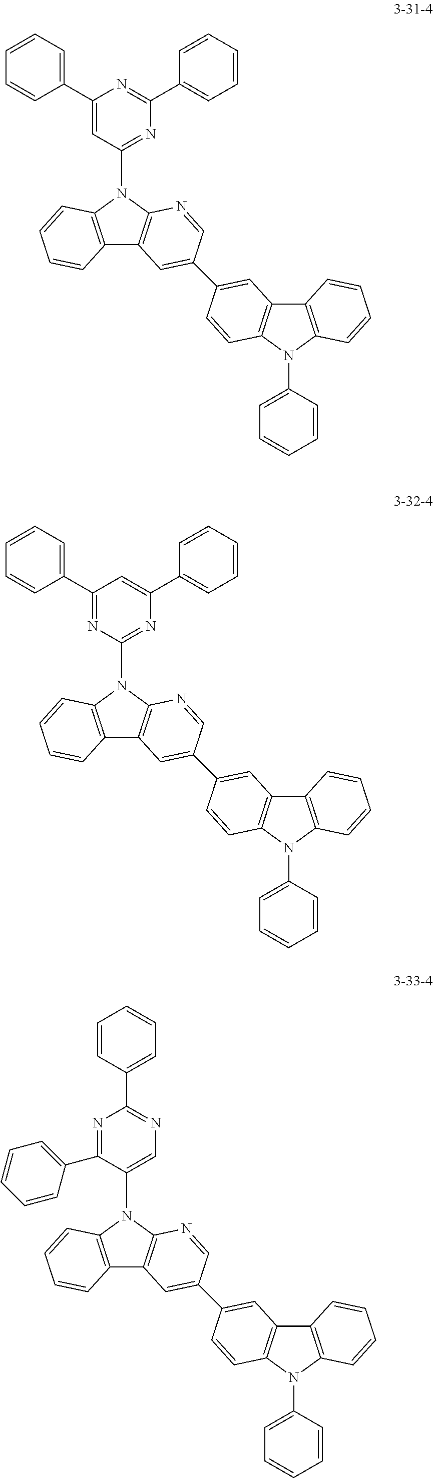 [SMILES: c1ccc(-c2cc(-n3c4ccccc4c4cc(-c5ccc6c(c5)c5ccccc5n6-c5ccccc5)cnc43)nc(-c3ccccc3)n2)cc1 ; c1ccc(-c2cc(-c3ccccc3)nc(-n3c4ccccc4c4cc(-c5ccc6c(c5)c5ccccc5n6-c5ccccc5)cnc43)n2)cc1 ; c1ccc(-c2ncc(-n3c4ccccc4c4cc(-c5ccc6c(c5)c5ccccc5n6-c5ccccc5)cnc43)c(-c3ccccc3)n2)cc1]

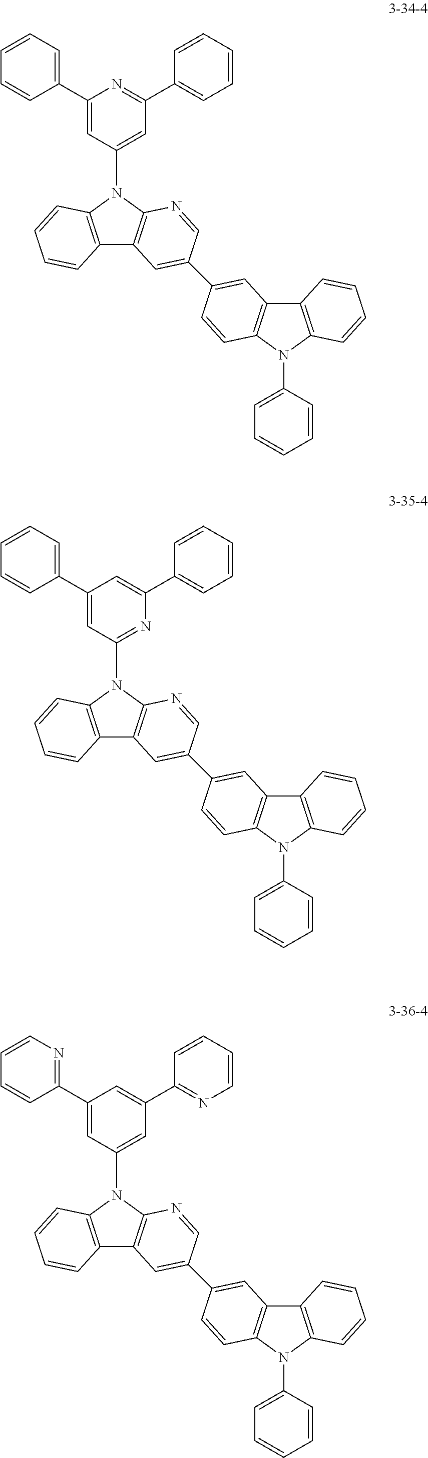 [SMILES: c1ccc(-c2cc(-n3c4ccccc4c4cc(-c5ccc6c(c5)c5ccccc5n6-c5ccccc5)cnc43)cc(-c3ccccc3)n2)cc1 ; c1ccc(-c2cc(-c3ccccc3)nc(-n3c4ccccc4c4cc(-c5ccc6c(c5)c5ccccc5n6-c5ccccc5)cnc43)c2)cc1 ; c1ccc(-n2c3ccccc3c3cc(-c4cnc5c(c4)c4ccccc4n5-c4cc(-c5ccccn5)cc(-c5ccccn5)c4)ccc32)cc1]

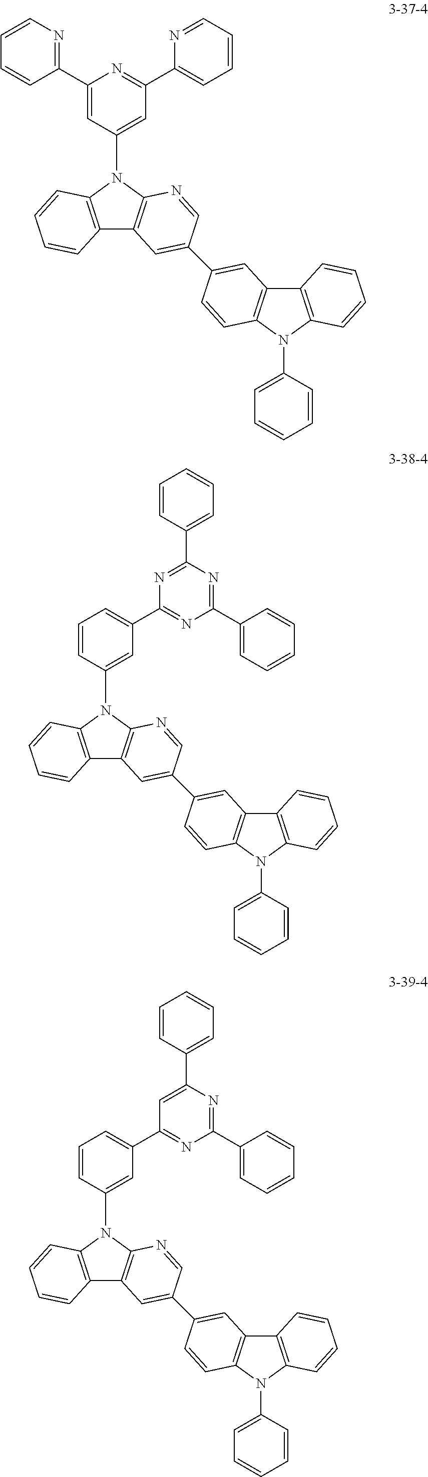 [SMILES: c1ccc(-n2c3ccccc3c3cc(-c4cnc5c(c4)c4ccccc4n5-c4cc(-c5ccccn5)nc(-c5ccccn5)c4)ccc32)cc1 ; c1ccc(-c2nc(-c3ccccc3)nc(-c3cccc(-n4c5ccccc5c5cc(-c6ccc7c(c6)c6ccccc6n7-c6ccccc6)cnc54)c3)n2)cc1 ; c1ccc(-c2cc(-c3cccc(-n4c5ccccc5c5cc(-c6ccc7c(c6)c6ccccc6n7-c6ccccc6)cnc54)c3)nc(-c3ccccc3)n2)cc1]

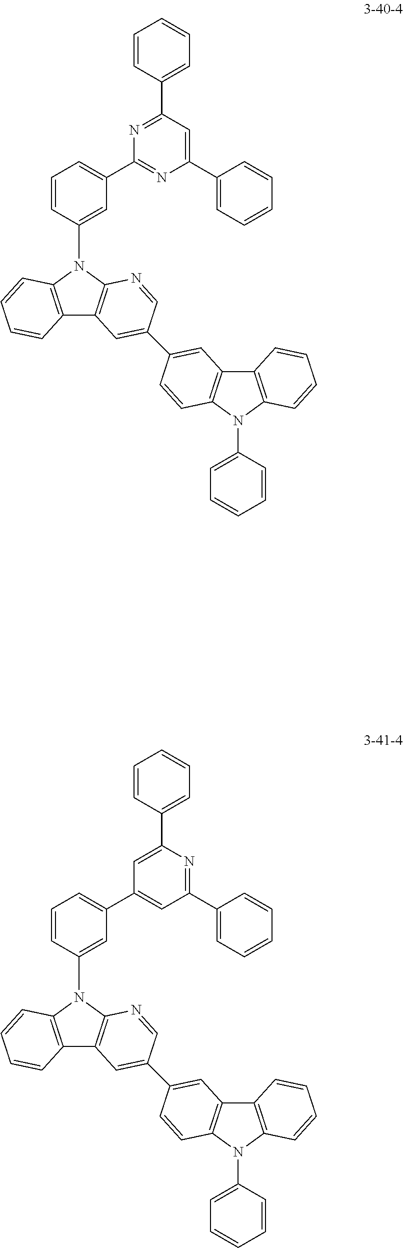 [SMILES: c1ccc(-c2cc(-c3ccccc3)nc(-c3cccc(-n4c5ccccc5c5cc(-c6ccc7c(c6)c6ccccc6n7-c6ccccc6)cnc54)c3)n2)cc1 ; c1ccc(-c2cc(-c3cccc(-n4c5ccccc5c5cc(-c6ccc7c(c6)c6ccccc6n7-c6ccccc6)cnc54)c3)cc(-c3ccccc3)n2)cc1]

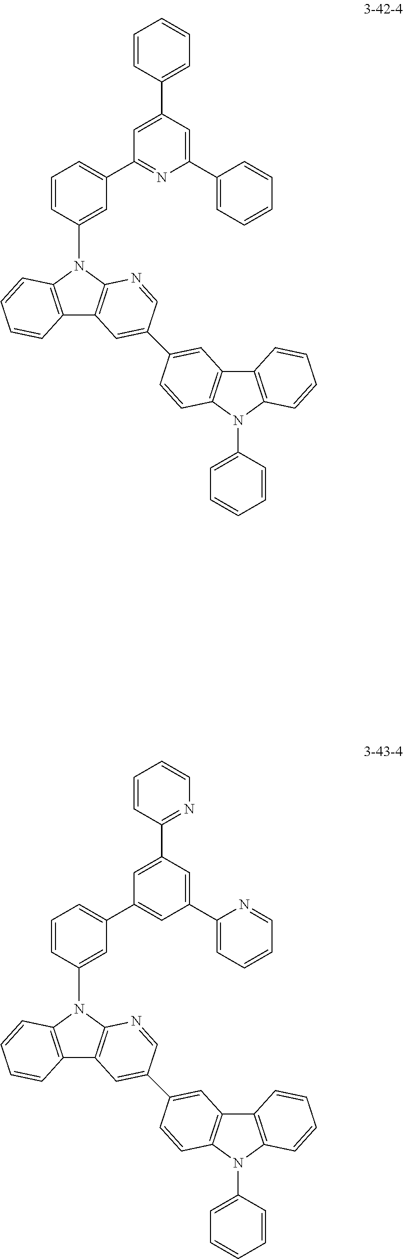 [SMILES: c1ccc(-c2cc(-c3ccccc3)nc(-c3cccc(-n4c5ccccc5c5cc(-c6ccc7c(c6)c6ccccc6n7-c6ccccc6)cnc54)c3)c2)cc1 ; c1ccc(-n2c3ccccc3c3cc(-c4cnc5c(c4)c4ccccc4n5-c4cccc(-c5cc(-c6ccccn6)cc(-c6ccccn6)c5)c4)ccc32)cc1]

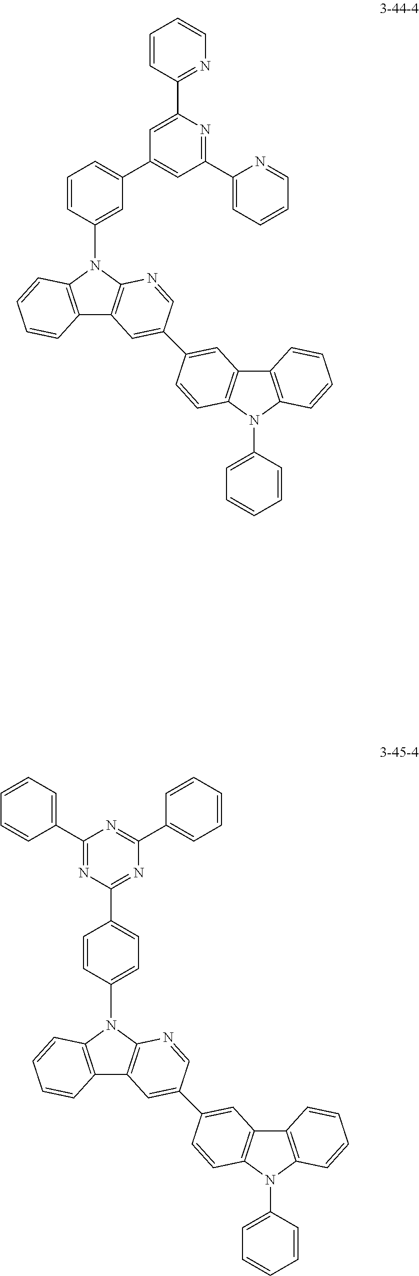 [SMILES: c1ccc(-n2c3ccccc3c3cc(-c4cnc5c(c4)c4ccccc4n5-c4cccc(-c5cc(-c6ccccn6)nc(-c6ccccn6)c5)c4)ccc32)cc1 ; c1ccc(-c2nc(-c3ccccc3)nc(-c3ccc(-n4c5ccccc5c5cc(-c6ccc7c(c6)c6ccccc6n7-c6ccccc6)cnc54)cc3)n2)cc1]

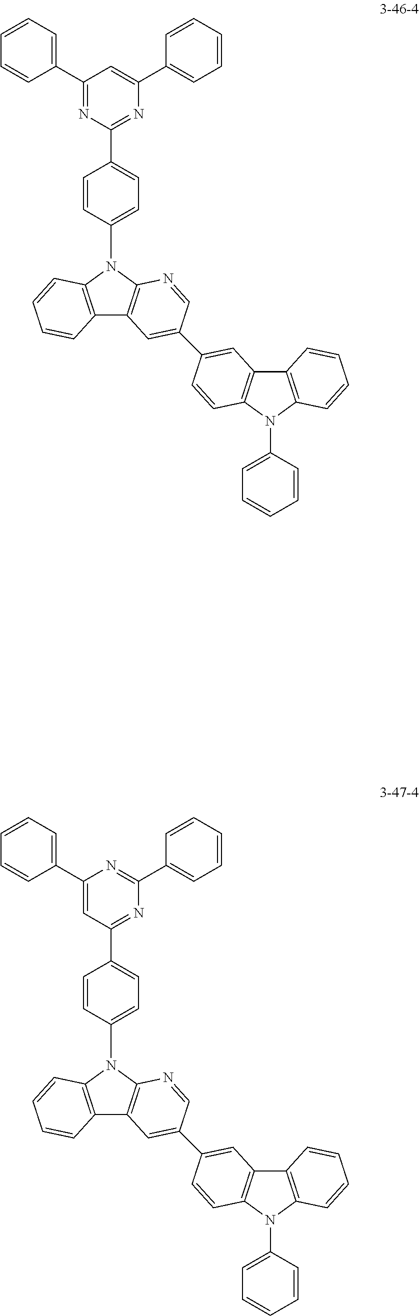 [SMILES: c1ccc(-c2cc(-c3ccccc3)nc(-c3ccc(-n4c5ccccc5c5cc(-c6ccc7c(c6)c6ccccc6n7-c6ccccc6)cnc54)cc3)n2)cc1 ; c1ccc(-c2cc(-c3ccc(-n4c5ccccc5c5cc(-c6ccc7c(c6)c6ccccc6n7-c6ccccc6)cnc54)cc3)nc(-c3ccccc3)n2)cc1]

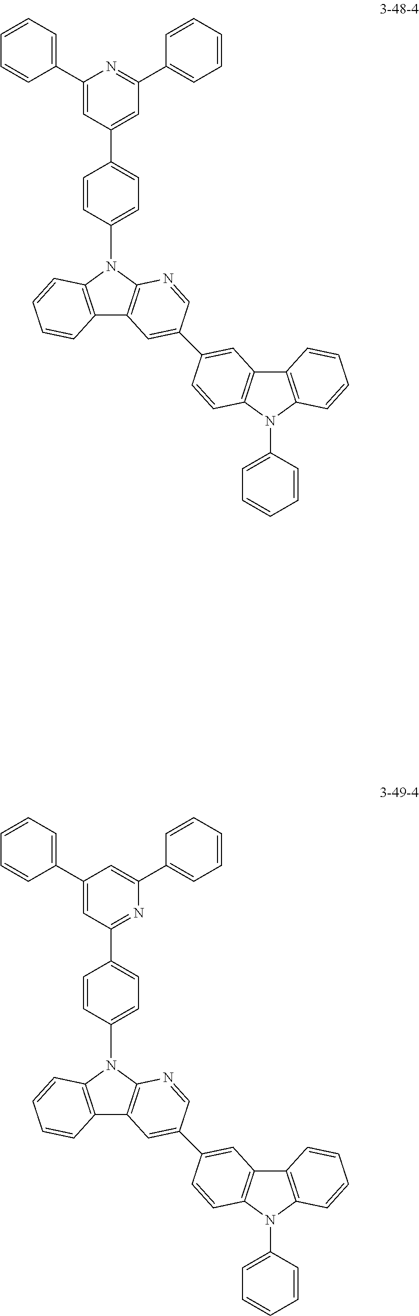 [SMILES: c1ccc(-c2cc(-c3ccc(-n4c5ccccc5c5cc(-c6ccc7c(c6)c6ccccc6n7-c6ccccc6)cnc54)cc3)cc(-c3ccccc3)n2)cc1 ; c1ccc(-c2cc(-c3ccccc3)nc(-c3ccc(-n4c5ccccc5c5cc(-c6ccc7c(c6)c6ccccc6n7-c6ccccc6)cnc54)cc3)c2)cc1]

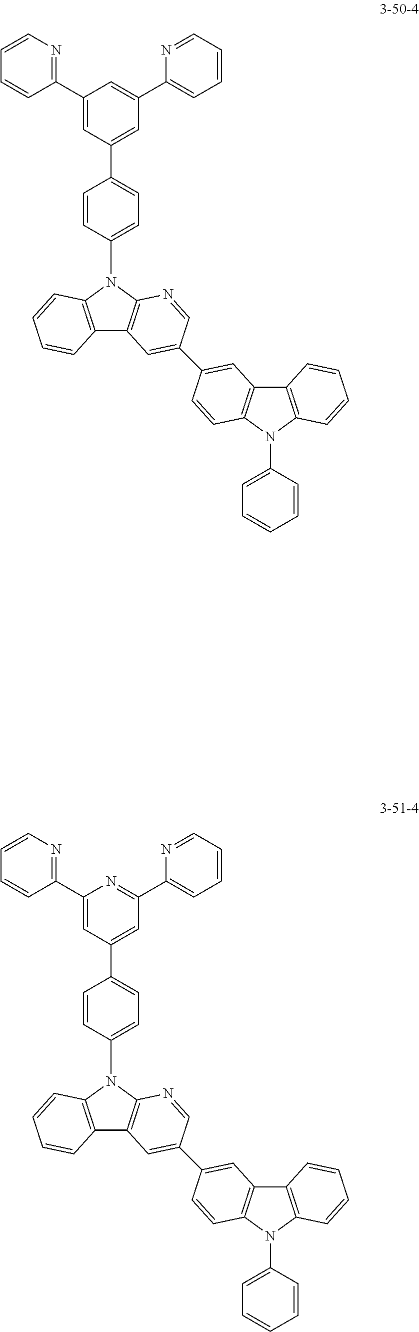 [SMILES: c1ccc(-n2c3ccccc3c3cc(-c4cnc5c(c4)c4ccccc4n5-c4ccc(-c5cc(-c6ccccn6)cc(-c6ccccn6)c5)cc4)ccc32)cc1 ; c1ccc(-n2c3ccccc3c3cc(-c4cnc5c(c4)c4ccccc4n5-c4ccc(-c5cc(-c6ccccn6)nc(-c6ccccn6)c5)cc4)ccc32)cc1]

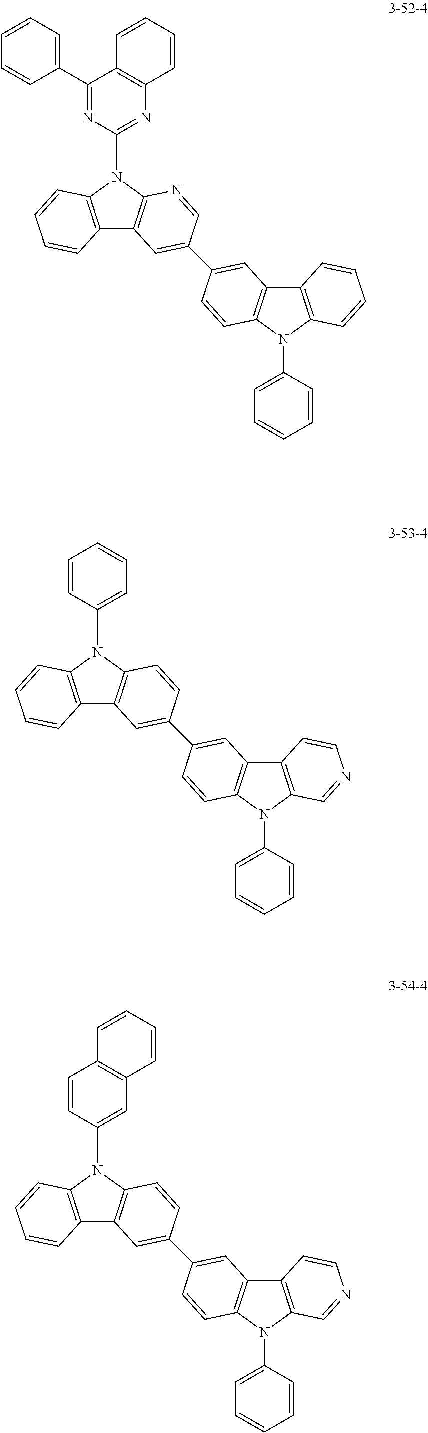 [SMILES: c1ccc(-c2nc(-n3c4ccccc4c4cc(-c5ccc6c(c5)c5ccccc5n6-c5ccccc5)cnc43)nc3ccccc23)cc1 ; c1ccc(-n2c3ccccc3c3cc(-c4ccc5c(c4)c4ccncc4n5-c4ccccc4)ccc32)cc1 ; c1ccc(-n2c3ccc(-c4ccc5c(c4)c4ccccc4n5-c4ccc5ccccc5c4)cc3c3ccncc32)cc1]

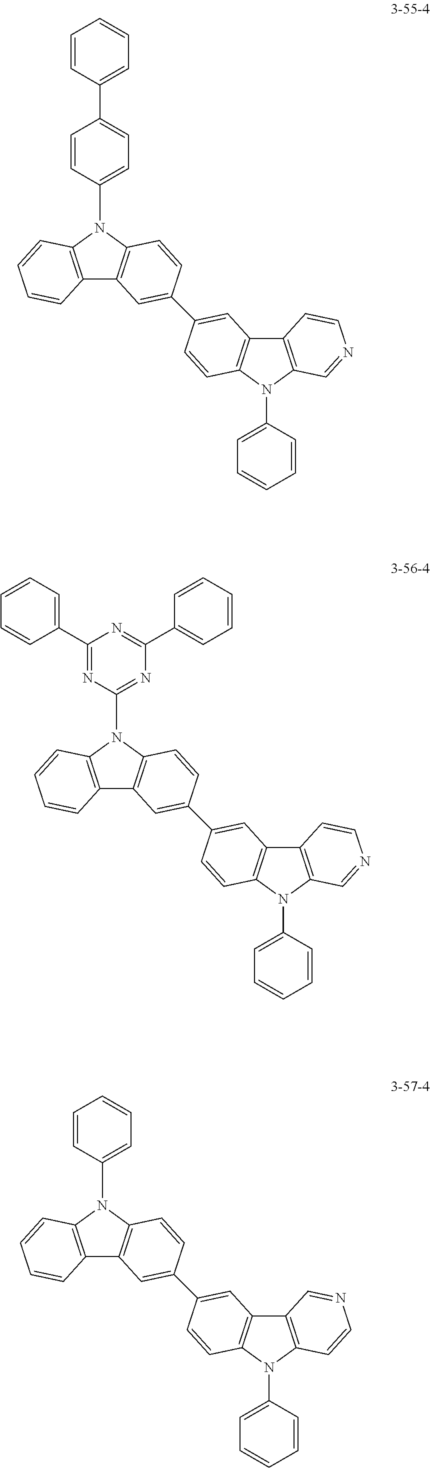 [SMILES: c1ccc(-c2ccc(-n3c4ccccc4c4cc(-c5ccc6c(c5)c5ccncc5n6-c5ccccc5)ccc43)cc2)cc1 ; c1ccc(-c2nc(-c3ccccc3)nc(-n3c4ccccc4c4cc(-c5ccc6c(c5)c5ccncc5n6-c5ccccc5)ccc43)n2)cc1 ; c1ccc(-n2c3ccccc3c3cc(-c4ccc5c(c4)c4cnccc4n5-c4ccccc4)ccc32)cc1]

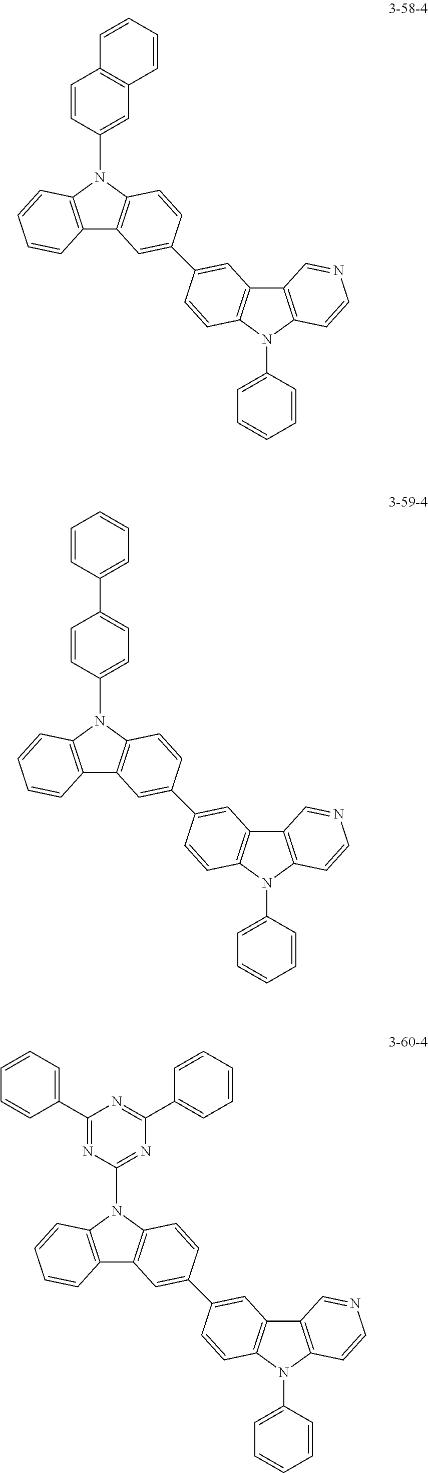 [SMILES: c1ccc(-n2c3ccncc3c3cc(-c4ccc5c(c4)c4ccccc4n5-c4ccc5ccccc5c4)ccc32)cc1 ; c1ccc(-c2ccc(-n3c4ccccc4c4cc(-c5ccc6c(c5)c5cnccc5n6-c5ccccc5)ccc43)cc2)cc1 ; c1ccc(-c2nc(-c3ccccc3)nc(-n3c4ccccc4c4cc(-c5ccc6c(c5)c5cnccc5n6-c5ccccc5)ccc43)n2)cc1]

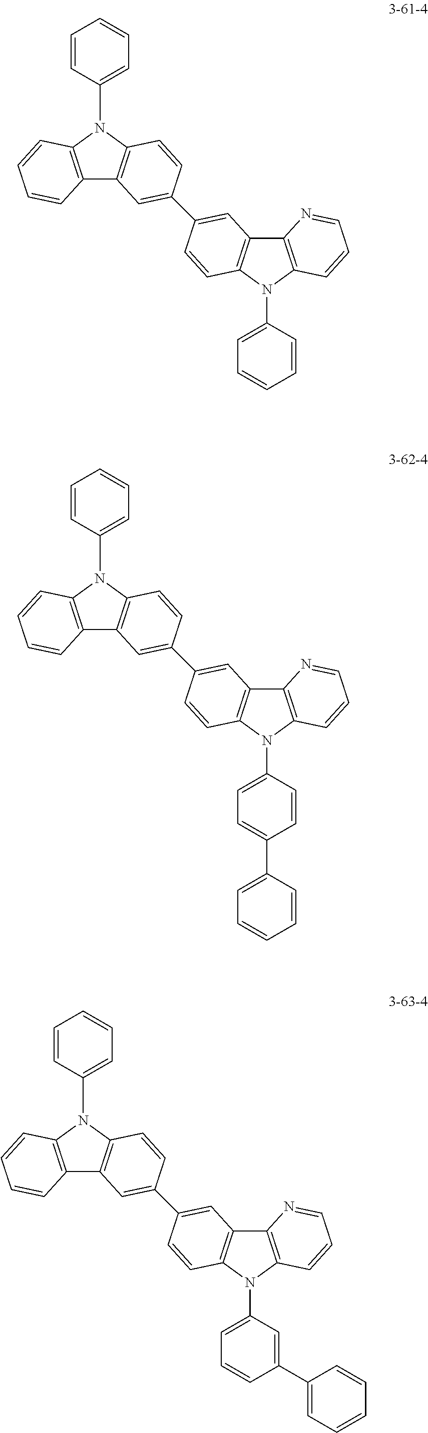 [SMILES: c1ccc(-n2c3ccccc3c3cc(-c4ccc5c(c4)c4ncccc4n5-c4ccccc4)ccc32)cc1 ; c1ccc(-c2ccc(-n3c4ccc(-c5ccc6c(c5)c5ccccc5n6-c5ccccc5)cc4c4ncccc43)cc2)cc1 ; c1ccc(-c2cccc(-n3c4ccc(-c5ccc6c(c5)c5ccccc5n6-c5ccccc5)cc4c4ncccc43)c2)cc1]

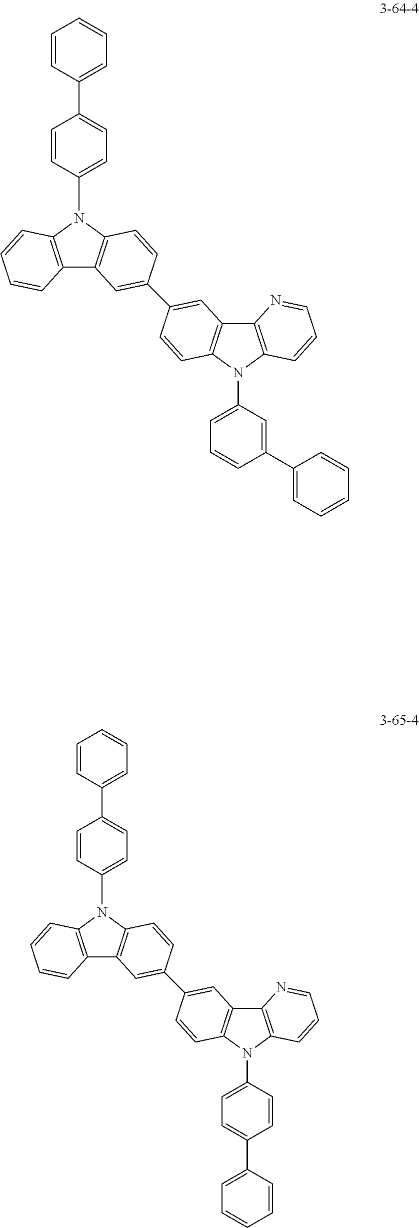 [SMILES: c1ccc(-c2ccc(-n3c4ccccc4c4cc(-c5ccc6c(c5)c5ncccc5n6-c5cccc(-c6ccccc6)c5)ccc43)cc2)cc1 ; c1ccc(-c2ccc(-n3c4ccccc4c4cc(-c5ccc6c(c5)c5ncccc5n6-c5ccc(-c6ccccc6)cc5)ccc43)cc2)cc1]

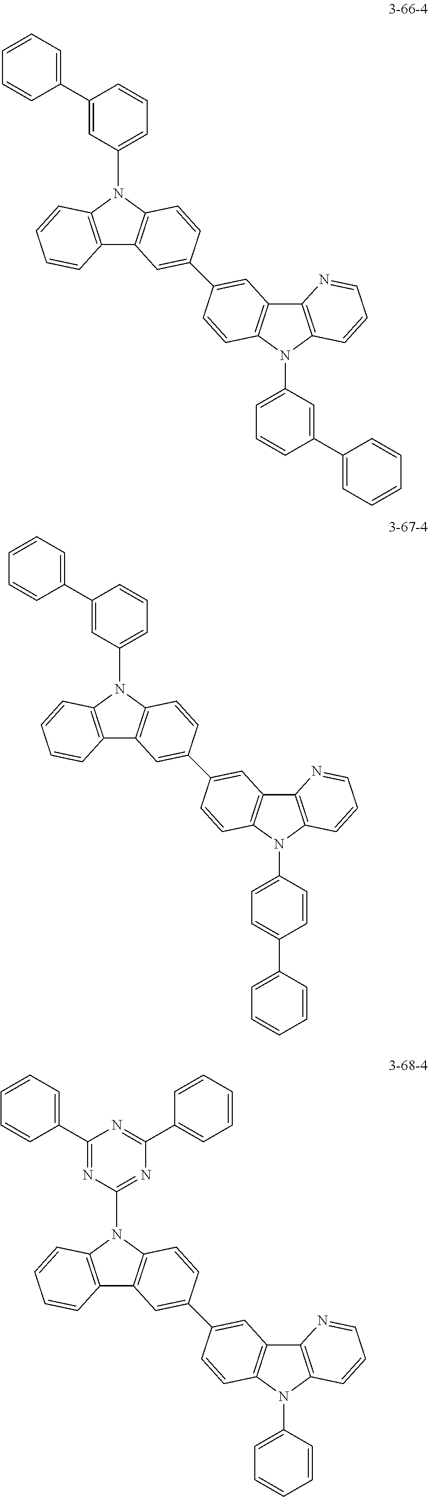 [SMILES: c1ccc(-c2cccc(-n3c4ccccc4c4cc(-c5ccc6c(c5)c5ncccc5n6-c5cccc(-c6ccccc6)c5)ccc43)c2)cc1 ; c1ccc(-c2ccc(-n3c4ccc(-c5ccc6c(c5)c5ccccc5n6-c5cccc(-c6ccccc6)c5)cc4c4ncccc43)cc2)cc1 ; c1ccc(-c2nc(-c3ccccc3)nc(-n3c4ccccc4c4cc(-c5ccc6c(c5)c5ncccc5n6-c5ccccc5)ccc43)n2)cc1]

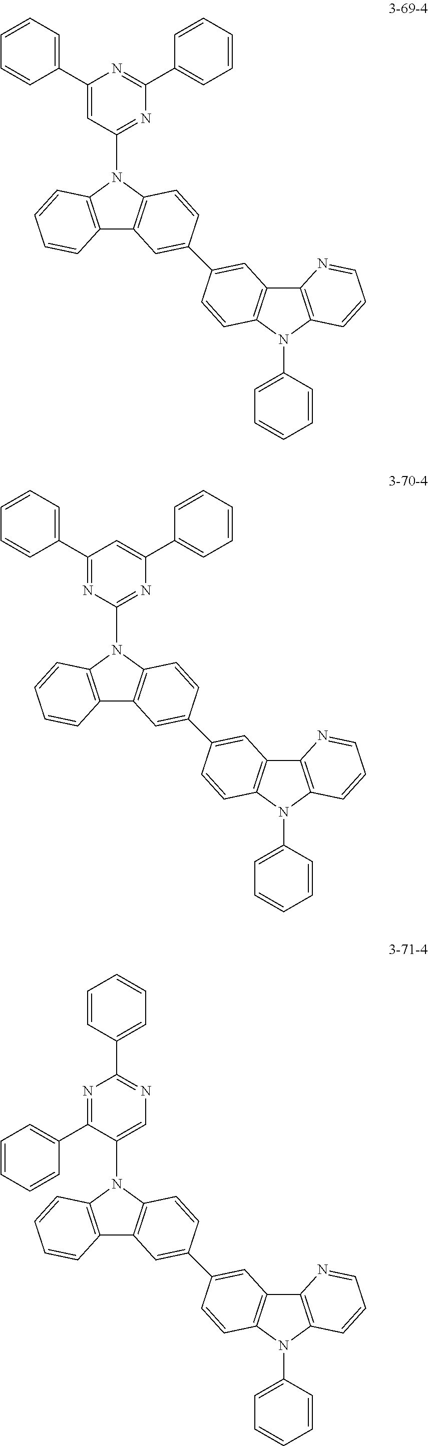 [SMILES: c1ccc(-c2cc(-n3c4ccccc4c4cc(-c5ccc6c(c5)c5ncccc5n6-c5ccccc5)ccc43)nc(-c3ccccc3)n2)cc1 ; c1ccc(-c2cc(-c3ccccc3)nc(-n3c4ccccc4c4cc(-c5ccc6c(c5)c5ncccc5n6-c5ccccc5)ccc43)n2)cc1 ; c1ccc(-c2ncc(-n3c4ccccc4c4cc(-c5ccc6c(c5)c5ncccc5n6-c5ccccc5)ccc43)c(-c3ccccc3)n2)cc1]

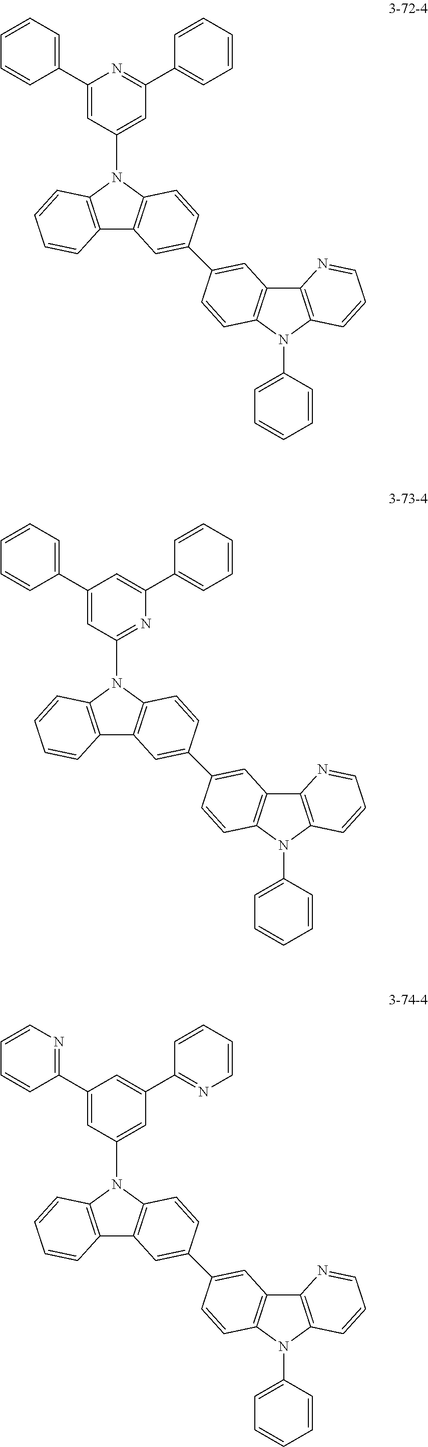 [SMILES: c1ccc(-c2cc(-n3c4ccccc4c4cc(-c5ccc6c(c5)c5ncccc5n6-c5ccccc5)ccc43)cc(-c3ccccc3)n2)cc1 ; c1ccc(-c2cc(-c3ccccc3)nc(-n3c4ccccc4c4cc(-c5ccc6c(c5)c5ncccc5n6-c5ccccc5)ccc43)c2)cc1 ; c1ccc(-n2c3ccc(-c4ccc5c(c4)c4ccccc4n5-c4cc(-c5ccccn5)cc(-c5ccccn5)c4)cc3c3ncccc32)cc1]

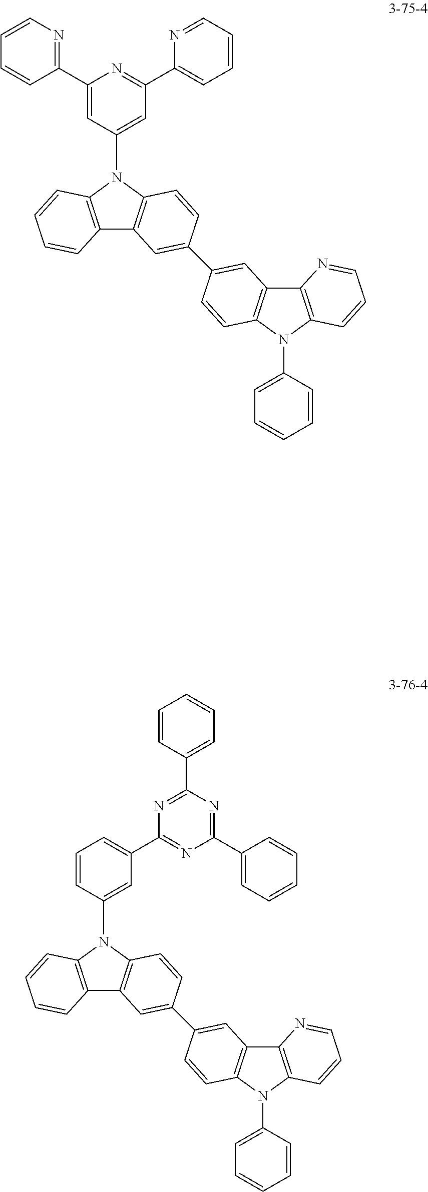 [SMILES: c1ccc(-n2c3ccc(-c4ccc5c(c4)c4ccccc4n5-c4cc(-c5ccccn5)nc(-c5ccccn5)c4)cc3c3ncccc32)cc1 ; c1ccc(-c2nc(-c3ccccc3)nc(-c3cccc(-n4c5ccccc5c5cc(-c6ccc7c(c6)c6ncccc6n7-c6ccccc6)ccc54)c3)n2)cc1]

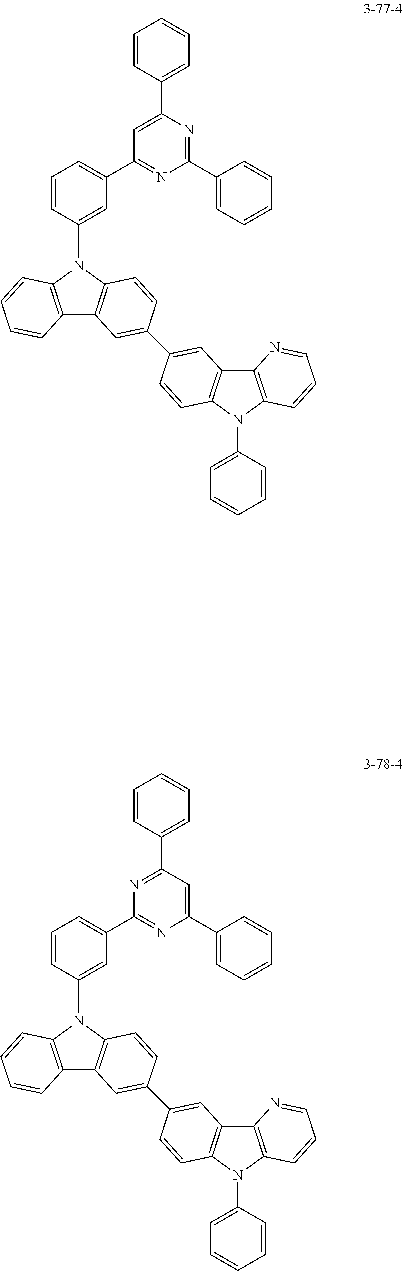 [SMILES: c1ccc(-c2cc(-c3cccc(-n4c5ccccc5c5cc(-c6ccc7c(c6)c6ncccc6n7-c6ccccc6)ccc54)c3)nc(-c3ccccc3)n2)cc1 ; c1ccc(-c2cc(-c3ccccc3)nc(-c3cccc(-n4c5ccccc5c5cc(-c6ccc7c(c6)c6ncccc6n7-c6ccccc6)ccc54)c3)n2)cc1]

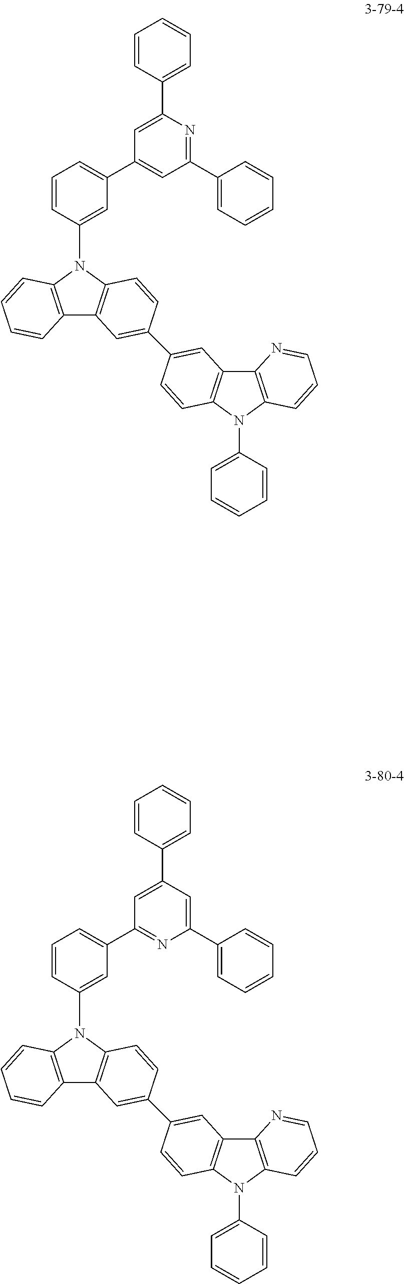 [SMILES: c1ccc(-c2cc(-c3cccc(-n4c5ccccc5c5cc(-c6ccc7c(c6)c6ncccc6n7-c6ccccc6)ccc54)c3)cc(-c3ccccc3)n2)cc1 ; c1ccc(-c2cc(-c3ccccc3)nc(-c3cccc(-n4c5ccccc5c5cc(-c6ccc7c(c6)c6ncccc6n7-c6ccccc6)ccc54)c3)c2)cc1]

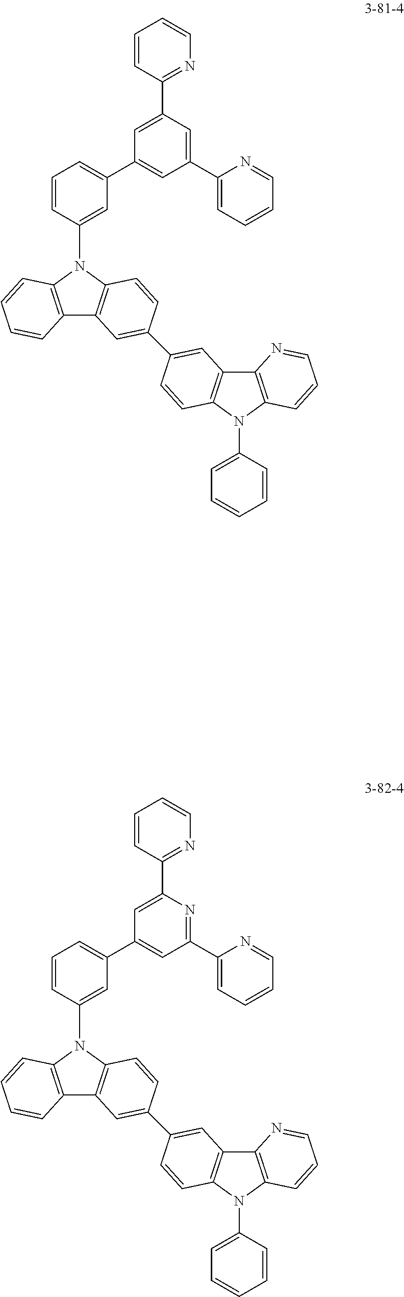 [SMILES: c1ccc(-n2c3ccc(-c4ccc5c(c4)c4ccccc4n5-c4cccc(-c5cc(-c6ccccn6)cc(-c6ccccn6)c5)c4)cc3c3ncccc32)cc1 ; c1ccc(-n2c3ccc(-c4ccc5c(c4)c4ccccc4n5-c4cccc(-c5cc(-c6ccccn6)nc(-c6ccccn6)c5)c4)cc3c3ncccc32)cc1]

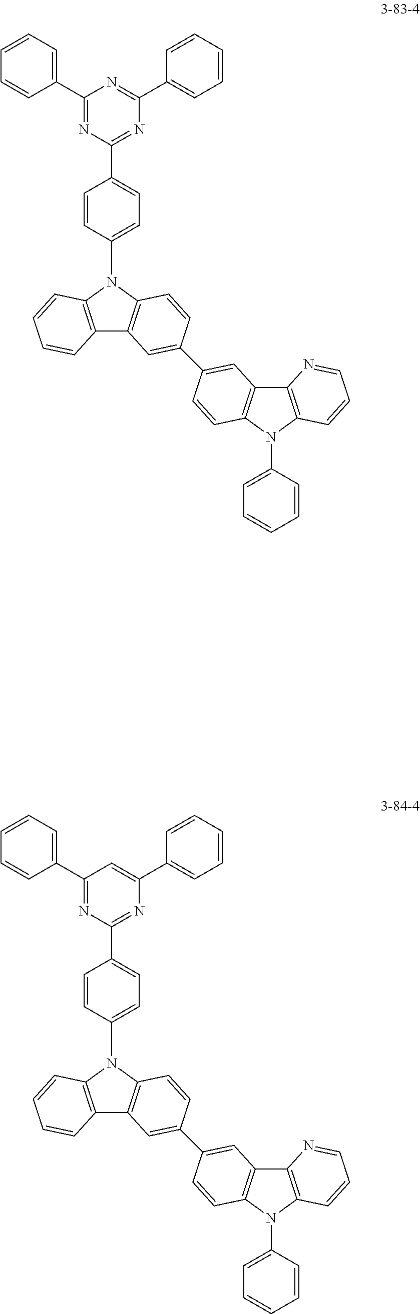 [SMILES: c1ccc(-c2nc(-c3ccccc3)nc(-c3ccc(-n4c5ccccc5c5cc(-c6ccc7c(c6)c6ncccc6n7-c6ccccc6)ccc54)cc3)n2)cc1 ; c1ccc(-c2cc(-c3ccccc3)nc(-c3ccc(-n4c5ccccc5c5cc(-c6ccc7c(c6)c6ncccc6n7-c6ccccc6)ccc54)cc3)n2)cc1]

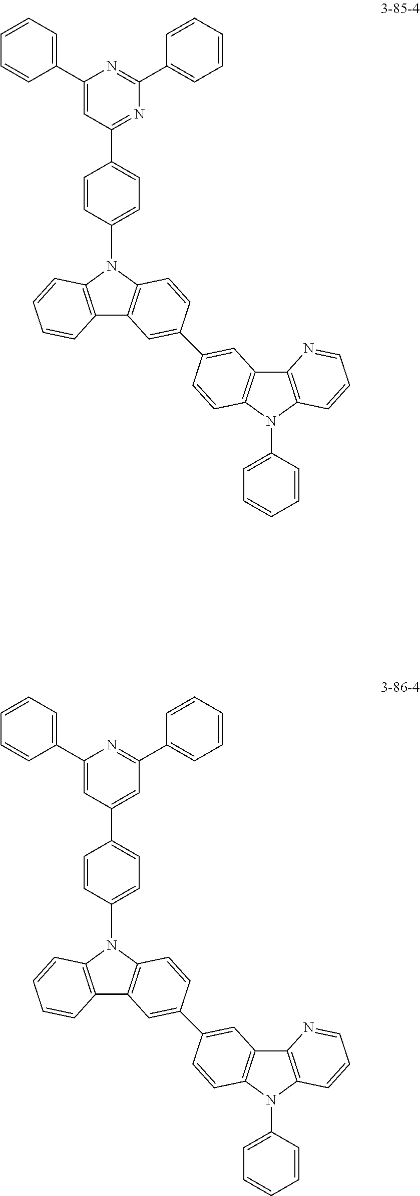 [SMILES: c1ccc(-c2cc(-c3ccc(-n4c5ccccc5c5cc(-c6ccc7c(c6)c6ncccc6n7-c6ccccc6)ccc54)cc3)nc(-c3ccccc3)n2)cc1 ; c1ccc(-c2cc(-c3ccc(-n4c5ccccc5c5cc(-c6ccc7c(c6)c6ncccc6n7-c6ccccc6)ccc54)cc3)cc(-c3ccccc3)n2)cc1]

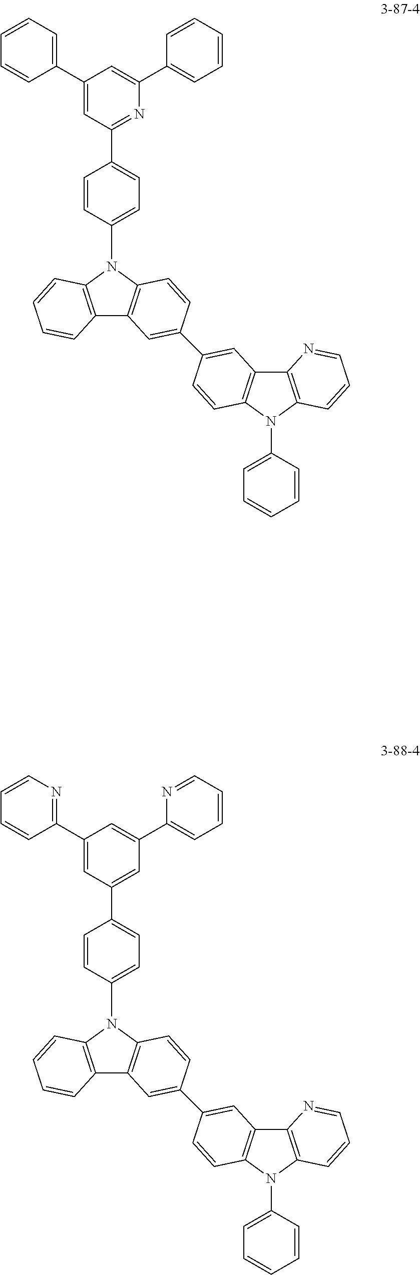 [SMILES: c1ccc(-c2cc(-c3ccccc3)nc(-c3ccc(-n4c5ccccc5c5cc(-c6ccc7c(c6)c6ncccc6n7-c6ccccc6)ccc54)cc3)c2)cc1 ; c1ccc(-n2c3ccc(-c4ccc5c(c4)c4ccccc4n5-c4ccc(-c5cc(-c6ccccn6)cc(-c6ccccn6)c5)cc4)cc3c3ncccc32)cc1]

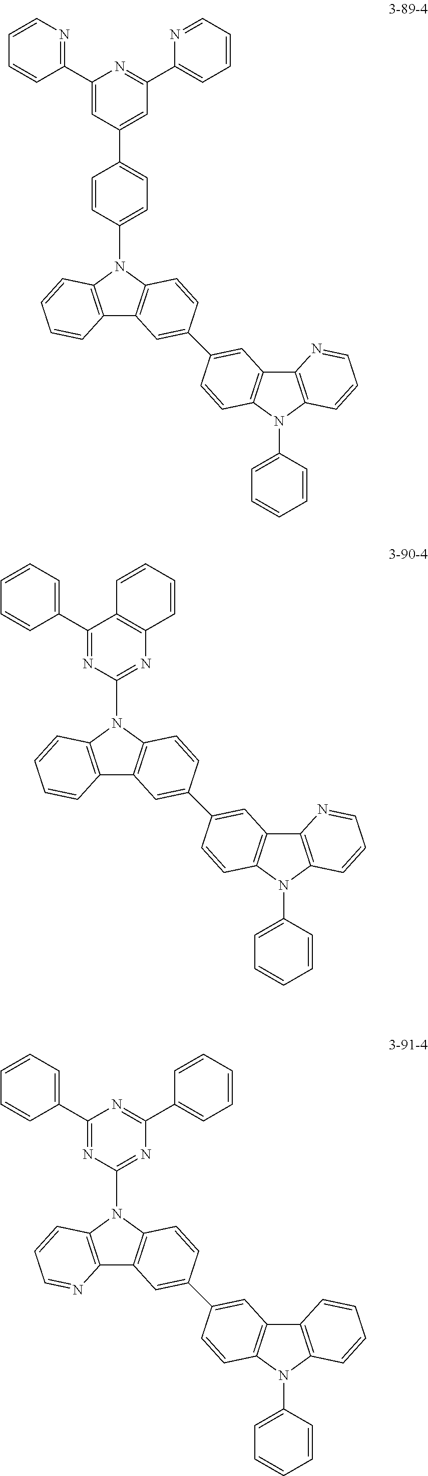 [SMILES: c1ccc(-n2c3ccc(-c4ccc5c(c4)c4ccccc4n5-c4ccc(-c5cc(-c6ccccn6)nc(-c6ccccn6)c5)cc4)cc3c3ncccc32)cc1 ; c1ccc(-c2nc(-n3c4ccccc4c4cc(-c5ccc6c(c5)c5ncccc5n6-c5ccccc5)ccc43)nc3ccccc23)cc1 ; c1ccc(-c2nc(-c3ccccc3)nc(-n3c4ccc(-c5ccc6c(c5)c5ccccc5n6-c5ccccc5)cc4c4ncccc43)n2)cc1]

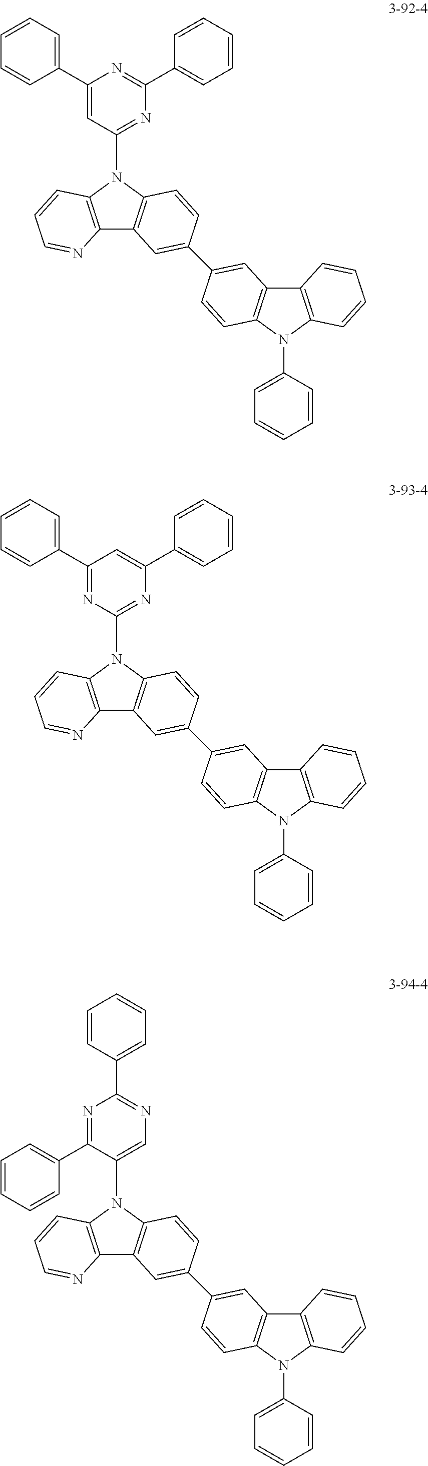 [SMILES: c1ccc(-c2cc(-n3c4ccc(-c5ccc6c(c5)c5ccccc5n6-c5ccccc5)cc4c4ncccc43)nc(-c3ccccc3)n2)cc1 ; c1ccc(-c2cc(-c3ccccc3)nc(-n3c4ccc(-c5ccc6c(c5)c5ccccc5n6-c5ccccc5)cc4c4ncccc43)n2)cc1 ; c1ccc(-c2ncc(-n3c4ccc(-c5ccc6c(c5)c5ccccc5n6-c5ccccc5)cc4c4ncccc43)c(-c3ccccc3)n2)cc1]

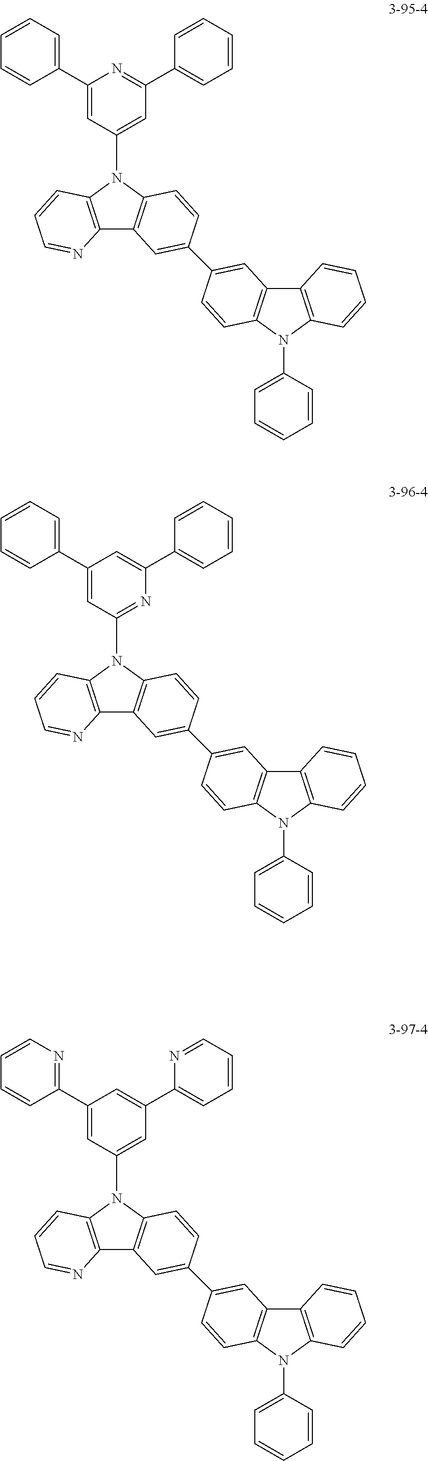 [SMILES: c1ccc(-c2cc(-n3c4ccc(-c5ccc6c(c5)c5ccccc5n6-c5ccccc5)cc4c4ncccc43)cc(-c3ccccc3)n2)cc1 ; c1ccc(-c2cc(-c3ccccc3)nc(-n3c4ccc(-c5ccc6c(c5)c5ccccc5n6-c5ccccc5)cc4c4ncccc43)c2)cc1 ; c1ccc(-n2c3ccccc3c3cc(-c4ccc5c(c4)c4ncccc4n5-c4cc(-c5ccccn5)cc(-c5ccccn5)c4)ccc32)cc1]

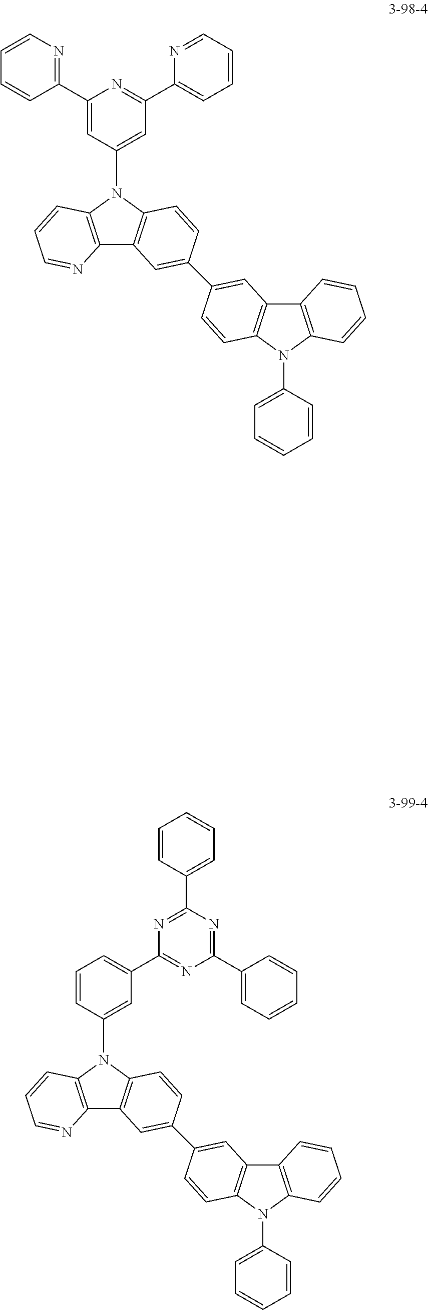 [SMILES: c1ccc(-n2c3ccccc3c3cc(-c4ccc5c(c4)c4ncccc4n5-c4cc(-c5ccccn5)nc(-c5ccccn5)c4)ccc32)cc1 ; c1ccc(-c2nc(-c3ccccc3)nc(-c3cccc(-n4c5ccc(-c6ccc7c(c6)c6ccccc6n7-c6ccccc6)cc5c5ncccc54)c3)n2)cc1]

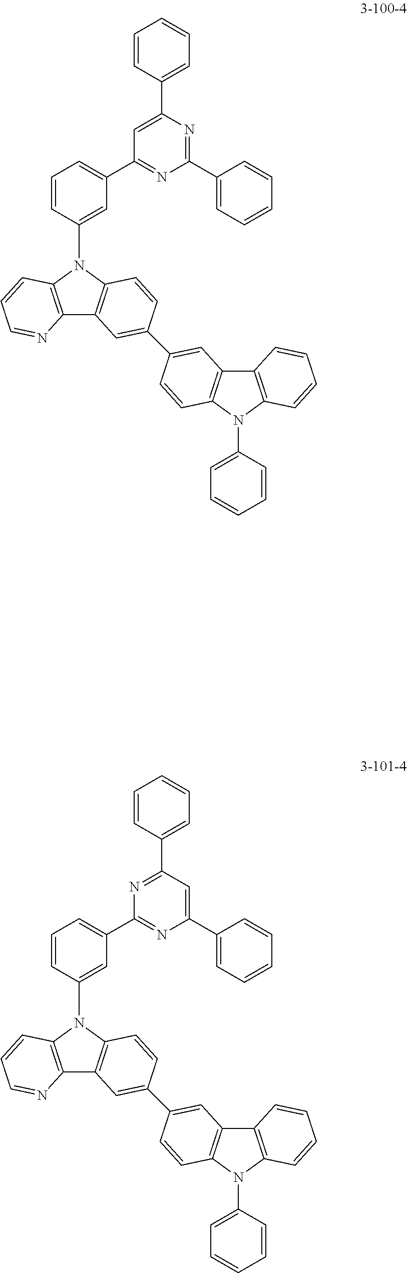 [SMILES: c1ccc(-c2cc(-c3cccc(-n4c5ccc(-c6ccc7c(c6)c6ccccc6n7-c6ccccc6)cc5c5ncccc54)c3)nc(-c3ccccc3)n2)cc1 ; c1ccc(-c2cc(-c3ccccc3)nc(-c3cccc(-n4c5ccc(-c6ccc7c(c6)c6ccccc6n7-c6ccccc6)cc5c5ncccc54)c3)n2)cc1]

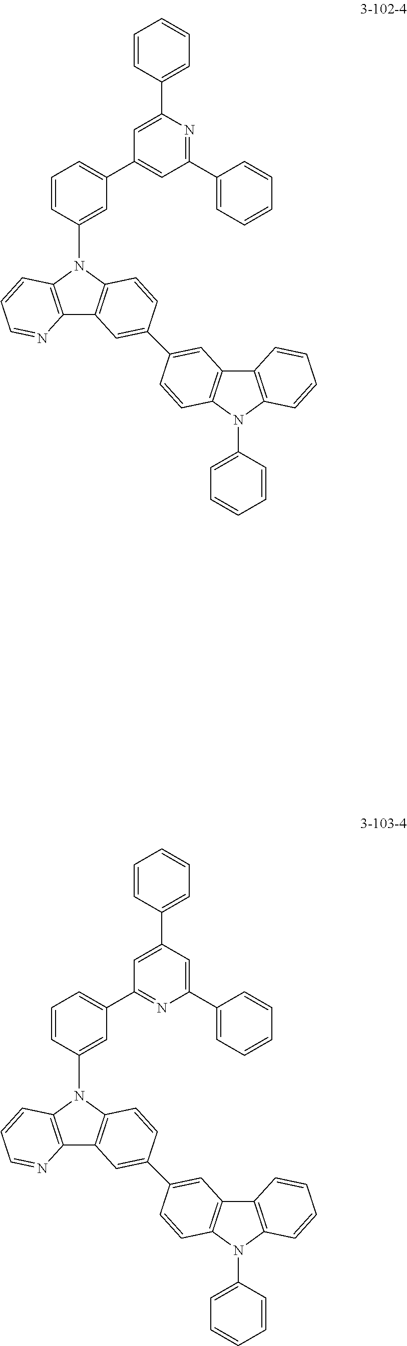 [SMILES: c1ccc(-c2cc(-c3cccc(-n4c5ccc(-c6ccc7c(c6)c6ccccc6n7-c6ccccc6)cc5c5ncccc54)c3)cc(-c3ccccc3)n2)cc1 ; c1ccc(-c2cc(-c3ccccc3)nc(-c3cccc(-n4c5ccc(-c6ccc7c(c6)c6ccccc6n7-c6ccccc6)cc5c5ncccc54)c3)c2)cc1]

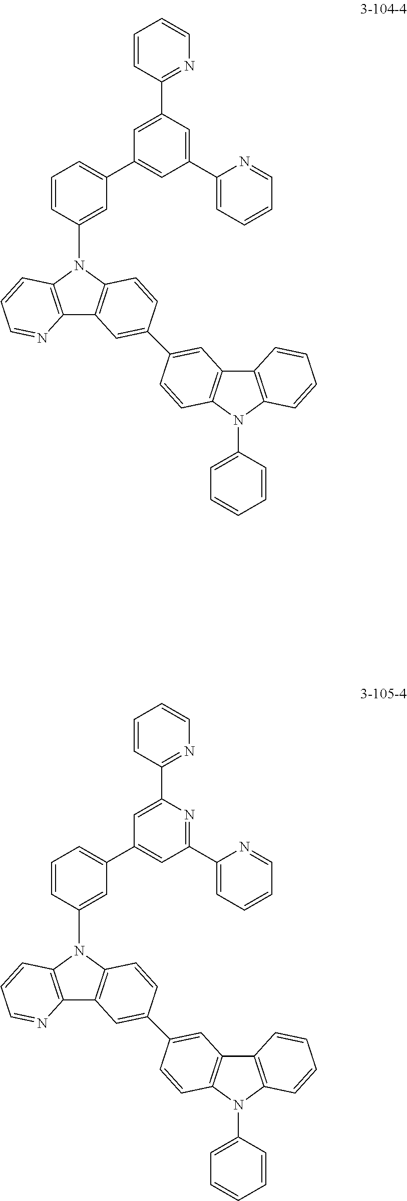 [SMILES: c1ccc(-n2c3ccccc3c3cc(-c4ccc5c(c4)c4ncccc4n5-c4cccc(-c5cc(-c6ccccn6)cc(-c6ccccn6)c5)c4)ccc32)cc1 ; c1ccc(-n2c3ccccc3c3cc(-c4ccc5c(c4)c4ncccc4n5-c4cccc(-c5cc(-c6ccccn6)nc(-c6ccccn6)c5)c4)ccc32)cc1]

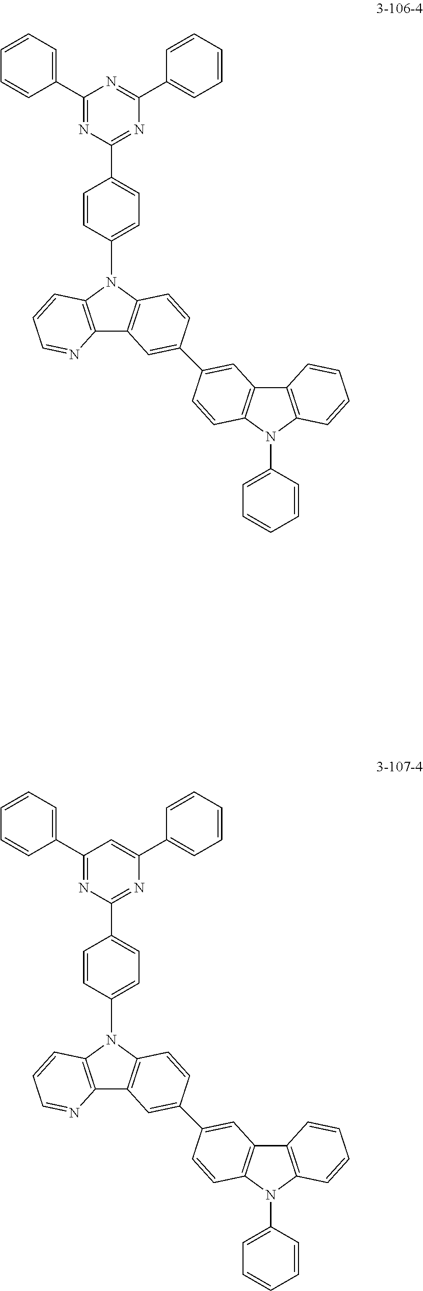 [SMILES: c1ccc(-c2nc(-c3ccccc3)nc(-c3ccc(-n4c5ccc(-c6ccc7c(c6)c6ccccc6n7-c6ccccc6)cc5c5ncccc54)cc3)n2)cc1 ; c1ccc(-c2cc(-c3ccccc3)nc(-c3ccc(-n4c5ccc(-c6ccc7c(c6)c6ccccc6n7-c6ccccc6)cc5c5ncccc54)cc3)n2)cc1]

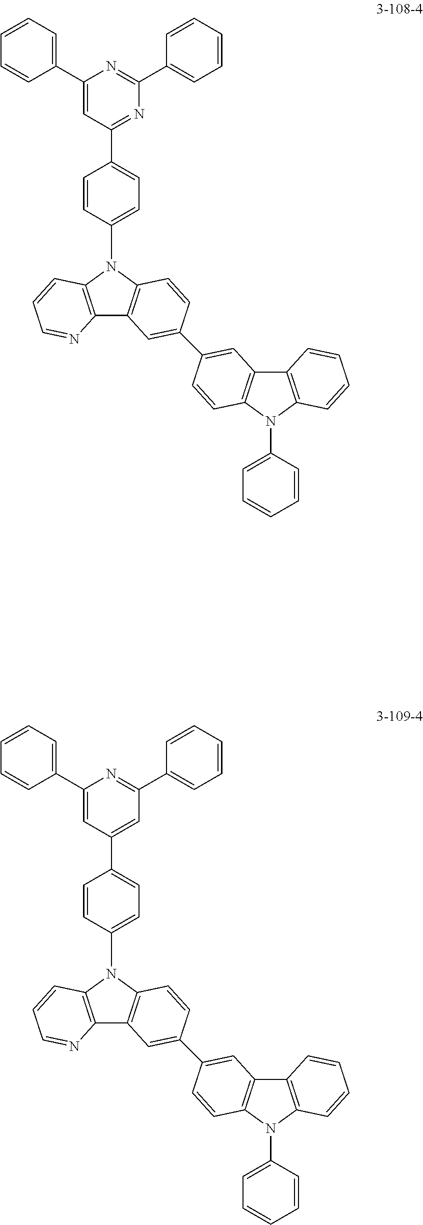 [SMILES: c1ccc(-c2cc(-c3ccc(-n4c5ccc(-c6ccc7c(c6)c6ccccc6n7-c6ccccc6)cc5c5ncccc54)cc3)nc(-c3ccccc3)n2)cc1 ; c1ccc(-c2cc(-c3ccc(-n4c5ccc(-c6ccc7c(c6)c6ccccc6n7-c6ccccc6)cc5c5ncccc54)cc3)cc(-c3ccccc3)n2)cc1]

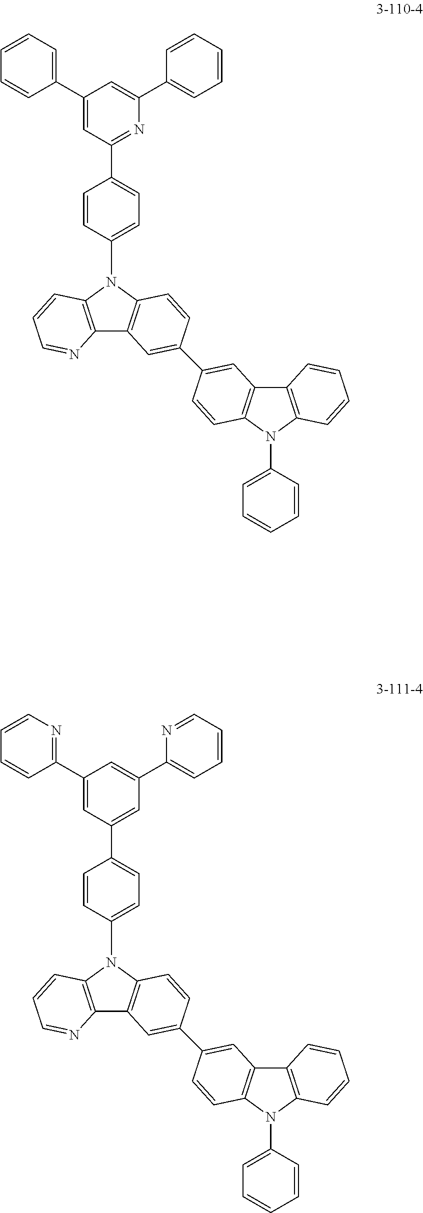 [SMILES: c1ccc(-c2cc(-c3ccccc3)nc(-c3ccc(-n4c5ccc(-c6ccc7c(c6)c6ccccc6n7-c6ccccc6)cc5c5ncccc54)cc3)c2)cc1 ; c1ccc(-n2c3ccccc3c3cc(-c4ccc5c(c4)c4ncccc4n5-c4ccc(-c5cc(-c6ccccn6)cc(-c6ccccn6)c5)cc4)ccc32)cc1]

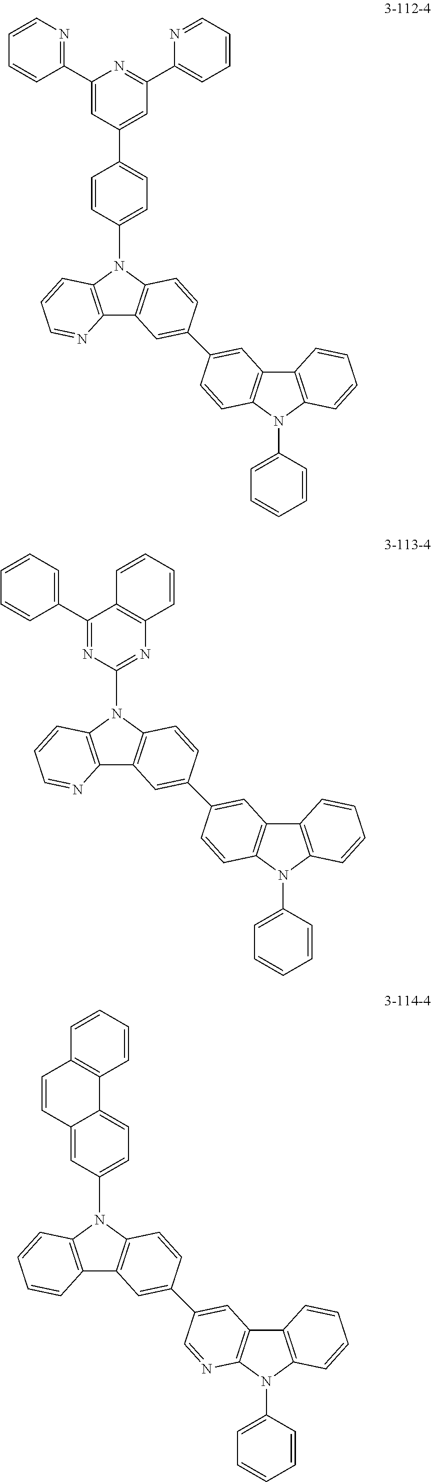 [SMILES: c1ccc(-n2c3ccccc3c3cc(-c4ccc5c(c4)c4ncccc4n5-c4ccc(-c5cc(-c6ccccn6)nc(-c6ccccn6)c5)cc4)ccc32)cc1 ; c1ccc(-c2nc(-n3c4ccc(-c5ccc6c(c5)c5ccccc5n6-c5ccccc5)cc4c4ncccc43)nc3ccccc23)cc1 ; c1ccc(-n2c3ccccc3c3cc(-c4ccc5c(c4)c4ccccc4n5-c4ccc5c(ccc6ccccc65)c4)cnc32)cc1]

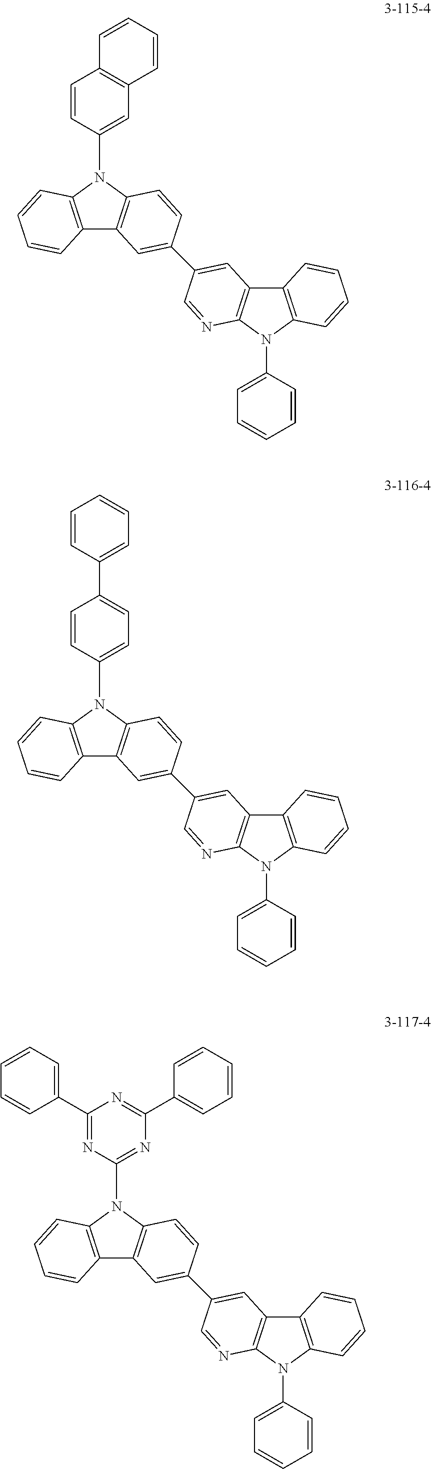 [SMILES: c1ccc(-n2c3ccccc3c3cc(-c4ccc5c(c4)c4ccccc4n5-c4ccc5ccccc5c4)cnc32)cc1 ; c1ccc(-c2ccc(-n3c4ccccc4c4cc(-c5cnc6c(c5)c5ccccc5n6-c5ccccc5)ccc43)cc2)cc1 ; c1ccc(-c2nc(-c3ccccc3)nc(-n3c4ccccc4c4cc(-c5cnc6c(c5)c5ccccc5n6-c5ccccc5)ccc43)n2)cc1]

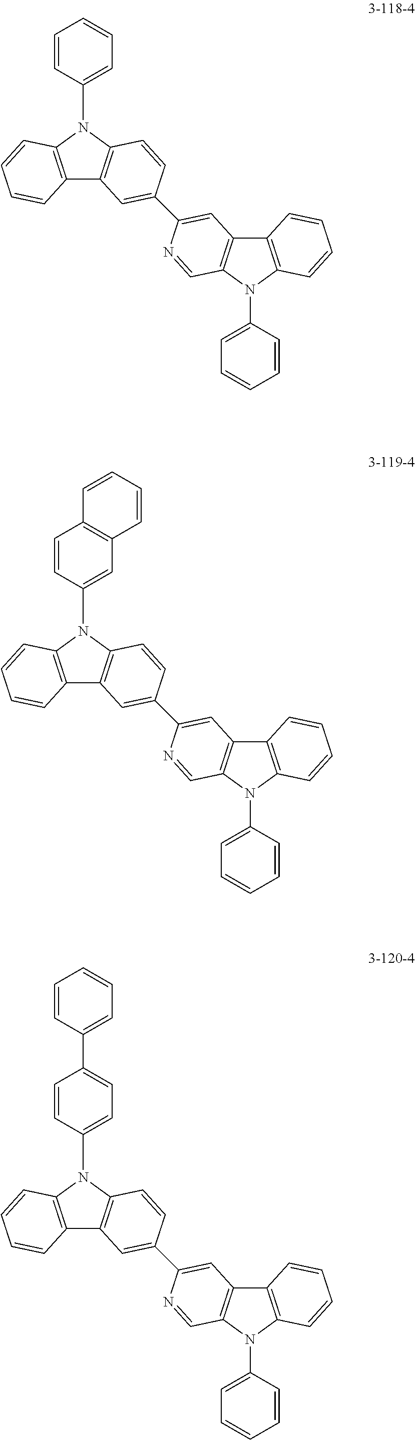 [SMILES: c1ccc(-n2c3ccccc3c3cc(-c4cc5c6ccccc6n(-c6ccccc6)c5cn4)ccc32)cc1 ; c1ccc(-n2c3ccccc3c3cc(-c4ccc5c(c4)c4ccccc4n5-c4ccc5ccccc5c4)ncc32)cc1 ; c1ccc(-c2ccc(-n3c4ccccc4c4cc(-c5cc6c7ccccc7n(-c7ccccc7)c6cn5)ccc43)cc2)cc1]

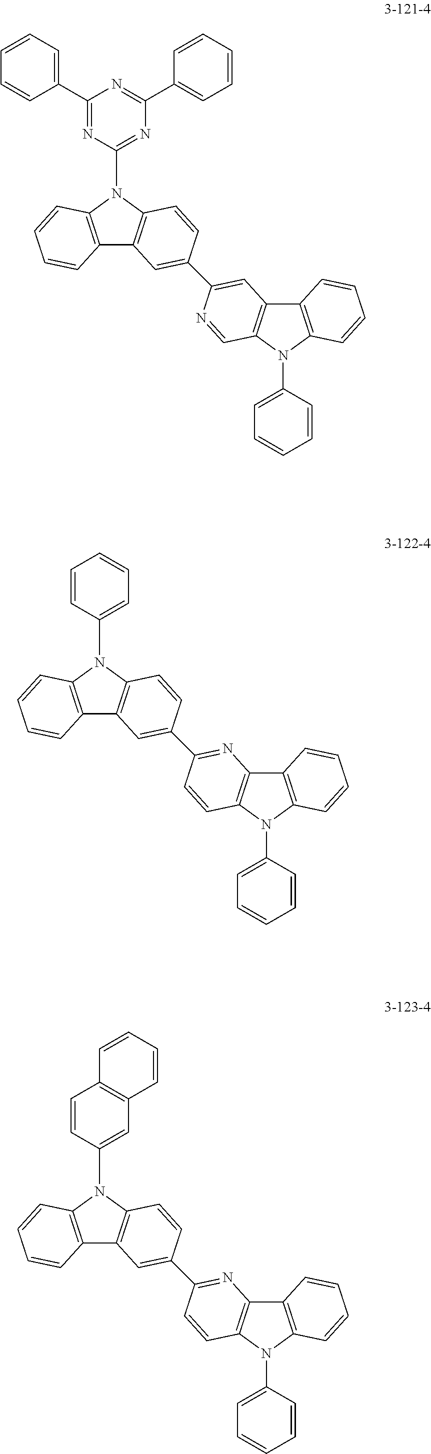 [SMILES: c1ccc(-c2nc(-c3ccccc3)nc(-n3c4ccccc4c4cc(-c5cc6c7ccccc7n(-c7ccccc7)c6cn5)ccc43)n2)cc1 ; c1ccc(-n2c3ccccc3c3cc(-c4ccc5c(n4)c4ccccc4n5-c4ccccc4)ccc32)cc1 ; c1ccc(-n2c3ccccc3c3nc(-c4ccc5c(c4)c4ccccc4n5-c4ccc5ccccc5c4)ccc32)cc1]

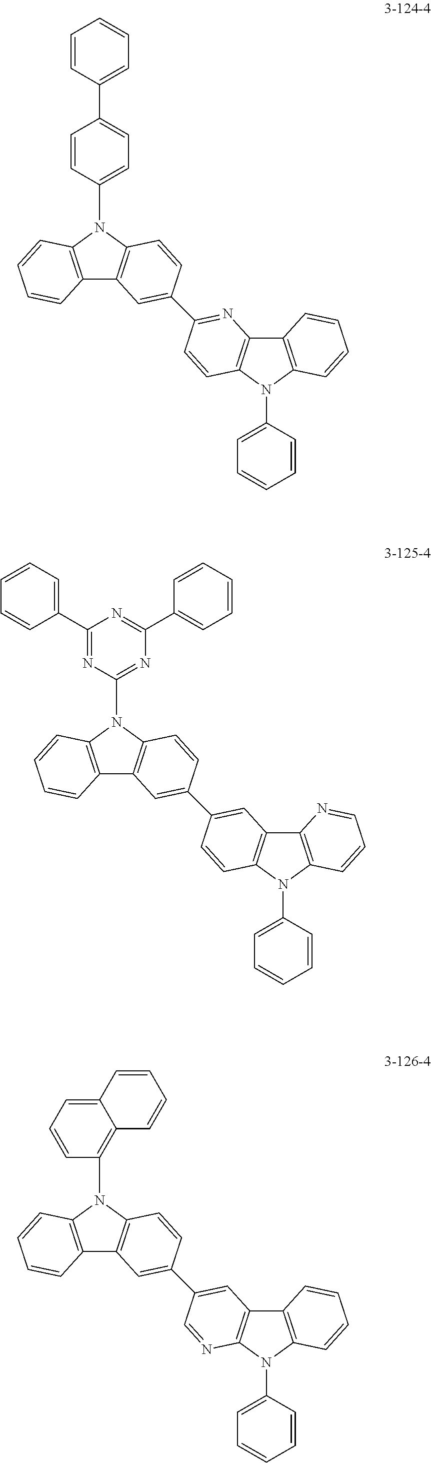 [SMILES: c1ccc(-c2ccc(-n3c4ccccc4c4cc(-c5ccc6c(n5)c5ccccc5n6-c5ccccc5)ccc43)cc2)cc1 ; c1ccc(-c2nc(-c3ccccc3)nc(-n3c4ccccc4c4cc(-c5ccc6c(c5)c5ncccc5n6-c5ccccc5)ccc43)n2)cc1 ; c1ccc(-n2c3ccccc3c3cc(-c4ccc5c(c4)c4ccccc4n5-c4cccc5ccccc45)cnc32)cc1]

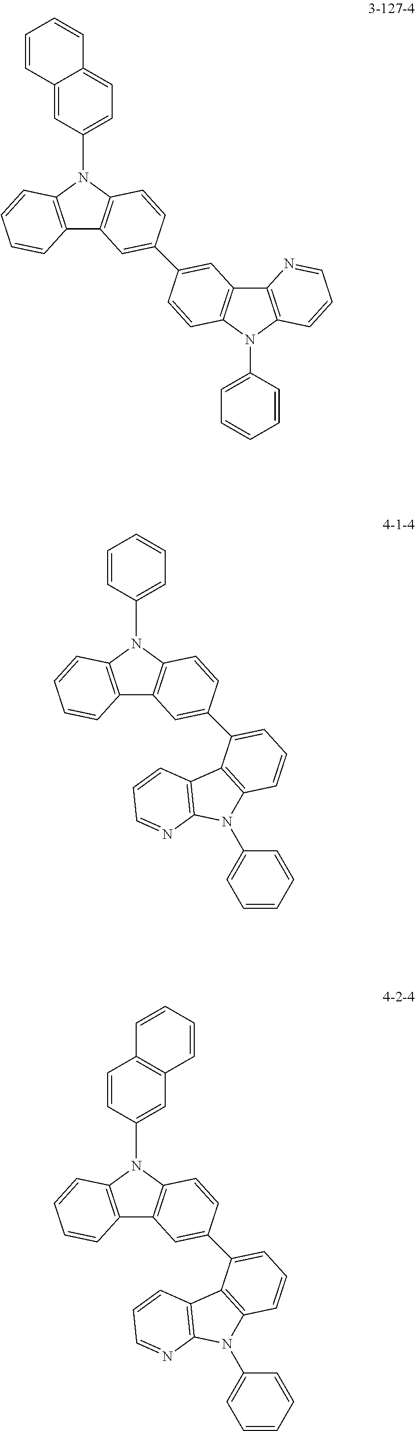 [SMILES: c1ccc(-n2c3ccc(-c4ccc5c(c4)c4ccccc4n5-c4ccc5ccccc5c4)cc3c3ncccc32)cc1 ; c1ccc(-n2c3ccccc3c3cc(-c4cccc5c4c4cccnc4n5-c4ccccc4)ccc32)cc1 ; c1ccc(-n2c3cccc(-c4ccc5c(c4)c4ccccc4n5-c4ccc5ccccc5c4)c3c3cccnc32)cc1]

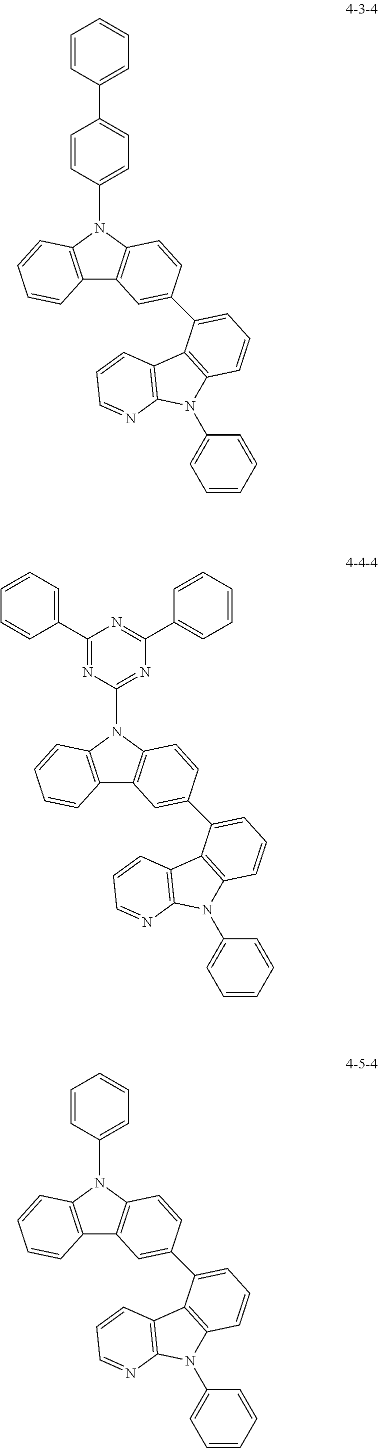 [SMILES: c1ccc(-c2ccc(-n3c4ccccc4c4cc(-c5cccc6c5c5cccnc5n6-c5ccccc5)ccc43)cc2)cc1 ; c1ccc(-c2nc(-c3ccccc3)nc(-n3c4ccccc4c4cc(-c5cccc6c5c5cccnc5n6-c5ccccc5)ccc43)n2)cc1 ; c1ccc(-n2c3ccccc3c3cc(-c4cccc5c4c4cccnc4n5-c4ccccc4)ccc32)cc1]

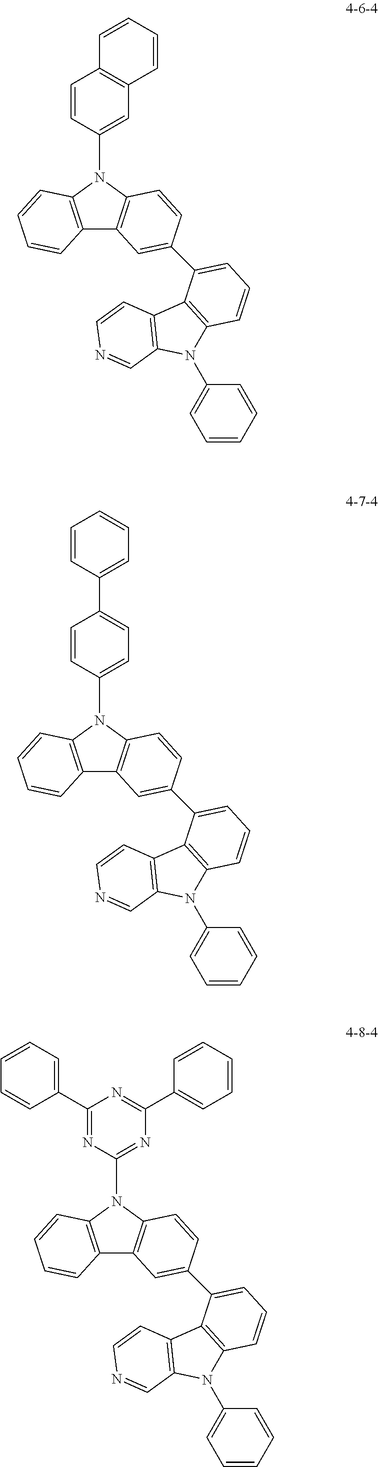 [SMILES: c1ccc(-n2c3cnccc3c3c(-c4ccc5c(c4)c4ccccc4n5-c4ccc5ccccc5c4)cccc32)cc1 ; c1ccc(-c2ccc(-n3c4ccccc4c4cc(-c5cccc6c5c5ccncc5n6-c5ccccc5)ccc43)cc2)cc1 ; c1ccc(-c2nc(-c3ccccc3)nc(-n3c4ccccc4c4cc(-c5cccc6c5c5ccncc5n6-c5ccccc5)ccc43)n2)cc1]

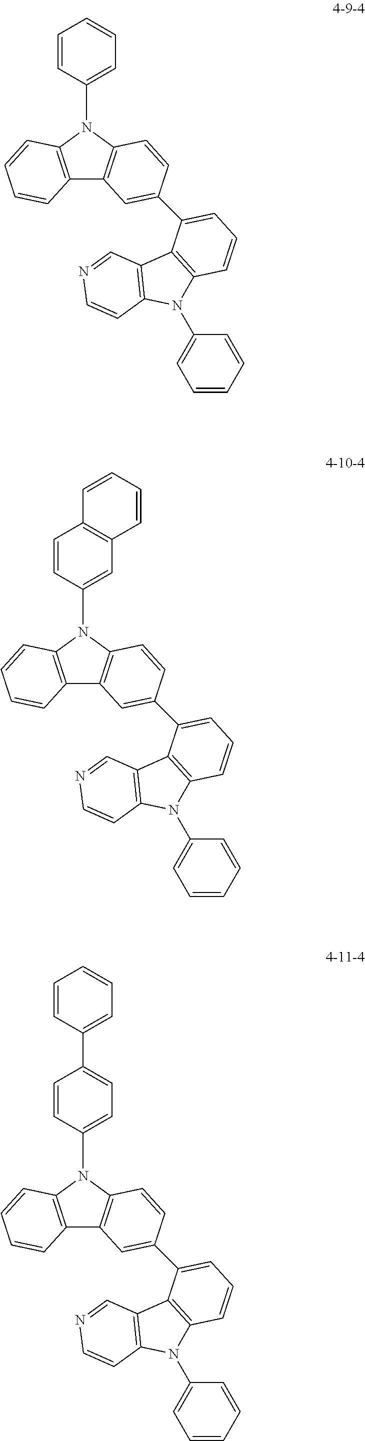 [SMILES: c1ccc(-n2c3ccccc3c3cc(-c4cccc5c4c4cnccc4n5-c4ccccc4)ccc32)cc1 ; c1ccc(-n2c3ccncc3c3c(-c4ccc5c(c4)c4ccccc4n5-c4ccc5ccccc5c4)cccc32)cc1 ; c1ccc(-c2ccc(-n3c4ccccc4c4cc(-c5cccc6c5c5cnccc5n6-c5ccccc5)ccc43)cc2)cc1]

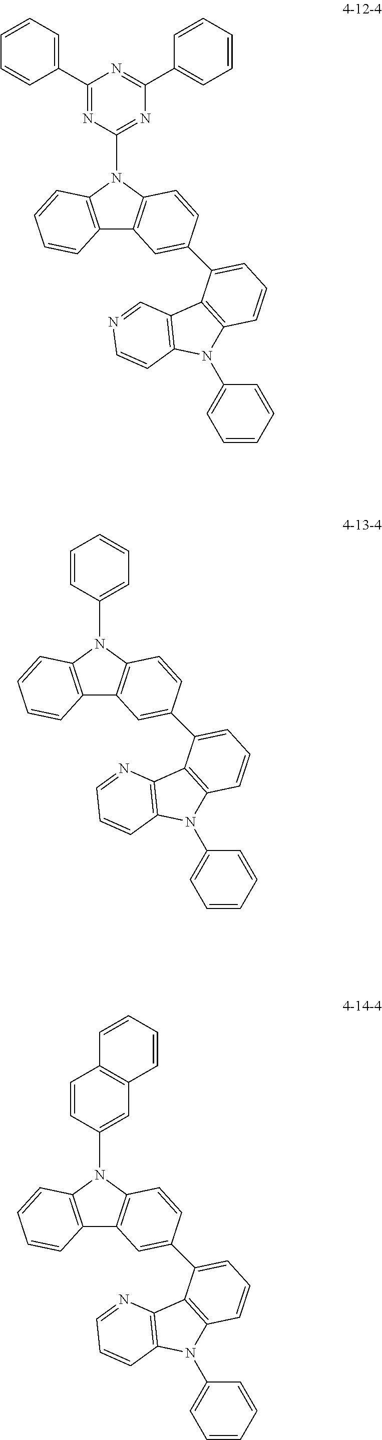 [SMILES: c1ccc(-c2nc(-c3ccccc3)nc(-n3c4ccccc4c4cc(-c5cccc6c5c5cnccc5n6-c5ccccc5)ccc43)n2)cc1 ; c1ccc(-n2c3ccccc3c3cc(-c4cccc5c4c4ncccc4n5-c4ccccc4)ccc32)cc1 ; c1ccc(-n2c3cccnc3c3c(-c4ccc5c(c4)c4ccccc4n5-c4ccc5ccccc5c4)cccc32)cc1]

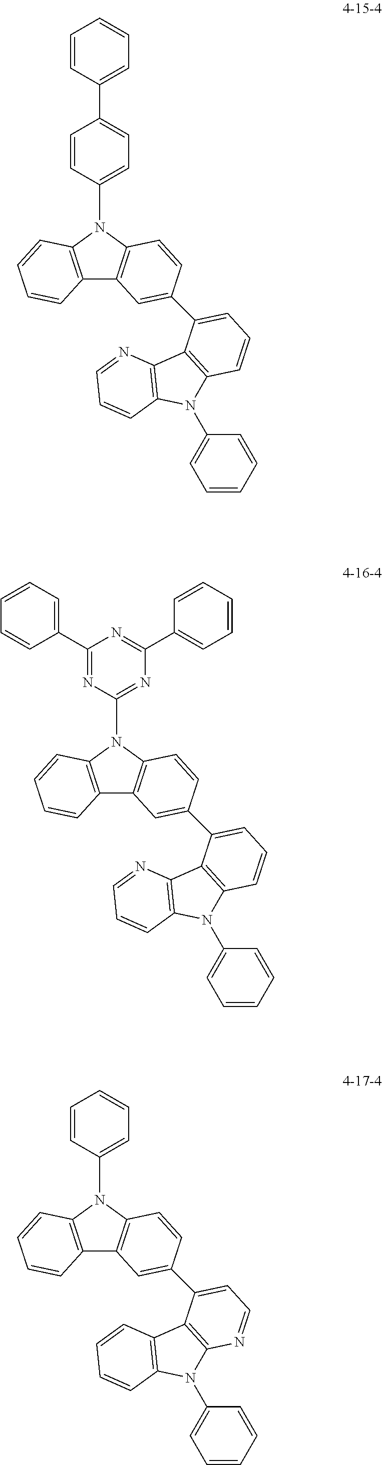 [SMILES: c1ccc(-c2ccc(-n3c4ccccc4c4cc(-c5cccc6c5c5ncccc5n6-c5ccccc5)ccc43)cc2)cc1 ; c1ccc(-c2nc(-c3ccccc3)nc(-n3c4ccccc4c4cc(-c5cccc6c5c5ncccc5n6-c5ccccc5)ccc43)n2)cc1 ; c1ccc(-n2c3ccccc3c3cc(-c4ccnc5c4c4ccccc4n5-c4ccccc4)ccc32)cc1]

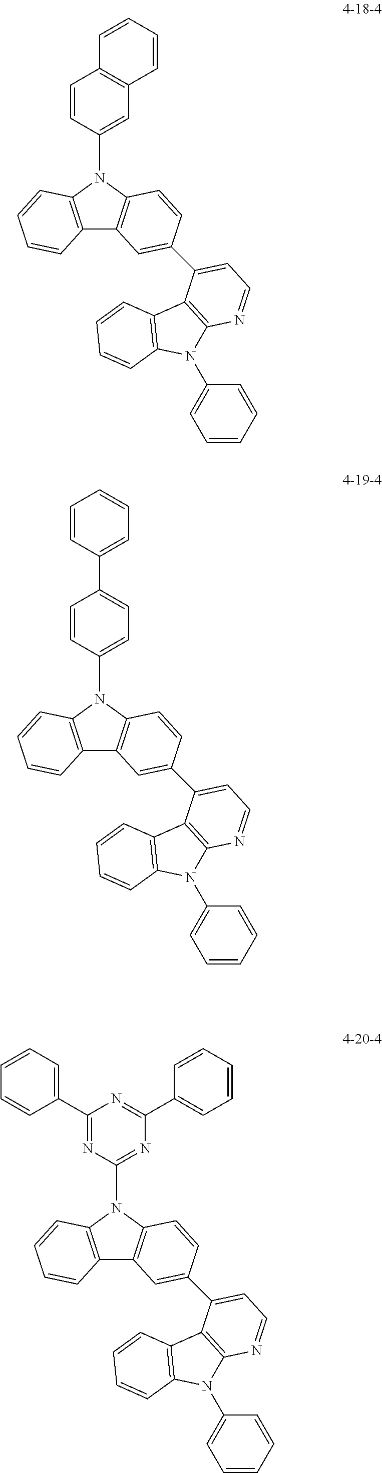 [SMILES: c1ccc(-n2c3ccccc3c3c(-c4ccc5c(c4)c4ccccc4n5-c4ccc5ccccc5c4)ccnc32)cc1 ; c1ccc(-c2ccc(-n3c4ccccc4c4cc(-c5ccnc6c5c5ccccc5n6-c5ccccc5)ccc43)cc2)cc1 ; c1ccc(-c2nc(-c3ccccc3)nc(-n3c4ccccc4c4cc(-c5ccnc6c5c5ccccc5n6-c5ccccc5)ccc43)n2)cc1]

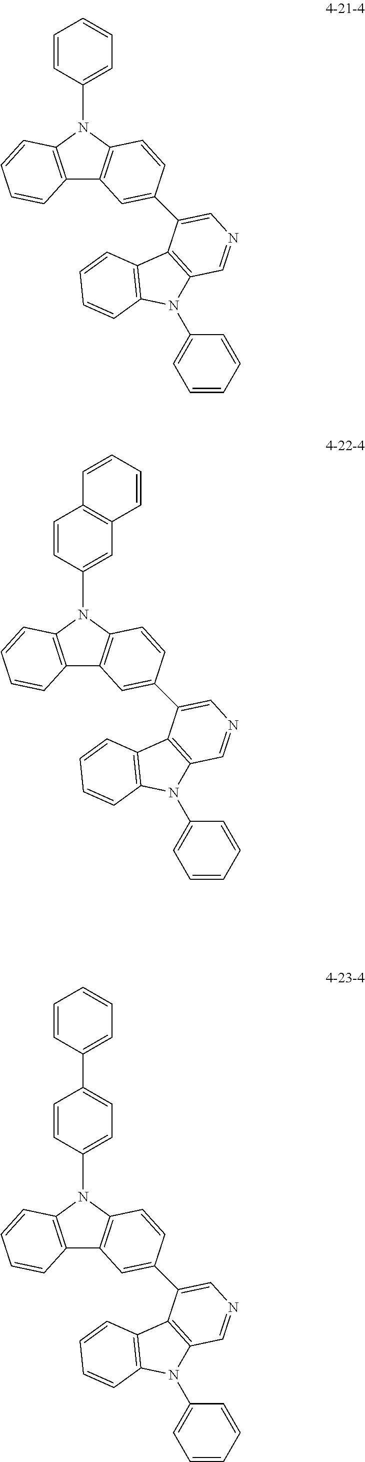 [SMILES: c1ccc(-n2c3ccccc3c3cc(-c4cncc5c4c4ccccc4n5-c4ccccc4)ccc32)cc1 ; c1ccc(-n2c3ccccc3c3c(-c4ccc5c(c4)c4ccccc4n5-c4ccc5ccccc5c4)cncc32)cc1 ; c1ccc(-c2ccc(-n3c4ccccc4c4cc(-c5cncc6c5c5ccccc5n6-c5ccccc5)ccc43)cc2)cc1]

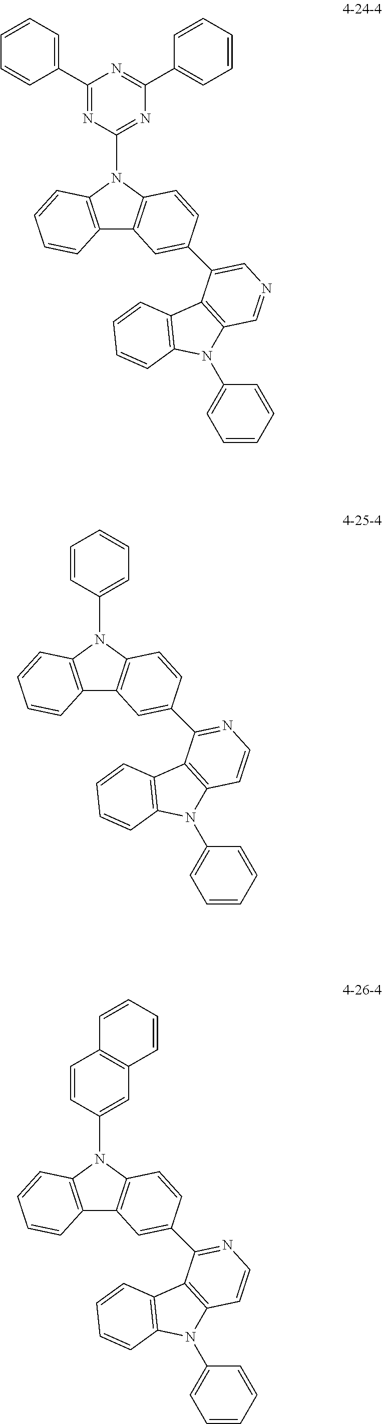 [SMILES: c1ccc(-c2nc(-c3ccccc3)nc(-n3c4ccccc4c4cc(-c5cncc6c5c5ccccc5n6-c5ccccc5)ccc43)n2)cc1 ; c1ccc(-n2c3ccccc3c3cc(-c4nccc5c4c4ccccc4n5-c4ccccc4)ccc32)cc1 ; c1ccc(-n2c3ccccc3c3c(-c4ccc5c(c4)c4ccccc4n5-c4ccc5ccccc5c4)nccc32)cc1]

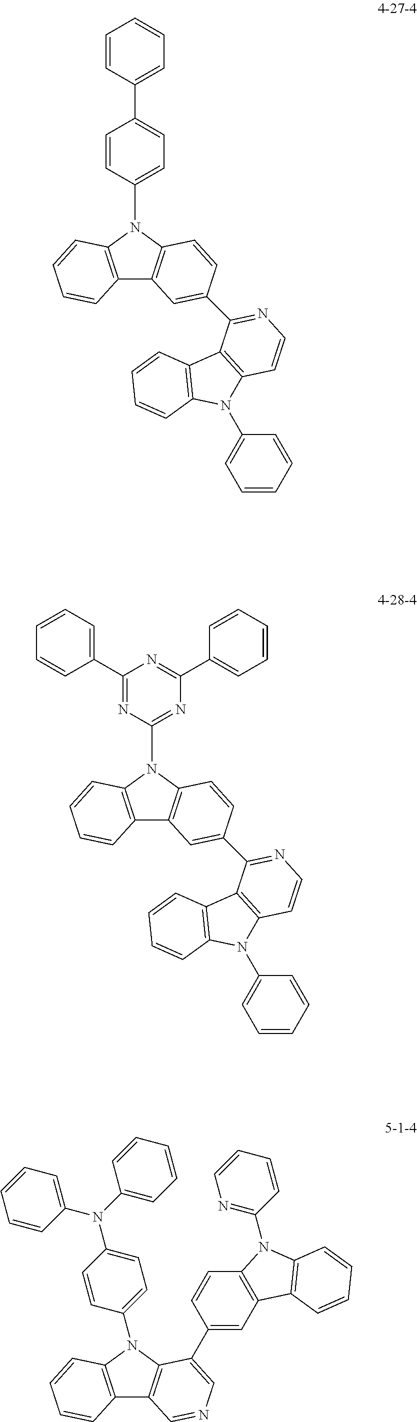 [SMILES: c1ccc(-c2ccc(-n3c4ccccc4c4cc(-c5nccc6c5c5ccccc5n6-c5ccccc5)ccc43)cc2)cc1 ; c1ccc(-c2nc(-c3ccccc3)nc(-n3c4ccccc4c4cc(-c5nccc6c5c5ccccc5n6-c5ccccc5)ccc43)n2)cc1 ; c1ccc(N(c2ccccc2)c2ccc(-n3c4ccccc4c4cncc(-c5ccc6c(c5)c5ccccc5n6-c5ccccn5)c43)cc2)cc1]

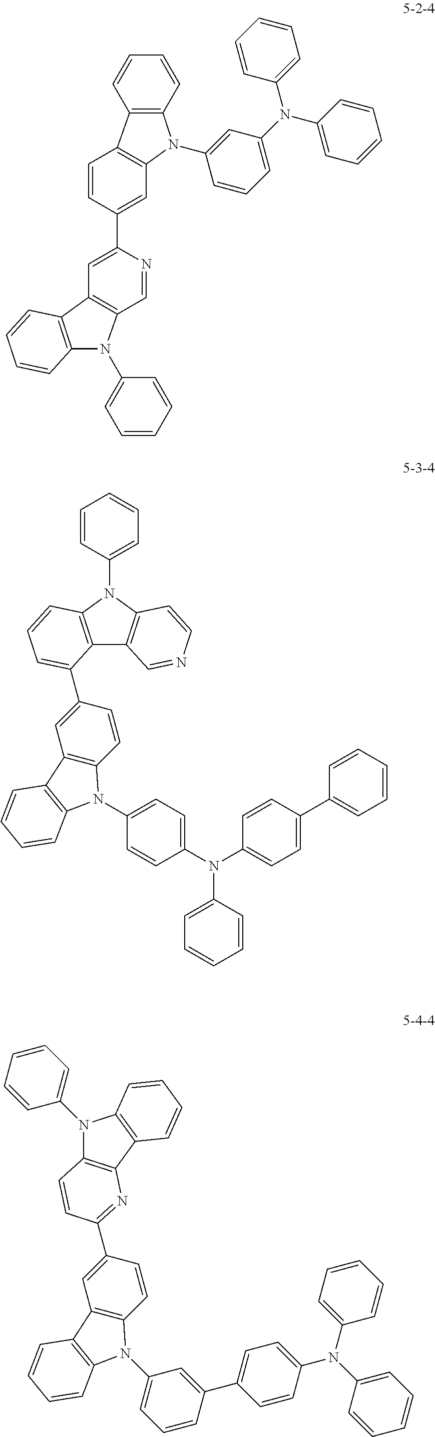 [SMILES: c1ccc(N(c2ccccc2)c2cccc(-n3c4ccccc4c4ccc(-c5cc6c7ccccc7n(-c7ccccc7)c6cn5)cc43)c2)cc1 ; c1ccc(-c2ccc(N(c3ccccc3)c3ccc(-n4c5ccccc5c5cc(-c6cccc7c6c6cnccc6n7-c6ccccc6)ccc54)cc3)cc2)cc1 ; c1ccc(N(c2ccccc2)c2ccc(-c3cccc(-n4c5ccccc5c5cc(-c6ccc7c(n6)c6ccccc6n7-c6ccccc6)ccc54)c3)cc2)cc1]

In another embodiment, the present invention provides a compound for an organic electronic element, represented by Formula 4-1.

In still another embodiment, the present invention provides an organic electronic element containing the compound represented by Formula 4-1.

Here, the organic electronic element may include: a first electrode; a second electrode; and an organic material layer positioned between the first electrode and the second electrode, wherein the organic material layer may contain a compound represented by Formula 4-1, and the compound represented by Formula 4-1 may be contained in at least one of a hole injection layer, a hole transport layer, an auxiliary light emitting layer, a light emitting layer, an electron transport layer, and an electron injection layer for an organic material layer. Especially, the compound represented by Formula 4-1 may be contained in the light emitting layer.

That is, the compound represented by Formula 4-1 may be used as a material for a hole injection layer, a hole transport layer, an auxiliary light emitting layer, a light emitting layer, an electron transport layer, or an electron injection layer. Especially, the compound represented by Formula 4-1 may be used as a material for the light emitting layer. The present invention provides, specifically, an organic electronic element including the organic material layer containing one of the compounds represented by Formulas 4-2 to 4-13, and more specifically, an organic electronic element including the organic material layer containing the compound represented by an individual formula (1-1-4 to 1-28-4, 2-1-4 to 2-128-4, 3-1-4 to 3-127-4, 4-1-4 to 4-28-4, and 5-1-4 to 5-4-4).

In still another embodiment, the present invention provides an organic electronic element, in which the compound is contained alone, two or more different types of the compounds are contained as a combination, or the compound is contained together with other compounds as a combination of two or more in at least one of the hole injection layer, the hole transport layer, the auxiliary light emitting layer, the light emitting layer, the electron transport layer, and the electron injection layer of the organic material layer. In other words, the compounds corresponding to Formulas 4-1 to 4-13 may be contained alone, a mixture of two or more kinds of compounds of Formulas 4-1 to 4-13 may be contained, or a mixture of the compound of claims and a compound not corresponding to the present invention may be contained in each of the layers. Here, the compounds that do not correspond to the present invention may be a single compound or two or more kinds of compounds. Here, when the compound is contained together with other compounds as a combination of two or more kinds of compounds, another compound may be a compound that is already known for each organic material layer, or a compound to be developed in the future. Here, the compounds contained in the organic material layer may be composed of only the same kind of compounds, or a mixture of two or more kinds of different compounds represented by formula 4-1.

In still another embodiment of the present invention, the present invention provides an organic electronic element further including a light efficiency improvement layer, which is formed on at least one of one side of one surface of the first electrode, which is opposite to the organic material layer and one side of one surface of the second electrode, which is opposite to the organic material layer.

Hereinafter, synthesis examples of the compound represented by Formula 4-1 and manufacturing examples of the organic electronic element according to the present invention will be described in detail by way of example. However, the following examples are only for illustrative purposes and are not intended to limit the scope of the invention.

### SYNTHESIS EXAMPLES

The product represented by Formula 4-1 according to the present invention is prepared by reaction of Sub 1-4 and Sub 2-4 as in Reaction Scheme 4-1 below, but are not limited thereto.

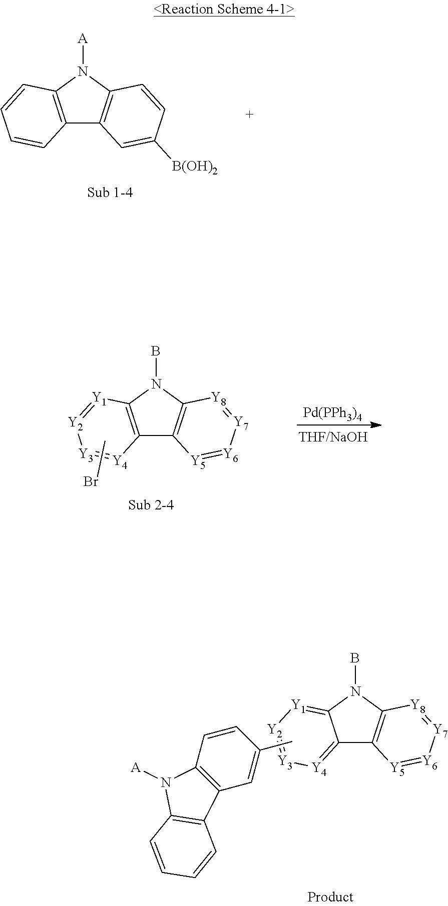

### I. Synthesis Example of Sub 1-4

Sub 1-4 in Reaction Scheme 4-1 may be synthesized via the reaction pathway of Reaction Scheme 4-2 below, but is not limited thereto.

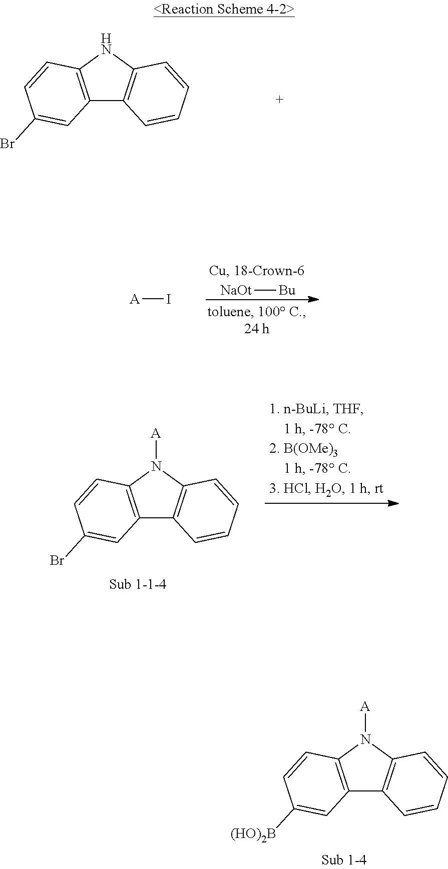

### Synthesis of Sub 1-1(1)-4

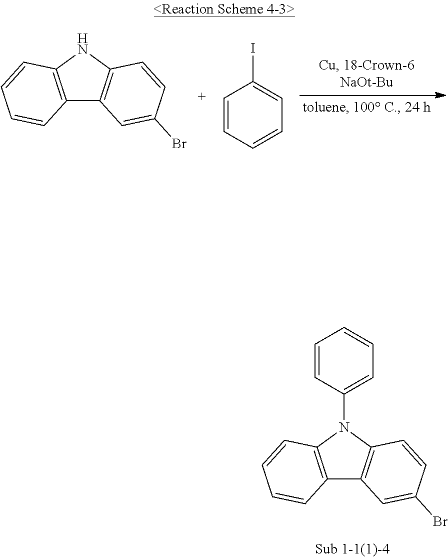

After bromo-9H-carbazole (50.0 g, 203 mmol) and iodobenzene (49 g, 240 mmol) were mixed with 800 mL of toluene, Cu (764 mg, 12 mmol), 18-Crown-6 (6.3 g, 24 mmol), and NaOt-Bu (57.6 g, 600 mmol) were added thereto, and the mixture was stirred under reflux at 1000 for 24 h. After extraction with ether and water, the organic layer was dried over MgSO₄ and concentrated, and then the generated organic material was subjected to silica gel column chromatography and recrystallization to give 36.6 g of Sub 1-1(1)-4 (yield: 57%).

Examples of Sub 1-1-4 are as follows, but are limited thereto, and FD-MS values thereof are shown in table 4-1

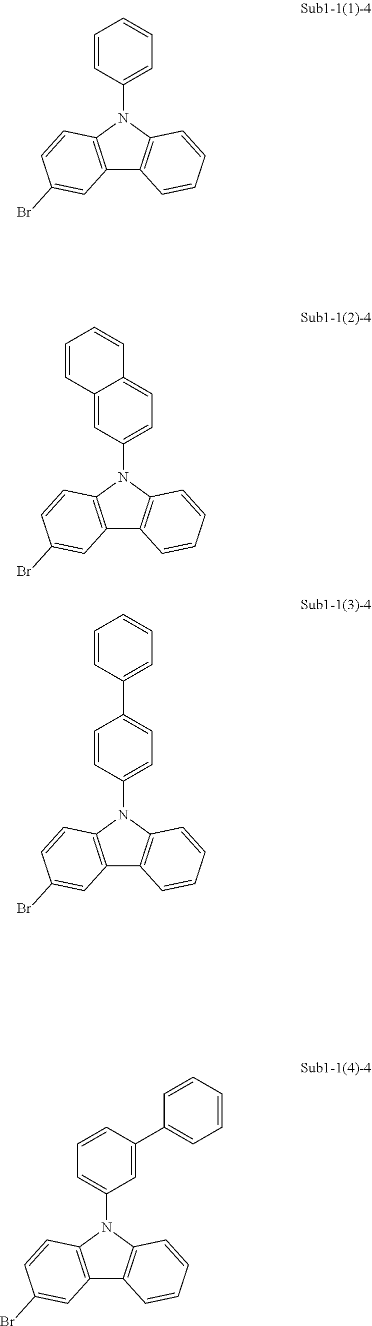 [SMILES: Brc1ccc2c(c1)c1ccccc1n2-c1ccccc1 ; Brc1ccc2c(c1)c1ccccc1n2-c1ccc2ccccc2c1 ; Brc1ccc2c(c1)c1ccccc1n2-c1ccc(-c2ccccc2)cc1 ; Brc1ccc2c(c1)c1ccccc1n2-c1cccc(-c2ccccc2)c1]

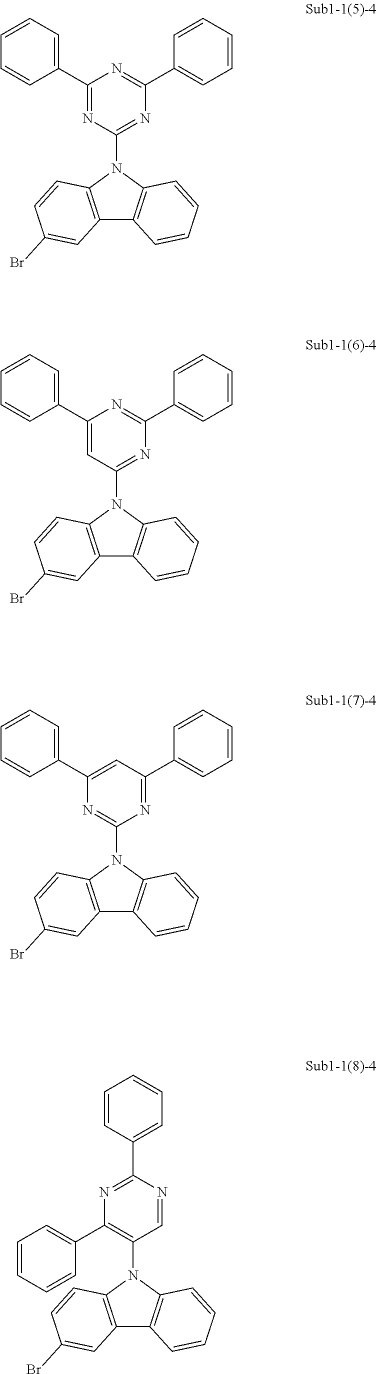 [SMILES: Brc1ccc2c(c1)c1ccccc1n2-c1nc(-c2ccccc2)nc(-c2ccccc2)n1 ; Brc1ccc2c(c1)c1ccccc1n2-c1cc(-c2ccccc2)nc(-c2ccccc2)n1 ; Brc1ccc2c(c1)c1ccccc1n2-c1nc(-c2ccccc2)cc(-c2ccccc2)n1 ; Brc1ccc2c(c1)c1ccccc1n2-c1cnc(-c2ccccc2)nc1-c1ccccc1]

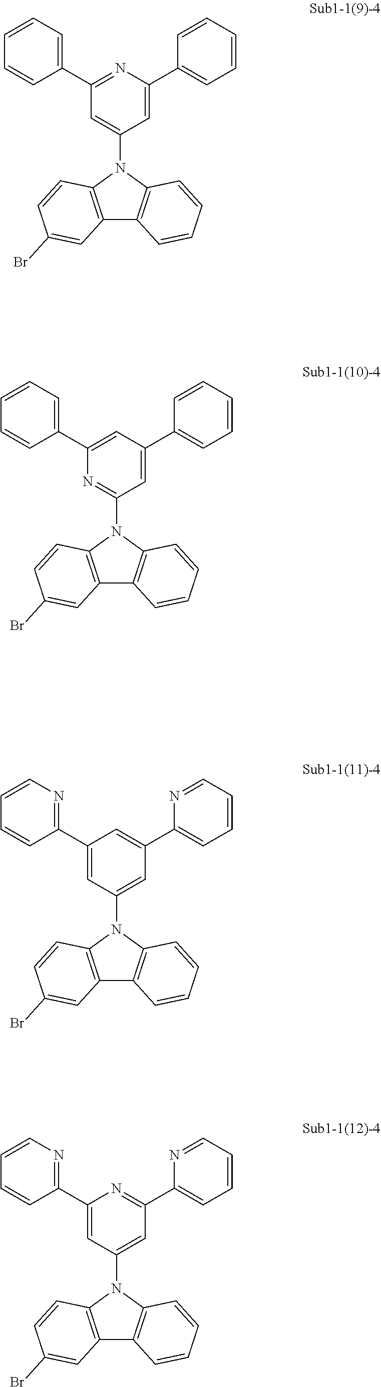 [SMILES: Brc1ccc2c(c1)c1ccccc1n2-c1cc(-c2ccccc2)nc(-c2ccccc2)c1 ; Brc1ccc2c(c1)c1ccccc1n2-c1cc(-c2ccccc2)cc(-c2ccccc2)n1 ; Brc1ccc2c(c1)c1ccccc1n2-c1cc(-c2ccccn2)cc(-c2ccccn2)c1 ; Brc1ccc2c(c1)c1ccccc1n2-c1cc(-c2ccccn2)nc(-c2ccccn2)c1]

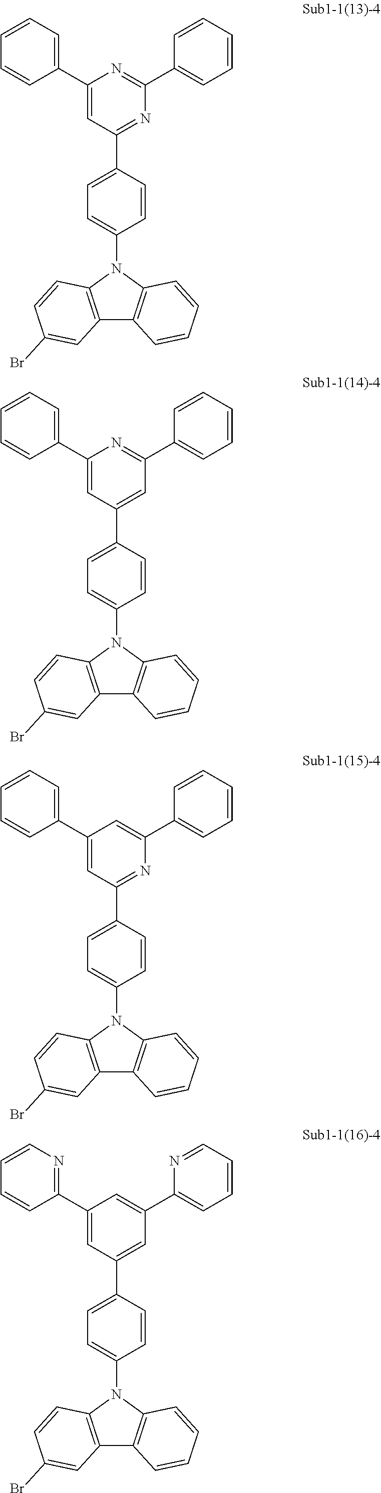

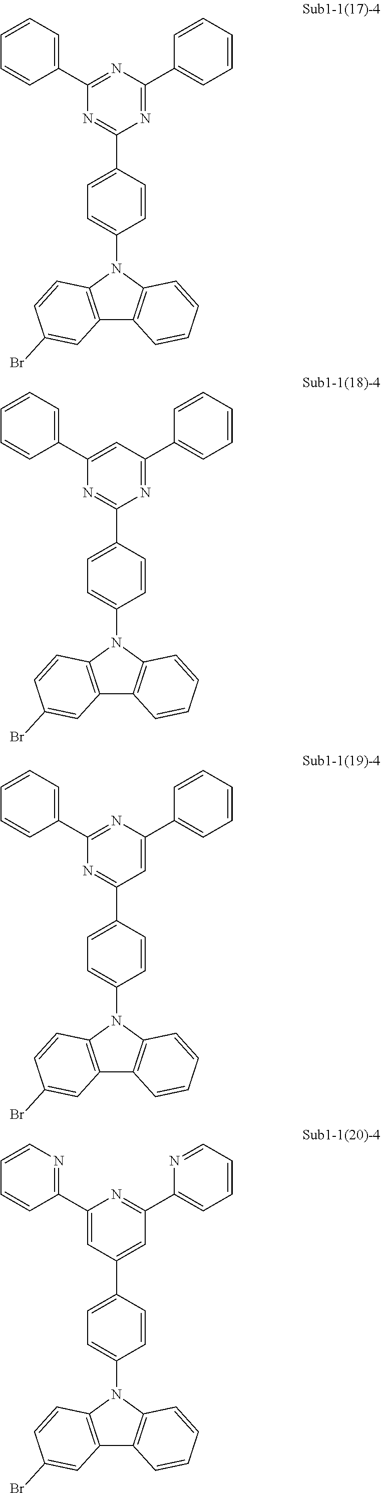

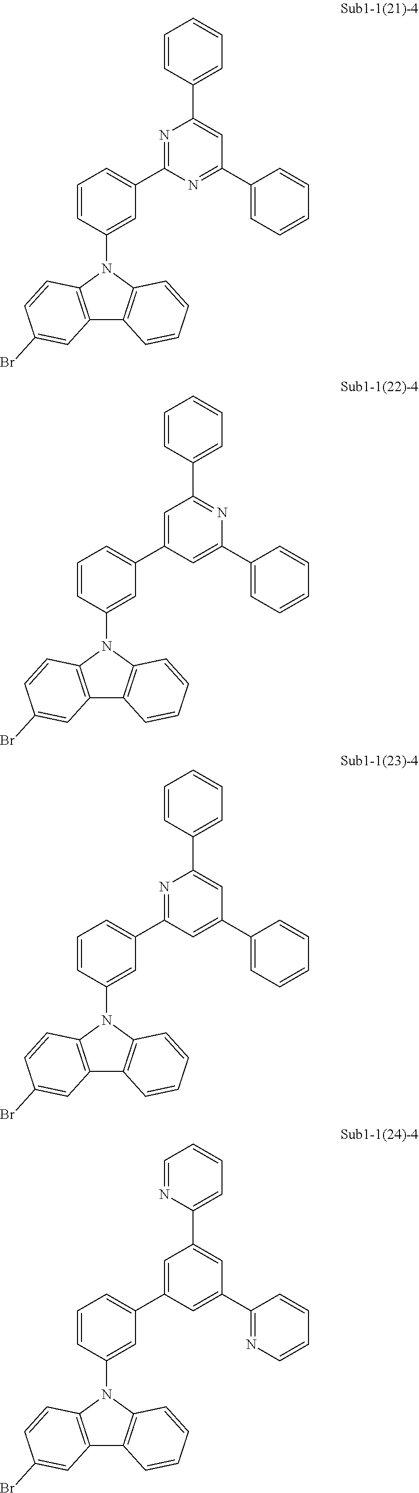 [SMILES: Brc1ccc2c(c1)c1ccccc1n2-c1cccc(-c2nc(-c3ccccc3)cc(-c3ccccc3)n2)c1 ; Brc1ccc2c(c1)c1ccccc1n2-c1cccc(-c2cc(-c3ccccc3)nc(-c3ccccc3)c2)c1 ; Brc1ccc2c(c1)c1ccccc1n2-c1cccc(-c2cc(-c3ccccc3)cc(-c3ccccc3)n2)c1 ; Brc1ccc2c(c1)c1ccccc1n2-c1cccc(-c2cc(-c3ccccn3)cc(-c3ccccn3)c2)c1]

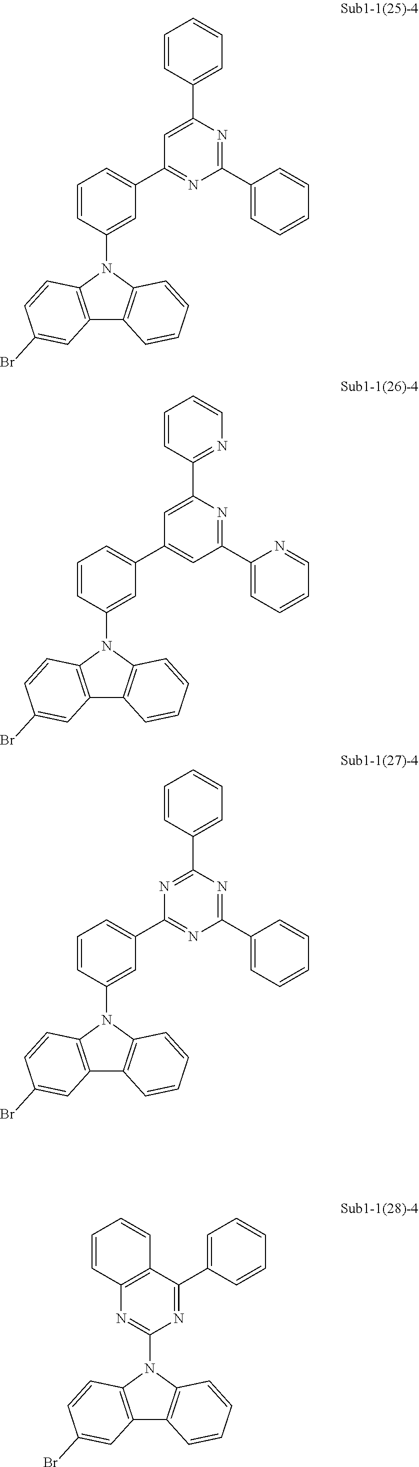 [SMILES: Brc1ccc2c(c1)c1ccccc1n2-c1cccc(-c2cc(-c3ccccc3)nc(-c3ccccc3)n2)c1 ; Brc1ccc2c(c1)c1ccccc1n2-c1cccc(-c2cc(-c3ccccn3)nc(-c3ccccn3)c2)c1 ; Brc1ccc2c(c1)c1ccccc1n2-c1cccc(-c2nc(-c3ccccc3)nc(-c3ccccc3)n2)c1 ; Brc1ccc2c(c1)c1ccccc1n2-c1nc(-c2ccccc2)c2ccccc2n1]

TABLE 4-1

| Compound | FD-MS | Compound | FD-MS |
|---|---|---|---|
| Sub1-1(1)-4 | m/z = 321.02(C₁₈H₁₂BrN = 322.20) | Sub1-1(2)-4 | m/z = 371.03(C₂₂H₁₄BrN = 372.26) |
| Sub1-1(3)-4 | m/z = 397.05(C₂₄H₁₆BrN = 398.29) | Sub1-1(4)-4 | m/z = 397.05(C₂₄H₁₆BrN = 398.29) |
| Sub1-1(5)-4 | m/z = 476.06(C₂₂H₁₇BrN₄ = 477.35) | Sub1-1(6)-4 | m/z = 475.07(C₂₈H₁₈BrN₃ = 476.37) |
| Sub1-1(7)-4 | m/z = 475.07(C₂₈H₁₈BrN₃ = 476.37) | Sub1-1(8)-4 | m/z = 475.07(C₂₈H₁₈BrN₃ = 476.37) |
| Sub1-1(9)-4 | m/z = 474.07(C₂₉H₁₉BrN₂ = 475.38) | Sub1-1(10)-4 | m/z = 474.07(C₂₉H₁₉BrN₂ = 475.38) |
| Sub1-1(11)-4 | m/z = 475.07(C₂₈H₁₉BrN₃ = 476.37 | Sub1-1(12)-4 | m/z = 476.06(C₂₇H₁₇BrN₄ = 477.35) |
| Sub1-1(13)-4 | m/z = 551.10(C₃₄H₂₂BrN₃ = 552.46) | Sub1-1(14)-4 | m/z = 550.10(C₃₅H₂₃BrN₂ = 551.47) |
| Sub1-1(15)-4 | m/z = 550.10(C₃₅H₂₃BrN₂ = 551.47 | Sub1-1(16)-4 | m/z = 551.10(C₃₄H₂₂BrN₃ = 552.46) |
| Sub1-1(17)-4 | m/z = 552.09(C₃₂H₂₁BrN₄ = 553.45) | Sub1-1(18)-4 | m/z = 551.10(C₃₄H₂₂BrN₃ = 552.46) |
| Sub1-1(19)-4 | m/z = 551.10(C₃₄H₂₂BrN₃ = 552.46) | Sub1-1(20)-4 | m/z = 552.09(C₃₃H₂₁BrN₄ = 553.45) |
| Sub1-1(21)-4 | m/z = 551.10(C₃₄H₂₂BrN₃ = 552.46) | Sub1-1(22)-4 | m/z = 550.10(C₃₅H₂₃BrN₂ = 551.47) |
| Sub1-1(23)-4 | m/z = 550.10(C₃₅H₂₃BrN₂ = 551.47) | Sub1-1(24)-4 | m/z = 551.10(C₃₄H₂₂BrN₃ = 552.46) |
| Sub1-1(25)-4 | m/z = 551.10(C₃₄H₂₂BrN₃ = 552.46 | Sub1-1(26)-4 | m/z = 552.09(C₃₃H₂₁BrN₄ = 553.45) |
| Sub1-1(27)-4 | m/z = 552.09(C₃₃H₂₁BrN₄ = 553.45) | Sub1-1(28)-4 | m/z = 449.05(C₂₆H₁₆BrN₃ = 450.33) |

### Synthesis of Sub 1(1)-4

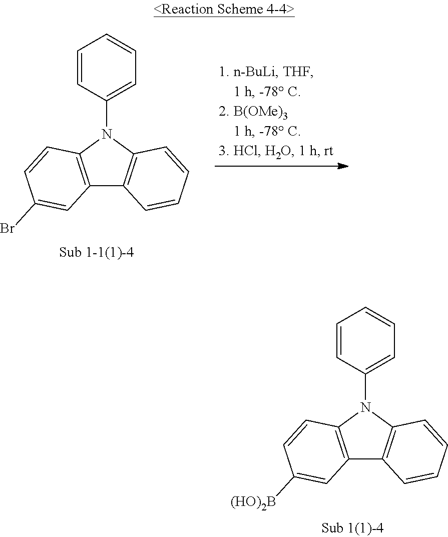 [SMILES: Brc1ccc2c(c1)c1ccccc1n2-c1ccccc1 ; OB(O)c1ccc2c(c1)c1ccccc1n2-c1ccccc1]

A two-necked RBF was equipped with a dropping-funnel, and Sub 1(1)-4 (38 g, 118 mmol) was dissolved in 500 ml of THF and the temperature was maintained at −78□. After stirring for 1 h, trimethoxyborate (18.4 g, 177 mmol) was slowly added dropwise, followed by again stirring for 1 h. Upon the completion of the reaction, 500 ml of 5% hydrochloric acid was added, followed by stirring at room temperature for 1 h, extraction with water and ethyl acetate, concentration, and recrystallization with MC and Hexane, thereby obtaining 20.3 g of compound Sub 1(1)-4 (yield: 60%).

Examples of Sub 1-4 are as follows, but are limited thereto, and FD-MS values thereof are shown in table 4-2

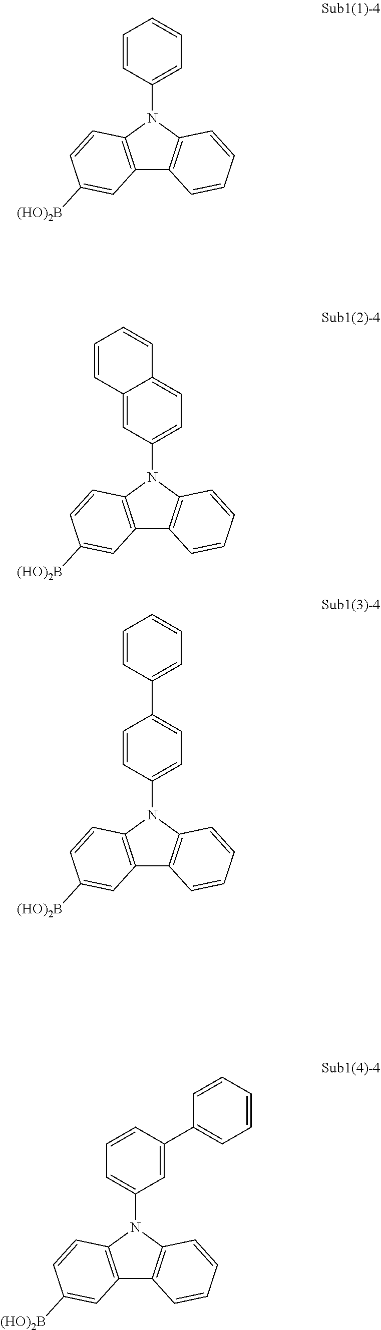 [SMILES: OB(O)c1ccc2c(c1)c1ccccc1n2-c1ccccc1 ; OB(O)c1ccc2c(c1)c1ccccc1n2-c1ccc2ccccc2c1 ; OB(O)c1ccc2c(c1)c1ccccc1n2-c1ccc(-c2ccccc2)cc1 ; OB(O)c1ccc2c(c1)c1ccccc1n2-c1cccc(-c2ccccc2)c1]

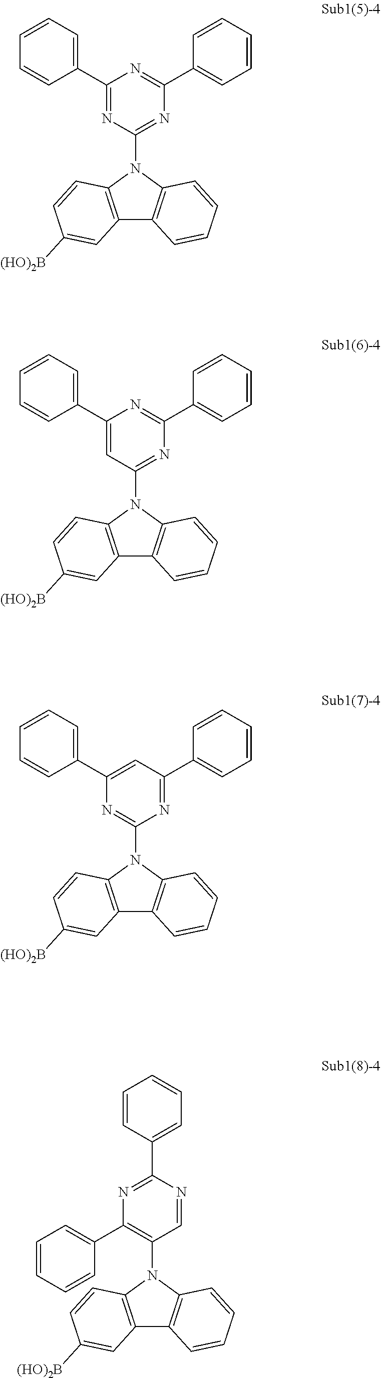 [SMILES: OB(O)c1ccc2c(c1)c1ccccc1n2-c1nc(-c2ccccc2)nc(-c2ccccc2)n1 ; OB(O)c1ccc2c(c1)c1ccccc1n2-c1cc(-c2ccccc2)nc(-c2ccccc2)n1 ; OB(O)c1ccc2c(c1)c1ccccc1n2-c1nc(-c2ccccc2)cc(-c2ccccc2)n1 ; OB(O)c1ccc2c(c1)c1ccccc1n2-c1cnc(-c2ccccc2)nc1-c1ccccc1]

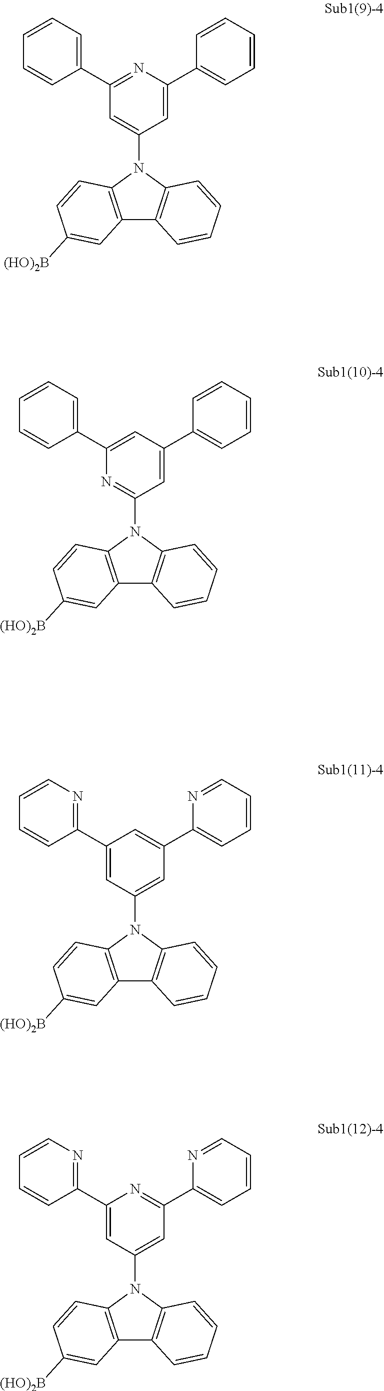 [SMILES: OB(O)c1ccc2c(c1)c1ccccc1n2-c1cc(-c2ccccc2)nc(-c2ccccc2)c1 ; OB(O)c1ccc2c(c1)c1ccccc1n2-c1cc(-c2ccccc2)cc(-c2ccccc2)n1 ; OB(O)c1ccc2c(c1)c1ccccc1n2-c1cc(-c2ccccn2)cc(-c2ccccn2)c1 ; OB(O)c1ccc2c(c1)c1ccccc1n2-c1cc(-c2ccccn2)nc(-c2ccccn2)c1]

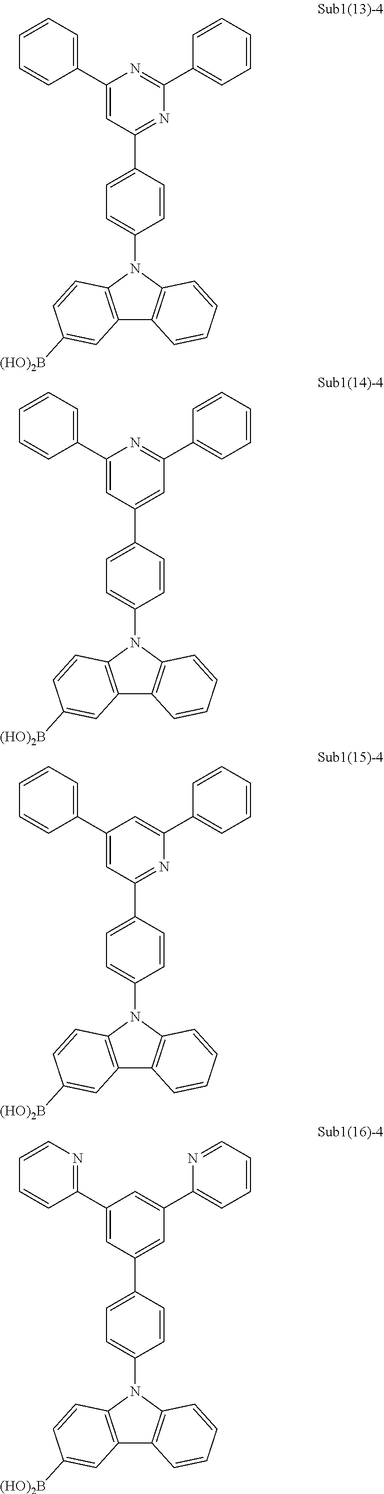

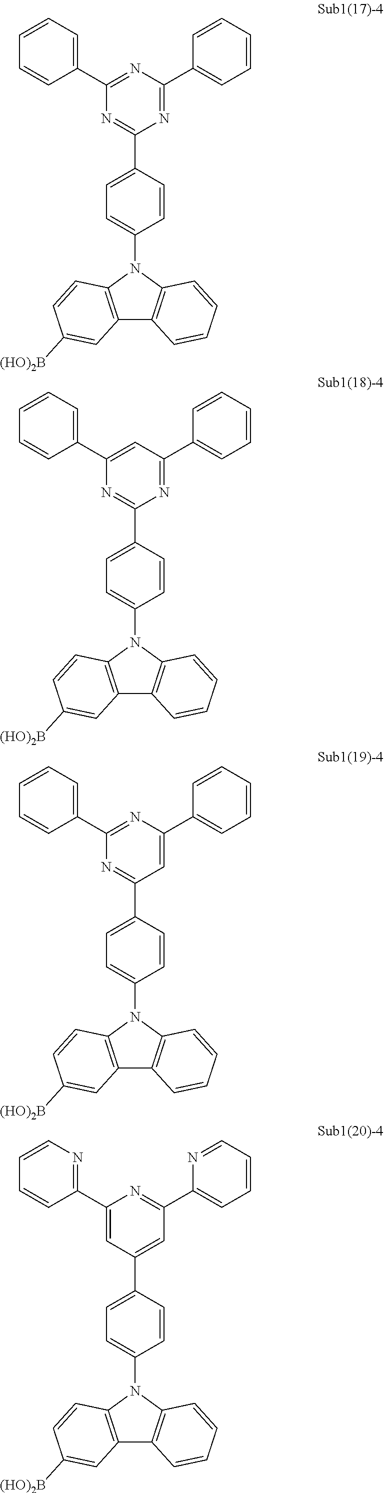

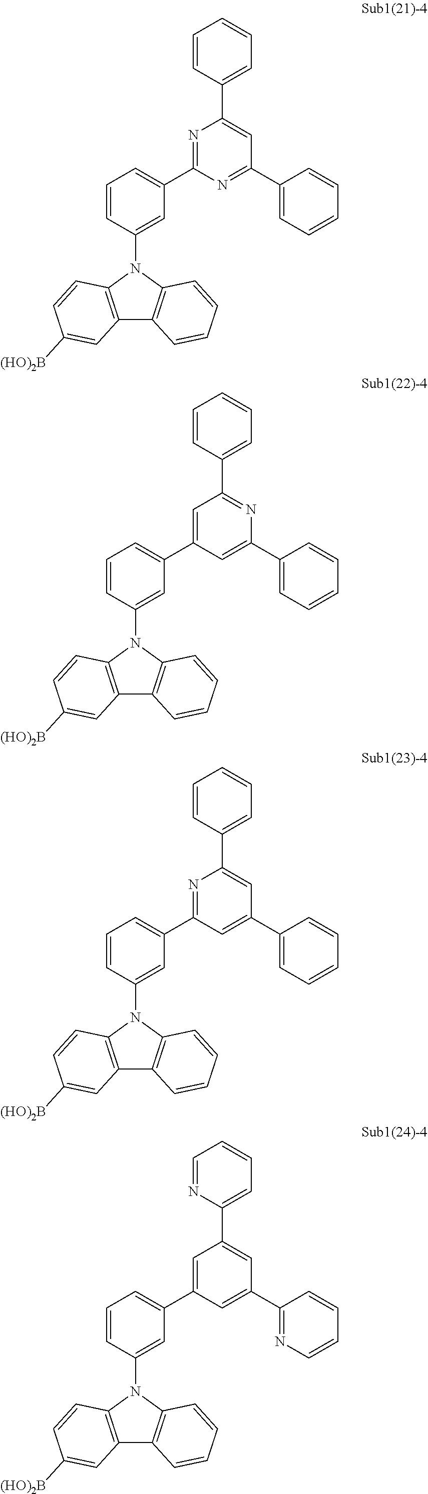 [SMILES: OB(O)c1ccc2c(c1)c1ccccc1n2-c1cccc(-c2nc(-c3ccccc3)cc(-c3ccccc3)n2)c1 ; OB(O)c1ccc2c(c1)c1ccccc1n2-c1cccc(-c2cc(-c3ccccc3)nc(-c3ccccc3)c2)c1 ; OB(O)c1ccc2c(c1)c1ccccc1n2-c1cccc(-c2cc(-c3ccccc3)cc(-c3ccccc3)n2)c1 ; OB(O)c1ccc2c(c1)c1ccccc1n2-c1cccc(-c2cc(-c3ccccn3)cc(-c3ccccn3)c2)c1]

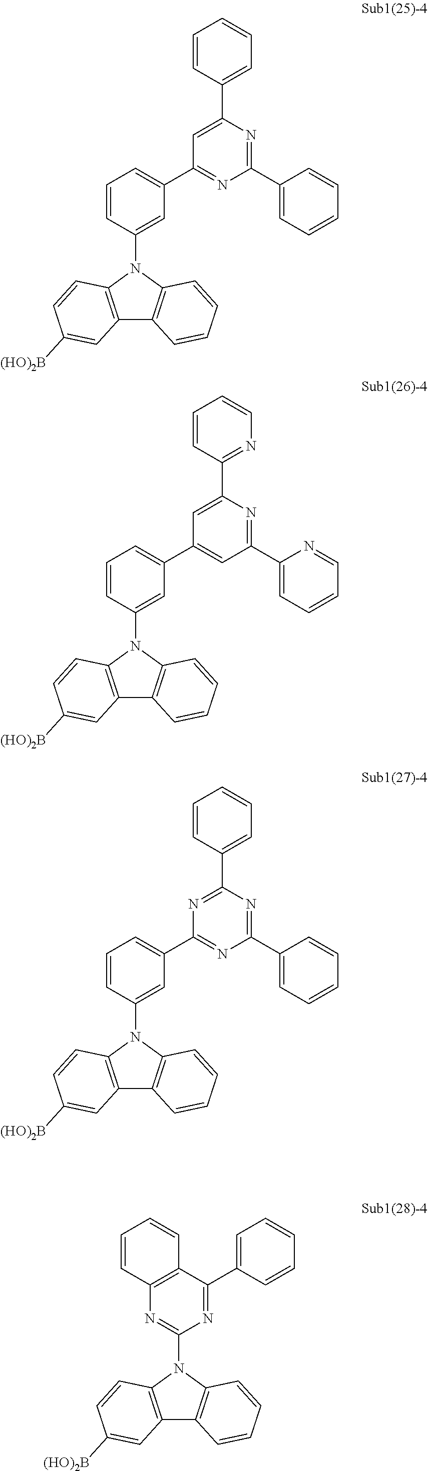 [SMILES: OB(O)c1ccc2c(c1)c1ccccc1n2-c1cccc(-c2cc(-c3ccccc3)nc(-c3ccccc3)n2)c1 ; OB(O)c1ccc2c(c1)c1ccccc1n2-c1cccc(-c2cc(-c3ccccn3)nc(-c3ccccn3)c2)c1 ; OB(O)c1ccc2c(c1)c1ccccc1n2-c1cccc(-c2nc(-c3ccccc3)nc(-c3ccccc3)n2)c1 ; OB(O)c1ccc2c(c1)c1ccccc1n2-c1nc(-c2ccccc2)c2ccccc2n1]

TABLE 4-2

| Compound | FD-MS | Compound | FD-MS |
|---|---|---|---|
| Sub 1(1)-4 | m/z = 287.11(C₁₈H₁₄BNO₂ = 287.12) | Sub 1(2)-4 | m/z = 337.13(C₂₂H₁₈BNO₂ = 337.18) |
| Sub 1(3)-4 | m/z = 363.14(C₂₄H₁₈BNO₂ = 363.22) | Sub 1(4)-4 | m/z = 363.14(C₂₄H₁₈BNO₂ = 363.22) |
| Sub 1(5)-4 | m/z = 442.16(C₂₇H₁₉BN₄O₂ = 442.28) | Sub 1(6)-4 | m/z = 441.16(C₂₅H₂₀BN₃O₂ = 441.29) |
| Sub 1(7)-4 | m/z = 441.16(C₂₃H₂₀BN₃O₂ = 441.29) | Sub 1(8)-4 | m/z = 441.16(C₂₈H₂₀BN₃O₂ = 441.29) |
| Sub 1(9)-4 | m/z = 440.17(C₃₉H₂₁BN₂O₂ = 440.30) | Sub 1(10)-4 | m/z = 440.17(C₂₉H₂₁BN₂O₂ = 440.30) |
| Sub 1(11)-4 | m/z = 441.16(C₂₈H₂₀BN₃O₂ = 441.29) | Sub 1(12)-4 | m/z = 442.16(C₂₇H₁₉BN₄O₂ = 442.28) |
| Sub 1(13)-4 | m/z = 517.20(C₃₄H₂₄BN₃O₂ = 517.38) | Sub 1(14)-4 | m/z = 516.20(C₃₅H₂₅BN₂O₂ = 516.40) |
| Sub 1(15)-4 | m/z = 516.20(C₃₅H₂₅BN₂O₂ = 516.40) | Sub 1(16)-4 | m/z = 517.20(C₃₄H₂₄BN₃O₂ = 517.38) |
| Sub 1(17)-4 | m/z = 518.19(C₃₅H₂₃BN₄O₂ = 518.37) | Sub 1(18)-4 | m/z = 517.20(C₃₄H₂₄BN₃O₂ = 517.38) |
| Sub 1(19)-4 | m/z = 517.20(C₃₄H₂₄BN₃O₂ = 517.38) | Sub 1(20)-4 | m/z = 518.19(C₃₃H₂₃BN₄O₂ = 518.37) |
| Sub 1(21)-4 | m/z = 517.20(C₃₄H₂₄BN₃O₂ = 517.38) | Sub 1(22)-4 | m/z = 516.20(C₃₅H₂₅BN₂O₂ = 516.40) |
| Sub 1(23)-4 | m/z = 516.20(C₃₅H₂₅BN₂O₂ = 516.40) | Sub 1(24)-4 | m/z = 517.20(C₃₄H₂₄BN₃O₂ = 517.38) |
| Sub 1(25)-4 | m/z = 517.20(C₃₄H₂₄BN₃O₂ = 517.38) | Sub 1(26)-4 | m/z = 518.19(C₃₃H₂₃BN₄O₂ = 518.37) |
| Sub 1(27)-4 | m/z = 518.19(C₃₃H₂₃BN₄O₂ = 518.37) | Sub 1(28)-4 | m/z = 415.15(C₂₆H₁₈BN₃O₂ = 415.25) |

### II. Synthesis Example of Sub 2-4

Sub 2-4 in Reaction Scheme 4-1 may be synthesized via the reaction pathway of Reaction Scheme 4-5 below, but is not limited thereto.

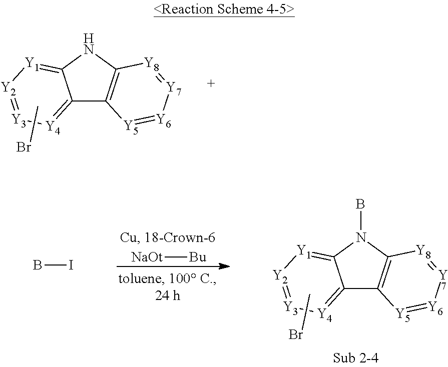

### Synthesis of Sub 2-1(1)-4

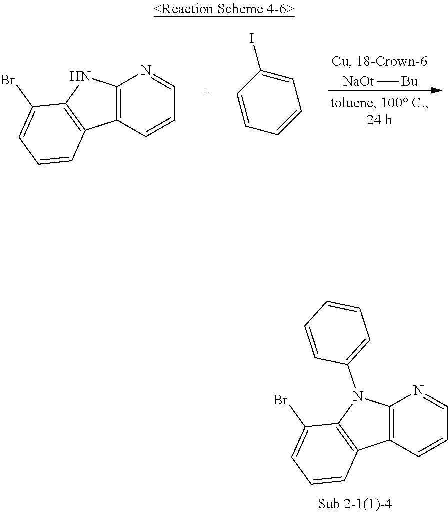

After 8-bromo-9H-pyrido[2,3-b]indole (50.2 g, 203 mmol) and iodobenzene (49.0 g, 240 mmol) were mixed with 800 mL of toluene, Cu (764 mg, 12 mmol), 18-Crown-6 (6.3 g, 24 mmol), and NaOt-Bu (57.6 g, 600 mmol) were added thereto, and the mixture was stirred under reflux at 1000 for 24 h. After extraction with ether and water, the organic layer was dried over MgSO₄ and concentrated, and then the generated organic material was subjected to silica gel column chromatography and recrystallization to give 28.2 g of 8-bromo-9-phenyl-9H-pyrido[2,3-b]indole (yield: 43%).

Examples of Sub 2-4 are as follows, but are limited thereto, and FD-MS values thereof are shown in table 4-3 below.

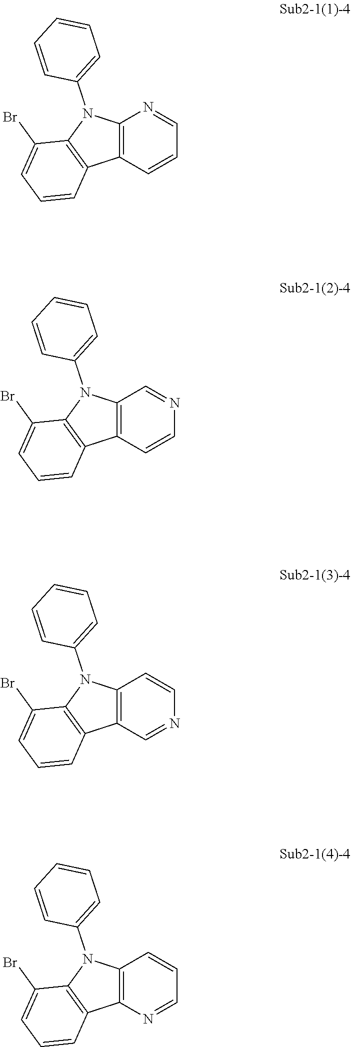 [SMILES: Brc1cccc2c3cccnc3n(-c3ccccc3)c12 ; Brc1cccc2c3ccncc3n(-c3ccccc3)c12 ; Brc1cccc2c3cnccc3n(-c3ccccc3)c12 ; Brc1cccc2c3ncccc3n(-c3ccccc3)c12]

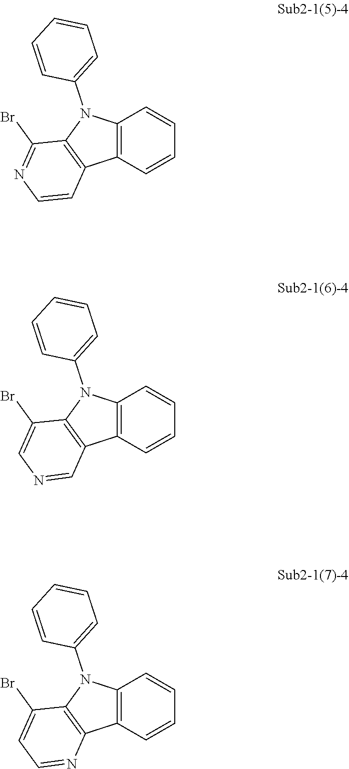 [SMILES: Brc1nccc2c3ccccc3n(-c3ccccc3)c12 ; Brc1cncc2c3ccccc3n(-c3ccccc3)c12 ; Brc1ccnc2c3ccccc3n(-c3ccccc3)c12]

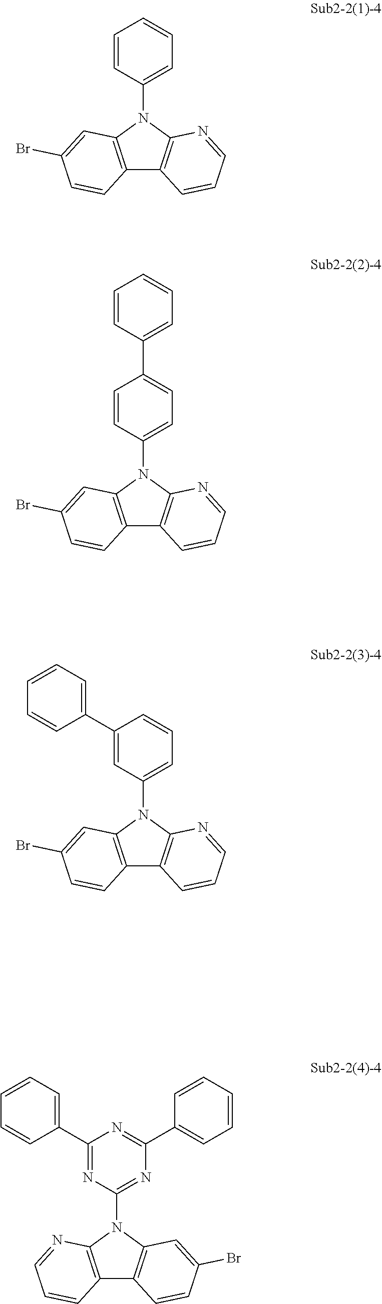 [SMILES: Brc1ccc2c3cccnc3n(-c3ccccc3)c2c1 ; Brc1ccc2c3cccnc3n(-c3ccc(-c4ccccc4)cc3)c2c1 ; Brc1ccc2c3cccnc3n(-c3cccc(-c4ccccc4)c3)c2c1 ; Brc1ccc2c3cccnc3n(-c3nc(-c4ccccc4)nc(-c4ccccc4)n3)c2c1]

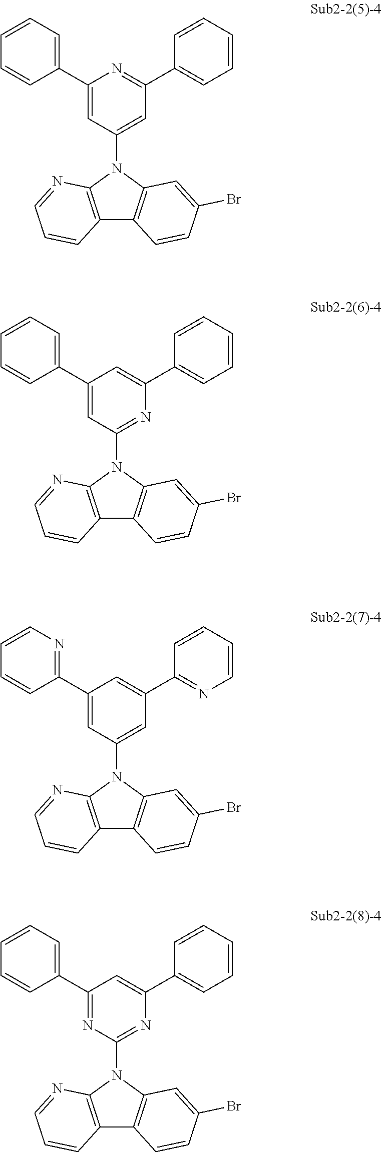 [SMILES: Brc1ccc2c3cccnc3n(-c3cc(-c4ccccc4)nc(-c4ccccc4)c3)c2c1 ; Brc1ccc2c3cccnc3n(-c3cc(-c4ccccc4)cc(-c4ccccc4)n3)c2c1 ; Brc1ccc2c3cccnc3n(-c3cc(-c4ccccn4)cc(-c4ccccn4)c3)c2c1 ; Brc1ccc2c3cccnc3n(-c3nc(-c4ccccc4)cc(-c4ccccc4)n3)c2c1]

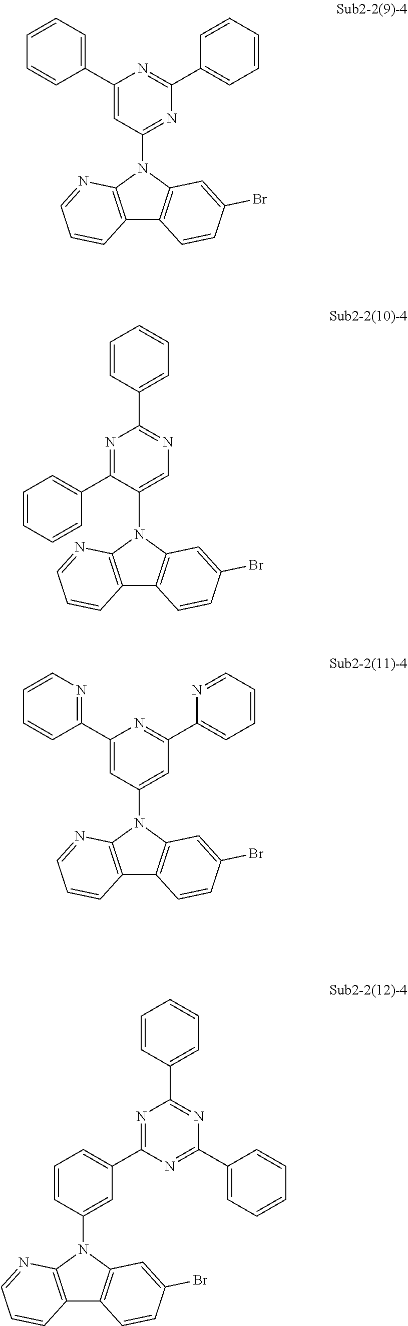 [SMILES: Brc1ccc2c3cccnc3n(-c3cc(-c4ccccc4)nc(-c4ccccc4)n3)c2c1 ; Brc1ccc2c3cccnc3n(-c3cnc(-c4ccccc4)nc3-c3ccccc3)c2c1 ; Brc1ccc2c3cccnc3n(-c3cc(-c4ccccn4)nc(-c4ccccn4)c3)c2c1 ; Brc1ccc2c3cccnc3n(-c3cccc(-c4nc(-c5ccccc5)nc(-c5ccccc5)n4)c3)c2c1]

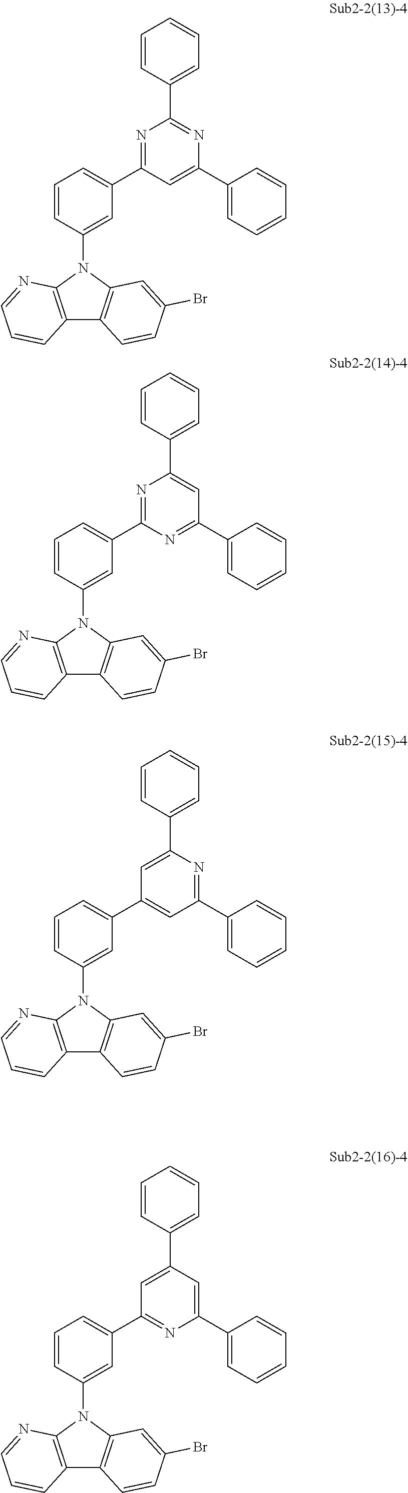

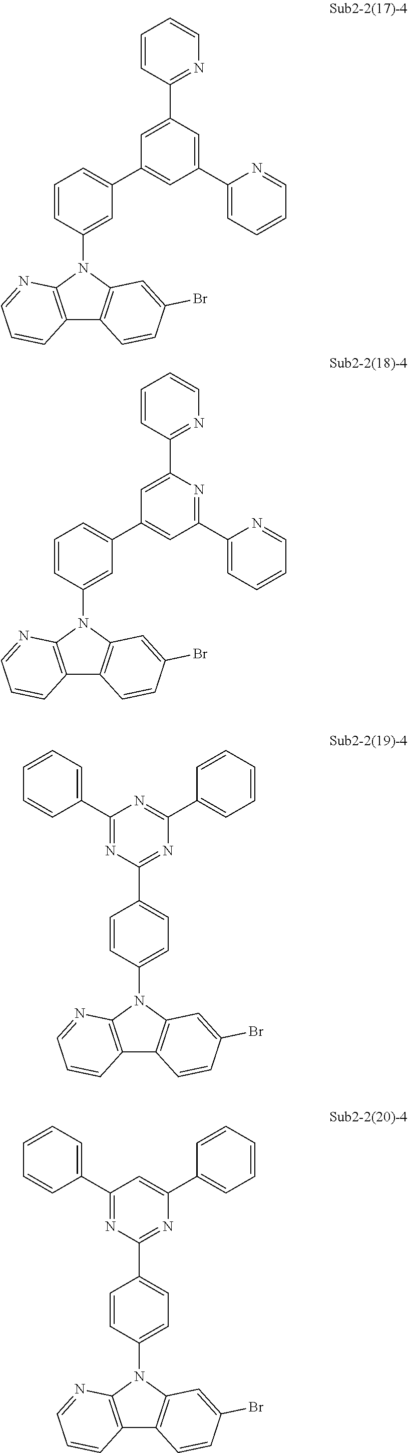

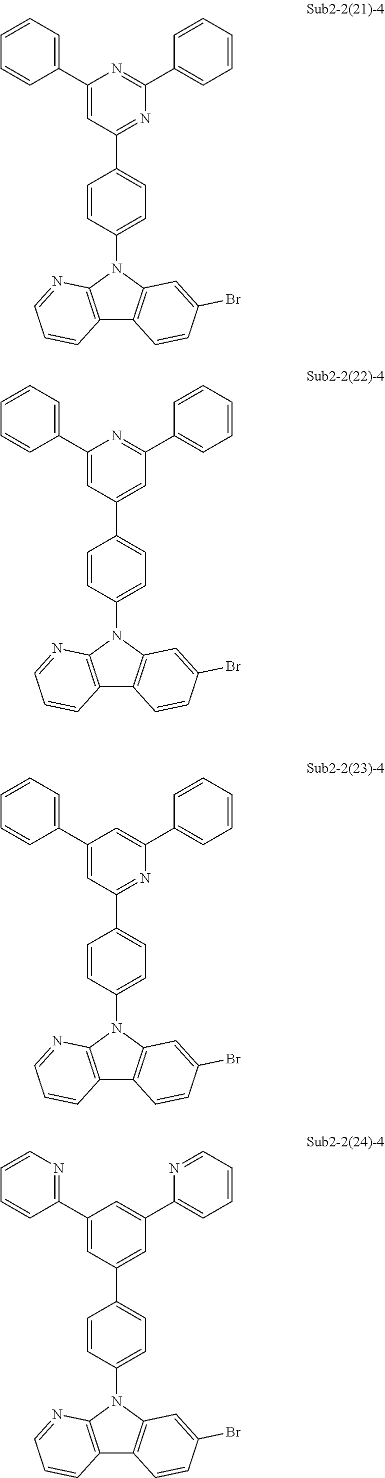 [SMILES: Brc1ccc2c3cccnc3n(-c3ccc(-c4cc(-c5ccccc5)nc(-c5ccccc5)n4)cc3)c2c1 ; Brc1ccc2c3cccnc3n(-c3ccc(-c4cc(-c5ccccc5)nc(-c5ccccc5)c4)cc3)c2c1 ; Brc1ccc2c3cccnc3n(-c3ccc(-c4cc(-c5ccccc5)cc(-c5ccccc5)n4)cc3)c2c1 ; Brc1ccc2c3cccnc3n(-c3ccc(-c4cc(-c5ccccn5)cc(-c5ccccn5)c4)cc3)c2c1]

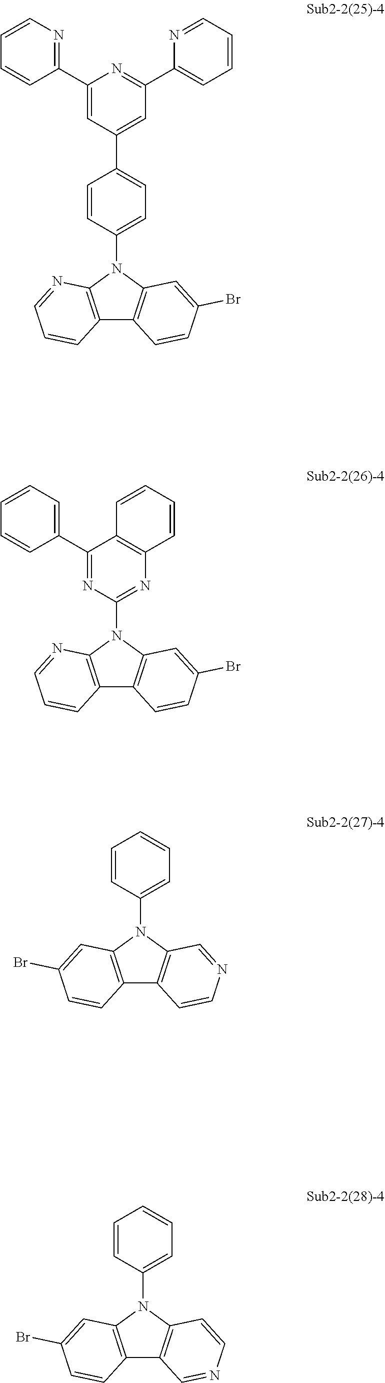 [SMILES: Brc1ccc2c3cccnc3n(-c3ccc(-c4cc(-c5ccccn5)nc(-c5ccccn5)c4)cc3)c2c1 ; Brc1ccc2c3cccnc3n(-c3nc(-c4ccccc4)c4ccccc4n3)c2c1 ; Brc1ccc2c3ccncc3n(-c3ccccc3)c2c1 ; Brc1ccc2c3cnccc3n(-c3ccccc3)c2c1]

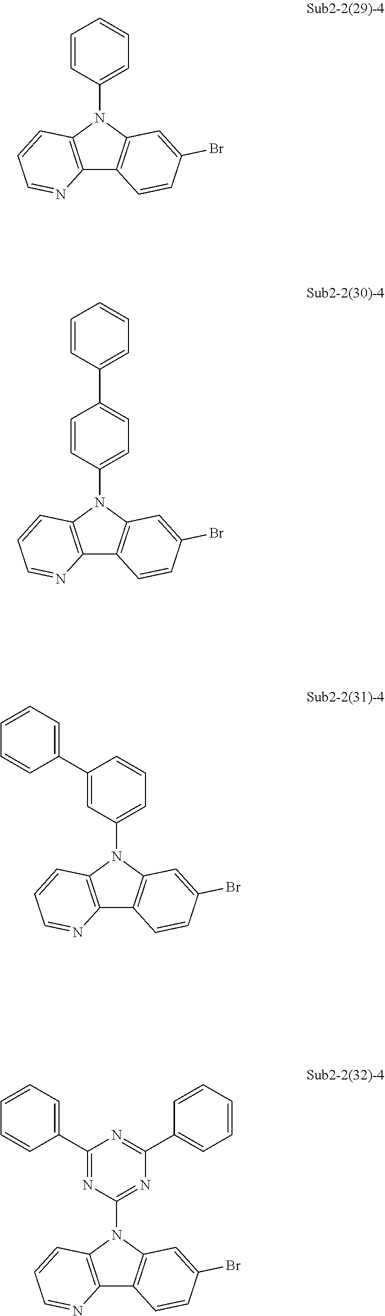 [SMILES: Brc1ccc2c3ncccc3n(-c3ccccc3)c2c1 ; Brc1ccc2c3ncccc3n(-c3ccc(-c4ccccc4)cc3)c2c1 ; Brc1ccc2c3ncccc3n(-c3cccc(-c4ccccc4)c3)c2c1 ; Brc1ccc2c3ncccc3n(-c3nc(-c4ccccc4)nc(-c4ccccc4)n3)c2c1]

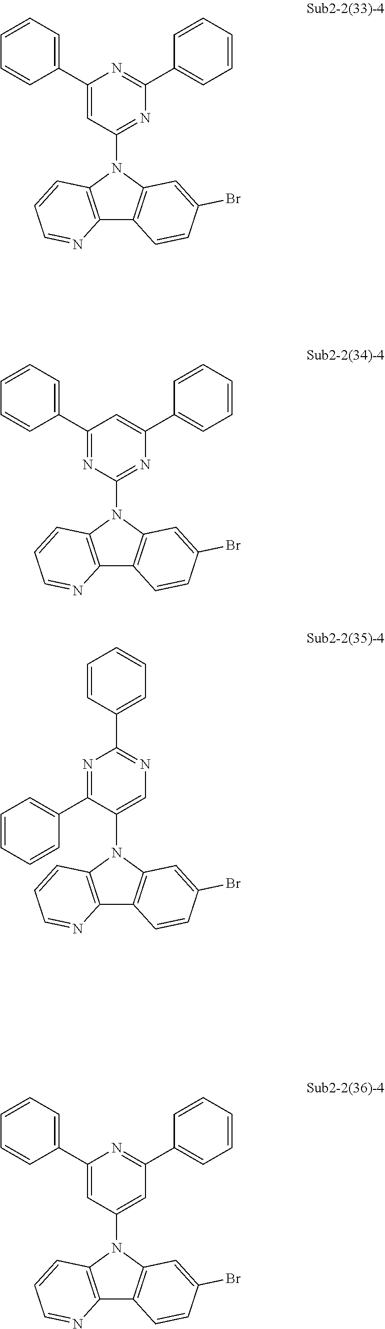 [SMILES: Brc1ccc2c3ncccc3n(-c3cc(-c4ccccc4)nc(-c4ccccc4)n3)c2c1 ; Brc1ccc2c3ncccc3n(-c3nc(-c4ccccc4)cc(-c4ccccc4)n3)c2c1 ; Brc1ccc2c3ncccc3n(-c3cnc(-c4ccccc4)nc3-c3ccccc3)c2c1 ; Brc1ccc2c3ncccc3n(-c3cc(-c4ccccc4)nc(-c4ccccc4)c3)c2c1]

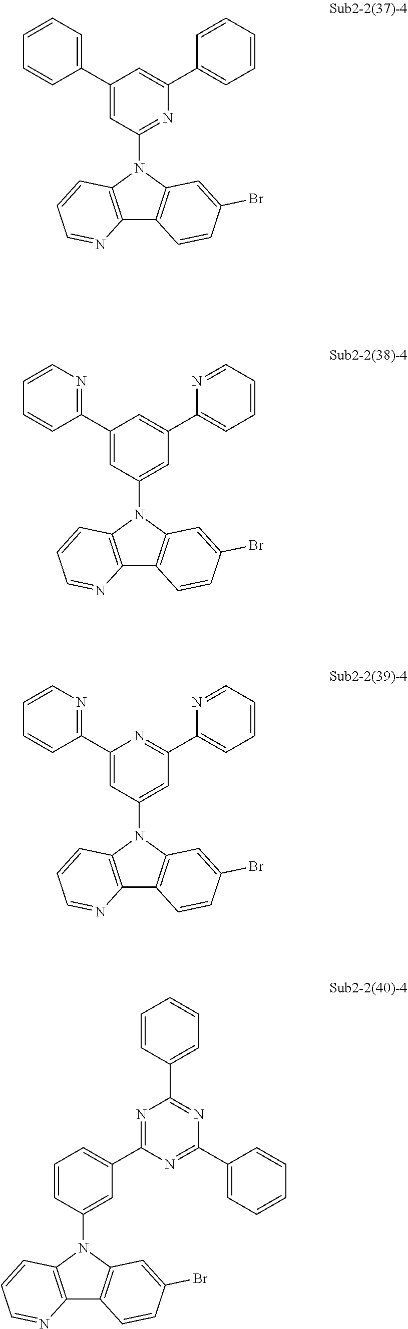 [SMILES: Brc1ccc2c3ncccc3n(-c3cc(-c4ccccc4)cc(-c4ccccc4)n3)c2c1 ; Brc1ccc2c3ncccc3n(-c3cc(-c4ccccn4)cc(-c4ccccn4)c3)c2c1 ; Brc1ccc2c3ncccc3n(-c3cc(-c4ccccn4)nc(-c4ccccn4)c3)c2c1 ; Brc1ccc2c3ncccc3n(-c3cccc(-c4nc(-c5ccccc5)nc(-c5ccccc5)n4)c3)c2c1]

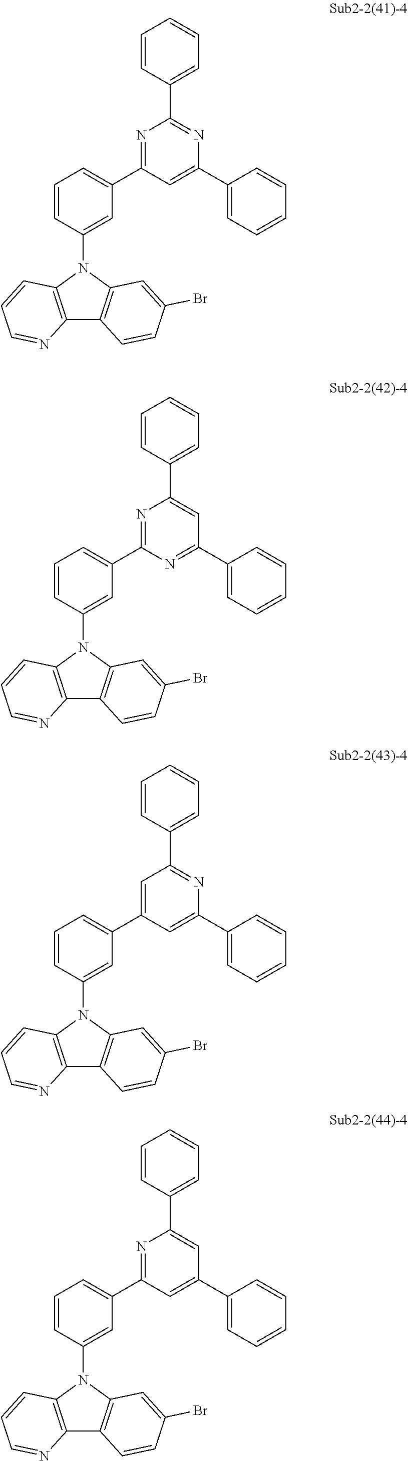 [SMILES: Brc1ccc2c3ncccc3n(-c3cccc(-c4cc(-c5ccccc5)nc(-c5ccccc5)n4)c3)c2c1 ; Brc1ccc2c3ncccc3n(-c3cccc(-c4nc(-c5ccccc5)cc(-c5ccccc5)n4)c3)c2c1 ; Brc1ccc2c3ncccc3n(-c3cccc(-c4cc(-c5ccccc5)nc(-c5ccccc5)c4)c3)c2c1 ; Brc1ccc2c3ncccc3n(-c3cccc(-c4cc(-c5ccccc5)cc(-c5ccccc5)n4)c3)c2c1]

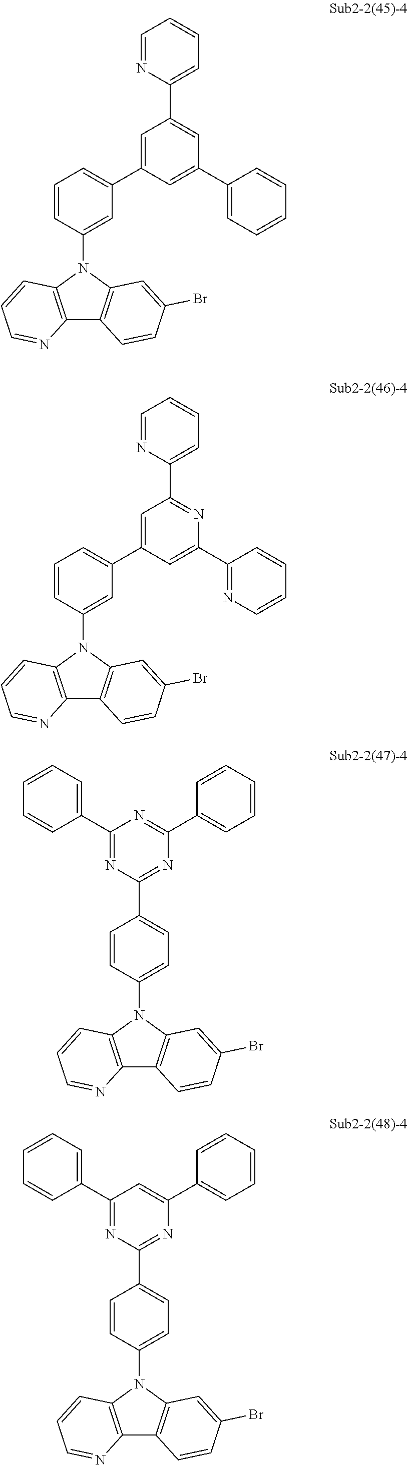

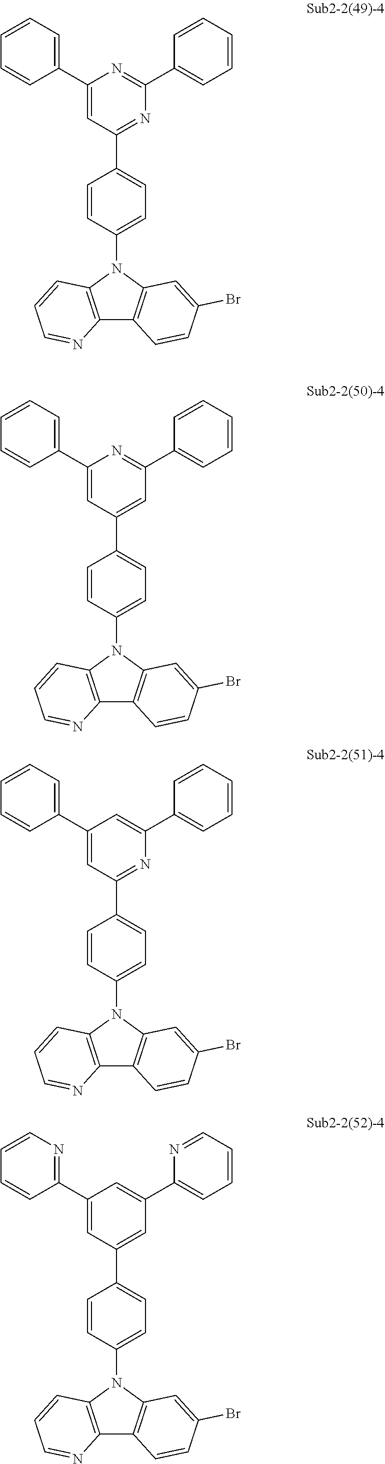 [SMILES: Brc1ccc2c3ncccc3n(-c3ccc(-c4cc(-c5ccccc5)nc(-c5ccccc5)n4)cc3)c2c1 ; Brc1ccc2c3ncccc3n(-c3ccc(-c4cc(-c5ccccc5)nc(-c5ccccc5)c4)cc3)c2c1 ; Brc1ccc2c3ncccc3n(-c3ccc(-c4cc(-c5ccccc5)cc(-c5ccccc5)n4)cc3)c2c1 ; Brc1ccc2c3ncccc3n(-c3ccc(-c4cc(-c5ccccn5)cc(-c5ccccn5)c4)cc3)c2c1]

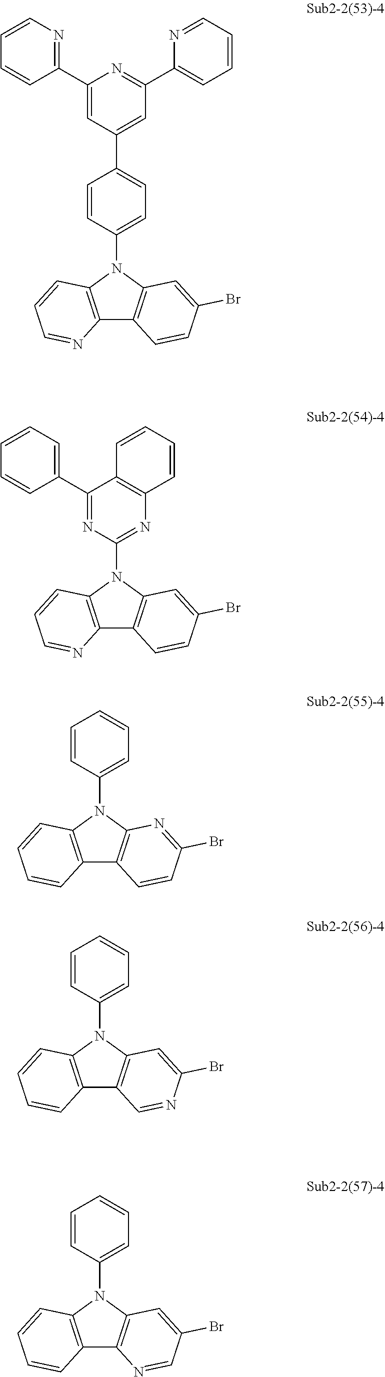 [SMILES: Brc1ccc2c3ncccc3n(-c3ccc(-c4cc(-c5ccccn5)nc(-c5ccccn5)c4)cc3)c2c1 ; Brc1ccc2c3ncccc3n(-c3nc(-c4ccccc4)c4ccccc4n3)c2c1 ; Brc1ccc2c3ccccc3n(-c3ccccc3)c2n1 ; Brc1cc2c(cn1)c1ccccc1n2-c1ccccc1 ; Brc1cnc2c3ccccc3n(-c3ccccc3)c2c1]

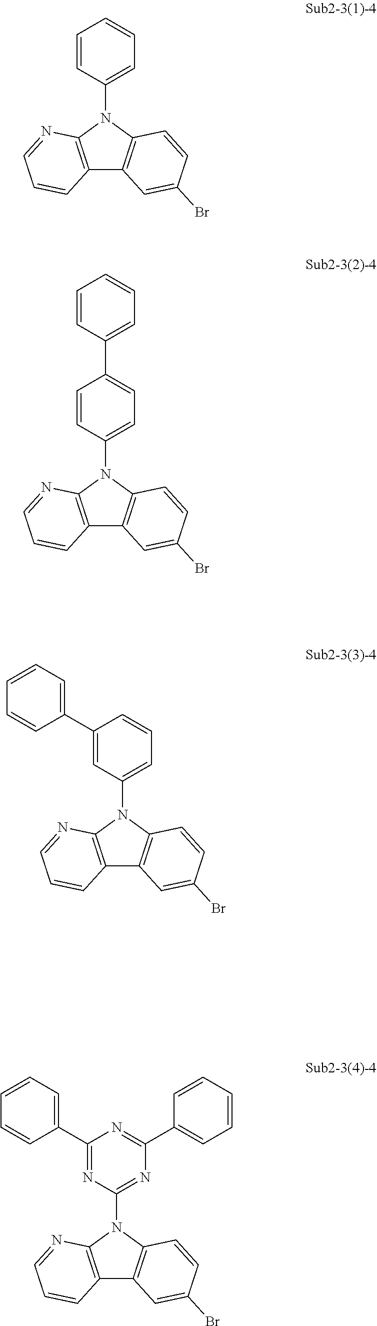 [SMILES: Brc1ccc2c(c1)c1cccnc1n2-c1ccccc1 ; Brc1ccc2c(c1)c1cccnc1n2-c1ccc(-c2ccccc2)cc1 ; Brc1ccc2c(c1)c1cccnc1n2-c1cccc(-c2ccccc2)c1 ; Brc1ccc2c(c1)c1cccnc1n2-c1nc(-c2ccccc2)nc(-c2ccccc2)n1]

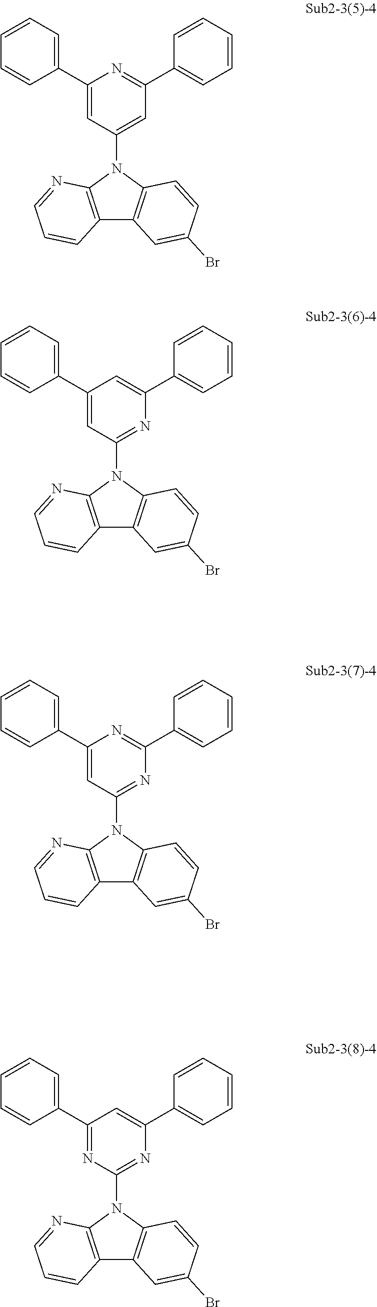 [SMILES: Brc1ccc2c(c1)c1cccnc1n2-c1cc(-c2ccccc2)nc(-c2ccccc2)c1 ; Brc1ccc2c(c1)c1cccnc1n2-c1cc(-c2ccccc2)cc(-c2ccccc2)n1 ; Brc1ccc2c(c1)c1cccnc1n2-c1cc(-c2ccccc2)nc(-c2ccccc2)n1 ; Brc1ccc2c(c1)c1cccnc1n2-c1nc(-c2ccccc2)cc(-c2ccccc2)n1]

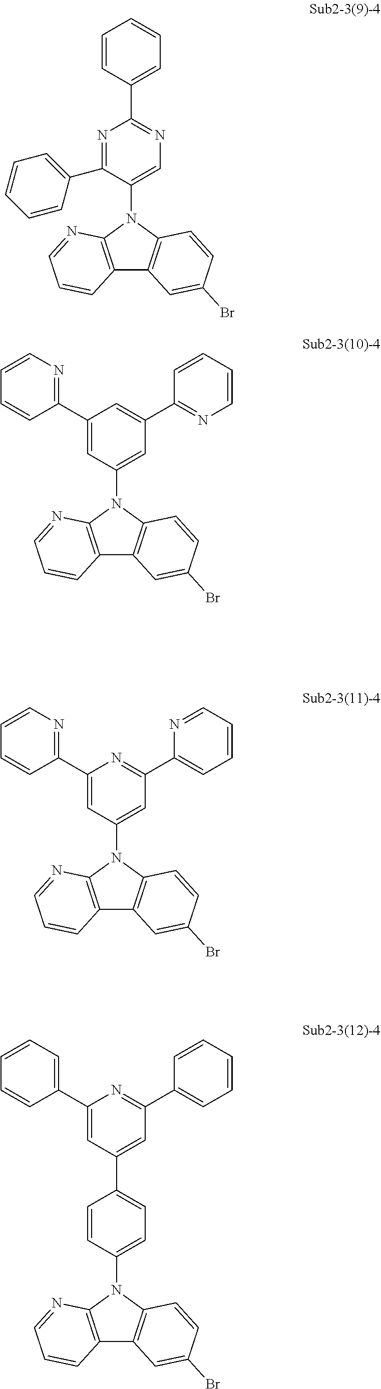

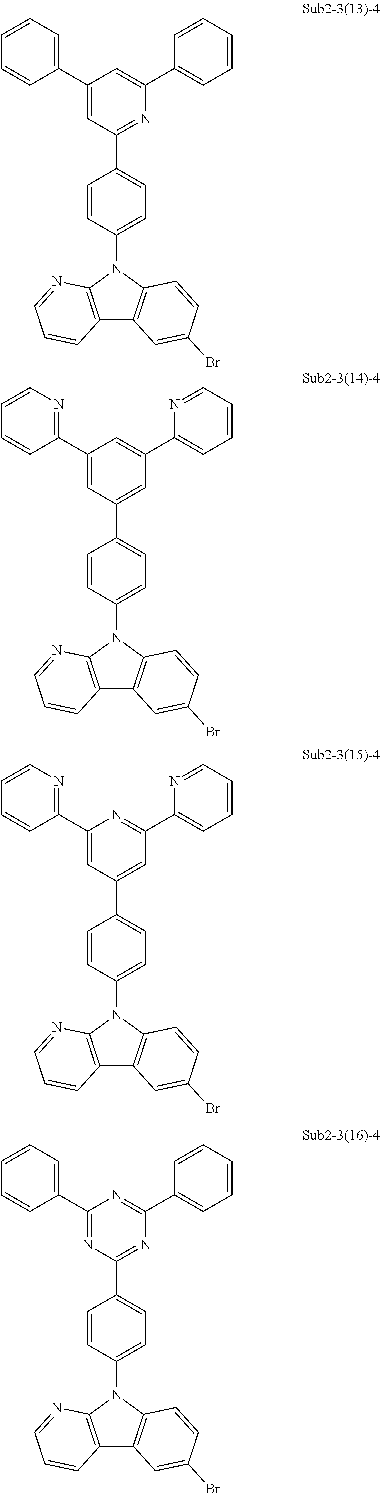

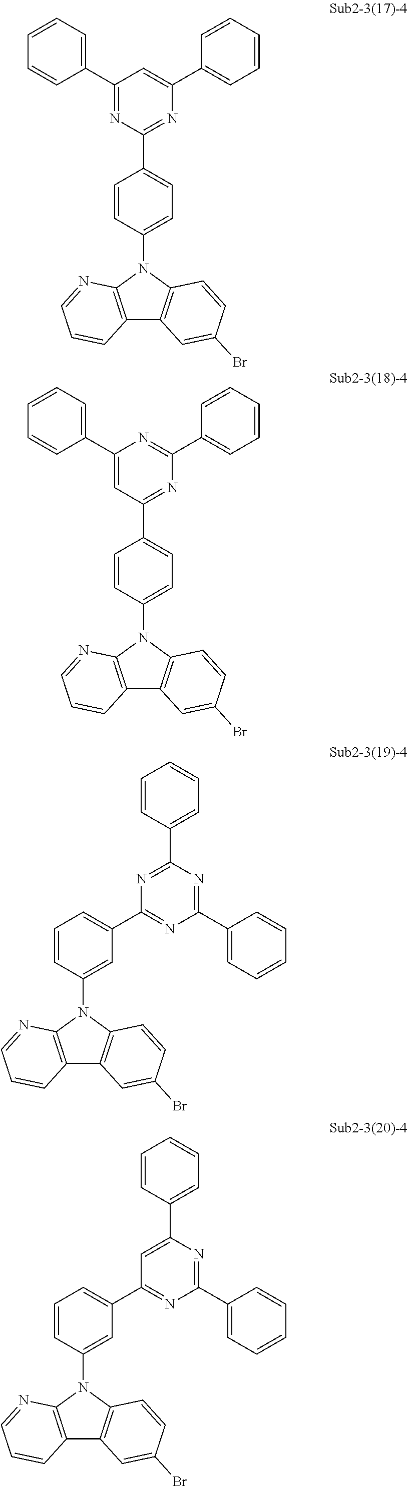

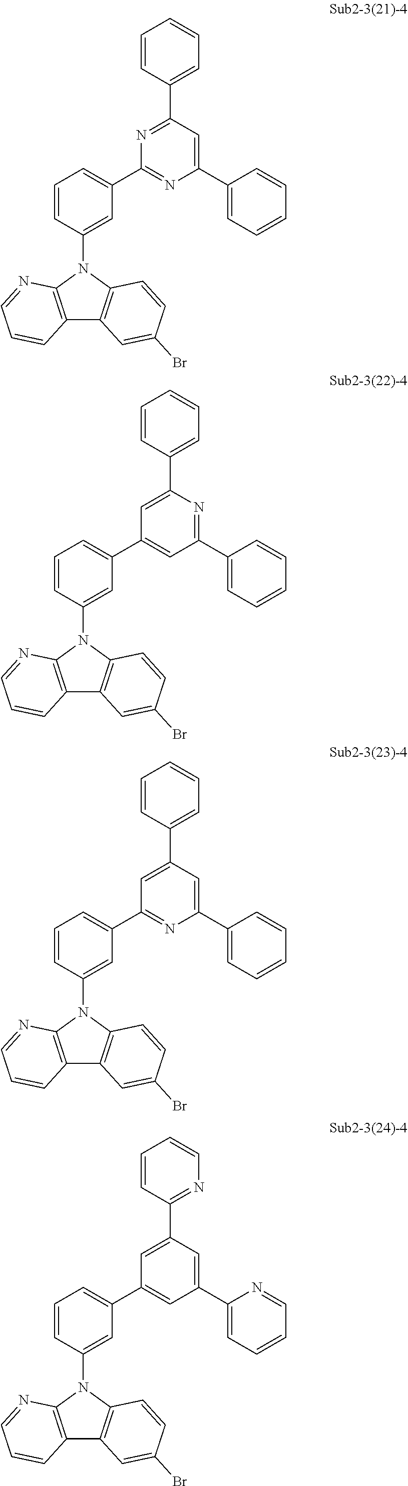

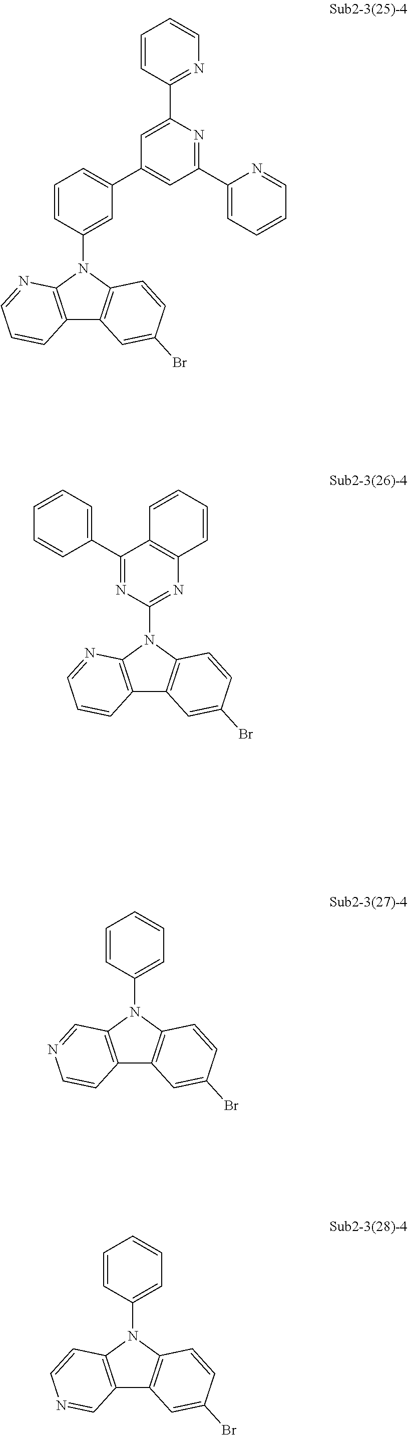 [SMILES: Brc1ccc2c(c1)c1cccnc1n2-c1cccc(-c2cc(-c3ccccn3)nc(-c3ccccn3)c2)c1 ; Brc1ccc2c(c1)c1cccnc1n2-c1nc(-c2ccccc2)c2ccccc2n1 ; Brc1ccc2c(c1)c1ccncc1n2-c1ccccc1 ; Brc1ccc2c(c1)c1cnccc1n2-c1ccccc1]

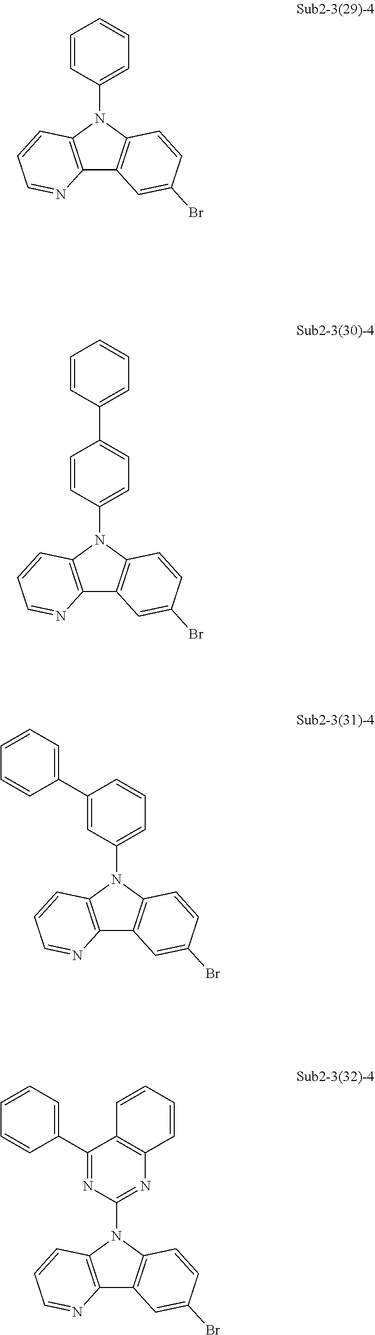 [SMILES: Brc1ccc2c(c1)c1ncccc1n2-c1ccccc1 ; Brc1ccc2c(c1)c1ncccc1n2-c1ccc(-c2ccccc2)cc1 ; Brc1ccc2c(c1)c1ncccc1n2-c1cccc(-c2ccccc2)c1 ; Brc1ccc2c(c1)c1ncccc1n2-c1nc(-c2ccccc2)c2ccccc2n1]

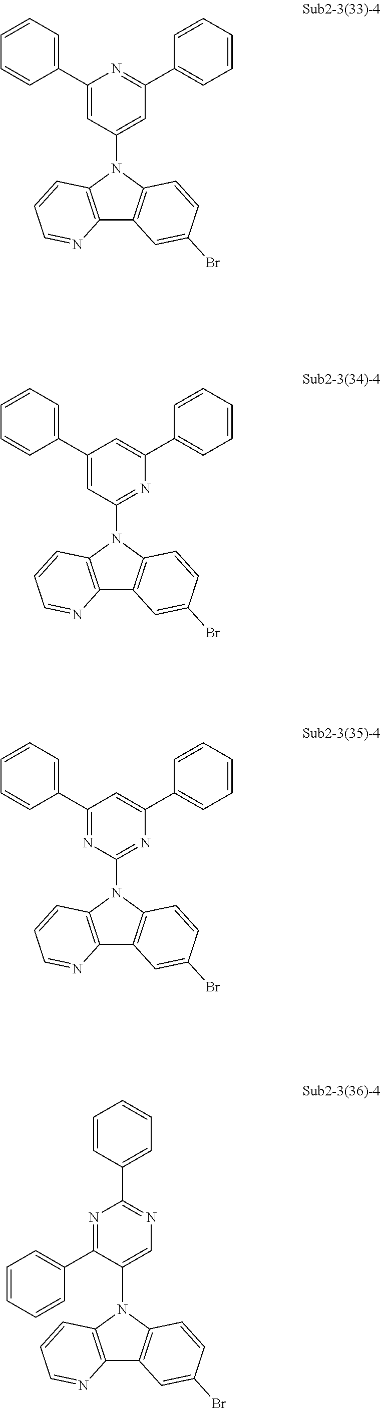 [SMILES: Brc1ccc2c(c1)c1ncccc1n2-c1cc(-c2ccccc2)nc(-c2ccccc2)c1 ; Brc1ccc2c(c1)c1ncccc1n2-c1cc(-c2ccccc2)cc(-c2ccccc2)n1 ; Brc1ccc2c(c1)c1ncccc1n2-c1nc(-c2ccccc2)cc(-c2ccccc2)n1 ; Brc1ccc2c(c1)c1ncccc1n2-c1cnc(-c2ccccc2)nc1-c1ccccc1]

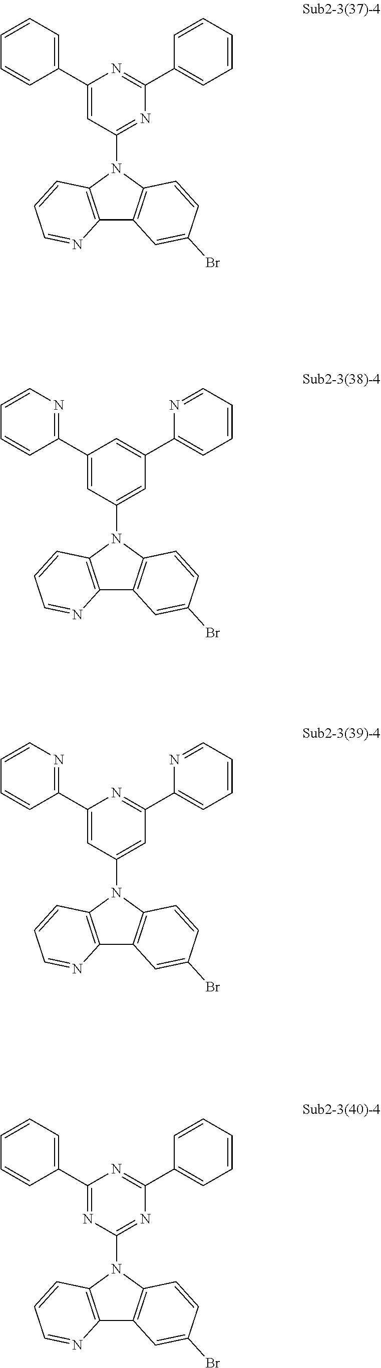 [SMILES: Brc1ccc2c(c1)c1ncccc1n2-c1cc(-c2ccccc2)nc(-c2ccccc2)n1 ; Brc1ccc2c(c1)c1ncccc1n2-c1cc(-c2ccccn2)cc(-c2ccccn2)c1 ; Brc1ccc2c(c1)c1ncccc1n2-c1cc(-c2ccccn2)nc(-c2ccccn2)c1 ; Brc1ccc2c(c1)c1ncccc1n2-c1nc(-c2ccccc2)nc(-c2ccccc2)n1]

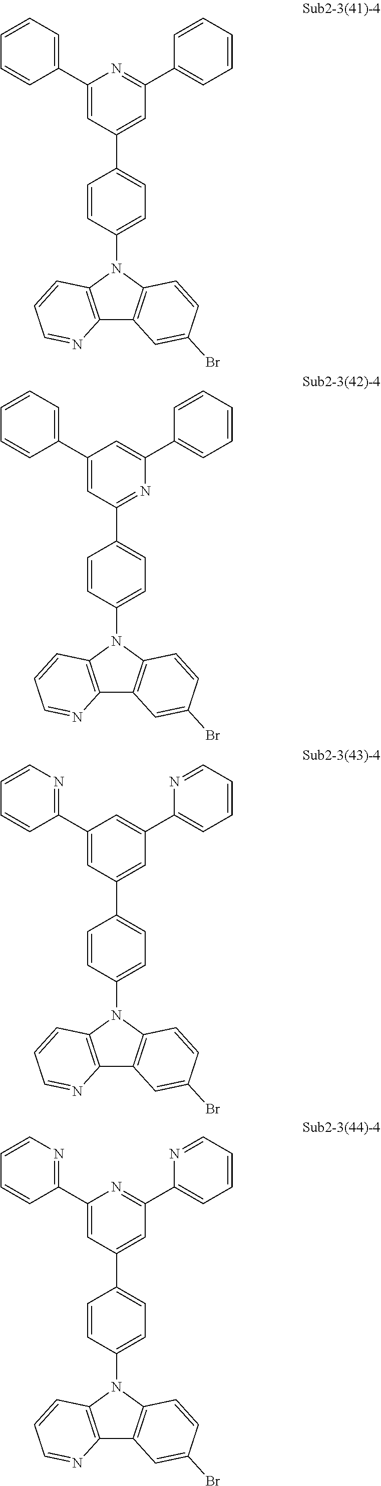

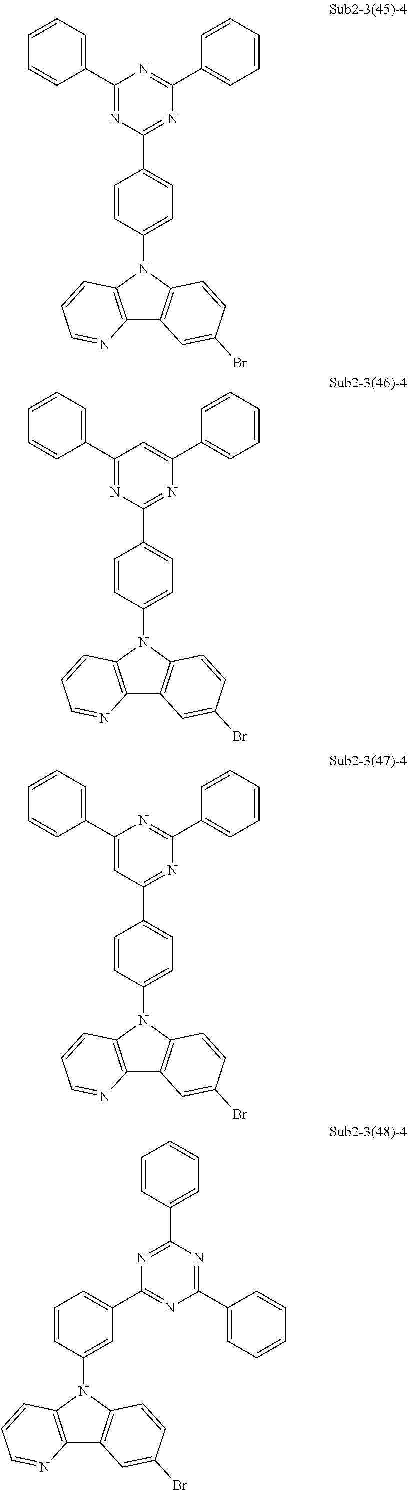

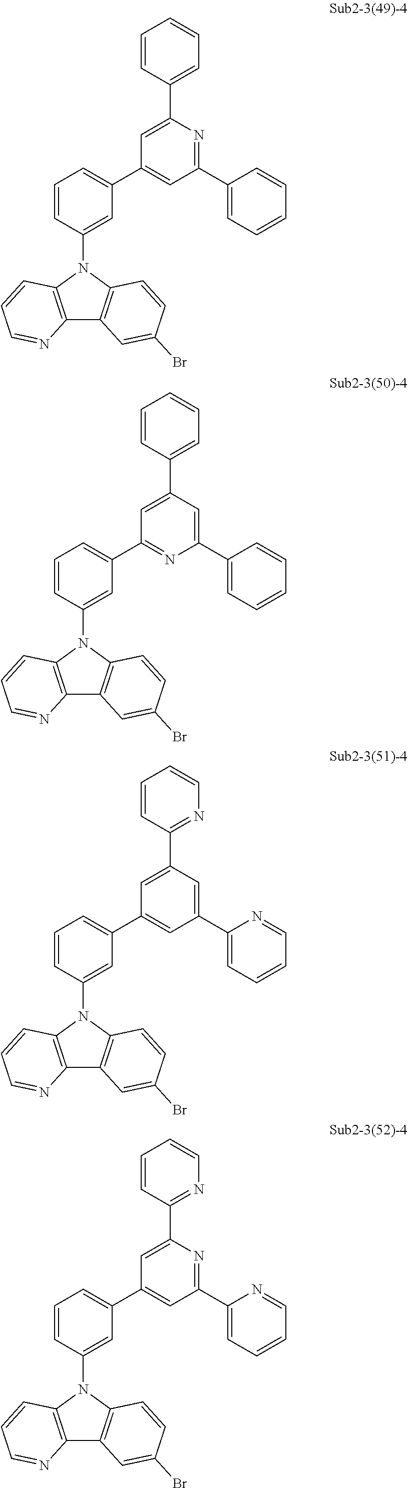

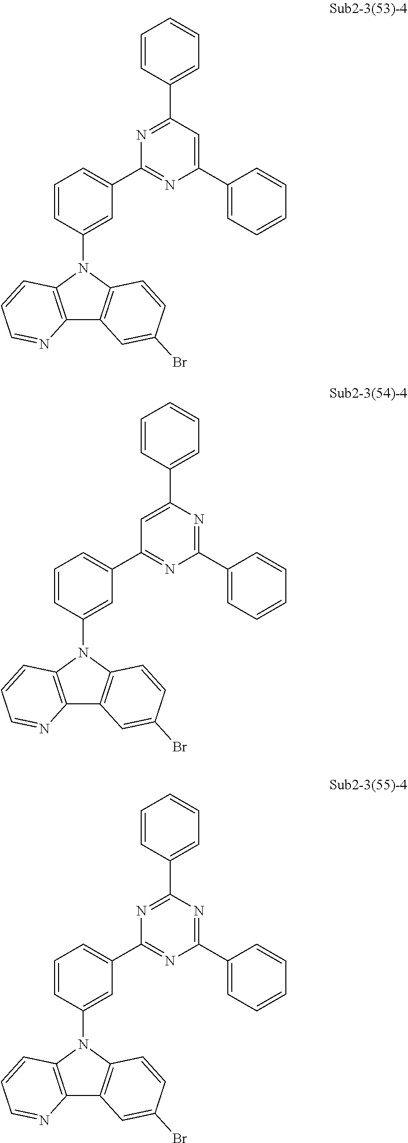

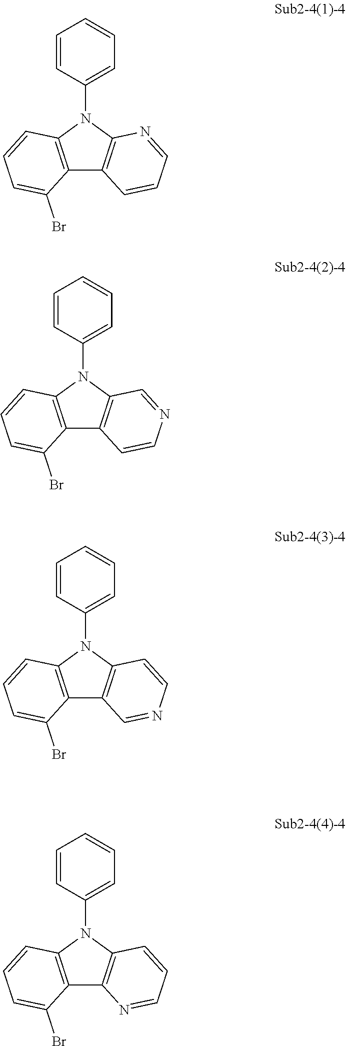 [SMILES: Brc1cccc2c1c1cccnc1n2-c1ccccc1 ; Brc1cccc2c1c1ccncc1n2-c1ccccc1 ; Brc1cccc2c1c1cnccc1n2-c1ccccc1 ; Brc1cccc2c1c1ncccc1n2-c1ccccc1]

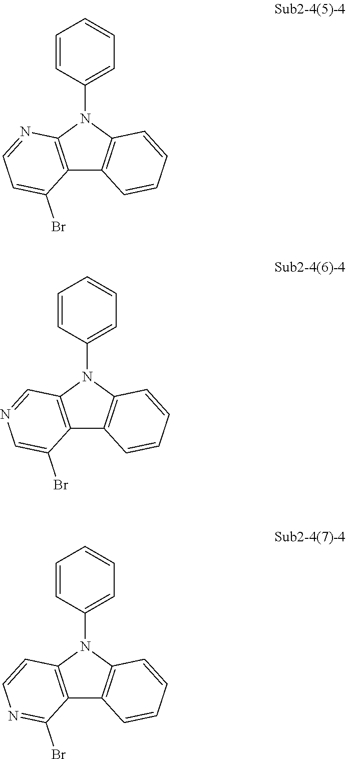 [SMILES: Brc1ccnc2c1c1ccccc1n2-c1ccccc1 ; Brc1cncc2c1c1ccccc1n2-c1ccccc1 ; Brc1nccc2c1c1ccccc1n2-c1ccccc1]

TABLE 4-3

| Compound | FD-MS | Compound | FD-MS |
|---|---|---|---|
| Sub2-1(1)-4 | m/z = 322.01(C₁₇H₁₁BrN₂ = 323.19) | Sub2-1(2)-4 | m/z = 322.01(C₁₇H₁₁BrN₂ = 323.19) |
| Sub2-1(3)-4 | m/z = 322.01(C₁₇H₁₁BrN₂ = 323.19) | Sub2-1(4)-4 | m/z = 322.01(C₁₇H₁₁BrN₂ = 323.19) |
| Sub2-1(5)-4 | m/z = 322.01(C₁₇H₁₁BrN₂ = 323.19) | Sub2-1(6)-4 | m/z = 322.01(C₁₇H₁₁BrN₂ = 323.19) |
| Sub2-1(7)-4 | m/z = 322.01(C₁₇H₁₁BrN₂ = 323.19) | Sub2-2(1)-4 | m/z = 322.01(C₁₇H₁₁BrN₂ = 323.19) |
| Sub2-2(2)-4 | m/z = 398.04(C₂₃H₁₅BrN₂ = 399.28) | Sub2-2(3)-4 | m/z = 398.04(C₂₃H₁₅BrN₂ = 399.28) |
| Sub2-2(4)-4 | m/z = 477.06(C₂₆H₁₆BrN₅ = 478.34) | Sub2-2(5)-4 | m/z = 475.07(C₂₈H₁₈BrN₃ = 476.37) |
| Sub2-2(6)-4 | m/z = 475.07(C₂₈H₁₈BrN₃ = 476.37) | Sub2-2(7)-4 | m/z = 476.06(C₂₈H₁₈BrN₃ = 477.35) |
| Sub2-2(8)-4 | m/z = 476.06(C₂₈H₁₈BrN₃ = 477.35) | Sub2-2(9)-4 | m/z = 476.06(C₂₈H₁₈BrN₃ = 477.35) |
| Sub2-2(10)-4 | m/z = 476.06(C₂₈H₁₈BrN₅ = 477.35) | Sub2-2(11)-4 | m/z = 477.06(C₂₆H₁₆BrN₅ = 478.34) |
| Sub2-2(12)-4 | m/z = 553.09(C₃₂H₂₀BrN₅ = 554.44) | Sub2-2(13)-4 | m/z = 552.09(C₃₃H₂₁BrN₄ = 553.45) |
| Sub2-2(14)-4 | m/z = 552.09(C₃₃H₂₁BrN₄ = 553.45) | Sub2-2(15)-4 | m/z = 551.10(C₃₄H₂₂BrN₃ = 552.46) |
| Sub2-2(16)-4 | m/z = 551.10(C₃₄H₂2BrN₃ = 552.46) | Sub2-2(17)-4 | m/z = 552.09(C₃₃H₂₁BrN₄ = 553.45) |
| Sub2-2(18)-4 | m/z = 553.09(C₃₂H₂₀BrN₅ = 554.44) | Sub2-2(19)-4 | m/z = 553.09(C₃₂H₂₀BrN₅ = 554.44) |
| Sub2-2(20)-4 | m/z = 552.09(C₃₃H₂₂BrN₄ = 553.45) | Sub2-2(21)-4 | m/z = 552.09(C₃₃H₂₁BrN₄ = 553.45) |
| Sub2-2(22)-4 | m/z = 551.10(C₃₄H₂₂BrN₃ = 552.46) | Sub2-2(23)-4 | m/z = 551.10(C₃₄H₂₂BrN₃ = 552.46) |
| Sub2-2(24)-4 | m/z = 552.09(C₃₃H₂₁BrN₄ = 553.45) | Sub2-2(25)-4 | m/z = 552.09(C₃₃H₂₁BrN₄ = 553.45) |
| Sub2-2(26)-4 | m/z = 450.05(C₂₅H₁₅BrN₄ = 451.32) | Sub2-2(27)-4 | m/z = 322.01(C₁₇H₁₁BrN₂ = 323.19) |
| Sub2-2(28)-4 | m/z = 322.01(C₁₇H₁₇BrN₂ = 323.19) | Sub2-2(29)-4 | m/z = 322.01(C₁₇H₁₁BrN₂ = 323.19) |
| Sub2-2(30)-4 | m/z = 398.04(C₃₃H₁₅BrN₂ = 399.28) | Sub2-2(31)-4 | m/z = 398.04(C₂₃H₁₅BrN₂ = 399.28) |
| Sub2-2(32)-4 | m/z = 477.06(C₂₆H₁₆BrN₅ = 478.34) | Sub2-2(33)-4 | m/z = 476.06(C₂₈H₁₈BrN₃ = 477.35) |
| Sub2-2(33)-4 | m/z = 476.06(C₂₈H₁₈BrN₃ = 477.35) | Sub2-2(35)-4 | m/z = 476.06(C₂₈H₁₈BrN₃ = 477.35) |
| Sub2-2(36)-4 | m/z = 475.07(C₂₈H₁₈BrN₃ = 476.37) | Sub2-2(37)-4 | m/z = 475.07(C₂₈H₁₈BrN₃ = 476.37) |
| Sub2-2(38)-4 | m/z = 476.06(C₂₈H₁₈BrN₃ = 477.35) | Sub2-2(39)-4 | m/z = 476.06(C₂₈H₁₈BrN₃ = 477.35) |
| Sub2-2(40)-4 | m/z = 553.09(C₃₂H₂₀BrN₅ = 554.44) | Sub2-2(41)-4 | m/z = 552.09(C₃₃H₂₁BrN₄ = 553.45) |
| Sub2-2(42)-4 | m/z = 552.09(C₃₃H₂₁BrN₄ = 553.45) | Sub2-2(43)-4 | m/z = 551.10(C₃₄H₂₂BrN₃ = 552.46) |
| Sub2-2(44)-4 | m/z = 551.10(C₃₄H₂₂BrN₃ = 552.46) | Sub2-2(45)-4 | m/z = 552.09(C₃₃H₂₁BrN₄ = 553.45) |
| Sub2-2(46)-4 | m/z = 553.09(C₃₂H₃₀BrN₅ = 554.44) | Sub2-2(47)-4 | m/z = 553.09(C₃₂H₂₀BrN₅ = 554.44) |
| Sub2-2(48)-4 | m/z = 552.09(C₃₃H₂₁BrN₄ = 553.45) | Sub2-2(49)-4 | m/z = 552.09(C₃₃H₂₁BrN₄ = 553.45) |
| Sub2-2(50)-4 | m/z = 551.10(C₃₄H₂₂BrN₃ = 552.46) | Sub2-2(51)-4 | m/z = 551.10(C₃₄H₂₂BrN₃ = 552.46) |
| Sub2-2(52)-4 | m/z = 552.09(C₃₃H₂₂BrN₄ = 553.45) | Sub2-2(53)-4 | m/z = 553.09(C₃₂H₂₀BrN₅ = 554.44) |
| Sub2-2(54)-4 | m/z = 450.05(C₂₅H₁₅BrN₄ = 451.32) | Sub2-2(55)-4 | m/z = 322.01(C₁₇H₁₁BrN₂ = 323.19) |
| Sub2-2(56)-4 | m/z = 322.01(C₁₇H₁₁BrN₂ = 323.19) | Sub2-2(57)-4 | m/z = 322.01(C₁₇H₁₁BrN₂ = 323.19) |
| Sub2-3(1)-4 | m/z = 322.01(C₁₇H₁₁BrN₂ = 323.19) | Sub2-3(2)-4 | m/z = 398.04(C₂₃H₁₅BrN₂ = 399.28) |
| Sub2-3(3)-4 | m/z = 398.04(C₂₃H₁₅BrN₂ = 389.28) | Sub2-3(4)-4 | m/z = 477.06(C₂₆H₁₆BrN₅ = 478.34) |
| Sub2-3(5)-4 | m/z = 475.07(C₂₈H₁₈BrN₃ = 476.37) | Sub2-3(6)-4 | m/z = 475.07(C₂₈H₁₈BrN₃ = 476.37) |
| Sub2-3(7)-4 | m/z = 476.06(C₂₈H₁₈BrN₃ = 477.35) | Sub2-3(8)-4 | m/z = 476.06(C₂₈H₁₈BrN₃ = 477.35) |
| Sub2-3(9)-4 | m/z = 476.06(C₂₈H₁₈BrN₃ = 477.35) | Sub2-3(10)-4 | m/z = 476.06(C₂₈H₁₈BrN₃ = 477.35) |
| Sub2-3(11)-4 | m/z = 477.06(C₂₈H₁₈BrN₃ = 478.34) | Sub2-3(12)-4 | m/z = 551.10(C₃₄H₂₂BrN₃ = 552.46) |
| Sub2-3(13)-4 | m/z = 551.10(C₃₄H₂₂BrN₃ = 552.46) | Sub2-3(14)-4 | m/z = 552.09(C₃₃H₂₁BrN₄ = 553.45) |
| Sub2-3(15)-4 | m/z = 553.09(C₃₂H₂₀BrN₅ = 554.44) | Sub2-3(16)-4 | m/z = 553.09(C₃₂H₂₀BrN₅ = 554.44) |
| Sub2-3(17)-4 | m/z = 552.09(C₃₃H₂₁BrN₄ = 553.45) | Sub2-3(18)-4 | m/z = 552.09(C₃₃H₂₁BrN₄ = 553.45) |
| Sub2-3(19)-4 | m/z = 553.09(C₃₂H₂₀BrN₅ = 554.44) | Sub2-3(20)-4 | m/z = 552.09(C₃₃H₂₁BrN₄ = 553.45) |
| Sub2-3(21)-4 | m/z = 552.09(C₃₃H₂₁BrN₄ = 553.45) | Sub2-3(22)-4 | m/z = 551.10(C₃₄H₂₇BrN₃ = 552.46) |
| Sub2-3(23)-4 | m/z = 551.10(C₃₄H₂₇BrN₃ = 552.46) | Sub2-3(24)-4 | m/z = 552.09(C₃₃H₂₁BrN₄ = 553.45) |
| Sub2-3(25)-4 | m/z = 553.09(C₃₂H₂₀BrN₅ = 554.44) | Sub2-3(26)-4 | m/z = 450.05(C₂₅H₁₅BrN₄ = 451.32) |
| Sub2-3(27)-4 | m/z = 322.01(C₁₇H₁₁BrN₂ = 323.19) | Sub2-3(28)-4 | m/z = 322.01(C₁₇H₁₁BrN₂ = 323.19) |
| Sub2-3(29)-4 | m/z = 322.01(C₁₇H₁₁BrN₂ = 323.19) | Sub2-3(30)-4 | m/z = 398.04(C₂₈H₁₅BrN₂ = 399.28) |
| Sub2-3(31)-4 | m/z = 398.04(C₂₈H₁₅BrN₂ = 399.28) | Sub2-3(32)-4 | m/z = 450.05(C₂₅H₁₅BrN₄ = 451.32) |
| Sub2-3(33)-4 | m/z = 475.07(C₂₈H₁₈BrN₃ = 476.37) | Sub2-3(34)-4 | m/z = 475.07(C₂₈H₁₈BrN₃ = 476.37) |
| Sub2-3(35)-4 | m/z = 476.06(C₂₈H₁₈BrN₃ = 477.35) | Sub2-3(36)-4 | m/z = 476.06(C₂₈H₁₈BrN₃ = 477.35) |
| Sub2-3(37)-4 | m/z = 476.06(C₂₈H₁₈BrN₃ = 477.35) | Sub2-3(38)-4 | m/z = 476.06(C₂₈H₁₈BrN₃ = 477.35) |
| Sub2-3(39)-4 | m/z = 477.06(C₂₆H₁₈BrN₅ = 478.34) | Sub2-3(40)-4 | m/z = 477.06(C₂₆H₁₈BrN₅ = 478.34) |
| Sub2-3(41)-4 | m/z = 551.10(C₃₄H₂₂BrN₃ = 552.46) | Sub2-3(42)-4 | m/z = 551.10(C₃₄H₂₂BrN₃ = 552.46) |
| Sub2-3(43)-4 | m/z = 552.09(C₃₃H₂₁BrN₄ = 553.45) | Sub2-3(44)-4 | m/z = 553.09(C₃₂H₂₀BrN₅ = 554.44) |
| Sub2-3(45)-4 | m/z = 553.09(C₃₂H₂₀BrN₅ = 554.44) | Sub2-3(46)-4 | m/z = 552.09(C₃₃H₂₁BrN₄ = 553.45) |
| Sub2-3(47)-4 | m/z = 552.09(C₃₃H₂₁BrN₄ = 553.45) | Sub2-3(48)-4 | m/z = 553.09(C₃₂H₂₀BrN₅ = 554.44) |
| Sub2-3(49)-4 | m/z = 551.10(C34H₂₂BrN₃ = 552.46) | Sub2-3(50)-4 | m/z = 551.10(C₃₄H₂₂BrN₃ = 552.46) |
| Sub2-3(51)-4 | m/z = 552.09(C₃₃H₂₁BrN₄ = 553.45) | Sub2-3(52)-4 | m/z = 553.09(C₃₂H₂₀BrN₅ = 554.44) |
| Sub2-3(53)-4 | m/z = 553.09(C₃₂H₂₀BrN₅ = 554.44) | Sub2-3(54)-4 | m/z = 552.09(C₃₃H₂₁BrN₄ = 553.45) |
| Sub2-3(55)-4 | m/z = 553.09(C₃₂H₂₀BrN₃ = 554.44) | Sub2-4(1)-4 | m/z = 322.01(C₁₇H₁₁BrN₂ = 323.19) |
| Sub2-4(2)-4 | m/z = 322.01(C₁₇H₁₁BrN₂ = 323.19) | Sub2-4(3)-4 | m/z = 322.01(C₁₇H₁₁BrN₂ = 323.19) |
| Sub2-4(4)-4 | m/z = 322.01(C₁₇H₁₁BrN₂ = 323.19) | Sub2-4(5)-4 | m/z = 322.01(C₁₇H₁₁BrN₂ = 323.19) |
| Sub2-4(6)-4 | m/z = 322.01(C₁₇H₁₁BrN₂ = 323.19) | Sub2-4(7)-4 | m/z = 322.01(C₁₇H₁₁BrN₂ = 323.19) |

### III. Synthesis Example of Final Products

### Synthesis Example of Compound 1-1-4

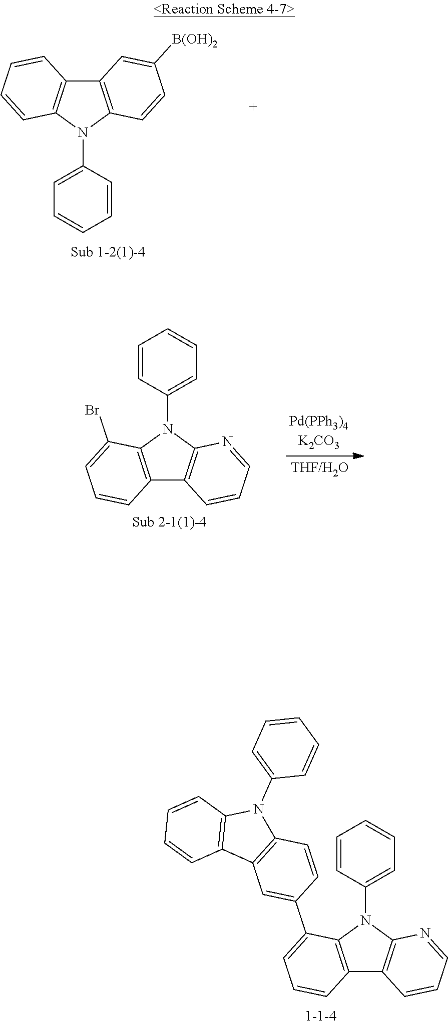 [SMILES: OB(O)c1ccc2c(c1)c1ccccc1n2-c1ccccc1 ; Brc1cccc2c3cccnc3n(-c3ccccc3)c12 ; c1ccc(-n2c3ccccc3c3cc(-c4cccc5c6cccnc6n(-c6ccccc6)c45)ccc32)cc1]

In a round-bottom flask, (9-phenyl-9H-carbazol-3-yl)boronic acid (5.7 g, 20 mmol) was added, and then 8-bromo-9-phenyl-9H-pyrido[2,3-b]indole (12.2 g, 22 mmol), Pd(PPh₃)₄ (0.03-0.05 eq), K₂CO₃ (3 eq), THF (10 mL), and water (5 mL) were added. Thereafter, the mixture was heated under reflux at 80-90° C. Upon completion of the reaction, the reaction product was diluted with distilled water at room temperature, followed by extraction with methylene chloride and water. The organic layer was dried over MgSO₄ and concentrated, and then the thus generated compound was subjected to silica gel column chromatography and recrystallization to give a product 5.5 g (yield: 57%).

### 2. Synthesis Example of Compound 2-38-4

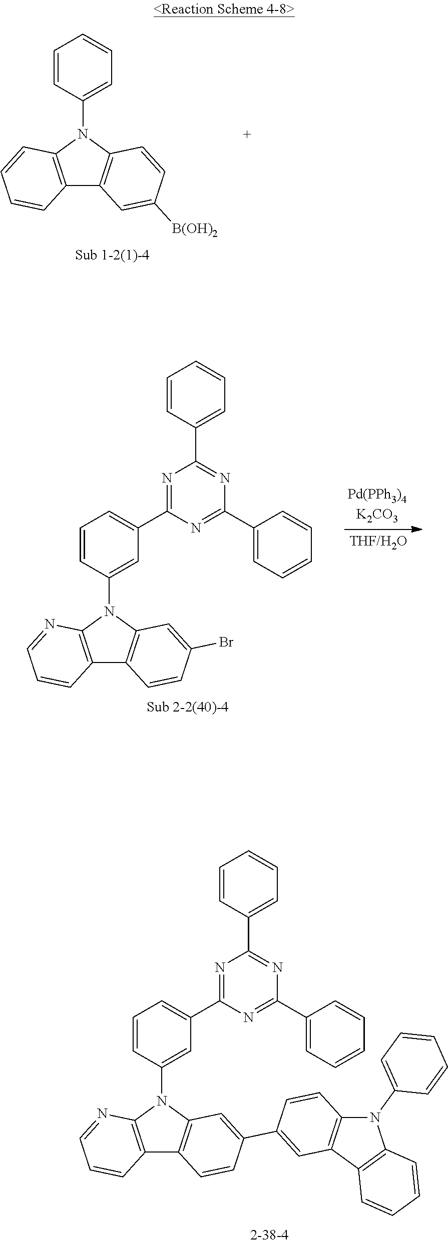 [SMILES: OB(O)c1ccc2c(c1)c1ccccc1n2-c1ccccc1 ; Brc1ccc2c3cccnc3n(-c3cccc(-c4nc(-c5ccccc5)nc(-c5ccccc5)n4)c3)c2c1 ; c1ccc(-c2nc(-c3ccccc3)nc(-c3cccc(-n4c5cc(-c6ccc7c(c6)c6ccccc6n7-c6ccccc6)ccc5c5cccnc54)c3)n2)cc1]

In a round-bottom flask, (9-phenyl-9H-carbazol-3-yl)boronic acid (5.7 g, 20 mmol) was added, and then 7-bromo-9-(3-(4,6-diphenyl-1,3,5-triazin-2-yl)phenyl)-9H-pyrido[2,3-b]indole (12.2 g, 22 mmol), Pd(PPh₃)₄ (0.03-0.05 eq), K₂CO₃ (3 eq), THF (10 mL), and water (5 mL) were added. Thereafter, the mixture was heated under reflux at 80-90° C. Upon completion of the reaction, the reaction product was diluted with distilled water at room temperature, followed by extraction with methylene chloride and water. The organic layer was dried over MgSO₄ and concentrated, and then the thus generated compound was subjected to silica gel column chromatography and recrystallization to give a product 8.2 g (yield: 57%).

### 3. Synthesis Example of Compound 2-70-4

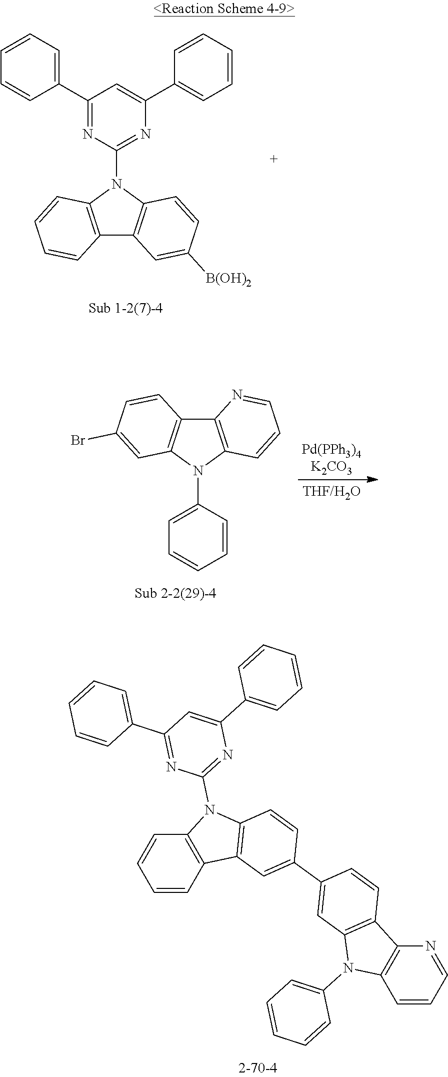 [SMILES: OB(O)c1ccc2c(c1)c1ccccc1n2-c1nc(-c2ccccc2)cc(-c2ccccc2)n1 ; "+" ; Brc1ccc2c3ncccc3n(-c3ccccc3)c2c1 ; c1ccc(-c2cc(-c3ccccc3)nc(-n3c4ccccc4c4cc(-c5ccc6c7ncccc7n(-c7ccccc7)c6c5)ccc43)n2)cc1]

In a round-bottom flask, (9-(4,6-diphenylpyrimidin-2-yl)-9H-carbazol-3-yl)boronic acid (8.8 g, 20 mmol) was added, and then 7-bromo-9-(3-(4,6-diphenyl-1,3,5-triazin-2-yl)phenyl)-9H-pyrido[2,3-b]indole (12.2 g, 22 mmol), Pd(PPh₃)₄ (0.03-0.05 eq), K₂CO₃ (3 eq), THF (10 mL), and water (5 mL) were added. Thereafter, the mixture was heated under reflux at 80-90° C. Upon completion of the reaction, the reaction product was diluted with distilled water at room temperature, followed by extraction with methylene chloride and water. The organic layer was dried over MgSO₄ and concentrated, and then the thus generated compound was subjected to silica gel column chromatography and recrystallization to give a product 8.0 g (yield: 62%).

### 4. Synthesis Example of Compound 3-10-4

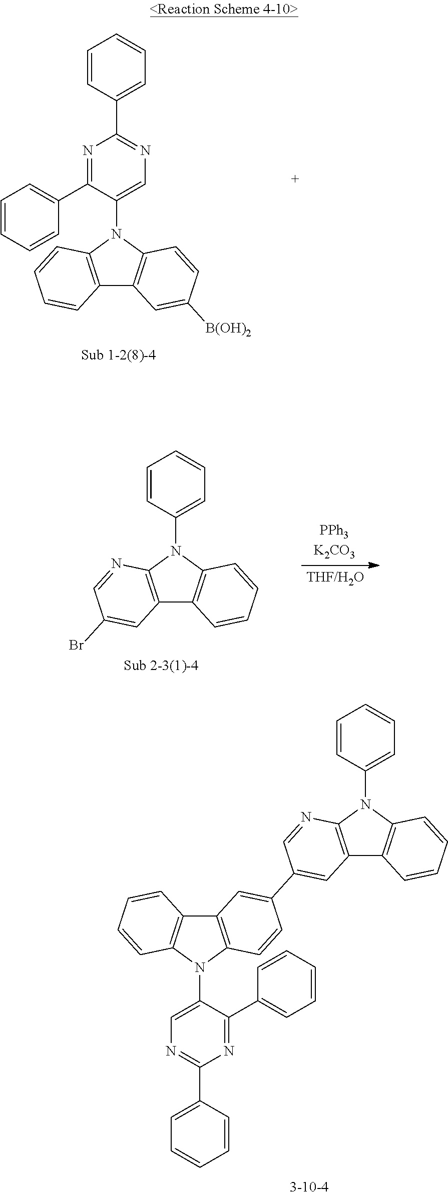 [SMILES: OB(O)c1ccc2c(c1)c1ccccc1n2-c1cnc(-c2ccccc2)nc1-c1ccccc1 ; Brc1cnc2c(c1)c1ccccc1n2-c1ccccc1 ; c1ccc(-c2ncc(-n3c4ccccc4c4cc(-c5cnc6c(c5)c5ccccc5n6-c5ccccc5)ccc43)c(-c3ccccc3)n2)cc1]

In a round-bottom flask, (9-(2,4-diphenylpyrimidin-5-yl)-9H-carbazol-3-yl)boronic acid (8.8 g, 20 mmol) was added, and then 6-bromo-9-phenyl-9H-pyrido[2,3-b]indole (7.1 g, 22 mmol), Pd(PPh₃)₄ (0.03-0.05 eq), K₂CO₃ (3 eq), THF (10 mL), and water (5 mL) were added. Thereafter, the mixture was heated under reflux at 80-90° C. Upon completion of the reaction, the reaction product was diluted with distilled water at room temperature, followed by extraction with methylene chloride and water. The organic layer was dried over MgSO₄ and concentrated, and then the thus generated compound was subjected to silica gel column chromatography and recrystallization to give a product 7.3 g (yield: 57%).

### 5. Synthesis Example of Compound 3-68-4

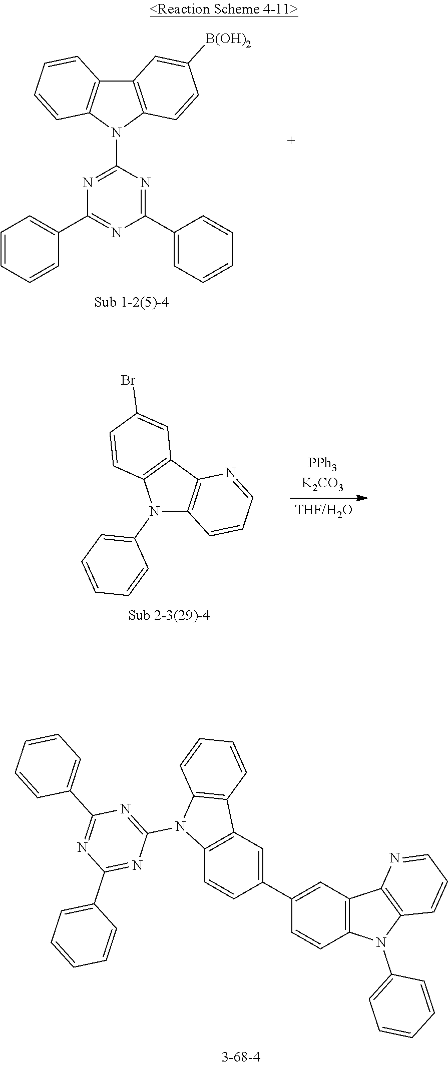 [SMILES: OB(O)c1ccc2c(c1)c1ccccc1n2-c1nc(-c2ccccc2)nc(-c2ccccc2)n1 ; Brc1ccc2c(c1)c1ncccc1n2-c1ccccc1 ; c1ccc(-c2nc(-c3ccccc3)nc(-n3c4ccccc4c4cc(-c5ccc6c(c5)c5ncccc5n6-c5ccccc5)ccc43)n2)cc1]

In a round-bottom flask, (9-(4,6-diphenyl-1,3,5-triazin-2-yl)-9H-carbazol-3-yl)boronic acid (8.8 g, 20 mmol) was added, and then 8-bromo-5-phenyl-5H-pyrido[3,2-b]indole (7.1 g, 22 mmol), Pd(PPh₃)₄ (0.03-0.05 eq), K₂CO₃ (3 eq), THF (10 mL), and water (5 mL) were added. Thereafter, the mixture was heated under reflux at 80-90° C. Upon completion of the reaction, the reaction product was diluted with distilled water at room temperature, followed by extraction with methylene chloride and water. The organic layer was dried over MgSO₄ and concentrated, and then the thus generated compound was subjected to silica gel column chromatography and recrystallization to give a product 7.0 g (yield: 54%).

### 6. Synthesis Example of Compound 3-76-4

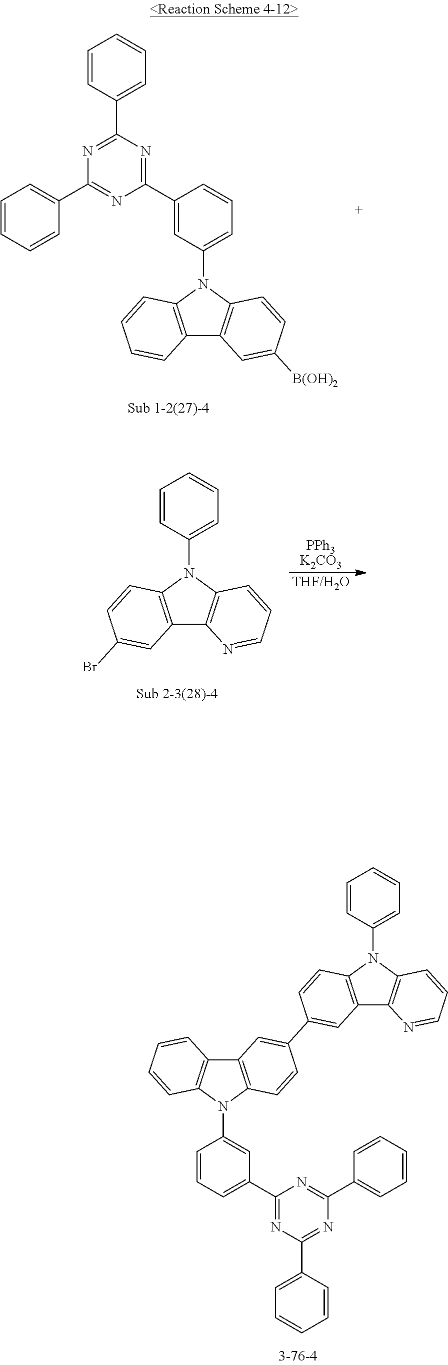 [SMILES: OB(O)c1ccc2c(c1)c1ccccc1n2-c1cccc(-c2nc(-c3ccccc3)nc(-c3ccccc3)n2)c1 ; "+" ; Brc1ccc2c(c1)c1ncccc1n2-c1ccccc1 ; "PPh3K2CO3" ; "THF/H2O" ; c1ccc(-c2nc(-c3ccccc3)nc(-c3cccc(-n4c5ccccc5c5cc(-c6ccc7c(c6)c6ncccc6n7-c6ccccc6)ccc54)c3)n2)cc1]

In a round-bottom flask, (9-(3-(4,6-diphenyl-1,3,5-triazin-2-yl)phenyl)-9H-carbazol-3-yl)boronic acid (10.4 g, 20 mmol) was added, and then 8-bromo-5-phenyl-5H-pyrido[3,2-b]indole (7.1 g, 22 mmol), Pd(PPh₃)₄ (0.03-0.05 eq), K₂CO₃ (3 eq), THF (10 mL), and water (5 mL) were added. Thereafter, the mixture was heated under reflux at 80-90° C. Upon completion of the reaction, the reaction product was diluted with distilled water at room temperature, followed by extraction with methylene chloride and water. The organic layer was dried over MgSO₄ and concentrated, and then the thus generated compound was subjected to silica gel column chromatography and recrystallization to give a product 10.5 g (yield: 73%).

### 7. Synthesis Example of Compound 4-23-4

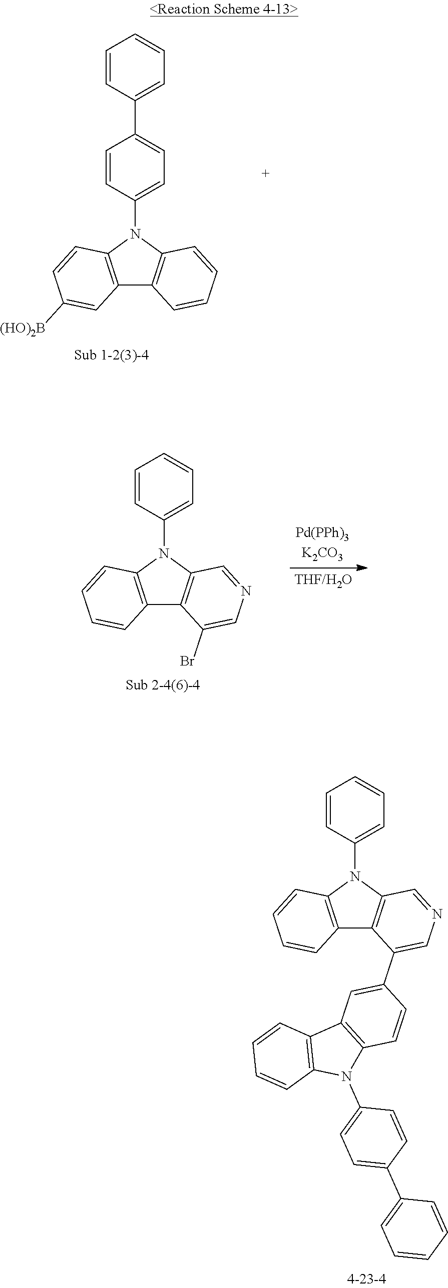 [SMILES: OB(O)c1ccc2c(c1)c1ccccc1n2-c1ccc(-c2ccccc2)cc1 ; Brc1cncc2c1c1ccccc1n2-c1ccccc1 ; c1ccc(-c2ccc(-n3c4ccccc4c4cc(-c5cncc6c5c5ccccc5n6-c5ccccc5)ccc43)cc2)cc1]

In a round-bottom flask, (9-([1,1′-biphenyl]-4-yl)-9H-carbazol-3-yl)boronic acid (7.2 g, 20 mmol) was added, and then 4-bromo-9-phenyl-9H-pyrido[3,4-b]indole (7.1 g, 22 mmol), Pd(PPh₃)₄ (0.03-0.05 eq), K₂CO₃ (3 eq), THF (10 mL), and water (5 mL) were added. Thereafter, the mixture was heated under reflux at 80-90° C. Upon completion of the reaction, the reaction product was diluted with distilled water at room temperature, followed by extraction with methylene chloride and water. The organic layer was dried over MgSO₄ and concentrated, and then the thus generated compound was subjected to silica gel column chromatography and recrystallization to give a product 7.8 g (yield: 69%).

Meanwhile, FD-MS values of compounds 1-1-4 to 1-28-4, 2-1-4 to 2-128-4, 3-1-4 to 3-127-4, 4-1-4 to 4-28-4, and 5-1-4 to 5-4-4 of the present invention prepared by the above synthesis examples are shown as in table 4-4 below.

TABLE 4-4

| Compound | FD-MS | Compound | FD-MS |
|---|---|---|---|
| 1-1-4 | m/z = 485.19 (C₃₅H₂₃N₃ = 485.58) | 1-2-4 | m/z = 535.20 (C₃₉H₂₅N₃ = 535.64) |
| 1-3-4 | m/z = 561.22 (C₄₁H₂₇N₃ = 561.67) | 1-4-4 | m/z = 640.24 (C₄₄H₂₈N₆ = 640.73) |
| 1-5-4 | m/z = 485.19 (C₃₅H₂₃N₃ = 485.58) | 1-6-4 | m/z = 535.20 (C₃₉H₂₅N₃ = 535.64) |
| 1-7-4 | m/z = 561.22 (C₄₁H₂₇N₃ = 561.67) | 1-8-4 | m/z = 640.24 (C₄₄H₂₈N₆ = 640.73) |
| 1-9-4 | m/z = 485.19 (C₃₅H₂₃N₃ = 485.58) | 1-10-4 | m/z = 535.20 (C₃₉H₂₅N₃ = 535.64) |
| 1-11-4 | m/z = 561.22 (C₄₁H₂₇N₃ = 561.67) | 1-12-4 | m/z = 640.24 (C₄₄H₂₈N₆ = 640.73) |
| 1-13-4 | m/z = 485.19 (C₃₅H₂₃N₃ = 485.5.8) | 1-14-4 | m/z = 535.20 (C₃₉H₂₅N₃ = 535.64) |
| 1-15-4 | m/z = 561.22 (C₄₁H₂₂N₃ = 561.67) | 1-16-4 | m/z = 640.24 (C₄₄H₂₈N₆ = 640.73) |
| 1-17-4 | m/z = 485.19 (C₃₅H₂₃N₃ = 485.58) | 1-18-4 | m/z = 535.20 (C₃₉H₂₅N₃ = 535.64) |
| 1-19-4 | m/z = 561.22 (C₄₁H₂₇N₃ = 561.67) | 1-20-4 | m/z = 640.24 (C₄₄H₂₈N₆ = 640.73) |
| 1-21-4 | m/z = 485.19 (C₃₅H₂₃N₃ = 485.58) | 1-22-4 | m/z = 535.20 (C₃₉H₂₅N₃ = 535.64) |
| 1-23-4 | m/z = 561.22 (C₄₁H₂₇N₃ = 561.67) | 1-24-4 | m/z = 640.24 (C₄₄H₂₈N₆ = 640.73) |
| 1-25-4 | m/z = 485.19 (C₃₅H₂₃N₃ = 485.58) | 1-26-4 | m/z = 535.20 (C₃₉H₂₅N₃ = 535.64) |
| 1-27-4 | m/z = 561.22 (C₄₁H₂₇N₃ = 561.67) | 1-28-4 | m/z = 640.24 (C₄₄H₂₈N₆ = 640.73) |
| 2-1-4 | m/z = 485.19 (C₃₅H₂₃N₃ = 485.5.8) | 2-2-4 | m/z = 561.22 (C₄₁H₂₇N₃ = 561.67) |
| 2-3-4 | m/z = 561.22 (C₄₁H₂₂N₃ = 561.67) | 2-4-4 | m/z = 637.25 (C₄₇H₃₁N₃ = 637.77) |
| 2-5-4 | m/z = 637.25 (C₄₇H₃₁N₃ = 637.77) | 2-6-4 | m/z = 637.25 (C₄₇H₃₁N₃ = 637.77) |
| 2-7-4 | m/z = 637.25 (C₄₇H₃₁N₃ = 637.77) | 2-8-4 | m/z = 639.24 (C₄₅H₂₉N₅ = 639.75) |
| 2-9-4 | m/z = 639.24 (C₄₅H₂₉N₅ = 639.75) | 2-10-4 | m/z = 639.24 (C₄₅H₂₉N₅ = 639.75) |
| 2-11-4 | m/z = 638.25 (C₄₆H₃₀N₄ = 638.76) | 2-12-4 | m/z = 638.25 (C₄₆H₃₀N₄ = 638.76) |
| 2-13-4 | m/z = 639.24 (C₄₅H₂₉N₅ = 639.75) | 2-14-4 | m/z = 640.24 (C₄₄H₂₈N₆ = 640.73) |
| 2-15-4 | m/z = 716.27 (C₃₈H₃₂N₆ = 716.83) | 2-16-4 | m/z = 715.27 (C₅₁H₃₃N₅ = 715.84) |
| 2-17-4 | m/z = 715.27 (C₅₁H₃₃N₅ = 715.84) | 2-18-4 | m/z = 714.28 (C₅₂H₃₄N₄ = 714.85) |
| 2-19-4 | m/z = 714.28 (C₅₂H₃₄N₄ = 714.85) | 2-20-4 | m/z = 715.27 (C₅₁H₃₃N₅ = 715.84) |
| 2-21-4 | m/z = 716.27 (C₅₀H₃₂N₆ = 716.83) | 2-22-4 | m/z = 716.27 (C₃₈H₃₂N₆ = 716.83) |
| 2-23-4 | m/z = 715.27 (C₅₁H₃₃N₅ = 715.84) | 2-24-4 | m/z = 715.27 (C₅₁H₃₃N₅ = 715.84) |
| 2-25-4 | m/z = 714.28 (C₅₂H₃₄N₄ = 714.85) | 2-26-4 | m/z = 714.28 (C₅₂H₃₄N₄ = 714.85) |
| 2-27-4 | m/z = 715.27 (C₅₁H₃₃N₅ = 715.84) | 2-28-4 | m/z = 716.27 (C₃₈H₃₂N₆ = 716.83) |
| 2-29-4 | m/z = 613.23 (C₄₃H₂₇N₅ = 613.71) | 2-30-4 | m/z = 640.24 (C₄₄H₂₈N₆ = 640.73) |
| 2-31-4 | m/z = 639.24 (C₄₅H₂₉N₅ = 639.75) | 2-32-4 | m/z = 639.24 (C₄₅H₂₉N₅ = 639.75) |
| 2-33-4 | m/z = 639.24 (C₄₅H₂₉N₅ = 639.75) | 2-34-4 | m/z = 638.25 (C₄₆H₃₀N₄ = 638.76) |
| 2-35-4 | m/z = 638.25 (C₄₆H₃₀N₄ = 638.76) | 2-36-4 | m/z = 639.24 (C₄₅H₂₉N₅ = 639.75) |
| 2-37-4 | m/z = 640.24 (C₄₄H₂₈N₆ = 640.73) | 2-38-4 | m/z = 716.27 (C₃₈H₃₂N₆ = 716.83) |
| 2-39-4 | m/z = 715.27 (C₅₁H₃₃N₅ = 715.84) | 2-40-4 | m/z = 715.27 (C₅₁H₃₃N₅ = 715.84) |
| 2-41-4 | m/z = 714.28 (C₅₂H₃₄N₄ = 714.85) | 2-42-4 | m/z = 714.28 (C₅₂H₃₄N₄ = 714.85) |
| 2-43-4 | m/z = 715.27 (C₅₁H₃₃N₅ = 715.84) | 2-44-4 | m/z = 716.27 (C₅₀H₃₂N₆ = 716.83) |
| 2-45-4 | m/z = 716.27 (C₅₀H₃₂N₆ = 716.83) | 2-46-4 | m/z = 715.27 (C₅₁H₃₃N₅ = 715.84) |
| 2-47-4 | m/z = 715.27 (C₅₁H₃₃N₅ = 715.84) | 2-48-4 | m/z = 714.28 (C₅₂H₃₄N₄ = 714.85) |
| 2-49-4 | m/z = 714.28 (C₅₂H₃₄N₄ = 714.85) | 2-50-4 | m/z = 715.27 (C₅₁H₃₃N₅ = 715.84) |
| 2-51-4 | m/z = 716.27 (C₅₀H₃₂N₆ = 716.83) | 2-52-4 | m/z = 613.23 (C₄₃H₂₇N₅ = 613.71) |
| 2-53-4 | m/z = 485.19 (C₃₅H₂₃N₃ = 485.58) | 2-54-4 | m/z = 535.20 (C₃₉H₂₅N₃ = 535.64) |
| 2-55-4 | m/z = 561.22 (C₄₁H₂₂N₃ = 561.67) | 2-56-4 | m/z = 640.24 (C₄₄H₂₈N₆ = 640.73) |
| 2-57-4 | m/z = 485.19 (C₃₅H₂₃N₃ = 485.58) | 2-58-4 | m/z = 535.20 (C₃₉H₂₅N₃ = 535.64) |
| 2-59-4 | m/z = 561.22 (C₄₁H₂₂N₃ = 561.67) | 2-60-4 | m/z = 640.24 (C₄₄H₂₈N₆ = 640.73) |
| 2-61-4 | m/z = 485.19 (C₃₅H₂₃N₃ = 485.58) | 2-62-4 | m/z = 561.22 (C₄₁H₂₂N₃ = 561.67) |
| 2-63-4 | m/z = 561.22 (C₄₁H₂₂N₃ = 561.67) | 2-64-4 | m/z = 637.25 (C₄₇H₃₁N₃ = 637.77) |
| 2-65-4 | m/z = 637.25 (C₄₇H₃₁N₃ = 637.77) | 2-66-4 | m/z = 637.25 (C₄₇H₃₁N₃ = 637.77) |
| 2-67-4 | m/z = 637.25 (C₄₇H₃₁N₃ = 637.77) | 2-68-4 | m/z = 640.24 (C₄₄H₂₈N₆ = 640.73) |
| 2-69-4 | m/z = 639.24 (C₄₅H₂₉N₅ = 639.75) | 2-70-4 | m/z = 639.24 (C₄₅H₂₉N₅ = 639.75) |
| 2-71-4 | m/z = 639.24 (C₄₅H₂₉N₅ = 639.75) | 2-72-4 | m/z = 638.25 (C₄₆H₃₀N₄ = 638.76) |
| 2-73-4 | m/z = 638.25 (C₄₆H₃₀N₄ = 638.76) | 2-74-4 | m/z = 639.24 (C₄₅H₂₉N₅ = 639.75) |
| 2-75-4 | m/z = 640.24 (C₄₄H₂₈N₆ = 640.73) | 2-76-4 | m/z = 716.27 (C₅₀H₃₂N₆ = 716.83) |
| 2-77-4 | m/z = 715.27 (C₅₁H₃₃N₅ = 715.84) | 2-78-4 | m/z = 715.27 (C₅₁H₃₃N₅ = 715.84) |
| 2-79-4 | m/z = 714.28 (C₅₂H₃₄N₄ = 714.85) | 2-80-4 | m/z = 714.28 (C₅₂H₃₄N₄ = 714.85) |
| 2-81-4 | m/z = 715.27 (C₅₁H₃₃N₅ = 715.84) | 2-82-4 | m/z = 716.27 (C₅₀H₃₂N₆ = 716.83) |
| 2-83-4 | m/z = 716.27 (C₅₀H₃₂N₆ = 716.83) | 2-84-4 | m/z = 715.27 (C₅₁H₃₃N₅ = 715.84) |
| 2-85-4 | m/z = 715.27 (C₅₁H₃₃N₅ = 715.84) | 2-86-4 | m/z = 714.28 (C₅₂H₃₄N₄ = 714.85) |
| 2-87-4 | m/z = 714.28 (C₅₂H₃₄N₄ = 714.85) | 2-88-4 | m/z = 715.27 (C₅₁H₃₃N₅ = 715.84) |
| 2-89-4 | m/z = 716.27 (C₅₀H₃₂N₆ = 716.83) | 2-90-4 | m/z = 613.23 (C₄₃H₂₇N₅ = 613.71) |
| 2-91-4 | m/z = 640.24 (C₄₄H₂₈N₆ = 640.73) | 2-92-4 | m/z = 639.24 (C₄₅H₂₉N₅ = 639.75) |
| 2-93-4 | m/z = 639.24 (C₄₅H₂₉N₅ = 639.75) | 2-94-4 | m/z = 639.24 (C₄₅H₂₉N₅ = 639.75) |
| 2-95-4 | m/z = 638.25 (C₄₆H₃₀N₄ = 638.76) | 2-96-4 | m/z = 638.25 (C₄₆H₃₀N₄ = 638.76) |
| 2-97-4 | m/z = 639.24 (C₄₅H₂₉N₅ = 639.75) | 2-98-4 | m/z = 640.24 (C₄₄H₂₈N₆ = 640.73) |
| 2-99-4 | m/z = 716.27 (C₅₀H₃₂N₆ = 716.83) | 2-100-4 | m/z = 715.27 (C₅₁H₃₃N₅ = 715.84) |
| 2-101-4 | m/z = 715.27 (C₅₁H₃₃N₅ = 715.84) | 2-102-4 | m/z = 714.28 (C₅₂H₃₄N₄ = 714.85) |
| 2-103-4 | m/z = 714.28 (C₅₂H₃₄N₄ = 714.85) | 2-104-4 | m/z = 715.27 (C₅₁H₃₃N₅ = 715.84) |
| 2-105-4 | m/z = 716.27 (C₅₀H₃₂N₆ = 716.83) | 2-106-4 | m/z = 716.27 (C₅₀H₃₂N₆ = 716.83 |
| 2-107-4 | m/z = 715.27 (C₅₁H₃₃N₅ = 715.84) | 2-108-4 | m/z = 715.27 (C₅₁H₃₃N₅ = 715.84) |
| 2-109-4 | m/z = 714.28 (C₅₂H₃₄N₄ = 714.85) | 2-110-4 | m/z = 714.28 (C₅₂H₃₄N₄ = 714.85) |
| 2-111-4 | m/z = 715.27 (C₅₁H₃₃N₅ = 715.84) | 2-112-4 | m/z = 716.27 (C₅₀H₃₂N₆ = 716.83) |
| 2-113-4 | m/z = 613.23 (C₄₃H₂₇N₅ = 613.71) | 2-114-4 | m/z = 485.19 (C₃₅H₂₃N₃ = 485.58) |
| 2-115-4 | m/z = 535.20 (C₃₉H₂₅N₃ = 535.64) | 2-116-4 | m/z = 561.22 (C₄₁H₂₂N₃ = 561.67) |
| 2-117-4 | m/z = 640.24 (C₄₄H₂₈N₆ = 640.73) | 2-118-4 | m/z = 485.19 (C₃₅H₂₃N₃ = 485.58) |
| 2-119-4 | m/z = 535.20 (C₃₉H₂₅N₃ = 535.64) | 2-120-4 | m/z = 561.22 (C₄₁H₂₂N₃ = 561.67) |
| 2-121-4 | m/z = 640.24 (C₄₄H₂₈N₆ = 640.73) | 2-122-4 | m/z = 485.19 (C₃₅H₂₃N₃ = 485.58) |
| 2-123-4 | m/z = 535.20 (C₃₉H₂₅N₃ = 535.64) | 2-124-4 | m/z = 561.22 (C₄₁H₂₂N₃ = 561.67) |
| 2-125-4 | m/z = 640.24 (C₄₄H₂₈N₆ = 640.73) | 2-126-4 | m/z = 640.24 (C₄₄H₂₈N₆ = 640.73) |
| 2-127-4 | m/z = 535.20 (C₃₉H₂₅N₃ = 535.64) | 2-128-4 | m/z = 535.20 (C₃₉H₂₅N₃ = 535.64) |
| 3-1-4 | m/z = 485.19 (C₃₅H₂₃N₃ = 485.58) | 3-2-4 | m/z = 561.22 (C₄₁H₂₂N₃ = 561.67) |
| 3-3-4 | m/z = 561.22 (C₄₁H₂₂N₃ = 561.67) | 3-4-4 | m/z = 637.25 (C₄₇H₃₁N₃ = 637.77) |
| 3-5-4 | m/z = 637.25 (C₄₇H₃₁N₃ = 637.77) | 3-6-4 | m/z = 637.25 (C₄₇H₃₁N₃ = 637.77) |
| 3-7-4 | m/z = 637.25 (C₄₇H₃₁N₃ = 637.77) | 3-8-4 | m/z = 639.24 (C₄₅H₂₉N₅ = 639.75) |
| 3-9-4 | m/z = 639.24 (C₄₅H₂₉N₅ = 639.75) | 3-10-4 | m/z = 639.24 (C₄₅H₂₉N₅ = 639.75) |
| 3-11-4 | m/z = 638.25 (C₄₆H₃₀N₄ = 638.76) | 3-12-4 | m/z = 638.25 (C₄₆H₃₀N₄ = 638.76) |
| 3-13-4 | m/z = 639.24 (C₄₅H₂₉N₅ = 639.75) | 3-14-4 | m/z = 640.24 (C₄₄H₂₈N₆ = 640.73) |
| 3-15-4 | m/z = 716.27 (C₅₀H₃₂N₆ = 716.83) | 3-16-4 | m/z = 715.27 (C₅₁H₃₃N₅ = 715.84) |
| 3-17-4 | m/z = 715.27 (C₅₁H₃₃N₅ = 715.84) | 3-18-4 | m/z = 714.28 (C₅₂H₃₄N₄ = 714.85) |
| 3-19-4 | m/z = 714.28 (C₅₂H₃₄N₄ = 714.85) | 3-20-4 | m/z = 715.27 (C₅₁H₃₃N₅ = 715.84) |
| 3-21-4 | m/z = 716.27 (C₅₀H₃₂N₆ = 716.83) | 3-22-4 | m/z = 716.27 (C₅₀H₃₂N₆ = 716.83) |
| 3-23-4 | m/z = 715.27 (C₅₁H₃₃N₅ = 715.84) | 3-24-4 | m/z = 715.27 (C₅₁H₃₃N₅ = 715.84) |
| 3-25-4 | m/z = 714.28 (C₅₂H₃₄N₄ = 714.85) | 3-26-4 | m/z = 714.28 (C₅₂H₃₄N₄ = 714.85) |
| 3-27-4 | m/z = 715.27 (C₅₁H₃₃N₅ = 715.84) | 3-28-4 | m/z = 716.27 (C₅₀H₃₂N₆ = 716.83) |
| 3-29-4 | m/z = 613.23 (C₄₃H₂₇N₅ = 613.71) | 3-30-4 | m/z = 640.24 (C₄₄H₂₈N₆ = 640.73) |
| 3-31-4 | m/z = 639.24 (C₄₅H₂₉N₅ = 639.75) | 3-32-4 | m/z = 639.24 (C₄₅H₂₉N₅ = 639.75) |
| 3-33-4 | m/z = 639.24 (C₄₅H₂₉N₅ = 639.75) | 3-34-4 | m/z = 638.25 (C₄₆H₃₀N₄ = 638.76) |
| 3-35-4 | m/z = 638.25 (C₄₆H₃₀N₄ = 638.76) | 3-36-4 | m/z = 639.24 (C₄₅H₂₉N₅ = 639.75) |
| 3-37-4 | m/z = 640.24 (C₄₄H₂₈N₆ = 640.73) | 3-38-4 | m/z = 716.27 (C₅₀H₃₂N₆ = 716.83) |
| 3-39-4 | m/z = 715.27 (C₅₁H₃₃N₅ = 715.84) | 3-40-4 | m/z = 715.27 (C₅₁H₃₃N₅ = 715.84) |
| 3-41-4 | m/z = 714.28 (C₅₂H₃₄N₄ = 714.85) | 3-42-4 | m/z = 714.28 (C₅₂H₃₄N₄ = 714.85) |
| 3-43-4 | m/z = 715.27 (C₅₁H₃₃N₅ = 715.84) | 3-44-4 | m/z = 716.27 (C₅₀H₃₂N₆ = 716.83) |
| 3-45-4 | m/z = 716.27 (C₅₀H₃₂N₆ = 716.83) | 3-46-4 | m/z = 715.27 (C₅₁H₃₃N₅ = 715.84) |
| 3-47-4 | m/z = 715.27 (C₅₁H₃₃N₅ = 715.84) | 3-48-4 | m/z = 714.28 (C₅₂H₃₄N₄ = 714.85) |
| 3-49-4 | m/z = 714.28 (C₅₂H₃₄N₄ = 714.85) | 3-50-4 | m/z = 715.27 (C₅₁H₃₃N₅ = 715.84) |
| 3-51-4 | m/z = 716.27 (C₅₀H₃₂N₆ = 716.83) | 3-52-4 | m/z = 613.23 (C₄₃H₂₇N₅ = 613.71) |
| 3-53-4 | m/z = 485.19 (C₃₅H₂₃N₃ = 485.58) | 3-54-4 | m/z = 535.20 (C₃₉H₂₅N₃ = 535.64) |
| 3-55-4 | m/z = 561.22 (C₄₁H₂₂N₃ = 561.67) | 3-56-4 | m/z = 640.24 (C₄₄H₂₈N₆ = 640.73) |
| 3-57-4 | m/z = 485.19 (C₃₅H₂₃N₃ = 485.58) | 3-58-4 | m/z = 535.20 (C₃₉H₂₅N₃ = 535.64) |
| 3-59-4 | m/z = 561.22 (C₄₁H₂₂N₃ = 561.67) | 3-60-4 | m/z = 640.24 (C₄₄H₂₈N₆ = 640.73) |
| 3-61-4 | m/z = 485.19 (C₃₅H₂₃N₃ = 485.58) | 3-62-4 | m/z = 561.22 (C₄₁H₂₂N₃ = 561.67) |
| 3-63-4 | m/z = 561.22 (C₄₁H₂₂N₃ = 561.67) | 3-64-4 | m/z = 637.25 (C₄₇H₃₁N₃ = 637.77) |
| 3-65-4 | m/z = 637.25 (C₄₇H₃₁N₃ = 637.77) | 3-66-4 | m/z = 637.25 (C₄₇H₃₁N₃ = 637.77) |
| 3-67-4 | m/z = 637.25 (C₄₇H₃₁N₃ = 637.77) | 3-68-4 | m/z = 640.24 (C₄₄H₂₈N₆ = 640.73) |
| 3-69-4 | m/z = 639.24 (C₄₅H₂₉N₅ = 639.75) | 3-70-4 | m/z = 639.24 (C₄₅H₂₉N₅ = 639.75) |
| 3-71-4 | m/z = 639.24 (C₄₅H₂₉N₅ = 639.75) | 3-72-4 | m/z = 638.25 (C₄₆H₃₀N₄ = 638.76) |
| 3-73-4 | m/z = 638.25 (C₄₆H₃₀N₄ = 638.76) | 3-74-4 | m/z = 639.24 (C₄₅H₂₉N₅ = 639.75) |
| 3-75-4 | m/z = 640.24 (C₄₄H₂₈N₆ = 640.73) | 3-76-4 | m/z = 716.27 (C₅₀H₃₂N₆ = 716.83) |
| 3-77-4 | m/z = 715.27 (C₅₁H₃₃N₅ = 715.84) | 3-78-4 | m/z = 715.27 (C₅₁H₃₃N₅ = 715.84) |
| 3-79-4 | m/z = 714.28 (C₅₂H₃₄N₄ = 714.85) | 3-80-4 | m/z = 714.28 (C₅₂H₃₄N₄ = 714.85) |
| 3-81-4 | m/z = 715.27 (C₅₁H₃₃N₅ = 715.84) | 3-82-4 | m/z = 716.27 (C₅₀H₃₂N₆ = 716.83) |
| 3-83-4 | m/z = 716.27 (C₅₀H₃₂N₆ = 716.83) | 3-84-4 | m/z = 715.27 (C₅₁H₃₃N₅ = 715.84) |
| 3-85-4 | m/z = 715.27 (C₅₁H₃₃N₅ = 715.84) | 3-86-4 | m/z = 714.28 (C₅₂H₃₄N₄ = 714.85) |
| 3-87-4 | m/z = 714.28 (C₅₂H₃₄N₄ = 714.85) | 3-88-4 | m/z = 715.27 (C₅₁H₃₃N₅ = 715.84) |
| 3-89-4 | m/z = 716.27 (C₅₀H₃₂N₆ = 716.83) | 3-90-4 | m/z = 613.23 (C₄₃H₂₇N₅ = 613.71) |
| 3-91-4 | m/z = 640.24 (C₄₄H₂₈N₆ = 640.73) | 3-92-4 | m/z = 639.24 (C₄₅H₂₉N₅ = 639.75) |
| 3-93-4 | m/z = 639.24 (C₄₅H₂₉N₅ = 639.75) | 3-94-4 | m/z = 639.24 (C₄₅H₂₉N₅ = 639.75) |
| 3-95-4 | m/z = 638.25 (C₄₆H₃₀N₄ = 638.76) | 3-96-4 | m/z = 638.25 (C₄₆H₃₀N₄ = 638.76) |
| 3-97-4 | m/z = 639.24 (C₄₅H₂₉N₅ = 639.75) | 3-98-4 | m/z = 640.24 (C₄₄H₂₈N₆ = 640.73) |
| 3-99-4 | m/z = 716.27 (C₅₀H₃₂N₆ = 716.83) | 3-100-4 | m/z = 715.27 (C₅₁H₃₃N₅ = 715.84) |
| 3-101-4 | m/z = 715.27 (C₅₁H₃₃N₅ = 715.84) | 3-102-4 | m/z = 714.28 (C₅₂H₃₄N₄ = 714.85) |
| 3-103-4 | m/z = 714.28 (C₅₂H₃₄N₄ = 714.85) | 3-104-4 | m/z = 715.27 (C₅₁H₃₃N₅ = 715.84) |
| 3-105-4 | m/z = 716.27 (C₅₀H₃₂N₆ = 716.83) | 3-106-4 | m/z = 716.27 (C₅₀H₃₂N₆ = 716.83) |
| 3-107-4 | m/z = 715.27 (C₅₁H₃₃N₅ = 715.84) | 3-108-4 | m/z = 715.27 (C₅₁H₃₃N₅ = 715.84) |
| 3-109-4 | m/z = 714.28 (C₅₂H₃₄N₄ = 714.85) | 3-110-4 | m/z = 714.28 (C₅₂H₃₄N₄ = 714.85) |
| 3-111-4 | m/z = 715.27 (C₅₁H₃₃N₅ = 715.84) | 3-112-4 | m/z = 716.27 (C₅₀H₃₂N₆ = 716.83) |
| 3-113-4 | m/z = 613.23 (C₄₃H₂₇N₅ = 613.71) | 3-114-4 | m/z = 485.19 (C₃₅H₂₃N₃ = 485.58) |
| 3-115-4 | m/z = 535.20 (C₃₉H₂₅N₃ = 535.64) | 3-116-4 | m/z = 561.22 (C₄₁H₂₂N₃ = 561.67) |
| 3-117-4 | m/z = 640.24 (C₄₄H₂₈N₆ = 640.73) | 3-118-4 | m/z = 485.19 (C₃₅H₂₃N₃ = 485.58) |
| 3-119-4 | m/z = 535.20 (C₃₉H₂₅N₃ = 535.64) | 3-120-4 | m/z = 561.22 (C₄₁H₂₂N₃ = 561.67) |
| 5-121-4 | m/z = 640.24 (C₄₄H₂₈N₆ = 640.73) | 3-122-4 | m/z = 485.19 (C₃₅H₂₃N₃ = 485.58) |
| 3-123-4 | m/z = 535.20 (C₃₉H₂₅N₃ = 535.64) | 3-124-4 | m/z = 561.22 (C₄₁H₂₂N₃ = 561.67) |
| 3-125-4 | m/z = 640.24 (C₄₄H₂₈N₆ = 640.73) | 3-126-4 | m/z = 535.20 (C₃₉H₂₅N₃ = 535.64) |
| 3-127-4 | m/z = 535.20 (C₃₉H₂₅N₃ = 535.64) | 4-1-4 | m/z = 485.19 (C₃₅H₂₃N₃ = 485.58) |
| 4-2-4 | m/z = 535.20 (C₃₉H₂₅N₃ = 535.64) | 4-3-4 | m/z = 561.22 (C₄₁H₂₂N₃ = 561.67) |
| 4-4-4 | m/z = 640.24 (C₄₄H₂₈N₆ = 640.73) | 4-5-4 | m/z = 485.19 (C₃₅H₂₃N₃ = 485.58) |
| 4-6-4 | m/z = 535.20 (C₃₉H₂₅N₃ = 535.64) | 4-7-4 | m/z = 561.22 (C₄₁H₂₂N₃ = 561.67) |
| 4-8-4 | m/z = 640.24 (C₄₄H₂₈N₆ = 640.73) | 4-9-4 | m/z = 485.19 (C₃₅H₂₃N₃ = 485.58) |
| 4-10-4 | m/z = 535.20 (C₃₉H₂₅N₃ = 535.64) | 4-11-4 | m/z = 561.22 (C₄₁H₂₂N₃ = 561.67) |
| 4-12-4 | m/z = 640.24 (C₄₄H₂₈N₆ = 640.73) | 4-13-4 | m/z = 485.19 (C₃₅H₂₃N₃ = 485.58) |
| 4-14-4 | m/z = 535.20 (C₃₉H₂₅N₃ = 535.64) | 4-15-4 | m/z = 561.22 (C₄₁H₂₂N₃ = 561.67) |
| 4-16-4 | m/z = 640.24 (C₄₄H₂₈N₆ = 640.73) | 4-17-4 | m/z = 485.19 (C₃₅H₂₃N₃ = 485.58) |
| 4-18-4 | m/z = 535.20 (C₃₉H₂₅N₃ = 535.64) | 4-19-4 | m/z = 561.22 (C₄₁H₂₂N₃ = 561.67) |
| 4-20-4 | m/z = 640.24 (C₄₄H₂₈N₆ = 640.73) | 4-21-4 | m/z = 485.19 (C₃₅H₂₃N₃ = 485.58) |
| 4-22-4 | m/z = 535.20 (C₃₉H₂₅N₃ = 535.64) | 4-23-4 | m/z = 561.22 (C₄₁H₂₂N₃ = 561.67) |
| 4-24-4 | m/z = 640.24 (C₄₄H₂₈N₆ = 640.73) | 4-25-4 | m/z = 485.19 (C₃₅H₂₃N₃ = 485.58) |
| 4-26-4 | m/z = 535.20 (C₃₉H₂₅N₃ = 535.64) | 4-27-4 | m/z = 561.22 (C₄₁H₂₂N₃ = 561.67) |
| 4-28-4 | m/z = 640.24 (C₄₄H₂₈N₆ = 640.73) | | |
| 5-1-4 | m/z = 653.26 (C₄₆H₃₁N₅ = 653.77) | 5-3-4 | m/z = 652.26 (C₄₇H₃₂N₄ = 652.78) |
| 5-2-4 | m/z = 728.29 (C₅₃H₃₆N₄ = 728.88) | 5-4-4 | m/z = 728.29 (C₅₃H₃₆N₄ = 728.88) |

### Manufacture and Evaluation of Organic Electronic Element

### □. Manufacture and Test of Green Organic Light Emitting Element (Phosphorescent Host)

### Example 4-1 Green Organic Light Emitting Element (Phosphorescent Host)

An organic electronic light emitting element was manufactured by an ordinary method using the compound obtained through synthesis as a host material for a light emitting layer. First, a film of N¹-(naphthalen-2-yl)-N⁴,N⁴-bis(4-(naphthalen-2-yl(phenyl)amino)phenyl)-N¹-phenylbenzene-1,4-diamine (hereinafter, abbreviated as “2-TNATA”) as a hole injection layer was vacuum-deposited with a thickness of 60 nm on an ITO layer (anode) formed on a galas substrate. Then, 4,4-bis[N-(1-naphthyl)-N-phenylamino]biphenyl (hereinafter, abbreviated as “-NPD”) as a hole transport compound was vacuum-deposited on the hole injection layer to form a hole transport layer with a thickness of 60 nm. Subsequently, a light emitting layer with a thickness of 30 nm was formed on the hole transport layer by doping an upper portion of the hole transport layer with the compound 1-1-4 of the present invention as a host and Ir(ppy)₃ [tris(2-phenylpyridine)-iridium] as a dopant at a weight ratio of 95:5. Then, (1.1′-bisphenyl)-4-olato)bis(2-methyl-8-quinolinolato)aluminum (hereinafter, abbreviated as “BAlq”) was vacuum-deposited with a thickness of 10 nm for a hole blocking layer, and tris(8-quinolinol)aluminum (hereinafter, abbreviated as “Alq3”) was formed with a thickness of 40 nm for an electron injection layer. Thereafter, LiF as halogenated alkali metal was deposited with a thickness of 0.2 nm, and subsequently Al was deposited with a thickness of 150 nm, thereby using this Al/LiF as a cathode. In this way, an organic electronic light emitting element was manufactured.

### Example 4-2 to Example 4-184 Green Organic Light Emitting Element (Phosphorescent Host)

An organic electronic light emitting element was manufactured by the same method as in Example 4-1 except that, instead of compound 1-1-4 of the present invention, one of compounds 1-2-4 to 1-28-4, 2-1-4 to 2-128-4, and 4-1-4 to 4-28-4 of the present invention listed on table 5 below was used as a phosphorescent host material for a light emitting layer.

### Comparative Example 4-1

An organic electronic light emitting element was manufactured by the same method as in Example 4-1 except that, instead of compound 1-1-4 of the present invention, comparative compound A [4,4′-N,N′-dicarbazole-biphenyl (CBP)] described in <Example 1> was used as a phosphorescent host material for a light emitting layer.

### Comparative Example 4-2

An organic electronic light emitting element was manufactured by the same method as in Example 4-1 except that, instead of compound 1-1-4 of the present invention, comparative compound B described in <Example 1> was used as a phosphorescent host material for a light emitting layer.

### Comparative Example 4-3

An organic electronic light emitting element was manufactured by the same method as in Example 4-1 except that, instead of compound 1-1-4 of the present invention, comparative compound C described in <Example 1> was used as a phosphorescent host material for a light emitting layer.

### Comparative Example 4-4

An organic electronic light emitting element was manufactured by the same method as in Example 4-1 except that, instead of compound 1-1-4 of the present invention, comparative compound D describe in <Example 1> was used as a phosphorescent host material for a light emitting layer.

A forward bias DC voltage was applied to the organic electronic light emitting elements manufactured in Examples 4-1 to 4-184 and Comparative Examples 4-1 to 4-4 to measure electro-luminescence (EL) characteristics thereof by PR-650 (Photoresearch), and the T95 lifetime was measured by lifetime measuring equipments (Mcscience) at reference brightness of 5000 cd/m². Table 4-5 below shows the manufacture of elements and evaluation results thereof.

TABLE 4-5

| | | | Current | Brightness | | Lifetime | CIE |
|---|---|---|---|---|---|---|---|
| | Compound | Voltage | Density | (cd/m2) | Efficiency | T(95) | (x, y) |
| Comparative | Compound | 5.8 | 23.1 | 5000.0 | 21.6 | 65.8 | (0.31, |
| Example(4-1) | (A) | | | | | | 0.60) |
| Comparative | Compound | 5.2 | 16.9 | 5000.0 | 29.5 | 98.7 | (0.31, |
| Example(4-2) | (B) | | | | | | 0.61) |
| Comparative | Compound | 5.4 | 18.7 | 5000.0 | 26.7 | 91.1 | (0.31, |
| Example(4-3) | (C) | | | | | | 0.60) |
| Comparative | Compound | 5.5 | 17.3 | 5000.0 | 28.9 | 94.3 | (0.33, |
| Example(4-4) | (D) | | | | | | 0.61) |
| Example(4-1) | Compound | 5.0 | 14.5 | 5000.0 | 34.5 | 119.6 | (0.30, |
| | (1-1-4) | | | | | | 0.60) |
| Example(4-2) | Compound | 4.8 | 16.2 | 5000.0 | 30.8 | 97.9 | (0.31, |
| | (1-2-4) | | | | | | 0.61) |
| Example(4-3) | Compound | 5.0 | 15.5 | 5000.0 | 32.2 | 120.5 | (0.31, |
| | (1-3-4) | | | | | | 0.60) |
| Example(4-4) | Compound | 5.0 | 16.2 | 5000.0 | 30.9 | 94.1 | (0.33, |
| | (1-4-4) | | | | | | 0.61) |
| Example(4-5) | Compound | 4.8 | 14.5 | 5000.0 | 34.5 | 94.2 | (0.32, |
| | (1-5-4) | | | | | | 0.61) |
| Example(4-6) | Compound | 4.9 | 15.5 | 5000.0 | 32.3 | 99.5 | (0.33, |
| | (1-6-4) | | | | | | 0.60) |
| Example(4-7) | Compound | 4.8 | 16.6 | 5000.0 | 30.2 | 116.4 | (0.32, |
| | (1-7-4) | | | | | | 0.61) |
| Example(4-8) | Compound | 4.9 | 15.7 | 5000.0 | 31.8 | 92.1 | (0.31, |
| | (1-8-4) | | | | | | 0.60) |
| Example4-(9) | Compound | 4.9 | 14.7 | 5000.0 | 34.0 | 99.4 | (0.31, |
| | (1-9-4) | | | | | | 0.61) |
| Example(4-10) | Compound | 4.9 | 15.1 | 5000.0 | 33.1 | 121.9 | (0.31, |
| | (1-10-4) | | | | | | 0.60) |
| Example(4-11) | Compound | 5.0 | 16.4 | 5000.0 | 30.5 | 113.6 | (0.33, |
| | (1-11-4) | | | | | | 0.61) |
| Example(4-12) | Compound | 4.9 | 16.2 | 5000.0 | 31.0 | 102.9 | (0.30, |
| | (1-12-4) | | | | | | 0.60) |
| Example(4-13) | Compound | 5.0 | 14.8 | 5000.0 | 33.7 | 109.7 | (0.31, |
| | (1-13-4) | | | | | | 0.61) |
| Example(4-14) | Compound | 4.9 | 15.2 | 5000.0 | 32.9 | 126.5 | (0.31, |
| | (1-14-4) | | | | | | 0.60) |
| Example(4-15) | Compound | 5.0 | 15.2 | 5000.0 | 32.9 | 95.8 | (0.33, |
| | (1-15-4) | | | | | | 0.61) |
| Example(4-16) | Compound | 4.9 | 14.7 | 5000.0 | 34.1 | 96.9 | (0.32, |
| | (1-16-4) | | | | | | 0.61) |
| Example(4-17) | Compound | 5.0 | 16.6 | 5000.0 | 30.1 | 105.0 | (0.33, |
| | (1-17-4) | | | | | | 0.60) |
| Example(4-18) | Compound | 4.9 | 14.3 | 5000.0 | 35.0 | 122.7 | (0.32, |
| | (1-18-4) | | | | | | 0.61) |
| Example(4-19) | Compound | 4.8 | 14.4 | 5000.0 | 34.8 | 124.2 | (0.31, |
| | (1-19-4) | | | | | | 0.60) |
| Example(4-20) | Compound | 4.9 | 14.8 | 5000.0 | 33.8 | 95.4 | (0.31, |
| | (1-20-4) | | | | | | 0.61) |
| Example(4-21) | Compound | 4.9 | 15.9 | 5000.0 | 31.5 | 90.7 | (0.31, |
| | (1-21-4) | | | | | | 0.60) |
| Example(4-22) | Compound | 5.0 | 15.8 | 5000.0 | 31.7 | 106.2 | (0.33, |
| | (1-22-4) | | | | | | 0.61) |
| Example(4-23) | Compound | 4.9 | 16.3 | 5000.0 | 30.6 | 116.5 | (0.30, |
| | (1-23-4) | | | | | | 0.60) |
| Example(4-24) | Compound | 5.0 | 14.7 | 5000.0 | 34.1 | 113.9 | (0.31, |
| | (1-24-4) | | | | | | 0.61) |
| Example(4-25) | Compound | 5.0 | 14.4 | 5000.0 | 34.6 | 128.9 | (0.31, |
| | (1-25-4) | | | | | | 0.60) |
| Example(4-26) | Compound | 4.9 | 14.9 | 5000.0 | 33.6 | 99.7 | (0.33, |
| | (1-26-4) | | | | | | 0.61) |
| Example(4-27) | Compound | 4.8 | 15.9 | 5000.0 | 31.5 | 122.4 | (0.32, |
| | (1-27-4) | | | | | | 0.61) |
| Example(4-28) | Compound | 4.9 | 14.5 | 5000.0 | 34.5 | 107.6 | (0.33, |
| | (1-28-4) | | | | | | 0.60) |
| Example(4-29) | Compound | 4.5 | 14.1 | 5000.0 | 35.5 | 132.6 | (0.31, |
| | (2-1-4) | | | | | | 0.61) |
| Example(4-30) | Compound | 4.7 | 13.7 | 5000.0 | 36.4 | 137.5 | (0.31, |
| | (2-2-4) | | | | | | 0.60) |
| Example(4-31) | Compound | 4.7 | 13.4 | 5000.0 | 37.2 | 130.0 | (0.33, |
| | (2-3-4) | | | | | | 0.61) |
| Example(4-32) | Compound | 4.6 | 14.0 | 5000.0 | 35.6 | 133.4 | (0.32, |
| | (2-4-4) | | | | | | 0.61) |
| Example(4-33) | Compound | 4.6 | 12.9 | 5000.0 | 38.7 | 138.7 | (0.33, |
| | (2-5-4) | | | | | | 0.60) |
| Example(4-34) | Compound | 4.6 | 13.8 | 5000.0 | 36.4 | 134.1 | (0.32, |
| | (2-6-4) | | | | | | 0.61) |
| Example(4-35) | Compound | 4.7 | 12.8 | 5000.0 | 39.2 | 139.7 | (0.31, |
| | (2-7-4) | | | | | | 0.60) |
| Example(4-36) | Compound | 4.7 | 13.5 | 5000.0 | 37.2 | 137.6 | (0.31, |
| | (2-8-4) | | | | | | 0.61) |
| Example(4-37) | Compound | 4.6 | 12.8 | 5000.0 | 39.1 | 138.2 | (0.31, |
| | (2-9-4) | | | | | | 0.60) |
| Example(4-38) | Compound | 4.6 | 13.1 | 5000.0 | 38.3 | 137.3 | (0.33, |
| | (2-10-4) | | | | | | 0.61) |
| Example(4-39) | Compound | 4.7 | 12.6 | 5000.0 | 39.6 | 134.2 | (0.30, |
| | (2-11-4) | | | | | | 0.60) |
| Example(4-40) | Compound | 4.7 | 12.7 | 5000.0 | 39.4 | 131.8 | (0.31, |
| | (2-12-4) | | | | | | 0.61) |
| Example(4-41) | Compound | 4.5 | 13.2 | 5000.0 | 38.0 | 139.2 | (0.31, |
| | (2-13-4) | | | | | | 0.60) |
| Example(4-42) | Compound | 4.7 | 13.2 | 5000.0 | 37.9 | 130.7 | (0.33, |
| | (2-14-4) | | | | | | 0.61) |
| Example(4-43) | Compound | 4.7 | 14.2 | 5000.0 | 35.2 | 135.8 | (0.32, |
| | (2-15-4) | | | | | | 0.61) |
| Example(4-44) | Compound | 4.5 | 13.9 | 5000.0 | 36.0 | 131.2 | (0.33, |
| | (2-16-4) | | | | | | 0.60) |
| Example(4-45) | Compound | 4.6 | 13.1 | 5000.0 | 38.2 | 133.9 | (0.32, |
| | (2-17-4) | | | | | | 0.61) |
| Example(4-46) | Compound | 4.7 | 13.3 | 5000.0 | 37.5 | 132.6 | (0.31, |
| | (2-18-4) | | | | | | 0.60) |
| Example(4-47) | Compound | 4.6 | 13.2 | 5000.0 | 38.0 | 138.9 | (0.31, |
| | (2-19-4) | | | | | | 0.61) |
| Example(4-48) | Compound | 4.6 | 14.2 | 5000.0 | 35.1 | 133.0 | (0.31, |
| | (2-20-4) | | | | | | 0.60) |
| Example(4-49) | Compound | 4.6 | 13.4 | 5000.0 | 37.2 | 138.3 | (0.33, |
| | (2-21-4) | | | | | | 0.61) |
| Example(4-50) | Compound | 4.6 | 12.7 | 5000.0 | 39.3 | 130.3 | (0.30, |
| | (2-22-4) | | | | | | 0.60) |
| Example(4-51) | Compound | 4.6 | 13.0 | 5000.0 | 38.6 | 136.6 | (0.31, |
| | (2-23-4) | | | | | | 0.61) |
| Example(4-52) | Compound | 4.7 | 12.9 | 5000.0 | 38.7 | 138.8 | (0.31, |
| | (2-24-4) | | | | | | 0.60) |
| Example(4-53) | Compound | 4.6 | 13.2 | 5000.0 | 37.9 | 131.8 | (0.33, |
| | (2-25-4) | | | | | | 0.61) |
| Example(4-54) | Compound | 4.6 | 13.9 | 5000.0 | 36.1 | 138.5 | (0.32, |
| | (2-26-4) | | | | | | 0.61) |
| Example(4-55) | Compound | 4.7 | 12.9 | 5000.0 | 38.8 | 131.1 | (0.33, |
| | (2-27-4) | | | | | | 0.60) |
| Example(4-56) | Compound | 4.6 | 12.6 | 5000.0 | 39.8 | 130.5 | (0.32, |
| | (2-28-4) | | | | | | 0.61) |
| Example(4-57) | Compound | 4.6 | 14.0 | 5000.0 | 35.6 | 132.8 | (0.31, |
| | (2-29-4) | | | | | | 0.60) |
| Example(4-58) | Compound | 4.5 | 12.7 | 5000.0 | 39.4 | 131.5 | (0.33, |
| | (2-30-4) | | | | | | 0.61) |
| Example(4-59) | Compound | 4.5 | 13.9 | 5000.0 | 36.1 | 133.1 | (0.30, |
| | (2-31-4) | | | | | | 0.60) |
| Example(4-60) | Compound | 4.7 | 13.8 | 5000.0 | 36.2 | 137.6 | (0.31, |
| | (2-32-4) | | | | | | 0.61) |
| Example(4-61) | Compound | 4.5 | 12.9 | 5000.0 | 38.8 | 130.5 | (0.31, |
| | (2-33-4) | | | | | | 0.60) |
| Example(4-62) | Compound | 4.6 | 13.7 | 5000.0 | 36.4 | 137.1 | (0.33, |
| | (2-34-4) | | | | | | 0.61) |
| Example(4-63) | Compound | 4.5 | 13.1 | 5000.0 | 38.1 | 130.7 | (0.32, |
| | (2-35-4) | | | | | | 0.61) |
| Example(4-64) | Compound | 4.6 | 13.1 | 5000.0 | 38.2 | 135.4 | (0.33, |
| | (2-36-4) | | | | | | 0.60) |
| Example(4-65) | Compound | 4.6 | 12.7 | 5000.0 | 39.5 | 135.9 | (0.32, |
| | (2-37-4) | | | | | | 0.61) |
| Example(4-66) | Compound | 4.5 | 13.0 | 5000.0 | 38.4 | 131.8 | (0.31, |
| | (2-38-4) | | | | | | 0.60) |
| Example(4-67) | Compound | 4.7 | 13.1 | 5000.0 | 38.2 | 136.5 | (0.31, |
| | (2-39-4) | | | | | | 0.61) |
| Example(4-68) | Compound | 4.6 | 13.0 | 5000.0 | 38.3 | 138.4 | (0.31, |
| | (2-40-4) | | | | | | 0.60) |
| Example(4-69) | Compound | 4.6 | 13.2 | 5000.0 | 37.9 | 130.0 | (0.33, |
| | (2-41-4) | | | | | | 0.61) |
| Example(4-70) | Compound | 4.6 | 13.1 | 5000.0 | 38.1 | 137.5 | (0.30, |
| | (2-42-4) | | | | | | 0.60) |
| Example(4-71) | Compound | 4.7 | 13.5 | 5000.0 | 37.1 | 137.4 | (0.31, |
| | (2-43-4) | | | | | | 0.61) |
| Example(4-72) | Compound | 4.6 | 14.3 | 5000.0 | 35.1 | 136.3 | (0.31, |
| | (2-44-4) | | | | | | 0.60) |
| Example(4-73) | Compound | 4.6 | 13.3 | 5000.0 | 37.7 | 135.0 | (0.33, |
| | (2-45-4) | | | | | | 0.61) |
| Example(4-74) | Compound | 4.6 | 12.6 | 5000.0 | 39.6 | 135.8 | (0.32, |
| | (2-46-4) | | | | | | 0.61) |
| Example(4-75) | Compound | 4.5 | 13.9 | 5000.0 | 36.1 | 137.2 | (0.33, |
| | (2-47-4) | | | | | | 0.61) |
| Example(4-76) | Compound | 4.5 | 12.7 | 5000.0 | 39.3 | 134.7 | (0.30, |
| | (2-48-4) | | | | | | 0.60) |
| Example(4-77) | Compound | 4.6 | 13.6 | 5000.0 | 36.7 | 135.6 | (0.31, |
| | (2-49-4) | | | | | | 0.61) |
| Example(4-78) | Compound | 4.5 | 13.6 | 5000.0 | 36.6 | 132.2 | (0.31, |
| | (2-50-4) | | | | | | 0.60) |
| Example(4-79) | Compound | 4.6 | 13.6 | 5000.0 | 36.8 | 137.9 | (0.31, |
| | (2-51-4) | | | | | | 0.61) |
| Example(4-80) | Compound | 4.6 | 13.9 | 5000.0 | 35.9 | 134.9 | (0.31, |
| | (2-52-4) | | | | | | 0.60) |
| Example(4-81) | Compound | 4.6 | 13.6 | 5000.0 | 36.8 | 135.9 | (0.33, |
| | (2-53-4) | | | | | | 0.61) |
| Example(4-82) | Compound | 4.7 | 12.7 | 5000.0 | 39.3 | 136.1 | (0.32, |
| | (2-54-4) | | | | | | 0.61) |
| Example(4-83) | Compound | 4.6 | 13.8 | 5000.0 | 36.2 | 131.9 | (0.33, |
| | (2-55-4) | | | | | | 0.60) |
| Example(4-84) | Compound | 4.7 | 14.1 | 5000.0 | 35.4 | 133.7 | (0.32, |
| | (2-56-4) | | | | | | 0.61) |
| Example(4-85) | Compound | 4.6 | 13.0 | 5000.0 | 38.6 | 138.4 | (0.31, |
| | (2-57-4) | | | | | | 0.60) |
| Example(4-86) | Compound | 4.6 | 12.6 | 5000.0 | 39.7 | 132.3 | (0.31, |
| | (2-58-4) | | | | | | 0.61) |
| Example(4-87) | Compound | 4.7 | 13.7 | 5000.0 | 36.4 | 137.6 | (0.31, |
| | (2-59-4) | | | | | | 0.60) |
| Example(4-88) | Compound | 4.6 | 13.7 | 5000.0 | 36.4 | 136.7 | (0.33, |
| | (2-60-4) | | | | | | 0.61) |
| Example(4-89) | Compound | 4.7 | 12.6 | 5000.0 | 39.5 | 139.3 | (0.30, |
| | (2-61-4) | | | | | | 0.60) |
| Example(4-90) | Compound | 4.6 | 13.5 | 5000.0 | 37.2 | 131.7 | (0.31, |
| | (2-62-4) | | | | | | 0.61) |
| Example(4-91) | Compound | 4.6 | 13.8 | 5000.0 | 36.1 | 132.2 | (0.31, |
| | (2-63-4) | | | | | | 0.60) |
| Example(4-92) | Compound | 4.6 | 13.4 | 5000.0 | 37.3 | 133.4 | (0.33, |
| | (2-64-4) | | | | | | 0.61) |
| Example(4-93) | Compound | 4.5 | 12.8 | 5000.0 | 39.0 | 135.3 | (0.32, |
| | (2-65-4) | | | | | | 0.61) |
| Example(4-94) | Compound | 4.6 | 12.6 | 5000.0 | 39.6 | 135.9 | (0.33, |
| | (2-66-4) | | | | | | 0.60) |
| Example(4-95) | Compound | 4.7 | 14.3 | 5000.0 | 35.0 | 138.5 | (0.32, |
| | (2-67-4) | | | | | | 0.61) |
| Example(4-96) | Compound | 4.5 | 13.1 | 5000.0 | 38.2 | 132.4 | (0.31, |
| | (2-68-4) | | | | | | 0.60) |
| Example(4-97) | Compound | 4.6 | 13.2 | 5000.0 | 37.9 | 135.6 | (0.31, |
| | (2-69-4) | | | | | | 0.61) |
| Example(4-98) | Compound | 4.5 | 13.5 | 5000.0 | 37.1 | 138.9 | (0.31, |
| | (2-70-4) | | | | | | 0.60) |
| Example(4-99) | Compound | 4.5 | 12.7 | 5000.0 | 39.4 | 139.2 | (0.33, |
| | (2-71-4) | | | | | | 0.61) |
| Example(4-100) | Compound | 4.6 | 12.6 | 5000.0 | 39.6 | 139.9 | (0.30, |
| | (2-72-4) | | | | | | 0.60) |
| Example(4-101) | Compound | 4.7 | 12.6 | 5000.0 | 39.6 | 133.6 | (0.31, |
| | (2-73-4) | | | | | | 0.61) |
| Example(4-102) | Compound | 4.6 | 12.8 | 5000.0 | 39.1 | 131.8 | (0.31, |
| | (2-74-4) | | | | | | 0.60) |
| Example(4-103) | Compound | 4.5 | 14.1 | 5000.0 | 35.5 | 139.7 | (0.33, |
| | (2-75-4) | | | | | | 0.61) |
| Example(4-104) | Compound | 4.5 | 13.2 | 5000.0 | 38.0 | 131.5 | (0.32, |
| | (2-76-4) | | | | | | 0.61) |
| Example(4-105) | Compound | 4.6 | 13.9 | 5000.0 | 35.9 | 134.1 | (0.33, |
| | (2-77-4) | | | | | | 0.60) |
| Example(4-106) | Compound | 4.5 | 12.6 | 5000.0 | 39.7 | 134.4 | (0.32, |
| | (2-78-4) | | | | | | 0.61) |
| Example(4-107) | Compound | 4.7 | 12.9 | 5000.0 | 38.8 | 130.5 | (0.31, |
| | (2-79-4) | | | | | | 0.60) |
| Example(4-108) | Compound | 4.5 | 12.9 | 5000.0 | 38.7 | 131.1 | (0.33, |
| | (2-80-4) | | | | | | 0.61) |
| Example(4-109) | Compound | 4.5 | 13.0 | 5000.0 | 38.4 | 136.5 | (0.30, |
| | (2-81-4) | | | | | | 0.60) |
| Example(4-110) | Compound | 4.5 | 12.9 | 5000.0 | 38.8 | 139.9 | (0.31, |
| | (2-82-4) | | | | | | 0.61) |
| Example(4-111) | Compound | 4.5 | 14.0 | 5000.0 | 35.6 | 135.2 | (0.31, |
| | (2-83-4) | | | | | | 0.60) |
| Example(4-112) | Compound | 4.6 | 12.8 | 5000.0 | 39.2 | 136.9 | (0.33, |
| | (2-84-4) | | | | | | 0.61) |
| Example(4-113) | Compound | 4.6 | 13.0 | 5000.0 | 38.4 | 135.5 | (0.32, |
| | (2-85-4) | | | | | | 0.61) |
| Example(4-114) | Compound | 4.7 | 12.9 | 5000.0 | 38.8 | 133.2 | (0.33, |
| | (2-86-4) | | | | | | 0.60) |
| Example(4-115) | Compound | 4.6 | 13.5 | 5000.0 | 36.9 | 131.5 | (0.32, |
| | (2-87-4) | | | | | | 0.61) |
| Example(4-116) | Compound | 4.7 | 12.6 | 5000.0 | 39.8 | 134.8 | (0.31, |
| | (2-88-4) | | | | | | 0.60) |
| Example(4-117) | Compound | 4.6 | 12.5 | 5000.0 | 40.0 | 137.5 | (0.31, |
| | (2-89-4) | | | | | | 0.61) |
| Example(4-118) | Compound | 4.6 | 13.7 | 5000.0 | 36.5 | 132.2 | (0.31, |
| | (2-90-4) | | | | | | 0.60) |
| Example(4-119) | Compound | 4.6 | 13.7 | 5000.0 | 36.4 | 135.6 | (0.33, |
| | (2-91-4) | | | | | | 0.61) |
| Example(4-120) | Compound | 4.6 | 14.3 | 5000.0 | 35.1 | 139.3 | (0.30, |
| | (2-92-4) | | | | | | 0.60) |
| Example(4-121) | Compound | 4.7 | 14.2 | 5000.0 | 35.2 | 137.4 | (0.31, |
| | (2-93-4) | | | | | | 0.61) |
| Example(4-122) | Compound | 4.5 | 14.3 | 5000.0 | 35.0 | 132.3 | (0.31, |
| | (2-94-4) | | | | | | 0.60) |
| Example(4-123) | Compound | 4.6 | 12.5 | 5000.0 | 39.9 | 136.8 | (0.33, |
| | (2-95-4) | | | | | | 0.61) |
| Example(4-124) | Compound | 4.7 | 12.9 | 5000.0 | 38.8 | 139.2 | (0.32, |
| | (2-96-4) | | | | | | 0.61) |
| Example(4-125) | Compound | 4.6 | 14.0 | 5000.0 | 35.7 | 131.4 | (0.33, |
| | (2-97-4) | | | | | | 0.61) |
| Example(4-126) | Compound | 4.7 | 13.3 | 5000.0 | 37.6 | 137.3 | (0.30, |
| | (2-98-4) | | | | | | 0.60) |
| Example(4-127) | Compound | 4.7 | 14.3 | 5000.0 | 35.0 | 135.4 | (0.32, |
| | (2-99-4) | | | | | | 0.61) |
| Example(4-128) | Compound | 4.7 | 14.2 | 5000.0 | 35.3 | 137.1 | (0.31, |
| | (2-100-4) | | | | | | 0.60) |
| Example(4-129) | Compound | 4.5 | 13.7 | 5000.0 | 36.5 | 133.6 | (0.30, |
| | (2-101-4) | | | | | | 0.60) |
| Example(4-130) | Compound | 4.7 | 13.4 | 5000.0 | 37.2 | 132.3 | (0.31, |
| | (2-102-4) | | | | | | 0.61) |
| Example(4-131) | Compound | 4.6 | 13.1 | 5000.0 | 38.2 | 132.8 | (0.31, |
| | (2-103-4) | | | | | | 0.60) |
| Example(4-132) | Compound | 4.6 | 12.5 | 5000.0 | 40.0 | 139.7 | (0.33, |
| | (2-104-4) | | | | | | 0.61) |
| Example(4-133) | Compound | 4.7 | 12.8 | 5000.0 | 39.1 | 138.6 | (0.32, |
| | (2-105-4) | | | | | | 0.61) |
| Example(4-134) | Compound | 4.6 | 12.5 | 5000.0 | 39.9 | 138.4 | (0.33, |
| | (2-106-4) | | | | | | 0.60) |
| Example(4-135) | Compound | 4.6 | 12.7 | 5000.0 | 39.4 | 130.2 | (0.32, |
| | (2-107-4) | | | | | | 0.61) |
| Example(4-136) | Compound | 4.7 | 14.2 | 5000.0 | 35.3 | 138.6 | (0.31, |
| | (2-108-4) | | | | | | 0.60) |
| Example(4-137) | Compound | 4.5 | 13.0 | 5000.0 | 38.4 | 137.2 | (0.31, |
| | (2-109-4) | | | | | | 0.61) |
| Example(4-138) | Compound | 4.5 | 13.4 | 5000.0 | 37.3 | 132.7 | (0.31, |
| | (2-110-4) | | | | | | 0.60) |
| Example(4-139) | Compound | 4.6 | 14.2 | 5000.0 | 35.3 | 138.8 | (0.33, |
| | (2-111-4) | | | | | | 0.61) |
| Example(4-140) | Compound | 4.5 | 14.0 | 5000.0 | 35.6 | 139.8 | (0.30, |
| | (2-112-4) | | | | | | 0.60) |
| Example(4-141) | Compound | 4.6 | 12.8 | 5000.0 | 38.9 | 135.4 | (0.31, |
| | (2-113-4) | | | | | | 0.61) |
| Example(4-142) | Compound | 4.6 | 13.6 | 5000.0 | 36.7 | 133.2 | (0.31, |
| | (2-114-4) | | | | | | 0.60) |
| Example(4-143) | Compound | 4.5 | 14.3 | 5000.0 | 35.1 | 132.6 | (0.33, |
| | (2-115-4) | | | | | | 0.61) |
| Example(4-144) | Compound | 4.7 | 13.3 | 5000.0 | 37.6 | 133.3 | (0.32, |
| | (2-116-4) | | | | | | 0.61) |
| Example(4-145) | Compound | 4.6 | 13.2 | 5000.0 | 37.9 | 131.3 | (0.33, |
| | (2-117-4) | | | | | | 0.60) |
| Example(4-146) | Compound | 4.6 | 13.0 | 5000.0 | 38.6 | 135.1 | (0.32, |
| | (2-118-4) | | | | | | 0.61) |
| Example(4-147) | Compound | 4.6 | 13.8 | 5000.0 | 36.2 | 134.3 | (0.31, |
| | (2-119-4) | | | | | | 0.60) |
| Example(4-148) | Compound | 4.7 | 13.7 | 5000.0 | 36.4 | 137.4 | (0.31, |
| | (2-120-4) | | | | | | 0.61) |
| Example(4-149) | Compound | 4.6 | 12.8 | 5000.0 | 39.1 | 131.9 | (0.31, |
| | (2-121-4) | | | | | | 0.60) |
| Example(4-150) | Compound | 4.5 | 13.1 | 5000.0 | 38.2 | 139.8 | (0.33, |
| | (2-122-4) | | | | | | 0.61) |
| Example(4-151) | Compound | 4.6 | 13.2 | 5000.0 | 37.8 | 135.5 | (0.30, |
| | (2-123-4) | | | | | | 0.60) |
| Example(4-152) | Compound | 4.6 | 14.1 | 5000.0 | 35.5 | 132.0 | (0.31, |
| | (2-124-4) | | | | | | 0.61) |
| Example(4-153) | Compound | 4.5 | 13.3 | 5000.0 | 37.6 | 139.9 | (0.31, |
| | (2-125-4) | | | | | | 0.60) |
| Example(4-154) | Compound | 4.5 | 13.4 | 5000.0 | 37.2 | 134.4 | (0.33, |
| | (2-126-4) | | | | | | 0.61) |
| Example(4-155) | Compound | 4.5 | 13.7 | 5000.0 | 36.6 | 134.6 | (0.32, |
| | (2-127-4) | | | | | | 0.61) |
| Example(4-156) | Compound | 4.6 | 13.5 | 5000.0 | 37.0 | 137.3 | (0.33, |
| | (2-128-4) | | | | | | 0.60) |
| Example(4-157) | Compound | 4.9 | 16.5 | 5000.0 | 30.3 | 108.7 | (0.31, |
| | (4-1-4) | | | | | | 0.61) |
| Example(4-158) | Compound | 4.9 | 14.5 | 5000.0 | 34.4 | 97.2 | (0.31, |
| | (4-2-4) | | | | | | 0.60) |
| Example(4-159) | Compound | 5.0 | 14.6 | 5000.0 | 34.3 | 121.2 | (0.33, |
| | (4-3-4) | | | | | | 0.61) |
| Example(4-160) | Compound | 4.9 | 15.2 | 5000.0 | 32.9 | 104.7 | (0.32, |
| | (4-4-4) | | | | | | 0.61) |
| Example(4-161) | Compound | 4.8 | 16.6 | 5000.0 | 30.0 | 112.2 | (0.33, |
| | (4-5-4) | | | | | | 0.60) |
| Example(4-162) | Compound | 4.8 | 16.0 | 5000.0 | 31.3 | 116.8 | (0.32, |
| | (4-6-4) | | | | | | 0.61) |
| Example(4-163) | Compound | 4.9 | 14.9 | 5000.0 | 33.6 | 99.7 | (0.31, |
| | (4-7-4) | | | | | | 0.60) |
| Example(4-164) | Compound | 4.8 | 16.0 | 5000.0 | 31.3 | 100.7 | (0.31, |
| | (4-8-4) | | | | | | 0.61) |
| Example(4-165) | Compound | 4.8 | 16.3 | 5000.0 | 30.6 | 127.6 | (0.31, |
| | (4-9-4) | | | | | | 0.60) |
| Example(4-166) | Compound | 4.9 | 15.6 | 5000.0 | 32.1 | 115.8 | (0.33, |
| | (4-10-4) | | | | | | 0.61) |
| Example(4-167) | Compound | 4.9 | 14.5 | 5000.0 | 34.4 | 121.6 | (0.30, |
| | (4-11-4) | | | | | | 0.60) |
| Example(4-168) | Compound | 4.9 | 16.1 | 5000.0 | 31.1 | 123.6 | (0.31, |
| | (4-12-4) | | | | | | 0.61) |
| Example(4-169) | Compound | 5.0 | 15.6 | 5000.0 | 32.1 | 96.2 | (0.31, |
| | (4-13-4) | | | | | | 0.60) |
| Example(4-170) | Compound | 5.0 | 15.7 | 5000.0 | 31.8 | 92.7 | (0.33, |
| | (4-14-4) | | | | | | 0.61) |
| Example(4-171) | Compound | 4.9 | 16.6 | 5000.0 | 30.1 | 102.8 | (0.32, |
| | (4-15-4) | | | | | | 0.61) |
| Example(4-172) | Compound | 4.8 | 16.5 | 5000.0 | 30.2 | 123.5 | (0.33, |
| | (4-16-4) | | | | | | 0.60) |
| Example(4-173) | Compound | 4.8 | 14.9 | 5000.0 | 33.6 | 95.3 | (0.32, |
| | (4-17-4) | | | | | | 0.61) |
| Example(4-174) | Compound | 4.9 | 16.1 | 5000.0 | 31.1 | 91.3 | (0.31, |
| | (4-18-4) | | | | | | 0.60) |
| Example(4-175) | Compound | 4.9 | 15.2 | 5000.0 | 32.8 | 107.1 | (0.31, |
| | (4-19-4) | | | | | | 0.61) |
| Example(4-176) | Compound | 5.0 | 15.6 | 5000.0 | 32.1 | 94.9 | (0.31, |
| | (4-20-4) | | | | | | 0.60) |
| Example(4-177) | Compound | 4.9 | 14.3 | 5000.0 | 34.9 | 122.0 | (0.33, |
| | (4-21-4) | | | | | | 0.61) |
| Example(4-178) | Compound | 5.0 | 15.5 | 5000.0 | 32.3 | 127.0 | (0.30, |
| | (4-22-4) | | | | | | 0.60) |
| Example(4-179) | Compound | 4.9 | 16.4 | 5000.0 | 30.5 | 98.0 | (0.31, |
| | (4-23-4) | | | | | | 0.61) |
| Example(4-180) | Compound | 4.8 | 15.2 | 5000.0 | 32.8 | 97.7 | (0.31, |
| | (4-24-4) | | | | | | 0.60) |
| Example(4-181) | Compound | 4.9 | 14.6 | 5000.0 | 34.3 | 100.6 | (0.33, |
| | (4-25-4) | | | | | | 0.61) |
| Example(4-182) | Compound | 5.0 | 16.1 | 5000.0 | 31.1 | 99.4 | (0.32, |
| | (4-26-4) | | | | | | 0.61) |
| Example(4-183) | Compound | 4.8 | 14.4 | 5000.0 | 34.7 | 100.4 | (0.33, |
| | (4-27-4) | | | | | | 0.60) |
| Example(4-184) | Compound | 4.8 | 16.2 | 5000.0 | 30.8 | 100.8 | (0.32, |
| | (4-28-4) | | | | | | 0.61) |

### H. Manufacture and Test of Red Organic Light Emitting Element (Phosphorescent Host)

### Example 4-185 Red Organic Light Emitting Element (Phosphorescent Host)

An organic electronic light emitting element was manufactured by an ordinary method using the compound obtained through synthesis as a light emitting host material for a light emitting layer. First, a film of N¹-(naphthalen-2-yl)-N⁴,N⁴-bis (4-(naphthalen-2-yl(phenyl)amino)phenyl)-N¹-phenylbenzene-1,4-diamine (hereinafter, abbreviated as “2-TNATA”) as a hole transport compound was vacuum-deposited on an ITO layer (anode) formed on a galas substrate to form a hole injection layer with a thickness 60 nm, and then, 4,4-bis[N-(1-naphthyl)-N-phenylamino]biphenyl (hereinafter, abbreviated as “-NPD”) as a hole transport compound was vacuum-deposited on the hole injection layer to form a hole transport layer with a thickness of 60 nm. Then, a light emitting layer with a thickness of 30 nm was deposited on the hole transport layer by doping an upper portion of the hole transport layer with compound 2-41-4 of the present invention as a host material and (piq)₂Ir(acac) [bis-(1-phenylisoquinolyl)iridium(III)acetylacetonate] as a dopant material at a weight ratio of 95:5. Then, (1.1′-bisphenyl)-4-olato)bis(2-methyl-8-quinolinolato)aluminum (hereinafter, abbreviated as “BAlq”) was vacuum-deposited with a thickness of 10 nm for a hole blocking layer, and tris(8-quinolinol)aluminum (hereinafter, abbreviated as “Alq3”) was formed with a thickness of 40 nm for an electron transport layer. Thereafter, LiF as halogenated alkali metal was deposited with a thickness of 0.2 nm for an electron injection layer, and then Al was deposited with a thickness of 150 nm to be used as a cathode. In this way, an organic electronic light emitting element was manufactured.

### Example 4-186 to Example 4-196 Red Organic Light Emitting Element (Phosphorescent Host)

An organic electronic light emitting element was manufactured by the same method as in Example 4-185 except that, instead of compound 2-41-4 of the present invention, one of compounds 2-42-4 to 2-52-4 listed on table 4-6 was used as a phosphorescent host material for a light emitting layer.

### Comparative Example 4-5

An organic electronic light emitting element was manufactured by the same method as in Example 4-185 except that, instead of compound 2-41-4 of the present invention, comparative compound A [4,4′-N,N′-dicarbazole-biphenyl (CBP)] above was used as a phosphorescent host material for a light emitting layer.

### Comparative Example 4-6

An organic electronic light emitting element was manufactured by the same method as in Example 4-185 except that, instead of compound 2-41-4 of the present invention, comparative compound B above was used as a phosphorescent host material for a light emitting layer.

### Comparative Example 4-7

An organic electronic light emitting element was manufactured by the same method as in Example 4-185 except that, instead of compound 2-41-4 of the present invention, comparative compound C above was used as a phosphorescent host material for a light emitting layer.

### Comparative Example 4-8

An organic electronic light emitting element was manufactured by the same method as in Example 4-185 except that, instead of compound 2-41-4 of the present invention, comparative compound D above was used as a phosphorescent host material for a light emitting layer.

A forward bias DC voltage was applied to the organic electronic light emitting elements manufactured in Examples 4-185 to 4-196 and Comparative Examples 4-5 to 4-8 to measure electro-luminescence (EL) characteristics thereof by PR-650 (Photoresearch), and the T95 lifetime was measured by lifetime measuring equipments (Mcscience) at reference brightness of 2500 cd/m². Table 4-6 below shows the manufacture of elements and evaluation results thereof.

TABLE 4-6

| | | | Current | Brightness | | Lifetime | CIE |
|---|---|---|---|---|---|---|---|
| | Compound | Voltage | Density | (cd/m2) | Efficiency | T(95) | (x, y) |
| Comparative | Compound | 6.2 | 39.7 | 2500.0 | 6.3 | 53.3 | (0.66, |
| Example(4-5) | (A) | | | | | | 0.35) |
| Comparative | Compound | 5.7 | 32.5 | 2500.0 | 7.7 | 97.2 | (0.66, |
| Example(4-6) | (B) | | | | | | 0.35) |
| Comparative | Compound | 5.8 | 34.8 | 2500.0 | 7.2 | 91.8 | (0.66, |
| Example(4-7) | (C) | | | | | | 0.35) |
| Comparative | Compound | 5.9 | 34.7 | 2500.0 | 7.2 | 93.3 | (0.66, |
| Example(4-8) | (D) | | | | | | 0.35) |
| Example(4-185) | Compound | 5.1 | 27.2 | 2500.0 | 9.2 | 119.0 | (0.66, |
| | (2-41-4) | | | | | | 0.37) |
| Example(4-186) | Compound | 5.0 | 28.3 | 2500.0 | 8.8 | 124.1 | (0.66, |
| | (2-42-4) | | | | | | 0.35) |
| Example(4-187) | Compound | 5.2 | 28.6 | 2500.0 | 8.7 | 122.3 | (0.66, |
| | (2-43-4) | | | | | | 0.35) |
| Example(4-188) | Compound | 5.3 | 29.3 | 2500.0 | 8.5 | 116.8 | (0.66, |
| | (2-44-4) | | | | | | 0.36) |
| Example(4-189) | Compound | 5.1 | 28.8 | 2500.0 | 8.7 | 111.2 | (0.66, |
| | (2-45-4) | | | | | | 0.35) |
| Example(4-190) | Compound | 5.0 | 27.8 | 2500.0 | 9.0 | 113.7 | (0.66, |
| | (2-46-4) | | | | | | 0.35) |
| Example(4-191) | Compound | 5.2 | 27.8 | 2500.0 | 9.0 | 111.4 | (0.66, |
| | (2-47-4) | | | | | | 0.35) |
| Example(4-192) | Compound | 5.2 | 27.2 | 2500.0 | 9.2 | 103.5 | (0.66, |
| | (2-48-4) | | | | | | 0.35) |
| Example(4-193) | Compound | 5.2 | 26.4 | 2500.0 | 9.5 | 115.4 | (0.66, |
| | (2-49-4) | | | | | | 0.35) |
| Example(4-194) | Compound | 5.4 | 26.4 | 2500.0 | 9.5 | 111.5 | (0.66, |
| | (2-50-4) | | | | | | 0.34) |
| Example(4-195) | Compound | 5.2 | 27.0 | 2500.0 | 9.3 | 121.3 | (0.66, |
| | (2-51-4) | | | | | | 0.35) |
| Example(4-196) | Compound | 5.2 | 28.7 | 2500.0 | 8.7 | 115.7 | (0.66, |
| | (2-52-4) | | | | | | 0.35) |

As can be seen from the results on table 4-5 and table 4-6, the organic electronic light emitting elements using the materials for the organic electronic light emitting element of the present invention as a phosphorescent host showed a low driving voltage, high light emitting efficiency, and a long lifetime.

In other words, comparative compounds B, C, and D having bis-carbazole as a core showed excellent element results compared with comparative compound A, which is CBP generally used as a host material, and the compounds of the present invention having carbazole linked to carboline showed the best results in view of a driving voltage, efficiency, and a lifetime, compared with comparative compounds B, C, and D.

The compound according to the present invention has a bipolar since it is composed of carbazole and carboline. Therefore, it is considered that the compounds of the present invention can raise the charge balance in the light emitting layer compared with those in comparative compounds B, C, and D, leading to an increase in efficiency, and shows less hole accumulation in the light emitting layer compared with comparative compounds B, C, and D, leading to a long lifetime (In the driving of OLED, holes generally have 1000-fold higher mobility than electrons).

In addition, the compounds according to the present invention have similar T1 values to comparative compounds B, C, and D, but show lower LUMO values, and resultantly, it is considered that the compounds of the present invention may easily receive electrons from the electron transport layer, leading to a low driving voltage and excellent thermal stability (thermal damage due to a high driving voltage).

### III. Manufacture and Test of Green Organic Light Emitting Element (Phosphorescent Host)

### Example 4-197 Green Organic Light Emitting Element (Phosphorescent Host)

An organic electronic light emitting element was manufactured by an ordinary method using the compound obtained through synthesis as a host material for a light emitting layer. First, a film of N¹-(naphthalen-2-yl)-N⁴,N⁴-bis(4-(naphthalen-2-yl(phenyl)amino)phenyl)-N¹-phenylbenzene-1,4-diamine (hereinafter, abbreviated as “2-TNATA”) as a hole injection layer was vacuum-deposited with a thickness of 60 nm on an ITO layer (anode) formed on a galas substrate. Then, 4,4-bis[N-(1-naphthyl)-N-phenylamino]biphenyl (hereinafter, abbreviated as “-NPD”) as a hole transport compound was vacuum-deposited on the hole injection layer to form a hole transport layer with a thickness of 60 nm. Subsequently, a light emitting layer with a thickness of 30 nm was formed on the hole transport layer by doping an upper portion of the hole transport layer with the compound 3-56-4 of the present invention as a host and Ir(ppy)₃ [tris(2-phenylpyridine)-iridium] as a dopant at a weight ratio of 95:5. Then, (1.1′-bisphenyl)-4-olato)bis(2-methyl-8-quinolinolato)aluminum (hereinafter, abbreviated as “BAlq”) was vacuum-deposited with a thickness of 10 nm for a hole blocking layer, and tris(8-quinolinol)aluminum (hereinafter, abbreviated as “Alq3”) was formed with a thickness of 40 nm for an electron injection layer. Thereafter, LiF as halogenated alkali metal was deposited with a thickness of 0.2 nm, and subsequently Al was deposited with a thickness of 150 nm, thereby using this Al/LiF as a cathode. In this way, an organic electronic light emitting element was manufactured.

### Example 4-198 to Example 4-250 Green Organic Light Emitting Element (Phosphorescent Host)

An organic electronic light emitting element was manufactured by the same method as in Example 197 except that, instead of compound 3-56-4 of the present invention, one of compounds 3-60-4, and 3-69-4 to 3-112-4 of the present invention listed on table 4-7 below was used as a phosphorescent host material for a light emitting layer.

### Comparative Example 4-9

An organic electronic light emitting element was manufactured by the same method as in Example 4-197 except that, instead of compound 3-56-4 of the present invention, comparative compound E below was used as a phosphorescent host material for a light emitting layer.

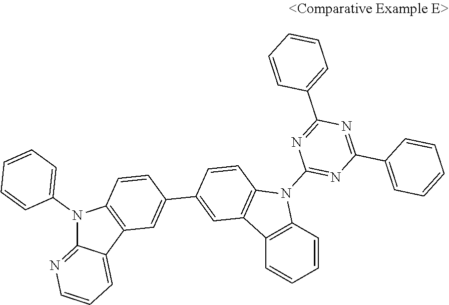 [SMILES: c1ccc(-c2nc(-c3ccccc3)nc(-n3c4ccccc4c4cc(-c5ccc6c(c5)c5cccnc5n6-c5ccccc5)ccc43)n2)cc1]

A forward bias DC voltage was applied to the organic electronic light emitting elements manufactured in Examples 4-197 to 4-250 and Comparative Example 4-9 to measure electro-luminescence (EL) characteristics thereof by PR-650 (Photoresearch), and the T95 lifetime was measured by lifetime measuring equipments (Mcscience) at reference brightness of 5000 cd/m². Table 4-7 below shows the manufacture of elements and evaluation results thereof.

TABLE 4-7

| | | | Current | Brightness | | Lifetime | CIE |
|---|---|---|---|---|---|---|---|
| | Compound | Voltage | Density | (cd/m2) | Efficiency | T(95) | (x, y) |
| Comparative | Compound | 5.1 | 16.2 | 5000.0 | 30.8 | 97.9 | (0.31, |
| Example(4-9) | (E) | | | | | | 0.60) |
| Example(4-197) | Compound | 5.0 | 14.3 | 5000.0 | 34.9 | 106.2 | (0.33, |
| | (3-56-4) | | | | | | 0.61) |
| Example(4-198) | Compound | 5.0 | 15.4 | 5000.0 | 32.5 | 129.2 | (0.31, |
| | (3-60-4) | | | | | | 0.60) |
| Example(4-206) | Compound | 5.1 | 15.9 | 5000.0 | 31.5 | 120.3 | (0.32, |
| | (3-68-4) | | | | | | 0.61) |
| Example(4-207) | Compound | 5.1 | 16.0 | 5000.0 | 31.2 | 122.7 | (0.33, |
| | (3-69-4) | | | | | | 0.60) |
| Example(4-208) | Compound | 5.1 | 16.1 | 5000.0 | 31.2 | 124.7 | (0.32, |
| | (3-70-4) | | | | | | 0.61) |
| Example(4-209) | Compound | 5.0 | 15.2 | 5000.0 | 32.8 | 120.9 | (0.31, |
| | (3-71-4) | | | | | | 0.60) |
| Example(4-210) | Compound | 5.0 | 15.2 | 5000.0 | 32.9 | 126.8 | (0.31, |
| | (3-72-4) | | | | | | 0.61) |
| Example(4-211) | Compound | 5.0 | 15.3 | 5000.0 | 32.7 | 123.0 | (0.31, |
| | (3-73-4) | | | | | | 0.60) |
| Example(4-212) | Compound | 5.0 | 16.0 | 5000.0 | 31.2 | 123.1 | (0.33, |
| | (3-74-4) | | | | | | 0.61) |
| Example(4-213) | Compound | 5.0 | 15.7 | 5000.0 | 31.8 | 127.1 | (0.30, |
| | (3-75-4) | | | | | | 0.60) |
| Example(4-214) | Compound | 5.0 | 15.6 | 5000.0 | 32.0 | 125.0 | (0.31, |
| | (3-76-4) | | | | | | 0.61) |
| Example(4-215) | Compound | 4.9 | 16.1 | 5000.0 | 31.1 | 125.2 | (0.31, |
| | (3-77-4) | | | | | | 0.60) |
| Example(4-216) | Compound | 5.0 | 15.2 | 5000.0 | 32.9 | 126.9 | (0.33, |
| | (3-78-4) | | | | | | 0.61) |
| Example(4-217) | Compound | 5.0 | 15.3 | 5000.0 | 32.7 | 126.9 | (0.32, |
| | (3-79-4) | | | | | | 0.61) |
| Example(4-218) | Compound | 5.0 | 15.9 | 5000.0 | 31.4 | 121.5 | (0.33, |
| | (3-80-4) | | | | | | 0.60) |
| Example(4-219) | Compound | 5.0 | 16.1 | 5000.0 | 31.1 | 124.7 | (0.31, |
| | (3-81-4) | | | | | | 0.61) |
| Example(4-220) | Compound | 5.0 | 15.7 | 5000.0 | 31.9 | 124.0 | (0.31, |
| | (3-82-4) | | | | | | 0.60) |
| Example(4-221) | Compound | 5.1 | 15.4 | 5000.0 | 32.5 | 125.0 | (0.33, |
| | (3-83-4) | | | | | | 0.61) |
| Example(4-222) | Compound | 5.0 | 15.3 | 5000.0 | 32.6 | 125.8 | (0.32, |
| | (3-84-4) | | | | | | 0.61) |
| Example(4-223) | Compound | 5.1 | 15.5 | 5000.0 | 32.2 | 126.9 | (0.33, |
| | (3-85-4) | | | | | | 0.60) |
| Example(4-224) | Compound | 5.1 | 15.9 | 5000.0 | 31.5 | 126.4 | (0.32, |
| | (3-86-4) | | | | | | 0.61) |
| Example(4-225) | Compound | 5.1 | 15.2 | 5000.0 | 32.9 | 123.8 | (0.31, |
| | (3-87-4) | | | | | | 0.60) |
| Example(4-226) | Compound | 4.9 | 15.7 | 5000.0 | 31.8 | 120.5 | (0.31, |
| | (3-88-4) | | | | | | 0.61) |
| Example(4-227) | Compound | 4.9 | 15.3 | 5000.0 | 32.7 | 126.8 | (0.31, |
| | (3-89-4) | | | | | | 0.60) |
| Example(4-228) | Compound | 4.9 | 15.7 | 5000.0 | 31.9 | 125.0 | (0.33, |
| | (3-90-4) | | | | | | 0.61) |
| Example(4-229) | Compound | 5.0 | 15.3 | 5000.0 | 32.7 | 129.5 | (0.30, |
| | (3-91-4) | | | | | | 0.60) |
| Example(4-230) | Compound | 5.1 | 15.9 | 5000.0 | 31.4 | 128.5 | (0.31, |
| | (3-92-4) | | | | | | 0.61) |
| Example(4-231) | Compound | 5.1 | 15.5 | 5000.0 | 32.3 | 125.4 | (0.31, |
| | (3-93-4) | | | | | | 0.60) |
| Example(4-232) | Compound | 5.1 | 16.1 | 5000.0 | 31.0 | 127.3 | (0.33, |
| | (3-94-4) | | | | | | 0.61) |
| Example(4-233) | Compound | 4.9 | 15.5 | 5000.0 | 32.3 | 128.9 | (0.32, |
| | (3-95-4) | | | | | | 0.61) |
| Example(4-234) | Compound | 5.1 | 15.7 | 5000.0 | 31.9 | 122.7 | (0.33, |
| | (3-96-4) | | | | | | 0.60) |
| Example(4-235) | Compound | 5.1 | 15.6 | 5000.0 | 32.0 | 126.7 | (0.32, |
| | (3-97-4) | | | | | | 0.61) |
| Example(4-236) | Compound | 4.9 | 15.5 | 5000.0 | 32.2 | 123.8 | (0.31, |
| | (3-98-4) | | | | | | 0.60) |
| Example(4-237) | Compound | 4.9 | 15.7 | 5000.0 | 31.9 | 129.1 | (0.31, |
| | (3-99-4) | | | | | | 0.61) |
| Example(4-238) | Compound | 5.0 | 15.7 | 5000.0 | 31.9 | 123.2 | (0.31, |
| | (3-100-4) | | | | | | 0.60) |
| Example(4-239) | Compound | 5.0 | 15.4 | 5000.0 | 32.6 | 129.0 | (0.33, |
| | (3-101-4) | | | | | | 0.61) |
| Example(4-240) | Compound | 4.9 | 15.3 | 5000.0 | 32.7 | 121.4 | (0.30, |
| | (3-102-4) | | | | | | 0.60) |
| Example(4-241) | Compound | 5.1 | 16.0 | 5000.0 | 31.2 | 124.5 | (0.31, |
| | (3-103-4) | | | | | | 0.61) |
| Example(4-242) | Compound | 5.0 | 15.2 | 5000.0 | 32.9 | 125.2 | (0.31, |
| | (3-104-4) | | | | | | 0.60) |
| Example(4-243) | Compound | 5.1 | 15.6 | 5000.0 | 32.1 | 125.7 | (0.33, |
| | (3-105-4) | | | | | | 0.61) |
| Example(4-244) | Compound | 5.0 | 16.0 | 5000.0 | 31.3 | 128.2 | (0.32, |
| | (3-106-4) | | | | | | 0.61) |
| Example(4-245) | Compound | 5.0 | 15.7 | 5000.0 | 31.8 | 125.2 | (0.33, |
| | (3-107-4) | | | | | | 0.60) |
| Example(4-246) | Compound | 5.1 | 15.7 | 5000.0 | 31.9 | 120.8 | (0.32, |
| | (3-108-4) | | | | | | 0.61) |
| Example(4-247) | Compound | 5.1 | 15.6 | 5000.0 | 32.1 | 125.1 | (0.31, |
| | (3-109-4) | | | | | | 0.60) |
| Example(4-248) | Compound | 5.1 | 15.8 | 5000.0 | 31.7 | 123.0 | (0.33, |
| | (3-110-4) | | | | | | 0.61) |
| Example(4-249) | Compound | 5.1 | 15.7 | 5000.0 | 31.9 | 129.5 | (0.30, |
| | (3-111-4) | | | | | | 0.60) |
| Example(4-250) | Compound | 5.0 | 15.6 | 5000.0 | 32.0 | 127.6 | (0.31, |
| | (3-112-4) | | | | | | 0.61) |

As can be seen from the results of Table 4-7, the organic electronic light emitting elements using the materials for an organic electronic light emitting element of the present invention as a phosphorescent host showed more improved results than Comparative Compound.

In other words, from the comparative results between comparative compound E in which carboline having N substituted at the α-position and carbazole are substituted with 3-3 and compound 3-56 of the present invention in which carboline having N substituted at the β-position and carbazole are substituted with 3-3, it can be verified that the driving voltage and lifetime were similar therebetween but the efficiency was improved in the present invention.

When N is introduced at the β-position on carboline, the LUMO energy level is higher due to weak electron acceptor characteristics compared with the introduction at the α-position, and the HOMO energy level is similar since the HOMO level is dependent on the carbazole unit. Finally, the introduction of N at the the β-position has a wider energy band gap than the introduction of N at the α-position. Due to this band gap difference, comparative compound E having the substitution at the α-position emits light in a longer wavelength region compared with compound 3-56 having the substitution at the β-position, and thus, when compound 3-56-4 emits light in a shorter wavelength region was used as a green host, the efficiency was more improved.

Whereas, inventive compound 3-60 having N substituted at the γ-position and inventive compound 3-68 to 3-112 having N substituted at the δ-position show no band gap difference compared with comparative compound E, and thus similar efficiency but excellent lifetimes were verified. It is considered that Cz-γCb and Cz-δCb showed higher Tg and Tm than Cz-αCb, leading to increased thermal stability, which showed such results.

That is, it can be seen, on the basis of the above element results, that the change in the position of the N atom on the carboline unit changes in the energy level, and thus significantly changes the characteristics of elements.

In addition, the characteristics of elements have been described in view of a light emitting layer from the foregoing evaluation results of the manufacture of elements, but the materials used for a light emitting layer may be used alone or in a mixture with other materials, for the foregoing organic material layer for an organic electronic element, such as an an electron injection layer, an electron injection layer, a hole injection layer, a hole transport layer, and an auxiliary light emitting layer. Therefore, for the foregoing reasons, the compounds of the present invention may be used alone or in a mixture with other materials, for the other layers for the organic material layer excluding the light emitting layer, for example, an electron injection layer, a hole injection layer, a hole transport layer, and an auxiliary light emitting layer.

Although exemplary embodiments of the present invention have been described for illustrative purposes, a person skilled in the art will appreciate that various modifications, additions, and substitutions are possible, without departing from the scope and spirit of the invention as disclosed in the accompanying claims. Therefore, the embodiment disclosed in the present invention is intended to illustrate the scope of the technical idea of the present invention, and the scope of the present invention is not limited by the embodiment. The scope of the present invention shall be construed on the basis of the accompanying claims, and it shall be construed that all of the technical ideas included within the scope equivalent to the claims belong to the present invention.

### EXPLANATION OF NUMERICAL REFERENCES

100: organic electronic element
110: substrate
120: first electrode
130: hole injection layer
140: hole transport layer
141: buffer layer
150: light emitting layer
151: auxiliary light emitting layer
160: electron transport layer
170: electron injection layer
180: second electrode

1. A compound represented by one of Formulas 1-1, 2-1, or 3-1: 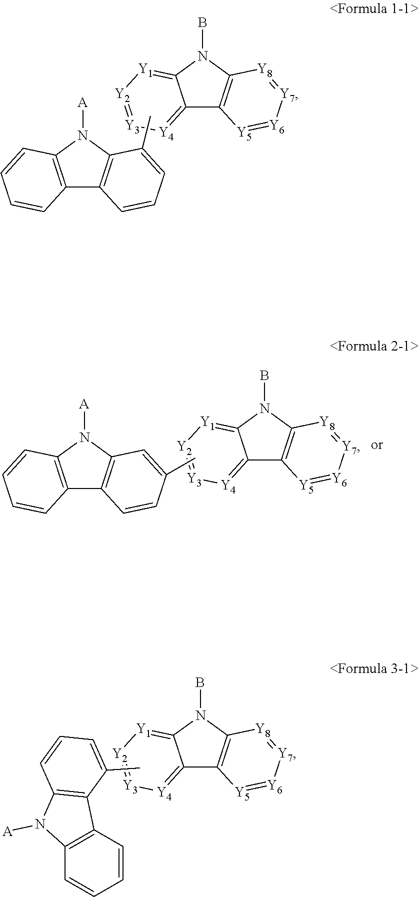
wherein in Formulas 1-1, 2-1, or 3-1,
A and B each are independently selected from the group consisting of a C₆-C₆₀ aryl group, a fluorenyl group, a C₂-C₆₀ heterocyclic group containing at least one heteroatom of O, N, S, Si, and P, a fused ring group of a C₃-C₆₀ aliphatic group and a C₆-C₆₀ aromatic group, a C₁-C₅₀ alkyl group, a C₂-C₂₀ alkenyl group, a C₂-C₂₀ alkynyl group, C₁-C₃₀ alkoxyl group, a C₆-C₃₀ aryloxy group, and -L′-N(Rₐ)(R_{b});
L′ is selected from the group consisting of a single bond, a C₆-C₆₀ arylene group, a fluorenyl group, a fused ring group of a C₃-C₆₀ aliphatic group and a C₆-C₆₀ aromatic group, and a C₂-C₆₀ heterocyclic group;
Rₐ and R_{b} each are independently selected from the group consisting of a C₆-C₆₀ aryl group, a fluorenylene group, a fused ring group of a C₃-C₆₀ aliphatic group and a C₆-C₆₀ aromatic group, and a C₂-C₆₀ heterocyclic group containing at least one heteroatom of O, N, S, Si, and P;
Y₁ to Y₈ each are independently CR or N, and at least one of Y₅ to Y₈ is N;
at least one of Rs is linked to adjacent carbazole, and R that is not linked thereto is hydrogen;
the aryl group, fluorenyl group, heterocyclic group, fused ring group, alkyl group, alkenyl group, alkoxyl group, aryloxy group, arylene group, and fluorenylene group each may be substituted with at least one substituent selected from the group consisting of deuterium, halogen, a silane group, a siloxane group, a boron group, a germanium group, a cyano group, a nitro group, a C₁-C₂₀ alkylthio group, a C₁-C₂₀ alkoxyl group, a C₁-C₂₀ alkyl group, a C₂-C₂₀ alkenyl group, a C₂-C₂₀ alkynyl group, a C₆-C₂₀ aryl group, a C₆-C₂₀ aryl group substituted with deuterium, a fluorenyl group, a C₂-C₂₀ heterocyclic group, a C₃-C₂₀ cycloalkyl group, a C₇-C₂₀ arylalkyl group, and a C₈-C₂₀ arylalkenyl group.

2. The compound of claim 1, wherein the compound is represented by one of the formulas below: 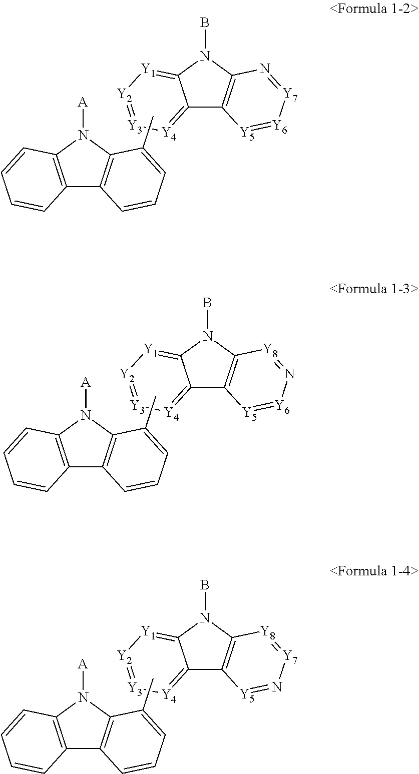 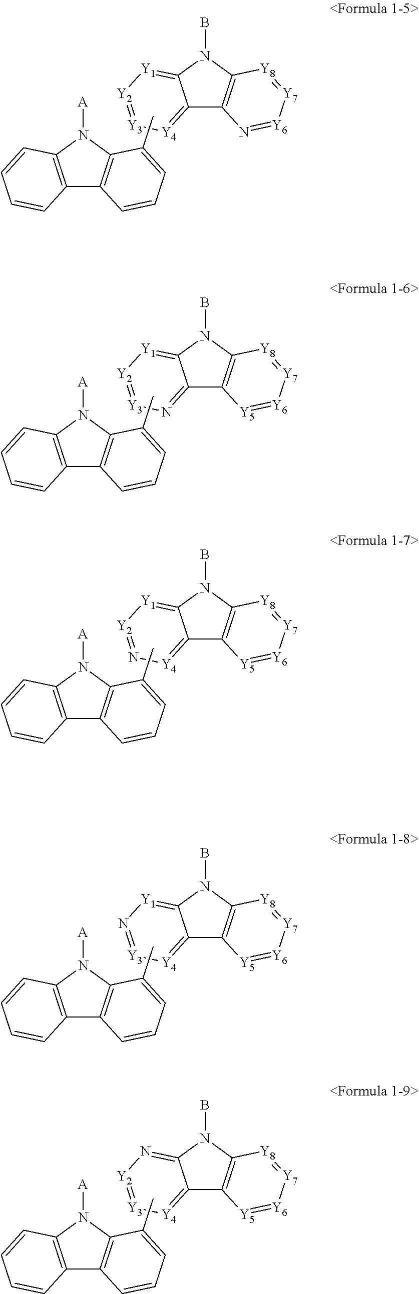
wherein in Formulas 1-2 to 1-9, Y₁ to Y₈ and A and B are identical to Y₁ to Y₈ and A and B defined in Formula 1-1.

3. The compound of claim 1, wherein the compound is represented by one of the formulas below:

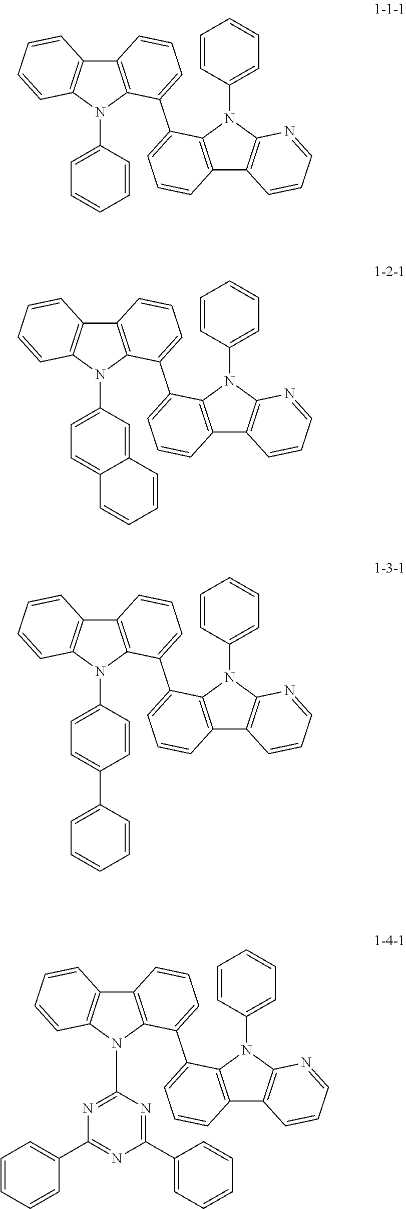 [SMILES: c1ccc(-n2c3ccccc3c3cccc(-c4cccc5c6cccnc6n(-c6ccccc6)c45)c32)cc1 ; c1ccc(-n2c3ncccc3c3cccc(-c4cccc5c6ccccc6n(-c6ccc7ccccc7c6)c45)c32)cc1 ; c1ccc(-c2ccc(-n3c4ccccc4c4cccc(-c5cccc6c7cccnc7n(-c7ccccc7)c56)c43)cc2)cc1 ; c1ccc(-c2nc(-c3ccccc3)nc(-n3c4ccccc4c4cccc(-c5cccc6c7cccnc7n(-c7ccccc7)c56)c43)n2)cc1]

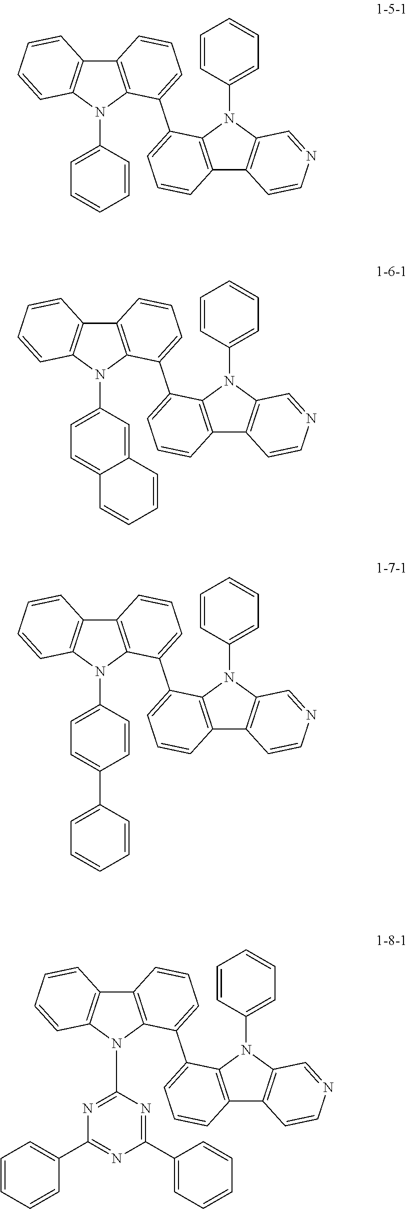 [SMILES: c1ccc(-n2c3ccccc3c3cccc(-c4cccc5c6ccncc6n(-c6ccccc6)c45)c32)cc1 ; c1ccc(-n2c3cnccc3c3cccc(-c4cccc5c6ccccc6n(-c6ccc7ccccc7c6)c45)c32)cc1 ; c1ccc(-c2ccc(-n3c4ccccc4c4cccc(-c5cccc6c7ccncc7n(-c7ccccc7)c56)c43)cc2)cc1 ; c1ccc(-c2nc(-c3ccccc3)nc(-n3c4ccccc4c4cccc(-c5cccc6c7ccncc7n(-c7ccccc7)c56)c43)n2)cc1]

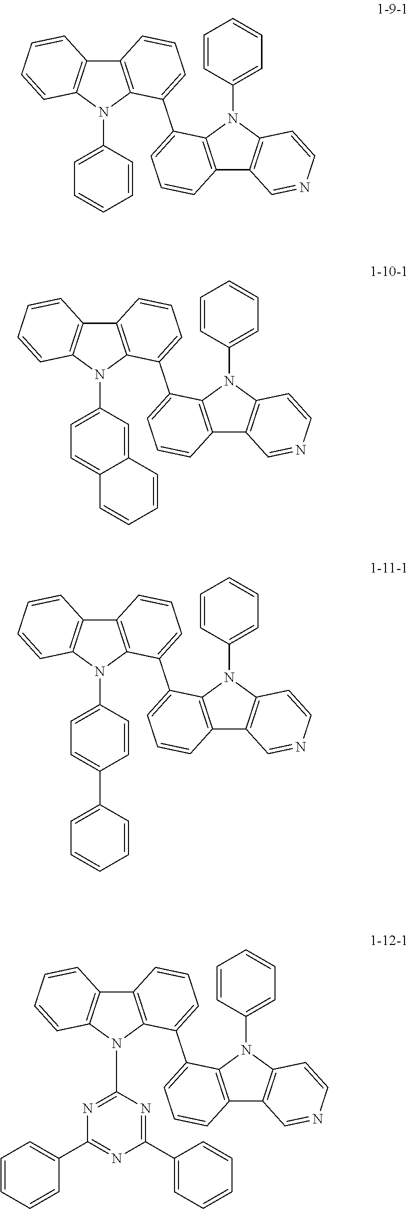 [SMILES: c1ccc(-n2c3ccccc3c3cccc(-c4cccc5c6cnccc6n(-c6ccccc6)c45)c32)cc1 ; c1ccc(-n2c3ccncc3c3cccc(-c4cccc5c6ccccc6n(-c6ccc7ccccc7c6)c45)c32)cc1 ; c1ccc(-c2ccc(-n3c4ccccc4c4cccc(-c5cccc6c7cnccc7n(-c7ccccc7)c56)c43)cc2)cc1 ; c1ccc(-c2nc(-c3ccccc3)nc(-n3c4ccccc4c4cccc(-c5cccc6c7cnccc7n(-c7ccccc7)c56)c43)n2)cc1]

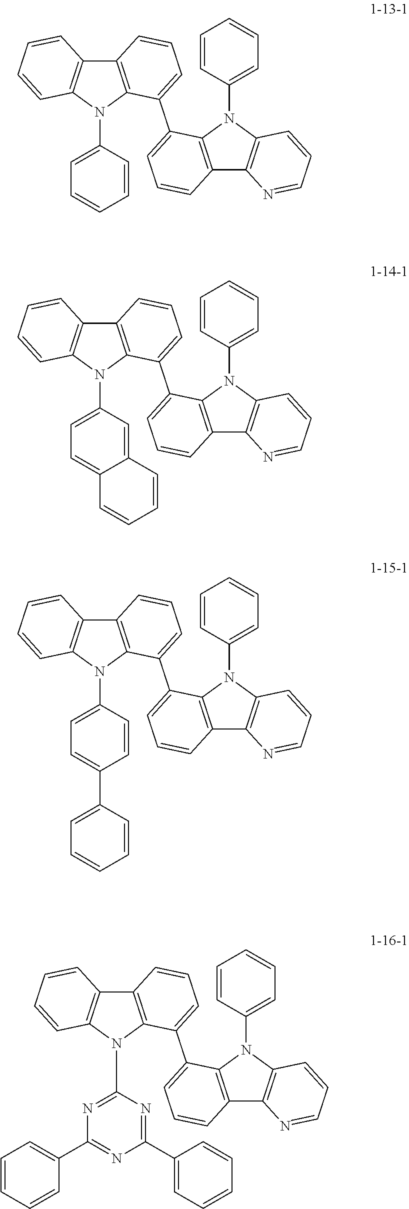 [SMILES: c1ccc(-n2c3ccccc3c3cccc(-c4cccc5c6ncccc6n(-c6ccccc6)c45)c32)cc1 ; c1ccc(-n2c3cccnc3c3cccc(-c4cccc5c6ccccc6n(-c6ccc7ccccc7c6)c45)c32)cc1 ; c1ccc(-c2ccc(-n3c4ccccc4c4cccc(-c5cccc6c7ncccc7n(-c7ccccc7)c56)c43)cc2)cc1 ; c1ccc(-c2nc(-c3ccccc3)nc(-n3c4ccccc4c4cccc(-c5cccc6c7ncccc7n(-c7ccccc7)c56)c43)n2)cc1]

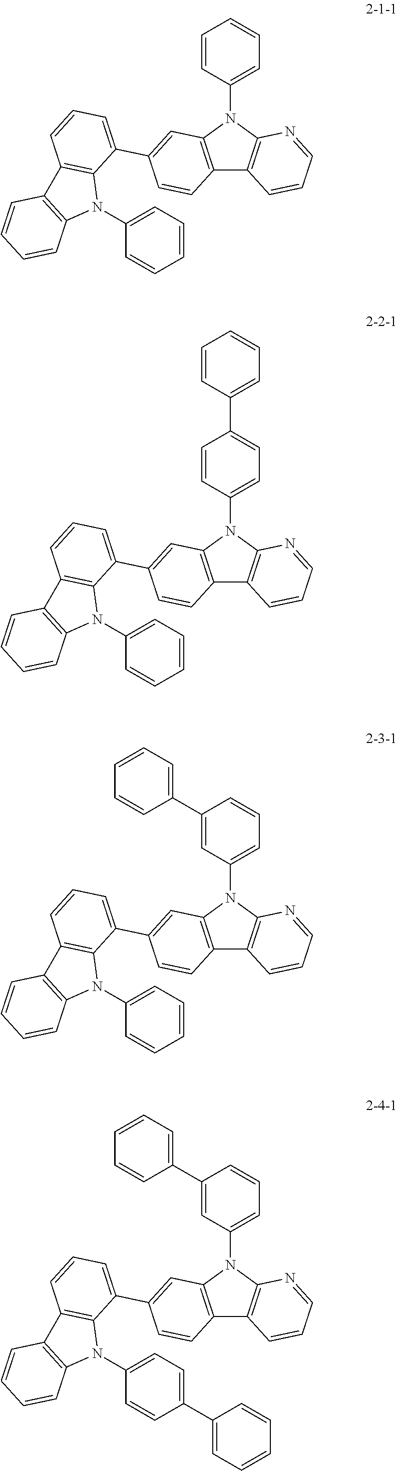 [SMILES: c1ccc(-n2c3cc(-c4cccc5c6ccccc6n(-c6ccccc6)c45)ccc3c3cccnc32)cc1 ; c1ccc(-c2ccc(-n3c4cc(-c5cccc6c7ccccc7n(-c7ccccc7)c56)ccc4c4cccnc43)cc2)cc1 ; c1ccc(-c2cccc(-n3c4cc(-c5cccc6c7ccccc7n(-c7ccccc7)c56)ccc4c4cccnc43)c2)cc1 ; c1ccc(-c2ccc(-n3c4ccccc4c4cccc(-c5ccc6c7cccnc7n(-c7cccc(-c8ccccc8)c7)c6c5)c43)cc2)cc1]

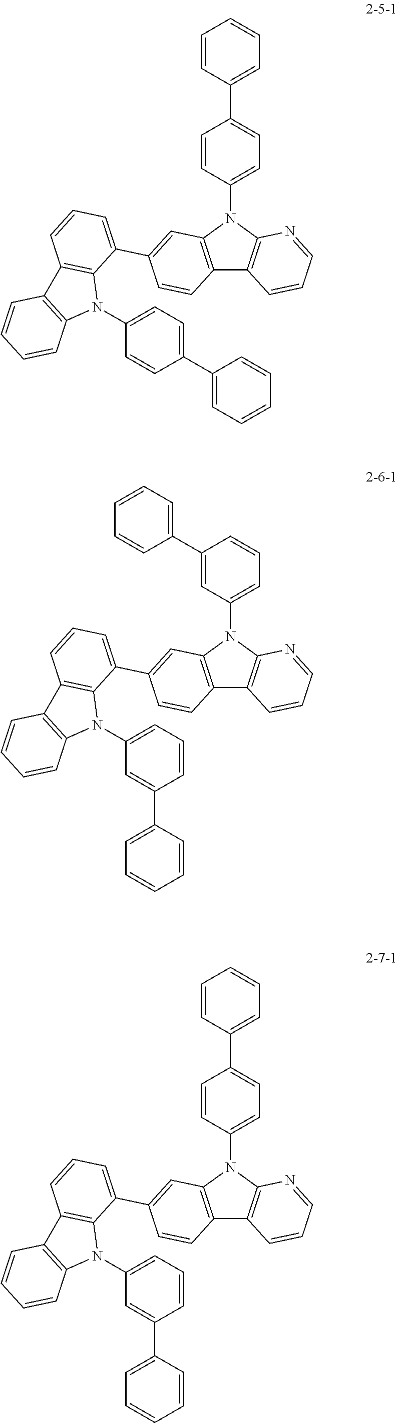 [SMILES: c1ccc(-c2ccc(-n3c4cc(-c5cccc6c7ccccc7n(-c7ccc(-c8ccccc8)cc7)c56)ccc4c4cccnc43)cc2)cc1 ; c1ccc(-c2cccc(-n3c4cc(-c5cccc6c7ccccc7n(-c7cccc(-c8ccccc8)c7)c56)ccc4c4cccnc43)c2)cc1 ; c1ccc(-c2ccc(-n3c4cc(-c5cccc6c7ccccc7n(-c7cccc(-c8ccccc8)c7)c56)ccc4c4cccnc43)cc2)cc1]

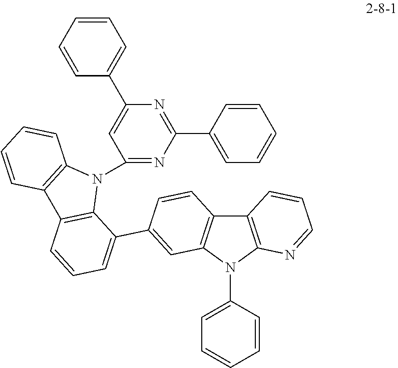 [SMILES: c1ccc(-c2cc(-n3c4ccccc4c4cccc(-c5ccc6c7cccnc7n(-c7ccccc7)c6c5)c43)nc(-c3ccccc3)n2)cc1]

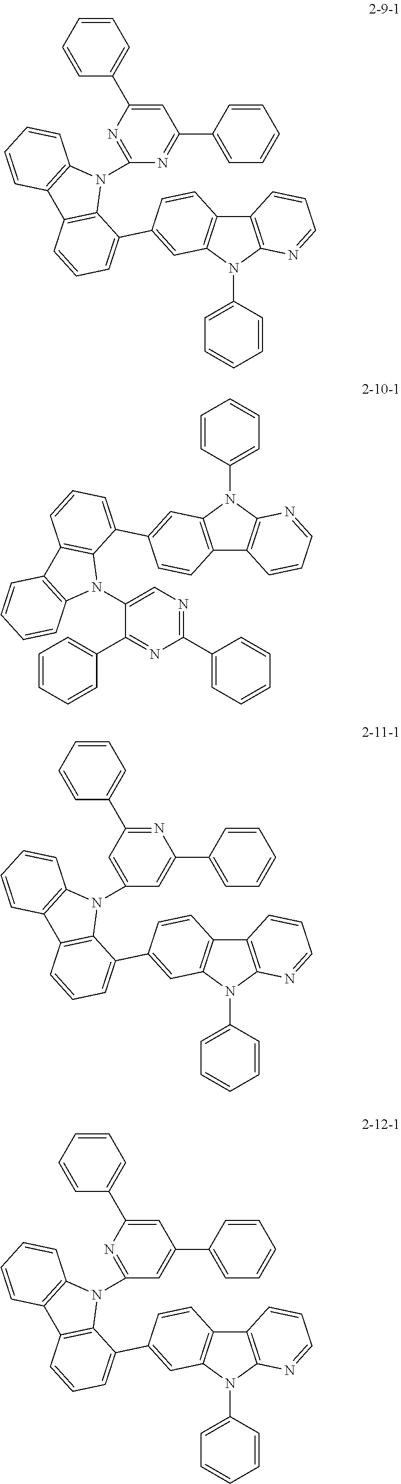

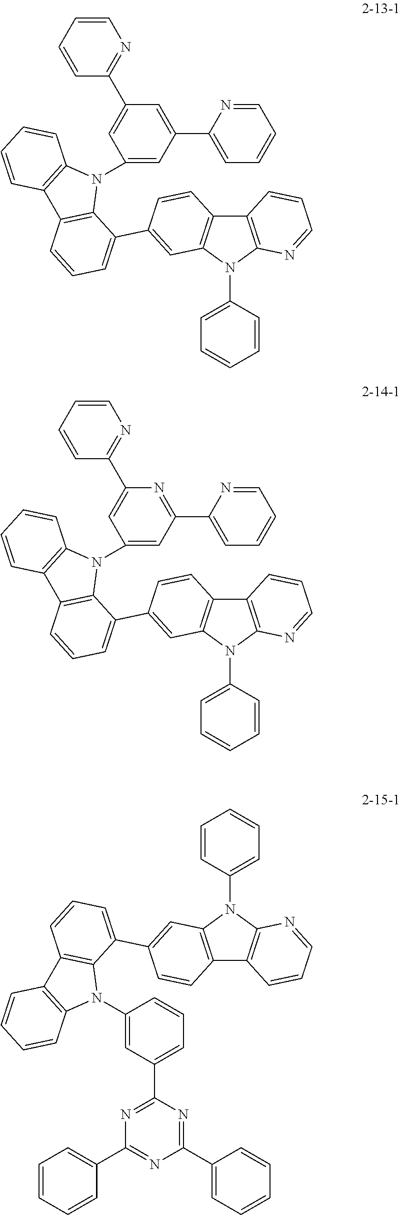 [SMILES: c1ccc(-n2c3cc(-c4cccc5c6ccccc6n(-c6cc(-c7ccccn7)cc(-c7ccccn7)c6)c45)ccc3c3cccnc32)cc1 ; c1ccc(-n2c3cc(-c4cccc5c6ccccc6n(-c6cc(-c7ccccn7)nc(-c7ccccn7)c6)c45)ccc3c3cccnc32)cc1 ; c1ccc(-c2nc(-c3ccccc3)nc(-c3cccc(-n4c5ccccc5c5cccc(-c6ccc7c8cccnc8n(-c8ccccc8)c7c6)c54)c3)n2)cc1]

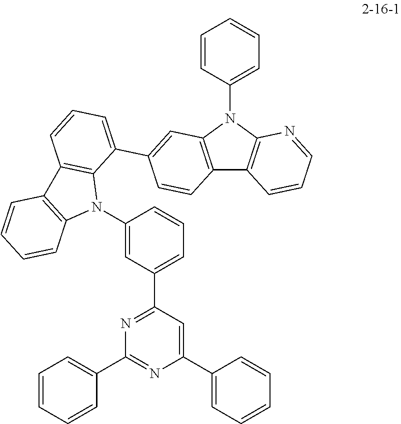 [SMILES: c1ccc(-c2cc(-c3cccc(-n4c5ccccc5c5cccc(-c6ccc7c8cccnc8n(-c8ccccc8)c7c6)c54)c3)nc(-c3ccccc3)n2)cc1]

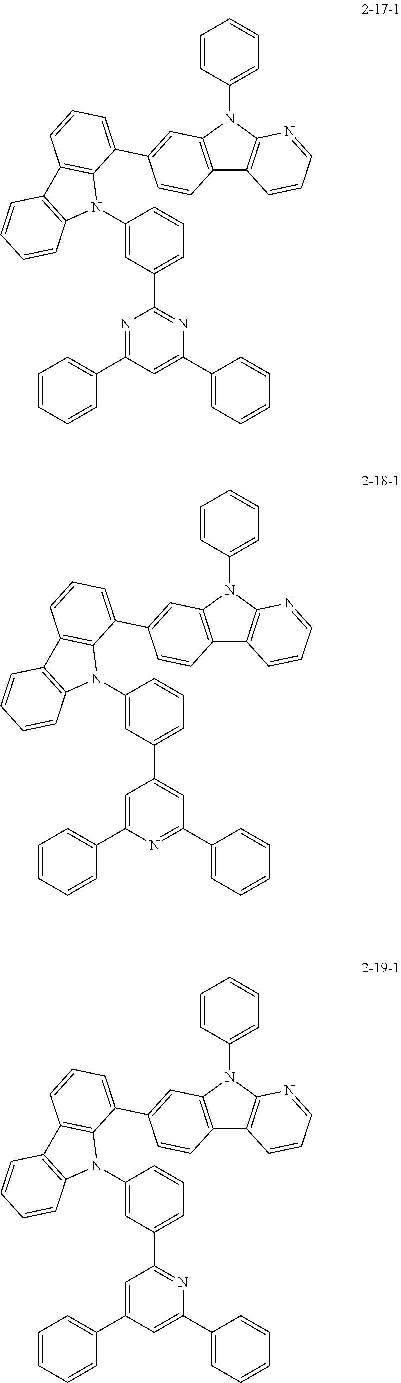 [SMILES: c1ccc(-c2cc(-c3ccccc3)nc(-c3cccc(-n4c5ccccc5c5cccc(-c6ccc7c8cccnc8n(-c8ccccc8)c7c6)c54)c3)n2)cc1 ; c1ccc(-c2cc(-c3cccc(-n4c5ccccc5c5cccc(-c6ccc7c8cccnc8n(-c8ccccc8)c7c6)c54)c3)cc(-c3ccccc3)n2)cc1 ; c1ccc(-c2cc(-c3ccccc3)nc(-c3cccc(-n4c5ccccc5c5cccc(-c6ccc7c8cccnc8n(-c8ccccc8)c7c6)c54)c3)c2)cc1]

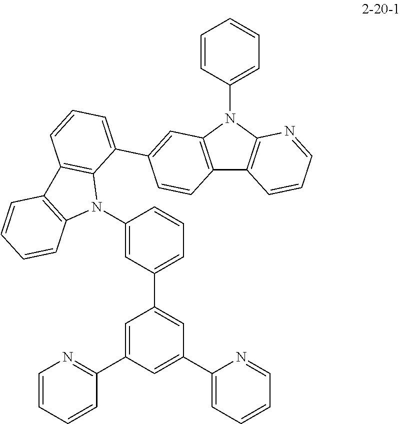 [SMILES: c1ccc(-n2c3cc(-c4cccc5c6ccccc6n(-c6cccc(-c7cc(-c8ccccn8)cc(-c8ccccn8)c7)c6)c45)ccc3c3cccnc32)cc1]

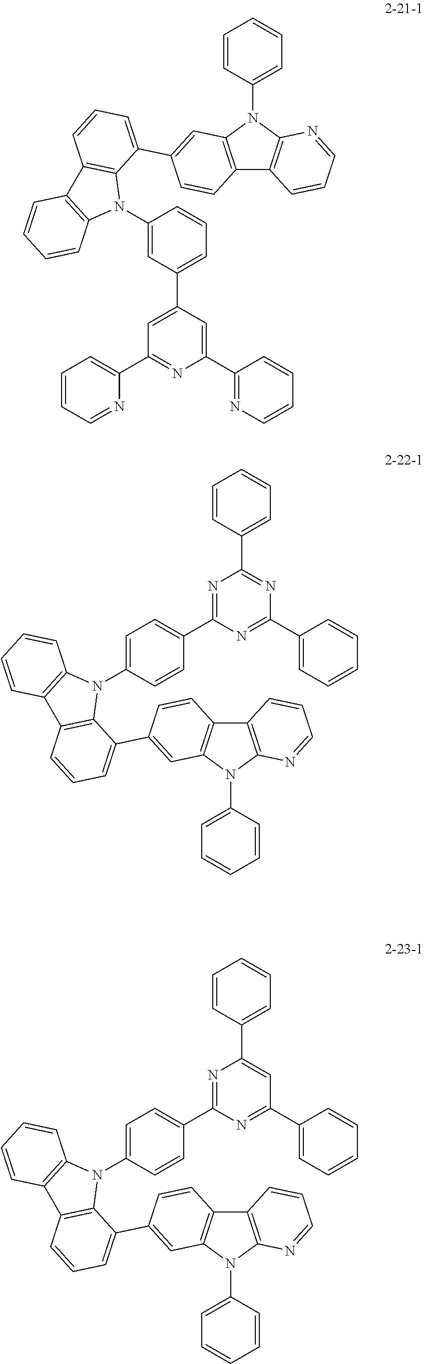 [SMILES: c1ccc(-n2c3cc(-c4cccc5c6ccccc6n(-c6cccc(-c7cc(-c8ccccn8)nc(-c8ccccn8)c7)c6)c45)ccc3c3cccnc32)cc1 ; c1ccc(-c2nc(-c3ccccc3)nc(-c3ccc(-n4c5ccccc5c5cccc(-c6ccc7c8cccnc8n(-c8ccccc8)c7c6)c54)cc3)n2)cc1 ; c1ccc(-c2cc(-c3ccccc3)nc(-c3ccc(-n4c5ccccc5c5cccc(-c6ccc7c8cccnc8n(-c8ccccc8)c7c6)c54)cc3)n2)cc1]

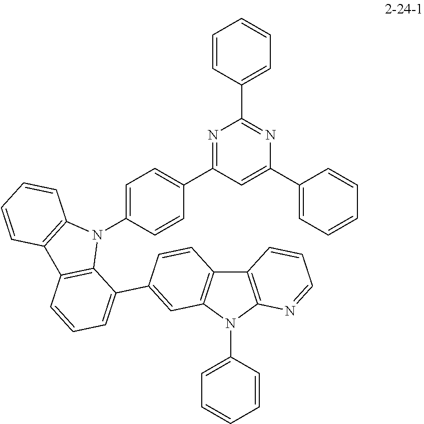 [SMILES: c1ccc(-c2cc(-c3ccc(-n4c5ccccc5c5cccc(-c6ccc7c8cccnc8n(-c8ccccc8)c7c6)c54)cc3)nc(-c3ccccc3)n2)cc1]

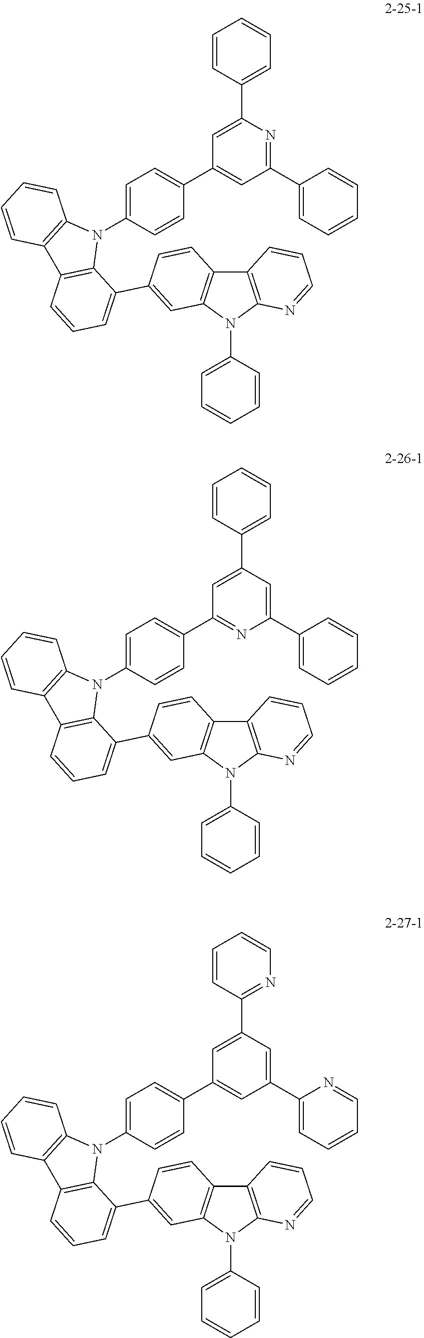

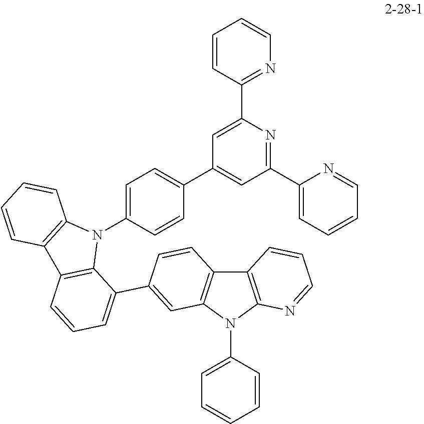 [SMILES: c1ccc(-n2c3cc(-c4cccc5c6ccccc6n(-c6ccc(-c7cc(-c8ccccn8)nc(-c8ccccn8)c7)cc6)c45)ccc3c3cccnc32)cc1]

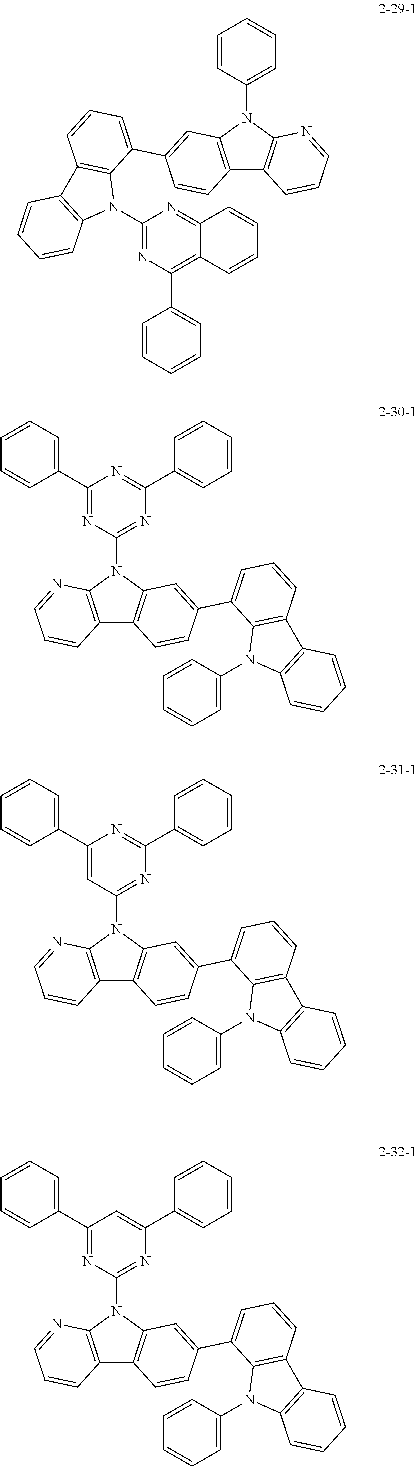 [SMILES: c1ccc(-c2nc(-n3c4ccccc4c4cccc(-c5ccc6c7cccnc7n(-c7ccccc7)c6c5)c43)nc3ccccc23)cc1 ; c1ccc(-c2nc(-c3ccccc3)nc(-n3c4cc(-c5cccc6c7ccccc7n(-c7ccccc7)c56)ccc4c4cccnc43)n2)cc1 ; c1ccc(-c2cc(-n3c4cc(-c5cccc6c7ccccc7n(-c7ccccc7)c56)ccc4c4cccnc43)nc(-c3ccccc3)n2)cc1 ; c1ccc(-c2cc(-c3ccccc3)nc(-n3c4cc(-c5cccc6c7ccccc7n(-c7ccccc7)c56)ccc4c4cccnc43)n2)cc1]

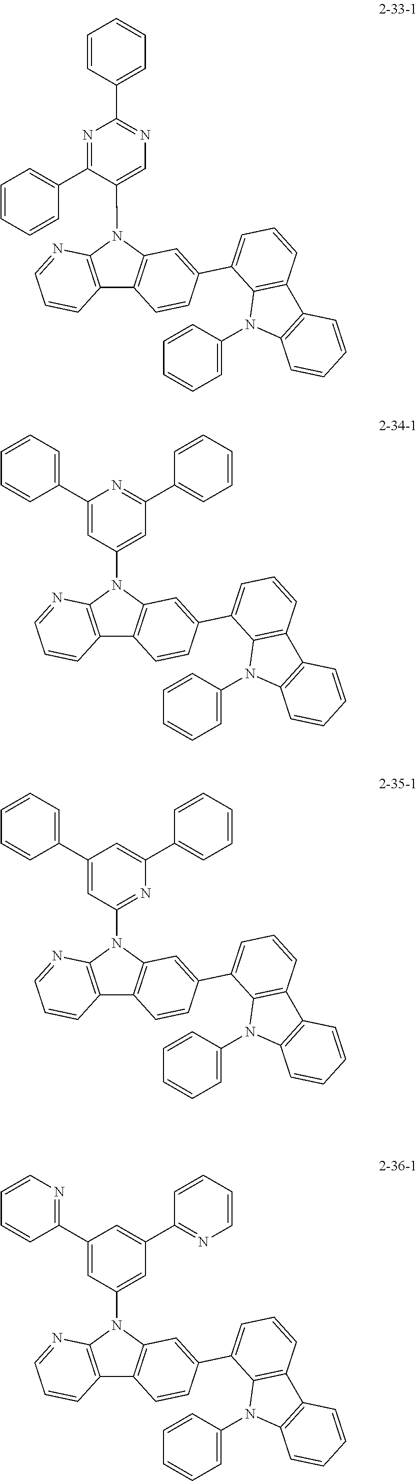

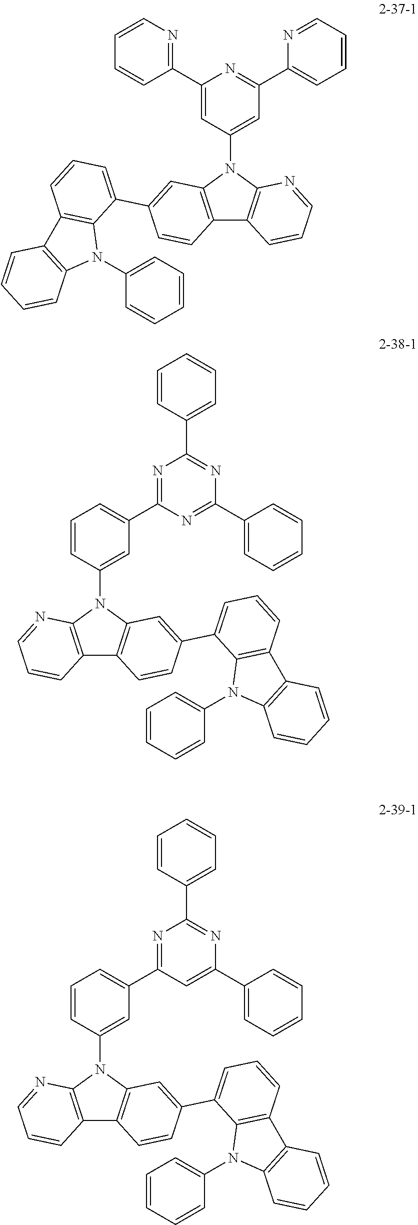

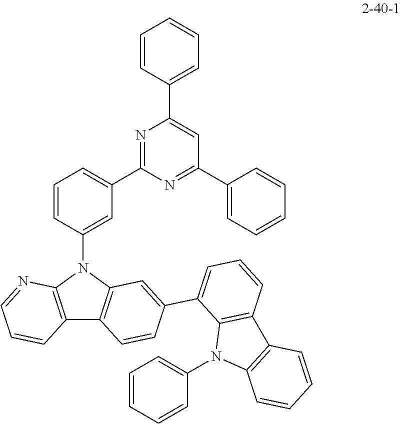 [SMILES: c1ccc(-c2cc(-c3ccccc3)nc(-c3cccc(-n4c5cc(-c6cccc7c8ccccc8n(-c8ccccc8)c67)ccc5c5cccnc54)c3)n2)cc1]

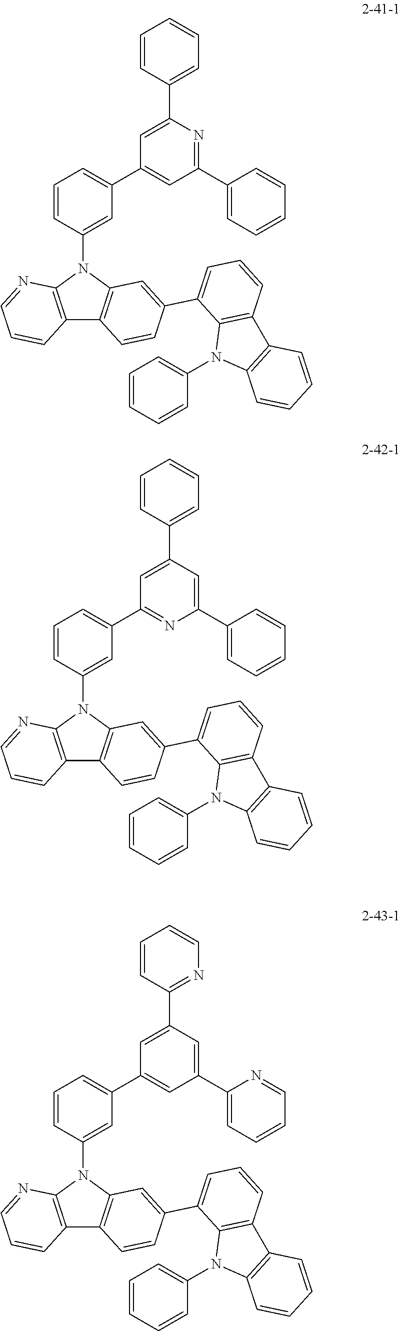

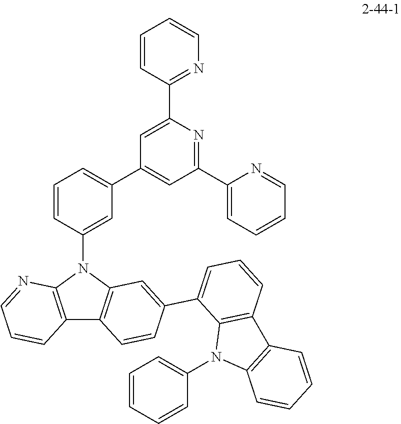 [SMILES: c1ccc(-n2c3ccccc3c3cccc(-c4ccc5c6cccnc6n(-c6cccc(-c7cc(-c8ccccn8)nc(-c8ccccn8)c7)c6)c5c4)c32)cc1]

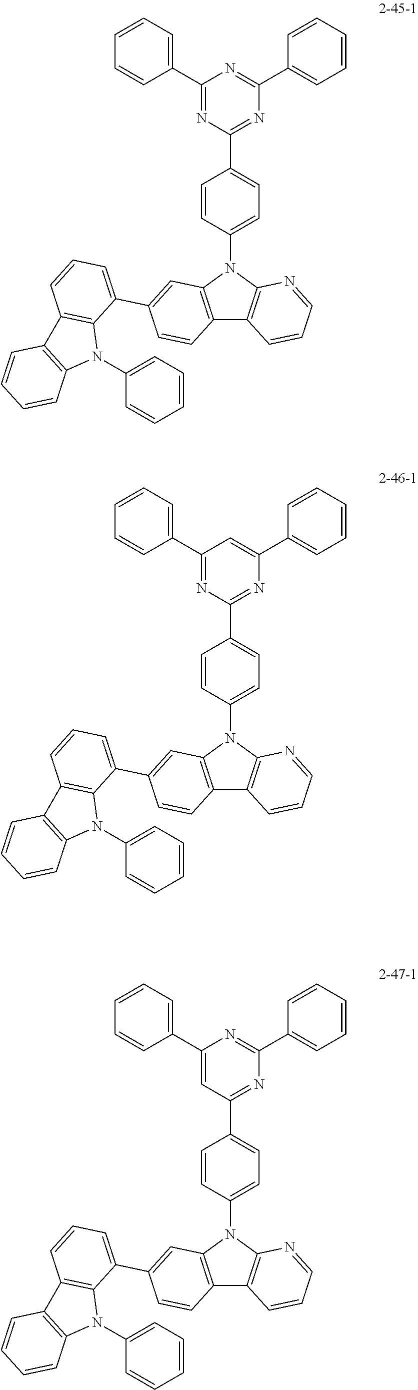 [SMILES: c1ccc(-c2nc(-c3ccccc3)nc(-c3ccc(-n4c5cc(-c6cccc7c8ccccc8n(-c8ccccc8)c67)ccc5c5cccnc54)cc3)n2)cc1 ; c1ccc(-c2cc(-c3ccccc3)nc(-c3ccc(-n4c5cc(-c6cccc7c8ccccc8n(-c8ccccc8)c67)ccc5c5cccnc54)cc3)n2)cc1 ; c1ccc(-c2cc(-c3ccc(-n4c5cc(-c6cccc7c8ccccc8n(-c8ccccc8)c67)ccc5c5cccnc54)cc3)nc(-c3ccccc3)n2)cc1]

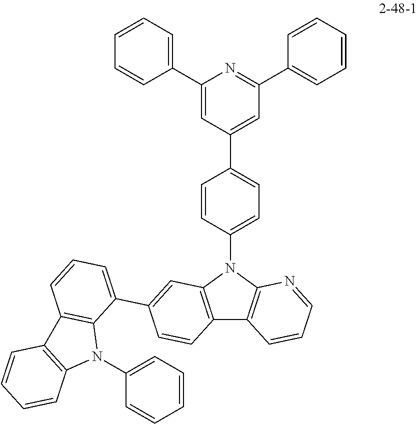 [SMILES: c1ccc(-c2cc(-c3ccc(-n4c5cc(-c6cccc7c8ccccc8n(-c8ccccc8)c67)ccc5c5cccnc54)cc3)cc(-c3ccccc3)n2)cc1]

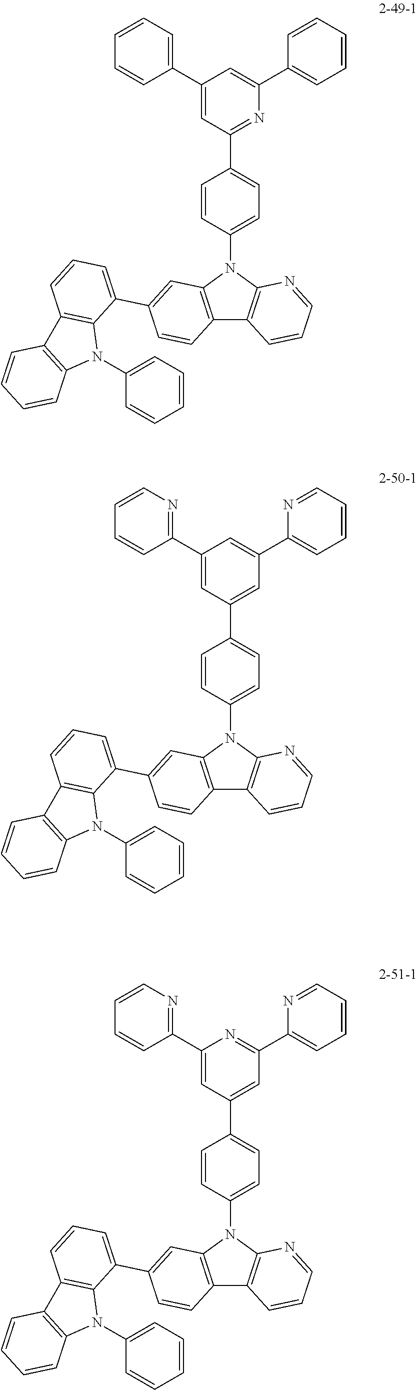 [SMILES: c1ccc(-c2cc(-c3ccccc3)nc(-c3ccc(-n4c5cc(-c6cccc7c8ccccc8n(-c8ccccc8)c67)ccc5c5cccnc54)cc3)c2)cc1 ; c1ccc(-n2c3ccccc3c3cccc(-c4ccc5c6cccnc6n(-c6ccc(-c7cc(-c8ccccn8)cc(-c8ccccn8)c7)cc6)c5c4)c32)cc1 ; c1ccc(-n2c3ccccc3c3cccc(-c4ccc5c6cccnc6n(-c6ccc(-c7cc(-c8ccccn8)nc(-c8ccccn8)c7)cc6)c5c4)c32)cc1]

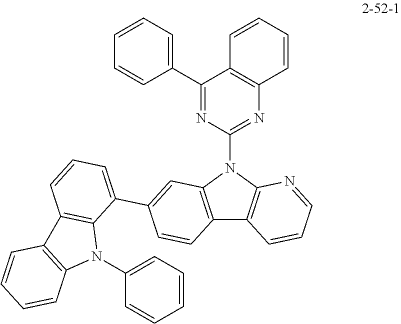 [SMILES: c1ccc(-c2nc(-n3c4cc(-c5cccc6c7ccccc7n(-c7ccccc7)c56)ccc4c4cccnc43)nc3ccccc23)cc1]

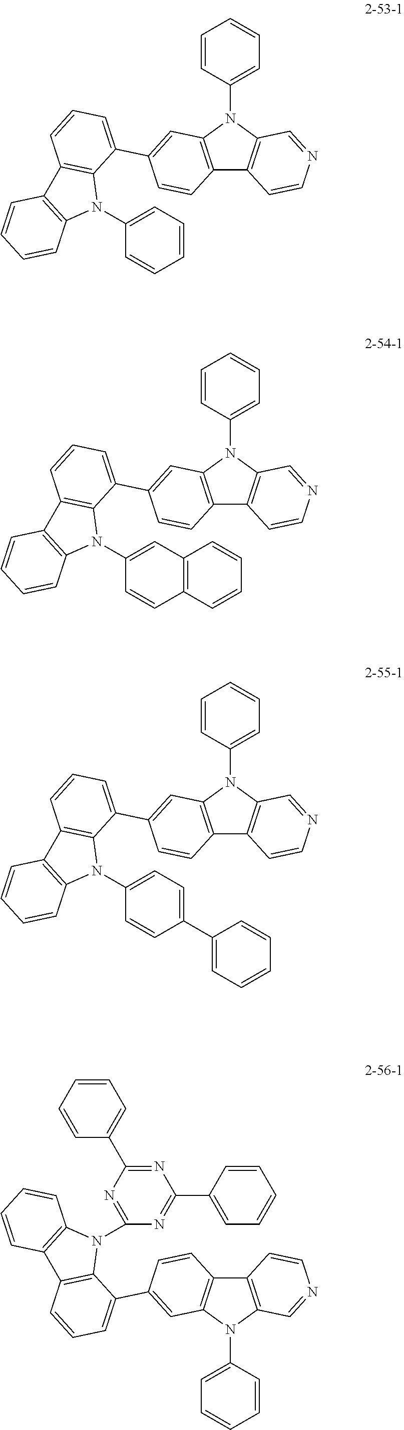 [SMILES: c1ccc(-n2c3cnccc3c3ccc(-c4cccc5c6ccccc6n(-c6ccccc6)c45)cc32)cc1 ; c1ccc(-n2c3cnccc3c3ccc(-c4cccc5c6ccccc6n(-c6ccc7ccccc7c6)c45)cc32)cc1 ; c1ccc(-c2ccc(-n3c4ccccc4c4cccc(-c5ccc6c7ccncc7n(-c7ccccc7)c6c5)c43)cc2)cc1 ; c1ccc(-c2nc(-c3ccccc3)nc(-n3c4ccccc4c4cccc(-c5ccc6c7ccncc7n(-c7ccccc7)c6c5)c43)n2)cc1]

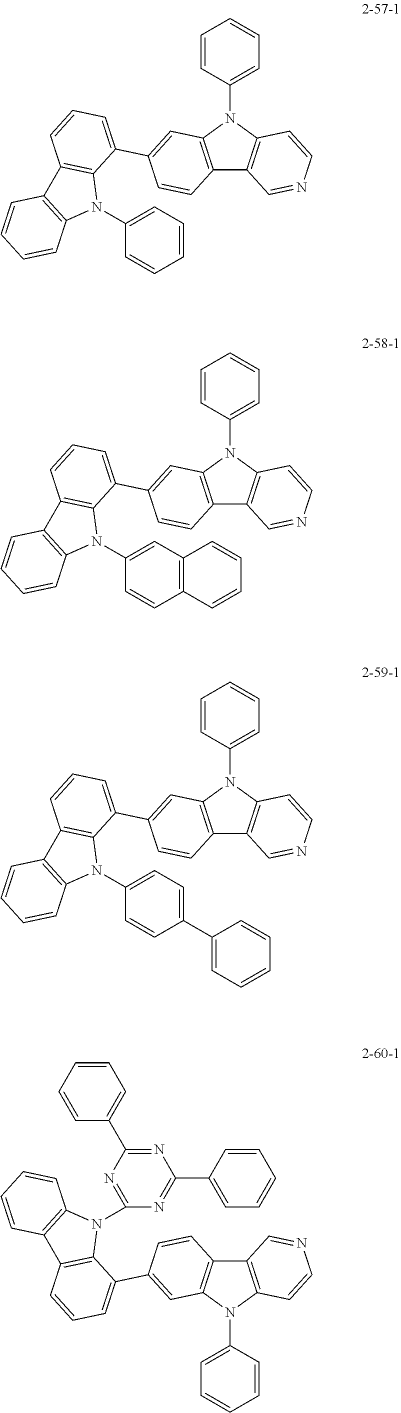 [SMILES: c1ccc(-n2c3ccncc3c3ccc(-c4cccc5c6ccccc6n(-c6ccccc6)c45)cc32)cc1 ; c1ccc(-n2c3ccncc3c3ccc(-c4cccc5c6ccccc6n(-c6ccc7ccccc7c6)c45)cc32)cc1 ; c1ccc(-c2ccc(-n3c4ccccc4c4cccc(-c5ccc6c7cnccc7n(-c7ccccc7)c6c5)c43)cc2)cc1 ; c1ccc(-c2nc(-c3ccccc3)nc(-n3c4ccccc4c4cccc(-c5ccc6c7cnccc7n(-c7ccccc7)c6c5)c43)n2)cc1]

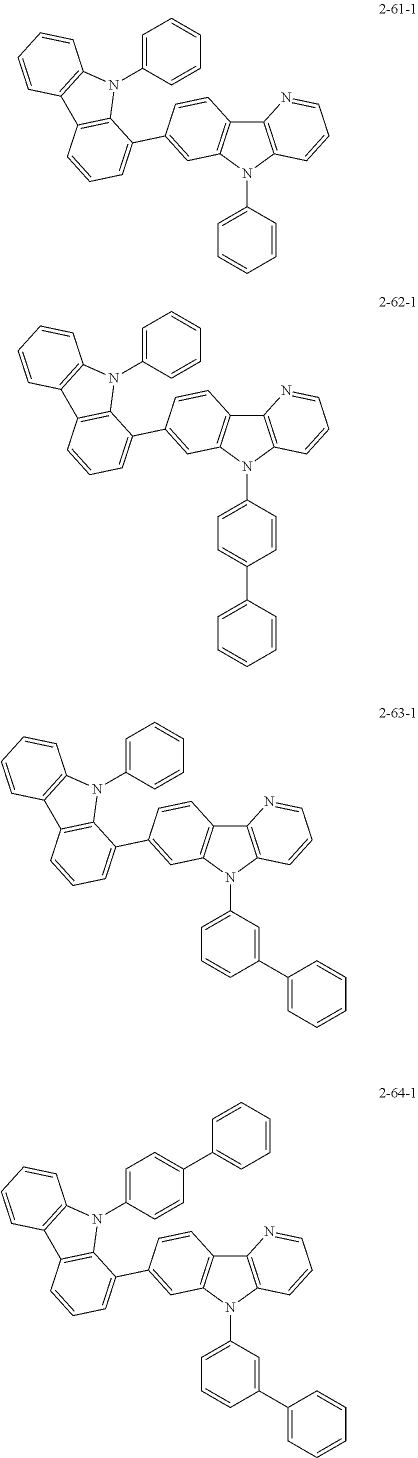 [SMILES: c1ccc(-n2c3cc(-c4cccc5c6ccccc6n(-c6ccccc6)c45)ccc3c3ncccc32)cc1 ; c1ccc(-c2ccc(-n3c4cc(-c5cccc6c7ccccc7n(-c7ccccc7)c56)ccc4c4ncccc43)cc2)cc1 ; c1ccc(-c2cccc(-n3c4cc(-c5cccc6c7ccccc7n(-c7ccccc7)c56)ccc4c4ncccc43)c2)cc1 ; c1ccc(-c2ccc(-n3c4ccccc4c4cccc(-c5ccc6c7ncccc7n(-c7cccc(-c8ccccc8)c7)c6c5)c43)cc2)cc1]

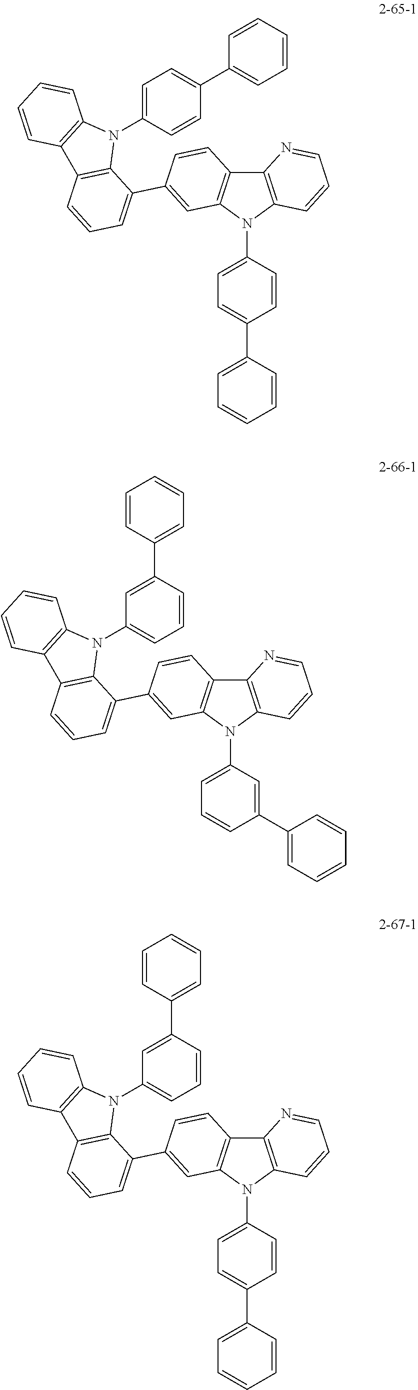 [SMILES: c1ccc(-c2ccc(-n3c4cc(-c5cccc6c7ccccc7n(-c7ccc(-c8ccccc8)cc7)c56)ccc4c4ncccc43)cc2)cc1 ; c1ccc(-c2cccc(-n3c4cc(-c5cccc6c7ccccc7n(-c7cccc(-c8ccccc8)c7)c56)ccc4c4ncccc43)c2)cc1 ; c1ccc(-c2ccc(-n3c4cc(-c5cccc6c7ccccc7n(-c7cccc(-c8ccccc8)c7)c56)ccc4c4ncccc43)cc2)cc1]

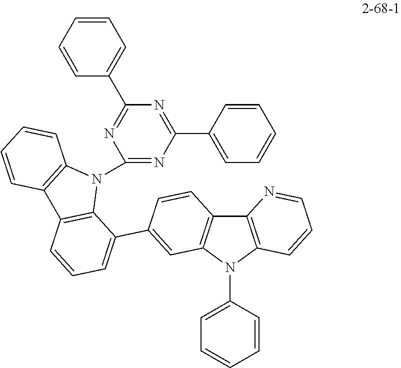 [SMILES: c1ccc(-c2nc(-c3ccccc3)nc(-n3c4ccccc4c4cccc(-c5ccc6c7ncccc7n(-c7ccccc7)c6c5)c43)n2)cc1]

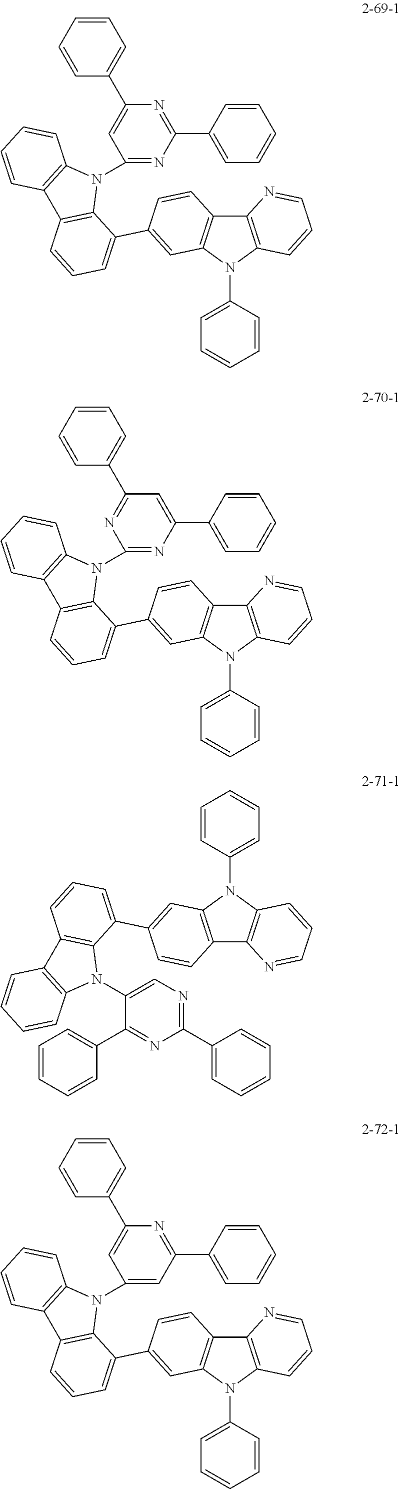

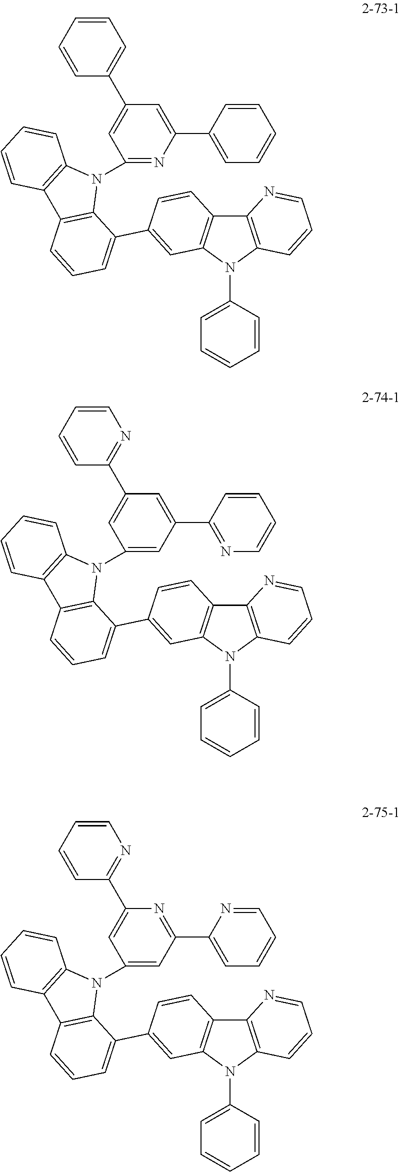 [SMILES: c1ccc(-c2cc(-c3ccccc3)nc(-n3c4ccccc4c4cccc(-c5ccc6c7ncccc7n(-c7ccccc7)c6c5)c43)c2)cc1 ; c1ccc(-n2c3cc(-c4cccc5c6ccccc6n(-c6cc(-c7ccccn7)cc(-c7ccccn7)c6)c45)ccc3c3ncccc32)cc1 ; c1ccc(-n2c3cc(-c4cccc5c6ccccc6n(-c6cc(-c7ccccn7)nc(-c7ccccn7)c6)c45)ccc3c3ncccc32)cc1]

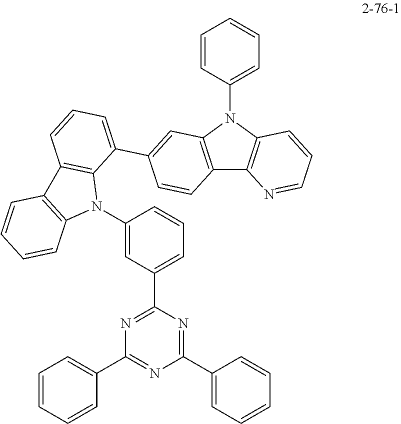 [SMILES: c1ccc(-c2nc(-c3ccccc3)nc(-c3cccc(-n4c5ccccc5c5cccc(-c6ccc7c8ncccc8n(-c8ccccc8)c7c6)c54)c3)n2)cc1]

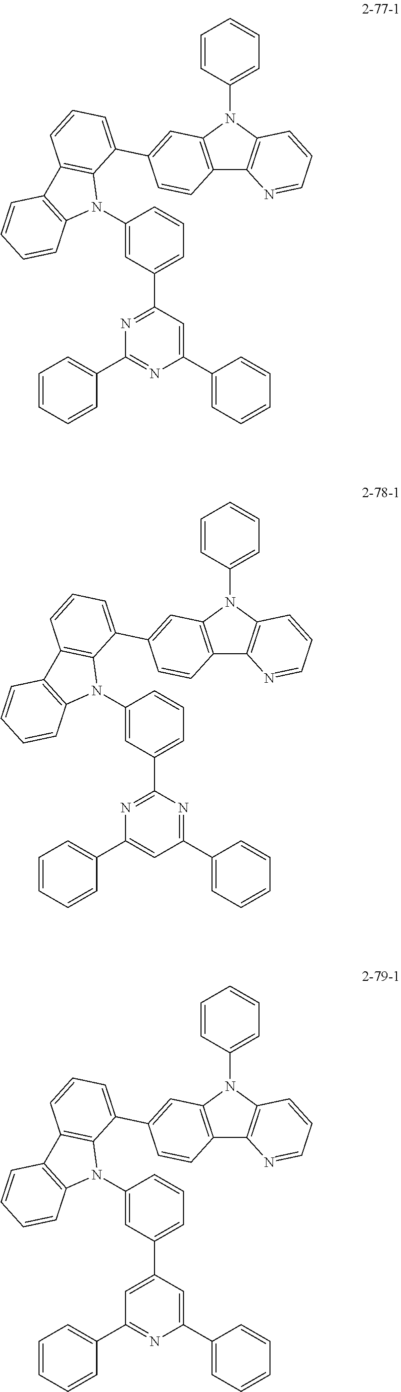 [SMILES: c1ccc(-c2cc(-c3cccc(-n4c5ccccc5c5cccc(-c6ccc7c8ncccc8n(-c8ccccc8)c7c6)c54)c3)nc(-c3ccccc3)n2)cc1 ; c1ccc(-c2cc(-c3ccccc3)nc(-c3cccc(-n4c5ccccc5c5cccc(-c6ccc7c8ncccc8n(-c8ccccc8)c7c6)c54)c3)n2)cc1 ; c1ccc(-c2cc(-c3cccc(-n4c5ccccc5c5cccc(-c6ccc7c8ncccc8n(-c8ccccc8)c7c6)c54)c3)cc(-c3ccccc3)n2)cc1]

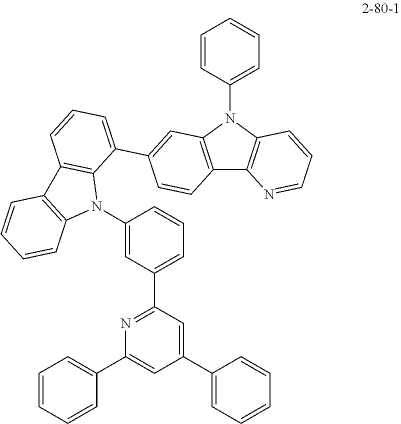 [SMILES: c1ccc(-c2cc(-c3ccccc3)nc(-c3cccc(-n4c5ccccc5c5cccc(-c6ccc7c8ncccc8n(-c8ccccc8)c7c6)c54)c3)c2)cc1]

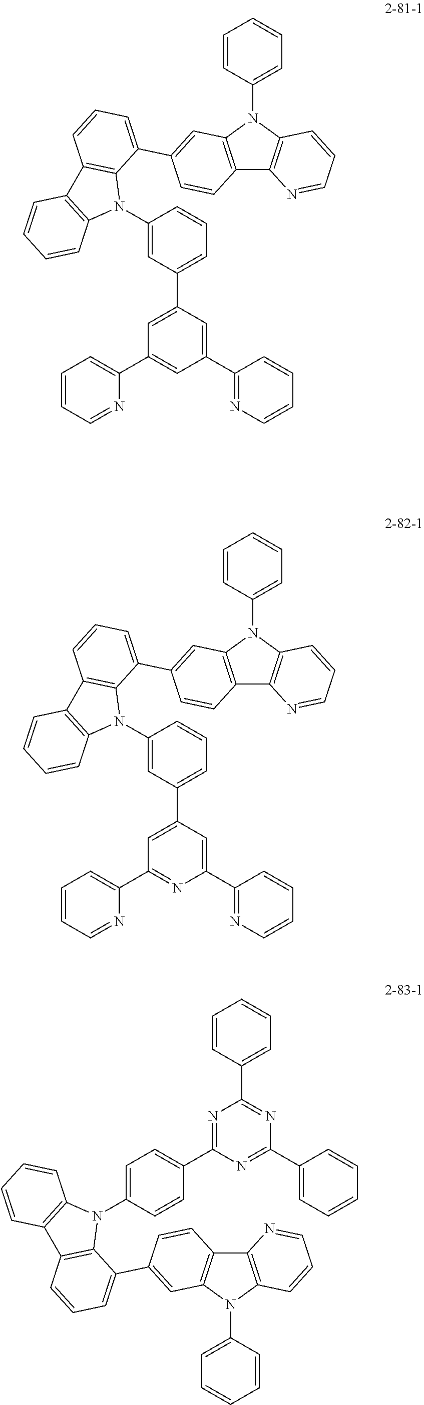 [SMILES: c1ccc(-n2c3cc(-c4cccc5c6ccccc6n(-c6cccc(-c7cc(-c8ccccn8)cc(-c8ccccn8)c7)c6)c45)ccc3c3ncccc32)cc1 ; c1ccc(-n2c3cc(-c4cccc5c6ccccc6n(-c6cccc(-c7cc(-c8ccccn8)nc(-c8ccccn8)c7)c6)c45)ccc3c3ncccc32)cc1 ; c1ccc(-c2nc(-c3ccccc3)nc(-c3ccc(-n4c5ccccc5c5cccc(-c6ccc7c8ncccc8n(-c8ccccc8)c7c6)c54)cc3)n2)cc1]

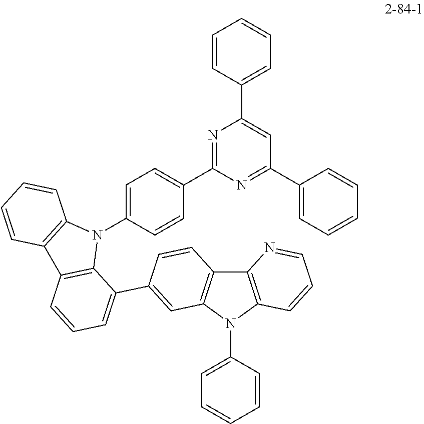 [SMILES: c1ccc(-c2cc(-c3ccccc3)nc(-c3ccc(-n4c5ccccc5c5cccc(-c6ccc7c8ncccc8n(-c8ccccc8)c7c6)c54)cc3)n2)cc1]

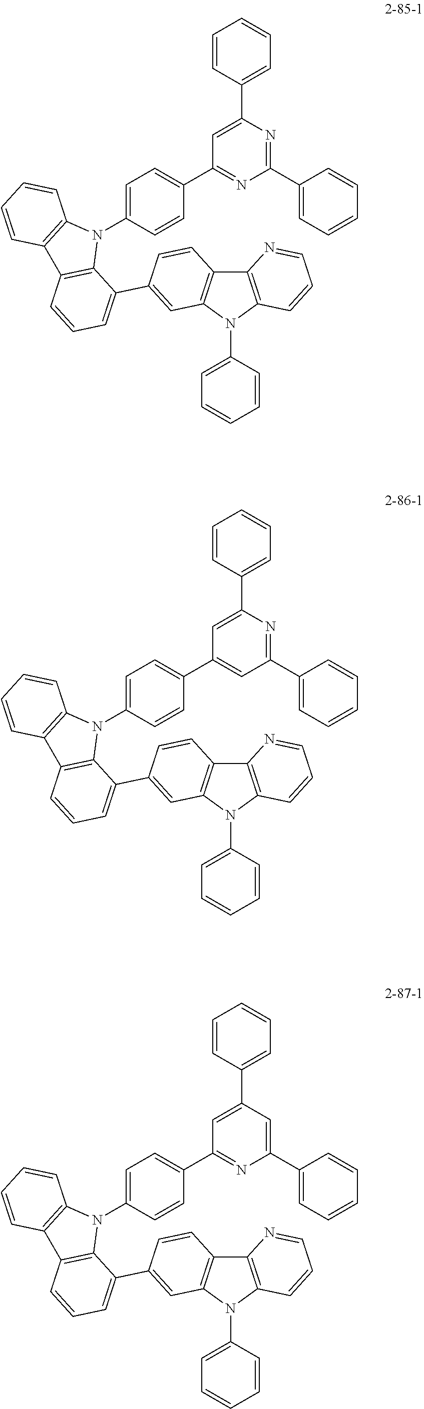 [SMILES: c1ccc(-c2cc(-c3ccc(-n4c5ccccc5c5cccc(-c6ccc7c8ncccc8n(-c8ccccc8)c7c6)c54)cc3)nc(-c3ccccc3)n2)cc1 ; c1ccc(-c2cc(-c3ccc(-n4c5ccccc5c5cccc(-c6ccc7c8ncccc8n(-c8ccccc8)c7c6)c54)cc3)cc(-c3ccccc3)n2)cc1 ; c1ccc(-c2cc(-c3ccccc3)nc(-c3ccc(-n4c5ccccc5c5cccc(-c6ccc7c8ncccc8n(-c8ccccc8)c7c6)c54)cc3)c2)cc1]

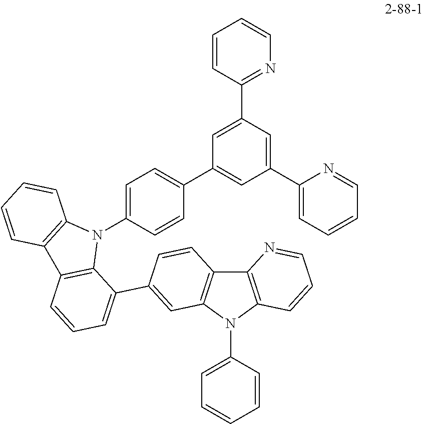 [SMILES: c1ccc(-n2c3cc(-c4cccc5c6ccccc6n(-c6ccc(-c7cc(-c8ccccn8)cc(-c8ccccn8)c7)cc6)c45)ccc3c3ncccc32)cc1]

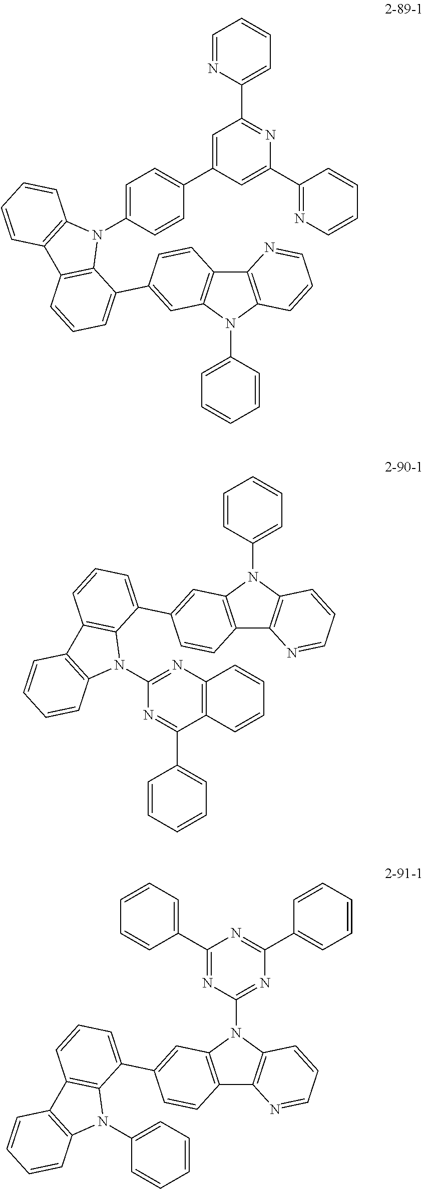 [SMILES: c1ccc(-n2c3cc(-c4cccc5c6ccccc6n(-c6ccc(-c7cc(-c8ccccn8)nc(-c8ccccn8)c7)cc6)c45)ccc3c3ncccc32)cc1 ; c1ccc(-c2nc(-n3c4ccccc4c4cccc(-c5ccc6c7ncccc7n(-c7ccccc7)c6c5)c43)nc3ccccc23)cc1 ; c1ccc(-c2nc(-c3ccccc3)nc(-n3c4cc(-c5cccc6c7ccccc7n(-c7ccccc7)c56)ccc4c4ncccc43)n2)cc1]

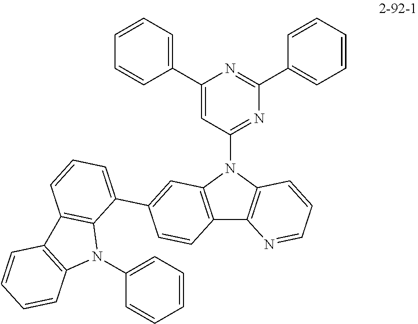 [SMILES: c1ccc(-c2cc(-n3c4cc(-c5cccc6c7ccccc7n(-c7ccccc7)c56)ccc4c4ncccc43)nc(-c3ccccc3)n2)cc1]

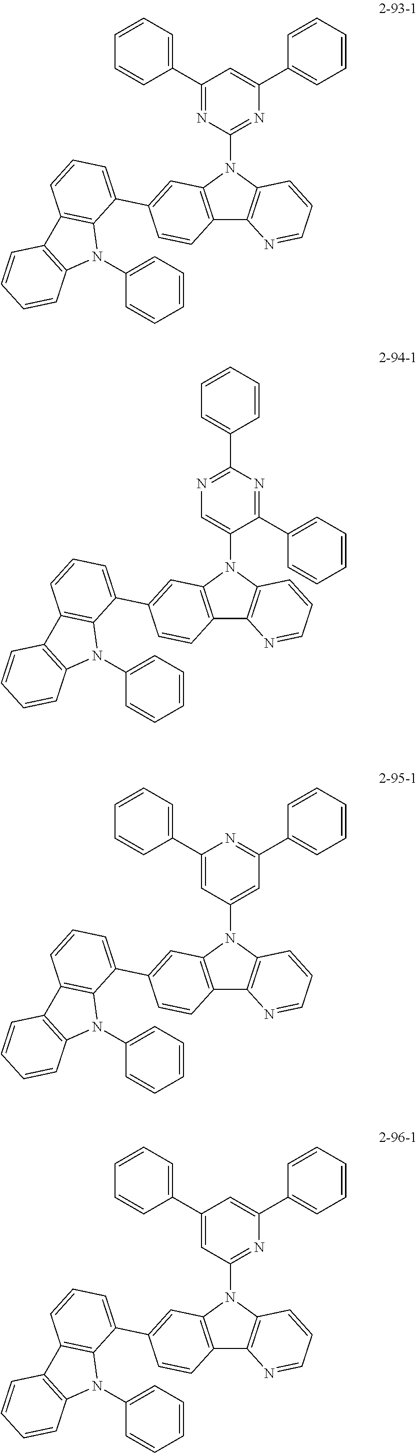 [SMILES: c1ccc(-c2cc(-c3ccccc3)nc(-n3c4cc(-c5cccc6c7ccccc7n(-c7ccccc7)c56)ccc4c4ncccc43)n2)cc1 ; c1ccc(-c2ncc(-n3c4cc(-c5cccc6c7ccccc7n(-c7ccccc7)c56)ccc4c4ncccc43)c(-c3ccccc3)n2)cc1 ; c1ccc(-c2cc(-n3c4cc(-c5cccc6c7ccccc7n(-c7ccccc7)c56)ccc4c4ncccc43)cc(-c3ccccc3)n2)cc1 ; c1ccc(-c2cc(-c3ccccc3)nc(-n3c4cc(-c5cccc6c7ccccc7n(-c7ccccc7)c56)ccc4c4ncccc43)c2)cc1]

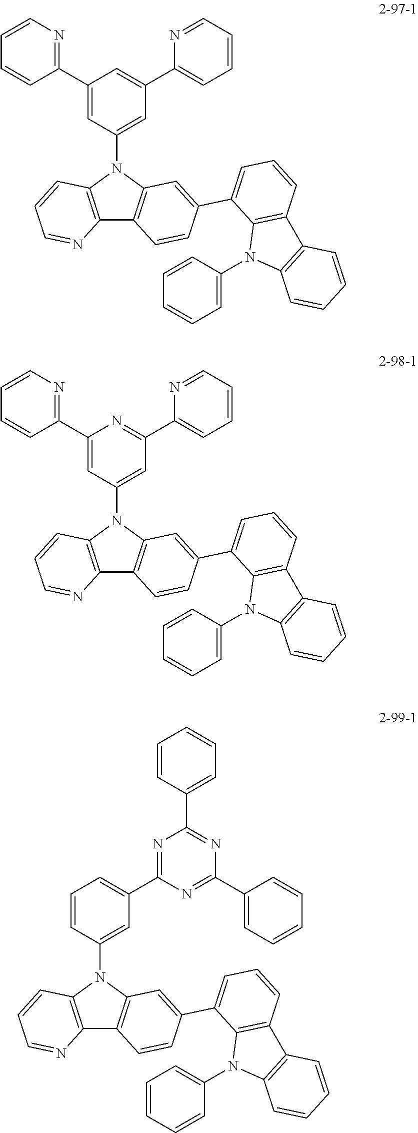 [SMILES: c1ccc(-n2c3ccccc3c3cccc(-c4ccc5c6ncccc6n(-c6cc(-c7ccccn7)cc(-c7ccccn7)c6)c5c4)c32)cc1 ; c1ccc(-n2c3ccccc3c3cccc(-c4ccc5c6ncccc6n(-c6cc(-c7ccccn7)nc(-c7ccccn7)c6)c5c4)c32)cc1 ; c1ccc(-c2nc(-c3ccccc3)nc(-c3cccc(-n4c5cc(-c6cccc7c8ccccc8n(-c8ccccc8)c67)ccc5c5ncccc54)c3)n2)cc1]

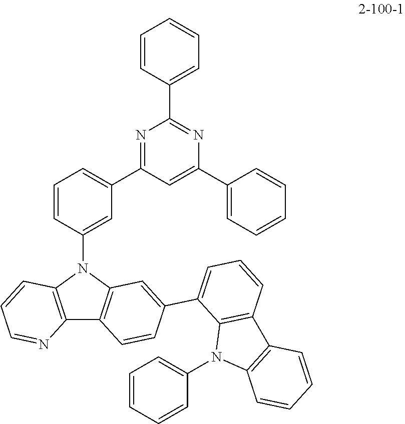 [SMILES: c1ccc(-c2cc(-c3cccc(-n4c5cc(-c6cccc7c8ccccc8n(-c8ccccc8)c67)ccc5c5ncccc54)c3)nc(-c3ccccc3)n2)cc1]

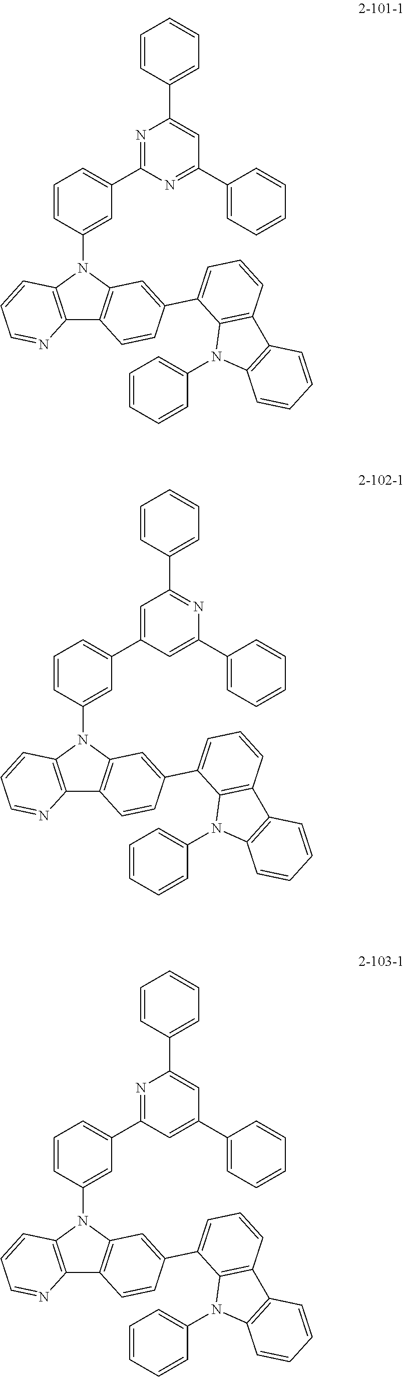 [SMILES: c1ccc(-c2cc(-c3ccccc3)nc(-c3cccc(-n4c5cc(-c6cccc7c8ccccc8n(-c8ccccc8)c67)ccc5c5ncccc54)c3)n2)cc1 ; c1ccc(-c2cc(-c3cccc(-n4c5cc(-c6cccc7c8ccccc8n(-c8ccccc8)c67)ccc5c5ncccc54)c3)cc(-c3ccccc3)n2)cc1 ; c1ccc(-c2cc(-c3ccccc3)nc(-c3cccc(-n4c5cc(-c6cccc7c8ccccc8n(-c8ccccc8)c67)ccc5c5ncccc54)c3)c2)cc1]

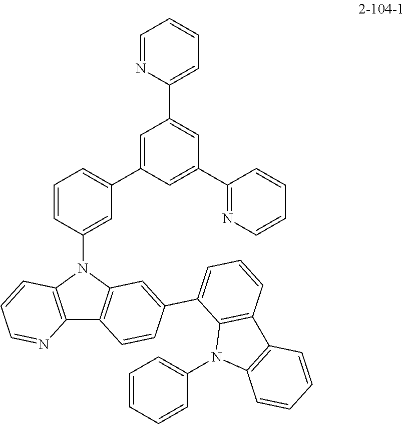 [SMILES: c1ccc(-n2c3ccccc3c3cccc(-c4ccc5c6ncccc6n(-c6cccc(-c7cc(-c8ccccn8)cc(-c8ccccn8)c7)c6)c5c4)c32)cc1]

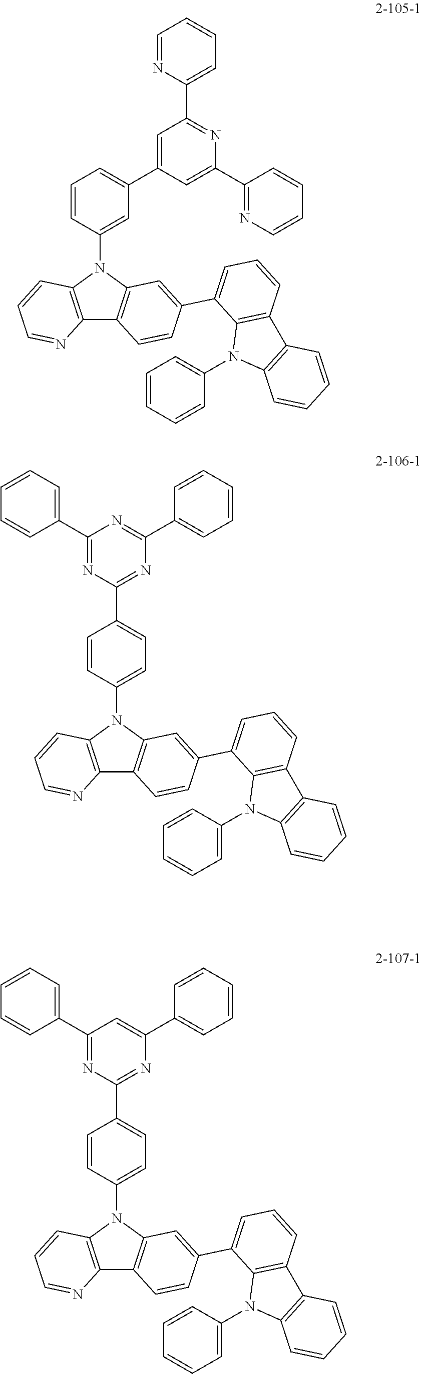 [SMILES: c1ccc(-n2c3ccccc3c3cccc(-c4ccc5c6ncccc6n(-c6cccc(-c7cc(-c8ccccn8)nc(-c8ccccn8)c7)c6)c5c4)c32)cc1 ; c1ccc(-c2nc(-c3ccccc3)nc(-c3ccc(-n4c5cc(-c6cccc7c8ccccc8n(-c8ccccc8)c67)ccc5c5ncccc54)cc3)n2)cc1 ; c1ccc(-c2cc(-c3ccccc3)nc(-c3ccc(-n4c5cc(-c6cccc7c8ccccc8n(-c8ccccc8)c67)ccc5c5ncccc54)cc3)n2)cc1]

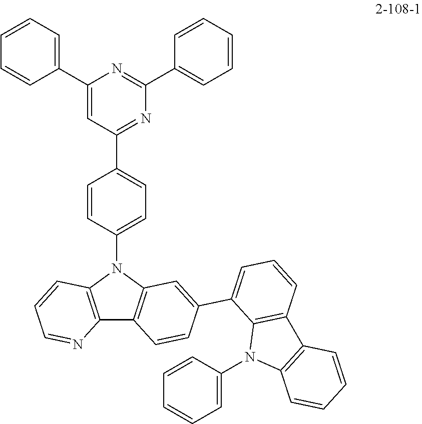 [SMILES: c1ccc(-c2cc(-c3ccc(-n4c5cc(-c6cccc7c8ccccc8n(-c8ccccc8)c67)ccc5c5ncccc54)cc3)nc(-c3ccccc3)n2)cc1]

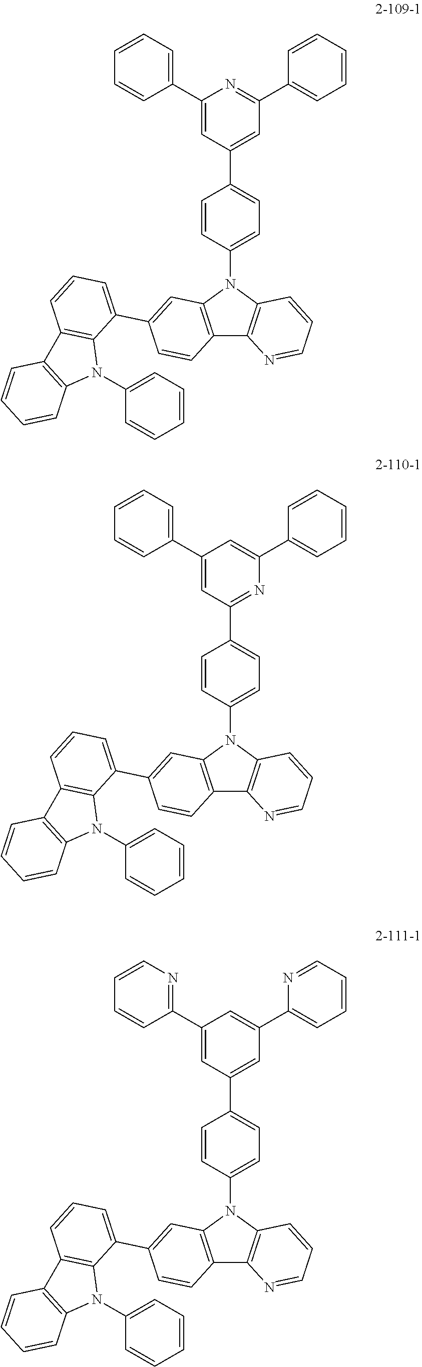 [SMILES: c1ccc(-c2cc(-c3ccc(-n4c5cc(-c6cccc7c8ccccc8n(-c8ccccc8)c67)ccc5c5ncccc54)cc3)cc(-c3ccccc3)n2)cc1 ; c1ccc(-c2cc(-c3ccccc3)nc(-c3ccc(-n4c5cc(-c6cccc7c8ccccc8n(-c8ccccc8)c67)ccc5c5ncccc54)cc3)c2)cc1 ; c1ccc(-n2c3ccccc3c3cccc(-c4ccc5c6ncccc6n(-c6ccc(-c7cc(-c8ccccn8)cc(-c8ccccn8)c7)cc6)c5c4)c32)cc1]

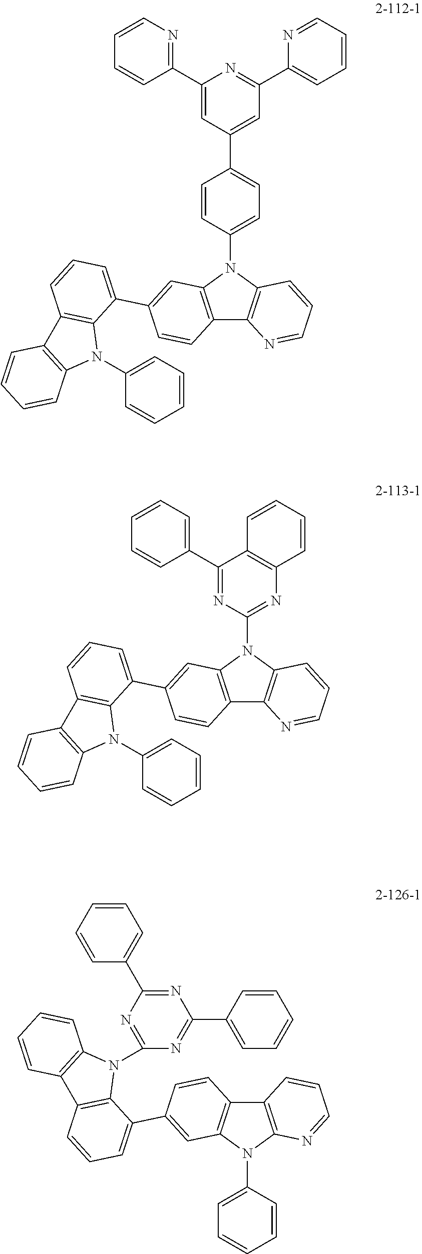 [SMILES: c1ccc(-n2c3ccccc3c3cccc(-c4ccc5c6ncccc6n(-c6ccc(-c7cc(-c8ccccn8)nc(-c8ccccn8)c7)cc6)c5c4)c32)cc1 ; c1ccc(-c2nc(-n3c4cc(-c5cccc6c7ccccc7n(-c7ccccc7)c56)ccc4c4ncccc43)nc3ccccc23)cc1 ; c1ccc(-c2nc(-c3ccccc3)nc(-n3c4ccccc4c4cccc(-c5ccc6c7cccnc7n(-c7ccccc7)c6c5)c43)n2)cc1]

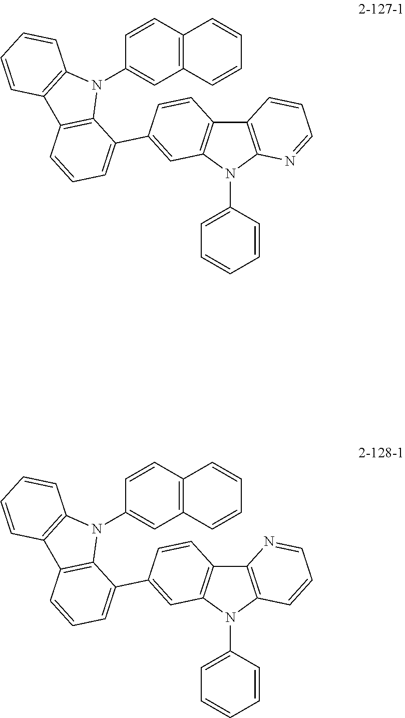 [SMILES: c1ccc(-n2c3cc(-c4cccc5c6ccccc6n(-c6ccc7ccccc7c6)c45)ccc3c3cccnc32)cc1 ; c1ccc(-n2c3cc(-c4cccc5c6ccccc6n(-c6ccc7ccccc7c6)c45)ccc3c3ncccc32)cc1]

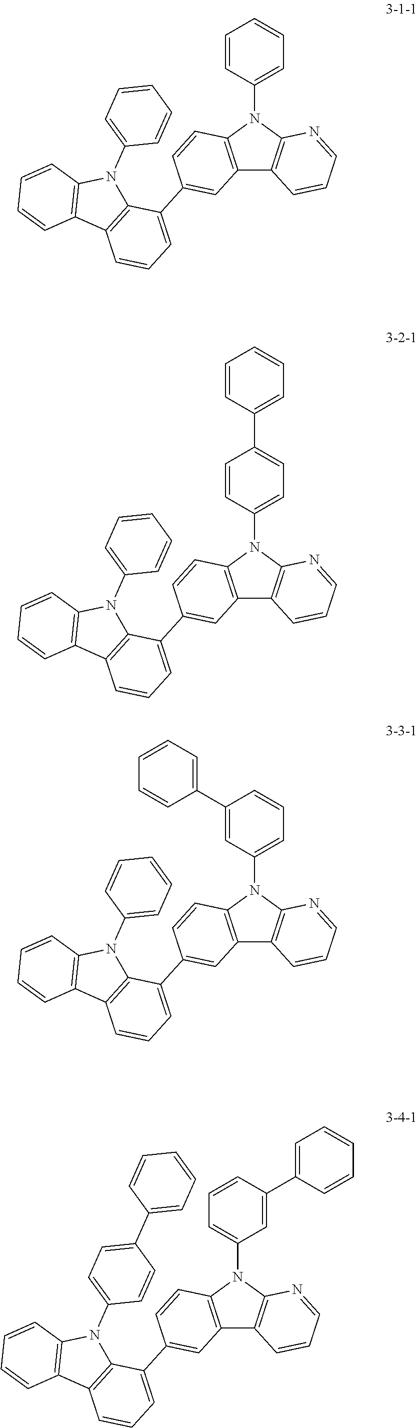 [SMILES: c1ccc(-n2c3ccc(-c4cccc5c6ccccc6n(-c6ccccc6)c45)cc3c3cccnc32)cc1 ; c1ccc(-c2ccc(-n3c4ccc(-c5cccc6c7ccccc7n(-c7ccccc7)c56)cc4c4cccnc43)cc2)cc1 ; c1ccc(-c2cccc(-n3c4ccc(-c5cccc6c7ccccc7n(-c7ccccc7)c56)cc4c4cccnc43)c2)cc1 ; c1ccc(-c2ccc(-n3c4ccccc4c4cccc(-c5ccc6c(c5)c5cccnc5n6-c5cccc(-c6ccccc6)c5)c43)cc2)cc1]

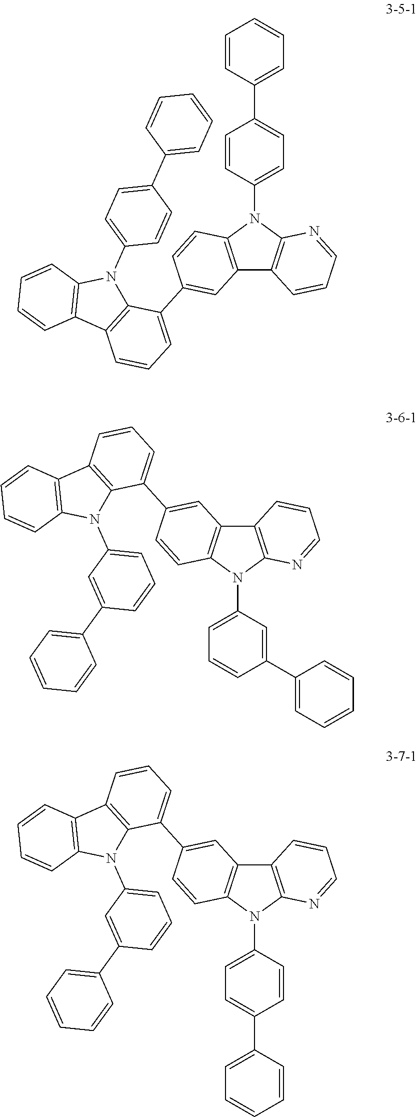 [SMILES: c1ccc(-c2ccc(-n3c4ccc(-c5cccc6c7ccccc7n(-c7ccc(-c8ccccc8)cc7)c56)cc4c4cccnc43)cc2)cc1 ; c1ccc(-c2cccc(-n3c4ccc(-c5cccc6c7ccccc7n(-c7cccc(-c8ccccc8)c7)c56)cc4c4cccnc43)c2)cc1 ; c1ccc(-c2ccc(-n3c4ccc(-c5cccc6c7ccccc7n(-c7cccc(-c8ccccc8)c7)c56)cc4c4cccnc43)cc2)cc1]

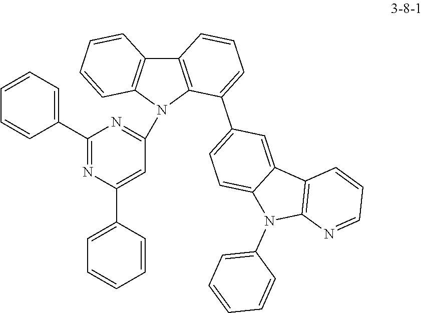 [SMILES: c1ccc(-c2cc(-n3c4ccccc4c4cccc(-c5ccc6c(c5)c5cccnc5n6-c5ccccc5)c43)nc(-c3ccccc3)n2)cc1]

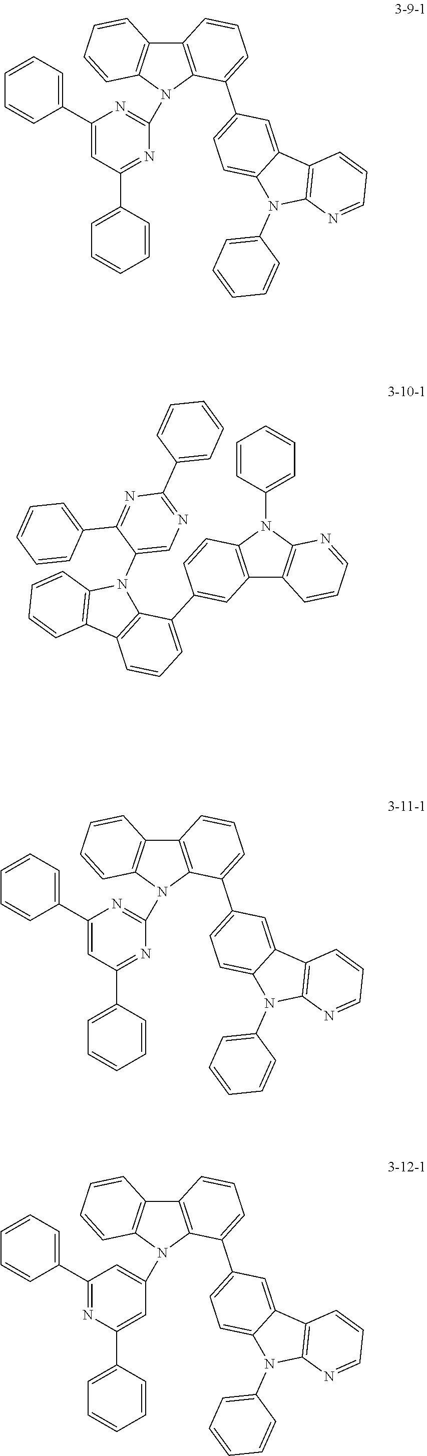 [SMILES: c1ccc(-c2cc(-c3ccccc3)nc(-n3c4ccccc4c4cccc(-c5ccc6c(c5)c5cccnc5n6-c5ccccc5)c43)n2)cc1 ; c1ccc(-c2ncc(-n3c4ccccc4c4cccc(-c5ccc6c(c5)c5cccnc5n6-c5ccccc5)c43)c(-c3ccccc3)n2)cc1 ; c1ccc(-c2cc(-c3ccccc3)nc(-n3c4ccccc4c4cccc(-c5ccc6c(c5)c5cccnc5n6-c5ccccc5)c43)n2)cc1 ; c1ccc(-c2cc(-n3c4ccccc4c4cccc(-c5ccc6c(c5)c5cccnc5n6-c5ccccc5)c43)cc(-c3ccccc3)n2)cc1]

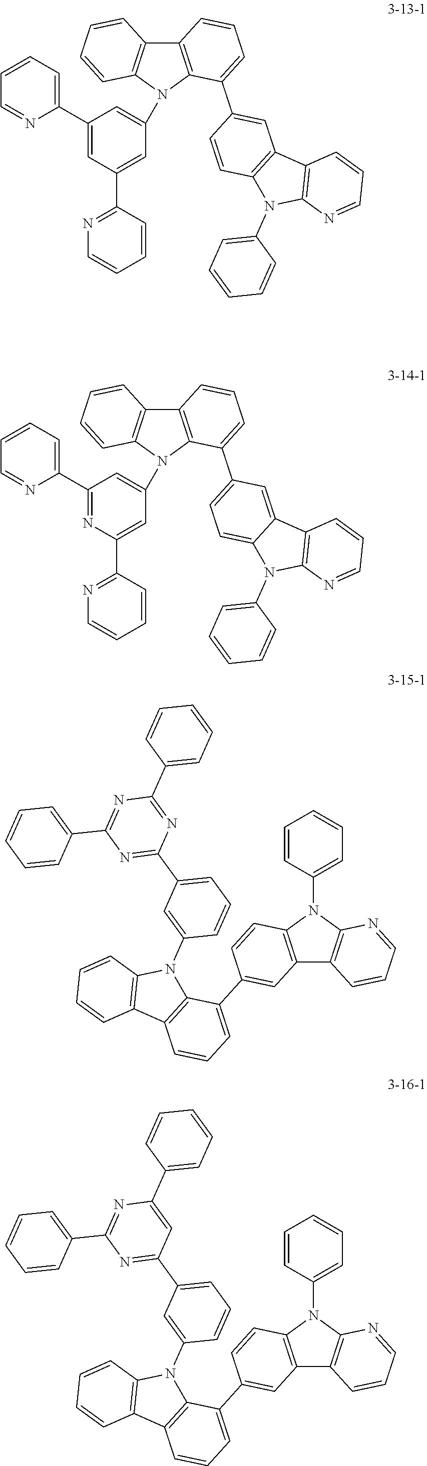 [SMILES: c1ccc(-n2c3ccc(-c4cccc5c6ccccc6n(-c6cc(-c7ccccn7)cc(-c7ccccn7)c6)c45)cc3c3cccnc32)cc1 ; c1ccc(-n2c3ccc(-c4cccc5c6ccccc6n(-c6cc(-c7ccccn7)nc(-c7ccccn7)c6)c45)cc3c3cccnc32)cc1 ; c1ccc(-c2nc(-c3ccccc3)nc(-c3cccc(-n4c5ccccc5c5cccc(-c6ccc7c(c6)c6cccnc6n7-c6ccccc6)c54)c3)n2)cc1 ; c1ccc(-c2cc(-c3cccc(-n4c5ccccc5c5cccc(-c6ccc7c(c6)c6cccnc6n7-c6ccccc6)c54)c3)nc(-c3ccccc3)n2)cc1]

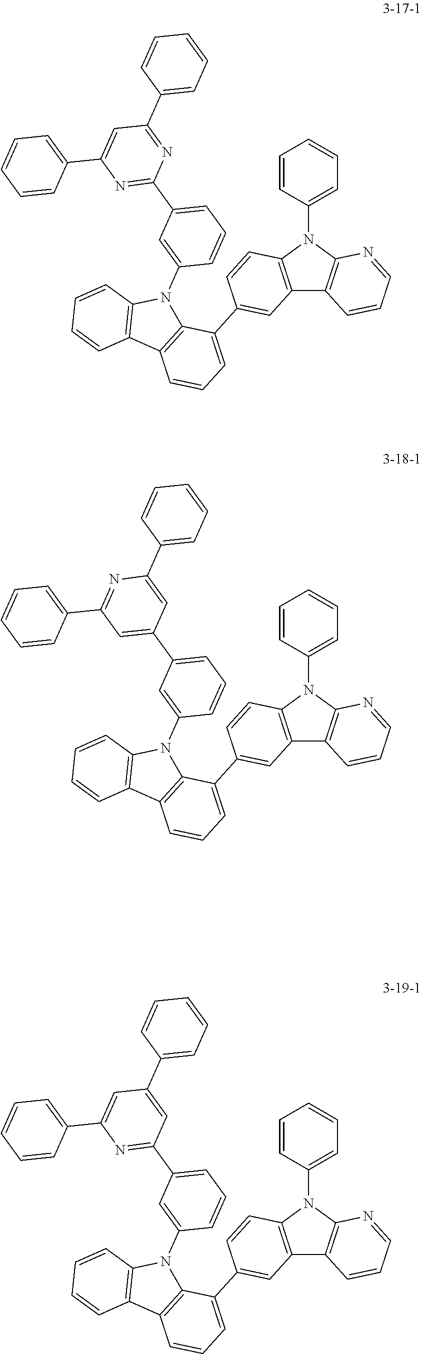 [SMILES: c1ccc(-c2cc(-c3ccccc3)nc(-c3cccc(-n4c5ccccc5c5cccc(-c6ccc7c(c6)c6cccnc6n7-c6ccccc6)c54)c3)n2)cc1 ; c1ccc(-c2cc(-c3cccc(-n4c5ccccc5c5cccc(-c6ccc7c(c6)c6cccnc6n7-c6ccccc6)c54)c3)cc(-c3ccccc3)n2)cc1 ; c1ccc(-c2cc(-c3ccccc3)nc(-c3cccc(-n4c5ccccc5c5cccc(-c6ccc7c(c6)c6cccnc6n7-c6ccccc6)c54)c3)c2)cc1]

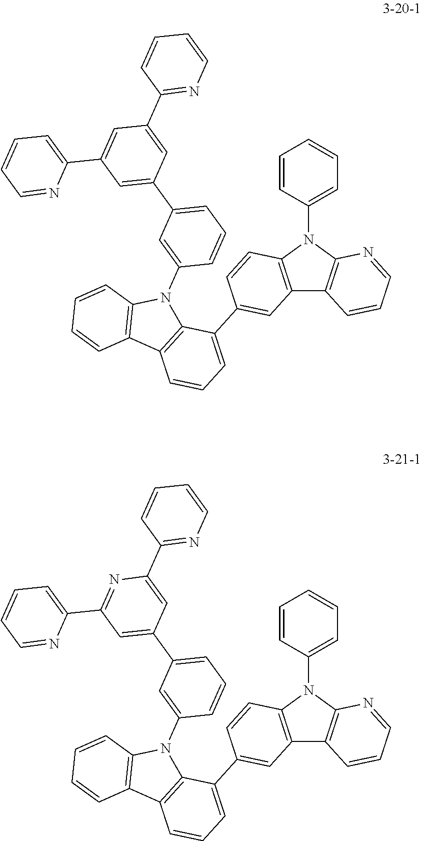 [SMILES: c1ccc(-n2c3ccc(-c4cccc5c6ccccc6n(-c6cccc(-c7cc(-c8ccccn8)cc(-c8ccccn8)c7)c6)c45)cc3c3cccnc32)cc1 ; c1ccc(-n2c3ccc(-c4cccc5c6ccccc6n(-c6cccc(-c7cc(-c8ccccn8)nc(-c8ccccn8)c7)c6)c45)cc3c3cccnc32)cc1]

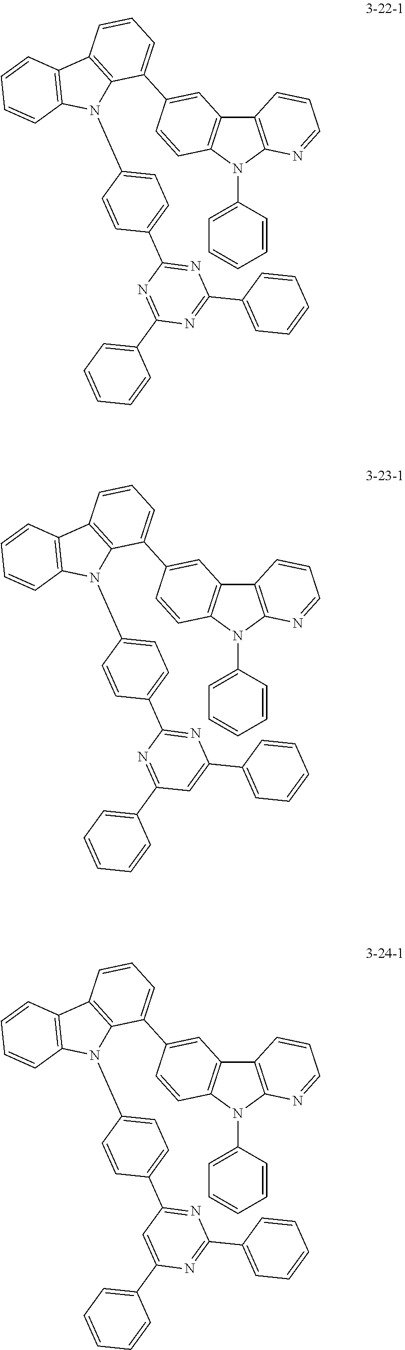 [SMILES: c1ccc(-c2nc(-c3ccccc3)nc(-c3ccc(-n4c5ccccc5c5cccc(-c6ccc7c(c6)c6cccnc6n7-c6ccccc6)c54)cc3)n2)cc1 ; c1ccc(-c2cc(-c3ccccc3)nc(-c3ccc(-n4c5ccccc5c5cccc(-c6ccc7c(c6)c6cccnc6n7-c6ccccc6)c54)cc3)n2)cc1 ; c1ccc(-c2cc(-c3ccc(-n4c5ccccc5c5cccc(-c6ccc7c(c6)c6cccnc6n7-c6ccccc6)c54)cc3)nc(-c3ccccc3)n2)cc1]

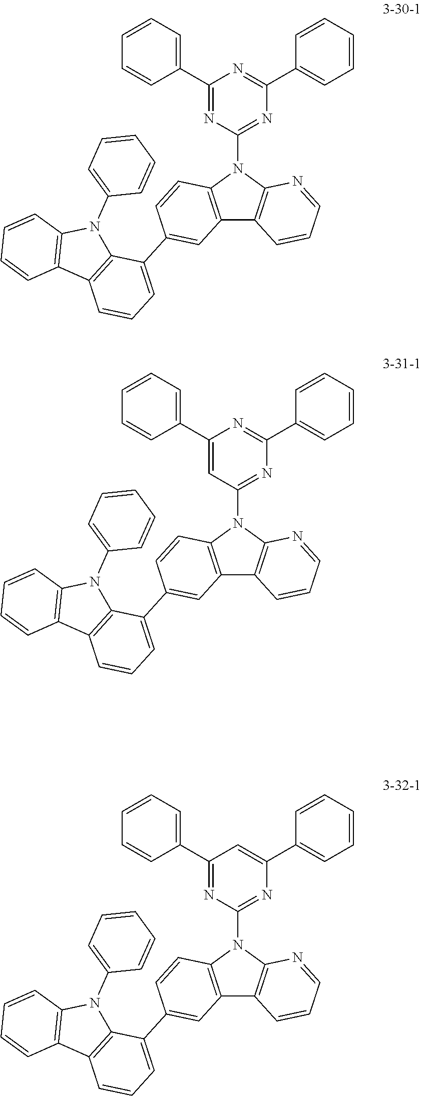 [SMILES: c1ccc(-c2nc(-c3ccccc3)nc(-n3c4ccc(-c5cccc6c7ccccc7n(-c7ccccc7)c56)cc4c4cccnc43)n2)cc1 ; c1ccc(-c2cc(-n3c4ccc(-c5cccc6c7ccccc7n(-c7ccccc7)c56)cc4c4cccnc43)nc(-c3ccccc3)n2)cc1 ; c1ccc(-c2cc(-c3ccccc3)nc(-n3c4ccc(-c5cccc6c7ccccc7n(-c7ccccc7)c56)cc4c4cccnc43)n2)cc1]

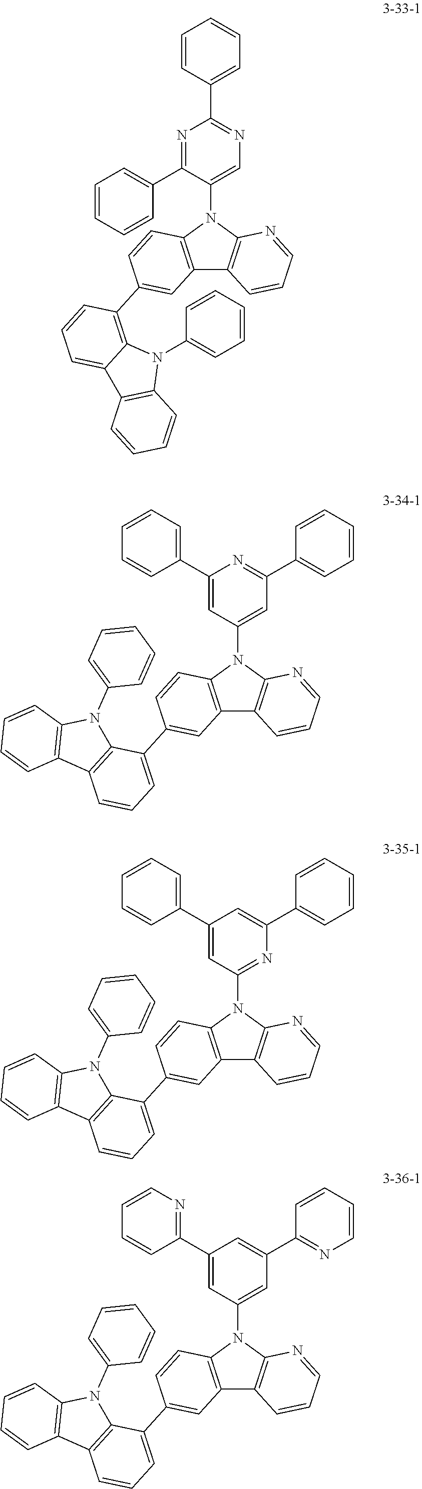

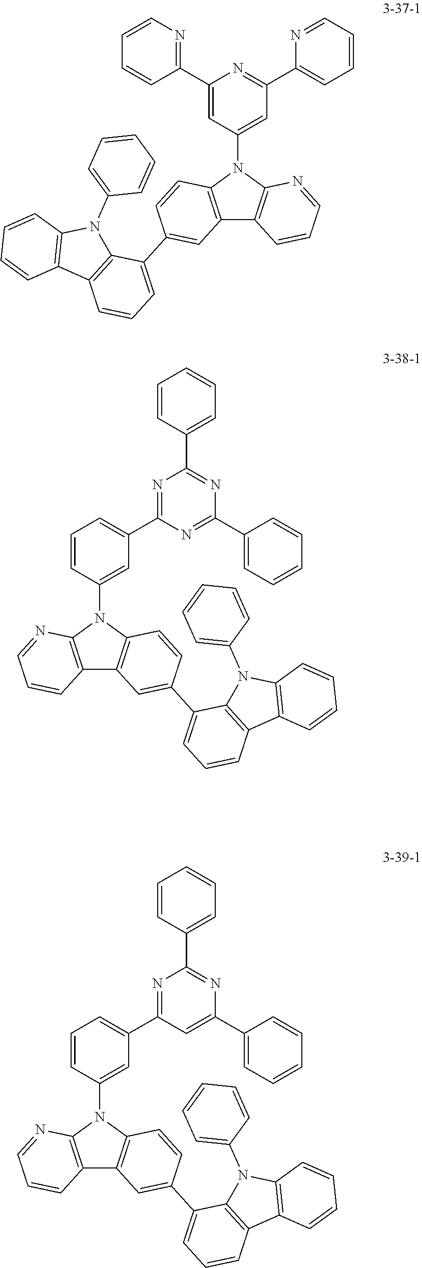 [SMILES: c1ccc(-n2c3ccccc3c3cccc(-c4ccc5c(c4)c4cccnc4n5-c4cc(-c5ccccn5)nc(-c5ccccn5)c4)c32)cc1 ; c1ccc(-c2nc(-c3ccccc3)nc(-c3cccc(-n4c5ccc(-c6cccc7c8ccccc8n(-c8ccccc8)c67)cc5c5cccnc54)c3)n2)cc1 ; c1ccc(-c2cc(-c3cccc(-n4c5ccc(-c6cccc7c8ccccc8n(-c8ccccc8)c67)cc5c5cccnc54)c3)nc(-c3ccccc3)n2)cc1]

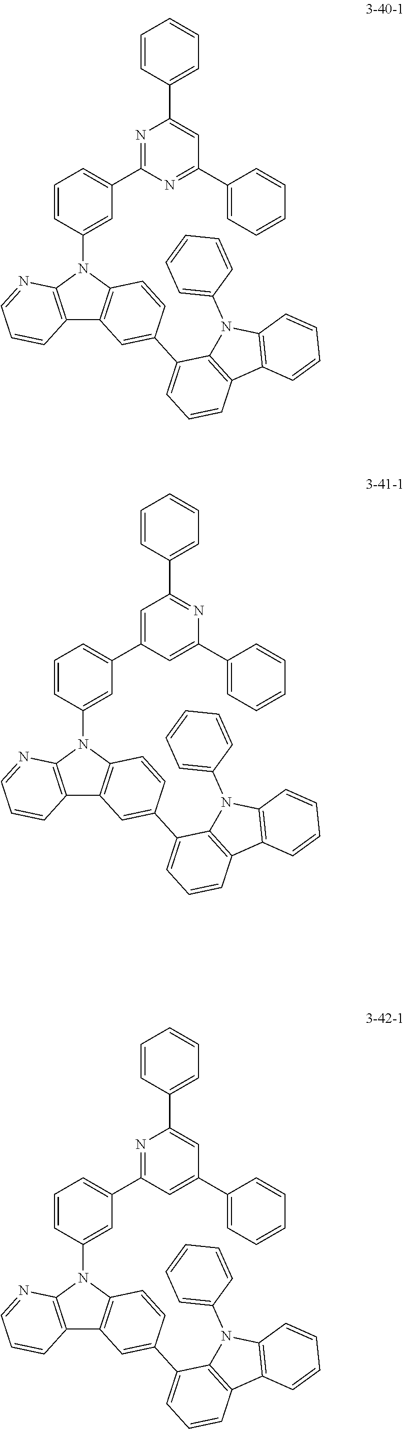 [SMILES: c1ccc(-c2cc(-c3ccccc3)nc(-c3cccc(-n4c5ccc(-c6cccc7c8ccccc8n(-c8ccccc8)c67)cc5c5cccnc54)c3)n2)cc1 ; c1ccc(-c2cc(-c3cccc(-n4c5ccc(-c6cccc7c8ccccc8n(-c8ccccc8)c67)cc5c5cccnc54)c3)cc(-c3ccccc3)n2)cc1 ; c1ccc(-c2cc(-c3ccccc3)nc(-c3cccc(-n4c5ccc(-c6cccc7c8ccccc8n(-c8ccccc8)c67)cc5c5cccnc54)c3)c2)cc1]

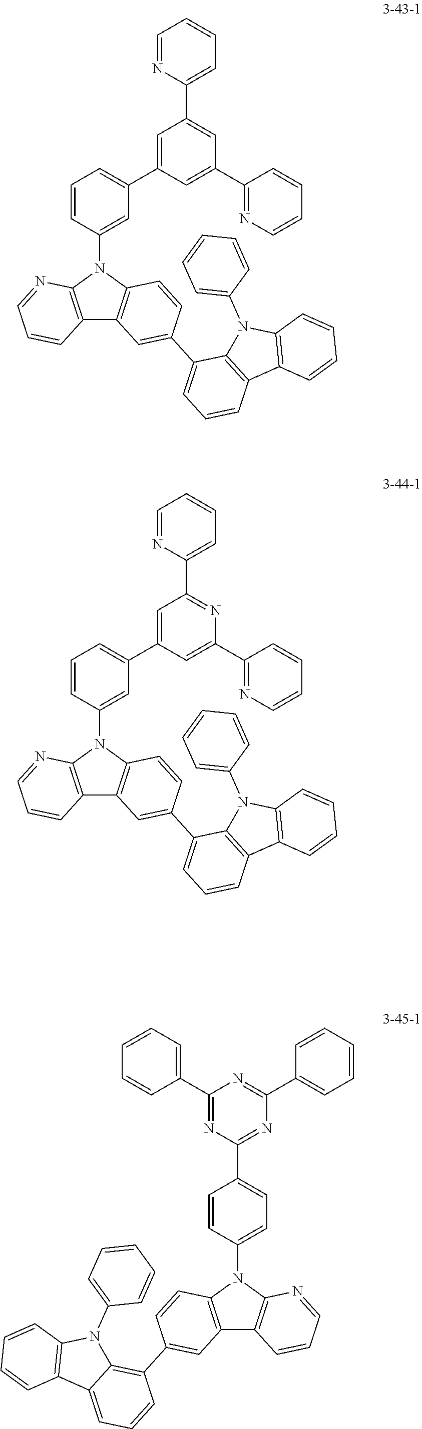 [SMILES: c1ccc(-n2c3ccccc3c3cccc(-c4ccc5c(c4)c4cccnc4n5-c4cccc(-c5cc(-c6ccccn6)cc(-c6ccccn6)c5)c4)c32)cc1 ; c1ccc(-n2c3ccccc3c3cccc(-c4ccc5c(c4)c4cccnc4n5-c4cccc(-c5cc(-c6ccccn6)nc(-c6ccccn6)c5)c4)c32)cc1 ; c1ccc(-c2nc(-c3ccccc3)nc(-c3ccc(-n4c5ccc(-c6cccc7c8ccccc8n(-c8ccccc8)c67)cc5c5cccnc54)cc3)n2)cc1]

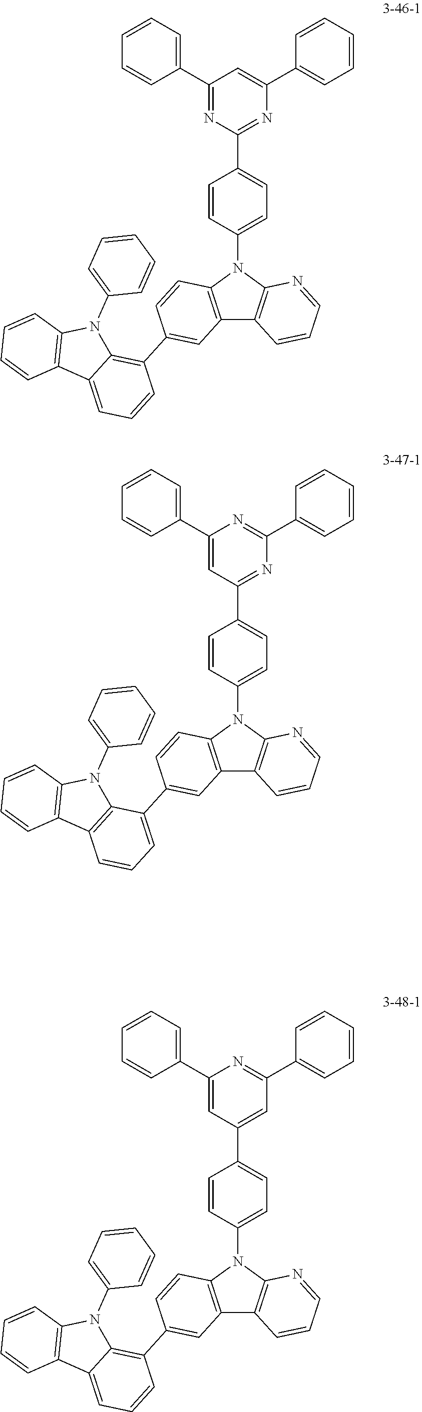 [SMILES: c1ccc(-c2cc(-c3ccccc3)nc(-c3ccc(-n4c5ccc(-c6cccc7c8ccccc8n(-c8ccccc8)c67)cc5c5cccnc54)cc3)n2)cc1 ; c1ccc(-c2cc(-c3ccc(-n4c5ccc(-c6cccc7c8ccccc8n(-c8ccccc8)c67)cc5c5cccnc54)cc3)nc(-c3ccccc3)n2)cc1 ; c1ccc(-c2cc(-c3ccc(-n4c5ccc(-c6cccc7c8ccccc8n(-c8ccccc8)c67)cc5c5cccnc54)cc3)cc(-c3ccccc3)n2)cc1]

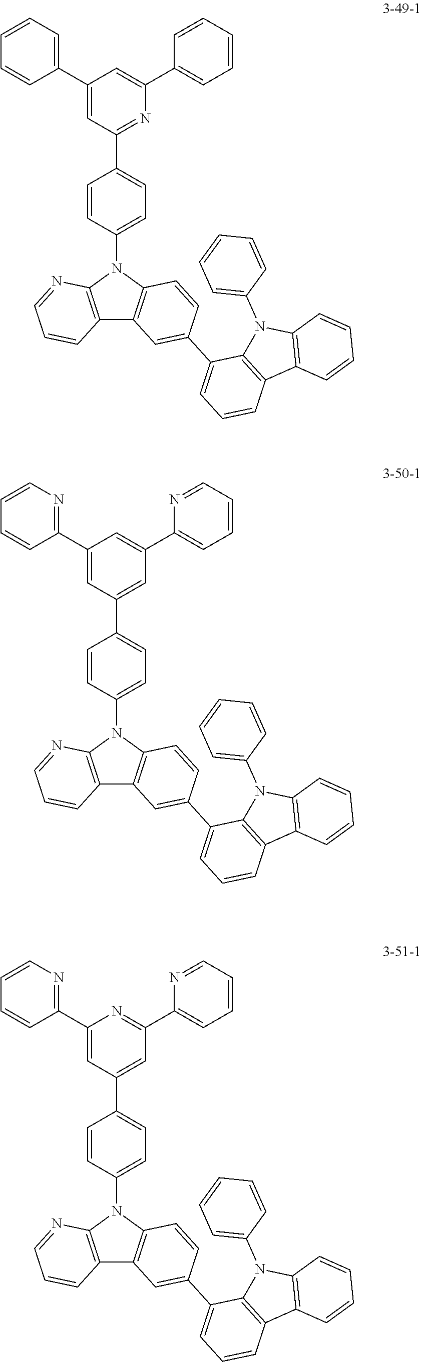 [SMILES: c1ccc(-c2cc(-c3ccccc3)nc(-c3ccc(-n4c5ccc(-c6cccc7c8ccccc8n(-c8ccccc8)c67)cc5c5cccnc54)cc3)c2)cc1 ; c1ccc(-n2c3ccccc3c3cccc(-c4ccc5c(c4)c4cccnc4n5-c4ccc(-c5cc(-c6ccccn6)cc(-c6ccccn6)c5)cc4)c32)cc1 ; c1ccc(-n2c3ccccc3c3cccc(-c4ccc5c(c4)c4cccnc4n5-c4ccc(-c5cc(-c6ccccn6)nc(-c6ccccn6)c5)cc4)c32)cc1]

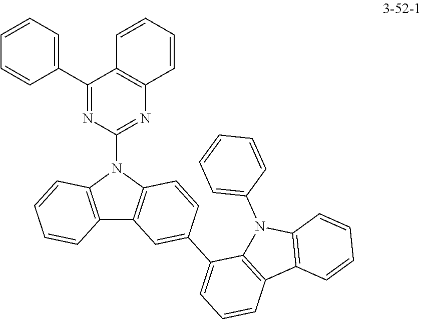 [SMILES: c1ccc(-c2nc(-n3c4ccccc4c4cc(-c5cccc6c7ccccc7n(-c7ccccc7)c56)ccc43)nc3ccccc23)cc1]

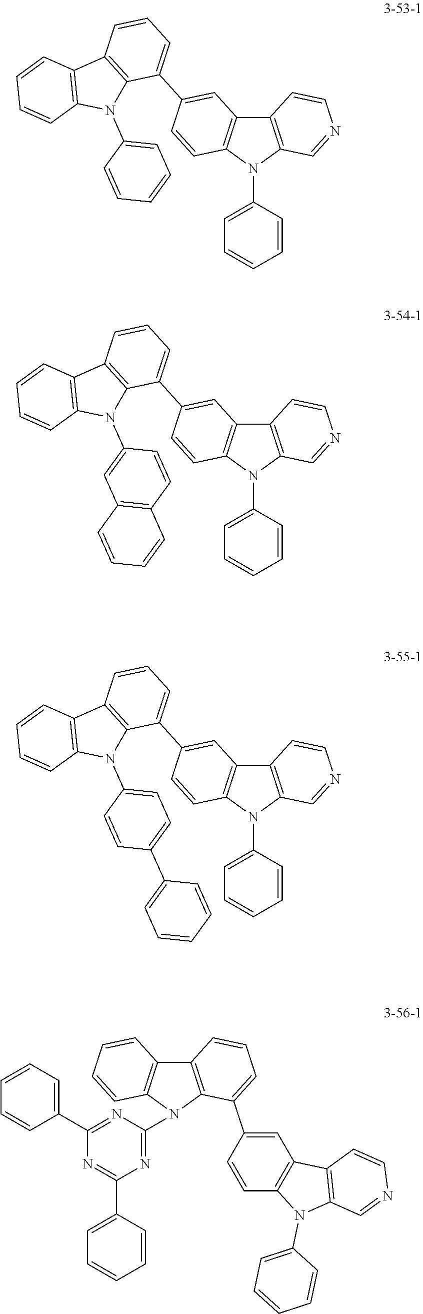 [SMILES: c1ccc(-n2c3ccc(-c4cccc5c6ccccc6n(-c6ccccc6)c45)cc3c3ccncc32)cc1 ; c1ccc(-n2c3ccc(-c4cccc5c6ccccc6n(-c6ccc7ccccc7c6)c45)cc3c3ccncc32)cc1 ; c1ccc(-c2ccc(-n3c4ccccc4c4cccc(-c5ccc6c(c5)c5ccncc5n6-c5ccccc5)c43)cc2)cc1 ; c1ccc(-c2nc(-c3ccccc3)nc(-n3c4ccccc4c4cccc(-c5ccc6c(c5)c5ccncc5n6-c5ccccc5)c43)n2)cc1]

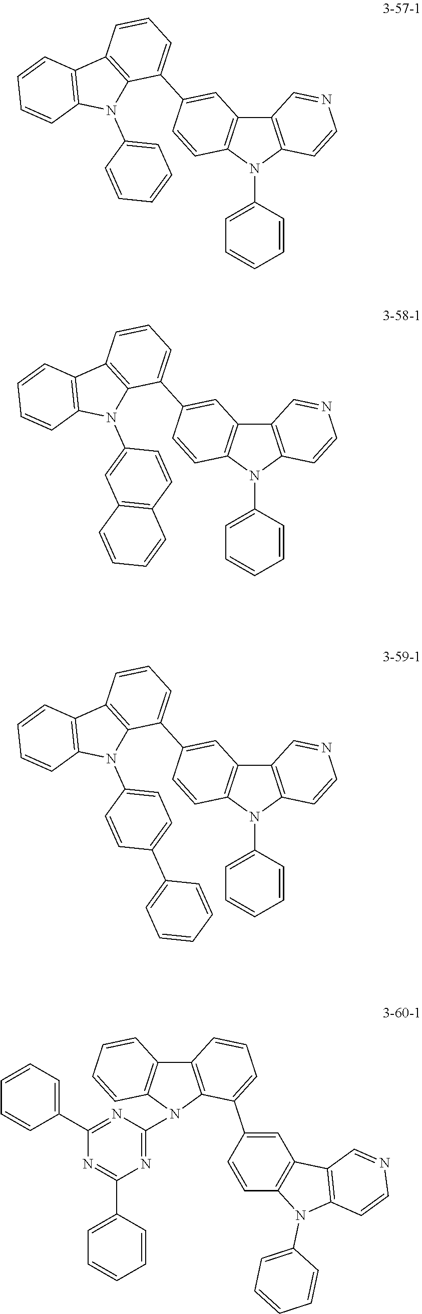 [SMILES: c1ccc(-n2c3ccncc3c3cc(-c4cccc5c6ccccc6n(-c6ccccc6)c45)ccc32)cc1 ; c1ccc(-n2c3ccncc3c3cc(-c4cccc5c6ccccc6n(-c6ccc7ccccc7c6)c45)ccc32)cc1 ; c1ccc(-c2ccc(-n3c4ccccc4c4cccc(-c5ccc6c(c5)c5cnccc5n6-c5ccccc5)c43)cc2)cc1 ; c1ccc(-c2nc(-c3ccccc3)nc(-n3c4ccccc4c4cccc(-c5ccc6c(c5)c5cnccc5n6-c5ccccc5)c43)n2)cc1]

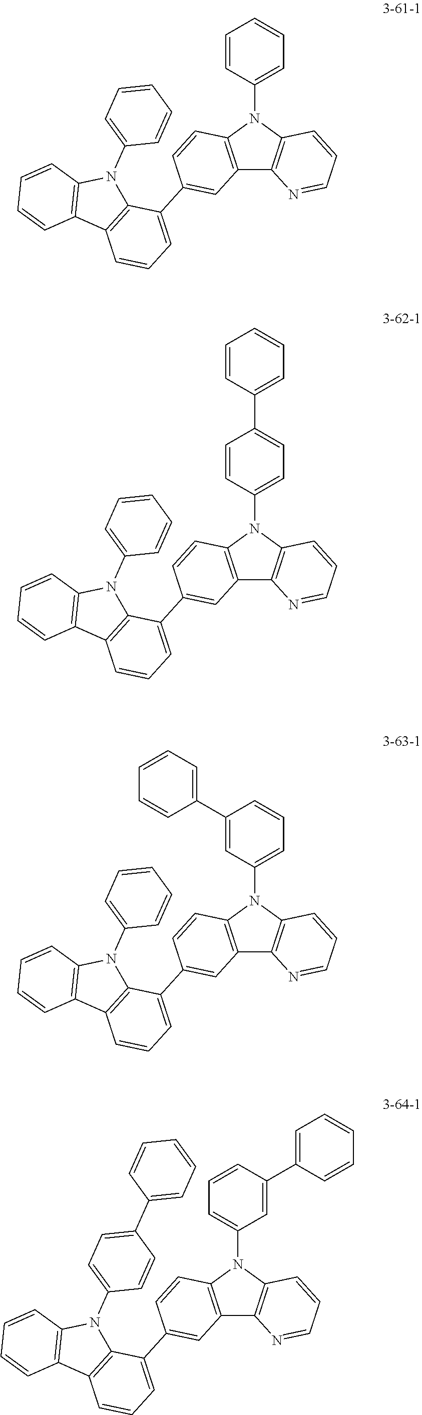 [SMILES: c1ccc(-n2c3ccc(-c4cccc5c6ccccc6n(-c6ccccc6)c45)cc3c3ncccc32)cc1 ; c1ccc(-c2ccc(-n3c4ccc(-c5cccc6c7ccccc7n(-c7ccccc7)c56)cc4c4ncccc43)cc2)cc1 ; c1ccc(-c2cccc(-n3c4ccc(-c5cccc6c7ccccc7n(-c7ccccc7)c56)cc4c4ncccc43)c2)cc1 ; c1ccc(-c2ccc(-n3c4ccccc4c4cccc(-c5ccc6c(c5)c5ncccc5n6-c5cccc(-c6ccccc6)c5)c43)cc2)cc1]

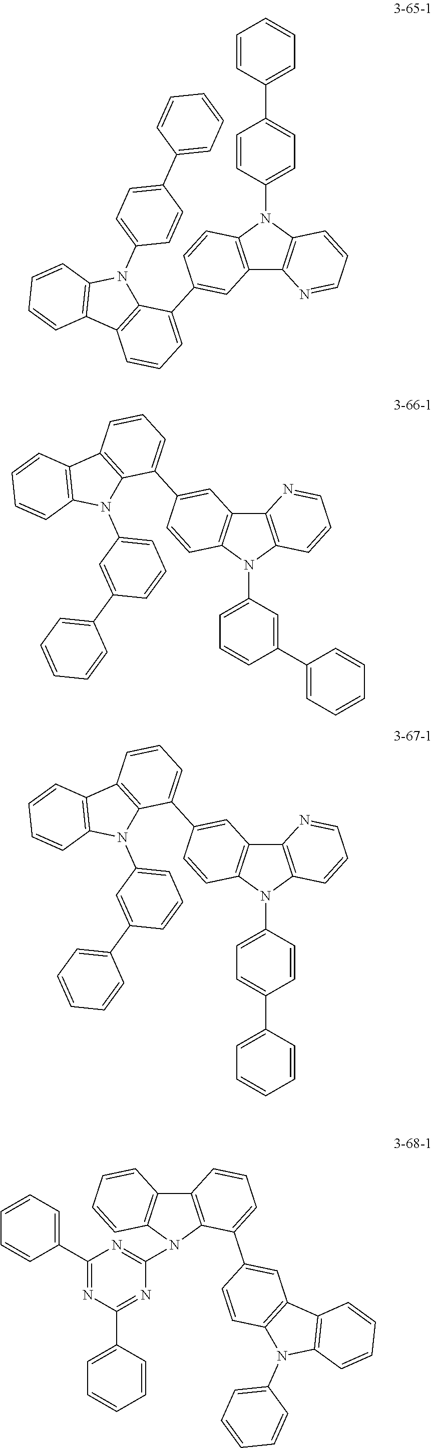 [SMILES: c1ccc(-c2ccc(-n3c4ccc(-c5cccc6c7ccccc7n(-c7ccc(-c8ccccc8)cc7)c56)cc4c4ncccc43)cc2)cc1 ; c1ccc(-c2cccc(-n3c4ccc(-c5cccc6c7ccccc7n(-c7cccc(-c8ccccc8)c7)c56)cc4c4ncccc43)c2)cc1 ; c1ccc(-c2ccc(-n3c4ccc(-c5cccc6c7ccccc7n(-c7cccc(-c8ccccc8)c7)c56)cc4c4ncccc43)cc2)cc1 ; c1ccc(-c2nc(-c3ccccc3)nc(-n3c4ccccc4c4cccc(-c5ccc6c(c5)c5ccccc5n6-c5ccccc5)c43)n2)cc1]

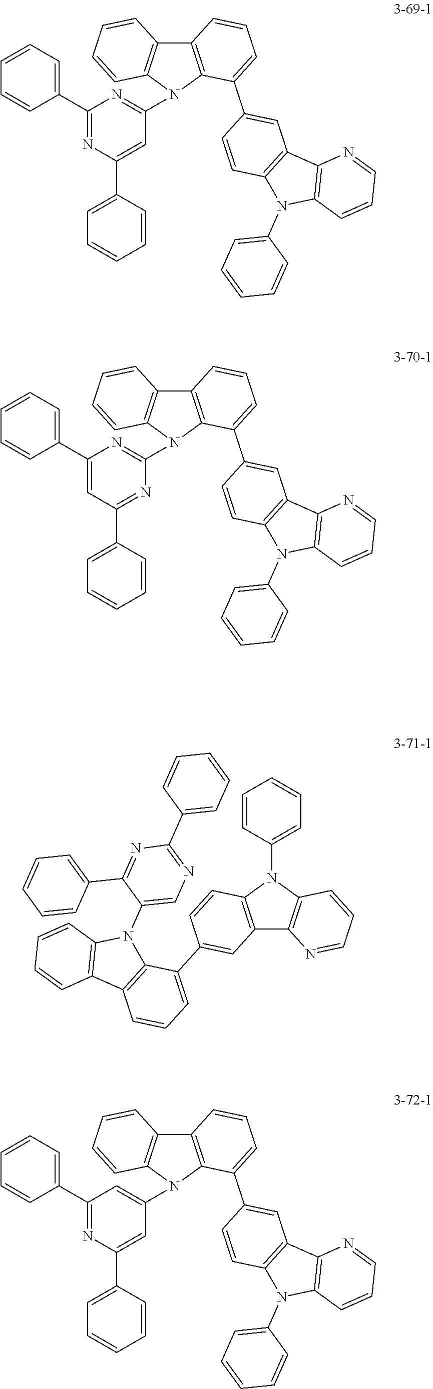 [SMILES: c1ccc(-c2cc(-n3c4ccccc4c4cccc(-c5ccc6c(c5)c5ncccc5n6-c5ccccc5)c43)nc(-c3ccccc3)n2)cc1 ; c1ccc(-c2cc(-c3ccccc3)nc(-n3c4ccccc4c4cccc(-c5ccc6c(c5)c5ncccc5n6-c5ccccc5)c43)n2)cc1 ; c1ccc(-c2ncc(-n3c4ccccc4c4cccc(-c5ccc6c(c5)c5ncccc5n6-c5ccccc5)c43)c(-c3ccccc3)n2)cc1 ; c1ccc(-c2cc(-n3c4ccccc4c4cccc(-c5ccc6c(c5)c5ncccc5n6-c5ccccc5)c43)cc(-c3ccccc3)n2)cc1]

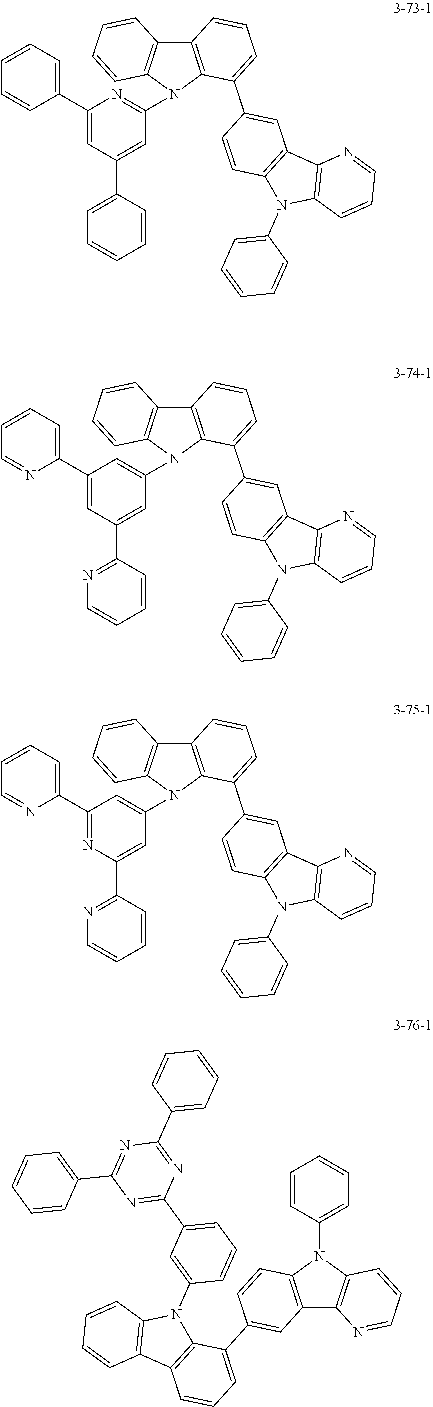 [SMILES: c1ccc(-c2cc(-c3ccccc3)nc(-n3c4ccccc4c4cccc(-c5ccc6c(c5)c5ncccc5n6-c5ccccc5)c43)c2)cc1 ; c1ccc(-n2c3ccc(-c4cccc5c6ccccc6n(-c6cc(-c7ccccn7)cc(-c7ccccn7)c6)c45)cc3c3ncccc32)cc1 ; c1ccc(-n2c3ccc(-c4cccc5c6ccccc6n(-c6cc(-c7ccccn7)nc(-c7ccccn7)c6)c45)cc3c3ncccc32)cc1 ; c1ccc(-c2nc(-c3ccccc3)nc(-c3cccc(-n4c5ccccc5c5cccc(-c6ccc7c(c6)c6ncccc6n7-c6ccccc6)c54)c3)n2)cc1]

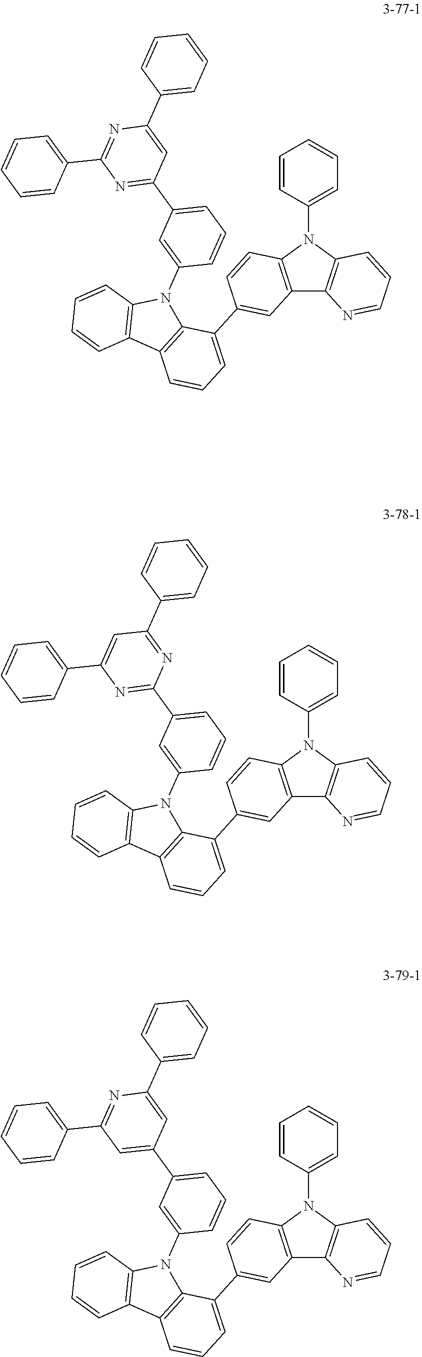 [SMILES: c1ccc(-c2cc(-c3cccc(-n4c5ccccc5c5cccc(-c6ccc7c(c6)c6ncccc6n7-c6ccccc6)c54)c3)nc(-c3ccccc3)n2)cc1 ; c1ccc(-c2cc(-c3ccccc3)nc(-c3cccc(-n4c5ccccc5c5cccc(-c6ccc7c(c6)c6ncccc6n7-c6ccccc6)c54)c3)n2)cc1 ; c1ccc(-c2cc(-c3cccc(-n4c5ccccc5c5cccc(-c6ccc7c(c6)c6ncccc6n7-c6ccccc6)c54)c3)cc(-c3ccccc3)n2)cc1]

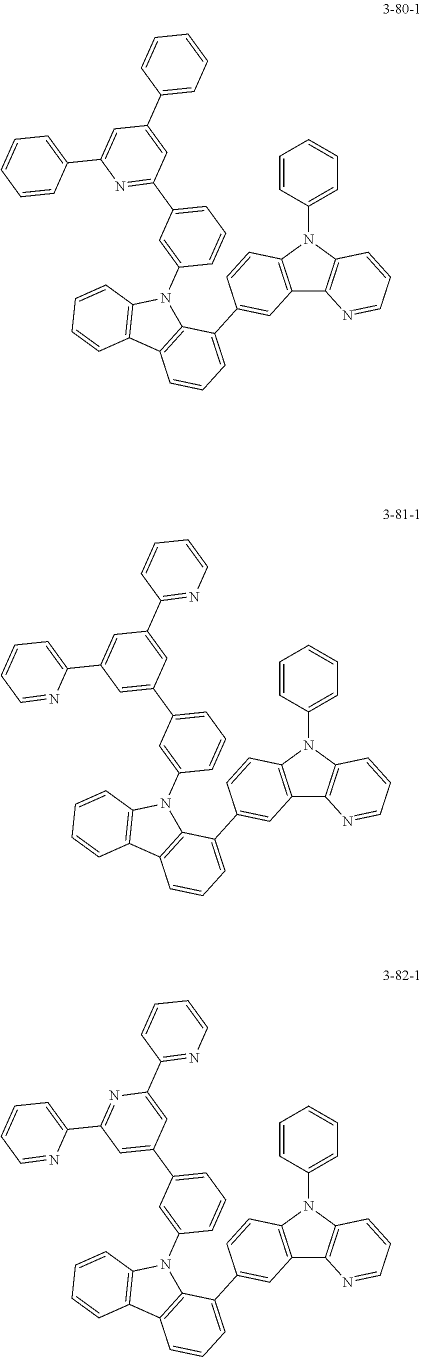 [SMILES: c1ccc(-c2cc(-c3ccccc3)nc(-c3cccc(-n4c5ccccc5c5cccc(-c6ccc7c(c6)c6ncccc6n7-c6ccccc6)c54)c3)c2)cc1 ; c1ccc(-n2c3ccc(-c4cccc5c6ccccc6n(-c6cccc(-c7cc(-c8ccccn8)cc(-c8ccccn8)c7)c6)c45)cc3c3ncccc32)cc1 ; c1ccc(-n2c3ccc(-c4cccc5c6ccccc6n(-c6cccc(-c7cc(-c8ccccn8)nc(-c8ccccn8)c7)c6)c45)cc3c3ncccc32)cc1]

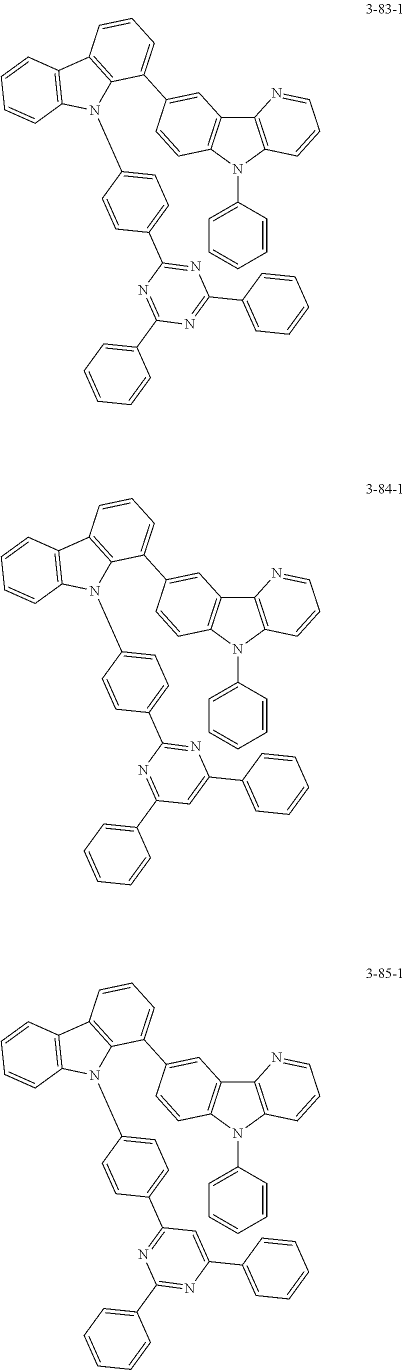 [SMILES: c1ccc(-c2nc(-c3ccccc3)nc(-c3ccc(-n4c5ccccc5c5cccc(-c6ccc7c(c6)c6ncccc6n7-c6ccccc6)c54)cc3)n2)cc1 ; c1ccc(-c2cc(-c3ccccc3)nc(-c3ccc(-n4c5ccccc5c5cccc(-c6ccc7c(c6)c6ncccc6n7-c6ccccc6)c54)cc3)n2)cc1 ; c1ccc(-c2cc(-c3ccc(-n4c5ccccc5c5cccc(-c6ccc7c(c6)c6ncccc6n7-c6ccccc6)c54)cc3)nc(-c3ccccc3)n2)cc1]

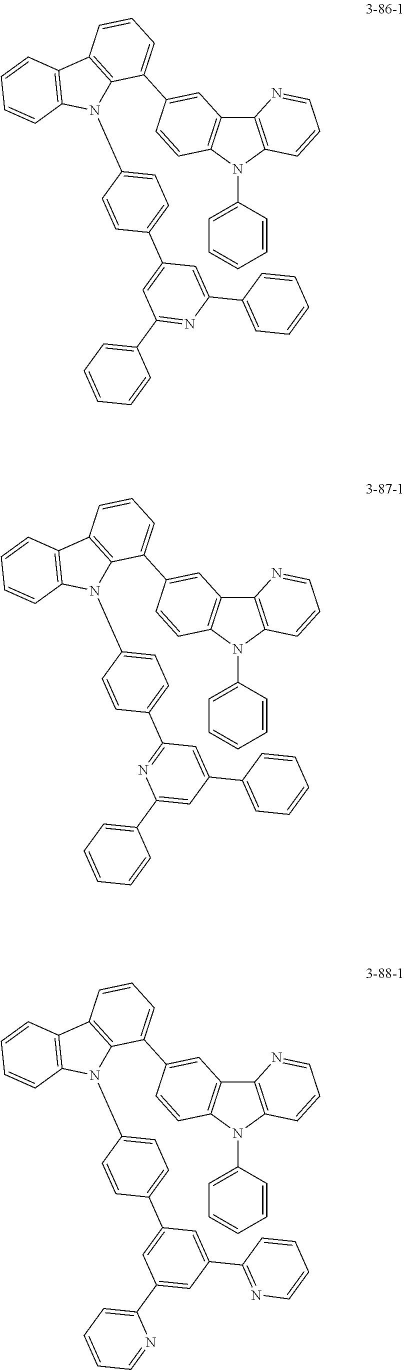 [SMILES: c1ccc(-c2cc(-c3ccc(-n4c5ccccc5c5cccc(-c6ccc7c(c6)c6ncccc6n7-c6ccccc6)c54)cc3)cc(-c3ccccc3)n2)cc1 ; c1ccc(-c2cc(-c3ccccc3)nc(-c3ccc(-n4c5ccccc5c5cccc(-c6ccc7c(c6)c6ncccc6n7-c6ccccc6)c54)cc3)c2)cc1 ; c1ccc(-n2c3ccc(-c4cccc5c6ccccc6n(-c6ccc(-c7cc(-c8ccccn8)cc(-c8ccccn8)c7)cc6)c45)cc3c3ncccc32)cc1]

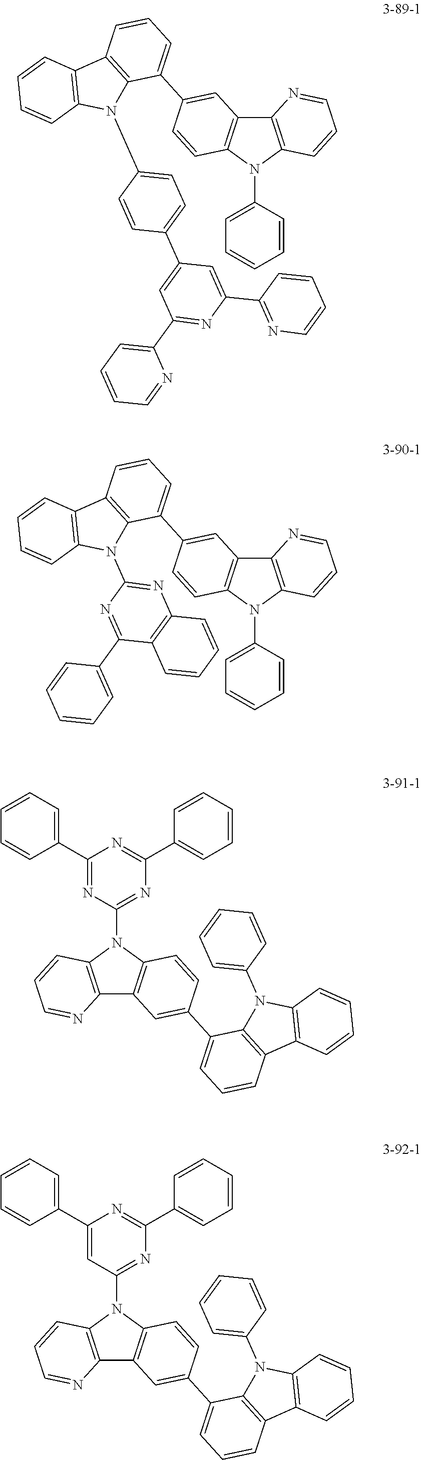 [SMILES: c1ccc(-n2c3ccc(-c4cccc5c6ccccc6n(-c6ccc(-c7cc(-c8ccccn8)nc(-c8ccccn8)c7)cc6)c45)cc3c3ncccc32)cc1 ; c1ccc(-c2nc(-n3c4ccccc4c4cccc(-c5ccc6c(c5)c5ncccc5n6-c5ccccc5)c43)nc3ccccc23)cc1 ; c1ccc(-c2nc(-c3ccccc3)nc(-n3c4ccc(-c5cccc6c7ccccc7n(-c7ccccc7)c56)cc4c4ncccc43)n2)cc1 ; c1ccc(-c2cc(-n3c4ccc(-c5cccc6c7ccccc7n(-c7ccccc7)c56)cc4c4ncccc43)nc(-c3ccccc3)n2)cc1]

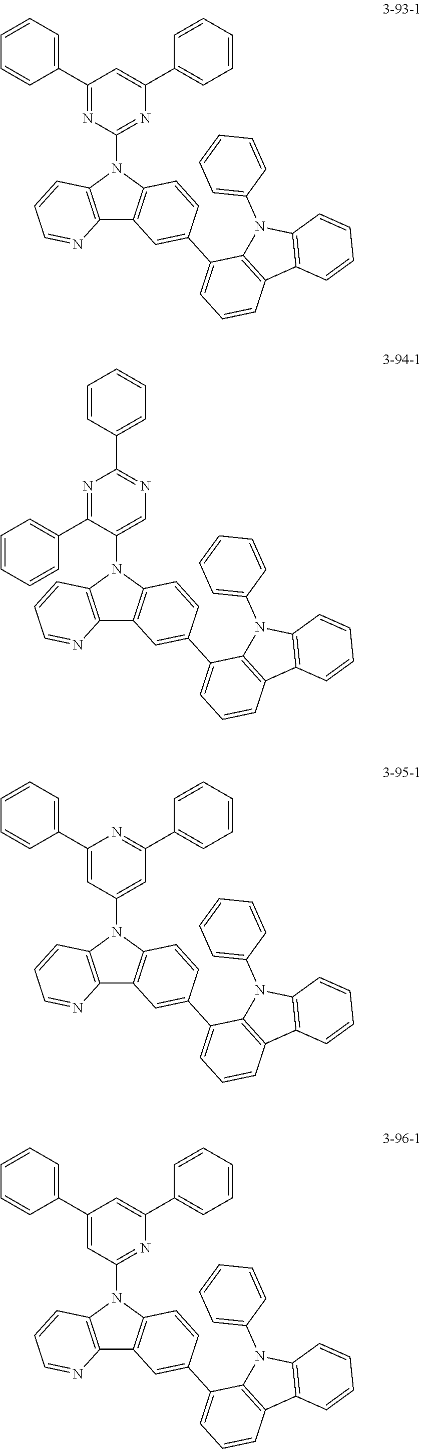 [SMILES: c1ccc(-c2cc(-c3ccccc3)nc(-n3c4ccc(-c5cccc6c7ccccc7n(-c7ccccc7)c56)cc4c4ncccc43)n2)cc1 ; c1ccc(-c2ncc(-n3c4ccc(-c5cccc6c7ccccc7n(-c7ccccc7)c56)cc4c4ncccc43)c(-c3ccccc3)n2)cc1 ; c1ccc(-c2cc(-n3c4ccc(-c5cccc6c7ccccc7n(-c7ccccc7)c56)cc4c4ncccc43)cc(-c3ccccc3)n2)cc1 ; c1ccc(-c2cc(-c3ccccc3)nc(-n3c4ccc(-c5cccc6c7ccccc7n(-c7ccccc7)c56)cc4c4ncccc43)c2)cc1]

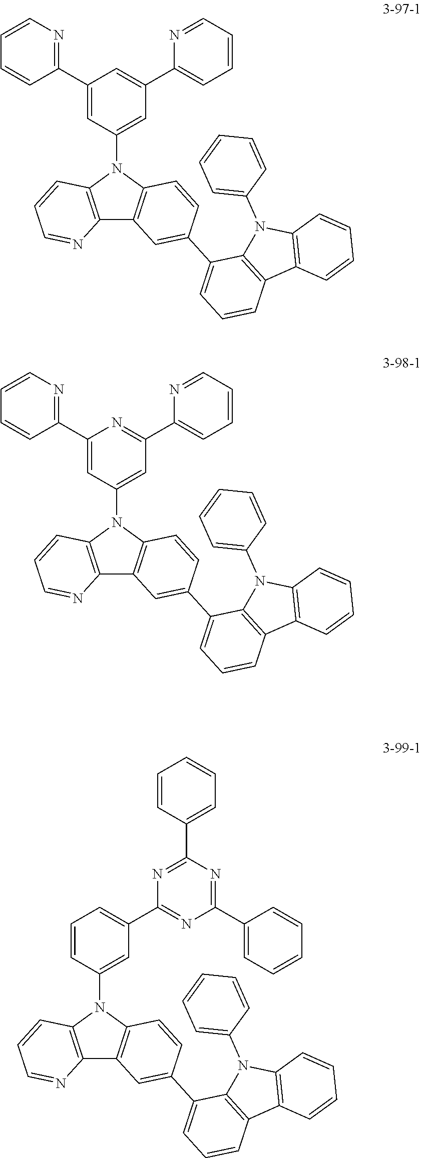 [SMILES: c1ccc(-n2c3ccccc3c3cccc(-c4ccc5c(c4)c4ncccc4n5-c4cc(-c5ccccn5)cc(-c5ccccn5)c4)c32)cc1 ; c1ccc(-n2c3ccccc3c3cccc(-c4ccc5c(c4)c4ncccc4n5-c4cc(-c5ccccn5)nc(-c5ccccn5)c4)c32)cc1 ; c1ccc(-c2nc(-c3ccccc3)nc(-c3cccc(-n4c5ccc(-c6cccc7c8ccccc8n(-c8ccccc8)c67)cc5c5ncccc54)c3)n2)cc1]

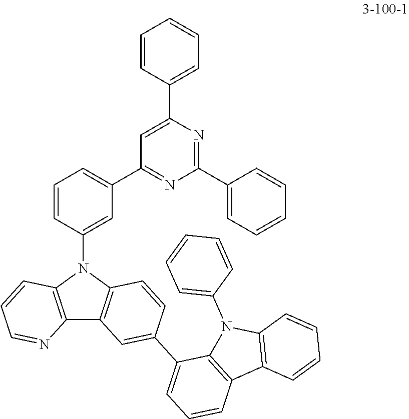 [SMILES: c1ccc(-c2cc(-c3cccc(-n4c5ccc(-c6cccc7c8ccccc8n(-c8ccccc8)c67)cc5c5ncccc54)c3)nc(-c3ccccc3)n2)cc1]

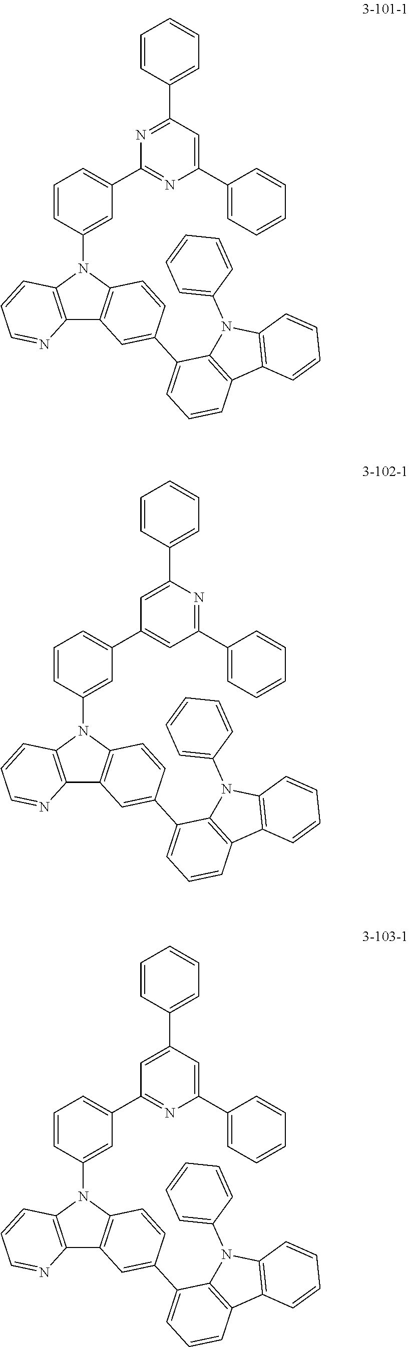 [SMILES: c1ccc(-c2cc(-c3ccccc3)nc(-c3cccc(-n4c5ccc(-c6cccc7c8ccccc8n(-c8ccccc8)c67)cc5c5ncccc54)c3)n2)cc1 ; c1ccc(-c2cc(-c3cccc(-n4c5ccc(-c6cccc7c8ccccc8n(-c8ccccc8)c67)cc5c5ncccc54)c3)cc(-c3ccccc3)n2)cc1 ; c1ccc(-c2cc(-c3ccccc3)nc(-c3cccc(-n4c5ccc(-c6cccc7c8ccccc8n(-c8ccccc8)c67)cc5c5ncccc54)c3)c2)cc1]

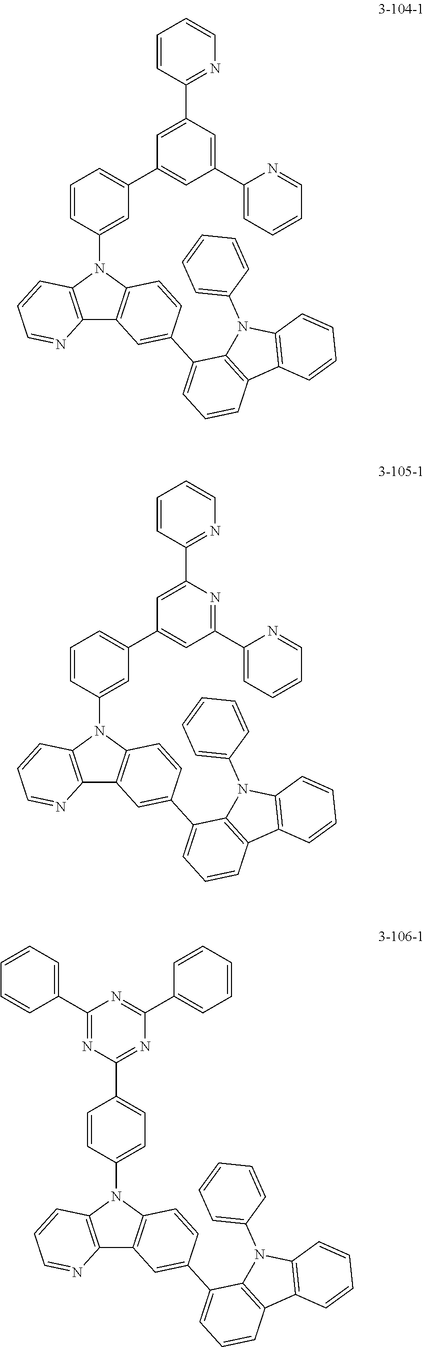 [SMILES: c1ccc(-n2c3ccccc3c3cccc(-c4ccc5c(c4)c4ncccc4n5-c4cccc(-c5cc(-c6ccccn6)cc(-c6ccccn6)c5)c4)c32)cc1 ; c1ccc(-n2c3ccccc3c3cccc(-c4ccc5c(c4)c4ncccc4n5-c4cccc(-c5cc(-c6ccccn6)nc(-c6ccccn6)c5)c4)c32)cc1 ; c1ccc(-c2nc(-c3ccccc3)nc(-c3ccc(-n4c5ccc(-c6cccc7c8ccccc8n(-c8ccccc8)c67)cc5c5ncccc54)cc3)n2)cc1]

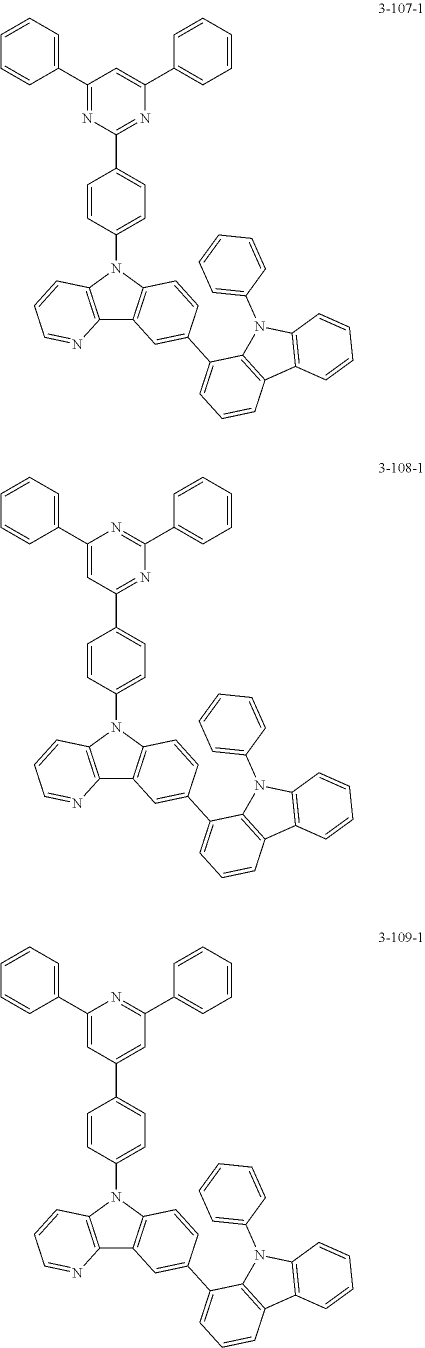 [SMILES: c1ccc(-c2cc(-c3ccccc3)nc(-c3ccc(-n4c5ccc(-c6cccc7c8ccccc8n(-c8ccccc8)c67)cc5c5ncccc54)cc3)n2)cc1 ; c1ccc(-c2cc(-c3ccc(-n4c5ccc(-c6cccc7c8ccccc8n(-c8ccccc8)c67)cc5c5ncccc54)cc3)nc(-c3ccccc3)n2)cc1 ; c1ccc(-c2cc(-c3ccc(-n4c5ccc(-c6cccc7c8ccccc8n(-c8ccccc8)c67)cc5c5ncccc54)cc3)cc(-c3ccccc3)n2)cc1]

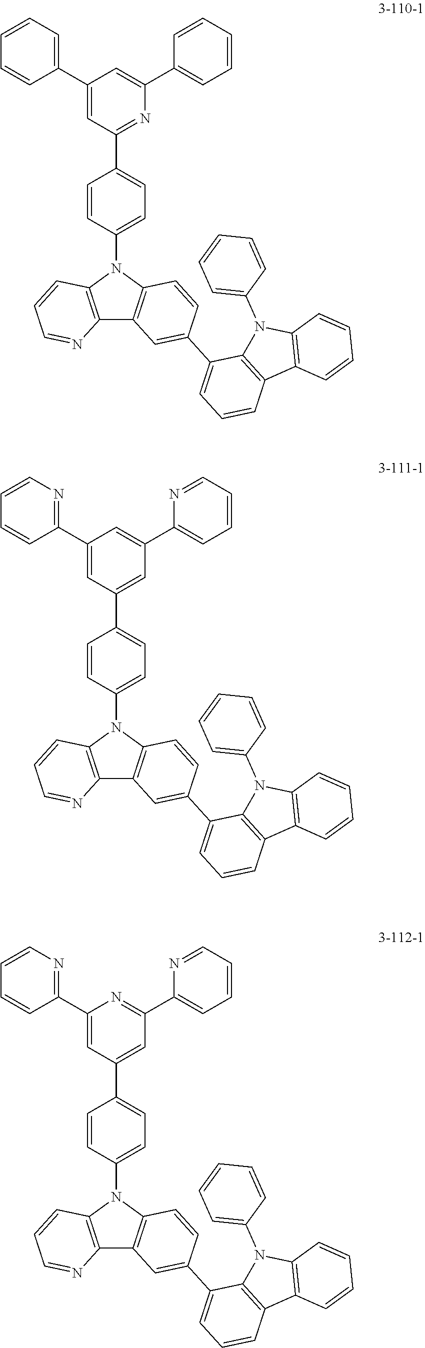 [SMILES: c1ccc(-c2cc(-c3ccccc3)nc(-c3ccc(-n4c5ccc(-c6cccc7c8ccccc8n(-c8ccccc8)c67)cc5c5ncccc54)cc3)c2)cc1 ; c1ccc(-n2c3ccccc3c3cccc(-c4ccc5c(c4)c4ncccc4n5-c4ccc(-c5cc(-c6ccccn6)cc(-c6ccccn6)c5)cc4)c32)cc1 ; c1ccc(-n2c3ccccc3c3cccc(-c4ccc5c(c4)c4ncccc4n5-c4ccc(-c5cc(-c6ccccn6)nc(-c6ccccn6)c5)cc4)c32)cc1]

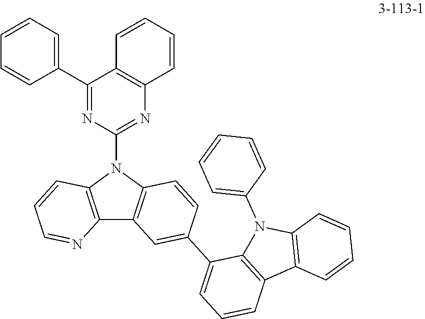 [SMILES: c1ccc(-c2nc(-n3c4ccc(-c5cccc6c7ccccc7n(-c7ccccc7)c56)cc4c4ncccc43)nc3ccccc23)cc1]

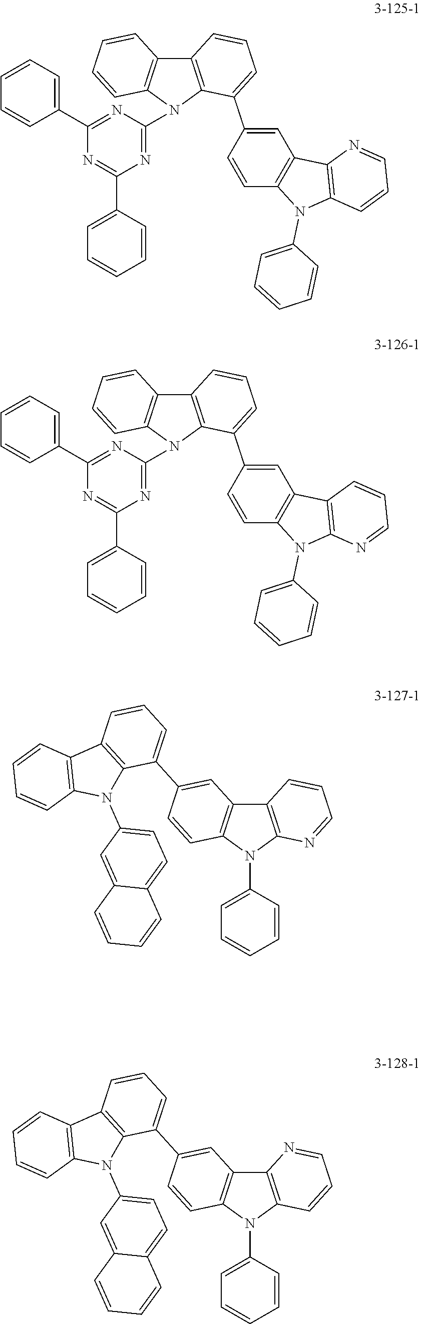 [SMILES: c1ccc(-c2nc(-c3ccccc3)nc(-n3c4ccccc4c4cccc(-c5ccc6c(c5)c5ncccc5n6-c5ccccc5)c43)n2)cc1 ; c1ccc(-c2nc(-c3ccccc3)nc(-n3c4ccccc4c4cccc(-c5ccc6c(c5)c5cccnc5n6-c5ccccc5)c43)n2)cc1 ; c1ccc(-n2c3ccc(-c4cccc5c6ccccc6n(-c6ccc7ccccc7c6)c45)cc3c3cccnc32)cc1 ; c1ccc(-n2c3ccc(-c4cccc5c6ccccc6n(-c6ccc7ccccc7c6)c45)cc3c3ncccc32)cc1]

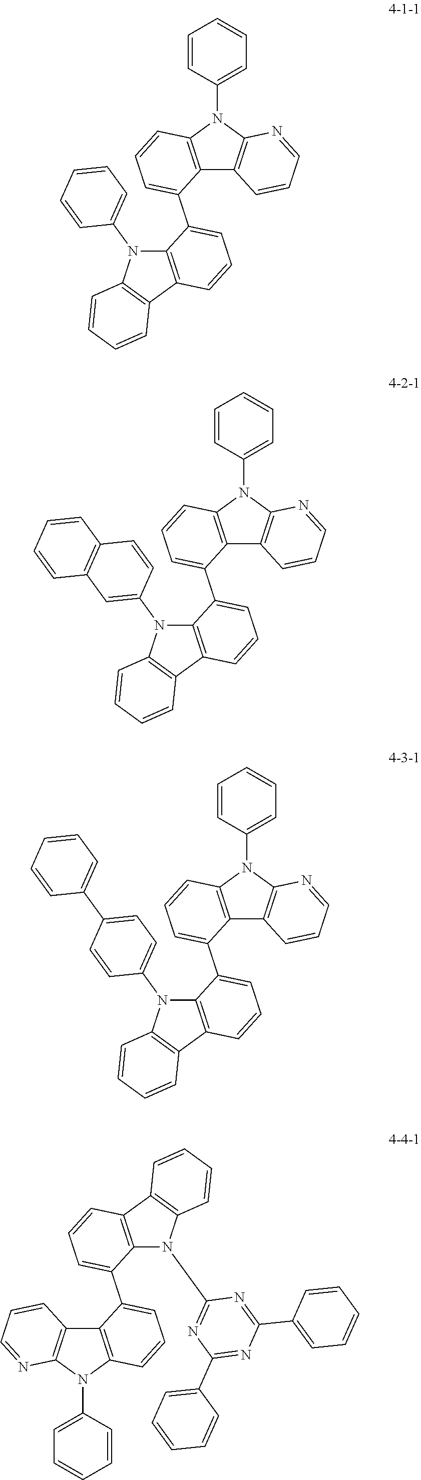 [SMILES: c1ccc(-n2c3cccc(-c4cccc5c6ccccc6n(-c6ccccc6)c45)c3c3cccnc32)cc1 ; c1ccc(-n2c3cccc(-c4cccc5c6ccccc6n(-c6ccc7ccccc7c6)c45)c3c3cccnc32)cc1 ; c1ccc(-c2ccc(-n3c4ccccc4c4cccc(-c5cccc6c5c5cccnc5n6-c5ccccc5)c43)cc2)cc1 ; c1ccc(-c2nc(Cn3c4ccccc4c4cccc(-c5cccc6c5c5cccnc5n6-c5ccccc5)c43)nc(-c3ccccc3)n2)cc1]

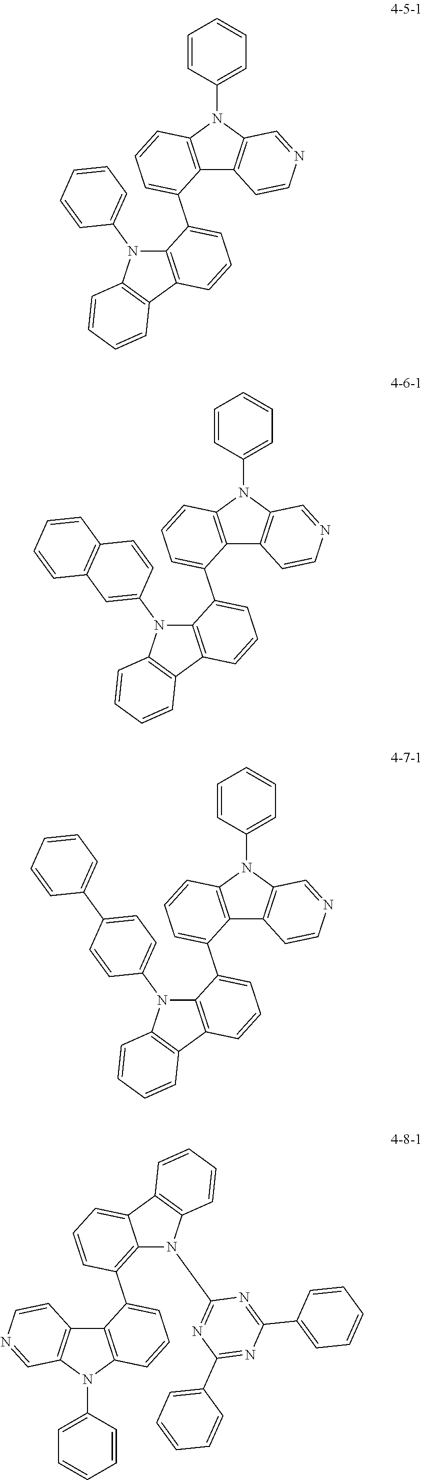 [SMILES: c1ccc(-n2c3cnccc3c3c(-c4cccc5c6ccccc6n(-c6ccccc6)c45)cccc32)cc1 ; c1ccc(-n2c3cnccc3c3c(-c4cccc5c6ccccc6n(-c6ccc7ccccc7c6)c45)cccc32)cc1 ; c1ccc(-c2ccc(-n3c4ccccc4c4cccc(-c5cccc6c5c5ccncc5n6-c5ccccc5)c43)cc2)cc1 ; c1ccc(-c2nc(Cn3c4ccccc4c4cccc(-c5cccc6c5c5ccncc5n6-c5ccccc5)c43)nc(-c3ccccc3)n2)cc1]

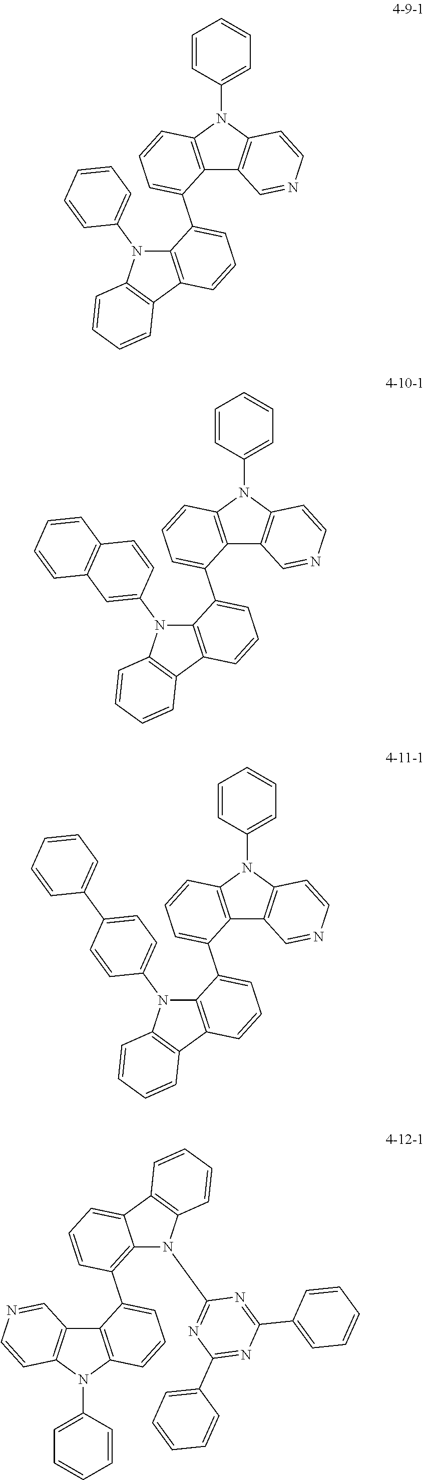 [SMILES: c1ccc(-n2c3ccncc3c3c(-c4cccc5c6ccccc6n(-c6ccccc6)c45)cccc32)cc1 ; c1ccc(-n2c3ccncc3c3c(-c4cccc5c6ccccc6n(-c6ccc7ccccc7c6)c45)cccc32)cc1 ; c1ccc(-c2ccc(-n3c4ccccc4c4cccc(-c5cccc6c5c5cnccc5n6-c5ccccc5)c43)cc2)cc1 ; c1ccc(-c2nc(Cn3c4ccccc4c4cccc(-c5cccc6c5c5cnccc5n6-c5ccccc5)c43)nc(-c3ccccc3)n2)cc1]

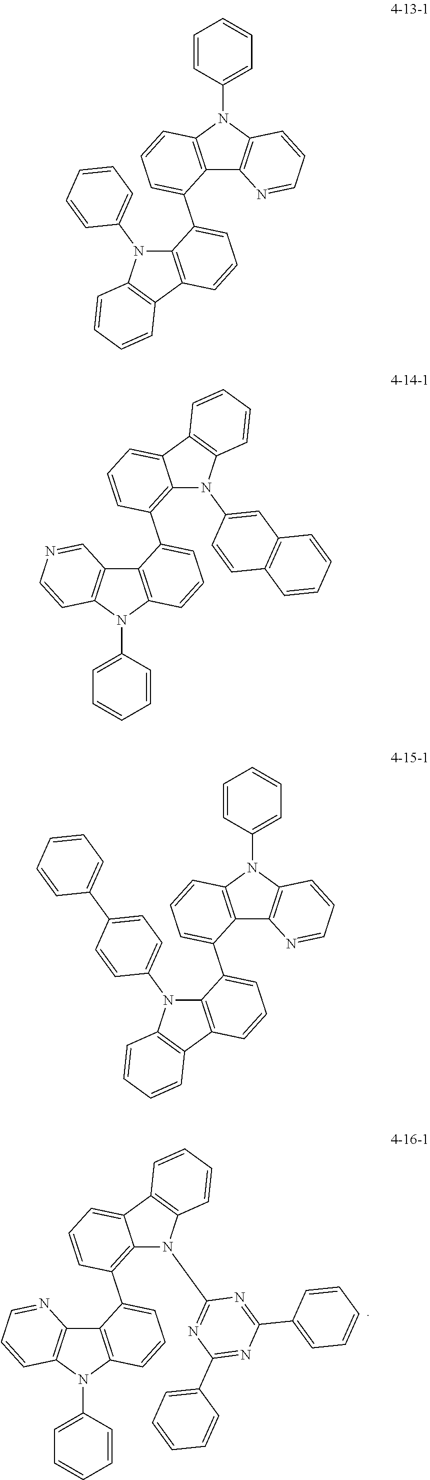 [SMILES: c1ccc(-n2c3cccnc3c3c(-c4cccc5c6ccccc6n(-c6ccccc6)c45)cccc32)cc1 ; c1ccc(-n2c3ccncc3c3c(-c4cccc5c6ccccc6n(-c6ccc7ccccc7c6)c45)cccc32)cc1 ; c1ccc(-c2ccc(-n3c4ccccc4c4cccc(-c5cccc6c5c5ncccc5n6-c5ccccc5)c43)cc2)cc1 ; c1ccc(-c2nc(Cn3c4ccccc4c4cccc(-c5cccc6c5c5ncccc5n6-c5ccccc5)c43)nc(-c3ccccc3)n2)cc1 ; "."]

4. The compound of claim 1, wherein the compound is represented by one of the formulas below: 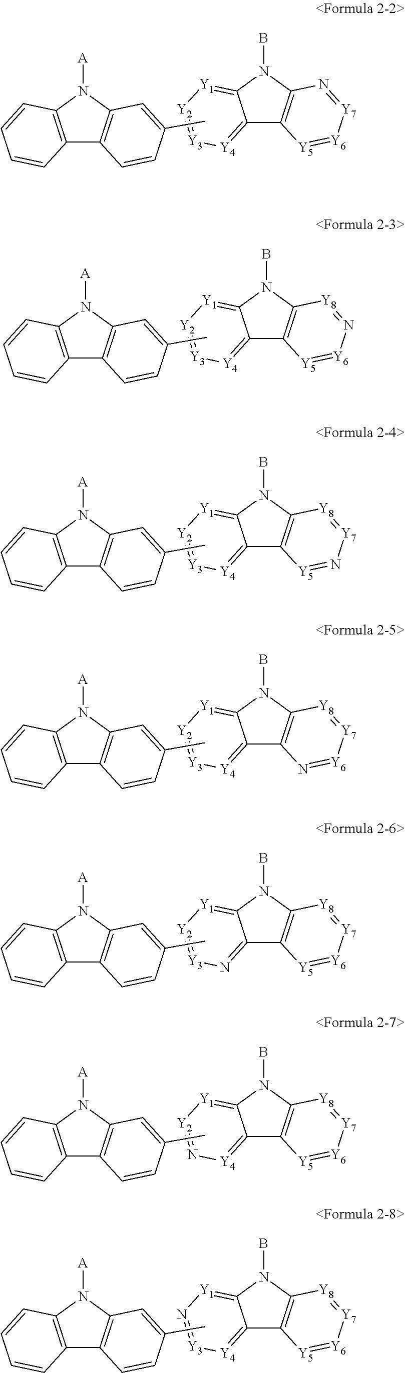 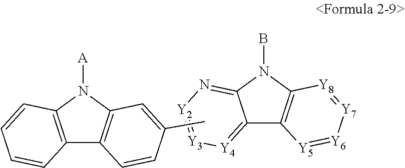
wherein in Formulas 2-2 to 2-9, Y₁ to Y₈ and A and B are identical to Y₁ to Y₈ and A and B defined in Formula 2-1.

5. The compound of claim 1, wherein the compound is represented by one of the formulas below: 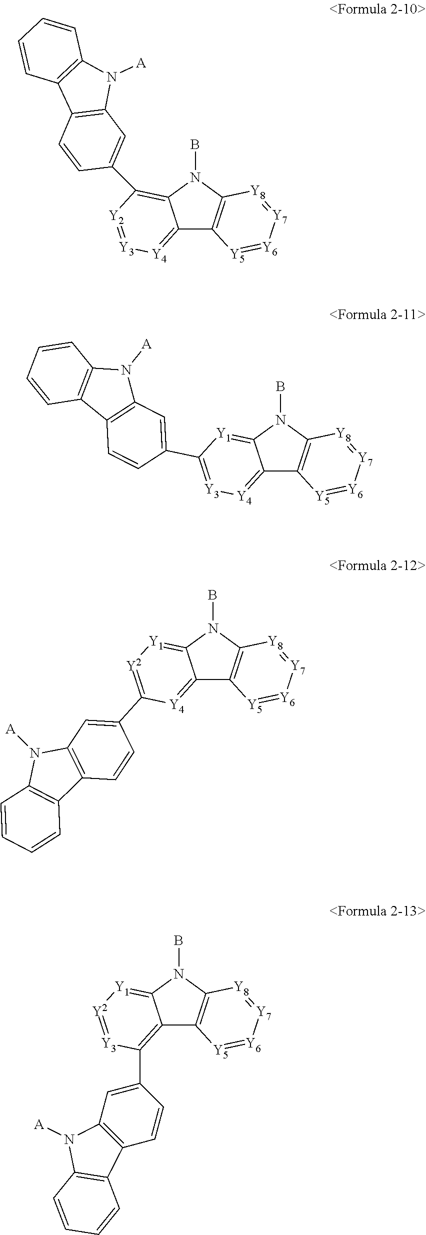
wherein in Formulas 2-10 to 2-13, Y₁ to Y₈ each are independently CH or N, and at least one of Y₁ to Y₈ is N, and A and B are identical to A and B defined in Formula 2-1.

6. The compound of claim 1, wherein the compound is represented by one of the formulas below:

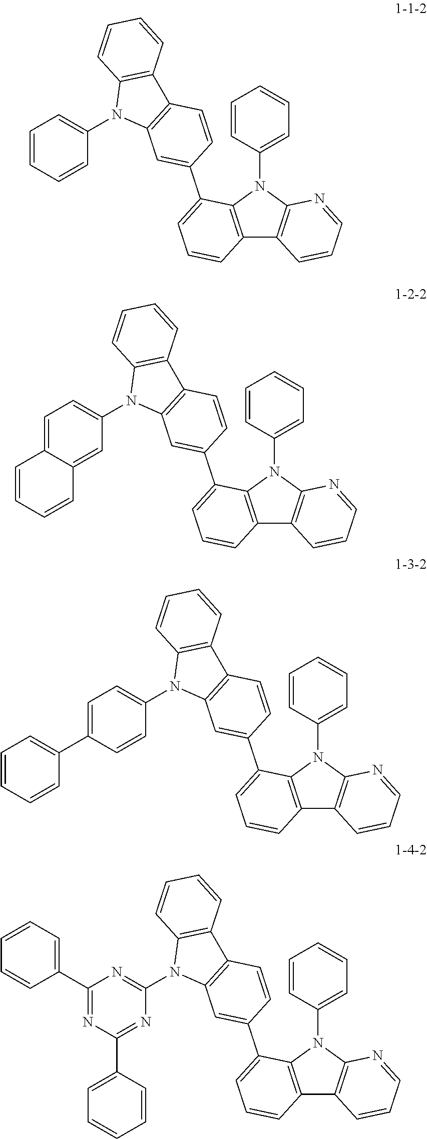 [SMILES: c1ccc(-n2c3ccccc3c3ccc(-c4cccc5c6cccnc6n(-c6ccccc6)c45)cc32)cc1 ; c1ccc(-n2c3ncccc3c3cccc(-c4ccc5c6ccccc6n(-c6ccc7ccccc7c6)c5c4)c32)cc1 ; c1ccc(-c2ccc(-n3c4ccccc4c4ccc(-c5cccc6c7cccnc7n(-c7ccccc7)c56)cc43)cc2)cc1 ; c1ccc(-c2nc(-c3ccccc3)nc(-n3c4ccccc4c4ccc(-c5cccc6c7cccnc7n(-c7ccccc7)c56)cc43)n2)cc1]

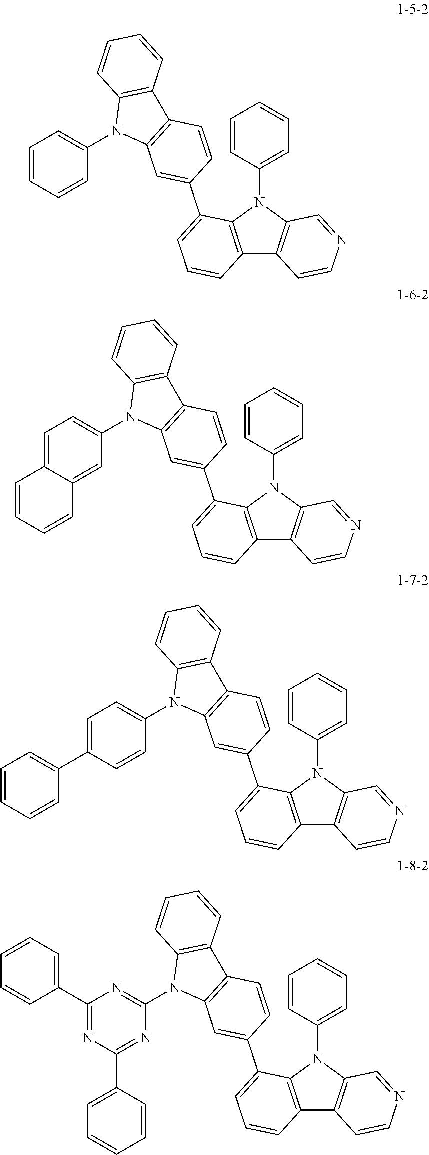 [SMILES: c1ccc(-n2c3ccccc3c3ccc(-c4cccc5c6ccncc6n(-c6ccccc6)c45)cc32)cc1 ; c1ccc(-n2c3cnccc3c3cccc(-c4ccc5c6ccccc6n(-c6ccc7ccccc7c6)c5c4)c32)cc1 ; c1ccc(-c2ccc(-n3c4ccccc4c4ccc(-c5cccc6c7ccncc7n(-c7ccccc7)c56)cc43)cc2)cc1 ; c1ccc(-c2nc(-c3ccccc3)nc(-n3c4ccccc4c4ccc(-c5cccc6c7ccncc7n(-c7ccccc7)c56)cc43)n2)cc1]

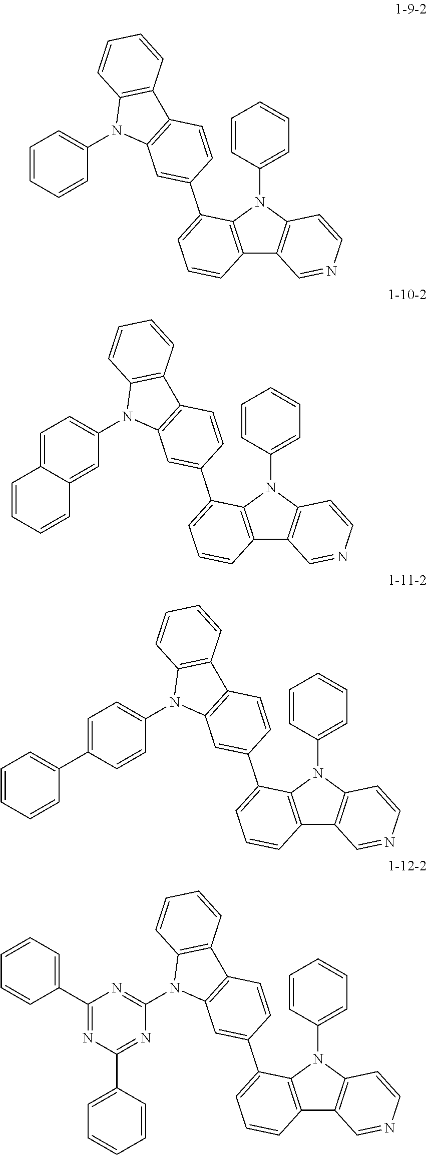 [SMILES: c1ccc(-n2c3ccccc3c3ccc(-c4cccc5c6cnccc6n(-c6ccccc6)c45)cc32)cc1 ; c1ccc(-n2c3ccncc3c3cccc(-c4ccc5c6ccccc6n(-c6ccc7ccccc7c6)c5c4)c32)cc1 ; c1ccc(-c2ccc(-n3c4ccccc4c4ccc(-c5cccc6c7cnccc7n(-c7ccccc7)c56)cc43)cc2)cc1 ; c1ccc(-c2nc(-c3ccccc3)nc(-n3c4ccccc4c4ccc(-c5cccc6c7cnccc7n(-c7ccccc7)c56)cc43)n2)cc1]

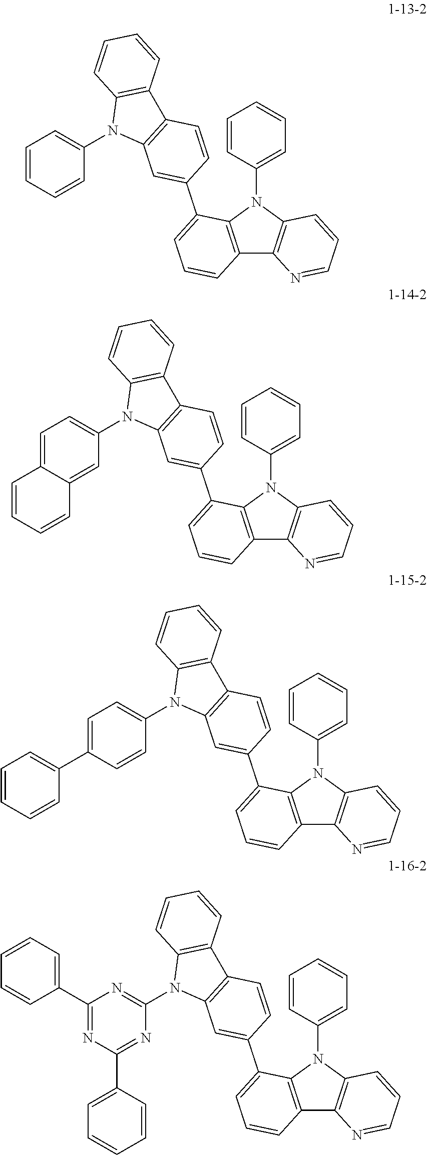 [SMILES: c1ccc(-n2c3ccccc3c3ccc(-c4cccc5c6ncccc6n(-c6ccccc6)c45)cc32)cc1 ; c1ccc(-n2c3cccnc3c3cccc(-c4ccc5c6ccccc6n(-c6ccc7ccccc7c6)c5c4)c32)cc1 ; c1ccc(-c2ccc(-n3c4ccccc4c4ccc(-c5cccc6c7ncccc7n(-c7ccccc7)c56)cc43)cc2)cc1 ; c1ccc(-c2nc(-c3ccccc3)nc(-n3c4ccccc4c4ccc(-c5cccc6c7ncccc7n(-c7ccccc7)c56)cc43)n2)cc1]

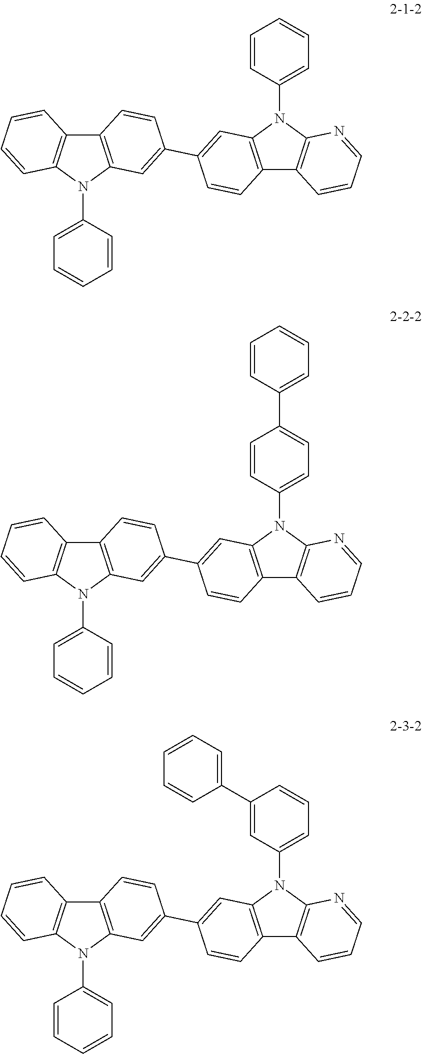 [SMILES: c1ccc(-n2c3ccccc3c3ccc(-c4ccc5c6cccnc6n(-c6ccccc6)c5c4)cc32)cc1 ; c1ccc(-c2ccc(-n3c4cc(-c5ccc6c7ccccc7n(-c7ccccc7)c6c5)ccc4c4cccnc43)cc2)cc1 ; c1ccc(-c2cccc(-n3c4cc(-c5ccc6c7ccccc7n(-c7ccccc7)c6c5)ccc4c4cccnc43)c2)cc1]

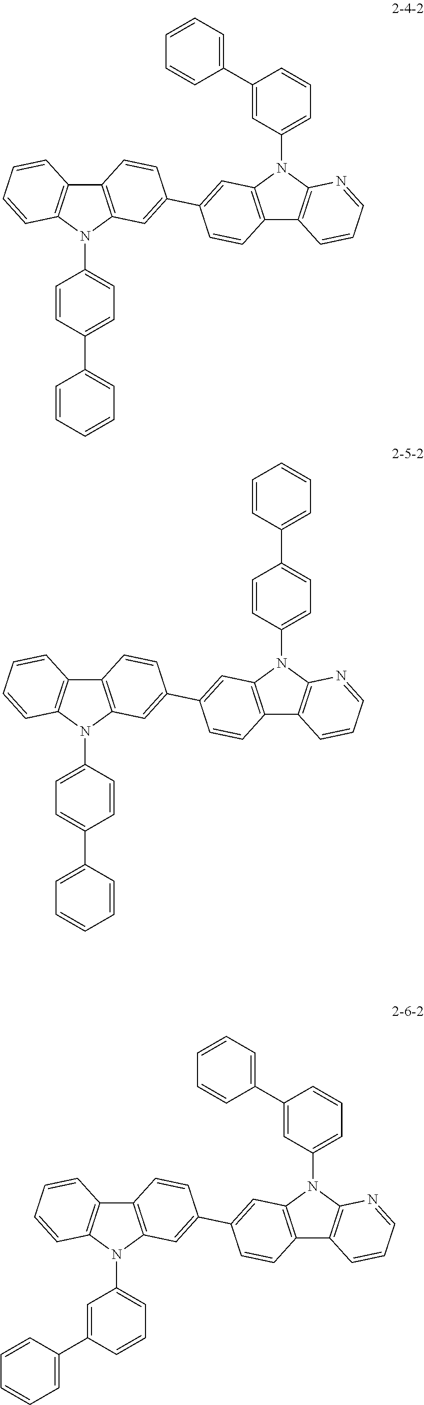 [SMILES: c1ccc(-c2ccc(-n3c4ccccc4c4ccc(-c5ccc6c7cccnc7n(-c7cccc(-c8ccccc8)c7)c6c5)cc43)cc2)cc1 ; c1ccc(-c2ccc(-n3c4ccccc4c4ccc(-c5ccc6c7cccnc7n(-c7ccc(-c8ccccc8)cc7)c6c5)cc43)cc2)cc1 ; c1ccc(-c2cccc(-n3c4ccccc4c4ccc(-c5ccc6c7cccnc7n(-c7cccc(-c8ccccc8)c7)c6c5)cc43)c2)cc1]

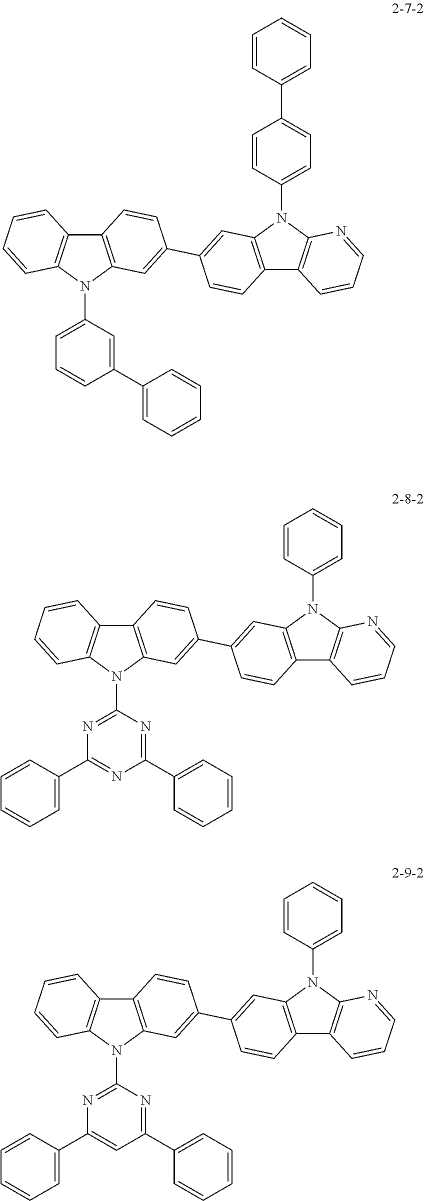 [SMILES: c1ccc(-c2ccc(-n3c4cc(-c5ccc6c7ccccc7n(-c7cccc(-c8ccccc8)c7)c6c5)ccc4c4cccnc43)cc2)cc1 ; c1ccc(-c2nc(-c3ccccc3)nc(-n3c4ccccc4c4ccc(-c5ccc6c7cccnc7n(-c7ccccc7)c6c5)cc43)n2)cc1 ; c1ccc(-c2cc(-c3ccccc3)nc(-n3c4ccccc4c4ccc(-c5ccc6c7cccnc7n(-c7ccccc7)c6c5)cc43)n2)cc1]

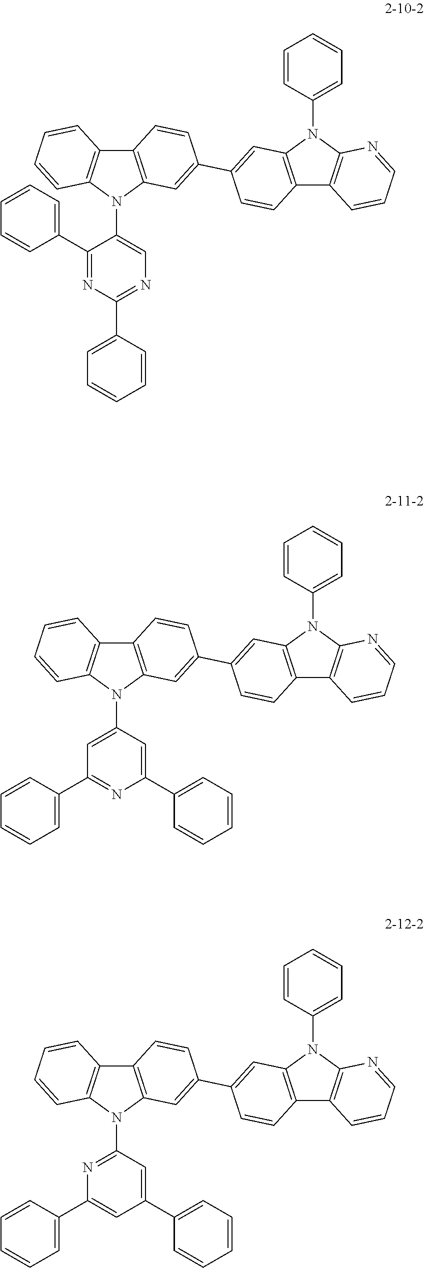 [SMILES: c1ccc(-c2ncc(-n3c4ccccc4c4ccc(-c5ccc6c7cccnc7n(-c7ccccc7)c6c5)cc43)c(-c3ccccc3)n2)cc1 ; c1ccc(-c2cc(-n3c4ccccc4c4ccc(-c5ccc6c7cccnc7n(-c7ccccc7)c6c5)cc43)cc(-c3ccccc3)n2)cc1 ; c1ccc(-c2cc(-c3ccccc3)nc(-n3c4ccccc4c4ccc(-c5ccc6c7cccnc7n(-c7ccccc7)c6c5)cc43)c2)cc1]

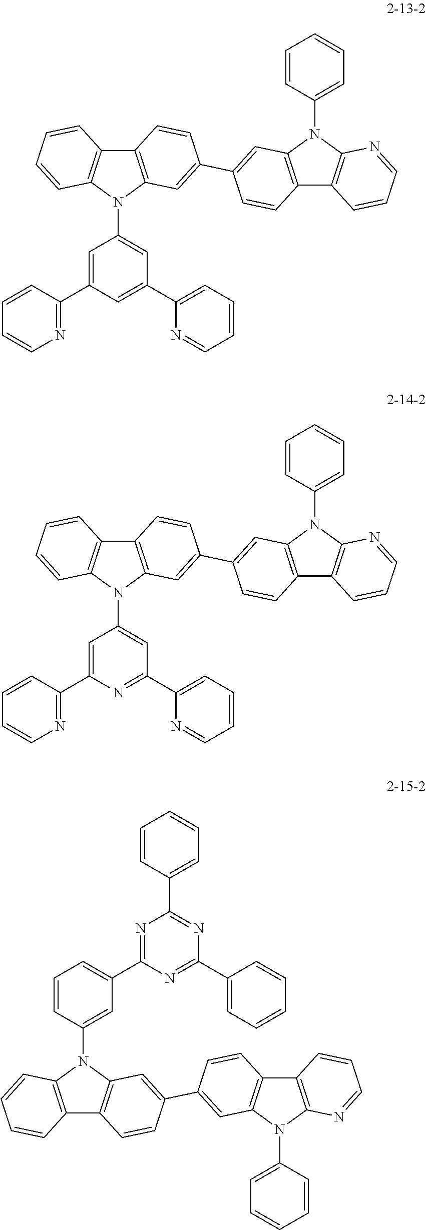 [SMILES: c1ccc(-n2c3cc(-c4ccc5c6ccccc6n(-c6cc(-c7ccccn7)cc(-c7ccccn7)c6)c5c4)ccc3c3cccnc32)cc1 ; c1ccc(-n2c3cc(-c4ccc5c6ccccc6n(-c6cc(-c7ccccn7)nc(-c7ccccn7)c6)c5c4)ccc3c3cccnc32)cc1 ; c1ccc(-c2nc(-c3ccccc3)nc(-c3cccc(-n4c5ccccc5c5ccc(-c6ccc7c8cccnc8n(-c8ccccc8)c7c6)cc54)c3)n2)cc1]

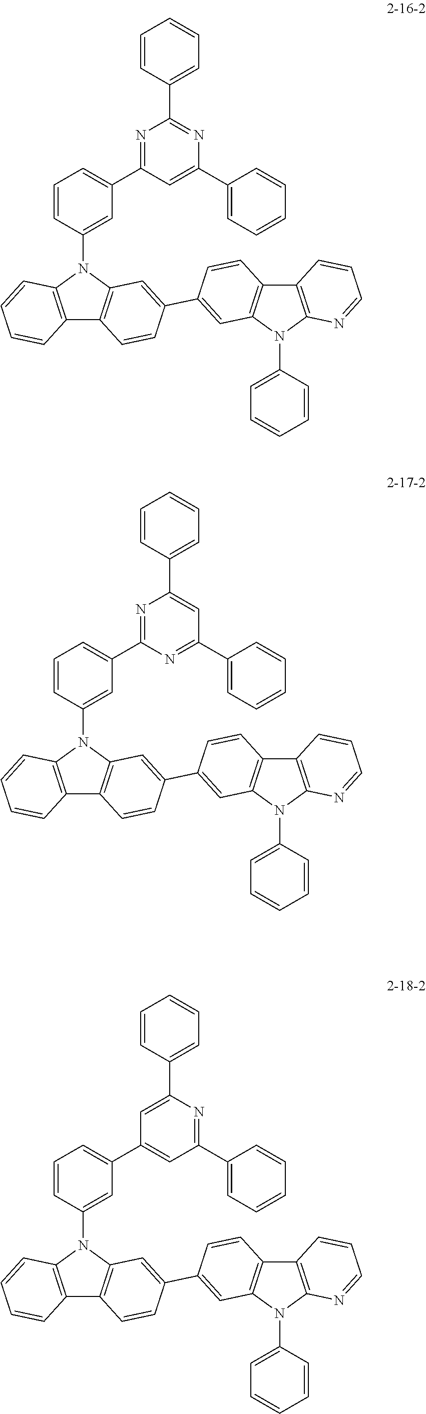 [SMILES: c1ccc(-c2cc(-c3cccc(-n4c5ccccc5c5ccc(-c6ccc7c8cccnc8n(-c8ccccc8)c7c6)cc54)c3)nc(-c3ccccc3)n2)cc1 ; c1ccc(-c2cc(-c3ccccc3)nc(-c3cccc(-n4c5ccccc5c5ccc(-c6ccc7c8cccnc8n(-c8ccccc8)c7c6)cc54)c3)n2)cc1 ; c1ccc(-c2cc(-c3cccc(-n4c5ccccc5c5ccc(-c6ccc7c8cccnc8n(-c8ccccc8)c7c6)cc54)c3)cc(-c3ccccc3)n2)cc1]

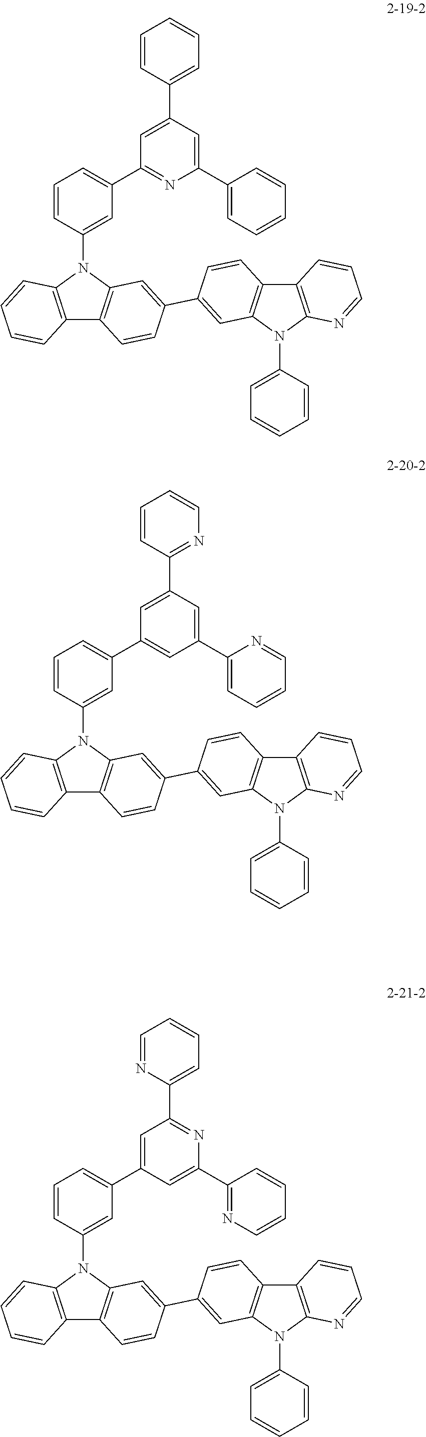 [SMILES: c1ccc(-c2cc(-c3ccccc3)nc(-c3cccc(-n4c5ccccc5c5ccc(-c6ccc7c8cccnc8n(-c8ccccc8)c7c6)cc54)c3)c2)cc1 ; c1ccc(-n2c3cc(-c4ccc5c6ccccc6n(-c6cccc(-c7cc(-c8ccccn8)cc(-c8ccccn8)c7)c6)c5c4)ccc3c3cccnc32)cc1 ; c1ccc(-n2c3cc(-c4ccc5c6ccccc6n(-c6cccc(-c7cc(-c8ccccn8)nc(-c8ccccn8)c7)c6)c5c4)ccc3c3cccnc32)cc1]

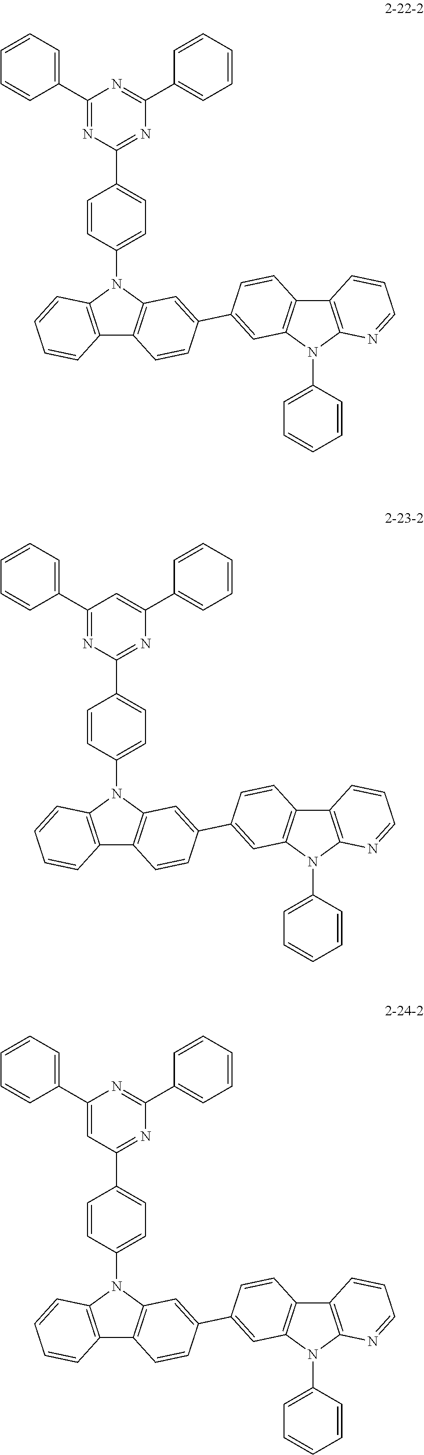 [SMILES: c1ccc(-c2nc(-c3ccccc3)nc(-c3ccc(-n4c5ccccc5c5ccc(-c6ccc7c8cccnc8n(-c8ccccc8)c7c6)cc54)cc3)n2)cc1 ; c1ccc(-c2cc(-c3ccccc3)nc(-c3ccc(-n4c5ccccc5c5ccc(-c6ccc7c8cccnc8n(-c8ccccc8)c7c6)cc54)cc3)n2)cc1 ; c1ccc(-c2cc(-c3ccc(-n4c5ccccc5c5ccc(-c6ccc7c8cccnc8n(-c8ccccc8)c7c6)cc54)cc3)nc(-c3ccccc3)n2)cc1]

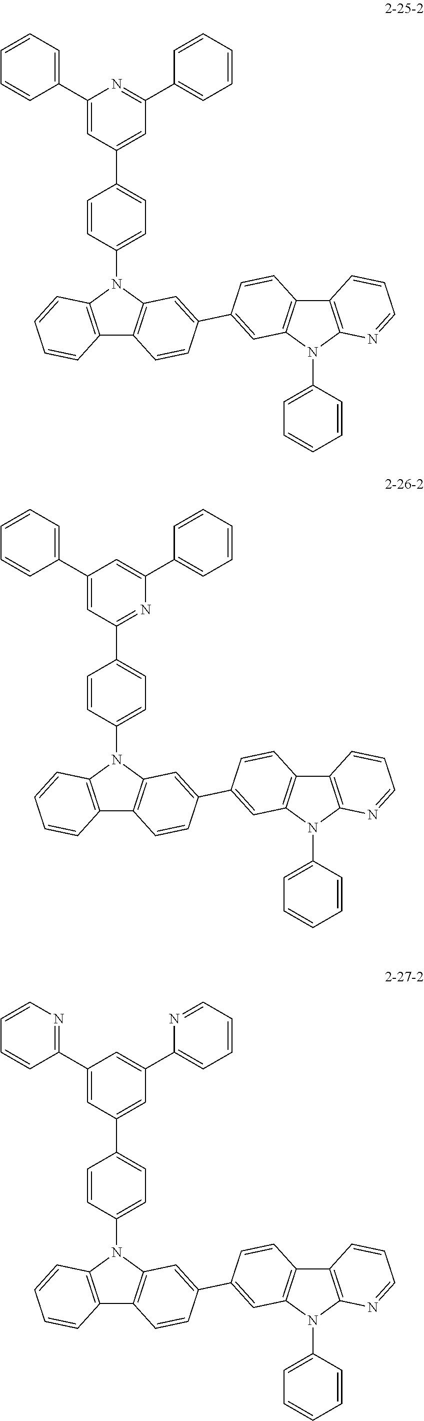 [SMILES: c1ccc(-c2cc(-c3ccc(-n4c5ccccc5c5ccc(-c6ccc7c8cccnc8n(-c8ccccc8)c7c6)cc54)cc3)cc(-c3ccccc3)n2)cc1 ; c1ccc(-c2cc(-c3ccccc3)nc(-c3ccc(-n4c5ccccc5c5ccc(-c6ccc7c8cccnc8n(-c8ccccc8)c7c6)cc54)cc3)c2)cc1 ; c1ccc(-n2c3cc(-c4ccc5c6ccccc6n(-c6ccc(-c7cc(-c8ccccn8)cc(-c8ccccn8)c7)cc6)c5c4)ccc3c3cccnc32)cc1]

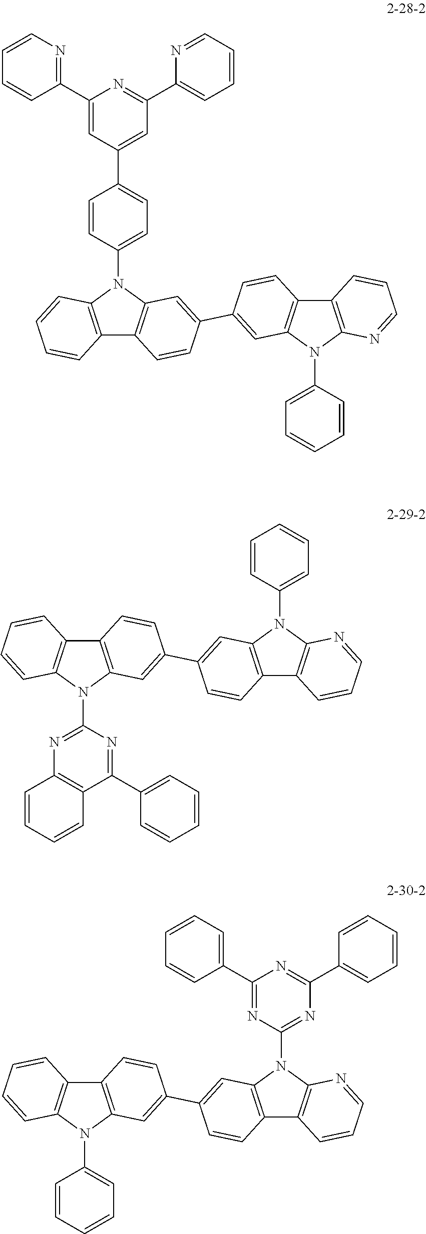 [SMILES: c1ccc(-n2c3cc(-c4ccc5c6ccccc6n(-c6ccc(-c7cc(-c8ccccn8)nc(-c8ccccn8)c7)cc6)c5c4)ccc3c3cccnc32)cc1 ; c1ccc(-c2nc(-n3c4ccccc4c4ccc(-c5ccc6c7cccnc7n(-c7ccccc7)c6c5)cc43)nc3ccccc23)cc1 ; c1ccc(-c2nc(-c3ccccc3)nc(-n3c4cc(-c5ccc6c7ccccc7n(-c7ccccc7)c6c5)ccc4c4cccnc43)n2)cc1]

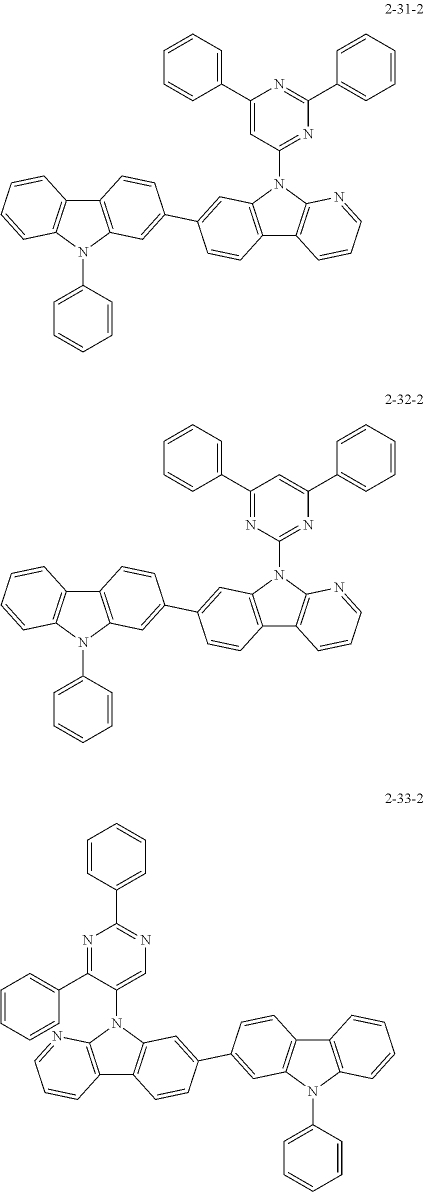 [SMILES: c1ccc(-c2cc(-n3c4cc(-c5ccc6c7ccccc7n(-c7ccccc7)c6c5)ccc4c4cccnc43)nc(-c3ccccc3)n2)cc1 ; c1ccc(-c2cc(-c3ccccc3)nc(-n3c4cc(-c5ccc6c7ccccc7n(-c7ccccc7)c6c5)ccc4c4cccnc43)n2)cc1 ; c1ccc(-c2ncc(-n3c4cc(-c5ccc6c7ccccc7n(-c7ccccc7)c6c5)ccc4c4cccnc43)c(-c3ccccc3)n2)cc1]

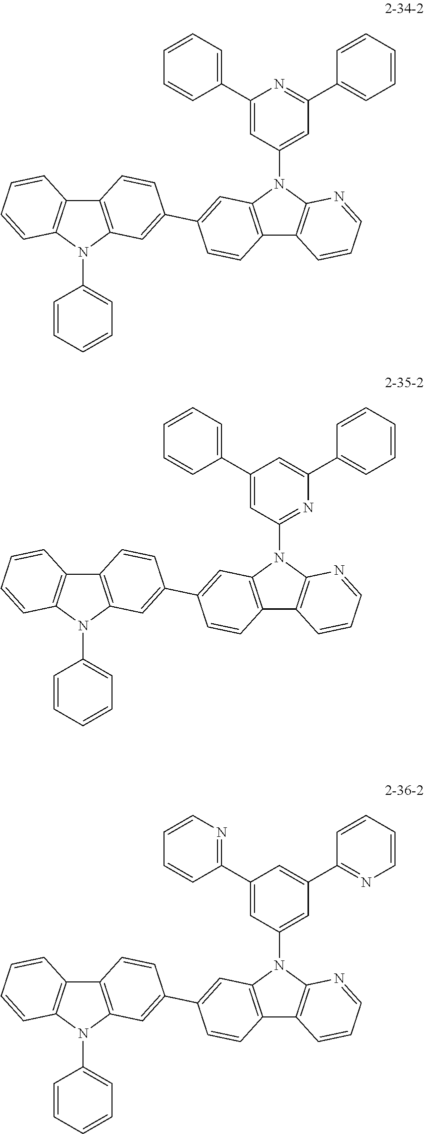 [SMILES: c1ccc(-c2cc(-n3c4cc(-c5ccc6c7ccccc7n(-c7ccccc7)c6c5)ccc4c4cccnc43)cc(-c3ccccc3)n2)cc1 ; c1ccc(-c2cc(-c3ccccc3)nc(-n3c4cc(-c5ccc6c7ccccc7n(-c7ccccc7)c6c5)ccc4c4cccnc43)c2)cc1 ; c1ccc(-n2c3ccccc3c3ccc(-c4ccc5c6cccnc6n(-c6cc(-c7ccccn7)cc(-c7ccccn7)c6)c5c4)cc32)cc1]

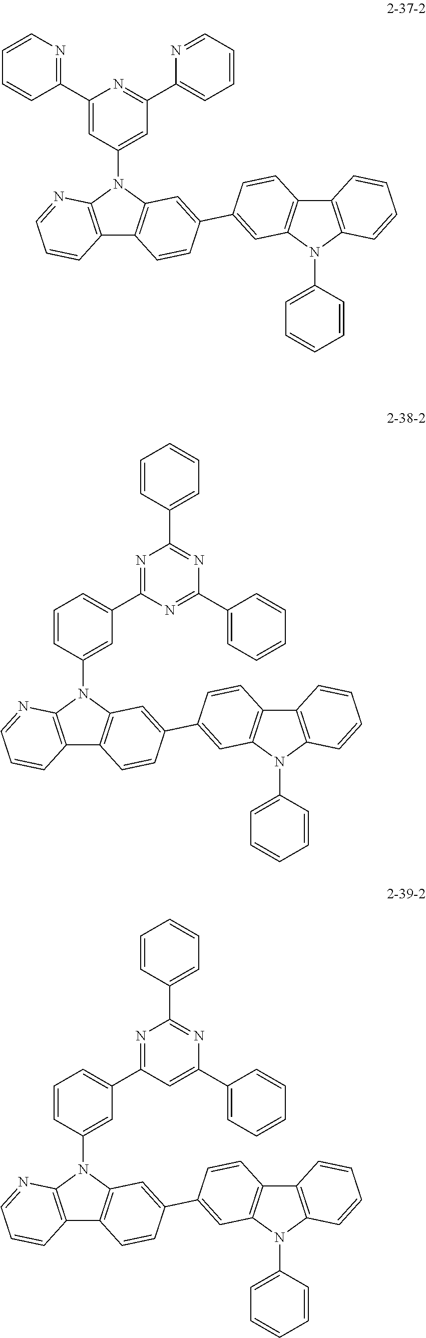 [SMILES: c1ccc(-n2c3ccccc3c3ccc(-c4ccc5c6cccnc6n(-c6cc(-c7ccccn7)nc(-c7ccccn7)c6)c5c4)cc32)cc1 ; c1ccc(-c2nc(-c3ccccc3)nc(-c3cccc(-n4c5cc(-c6ccc7c8ccccc8n(-c8ccccc8)c7c6)ccc5c5cccnc54)c3)n2)cc1 ; c1ccc(-c2cc(-c3cccc(-n4c5cc(-c6ccc7c8ccccc8n(-c8ccccc8)c7c6)ccc5c5cccnc54)c3)nc(-c3ccccc3)n2)cc1]

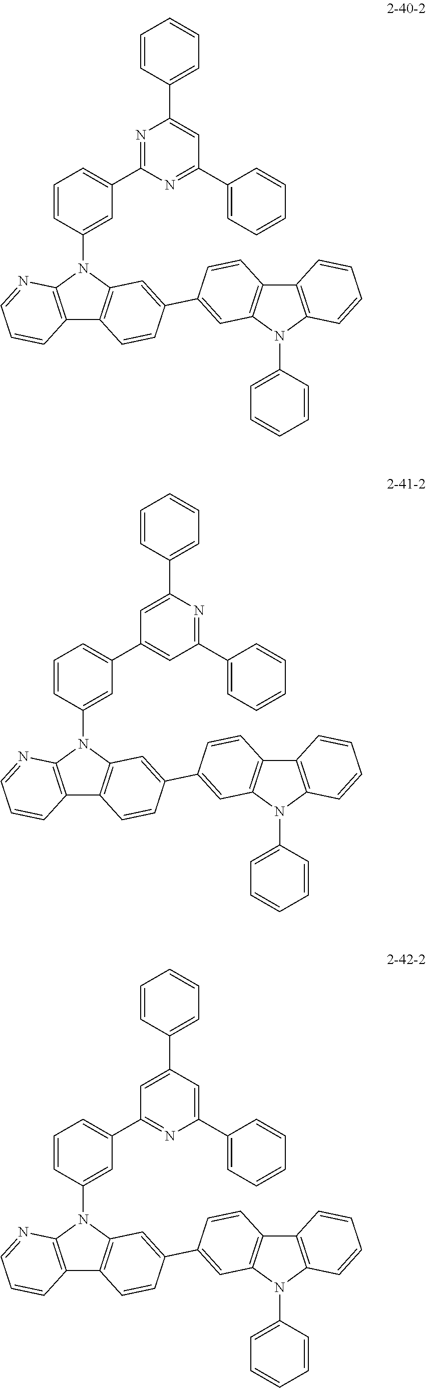 [SMILES: c1ccc(-c2cc(-c3ccccc3)nc(-c3cccc(-n4c5cc(-c6ccc7c8ccccc8n(-c8ccccc8)c7c6)ccc5c5cccnc54)c3)n2)cc1 ; c1ccc(-c2cc(-c3cccc(-n4c5cc(-c6ccc7c8ccccc8n(-c8ccccc8)c7c6)ccc5c5cccnc54)c3)cc(-c3ccccc3)n2)cc1 ; c1ccc(-c2cc(-c3ccccc3)nc(-c3cccc(-n4c5cc(-c6ccc7c8ccccc8n(-c8ccccc8)c7c6)ccc5c5cccnc54)c3)c2)cc1]

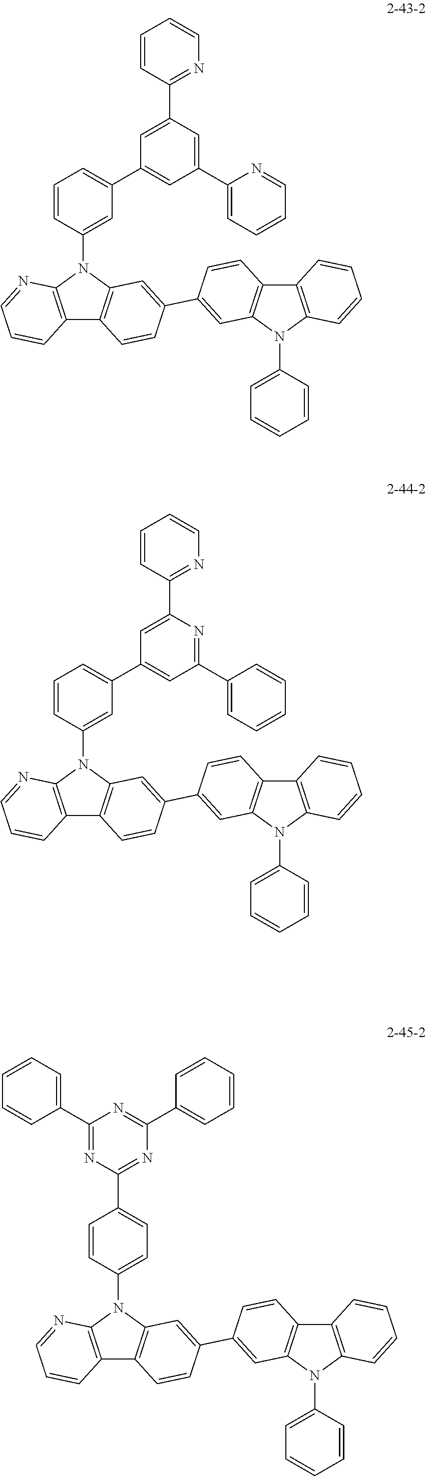 [SMILES: c1ccc(-n2c3ccccc3c3ccc(-c4ccc5c6cccnc6n(-c6cccc(-c7cc(-c8ccccn8)cc(-c8ccccn8)c7)c6)c5c4)cc32)cc1 ; c1ccc(-c2cc(-c3cccc(-n4c5cc(-c6ccc7c8ccccc8n(-c8ccccc8)c7c6)ccc5c5cccnc54)c3)cc(-c3ccccn3)n2)cc1 ; c1ccc(-c2nc(-c3ccccc3)nc(-c3ccc(-n4c5cc(-c6ccc7c8ccccc8n(-c8ccccc8)c7c6)ccc5c5cccnc54)cc3)n2)cc1]

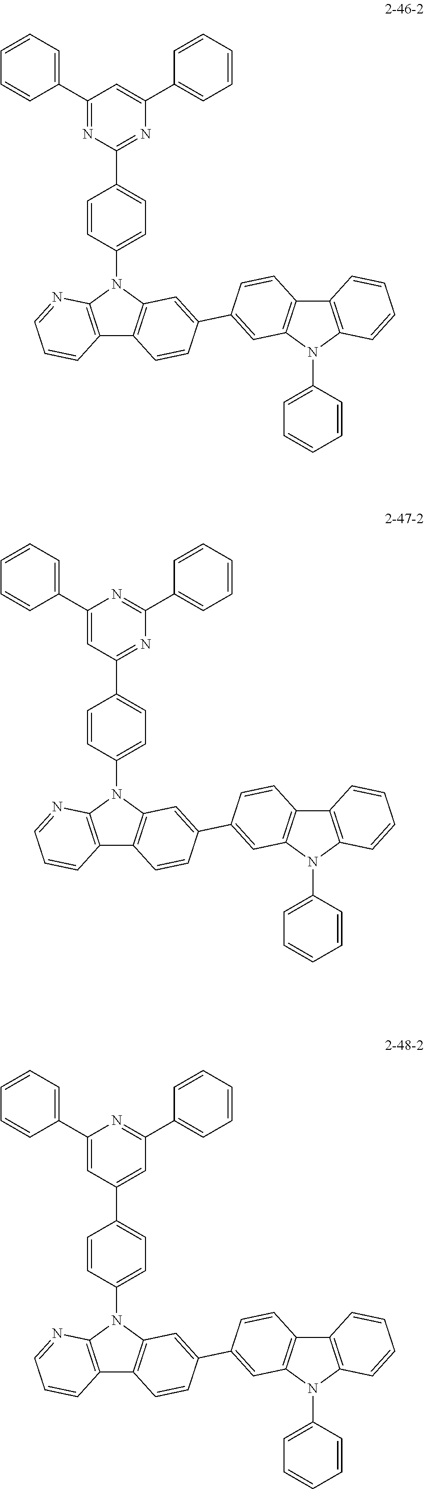 [SMILES: c1ccc(-c2cc(-c3ccccc3)nc(-c3ccc(-n4c5cc(-c6ccc7c8ccccc8n(-c8ccccc8)c7c6)ccc5c5cccnc54)cc3)n2)cc1 ; c1ccc(-c2cc(-c3ccc(-n4c5cc(-c6ccc7c8ccccc8n(-c8ccccc8)c7c6)ccc5c5cccnc54)cc3)nc(-c3ccccc3)n2)cc1 ; c1ccc(-c2cc(-c3ccc(-n4c5cc(-c6ccc7c8ccccc8n(-c8ccccc8)c7c6)ccc5c5cccnc54)cc3)cc(-c3ccccc3)n2)cc1]

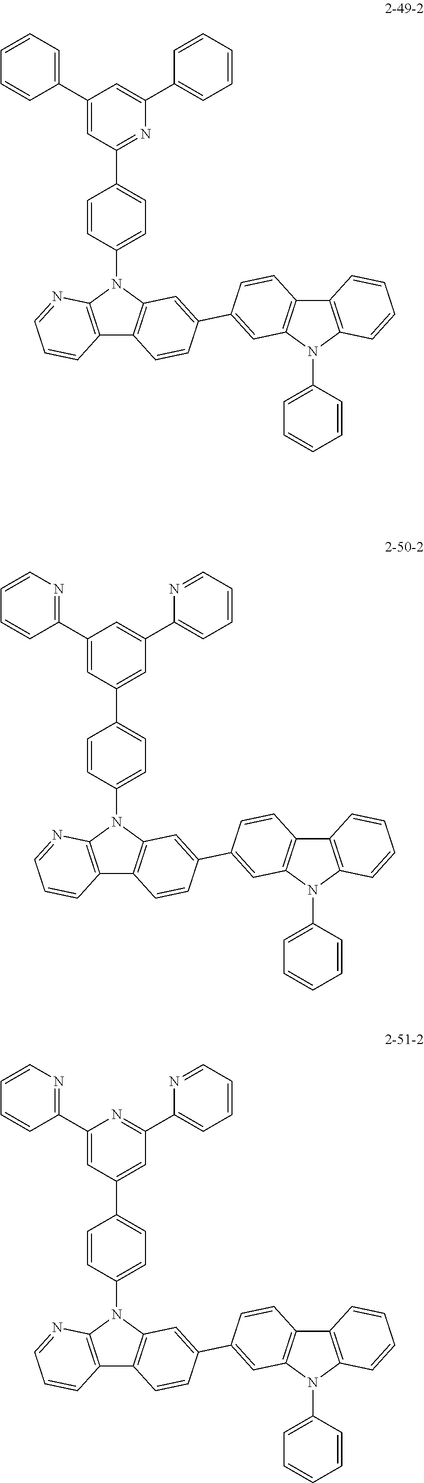 [SMILES: c1ccc(-c2cc(-c3ccccc3)nc(-c3ccc(-n4c5cc(-c6ccc7c8ccccc8n(-c8ccccc8)c7c6)ccc5c5cccnc54)cc3)c2)cc1 ; c1ccc(-n2c3ccccc3c3ccc(-c4ccc5c6cccnc6n(-c6ccc(-c7cc(-c8ccccn8)cc(-c8ccccn8)c7)cc6)c5c4)cc32)cc1 ; c1ccc(-n2c3ccccc3c3ccc(-c4ccc5c6cccnc6n(-c6ccc(-c7cc(-c8ccccn8)nc(-c8ccccn8)c7)cc6)c5c4)cc32)cc1]

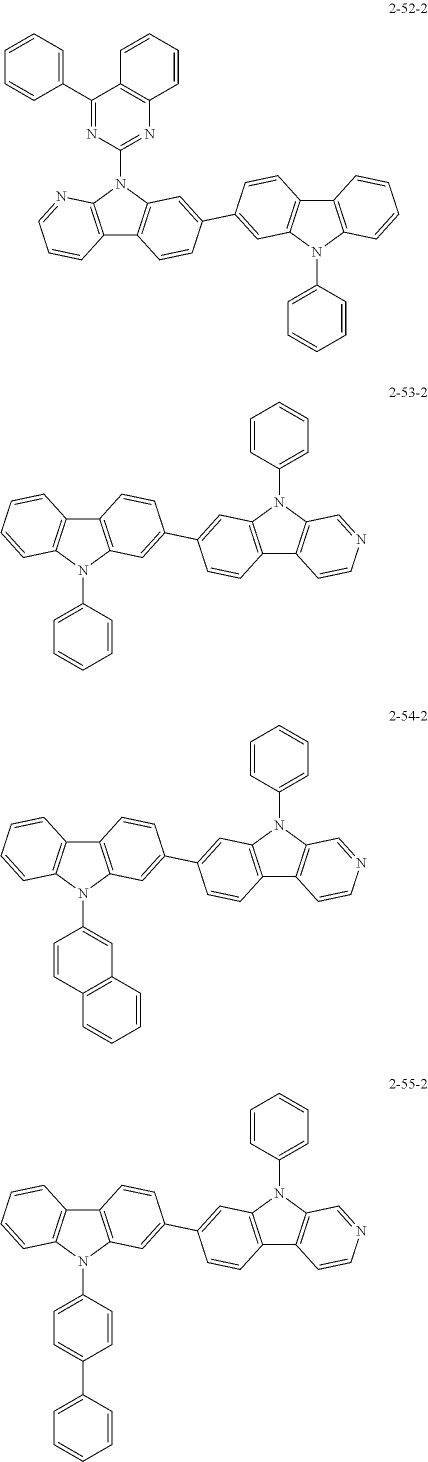 [SMILES: c1ccc(-c2nc(-n3c4cc(-c5ccc6c7ccccc7n(-c7ccccc7)c6c5)ccc4c4cccnc43)nc3ccccc23)cc1 ; c1ccc(-n2c3ccccc3c3ccc(-c4ccc5c6ccncc6n(-c6ccccc6)c5c4)cc32)cc1 ; c1ccc(-n2c3cnccc3c3ccc(-c4ccc5c6ccccc6n(-c6ccc7ccccc7c6)c5c4)cc32)cc1 ; c1ccc(-c2ccc(-n3c4ccccc4c4ccc(-c5ccc6c7ccncc7n(-c7ccccc7)c6c5)cc43)cc2)cc1]

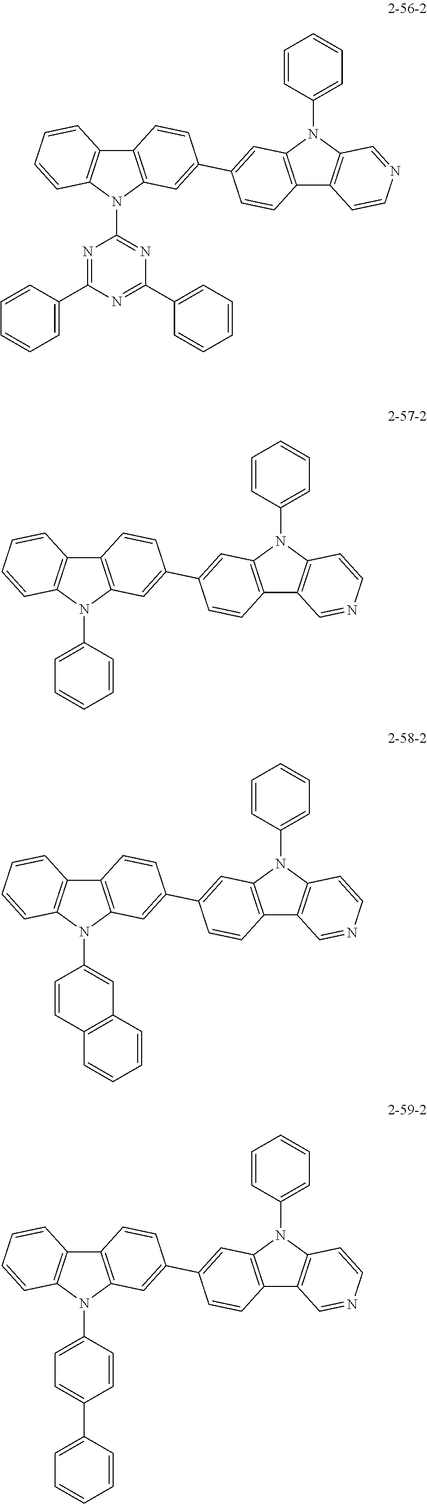 [SMILES: c1ccc(-c2nc(-c3ccccc3)nc(-n3c4ccccc4c4ccc(-c5ccc6c7ccncc7n(-c7ccccc7)c6c5)cc43)n2)cc1 ; c1ccc(-n2c3ccccc3c3ccc(-c4ccc5c6cnccc6n(-c6ccccc6)c5c4)cc32)cc1 ; c1ccc(-n2c3ccncc3c3ccc(-c4ccc5c6ccccc6n(-c6ccc7ccccc7c6)c5c4)cc32)cc1 ; c1ccc(-c2ccc(-n3c4ccccc4c4ccc(-c5ccc6c7cnccc7n(-c7ccccc7)c6c5)cc43)cc2)cc1]

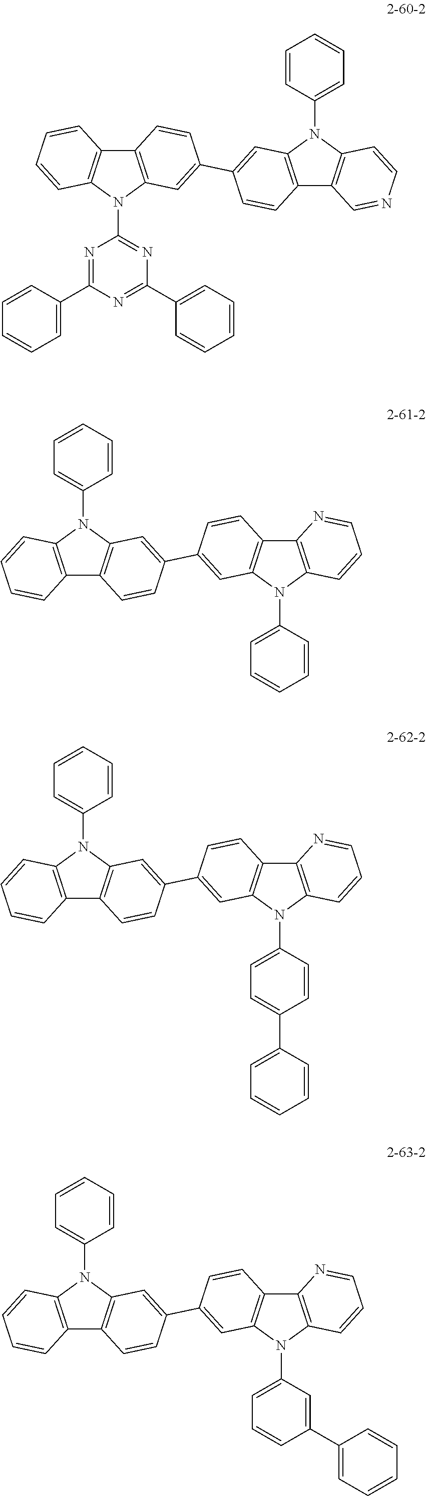 [SMILES: c1ccc(-c2nc(-c3ccccc3)nc(-n3c4ccccc4c4ccc(-c5ccc6c7cnccc7n(-c7ccccc7)c6c5)cc43)n2)cc1 ; c1ccc(-n2c3ccccc3c3ccc(-c4ccc5c6ncccc6n(-c6ccccc6)c5c4)cc32)cc1 ; c1ccc(-c2ccc(-n3c4cc(-c5ccc6c7ccccc7n(-c7ccccc7)c6c5)ccc4c4ncccc43)cc2)cc1 ; c1ccc(-c2cccc(-n3c4cc(-c5ccc6c7ccccc7n(-c7ccccc7)c6c5)ccc4c4ncccc43)c2)cc1]

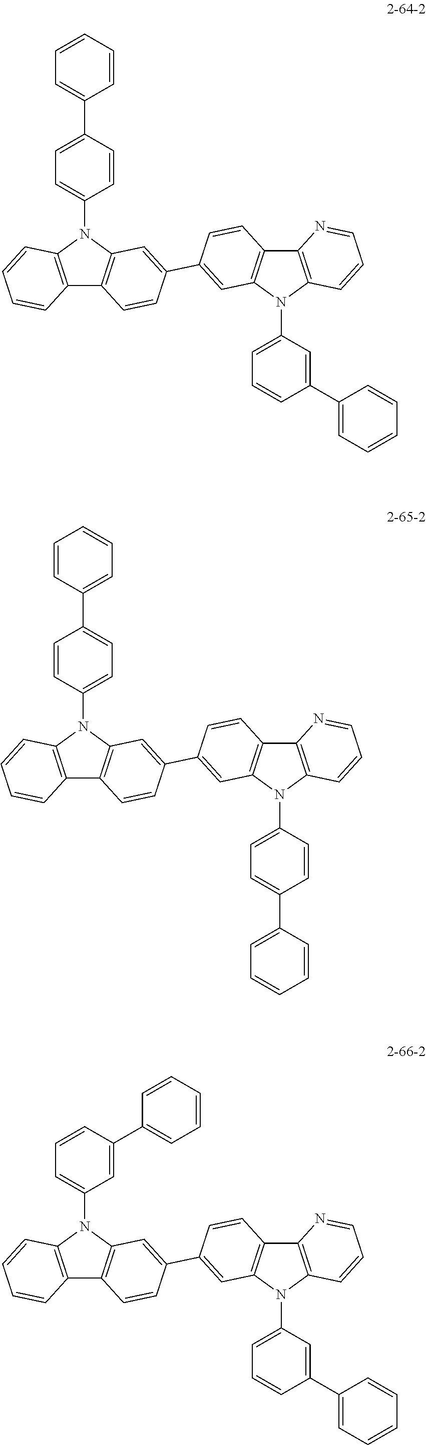 [SMILES: c1ccc(-c2ccc(-n3c4ccccc4c4ccc(-c5ccc6c7ncccc7n(-c7cccc(-c8ccccc8)c7)c6c5)cc43)cc2)cc1 ; c1ccc(-c2ccc(-n3c4ccccc4c4ccc(-c5ccc6c7ncccc7n(-c7ccc(-c8ccccc8)cc7)c6c5)cc43)cc2)cc1 ; c1ccc(-c2cccc(-n3c4ccccc4c4ccc(-c5ccc6c7ncccc7n(-c7cccc(-c8ccccc8)c7)c6c5)cc43)c2)cc1]

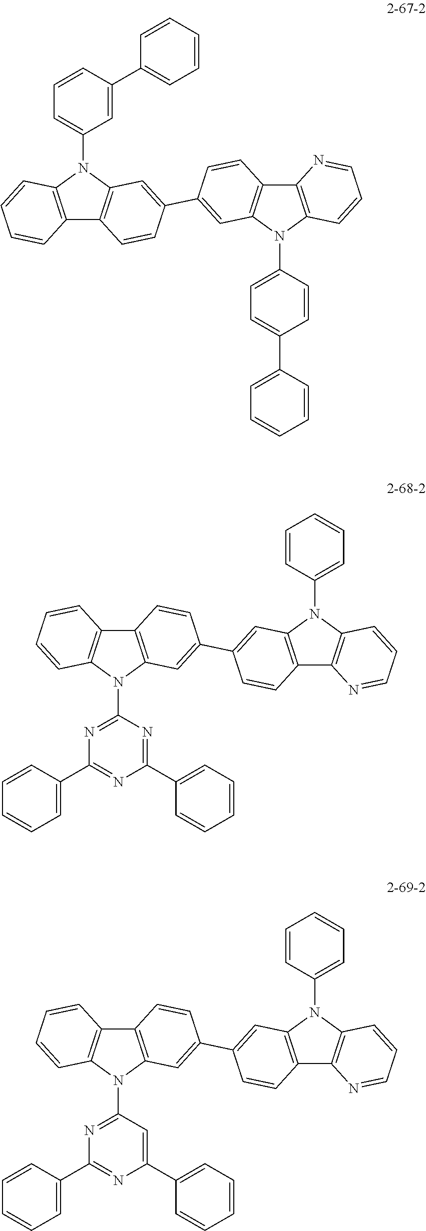 [SMILES: c1ccc(-c2ccc(-n3c4cc(-c5ccc6c7ccccc7n(-c7cccc(-c8ccccc8)c7)c6c5)ccc4c4ncccc43)cc2)cc1 ; c1ccc(-c2nc(-c3ccccc3)nc(-n3c4ccccc4c4ccc(-c5ccc6c7ncccc7n(-c7ccccc7)c6c5)cc43)n2)cc1 ; c1ccc(-c2cc(-n3c4ccccc4c4ccc(-c5ccc6c7ncccc7n(-c7ccccc7)c6c5)cc43)nc(-c3ccccc3)n2)cc1]

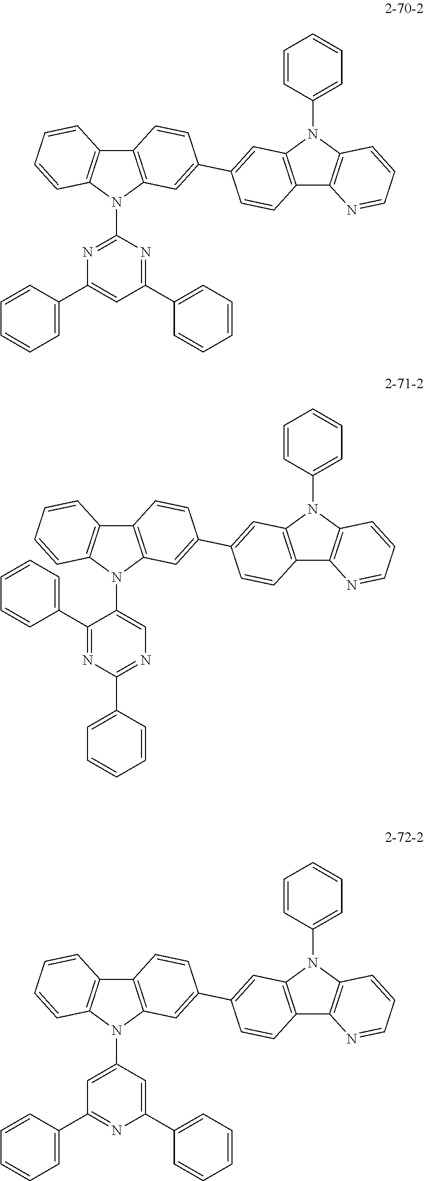 [SMILES: c1ccc(-c2cc(-c3ccccc3)nc(-n3c4ccccc4c4ccc(-c5ccc6c7ncccc7n(-c7ccccc7)c6c5)cc43)n2)cc1 ; c1ccc(-c2ncc(-n3c4ccccc4c4ccc(-c5ccc6c7ncccc7n(-c7ccccc7)c6c5)cc43)c(-c3ccccc3)n2)cc1 ; c1ccc(-c2cc(-n3c4ccccc4c4ccc(-c5ccc6c7ncccc7n(-c7ccccc7)c6c5)cc43)cc(-c3ccccc3)n2)cc1]

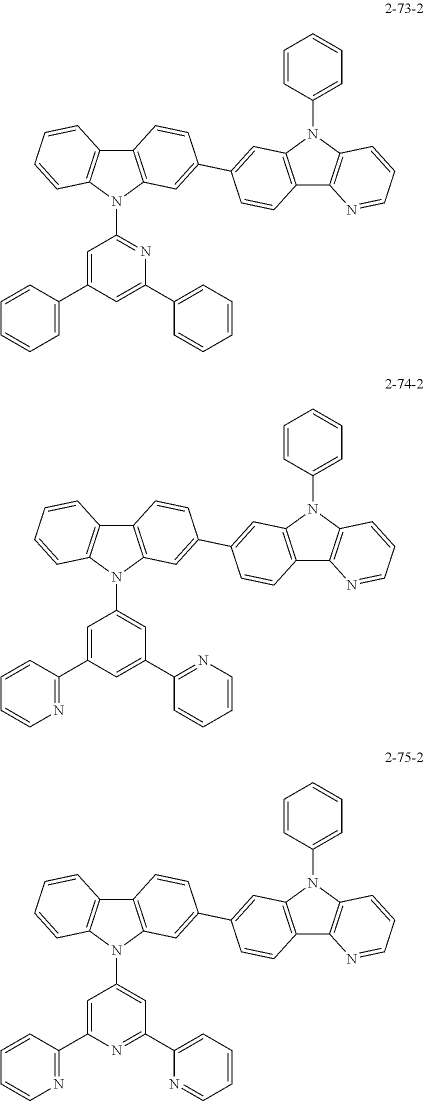 [SMILES: c1ccc(-c2cc(-c3ccccc3)nc(-n3c4ccccc4c4ccc(-c5ccc6c7ncccc7n(-c7ccccc7)c6c5)cc43)c2)cc1 ; c1ccc(-n2c3cc(-c4ccc5c6ccccc6n(-c6cc(-c7ccccn7)cc(-c7ccccn7)c6)c5c4)ccc3c3ncccc32)cc1 ; c1ccc(-n2c3cc(-c4ccc5c6ccccc6n(-c6cc(-c7ccccn7)nc(-c7ccccn7)c6)c5c4)ccc3c3ncccc32)cc1]

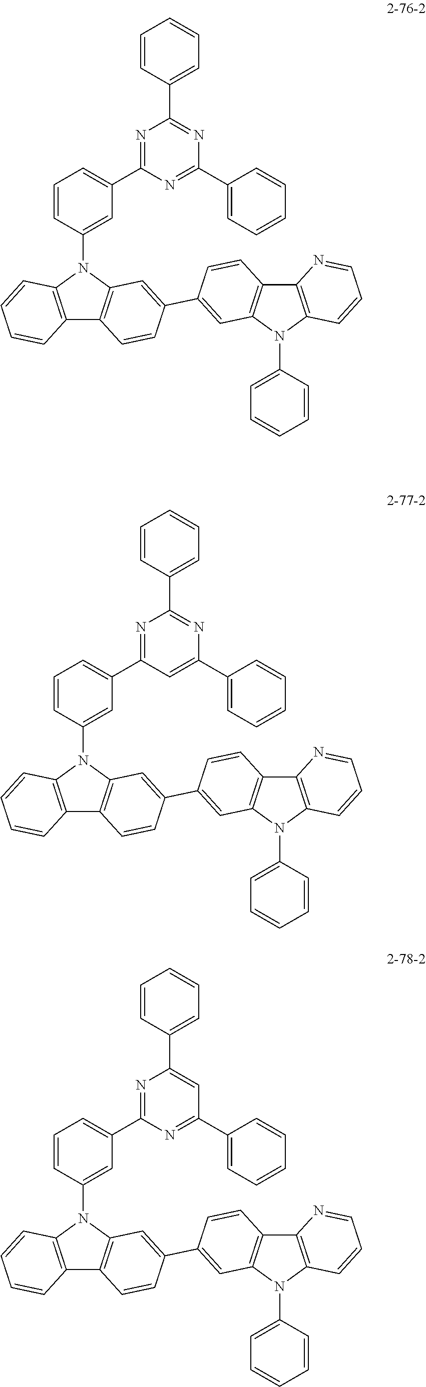 [SMILES: c1ccc(-c2nc(-c3ccccc3)nc(-c3cccc(-n4c5ccccc5c5ccc(-c6ccc7c8ncccc8n(-c8ccccc8)c7c6)cc54)c3)n2)cc1 ; c1ccc(-c2cc(-c3cccc(-n4c5ccccc5c5ccc(-c6ccc7c8ncccc8n(-c8ccccc8)c7c6)cc54)c3)nc(-c3ccccc3)n2)cc1 ; c1ccc(-c2cc(-c3ccccc3)nc(-c3cccc(-n4c5ccccc5c5ccc(-c6ccc7c8ncccc8n(-c8ccccc8)c7c6)cc54)c3)n2)cc1]

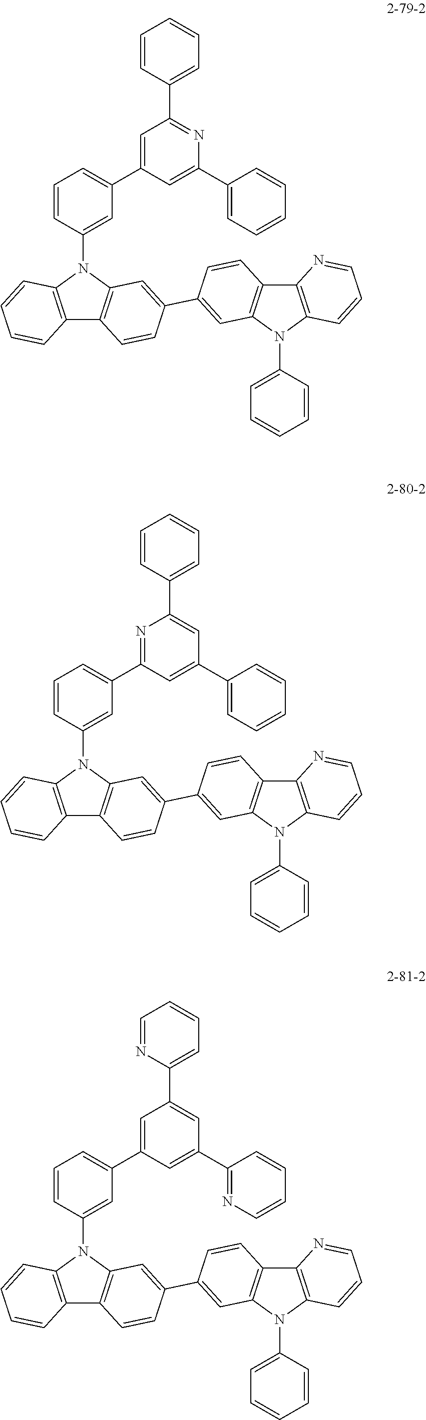 [SMILES: c1ccc(-c2cc(-c3cccc(-n4c5ccccc5c5ccc(-c6ccc7c8ncccc8n(-c8ccccc8)c7c6)cc54)c3)cc(-c3ccccc3)n2)cc1 ; c1ccc(-c2cc(-c3ccccc3)nc(-c3cccc(-n4c5ccccc5c5ccc(-c6ccc7c8ncccc8n(-c8ccccc8)c7c6)cc54)c3)c2)cc1 ; c1ccc(-n2c3cc(-c4ccc5c6ccccc6n(-c6cccc(-c7cc(-c8ccccn8)cc(-c8ccccn8)c7)c6)c5c4)ccc3c3ncccc32)cc1]

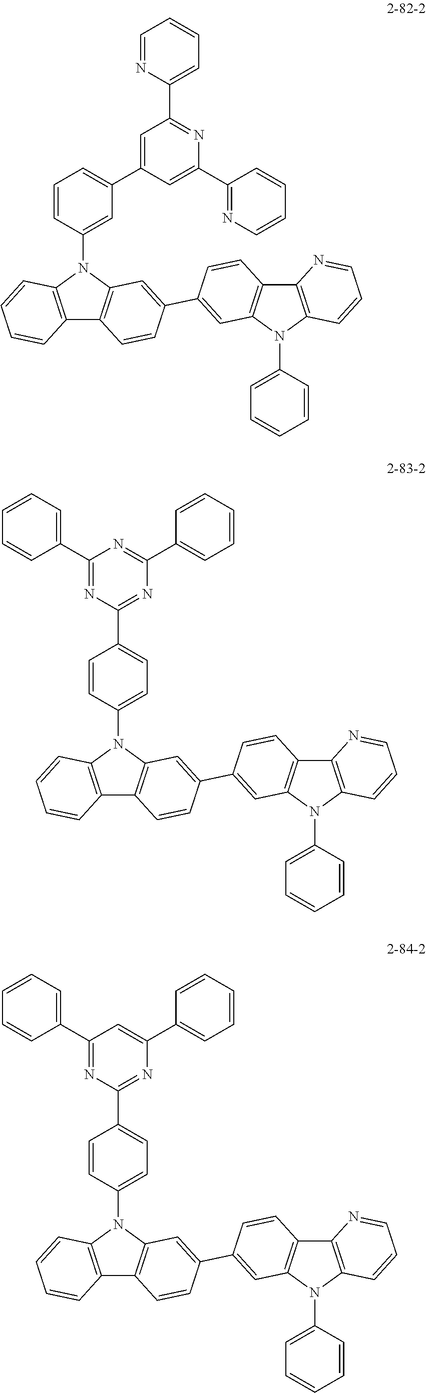 [SMILES: c1ccc(-n2c3cc(-c4ccc5c6ccccc6n(-c6cccc(-c7cc(-c8ccccn8)nc(-c8ccccn8)c7)c6)c5c4)ccc3c3ncccc32)cc1 ; c1ccc(-c2nc(-c3ccccc3)nc(-c3ccc(-n4c5ccccc5c5ccc(-c6ccc7c8ncccc8n(-c8ccccc8)c7c6)cc54)cc3)n2)cc1 ; c1ccc(-c2cc(-c3ccccc3)nc(-c3ccc(-n4c5ccccc5c5ccc(-c6ccc7c8ncccc8n(-c8ccccc8)c7c6)cc54)cc3)n2)cc1]

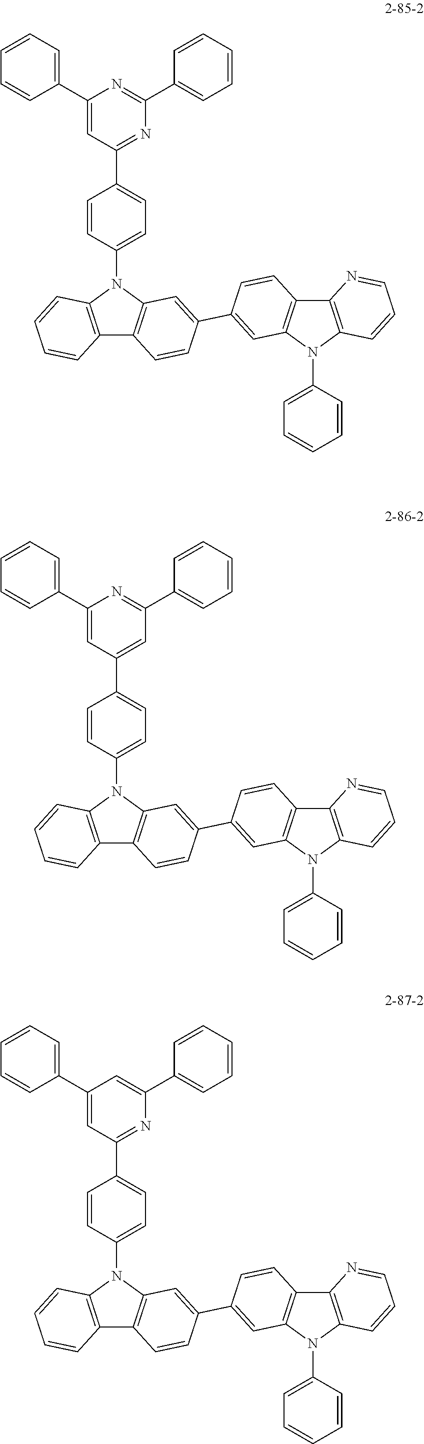 [SMILES: c1ccc(-c2cc(-c3ccc(-n4c5ccccc5c5ccc(-c6ccc7c8ncccc8n(-c8ccccc8)c7c6)cc54)cc3)nc(-c3ccccc3)n2)cc1 ; c1ccc(-c2cc(-c3ccc(-n4c5ccccc5c5ccc(-c6ccc7c8ncccc8n(-c8ccccc8)c7c6)cc54)cc3)cc(-c3ccccc3)n2)cc1 ; c1ccc(-c2cc(-c3ccccc3)nc(-c3ccc(-n4c5ccccc5c5ccc(-c6ccc7c8ncccc8n(-c8ccccc8)c7c6)cc54)cc3)c2)cc1]

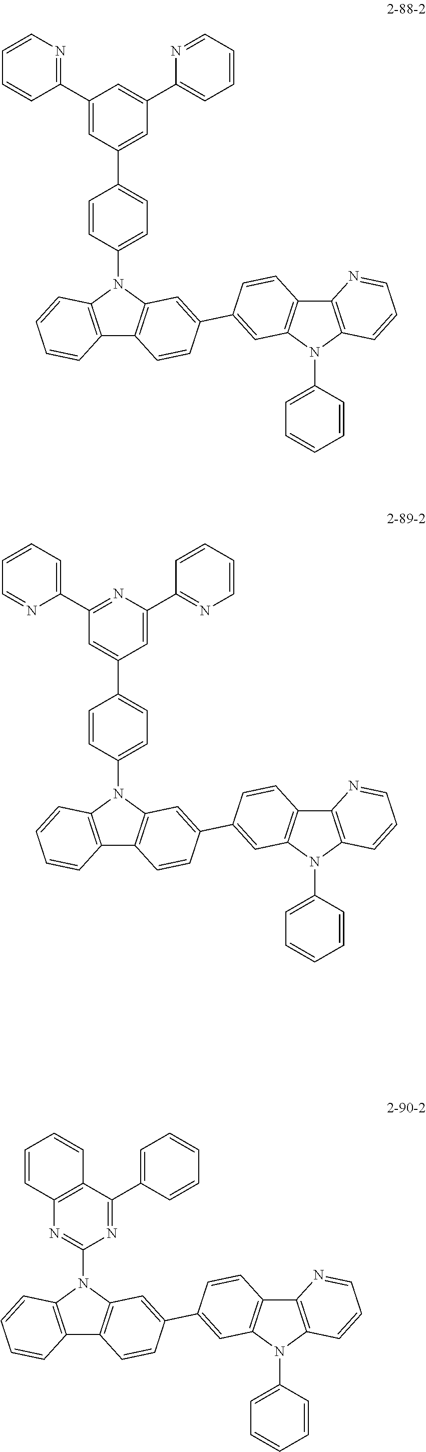 [SMILES: c1ccc(-n2c3cc(-c4ccc5c6ccccc6n(-c6ccc(-c7cc(-c8ccccn8)cc(-c8ccccn8)c7)cc6)c5c4)ccc3c3ncccc32)cc1 ; c1ccc(-n2c3cc(-c4ccc5c6ccccc6n(-c6ccc(-c7cc(-c8ccccn8)nc(-c8ccccn8)c7)cc6)c5c4)ccc3c3ncccc32)cc1 ; c1ccc(-c2nc(-n3c4ccccc4c4ccc(-c5ccc6c7ncccc7n(-c7ccccc7)c6c5)cc43)nc3ccccc23)cc1]

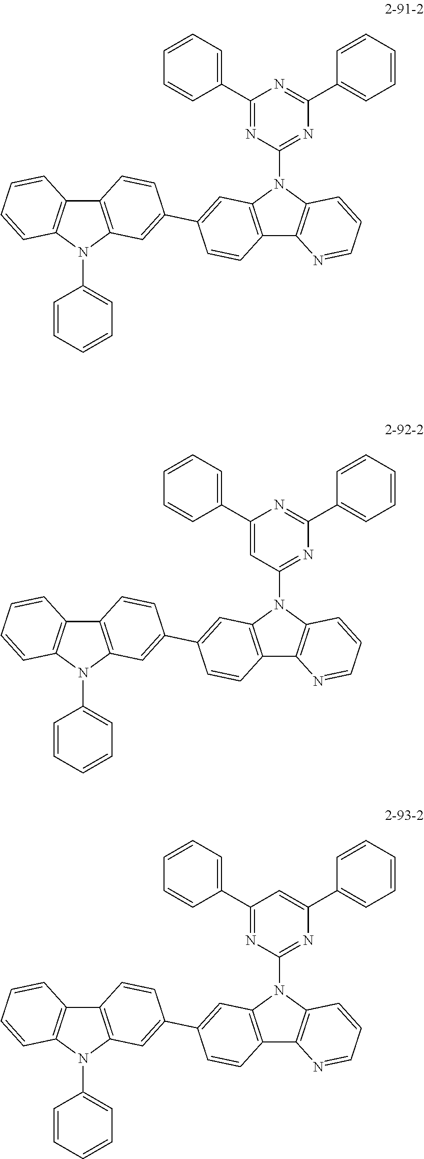 [SMILES: c1ccc(-c2nc(-c3ccccc3)nc(-n3c4cc(-c5ccc6c7ccccc7n(-c7ccccc7)c6c5)ccc4c4ncccc43)n2)cc1 ; c1ccc(-c2cc(-n3c4cc(-c5ccc6c7ccccc7n(-c7ccccc7)c6c5)ccc4c4ncccc43)nc(-c3ccccc3)n2)cc1 ; c1ccc(-c2cc(-c3ccccc3)nc(-n3c4cc(-c5ccc6c7ccccc7n(-c7ccccc7)c6c5)ccc4c4ncccc43)n2)cc1]

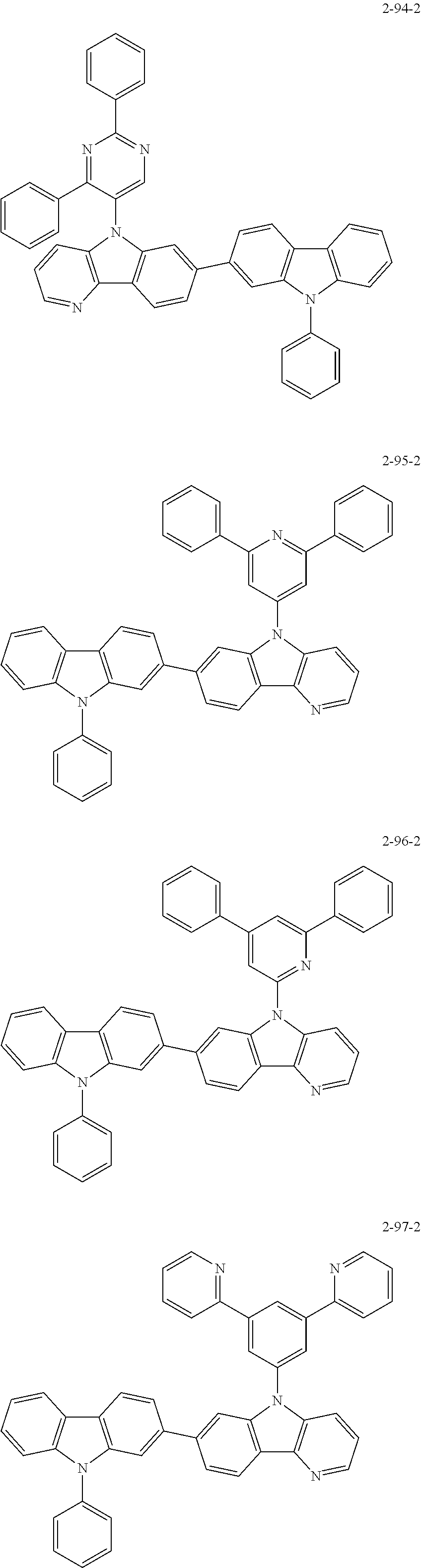 [SMILES: c1ccc(-c2ncc(-n3c4cc(-c5ccc6c7ccccc7n(-c7ccccc7)c6c5)ccc4c4ncccc43)c(-c3ccccc3)n2)cc1 ; c1ccc(-c2cc(-n3c4cc(-c5ccc6c7ccccc7n(-c7ccccc7)c6c5)ccc4c4ncccc43)cc(-c3ccccc3)n2)cc1 ; c1ccc(-c2cc(-c3ccccc3)nc(-n3c4cc(-c5ccc6c7ccccc7n(-c7ccccc7)c6c5)ccc4c4ncccc43)c2)cc1 ; c1ccc(-n2c3ccccc3c3ccc(-c4ccc5c6ncccc6n(-c6cc(-c7ccccn7)cc(-c7ccccn7)c6)c5c4)cc32)cc1]

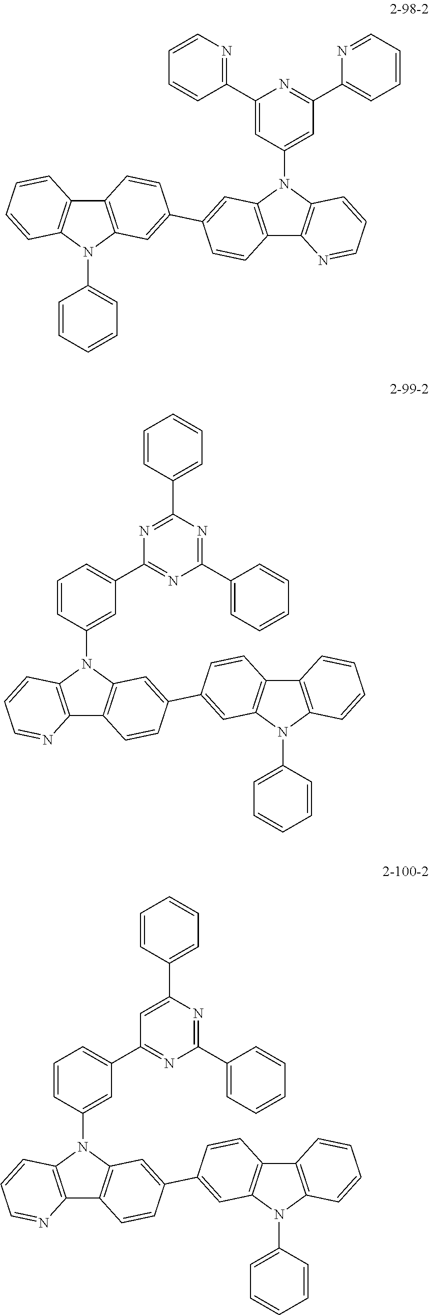 [SMILES: c1ccc(-n2c3ccccc3c3ccc(-c4ccc5c6ncccc6n(-c6cc(-c7ccccn7)nc(-c7ccccn7)c6)c5c4)cc32)cc1 ; c1ccc(-c2nc(-c3ccccc3)nc(-c3cccc(-n4c5cc(-c6ccc7c8ccccc8n(-c8ccccc8)c7c6)ccc5c5ncccc54)c3)n2)cc1 ; c1ccc(-c2cc(-c3cccc(-n4c5cc(-c6ccc7c8ccccc8n(-c8ccccc8)c7c6)ccc5c5ncccc54)c3)nc(-c3ccccc3)n2)cc1]

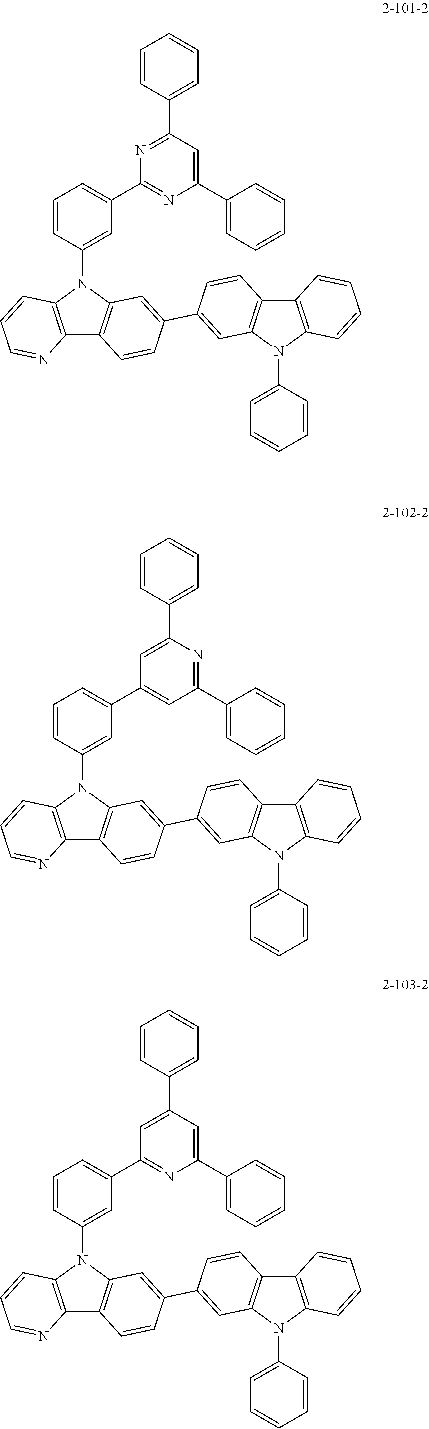 [SMILES: c1ccc(-c2cc(-c3ccccc3)nc(-c3cccc(-n4c5cc(-c6ccc7c8ccccc8n(-c8ccccc8)c7c6)ccc5c5ncccc54)c3)n2)cc1 ; c1ccc(-c2cc(-c3cccc(-n4c5cc(-c6ccc7c8ccccc8n(-c8ccccc8)c7c6)ccc5c5ncccc54)c3)cc(-c3ccccc3)n2)cc1 ; c1ccc(-c2cc(-c3ccccc3)nc(-c3cccc(-n4c5cc(-c6ccc7c8ccccc8n(-c8ccccc8)c7c6)ccc5c5ncccc54)c3)c2)cc1]

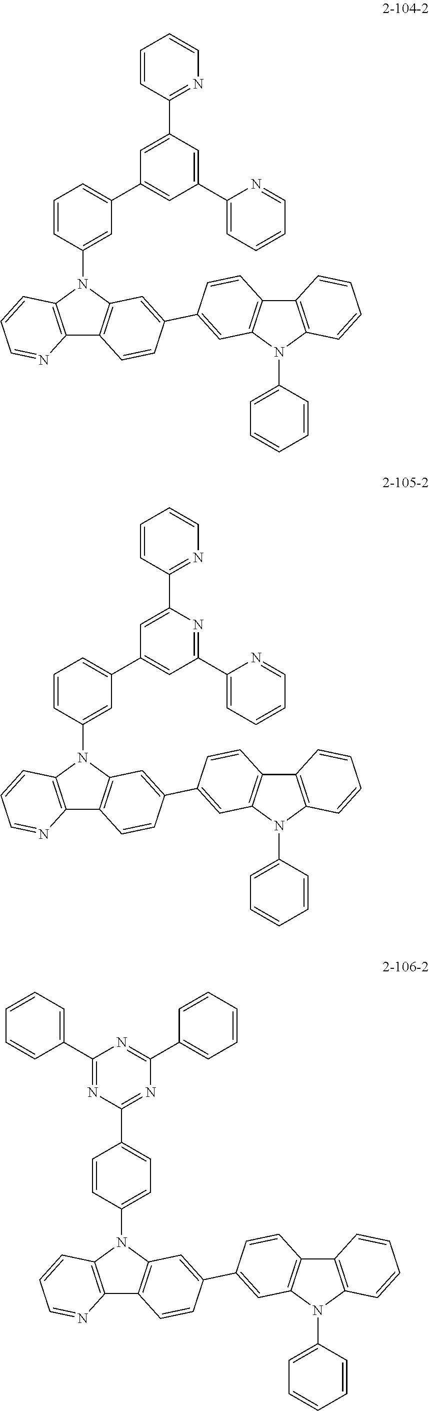 [SMILES: c1ccc(-n2c3ccccc3c3ccc(-c4ccc5c6ncccc6n(-c6cccc(-c7cc(-c8ccccn8)cc(-c8ccccn8)c7)c6)c5c4)cc32)cc1 ; c1ccc(-n2c3ccccc3c3ccc(-c4ccc5c6ncccc6n(-c6cccc(-c7cc(-c8ccccn8)nc(-c8ccccn8)c7)c6)c5c4)cc32)cc1 ; c1ccc(-c2nc(-c3ccccc3)nc(-c3ccc(-n4c5cc(-c6ccc7c8ccccc8n(-c8ccccc8)c7c6)ccc5c5ncccc54)cc3)n2)cc1]

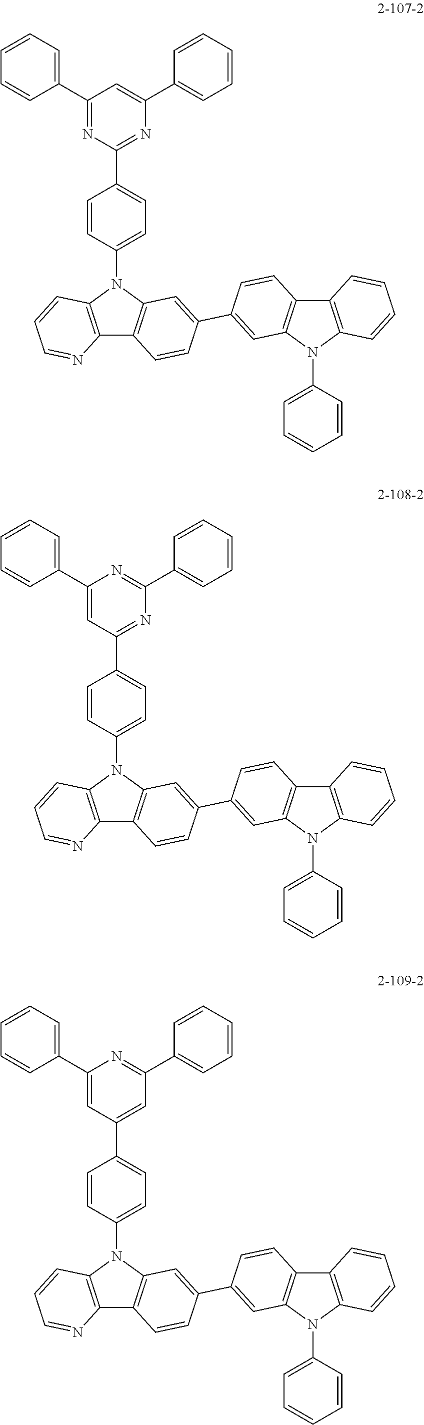 [SMILES: c1ccc(-c2cc(-c3ccccc3)nc(-c3ccc(-n4c5cc(-c6ccc7c8ccccc8n(-c8ccccc8)c7c6)ccc5c5ncccc54)cc3)n2)cc1 ; c1ccc(-c2cc(-c3ccc(-n4c5cc(-c6ccc7c8ccccc8n(-c8ccccc8)c7c6)ccc5c5ncccc54)cc3)nc(-c3ccccc3)n2)cc1 ; c1ccc(-c2cc(-c3ccc(-n4c5cc(-c6ccc7c8ccccc8n(-c8ccccc8)c7c6)ccc5c5ncccc54)cc3)cc(-c3ccccc3)n2)cc1]

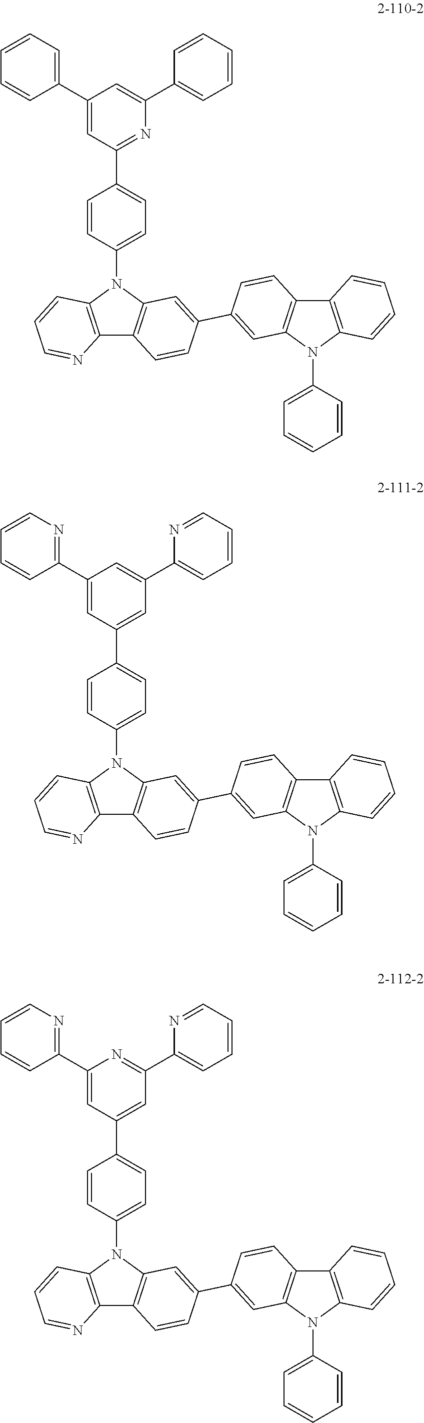 [SMILES: c1ccc(-c2cc(-c3ccccc3)nc(-c3ccc(-n4c5cc(-c6ccc7c8ccccc8n(-c8ccccc8)c7c6)ccc5c5ncccc54)cc3)c2)cc1 ; c1ccc(-n2c3ccccc3c3ccc(-c4ccc5c6ncccc6n(-c6ccc(-c7cc(-c8ccccn8)cc(-c8ccccn8)c7)cc6)c5c4)cc32)cc1 ; c1ccc(-n2c3ccccc3c3ccc(-c4ccc5c6ncccc6n(-c6ccc(-c7cc(-c8ccccn8)nc(-c8ccccn8)c7)cc6)c5c4)cc32)cc1]

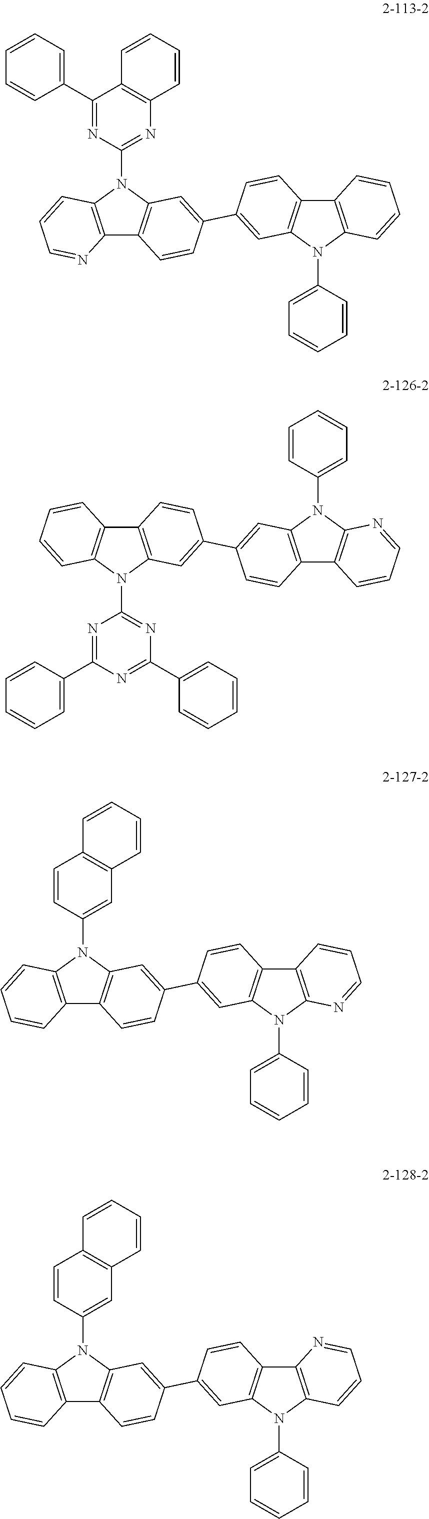 [SMILES: c1ccc(-c2nc(-n3c4cc(-c5ccc6c7ccccc7n(-c7ccccc7)c6c5)ccc4c4ncccc43)nc3ccccc23)cc1 ; c1ccc(-c2nc(-c3ccccc3)nc(-n3c4ccccc4c4ccc(-c5ccc6c7cccnc7n(-c7ccccc7)c6c5)cc43)n2)cc1 ; c1ccc(-n2c3cc(-c4ccc5c6ccccc6n(-c6ccc7ccccc7c6)c5c4)ccc3c3cccnc32)cc1 ; c1ccc(-n2c3cc(-c4ccc5c6ccccc6n(-c6ccc7ccccc7c6)c5c4)ccc3c3ncccc32)cc1]

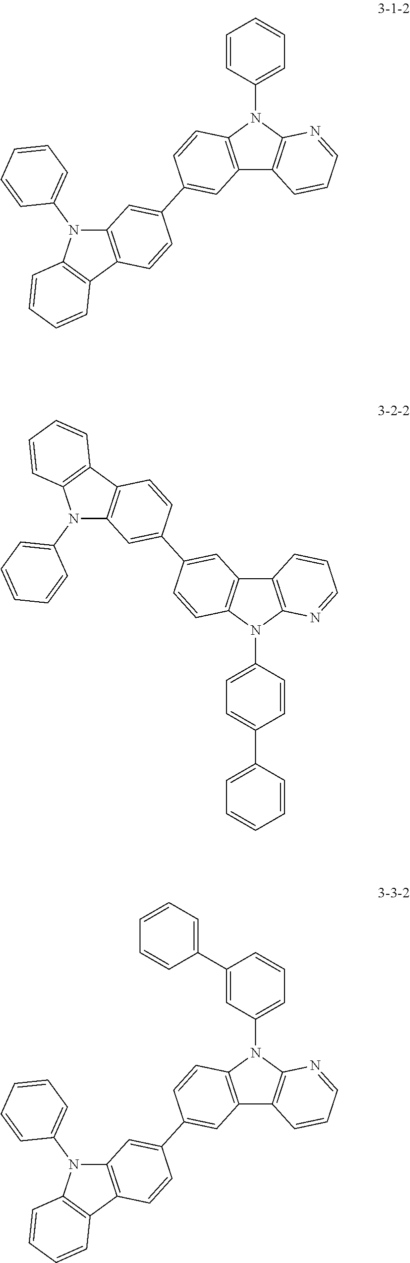 [SMILES: c1ccc(-n2c3ccccc3c3ccc(-c4ccc5c(c4)c4cccnc4n5-c4ccccc4)cc32)cc1 ; c1ccc(-c2ccc(-n3c4ccc(-c5ccc6c7ccccc7n(-c7ccccc7)c6c5)cc4c4cccnc43)cc2)cc1 ; c1ccc(-c2cccc(-n3c4ccc(-c5ccc6c7ccccc7n(-c7ccccc7)c6c5)cc4c4cccnc43)c2)cc1]

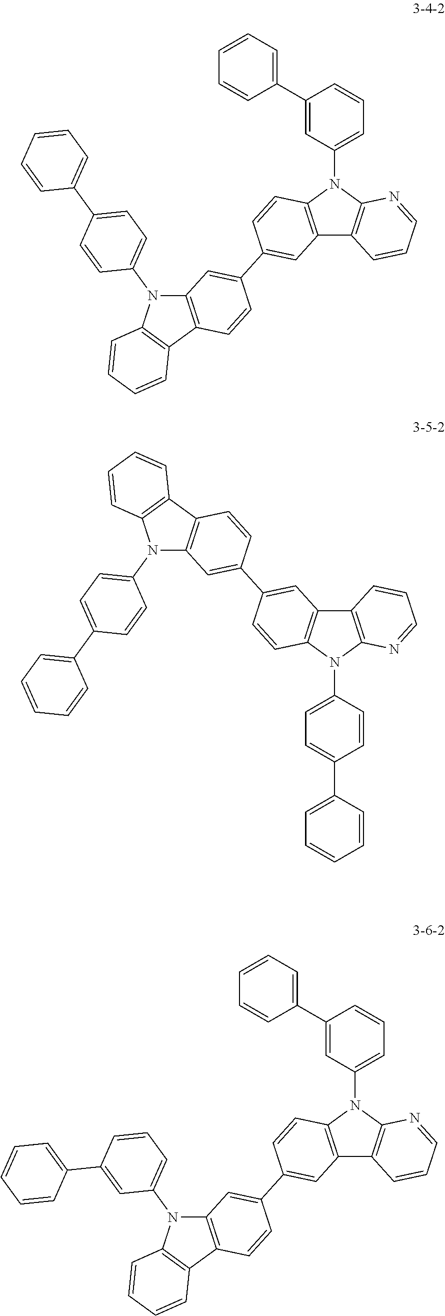 [SMILES: c1ccc(-c2ccc(-n3c4ccccc4c4ccc(-c5ccc6c(c5)c5cccnc5n6-c5cccc(-c6ccccc6)c5)cc43)cc2)cc1 ; c1ccc(-c2ccc(-n3c4ccccc4c4ccc(-c5ccc6c(c5)c5cccnc5n6-c5ccc(-c6ccccc6)cc5)cc43)cc2)cc1 ; c1ccc(-c2cccc(-n3c4ccccc4c4ccc(-c5ccc6c(c5)c5cccnc5n6-c5cccc(-c6ccccc6)c5)cc43)c2)cc1]

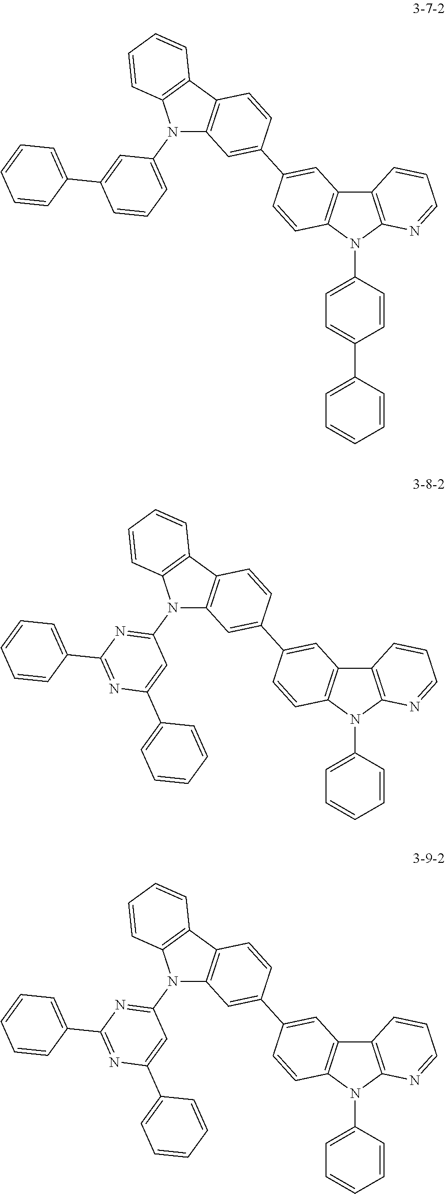 [SMILES: c1ccc(-c2ccc(-n3c4ccc(-c5ccc6c7ccccc7n(-c7cccc(-c8ccccc8)c7)c6c5)cc4c4cccnc43)cc2)cc1 ; c1ccc(-c2cc(-n3c4ccccc4c4ccc(-c5ccc6c(c5)c5cccnc5n6-c5ccccc5)cc43)nc(-c3ccccc3)n2)cc1 ; c1ccc(-c2cc(-n3c4ccccc4c4ccc(-c5ccc6c(c5)c5cccnc5n6-c5ccccc5)cc43)nc(-c3ccccc3)n2)cc1]

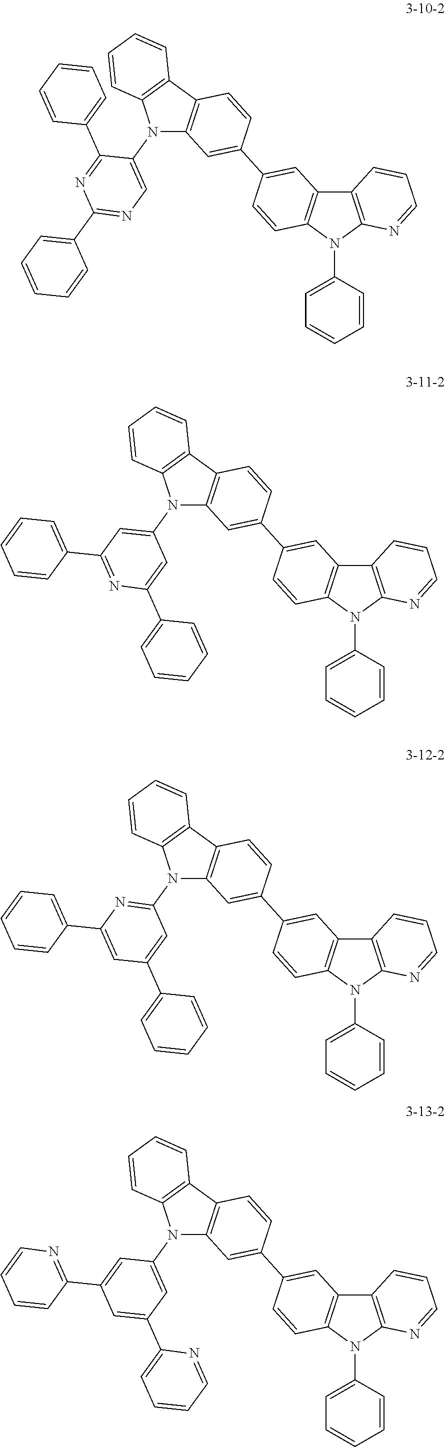 [SMILES: c1ccc(-c2ncc(-n3c4ccccc4c4ccc(-c5ccc6c(c5)c5cccnc5n6-c5ccccc5)cc43)c(-c3ccccc3)n2)cc1 ; c1ccc(-c2cc(-n3c4ccccc4c4ccc(-c5ccc6c(c5)c5cccnc5n6-c5ccccc5)cc43)cc(-c3ccccc3)n2)cc1 ; c1ccc(-c2cc(-c3ccccc3)nc(-n3c4ccccc4c4ccc(-c5ccc6c(c5)c5cccnc5n6-c5ccccc5)cc43)c2)cc1 ; c1ccc(-n2c3ccc(-c4ccc5c6ccccc6n(-c6cc(-c7ccccn7)cc(-c7ccccn7)c6)c5c4)cc3c3cccnc32)cc1]

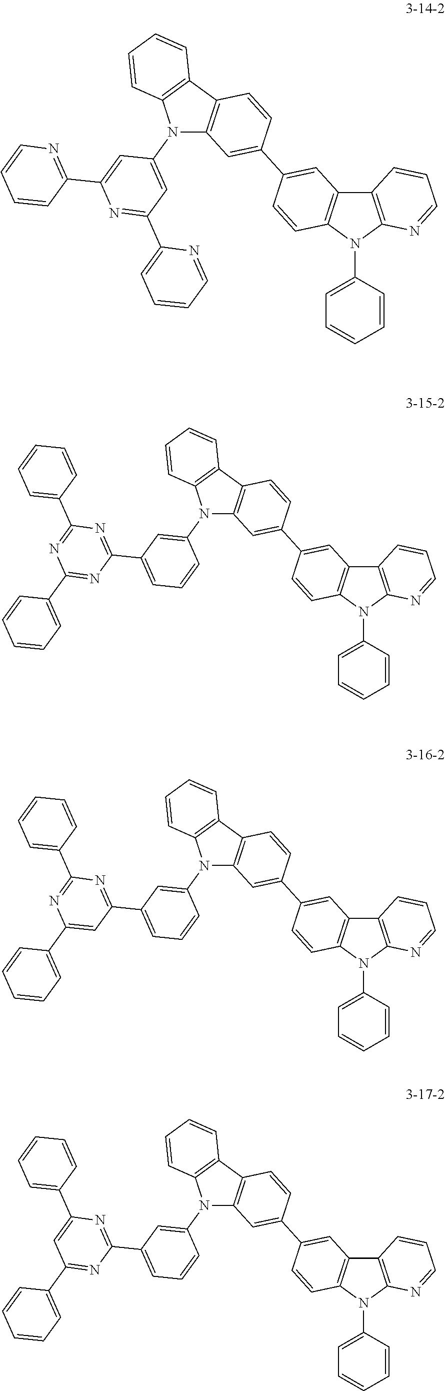 [SMILES: c1ccc(-n2c3ccc(-c4ccc5c6ccccc6n(-c6cc(-c7ccccn7)nc(-c7ccccn7)c6)c5c4)cc3c3cccnc32)cc1 ; c1ccc(-c2nc(-c3ccccc3)nc(-c3cccc(-n4c5ccccc5c5ccc(-c6ccc7c(c6)c6cccnc6n7-c6ccccc6)cc54)c3)n2)cc1 ; c1ccc(-c2cc(-c3cccc(-n4c5ccccc5c5ccc(-c6ccc7c(c6)c6cccnc6n7-c6ccccc6)cc54)c3)nc(-c3ccccc3)n2)cc1 ; c1ccc(-c2cc(-c3ccccc3)nc(-c3cccc(-n4c5ccccc5c5ccc(-c6ccc7c(c6)c6cccnc6n7-c6ccccc6)cc54)c3)n2)cc1]

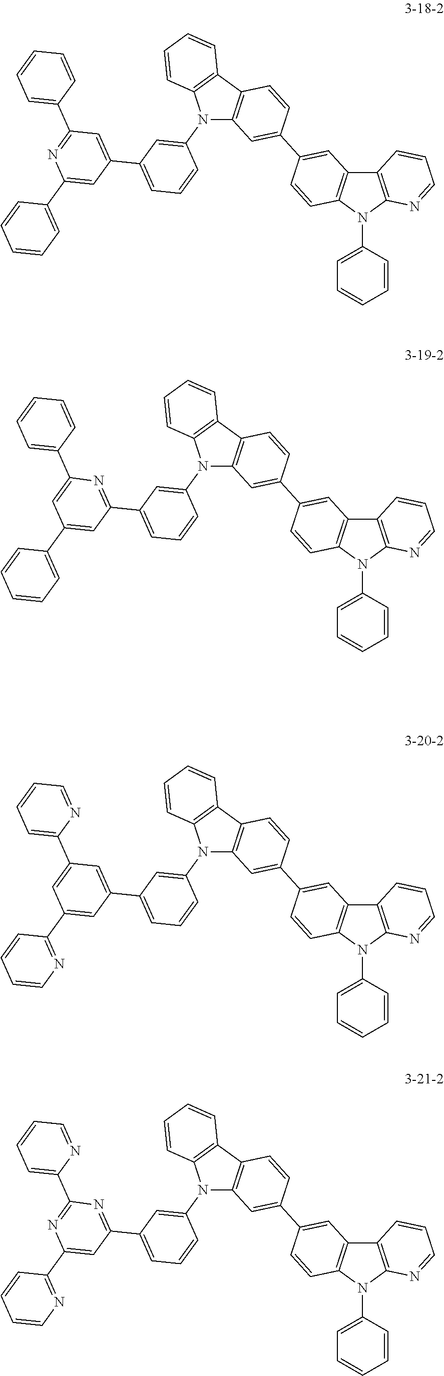 [SMILES: c1ccc(-c2cc(-c3cccc(-n4c5ccccc5c5ccc(-c6ccc7c(c6)c6cccnc6n7-c6ccccc6)cc54)c3)cc(-c3ccccc3)n2)cc1 ; c1ccc(-c2cc(-c3ccccc3)nc(-c3cccc(-n4c5ccccc5c5ccc(-c6ccc7c(c6)c6cccnc6n7-c6ccccc6)cc54)c3)c2)cc1 ; c1ccc(-n2c3ccc(-c4ccc5c6ccccc6n(-c6cccc(-c7cc(-c8ccccn8)cc(-c8ccccn8)c7)c6)c5c4)cc3c3cccnc32)cc1 ; c1ccc(-n2c3ccc(-c4ccc5c6ccccc6n(-c6cccc(-c7cc(-c8ccccn8)nc(-c8ccccn8)n7)c6)c5c4)cc3c3cccnc32)cc1]

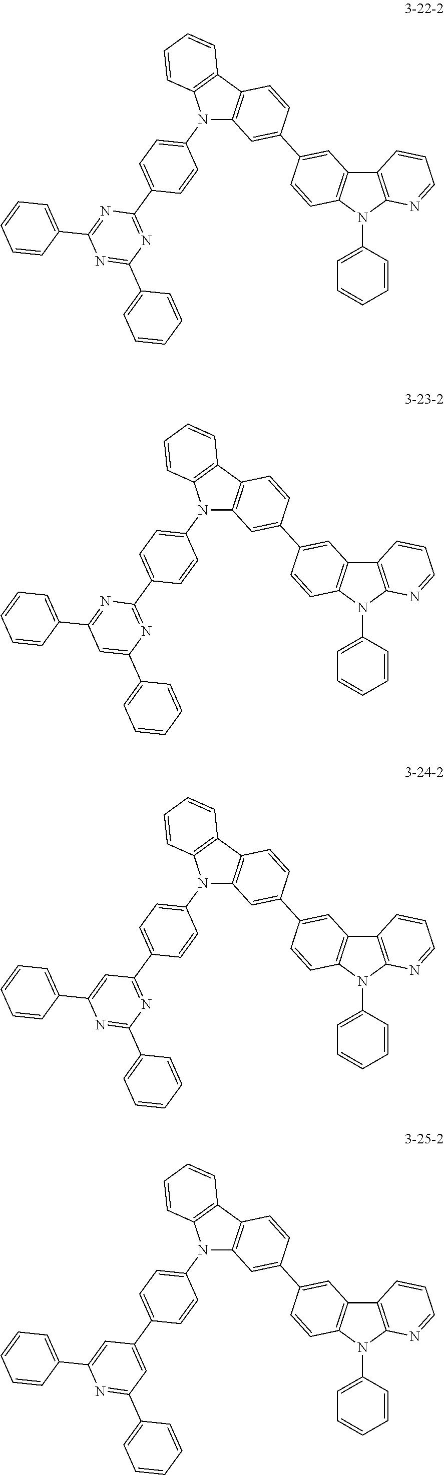 [SMILES: c1ccc(-c2nc(-c3ccccc3)nc(-c3ccc(-n4c5ccccc5c5ccc(-c6ccc7c(c6)c6cccnc6n7-c6ccccc6)cc54)cc3)n2)cc1 ; c1ccc(-c2cc(-c3ccccc3)nc(-c3ccc(-n4c5ccccc5c5ccc(-c6ccc7c(c6)c6cccnc6n7-c6ccccc6)cc54)cc3)n2)cc1 ; c1ccc(-c2cc(-c3ccc(-n4c5ccccc5c5ccc(-c6ccc7c(c6)c6cccnc6n7-c6ccccc6)cc54)cc3)nc(-c3ccccc3)n2)cc1 ; c1ccc(-c2cc(-c3ccc(-n4c5ccccc5c5ccc(-c6ccc7c(c6)c6cccnc6n7-c6ccccc6)cc54)cc3)cc(-c3ccccc3)n2)cc1]

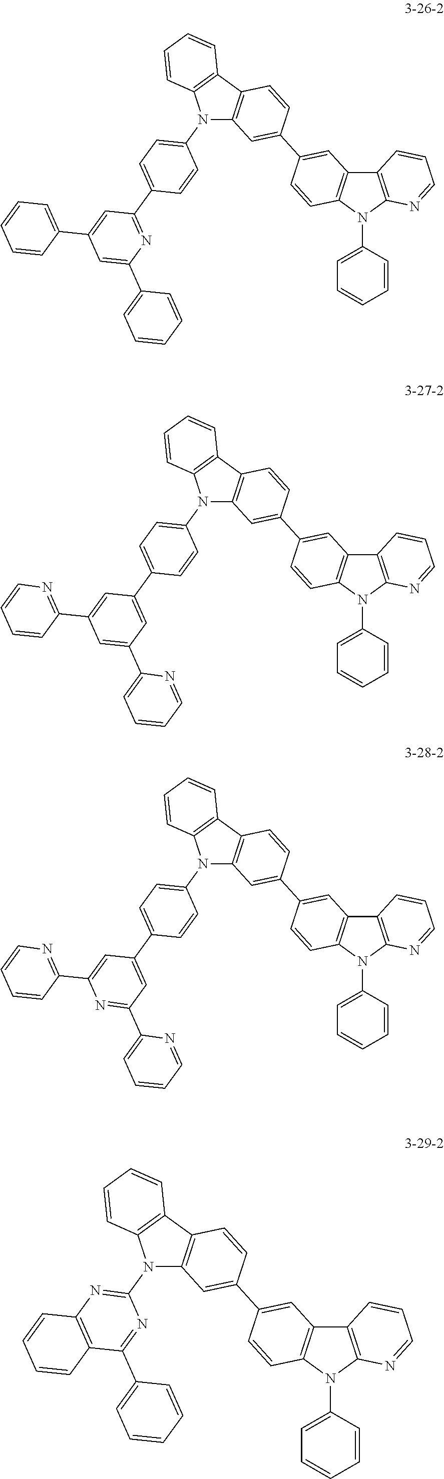 [SMILES: c1ccc(-c2cc(-c3ccccc3)nc(-c3ccc(-n4c5ccccc5c5ccc(-c6ccc7c(c6)c6cccnc6n7-c6ccccc6)cc54)cc3)c2)cc1 ; c1ccc(-n2c3ccc(-c4ccc5c6ccccc6n(-c6ccc(-c7cc(-c8ccccn8)cc(-c8ccccn8)c7)cc6)c5c4)cc3c3cccnc32)cc1 ; c1ccc(-n2c3ccc(-c4ccc5c6ccccc6n(-c6ccc(-c7cc(-c8ccccn8)nc(-c8ccccn8)c7)cc6)c5c4)cc3c3cccnc32)cc1 ; c1ccc(-c2nc(-n3c4ccccc4c4ccc(-c5ccc6c(c5)c5cccnc5n6-c5ccccc5)cc43)nc3ccccc23)cc1]

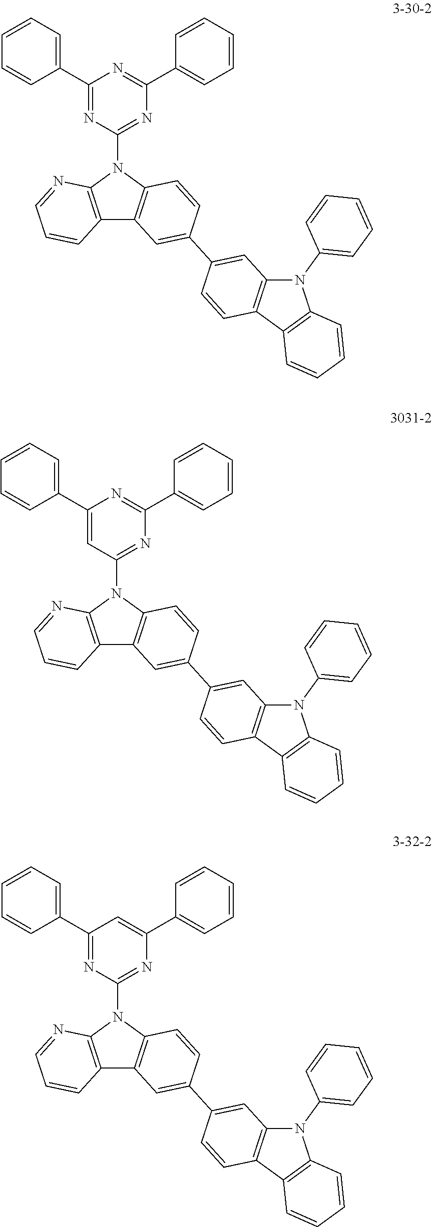 [SMILES: c1ccc(-c2nc(-c3ccccc3)nc(-n3c4ccc(-c5ccc6c7ccccc7n(-c7ccccc7)c6c5)cc4c4cccnc43)n2)cc1 ; c1ccc(-c2cc(-n3c4ccc(-c5ccc6c7ccccc7n(-c7ccccc7)c6c5)cc4c4cccnc43)nc(-c3ccccc3)n2)cc1 ; c1ccc(-c2cc(-c3ccccc3)nc(-n3c4ccc(-c5ccc6c7ccccc7n(-c7ccccc7)c6c5)cc4c4cccnc43)n2)cc1]

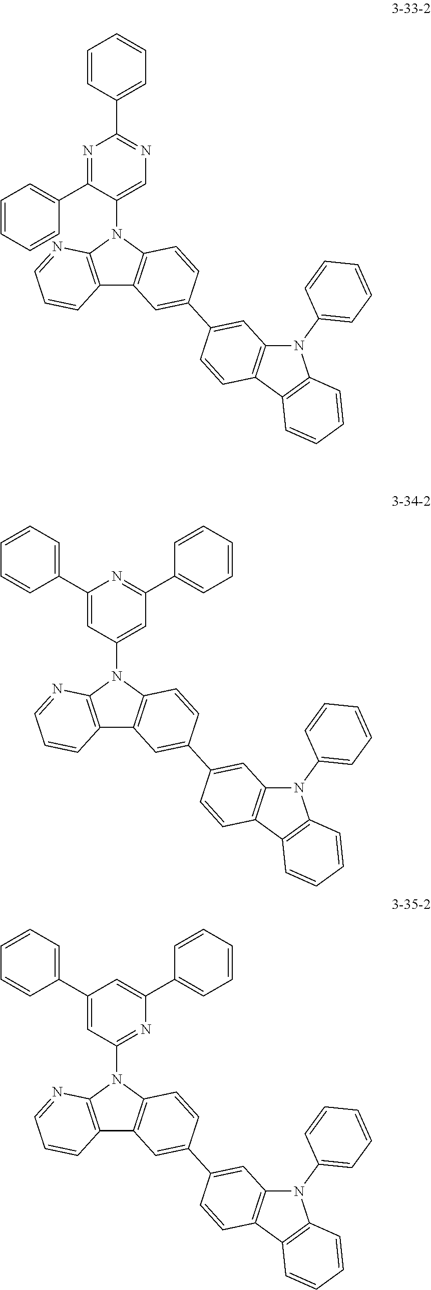 [SMILES: c1ccc(-c2ncc(-n3c4ccc(-c5ccc6c7ccccc7n(-c7ccccc7)c6c5)cc4c4cccnc43)c(-c3ccccc3)n2)cc1 ; c1ccc(-c2cc(-n3c4ccc(-c5ccc6c7ccccc7n(-c7ccccc7)c6c5)cc4c4cccnc43)cc(-c3ccccc3)n2)cc1 ; c1ccc(-c2cc(-c3ccccc3)nc(-n3c4ccc(-c5ccc6c7ccccc7n(-c7ccccc7)c6c5)cc4c4cccnc43)c2)cc1]

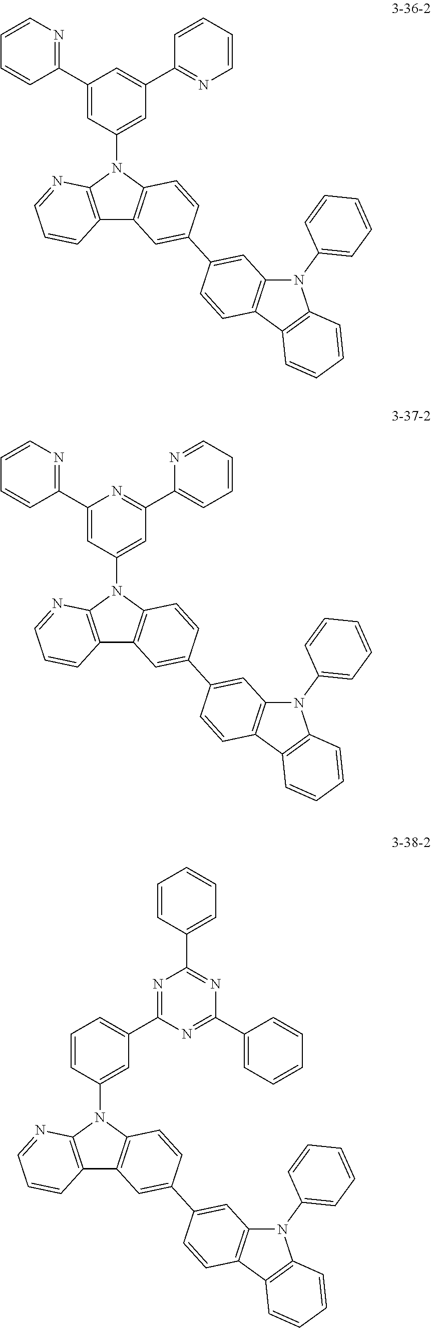 [SMILES: c1ccc(-n2c3ccccc3c3ccc(-c4ccc5c(c4)c4cccnc4n5-c4cc(-c5ccccn5)cc(-c5ccccn5)c4)cc32)cc1 ; c1ccc(-n2c3ccccc3c3ccc(-c4ccc5c(c4)c4cccnc4n5-c4cc(-c5ccccn5)nc(-c5ccccn5)c4)cc32)cc1 ; c1ccc(-c2nc(-c3ccccc3)nc(-c3cccc(-n4c5ccc(-c6ccc7c8ccccc8n(-c8ccccc8)c7c6)cc5c5cccnc54)c3)n2)cc1]

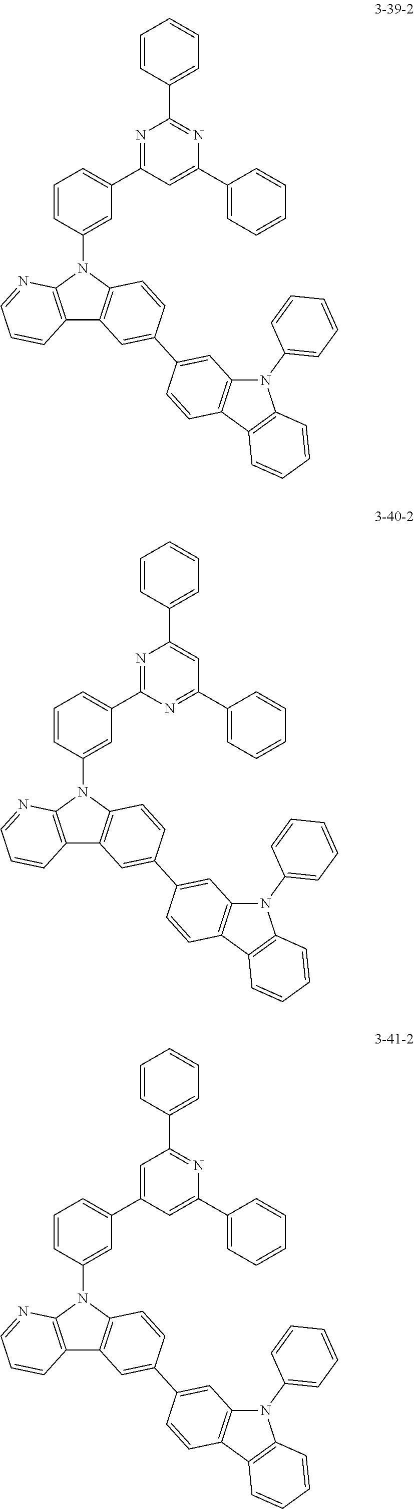 [SMILES: c1ccc(-c2cc(-c3cccc(-n4c5ccc(-c6ccc7c8ccccc8n(-c8ccccc8)c7c6)cc5c5cccnc54)c3)nc(-c3ccccc3)n2)cc1 ; c1ccc(-c2cc(-c3ccccc3)nc(-c3cccc(-n4c5ccc(-c6ccc7c8ccccc8n(-c8ccccc8)c7c6)cc5c5cccnc54)c3)n2)cc1 ; c1ccc(-c2cc(-c3cccc(-n4c5ccc(-c6ccc7c8ccccc8n(-c8ccccc8)c7c6)cc5c5cccnc54)c3)cc(-c3ccccc3)n2)cc1]

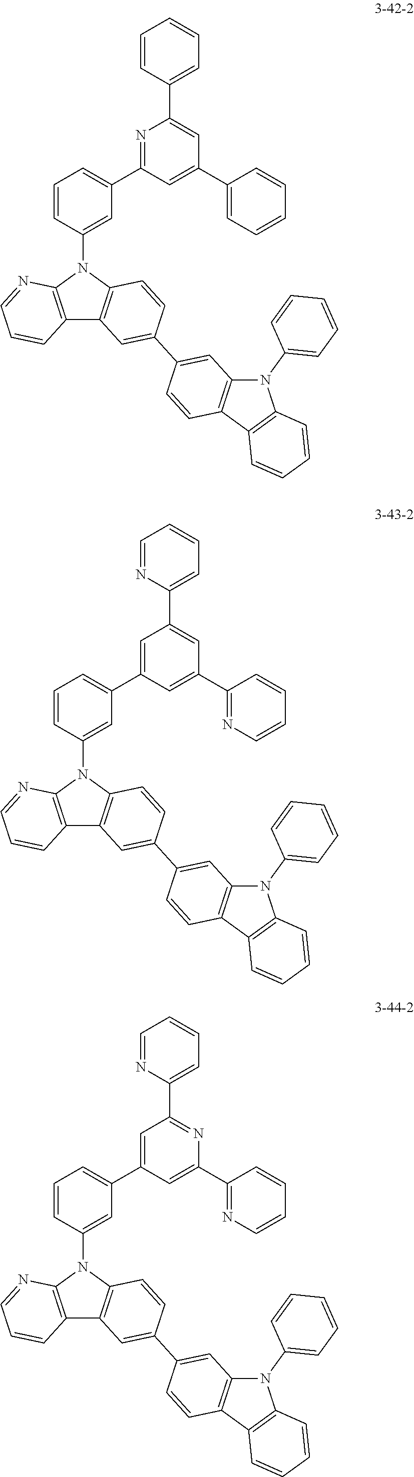 [SMILES: c1ccc(-c2cc(-c3ccccc3)nc(-c3cccc(-n4c5ccc(-c6ccc7c8ccccc8n(-c8ccccc8)c7c6)cc5c5cccnc54)c3)c2)cc1 ; c1ccc(-n2c3ccccc3c3ccc(-c4ccc5c(c4)c4cccnc4n5-c4cccc(-c5cc(-c6ccccn6)cc(-c6ccccn6)c5)c4)cc32)cc1 ; c1ccc(-n2c3ccccc3c3ccc(-c4ccc5c(c4)c4cccnc4n5-c4cccc(-c5cc(-c6ccccn6)nc(-c6ccccn6)c5)c4)cc32)cc1]

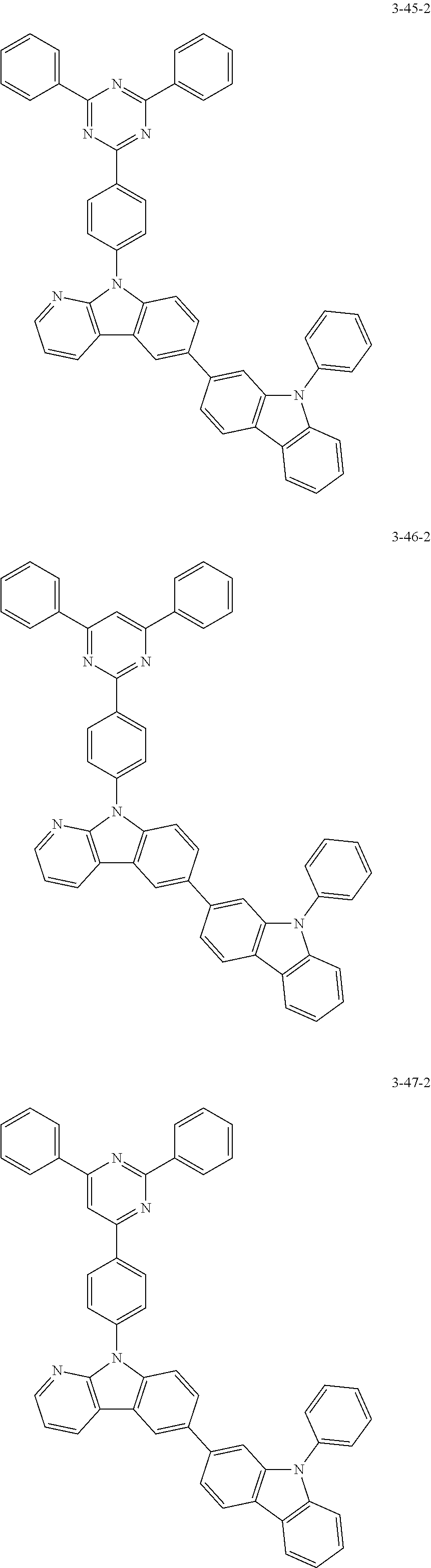 [SMILES: c1ccc(-c2nc(-c3ccccc3)nc(-c3ccc(-n4c5ccc(-c6ccc7c8ccccc8n(-c8ccccc8)c7c6)cc5c5cccnc54)cc3)n2)cc1 ; c1ccc(-c2cc(-c3ccccc3)nc(-c3ccc(-n4c5ccc(-c6ccc7c8ccccc8n(-c8ccccc8)c7c6)cc5c5cccnc54)cc3)n2)cc1 ; c1ccc(-c2cc(-c3ccc(-n4c5ccc(-c6ccc7c8ccccc8n(-c8ccccc8)c7c6)cc5c5cccnc54)cc3)nc(-c3ccccc3)n2)cc1]

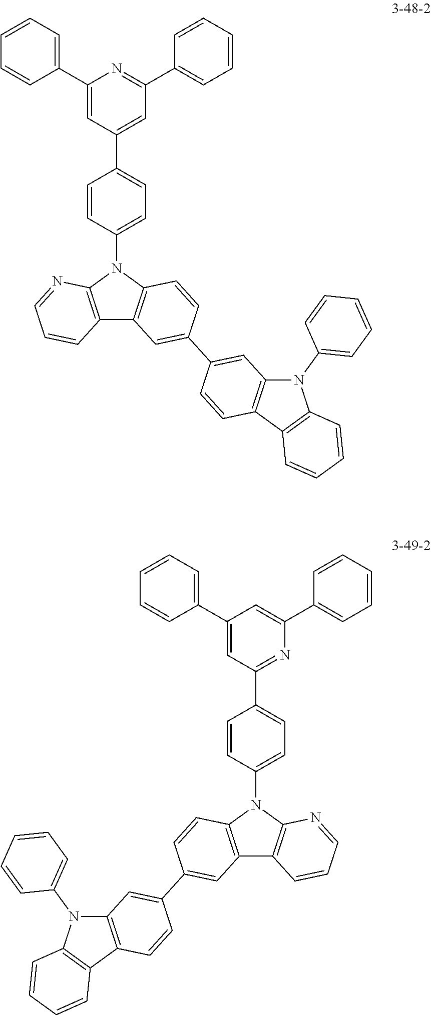 [SMILES: c1ccc(-c2cc(-c3ccc(-n4c5ccc(-c6ccc7c8ccccc8n(-c8ccccc8)c7c6)cc5c5cccnc54)cc3)cc(-c3ccccc3)n2)cc1 ; c1ccc(-c2cc(-c3ccccc3)nc(-c3ccc(-n4c5ccc(-c6ccc7c8ccccc8n(-c8ccccc8)c7c6)cc5c5cccnc54)cc3)c2)cc1]

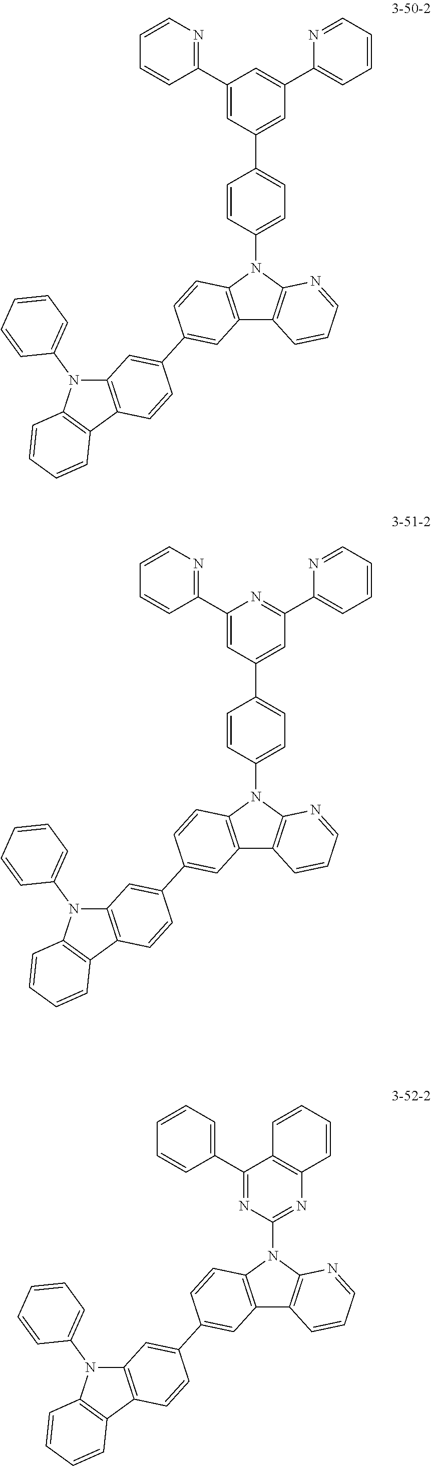 [SMILES: c1ccc(-n2c3ccccc3c3ccc(-c4ccc5c(c4)c4cccnc4n5-c4ccc(-c5cc(-c6ccccn6)cc(-c6ccccn6)c5)cc4)cc32)cc1 ; c1ccc(-n2c3ccccc3c3ccc(-c4ccc5c(c4)c4cccnc4n5-c4ccc(-c5cc(-c6ccccn6)nc(-c6ccccn6)c5)cc4)cc32)cc1 ; c1ccc(-c2nc(-n3c4ccc(-c5ccc6c7ccccc7n(-c7ccccc7)c6c5)cc4c4cccnc43)nc3ccccc23)cc1]

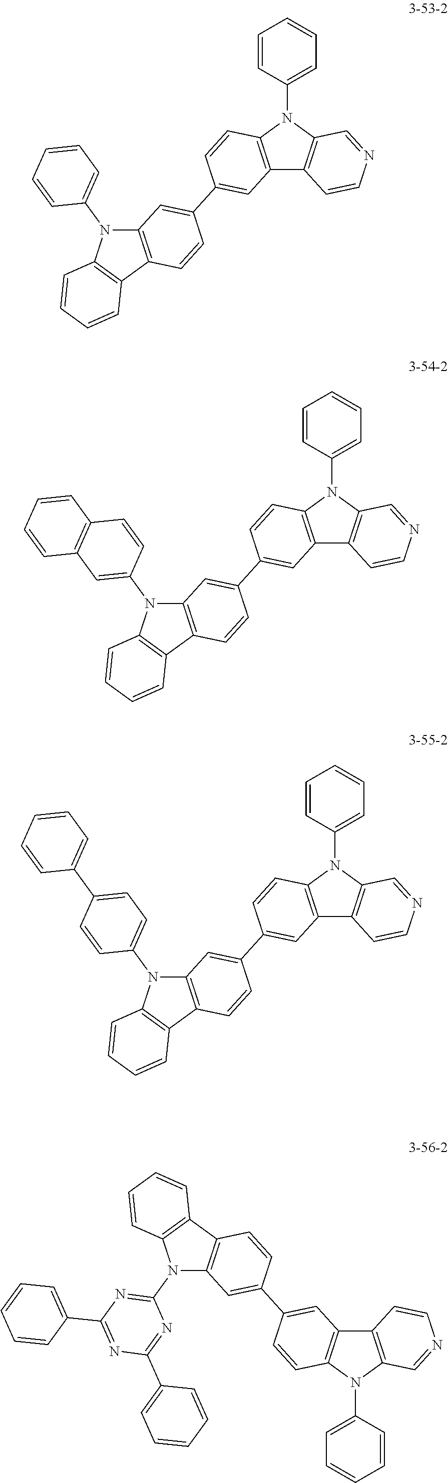 [SMILES: c1ccc(-n2c3ccc(-c4ccc5c6ccccc6n(-c6ccccc6)c5c4)cc3c3ccncc32)cc1 ; c1ccc(-n2c3ccc(-c4ccc5c6ccccc6n(-c6ccc7ccccc7c6)c5c4)cc3c3ccncc32)cc1 ; c1ccc(-c2ccc(-n3c4ccccc4c4ccc(-c5ccc6c(c5)c5ccncc5n6-c5ccccc5)cc43)cc2)cc1 ; c1ccc(-c2nc(-c3ccccc3)nc(-n3c4ccccc4c4ccc(-c5ccc6c(c5)c5ccncc5n6-c5ccccc5)cc43)n2)cc1]

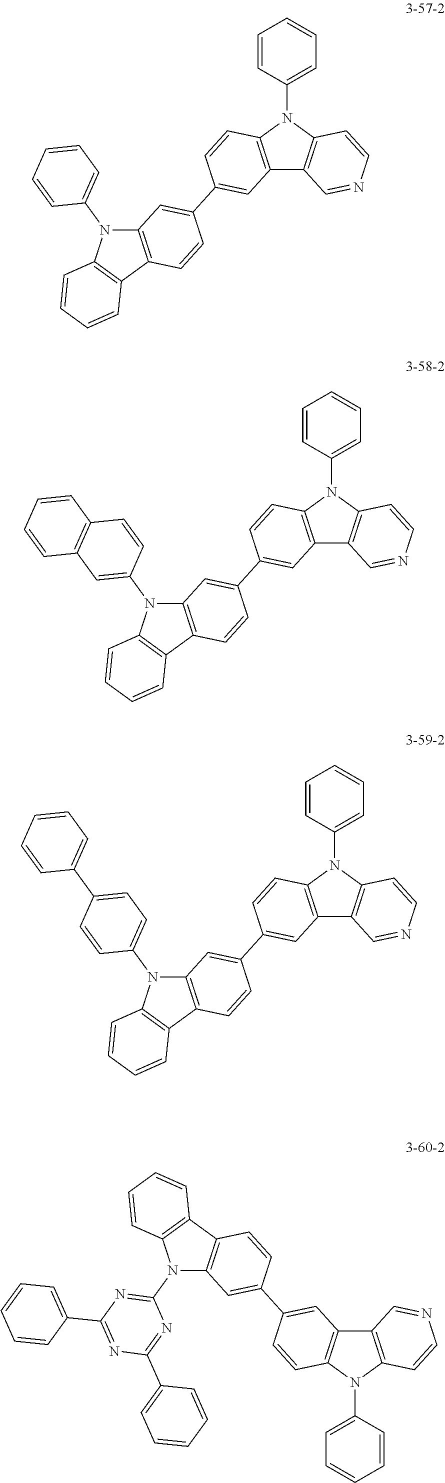 [SMILES: c1ccc(-n2c3ccncc3c3cc(-c4ccc5c6ccccc6n(-c6ccccc6)c5c4)ccc32)cc1 ; c1ccc(-n2c3ccncc3c3cc(-c4ccc5c6ccccc6n(-c6ccc7ccccc7c6)c5c4)ccc32)cc1 ; c1ccc(-c2ccc(-n3c4ccccc4c4ccc(-c5ccc6c(c5)c5cnccc5n6-c5ccccc5)cc43)cc2)cc1 ; c1ccc(-c2nc(-c3ccccc3)nc(-n3c4ccccc4c4ccc(-c5ccc6c(c5)c5cnccc5n6-c5ccccc5)cc43)n2)cc1]

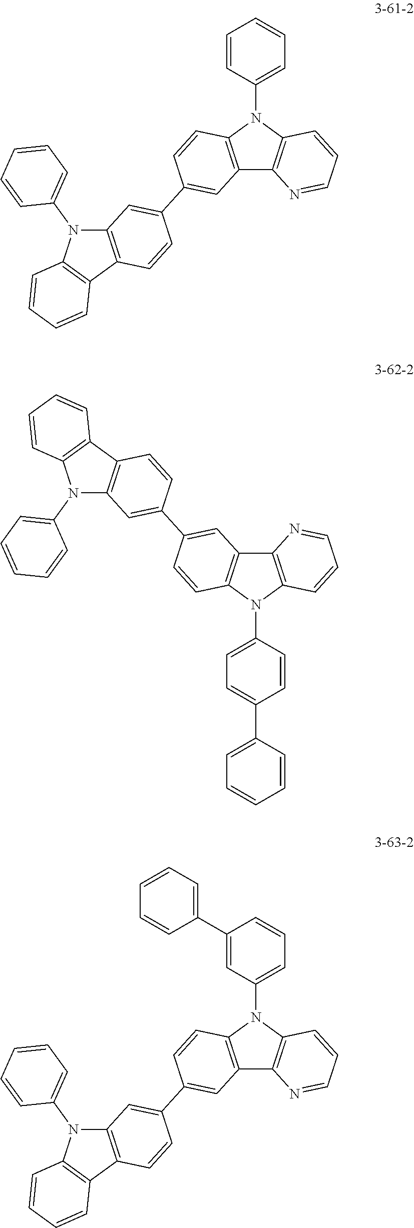 [SMILES: c1ccc(-n2c3ccccc3c3ccc(-c4ccc5c(c4)c4ncccc4n5-c4ccccc4)cc32)cc1 ; c1ccc(-c2ccc(-n3c4ccc(-c5ccc6c7ccccc7n(-c7ccccc7)c6c5)cc4c4ncccc43)cc2)cc1 ; c1ccc(-c2cccc(-n3c4ccc(-c5ccc6c7ccccc7n(-c7ccccc7)c6c5)cc4c4ncccc43)c2)cc1]

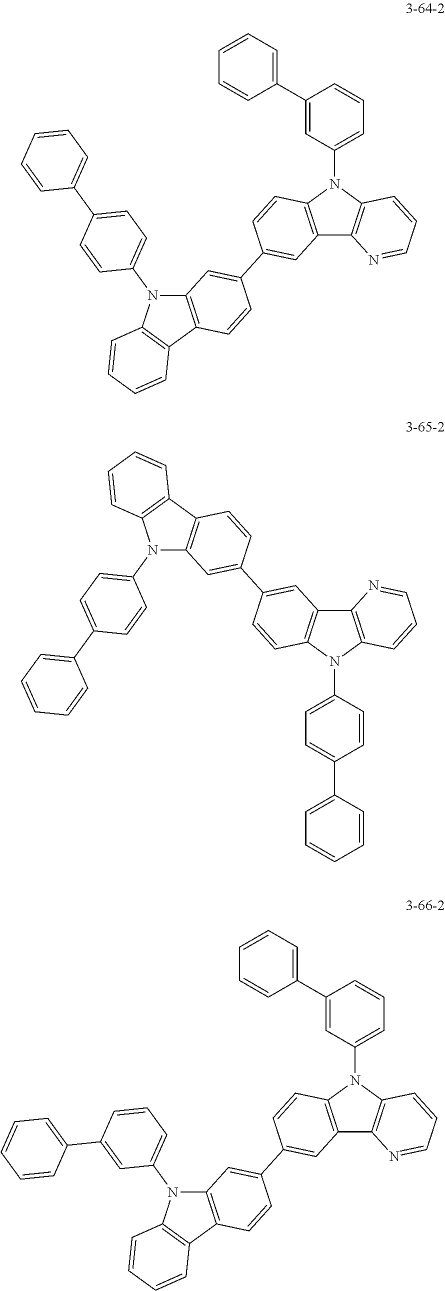 [SMILES: c1ccc(-c2ccc(-n3c4ccccc4c4ccc(-c5ccc6c(c5)c5ncccc5n6-c5cccc(-c6ccccc6)c5)cc43)cc2)cc1 ; c1ccc(-c2ccc(-n3c4ccccc4c4ccc(-c5ccc6c(c5)c5ncccc5n6-c5ccc(-c6ccccc6)cc5)cc43)cc2)cc1 ; c1ccc(-c2cccc(-n3c4ccccc4c4ccc(-c5ccc6c(c5)c5ncccc5n6-c5cccc(-c6ccccc6)c5)cc43)c2)cc1]

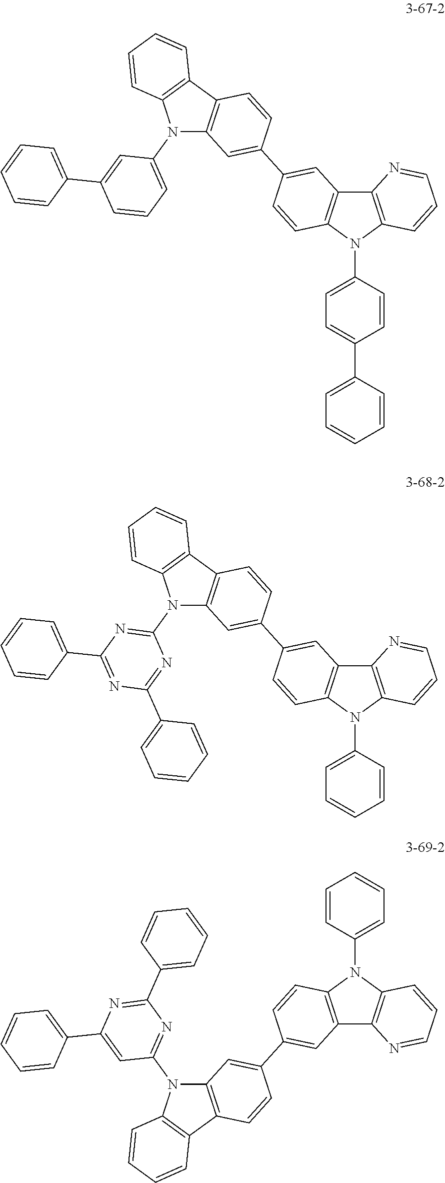 [SMILES: c1ccc(-c2ccc(-n3c4ccc(-c5ccc6c7ccccc7n(-c7cccc(-c8ccccc8)c7)c6c5)cc4c4ncccc43)cc2)cc1 ; c1ccc(-c2nc(-c3ccccc3)nc(-n3c4ccccc4c4ccc(-c5ccc6c(c5)c5ncccc5n6-c5ccccc5)cc43)n2)cc1 ; c1ccc(-c2cc(-n3c4ccccc4c4ccc(-c5ccc6c(c5)c5ncccc5n6-c5ccccc5)cc43)nc(-c3ccccc3)n2)cc1]

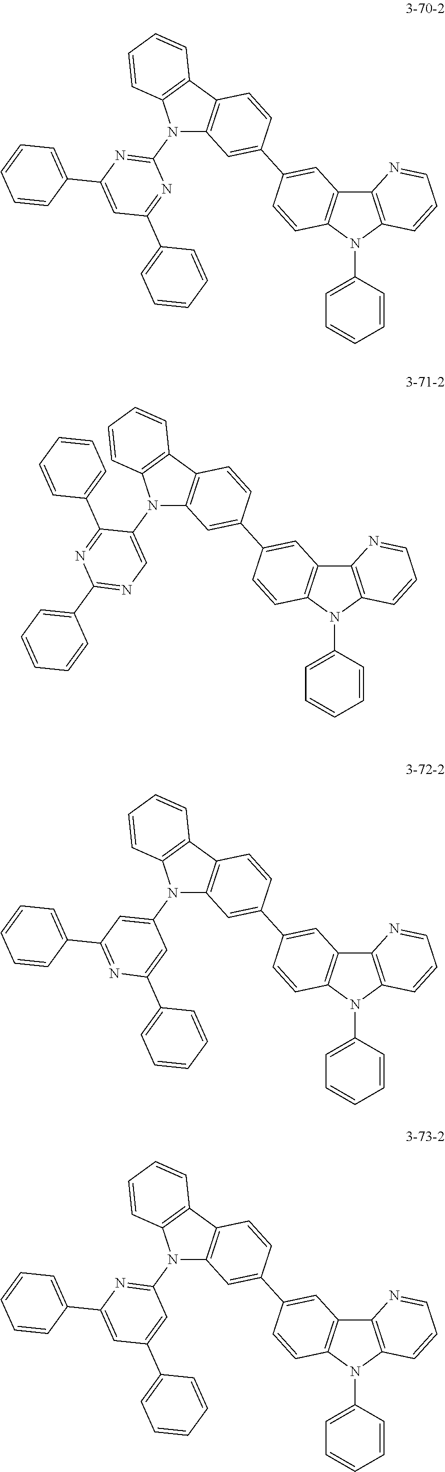 [SMILES: c1ccc(-c2cc(-c3ccccc3)nc(-n3c4ccccc4c4ccc(-c5ccc6c(c5)c5ncccc5n6-c5ccccc5)cc43)n2)cc1 ; c1ccc(-c2ncc(-n3c4ccccc4c4ccc(-c5ccc6c(c5)c5ncccc5n6-c5ccccc5)cc43)c(-c3ccccc3)n2)cc1 ; c1ccc(-c2cc(-n3c4ccccc4c4ccc(-c5ccc6c(c5)c5ncccc5n6-c5ccccc5)cc43)cc(-c3ccccc3)n2)cc1 ; c1ccc(-c2cc(-c3ccccc3)nc(-n3c4ccccc4c4ccc(-c5ccc6c(c5)c5ncccc5n6-c5ccccc5)cc43)c2)cc1]

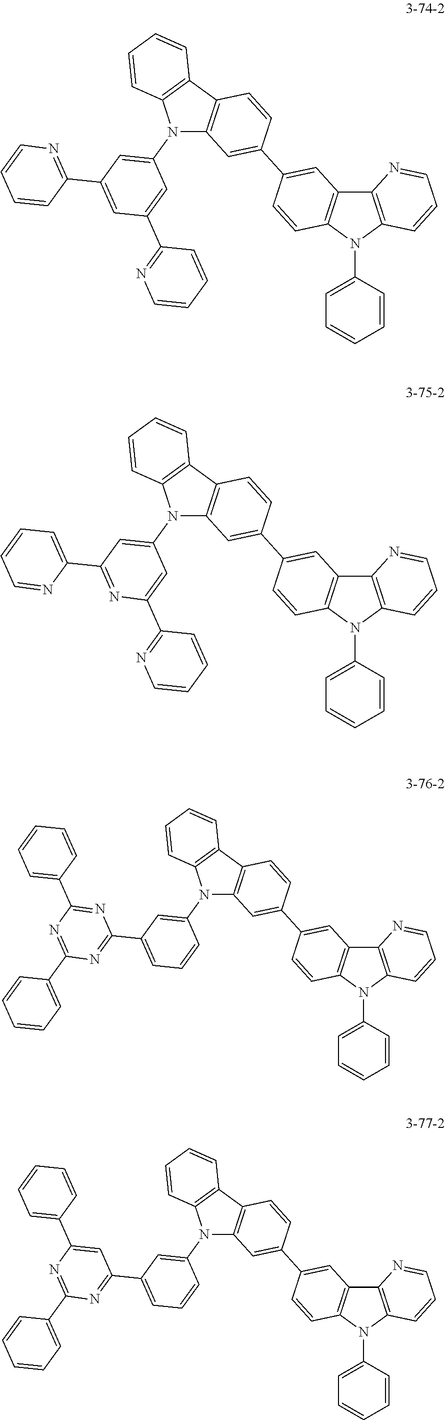 [SMILES: c1ccc(-n2c3ccc(-c4ccc5c6ccccc6n(-c6cc(-c7ccccn7)cc(-c7ccccn7)c6)c5c4)cc3c3ncccc32)cc1 ; c1ccc(-n2c3ccc(-c4ccc5c6ccccc6n(-c6cc(-c7ccccn7)nc(-c7ccccn7)c6)c5c4)cc3c3ncccc32)cc1 ; c1ccc(-c2nc(-c3ccccc3)nc(-c3cccc(-n4c5ccccc5c5ccc(-c6ccc7c(c6)c6ncccc6n7-c6ccccc6)cc54)c3)n2)cc1 ; c1ccc(-c2cc(-c3cccc(-n4c5ccccc5c5ccc(-c6ccc7c(c6)c6ncccc6n7-c6ccccc6)cc54)c3)nc(-c3ccccc3)n2)cc1]

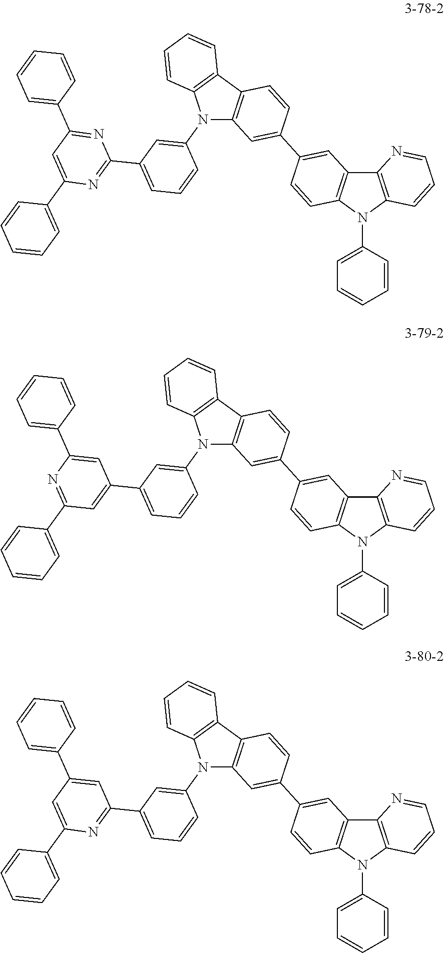 [SMILES: c1ccc(-c2cc(-c3ccccc3)nc(-c3cccc(-n4c5ccccc5c5ccc(-c6ccc7c(c6)c6ncccc6n7-c6ccccc6)cc54)c3)n2)cc1 ; c1ccc(-c2cc(-c3cccc(-n4c5ccccc5c5ccc(-c6ccc7c(c6)c6ncccc6n7-c6ccccc6)cc54)c3)cc(-c3ccccc3)n2)cc1 ; c1ccc(-c2cc(-c3ccccc3)nc(-c3cccc(-n4c5ccccc5c5ccc(-c6ccc7c(c6)c6ncccc6n7-c6ccccc6)cc54)c3)c2)cc1]

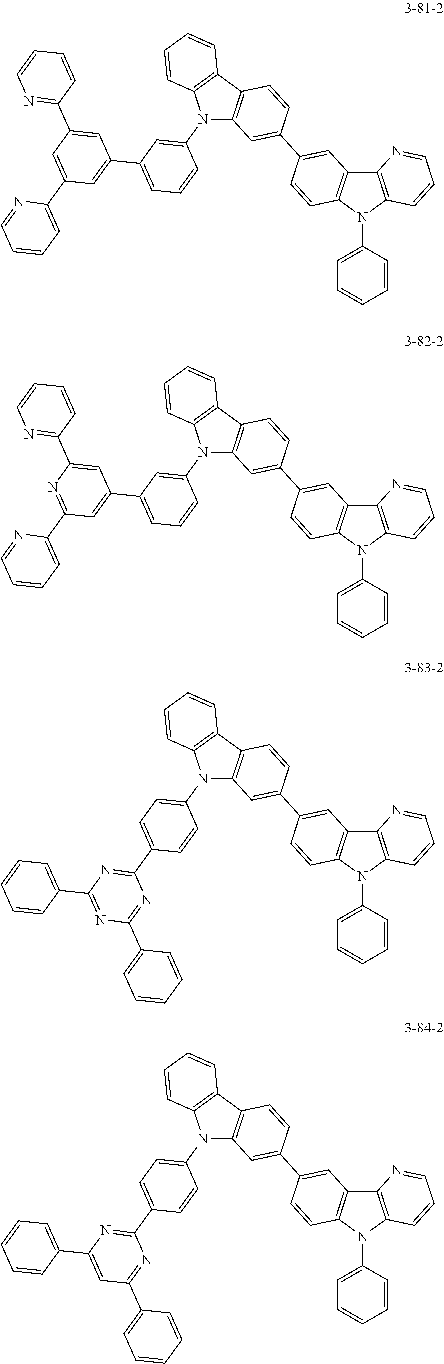 [SMILES: c1ccc(-n2c3ccc(-c4ccc5c6ccccc6n(-c6cccc(-c7cc(-c8ccccn8)cc(-c8ccccn8)c7)c6)c5c4)cc3c3ncccc32)cc1 ; c1ccc(-n2c3ccc(-c4ccc5c6ccccc6n(-c6cccc(-c7cc(-c8ccccn8)nc(-c8ccccn8)c7)c6)c5c4)cc3c3ncccc32)cc1 ; c1ccc(-c2nc(-c3ccccc3)nc(-c3ccc(-n4c5ccccc5c5ccc(-c6ccc7c(c6)c6ncccc6n7-c6ccccc6)cc54)cc3)n2)cc1 ; c1ccc(-c2cc(-c3ccccc3)nc(-c3ccc(-n4c5ccccc5c5ccc(-c6ccc7c(c6)c6ncccc6n7-c6ccccc6)cc54)cc3)n2)cc1]

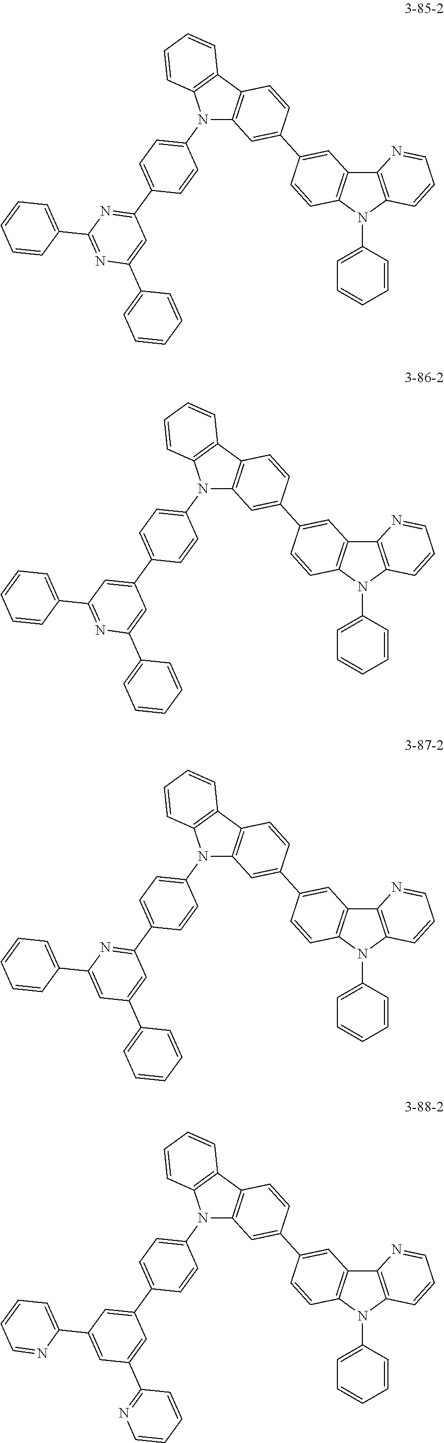 [SMILES: c1ccc(-c2cc(-c3ccc(-n4c5ccccc5c5ccc(-c6ccc7c(c6)c6ncccc6n7-c6ccccc6)cc54)cc3)nc(-c3ccccc3)n2)cc1 ; c1ccc(-c2cc(-c3ccc(-n4c5ccccc5c5ccc(-c6ccc7c(c6)c6ncccc6n7-c6ccccc6)cc54)cc3)cc(-c3ccccc3)n2)cc1 ; c1ccc(-c2cc(-c3ccccc3)nc(-c3ccc(-n4c5ccccc5c5ccc(-c6ccc7c(c6)c6ncccc6n7-c6ccccc6)cc54)cc3)c2)cc1 ; c1ccc(-n2c3ccc(-c4ccc5c6ccccc6n(-c6ccc(-c7cc(-c8ccccn8)cc(-c8ccccn8)c7)cc6)c5c4)cc3c3ncccc32)cc1]

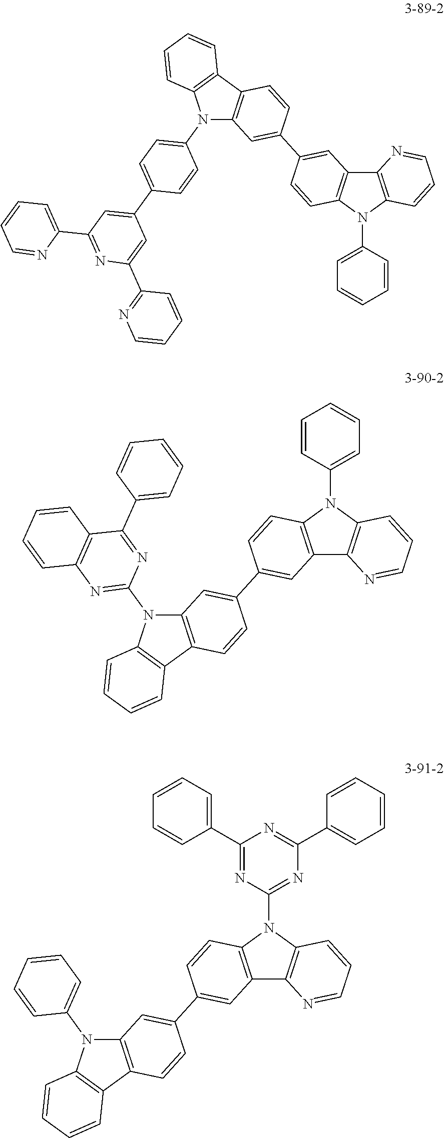 [SMILES: c1ccc(-n2c3ccc(-c4ccc5c6ccccc6n(-c6ccc(-c7cc(-c8ccccn8)nc(-c8ccccn8)c7)cc6)c5c4)cc3c3ncccc32)cc1 ; c1ccc(-c2nc(-n3c4ccccc4c4ccc(-c5ccc6c(c5)c5ncccc5n6-c5ccccc5)cc43)nc3ccccc23)cc1 ; c1ccc(-c2nc(-c3ccccc3)nc(-n3c4ccc(-c5ccc6c7ccccc7n(-c7ccccc7)c6c5)cc4c4ncccc43)n2)cc1]

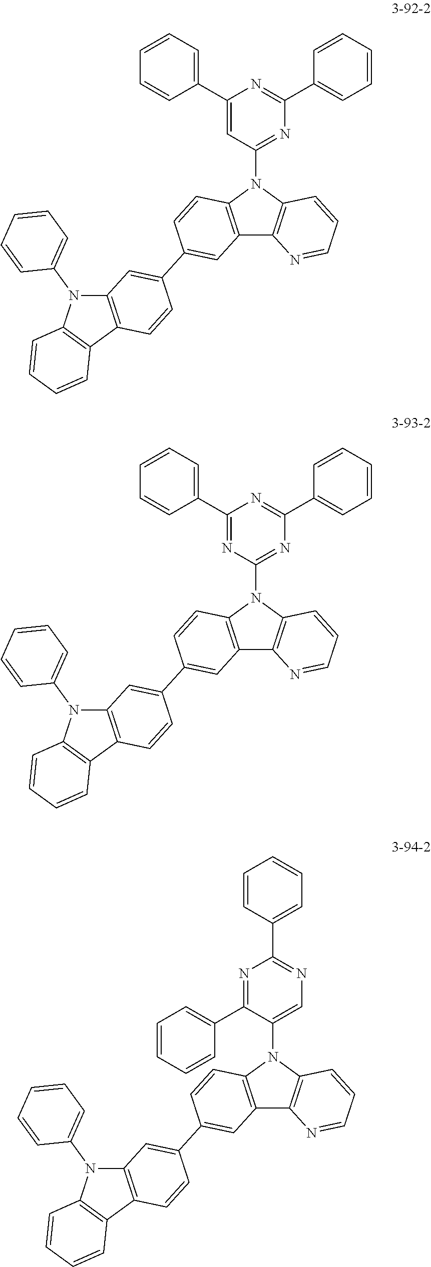 [SMILES: c1ccc(-c2cc(-n3c4ccc(-c5ccc6c7ccccc7n(-c7ccccc7)c6c5)cc4c4ncccc43)nc(-c3ccccc3)n2)cc1 ; c1ccc(-c2nc(-c3ccccc3)nc(-n3c4ccc(-c5ccc6c7ccccc7n(-c7ccccc7)c6c5)cc4c4ncccc43)n2)cc1 ; c1ccc(-c2ncc(-n3c4ccc(-c5ccc6c7ccccc7n(-c7ccccc7)c6c5)cc4c4ncccc43)c(-c3ccccc3)n2)cc1]

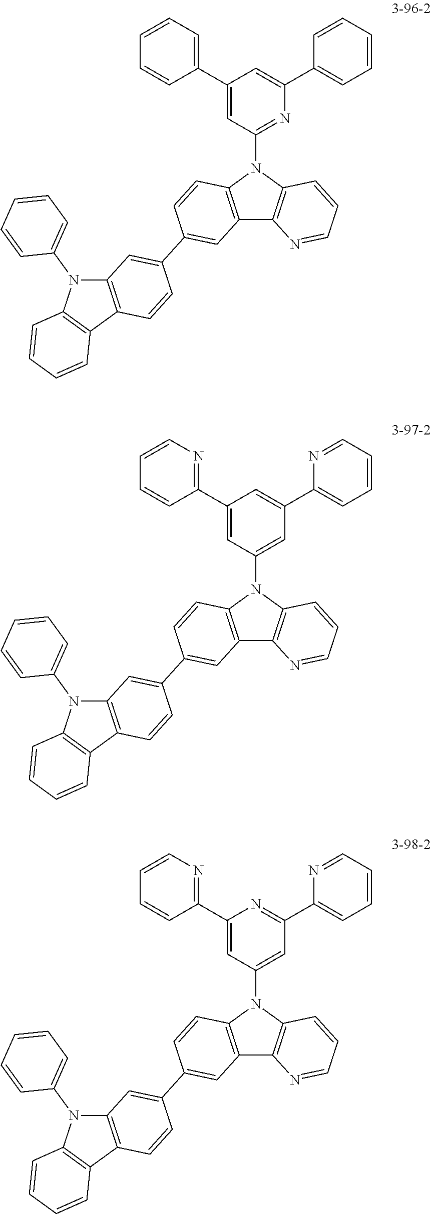 [SMILES: c1ccc(-c2cc(-c3ccccc3)nc(-n3c4ccc(-c5ccc6c7ccccc7n(-c7ccccc7)c6c5)cc4c4ncccc43)c2)cc1 ; c1ccc(-n2c3ccccc3c3ccc(-c4ccc5c(c4)c4ncccc4n5-c4cc(-c5ccccn5)cc(-c5ccccn5)c4)cc32)cc1 ; c1ccc(-n2c3ccccc3c3ccc(-c4ccc5c(c4)c4ncccc4n5-c4cc(-c5ccccn5)nc(-c5ccccn5)c4)cc32)cc1]

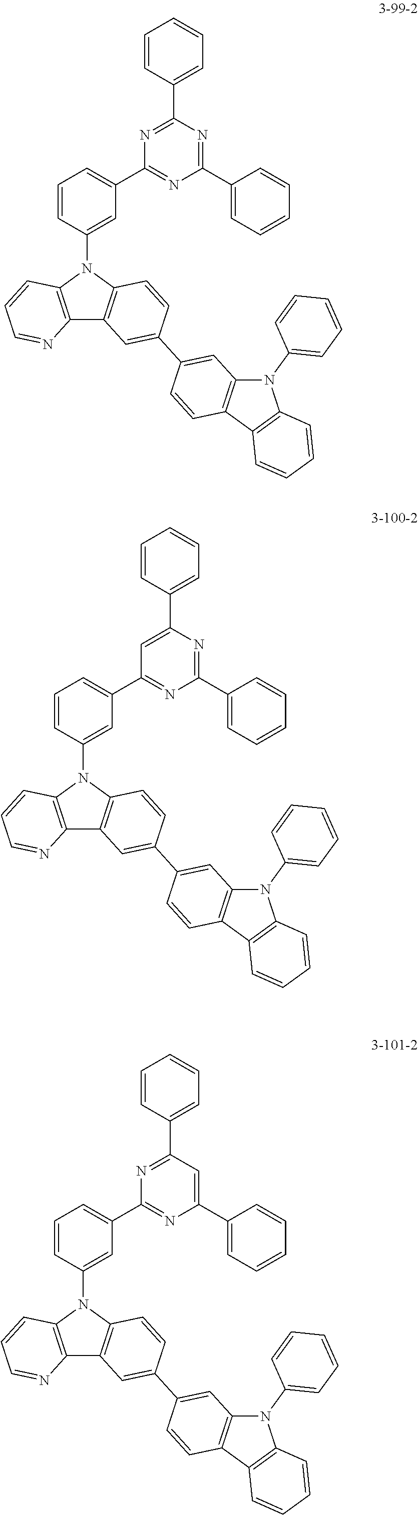 [SMILES: c1ccc(-c2nc(-c3ccccc3)nc(-c3cccc(-n4c5ccc(-c6ccc7c8ccccc8n(-c8ccccc8)c7c6)cc5c5ncccc54)c3)n2)cc1 ; c1ccc(-c2cc(-c3cccc(-n4c5ccc(-c6ccc7c8ccccc8n(-c8ccccc8)c7c6)cc5c5ncccc54)c3)nc(-c3ccccc3)n2)cc1 ; c1ccc(-c2cc(-c3ccccc3)nc(-c3cccc(-n4c5ccc(-c6ccc7c8ccccc8n(-c8ccccc8)c7c6)cc5c5ncccc54)c3)n2)cc1]

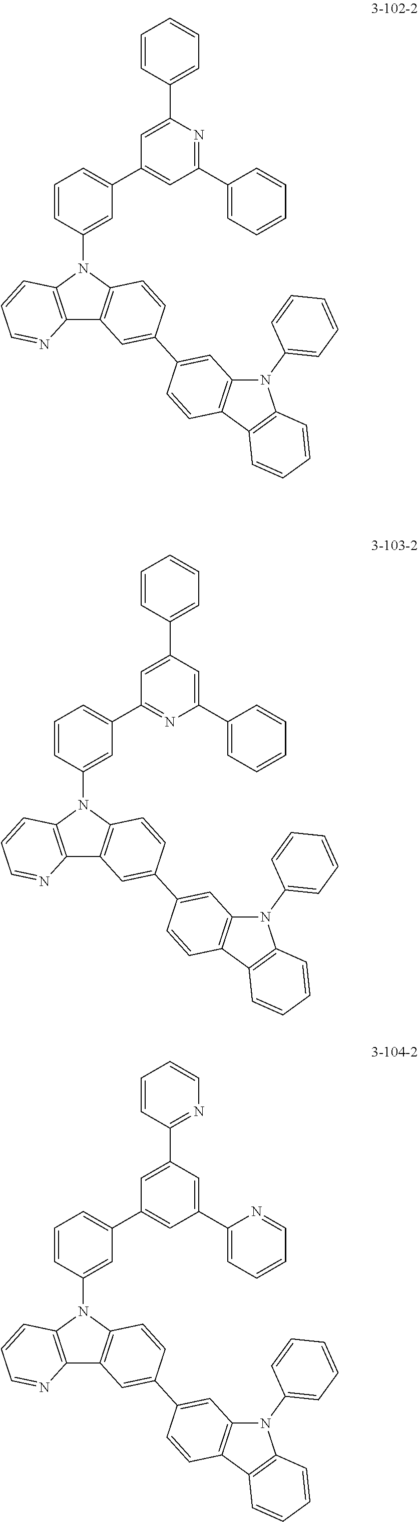 [SMILES: c1ccc(-c2cc(-c3cccc(-n4c5ccc(-c6ccc7c8ccccc8n(-c8ccccc8)c7c6)cc5c5ncccc54)c3)cc(-c3ccccc3)n2)cc1 ; c1ccc(-c2cc(-c3ccccc3)nc(-c3cccc(-n4c5ccc(-c6ccc7c8ccccc8n(-c8ccccc8)c7c6)cc5c5ncccc54)c3)c2)cc1 ; c1ccc(-n2c3ccccc3c3ccc(-c4ccc5c(c4)c4ncccc4n5-c4cccc(-c5cc(-c6ccccn6)cc(-c6ccccn6)c5)c4)cc32)cc1]

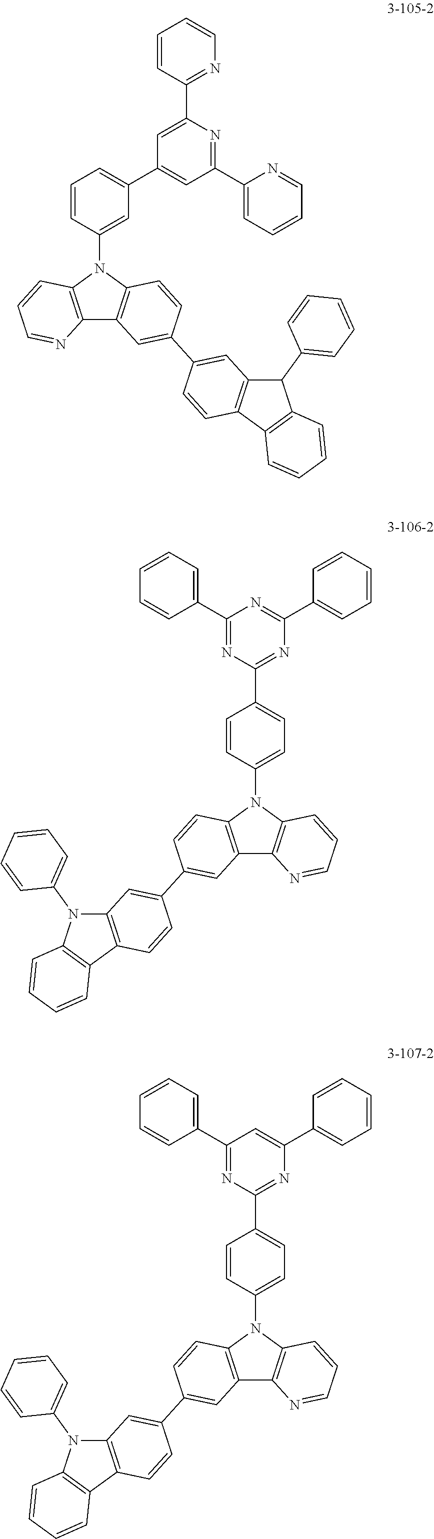 [SMILES: c1ccc(C2c3ccccc3-c3ccc(-c4ccc5c(c4)c4ncccc4n5-c4cccc(-c5cc(-c6ccccn6)nc(-c6ccccn6)c5)c4)cc32)cc1 ; c1ccc(-c2nc(-c3ccccc3)nc(-c3ccc(-n4c5ccc(-c6ccc7c8ccccc8n(-c8ccccc8)c7c6)cc5c5ncccc54)cc3)n2)cc1 ; c1ccc(-c2cc(-c3ccccc3)nc(-c3ccc(-n4c5ccc(-c6ccc7c8ccccc8n(-c8ccccc8)c7c6)cc5c5ncccc54)cc3)n2)cc1]

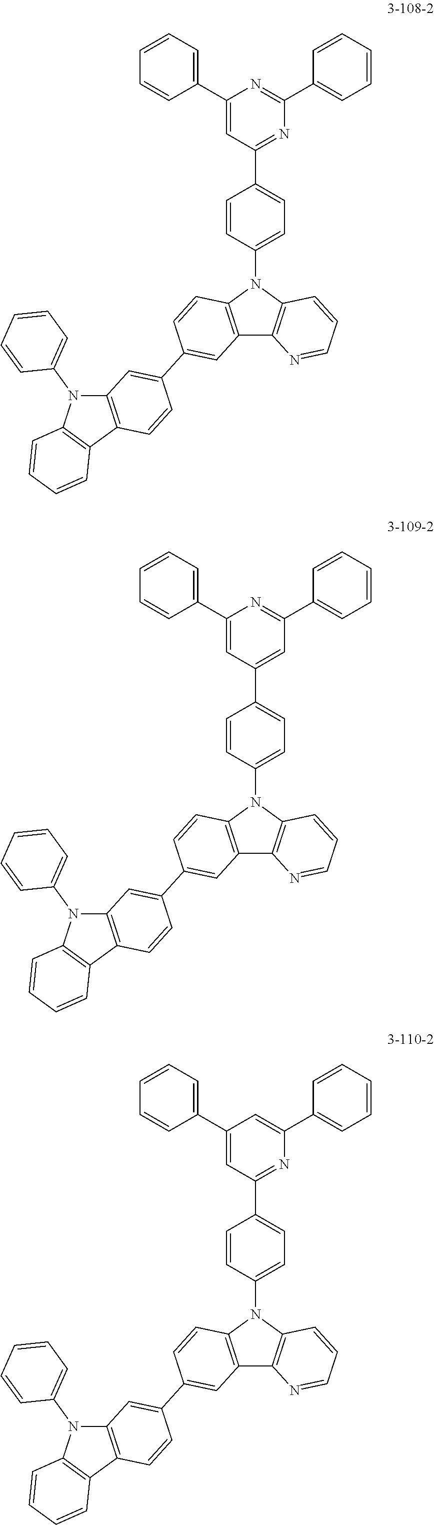 [SMILES: c1ccc(-c2cc(-c3ccc(-n4c5ccc(-c6ccc7c8ccccc8n(-c8ccccc8)c7c6)cc5c5ncccc54)cc3)nc(-c3ccccc3)n2)cc1 ; c1ccc(-c2cc(-c3ccc(-n4c5ccc(-c6ccc7c8ccccc8n(-c8ccccc8)c7c6)cc5c5ncccc54)cc3)cc(-c3ccccc3)n2)cc1 ; c1ccc(-c2cc(-c3ccccc3)nc(-c3ccc(-n4c5ccc(-c6ccc7c8ccccc8n(-c8ccccc8)c7c6)cc5c5ncccc54)cc3)c2)cc1]

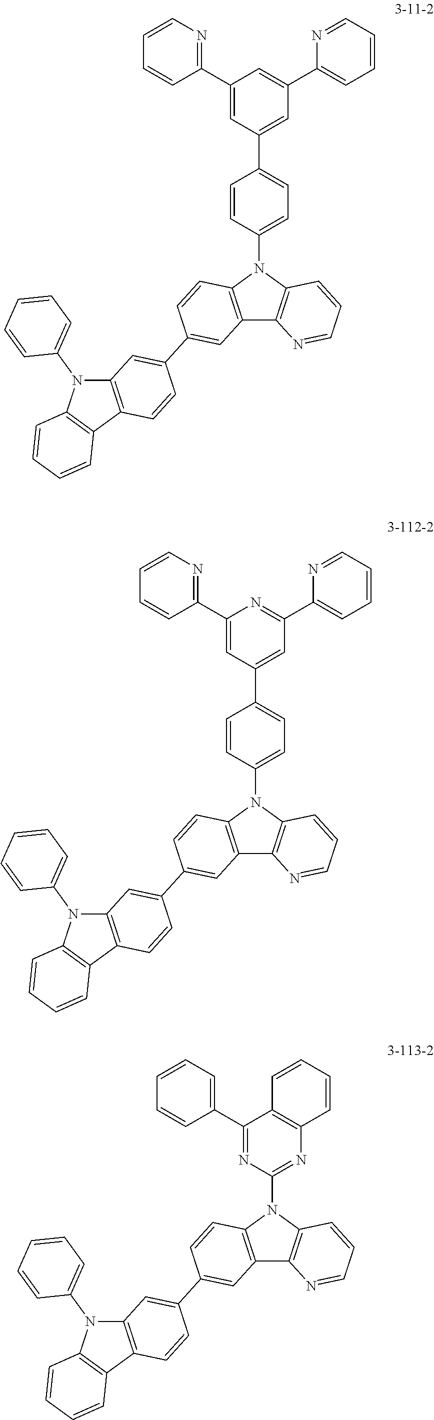 [SMILES: c1ccc(-n2c3ccccc3c3ccc(-c4ccc5c(c4)c4ncccc4n5-c4ccc(-c5cc(-c6ccccn6)cc(-c6ccccn6)c5)cc4)cc32)cc1 ; c1ccc(-n2c3ccccc3c3ccc(-c4ccc5c(c4)c4ncccc4n5-c4ccc(-c5cc(-c6ccccn6)nc(-c6ccccn6)c5)cc4)cc32)cc1 ; c1ccc(-c2nc(-n3c4ccc(-c5ccc6c7ccccc7n(-c7ccccc7)c6c5)cc4c4ncccc43)nc3ccccc23)cc1]

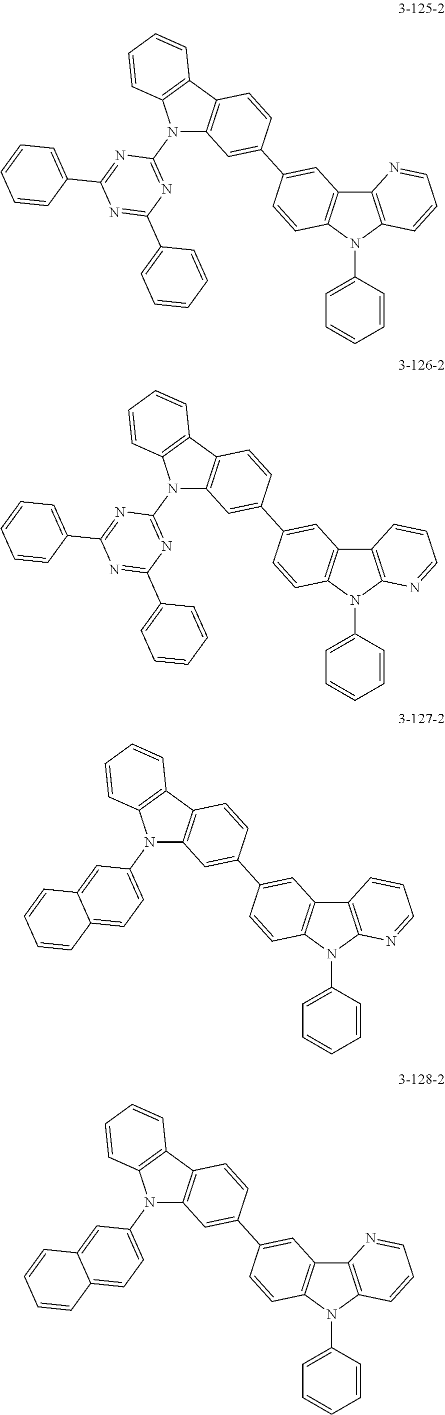 [SMILES: c1ccc(-c2nc(-c3ccccc3)nc(-n3c4ccccc4c4ccc(-c5ccc6c(c5)c5ncccc5n6-c5ccccc5)cc43)n2)cc1 ; c1ccc(-c2nc(-c3ccccc3)nc(-n3c4ccccc4c4ccc(-c5ccc6c(c5)c5cccnc5n6-c5ccccc5)cc43)n2)cc1 ; c1ccc(-n2c3ccc(-c4ccc5c6ccccc6n(-c6ccc7ccccc7c6)c5c4)cc3c3cccnc32)cc1 ; c1ccc(-n2c3ccc(-c4ccc5c6ccccc6n(-c6ccc7ccccc7c6)c5c4)cc3c3ncccc32)cc1]

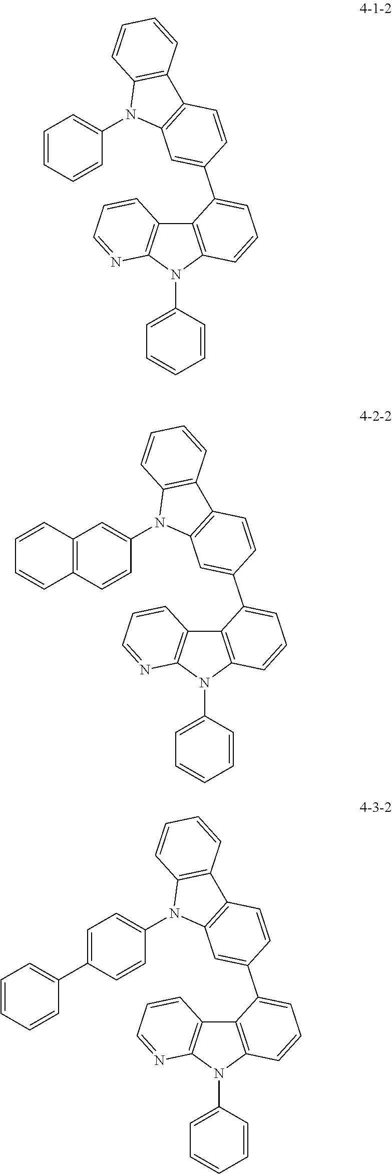

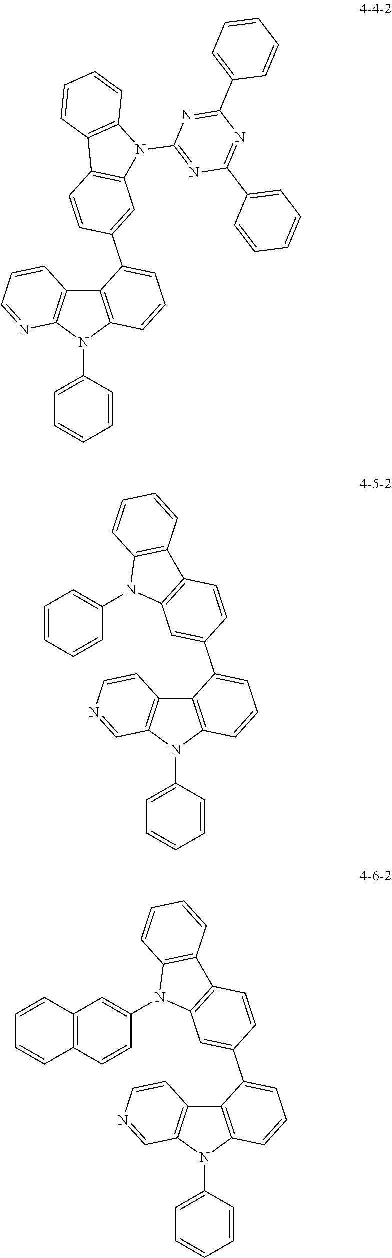 [SMILES: c1ccc(-c2nc(-c3ccccc3)nc(-n3c4ccccc4c4ccc(-c5cccc6c5c5cccnc5n6-c5ccccc5)cc43)n2)cc1 ; c1ccc(-n2c3ccccc3c3ccc(-c4cccc5c4c4ccncc4n5-c4ccccc4)cc32)cc1 ; c1ccc(-n2c3cnccc3c3c(-c4ccc5c6ccccc6n(-c6ccc7ccccc7c6)c5c4)cccc32)cc1]

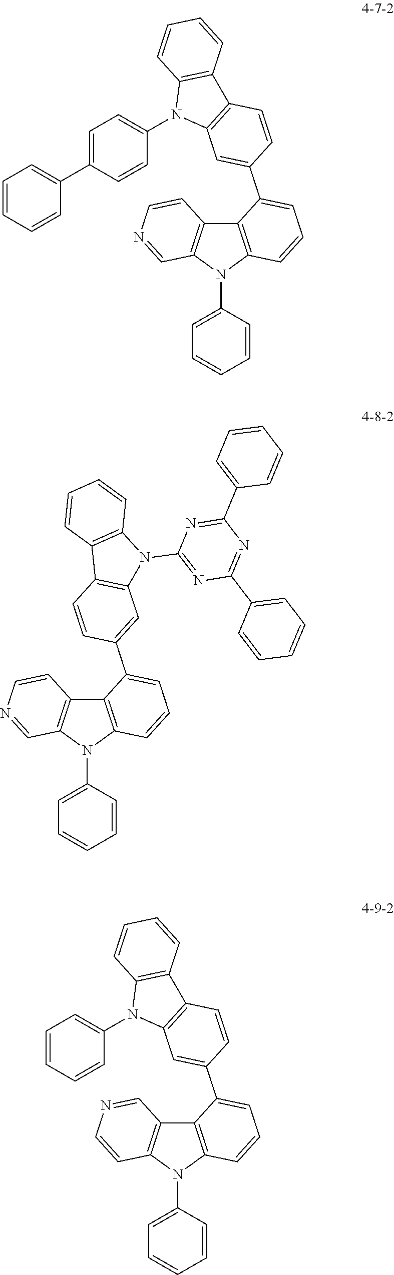 [SMILES: c1ccc(-c2ccc(-n3c4ccccc4c4ccc(-c5cccc6c5c5ccncc5n6-c5ccccc5)cc43)cc2)cc1 ; c1ccc(-c2nc(-c3ccccc3)nc(-n3c4ccccc4c4ccc(-c5cccc6c5c5ccncc5n6-c5ccccc5)cc43)n2)cc1 ; c1ccc(-n2c3ccccc3c3ccc(-c4cccc5c4c4cnccc4n5-c4ccccc4)cc32)cc1]

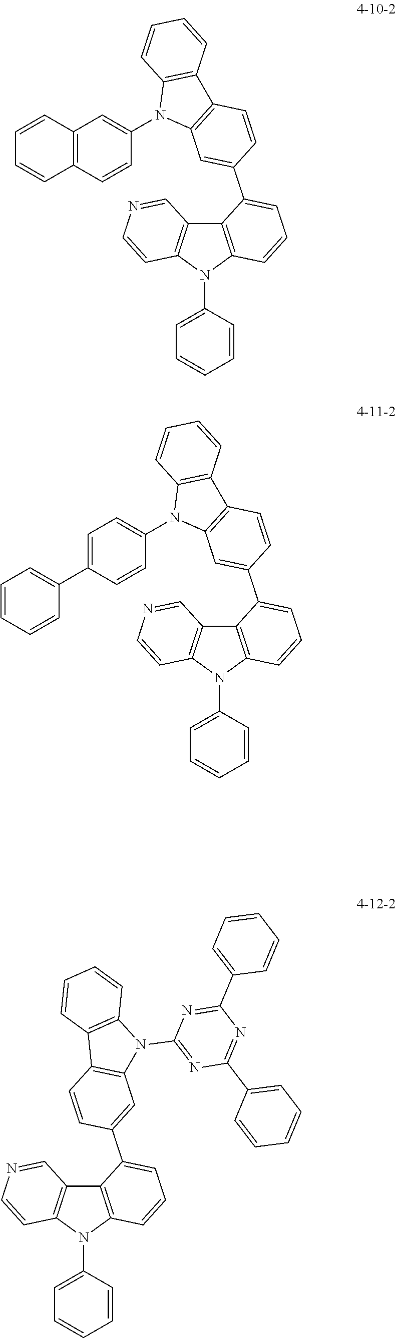

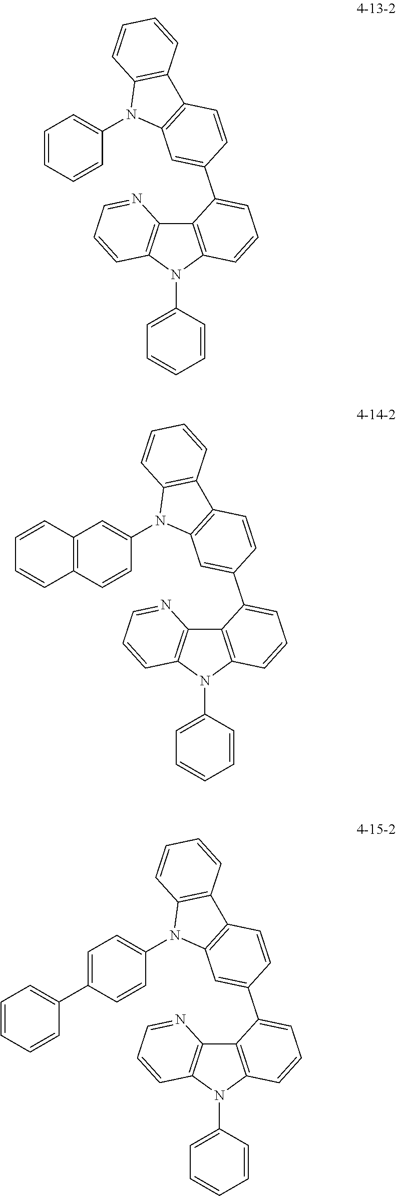 [SMILES: c1ccc(-n2c3ccccc3c3ccc(-c4cccc5c4c4ncccc4n5-c4ccccc4)cc32)cc1 ; c1ccc(-n2c3cccnc3c3c(-c4ccc5c6ccccc6n(-c6ccc7ccccc7c6)c5c4)cccc32)cc1 ; c1ccc(-c2ccc(-n3c4ccccc4c4ccc(-c5cccc6c5c5ncccc5n6-c5ccccc5)cc43)cc2)cc1]

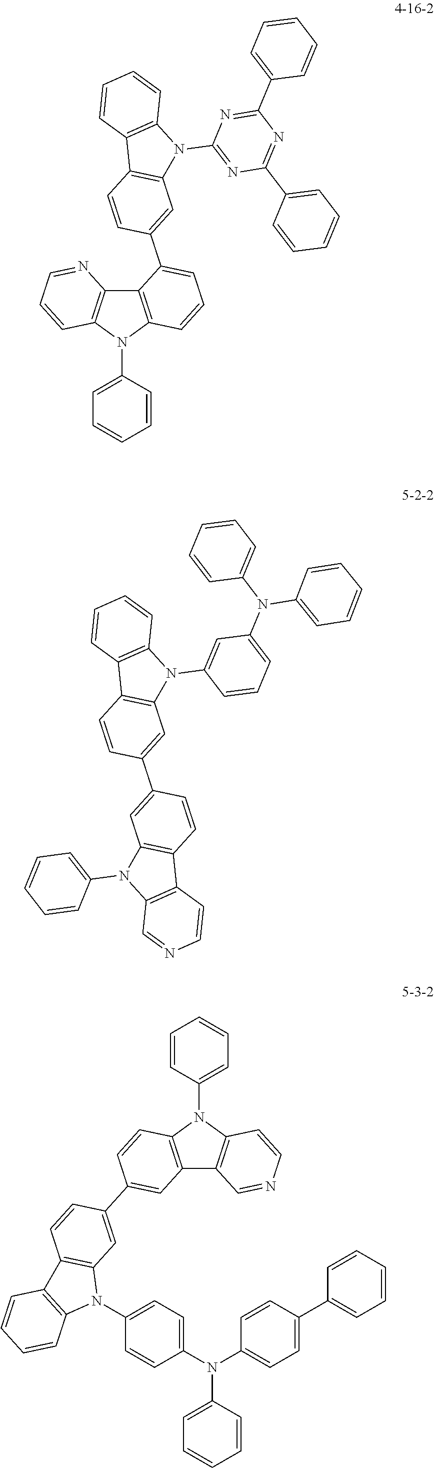 [SMILES: c1ccc(-c2nc(-c3ccccc3)nc(-n3c4ccccc4c4ccc(-c5cccc6c5c5ncccc5n6-c5ccccc5)cc43)n2)cc1 ; c1ccc(N(c2ccccc2)c2cccc(-n3c4ccccc4c4ccc(-c5ccc6c7ccncc7n(-c7ccccc7)c6c5)cc43)c2)cc1 ; c1ccc(-c2ccc(N(c3ccccc3)c3ccc(-n4c5ccccc5c5ccc(-c6ccc7c(c6)c6cnccc6n7-c6ccccc6)cc54)cc3)cc2)cc1]

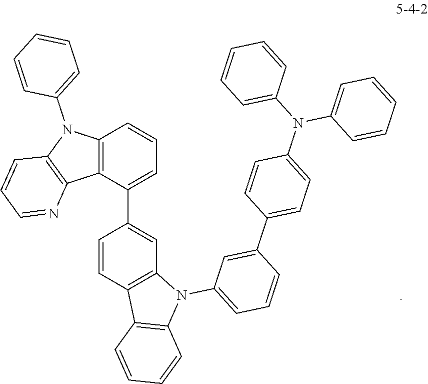 [SMILES: c1ccc(N(c2ccccc2)c2ccc(-c3cccc(-n4c5ccccc5c5ccc(-c6cccc7c6c6ncccc6n7-c6ccccc6)cc54)c3)cc2)cc1]

7. The compound of claim 1, wherein the compound is represented by one of the formulas below: 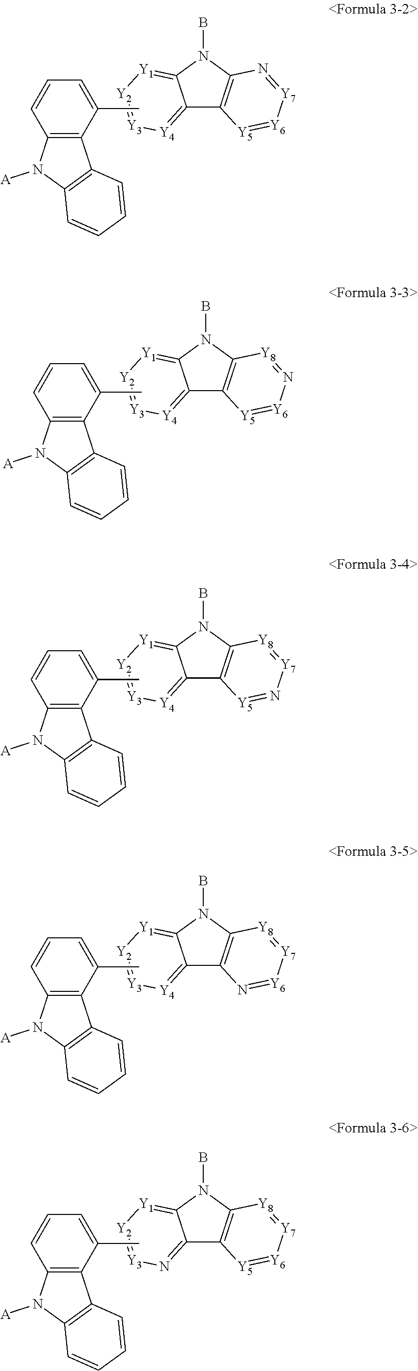 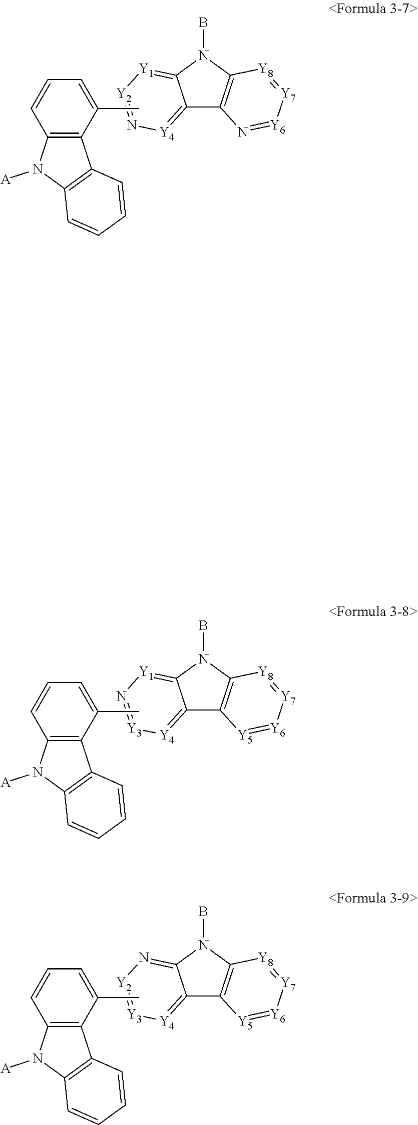
wherein in Formulas 3-2 to 3-9, Y₁ to Y₈ and A and B are identical to Y₁ to Y₈ and A and B defined in Formula 3-1.

8. The compound of claim 1, wherein the compound is represented by one of the formulas below: 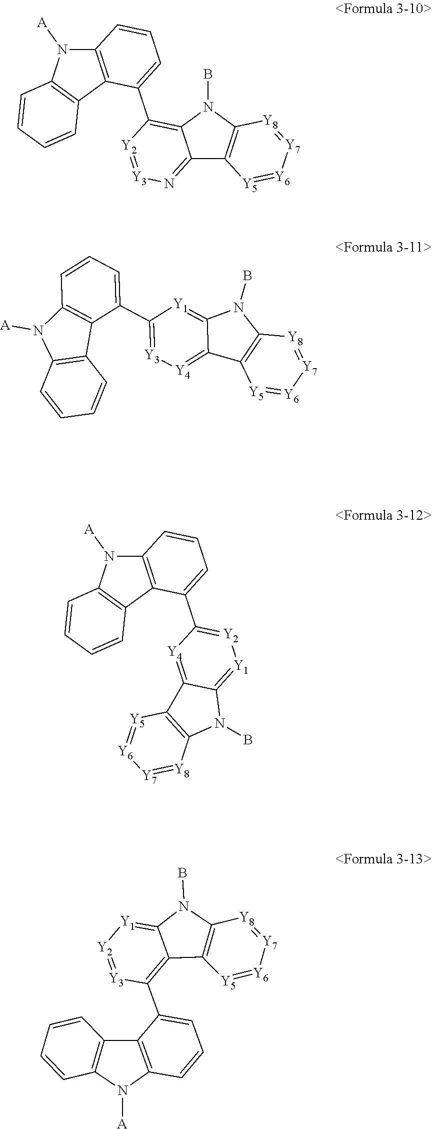
wherein in Formulas 3-10 to 3-13, Y₁ to Y₈ each are independently CH or N, and at least one of Y₁ to Y₈ is N, and A and B are identical to A and B defined in Formula 3-1.

9. The compound of claim 1, wherein the compound is represented by one of the formulas below:

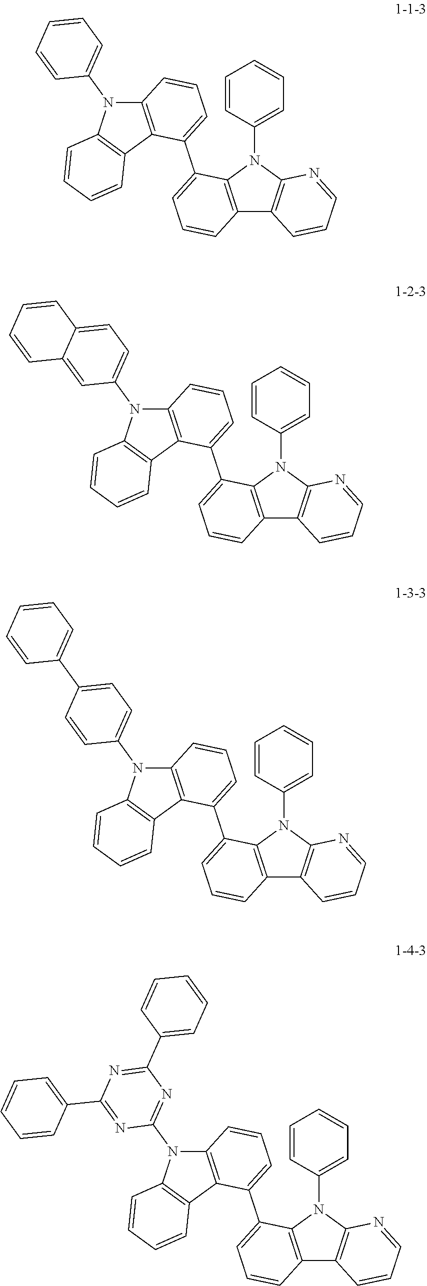 [SMILES: c1ccc(-n2c3ccccc3c3c(-c4cccc5c6cccnc6n(-c6ccccc6)c45)cccc32)cc1 ; c1ccc(-n2c3ncccc3c3cccc(-c4cccc5c4c4ccccc4n5-c4ccc5ccccc5c4)c32)cc1 ; c1ccc(-c2ccc(-n3c4ccccc4c4c(-c5cccc6c7cccnc7n(-c7ccccc7)c56)cccc43)cc2)cc1 ; c1ccc(-c2nc(-c3ccccc3)nc(-n3c4ccccc4c4c(-c5cccc6c7cccnc7n(-c7ccccc7)c56)cccc43)n2)cc1]

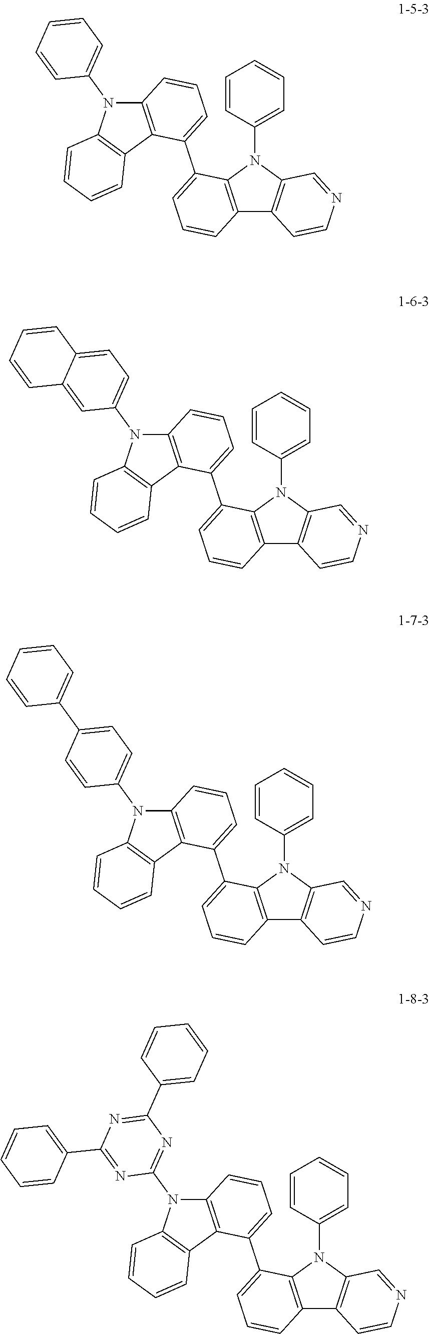 [SMILES: c1ccc(-n2c3ccccc3c3c(-c4cccc5c6ccncc6n(-c6ccccc6)c45)cccc32)cc1 ; c1ccc(-n2c3cnccc3c3cccc(-c4cccc5c4c4ccccc4n5-c4ccc5ccccc5c4)c32)cc1 ; c1ccc(-c2ccc(-n3c4ccccc4c4c(-c5cccc6c7ccncc7n(-c7ccccc7)c56)cccc43)cc2)cc1 ; c1ccc(-c2nc(-c3ccccc3)nc(-n3c4ccccc4c4c(-c5cccc6c7ccncc7n(-c7ccccc7)c56)cccc43)n2)cc1]

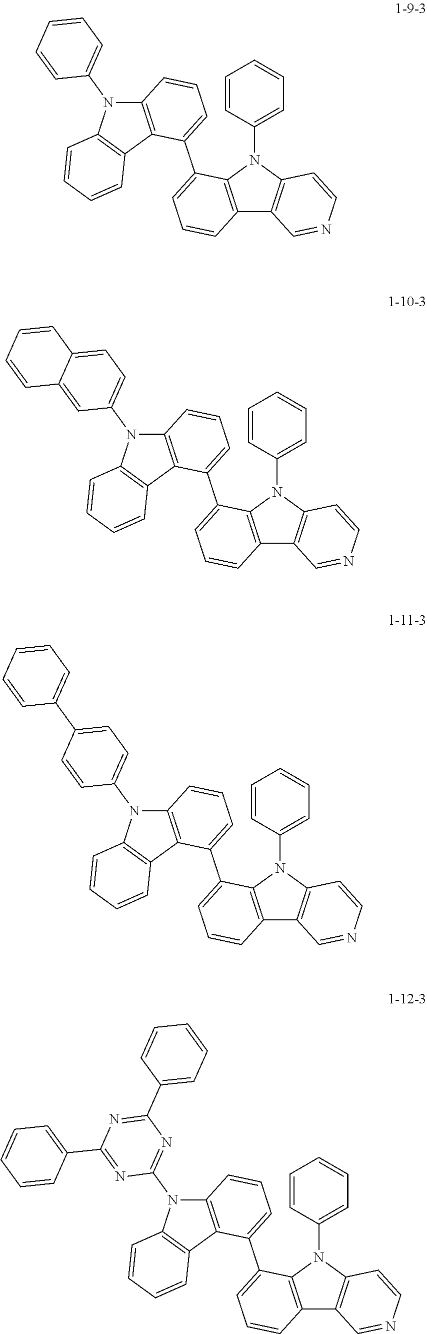 [SMILES: c1ccc(-n2c3ccccc3c3c(-c4cccc5c6cnccc6n(-c6ccccc6)c45)cccc32)cc1 ; c1ccc(-n2c3ccncc3c3cccc(-c4cccc5c4c4ccccc4n5-c4ccc5ccccc5c4)c32)cc1 ; c1ccc(-c2ccc(-n3c4ccccc4c4c(-c5cccc6c7cnccc7n(-c7ccccc7)c56)cccc43)cc2)cc1 ; c1ccc(-c2nc(-c3ccccc3)nc(-n3c4ccccc4c4c(-c5cccc6c7cnccc7n(-c7ccccc7)c56)cccc43)n2)cc1]

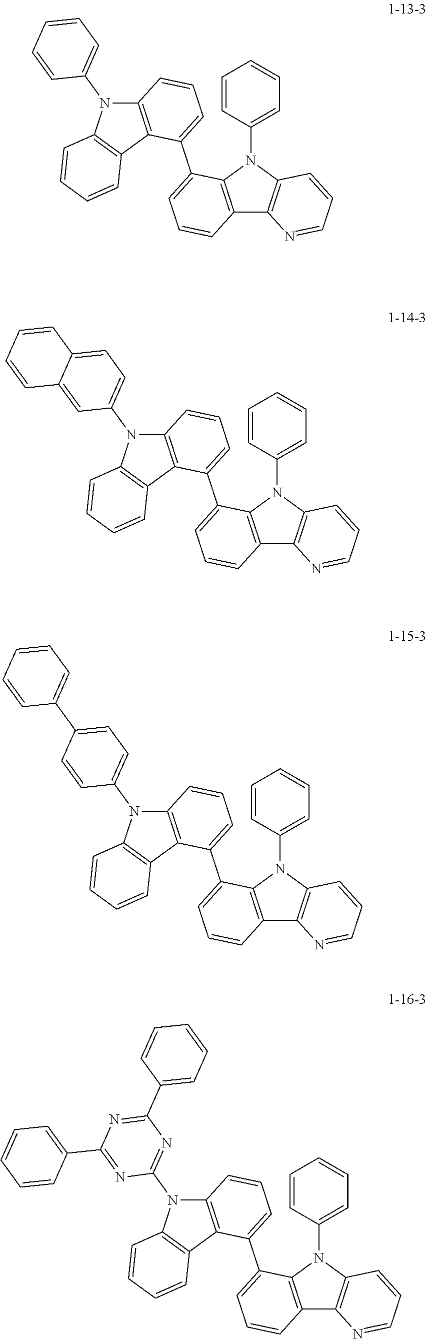 [SMILES: c1ccc(-n2c3ccccc3c3c(-c4cccc5c6ncccc6n(-c6ccccc6)c45)cccc32)cc1 ; c1ccc(-n2c3cccnc3c3cccc(-c4cccc5c4c4ccccc4n5-c4ccc5ccccc5c4)c32)cc1 ; c1ccc(-c2ccc(-n3c4ccccc4c4c(-c5cccc6c7ncccc7n(-c7ccccc7)c56)cccc43)cc2)cc1 ; c1ccc(-c2nc(-c3ccccc3)nc(-n3c4ccccc4c4c(-c5cccc6c7ncccc7n(-c7ccccc7)c56)cccc43)n2)cc1]

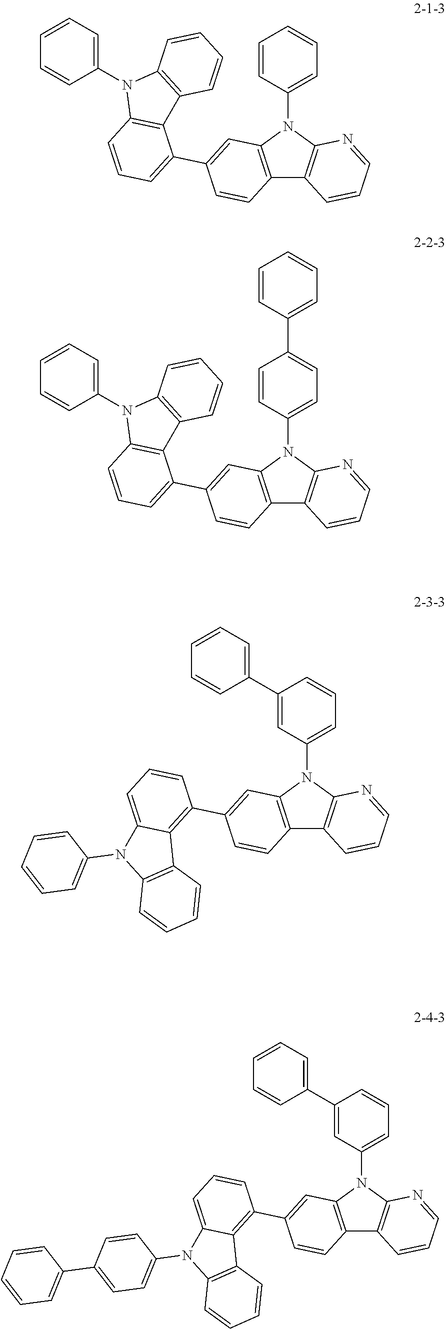 [SMILES: c1ccc(-n2c3ccccc3c3c(-c4ccc5c6cccnc6n(-c6ccccc6)c5c4)cccc32)cc1 ; c1ccc(-c2ccc(-n3c4cc(-c5cccc6c5c5ccccc5n6-c5ccccc5)ccc4c4cccnc43)cc2)cc1 ; c1ccc(-c2cccc(-n3c4cc(-c5cccc6c5c5ccccc5n6-c5ccccc5)ccc4c4cccnc43)c2)cc1 ; c1ccc(-c2ccc(-n3c4ccccc4c4c(-c5ccc6c7cccnc7n(-c7cccc(-c8ccccc8)c7)c6c5)cccc43)cc2)cc1]

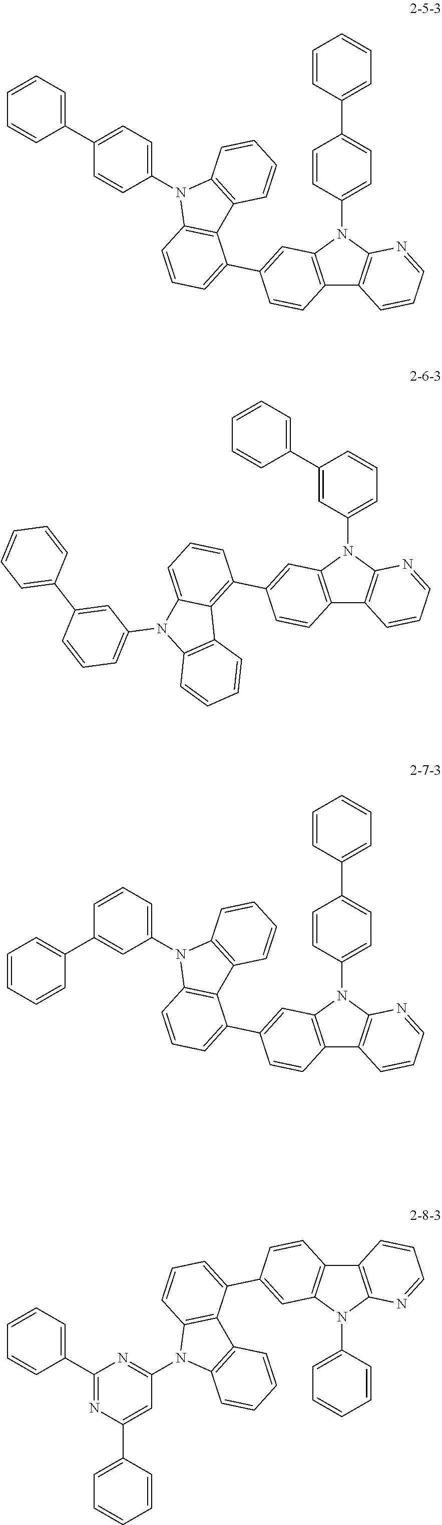 [SMILES: c1ccc(-c2ccc(-n3c4ccccc4c4c(-c5ccc6c7cccnc7n(-c7ccc(-c8ccccc8)cc7)c6c5)cccc43)cc2)cc1 ; c1ccc(-c2cccc(-n3c4ccccc4c4c(-c5ccc6c7cccnc7n(-c7cccc(-c8ccccc8)c7)c6c5)cccc43)c2)cc1 ; c1ccc(-c2ccc(-n3c4cc(-c5cccc6c5c5ccccc5n6-c5cccc(-c6ccccc6)c5)ccc4c4cccnc43)cc2)cc1 ; c1ccc(-c2cc(-n3c4ccccc4c4c(-c5ccc6c7cccnc7n(-c7ccccc7)c6c5)cccc43)nc(-c3ccccc3)n2)cc1]

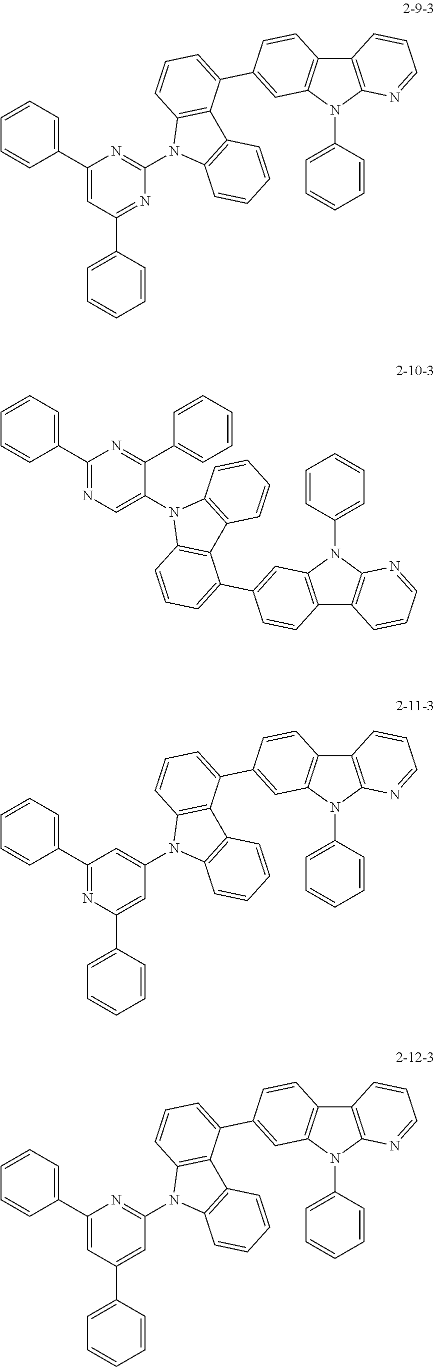 [SMILES: c1ccc(-c2cc(-c3ccccc3)nc(-n3c4ccccc4c4c(-c5ccc6c7cccnc7n(-c7ccccc7)c6c5)cccc43)n2)cc1 ; c1ccc(-c2ncc(-n3c4ccccc4c4c(-c5ccc6c7cccnc7n(-c7ccccc7)c6c5)cccc43)c(-c3ccccc3)n2)cc1 ; c1ccc(-c2cc(-n3c4ccccc4c4c(-c5ccc6c7cccnc7n(-c7ccccc7)c6c5)cccc43)cc(-c3ccccc3)n2)cc1 ; c1ccc(-c2cc(-c3ccccc3)nc(-n3c4ccccc4c4c(-c5ccc6c7cccnc7n(-c7ccccc7)c6c5)cccc43)c2)cc1]

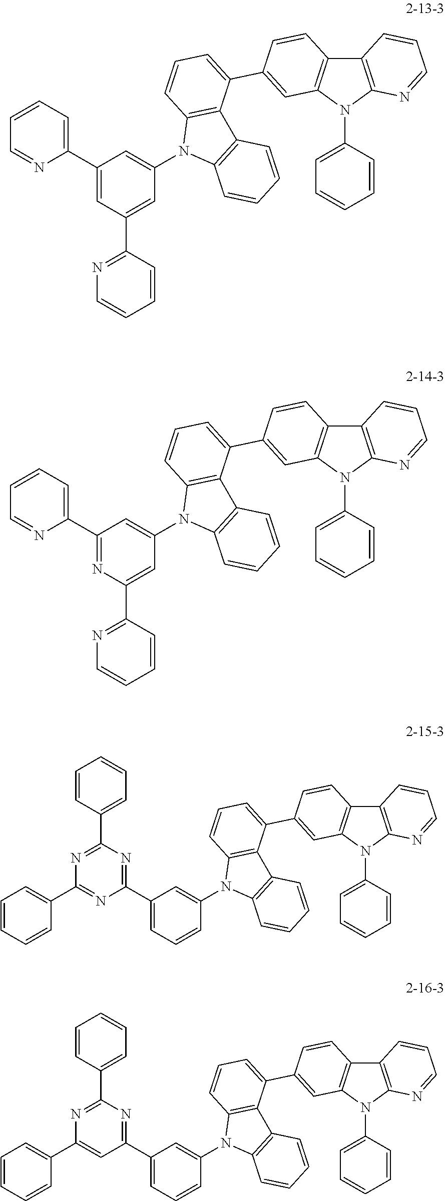 [SMILES: c1ccc(-n2c3cc(-c4cccc5c4c4ccccc4n5-c4cc(-c5ccccn5)cc(-c5ccccn5)c4)ccc3c3cccnc32)cc1 ; c1ccc(-n2c3cc(-c4cccc5c4c4ccccc4n5-c4cc(-c5ccccn5)nc(-c5ccccn5)c4)ccc3c3cccnc32)cc1 ; c1ccc(-c2nc(-c3ccccc3)nc(-c3cccc(-n4c5ccccc5c5c(-c6ccc7c8cccnc8n(-c8ccccc8)c7c6)cccc54)c3)n2)cc1 ; c1ccc(-c2cc(-c3cccc(-n4c5ccccc5c5c(-c6ccc7c8cccnc8n(-c8ccccc8)c7c6)cccc54)c3)nc(-c3ccccc3)n2)cc1]

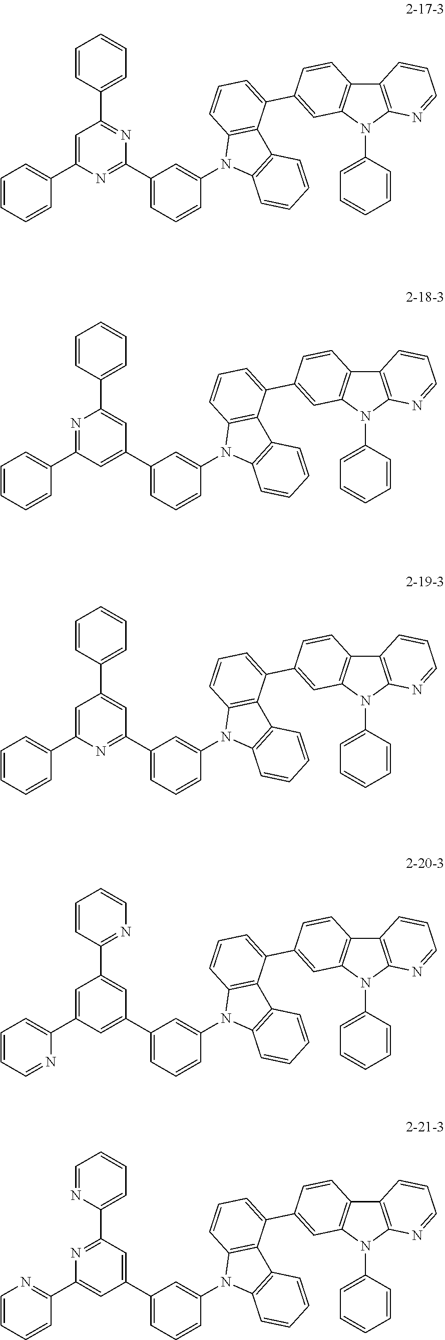 [SMILES: c1ccc(-c2cc(-c3ccccc3)nc(-c3cccc(-n4c5ccccc5c5c(-c6ccc7c8cccnc8n(-c8ccccc8)c7c6)cccc54)c3)n2)cc1 ; c1ccc(-c2cc(-c3cccc(-n4c5ccccc5c5c(-c6ccc7c8cccnc8n(-c8ccccc8)c7c6)cccc54)c3)cc(-c3ccccc3)n2)cc1 ; c1ccc(-c2cc(-c3ccccc3)nc(-c3cccc(-n4c5ccccc5c5c(-c6ccc7c8cccnc8n(-c8ccccc8)c7c6)cccc54)c3)c2)cc1 ; c1ccc(-n2c3cc(-c4cccc5c4c4ccccc4n5-c4cccc(-c5cc(-c6ccccn6)cc(-c6ccccn6)c5)c4)ccc3c3cccnc32)cc1 ; c1ccc(-n2c3cc(-c4cccc5c4c4ccccc4n5-c4cccc(-c5cc(-c6ccccn6)nc(-c6ccccn6)c5)c4)ccc3c3cccnc32)cc1]

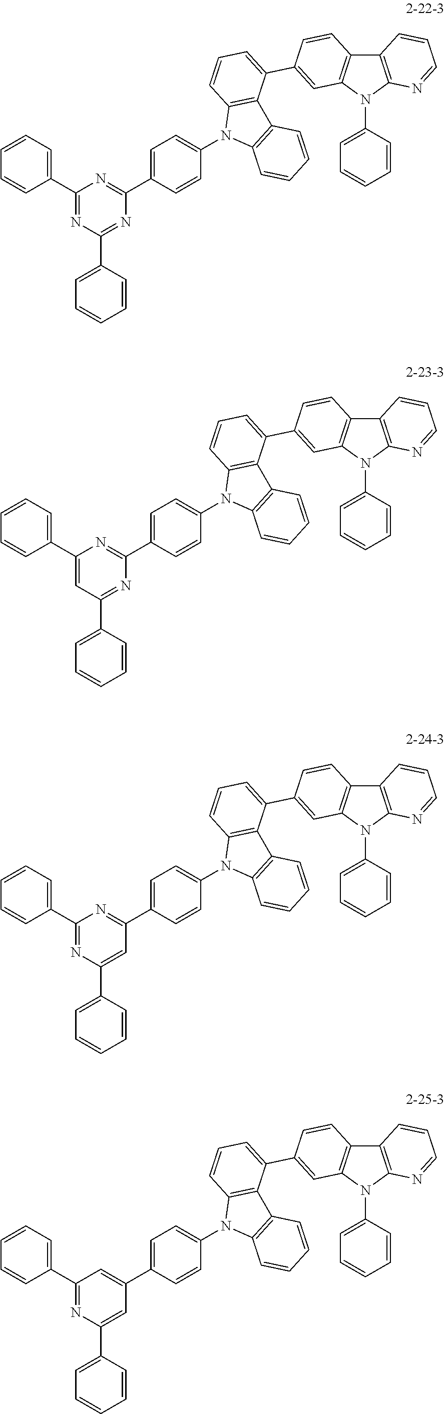 [SMILES: c1ccc(-c2nc(-c3ccccc3)nc(-c3ccc(-n4c5ccccc5c5c(-c6ccc7c8cccnc8n(-c8ccccc8)c7c6)cccc54)cc3)n2)cc1 ; c1ccc(-c2cc(-c3ccccc3)nc(-c3ccc(-n4c5ccccc5c5c(-c6ccc7c8cccnc8n(-c8ccccc8)c7c6)cccc54)cc3)n2)cc1 ; c1ccc(-c2cc(-c3ccc(-n4c5ccccc5c5c(-c6ccc7c8cccnc8n(-c8ccccc8)c7c6)cccc54)cc3)nc(-c3ccccc3)n2)cc1 ; c1ccc(-c2cc(-c3ccc(-n4c5ccccc5c5c(-c6ccc7c8cccnc8n(-c8ccccc8)c7c6)cccc54)cc3)cc(-c3ccccc3)n2)cc1]

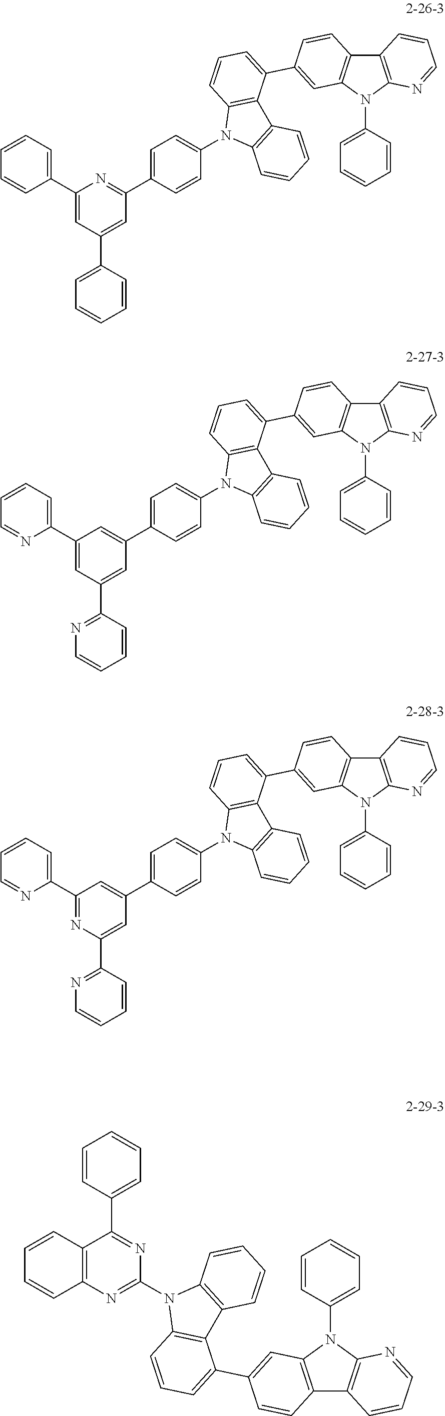 [SMILES: c1ccc(-c2cc(-c3ccccc3)nc(-c3ccc(-n4c5ccccc5c5c(-c6ccc7c8cccnc8n(-c8ccccc8)c7c6)cccc54)cc3)c2)cc1 ; c1ccc(-n2c3cc(-c4cccc5c4c4ccccc4n5-c4ccc(-c5cc(-c6ccccn6)cc(-c6ccccn6)c5)cc4)ccc3c3cccnc32)cc1 ; c1ccc(-n2c3cc(-c4cccc5c4c4ccccc4n5-c4ccc(-c5cc(-c6ccccn6)nc(-c6ccccn6)c5)cc4)ccc3c3cccnc32)cc1 ; c1ccc(-c2nc(-n3c4ccccc4c4c(-c5ccc6c7cccnc7n(-c7ccccc7)c6c5)cccc43)nc3ccccc23)cc1]

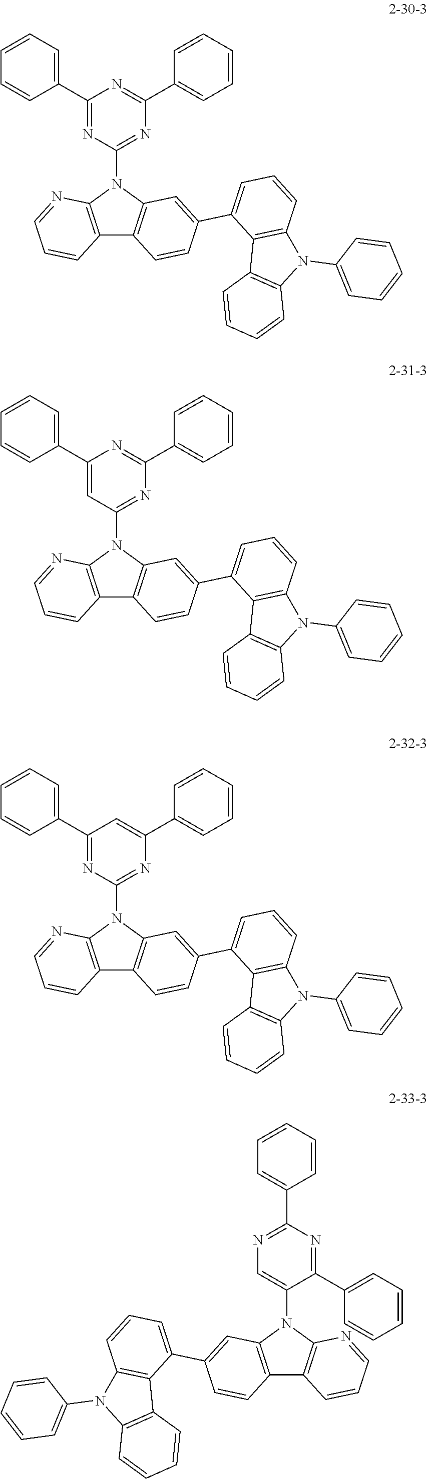 [SMILES: c1ccc(-c2nc(-c3ccccc3)nc(-n3c4cc(-c5cccc6c5c5ccccc5n6-c5ccccc5)ccc4c4cccnc43)n2)cc1 ; c1ccc(-c2cc(-n3c4cc(-c5cccc6c5c5ccccc5n6-c5ccccc5)ccc4c4cccnc43)nc(-c3ccccc3)n2)cc1 ; c1ccc(-c2cc(-c3ccccc3)nc(-n3c4cc(-c5cccc6c5c5ccccc5n6-c5ccccc5)ccc4c4cccnc43)n2)cc1 ; c1ccc(-c2ncc(-n3c4cc(-c5cccc6c5c5ccccc5n6-c5ccccc5)ccc4c4cccnc43)c(-c3ccccc3)n2)cc1]

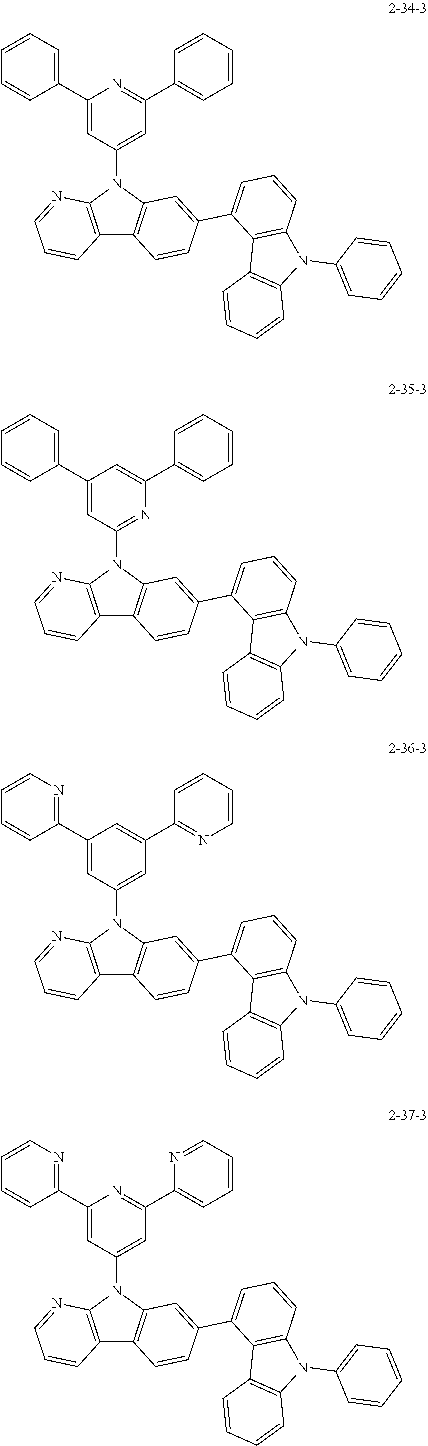 [SMILES: c1ccc(-c2cc(-n3c4cc(-c5cccc6c5c5ccccc5n6-c5ccccc5)ccc4c4cccnc43)cc(-c3ccccc3)n2)cc1 ; c1ccc(-c2cc(-c3ccccc3)nc(-n3c4cc(-c5cccc6c5c5ccccc5n6-c5ccccc5)ccc4c4cccnc43)c2)cc1 ; c1ccc(-n2c3ccccc3c3c(-c4ccc5c6cccnc6n(-c6cc(-c7ccccn7)cc(-c7ccccn7)c6)c5c4)cccc32)cc1 ; c1ccc(-n2c3ccccc3c3c(-c4ccc5c6cccnc6n(-c6cc(-c7ccccn7)nc(-c7ccccn7)c6)c5c4)cccc32)cc1]

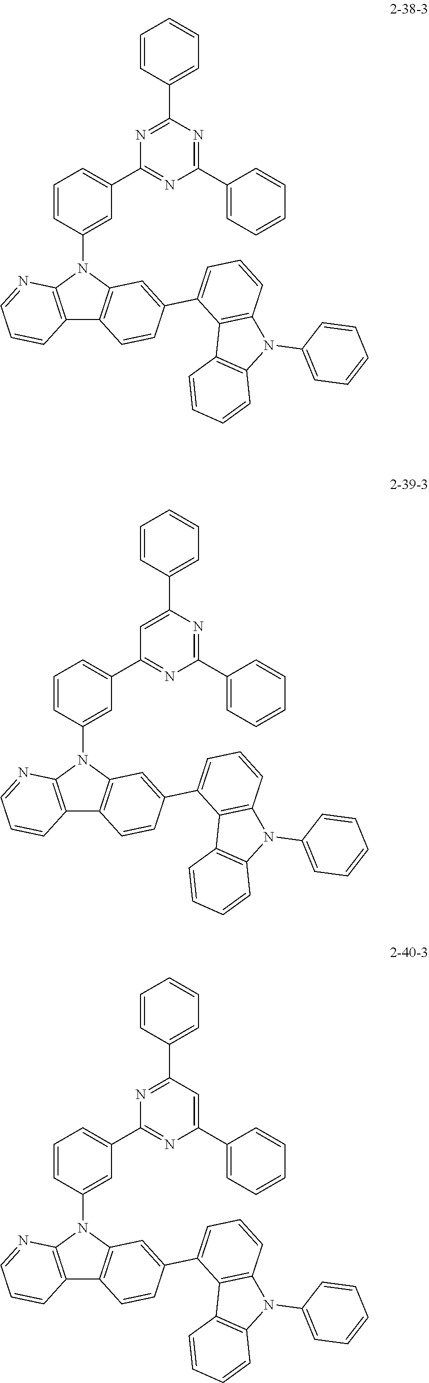 [SMILES: c1ccc(-c2nc(-c3ccccc3)nc(-c3cccc(-n4c5cc(-c6cccc7c6c6ccccc6n7-c6ccccc6)ccc5c5cccnc54)c3)n2)cc1 ; c1ccc(-c2cc(-c3cccc(-n4c5cc(-c6cccc7c6c6ccccc6n7-c6ccccc6)ccc5c5cccnc54)c3)nc(-c3ccccc3)n2)cc1 ; c1ccc(-c2cc(-c3ccccc3)nc(-c3cccc(-n4c5cc(-c6cccc7c6c6ccccc6n7-c6ccccc6)ccc5c5cccnc54)c3)n2)cc1]

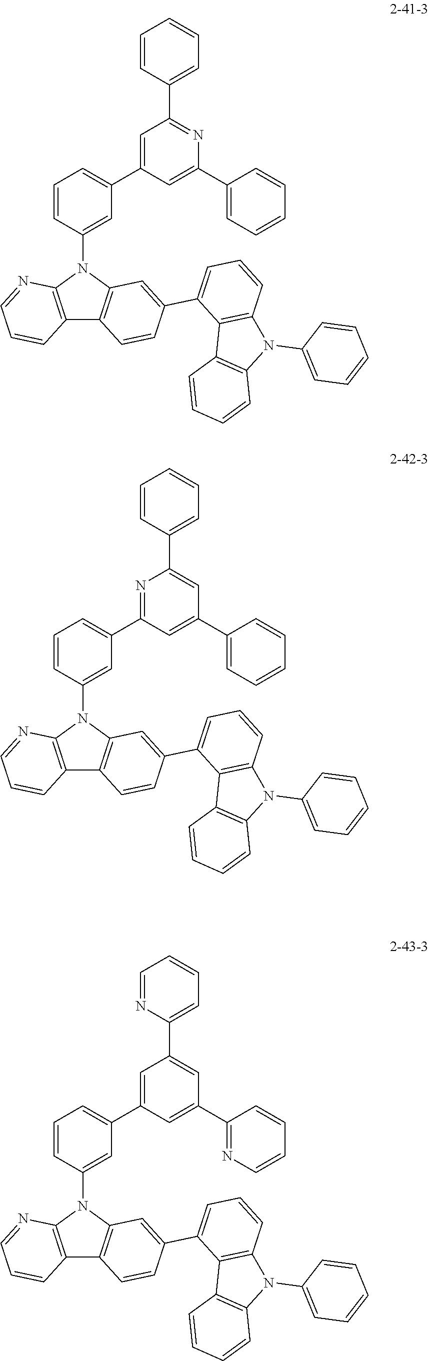 [SMILES: c1ccc(-c2cc(-c3cccc(-n4c5cc(-c6cccc7c6c6ccccc6n7-c6ccccc6)ccc5c5cccnc54)c3)cc(-c3ccccc3)n2)cc1 ; c1ccc(-c2cc(-c3ccccc3)nc(-c3cccc(-n4c5cc(-c6cccc7c6c6ccccc6n7-c6ccccc6)ccc5c5cccnc54)c3)c2)cc1 ; c1ccc(-n2c3ccccc3c3c(-c4ccc5c6cccnc6n(-c6cccc(-c7cc(-c8ccccn8)cc(-c8ccccn8)c7)c6)c5c4)cccc32)cc1]

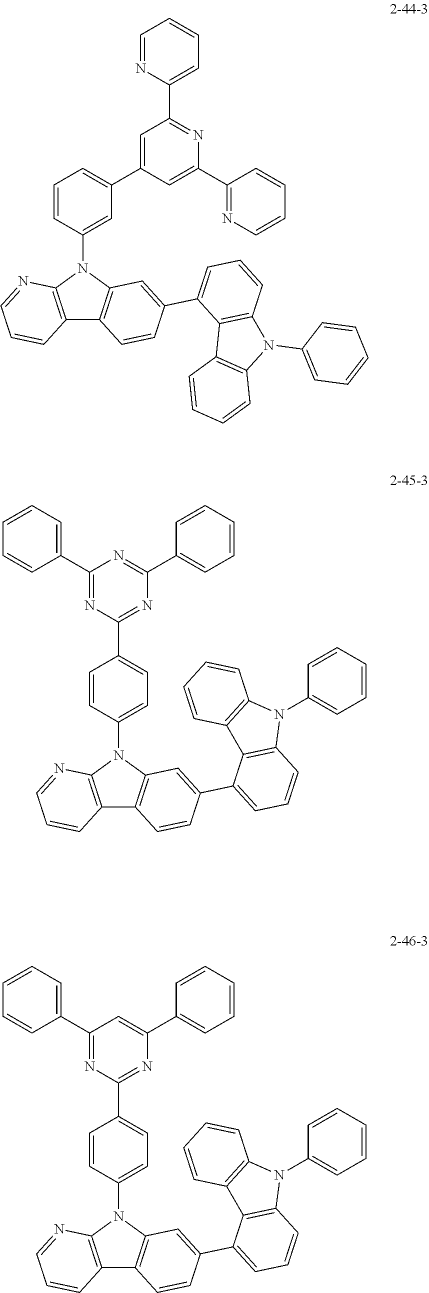 [SMILES: c1ccc(-n2c3ccccc3c3c(-c4ccc5c6cccnc6n(-c6cccc(-c7cc(-c8ccccn8)nc(-c8ccccn8)c7)c6)c5c4)cccc32)cc1 ; c1ccc(-c2nc(-c3ccccc3)nc(-c3ccc(-n4c5cc(-c6cccc7c6c6ccccc6n7-c6ccccc6)ccc5c5cccnc54)cc3)n2)cc1 ; c1ccc(-c2cc(-c3ccccc3)nc(-c3ccc(-n4c5cc(-c6cccc7c6c6ccccc6n7-c6ccccc6)ccc5c5cccnc54)cc3)n2)cc1]

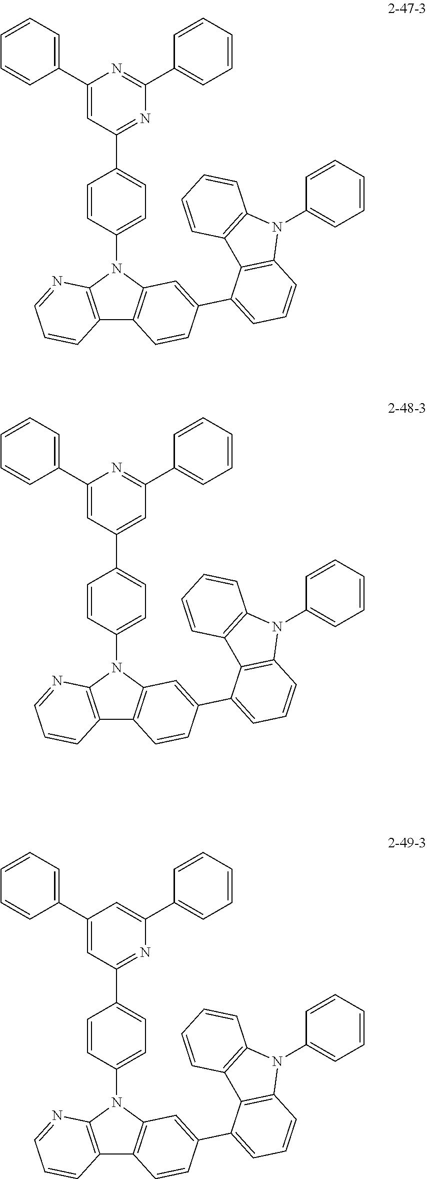 [SMILES: c1ccc(-c2cc(-c3ccc(-n4c5cc(-c6cccc7c6c6ccccc6n7-c6ccccc6)ccc5c5cccnc54)cc3)nc(-c3ccccc3)n2)cc1 ; c1ccc(-c2cc(-c3ccc(-n4c5cc(-c6cccc7c6c6ccccc6n7-c6ccccc6)ccc5c5cccnc54)cc3)cc(-c3ccccc3)n2)cc1 ; c1ccc(-c2cc(-c3ccccc3)nc(-c3ccc(-n4c5cc(-c6cccc7c6c6ccccc6n7-c6ccccc6)ccc5c5cccnc54)cc3)c2)cc1]

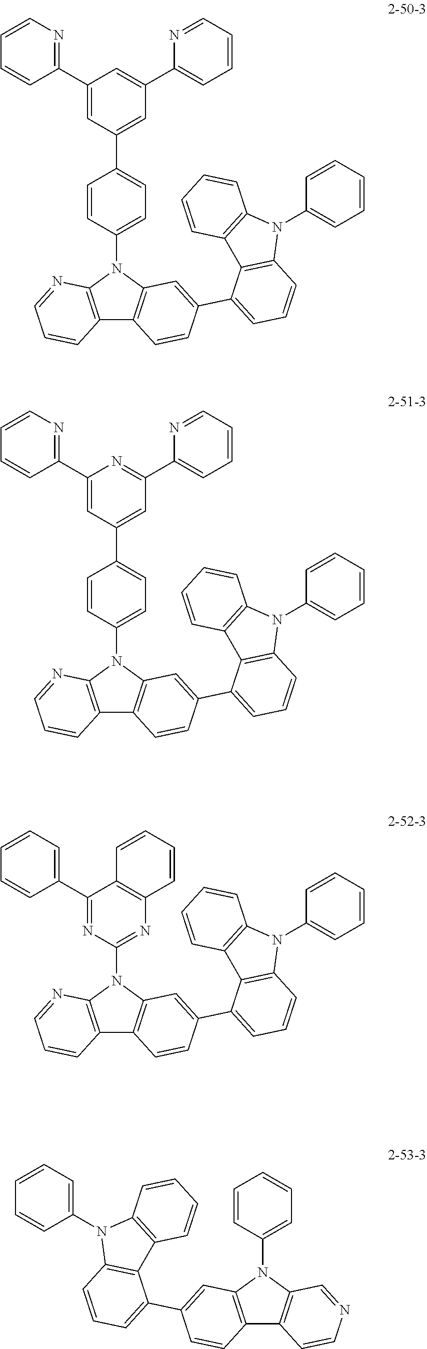 [SMILES: c1ccc(-n2c3ccccc3c3c(-c4ccc5c6cccnc6n(-c6ccc(-c7cc(-c8ccccn8)cc(-c8ccccn8)c7)cc6)c5c4)cccc32)cc1 ; c1ccc(-n2c3ccccc3c3c(-c4ccc5c6cccnc6n(-c6ccc(-c7cc(-c8ccccn8)nc(-c8ccccn8)c7)cc6)c5c4)cccc32)cc1 ; c1ccc(-c2nc(-n3c4cc(-c5cccc6c5c5ccccc5n6-c5ccccc5)ccc4c4cccnc43)nc3ccccc23)cc1 ; c1ccc(-n2c3cnccc3c3ccc(-c4cccc5c4c4ccccc4n5-c4ccccc4)cc32)cc1]

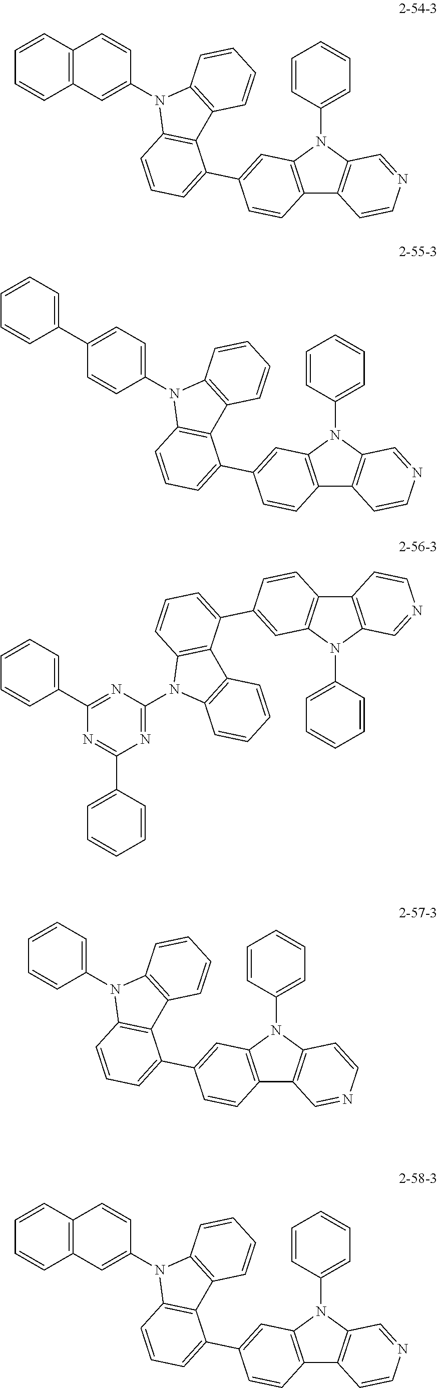 [SMILES: c1ccc(-n2c3cnccc3c3ccc(-c4cccc5c4c4ccccc4n5-c4ccc5ccccc5c4)cc32)cc1 ; c1ccc(-c2ccc(-n3c4ccccc4c4c(-c5ccc6c7ccncc7n(-c7ccccc7)c6c5)cccc43)cc2)cc1 ; c1ccc(-c2nc(-c3ccccc3)nc(-n3c4ccccc4c4c(-c5ccc6c7ccncc7n(-c7ccccc7)c6c5)cccc43)n2)cc1 ; c1ccc(-n2c3ccncc3c3ccc(-c4cccc5c4c4ccccc4n5-c4ccccc4)cc32)cc1 ; c1ccc(-n2c3cnccc3c3ccc(-c4cccc5c4c4ccccc4n5-c4ccc5ccccc5c4)cc32)cc1]

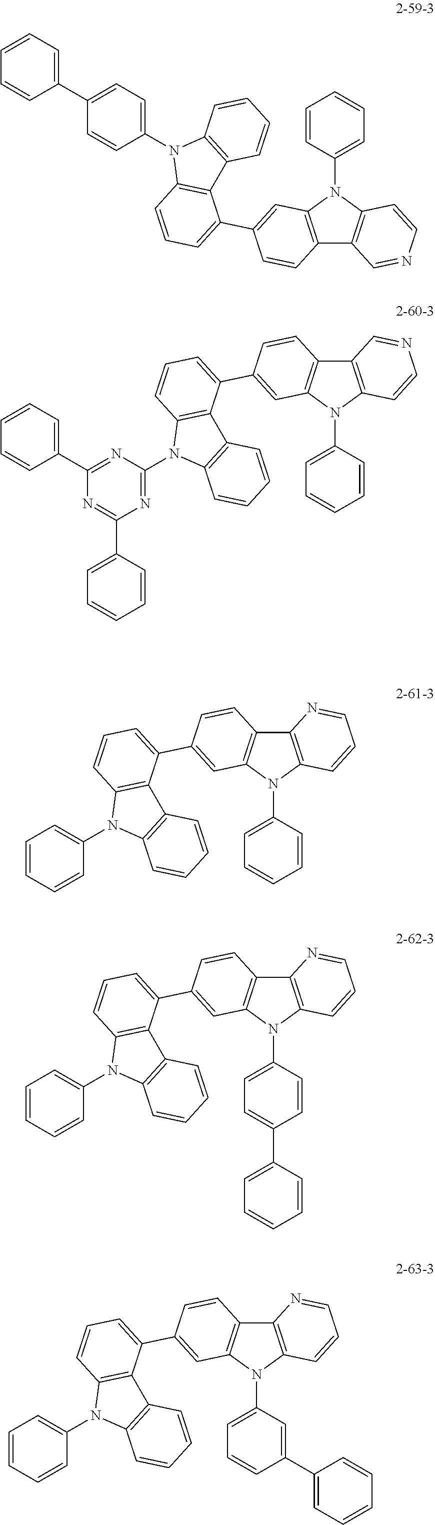 [SMILES: c1ccc(-c2ccc(-n3c4ccccc4c4c(-c5ccc6c7cnccc7n(-c7ccccc7)c6c5)cccc43)cc2)cc1 ; c1ccc(-c2nc(-c3ccccc3)nc(-n3c4ccccc4c4c(-c5ccc6c7cnccc7n(-c7ccccc7)c6c5)cccc43)n2)cc1 ; c1ccc(-n2c3cc(-c4cccc5c4c4ccccc4n5-c4ccccc4)ccc3c3ncccc32)cc1 ; c1ccc(-c2ccc(-n3c4cc(-c5cccc6c5c5ccccc5n6-c5ccccc5)ccc4c4ncccc43)cc2)cc1 ; c1ccc(-c2cccc(-n3c4cc(-c5cccc6c5c5ccccc5n6-c5ccccc5)ccc4c4ncccc43)c2)cc1]

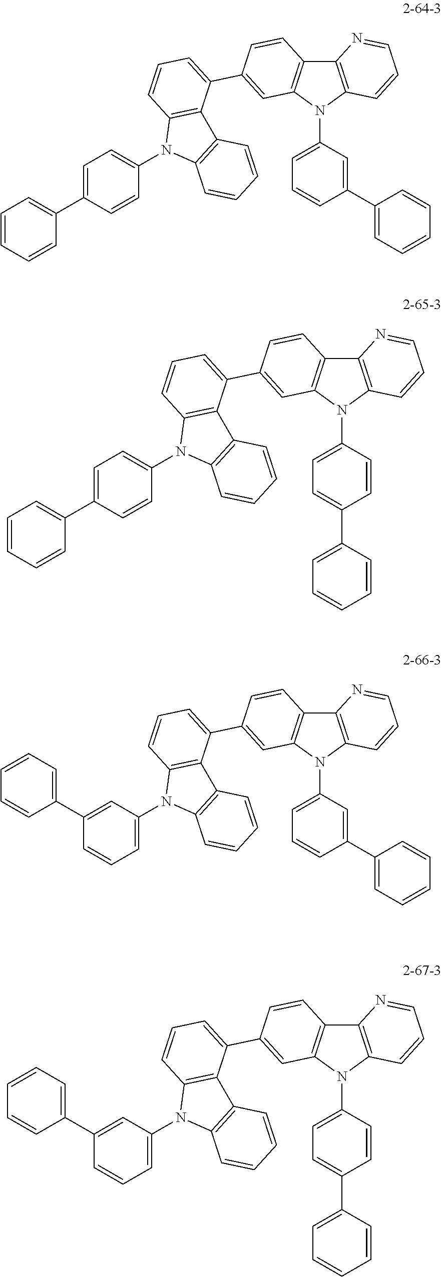 [SMILES: c1ccc(-c2ccc(-n3c4ccccc4c4c(-c5ccc6c7ncccc7n(-c7cccc(-c8ccccc8)c7)c6c5)cccc43)cc2)cc1 ; c1ccc(-c2ccc(-n3c4cc(-c5cccc6c5c5ccccc5n6-c5ccc(-c6ccccc6)cc5)ccc4c4ncccc43)cc2)cc1 ; c1ccc(-c2cccc(-n3c4cc(-c5cccc6c5c5ccccc5n6-c5cccc(-c6ccccc6)c5)ccc4c4ncccc43)c2)cc1 ; c1ccc(-c2ccc(-n3c4cc(-c5cccc6c5c5ccccc5n6-c5cccc(-c6ccccc6)c5)ccc4c4ncccc43)cc2)cc1]

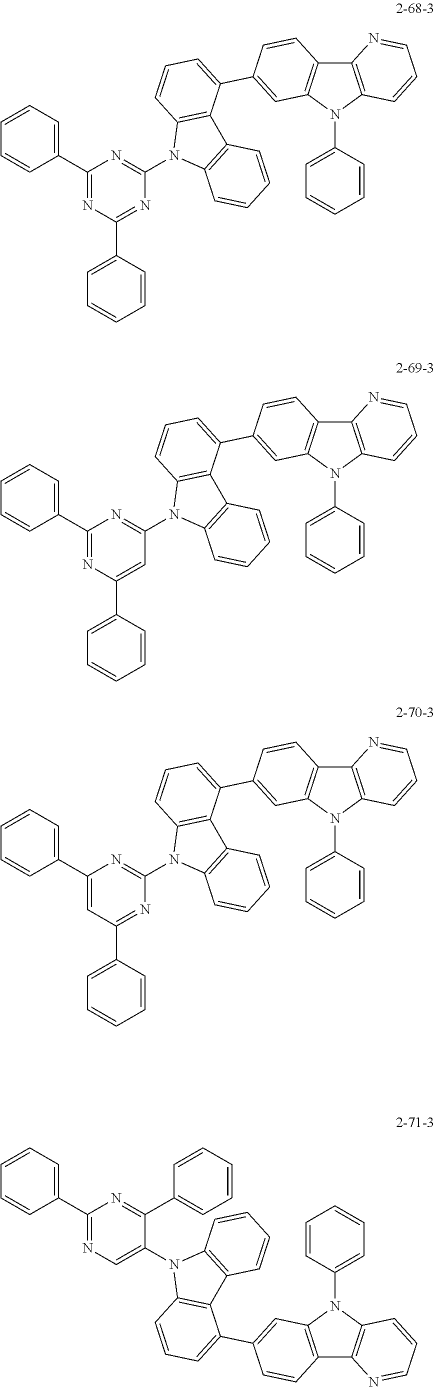 [SMILES: c1ccc(-c2nc(-c3ccccc3)nc(-n3c4ccccc4c4c(-c5ccc6c7ncccc7n(-c7ccccc7)c6c5)cccc43)n2)cc1 ; c1ccc(-c2cc(-n3c4ccccc4c4c(-c5ccc6c7ncccc7n(-c7ccccc7)c6c5)cccc43)nc(-c3ccccc3)n2)cc1 ; c1ccc(-c2cc(-c3ccccc3)nc(-n3c4ccccc4c4c(-c5ccc6c7ncccc7n(-c7ccccc7)c6c5)cccc43)n2)cc1 ; c1ccc(-c2ncc(-n3c4ccccc4c4c(-c5ccc6c7ncccc7n(-c7ccccc7)c6c5)cccc43)c(-c3ccccc3)n2)cc1]

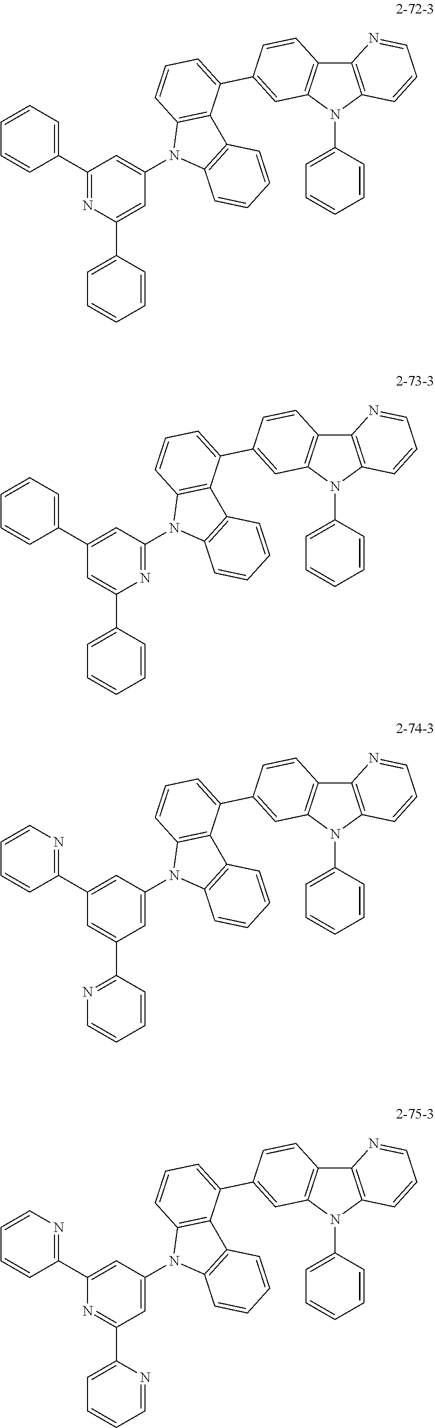 [SMILES: c1ccc(-c2cc(-n3c4ccccc4c4c(-c5ccc6c7ncccc7n(-c7ccccc7)c6c5)cccc43)cc(-c3ccccc3)n2)cc1 ; c1ccc(-c2cc(-c3ccccc3)nc(-n3c4ccccc4c4c(-c5ccc6c7ncccc7n(-c7ccccc7)c6c5)cccc43)c2)cc1 ; c1ccc(-n2c3cc(-c4cccc5c4c4ccccc4n5-c4cc(-c5ccccn5)cc(-c5ccccn5)c4)ccc3c3ncccc32)cc1 ; c1ccc(-n2c3cc(-c4cccc5c4c4ccccc4n5-c4cc(-c5ccccn5)nc(-c5ccccn5)c4)ccc3c3ncccc32)cc1]

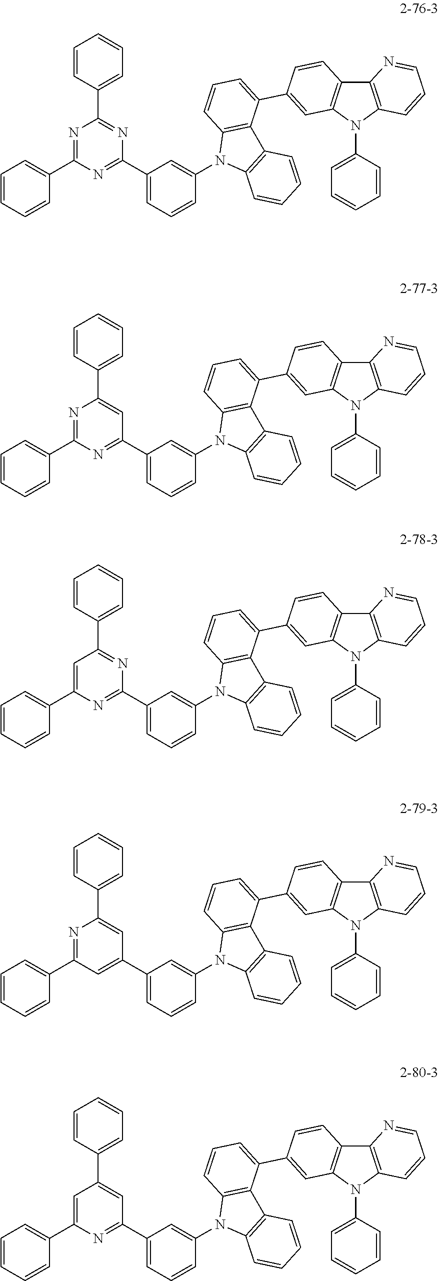 [SMILES: c1ccc(-c2nc(-c3ccccc3)nc(-c3cccc(-n4c5ccccc5c5c(-c6ccc7c8ncccc8n(-c8ccccc8)c7c6)cccc54)c3)n2)cc1 ; c1ccc(-c2cc(-c3cccc(-n4c5ccccc5c5c(-c6ccc7c8ncccc8n(-c8ccccc8)c7c6)cccc54)c3)nc(-c3ccccc3)n2)cc1 ; c1ccc(-c2cc(-c3ccccc3)nc(-c3cccc(-n4c5ccccc5c5c(-c6ccc7c8ncccc8n(-c8ccccc8)c7c6)cccc54)c3)n2)cc1 ; c1ccc(-c2cc(-c3cccc(-n4c5ccccc5c5c(-c6ccc7c8ncccc8n(-c8ccccc8)c7c6)cccc54)c3)cc(-c3ccccc3)n2)cc1 ; c1ccc(-c2cc(-c3ccccc3)nc(-c3cccc(-n4c5ccccc5c5c(-c6ccc7c8ncccc8n(-c8ccccc8)c7c6)cccc54)c3)c2)cc1]

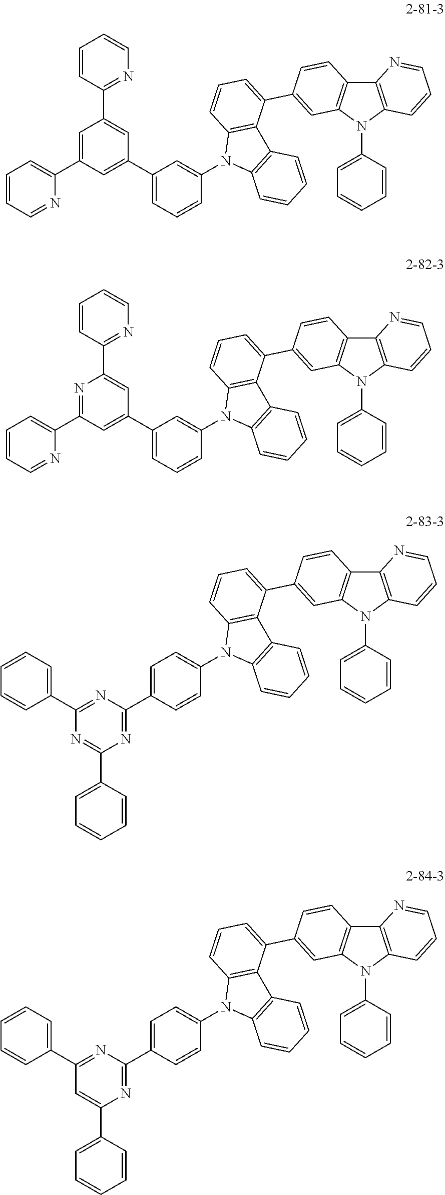 [SMILES: c1ccc(-n2c3cc(-c4cccc5c4c4ccccc4n5-c4cccc(-c5cc(-c6ccccn6)cc(-c6ccccn6)c5)c4)ccc3c3ncccc32)cc1 ; c1ccc(-n2c3cc(-c4cccc5c4c4ccccc4n5-c4cccc(-c5cc(-c6ccccn6)nc(-c6ccccn6)c5)c4)ccc3c3ncccc32)cc1 ; c1ccc(-c2nc(-c3ccccc3)nc(-c3ccc(-n4c5ccccc5c5c(-c6ccc7c8ncccc8n(-c8ccccc8)c7c6)cccc54)cc3)n2)cc1 ; c1ccc(-c2cc(-c3ccccc3)nc(-c3ccc(-n4c5ccccc5c5c(-c6ccc7c8ncccc8n(-c8ccccc8)c7c6)cccc54)cc3)n2)cc1]

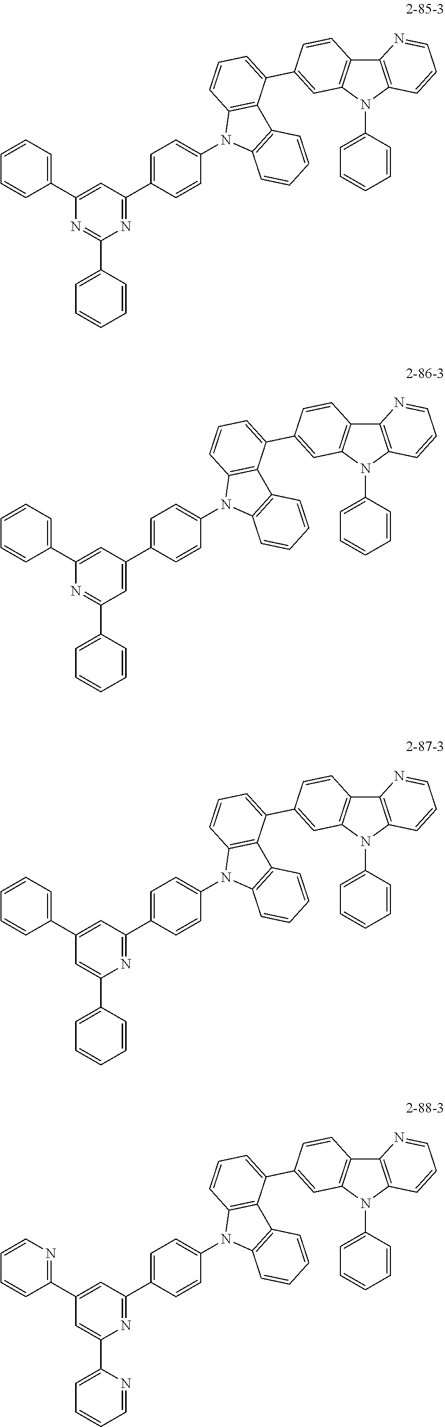 [SMILES: c1ccc(-c2cc(-c3ccc(-n4c5ccccc5c5c(-c6ccc7c8ncccc8n(-c8ccccc8)c7c6)cccc54)cc3)nc(-c3ccccc3)n2)cc1 ; c1ccc(-c2cc(-c3ccc(-n4c5ccccc5c5c(-c6ccc7c8ncccc8n(-c8ccccc8)c7c6)cccc54)cc3)cc(-c3ccccc3)n2)cc1 ; c1ccc(-c2cc(-c3ccccc3)nc(-c3ccc(-n4c5ccccc5c5c(-c6ccc7c8ncccc8n(-c8ccccc8)c7c6)cccc54)cc3)c2)cc1 ; c1ccc(-n2c3cc(-c4cccc5c4c4ccccc4n5-c4ccc(-c5cc(-c6ccccn6)cc(-c6ccccn6)n5)cc4)ccc3c3ncccc32)cc1]

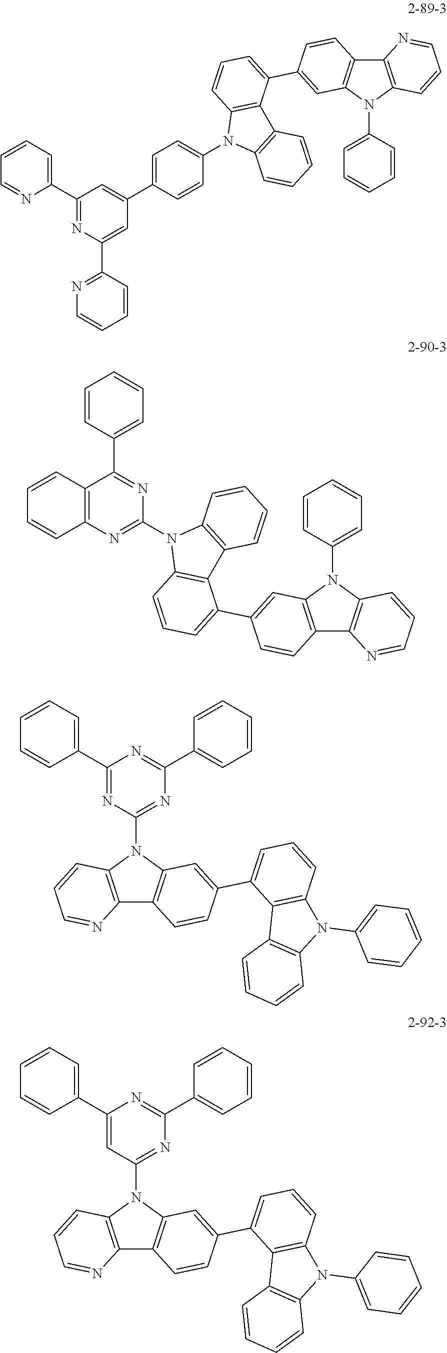

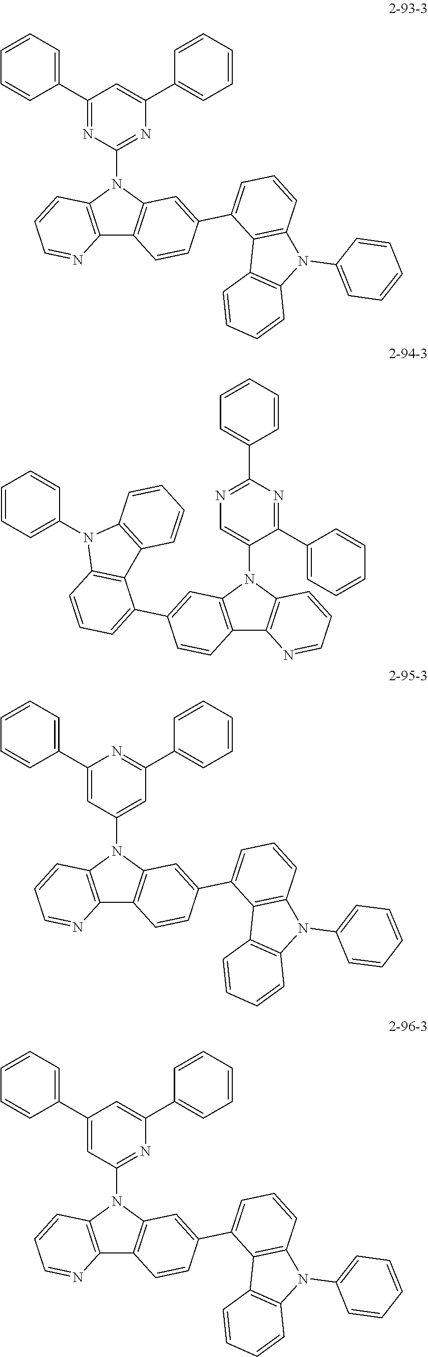

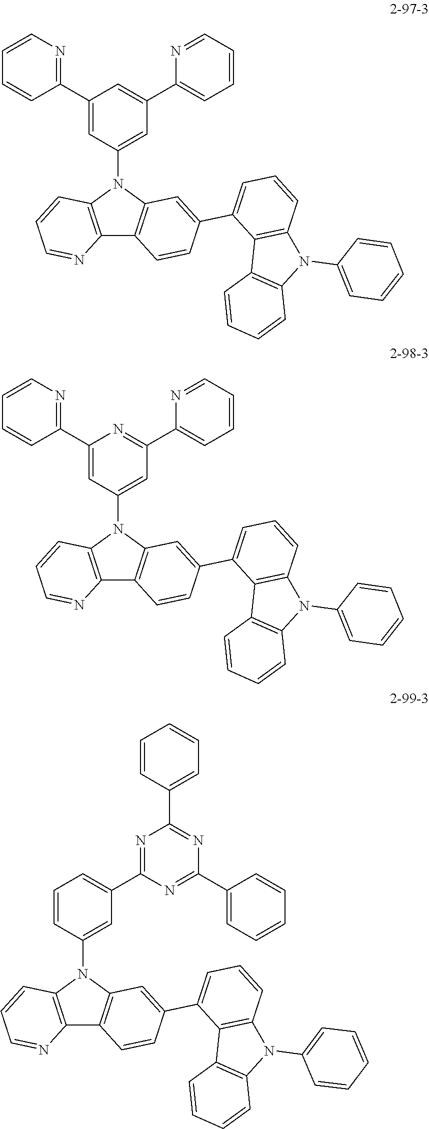 [SMILES: c1ccc(-n2c3ccccc3c3c(-c4ccc5c6ncccc6n(-c6cc(-c7ccccn7)cc(-c7ccccn7)c6)c5c4)cccc32)cc1 ; c1ccc(-n2c3ccccc3c3c(-c4ccc5c6ncccc6n(-c6cc(-c7ccccn7)nc(-c7ccccn7)c6)c5c4)cccc32)cc1 ; c1ccc(-c2nc(-c3ccccc3)nc(-c3cccc(-n4c5cc(-c6cccc7c6c6ccccc6n7-c6ccccc6)ccc5c5ncccc54)c3)n2)cc1]

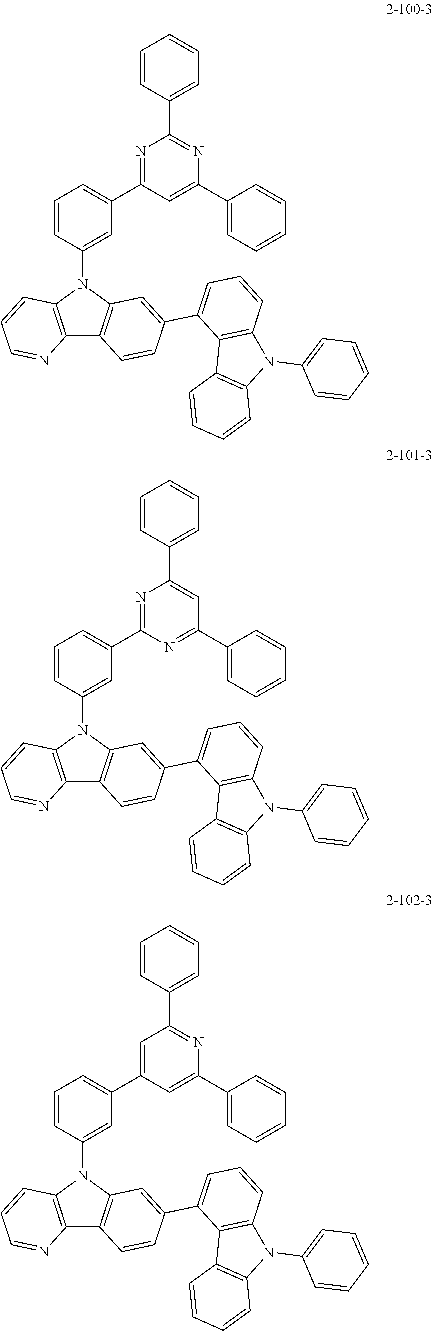 [SMILES: c1ccc(-c2cc(-c3cccc(-n4c5cc(-c6cccc7c6c6ccccc6n7-c6ccccc6)ccc5c5ncccc54)c3)nc(-c3ccccc3)n2)cc1 ; c1ccc(-c2cc(-c3ccccc3)nc(-c3cccc(-n4c5cc(-c6cccc7c6c6ccccc6n7-c6ccccc6)ccc5c5ncccc54)c3)n2)cc1 ; c1ccc(-c2cc(-c3cccc(-n4c5cc(-c6cccc7c6c6ccccc6n7-c6ccccc6)ccc5c5ncccc54)c3)cc(-c3ccccc3)n2)cc1]

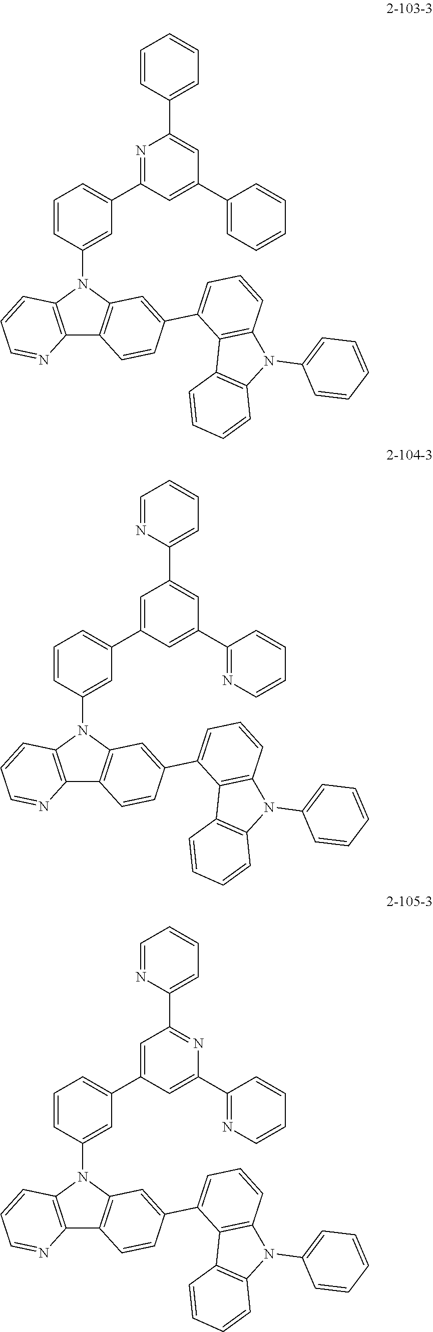 [SMILES: c1ccc(-c2cc(-c3ccccc3)nc(-c3cccc(-n4c5cc(-c6cccc7c6c6ccccc6n7-c6ccccc6)ccc5c5ncccc54)c3)c2)cc1 ; c1ccc(-n2c3ccccc3c3c(-c4ccc5c6ncccc6n(-c6cccc(-c7cc(-c8ccccn8)cc(-c8ccccn8)c7)c6)c5c4)cccc32)cc1 ; c1ccc(-n2c3ccccc3c3c(-c4ccc5c6ncccc6n(-c6cccc(-c7cc(-c8ccccn8)nc(-c8ccccn8)c7)c6)c5c4)cccc32)cc1]

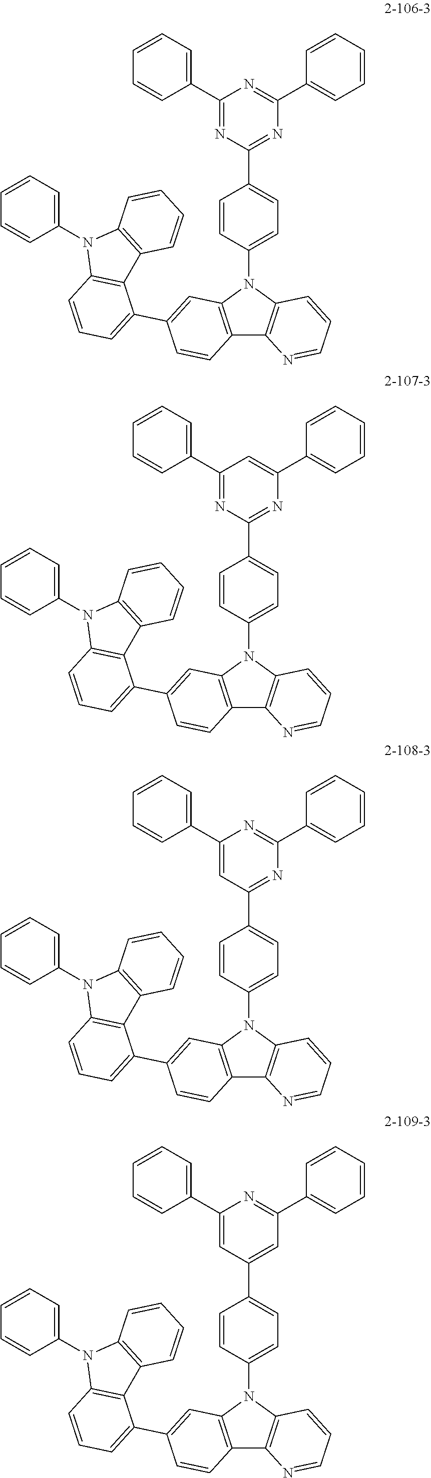 [SMILES: c1ccc(-c2nc(-c3ccccc3)nc(-c3ccc(-n4c5cc(-c6cccc7c6c6ccccc6n7-c6ccccc6)ccc5c5ncccc54)cc3)n2)cc1 ; c1ccc(-c2cc(-c3ccccc3)nc(-c3ccc(-n4c5cc(-c6cccc7c6c6ccccc6n7-c6ccccc6)ccc5c5ncccc54)cc3)n2)cc1 ; c1ccc(-c2cc(-c3ccc(-n4c5cc(-c6cccc7c6c6ccccc6n7-c6ccccc6)ccc5c5ncccc54)cc3)nc(-c3ccccc3)n2)cc1 ; c1ccc(-c2cc(-c3ccc(-n4c5cc(-c6cccc7c6c6ccccc6n7-c6ccccc6)ccc5c5ncccc54)cc3)cc(-c3ccccc3)n2)cc1]

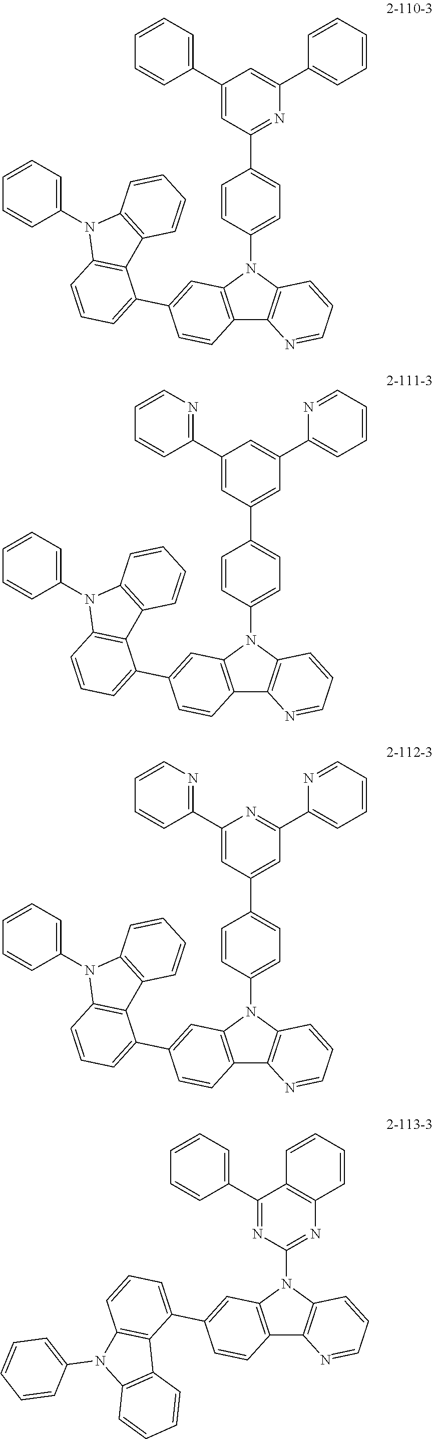 [SMILES: c1ccc(-c2cc(-c3ccccc3)nc(-c3ccc(-n4c5cc(-c6cccc7c6c6ccccc6n7-c6ccccc6)ccc5c5ncccc54)cc3)c2)cc1 ; c1ccc(-n2c3ccccc3c3c(-c4ccc5c6ncccc6n(-c6ccc(-c7cc(-c8ccccn8)cc(-c8ccccn8)c7)cc6)c5c4)cccc32)cc1 ; c1ccc(-n2c3ccccc3c3c(-c4ccc5c6ncccc6n(-c6ccc(-c7cc(-c8ccccn8)nc(-c8ccccn8)c7)cc6)c5c4)cccc32)cc1 ; c1ccc(-c2nc(-n3c4cc(-c5cccc6c5c5ccccc5n6-c5ccccc5)ccc4c4ncccc43)nc3ccccc23)cc1]

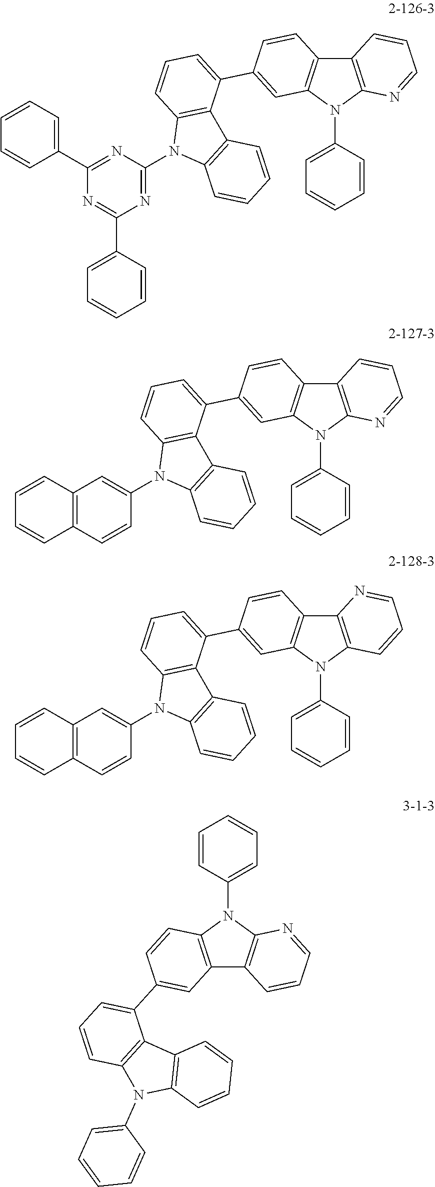 [SMILES: c1ccc(-c2nc(-c3ccccc3)nc(-n3c4ccccc4c4c(-c5ccc6c7cccnc7n(-c7ccccc7)c6c5)cccc43)n2)cc1 ; c1ccc(-n2c3cc(-c4cccc5c4c4ccccc4n5-c4ccc5ccccc5c4)ccc3c3cccnc32)cc1 ; c1ccc(-n2c3cc(-c4cccc5c4c4ccccc4n5-c4ccc5ccccc5c4)ccc3c3ncccc32)cc1 ; c1ccc(-n2c3ccccc3c3c(-c4ccc5c(c4)c4cccnc4n5-c4ccccc4)cccc32)cc1]

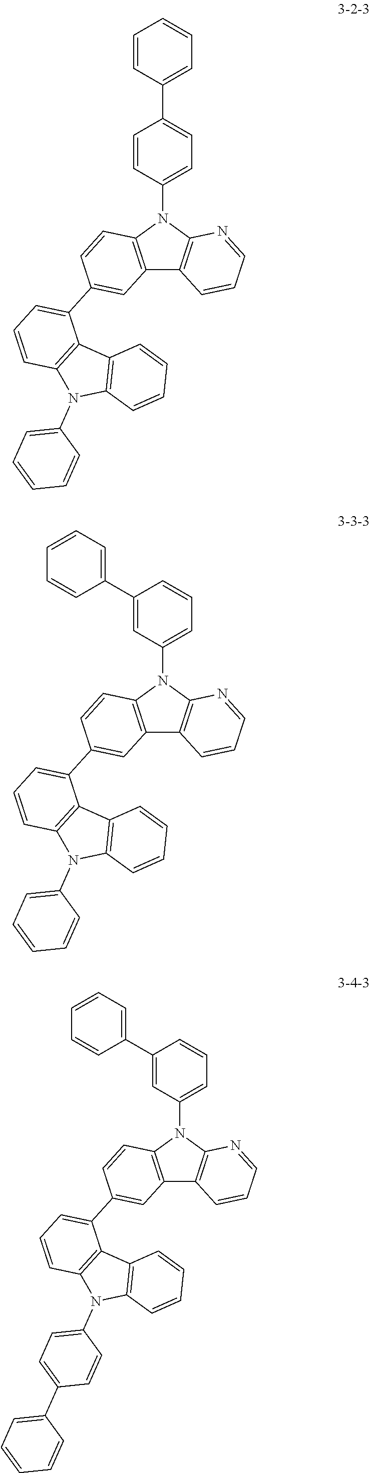

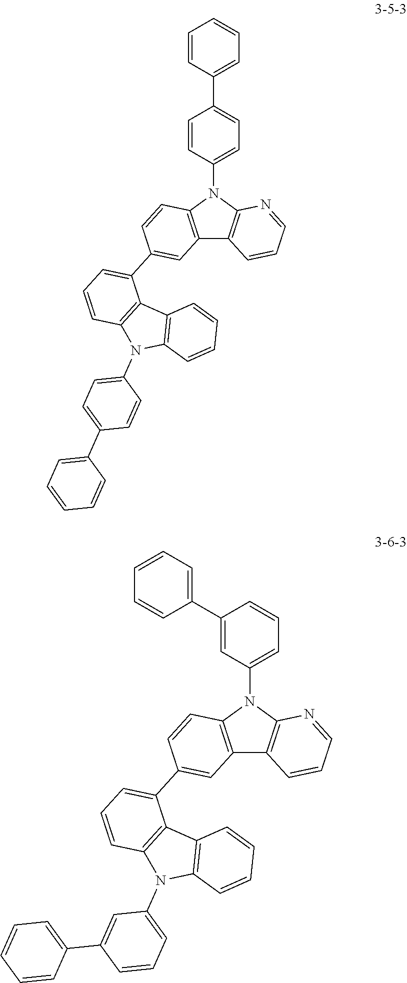 [SMILES: c1ccc(-c2ccc(-n3c4ccccc4c4c(-c5ccc6c(c5)c5cccnc5n6-c5ccc(-c6ccccc6)cc5)cccc43)cc2)cc1 ; c1ccc(-c2cccc(-n3c4ccccc4c4c(-c5ccc6c(c5)c5cccnc5n6-c5cccc(-c6ccccc6)c5)cccc43)c2)cc1]

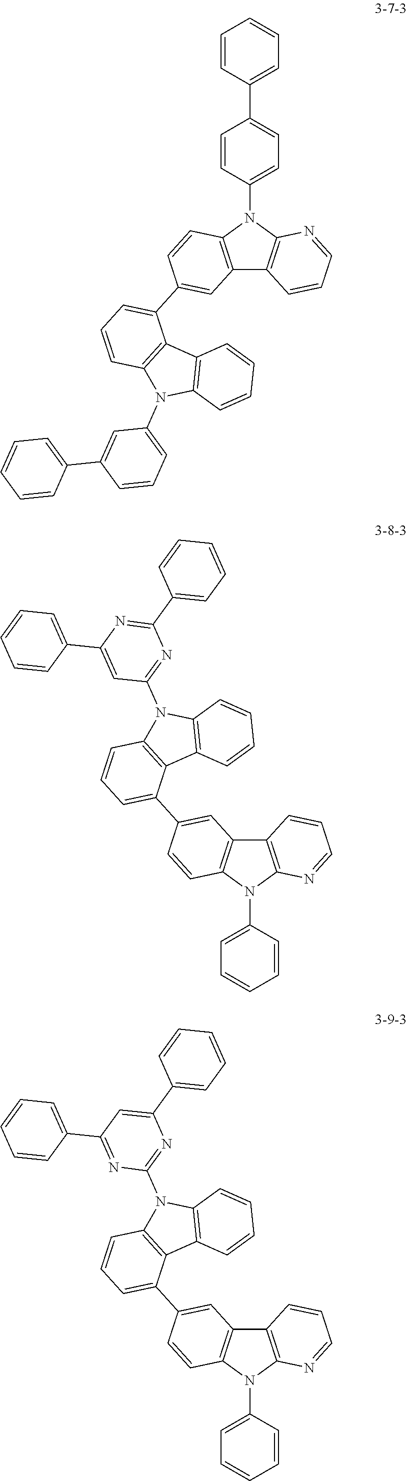 [SMILES: c1ccc(-c2ccc(-n3c4ccc(-c5cccc6c5c5ccccc5n6-c5cccc(-c6ccccc6)c5)cc4c4cccnc43)cc2)cc1 ; c1ccc(-c2cc(-n3c4ccccc4c4c(-c5ccc6c(c5)c5cccnc5n6-c5ccccc5)cccc43)nc(-c3ccccc3)n2)cc1 ; c1ccc(-c2cc(-c3ccccc3)nc(-n3c4ccccc4c4c(-c5ccc6c(c5)c5cccnc5n6-c5ccccc5)cccc43)n2)cc1]

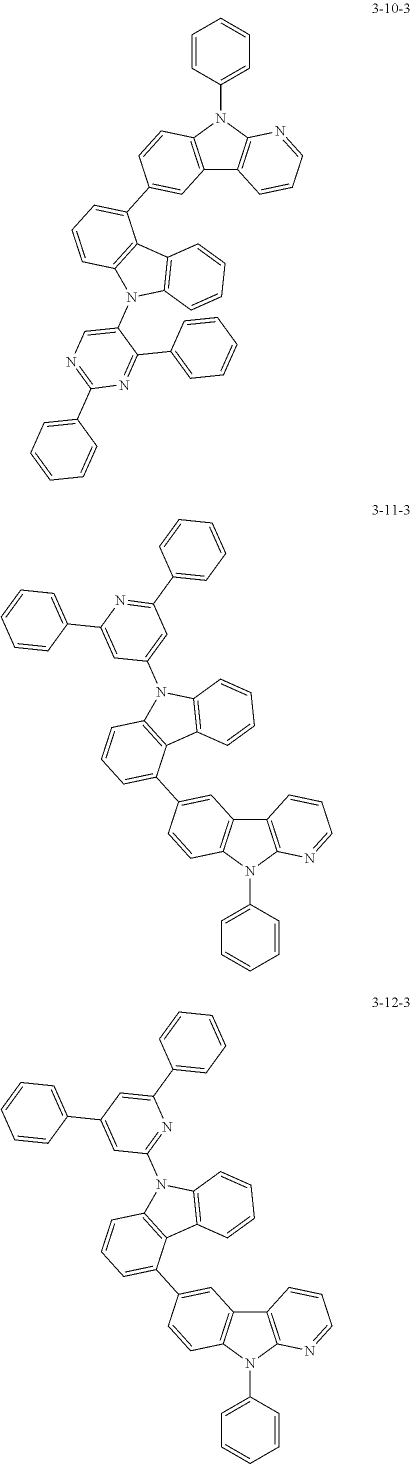 [SMILES: c1ccc(-c2ncc(-n3c4ccccc4c4c(-c5ccc6c(c5)c5cccnc5n6-c5ccccc5)cccc43)c(-c3ccccc3)n2)cc1 ; c1ccc(-c2cc(-n3c4ccccc4c4c(-c5ccc6c(c5)c5cccnc5n6-c5ccccc5)cccc43)cc(-c3ccccc3)n2)cc1 ; c1ccc(-c2cc(-c3ccccc3)nc(-n3c4ccccc4c4c(-c5ccc6c(c5)c5cccnc5n6-c5ccccc5)cccc43)c2)cc1]

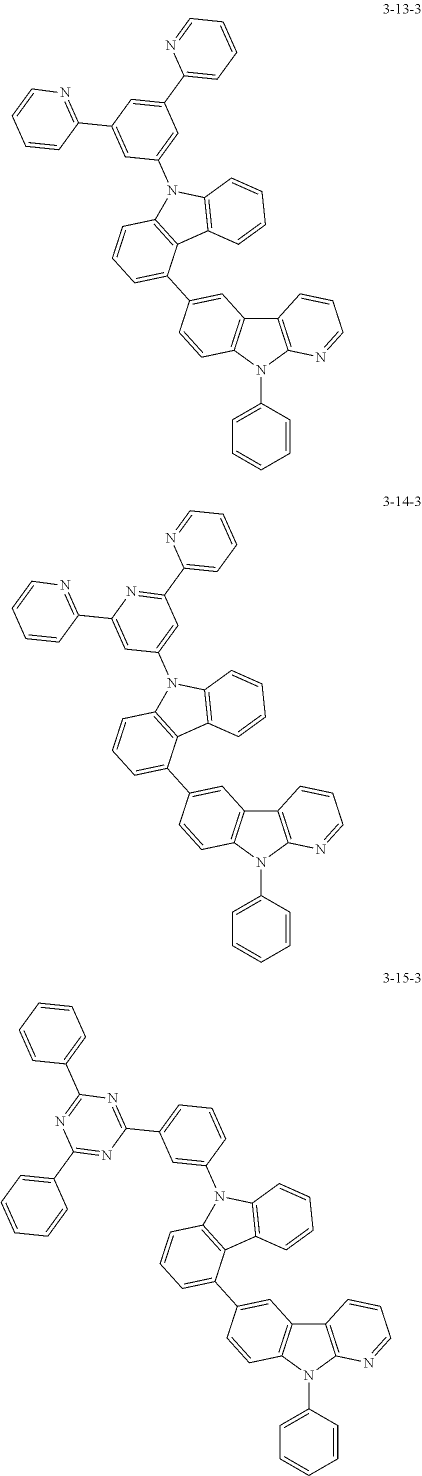 [SMILES: c1ccc(-n2c3ccc(-c4cccc5c4c4ccccc4n5-c4cc(-c5ccccn5)cc(-c5ccccn5)c4)cc3c3cccnc32)cc1 ; c1ccc(-n2c3ccc(-c4cccc5c4c4ccccc4n5-c4cc(-c5ccccn5)nc(-c5ccccn5)c4)cc3c3cccnc32)cc1 ; c1ccc(-c2nc(-c3ccccc3)nc(-c3cccc(-n4c5ccccc5c5c(-c6ccc7c(c6)c6cccnc6n7-c6ccccc6)cccc54)c3)n2)cc1]

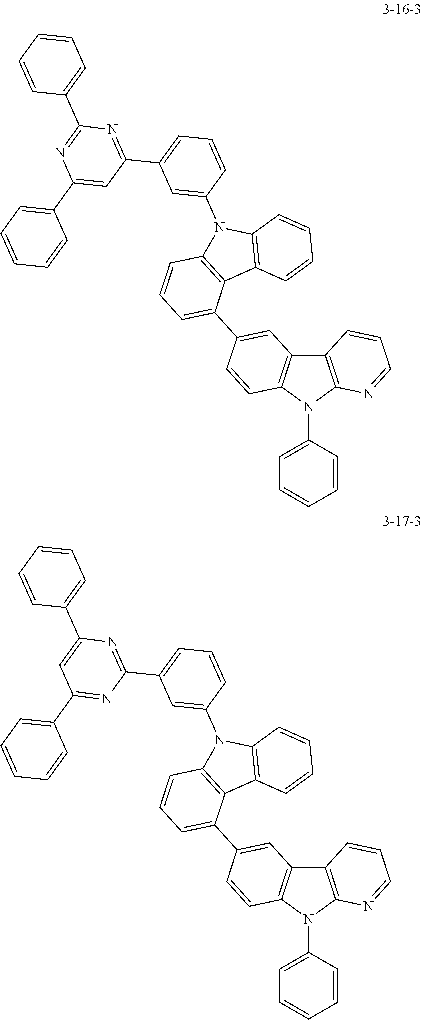 [SMILES: c1ccc(-c2cc(-c3cccc(-n4c5ccccc5c5c(-c6ccc7c(c6)c6cccnc6n7-c6ccccc6)cccc54)c3)nc(-c3ccccc3)n2)cc1 ; c1ccc(-c2cc(-c3ccccc3)nc(-c3cccc(-n4c5ccccc5c5c(-c6ccc7c(c6)c6cccnc6n7-c6ccccc6)cccc54)c3)n2)cc1]

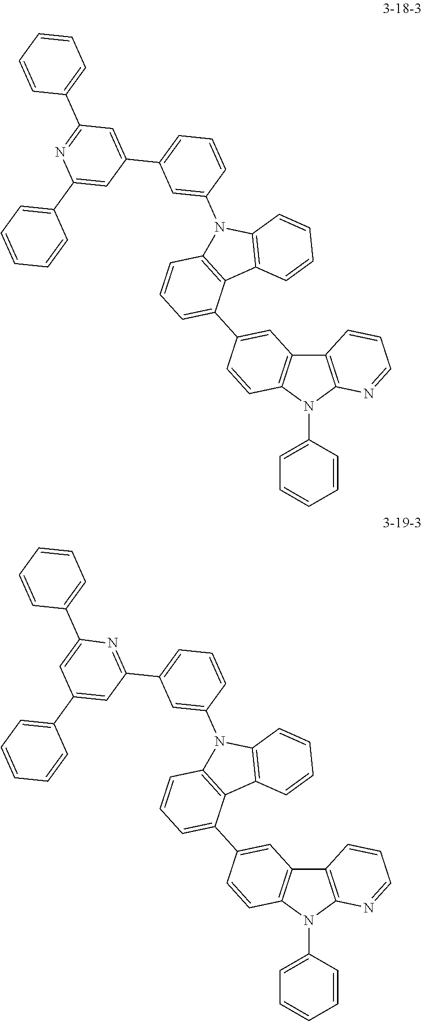 [SMILES: c1ccc(-c2cc(-c3cccc(-n4c5ccccc5c5c(-c6ccc7c(c6)c6cccnc6n7-c6ccccc6)cccc54)c3)cc(-c3ccccc3)n2)cc1 ; c1ccc(-c2cc(-c3ccccc3)nc(-c3cccc(-n4c5ccccc5c5c(-c6ccc7c(c6)c6cccnc6n7-c6ccccc6)cccc54)c3)c2)cc1]

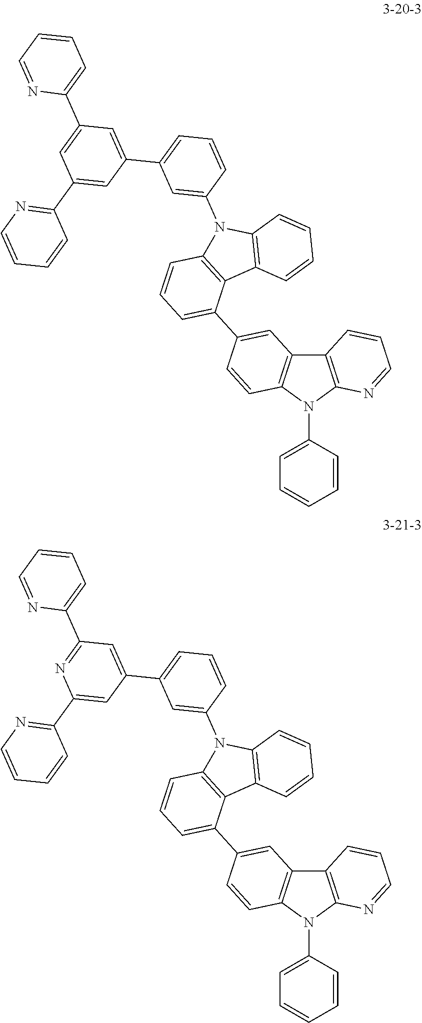 [SMILES: c1ccc(-n2c3ccc(-c4cccc5c4c4ccccc4n5-c4cccc(-c5cc(-c6ccccn6)cc(-c6ccccn6)c5)c4)cc3c3cccnc32)cc1 ; c1ccc(-n2c3ccc(-c4cccc5c4c4ccccc4n5-c4cccc(-c5cc(-c6ccccn6)nc(-c6ccccn6)c5)c4)cc3c3cccnc32)cc1]

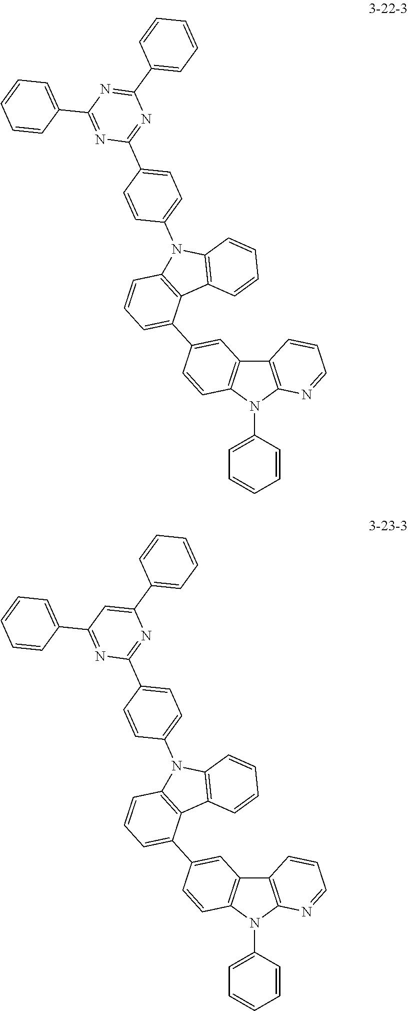 [SMILES: c1ccc(-c2nc(-c3ccccc3)nc(-c3ccc(-n4c5ccccc5c5c(-c6ccc7c(c6)c6cccnc6n7-c6ccccc6)cccc54)cc3)n2)cc1 ; c1ccc(-c2cc(-c3ccccc3)nc(-c3ccc(-n4c5ccccc5c5c(-c6ccc7c(c6)c6cccnc6n7-c6ccccc6)cccc54)cc3)n2)cc1]

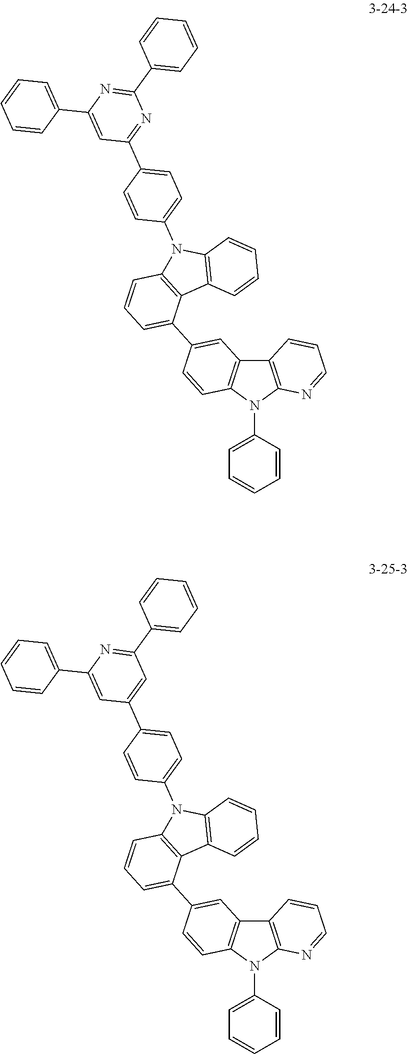 [SMILES: c1ccc(-c2cc(-c3ccc(-n4c5ccccc5c5c(-c6ccc7c(c6)c6cccnc6n7-c6ccccc6)cccc54)cc3)nc(-c3ccccc3)n2)cc1 ; c1ccc(-c2cc(-c3ccc(-n4c5ccccc5c5c(-c6ccc7c(c6)c6cccnc6n7-c6ccccc6)cccc54)cc3)cc(-c3ccccc3)n2)cc1]

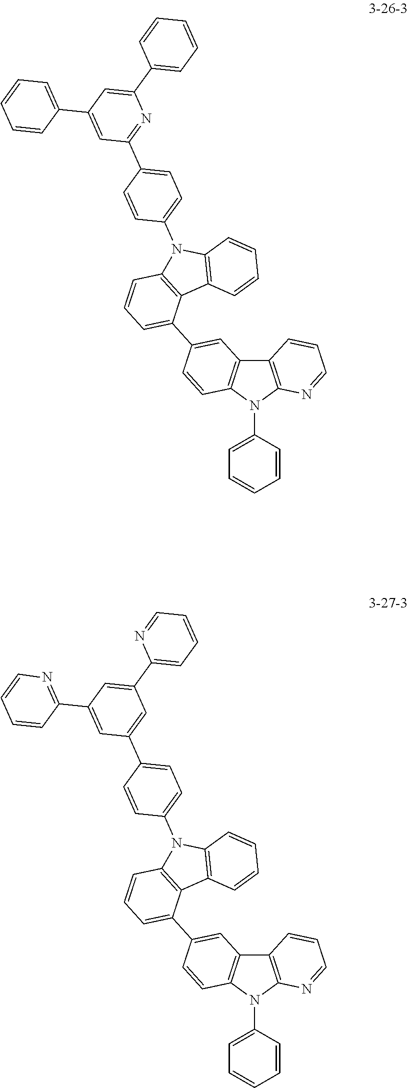 [SMILES: c1ccc(-c2cc(-c3ccccc3)nc(-c3ccc(-n4c5ccccc5c5c(-c6ccc7c(c6)c6cccnc6n7-c6ccccc6)cccc54)cc3)c2)cc1 ; c1ccc(-n2c3ccc(-c4cccc5c4c4ccccc4n5-c4ccc(-c5cc(-c6ccccn6)cc(-c6ccccn6)c5)cc4)cc3c3cccnc32)cc1]

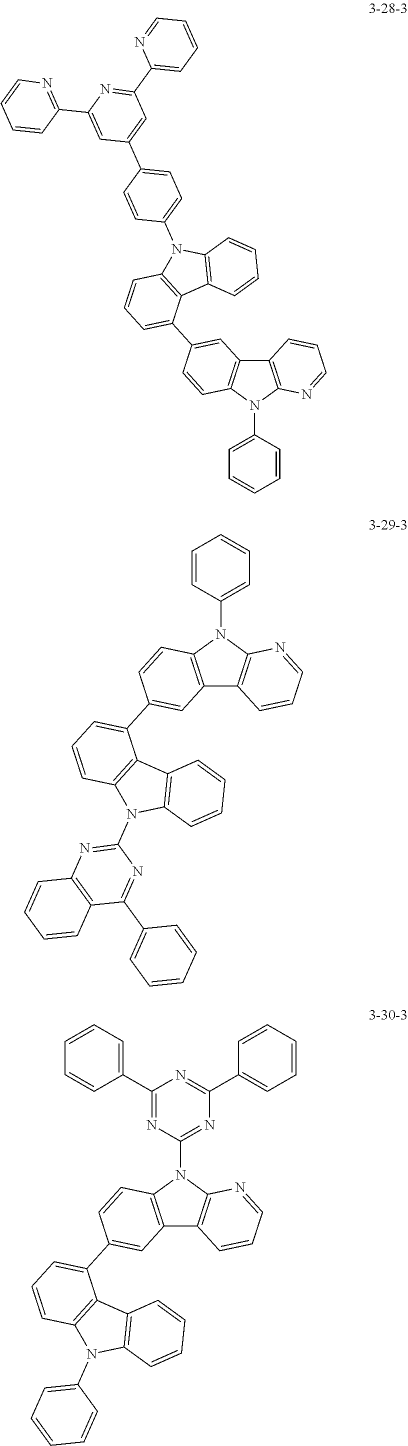 [SMILES: c1ccc(-n2c3ccc(-c4cccc5c4c4ccccc4n5-c4ccc(-c5cc(-c6ccccn6)nc(-c6ccccn6)c5)cc4)cc3c3cccnc32)cc1 ; c1ccc(-c2nc(-n3c4ccccc4c4c(-c5ccc6c(c5)c5cccnc5n6-c5ccccc5)cccc43)nc3ccccc23)cc1 ; c1ccc(-c2nc(-c3ccccc3)nc(-n3c4ccc(-c5cccc6c5c5ccccc5n6-c5ccccc5)cc4c4cccnc43)n2)cc1]

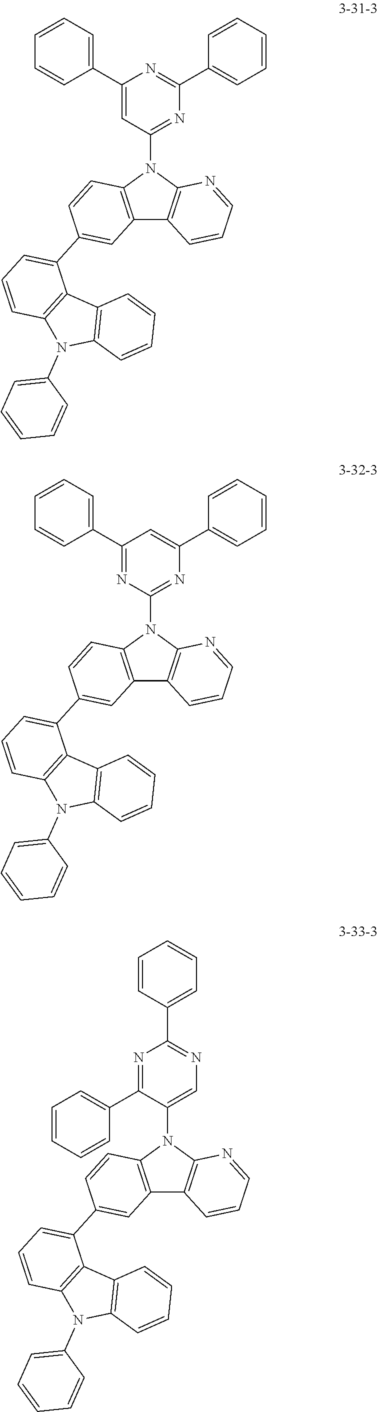

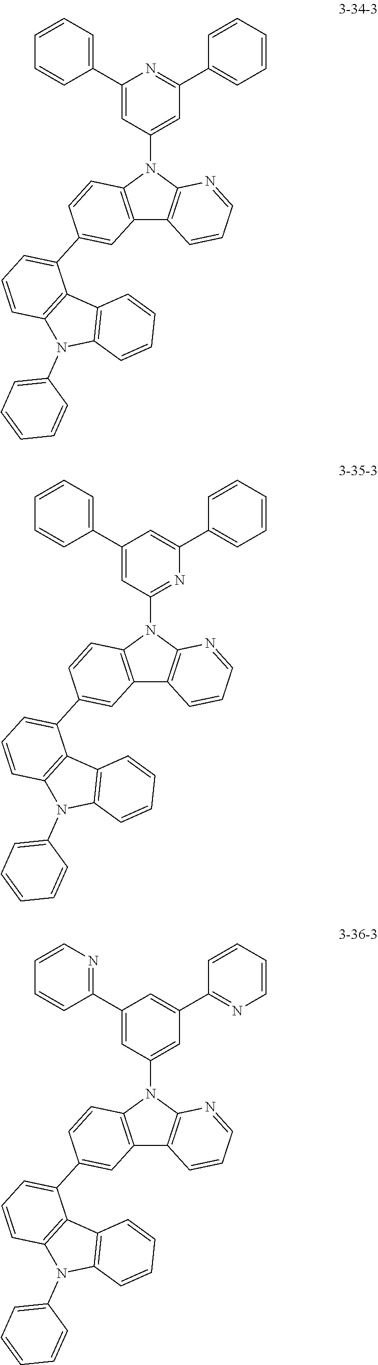

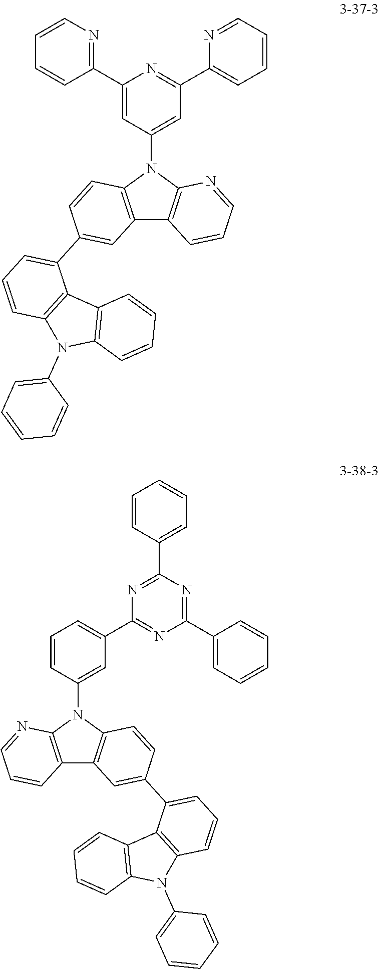 [SMILES: c1ccc(-n2c3ccccc3c3c(-c4ccc5c(c4)c4cccnc4n5-c4cc(-c5ccccn5)nc(-c5ccccn5)c4)cccc32)cc1 ; c1ccc(-c2nc(-c3ccccc3)nc(-c3cccc(-n4c5ccc(-c6cccc7c6c6ccccc6n7-c6ccccc6)cc5c5cccnc54)c3)n2)cc1]

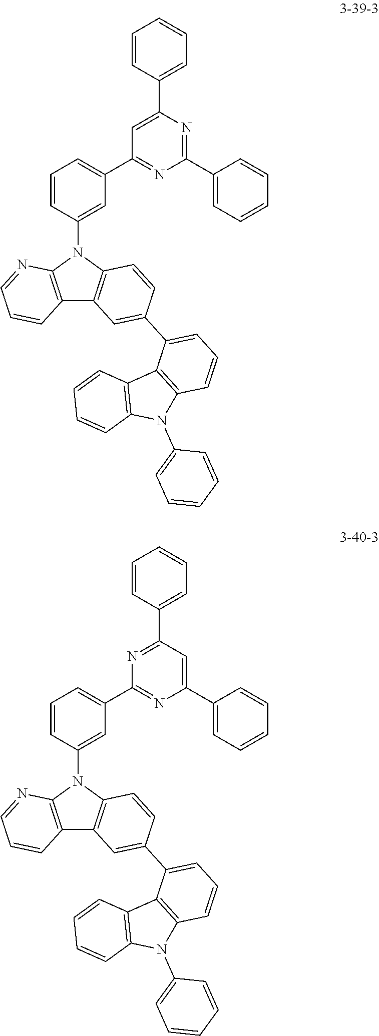 [SMILES: c1ccc(-c2cc(-c3cccc(-n4c5ccc(-c6cccc7c6c6ccccc6n7-c6ccccc6)cc5c5cccnc54)c3)nc(-c3ccccc3)n2)cc1 ; c1ccc(-c2cc(-c3ccccc3)nc(-c3cccc(-n4c5ccc(-c6cccc7c6c6ccccc6n7-c6ccccc6)cc5c5cccnc54)c3)n2)cc1]

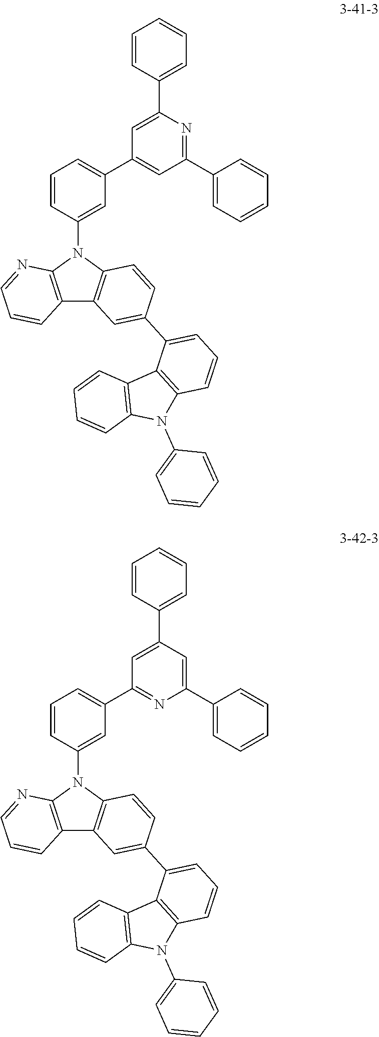 [SMILES: c1ccc(-c2cc(-c3cccc(-n4c5ccc(-c6cccc7c6c6ccccc6n7-c6ccccc6)cc5c5cccnc54)c3)cc(-c3ccccc3)n2)cc1 ; c1ccc(-c2cc(-c3ccccc3)nc(-c3cccc(-n4c5ccc(-c6cccc7c6c6ccccc6n7-c6ccccc6)cc5c5cccnc54)c3)c2)cc1]

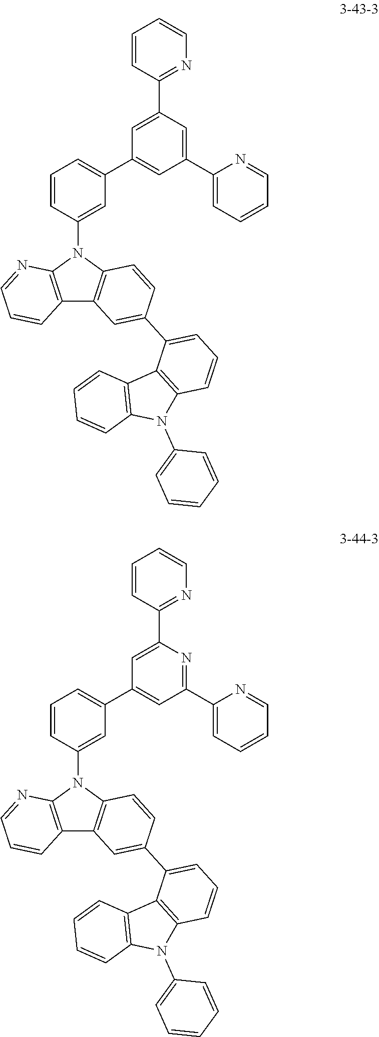 [SMILES: c1ccc(-n2c3ccccc3c3c(-c4ccc5c(c4)c4cccnc4n5-c4cccc(-c5cc(-c6ccccn6)cc(-c6ccccn6)c5)c4)cccc32)cc1 ; c1ccc(-n2c3ccccc3c3c(-c4ccc5c(c4)c4cccnc4n5-c4cccc(-c5cc(-c6ccccn6)nc(-c6ccccn6)c5)c4)cccc32)cc1]

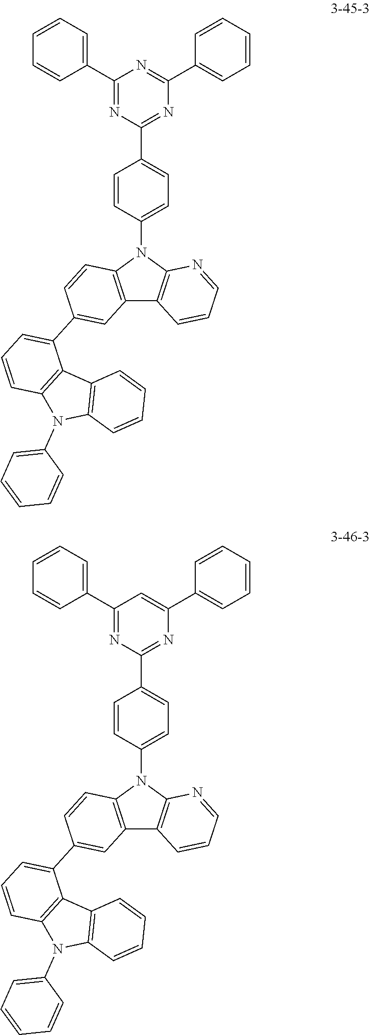 [SMILES: c1ccc(-c2nc(-c3ccccc3)nc(-c3ccc(-n4c5ccc(-c6cccc7c6c6ccccc6n7-c6ccccc6)cc5c5cccnc54)cc3)n2)cc1 ; c1ccc(-c2cc(-c3ccccc3)nc(-c3ccc(-n4c5ccc(-c6cccc7c6c6ccccc6n7-c6ccccc6)cc5c5cccnc54)cc3)n2)cc1]

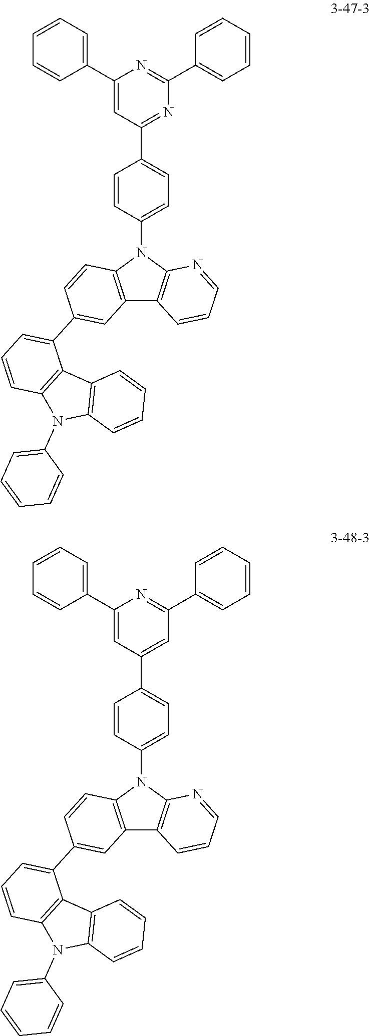 [SMILES: c1ccc(-c2cc(-c3ccc(-n4c5ccc(-c6cccc7c6c6ccccc6n7-c6ccccc6)cc5c5cccnc54)cc3)nc(-c3ccccc3)n2)cc1 ; c1ccc(-c2cc(-c3ccc(-n4c5ccc(-c6cccc7c6c6ccccc6n7-c6ccccc6)cc5c5cccnc54)cc3)cc(-c3ccccc3)n2)cc1]

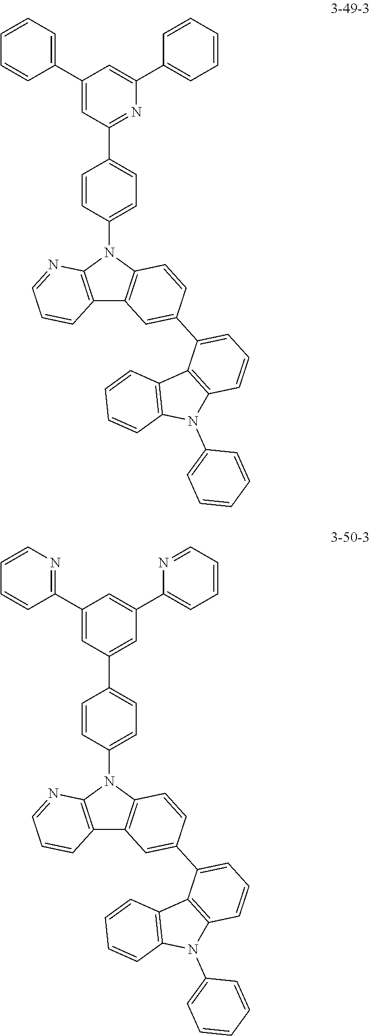 [SMILES: c1ccc(-c2cc(-c3ccccc3)nc(-c3ccc(-n4c5ccc(-c6cccc7c6c6ccccc6n7-c6ccccc6)cc5c5cccnc54)cc3)c2)cc1 ; c1ccc(-n2c3ccccc3c3c(-c4ccc5c(c4)c4cccnc4n5-c4ccc(-c5cc(-c6ccccn6)cc(-c6ccccn6)c5)cc4)cccc32)cc1]

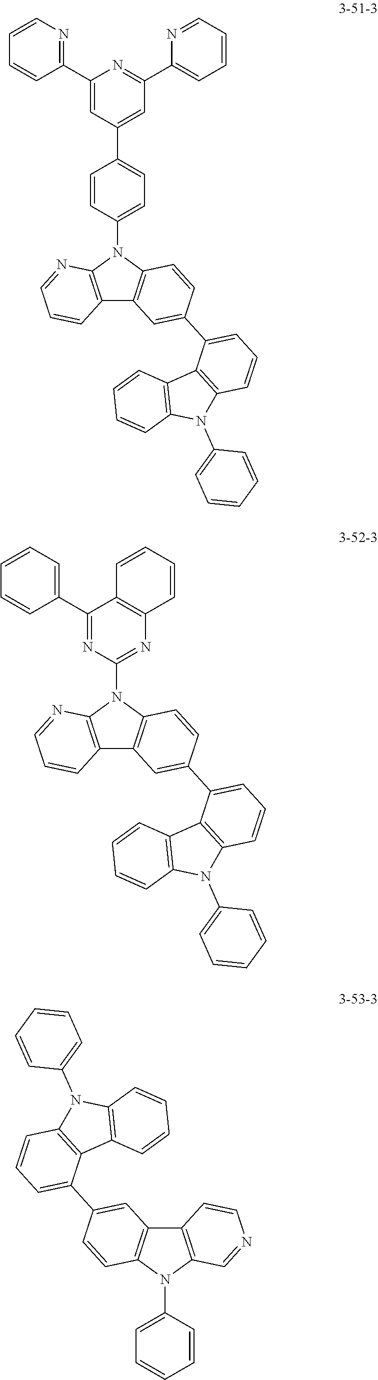 [SMILES: c1ccc(-n2c3ccccc3c3c(-c4ccc5c(c4)c4cccnc4n5-c4ccc(-c5cc(-c6ccccn6)nc(-c6ccccn6)c5)cc4)cccc32)cc1 ; c1ccc(-c2nc(-n3c4ccc(-c5cccc6c5c5ccccc5n6-c5ccccc5)cc4c4cccnc43)nc3ccccc23)cc1 ; c1ccc(-n2c3ccc(-c4cccc5c4c4ccccc4n5-c4ccccc4)cc3c3ccncc32)cc1]

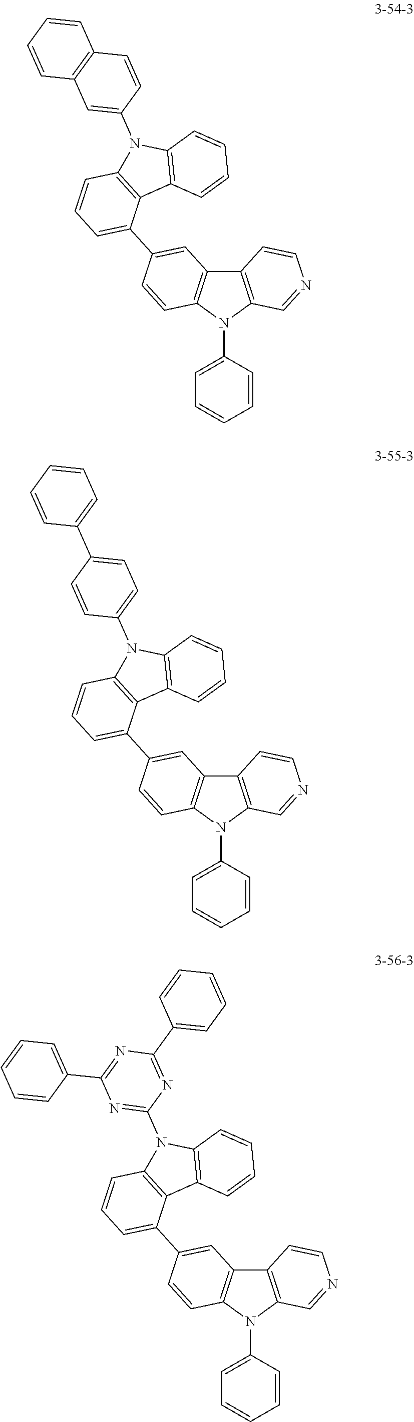 [SMILES: c1ccc(-n2c3ccc(-c4cccc5c4c4ccccc4n5-c4ccc5ccccc5c4)cc3c3ccncc32)cc1 ; c1ccc(-c2ccc(-n3c4ccccc4c4c(-c5ccc6c(c5)c5ccncc5n6-c5ccccc5)cccc43)cc2)cc1 ; c1ccc(-c2nc(-c3ccccc3)nc(-n3c4ccccc4c4c(-c5ccc6c(c5)c5ccncc5n6-c5ccccc5)cccc43)n2)cc1]

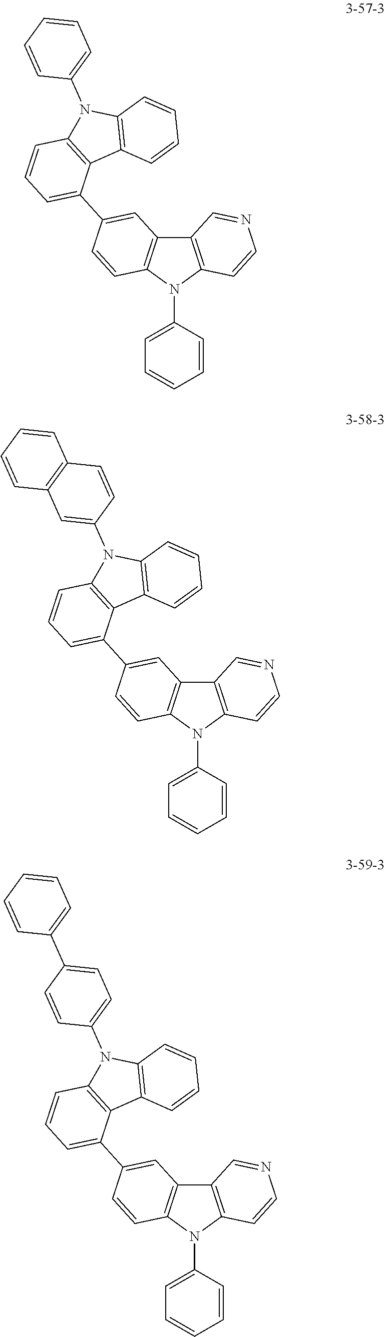 [SMILES: c1ccc(-n2c3ccncc3c3cc(-c4cccc5c4c4ccccc4n5-c4ccccc4)ccc32)cc1 ; c1ccc(-n2c3ccncc3c3cc(-c4cccc5c4c4ccccc4n5-c4ccc5ccccc5c4)ccc32)cc1 ; c1ccc(-c2ccc(-n3c4ccccc4c4c(-c5ccc6c(c5)c5cnccc5n6-c5ccccc5)cccc43)cc2)cc1]

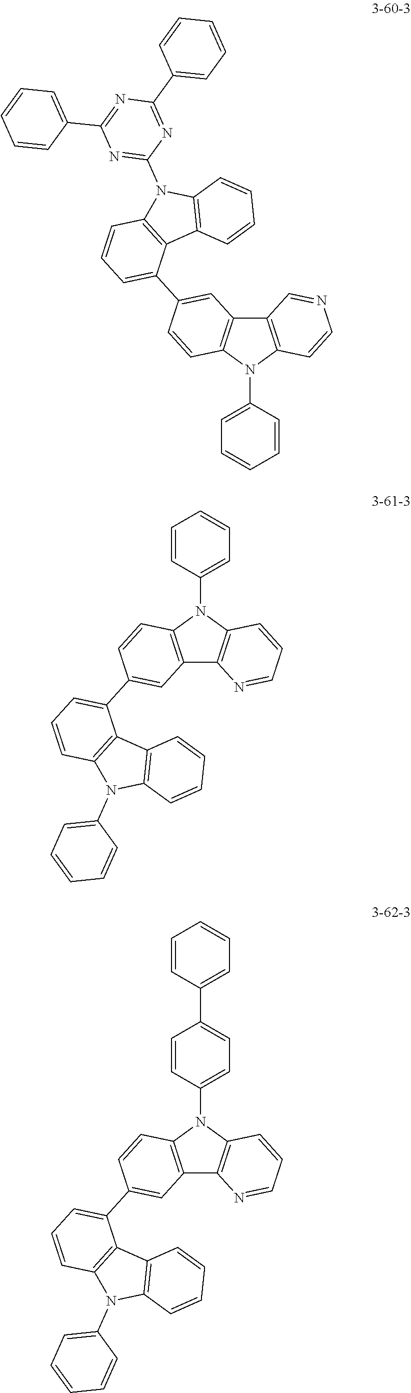 [SMILES: c1ccc(-c2nc(-c3ccccc3)nc(-n3c4ccccc4c4c(-c5ccc6c(c5)c5cnccc5n6-c5ccccc5)cccc43)n2)cc1 ; c1ccc(-n2c3ccc(-c4cccc5c4c4ccccc4n5-c4ccccc4)cc3c3ncccc32)cc1 ; c1ccc(-c2ccc(-n3c4ccc(-c5cccc6c5c5ccccc5n6-c5ccccc5)cc4c4ncccc43)cc2)cc1]

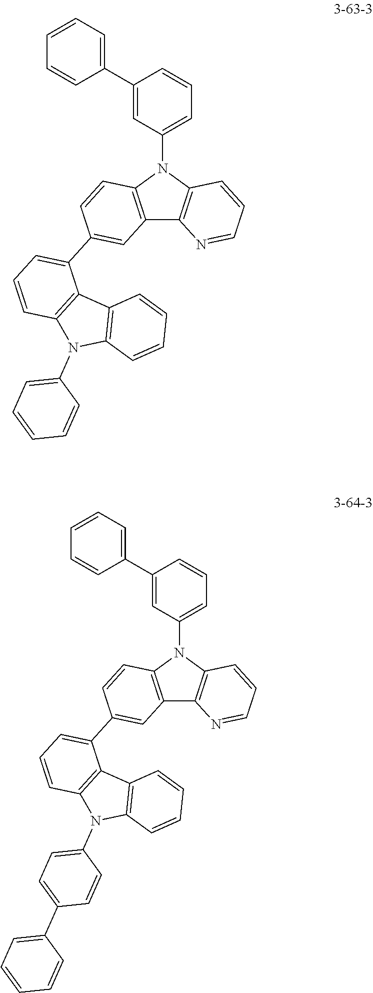 [SMILES: c1ccc(-c2cccc(-n3c4ccc(-c5cccc6c5c5ccccc5n6-c5ccccc5)cc4c4ncccc43)c2)cc1 ; c1ccc(-c2ccc(-n3c4ccccc4c4c(-c5ccc6c(c5)c5ncccc5n6-c5cccc(-c6ccccc6)c5)cccc43)cc2)cc1]

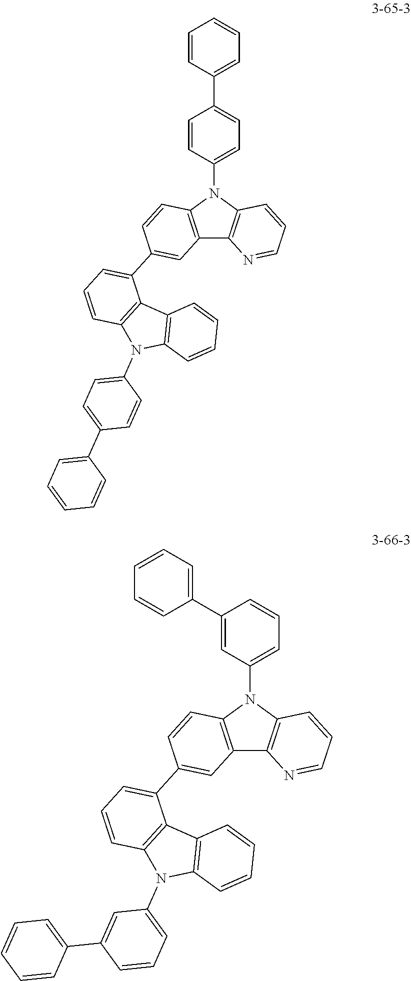 [SMILES: c1ccc(-c2ccc(-n3c4ccc(-c5cccc6c5c5ccccc5n6-c5ccc(-c6ccccc6)cc5)cc4c4ncccc43)cc2)cc1 ; c1ccc(-c2cccc(-n3c4ccc(-c5cccc6c5c5ccccc5n6-c5cccc(-c6ccccc6)c5)cc4c4ncccc43)c2)cc1]

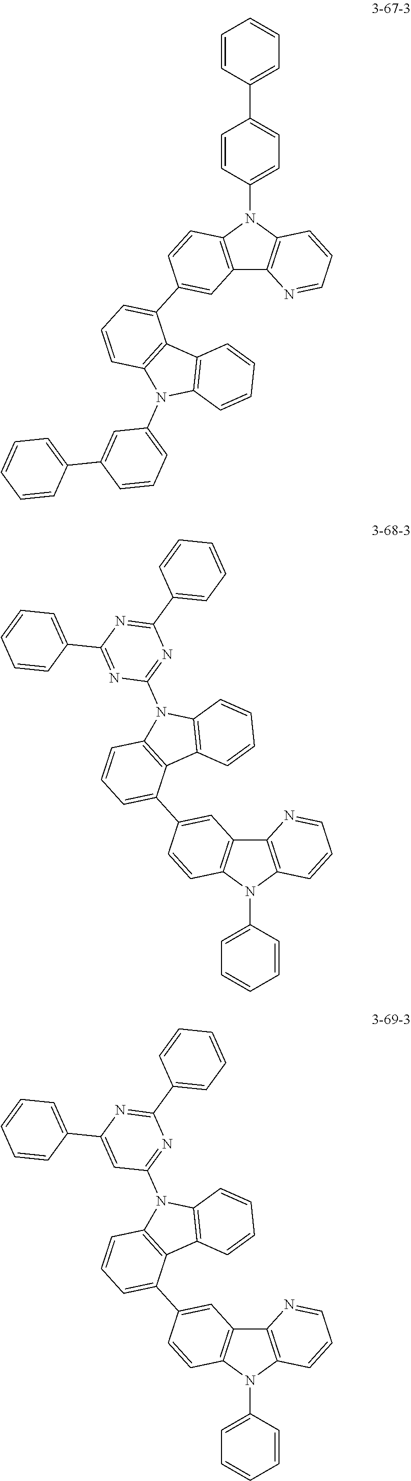 [SMILES: c1ccc(-c2ccc(-n3c4ccc(-c5cccc6c5c5ccccc5n6-c5cccc(-c6ccccc6)c5)cc4c4ncccc43)cc2)cc1 ; c1ccc(-c2nc(-c3ccccc3)nc(-n3c4ccccc4c4c(-c5ccc6c(c5)c5ncccc5n6-c5ccccc5)cccc43)n2)cc1 ; c1ccc(-c2cc(-n3c4ccccc4c4c(-c5ccc6c(c5)c5ncccc5n6-c5ccccc5)cccc43)nc(-c3ccccc3)n2)cc1]

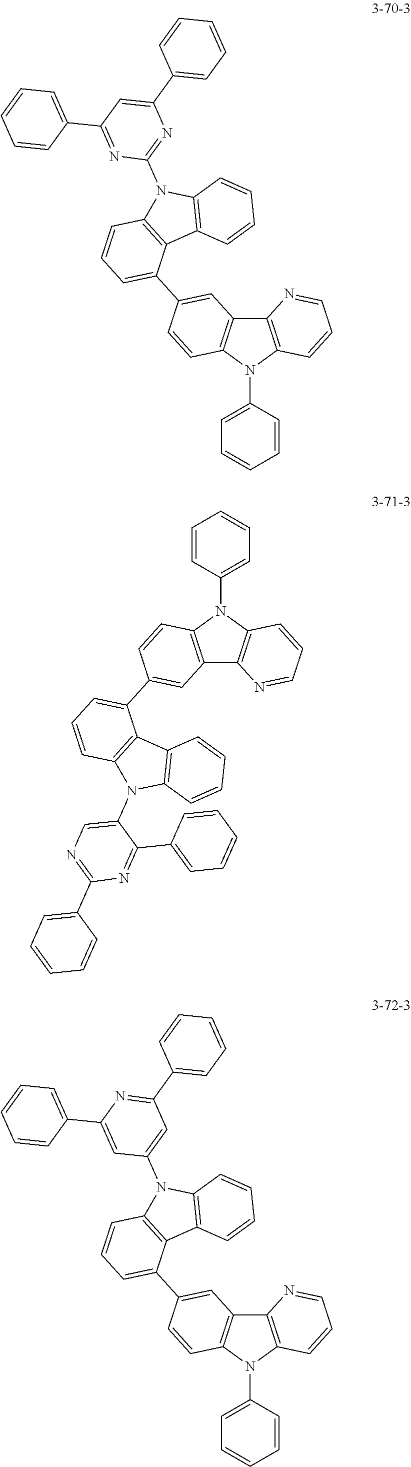 [SMILES: c1ccc(-c2cc(-c3ccccc3)nc(-n3c4ccccc4c4c(-c5ccc6c(c5)c5ncccc5n6-c5ccccc5)cccc43)n2)cc1 ; c1ccc(-c2ncc(-n3c4ccccc4c4c(-c5ccc6c(c5)c5ncccc5n6-c5ccccc5)cccc43)c(-c3ccccc3)n2)cc1 ; c1ccc(-c2cc(-n3c4ccccc4c4c(-c5ccc6c(c5)c5ncccc5n6-c5ccccc5)cccc43)cc(-c3ccccc3)n2)cc1]

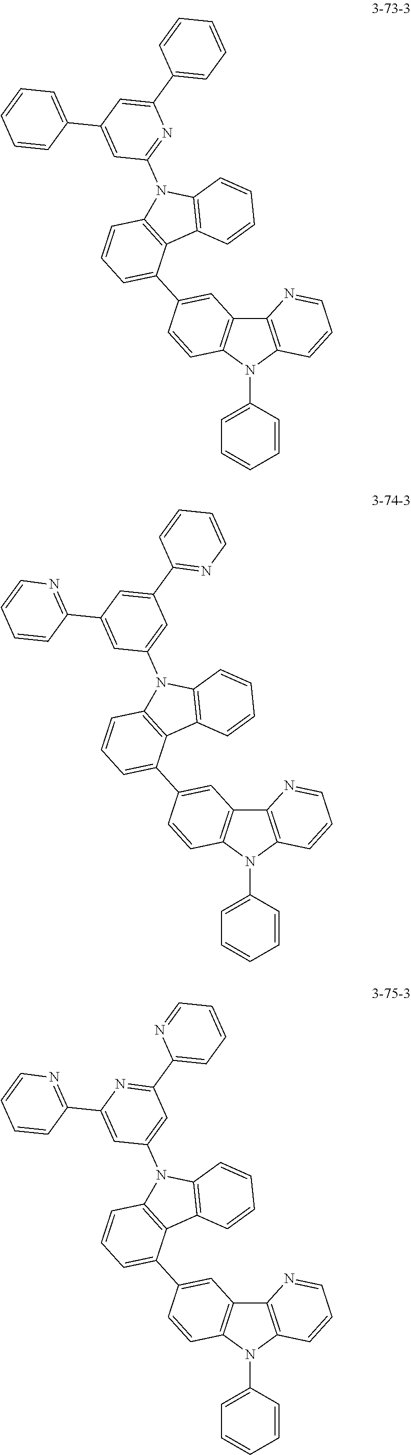 [SMILES: c1ccc(-c2cc(-c3ccccc3)nc(-n3c4ccccc4c4c(-c5ccc6c(c5)c5ncccc5n6-c5ccccc5)cccc43)c2)cc1 ; c1ccc(-n2c3ccc(-c4cccc5c4c4ccccc4n5-c4cc(-c5ccccn5)cc(-c5ccccn5)c4)cc3c3ncccc32)cc1 ; c1ccc(-n2c3ccc(-c4cccc5c4c4ccccc4n5-c4cc(-c5ccccn5)nc(-c5ccccn5)c4)cc3c3ncccc32)cc1]

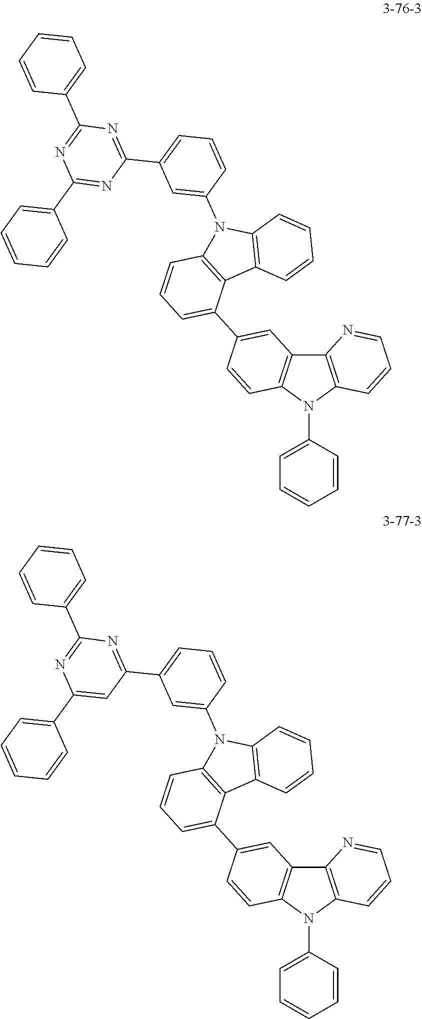 [SMILES: c1ccc(-c2nc(-c3ccccc3)nc(-c3cccc(-n4c5ccccc5c5c(-c6ccc7c(c6)c6ncccc6n7-c6ccccc6)cccc54)c3)n2)cc1 ; c1ccc(-c2cc(-c3cccc(-n4c5ccccc5c5c(-c6ccc7c(c6)c6ncccc6n7-c6ccccc6)cccc54)c3)nc(-c3ccccc3)n2)cc1]

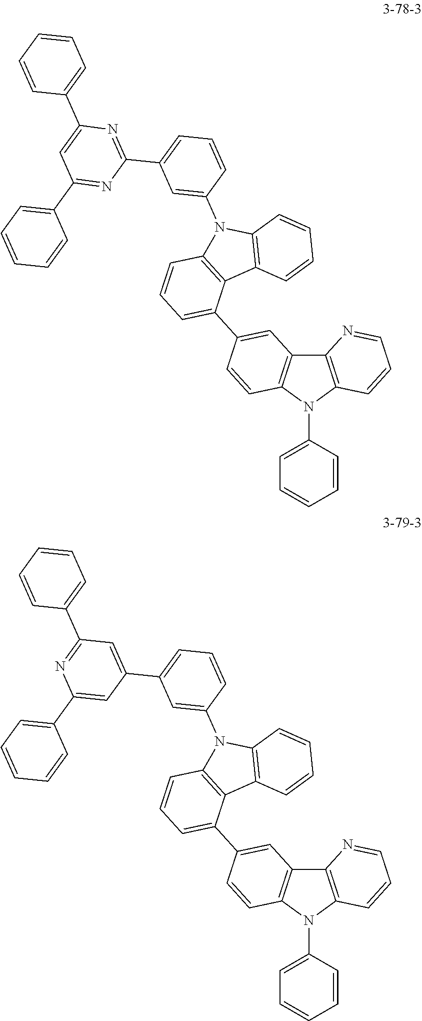 [SMILES: c1ccc(-c2cc(-c3ccccc3)nc(-c3cccc(-n4c5ccccc5c5c(-c6ccc7c(c6)c6ncccc6n7-c6ccccc6)cccc54)c3)n2)cc1 ; c1ccc(-c2cc(-c3cccc(-n4c5ccccc5c5c(-c6ccc7c(c6)c6ncccc6n7-c6ccccc6)cccc54)c3)cc(-c3ccccc3)n2)cc1]

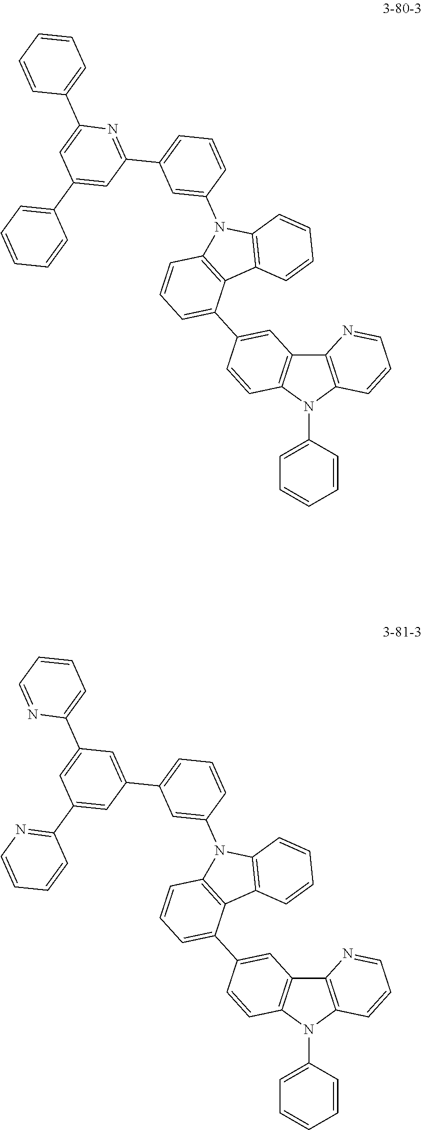 [SMILES: c1ccc(-c2cc(-c3ccccc3)nc(-c3cccc(-n4c5ccccc5c5c(-c6ccc7c(c6)c6ncccc6n7-c6ccccc6)cccc54)c3)c2)cc1 ; c1ccc(-n2c3ccc(-c4cccc5c4c4ccccc4n5-c4cccc(-c5cc(-c6ccccn6)cc(-c6ccccn6)c5)c4)cc3c3ncccc32)cc1]

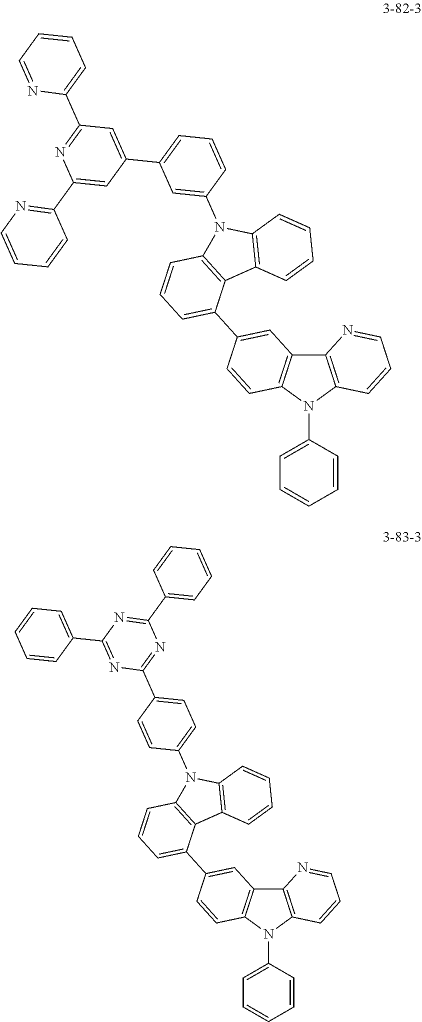 [SMILES: c1ccc(-n2c3ccc(-c4cccc5c4c4ccccc4n5-c4cccc(-c5cc(-c6ccccn6)nc(-c6ccccn6)c5)c4)cc3c3ncccc32)cc1 ; c1ccc(-c2nc(-c3ccccc3)nc(-c3ccc(-n4c5ccccc5c5c(-c6ccc7c(c6)c6ncccc6n7-c6ccccc6)cccc54)cc3)n2)cc1]

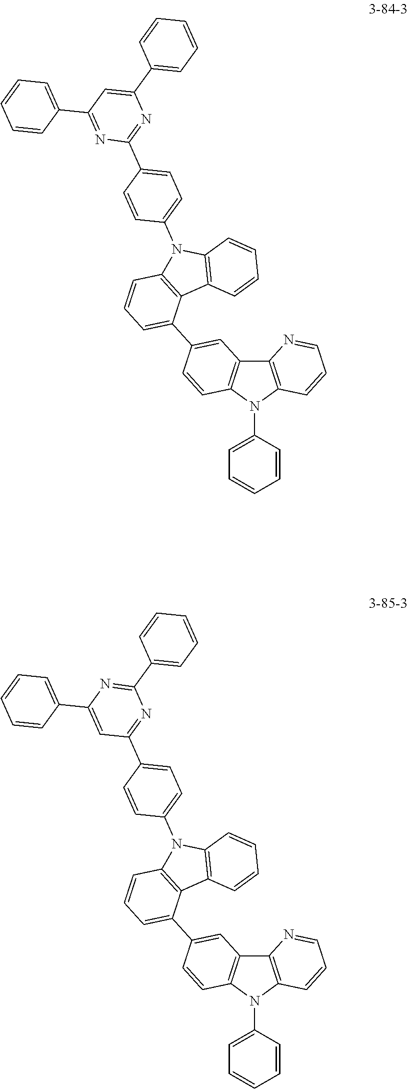 [SMILES: c1ccc(-c2cc(-c3ccccc3)nc(-c3ccc(-n4c5ccccc5c5c(-c6ccc7c(c6)c6ncccc6n7-c6ccccc6)cccc54)cc3)n2)cc1 ; c1ccc(-c2cc(-c3ccc(-n4c5ccccc5c5c(-c6ccc7c(c6)c6ncccc6n7-c6ccccc6)cccc54)cc3)nc(-c3ccccc3)n2)cc1]

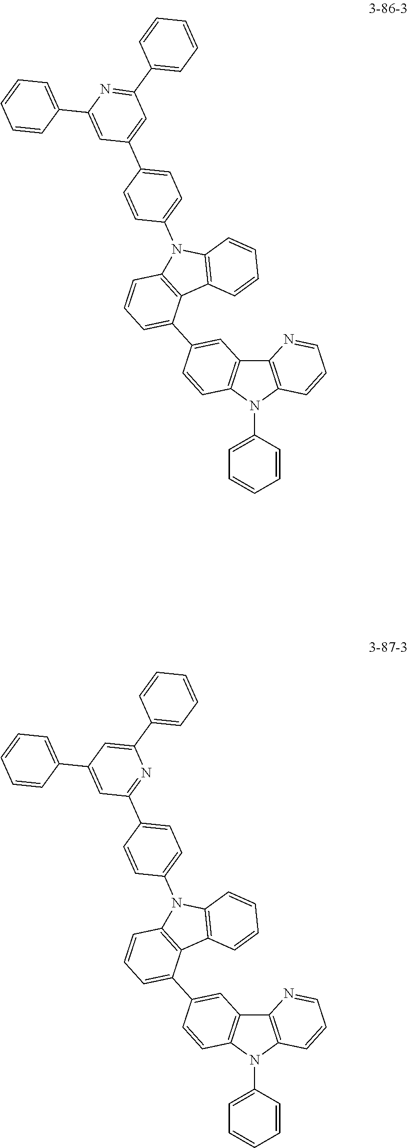 [SMILES: c1ccc(-c2cc(-c3ccc(-n4c5ccccc5c5c(-c6ccc7c(c6)c6ncccc6n7-c6ccccc6)cccc54)cc3)cc(-c3ccccc3)n2)cc1 ; c1ccc(-c2cc(-c3ccccc3)nc(-c3ccc(-n4c5ccccc5c5c(-c6ccc7c(c6)c6ncccc6n7-c6ccccc6)cccc54)cc3)c2)cc1]

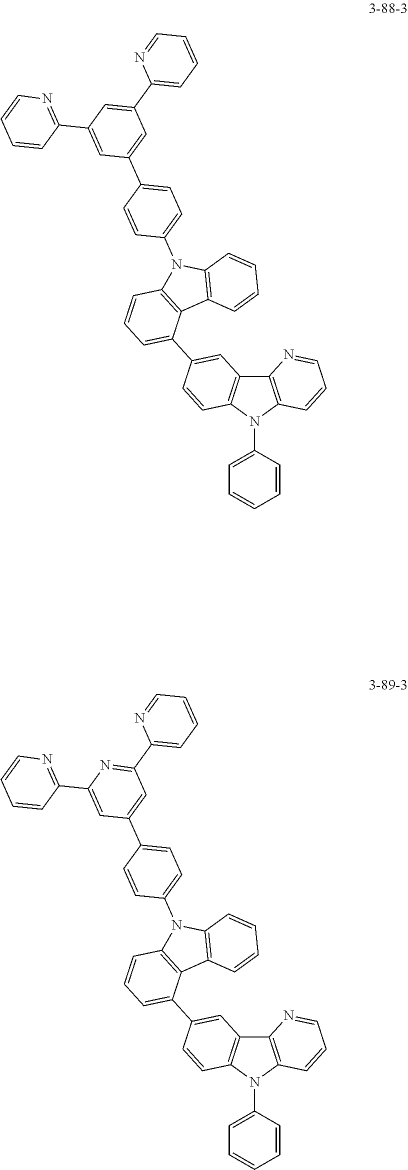 [SMILES: c1ccc(-n2c3ccc(-c4cccc5c4c4ccccc4n5-c4ccc(-c5cc(-c6ccccn6)cc(-c6ccccn6)c5)cc4)cc3c3ncccc32)cc1 ; c1ccc(-n2c3ccc(-c4cccc5c4c4ccccc4n5-c4ccc(-c5cc(-c6ccccn6)nc(-c6ccccn6)c5)cc4)cc3c3ncccc32)cc1]

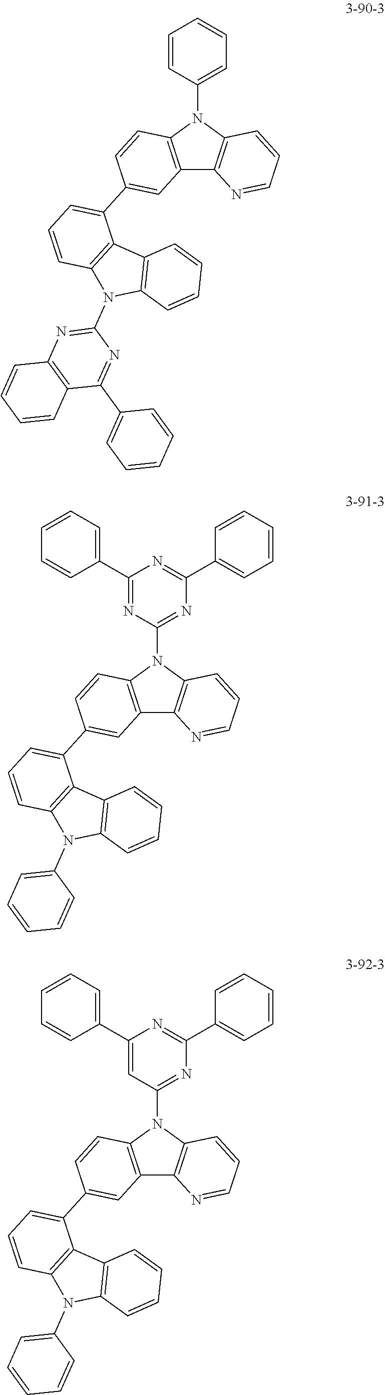 [SMILES: c1ccc(-c2nc(-n3c4ccccc4c4c(-c5ccc6c(c5)c5ncccc5n6-c5ccccc5)cccc43)nc3ccccc23)cc1 ; c1ccc(-c2nc(-c3ccccc3)nc(-n3c4ccc(-c5cccc6c5c5ccccc5n6-c5ccccc5)cc4c4ncccc43)n2)cc1 ; c1ccc(-c2cc(-n3c4ccc(-c5cccc6c5c5ccccc5n6-c5ccccc5)cc4c4ncccc43)nc(-c3ccccc3)n2)cc1]

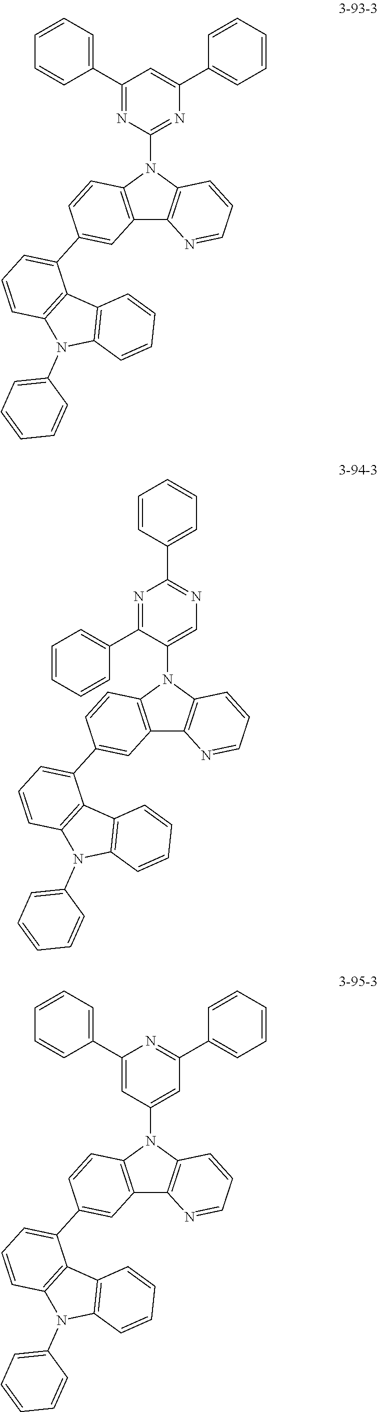

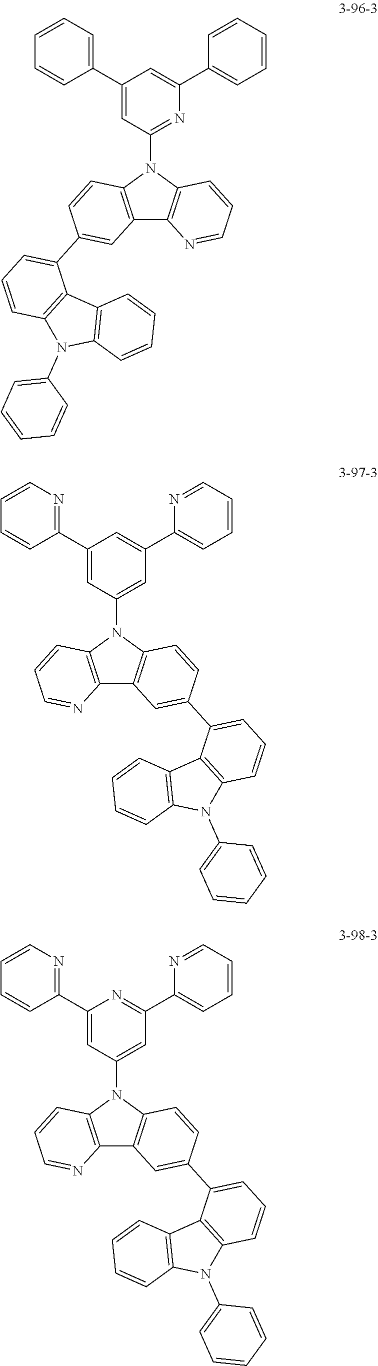

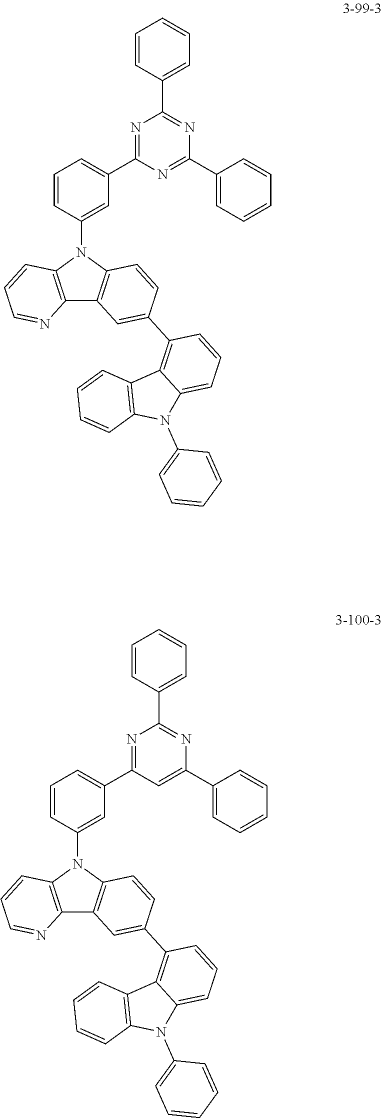 [SMILES: c1ccc(-c2nc(-c3ccccc3)nc(-c3cccc(-n4c5ccc(-c6cccc7c6c6ccccc6n7-c6ccccc6)cc5c5ncccc54)c3)n2)cc1 ; c1ccc(-c2cc(-c3cccc(-n4c5ccc(-c6cccc7c6c6ccccc6n7-c6ccccc6)cc5c5ncccc54)c3)nc(-c3ccccc3)n2)cc1]

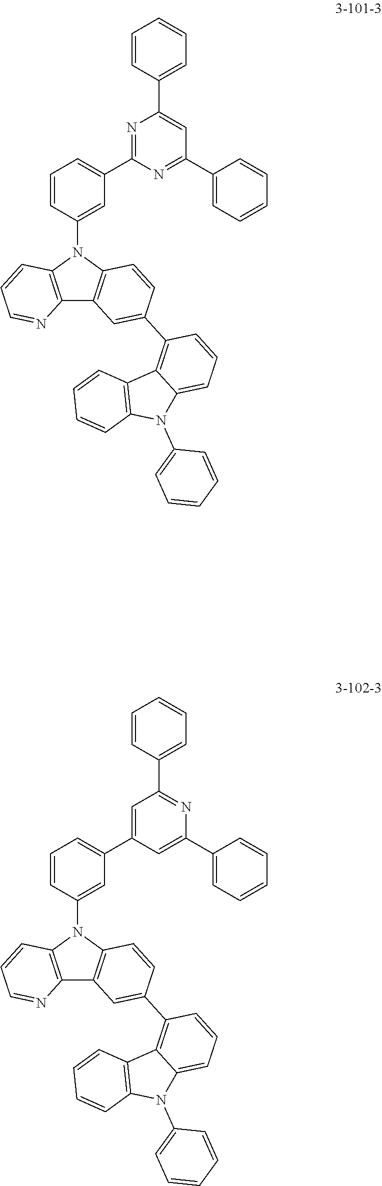 [SMILES: c1ccc(-c2cc(-c3ccccc3)nc(-c3cccc(-n4c5ccc(-c6cccc7c6c6ccccc6n7-c6ccccc6)cc5c5ncccc54)c3)n2)cc1 ; c1ccc(-c2cc(-c3cccc(-n4c5ccc(-c6cccc7c6c6ccccc6n7-c6ccccc6)cc5c5ncccc54)c3)cc(-c3ccccc3)n2)cc1]

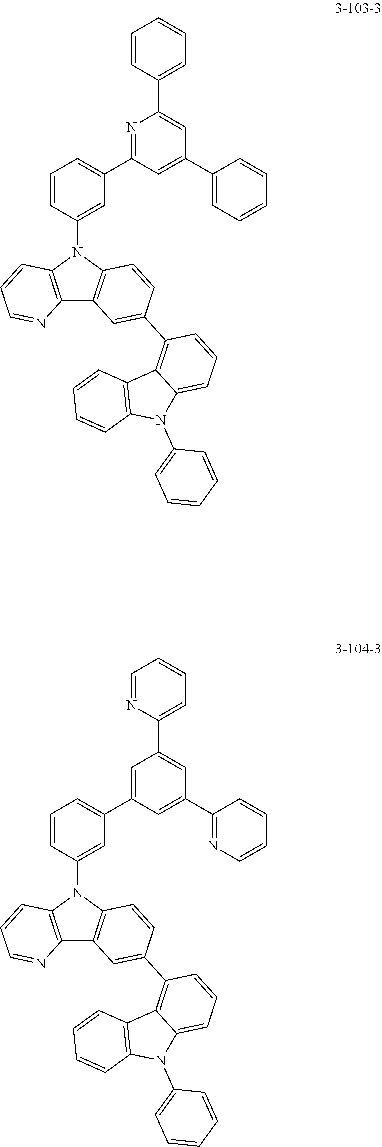 [SMILES: c1ccc(-c2cc(-c3ccccc3)nc(-c3cccc(-n4c5ccc(-c6cccc7c6c6ccccc6n7-c6ccccc6)cc5c5ncccc54)c3)c2)cc1 ; c1ccc(-n2c3ccccc3c3c(-c4ccc5c(c4)c4ncccc4n5-c4cccc(-c5cc(-c6ccccn6)cc(-c6ccccn6)c5)c4)cccc32)cc1]

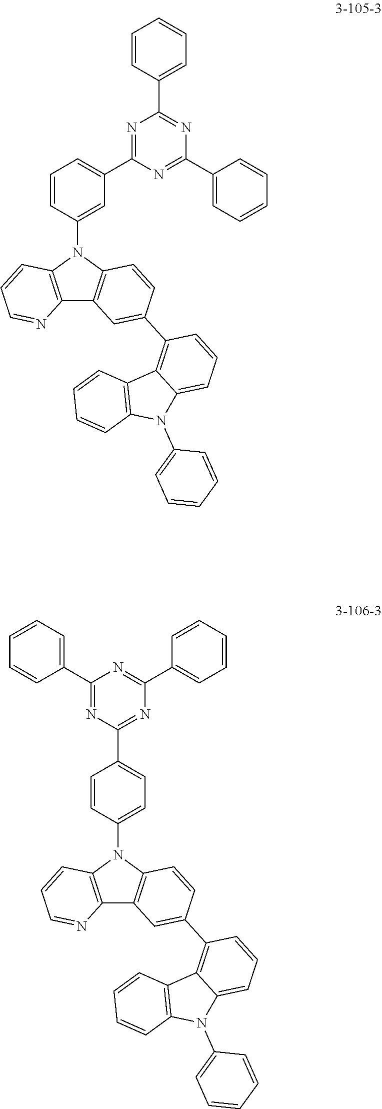 [SMILES: c1ccc(-c2nc(-c3ccccc3)nc(-c3cccc(-n4c5ccc(-c6cccc7c6c6ccccc6n7-c6ccccc6)cc5c5ncccc54)c3)n2)cc1 ; c1ccc(-c2nc(-c3ccccc3)nc(-c3ccc(-n4c5ccc(-c6cccc7c6c6ccccc6n7-c6ccccc6)cc5c5ncccc54)cc3)n2)cc1]

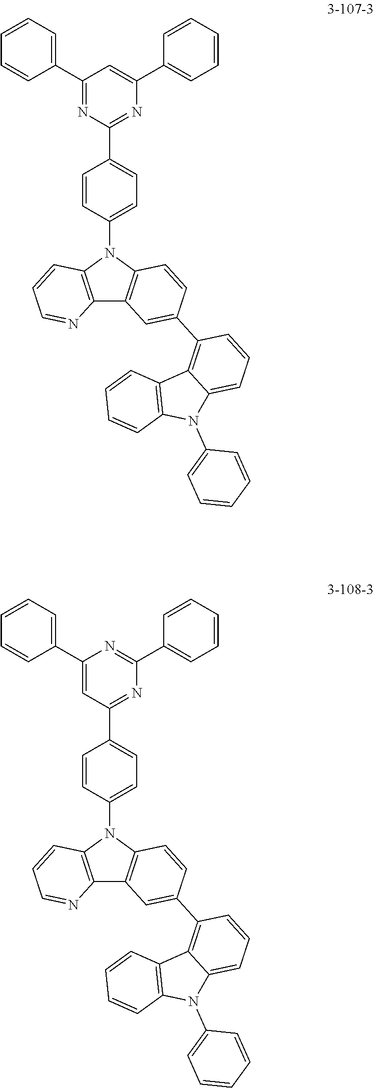 [SMILES: c1ccc(-c2cc(-c3ccccc3)nc(-c3ccc(-n4c5ccc(-c6cccc7c6c6ccccc6n7-c6ccccc6)cc5c5ncccc54)cc3)n2)cc1 ; c1ccc(-c2cc(-c3ccc(-n4c5ccc(-c6cccc7c6c6ccccc6n7-c6ccccc6)cc5c5ncccc54)cc3)nc(-c3ccccc3)n2)cc1]

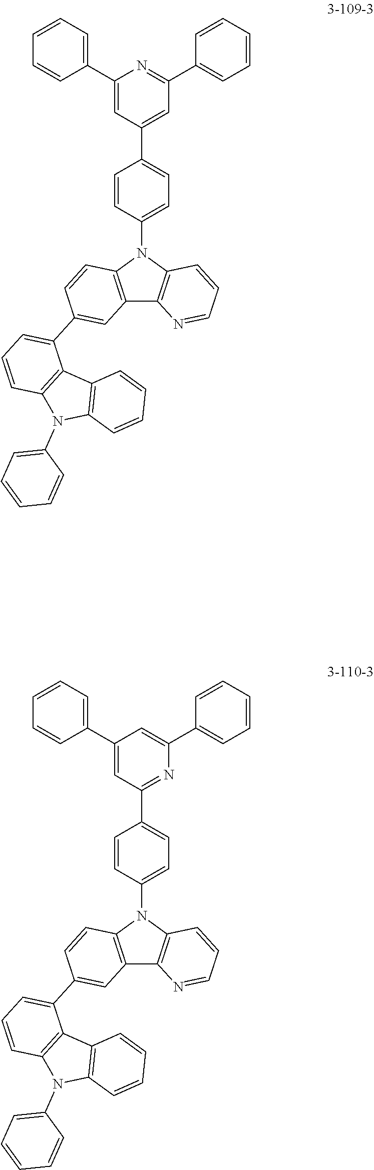 [SMILES: c1ccc(-c2cc(-c3ccc(-n4c5ccc(-c6cccc7c6c6ccccc6n7-c6ccccc6)cc5c5ncccc54)cc3)cc(-c3ccccc3)n2)cc1 ; c1ccc(-c2cc(-c3ccccc3)nc(-c3ccc(-n4c5ccc(-c6cccc7c6c6ccccc6n7-c6ccccc6)cc5c5ncccc54)cc3)c2)cc1]

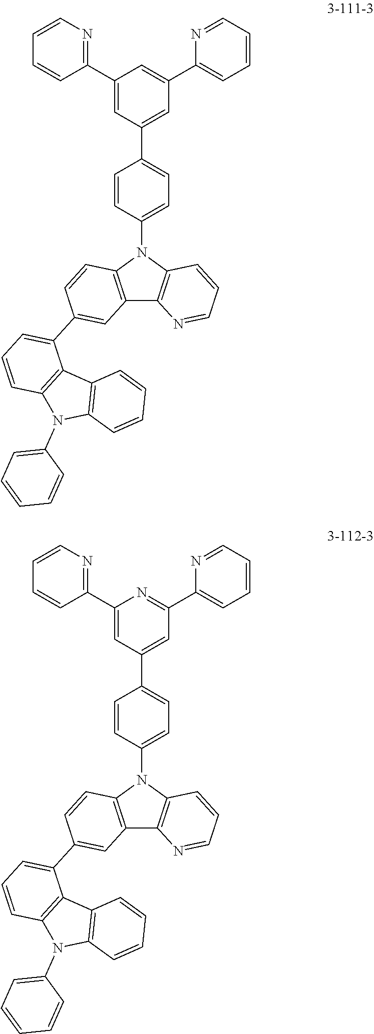

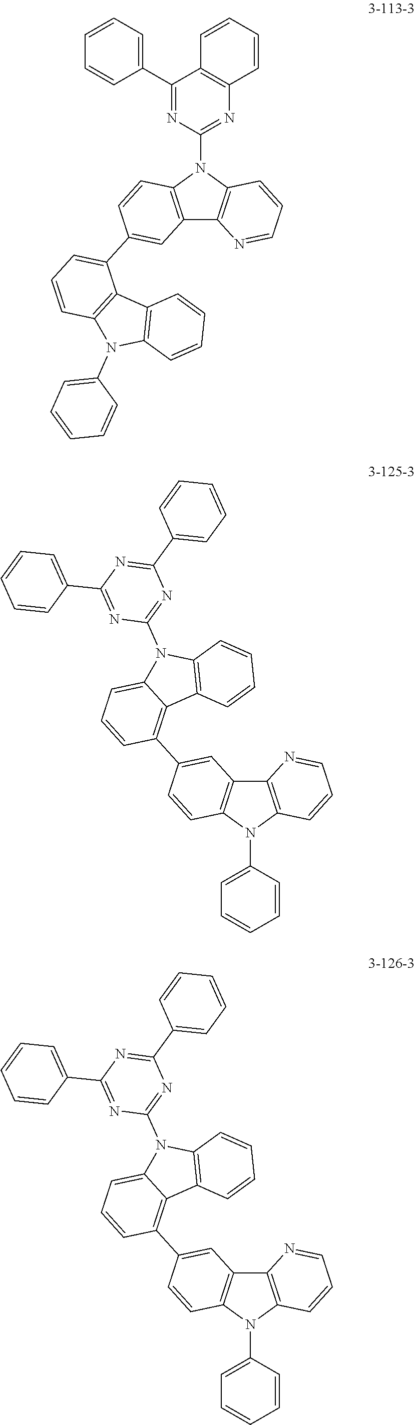 [SMILES: c1ccc(-c2nc(-n3c4ccc(-c5cccc6c5c5ccccc5n6-c5ccccc5)cc4c4ncccc43)nc3ccccc23)cc1 ; c1ccc(-c2nc(-c3ccccc3)nc(-n3c4ccccc4c4c(-c5ccc6c(c5)c5ncccc5n6-c5ccccc5)cccc43)n2)cc1 ; c1ccc(-c2nc(-c3ccccc3)nc(-n3c4ccccc4c4c(-c5ccc6c(c5)c5ncccc5n6-c5ccccc5)cccc43)n2)cc1]

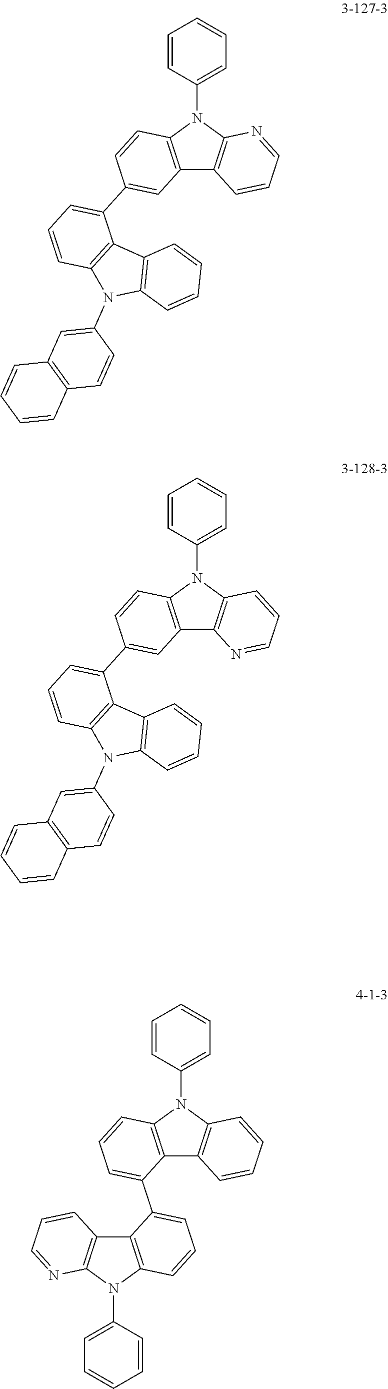 [SMILES: c1ccc(-n2c3ccc(-c4cccc5c4c4ccccc4n5-c4ccc5ccccc5c4)cc3c3cccnc32)cc1 ; c1ccc(-n2c3ccc(-c4cccc5c4c4ccccc4n5-c4ccc5ccccc5c4)cc3c3ncccc32)cc1 ; c1ccc(-n2c3ccccc3c3c(-c4cccc5c4c4cccnc4n5-c4ccccc4)cccc32)cc1]

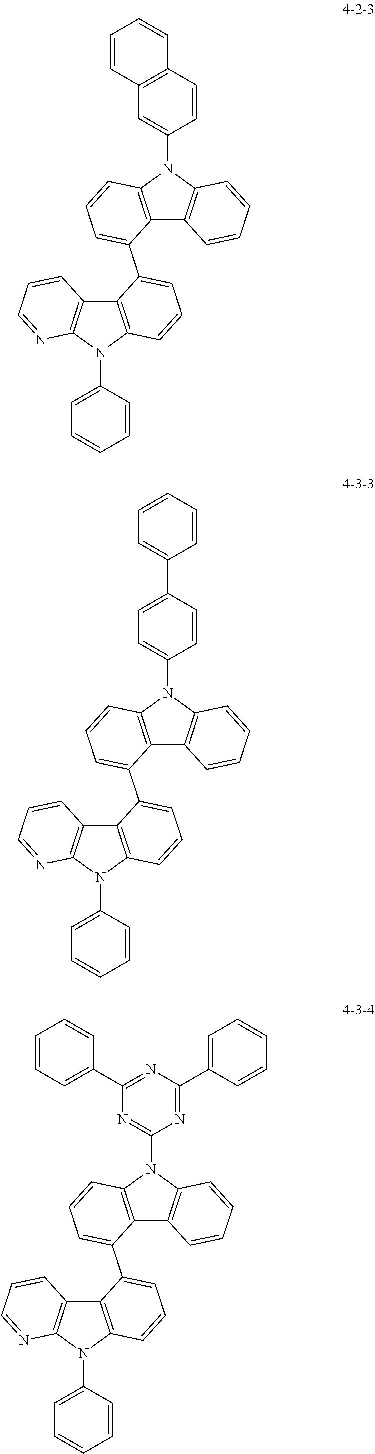 [SMILES: c1ccc(-n2c3cccc(-c4cccc5c4c4ccccc4n5-c4ccc5ccccc5c4)c3c3cccnc32)cc1 ; c1ccc(-c2ccc(-n3c4ccccc4c4c(-c5cccc6c5c5cccnc5n6-c5ccccc5)cccc43)cc2)cc1 ; c1ccc(-c2nc(-c3ccccc3)nc(-n3c4ccccc4c4c(-c5cccc6c5c5cccnc5n6-c5ccccc5)cccc43)n2)cc1]

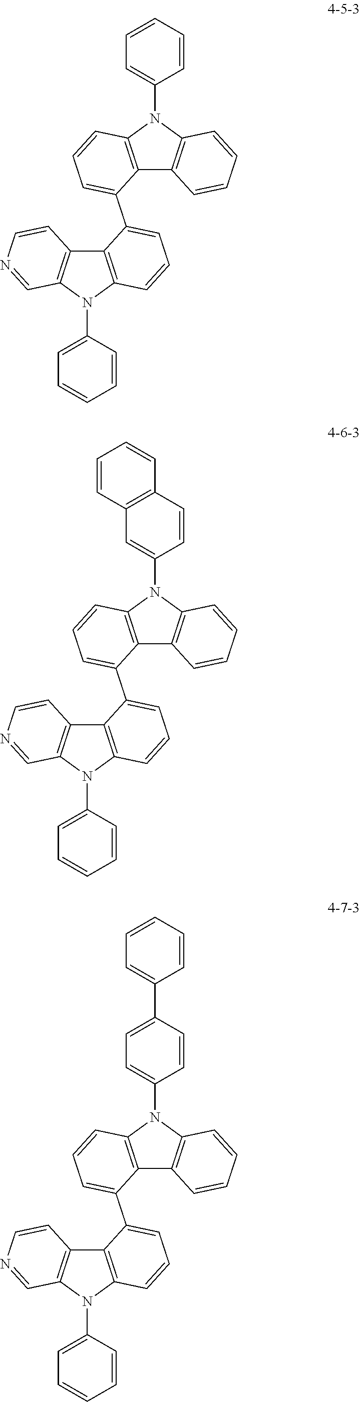 [SMILES: c1ccc(-n2c3ccccc3c3c(-c4cccc5c4c4ccncc4n5-c4ccccc4)cccc32)cc1 ; c1ccc(-n2c3cnccc3c3c(-c4cccc5c4c4ccccc4n5-c4ccc5ccccc5c4)cccc32)cc1 ; c1ccc(-c2ccc(-n3c4ccccc4c4c(-c5cccc6c5c5ccncc5n6-c5ccccc5)cccc43)cc2)cc1]

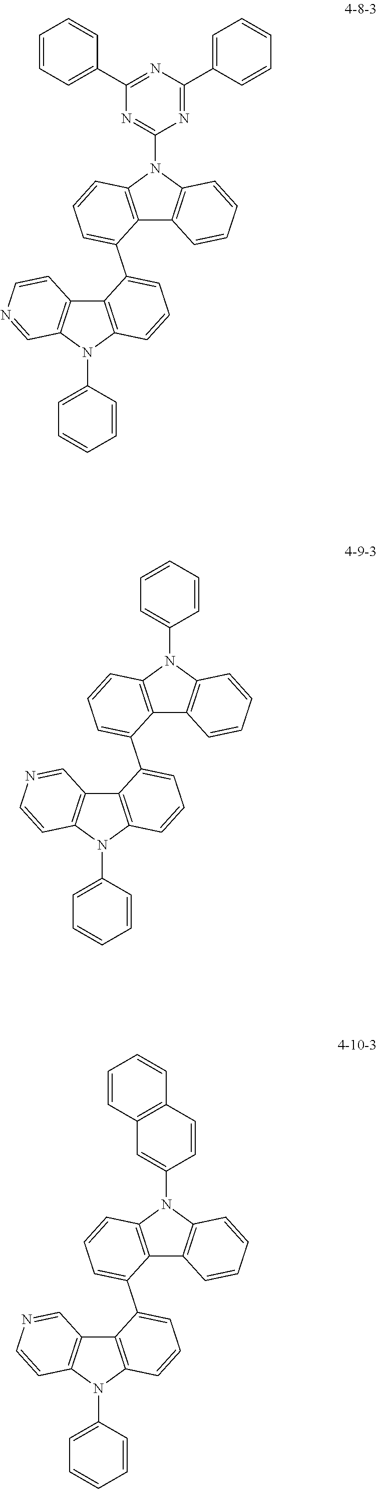 [SMILES: c1ccc(-c2nc(-c3ccccc3)nc(-n3c4ccccc4c4c(-c5cccc6c5c5ccncc5n6-c5ccccc5)cccc43)n2)cc1 ; c1ccc(-n2c3ccccc3c3c(-c4cccc5c4c4cnccc4n5-c4ccccc4)cccc32)cc1 ; c1ccc(-n2c3ccncc3c3c(-c4cccc5c4c4ccccc4n5-c4ccc5ccccc5c4)cccc32)cc1]

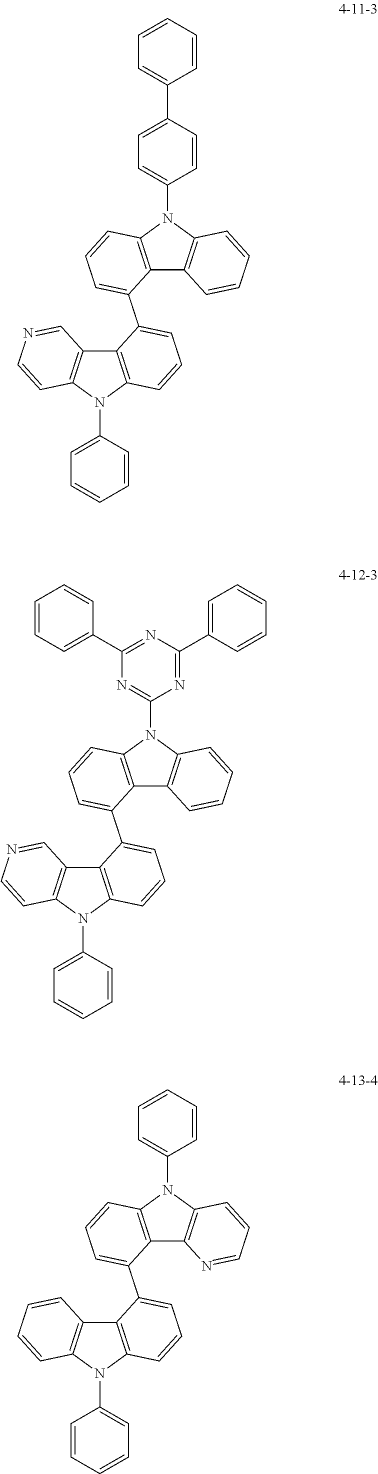 [SMILES: c1ccc(-c2ccc(-n3c4ccccc4c4c(-c5cccc6c5c5cnccc5n6-c5ccccc5)cccc43)cc2)cc1 ; c1ccc(-c2nc(-c3ccccc3)nc(-n3c4ccccc4c4c(-c5cccc6c5c5cnccc5n6-c5ccccc5)cccc43)n2)cc1 ; c1ccc(-n2c3ccccc3c3c(-c4cccc5c4c4ncccc4n5-c4ccccc4)cccc32)cc1]

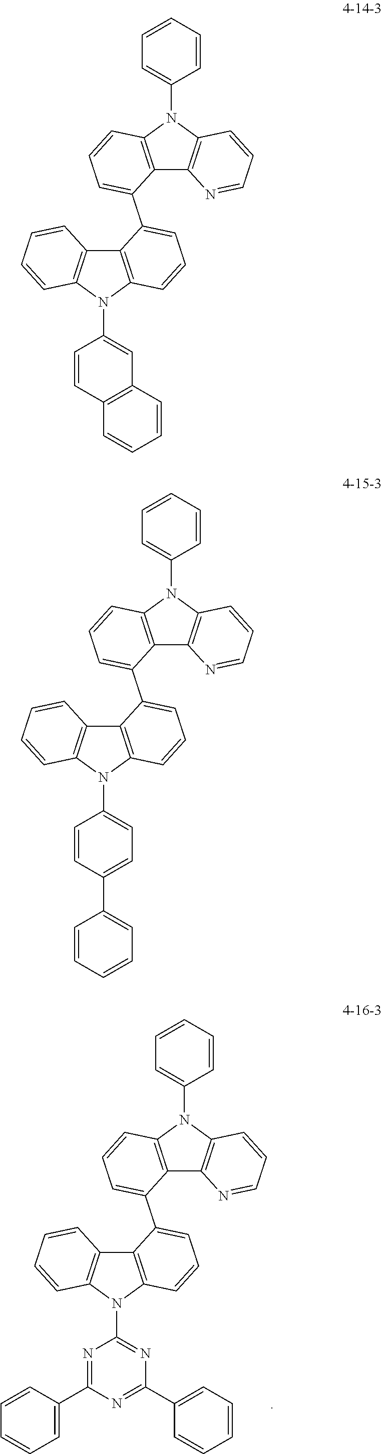 [SMILES: c1ccc(-n2c3cccnc3c3c(-c4cccc5c4c4ccccc4n5-c4ccc5ccccc5c4)cccc32)cc1 ; c1ccc(-c2ccc(-n3c4ccccc4c4c(-c5cccc6c5c5ncccc5n6-c5ccccc5)cccc43)cc2)cc1 ; c1ccc(-c2nc(-c3ccccc3)nc(-n3c4ccccc4c4c(-c5cccc6c5c5ncccc5n6-c5ccccc5)cccc43)n2)cc1]

10. An organic electronic element, comprising:
a first electrode;
a second electrode; and
an organic material layer positioned between the first electrode and the second electrode and containing the compound of claim 1.

11. The organic electronic element of claim 14, wherein the organic material layer includes a light emitting layer, the compound being contained alone or as a mixture in the light emitting layer.

12. The organic electronic element of claim 14, further comprising a light efficiency improving layer formed on at least one of one surface of the first electrode or one surface of the second electrode, which is opposite to the organic material layer.

13. The organic electronic element of claim 14, wherein the organic material layer is formed by a spin coating process, a nozzle printing process, an inkjet printing process, a slot coating process, a dip coating process, or a roll-to-roll process.

14. An electronic device, comprising:
a display device comprising the organic electronic element of claim 14; and
a controller driving the display device.

15. The electronic device of claim 18, wherein the organic electric element is one of an organic electronic light emitting element, an organic solar cell, an organic photo conductor, an organic transistor, and an element for a monochromatic or white illumination.
